SEQUENCE LISTING (6 sequences)

SEQ ID NO: 1 - protein, 155 aa, Homo sapiens [sequence omitted]

SEQ ID NO: 2 - DNA, 474 bp, Homo sapiens
gggaccatggcagccgggagcatcaccacgctgcccgccttgcccgaggatggcggcagcggcgccttcccgcccggccacttcaaggaccccaagcggctgtactgcaaaaacgggggcttcttcctgcgcatccaccccgacggccgagttgacggggtccgggagaagagcgaccctcacatcaagctacaacttcaagcagaagagagaggagttgtgtctatcaaaggagtgtgtgctaaccgttacctggctatgaaggaagatggaagattactggcttctaaatgtgttacggatgagtgtttcttttttgaacgattggaatctaataactacaatacttaccgctcaaggaaatacaccagttggtatgtggcactgaaacgaactgggcagtataaacttggatccaaaacaggacctgggcagaaagctatactttttcttccaatgtctgctaagagctga

SEQ ID NO: 3 - protein, 822 aa, Homo sapiens [sequence omitted]

SEQ ID NO: 4 - DNA, 2733 bp, Homo sapiens
  misc_feature (1)..(2733): n may be a or g or c or t/u
cgaggcggaacctccagcccgagcgagggtcagtttgaaaaggaggatcgagctcanctgtggagtatccatggagatgtggagccttgtcaccaacctctaactgcagaactgggnatgtggagctggaagtgcctcctcttctgggctgtgctggtcacagccacactctgcaccgctaggccgtccccgaccttgcctgaacaagcccagccctggggagcccctgtggaagtggagtccttcctggtccaccccggtgacctgctgcagcttcgctgtcggctgcgggacgatgtgcagagcatcaactggctgcgggacggggtgcagctggcggaaagcaaccgcacccgcatcacaggggaggaggtggaggtgcaggactccgtgcccgcagactccggcctctatgcttgcgtaaccagcagcccctcgggcagtgacaccacctacttctccgtcaatgtttcagatgctctcccctcctcggaggatgatgatgatgatgatgactcctcttcagaggagaaagaaacagataacaccaaaccaaaccgtatgcccgtagctccatattggacatccccagaaaagatggaaaagaaattgcatgcagtgccggctgccaagacagtgaagttcaaatgcccttccagtgggaccccaaaccccacactgcgctggttgaaaaatggcaaagaattcaaacctgaccacagaattggaggctacaaggtccgttatgccacctggagcatcataatggactctgtggtgccctctgacaagggcaactacacctgcattgtggagaatgagtacggcagcatcaaccacacataccagctggatgtcgtggagcggtcccctcaccggcccatcctgcaagcagggttgcccgccaacaaaacagtggccctgggtagcaacgtggagttcatgtgtaaggtgtacagtgacccgcagccgcacatccagtggctaaagcacatcgaggtgaatgggagcaagattggcccagacaacctgccttatgtccagatcttgaagactgctggagttaataccaccgacaaagagatggaggtgcttcacttaagaaatgtctcctttgaggacgcaggggagtatacgtgcttggcgggtaactctatcggactctcccatcactctgcatggttgaccgttctggaagccctggaagagaggccggcagtgatgacctcgcccctgtacctggagatcatcatctattgcacaggggccttcctcatctcctgcatggtggggtcggtcatcgtctacaagatgaagagtggtaccaagaagagtgacttccacagccagatggctgtgcacaagctggccaagagcatccctctgcgcagacaggtaacagtgtctgctgactccagtgcatccatgaactctggggttcttctggttcggccatcacggctctcctccagtgggactcccatgctagcaggggtctctgagtatgagcttcccgaagaccctcgctgggagctgcctcgggacagactggtcttaggcaaacccctgggagagggctgctttgggcaggtggtgttggcagaggctatcgggctggacaaggacaaacccaaccgtgtgaccaaagtggctgtgaagatgttgaagtcggacgcaacagagaaagacttgtcagacctgatctcagaaatggagatgatgaagatgatcgggaagcataagaatatcatcaacctgctgggggcctgcacgcaggatggtcccttgtatgtcatcgtggagtatgcctccaagggcaacctgcgggagtacctgcaggcccggaggcccccagggctggaatactgctacaaccccagccacaacccagaggagcagctctcctccaaggacctggtgtcctgcgcctaccaggtggcccgaggcatggagtatctggcctccaagaagtgcatacaccgagacctggcagccaggaatgtcctggtgacagaggacaatgtgatgaagatagcagactttggcctcgcacgggacattcaccacatcgactactataaaaagacaaccaacggccgactgcctgtgaagtggatggcacccgaggcattatttgaccggatctacacccaccagagtgatgtgtggtctttcggggtgctcctgtgggagatcttcactctgggcggctccccataccccggtgtgcctgtggaggaacttttcaagctgctgaaggagggtcaccgcatggacaagcccagtaactgcaccaacgagctgtacatgatgatgcgggactgctggcatgcagtgccctcacagagacccaccttcaagcagctggtggaagacctggaccgcatcgtggccttgacctccaaccaggagtacctggacctgtccatgcccctggaccagtactcccccagctttcccgacacccggagctctacgtgctcctcaggggaggattccgtcttctctcatgagccgctgcccgaggagccctgcctgccccgacacccagcccagcttgccaatcggggactcaaacgccgctgactgccacccacacgccctccccagactccaccgtcagctgtaaccctcacccacagcccctgctgggcccaccacctgtccgtccctgtcccctttcctgctggcagccggctgcctaccaggggccttcctgtgtggcctgcttca

SEQ ID NO: 5 - protein, 155 aa, Homo sapiens
MAEGEITTFTALTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 6 - DNA, 2357 bp, Homo sapiens
gagccgggctactctgagaagaagacaccaagtggattctgcttcccctgggacagcactgagcgagtgtggagagaggtacagccctcggcctacaagctctttagtcttgaaagcgccacaagcagcagctgctgagccatggctgaaggggaaatcaccaccttcacagccctgaccgagaagtttaatctgcctccagggaattacaagaagcccaaactcctctactgtagcaacgggggccacttcctgaggatccttccggatggcacagtggatgggacaagggacaggagcgaccagcacattcagctgcagctcagtgcggaaagcgtgggggaggtgtatataaagagtaccgagactggccagtacttggccatggacaccgacgggcttttatacggctcacagacaccaaatgaggaatgtttgttcctggaaaggctggaggagaaccattacaacacctatatatccaagaagcatgcagagaagaattggtttgttggcctcaagaagaatgggagctgcaaacgcggtcctcggactcactatggccagaaagcaatcttgtttctccccctgccagtctcttctgattaaagagatctgttctgggtgttgaccactccagagaagtttcgaggggtcctcacctggttgacccaaaaatgttcccttgaccattggctgcgctaacccccagcccacagagcctgaatttgtaagcaacttgcttctaaatgcccagttcacttctttgcagagccttttacccctgcacagtttagaacagagggaccaaattgcttctaggagtcaactggctggccagtctgggtctgggtttggatctccaattgcctcttgcaggctgagtccctccatgcaaaagtggggctaaatgaagtgtgttaaggggtcggctaagtgggacattagtaactgcacactatttccctctactgagtaaaccctatctgtgattcccccaaacatctggcatggctcccttttgtccttcctgtgccctgcaaatattagcaaagaagcttcatgccaggttaggaaggcagcattccatgaccagaaacagggacaaagaaatccccccttcagaacagaggcatttaaaatggaaaagagagattggattttggtgggtaacttagaaggatggcatctccatgtagaataaatgaagaaagggaggcccagccgcaggaaggcagaataaatccttgggagtcattaccacgccttgaccttcccaaggttactcagcagcagagagccctgggtgacttcaggtggagagcactagaagtggtttcctgataacaagcaaggatatcagagctgggaaattcatgtggatctggggactgagtgtgggagtgcagagaaagaaagggaaactggctgaggggataccataaaaagaggatgatttcagaaggagaaggaaaaagaaagtaatgccacacattgtgcttggcccctggtaagcagaggctttggggtcctagcccagtgcttctccaacactgaagtgcttgcagatcatctggggacctggtttgaatggagattctgattcagtgggttgggggcagagtttctgcagttccatcaggtcccccccaggtgcaggtgctgacaatactgctgccttacccgccatacattaaggagcagggtcctggtcctaaagagttattcaaatgaaggtggttcgacgccccgaacctcacctgacctcaactaacccttaaaaatgcacacctcatgagtctacctgagcattcaggcagcactgacaatagttatgcctgtactaaggagcatgattttaagaggctttggccaatgcctataaaatgcccatttcgaagatatacaaaaacatacttcaaaaatgttaaacccttaccaacagcttttcccaggagaccatttgtattaccattacttgtataaatacacttcctgcttaaacttgacccaggtggctagcaaattagaaacaccattcatctctaacatatgatactgatgccatgtaaaggcctttaataagtcattgaaatttactgtgagactgtatgttttaattgcatttaaaaatatatagcttgaaagcagttaaactgattagtattcaggcactgagaatgatagtaataggatacaatgtataagctactcacttatctgatacttatttacctataaaatgagatttttgttttccactgtgctattacaaattttcttttgaaagtaggaactcttaagcaatggtaattgtgaataaaaattgatgagagtgttaaaaaaaaaaaaaaaaa

(19) United States
(12) Patent Application Publication
(10) Pub. No.: US 2005/0187150 A1
(43) Pub. Date: Aug. 25, 2005

(54) Structure-based design and synthesis of FGF inhibitors and FGF modulator compounds

(75) Inventors: Moosa Mohammadi, New York, NY (US); David L. Green, Scarsdale, NY (US); Linhardt J. Robert, Iowa City, IA (US)

Correspondence Address: DARBY & DARBY P.C., P. O. BOX 5257, NEW YORK, NY, 10150-5257, US

(73) Assignees: New York University; University of Iowa Research Foundation

(21) Appl. No.: 10/285,405

(22) Filed: Oct. 31, 2002

Related U.S. Application Data
(60) Provisional application No. 60/335,583, filed on Oct. 31, 2001.

(51) Int. Cl. A61K 38/00; C07K 14/00

(52) U.S. Cl. 514/12; 530/300

(57) ABSTRACT

The present invention provides methods and compositions for modulating FGF-signaling and activities associated therewith, such as mitogenesis and angiogenesis. In particular, the invention provides crystal structure coordinates for a ternary complex of an FGF receptor, and FGF ligand, and a third compound, sucrose octasulfate, that binds to the FGF receptor and ligand to promote formation and dimerization of the ternary complex. Screening methods are provided by which novel agonists and antagonist for FGF-mediating signaling and activities may be identified using these crystal structure coordinates. Exemplary compounds are also provided that have novel utilities as agonists or antagonists of FGF-mediated signaling and activites.

### CROSS-REFERENCE TO RELATED APPLICATION(S)

Priority is claimed under 35 U.S.C. § 119(e) to U.S. provisional patent application serial No. 60/335,583 filed on Oct. 31, 2002, which is incorporated herein by reference in its entirety.

### STATEMENT REGARDING FEDERALLY SPONSORED RESEARCH AND/OR DEVELOPMENT

This invention was made with Government support under Grant Nos. 1R01-DE13686-01, 1RO1-HL52622 and 1RO1-HL62244, awarded by the National Institutes of Health. The United States Government may have certain rights to this invention pursuant to the terms of those grants.

### FIELD OF THE INVENTION

The present invention relates to a class of proteins known as fibroblast growth factor (FGF) proteins or FGF ligands. The invention also relates to receptors, known as fibroblast growth factor receptors (FGFRs), that recognize and specifically bind to FGF proteins. More specifically, the invention relates to novel uses of compounds such as sucrose octasulfate (SOS), myo-inositol hexasulfate, cyclodextrin (particularly sulfated β-cyclodextrin) and suramin to modulate biological activity associated with FGF. The invention also relates to uses of such compounds to modulate dimerization of FGF-FGFR complexes.

### BACKGROUND OF THE INVENTION

The mammalian fibroblast growth factor (FGF) family comprises at least 22 related polypeptides that are generally known in the art as FGF1-FGF22. These polypeptides are known to be essential for normal human development and, moreover, are involved in the pathologies of many human diseases such as cancer and dwarfism, to name a few. For reviews, see McKeehan et al., Progress in Nucleic Acid Research and Molecular Biology 1998, 59:135-176; Nishimura et al., Biochim. Biophys. Acta. 2000, 1492:203-206; and Yamashita et al., Biochem. Biophys. Res. Commun. 2000, 277:494-498.

The diverse effects of FGF polypeptides are mediated by at least four receptor tyrosine kinase polypeptides, referred to collectively as the FGF receptors (FGFRs), and known individually as FGFR1-FGFR4. These FGFR polypeptides comprise an extracellular domain, a single transmembrane helix domain, and a cytoplasmic portion with tyrosine kinase activity. The FGFR polypeptides' extracellular domain itself has at least three immunoglobulin (Ig)-like domains, which are referred to respectively as D1-D3. The receptors' binding specificity resides in, and is therefore incurred by, the D2 and D3 and by the short linker polypeptide sequence between those two domains. See, Plotnikov et al., Cell 1999, 98:641-650; Plotnikov et al., Cell 2000, 101:413-424; and Stauber et al., Proc. Natl. Acad. Sci. U.S.A. 2000, 97:49-54 for a more detailed discussion.

FGF-induced FGFR dimerization is a key event in FGF signaling processes (Schlessinger, 2000). However, whereas other known growth factors such as platelet-derived growth factor (PDGF), neurotrophic growth factor (NGF) and colony stimulating growth factor 1 (CSF1) are themselves dimeric molecules, the FGF polypeptides are monomeric molecules and do not form dimers by themselves in solution. Consequently, FGF polypeptides cannot induce receptor dimerization by themselves and instead require soluble or cell surface-bound heparan sulfate proteoglycans (HSPG) to promote FGFR dimerization and subsequent activation.

The crystal structure determined for one FGF-FGFR-heparin complex (see, Schlessinger et al., Molecular Cell 2000, 6:743-750) indicates one putative mechanism by which heparin may facilitate FGFR dimerization. Without being limited to any particular theory or mechanism of interaction, such dimerization is believed to occur according to a “two end” model in which the non-reducing end of heparin interacts with heparin binding sites of the FGF and FGFR polypeptides to promote formation of a ternary FGF:FGFR:heparin complex of 1:1:1 stoichiometry. A second ternary FGF:FGFR:heparin complex is then recruited to this first complex by means of interactions of (i) FGFR, FGF and heparin in the first complex, with (ii) FGFR in the second complex.

The central role played by heparin for the dimerization, and hence activation, of FGF receptor polypeptides makes heparin's interactions with FGF and FGFR attractive targets for compounds which may modulate FGF receptor activity. Compounds that modulate this interactions would be useful as therapeutic agents, e.g., for the treatment of disorders associated with FGFR activity. However, the capabilities that are currently available for large-scale preparation of homogenous heparin oligosaccharides suitable for therapeutic applications are severely limited (see, Pervin et al., Glycobiology 1995, 5:83-95). There exists, therefore, a need for identifying other molecules which modulate the dimerization of FGF receptor polypeptides (e.g., by interfering with the stabilizing interactions of heparin), and which may therefore be useful, e.g., as therapeutic agents to modulate FGF receptor activity and to treat disorders associated with such activity.

It has also been suggested that some other sulfated compounds may also bind to an FGF ligand in place of heparin. For example, sucrose octasulfate (SOS) is marketed as an aluminum salt in CARAFATE® or sucralfate, a pharmaceutical composition used to treat duodenal ulcers (see, the Physician's Desk Reference, 54 Ed., 2000, Medical Economics Company, Inc., Montvale, N.J.). The mechanisms by which the compound heals ulcers are largely unknown. However, it has been suggested that SOS may promote healing by binding to and stabilizing FGFs against denaturation in the acidic pH of the stomach (Folkman et al., Ann. Surg. 1991, 214:414-425; see, also, Volkin et al., Biochimica et Biophysica Acta 1993, 1203:18-26). A crystal structure of SOS bound to FGF1 also shows that SOS stabilizes FGF by neutralizing the positively charged high affinity heparin binding residues in FGF (Zhu et al., Structure 1993, 1:27-34). The FGF ligand is also known to bind inositol hexasulfate (Pineda-Lucena, J. Mol. Biol. 1994, 42:81-98) and to suramin (Middaugh et al., Biochemistry 1992, 31:9016-9024). However, whereas inositol hexasulfate may function as a substitute for heparin to activate FGF signaling (Pineda-Lucena et al., supra), suramin actually inhibits signaling by FGF (Middaugh et al., supra).

Despite these teachings, it is not currently known in the art whether these compounds may also mediate or inhibit dimerization of FGF receptor molecules. Indeed, the exact mechanism(s) by which such compounds activate or inhibit FGF signaling remain unknown. The knowledge of such particular interactions may greatly facilitate the identification and/or screening of novel compounds that may be used as therapeutic agents (e.g., to modulate FGF signaling and/or activities associated therewith). However, in the absence of such knowledge, candidate compounds may only be identified by a completely haphazard and random screening of different guidance, with no ability to determine what compounds may or may not be reasonably expected to work.

### SUMMARY OF THE INVENTION

The present invention seeks to overcome problems in the prior art by providing ternary complexes of: (a) an FGF ligand; (b) an FGF receptor; and (c) a heparin agonist or antagonist, that is to a say a compound that mimics the binding of heparin and heparan sulfate to the FGF ligand and receptor. Crystalline forms of such ternary complexes are also described, and crystal structure coordinates for these forms are provided.

In particular, Applicants have discovered that small, preferably sulfated molecules such as sucrose octasulfate (SOS) and its derivatives, are able to specifically and simultaneously bind to FGF ligands and FGFR polypeptides and augment binding of an FGF ligand to its receptor. Moreover, such compounds are also able to stabilize dimers of the resulting ternary complexes, effectively promoting dimerization of the FGF-FGFR complexes. Using such ternary complexes and crystal structure coordinates thereof, it is possible to identify compounds that may modulate FGF-mediated signaling and/or activities associated with such signaling. For example, the ternary complexes of this invention may be used to identify compounds that form a dimerization incompetent ternary complex with an FGF ligand and FGF receptor. Such compounds are then expected to be useful, e.g., for inhibiting FGF-mediating signaling or an activity associated therewith. For example, compounds identified by these screening methods may be used to modulate tyrosine kinase activity of an FGF receptor, or they may modulate an activity such as mitogenesis, angiogenesis, cell growth (including tumor cell growth or tumor growth) that are associated with FGF signaling. The compounds are useful, e.g., in therapeutic methods and formulations, to treat or ameliorate disorders that are associated with FGF-signaling, including cell proliferative disorders such as cancer.

The invention also provides compounds that have novel uses as modulators of FGF-signaling or an activity mediated thereby. In preferred embodiments, the compounds are derivatives of sucrose octasulfate.

Thus, in preferred embodiments, compounds used in the methods and compositions of the invention may have the structure: 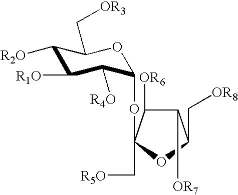
in which R₁, R₂, R₃, R₄, R₅, R₆, R₇ and R₈ are independently benzyl, trityl or —SO₃H. Preferably at least one of R₁, R₂, R₃, R₄, R₅, R₆, R₇ and R₈ is either benzyl or trityl. Particularly preferred, exemplary compounds are described in the Examples, infra, and their structures are set forth in FIG. 8 (Structures I and II), in FIG. 9 (Structure III), in FIG. 10 (Structure IV) and in FIG. 11 (Structures V and VI).

In other preferred embodiments, compounds that may be used in the methods and compositions of this invention include cyclodextrin compounds, particularly sulfated cyclodextrin compounds and sulfonated cyclodextrin compounds. The cyclodextrin compounds used may be, e.g., an α-cyclodextrin compound, a β-cyclodextrin compound or a γ-cyclodextrin compound, with β-cyclodextrin compounds being particularly preferred.

Still other compounds may also be used in the methods and compositions of this invention, including but not limited to inositol hexasulfate and suramin and their derivatives may also be used.

### BRIEF DESCRIPTION OF THE DRAWINGS

FIGS. 1A-1B present the amino acid sequence (FIG. 1A) of an exemplary FGF polypeptide, known as FGF2 (SEQ ID NO:1), along with an exemplary FGF2 nucleic acid sequence (FIG. 1B; SEQ ID NO:2) having an open reading frame ( ) that encodes this FGF2 polypeptide. The FGF2 polypeptide sequence (SEQ ID NO:1) is available from GenBank and has the Accession No. P09038 (GI:122742). The nucleic acid sequence (SEQ ID NO:2) is also available from GenBank and has the Accession No. M17599.1 (GI:183086).

FIGS. 2A-2B present the amino acid sequence (FIG. 2A) for an exemplary FGF receptor polypeptide, known as FGFR1 (SEQ ID NO:3), along with an exemplary FGFR1 nucleic acid sequence (FIG. 2B; SEQ ID NO:4) having an open reading frame that encodes this FGFR1 polypeptide. The FGFR1 polypeptide sequence (SEQ ID NO:3) is available from GenBank and has the Accession Number P11362 (GI:120046). The nucleic acid sequence is also available from GenBank and has the Accession No. X51803.1 (GI:31367).

FIGS. 3A-D show chromatograms obtained from aliquots of purified 1:1 molar ratios of FGF2:FGFR1 complexes (2 mg) mixed with various molar ratios of sucrose octasulfate (SOS) and analyzed on a Superdex 200 size exclusion column in 25 mM HEPES-NaOH buffer (pH 7.5) containing 150 mM NaCl. The elution positions of monomers and dimers of the FGF2:FGFR1 complexes are indicated by the letters M and D, respectively. The letter L indicates the position of free FGF2 resulting from dissociation of FGF2:FGFR1 complexes due to protein dilution during the size exclusion chromatography. FIG. 3A shows the size exclusion chromatogram for a control solution that contains no SOS. FIG. 3B shows the size exclusion chromatogram when SOS was added at a molar ratio of 1:1:0.25 FGF2:FGFR1:SOS. FIG. 3C shows the size exclusion chromatogram when SOS was added at a molar ratio of 1:1:0.5 FGF2:FGFR1:SOS. FIG. 3D shows the size exclusion chromatogram when SOS was added at a molar ratio of 1:1:1 FGF2:FGFR1:SOS.

FIG. 4 graphically presents average daily counts and standard deviations of viable BaF3 cells that were transfected to stably express FGFR1 and cultured in the presence of FGF2 (50 ng/ml), either alone (●), with 3 μM heparin (x) or with SOS at a concentration of 0.1 μM (◯), 0.5 μM (□), 1 μM (Δ), 5 μM (⋄) or 10 μM (+).

FIGS. 5A-C illustrated the crystal structure determined for the FGF2-FGFR1-SOS complex. FIG. 5A illustrates an exemplary orthorhombic space group P2₁2₁2₁ crystal of the FGF2-FGFR1-SOS complex. FIGS. 5B-C illustrate the overall structure of one of the two 2:2:2 FGF2-FGFR2-SOS dimers in the crystal's asymmetric unit. The structure illustrated in FIG. 5C is identical to the structure shown in FIG. 5B, as viewed when rotated 90° around the horizontal axis.

FIG. 6 is a stereo view of the Fₒ−F_{c} electron density map computed after simulated annealing with SOS omitted from the atomic model. The electron density map is computed at 2.6 Å resolution and contoured at 2.6 σ.

FIG. 7 schematically illustrates interactions between SOS, FGF2 and FGFR1 in a dimerized ternary complex of FGF2, FGFR1 and SOS. Hydrogen bonding interactions are indicated by dashed lines. Shading around the different amino acid residues indicates to which polypeptide the residue belongs: FGF2, the primary FGFR1 (i.e., the FGFR1 molecule to which FGF2 is bound) and the secondary FGFR1 molecule 110 in the dimer.

FIG. 8 illustrates the exemplary synthesis of two preferred SOS derivatives: 2-O-Bn sucrose heptasulfate (structure I) and 1′-O-Bn sucrose heptasulfate (structure II).

FIG. 9 illustrates the exemplary synthesis of another preferred SOS derivative: 1′, 2-di-O-Bn sucrose hexasulfate (structure III).

FIG. 10 illustrates the exemplary synthesis of a third preferred sulfonated sucrose derivative: 4,6-O-isopropylidene sucrose hexasulfate (Structure IV).

FIG. 11 illustrates the exemplary synthesis of two additional preferred sulfonated sucrose derivatives: 2-O-dodecanoyl sucrose hexasulfate (Structure V) and 6′-O-hexadecanoyl sucrose hexasulfate (Structure VI).

FIG. 12 illustrates the chemical structure of suramin (Structure VII).

FIG. 13 shows chromatograms obtained from aliquots of purified 1:11 molar ratios of FGF2:FGFR1 complexes (2 mg) mixed with various molar ratios of suramin and analyzed on a Superdex 200 size exclusion column in 25 mM HEPES-NaOH buffer (pH 7.5) containing 150 mM NaCl. The elution positions of monomers and dimers of the FGF2:FGFR1 complexes are indicated by the letters M and D, respectively. The letter L indicates the position of free FGF2 resulting from dissociation of FGF2:FGFR1 complexes due to protein dilution during the size exclusion chromatography. FIG. 13A shows the size exclusion chromatogram for a control solution that contains no suramin. FIG. 13B shows the size exclusion chromatogram when suramin was added at a molar ratio of 1:1:0.25 FGF2:FGFR1:suramin. FIG. 13C shows the size exclusion chromatogram when suramin is added at a molar ratio of 1:1:0.5 FGF2:FGFR1 suramin. FIG. 13D shows the size exclusion chromatogram when suramin is added at a molar ratio of 1:1:1 FGF2:FGFR1:suramin.

FIG. 14 illustrates an exemplary, general structure for derivatives of a preferred class of cyclodextrin molecule, β-cyclodextrin (Structure VIII). For sulfonated cyclodextrin molecules, each R group is independently selected and is preferably either a hydrogen group (H) or a sulfonate group (SO₃) with at least one R being a sulfonated group. For sulfated cyclodextrin molecules, each R group is independently selected and is preferably either a hydrogen group (H) or a sulfate group (SH) with at least one R being a sulfate group.

FIG. 15A graphically presents average daily counts and standard deviations of viable BaF3 cells that were transfected to stably express FGFR1 and cultured in the presence of FGF1 (50 ng/ml) either alone ( [symbol] ), with 10 μg/ml heparin (x), or with sulfonated β-cyclodextrin at concentrations of 1 μM (▴), 5 μM (♦), 10 μM (●), or 25 μM (▪).

FIGS. 15B and 15C show immunoblots of cellular proteins from BaF3 cells that overexpress FGFR1 and were incubated with FGF1 (50 ng/ml), heparin (10 μg/ml) and sulfonated β-cyclodextrin (5 and 25 μM). FIG. 15B shows protein bands that were immunoprecipitated with an anti-FGFR1 monoclonal antibody and detected using labeled antibody to phosphotyrosine. FIG. 15C shows protein bands that were immunoprecipitated with monoclonal antibodies to ERK-1 and/or ERK-2, and detected with labeled antibody to phosphotyrosine.

FIGS. 16A-16B present the amino acid sequence (FIG. 16A) of a second exemplary FGF polypeptide, known as FGF1 (SEQ ID NO:5), along with an exemplary FGF1 nucleic acid sequence (FIG. 16B; SEQ ID NO:6) having an open reading frame (nucleotides 142-609) that encodes this FGF1 polypeptide. The FGF1 polypeptide sequence (SEQ ID NO:5) is available from GenBank and has the Accession No. NP_{—}000791 (GI:4503697). The nucleic acid sequence (SEQ ID NO:6) is also available from GenBank and has the Accession No. NM_{—}000800 (GI:15055546).

### DETAILED DESCRIPTION OF THE INVENTION

The present invention relates to a particular family or class of polypeptides, referred to herein as fibroblast growth factor (FGF) polypeptides or as FGF ligands. The FGF ligands of the invention bind to a particular family or class of receptor polypeptides, that are referred to herein as FGF receptors (FGFR). Briefly, and without being limited to any particular theory or mechanism of action, the FGF ligands are believed to mediate cell signaling by specifically binding to FGFR polypeptides. Upon binding to an FGF ligand, the FGFR polypeptide then binds to a second FGFR molecule and, more preferably, binds to a second FGFR molecule that has also bound to an FGF ligand, to form a dimer complex, and a tyrosine kinase activity of the receptor is then activated. In particular, upon forming the dimer complex biological activities (such as mitogenesis, angiogenesis and/or tumor growth) that are associated with FGF signaling may be activated and/or increased.

Under normal physiological conditions, heparan sulfate proteoglycans (HSPG) are also required to promote ligand binding and/or dimerization by FGFR. In particular, and again without being limited to any particular theory or mechanism of action, heparin and HSPGs are believed to bind to the FGF ligand and its receptor, and thereby stabilize the FGF ligand-receptor complex. Moreover, the HSPG (e.g., heparin) is also believed to interact with a second FGFR molecule, thereby promoting FGFR dimerization. More specifically, it is understood that, under normal physiological conditions FGF ligand, FGFR and heparin bind to each other to form a 1:1:1 ternary complex; i.e., a complex consisting essentially of one FGF ligand molecule, one FGFR molecule, and one heparin molecule (referred to herein as the “ternary complex” or as the FGF:FGFR:heparin complex). This ternary complex is understood to form stable dimers, by binding to a second ternary complex, under normal physiological conditions, thereby activating the FGF receptor(s).

Applicants have discovered, as demonstrated in the Examples infra, that small, sulfated molecules may also form ternary complexes with an FGF receptor and its ligand. In particular, the Examples, infra, describe experiments in which sucrose octasulfate (SOS) forms a 1:1:1 ternary complex with an FGF ligand and receptor. Thus, these experiments demonstrate that small molecules such as SOS are able to act in place of heparin to stabilize binding of an FGF ligand to its receptor. Moreover, the experiments further demonstrate that SOS also stabilizes dimerization of the FGF receptor.

The Examples, infra, describe additional experiments demonstrating that other small molecules, particularly suramin, are also capable of forming 1:1:1 ternary complexes with an FGF ligand and receptor and, moreover, show that these molecules may function as antagonist of FGF-mediating signaling. Specifically, the experiments show that compounds such as suramin actually induce the formation of FGF-FGFR dimers that are signaling incompetent.

The Experiments described in the Examples, infra, additionally provide a three-dimensional structure, determined by X-ray crystallography, for a dimeric 2:2:2 FGF2:FGFR1:SOS complex (coordinates for this structure are provided in the Appendix, infra). This structure reveals particular interactions between sulfate groups of the SOS and amino acid residues of FGF and FGFR. These interactions are involved in the stabilization of (1) complexes between the FGF ligand and its receptor (more specifically, the stabilization of a 1:1:1 FGF:FGFR:SOS ternary complex); and (2) FGFR dimers (more specifically, stabilization of the ternary complex dimers).

For example, hydrogen-bonding interactions are described in Example 4, infra, between sulfate groups of the SOS molecule, and amino acid residues lysine 163 and lysine 177 of FGFR1. Hydrogen bonding interactions are also described between sulfate groups of SOS, and amino acid residues lysine 26 and lysine 135 of FGF2. Without being limited to any particular theory or mechanism of action, these hydrogen bonding interactions are believed to be involved in the stabilization of the FGF2:FGFR1:SOS ternary complex. Other hydrogen-bonding interactions are also described between sulfate groups of the SOS molecule, and amino acid residues lysine 207, glycine 205 and aspartic acid 218 of the second FGFR1 molecule in the dimer. Thus, these other hydrogen bonding interactions are expected to be involved in stabilization of dimers of the ternary complex.

Accordingly, the present invention relates to and provides a three dimensional (i.e. “tertiary”) structure for a ternary complex (preferably a dimerized ternary complex) of (i) an FGF ligand, (ii) an FGF receptor, and (iii) a small, preferably sulfated molecule that promotes formation and/or dimerization of such a ternary complex. For example, coordinates for an exemplary structure, which is a ternary complex of FGF2:FGFR1:SOS, are provided in the Appendix, infra. In preferred embodiments, the small molecule is SOS or a derivative thereof. However, the skilled artisan will appreciate that other small molecules, particularly sulfated molecules, may be used, such as inositol hexasulfate, sulfated β-cyclodextrin and suramin. The invention also relates to and provides crystals comprising an above-described ternary complex which are of suitable quality and therefore useful for determining the three dimensional structure of such a complex.

The crystals and structure of the present invention are useful, e.g., for identifying other compounds that may bind to an FGF ligand and/or its receptor and therefore modulate their activity. For example, using computer modeling algorithms and other techniques well known in the art, a user may readily use the structure provided here to identify other compounds that are expected to similarly bind to an FGF ligand and/or its receptor. Another aspect of the invention therefore involves the use of the above-mentioned structures and/or crystals to identify other compounds that interact with an FGF ligand and/or its receptor, and which may be useful, e.g., as antagonist or agonist of FGF-mediated signaling.

A skilled user may identify compounds that form or may be expected to form stabilizing interactions in a ternary complex with an FGF ligand and its receptor. In one preferred aspect, such compounds may be ones that do not form (or are not expected to form) stabilizing interactions with another ternary complex or, more specifically, with another FGF receptor. Such compounds would then be expected to inhibit dimerization of an FGF receptor, and may be used, e.g., as antagonist of an FGF receptor and/or to inhibit FGF mediated signaling and effects thereof. In another preferred aspect of such methods, the compounds identified may be ones that form (or are expected to form) improved interactions with either an FGF ligand or an FGF receptor in a ternary complex, or with a second FGF receptor (i.e., in a dimer). Such improved interactions might be, for example, hydrogen bonding or other interactions that may be either stronger or more specific that those observed for another compound (for example, stronger or more specific than interactions observed for heparin or for SOS). Compounds identified in this aspect of the invention may be expected to bind more strongly and/or more specifically with and FGF ligand and its receptor, and may also be expected to bind more strongly and/or specifically with a second FGFR molecule to form dimers. Thus, the compounds identified in this second aspect may be useful, e.g., as agonists to increase activation of an FGF receptor and/or an activity associated therewith.

Classes of compounds that may be identified by such screening assays include, but are not limited to, small molecules (e.g., organic or inorganic molecules which are less than about 2 kDa in molecular weight, are more preferably less than about 1 kDa in molecular weight, and/or are able to cross the blood-brain barrier and affect FGF-signaling or activities associated therewith) as well as macromolecules (e.g., molecules greater than about 2 kDa in molecular weight). Compounds identified by these screening assays may also include peptides and polypeptides. Examples of such compounds (including peptides) include but are not limited to: soluble peptides; fusion peptide members of combinatorial libraries (such as ones described by Lam et al., Nature 1991, 354:82-84; and by Houghten et al., Nature 1991, 354:84-86); members of libraries derived by combinatorial chemistry, such as molecular libraries of D- and/or L-configuration amino acids; phosphopeptides, such as members of random or partially degenerate, directed phosphopeptide libraries (see, e.g., Songyang et al., Cell 1993, 72:767-778); antibodies, including but not limited to polyclonal, monoclonal, humanized, anti-idiotypic, chimeric or single chain antibodies; antibody fragments, including but not limited to Fab, F(ab′)₂, Fab expression library fragments, and epitope-binding fragments thereof.

In preferred embodiments, the compounds identified in such methods are sulfated saccharides, preferably disaccharides such as sucrose octasfulate (SOS), and their derivatives. However, other small, sulfated compounds such as sulfated inositols, sulfated cyclodextrins and their derivatives may also be used. Particular exemplary compounds may include myo-inositol hexasulfate, sulfated β-cyclodextrin, and their derivatives, and suramin. Indeed, a skilled artisan will appreciate that any compound that may be modified with an FGF ligand-receptor complex (e.g., using routine computer modeling algorithms) may be used in the screening methods described here. The methods, therefore, are not limited to the particular compounds that are described in this application only to illustrate the invention.

### Definitions

The terms used in this specification generally have their ordinary meanings in the art, within the context of this invention and in the specific context where each term is used. Certain terms are discussed below, or elsewhere in the specification, to provide additional guidance to the practitioner in describing the compositions and methods of the invention and how to make and use them.

General Definitions. As used herein, the term “isolated” means that the referenced material is removed from the environment in which it is normally found. Thus, an isolated biological material can be free of cellular components, i.e., components of the cells in which the material is found or produced. In the case of nucleic acid molecules, an isolated nucleic acid includes a PCR product, an isolated mRNA, a cDNA, or a restriction fragment. In another embodiment, an isolated nucleic acid is preferably excised from the chromosome in which it may be found, and more preferably is no longer joined to non-regulatory, non-coding regions, or to other genes, located upstream or downstream of the gene contained by the isolated nucleic acid molecule when found in the chromosome. In yet another embodiment, the isolated nucleic acid lacks one or more introns. Isolated nucleic acid molecules include sequences inserted into plasmids, cosmids, artificial chromosomes, and the like. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated organelle, cell, or tissue is removed from the anatomical site in which it is found in an organism. An isolated material may be, but need not be, purified.

The term “purified” as used herein refers to material that has been isolated under conditions that reduce or eliminate the presence of unrelated materials, i.e., contaminants, including native materials from which the material is obtained. For example, a purified protein is preferably substantially free of other proteins or nucleic acids with which it is associated in a cell; a purified nucleic acid molecule is preferably substantially free of proteins or other unrelated nucleic acid molecules with which it can be found within a cell. As used herein, the term “substantially free” is used operationally, in the context of analytical testing of the material. Preferably, purified material substantially free of contaminants is at least 50% pure; more preferably, at least 90% pure, and more preferably still at least 99% pure. Purity can be evaluated by chromatography, gel electrophoresis, immunoassay, composition analysis, biological assay, and other methods known in the art.

Methods for purification are well-known in the art. For example, nucleic acids can be purified by precipitation, chromatography (including preparative solid phase chromatography, oligonucleotide hybridization, and triple helix chromatography), ultracentrifugation, and other means. Polypeptides and proteins can be purified by various methods including, without limitation, preparative disc-gel electrophoresis, isoelectric focusing, HPLC, reversed-phase HPLC, gel filtration, ion exchange and partition chromatography, precipitation and salting-out chromatography, extraction, and countercurrent distribution. For some purposes, it is preferable to produce the polypeptide in a recombinant system in which the protein contains an additional sequence tag that facilitates purification, such as, but not limited to, a polyhistidine sequence, or a sequence that specifically binds to an antibody, such as FLAG and GST. The polypeptide can then be purified from a crude lysate of the host cell by chromatography on an appropriate solid-phase matrix. Alternatively, antibodies produced against the protein or against peptides derived therefrom can be used as purification reagents. Cells can be purified by various techniques, including centrifugation, matrix separation (e.g., nylon wool separation), panning and other immunoselection techniques, depletion (e.g., complement depletion of contaminating cells), and cell sorting (e.g., fluorescence activated cell sorting [FACS]). Other purification methods are possible. A purified material may contain less than about 50%, preferably less than about 75%, and most preferably less than about 90%, of the cellular components with which it was originally associated. The “substantially pure” indicates the highest degree of purity which can be achieved using conventional purification techniques known in the art.

A “sample” as used herein refers to a biological material which can be tested, e.g., for the presence of an FGF polypeptide or FGF nucleic acid or, alternatively, for the presence of an FGFR polypeptide or nucleic acid (e.g., to identify cells that specifically express either FGF or FGFR). Such samples can be obtained from any source, including tissue, blood and blood cells, including circulating hematopoietic stem cells (for possible detection of protein or nucleic acids), plural effusions, cerebrospinal fluid (CSF), ascites fluid, and cell culture. In preferred embodiments samples are obtained from bone marrow.

Non-human animals include, without limitation, laboratory animals such as mice, rats, rabbits, hamsters, guinea pigs, etc.; domestic animals such as dogs and cats; and, farm animals such as sheep, goats, pigs, horses, and cows.

In preferred embodiments, the terms “about” and “approximately” shall generally mean an acceptable degree of error for the quantity measured given the nature or precision of the measurements. Typical, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

The term “molecule” means any distinct or distinguishable structural unit of matter comprising one or more atoms, and includes, for example, polypeptides and polynucleotides.

The term “therapeutically effective dose” refers to that amount of a compound or compositions that is sufficient to result in a desired activity.

The phrase “pharmaceutically acceptable” refers to molecular entities and compositions that are physiologically tolerable and do not typically produce an allergic or similar untoward reaction (for example, gastric upset, dizziness and the like) when administered to an individual. Preferably, and particularly where a vaccine is used in humans, the term “pharmaceutically acceptable” may mean approved by a regulatory agency (for example, the U.S. Food and Drug Agency) or listed in a generally recognized pharmacopeia for use in animals (for example, the U.S. Pharmacopeia).

The term “carrier” refers to a diluent, adjuvant, excipient, or vehicle with which a compound is administered. Sterile water or aqueous saline solutions and aqueous dextrose and glycerol solutions are preferably employed as carriers, particularly for injectable solutions. Exemplary suitable pharmaceutical carriers are described in “Reminington's Pharmaceutical Sciences” by E. W. Martin.

Molecular Biology Definitions. In accordance with the present invention, there may be employed conventional molecular biology, microbiology and recombinant DNA techniques within the skill of the art. Such techniques are explained fully in the literature. See, for example, Sambrook, Fitsch & Maniatis, Molecular Cloning: A Laboratory Manual, Second Edition (1989) Cold Spring Harbor Laboratory Press, Cold Spring Harbor, N.Y. (referred to herein as “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins, eds. 1984); Animal Cell Culture (R. I. Freshney, ed. 1986); Immobilized Cells and Enzymes (IRL Press, 1986); B. E. Perbal, A Practical Guide to Molecular Cloning (1984); F. M. Ausubel et al. (eds.), Current Protocols in Molecular Biology, John Wiley & Sons, Inc. (1994).

The term “polymer” means any substance or compound that is composed of two or more building blocks (‘mers’) that are repetitively linked together. For example, a “dimer” is a compound in which two building blocks have been joined togther; a “trimer” is a compound in which three building blocks have been joined together; etc.

The term “polynucleotide” or “nucleic acid molecule” as used herein refers to a polymeric molecule having a backbone that supports bases capable of hydrogen bonding to typical polynucleotides, wherein the polymer backbone presents the bases in a manner to permit such hydrogen bonding in a specific fashion between the polymeric molecule and a typical polynucleotide (e.g., single-stranded DNA). Such bases are typically inosine, adenosine, guanosine, cytosine, uracil and thymidine. Polymeric molecules include “double stranded” and “single stranded” DNA and RNA, as well as backbone modifications thereof (for example, methylphosphonate linkages).

Thus, a “polynucleotide” or “nucleic acid” sequence is a series of nucleotide bases (also called “nucleotides”), generally in DNA and RNA, and means any chain of two or more nucleotides. A nucleotide sequence frequently carries genetic information, including the information used by cellular machinery to make proteins and enzymes. The terms include genomic DNA, cDNA, RNA, any synthetic and genetically manipulated polynucleotide, and both sense and antisense polynucleotides. This includes single- and double-stranded molecules; i.e., DNA-DNA, DNA-RNA, and RNA-RNA hybrids as well as “protein nucleic acids” (PNA) formed by conjugating bases to an amino acid backbone. This also includes nucleic acids containing modified bases, for example, thio-uracil, thio-guanine and fluoro-uracil.

The polynucleotides herein may be flanked by natural regulatory sequences, or may be associated with heterologous sequences, including promoters, enhancers, response elements, signal sequences, polyadenylation sequences, introns, 5′- and 3′-non-coding regions and the like. The nucleic acids may also be modified by many means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.). Polynucleotides may contain one or more additional covalently linked moieties, such as proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.) and alkylators to name a few. The polynucleotides may be derivatized by formation of a methyl or ethyl phosphotriester or an alkyl phosphoramidite linkage. Furthermore, the polynucleotides herein may also be modified with a label capable of providing a detectable signal, either directly or indirectly. Exemplary labels include radioisotopes, fluorescent molecules, biotin and the like. Other non-limiting examples of modification which may be made are provided, below, in the description of the present invention.

A “polypeptide” is a chain of chemical building blocks called amino acids that are linked together by chemical bonds called “peptide bonds”. The term “protein” refers to polypeptides that contain the amino acid residues encoded by a gene or by a nucleic acid molecule (e.g., an mRNA or a cDNA) transcribed from that gene either directly or indirectly. Optionally, a protein may lack certain amino acid residues that are encoded by a gene or by an mRNA. For example, a gene or mRNA molecule may encode a sequence of amino acid residues on the N-terminus of a protein (i.e., a signal sequence) that is cleaved from, and therefore may not be part of, the final protein. A protein or polypeptide, including an enzyme, may be a “native” or “wild-type”, meaning that it occurs in nature; or it may be a “mutant”, “variant” or “modified”, meaning that it has been made, altered, derived, or is in some way different or changed from a native protein or from another mutant.

A “ligand” is, broadly speaking, any molecule that binds to another molecule. In preferred embodiments, the ligand is either a soluble molecule or the smaller of the two molecule or both. The other molecule is referred to as a “receptor”. In preferred embodiments, both a ligand and its receptor are molecules (preferably proteins or polypeptides) produced by cells. Preferably, a ligand is a soluble molecule and the receptor is an integral membrane protein (i.e., a protein expressed on the surface of a cell). In a particularly preferred embodiment of the invention the ligand is a fibroblast growth factor (FGF) and the receptor is a fibroblast growth factor receptor (FGFR).

The binding of a ligand to its receptor is frequently a step of signal transduction with a cell. For example, in preferred embodiments where a ligand is an FGF polypeptide and a receptor is an FGFR polypeptide, the binding of FGF to the FGFR polypeptide may lead to activation of a tyrosine kinase activity within the FGFR polypeptide. Activation of the tyrosine kinase activity may, in turn, initiate other activities associated with FGF signaling, including but not limited to mitogenesis and angiogensis. Other exemplary ligand-receptor interactions include, but are not limited to, binding of a hormone to a hormone receptor (for example, the binding of estrogen to the estrogen receptor) and the binding of a neurotransmitter to a receptor on the surface of a neuron.

“Amplification” of a polynucleotide, as used herein, denotes the use of polymerase chain reaction (PCR) to increase the concentration of a particular DNA sequence within a mixture of DNA sequences. For a description of PCR see Saiki et al., Science 1988, 239:487.

“Chemical sequencing” of DNA denotes methods such as that of Maxam and Gilbert (Maxam-Gilbert sequencing; see Maxam & Gilbert, Proc. Natl. Acad. Sci. U.S.A. 1977, 74:560), in which DNA is cleaved using individual base-specific reactions.

“Enzymatic sequencing” of DNA denotes methods such as that of Sanger (Sanger et al., Proc. Natl. Acad. Sci. U.S.A. 1977, 74:5463) and variations thereof well known in the art, in a single-stranded DNA is copied and randomly terminated using DNA polymerase.

A “gene” is a sequence of nucleotides which code for a functional “gene product”. Generally, a gene product is a functional protein. However, a gene product can also be another type of molecule in a cell, such as an RNA (e.g., a tRNA or a rRNA). For the purposes of the present invention, a gene product also refers to an mRNA sequence which may be found in a cell. For example, measuring gene expression levels according to the invention may correspond to measuring mRNA levels. A gene may also comprise regulatory (i.e., non-coding) sequences as well as coding sequences. Exemplary regulatory sequences include promoter sequences, which determine, for example, the conditions under which the gene is expressed. The transcribed region of the gene may also include untranslated regions including introns, a 5′-untranslated region (5′-UTR) and a 3′-untranslated region (3′-UTR).

A “coding sequence” or a sequence “encoding” an expression product, such as a RNA, polypeptide, protein or enzyme, is a nucleotide sequence that, when expressed, results in the production of that RNA, polypeptide, protein or enzyme; i.e., the nucleotide sequence “encodes” that RNA or it encodes the amino acid sequence for that polypeptide, protein or enzyme.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently found, for example, by mapping with nuclease S1), as well as protein binding domains (consensus sequences) responsible for the binding of RNA polymerase.

A coding sequence is “under the control of” or is “operatively associated with” transcriptional and translational control sequences in a cell when RNA polymerase transcribes the coding sequence into RNA, which is then trans-RNA spliced (if it contains introns) and, if the sequence encodes a protein, is translated into that protein.

The term “express” and “expression” means allowing or causing the information in a gene or DNA sequence to become manifest, for example producing RNA (such as rRNA or mRNA) or a protein by activating the cellular functions involved in transcription and translation of a corresponding gene or DNA sequence. A DNA sequence is expressed by a cell to form an “expression product” such as an RNA (e.g., a mRNA or a rRNA) or a protein. The expression product itself, e.g., the resulting RNA or protein, may also said to be “expressed” by the cell.

The term “transfection” means the introduction of a foreign nucleic acid into a cell. The term “transformation” means the introduction of a “foreign” (i.e., extrinsic or extracellular) gene, DNA or RNA sequence into a host cell so that the host cell will express the introduced gene or sequence to produce a desired substance, in this invention typically an RNA coded by the introduced gene or sequence, but also a protein or an enzyme coded by the introduced gene or sequence. The introduced gene or sequence may also be called a “cloned” or “foreign” gene or sequence, may include regulatory or control sequences (e.g., start, stop, promoter, signal, secretion or other sequences used by a cell's genetic machinery). The gene or sequence may include nonfunctional sequences or sequences with no known function. A host cell that receives and expresses introduced DNA or RNA has been “transformed” and is a “transformant” or a “clone”. The DNA or RNA introduced to a host cell can come from any source, including cells of the same genus or species as the host cell or cells of a different genus or species.

The terms “vector”, “cloning vector” and “expression vector” mean the vehicle by which a DNA or RNA sequence (e.g., a foreign gene) can be introduced into a host cell so as to transform the host and promote expression (e.g., transcription and translation) of the introduced sequence. Vectors may include plasmids, phages, viruses, etc. and are discussed in greater detail below.

A “cassette” refers to a DNA coding sequence or segment of DNA that codes for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct.” A common type of vector is a “plasmid”, which generally is a self-contained molecule of double-stranded DNA, usually of bacterial origin, that can readily accept additional (foreign) DNA and which can readily introduced into a suitable host cell. A large number of vectors, including plasmid and fungal vectors, have been described for replication and/or expression in a variety of eukaryotic and prokaryotic hosts. The term “host cell” means any cell of any organism that is selected, modified, transformed, grown or used or manipulated in any way for the production of a substance by the cell. For example, a host cell may be one that is manipulated to express a particular gene, a DNA or RNA sequence, a protein or an enzyme. Host cells can further be used for screening or other assays that are described infra. Host cells may be cultured in vitro or one or more cells in a non-human animal (e.g., a transgenic animal or a transiently transfected animal).

The term “expression system” means a host cell and compatible vector under suitable conditions, e.g. for the expression of a protein coded for by foreign DNA carried by the vector and introduced to the host cell. Common expression systems include E. coli host cells and plasmid vectors, insect host cells such as Sf9, Hi5 or S2 cells and Baculovirus vectors, Drosophila cells (Schneider cells) and expression systems, and mammalian host cells and vectors.

The term “heterologous” refers to a combination of elements not naturally occurring. For example, the present invention includes chimeric RNA molecules that comprise an rRNA sequence and a heterologous RNA sequence which is not part of the rRNA sequence. In this context, the heterologous RNA sequence refers to an RNA sequence that is not naturally located within the ribosomal RNA sequence. Alternatively, the heterologous RNA sequence may be naturally located within the ribosomal RNA sequence, but is found at a location in the rRNA sequence where it does not naturally occur. As another example, heterologous DNA refers to DNA that is not naturally located in the cell, or in a chromosomal site of the cell. Preferably, heterologous DNA includes a gene foreign to the cell. A heterologous expression regulatory element is a regulatory element operatively associated with a different gene that the one it is operatively associated with in nature.

The terms “mutant” and “mutation” mean any detectable change in genetic material, e.g., DNA, or any process, mechanism or result of such a change. This includes gene mutations, in which the structure (e.g., DNA sequence) of a gene is altered, any gene or DNA arising from any mutation process, and any expression product (e.g., RNA, protein or enzyme) expressed by a modified gene or DNA sequence. The term “variant” may also be used to indicate a modified or altered gene, DNA sequence, RNA, enzyme, cell, etc.; i.e., any kind of mutant. For example, the present invention relates to altered or “chimeric” RNA molecules that comprise an rRNA sequence that is altered by inserting a heterologous RNA sequence that is not naturally part of that sequence or is not naturally located at the position of that rRNA sequence. Such chimeric RNA sequences, as well as DNA and genes that encode them, are also referred to herein as “mutant” sequences.

“Sequence-conservative variants” of a polynucleotide sequence are those in which a change of one or more nucleotides in a given codon position results in no alteration in the amino acid encoded at that position.

“Function-conservative variants” of a polypeptide or polynucleotide are those in which a given amino acid residue in the polypeptide, or the amino acid residue encoded by a codon of the polynucleotide, has been changed or altered without altering the overall conformation and function of the polypeptide. For example, function-conservative variants may include, but are not limited to, replacement of an amino acid with one having similar properties (for example, polarity, hydrogen bonding potential, acidic, basic, hydrophobic, aromatic and the like). Amino acid residues with similar properties are well known in the art. For example, the amino acid residues arginine, histidine and lysine are hydrophilic, basic amino acid residues and may therefore be interchangeable. Similar, the amino acid residue isoleucine, which is a hydrophobic amino acid residue, may be replaced with leucine, methionine or valine. Such changes are expected to have little or no effect on the apparent molecular weight or isoelectric point of the polypeptide. Amino acid residues other than those indicated as conserved may also differ in a protein or enzyme so that the percent protein or amino acid sequence similarity (e.g., percent identity or homology) between any two proteins of similar function may vary and may be, for example, from 70% to 99% as determined according to an alignment scheme such as the Cluster Method, wherein similarity is based on the MEGALIGN algorithm. “Function-conservative variants” of a given polypeptide also include polypeptides that have at least 60% amino acid sequence identity to the given polypeptide as determined, e.g., by the BLAST or FASTA algorithms. Preferably, function-conservative variants of a given polypeptide have at least 75%, more preferably at least 85% and still more preferably at least 90% amino acid sequence identity to the given polypeptide and, preferably, also have the same or substantially similar properties (e.g., of molecular weight and/or isoelectric point) or functions (e.g., biological functions or activities) as the native or parent polypeptide to which it is compared.

Thus, for example, in particular embodiments wherein the polypeptides are FGFR polypeptides, function-conservative variants may not only have between at least 75% and at least 90% amino acid sequence identity to a given FGFR, but preferably also have similar properties, such as conserved domains (e.g., as in a D1, D2 or D3 domain, described supra) and/or similar biological function or activities, such as a tyrosine kinase activity and/or the ability to stimulate activities associated with FGF signaling (e.g., mitogenesis or angiogenesis).

Similarly, in embodiments wherein a polypeptide is an FGF ligand, function-conservative variants may not only have between at least 75% and at least 90% amino acid sequence identity to a given FGF, but preferably also have similar properties. For example, a function-conservative variant of an FGF ligand preferably binds to the same FGF receptor as the FGF ligand (preferably, but not necessarily with the same or a similar affinity; e.g., preferably with at least 50% of the binding affinity, more preferably with at least 70% of the binding affinity, and still more preferably with at least 80% or at least 90% of the binding affinity). Preferably, by binding to the FGFR polypeptide, a function-conservative variant will also stimulate a same biological function or activity that is associated with binding of the FGF ligand to the receptor, including any of the functions or activities described, supra, for an FGF receptor.

The term “homologous”, in all its grammatical forms and spelling variations, refers to the relationship between two proteins that possess a “common evolutionary origin”, including proteins from superfamilies (e.g., the immunoglobulin superfamily) in the same species of organism, as well as homologous proteins from different species of organism (for example, myosin light chain polypeptide, etc.; see, Reeck et al., Cell 1987, 50:667). Homologous proteins of the invention therefore include various FGF proteins and polypeptides derived from the same species of organism (i.e., the FGF family of polypeptides, including FGF1-FGF22), and also FGF proteins and polypeptides derived from different species of organisms. Similarly, homologous proteins of the invention also include various FGFR proteins and polypeptides derived from the same species (i.e., the FGFR family, including FGFR1-4) or from different species of organisms.

Such proteins (and their encoding nucleic acids) have sequence homology, as reflected by their sequence similarity, whether in terms of percent identity or by the presence of specific residues or motifs and conserved positions. For instance, referring again to particular embodiments where homologous polypeptides are FGF and/or FGFR polypeptides, homologous polypeptides in either the same or in closely related species of organisms (for example, between mammals such as mice and humans) typically share greater than 50% sequence identity, more preferably share at least about 60 to 65% sequence identity, and still more preferably share at least about 75% to 80% sequence identity. Homologous polypeptides between closely related species of organisms may also be cross reactive in both species of organisms. For example, an FGF from one species of organism may bind to and/or activate an FGF receptor polypeptide from a different species of organism and, moreover, an FGF receptor from a first species of organism may stimulate a activity associated with FGF signalling (e.g., mitogenesis or angiogenesis) in a cell from a different species of organism (for example, when the heterologous FGFR polypeptide is recombinantly expressed in that cell).

By contrast, FGF and/or FGFR polypeptides between more divergent species of organisms share less sequence identity and generally are not cross reactive in both species. For example, homologous polypeptides between divergent species of organisms typically share less than 50% sequence identity, and may share only 25% sequence identity. However, homologous polypeptides between divergent species preferably share a higher level of sequence identity, such as between about 35% to 45% sequence identity.

The term “sequence similarity”, in all its grammatical forms, refers to the degree of identity or correspondence between nucleic acid or amino acid sequences that may or may not share a common evolutionary origin (see, Reeck et al., Cell 1987, 50:667). However, in common usage and in the instant application, the term “homologous”, particularly when modified with an adverb such as “highly”, may refer to sequence similarity and may or may not relate to a common evolutionary origin.

In specific embodiments, two nucleic acid sequences are “substantially homologous” or “substantially similar” when at least about 80%, and more preferably at least about 90% or at least about 95% of the nucleotides match over a defined length of the nucleic acid sequences, as determined by a sequence comparison algorithm known such as BLAST, FASTA, DNA Strider, CLUSTAL, etc. An example of such a sequence is an allelic or species variant of the specific genes of the present invention. Sequences that are substantially homologous may also be identified by hybridization, e.g., in a Southern hybridization experiment under, e.g., stringent conditions as defined for that particular system.

Similarly, in particular embodiments of the invention, two amino acid sequences are “substantially homologous” or “substantially similar” when greater than 80% of the amino acid residues are identical, or when greater than about 90% of the amino acid residues are similar (i.e., are functionally identical). Preferably the similar or homologous polypeptide sequences are identified by alignment using, for example, the GCG (Genetics Computer Group, Program Manual for the GCG Package, Version 7, Madison Wis.) pileup program, or using any of the programs and algorithms described above (e.g., BLAST, FASTA, CLUSTAL, etc.).

As used herein, the term “oligonucleotide” refers to a nucleic acid, generally of at least 10, preferably at least 15, and more preferably at least 20 nucleotides, preferably no more than 100 nucleotides, that is hybridizable to a genomic DNA molecule, a cDNA molecule, or an mRNA molecule encoding a gene, mRNA, cDNA, or other nucleic acid of interest. Oligonucleotides can be labeled, e.g., with ³²P-nucleotides or nucleotides to which a label, such as biotin or a fluorescent dye (for example, Cy3 or Cy5) has been covalently conjugated. In one embodiment, a labeled oligonucleotide can be used as a probe to detect the presence of a nucleic acid. In another embodiment, oligonucleotides (one or both of which may be labeled) can be used as PCR primers; e.g. for cloning full length or a fragment of either an FGF or an FGFR nucleic acid, or to detect the presence of nucleic acids encoding either an FGF or an FGFR polypeptide. Generally, oligonucleotides are prepared synthetically, preferably on a nucleic acid synthesizer. Accordingly, oligonucleotides can be prepared with non-naturally occurring phosphoester analog bonds, such as thioester bonds, etc.

Specific non-limiting examples of synthetic oligonucleotides envisioned for this invention include, in addition to the nucleic acid moieties described above, oligonucleotides that contain phosphorothioates, phosphotriesters, methyl phosphonates, short chain alkyl, or cycloalkyl intersugar linkages or short chain heteroatomic or heterocyclic intersugar linkages. Most preferred are those with CH₂—NH—O—CH₂, CH₂—N(CH₃)—O—CH₂, CH₂—O—N(CH₃)—CH₂, CH₂—N(CH₃)—N(CH₃)—CH₂ and O—N(CH₃)—CH₂—CH₂ backbones (where phosphodiester is O—PO₂—O—CH₂). U.S. Pat. No. 5,677,437 describes heteroaromatic olignucleoside linkages. Nitrogen linkers or groups containing nitrogen can also be used to prepare oligonucleotide mimics (U.S. Pat. Nos. 5,792,844 and 5,783,682). U.S. Pat. No. 5,637,684 describes phosphoramidate and phosphorothioamidate oligomeric compounds. Also envisioned are oligonucleotides having morpholino backbone structures (U.S. Pat. No. 5,034,506). In other embodiments, such as the peptide-nucleic acid (PNA) backbone, the phosphodiester backbone of the oligonucleotide may be replaced with a polyamide backbone, the bases being bound directly or indirectly to the aza nitrogen atoms of the polyamide backbone (Nielsen et al., Science 254:1497, 1991). Other synthetic oligonucleotides may contain substituted sugar moieties comprising one of the following at the 2′ position: OH, SH, SCH₃, F, OCN, O(CH₂)ₙNH₂ or O(CH₂)ₙCH₃ where n is from 1 to about 10; C, to C₁₀ lower alkyl, substituted lower alkyl, alkaryl or aralkyl; Cl; Br; CN; CF₃; OCF₃; O—; S—, or N-alkyl; O-, S-, or N-alkenyl; SOCH₃; SO₂CH₃; ONO₂; NO₂; N₃; NH₂; heterocycloalkyl; heterocycloalkaryl; aminoalkylamino; polyalkylamino; substitued silyl; a fluorescein moiety; an RNA cleaving group; a reporter group; an intercalator; a group for improving the pharmacokinetic properties of an oligonucleotide; or a group for improving the pharmacodynamic properties of an oligonucleotide, and other substituents having similar properties. Oligonucleotides may also have sugar mimetics such as cyclobutyls or other carbocyclics in place of the pentofuranosyl group. Nucleotide units having nucleosides other than adenosine, cytidine, guanosine, thymidine and uridine, such as inosine, may be used in an oligonucleotide molecule.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook et al., supra). The conditions of temperature and ionic strength determine the “stringency” of the hybridization. For preliminary screening for homologous nucleic acids, low stringency hybridization conditions, corresponding to a Tₘ (melting temperature) of 55° C., can be used, e.g., 5×SSC, 0.1% SDS, 0.25% milk, and no formamide; or 30% formamide, 5×SSC, 0.5% SDS). Moderate stringency hybridization conditions correspond to a higher Tₘ, e.g., 40% formamide, with 5× or 6×SCC. High stringency hybridization conditions correspond to the highest Tₘ, e.g., 50% formamide, 5× or 6×SCC. SCC is a 0.15M NaCl, 0.015M Na-citrate. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tₘ for hybrids of nucleic acids having those sequences. The relative stability (corresponding to higher Tₘ) of nucleic acid hybridizations decreases in the following order: RNA:RNA, DNA:RNA, DNA:DNA. For hybrids of greater than 100 nucleotides in length, equations for calculating Tₘ have been derived (see Sambrook et al., supra, 9.50-9.51). For hybridization with shorter nucleic acids, i.e., oligonucleotides, the position of mismatches becomes more important, and the length of the oligonucleotide determines its specificity (see Sambrook et al., supra, 11.7-11.8). A minimum length for a hybridizable nucleic acid is at least about 10 nucleotides; preferably at least about 15 nucleotides; and more preferably the length is at least about 20 nucleotides.

In a specific embodiment, the term “standard hybridization conditions” refers to a Tₘ of 55° C., and utilizes conditions as set forth above. In a preferred embodiment, the Tₘ is 60° C.; in a more preferred embodiment, the Tₘ is 65° C. In a specific embodiment, “high stringency” refers to hybridization and/or washing conditions at 68° C. in 0.2×SSC, at 42° C. in 50% formamide, 4×SSC, or under conditions that afford levels of hybridization equivalent to those observed under either of these two conditions.

Suitable hybridization conditions for oligonucleotides (e.g., for oligonucleotide probes or primers) are typically somewhat different than for full-length nucleic acids (e.g., full-length cDNA), because of the oligonucleotides' lower melting temperature. Because the melting temperature of oligonucleotides will depend on the length of the oligonucleotide sequences involved, suitable hybridization temperatures will vary depending upon the oligoncucleotide molecules used. Exemplary temperatures may be 37° C. (for 14-base oligonucleotides), 48° C. (for 17-base oligoncucleotides), 55° C. (for 20-base oligonucleotides) and 60° C. (for 23-base oligonucleotides). Exemplary suitable hybridization conditions for oligonucleotides include washing in 6×SSC/0.05% sodium pyrophosphate, or other conditions that afford equivalent levels of hybridization.

X-ray crystallography. The present invention also uses techniques of conventional X-ray crystallography. These techniques are well known and are within the routine skill of the art. Such techniques are described more fully in the literature. See, for example, Cantor&Schimmel, Biophysical Chemistry 1980 (Vols. I-E1) W. H. Freeman and Company (particularly Chapters 1-13 in Vol. 1, and Chapter 13 in Vol. I). See, also, Macromolecular Crystallography, Parts A-B (Carter&Sweet, Eds.) In: Methods Enzymol. 1997, Vols. 276-277; Jan Drenth, Principles of Protein X-Ray Crystallography (New York: Springer-Verlag, 1994).

The term “crystal” refers, generally, to any ordered (or at least partially ordered) three-dimensional array of molecules. Preferably, the ordering of molecules within a crystal is at least sufficient to produce a sharp X-ray diffraction pattern so that the molecules' three-dimensional structure may be determined.

The molecules in a crystal may be of any type, and it will be understood that a crystal may contain molecules of only one type or may comprise a plurality of different types of molecules. In preferred embodiments, crystals of the present invention comprise at least one biomolecule, such as a protein, or a fragment thereof. Crystals of the invention may even comprise a complex or assembly of two or more proteins or other biomolecules. For example, a crystal may comprise two different proteins, such as a receptor and a ligand, or a crystal may comprise two more molecules of the same protein bound together, e.g., to form a dimer or other multimer complex. Typically, crystals that contain biological molecules such as proteins will contain other molecules as well, such molecules of solvent (e.g., water molecules) and/or salt. Other molecules such as drugs, drug candidates or compounds that bind to the protein may also be present in a crystal.

It will be understood by a skilled artisan that crystals of the invention comprises a “unit cell”, or basic parallelepiped shaped block defined by vectors denoted a, b and c. The entire volume of a crystal may be constructed by the regular assembly of such blocks or “lattices”. A crystal is also defined by the overall symmetry of elements (i.e., molecules) within the cell, which is referred to as the “space group.” Thus, a crystal's space group is defined by symmetry relations within the molecules making up the unit cell. The “asymmetric unit” is the smallest possible unit from which the crystal structure may be generated by making use of the symmetric relations defining the space group.

The term “structure coordinates” or “structure” refers to mathematical coordinates that define the position of atoms in a molecule or in an assembly of molecules in three-dimensional space (for example, within the asymmetric unit of a crystal). Structure coordinates may be computed or otherwise determined using any information related to the three dimensional arrangement of atoms in a molecule. However, in preferred embodiments of the invention a structure is derived from equations that are related to patterns obtained on diffraction of a monochromatic beam of X-rays by the atoms (which, in such embodiments, may also be referred to as “scattering centers”) in a crystal. Typically, such diffraction data is used to calculate an “electron density” map of the crystal's asymmetric unit, and these maps are used, in turn, to establish positions of the individual atoms.

“Heavy atom derivatization” refers to a method of producing chemically modified forms of a crystal (typically a crystal of a protein or other biopolymer), in which the crystal may be soaked in a solution containing heavy metal atom salts or organometallic compounds that can diffuse through the crystal and bind to the surface of the protein or biopolymer. The location(s) of one or more heavy meatl atoms in the crystal may then be determined by X-ray diffraction analysis of the soaked crystal, and this information may be used to facilitate construction of the three-dimension structure of the protein or other molecules contained in the crystal.

“Molecular replacement” refers to a method wherein a preliminary structure coordinates are generated for molecules in a crystal whose structure coordinates are not known. Generally, molecular replacement involves orienting and/or positioning another, preferably similar molecule (such as a homologous protein) whose structure coordinates are known. Phases for an X-ray diffraction pattern may then be determined for the preliminary structure, and these phases can then be combined with actual X-ray diffraction intensities that are observed for the crystal whose structure coordinates are not known, to determine its structure.

### FGF Ligands

FGF Polypeptides. The present invention relates to polypeptides known as fibroblast growth factor (FGF) polypeptides or, alternatively, as FGF ligands. FGF polypeptides are well known in the art and have been described, e.g., by Mckeehan et al., (Progress in Nucleic Acid Research and Molecular Biology 1998, 59:135-176). See, also, Nishimura et al., Biochim. Biophys. Acta 2000, 1492:203-206; and Yamashita et al., Biochem. Biophys. Res. Commun. 2000, 277:494-498. Structurally, all FGF's share a common core domain consisting of about 120 amino acids, which fold into three copies of four-stranded β-sheets known as a β-trefoil fold.

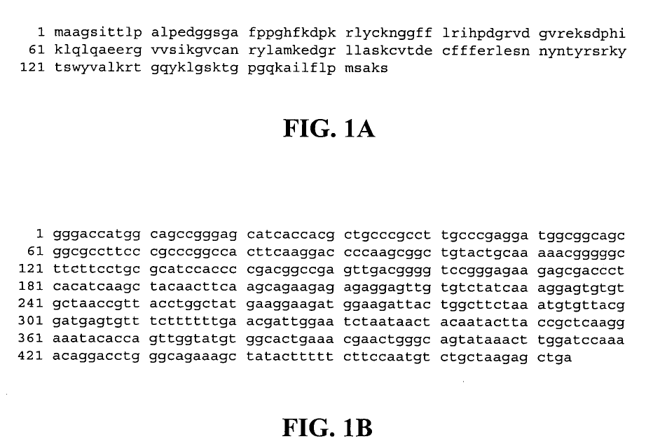

The amino acid sequence of one, exemplary FGF polypeptide, known as FGF2, is set forth here in FIG. 1A and in SEQ ID NO:1. The FGF2 polypeptide sequence is also available from GenBank and has the Accession No. P09038 (GI:122742). The β-trefoil domain corresponds to approximately amino acid residues 28-152 of this FGF2 polypeptide sequence. The FGF2 amino acid sequence shown in FIG. 1A (SEQ ID NO:1) represents the “pre-cursor” form of the FGF2 polypeptide. This precursor is ordinarily processed by the cell and secreted as a “mature” FGF2 polypeptide comprising amino acid residues 10-155 of SEQ ID NO:1.

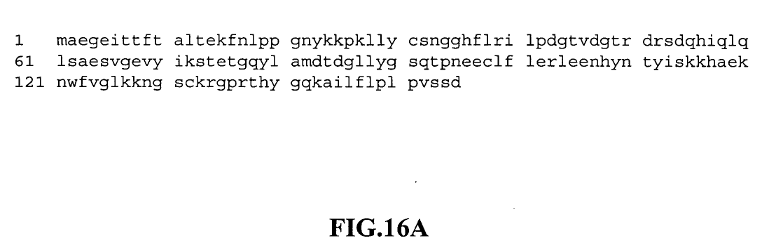

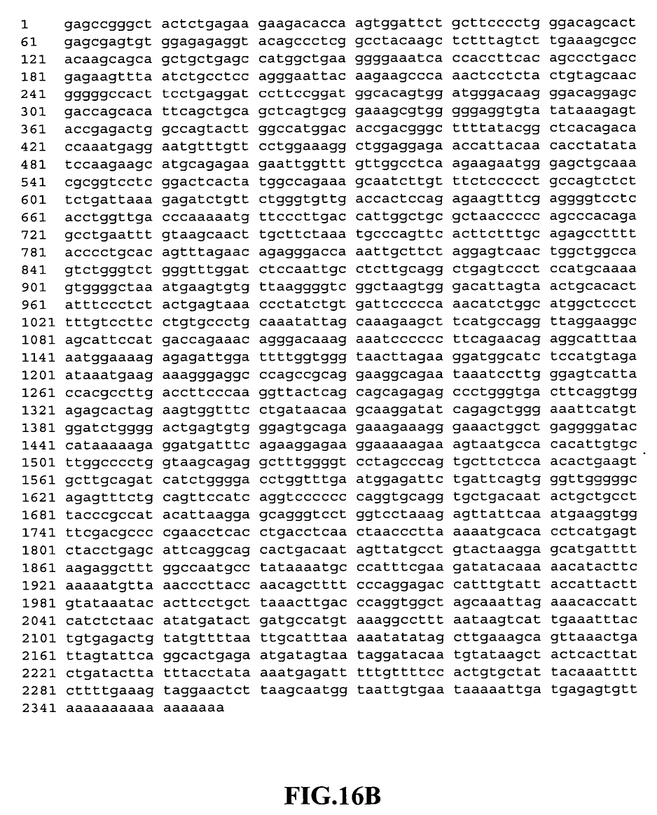

The amino acid sequence of a second exemplary FGF polypeptide known as FGF1 is also set forth here, in FIG. 16A and in SEQ ID NO:5. The FGF1 polypeptide is also known in the art as the acidic FGF or “aFGF”, and its sequence is available from GenBank under the Accession No. NP_{—}000791 (GI:4503697). The FGF1 amino acid sequence shown in FIG. 16A (SEQ ID NO:5) represents the “pre-cursor” form of the FGF1 polypeptide. This precursor is ordinarily processed by the cell and secreted as a “mature” FGF1 polypeptide comprising amino acid residues 16-155 of SEQ ID NO:5

Numerous variants, including FGF homologs and orthologs from the same and different species of organisms are also known in the art and/or may be readily identified. Such variants may also be used in the methods and compositions of this invention. For example, at least 22 homologous human FGF polypeptides, referred to as FGF1-FGF22, are believed to exist. The FGF polypeptides of the invention therefore include each of these human homologs, and also include homologous or orthologous polypeptides isolated from other species of organisms, particularly other mammalian species such as mouse or rat. Sequences that are substantially homologous to known FGF polypeptide sequences (e.g., to the FGF2 sequence shown in FIG. 1A and in SEQ ID NO:1 or to the FGF1 sequence in FIG. 16A and in SEQ ID NO:5) can be readily identified by comparing the sequences using standard software packages available in sequence data banks, including the BLAST algorithms (e.g., BLASTP, BLASTN, BLASTX, etc.), FASTA, DNA Strider, the GCG pileup program, CLUSTAL and other such programs that are known in the art or are described herein.

Thus, for example, FGF polypeptides of the invention also include ones encoded by nucleic acids that hybridize to the complement of a nucleic acid molecule encoding an FGF polypeptide (e.g., in a Southern hybridization experiment under defined conditions). For example, in particular embodiments an FGF polypeptide may comprise an amino acid sequence encoded by nucleic acid molecules that hybridize to the complement of an FGF2 nucleic acid sequence, such as the coding sequence set forth in FIG. 1B (SEQ ID NO:2), under highly stringent conditions that comprise 50% formamide in 5× or 6×SSC. In other embodiments, the FGF polypeptide may comprise an amino acid sequence encoded by nucleic acid molecules that hybridize to a complement of an FGF2 nucleic acid sequence (e.g., the coding sequence in FIG. 1B and SEQ ID NO:2) under moderately stringent hybridization conditions (for example, 40% formamide with 5× or 6×SSC), or under low stringency conditions (for example, in 5×SSC, 0.1% SDS, 0.25% milk, no formamide, 30% formamide, 5×SSC, or 0.5% SDS). Similarly, FGF polypeptides of the invention also encompass ones encoded by nucleic acids that hybridize to the complement of an FGF1 nucleic acid sequence, such as the coding sequence set forth in FIG. 16B (SEQ ID NO:6) under the same conditions.

In still other embodiments, FGF polypeptides can also be identified by isolating homologous or variant FGF genes, e.g., by PCR using degenerate oligonucleotide primers designed on the basis of a given FGF polypeptide sequence and as described below.

FGF polypeptides of the invention also include polypeptides that comprise one or more partial or fragment FGF amino acid sequences; i.e. a portion or fragment of a full length FGF amino acid sequence such as the full length FGF2 sequence shown in FIG. 1A (SEQ ID NO:1) or, alternatively, a portion or fragment of the full length FGF1 sequence shown in FIG. 16A (SEQ ID NO:5). Such partial FGF polypeptides may comprise, for example, an amino acid sequence of one or more epitopes or domains of a full length FGF polypeptide, such as epitopes or domains of a full length FGF2 polypeptide set forth in FIG. 1B (SEQ ID NO:2) or, alternatively, of a full length FGF1 polypeptide set forth in FIG. 16A (SEQ ID NO:5). An epitope of an FGF polypeptide represents a site on the polypeptide against which an antibody may be produced and to which the antibody binds. Therefore, polypeptides comprising the amino acid sequence of an FGF epitope are useful for making antibodies to the FGF polypeptide. Preferably, an epitope comprises a sequence of at least 5, more preferably at least 10, 15, 20, 25 or 50 amino acid residues in length. Thus, polypeptides of the invention that comprise epitopes of an FGF polypeptide preferably contain an amino acid sequence corresponding to at least 5, at least 10, at least 15, at least 20, at least 25 or at least 50 amino acid residues of a full length FGF polypeptide sequence. For example, in certain preferred embodiments wherein the epitope is an epitope of a full length FGF2 polypeptide (SEQ ID NO:1), an FGF polypeptide of the invention preferably comprises an amino acid sequence corresponding to at least 5, at least 10, at least 15, at least 20, at least 25 or at least 50 amino acid residues of the FGF2 sequence set forth in FIG. 1A (SEQ ID NO:1). Similarly, in embodiments where the epitope is an epitope to a full length FGF1 polypeptide (SEQ ID NO:5), an FGF polypeptide of the invention can comprise an amino acid sequence corresponding to at least 5, at least 10, at least 15, at least 20, at least 25 or at least 50 amino acid residues of the FGF1 sequence set forth in FIG. 16A (SEQ ID NO:5).

Truncated forms of an FGF polypeptide can also be provided. Such truncated forms may include an FGF polypeptide with a specific deletion of amino acid residues. For instance, in certain embodiments amino acid residues corresponding to one or more domains of a full length FGF polypeptide may be deleted from the amino acid sequence of an FGF polypeptide.

The FGF polypeptides of this invention include, in addition to naturally occurring homologs and orthologs of an FGF polypeptide such as FGF2 (SEQ ID NO:1) and FGF1 (SEQ ID NO:5), but also include analogs and derivatives of an FGF polypeptide. Such analogs and derivatives may be ones that are naturally occurring (such as allelic variants), or may be man made (such as fusion proteins). However, analogs and derivatives of an FGF polypeptide of this invention will have the same or homologous characteristics of FGF polypeptides set forth above.

An FGF chimeric or fusion polypeptide may also be prepared in which the FGF portion of the fusion polypeptide has one or more characteristics of the FGF polypeptide. Such fusion polypeptides therefore represent alternative embodiments of the FGF polypeptides of this invention. Exemplary FGF fusion polypeptides include ones which comprise a full length, derivative or truncated FGF amino acid sequence, as well as fusions which comprise a fragment of an FGF polypeptide sequence (e.g., a fragment corresponding to an epitope or to one or more domains). Such fusion polypeptides may also comprise the amino acid sequence of a second, different polypeptide. For example, a fusion protein of the invention may comprise the amino acid sequence of a marker polypeptide; such as FLAG, a histidine tag, glutathione S-transferase (GST), or an Fc portion of an IgG. In other embodiments, an FGF polypeptide may be expressed with (e.g., fused to) a bacterial protein such as β-galactosidase. Additionally, FGF fusion polypeptides may comprise amino acid sequences that increase solubility of the polypeptide, such as thioreductase amino acid sequence, or the sequence of one or more immunoglobulin proteins (e.g., IgG1 or IgG2).

FGF analogs or variants can also be made by altering encoding nucleic acid molecules, for example by substitutions, additions or deletions. Preferably such altered nucleic acid molecules encode functionally similar molecules (i.e., molecules that perform one or more functions of an FGF ligand and/or have one or more FGF bioactivities). Thus, in a specific embodiment, an analog or variant of an FGF ligand is a function-conservative analog or variant.

Amino acid residues, other than ones that are specifically identified herein as being conserved, may differ among variants of a protein or polypeptide. Accordingly, the percentage of protein or amino acid sequence similarity between any two FGF polypeptides of similar function may vary. Typically, the percentage of protein or amino acid sequence similarity between different FGF variants may be from 70% to 99%, as determined according to an alignment scheme such as the Cluster Method and/or the MEGALIGN or GCG alignment algorithm. “Function-conservative variants” also include polypeptides that have greater than or at least 20%, or greater than or at least 25%, preferably greater than or at least 45%, more preferably greater than or at least 50, 75, 85, 90 or 95% sequence similarity to a FGF polypeptide (such as FGF2, set forth in SEQ ID NO:1 and in FIG. 1A; or, alternatively, FGF1 set forth in SEQ ID NO:5 and in FIG. 16A) or to one or more fragments or domains thereof. Preferably, such function-conservative variants also have the same or similar properties, functions or bioactivities as the native polypeptide to which they are compared. It is further noted that function-conservative variants of the present invention include, not only variants of a full length FGF polypeptide, but also include function-conservative variants of modified FGF polypeptides (e.g., truncations and deletions) and of fragments (e.g., corresponding to domains or epitopes) of full length FGF polypeptides.

In still other embodiments, an analog of an FGF polypeptide may be an allelic variant or mutant FGF polypeptide. The terms allelic variant and mutant, when used herein to describe a polypeptide, refer to a polypeptide encoded by an allelic variant or mutant gene. Thus, the allelic variant and mutant FGF polypeptides of the invention are polypeptides encoded by allelic variants or mutants of an FGF nucleic acid. (described infra).

FGF polypeptides of the invention also include derivative FGF polypeptides, which may be phosphorylated, myristylated, methylated or otherwise chemically modified. Such derivative FGF polypeptides also include labeled variants; for example, radio-labeled with iodine, phosphorous or sulfur (see, e.g., EP 372707 B) or FGF polypeptides labeled with other detetable molecules such as, but by no means limited to, biotin, a fluorescent dye (e.g., Cy5 or Cy3), a chelating group complexed with a metal ion, a chromophore or fluorophore, a gold colloid, a particular such as a latex bead, or attached to a water soluble polymer.

Chemical modifications of a biologically active component or components of FGF nucleic acids or polypeptides may provide additional advantages under certain circumstances. See, for example, U.S. Pat. No. 4,179,337 issued Dec. 18, 1970 to Davis et al. Also, for a review see, Abuchowski et al., in Enzymes as Drugs (J. S. Holcerberg & J. Roberts, eds.) 1981, pages 367-383. A review article describing protein modification and fusion proteins is also found in Fracis, Focus on Growth Factors 1992, 3:4-10, Mediscript: Mountview Court, Friem Bamet Lane, London N20, OLD, UK.

While the above, exemplary variants and analogs of FGF polypeptides are described primarily in terms of the exemplary FGF polypepide, FGF2 (set forth in FIG. 1A and SEQ ID NO:1) and FGF1 (set forth in FIG. 16A and SEQ ID NO:5), it is understood that variant FGF polypeptides of the invention include other FGF polypeptides (e.g., naturally occurring homologs and orthologs, described supra) having equivalent amino acid substitutions, deletions or insertions.

FGF nucleic acids. In general, an FGF nucleic acid molecule of the present invention comprises a nucleic acid sequence that encodes an FGF polypeptide (as defined, above, in this Subsection) or the complement of an FGF polypeptide encoding sequence. The invention also provides fragments of FGF encoding sequences and their complements, and such sequences are also considered part of the FGF nucleic acid molecules of this invention. Thus, in one exemplary embodiment, an FGF nucleic acid molecule of the invention may encode the exemplary FGF2 polypeptide sequence set forth in FIG. 1A (SEQ ID NO:1), such as the particular FGF2 nucleic acid sequence that is depicted in FIG. 1B (i.e., SEQ ID NO:2). In another exemplary embodiment, an FGF nucleic acid of the invention may encode the eemplary FGF1 polypeptide sequence set forth in FIG. 16A (SEQ ID NO:5), such as the particular FGF1 nucleic acid sequence shown in FIG. 16B (SEQ ID NO:6).

In still other embodiments, the FGF nucleic acid molecules of the invention comprise nucleic acid sequences that encode one or more domains of an FGF polypeptide.

The FGF nucleic acid molecules of the invention also include nucleic acids which comprise a sequence encoding one or more fragments of an FGF polypeptide. Such fragments include, for example, polynucleotides that encode an epitope of an FGF polypeptide; e.g., nucleic acids that encode a sequence of at least 5, and more preferably at least 10, 15, 20, 25 or 50 amino acid residues of an FGF polypeptide sequence (for example, of the exemplary FGF2 polypeptide sequence set forth in FIG. 1A and in SEQ ID NO:1 or, alternatively, of the exemplary FGF1 polypeptide sequence in FIG. 16A and in SEQ ID NO:5).

As explained above, numerous variant FGF polypeptides are known in the art and may be readily identified by those skilled in the art, including homologous and orthologous polypeptides from the same and different species of organism. The FGF nucleic acid molecules of the invention therefore include nucleic acid molecule comprising coding sequences for variant FGF polypeptides (including allelic variants, analogs and homologous from the same or different species), as well as nucleic acid molecule comprising coding sequences for modified FGF polypeptides (e.g., having amino acid substitutions, deletions or truncations). In preferred embodiments, such nucleic acid molecules have at least 50%, preferably at least 75% and more preferably at least 90% sequence identity to another FGF coding sequence, such as the exemplary FGF2 coding sequence set forth in FIG. 1B (SEQ ID NO:2) or, alternatively, the exemplary FGF1 coding sequence shown in FIG. 16B (SEQ ID NO:6).

In addition, the FGF nucleic acid molecules of the invention include nucleic acid molecules that hybridize to another FGF nucleic acid molecule, e.g., in a Southern blot assay under defined conditions. For example, in specific embodiments an FGF nucleic acid molecule of the invention comprises a nucleotide sequence which hybridizes to a complement of the exemplary FGF2 coding sequence set forth in FIG. 1B (SEQ ID NO:2) under highly stringent hybridization conditions that comprise 50% formamide and 5× or 6×SSC. In other embodiments, the nucleic acid molecules hybridize to a complement of an FGF nucleic acid sequence (e.g., to the exemplary coding sequence set forth in FIG. 1B and in SEQ ID NO:2) under moderately stringent hybridization conditions (e.g., in 5×SSC, 0.1% SDS, 0.25% milk, no formamide, 30% formamide, 5×SSC or 0.5% SDS). Similarly, an FGF nucleic acid of the invention may comprise a nucleotide sequence that hybridizes to a complement of the exemplary FGF1 coding sequence set forth in FIG. 16B (SEQ ID NO:6) under the same conditions. Alternatively, an FGF nucleic acid molecule may hybridize, under the same defined hybridization conditions, to the complement of a fragment of a nucleotide sequence encoding a full length FGF polypeptide.

In other embodiments, FGF nucleic acid molecules of the invention comprise fragments of a full length FGF nucleic acid sequence. Such nucleic acid fragments comprise a nucleotide sequence that corresponds to a sequence of at least 10 nucleotides, preferably at least 15 nucleotides and more preferably at least 20 nucleotides of a full length coding FGF nucleotide sequence. In specific embodiments, the fragments correspond to a portion (e.g., of at least 10, 15, or 20 nucleotides) of the exemplary FGF2 coding sequence shown in FIG. 1B (SEQ ID NO:2) or of the exemplary FGF1 coding sequence shown in FIG. 16B (SEQ ID NO:6). In other embodiments, an FGF nucleic acid fragment may comprise sequences of at least 10, preferably at least 15, and more preferably at least 20 nucleotides that are complementary and/or hybridize to a full length FGF coding sequence (e.g., the FGF2 coding sequence set forth in FIG. 1B and in SEQ ID NO:2, or the FGF1 coding sequence set forth in FIG. 16B and in SEQ ID NO:6) or to a fragment thereof.

Suitable hybridization conditions for such oligonucleotides are described supra, and include washing in 6×SSC/0.05% sodium pyrophosphate. Because the melting temperature of oligonucleotides will depend on the length of the oligonucleotide sequence, suitable hybridization temperatures may vary depending upon the oligonucleotide molecules used. Those skilled in the art will be able to select a suitable hybridization temperature using routine techniques described, e.g., in any of the molecular biology references cited supra. Exemplary temperatures will be 37° C. (e.g., for 14-base oligonucleotides), 48° C. (e.g., for 17-base oligonucleotides), 55° C. (e.g., for 20-base oligonucleotides) and 60° C. (e.g., for 23-base oligonucleotides).

Nucleic acid molecules comprising such fragments are useful, for example, as oligonucleotide probes and primers (e.g., PCR primers) to detect and amplify other nucleic acid molecules encoding an FGF polypeptide, including genes that encode variant FGF polypeptides (including genes that encode homologous or orthologous FGF polypeptides from the same or different species of organism). Oligonucleotide fragments of the invention may also be used, e.g., as antisense nucleic acids, triple helix forming oligonucleotides or as ribozymes (e.g., to modulate levels of FGF gene expression or transcription in cells).

The nucleic acid molecules of the invention also include “chimeric” FGF nucleic acid molecules. Such chimeric nucleic acid molecules are polynucleotides which comprise at least one FGF nucleic acid sequence (which may be any of the full length or partial FGF nucleic acid sequences described above), and also at least one non-FGF nucleic acid sequence. For example, the non-FGF nucleic acid sequence may be a heterologous regulatory sequence (for example, a promoter sequence) that is derived from another, non-FGF gene and is not normally associated with a naturally occurring FGF gene. A non-FGF nucleic acid sequence of the invention may also be a coding sequence of another, non-FGF polypeptide such as FLAG, a histidine tag, glutathione S-transferase (GST), hemaglutinin, β-galactosidase, thioreductase or an immunoglobulin domain or domains (for example, an Fc region). In preferred embodiments, a chimeric nucleic acid molecule of the invention encodes an FGF fusion polypeptide of the invention.

FGF nucleic acid molecules of the invention, whether genomic DNA, cDNA or otherwise, can be isolated from any source including, for example, cDNA or genomic libraries derived from a cell or cell line from an organism that has a FGF gene. In the case of cDNA libraries, such libraries are preferably derived from a cell or cell line that expresses an FGF gene. Methods for obtaining FGF genes are well known in the art, as described above (see, e.g., Sambrook et al., 1989, supra).

The DNA may be obtained by standard procedures known in the art from cloned DNA (for example, from a DNA “library”), and preferably is obtained from a cDNA library prepared from tissues with high level expression of the protein (e.g., from cells or from tissue. In one preferred embodiment, the DNA is obtained from a “subtraction” library to enrich the library for cDNAs of genes specifically expressed by a particular cell type or under certain conditions. Use of such a subtraction library may increase the likelihood of isolating cDNA for a particular gene, such as a particular FGF gene. In still other embodiments, a library may be prepared by chemical synthesis, by cDNA cloning, or by the cloning of genomic DNA or fragments thereof purified from the desired cell (See, for example, Sambrook et al., 1989, supra; Glover, D. M. ed., 1985, DNA Cloning: A Practical Approach, MRL Press, Ltd. Oxford, U.K. Vols. I and II).

In one embodiment, a cDNA library may be screened for an FGF nucleic acid by identifying cDNA inserts that encode a polypeptide which is homologous or substantially similar to an FGF polypeptide, such as the exemplary FGF2 polypeptide set forth in FIG. 1A (SEQ ID NO:1), the exemplary FGF1 polypeptide set forth in FIG. 16A (SEQ ID NO:5) or fragments thereof. Similarly, a cDNA library may be screened for an FGF nucleic acid by identifying cDNA inserts having a nucleic acid sequence that is homologous or substantially similar to an FGF nucleic acid sequence, such as the exemplary FGF2 nucleic acid sequence set forth in FIG. 1B (SEQ ID NO:2), the exemplary FGF1 nucleic acid sequence set forth in FIG. 16B (SEQ ID NO:6) or fragments thereof.

Clones derived from genomic DNA may contain regulatory and intron DNA regions in addition to coding regions. Clones derived from cDNA generally will not contain intron sequences. Whatever the source, the gene is preferably molecularly cloned into a suitable vector for propagation of the gene. Identification of the specific DNA fragment containing the desired FGF gene may be accomplished in a number of ways. For example, a portion of an FGF gene can be purified and labeled to prepare a labeled probe (Benton & Davis, Science 1977, 196:180; Grunstein & Hogness, Proc. Natl. Acad. Sci. U.S.A. 1975, 72:3961). Those DNA fragments with substantial homology to the probe (for example, an allelic variant from another individual, or a homologous FGF gene from the same or a different species of organism) will hybridize. In a specific embodiment, highest stringency hybridization conditions are used to identify a homologous FGF gene. However, lower (e.g., moderate) hybridization conditions may also be used.

Further selection can be carried out on the basis of the properties of the FGF gene product, e.g., if the gene encodes a protein product having the isoelectric, electrophoretic, amino acid composition, partial or complete amino acid sequence, antibody binding activity, or ligand binding profile of a FGF polypeptide. Thus, the presence of the gene may be detected by assays based on the physical, chemical, immunological, or functional properties of its expressed product.

Other DNA sequences which encode substantially the same amino acid sequence as a FGF gene may be used in the practice of the present invention. These include but are not limited to allelic variants, species variants, sequence conservative variants, and functional variants. In particular, the nucleic acid sequences of the invention include both “function-conservative variants” and “sequence-conservative variants”. Nucleic acid substitutions may be made for example, to alter the amino acid residue encoded by a particular codon, and thereby substitute an amino acid in a FGF polypeptide for one with a particularly preferable property. For example, a Cysteine amino acid residue may be introduced at a potential site for disulfide bridges with another Cysteine amino acid residue. Conversely, an amino acid residue, for example a Serine amino acid residue, may be substituted for a Cysteine amino acid residue in an FGF polypeptide. Such substitutions may be useful, for example, to facilitate solubilization of a recombinant FGF polypeptide.

The genes encoding FGF derivatives and analogs of the invention can be produced by various methods known in the art. The manipulations which result in their production can occur at the gene or protein level. For example, the cloned FGF gene sequence can be modified by any of numerous strategies known in the art (Sambrook et al., 1989, supra). The sequence can be cleaved at appropriate sites with restriction endonuclease(s), followed by further enzymatic modification if desired, isolated, and ligated in vitro. In the production of the gene encoding a derivative or analog of FGF, care should be taken to ensure that the modified gene remains within the same translational reading frame as the original FGF gene, uninterrupted by translational stop signals, in the gene region where the desired activity is encoded.

Additionally, the FGF-encoding nucleic acid sequence can be mutated in vitro or in vivo, to create and/or destroy translation, initiation, and/or termination sequences, or to create variations in coding regions and/or form new restriction endonuclease sites or destroy preexisting ones, to facilitate further in vitro modification. Modifications can also be made to introduce restriction sites and facilitate cloning the FGF gene into an expression vector. Any technique for mutagenesis known in the art can be used, including but not limited to, in vitro site-directed mutagenesis (Hutchinson, C., et al., J. Biol. Chem. 253:6551, 1978; Zoller and Smith, DNA 3:479-488, 1984; Oliphant et al., Gene 44:177, 1986; Hutchinson et al., Proc. Natl. Acad. Sci. U.S.A. 83:710, 1986), use of TAB” linkers (Pharmacia), etc. PCR techniques are preferred for site directed mutagenesis (see Higuchi, 1989, “Using PCR to Engineer DNA”, in PCR Technology: Principles and Applications for DNA Amplification, H. Erlich, ed., Stockton Press, Chapter 6, pp. 61-70).

The identified and isolated gene can then be inserted into an appropriate cloning vector. A large number of vector-host systems known in the art may be used. Possible vectors include, but are not limited to, plasmids or modified viruses, but the vector system must be compatible with the host cell used. Examples of vectors include, but are not limited to, E. coli, bacteriophages such as lambda derivatives, or plasmids such as pBR322 derivatives or pUC plasmid derivatives, e.g., pGEX vectors, pmal-c, pFLAG, pKK plasmids (Clonetech), pET plasmids (Novagen, Inc., Madison, Wis.), pRSET or pREP plasmids, pcDNA (Invitrogen, Carlsbad, Calif.), or pMAL plasmids (New England Biolabs, Beverly, Mass.), etc. The insertion into a cloning vector can, for example, be accomplished by ligating the DNA fragment into a cloning vector which has complementary cohesive termini. However, if the complementary restriction sites used to fragment the DNA are not present in the cloning vector, the ends of the DNA molecules may be enzymatically modified. Alternatively, any site desired may be produced by ligating nucleotide sequences (linkers) onto the DNA termini. These ligated linkers may comprise specific chemically synthesized oligonucleotides encoding restriction endonuclease recognition sequences.

Recombinant molecules can be introduced into host cells via transformation, transfection, infection, electroporation, etc., so that many copies of the gene sequence are generated. Preferably, the cloned gene is contained on a shuttle vector plasmid, which provides for expansion in a cloning cell, e.g., E. coli, and facile purification for subsequent insertion into an appropriate expression cell line, if such is desired. For example, a shuttle vector, which is a vector that can replicate in more than one type of organism, can be prepared for replication in both E. coli and Saccharomyces cerevisiae by linking sequences from an E. coli plasmid with sequences from the yeast 2m plasmid.

Expression of FGF polypeptides. A nucleotide sequence coding for an FGF polypeptide, for an antigenic fragment, derivative or analog of an FGF polypeptide, or for a functionally active derivative of an FGF polypeptide (including a chimeric protein) may be inserted into an appropriate expression vector, i.e., a vector which contains the necessary elements for the transcription and translation of the inserted protein-coding sequence. Thus, a nucleic acid encoding a FGF polypeptide of the invention can be operationally associated with a promoter in an expression vector of the invention. Both cDNA and genomic sequences can be cloned and expressed under control of such regulatory sequences. Such vectors can be used to express functional or functionally inactivated FGF polypeptides.

The necessary transcriptional and translational signals can be provided on a recombinant expression vector.

Potential host-vector systems include but are not limited to mammalian or other vertebrate cell systems transfected with expression plasmids or infected with virus (e.g., vaccinia virus, adenovirus, adeno-associated virus, herpes virus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used.

Expression of a FGF polypeptide may be controlled by any promoter/enhancer element known in the art, but these regulatory elements must be functional in the host selected for expression. Promoters which may be used to control FGF gene expression include, but are not limited to, cytomegalovirus (CMV) promoter (U.S. Pat. Nos. 5,385,839 and 5,168,062), the SV40 early promoter region (Benoist and Chambon, Nature 1981, 290:304-310), the promoter contained in the 3′ long terminal repeat of Rous sarcoma virus (Yamamoto, et al., Cell 1980, 22:787-797), the herpes thymidine kinase promoter (Wagner et al., Proc. Natl. Acad. Sci. U.S.A. 1981, 78:1441-1445), the regulatory sequences of the metallothionein gene (Brinster et al., Nature 1982, 296:39-42); prokaryotic expression vectors such as the b-lactamase promoter (Villa-Komaroff, et al., Proc. Natl. Acad. Sci. U.S.A. 1978, 75:3727-3731), or the tac promoter (DeBoer, et al., Proc. Natl. Acad. Sci. U.S.A. 1983, 80:21-25, 1983); see also “Useful proteins from recombinant bacteria” in Scientific American 1980, 242:74-94. Still other useful promoter elements which may be used include promoter elements from yeast or other fungi such as the Gal 4 promoter, the ADC (alcohol dehydrogenase) promoter, PGK (phosphoglycerol kinase) promoter, alkaline phosphatase promoter; and transcriptional control regions that exhibit hematopoietic tissue specificity, in particular: beta-globin gene control region which is active in myeloid cells (Mogram et al., Nature 1985, 315:338-340; Kollias et al., Cell 1986, 46:89-94), hematopoietic stem cell differentiation factor promoters, erythropoietin receptor promoter (Maouche et al., Blood 1991, 15:2557), etc.

Indeed, any type of plasmid, cosmid, YAC or viral vector may be used to prepare a recombinant nucleic acid construct which can be introduced to a cell, or to tissue, where expression of an FGF gene product is desired. Alternatively, wherein expression of a recombinant FGF gene product in a particular type of cell or tissue is desired, viral vectors that selectively infect the desired cell type or tissue type can be used.

In another embodiment, the invention provides methods for expressing FGF polypeptides by using a non-endogenous promoter to control expression of an endogenous FGF gene within a cell. An endogenous FGF gene within a cell is an FGF gene of the present invention which is ordinarily (i.e., naturally) found in the genome of that cell. A non-endogenous promoter, however, is a promoter or other nucleotide sequence that may be used to control expression of a gene but is not ordinarily or naturally associated with the endogenous FGF gene. As an example, methods of homologous recombination may be employed (preferably using non-protein encoding FGF nucleic acid sequences of the invention) to insert an amplifiable gene or other regulatory sequence in the proximity of an endogenous FGF gene. The inserted sequence may then be used, e.g., to provide for higher levels of FGF gene expression than normally occurs in that cell, or to overcome one or more mutations in the endogenous FGF regulatory sequences which prevent normal levels of FGF gene expression. Such methods of homologous recombination are well known in the art. See, for example, International Patent Publication No. WO 91/06666, published May 16, 1991 by Skoultchi; International Patent Publication No. WO 91/099555, published Jul. 11, 1991 by Chappel; and International Patent Publication No. WO 90/14092, published Nov. 29, 1990 by Kucherlapati and Campbell.

Soluble forms of the protein can be obtained by collecting culture fluid, or solubilizing inclusion bodies, e.g., by treatment with detergent, and if desired sonication or other mechanical processes, as described above. The solubilized or soluble protein can be isolated using various techniques, such as polyacrylamide gel electrophoresis (PAGE), isoelectric focusing, 2-dimensional gel electrophoresis, chromatography (e.g., ion exchange, affinity, immunoaffinity, and sizing column chromatography), centrifugation, differential solubility, immunoprecipitation, or by any other standard technique for the purification of proteins.

A wide variety of host/expression vector combinations may be employed in expressing the DNA sequences of this invention. Useful expression vectors, for example, may consist of segments of chromosomal, non-chromosomal and synthetic DNA sequences. Suitable vectors include derivatives of SV40 and known bacterial plasmids, e.g., E. coli plasmids col E1, pCR1, pBR322, pMal-C2, pET, pGEX (Smith et al., Gene 1988, 67:31-40), pCR2.1 and pcDNA 3.1+(Invitrogen, Carlsbad, Calif.), pMB9 and their derivatives, plasmids such as RP4; phage DNAs, e.g., the numerous derivatives of phage 1, e.g., NM989, and other phage DNA, e.g., M13 and filamentous single stranded phage DNA; yeast plasmids such as the 2m plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

Preferred vectors are viral vectors, such as lentiviruses, retroviruses, herpes viruses, adenoviruses, adeno-associated viruses, vaccinia virus, baculovirus, and other recombinant viruses with desirable cellular tropism. Thus, a gene encoding a functional or mutant FGF polypeptide or a domain fragment thereof can be introduced in vivo, ex vivo, or in vitro using a viral vector or through direct introduction of DNA. Expression in targeted tissues can be effected by targeting the transgenic vector to specific cells, such as with a viral vector or a receptor ligand, or by using a tissue-specific promoter, or both. Targeted gene delivery is described in International Patent Publication WO 95/28494, published October 1995.

Viral vectors commonly used for in vivo or ex vivo targeting and therapy procedures are DNA-based vectors and retroviral vectors. Methods for constructing and using viral vectors are known in the art (see, e.g., Miller and Rosman, BioTechniques 1992, 7:980-990). Preferably, the viral vectors are replication defective, that is, they are unable to replicate autonomously in the target cell. In general, the genome of the replication defective viral vectors which are used within the scope of the present invention lack at least one region which is necessary for the replication of the virus in the infected cell. These regions can either be eliminated (in whole or in part), be rendered non-functional by any technique known to a person skilled in the art. These techniques include the total removal, substitution (by other sequences, in particular by the inserted nucleic acid), partial deletion or addition of one or more bases to an essential (for replication) region. Such techniques may be performed in vitro (on the isolated DNA) or in situ, using the techniques of genetic manipulation or by treatment with mutagenic agents. Preferably, the replication defective virus retains the sequences of its genome which are necessary for encapsidating the viral particles.

DNA viral vectors include an attenuated or defective DNA virus, such as but not limited to herpes simplex virus (HSV), papillomavirus, Epstein Barr virus (EBV), adenovirus, adeno-associated virus (AAV), and the like. Defective viruses, which entirely or almost entirely lack viral genes, are preferred. Defective virus is not infective after introduction into a cell. Use of defective viral vectors allows for administration to cells in a specific, localized area, without concern that the vector can infect other cells. Thus, a specific tissue can be specifically targeted. Examples of particular vectors include, but are not limited to, a defective herpes virus 1 (HSV1) vector (Kaplitt et al., Molec. Cell. Neurosci. 1991, 2:320-330), defective herpes virus vector lacking a glyco-protein L gene (Patent Publication RD 371005 A), or other defective herpes virus vectors (International Patent Publication No. WO 94/21807, published Sep. 29, 1994; International Patent Publication No. WO 92/05263, published Apr. 2, 1994); an attenuated adenovirus vector, such as the vector described by Stratford-Perricaudet et al. (J. Clin. Invest. 1992, 90:626-630; see also La Salle et al., Science 1993, 259:988-990); and a defective adeno-associated virus vector (Samulski et al., J. Virol. 1987, 61:3096-3101; Samulski et al., J. Virol. 1989, 63:3822-3828; Lebkowski et al., Mol. Cell. Biol. 1988, 8:3988-3996).

Various companies produce viral vectors commercially, including but by no means limited to Avigen, Inc. (Alameda, Calif.; AAV vectors), Cell Genesys (Foster City, Calif.; retroviral, adenoviral, AAV vectors, and lentiviral vectors), Clontech (retroviral and baculoviral vectors), Genovo, Inc. (Sharon Hill, Pa.; adenoviral and AAV vectors), Genvec (adenoviral vectors), IntroGene (Leiden, Netherlands; adenoviral vectors), Molecular Medicine (retroviral, adenoviral, AAV, and herpes viral vectors), Norgen (adenoviral vectors), Oxford BioMedica (Oxford, United Kingdom; lentiviral vectors), Transgene (Strasbourg, France; adenoviral, vaccinia, retroviral, and lentiviral vectors) and Invitrogen (Carlbad, Calif.).

In another embodiment, the vector can be introduced in vivo by lipofection, as naked DNA, or with other transfection facilitating agents (peptides, polymers, etc.). Synthetic cationic lipids can be used to prepare liposomes for in vivo transfection of a gene encoding a marker (Felgner et al., Proc. Natl. Acad. Sci. U.S.A. 1987, 84:7413-7417; Felgner and Ringold, Science 1989, 337:387-388; Mackey et al., Proc. Natl. Acad. Sci. U.S.A. 1988, 85:8027-8031; Ulmer et al., Science 1993, 259:1745-1748). Useful lipid compounds and compositions for transfer of nucleic acids are described in International Patent Publications WO 95/18863 and WO 96/17823, and in U.S. Pat. No. 5,459,127. Lipids may be chemically coupled to other molecules for the purpose of targeting (see, Mackey et al., Proc. Natl. Acad. Sci. U.S.A. 1988, 85:8027-8031). Targeted peptides, e.g., hormones or neurotransmitters, and proteins such as antibodies, or non-peptide molecules could be coupled to liposomes chemically. Other molecules are also useful for facilitating transfection of a nucleic acid in vivo, such as a cationic oligopeptide (e.g., International Patent Publication WO 95/21931), peptides derived from DNA binding proteins (e.g., International Patent Publication WO 96/25508), or a cationic polymer (e.g., International Patent Publication WO 95/21931).

It is also possible to introduce the vector in vivo as a naked DNA plasmid. Naked DNA vectors for gene therapy can be introduced into the desired host cells by methods known in the art, e.g., electroporation, microinjection, cell fusion, DEAE dextran, calcium phosphate precipitation, use of a gene gun, or use of a DNA vector transporter (see, e.g., Wu et al., J. Biol. Chem. 1992, 267:963-967; Wu and Wu, J. Biol. Chem. 1988, 263:14621-14624; Hartmut et al., Canadian Patent Application No. 2,012,311, filed Mar. 15, 1990; Williams et al., Proc. Natl. Acad. Sci. U.S.A. 1991, 88:2726-2730). Receptor-mediated DNA delivery approaches can also be used (Curiel et al., Hum. Gene Ther. 1992, 3:147-154; Wu and Wu, J. Biol. Chem. 1987, 262:4429-4432). U.S. Pat. Nos. 5,580,859 and 5,589,466 disclose delivery of exogenous DNA sequences, free of transfection facilitating agents, in a mammal. Recently, a relatively low voltage, high efficiency in vivo DNA transfer technique, termed electrotransfer, has been described (Mir et al., C.P. Acad. Sci. 1998, 321:893; WO 99/01157; WO 99/01158; WO 99/01175).

Preferably, for in vivo administration, an appropriate immunosuppressive treatment is employed in conjunction with the viral vector, e.g., adenovirus vector, to avoid immuno-deactivation of the viral vector and transfected cells. For example, immunosuppressive cytokines, such as interleukin-12 (IL-12), interferon-g (IFN-γ), or anti-CD4 antibody, can be administered to block humoral or cellular immune responses to the viral vectors (see, e.g., Wilson, Nat. Med. 1995, 1:887-889). In that regard, it is advantageous to employ a viral vector that is engineered to express a minimal number of antigens.

### FGF Receptors

FGF receptor polypeptides. The present invention relates, not only to FGF ligand polypeptides, described supra, but also to receptor polypeptides that specifically bind to an FGF polypeptide. Such receptor polypeptides are generally referred to as FGF receptor polypeptides or FGFR polyeptides.

In preferred embodiments, an FGFR polypeptide of the invention is characterized by its biological activity or activities; i.e., an FGFR polypeptide of the invention is able to specifically bind to an FGF polypeptide. Preferably, the FGFR polypeptide also has a tyrosine kinase activity that may be activated upon binding of the receptor to an FGF ligand and/or upon dimerization of the FGF receptor (i.e., by the binding of a first FGFR polypeptide to a second, preferably identical, FGFR polypeptide). Activation of an FGFR polypeptide may also stimulate one or more biological activities that are associated with FGF signaling. For example, activation of an FGFR polypeptide in cells (e.g., by binding an FGF ligand and/or receptor dimerization) may stimulate activities such as cell mitogenesis or angiogenesis.

FGFR polypeptides, like their ligands, are known in the art (see, in particular, the references cited, supra). In particular, at least four types of FGFR polypeptide, known individually as FGFR1—FGFR4, are believed to exist (see, e.g., Jaye et al., Biochimica et Biophysica Acta 1992, 1135:185-199). Each of these FGFR polypeptides comprises a cytoplasmic domain that typically exhibits a tyrosine kinase activity, a transmembrane helix domain, and an extracellular domain. The extracellular domain normally recognizes and specifically binds to an FGF ligand, and may itself comprise at least three distinct immunoglobulin (Ig)-like domains referred to as D1-D3. Binding specificity for the FGF ligand typically resides in, and is therefore incurred by, the D2 and D3 domains and by the short linker polypeptide sequence between those two domains. See, Plotnikov et al., Cell 1999, 98:641-650; Plotnikov et al., Cell 2000, 101:413-424; and Stauber et al., Proc. Natl. Acad. Sci. U.S.A. 2000, 97:49-54 for a more detailed discussion.

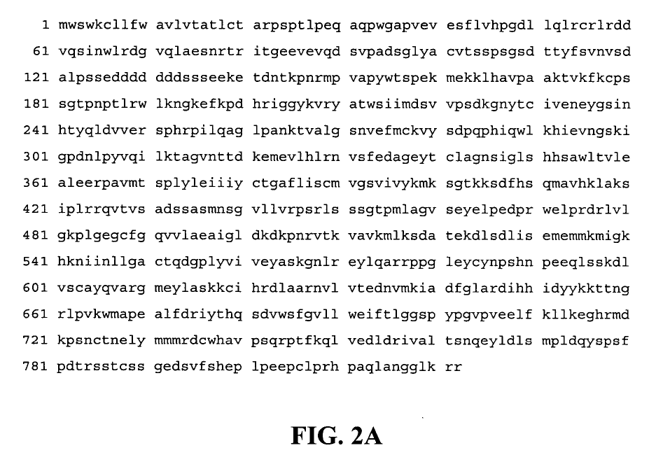

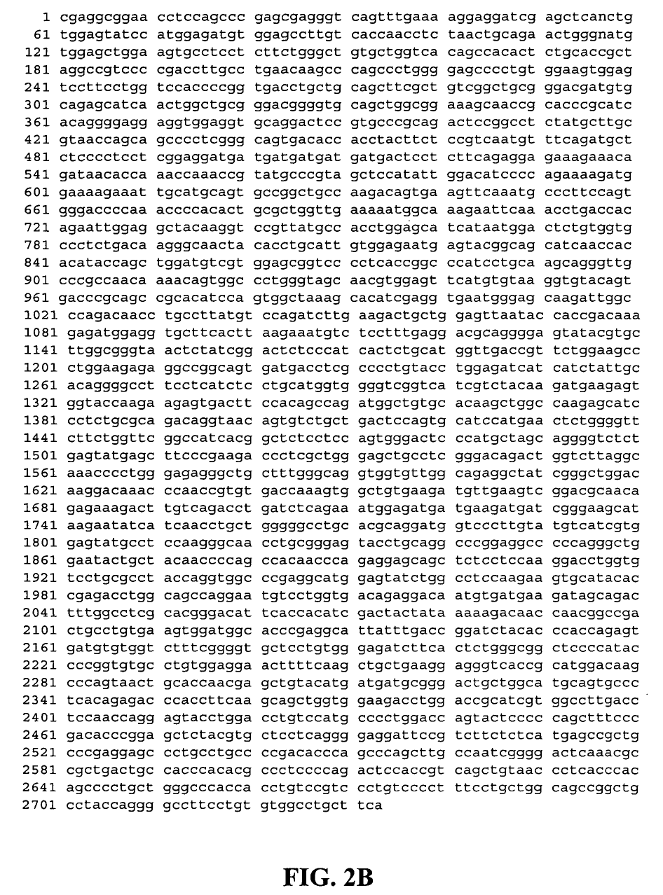

The amino acid sequence for an exemplary FGFR polypeptide, known as FGFR1, is shown here in FIG. 2A (SEQ ID NO:3). The FGFR1 amino acid sequence is also available from GenBank and has the Accession No. P11362 (GI:120046). In this exemplary FGFR polypeptide, the D1 domain corresponds to amino acid residues 30-119. The D2 domain corresponds to amino acid residues 149-247, whereas the D3 domain corresponds to amino acid residues 252-359. The amino acid residues connecting the D1 and D2 domains (i.e., residues 120-148) are referred to here as the D1-D2 “linker region” or the D1-D2 “linker”. Similarly, amino acid residues connecting the D2 and D3 domains (i.e., residues 248-251) are referred to here as the D2-D3 “linker region” or the D2-D3 “linker”. It is understood that, in preferred embodiments, the amino acid residue numbers used to delineate these separate domains are approximate.

As noted above, numerous variants (including homologs and orthologs from the same and different species of organisms) are known in the art and/or may be readily identified. Such variants, including any of the FGFR polypeptides known as FGFR1, FGFR2, FGFR3 or FGFR4, are also considered part of the present invention and may be used in the compositions and methods described herein. Such variant sequences may be identified using any of the methods described, supra, to identify variants (including orthologs and homologs) of an FGF polypeptide.

Thus, for example, the FGFR polypeptides of the invention also include ones encoded by nucleic acid molecules that hybridize to the complement of a nucleic acid molecule encoding another FGFR polypeptide (e.g., in a Southern hybridization experiment under defined conditions). For example, in particular embodiments, an FGF polypeptide may comprise an amino acid sequence encoded by a nucleic acid molecule that hybridizes to the complement of an FGFR1 nucleic acid sequence, such as the coding sequence set forth in FIG. 2B (SEQ ID NO:4), under highly stringent conditions that comprise 50% formamide in 5× or 6×SSC. In other embodiments, the FGF polypeptide may comprise an amino acid sequence encoded by nucleic acid molecules that hybridize to a complement of an FGFR nucleic acid sequence (e.g., the coding sequence in FIG. 2B and SEQ ID NO:4) under moderately stringent hybridization conditions (for example, 40% formamide with 5× or 6×SSC), or under low stringency conditions (for example in 5×SSC, 0.1% SDS, 0.25% milk, no formamide, 5×SSC, or 0.5% SDS).

In still other embodiments, FGFR polypeptides can also be identified by isolating homologous or variant FGFR gene, e.g., by PCR using degenerate oligonucleotide primes designed on the basis of a given FGFR polypeptide sequence as described below.

FGFR polypeptides of the invention also include polypeptides that comprise one or more partial or fragment FGFR amino acid sequences; i.e., a portion or fragment of a full length FGFR amino acid sequence such as the full length FGFR1 sequence shown in FIG. 2A (SEQ ID NO:3). Such partial FGFR polypeptides may comprise, for example, an amino acid sequence of one or more epitopes or domains of a full length FGFR polypeptide. In one preferred embodiment, for example, a partial FGFR polypeptide comprises an amino acid sequence corresponding to at least one domain which may be, e.g., an intracellular domain, a transmembrane domain, or an extracellular domain such as a D1, D2 or D3 domain. A partial FGFR polypeptide may also comprise an amino acid sequence corresponding to a combination of two or more domains from a full length FGFR polypeptide. For instance, the examples, infra, described the construction of an exemplary fusion polypeptide that comprises the D2 and D3 domain of the FGFR1 polypeptide sequence set forth in FIG. 2A (SEQ ID NO:3).

Partial FGFR polypeptides of the invention also include ones that comprise an amino acid sequence of one or more epitopes of a full length FGFR polypeptide. Preferably, such polypeptides contain an amino acid sequence corresponding to at least 5, at least 10, at least 15, at least 20, at least 25, or at least 50 amino acid residues of a full length FGFR polypeptide sequence (e.g., of the full length FGFR1 amino acid sequence set forth in FIG. 2A and in SEQ ID NO:3).

Truncated forms of an FGFR polypeptide can also be provided. Such truncated forms may include an FGFR polypeptide with a specific deletion of amino acid residues. For instance, in certain embodiments amino acid residue corresponding to one or more domains of a full length FGFR polypeptide (e.g., one or more of the particular domains described, above) may be deleted from the amino acid sequence of an FGFR polypeptide.

The FGFR polypeptides of this invention include, in addition to naturally occurring homologs and orthologs of FGFR polypeptides such as FGFR1 (SEQ ID NO:3), but also include analogs and derivatives of an FGFR polypeptide. Such analogs and derivatives may be ones that are naturally occurring (such as allelic variants), or may be man made (such as fusion proteins). However, analogs and derivatives of an FGFR polypeptide will have the same or homologous characteristics of FGFR polypeptides set forth above.

An FGFR chimeric or fusion polypeptide may also be prepared in which the FGFR portion of the fusion polypeptide has one or more characteristics of the FGFR polypeptide. Such fusion polypeptides therefore represent alternative embodiments of the FGFR polypeptides of this invention. Exemplary FGFR fusion polypeptides include ones which comprise a full length, derivative or truncated FGFR amino acid sequence, as well as fusions which comprise a fragment of an FGFR polypeptide sequence (e.g., a fragment corresponding to an epitope or to one or more domains). Such fusion polypeptides may also comprise the amino acid sequence of a second, different polypeptides; including the amino acid sequence for any of the poylpeptides described, supra, for fusion proteins of an FGF ligand.

FGFR analogs or variants can also be made by altering encoding nucleic acid molecules, including any of the alterations described, supra, for FGF ligand polypeptides (e.g., by substitutions, additions or deletions). Preferably, such altered nucleic acid molecules encode functionally similar molecules (i.e., molecules that perform one or more functions of an FGFR polypeptide and/or have one or more FGFR bioactivities). Thus, in a specific embodiment, an analog or variant of an FGFR polypeptide is a function-conservative analog or variant.

As with FGF ligand polypeptides, amino acid residues (other than ones that are specifically identified herein as being conserved) may differ among variants of a protein or polypeptide. Accordingly, the percentage of protein or amino acid sequence similarity between any two FGFR polypeptides may vary. The skilled artisan will recognize that the percentage of protein or amino acid sequence similarity between any two FGFR polypeptides of similar function may vary in ways that are similar to those sequence variations described, supra, for FGF ligand polypeptides and nucleic acids.

In still other embodiments, an analog of an FGFR polypeptide may be an allelic variant or mutant FGFR polypeptide. The FGFR polypeptides of the invention also include derivative FGFR polypeptides which may be modified, e.g., according to any of the specific modifications described, supra, for FGF polypeptides.

While the above, exemplary variants and analogs of FGFR polypeptides are described primarily in terms of the exemplary FGFR polypeptide, FGFR1, set forth in FIG. 2A (SEQ ID NO:3), it is understood that variant FGFR polypeptides of the invention include other FGFR polypeptides (e.g., naturally occurring homologs and orthologs described supra) having equivalent amino acid substitutions, deletions or insertions.

FGF receptor nucleic acids. In general, an FGFR nucleic acid molecule of the present invention comprises a nucleic acid sequence that encodes an FGFR polypeptide (as defined, above, in this Subsection) or the complement of an FGFR polypeptide encoding sequence. The invention also provides fragments of FGFR encoding sequences and their complements, and such sequences are also considered part of the FGFR nucleic acid molecules of this invention. Thus, in one exemplary embodiment, an FGFR nucleic acid molecule of this invention may encode the exemplary FGFR1 polypeptide sequence set forth in FIG. 2A (SEQ ID NO:3), such as the particular FGFR1 nucleic acid sequence that is depicted in FIG. 2B (SEQ ID NO:4).

In still other embodiment, the FGFR nucleic acid molecules of this invention comprise nucleic acid sequences that encode one or more domains of an FGFR polypeptide; for example, an intracellular domain, a transmembrane domain, or an extracellular domain or portion thereof (e.g., a D1, D2 or D3 domain).

The FGFR nucleic acid molecules of the invention also include nucleic acids which comprise a sequence encoding one or more fragments of an FGFR polypeptide. Such fragments include, for example, polynucleotides that encode an epitope of an FGFR polypeptide; e.g., nucleic acids that encode a sequence of at least 5, and more preferably at least 10, 15, 20, 25 or 50 amino acid residues of an FGFR polypeptide sequence (for example, the exemplary FGFR1 polypeptide sequence set forth in FIG. 2B and in SEQ ID NO:4).

As explained above, numerous variant FGFR polypeptides are known in the art and/or may be readily identified by those skilled in the art, including homologous and orthologous polypeptides from the same and different species of organism. The FGFR nucleic acid molecules of the invention therefore include nucleic acid molecule comprising coding sequences for variant FGFR polypeptides (including allelic variants, analogs and homologous from the same or different species), as well as nucleic acid molecule comprising coding sequences for modified FGFR polypeptides (e.g., having amino acid substitutions, deletions or truncations). In preferred embodiments, such nucleic acid molecules have at least 50%, preferably at least 75% and more preferably at least 90% sequence identity to another FGFR coding sequence, such as the exemplary FGF2 coding sequence set forth in FIG. 2B (SEQ ID NO:4).

In addition, the FGFR nucleic acid molecules of the invention include nucleic acid molecules that hybridize to another FGFR nucleic acid molecule, e.g., in a Southern blot assay under defined conditions. For example, in specific embodiments an FGF nucleic acid molecule of the invention comprises a nucleotide sequence which hybridizes to a complement of the exemplary FGFR1 coding sequence set forth in FIG. 2B (SEQ ID NO:4) under highly stringent or moderately stringent hybridization conditions that are defined, supra, for FGF nucleic acids. Alternatively, an FGFR nucleic acid molecule may hybridize, under the same defined hybridization conditions, to the complement of a fragment of a nucleotide sequence encoding a full length FGFR polypeptide.

In other embodiments, FGFR nucleic acid molecules of the invention comprise fragments of a full length FGFR nucleic acid sequence. Such nucleic acid fragments comprise a nucleotide sequence that corresponds to a sequence of at least 10 nucleotides, preferably at least 15 nucleotides and more preferably at least 20 nucleotides of a full length coding FGFR nucleotide sequence. In specific embodiments, the fragments correspond to a portion (e.g., of at least 10, 15, or 20 nucleotides) of the exemplary FGFR1 coding sequence shown in FIG. 2B (SEQ ID NO:4). In other embodiments, an FGFR nucleic acid fragment may comprise sequences of at least 10, preferably at least 15, and more preferably at least 20 nucleotides that are complementary and/or hybridize to a full length FGFR coding sequence (e.g., the FGFR1 coding sequence set forth in FIG. 2B and in SEQ ID NO:4) or to a fragment thereof. Suitable hybridization conditions for such oligonucleotides are described, supra, for FGF nucleic acids.

Nucleic acid molecules comprising such fragments are useful, for example, as oligonucleotide probes and primers (e.g., PCR primers) to detect and amplify other nucleic acid molecules encoding an FGFR polypeptide, including genes that encode variant FGFR polypeptides (including genes that encode homologous or orthologous FGFR polypeptides from the same or different species of organism). Oligonucleotide fragments of the invention may also be used, e.g., as antisense nucleic acids, triple helix forming oligonucleotides or as ribozymes (e.g., to modulate levels of FGFR gene expression or transcription in cells).

The nucleic acid molecules of the invention also include “chimeric” FGFR nucleic acid molecules. Such chimeric nucleic acid molecules are polynucleotides which comprise at least one FGFR nucleic acid sequence (which may be any of the full length or partial FGFR nucleic acid sequences described above), and also at least one non-FGFR nucleic acid sequence. For example, the non-FGFR nucleic acid sequence may be any of the non-FGF nucleic acid sequences described, supra. In preferred embodiments, a chimeric FGFR nucleic acid molecule of the invention encodes an FGFR fusion polypeptide of the invention.

It is understood that FGFR nucleic acid molecules of the present invention may be obtained and/or isolated using standard techniques that are known in the art and described, supra, for obtaining FGF nucleic acids. Similarly, FGFR polyeptides may be readily expressed, e.g., by expressing FGFR nucleic acids in host cells using any of the art recognized techniques that are described above for expressing FGF polypeptides.

### Agonists and Antagonists

The present invention also provides compounds that modulate FGFR activity and FGF-signaling. Such compounds are therefore useful, e.g., for modulating biological activities that are associated with FGF-signaling and/or as therapeutic agents for treating disorders associated with FGF-signaling. For example, the compounds of this invention may be used, e.g., to modulate mitogenesis, angiogenesis or differentiation of cells. Such compounds are also useful, e.g., as therpeutic agents to modulate tumor growth or to treat a disorder of cell proliferation (referred to herein as “cell proliferation disorders”), for example cancer.

Compounds that modulate FGF-signaling or an activity associated therewith may be readily identified using screening methods of the present invention. For example, the accompanying appendix provides structure coordinates, discussed in the Examples infra, for a dimerized ternary complex of an FGF ligand, an FGF receptor and sucrose octasulfate (SOS). Interactions (e.g., hydrogen bonding interactions) between the SOS molecule and the FGF ligand and receptor molecule(s) are also disclosed that stabilize formation of the ternary complex and, moreover, stabilize FGF receptor dimerization. Using routine, computer modeling algorithms and other techniques that are well known in the art, a user may identify other compounds that are expected to an FGF ligand and/or its receptor in a way that is similar to binding of SOS. More specifically, using the crystal structure provided here, those skilled in the art can identify compounds that bind to an FGF receptor and/or ligand, and form stabilizing interactions with the ligand/receptor complex that are similar to the stabilizing interactions described here for SOS.

In exemplary embodiments, compounds identified by the screening methods of this invention may form a ternary complex with an FGF ligand and its receptor while, at the same time, inhibiting FGF receptor dimerization. More specifically, the compounds may be ones which have (or are expected to have) stabilizing interactions between an FGF ligand and receptor in a ternary complex that are similar to the stabilizing interactions described infra, for SOS. At the same time, however, these compounds may disrupt or inhibit stabilizing interactions between a first and second ternary complex (e.g., by eliminating key hydrogen bonding interactions) so that dimerization of the FGF receptor is inhibited. Such compounds can be expected to compete with heparin for binding to the FGF ligand and its receptor, and inhibit FGFR dimerization. Accordingly, the compounds can also be expected to inhibit FGFR activity and FGF-signaling, as well as biological activities (e.g., mitogenesis, angiogenesis, etc.) that are associated with FGF-signaling and FGFR activity. Still other compounds, such as suramin, described infra, may stabilize interactions between an FGF-ligand and its receptor, similar to SOS, while at the same time inhibiting FGF signaling. Such compounds are therefore referred to here as “antagonists” or as “heparin antagonists” since they suppress the action of heparin in FGF-signaling.

In other exemplary embodiments, compounds identified by screening methods of this invention may actually have (or may be expected to have) improved binding or stabilizing interactions with an FGF ligand and/or receptor(s). For example, compounds identified by these screening methods may form (or be expected to form) stronger and/or more specific hydrogen bonding interactions with an FGF ligand or with an FGF receptor or recptors, and may actually form complexes with an increased binding affinity relative, e.g., to heparin. Such compound may also promote dimerization of an FGF receptor and thereby increasing FGFR dimerization. These compounds can be expected to increase FGFR activity and FGF-signaling, as well as biological activities that are associated with FGF-signaling and FGFR activity. Such compounds are therefore referred to here as “agonists” or “heparin agonists” since they enhance or improve upon the action of heparin in FGF-signaling.

Examples of heparin agonists and antagonists include derivatives of SOS. SOS derivatives may be determined using a rational drug design approach that utilizes the information derived from the FGF-FGFR-SOS complex crystal structure described in the Examples, infra. Examples of antagonists include suramin and SOS derivatives with one or more sulfate groups substituted with benzyl or trityl or other bulky hydroxylprotecting groups. Bulky groups such as these are predicted to provide a steric effect, which hampers recruitment of a second FGFR from another FGF-FGFR complex.

SOS derivatives, which incorporate benzyl and trityl substitutions or other bulky group substitutions may be synthesized using regioselective sucrose functionalization procedures known to those skilled in the art (see, for example, Jenner & Khan, J.C.S. Chem. Comm. 1980, 50-51; Vlahov, J. Carbohydr. Chem. 1997, 16:1-10; Polat, J. Carbohydr. Chem. 1997, 16:1319-1325; and Bazin, Carbohydr. Res. 1998, 309:189-205), followed by persulfonation. Other types of hydroxylprotecting groups, such as bulky acyl groups, including but not limited to benzoyl, pivaloyl, fatty acyl groups, or bulky silyl groups such as t-butylphenylsilyl (TBDPS) or t-butylmethylsilyl (TBDMS), or bulky ketals or acetals such as isopropylidene or benzylidene, might also be used in place of the bulky benzyl and trityl ether groups.

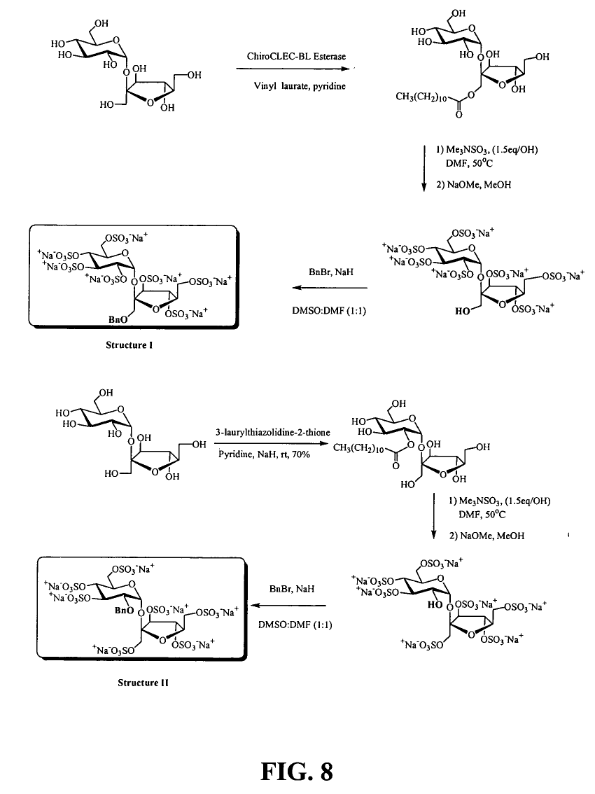

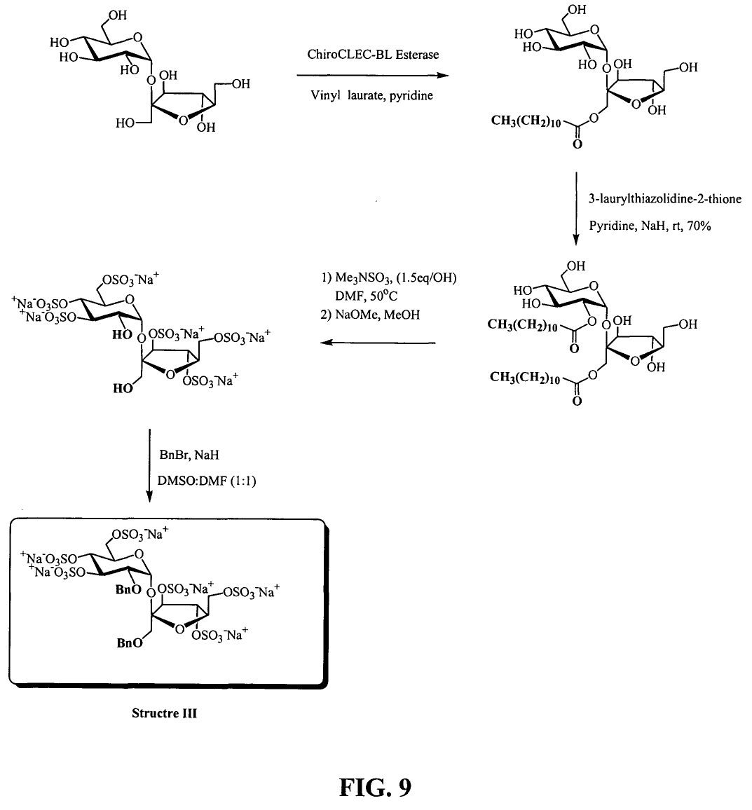

Preferred SOS derivatives include 2-O-Bn sucrose heptasulfate (Structure I), 1′-O-Bn sucrose heptasulfate (Structure II), 1′,2-di-O-Bn sucrose hexasulfate (Structure III). The exemplary synthesis of Structures I and II is illustrated in FIG. 8. The exemplary synthesis of Structure m is illustrated in FIG. 9. Specifically, structures I and II may be formed by the selective benzylation of sucrose in the 1′- or 2-positions, followed by separation and persulfonation. Structure III may be formed using a regioselective 1′,2-silylation (Jenner & Khan, supra) followed by peracetylation and separation. The 1′,2-silyl derivative formed is desialated, the free hydroxy groups are benzylated, and the compound formed is deacetylated and persulfonated.

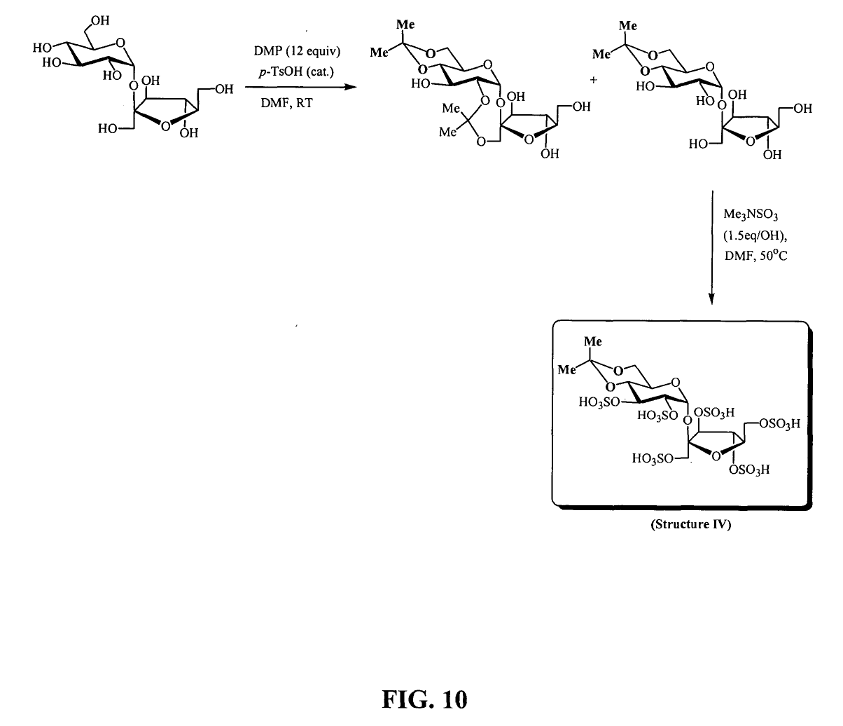

Still other examplary SOS derivatives include 6-O-hexadecanoyl sucrose heptasulfate (Structure V) and 2-)-dodecanoyl, 6′-O-hexadecanoyl sucrose hexasulfate (Structure VI), both of which are illustrated in FIG. 10.

Compounds identified by molecular modeling and/or the screening methods described here may be further investigated to better characterize their ability to form ternary complexes with FGF ligands and receptor, as well as for their ability to modulate FGFR dimerization and FGF-signaling. For example, a test compound may be contacted, in a reaction mixture, to an FGF ligand, and to an FGF receptor in either the presence or, alternatively, in the absence of co-factors such as heparin. The reaction mixture can then be assayed to determine whether a ternary complex has formed using techniques, such as size exclusion chromatography (see the Examples, infra), that are well known in the art. In preferred embodiments, such assays may also determine whether such ternary complexes have dimerized to indicate whether FGFR dimerization has been enhanced or inhibited by the test compound.

In vivo or cell culture assays may also be used to determine whether a test compound functions as a heparin agonist or antagonist to modulate FGFR activity or FGF-signaling in cells. For instance, the Examples, infra, describe cell culture assays that may be used to measure a test compound's ability to modulate an activity, such as mitogenesis, that is associated with FGF-signaling. Such assays generally comprise contacting a test compound to a cell that expresses an FGF receptor. The test compound should be contacted to the cell in the presence of an FGF ligand and, optionally, in the presence of a co-factor such as heparin or HSPG that activates FGFR. The cell culture may then be assayed or examined to determine whether a response associated with FGF-signaling has been activated. For instance, the Examples infra provide an assay that test the ability of a test compound to modulate cell growth (i.e., mitogenesis) stimulated by FGF-signaling.

Pharmaceutical Preparations. In preferred embodiments, compounds that are agonists or antagonists of FGFR activity and/or of FGF-signaling may be administered (e.g., in vitro or ex vivo to cell cultures, or in vivo to an organism) at therapeutically effective doses to treat a disease or disorder associated with FGF-signaling. Such compounds may be used, for example, to modulate activities such a mitogenesis and angiogenesis, or to modulate (preferably decrease) tumor growth. Exemplary diseases that may be treated using such methods include cell proliferative disorders such as cancer. Accordingly, the invention also provides pharmaceutical preparations for use, e.g., as therapeutic compounds for the treatment of disorders and other conditions that are associated with FGF-signaling and/or FGFR activity.

The terms “therapeutically effective dose” and “effective amount” refer to the amount of the compound that is sufficient to result in a therapeutic response. In embodiments where a compound (e.g., a drug or toxin) is administered in a complex (e.g., with an FGF or FGFR specific antibody), the terms “therapeutically effective dose” and “effective amount” may refer to the amount of the complex that is sufficient to result in a therapeutic response. A therapeutic response may be any response that a user (e.g., a clinician) will recognize as an effective response to the therapy. Thus, a therapeutic response will generally be an amelioration of one or more symptoms of a disease or disorder. In preferred embodiments, where the pharmaceutical preparations are used to treat a cancer, a therapeutic response may be a reduction in the number of cancer cells observed, e.g., in biopsies from a patient during treatment. Alternatively, an effective therapeutic response may be a reduction or shrinkage in the size of one or more tumors.

Toxicity and therapeutic efficacy of compounds can be determined by standard pharmaceutical procedures, for example in cell culture assays or using experimental animals to determine the LD₅₀ and the ED₅₀. The parameters LD₅₀ and ED₅₀ are well known in the art, and refer to the doses of a compound that are lethal to 50% of a population and therapeutically effective in 50% of a population, respectively. The dose ratio between toxic and therapeutic effects is referred to as the therapeutic index and may be expressed as the ratio: LD₅₀/ED₅₀. Compounds that exhibit large therapeutic indices are preferred. While compounds that exhibit toxic side effects may be used. However, in such instances it is particularly preferable to use delivery systems that specifically target such compounds to the site of affected tissue so as to minimize potential damage to other cells, tissues or organs and to reduce side effects.

Data obtained from cell culture assay or animal studies may be used to formulate a range of dosages for use in humans. The dosage of compounds used in therapeutic methods of the present invention preferably lie within a range of circulating concentrations that includes the ED₅₀ concentration but with little or no toxicity (e.g., below the LD₅₀ concentration). The particular dosage used in any application may vary within this range, depending upon factors such as the particular dosage form employed, the route of administration utilized, the conditions of the individual (e.g., patient), and so forth.

A therapeutically effective dose may be initially estimated from cell culture assays and formulated in animal models to achieve a circulating concentration range that includes the IC₅₀. The IC₅₀ concentration of a compound is the concentration that achieves a half-maximal inhibition of FGF signaling activity (e.g., as determined from the cell culture assays) or, where a compound is administered to treat a particular disorder, a half-maximal inhibition of symptoms. Appropriate dosages for use in a particular individual, for example in human patients, may then be more accurately determined using such information.

Measures of compounds in plasma may be routinely measured in an individual such as a patient by techniques such as high performance liquid chromatography (HPLC) or gas chromatography.

Pharmaceutical compositions for use in accordance with the present invention may be formulated in conventional manner using one or more physiologically acceptable carriers or excipients.

Thus, the compounds and their physiologically acceptable salts and solvates may be formulated for administration by inhalation or insufflation (either through the mouth or the nose) or oral, buccal, parenteral or rectal administration.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the compositions may take the form of tablets or lozenges formulated in conventional manner. For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

### EXAMPLES

The present invention is also described by means of particular examples. However, the use of such examples anywhere in the specification is illustrative only and in no way limits the scope and meaning of the invention or of any exemplified term. Likewise, the invention is not limited to any particular preferred embodiments described herein. Indeed, many modifications and variations of the invention will be apparent to those skilled in the art upon reading this specification and can be made without departing from its spirit and scope. The invention is therefore to be limited only by the terms of the appended claims along with the full scope of equivalents to which the claims are entitled.

### Example 1

### SOS Promotes Dimerization of FGF-FGFR Complexes

This example describes experiments that were performed in vitro to test whether sucrose octasulfate (SOS) can act as a heparin mimetic. Specifically, the data obtained from these experiments demonstrate that SOS is able to promote the dimerization of complexes between fibroblast growth factor receptors and their ligands (i.e., FGF-FGFR complexes).

A construct encoding an extracellular ligand binding portion of the FGFR1 polypeptide set forth in FIG. 1A (SEQ ID NO:1) was expressed in E. coli and refolded in vivo using established protocols, as previously described by Plotnikov et al. (Cell 2000, 101:413-424). In particular, the soluble FGFR1 polypeptide expressed by this construct, which is referred to here as D23, comprises amino acid residues 142 to 365 of SEQ ID NO:1, which correspond to the immunoglobulin (Ig)-like domains 2 and 3 (D2 and D3, respectively), which are known to confer ligand binding and specificity for the FGFR receptor. However, the D23 polypeptide is missing the Ig-like domain 1 (D1), the acid box and the linker polypeptide sequence between D3 and the transmembrane helix. The D23 polypeptide is therefore similar to a naturally occurring splice variant of FGFR1 that retains full ligand binding capacity (Johnson et al., Mol. Cell. Biol. 1990, 10:4728-4736).

When expressed in E. coli cells, the D23 polypeptide was found entirely in inclusion bodies. The polypeptide was solubilized using standard denaturing reagents and refolded in vitro. Following purification by ion exchange chromatography, the D23 polypeptide was complexed with the FGF2 ligand polypeptide whose amino acid sequence is set forth in FIG. 2A (SEQ ID NO:3) and purified by size exclusion chromatography.

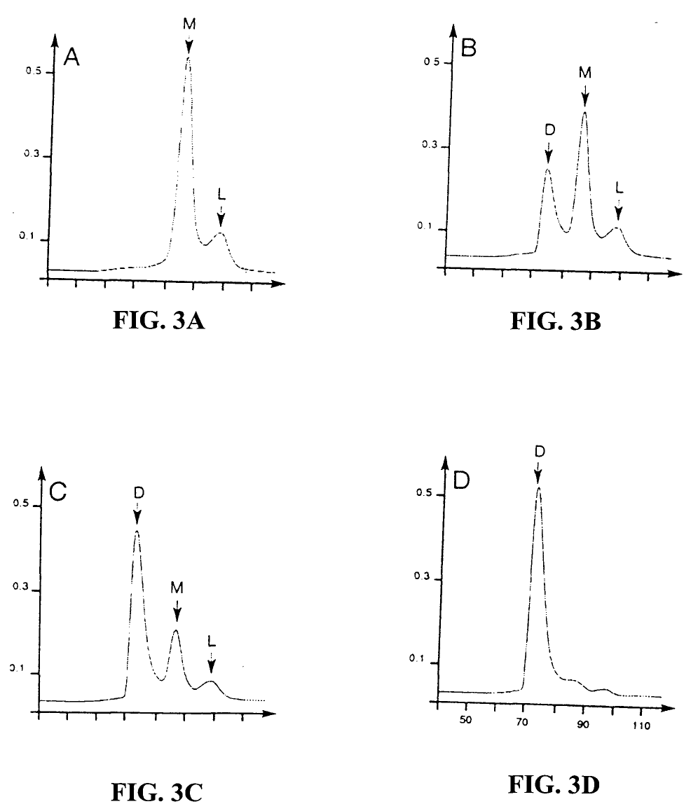

To quantitate dimerization, the purified 1:1 FGF2:FGFR1 complexes were mixed at various molar ratios with SOS and analyzed by size exclusion chromatography according on SUPERDEX 200® (Amersham Pharmacia Biotech.) size exclusion column in 25 mM HEPES-NaOH buffer (pH 7.5) containing 150 mM sodium chloride. The resulting chromatograms are shown in FIGS. 3A-C.

In the absence of SOS (FIG. 3A) only a peak corresponding to monomers of the FGF:FGFR complexes are observed, which is indicated by the letter M. A small peak, identified in FIG. 3A by the letter L, was also observed at higher elution volumes. This peak corresponds to free FGF ligand polypeptide that dissociates from the FGF:FGFR complex due to protein dilution during the chromatography process. As SOS is added to the mixture (FIGS. 3B-3C), a third peak corresponding to dimers of the FGF:FGFR complex is observed (identified by the letter D) while the intensity of the monomer peak (M) decreases. The intensities of the dimer and monomer peaks increase and decrease, respectfully, as SOS is added in higher amounts (compare, e.g., FIG. 3B to FIG. 3C). Finally, when SOS is added at a 1:1:1 molar ratio to the FGF and FGFR (FIG. 3D), only a peak corresponding to FGF:FGFR dimers is observed.

Similar results have also been obtained by the inventors in size exclusion chromatography experiments that used a homogenously sulfated heparin hexasacharide instead of a SOS (see, in particular, Schlessinger et al., Molecular Cell 2000, 6:743-750). However, the results presented here show that small molecules, including sulfated discharides such as SOS, can dimerize an FGF receptor.

### Example 2

### SOS Promotes Activation of the FGF Receptor by FGF in Cells

This example describes experiments that investigated the ability of SOS to modulate FGF ligand-dependent activation of the FGF receptor in vivo. In particular, an assay is described here that uses a BaF3 cell line which overexpresses FGFR1. This cell line has been previously described and is therefore known in the art (see, e.g., Huang et al., J. Biol. Chem. 1995, 270:5065-5072).

BaF3 cells are a lymphoid cell line, which are dependent on interleukin-3 (IL-3) for growth. Ordinarily, these cells do not exhibit any response to FGF. However, when stably transfected to express an FGF receptor, the cells exhibit a dose-dependent mitogenic response to FGF ligand in the absence of IL-3. Accordingly, the growth rate of such transfected cells is useful as a measurement of FGF receptor activity in vivo. Because BaF3 cells express only low amounts of HSPG, soluble heparin must also be present to elicit the FGF-dependent mitogenic response observed in the transfected cells.

For the experiments discussed here, BaF3 cells that stably expressed wild-type FGFR1 (SEQ ID NO:1) were cultured according to standard methods that have been previously described (see, Huang et al., supra). 1×10⁴ cells were seeded in triplicate wells and grown in the presence of heparin (3 μM) or, alternatively, in the presence of various concentrations (0.1, 0.5, 1, 5 and 10 μM, respectively) of SOS. The numbers of viable cells in each well were counted daily in duplicate.

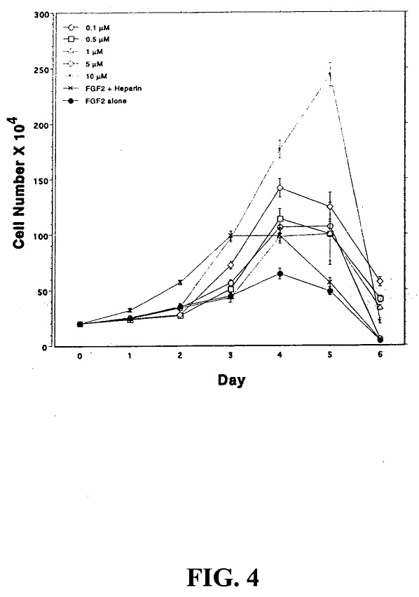

Data from these experiments are graphically presented here in FIG. 4 as mean and standard deviation values. As can be seen from inspecting the figure, SOS supports FGF2 in inducing proliferation of the BaF3 cells over expressing FGFR1 in a dose-dependent manner. As anticipated, the BaF3 cells grow minimally in the presence of FGF2 alone.

Thus, these data complement data from the in vitro experiments presented in Example 1, supra. In particular, these experiments demonstrate not only that SOS can bind to and/or support dimerization of FGF ligand-receptor complexes, but also show that SOS can increase FGF receptor activity in cells, and thereby enhance signaling by an FGF ligand.

### Example 3

### Crystallography of an FGF-FGFR Complex with SOS

This example describes x-ray crystallography experiments that better characterize the molecular mechanisms by which SOS may interact with and/or stabilize dimers of FGF-FGFR complexes. In particular, this example describes the crystalization of FGF2-FGFR1 complexes with SOS and the solution of that crystal structure by analyzing x-ray diffraction data.

Crystals of dimeric FGF2-FGFR1-SOS complexes were grown by vapor diffusion at 20° C. using the hanging drop method. 2 μL of protein solution (10 mg/mL in 25 mM HEPES-NaOH (pH 7.5) and 150 mM NaCl) was mixed with an equal volume of crystallization buffer (12-16% Polyethylene glycol 5000, 0.2 M ammonium sulfate and 15% glycerol in 0.1 M HEPES-NaOH (pH 7.5)). The protein solution contained a 1:1:1 stoichiometric ratio of FGF2, and soluble FGFR1 construct described, supra, in Example 1, and SOS.

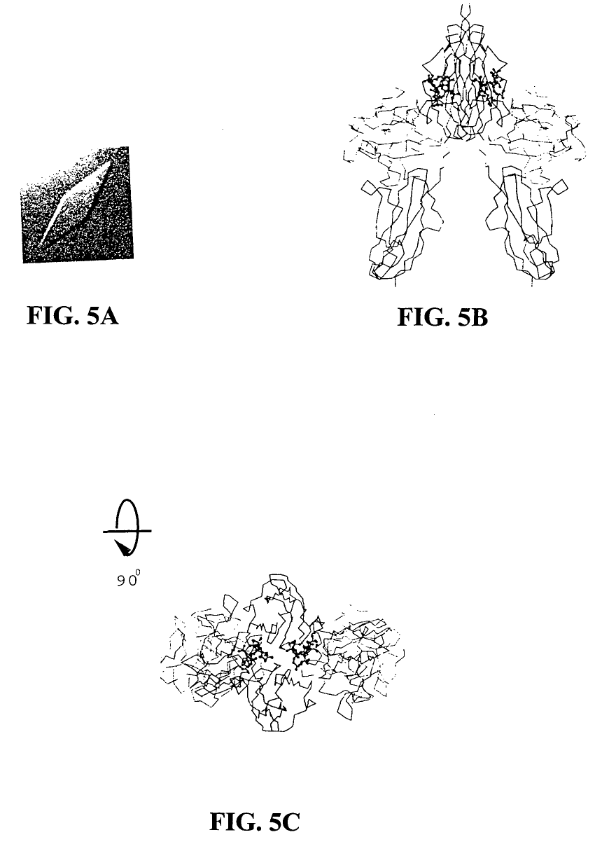

The resultant crystals are shown in FIG. 5A. The crystal belongs to the orthorhombic space group P2₁ 2₁ 2₁ and has unit cell dimensions of a=64.2 Å, b=122.4 Å and c=219.5 Å. The crystal contains four FGF2-FGFR1-SOS complexes in the asymmetric unit with a solvent content of about 56%.

Diffraction data were collected from a flash-frozen crystal on a CCD detector at beamline X4A at the National Synchrotron Light Source, Brookhaven National Laboratory. The data were processed using DENZO and SCALEPACK (Otwinowski & Minor, Methods Enzymol. 1997, 276:307-326). A molecular replacement solution was found for the four copies of the ternary FGF2-FGFR1-SOS complex in the asymmetric unit using the program AmoRe (Navaza, Acata. Crystallogr. Sect. A 1994, 50:157-163) and the binary FGF2-FGFR1 crystal structure deposited in the Protein Data Bank (see, Berman et al., Nucl. Acids Res. 2000, 28:235-242) under ID code 1CVS (Plotnikov, Cell 1999, 98:641-650) as the search model.

The initial model for the structure of SOS was taken from the FGF 1-SOS crystal structure deposited in the Protein Data Bank under ID code 1 AFC (Zhu et al., Structure 1993, 1:27-34). Parameters for the SOS molecule were generated using the HIC-Up server (Kleywegt & Jones, Acta. Crystallogr. D 1998, 54:1119-1131). The models were refined by simulated annealing and positional/B-factor refinement using CNS (Brunger et al., Acta Crystallogr. Sect. D 1998, 54:905-921) with bulk solvent and anisotropic B-factor corrections applied. Tight noncrystallographic symmetry restrains were imposed throughout the refinement for the backbone atoms of FGF2 domains D2 and D3. Model building into the 2Fₒ−F_{c} and Fₒ−F_{c} electron density maps was performed with the program O (Jones et al., Acta Crystallogr. Sect. A 1991, 47:110-119).

From these methods, the crystal structure has been refined to a 2.6 Å resolution with an R value of 24% (free R value of 28%). The atomic model consists of four FGF2 molecules (residues 16 to 144 from SEQ ID NO:1), four FGFR2 molecules (residues 149 to 359 from SEQ ID NO:3), four SOS molecule, three sulfate ions and 42 molecules of water. A list of coordinates for the final structure is provided here, in PDB file format, at the Appendix infra. Data collection and refinement statistics are given in Table 1, below.

TABLE 1
Summary of crystallographic analysis
I. Data Collection Statistics:

| | Reflections | | | |
|---|---|---|---|---|
| Resolution (Å) | (total/unique) | Completeness (%) | R_{sym}^{a} (%) | Signal (<\|σ⁻¹>) |
| 30.0-2.6 | 764014/53698 | 99.9 (100.0)^{b} | 7.8 (33.2)^{b} | 13.5 |

II. Refinement Statistics:^{c}

| | | | Root-mean-square Deviations | | |
|---|---|---|---|---|---|
| Resolution (Å) | Reflections | R_{cryst}/R_{fee}^{d} (%) | Bonds (Å) | Angles (°) | B-factors^{e} (Å) |
| 25.0-2.6 | 52014 | 24.1/27.8 | 0.008 | 1.4 | 1.00 |

${}^{a} {R}_{sym} = 100 \times {\sum }_{hkl} {\sum }_{i} \vert {l}_{i} ( hkl ) - < ( hkl ) > \vert / {\sum }_{hkl} {\sum }_{i} {l}_{i} ( hkl ) .$
^{b}Value in parentheses is for the highest resolution shell: 2.69-2.6 Å.
^{c}Atomic model: 10823 protein atoms, 4 SOS molecules, 3 SO₄²⁻ ions and
42 water molecules.
${}^{d} {R}_{cryst / free} = 100 \times {\sum \vert \vert}_{hkl} {F}_{o} ( hkl ) \vert - \vert {F}_{c} ( hkl ) \vert \vert / {\sum }_{hkl} \vert {F}_{o} ( hkl ) \vert ,$
where Fₒ (>0σ) and F_{c} are the observed and calculated
structure factors. 5% of the reflections were used for calculations of R^{free}.
^{e}For bonded protein atoms.

### Example 4

### Analysis of the Dimerized FGF-FGFR-SOS Crystal Structure

Coordinates for the Final Refined Crystal Structure of the FGF-FGFR Dimer complex with SOS is provided here, in PDB format, in the accompanying Appendix.

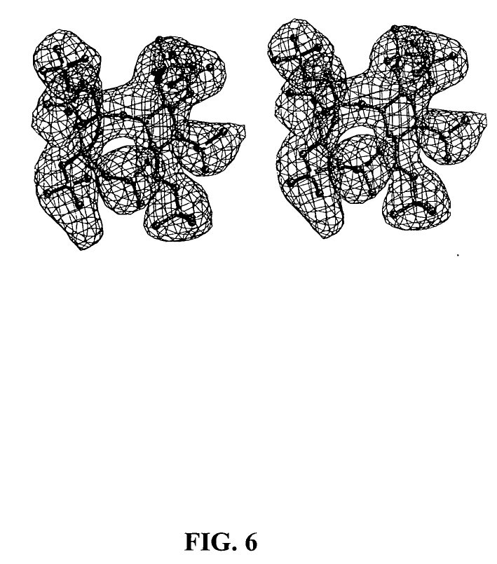

Description of the overall structure. The four 1:1:1 FGF2-FGFR:SOS complexes of the crystals' asymmetric unit are arranged into two dimeric assemblies. Each dimer structure closely resembles the dimeric assembly of the binary FGF2-FGFR1 complexes describes previously by Plotnikov et al. (Cell 1999, 98:641-650), and may be viewed conceptually as the association of two 1:1:1 ternary complexes of FGF2:FGFR2:SOS. The structure of the FGF2:FGFR2:SOS dimers was visualized using the Molscript and Raster3D programs (see, Kraulis, J. Appl. Crystallogr. 1991, 24:946-950; and Merritt & Bacon, Methods Enzymol. 1997, 277:505-524). The overall structure for one dimer complex is illustrated in FIG. 5B. The same structure is also illustrated in FIG. 5C, as viewed when the structure illustrated in FIG. 5B is rotated 90° around the horizontal axis. The Fₒ−F_{c} electron density map computed after simulated annealing with SOS omitted from the atomic model was also visualized using the Bobscript program (see, Esnouf, J. Mol. Graph. Model 1997, 15:132-134), and is shown in FIGS. 6A-B.

Within each ternary complex, the FGF2 ligand binds to the D2 and D3 domains of the receptor FGFR1, as well as to the linker sequence between the D2 and D3 domains of FGFR1. The dimer, in turn, is held together by interactions of the FGF2, FGFR1 and SOS from one ternary complex with the FGFR1 in the other, adjoining ternary complex within the dimer.

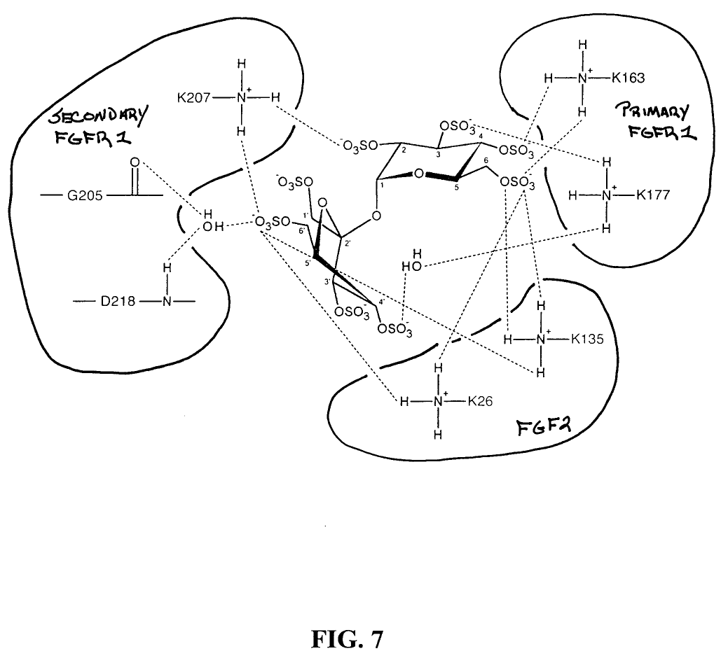

The SOS binding site. Each dimer in the crystals' asymmetric unit contains two SOS molecules, which bind to the same general region of the FGF-FGFR1 dimer complex that has been shown to bind heparin (see, Schlessinger et al., Molecular Cell 2000, 6:743-750). As can be seen in FIG. 6, the Fₒ−F_{c} electron density for one of the SOS molecules is strong and well contoured, while the density for the second SOS molecule is less defined, indicating that this second SOS molecule is somewhat less ordered within the crystals. The well ordered SOS molecule makes a total of 13 hydrogen bonds with on FGF2 and both FGFR1 molecules in the asymmetric unit. These H-bonds, which are illustrated in FIG. 7, stabilize the FGF2-FGFR1 complexes, and also promote dimerization.

Interactions of SOS with FGF and FGFR in the dimer. Within each ternary complex, SOS makes five hydrogen bonds with FGF2 and four with FGFR1. These hydrogen bonding interactions are illustrated schematically in FIG. 7. Specifically, hydrogen bonding interactions are observed between both the 5- and 6-membered rings of SOS and Lysines 163 and 177 of FGFR1. These lysines are located on the heparin binding surface of the D2 domain in FGFR1, and have also been shown to bind heparin in the crystal structure of a FGF2-FGFR1 complex with heparin (see, Schlessinger et al., Molecular Cell 2000, 6:743-750).

SOS also interacts with the D2 domain of the FGFR molecule in the adjoining ternary complex of the crystals' asymmetric unit. Specifically, a hydrogen bond is observed between Lysine 207 of the second FGFR molecule and the 2-sulfate (in the 6-membered ring) of SOS. Another hydrogen bond is observed between Lysine 207 of the second FGFR molecule and the 6′-sulfate (in the 5-membered ring) of SOS. Interestingly, Lysine 207 has also been implicated in heparin binding (see, Schlessinger et al., supra). Two addition hydrogen bonds, mediated by a water molecule, are observed between the 6′-sulfate of SOS and backbone atoms in the glycine 205 and aspartic acid 218 amino acid residues of the second FGFR molecule.

Five additional hydrogen bonds are made between Lysines 26 and 135 of FGF2 and the sulfate groups of SOS. In the crystal structure of a ternary FGF2-FGFR1-heparin complex described by Schlessinger et al., supra, these FGF2 lysines form hydrogen bonds to heparin.

Thus, the crystal structure described here demonstrates that SOS interacts with FGF and FGFR in a way that mimics the proteins' reaction with heparin, and similarly increases FGF-FGFR binding affinity.

### Example 5

### Heparin Agonists and Antagonists as Therapeutic Agents

The experiments described in Examples 1-4, supra, demonstrate that SOS can interact with an FGF ligand and/or its receptor and, moreover, that this interaction enhances dimerization of the receptor-ligand complex, and increases receptor activity. Recent biochemical and structural data have indicated that FGF may form an initial, low affinity complex with FGFR in the absence of heparin (see, e.g., Pantoliano et al., Biochemistry 1994, 33:10229-10248; and Plotnikov et al., Cell 1999, 98:641-650). However, this minimal 1:1 complex may, at best, only allow transient receptor dimerization and signaling at high, non-physiological concentrations of the receptor and/or its ligand. Under normal physiological concentrations, the FGF ligand and its receptor tend to dissociate, and do not have sufficient oportunity to interact simultaneously with a second FGF receptor. Without being bound to any particular theory or mechanism of action, it is therefore believed that the presence of either heparin or SOS is necessary under normal physiological concentrations of FGF ligand and/or receptor to stabilize the low affinity receptor-ligand complexes, and provide sufficient opportunity for the concerted binding of FGF ligand and receptor in one monomeric ternary complex to the FGFR in a second monomeric ternary complex. In other words, both heparin and SOS are believed to bind to FGF ligand and receptor and generate stable receptor-ligand complexes which, in turn, provide sufficient interface for the binding of a second FGF receptor molecule.

The crystal structures described in Example 4, supra, provide, for the first time, specific interactions that stabilize an FGF ligand-receptor complex and, moreover, additional interactions between SOS and a second FGF receptor which stabilize dimerization. The results presented in these example therefore provide an excellent framework for the development of novel therapeutic agents. The discovery is particularly useful in view of the current limitations in large-scale preparation of homogenous heparin oligosaccharides for therapeutic purposes (see, Pervin et al., 1995). In contrast, total de novo synthesis of homogenously sulfated sucrose derivatives is straightforward and known in the art. See, for example, Vlahov et al., J. Carbohydr. Chem. 1997, 16:1-10; Polat et al., J. Carbohydr. Chem. 1997, 16:1319-1325; and Bazin et al., Carbohydr. Res. 1998, 309:189-205. Exemplary, non-limiting examples of such therapeutic compounds are described here, along with some particular examples of their utility as therapeutic agents.

Heparin antagonists. Compounds that may be used as therapeutic agents of the present invention include ones that function or are likely to function as heparin antagonist by competing with heparin to sequester FGF-FGFR complexes in a “signaling-incompetent” state. In particular, preferred therapeutic compounds of the invention include suramin and derivatives of sucrose octasulfate (SOS) that retain SOS's ability to generate stable FGF-FGFR complexes while, at the same time, inhibiting dimerization or signaling ability of those complexes. Example 5, described supra, demonstrates that suramin can interact with a pre-formed FGF ligand-receptor complex, thereby stabilizing the interaction, while inhibiting signaling through the FGF receptor. Other exemplary heparin antagonists of the invention include derivatives of compounds such as inositol hexasulfate and sulfated β-cyclodextrin, as well as derivatives of other compounds that behave in an analogous manner to SOS and promote signaling competent dimers of the FGF ligand and receptor. As with heparin antagonists that are derivatives of SOS, heparin antagonists that are derivatives of some other compound (e.g., inositol hexasulfate or sulfated β-cyclodextrin) have the ability to generate stable FGF-FGFR complexes while, at the same time, inhibiting dimerization of those complexes. Thus, preferred heparin antagonists are compounds that generate stable, dimerization incompetent complexes of FGF-FGFR.

In one preferred embodiment, heparin antagonists of the invention include SOS derivatives having one or more substitutions of sulfates that are involved in stabilizing interactions between a first FGF-FGFR complex and a second FGF receptor. Specific examples of such substitutions, that are particularly preferred, including substitutions at either the 2- and/or the 1′ positions of SOS. Preferred substitutions include, but are not limited to, substitutions of a bulky group such as a benzyl, benzoyl, pivaloyl, fatty acyl, trityl or isopropylidene moiety for one or more sulfate moieties. However, any moiety that may be reasonably expected to block or inhibit hydrogen bonding interactions between SOS and FGFR which stabilize dimerization may be used as a substituent.

In another preferred embodiment, the heparin antagonist of the invention is suramin, a polysulfonated napthylurea that induces dimerization of pre-formed binary FGF2-FGFR1 complexes that are signaling incompetent. Without being limited to a particular mechanism or theory, the non-productive dimers may be a result of nonproductive spatial positioning of the FGFR D3 regions in the dimeric assemblies. However, the preliminary data presented in Example 5, supra, cannot exclude other potential models.

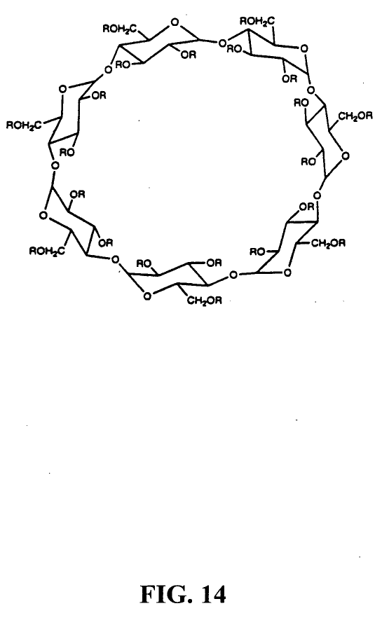

In yet another preferred embodiment, heparin antagonists of the invention include sulfated derivatives of a cyclodextrin compound such as sulfated derivatives of α-cyclodextrin, β-cyclodextrin and γ-cyclodextrin. Cyclodextrin compounds are known in the art (see, for example, Hileman et al., Electrophoresis 1998, 19(15):2677-2681). The compounds are generally defined as a cyclic ring of 1→4 linked glucose residues. A general structural formula for derivatives of a preferred cyclodextrin, β-cyclodextrin, is provided in FIG. 14 (Structure VIII).

Cyclodextrin compounds are typically classified based on the number of 1→4 linked glucose residues present in the ring, with rings of between 6 and 12 glucose residues being preferred. Rings of 6, 7 and 8 glucose residues are particularly preferred. Thus, cyclodextrin compounds that comprise a ring of six 1→4 linked glucose residues (i.e., n=6) are referred to as α-cyclodextrin compounds. Cyclodextrin compounds that comprise a ring of seven 1→4 linked glucose residues are referred to as β-cyclodextrin compounds (FIG. 14, Structure VHI) and cyclodextrin compounds that comprise a ring of eight 1→4 linked glucose residues are referred to as γ-cyclodextrin compounds. Referring to the general structure provided in FIG. 14 (Structure VIII), each of the group labeled “R” on each of the glucose residues is generally a hydrogen. However, other chemical moieties may be substituted for these groups to form cyclodextrin derivative compounds, such as sulfated cyclodextrin or sulfonated cyclodextrins.

Preferred cyclodextrin compounds that are heparin antagonists are sulfated cyclodextrin. Each group R on each of the glucose residues in a sulfated cyclodextrin preferably is independently a hydrogen (H) or a sulfate group (SH). At least one sulfate group must be present. However, it is more preferably that at least about 50% or more (e.g., at least 60%, 70%, 80%, 90%, 95%, 99% or 100%) of the cyclodextrin hydroxyl residues is sulfated. Generally, a sulfated cyclodextrin molecule used in the methods and compositions of the present invention may comprise a mixture of sulfated cyclodextrin molecules, with each molecule preferably comprising the same number of glucose residues in the cyclodextrin ring but having different hydroxyl residues and/or different numbers of hydroxyl residues substituted with a sulfate group.

Heparin antagonists, such as the ones described hereabove, are expected to inhibit dimerization or signaling of an FGF receptor and therefore decrease FGFR mediated signaling. Such compounds may be useful, therefore, as agents for inhibiting biological activities associated with FGFR signaling or activity including, for example, angiogenesis and tumor growth.

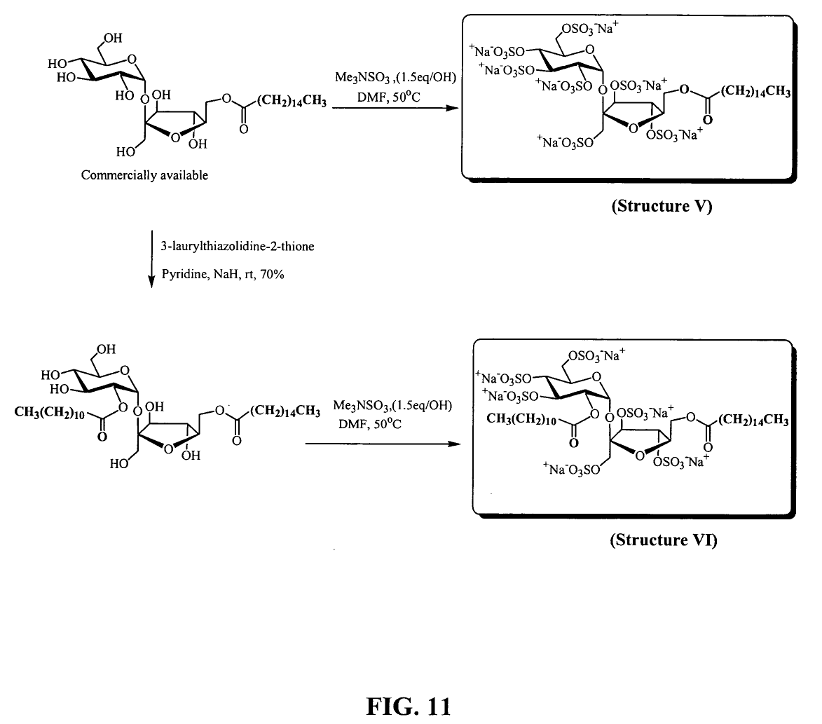

FIGS. 8-11 illustrate the exemplary synthesis of six other preferred SOS derivatives (structures I, II III, IV, V and VI) that may be used as heparin antagonists in the present invention. For example, in one preferred embodiment the SOS derivative may be 2-O-Bn sucrose heptasulfate (structure I). In another preferred embodiment an SOS derivative may be 1′-O-Bn sucrose heptasulfate (structure II). In yet another preferred embodiment, an SOS derivative of the invention may be 1′,2-di-O-Bn sucrose hexasulfate (structure III). Other preferred, exemplary SOS derivatives of the invention may include 4,6-O-isopropyliden sucrose hexasulfate (Structure IV), 6′-O-hexadecanoyl sucrose heptasulfate (Structure V) and 2-)-dodecanoyl, 6′-O-hexadecanoyl sucrose hexasulfate. Still other compounds, including other SOS derivatives, which may be used in the methods of this invention will be readily apparent to those skilled in the art given what is taught in this specification. Such compounds may also be readily synthesized by chemical reactions such as the ones illustrated in FIGS. 8 through 11 that are routine and well known in the art (see, for example, Pervin et al., Glycobiology 1995, 5:83-95; Desai et al., Carbohydr. Res. 1995, 275:391-401; Vlahov et al., J. Carbohydr. Chem. 1997, 16:1-10; Polat et al., J. Carbohydr. Chem. 1997, 16:1319-1325; Bazin et al., Carbohydr. Res. 1998, 309:189-205; Jenner & Khan, J.C.S. Chem. Comm. 1980, pp. 50-51).

Heparin agonists. Compounds that may be used in the methods of this invention further include ones that function or are likely to function as heparin agonists. In particular, the compounds of the present invention include derivatives of sucrose octasulfate (SOS) and other compounds that enhance or promote the dimerization of FGF receptor-ligand complexes. Other exemplary heparin agonists of the invention include compounds such as inositol hexasulfate, sulfonated β-cyclodextrin, and derivatives thereof that enhance or promote the dimerization of FGF receptor-ligand complexes.

Generally, such compounds can be identified by those skilled in the art as having stabilizing interactions (for instance, hydrogen bonding interactions) in an FGF-FGFR dimer structure that preserve the stabilizing interactions observed in the FGF-FGFR dimer structure described in the above Examples. Indeed, those skilled in the art will appreciate that compounds which may be used as heparin agonists in the present invention may even have stabilizing interactions that are stronger than, or at least similar to, those in the FGF-FGFR-SOS ternary complex structures described here.

The examples, supra, demonstrate that compounds such as SOS and derivatives thereof may effectively function as heparin agonists, and effectively increase cell signaling activities mediated by an FGF ligand and/or its receptor. Thus, such compounds are useful for increasing activities that are associated with FGF signaling including, for example, tyrosine kinase activity and angiogenesis. Such compounds are particularly useful in applications where it is desirable to promote a biological activity stimulated by FGF signaling. For example, in one preferred embodiment a heparin agonist may be used to promote wound healing in an individual, e.g., by promoting mitogenic activity. In other preferred embodiments, heparin agonists of the invention (for example, sulfated inositols and sulfated β-cyclodextrins) may be used to treat disorders such as stomach ulcers by promoting dimerization of an FGF receptor-ligand complex.

In particularly preferred embodiments, heparin agonists of the invention include sulfonated derivatives of a cyclodextrin compound, including sulfonated derivatives of α-cyclodextrin, β-cyclodextrin and γ-cyclodextrin. For instance, Example 7, infra, describes experiments demonstrating that sulfonated β-cyclodextrin is an effective heparin agonist.

Cyclodextrin compounds are described, supra, in connection with preferred heparin antagonists of this invention and a general structural formula for a derivatives of a preferred cyclodextrin, β-cyclodextrin, is provided in FIG. 14 (Structure VIII). Preferred cyclodextrin compounds that are heparin agonists are sulfonated cyclodextrins. Each group R on each of the glucose residues of a sulfonated cyclodextrin preferably is independently a hydrogen (H) or a sulfonate group (SO₃), although other substituents may also be present. At least one sulfonate group must be present. However, it is more preferable that at least about 50% or more (e.g., at least 60%, 70%, 80%, 90%, 95% or 100%) of the cyclodextrin hydroxyl residues is sulfonated. Generally the sulfonated cyclodextrin molecules used in the methods and compositions of the present invention may comprise a mixture of sulfonated cyclodextrin molecules, with each molecule preferably comprising the same number of glucose residues in the cyclodextrin ring but having different hydroxyl residues and/or different numbers of hydroxyl residues substituted with a sulfonate group.

### Example 6

### Suramin Promotes Formation of FGFR Dimers that are Signal Incompetent

This example describes experiments that investigate the ability of another compound, suramin, to modulate FGF ligand-dependent activation of an FGF receptor. Specifically, the data presented in this example demonstrates that suramin can interact with FGF receptor-ligand complexes, and promotes dimerization of the FGF receptor. Unlike SOS, however, the FGFR dimers formed with suramin are actually signaling incompetent. Thus, these examples demonstrate an alternative mechanism by which certain compounds, including suramin, may act as agonists or FGF-mediated signaling.

Suramin is a polysulfonated napthylurea with has the chemical structure set forth in FIG. 12 (Structure VII). The compound has demonstrated anti-tumor activity against a variety of different types of cancers, including breast cancer, prostate cancer, sarcoma, colorectal cancer, Karposi's sarcoma, non-Hodgikin's lymphoma, renal cell carcinoma and adrenal carcinoma to name a few. See, for example, Voogd et al., 1993; La Rocca et al.). The compound's anti-tumor activity may be due to an ability to bind to and inhibit FGF (see, Takano et al., 1994; Waltenberger et al., 1996). Indeed, suramin has been demonstrated to bind an FGF 1 ligand and induce its aggregation (Middaugh et al., 1992). At present, however, no structural data are available to indicate how suramin might interact with an FGF ligand or receptor.

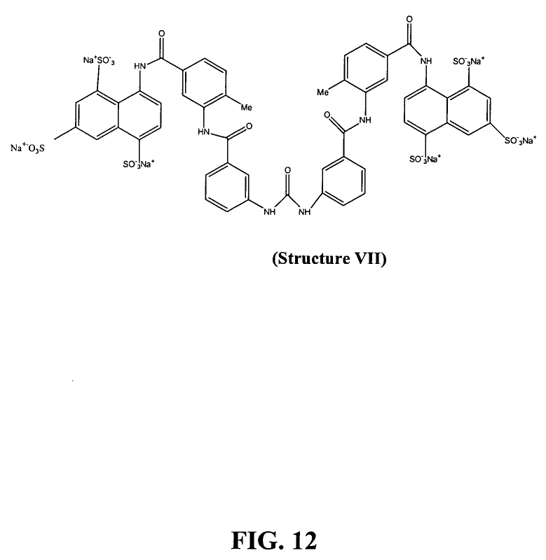

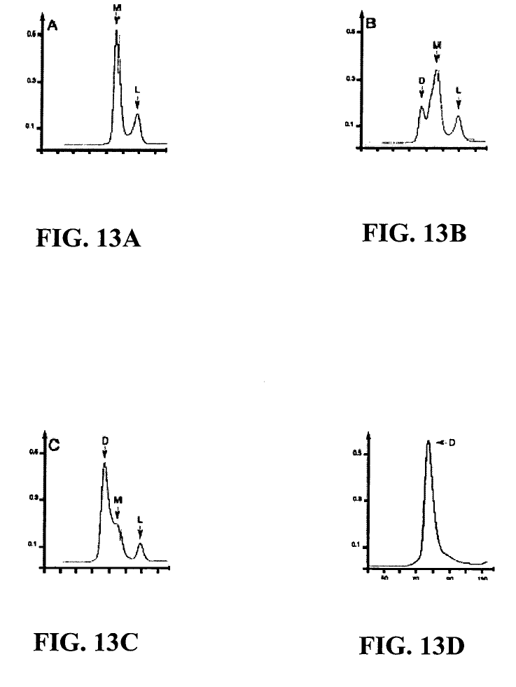

In these experiments, two milligram aliquots of the purified FGF2-FGFR1 complex described, supra, in Example 1 were mixed with suramin and analyzed on a size exclusion column equilibrated with 25 mM HEPES-NaOH buffer (pH 7.5) containing 150 mM NaCl. The resulting chromatograms are shown in FIGS. 13A-13D.

In the absence of suramin (FIG. 13A), only a peak corresponding to monomers of the FGF:FGFR complex are observed, which is indicated by the letter M. A small peak, identified in FIG. 13A by the letter L, was also observed at higher elution volumes. This peak corresponds to free FGF ligand polypeptides that dissociates from the FGF:FGFR complex due to protein dilution during the chromatography process. As suramin is added to the mixture (FIGS. 13B-13C) a third peak corresponding to dimers of the FGF:FGFR complex is observed (identified by the letter D) while the intensity of the monomer peak (M) decreases. The intensities of the dimer and monomer peaks increase and decrease, respectively, as suramin is added in higher amounts (compare, e.g., FIG. 13B to FIG. 13C). Finally, when suramin is added at a 1:1:1 molar ratio to FGF and FGFR (FIG. 13D) only a peak corresponding to the FGF:FGFR dimers is observed. Thus, these experiments yield the surprising result that suramin can bind to and promote dimerization of preformed FGF-FGFR complexes.

Paradoxically, however, FGFR dimers promoted by suramin are signaling incompetent. That is to say, the FGF receptor is not activated in these dimers. To demonstrate this property, experiments that are essentially identical to those described, supra, in Example 2 were performed to investigate suramin's ability to modulate FGF ligand-dependent activation of the FGF receptor in vivo. However, in these experiments, BaF3 cells were grown in the presence of suramin, rather than heparin of SOS, and contacted with FGF ligand. However, no heparin-like or SOS-like activity was observed when these cells were cultured with suramin.

### Example 7

### Sulfonated Cyclodextrin Promotes Activation of the FGF Receptor by FGF in Cells

This examples describes experiments that investigate the ability of sulfonated β-cyclodextrin to function as an effective heparin agonists. In particular, the cell-based assay described in Example 2, supra, is used here to investigate the ability of sulfonated β-cyclodextrin to modulate FGF ligand-dependent activation of the FGF receptor in vivo.

The assay uses a BaF3 cell line which overexpresses FGFR1. This cell line has been previously described and is known in the art (see, e.g., Huang et al., J. Biol. Chem. 1995, 270:5065-5072). BaF3 cells are a lymphoid cell line, which are dependent on interleukin-3 (IL-3) for growth. Ordinarily these cells do not exhibit any response to FGF. However, when stably transfected to express an FGF receptor, the cells exhibit a dose-dependent mitogenic response to FGF ligand in the absence of IL-3. Accordingly, the growth rate of such transfected cells is useful as a measurement of FGF receptor activity in vivo. Ordinarily, because BaF3 cells express only low amounts of HSPG, soluble heparin must also be present to elicit the FGF-dependent mitogenic response observed in the transfected cells.

For the experiments described here, BaF3 cells that stably express wild-type FGFR1 (SEQ ID NO:3) were cultured according to standard methods that have been previously described (see, Huang et al., supra). 1×10⁴ cells were seeded in triplicate wells and grown in the presence of FGF1 ligand (50 ng/ml) and heparin (10 μg/ml) or, alternatively, in the presence of various concentrations of sulfonated β-cyclodextrin (1 μM., 5 μM, 10 μM and 25 μM, respectively). The numbers of viable cells in each well were counted daily in duplicate. Control experiments were also performed in which cells were incubated with either FGF1 ligand alone (i.e. no heparin or sulfonated β-cyclodextrin) or in factor-free medium with neither FGF ligand, heparin or cyclodextrin derivatives.

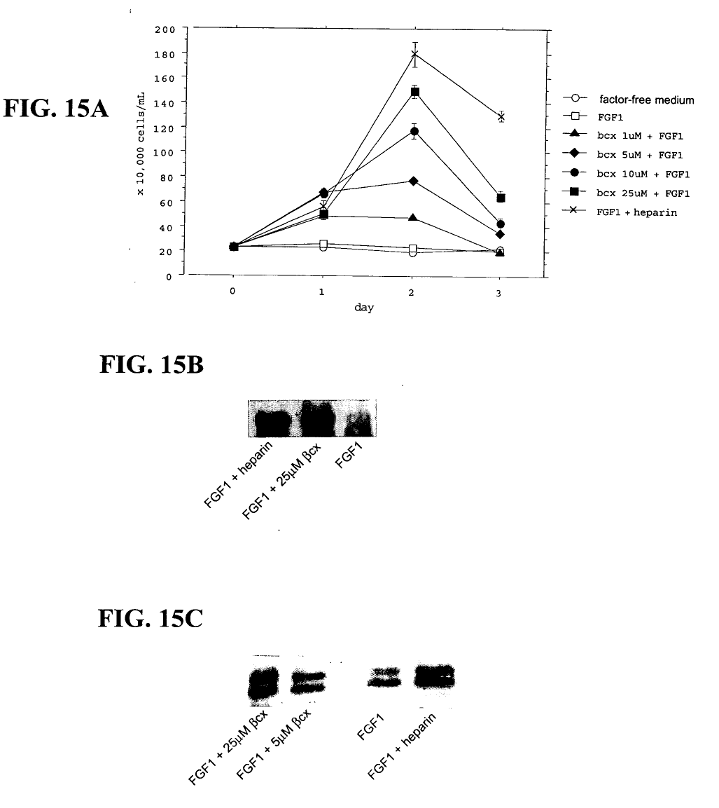

Data from these experiments are graphically presented in FIG. 15A as mean and standard deviation values. As can be seen from inspecting that figure, sulfonated β-cyclodextrin supports the FGF ligand in inducing proliferation of the BaF3 cells over expressing FGFR1 in a dose-dependent manner. As expected, the BaF3 cells grow minimally without FGF ligand or when grown in the presence of FGF ligand alone (i.e., without heparin or sulfonated β-cyclodextrin).

To verify that the effect of sulfonated β-cyclodextrin observed in FIG. 15A is actually due to activation of the FGF receptor, experiments were conducted that examined the capacity of heparin and β-cyclodextrin to stimulate kinase activity of FGF receptor in living cells. See, Mohammadi et al., Science 1997, 276:955-960 for a detailed description of such experiments.

Briefly, BaF3 cells over-expressing FGFR were stimulated for five minutes with FGF1 ligand (50 ng/ml), heparin (10 μg/ml) and/or sulfonated α-cyclodextrin 5 or 25 μM). The cells were then lysed. Their proteins were immunoprecipitated with antibodies to FGFR1, separated by SDS-polyacrylamide gel electrophoresis (PAGE), immunoblotted with antibodies to phosphotyrosine, and detected by autoradiography. As expected, the FGF ligand stimulated autophosphorylation of the FGF receptor when incubated with cells in the presence of heparin, whereas no autophosphorylation of the receptor is observed when the cells are incubated in the presence of FGF1 ligand alone (i.e., with no co-factors). See, the left-hand and right-hand lanes, respectively, in FIG. 15B. Incubation of cells with FGF1 ligand and sulfonated β-cyclodextrin also results in autophosphorylation of the FGF receptor, as illustrated in the middle lane of FIG. 15B.

Co-incubation of the cells with either heparin or sulfonated β-cyclodextrin also induces autophosphorylation of ERK-1 and ERK-2, two intracellular events that are dependent on the kinase activity of FGFR1 (FIG. 15C). By contrast, incubation of the cells with FGF 1 alone (i.e., no co-factor) resulted in no autophosphorylation of either ERK-1 or ERK-2.

Thus, the data from these experiments demonstrate that sulfonated cyclodextrin derivatives are effective heparin agonists and increase FGF receptor activity in cells, thereby enhancing signaling by an FGF ligand.

### References Cited

Numerous references, including patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entirety and to the same extent as if each reference was individually incorporated by reference.

APPENDIX
CRYSTAL STRUCTURE COORDINATES
FOR AN FGF-FGFR-SOS TERNARY COMPLEX

| | |
|---|---|
| REMARK | coordinates from restrained individual B-factor refinement |
| REMARK | refinement resolution: 25 - 2.6 A |
| REMARK | starting r= 0.2409 free_r= 0.2774 |
| REMARK | final r= 0.2408 free_r= 0.2778 |
| REMARK | B rmsd for bonded mainchain atoms= 0.809 target = 1.5 |
| REMARK | B rmsd for bonded sidechain atoms= 1.077 target = 2.0 |
| REMARK | B rmsd for angle mainchain atoms= 1.458 target = 2.0 |
| REMARK | B rmsd for angle sidechain atoms= 1.726 target = 2.5 |
| REMARK | wa= 2.05842 |
| REMARK | rweight = 0.148674 |
| REMARK | target= mlf steps= 30 |
| REMARK | sg= P2(1)2(1)2(1) a= 64.193 b= 122.374 c= 219.490 |
| REMARK | alpha= 90.000 beta= 90.000 gamma= 90.000 |
| REMARK | parameter file 1 : CNS_TOPPAR: protein_rep.param |
| REMARK | parameter file 2 : CNS_TOPPAR: dna-rna.param |
| REMARK | parameter file 3 : CNS_TOPPAR: water_rep.param |
| REMARK | parameter file 4 : CNS_TOPPAR: ion.param |
| REMARK | parameter file 5 : SCR_par.txt |
| REMARK | molecular structure file: 353sos.mtf |
| REMARK | input coordinates: sos_19X.pdb |
| REMARK | anomalous f′ f″ library: anom_se.lib |
| REMARK | reflection file= 353sos.hklt |
| REMARK | ncs= restrain ncs file= ncs.def |
| REMARK | B-correction resolution: 6.0 - 2.6 |
| REMARK | initial B-factor correction applied to fobs: |
| REMARK | B11= 0.444 B22= −18.604 B33= 18.161 |
| REMARK | B12= 0.000 B13= 0.000 B23= 0.000 |
| REMARK | B-factor correction applied to coordinate array B: −0.119 |
| REMARK | bulk solvent: density level = 0.355606 e/A\hat{ }3 |
| REMARK | B-factor= 25.0325 A\hat{ }2 |
| REMARK | reflections with \|Fobs\|/sigma_F < 0.0 rejected |
| REMARK | reflections with \|Fobs\| > 10000 * rms(Fods) rejected |
| REMARK | theoretical total number of ref1. in resol. range: 54084(100.0%) |
| REMARK | number of unobserved reflections (no entry or \|F\| = 0): 2070(3.8%) |
| REMARK | number of reflections rejected: 0(0.0%) |
| REMARK | total number of reflections used: 52014 (96.2%) |
| REMARK | number of reflections in working set: 49400 (91.3%) |
| REMARK | number of reflections in test set: 2614 (4.8%) |
| CRYST1 | 64.193 122.374 219.490 90.00 90.00 90.00 P 21 21 21 |
| REMARK | FILENAME = “sos_19XB.pdb” |
| REMARK | DATE: 02-Jan-01 22:56:51 created by user: mohammad |
| REMARK | VERSION: 0.5 |

| | | | | | | | | | |
|---|---|---|---|---|---|---|---|---|---|
| ATOM | 1 | C | GLY | 15 | 27.348 | 22.092 | 34.405 | 1.00 | 44.79 |
| ATOM | 2 | O | GLY | 15 | 26.719 | 21.151 | 34.910 | 1.00 | 44.68 |
| ATOM | 3 | N | GLY | 15 | 28.399 | 20.552 | 32.735 | 1.00 | 45.47 |
| ATOM | 4 | CA | GLY | 15 | 27.996 | 21.955 | 33.041 | 1.00 | 45.26 |
| ATOM | 5 | N | HIS | 16 | 27.508 | 23.265 | 35.008 | 1.00 | 44.16 |
| ATOM | 6 | CA | HIS | 16 | 26.922 | 23.521 | 36.309 | 1.00 | 44.70 |
| ATOM | 7 | CB | HIS | 16 | 27.347 | 24.898 | 36.823 | 1.00 | 46.71 |
| ATOM | 8 | CG | HIS | 16 | 27.132 | 25.085 | 38.293 | 1.00 | 48.88 |
| ATOM | 9 | CD2 | HIS | 16 | 27.845 | 25.774 | 39.217 | 1.00 | 50.20 |
| ATOM | 10 | ND1 | HIS | 16 | 26.066 | 24.528 | 38.967 | 1.00 | 49.77 |
| ATOM | 11 | CE1 | HIS | 16 | 26.134 | 24.862 | 40.244 | 1.00 | 51.31 |
| ATOM | 12 | NE2 | HIS | 16 | 27.204 | 25.618 | 40.423 | 1.00 | 51.37 |
| ATOM | 13 | C | HIS | 16 | 25.390 | 23.465 | 36.197 | 1.00 | 43.88 |
| ATOM | 14 | O | HIS | 16 | 24.774 | 24.238 | 35.460 | 1.00 | 42.88 |
| ATOM | 15 | N | PHE | 17 | 24.782 | 22.546 | 36.933 | 1.00 | 43.17 |
| ATOM | 16 | CA | PHE | 17 | 23.337 | 22.411 | 36.902 | 1.00 | 42.70 |
| ATOM | 17 | CB | PHE | 17 | 22.890 | 21.409 | 37.974 | 1.00 | 41.54 |
| ATOM | 18 | CG | PHE | 17 | 23.093 | 21.892 | 39.387 | 1.00 | 40.76 |
| ATOM | 19 | CD1 | PHE | 17 | 22.077 | 22.568 | 40.060 | 1.00 | 40.09 |
| ATOM | 20 | CD2 | PHE | 17 | 24.310 | 21.702 | 40.033 | 1.00 | 39.89 |
| ATOM | 21 | CE1 | PHE | 17 | 22.268 | 23.047 | 41.350 | 1.00 | 38.94 |
| ATOM | 22 | CE2 | PHE | 17 | 24.509 | 22.179 | 41.323 | 1.00 | 39.80 |
| ATOM | 23 | CZ | PHE | 17 | 23.483 | 22.855 | 41.982 | 1.00 | 39.26 |
| ATOM | 24 | C | PHE | 17 | 22.638 | 23.763 | 37.103 | 1.00 | 42.76 |
| ATOM | 25 | O | PHE | 17 | 21.564 | 23.987 | 36.554 | 1.00 | 42.49 |
| ATOM | 26 | N | LYS | 18 | 23.255 | 24.653 | 37.885 | 1.00 | 43.04 |
| ATOM | 27 | CA | LYS | 18 | 22.723 | 25.999 | 38.164 | 1.00 | 43.34 |
| ATOM | 28 | CB | LYS | 18 | 23.691 | 26.796 | 39.045 | 1.00 | 44.23 |
| ATOM | 29 | CG | LYS | 18 | 23.573 | 26.664 | 40.536 | 1.00 | 46.77 |
| ATOM | 30 | CD | LYS | 18 | 24.665 | 27.522 | 41.186 | 1.00 | 48.25 |
| ATOM | 31 | CE | LYS | 18 | 24.722 | 27.347 | 42.705 | 1.00 | 49.83 |
| ATOM | 32 | NZ | LYS | 18 | 25.938 | 27.997 | 43.302 | 1.00 | 50.55 |
| ATOM | 33 | C | LYS | 18 | 22.561 | 26.849 | 36.904 | 1.00 | 42.94 |
| ATOM | 34 | O | LYS | 18 | 21.584 | 27.583 | 36.749 | 1.00 | 42.85 |
| ATOM | 35 | N | ASP | 19 | 23.573 | 26.782 | 36.043 | 1.00 | 42.59 |
| ATOM | 36 | CA | ASP | 19 | 23.620 | 27.557 | 34.802 | 1.00 | 41.91 |
| ATOM | 37 | CB | ASP | 19 | 24.889 | 27.254 | 33.989 | 1.00 | 43.71 |
| ATOM | 38 | CG | ASP | 19 | 26.166 | 27.577 | 34.719 | 1.00 | 45.39 |
| ATOM | 39 | OD1 | ASP | 19 | 26.166 | 28.472 | 35.595 | 1.00 | 47.08 |
| ATOM | 40 | OD2 | ASP | 19 | 27.183 | 26.933 | 34.385 | 1.00 | 46.25 |
| ATOM | 41 | C | ASP | 19 | 22.470 | 27.287 | 33.855 | 1.00 | 40.44 |
| ATOM | 42 | O | ASP | 19 | 21.809 | 26.248 | 33.933 | 1.00 | 40.25 |
| ATOM | 43 | N | PRO | 20 | 22.248 | 28.213 | 32.907 | 1.00 | 39.00 |
| ATOM | 44 | CD | PRO | 20 | 23.072 | 29.397 | 32.620 | 1.00 | 38.79 |
| ATOM | 45 | CA | PRO | 20 | 21.182 | 28.083 | 31.914 | 1.00 | 37.50 |
| ATOM | 46 | CB | PRO | 20 | 21.096 | 29.475 | 31.274 | 1.00 | 37.48 |
| ATOM | 47 | CG | PRO | 20 | 22.058 | 30.337 | 32.054 | 1.00 | 38.29 |
| ATOM | 48 | C | PRO | 20 | 21.679 | 27.078 | 30.897 | 1.00 | 36.29 |
| ATOM | 49 | O | PRO | 20 | 22.880 | 26.862 | 30.768 | 1.00 | 35.46 |
| ATOM | 50 | N | LYS | 21 | 20.760 | 26.480 | 30.160 | 1.00 | 35.82 |
| ATOM | 51 | CA | LYS | 21 | 21.140 | 25.515 | 29.155 | 1.00 | 35.99 |
| ATOM | 52 | CB | LYS | 21 | 20.914 | 24.078 | 29.674 | 1.00 | 36.49 |
| ATOM | 53 | CG | LYS | 21 | 21.838 | 23.662 | 30.818 | 1.00 | 37.97 |
| ATOM | 54 | CD | LYS | 21 | 21.583 | 22.233 | 31.306 | 1.00 | 39.15 |
| ATOM | 55 | CE | LYS | 21 | 22.452 | 21.932 | 32.533 | 1.00 | 40.16 |
| ATOM | 56 | NZ | LYS | 21 | 22.361 | 20.529 | 33.055 | 1.00 | 41.59 |
| ATOM | 57 | C | LYS | 21 | 20.340 | 25.727 | 27.884 | 1.00 | 35.42 |
| ATOM | 58 | O | LYS | 21 | 19.281 | 26.346 | 27.904 | 1.00 | 35.74 |
| ATOM | 59 | N | ARG | 22 | 20.872 | 25.229 | 26.774 | 1.00 | 34.75 |
| ATOM | 60 | CA | ARG | 22 | 20.176 | 25.294 | 25.501 | 1.00 | 34.07 |
| ATOM | 61 | CB | ARG | 22 | 21.101 | 25.797 | 24.396 | 1.00 | 35.29 |
| ATOM | 62 | CG | ARG | 22 | 21.343 | 27.292 | 24.405 | 1.00 | 37.78 |
| ATOM | 63 | CD | ARG | 22 | 22.090 | 27.710 | 23.148 | 1.00 | 40.24 |
| ATOM | 64 | NE | ARG | 22 | 23.513 | 27.924 | 23.380 | 1.00 | 43.27 |
| ATOM | 65 | CZ | ARG | 22 | 24.029 | 29.059 | 23.845 | 1.00 | 45.68 |
| ATOM | 66 | NH1 | ARG | 22 | 23.229 | 30.087 | 24.127 | 1.00 | 45.61 |
| ATOM | 67 | NH2 | ARG | 22 | 25.345 | 29.171 | 24.028 | 1.00 | 46.18 |
| ATOM | 68 | C | ARG | 22 | 19.753 | 23.853 | 25.216 | 1.00 | 32.72 |
| ATOM | 69 | O | ARG | 22 | 20.498 | 22.913 | 25.495 | 1.00 | 32.82 |
| ATOM | 70 | N | LEU | 23 | 18.549 | 23.669 | 24.695 | 1.00 | 30.88 |
| ATOM | 71 | CA | LEU | 23 | 18.091 | 22.332 | 24.380 | 1.00 | 29.33 |
| ATOM | 72 | CB | LEU | 23 | 16.691 | 22.110 | 24.936 | 1.00 | 27.76 |
| ATOM | 73 | CG | LEU | 23 | 16.710 | 21.842 | 26.438 | 1.00 | 27.28 |
| ATOM | 74 | CD1 | LEU | 23 | 15.317 | 21.643 | 26.964 | 1.00 | 27.57 |
| ATOM | 75 | CD2 | LEU | 23 | 17.536 | 20.585 | 26.696 | 1.00 | 28.42 |
| ATOM | 76 | C | LEU | 23 | 18.112 | 22.126 | 22.878 | 1.00 | 28.98 |
| ATOM | 77 | O | LEU | 23 | 17.254 | 22.627 | 22.159 | 1.00 | 29.13 |
| ATOM | 78 | N | TYR | 24 | 19.124 | 21.396 | 22.419 | 1.00 | 28.51 |
| ATOM | 79 | CA | TYR | 24 | 19.314 | 21.083 | 21.010 | 1.00 | 28.12 |
| ATOM | 80 | CB | TYR | 24 | 20.804 | 20.847 | 20.769 | 1.00 | 26.44 |
| ATOM | 81 | CG | TYR | 24 | 21.197 | 20.462 | 19.366 | 1.00 | 25.39 |
| ATOM | 82 | CD1 | TYR | 24 | 21.080 | 19.146 | 18.916 | 1.00 | 25.38 |
| ATOM | 83 | CE1 | TYR | 24 | 21.504 | 18.782 | 17.640 | 1.00 | 24.64 |
| ATOM | 84 | CD2 | TYR | 24 | 21.739 | 21.405 | 18.499 | 1.00 | 25.53 |
| ATOM | 85 | CE2 | TYR | 24 | 22.161 | 21.055 | 17.228 | 1.00 | 25.30 |
| ATOM | 86 | CZ | TYR | 24 | 22.045 | 19.746 | 16.806 | 1.00 | 25.20 |
| ATOM | 87 | OH | TYR | 24 | 22.491 | 19.421 | 15.553 | 1.00 | 24.70 |
| ATOM | 88 | C | TYR | 24 | 18.495 | 19.841 | 20.651 | 1.00 | 28.56 |
| ATOM | 89 | O | TYR | 24 | 18.726 | 18.758 | 21.188 | 1.00 | 28.69 |
| ATOM | 90 | N | CYS | 25 | 17.531 | 20.003 | 19.752 | 1.00 | 28.93 |
| ATOM | 91 | CA | CYS | 25 | 16.691 | 18.885 | 19.338 | 1.00 | 29.34 |
| ATOM | 92 | CB | CYS | 25 | 15.371 | 19.407 | 18.786 | 1.00 | 28.57 |
| ATOM | 93 | SG | CYS | 25 | 14.151 | 18.130 | 18.521 | 1.00 | 27.13 |
| ATOM | 94 | C | CYS | 25 | 17.377 | 18.019 | 18.290 | 1.00 | 29.52 |
| ATOM | 95 | O | CYS | 25 | 17.904 | 18.527 | 17.311 | 1.00 | 29.96 |
| ATOM | 96 | N | LYS | 26 | 17.363 | 16.711 | 18.499 | 1.00 | 30.38 |
| ATOM | 97 | CA | LYS | 26 | 17.999 | 15.775 | 17.582 | 1.00 | 31.27 |
| ATOM | 98 | CB | LYS | 26 | 17.907 | 14.363 | 18.157 | 1.00 | 29.40 |
| ATOM | 99 | CG | LYS | 26 | 18.580 | 13.292 | 17.333 | 1.00 | 27.53 |
| ATOM | 100 | CD | LYS | 26 | 18.601 | 11.990 | 18.104 | 1.00 | 25.63 |
| ATOM | 101 | CE | LYS | 26 | 19.451 | 10.965 | 17.421 | 1.00 | 24.68 |
| ATOM | 102 | NZ | LYS | 26 | 18.924 | 10.707 | 16.055 | 1.00 | 25.40 |
| ATOM | 103 | C | LYS | 26 | 17.341 | 15.816 | 16.213 | 1.00 | 33.00 |
| ATOM | 104 | O | LYS | 26 | 17.962 | 15.515 | 15.192 | 1.00 | 34.06 |
| ATOM | 105 | N | ASN | 27 | 16.080 | 16.212 | 16.198 | 1.00 | 34.37 |
| ATOM | 106 | CA | ASN | 27 | 15.319 | 16.276 | 14.964 | 1.00 | 35.49 |
| ATOM | 107 | CB | ASN | 27 | 13.840 | 16.054 | 15.283 | 1.00 | 36.29 |
| ATOM | 108 | CG | ASN | 27 | 13.020 | 15.786 | 14.051 | 1.00 | 37.55 |
| ATOM | 109 | OD1 | ASN | 27 | 13.468 | 15.086 | 13.141 | 1.00 | 37.50 |
| ATOM | 110 | ND2 | ASN | 27 | 11.799 | 16.320 | 14.019 | 1.00 | 37.52 |
| ATOM | 111 | C | ASN | 27 | 15.511 | 17.586 | 14.191 | 1.00 | 35.42 |
| ATOM | 112 | O | ASN | 27 | 14.691 | 18.494 | 14.273 | 1.00 | 35.77 |
| ATOM | 113 | N | GLY | 28 | 16.605 | 17.676 | 13.442 | 1.00 | 35.14 |
| ATOM | 114 | CA | GLY | 28 | 16.860 | 18.865 | 12.657 | 1.00 | 34.17 |
| ATOM | 115 | C | GLY | 28 | 17.881 | 19.807 | 13.257 | 1.00 | 34.08 |
| ATOM | 116 | O | GLY | 28 | 18.360 | 20.707 | 12.581 | 1.00 | 34.14 |
| ATOM | 117 | N | GLY | 29 | 18.211 | 19.612 | 14.526 | 1.00 | 33.76 |
| ATOM | 118 | CA | GLY | 29 | 19.182 | 20.477 | 15.170 | 1.00 | 33.68 |
| ATOM | 119 | C | GLY | 29 | 18.650 | 21.850 | 15.550 | 1.00 | 33.77 |
| ATOM | 120 | O | GLY | 29 | 19.382 | 22.844 | 15.513 | 1.00 | 34.35 |
| ATOM | 121 | N | PHE | 30 | 17.372 | 21.916 | 15.907 | 1.00 | 32.91 |
| ATOM | 122 | CA | PHE | 30 | 16.755 | 23.175 | 16.307 | 1.00 | 32.13 |
| ATOM | 123 | CB | PHE | 30 | 15.288 | 23.233 | 15.879 | 1.00 | 31.86 |
| ATOM | 124 | CG | PHE | 30 | 15.081 | 23.415 | 14.413 | 1.00 | 30.62 |
| ATOM | 125 | CD1 | PHE | 30 | 14.764 | 22.332 | 13.606 | 1.00 | 29.15 |
| ATOM | 126 | CD2 | PHE | 30 | 15.186 | 24.681 | 13.838 | 1.00 | 30.76 |
| ATOM | 127 | CE1 | PHE | 30 | 14.552 | 22.503 | 12.251 | 1.00 | 30.22 |
| ATOM | 128 | CE2 | PHE | 30 | 14.974 | 24.866 | 12.471 | 1.00 | 30.76 |
| ATOM | 129 | CZ | PHE | 30 | 14.656 | 23.777 | 11.676 | 1.00 | 30.15 |
| ATOM | 130 | C | PHE | 30 | 16.791 | 23.313 | 17.817 | 1.00 | 32.26 |
| ATOM | 131 | O | PHE | 30 | 16.502 | 22.361 | 18.540 | 1.00 | 32.31 |
| ATOM | 132 | N | PHE | 31 | 17.144 | 24.500 | 18.290 | 1.00 | 32.25 |
| ATOM | 133 | CA | PHE | 31 | 17.188 | 24.772 | 19.722 | 1.00 | 32.64 |
| ATOM | 134 | CB | PHE | 31 | 18.133 | 25.927 | 20.004 | 1.00 | 31.32 |
| ATOM | 135 | CG | PHE | 31 | 19.591 | 25.580 | 19.855 | 1.00 | 31.32 |
| ATOM | 136 | CD1 | PHE | 31 | 20.230 | 24.772 | 20.796 | 1.00 | 30.64 |
| ATOM | 137 | CD2 | PHE | 31 | 20.342 | 26.095 | 18.795 | 1.00 | 30.62 |
| ATOM | 138 | CE1 | PHE | 31 | 21.596 | 24.484 | 20.687 | 1.00 | 29.43 |
| ATOM | 139 | CE2 | PHE | 31 | 21.705 | 25.812 | 18.679 | 1.00 | 29.89 |
| ATOM | 140 | CZ | PHE | 31 | 22.332 | 25.006 | 19.629 | 1.00 | 29.14 |
| ATOM | 141 | C | PHE | 31 | 15.782 | 25.157 | 20.177 | 1.00 | 33.39 |
| ATOM | 142 | O | PHE | 31 | 15.086 | 25.897 | 19.479 | 1.00 | 34.48 |
| ATOM | 143 | N | LEU | 32 | 15.350 | 24.649 | 21.325 | 1.00 | 33.04 |
| ATOM | 144 | CA | LEU | 32 | 14.028 | 24.986 | 21.830 | 1.00 | 33.28 |
| ATOM | 145 | CB | LEU | 32 | 13.728 | 24.198 | 23.104 | 1.00 | 33.00 |
| ATOM | 146 | CG | LEU | 32 | 12.331 | 24.399 | 23.703 | 1.00 | 33.25 |
| ATOM | 147 | CD1 | LEU | 32 | 11.270 | 23.743 | 22.824 | 1.00 | 32.89 |
| ATOM | 148 | CD2 | LEU | 32 | 12.297 | 23.790 | 25.089 | 1.00 | 33.74 |
| ATOM | 149 | C | LEU | 32 | 14.028 | 26.481 | 22.138 | 1.00 | 33.64 |
| ATOM | 150 | O | LEU | 32 | 14.908 | 26.971 | 22.844 | 1.00 | 33.63 |
| ATOM | 151 | N | ARG | 33 | 13.045 | 27.204 | 21.609 | 1.00 | 34.43 |
| ATOM | 152 | CA | ARG | 33 | 12.969 | 28.646 | 21.827 | 1.00 | 34.97 |
| ATOM | 153 | CB | ARG | 33 | 13.186 | 29.389 | 20.513 | 1.00 | 33.91 |
| ATOM | 154 | CG | ARG | 33 | 13.249 | 30.890 | 20.669 | 1.00 | 33.39 |
| ATOM | 155 | CD | ARG | 33 | 13.680 | 31.570 | 19.369 | 1.00 | 33.21 |
| ATOM | 156 | NE | ARG | 33 | 12.734 | 31.334 | 18.281 | 1.00 | 33.57 |
| ATOM | 157 | CZ | ARG | 33 | 12.845 | 31.857 | 17.059 | 1.00 | 33.45 |
| ATOM | 158 | NH1 | ARG | 33 | 13.864 | 32.651 | 16.756 | 1.00 | 32.36 |
| ATOM | 159 | NH2 | ARG | 33 | 11.938 | 31.581 | 16.135 | 1.00 | 32.74 |
| ATOM | 160 | C | ARG | 33 | 11.672 | 29.128 | 22.460 | 1.00 | 35.77 |
| ATOM | 161 | O | ARG | 33 | 10.574 | 28.771 | 22.031 | 1.00 | 35.95 |
| ATOM | 162 | N | ILE | 34 | 11.817 | 29.948 | 23.490 | 1.00 | 36.85 |
| ATOM | 163 | CA | ILE | 34 | 10.678 | 30.507 | 24.195 | 1.00 | 38.11 |
| ATOM | 164 | CB | ILE | 34 | 10.791 | 30.237 | 25.715 | 1.00 | 37.62 |
| ATOM | 165 | CG2 | ILE | 34 | 9.704 | 30.988 | 26.461 | 1.00 | 36.83 |
| ATOM | 166 | CG1 | ILE | 34 | 10.698 | 28.730 | 25.979 | 1.00 | 36.70 |
| ATOM | 167 | CD1 | ILE | 34 | 10.892 | 28.345 | 27.430 | 1.00 | 37.36 |
| ATOM | 168 | C | ILE | 34 | 10.656 | 32.004 | 23.921 | 1.00 | 38.77 |
| ATOM | 169 | O | ILE | 34 | 11.515 | 32.738 | 24.397 | 1.00 | 38.44 |
| ATOM | 170 | N | HIS | 35 | 9.678 | 32.444 | 23.137 | 1.00 | 40.65 |
| ATOM | 171 | CA | HIS | 35 | 9.538 | 33.853 | 22.774 | 1.00 | 42.57 |
| ATOM | 172 | CB | HIS | 35 | 8.638 | 33.994 | 21.543 | 1.00 | 42.64 |
| ATOM | 173 | CG | HIS | 35 | 9.225 | 33.423 | 20.290 | 1.00 | 43.75 |
| ATOM | 174 | CD2 | HIS | 35 | 9.000 | 32.248 | 19.653 | 1.00 | 43.70 |
| ATOM | 175 | ND1 | HIS | 35 | 10.185 | 34.082 | 19.551 | 1.00 | 44.37 |
| ATOM | 176 | CE1 | HIS | 35 | 10.524 | 33.338 | 18.512 | 1.00 | 44.66 |
| ATOM | 177 | NE2 | HIS | 35 | 9.819 | 32.220 | 18.551 | 1.00 | 44.53 |
| ATOM | 178 | C | HIS | 35 | 8.939 | 34.681 | 23.902 | 1.00 | 43.65 |
| ATOM | 179 | O | HIS | 35 | 8.112 | 34.197 | 24.670 | 1.00 | 43.83 |
| ATOM | 180 | N | PRO | 36 | 9.347 | 35.952 | 24.009 | 1.00 | 45.01 |
| ATOM | 181 | CD | PRO | 36 | 10.350 | 36.645 | 23.180 | 1.00 | 45.93 |
| ATOM | 182 | CA | PRO | 36 | 8.832 | 36.842 | 25.052 | 1.00 | 45.83 |
| ATOM | 183 | CB | PRO | 36 | 9.462 | 38.184 | 24.700 | 1.00 | 45.55 |
| ATOM | 184 | CG | PRO | 36 | 10.755 | 37.792 | 24.073 | 1.00 | 46.12 |
| ATOM | 185 | C | PRO | 36 | 7.305 | 36.916 | 25.046 | 1.00 | 46.80 |
| ATOM | 186 | O | PRO | 36 | 6.689 | 37.107 | 26.091 | 1.00 | 47.50 |
| ATOM | 187 | N | ASP | 37 | 6.700 | 36.752 | 23.873 | 1.00 | 47.49 |
| ATOM | 188 | CA | ASP | 37 | 5.250 | 36.824 | 23.745 | 1.00 | 48.34 |
| ATOM | 189 | CB | ASP | 37 | 4.866 | 37.314 | 22.339 | 1.00 | 49.10 |
| ATOM | 190 | CG | ASP | 37 | 5.081 | 36.254 | 21.252 | 1.00 | 50.21 |
| ATOM | 191 | OD1 | ASP | 37 | 4.340 | 35.247 | 21.242 | 1.00 | 50.76 |
| ATOM | 192 | OD2 | ASP | 37 | 5.983 | 36.429 | 20.401 | 1.00 | 49.91 |
| ATOM | 193 | C | ASP | 37 | 4.524 | 35.515 | 24.044 | 1.00 | 48.65 |
| ATOM | 194 | O | ASP | 37 | 3.301 | 35.438 | 23.913 | 1.00 | 48.42 |
| ATOM | 195 | N | GLY | 38 | 5.266 | 34.485 | 24.438 | 1.00 | 48.59 |
| ATOM | 196 | CA | GLY | 38 | 4.631 | 33.213 | 24.744 | 1.00 | 48.28 |
| ATOM | 197 | C | GLY | 38 | 4.685 | 32.156 | 23.653 | 1.00 | 47.65 |
| ATOM | 198 | O | GLY | 38 | 4.202 | 31.043 | 23.842 | 1.00 | 47.29 |
| ATOM | 199 | N | ARG | 39 | 5.268 | 32.496 | 22.508 | 1.00 | 47.21 |
| ATOM | 200 | CA | ARG | 39 | 5.381 | 31.545 | 21.408 | 1.00 | 46.53 |
| ATOM | 201 | CB | ARG | 39 | 5.535 | 32.269 | 20.070 | 1.00 | 46.19 |
| ATOM | 202 | CG | ARG | 39 | 4.259 | 32.830 | 19.488 | 1.00 | 45.95 |
| ATOM | 203 | CD | ARG | 39 | 4.559 | 33.524 | 18.175 | 1.00 | 46.28 |
| ATOM | 204 | NE | ARG | 39 | 5.588 | 34.547 | 18.340 | 1.00 | 45.77 |
| ATOM | 205 | CZ | ARG | 39 | 6.647 | 34.674 | 17.547 | 1.00 | 46.38 |
| ATOM | 206 | NH1 | ARG | 39 | 7.533 | 35.636 | 17.780 | 1.00 | 46.10 |
| ATOM | 207 | NH2 | ARG | 39 | 6.822 | 33.836 | 16.524 | 1.00 | 46.14 |
| ATOM | 208 | C | ARG | 39 | 6.575 | 30.619 | 21.596 | 1.00 | 45.86 |
| ATOM | 209 | O | ARG | 39 | 7.654 | 31.060 | 21.991 | 1.00 | 46.25 |
| ATOM | 210 | N | VAL | 40 | 6.377 | 29.338 | 21.308 | 1.00 | 44.53 |
| ATOM | 211 | CA | VAL | 40 | 7.446 | 28.354 | 21.431 | 1.00 | 43.43 |
| ATOM | 212 | CB | VAL | 40 | 7.111 | 27.264 | 22.470 | 1.00 | 42.18 |
| ATOM | 213 | CG1 | VAL | 40 | 8.268 | 26.287 | 22.582 | 1.00 | 41.55 |
| ATOM | 214 | CG2 | VAL | 40 | 6.835 | 27.891 | 23.808 | 1.00 | 41.76 |
| ATOM | 215 | C | VAL | 40 | 7.713 | 27.660 | 20.100 | 1.00 | 43.16 |
| ATOM | 216 | O | VAL | 40 | 6.793 | 27.152 | 19.458 | 1.00 | 43.30 |
| ATOM | 217 | N | ASP | 41 | 8.973 | 27.644 | 19.687 | 1.00 | 42.42 |
| ATOM | 218 | CA | ASP | 41 | 9.364 | 26.986 | 18.446 | 1.00 | 41.97 |
| ATOM | 219 | CB | ASP | 41 | 9.053 | 27.875 | 17.240 | 1.00 | 40.91 |
| ATOM | 220 | CG | ASP | 41 | 9.874 | 29.148 | 17.219 | 1.00 | 41.31 |
| ATOM | 221 | OD1 | ASP | 41 | 9.666 | 29.969 | 16.304 | 1.00 | 41.93 |
| ATOM | 222 | OD2 | ASP | 41 | 10.732 | 29.336 | 18.108 | 1.00 | 41.72 |
| ATOM | 223 | C | ASP | 41 | 10.859 | 26.670 | 18.507 | 1.00 | 41.82 |
| ATOM | 224 | O | ASP | 41 | 11.461 | 26.691 | 19.583 | 1.00 | 41.15 |
| ATOM | 225 | N | GLY | 42 | 11.454 | 26.376 | 17.358 | 1.00 | 41.58 |
| ATOM | 226 | CA | GLY | 42 | 12.873 | 26.076 | 17.339 | 1.00 | 41.56 |
| ATOM | 227 | C | GLY | 42 | 13.650 | 26.897 | 16.324 | 1.00 | 41.64 |
| ATOM | 228 | O | GLY | 42 | 13.092 | 27.396 | 15.349 | 1.00 | 41.88 |
| ATOM | 229 | N | VAL | 43 | 14.943 | 27.059 | 16.574 | 1.00 | 41.45 |
| ATOM | 230 | CA | VAL | 43 | 15.819 | 27.780 | 15.666 | 1.00 | 41.09 |
| ATOM | 231 | CB | VAL | 43 | 15.923 | 29.266 | 16.002 | 1.00 | 41.11 |
| ATOM | 232 | CG1 | VAL | 43 | 14.600 | 29.927 | 15.702 | 1.00 | 41.85 |
| ATOM | 233 | CG2 | VAL | 43 | 16.320 | 29.460 | 17.456 | 1.00 | 40.06 |
| ATOM | 234 | C | VAL | 43 | 17.189 | 27.162 | 15.741 | 1.00 | 41.13 |
| ATOM | 235 | O | VAL | 43 | 17.559 | 26.585 | 16.756 | 1.00 | 40.93 |
| ATOM | 236 | N | ARG | 44 | 17.941 | 27.279 | 14.656 | 1.00 | 41.51 |
| ATOM | 237 | CA | ARG | 44 | 19.267 | 26.705 | 14.603 | 1.00 | 41.23 |
| ATOM | 238 | CB | ARG | 44 | 19.535 | 26.193 | 13.201 | 1.00 | 39.47 |
| ATOM | 239 | CG | ARG | 44 | 18.788 | 24.906 | 12.906 | 1.00 | 38.20 |
| ATOM | 240 | CD | ARG | 44 | 18.874 | 24.564 | 11.455 | 1.00 | 37.03 |
| ATOM | 241 | NE | ARG | 44 | 18.455 | 23.198 | 11.197 | 1.00 | 36.73 |
| ATOM | 242 | CZ | AEG | 44 | 17.801 | 22.821 | 10.104 | 1.00 | 36.87 |
| ATOM | 243 | NH1 | ARG | 44 | 17.486 | 23.716 | 9.174 | 1.00 | 36.61 |
| ATOM | 244 | NH2 | ARG | 44 | 17.477 | 21.549 | 9.930 | 1.00 | 35.75 |
| ATOM | 245 | C | ARG | 44 | 20.363 | 27.641 | 15.049 | 1.00 | 42.52 |
| ATOM | 246 | O | ARG | 44 | 21.406 | 27.190 | 15.501 | 1.00 | 43.57 |
| ATOM | 247 | N | GLU | 45 | 20.127 | 28.942 | 14.949 | 1.00 | 43.99 |
| ATOM | 248 | CA | GLU | 45 | 21.130 | 29.921 | 15.356 | 1.00 | 45.63 |
| ATOM | 249 | CB | GLU | 45 | 20.662 | 31.329 | 14.978 | 1.00 | 46.78 |
| ATOM | 250 | CG | GLU | 45 | 21.697 | 32.412 | 15.235 | 1.00 | 48.93 |
| ATOM | 251 | CD | GLU | 45 | 22.977 | 32.197 | 14.438 | 1.00 | 50.48 |
| ATOM | 252 | OE1 | GLU | 45 | 22.904 | 32.181 | 13.184 | 1.00 | 51.97 |
| ATOM | 253 | OE2 | GLU | 45 | 24.053 | 32.045 | 15.065 | 1.00 | 50.49 |
| ATOM | 254 | C | GLU | 45 | 21.421 | 29.856 | 16.856 | 1.00 | 45.57 |
| ATOM | 255 | O | GLU | 45 | 20.590 | 30.238 | 17.673 | 1.00 | 45.09 |
| ATOM | 256 | N | LYS | 46 | 22.614 | 29.379 | 17.201 | 1.00 | 46.06 |
| ATOM | 257 | CA | LYS | 46 | 23.030 | 29.247 | 18.592 | 1.00 | 46.74 |
| ATOM | 258 | CB | LYS | 46 | 24.396 | 28.553 | 18.660 | 1.00 | 46.79 |
| ATOM | 259 | CG | LYS | 46 | 25.061 | 28.592 | 20.038 | 1.00 | 47.95 |
| ATOM | 260 | CD | LYS | 46 | 25.708 | 27.261 | 20.403 | 1.00 | 48.10 |
| ATOM | 261 | CE | LYS | 46 | 26.700 | 27.403 | 21.553 | 1.00 | 48.89 |
| ATOM | 262 | NZ | LYS | 46 | 27.971 | 28.065 | 21.117 | 1.00 | 49.06 |
| ATOM | 263 | C | LYS | 46 | 23.077 | 30.565 | 19.367 | 1.00 | 47.09 |
| ATOM | 264 | O | LYS | 46 | 23.012 | 30.572 | 20.603 | 1.00 | 47.49 |
| ATOM | 265 | N | SER | 47 | 23.170 | 31.679 | 18.648 | 1.00 | 46.92 |
| ATOM | 266 | CA | SER | 47 | 23.242 | 32.990 | 19.285 | 1.00 | 46.51 |
| ATOM | 267 | CB | SER | 47 | 24.067 | 33.946 | 18.420 | 1.00 | 46.05 |
| ATOM | 268 | OG | SER | 47 | 23.487 | 34.109 | 17.137 | 1.00 | 46.10 |
| ATOM | 269 | C | SER | 47 | 21.887 | 33.626 | 19.596 | 1.00 | 46.68 |
| ATOM | 270 | O | SER | 47 | 21.831 | 34.697 | 20.204 | 1.00 | 46.55 |
| ATOM | 271 | N | ASP | 48 | 20.798 | 32.987 | 19.176 | 1.00 | 46.40 |
| ATOM | 272 | CA | ASP | 48 | 19.477 | 33.537 | 19.455 | 1.00 | 46.21 |
| ATOM | 273 | CB | ASP | 48 | 18.381 | 32.591 | 18.967 | 1.00 | 46.23 |
| ATOM | 274 | CG | ASP | 48 | 17.003 | 33.219 | 19.042 | 1.00 | 46.68 |
| ATOM | 275 | OD1 | ASP | 48 | 16.327 | 33.313 | 17.998 | 1.00 | 47.11 |
| ATOM | 276 | OD2 | ASP | 48 | 16.595 | 33.626 | 20.147 | 1.00 | 47.69 |
| ATOM | 277 | C | ASP | 48 | 19.374 | 33.736 | 20.968 | 1.00 | 46.01 |
| ATOM | 278 | O | ASP | 48 | 19.760 | 32.866 | 21.750 | 1.00 | 46.53 |
| ATOM | 279 | N | PRO | 49 | 18.857 | 34.891 | 21.403 | 1.00 | 45.50 |
| ATOM | 280 | CD | PRO | 49 | 18.476 | 36.072 | 20.608 | 1.00 | 45.51 |
| ATOM | 281 | CA | PRO | 49 | 18.731 | 35.162 | 22.838 | 1.00 | 45.10 |
| ATOM | 282 | CB | PRO | 49 | 18.564 | 36.682 | 22.879 | 1.00 | 45.08 |
| ATOM | 283 | CG | PRO | 49 | 17.772 | 36.942 | 21.629 | 1.00 | 45.21 |
| ATOM | 284 | C | PRO | 49 | 17.606 | 34.439 | 23.581 | 1.00 | 44.24 |
| ATOM | 285 | O | PRO | 49 | 17.645 | 34.319 | 24.807 | 1.00 | 44.38 |
| ATOM | 286 | N | HIS | 50 | 16.618 | 33.947 | 22.843 | 1.00 | 43.12 |
| ATOM | 287 | CA | HIS | 50 | 15.479 | 33.281 | 23.458 | 1.00 | 41.52 |
| ATOM | 288 | CB | HIS | 50 | 14.210 | 33.653 | 22.704 | 1.00 | 41.66 |
| ATOM | 289 | CG | HIS | 50 | 14.071 | 35.121 | 22.463 | 1.00 | 42.30 |
| ATOM | 290 | CD2 | HIS | 50 | 13.926 | 35.828 | 21.318 | 1.00 | 42.60 |
| ATOM | 291 | ND1 | HIS | 50 | 14.084 | 36.045 | 23.484 | 1.00 | 42.47 |
| ATOM | 292 | CE1 | HIS | 50 | 13.954 | 37.259 | 22.980 | 1.00 | 42.34 |
| ATOM | 293 | NE2 | HIS | 50 | 13.856 | 37.155 | 21.667 | 1.00 | 42.47 |
| ATOM | 294 | C | HIS | 50 | 15.564 | 31.771 | 23.570 | 1.00 | 40.32 |
| ATOM | 295 | O | HIS | 50 | 14.539 | 31.113 | 23.710 | 1.00 | 40.32 |
| ATOM | 296 | N | ILE | 51 | 16.769 | 31.215 | 23.505 | 1.00 | 39.52 |
| ATOM | 297 | CA | ILE | 51 | 16.923 | 29.766 | 23.630 | 1.00 | 38.51 |
| ATOM | 298 | CB | ILE | 51 | 17.654 | 29.138 | 22.411 | 1.00 | 38.26 |
| ATOM | 299 | CG2 | ILE | 51 | 16.797 | 29.306 | 21.156 | 1.00 | 36.50 |
| ATOM | 300 | CG1 | ILE | 51 | 19.056 | 29.744 | 22.261 | 1.00 | 38.02 |
| ATOM | 301 | CD1 | ILE | 51 | 19.892 | 29.091 | 21.186 | 1.00 | 37.88 |
| ATOM | 302 | C | ILE | 51 | 17.662 | 29.391 | 24.913 | 1.00 | 38.14 |
| ATOM | 303 | O | ILE | 51 | 17.821 | 28.215 | 25.223 | 1.00 | 37.89 |
| ATOM | 304 | N | LYS | 52 | 18.119 | 30.400 | 25.649 | 1.00 | 38.05 |
| ATOM | 305 | CA | LYS | 52 | 18.796 | 30.182 | 26.925 | 1.00 | 38.46 |
| ATOM | 306 | CB | LYS | 52 | 19.479 | 31.460 | 27.407 | 1.00 | 39.92 |
| ATOM | 307 | CG | LYS | 52 | 20.464 | 32.041 | 26.428 | 1.00 | 43.21 |
| ATOM | 308 | CD | LYS | 52 | 20.869 | 33.458 | 26.821 | 1.00 | 46.18 |
| ATOM | 309 | CE | LYS | 52 | 21.776 | 34.081 | 25.752 | 1.00 | 47.91 |
| ATOM | 310 | NZ | LYS | 52 | 22.998 | 33.244 | 25.518 | 1.00 | 48.79 |
| ATOM | 311 | C | LYS | 52 | 17.677 | 29.838 | 27.896 | 1.00 | 37.80 |
| ATOM | 312 | O | LYS | 52 | 16.835 | 30.686 | 28.214 | 1.00 | 37.80 |
| ATOM | 313 | N | LEU | 53 | 17.666 | 28.599 | 28.370 | 1.00 | 36.17 |
| ATOM | 314 | CA | LEU | 53 | 16.620 | 28.150 | 29.266 | 1.00 | 33.73 |
| ATOM | 315 | CB | LEU | 53 | 15.942 | 26.928 | 28.648 | 1.00 | 32.80 |
| ATOM | 316 | CG | LEU | 53 | 15.591 | 27.119 | 27.168 | 1.00 | 31.74 |
| ATOM | 317 | CD1 | LEU | 53 | 15.106 | 25.828 | 26.547 | 1.00 | 31.06 |
| ATOM | 318 | CD2 | LEU | 53 | 14.528 | 28.182 | 27.058 | 1.00 | 31.89 |
| ATOM | 319 | C | LEU | 53 | 17.147 | 27.817 | 30.647 | 1.00 | 33.17 |
| ATOM | 320 | O | LEU | 53 | 18.310 | 27.487 | 30.822 | 1.00 | 32.86 |
| ATOM | 321 | N | GLN | 54 | 16.274 | 27.914 | 31.634 | 1.00 | 32.71 |
| ATOM | 322 | CA | GLN | 54 | 16.652 | 27.605 | 32.995 | 1.00 | 32.58 |
| ATOM | 323 | CB | GLN | 54 | 16.363 | 28.802 | 33.896 | 1.00 | 32.34 |
| ATOM | 324 | CG | GLN | 54 | 17.001 | 28.705 | 35.249 | 1.00 | 31.47 |
| ATOM | 325 | CD | GLN | 54 | 18.497 | 28.699 | 35.143 | 1.00 | 31.55 |
| ATOM | 326 | OE1 | GLN | 54 | 19.068 | 29.545 | 34.465 | 1.00 | 32.74 |
| ATOM | 327 | NE2 | GLN | 54 | 19.148 | 27.750 | 35.811 | 1.00 | 31.36 |
| ATOM | 328 | C | GLN | 54 | 15.827 | 26.400 | 33.432 | 1.00 | 32.20 |
| ATOM | 329 | O | GLN | 54 | 14.624 | 26.511 | 33.648 | 1.00 | 33.12 |
| ATOM | 330 | N | LEU | 55 | 16.478 | 25.249 | 33.541 | 1.00 | 31.21 |
| ATOM | 331 | CA | LEU | 55 | 15.816 | 24.025 | 33.939 | 1.00 | 30.46 |
| ATOM | 332 | CB | LEU | 55 | 16.482 | 22.845 | 33.232 | 1.00 | 29.98 |
| ATOM | 333 | CG | LEU | 55 | 16.557 | 23.052 | 31.714 | 1.00 | 29.60 |
| ATOM | 334 | CD1 | LEU | 55 | 17.358 | 21.971 | 31.048 | 1.00 | 29.73 |
| ATOM | 335 | CD2 | LEU | 55 | 15.159 | 23.054 | 31.162 | 1.00 | 29.86 |
| ATOM | 336 | C | LEU | 55 | 15.933 | 23.917 | 35.450 | 1.00 | 30.59 |
| ATOM | 337 | O | LEU | 55 | 17.026 | 23.876 | 36.004 | 1.00 | 31.00 |
| ATOM | 338 | N | GLN | 56 | 14.786 | 23.879 | 36.114 | 1.00 | 31.09 |
| ATOM | 339 | CA | GLN | 56 | 14.727 | 23.826 | 37.565 | 1.00 | 30.72 |
| ATOM | 340 | CB | GLN | 56 | 14.075 | 25.125 | 38.060 | 1.00 | 29.53 |
| ATOM | 341 | CG | GLN | 56 | 13.885 | 25.231 | 39.551 | 1.00 | 29.08 |
| ATOM | 342 | CD | GLN | 56 | 15.195 | 25.215 | 40.319 | 1.00 | 29.28 |
| ATOM | 343 | OE1 | GLN | 56 | 16.022 | 26.117 | 40.180 | 1.00 | 27.65 |
| ATOM | 344 | NE2 | GLN | 56 | 15.383 | 24.185 | 41.147 | 1.00 | 29.56 |
| ATOM | 345 | C | GLN | 56 | 13.938 | 22.610 | 38.049 | 1.00 | 30.75 |
| ATOM | 346 | O | GLN | 56 | 12.785 | 22.419 | 37.677 | 1.00 | 31.32 |
| ATOM | 347 | N | ALA | 57 | 14.563 | 21.788 | 38.880 | 1.00 | 30.82 |
| ATOM | 348 | CA | ALA | 57 | 13.891 | 20.612 | 39.407 | 1.00 | 31.62 |
| ATOM | 349 | CB | ALA | 57 | 14.905 | 19.669 | 40.031 | 1.00 | 29.49 |
| ATOM | 350 | C | ALA | 57 | 12.893 | 21.071 | 40.459 | 1.00 | 32.63 |
| ATOM | 351 | O | ALA | 57 | 13.217 | 21.929 | 41.285 | 1.00 | 32.89 |
| ATOM | 352 | N | GLU | 58 | 11.685 | 20.515 | 40.420 | 1.00 | 32.95 |
| ATOM | 353 | CA | GLU | 58 | 10.647 | 20.851 | 41.387 | 1.00 | 33.67 |
| ATOM | 354 | CB | GLU | 58 | 9.290 | 20.862 | 40.702 | 1.00 | 33.94 |
| ATOM | 355 | CG | GLU | 58 | 8.277 | 21.746 | 41.379 | 1.00 | 34.98 |
| ATOM | 356 | CD | GLU | 58 | 8.813 | 23.140 | 41.604 | 1.00 | 35.72 |
| ATOM | 357 | OE1 | GLU | 58 | 9.533 | 23.653 | 40.716 | 1.00 | 37.44 |
| ATOM | 358 | OE2 | GLU | 58 | 8.509 | 23.729 | 42.658 | 1.00 | 36.47 |
| ATOM | 359 | C | GLU | 58 | 10.700 | 19.745 | 42.434 | 1.00 | 34.07 |
| ATOM | 360 | O | GLU | 58 | 10.379 | 19.938 | 43.605 | 1.00 | 33.94 |
| ATOM | 361 | N | GLU | 59 | 11.105 | 18.572 | 41.971 | 1.00 | 34.50 |
| ATOM | 362 | CA | GLU | 59 | 11.283 | 17.398 | 42.807 | 1.00 | 34.71 |
| ATOM | 363 | CB | GLU | 59 | 9.948 | 16.806 | 43.244 | 1.00 | 34.51 |
| ATOM | 364 | CG | GLU | 59 | 9.123 | 16.202 | 42.170 | 1.00 | 35.87 |
| ATOM | 365 | CD | GLU | 59 | 7.769 | 15.816 | 42.707 | 1.00 | 37.28 |
| ATOM | 366 | OE1 | GLU | 59 | 6.988 | 16.742 | 43.031 | 1.00 | 37.78 |
| ATOM | 367 | OE2 | GLU | 59 | 7.495 | 14.598 | 42.825 | 1.00 | 37.92 |
| ATOM | 368 | C | GLU | 59 | 12.083 | 16.420 | 41.971 | 1.00 | 34.27 |
| ATOM | 369 | O | GLU | 59 | 12.424 | 16.727 | 40.834 | 1.00 | 34.69 |
| ATOM | 370 | N | ARG | 60 | 12.405 | 15.257 | 42.522 | 1.00 | 34.11 |
| ATOM | 371 | CA | ARG | 60 | 13.198 | 14.284 | 41.782 | 1.00 | 33.43 |
| ATOM | 372 | CB | ARG | 60 | 13.335 | 12.975 | 42.561 | 1.00 | 34.80 |
| ATOM | 373 | CG | ARG | 60 | 14.590 | 12.869 | 43.384 | 1.00 | 37.91 |
| ATOM | 374 | CD | ARG | 60 | 14.742 | 11.464 | 43.954 | 1.00 | 40.21 |
| ATOM | 375 | NE | ARG | 60 | 14.480 | 10.470 | 42.918 | 1.00 | 44.07 |
| ATOM | 376 | CZ | ARG | 60 | 14.911 | 9.208 | 42.934 | 1.00 | 45.24 |
| ATOM | 377 | NH1 | ARG | 60 | 15.643 | 8.757 | 43.942 | 1.00 | 44.84 |
| ATOM | 378 | NH2 | ARG | 60 | 14.610 | 8.396 | 41.924 | 1.00 | 45.82 |
| ATOM | 379 | C | ARG | 60 | 12.685 | 13.964 | 40.388 | 1.00 | 31.80 |
| ATOM | 380 | O | ARG | 60 | 11.559 | 13.502 | 40.220 | 1.00 | 31.15 |
| ATOM | 381 | N | GLY | 61 | 13.531 | 14.218 | 39.395 | 1.00 | 30.60 |
| ATOM | 382 | CA | GLY | 61 | 13.200 | 13.916 | 38.013 | 1.00 | 29.48 |
| ATOM | 383 | C | GLY | 61 | 12.147 | 14.778 | 37.351 | 1.00 | 28.97 |
| ATOM | 384 | O | GLY | 61 | 11.782 | 14.540 | 36.199 | 1.00 | 28.95 |
| ATOM | 385 | N | VAL | 62 | 11.656 | 15.780 | 38.074 | 1.00 | 28.54 |
| ATOM | 386 | CA | VAL | 62 | 10.627 | 16.679 | 37.554 | 1.00 | 27.15 |
| ATOM | 387 | CB | VAL | 62 | 9.395 | 16.718 | 38.487 | 1.00 | 25.88 |
| ATOM | 388 | CG1 | VAL | 62 | 8.448 | 17.817 | 38.053 | 1.00 | 22.76 |
| ATOM | 389 | CG2 | VAL | 62 | 8.680 | 15.364 | 38.469 | 1.00 | 25.15 |
| ATOM | 390 | C | VAL | 62 | 11.179 | 18.088 | 37.456 | 1.00 | 27.19 |
| ATOM | 391 | O | VAL | 62 | 11.647 | 18.636 | 38.448 | 1.00 | 27.64 |
| ATOM | 392 | N | VAL | 63 | 11.116 | 18.683 | 36.270 | 1.00 | 26.77 |
| ATOM | 393 | CA | VAL | 63 | 11.619 | 20.040 | 36.095 | 1.00 | 26.70 |
| ATOM | 394 | CB | VAL | 63 | 12.911 | 20.057 | 35.236 | 1.00 | 26.17 |
| ATOM | 395 | CG1 | VAL | 63 | 13.946 | 19.123 | 35.822 | 1.00 | 25.10 |
| ATOM | 396 | CG2 | VAL | 63 | 12.588 | 19.656 | 33.812 | 1.00 | 25.90 |
| ATOM | 397 | C | VAL | 63 | 10.631 | 20.996 | 35.423 | 1.00 | 27.27 |
| ATOM | 398 | O | VAL | 63 | 9.608 | 20.583 | 34.872 | 1.00 | 27.24 |
| ATOM | 399 | N | SER | 64 | 10.958 | 22.281 | 35.497 | 1.00 | 27.10 |
| ATOM | 400 | CA | SER | 64 | 10.184 | 23.325 | 34.856 | 1.00 | 27.15 |
| ATOM | 401 | CB | SER | 64 | 9.714 | 24.383 | 35.860 | 1.00 | 27.28 |
| ATOM | 402 | OG | SER | 64 | 10.732 | 25.312 | 36.206 | 1.00 | 27.39 |
| ATOM | 403 | C | SER | 64 | 11.205 | 23.919 | 33.889 | 1.00 | 27.74 |
| ATOM | 404 | O | SER | 64 | 12.408 | 23.908 | 34.156 | 1.00 | 28.32 |
| ATOM | 405 | N | ILE | 65 | 10.738 | 24.427 | 32.764 | 1.00 | 27.80 |
| ATOM | 406 | CA | ILE | 65 | 11.633 | 24.982 | 31.769 | 1.00 | 28.35 |
| ATOM | 407 | CB | ILE | 65 | 11.512 | 24.168 | 30.468 | 1.00 | 27.17 |
| ATOM | 408 | CG2 | ILE | 65 | 12.444 | 24.709 | 29.419 | 1.00 | 26.25 |
| ATOM | 409 | CG1 | ILE | 65 | 11.812 | 22.695 | 30.773 | 1.00 | 27.35 |
| ATOM | 410 | CD1 | ILE | 65 | 11.570 | 21.747 | 29.611 | 1.00 | 27.91 |
| ATOM | 411 | C | ILE | 65 | 11.282 | 26.446 | 31.538 | 1.00 | 29.42 |
| ATOM | 412 | O | ILE | 65 | 10.243 | 26.767 | 30.968 | 1.00 | 29.87 |
| ATOM | 413 | N | LYS | 66 | 12.159 | 27.330 | 31.985 | 1.00 | 30.13 |
| ATOM | 414 | CA | LYS | 66 | 11.925 | 28.755 | 31.861 | 1.00 | 31.66 |
| ATOM | 415 | CB | LYS | 66 | 12.102 | 29.407 | 33.234 | 1.00 | 32.15 |
| ATOM | 416 | CG | LYS | 66 | 11.817 | 30.878 | 33.255 | 1.00 | 33.67 |
| ATOM | 417 | CD | LYS | 66 | 12.204 | 31.476 | 34.583 | 1.00 | 34.82 |
| ATOM | 418 | CE | LYS | 66 | 11.748 | 32.922 | 34.672 | 1.00 | 36.66 |
| ATOM | 419 | NZ | LYS | 66 | 12.031 | 33.530 | 36.011 | 1.00 | 38.73 |
| ATOM | 420 | C | LYS | 66 | 12.822 | 29.454 | 30.848 | 1.00 | 31.95 |
| ATOM | 421 | O | LYS | 66 | 14.043 | 29.331 | 30.905 | 1.00 | 32.37 |
| ATOM | 422 | N | GLY | 67 | 12.210 | 30.188 | 29.926 | 1.00 | 32.75 |
| ATOM | 423 | CA | GLY | 67 | 12.979 | 30.926 | 28.941 | 1.00 | 34.20 |
| ATOM | 424 | C | GLY | 67 | 13.478 | 32.164 | 29.656 | 1.00 | 35.22 |
| ATOM | 425 | O | GLY | 67 | 12.688 | 33.018 | 30.037 | 1.00 | 36.31 |
| ATOM | 426 | N | VAL | 68 | 14.785 | 32.260 | 29.850 | 1.00 | 35.63 |
| ATOM | 427 | CA | VAL | 68 | 15.375 | 33.383 | 30.561 | 1.00 | 37.07 |
| ATOM | 428 | CB | VAL | 68 | 16.900 | 33.314 | 30.483 | 1.00 | 36.39 |
| ATOM | 429 | CG1 | VAL | 68 | 17.509 | 34.445 | 31.278 | 1.00 | 35.24 |
| ATOM | 430 | CG2 | VAL | 68 | 17.371 | 31.969 | 31.010 | 1.00 | 36.82 |
| ATOM | 431 | C | VAL | 68 | 14.928 | 34.780 | 30.121 | 1.00 | 38.09 |
| ATOM | 432 | O | VAL | 68 | 14.363 | 35.537 | 30.912 | 1.00 | 38.43 |
| ATOM | 433 | N | SER | 69 | 15.179 | 35.133 | 28.870 | 1.00 | 38.89 |
| ATOM | 434 | CA | SER | 69 | 14.787 | 36.454 | 28.412 | 1.00 | 39.28 |
| ATOM | 435 | CB | SER | 69 | 15.455 | 36.780 | 27.080 | 1.00 | 38.34 |
| ATOM | 436 | OG | SER | 69 | 14.629 | 36.377 | 26.013 | 1.00 | 39.34 |
| ATOM | 437 | C | SER | 69 | 13.270 | 36.616 | 28.293 | 1.00 | 39.46 |
| ATOM | 438 | O | SER | 69 | 12.751 | 37.704 | 28.518 | 1.00 | 40.48 |
| ATOM | 439 | N | ALA | 70 | 12.555 | 35.551 | 27.952 | 1.00 | 39.48 |
| ATOM | 440 | CA | ALA | 70 | 11.102 | 35.645 | 27.826 | 1.00 | 39.59 |
| ATOM | 441 | CB | ALA | 70 | 10.565 | 34.441 | 27.064 | 1.00 | 39.12 |
| ATOM | 442 | C | ALA | 70 | 10.436 | 35.724 | 29.190 | 1.00 | 39.70 |
| ATOM | 443 | O | ALA | 70 | 9.306 | 36.191 | 29.320 | 1.00 | 40.04 |
| ATOM | 444 | N | ASN | 71 | 11.144 | 35.254 | 30.208 | 1.00 | 39.82 |
| ATOM | 445 | CA | ASN | 71 | 10.633 | 35.246 | 31.567 | 1.00 | 39.73 |
| ATOM | 446 | CB | ASN | 71 | 10.442 | 36.683 | 32.077 | 1.00 | 39.99 |
| ATOM | 447 | CG | ASN | 71 | 10.387 | 36.761 | 33.603 | 1.00 | 40.30 |
| ATOM | 448 | OD1 | ASN | 71 | 11.195 | 36.140 | 34.287 | 1.00 | 40.54 |
| ATOM | 449 | ND2 | ASN | 71 | 9.441 | 37.531 | 34.135 | 1.00 | 39.59 |
| ATOM | 450 | C | ASN | 71 | 9.314 | 34.477 | 31.629 | 1.00 | 39.75 |
| ATOM | 451 | O | ASN | 71 | 8.403 | 34.835 | 32.379 | 1.00 | 40.53 |
| ATOM | 452 | N | ARG | 72 | 9.217 | 33.416 | 30.834 | 1.00 | 38.92 |
| ATOM | 453 | CA | ARG | 72 | 8.022 | 32.580 | 30.807 | 1.00 | 38.05 |
| ATOM | 454 | CB | ARG | 72 | 7.269 | 32.768 | 29.495 | 1.00 | 37.29 |
| ATOM | 455 | CG | ARG | 72 | 6.533 | 34.076 | 29.361 | 1.00 | 37.12 |
| ATOM | 456 | CD | ARG | 72 | 6.058 | 34.238 | 27.921 | 1.00 | 37.64 |
| ATOM | 457 | NE | ARG | 72 | 5.254 | 35.439 | 27.721 | 1.00 | 37.13 |
| ATOM | 458 | CZ | ARG | 72 | 3.935 | 35.495 | 27.863 | 1.00 | 36.23 |
| ATOM | 459 | NH1 | ARG | 72 | 3.245 | 34.419 | 28.201 | 1.00 | 35.39 |
| ATOM | 460 | NH2 | ARG | 72 | 3.308 | 36.641 | 27.674 | 1.00 | 36.87 |
| ATOM | 461 | C | ARG | 72 | 8.395 | 31.105 | 30.958 | 1.00 | 37.94 |
| ATOM | 462 | O | ARG | 72 | 9.508 | 30.697 | 30.625 | 1.00 | 37.86 |
| ATOM | 463 | N | TYR | 73 | 7.451 | 30.313 | 31.455 | 1.00 | 37.74 |
| ATOM | 464 | CA | TYR | 73 | 7.652 | 28.883 | 31.655 | 1.00 | 36.75 |
| ATOM | 465 | CB | TYR | 73 | 7.085 | 28.449 | 33.002 | 1.00 | 36.28 |
| ATOM | 466 | CG | TYR | 73 | 7.695 | 29.181 | 34.149 | 1.00 | 35.96 |
| ATOM | 467 | CD1 | TYR | 73 | 7.225 | 30.438 | 34.529 | 1.00 | 36.06 |
| ATOM | 468 | CE1 | TYR | 73 | 7.835 | 31.148 | 35.554 | 1.00 | 35.44 |
| ATOM | 469 | CD2 | TYR | 73 | 8.787 | 28.650 | 34.823 | 1.00 | 35.68 |
| ATOM | 470 | CE2 | TYR | 73 | 9.407 | 29.349 | 35.843 | 1.00 | 36.31 |
| ATOM | 471 | CZ | TYR | 73 | 8.928 | 30.596 | 36.204 | 1.00 | 36.67 |
| ATOM | 472 | OH | TYR | 73 | 9.564 | 31.281 | 37.209 | 1.00 | 38.11 |
| ATOM | 473 | C | TYR | 73 | 6.972 | 28.067 | 30.572 | 1.00 | 36.72 |
| ATOM | 474 | O | TYR | 73 | 5.833 | 28.337 | 30.198 | 1.00 | 37.00 |
| ATOM | 475 | N | LEU | 74 | 7.666 | 27.054 | 30.080 | 1.00 | 36.29 |
| ATOM | 476 | CA | LEU | 74 | 7.104 | 26.201 | 29.055 | 1.00 | 35.89 |
| ATOM | 477 | CB | LEU | 74 | 8.177 | 25.246 | 28.536 | 1.00 | 34.95 |
| ATOM | 478 | CG | LEU | 74 | 7.730 | 24.233 | 27.488 | 1.00 | 33.69 |
| ATOM | 479 | CD1 | LEU | 74 | 7.607 | 24.944 | 26.150 | 1.00 | 34.02 |
| ATOM | 480 | CD2 | LEU | 74 | 8.731 | 23.095 | 27.408 | 1.00 | 33.30 |
| ATOM | 481 | C | LEU | 74 | 5.949 | 25.406 | 29.652 | 1.00 | 36.28 |
| ATOM | 482 | O | LEU | 74 | 6.042 | 24.907 | 30.776 | 1.00 | 35.45 |
| ATOM | 483 | N | ALA | 75 | 4.862 | 25.292 | 28.895 | 1.00 | 37.11 |
| ATOM | 484 | CA | ALA | 75 | 3.691 | 24.545 | 29.344 | 1.00 | 38.41 |
| ATOM | 485 | CB | ALA | 75 | 2.660 | 25.491 | 29.960 | 1.00 | 37.93 |
| ATOM | 486 | C | ALA | 75 | 3.068 | 23.818 | 28.170 | 1.00 | 39.32 |
| ATOM | 487 | O | ALA | 75 | 3.084 | 24.323 | 27.048 | 1.00 | 39.85 |
| ATOM | 488 | N | MET | 76 | 2.530 | 22.629 | 28.427 | 1.00 | 40.20 |
| ATOM | 489 | CA | MET | 76 | 1.860 | 21.856 | 27.386 | 1.00 | 41.55 |
| ATOM | 490 | CB | MET | 76 | 2.420 | 20.438 | 27.279 | 1.00 | 41.81 |
| ATOM | 491 | CG | MET | 76 | 1.754 | 19.646 | 26.172 | 1.00 | 41.84 |
| ATOM | 492 | SD | MET | 76 | 2.515 | 18.069 | 25.840 | 1.00 | 45.00 |
| ATOM | 493 | CE | MET | 76 | 1.593 | 17.044 | 26.896 | 1.00 | 44.29 |
| ATOM | 494 | C | MET | 76 | 0.382 | 21.786 | 27.743 | 1.00 | 42.49 |
| ATOM | 495 | O | MET | 76 | 0.024 | 21.447 | 28.872 | 1.00 | 41.69 |
| ATOM | 496 | N | LYS | 77 | −0.475 | 22.093 | 26.775 | 1.00 | 43.54 |
| ATOM | 497 | CA | LYS | 77 | −1.906 | 22.106 | 27.019 | 1.00 | 44.58 |
| ATOM | 498 | CB | LYS | 77 | −2.553 | 23.140 | 26.113 | 1.00 | 44.50 |
| ATOM | 499 | CG | LYS | 77 | −1.814 | 24.457 | 26.102 | 1.00 | 45.41 |
| ATOM | 500 | CD | LYS | 77 | −2.451 | 25.481 | 27.027 | 1.00 | 46.70 |
| ATOM | 501 | CE | LYS | 77 | −2.364 | 25.068 | 28.474 | 1.00 | 46.80 |
| ATOM | 502 | NZ | LYS | 77 | −2.880 | 26.148 | 29.356 | 1.00 | 47.00 |
| ATOM | 503 | C | LYS | 77 | −2.585 | 20.755 | 26.842 | 1.00 | 44.85 |
| ATOM | 504 | O | LYS | 77 | −1.953 | 19.778 | 26.443 | 1.00 | 44.74 |
| ATOM | 505 | N | GLU | 78 | −3.880 | 20.728 | 27.146 | 1.00 | 45.05 |
| ATOM | 506 | CA | GLU | 78 | −4.711 | 19.537 | 27.057 | 1.00 | 45.21 |
| ATOM | 507 | CB | GLU | 78 | −6.124 | 19.865 | 27.531 | 1.00 | 44.54 |
| ATOM | 508 | CG | GLU | 78 | −6.904 | 20.817 | 26.625 | 1.00 | 44.11 |
| ATOM | 509 | CD | GLU | 78 | −6.328 | 22.231 | 26.562 | 1.00 | 44.45 |
| ATOM | 510 | OE1 | GLU | 78 | −5.909 | 22.770 | 27.615 | 1.00 | 43.37 |
| ATOM | 511 | OE2 | GLU | 78 | −6.316 | 22.815 | 25.453 | 1.00 | 44.41 |
| ATOM | 512 | C | GLU | 78 | −4.787 | 18.964 | 25.647 | 1.00 | 45.90 |
| ATOM | 513 | O | GLU | 78 | −4.994 | 17.760 | 25.465 | 1.00 | 46.07 |
| ATOM | 514 | N | ASP | 79 | −4.642 | 19.828 | 24.647 | 1.00 | 45.95 |
| ATOM | 515 | CA | ASP | 79 | −4.695 | 19.382 | 23.256 | 1.00 | 45.54 |
| ATOM | 516 | CB | ASP | 79 | −5.215 | 20.495 | 22.342 | 1.00 | 45.86 |
| ATOM | 517 | CG | ASP | 79 | −4.272 | 21.680 | 22.279 | 1.00 | 47.04 |
| ATOM | 518 | OD1 | ASP | 79 | −4.444 | 22.551 | 21.398 | 1.00 | 47.93 |
| ATOM | 519 | OD2 | ASP | 79 | −3.354 | 21.748 | 23.120 | 1.00 | 47.75 |
| ATOM | 520 | C | ASP | 79 | −3.317 | 18.956 | 22.771 | 1.00 | 44.86 |
| ATOM | 521 | O | ASP | 79 | −3.184 | 18.346 | 21.711 | 1.00 | 45.40 |
| ATOM | 522 | N | GLY | 80 | −2.291 | 19.288 | 23.543 | 1.00 | 43.77 |
| ATOM | 523 | CA | GLY | 80 | −0.942 | 18.922 | 23.166 | 1.00 | 42.64 |
| ATOM | 524 | C | GLY | 80 | −0.095 | 20.066 | 22.639 | 1.00 | 42.17 |
| ATOM | 525 | O | GLY | 80 | 1.094 | 19.885 | 22.374 | 1.00 | 41.86 |
| ATOM | 526 | N | ARG | 81 | −0.683 | 21.248 | 22.483 | 1.00 | 41.13 |
| ATOM | 527 | CA | ARG | 81 | 0.083 | 22.373 | 21.970 | 1.00 | 40.34 |
| ATOM | 528 | CB | ARG | 81 | −0.845 | 23.457 | 21.409 | 1.00 | 39.97 |
| ATOM | 529 | CG | ARG | 81 | −1.616 | 24.242 | 22.436 | 1.00 | 39.13 |
| ATOM | 530 | CD | ARG | 81 | −2.404 | 25.358 | 21.781 | 1.00 | 38.12 |
| ATOM | 531 | NE | ARG | 81 | −3.042 | 26.185 | 22.795 | 1.00 | 38.02 |
| ATOM | 532 | CZ | ARG | 81 | −3.965 | 25.734 | 23.639 | 1.00 | 38.95 |
| ATOM | 533 | NH1 | ARG | 81 | −4.361 | 24.464 | 23.580 | 1.00 | 38.84 |
| ATOM | 534 | NH2 | ARG | 81 | −4.481 | 26.546 | 24.553 | 1.00 | 38.18 |
| ATOM | 535 | C | ARG | 81 | 0.995 | 22.950 | 23.046 | 1.00 | 39.91 |
| ATOM | 536 | O | ARG | 81 | 0.751 | 22.765 | 24.242 | 1.00 | 40.38 |
| ATOM | 537 | N | LEU | 82 | 2.056 | 23.631 | 22.616 | 1.00 | 38.75 |
| ATOM | 538 | CA | LEU | 82 | 3.022 | 24.222 | 23.540 | 1.00 | 37.93 |
| ATOM | 539 | CB | LEU | 82 | 4.456 | 23.802 | 23.171 | 1.00 | 36.21 |
| ATOM | 540 | CG | LEU | 82 | 4.829 | 22.315 | 23.052 | 1.00 | 34.01 |
| ATOM | 541 | CD1 | LEU | 82 | 6.329 | 22.173 | 22.841 | 1.00 | 32.17 |
| ATOM | 542 | CD2 | LEU | 82 | 4.406 | 21.582 | 24.304 | 1.00 | 32.98 |
| ATOM | 543 | C | LEU | 82 | 2.962 | 25.740 | 23.566 | 1.00 | 38.10 |
| ATOM | 544 | O | LEU | 82 | 2.668 | 26.383 | 22.559 | 1.00 | 38.60 |
| ATOM | 545 | N | LEU | 83 | 3.246 | 26.314 | 24.724 | 1.00 | 38.19 |
| ATOM | 546 | CA | LEU | 83 | 3.254 | 27.763 | 24.862 | 1.00 | 38.46 |
| ATOM | 547 | CB | LEU | 83 | 1.826 | 28.314 | 24.901 | 1.00 | 37.52 |
| ATOM | 548 | CG | LEU | 83 | 0.862 | 27.819 | 25.981 | 1.00 | 37.54 |
| ATOM | 549 | CD1 | LEU | 83 | 1.342 | 28.260 | 27.360 | 1.00 | 36.95 |
| ATOM | 550 | CD2 | LEU | 83 | −0.537 | 28.369 | 25.696 | 1.00 | 36.58 |
| ATOM | 551 | C | LEU | 83 | 4.009 | 28.118 | 26.129 | 1.00 | 38.76 |
| ATOM | 552 | O | LEU | 83 | 4.258 | 27.252 | 26.967 | 1.00 | 39.02 |
| ATOM | 553 | N | ALA | 84 | 4.385 | 29.383 | 26.265 | 1.00 | 39.18 |
| ATOM | 554 | CA | ALA | 84 | 5.120 | 29.813 | 27.445 | 1.00 | 40.26 |
| ATOM | 555 | CB | ALA | 84 | 6.376 | 30.565 | 27.037 | 1.00 | 40.39 |
| ATOM | 556 | C | ALA | 84 | 4.256 | 30.682 | 28.347 | 1.00 | 40.54 |
| ATOM | 557 | O | ALA | 84 | 3.981 | 31.837 | 28.034 | 1.00 | 40.58 |
| ATOM | 558 | N | SER | 85 | 3.832 | 30.122 | 29.477 | 1.00 | 40.83 |
| ATOM | 559 | CA | SER | 85 | 2.995 | 30.872 | 30.393 | 1.00 | 40.42 |
| ATOM | 560 | CB | SER | 85 | 2.084 | 29.944 | 31.207 | 1.00 | 39.27 |
| ATOM | 561 | OG | SER | 85 | 2.701 | 29.482 | 32.387 | 1.00 | 39.17 |
| ATOM | 562 | C | SER | 85 | 3.824 | 31.763 | 31.302 | 1.00 | 41.15 |
| ATOM | 563 | O | SER | 85 | 4.985 | 31.478 | 31.598 | 1.00 | 41.03 |
| ATOM | 564 | N | LYS | 86 | 3.219 | 32.867 | 31.721 | 1.00 | 41.83 |
| ATOM | 565 | CA | LYS | 86 | 3.889 | 33.831 | 32.582 | 1.00 | 42.61 |
| ATOM | 566 | CB | LYS | 86 | 3.161 | 35.179 | 32.549 | 1.00 | 42.91 |
| ATOM | 567 | CG | LYS | 86 | 4.079 | 36.405 | 32.586 | 1.00 | 43.72 |
| ATOM | 568 | CD | LYS | 86 | 4.949 | 36.496 | 31.320 | 1.00 | 44.37 |
| ATOM | 569 | CE | LYS | 86 | 5.702 | 37.838 | 31.197 | 1.00 | 43.85 |
| ATOM | 570 | NZ | LYS | 86 | 6.653 | 38.119 | 32.319 | 1.00 | 43.40 |
| ATOM | 571 | C | LYS | 86 | 3.958 | 33.326 | 34.012 | 1.00 | 42.58 |
| ATOM | 572 | O | LYS | 86 | 4.888 | 33.664 | 34.742 | 1.00 | 43.32 |
| ATOM | 573 | N | SER | 87 | 2.978 | 32.529 | 34.423 | 1.00 | 42.41 |
| ATOM | 574 | CA | SER | 87 | 2.990 | 31.985 | 35.780 | 1.00 | 42.99 |
| ATOM | 575 | CB | SER | 87 | 1.769 | 32.459 | 36.578 | 1.00 | 43.28 |
| ATOM | 576 | OG | SER | 87 | 0.566 | 32.014 | 35.988 | 1.00 | 45.27 |
| ATOM | 577 | C | SER | 87 | 3.054 | 30.459 | 35.757 | 1.00 | 42.54 |
| ATOM | 578 | O | SER | 87 | 2.723 | 29.826 | 34.760 | 1.00 | 42.59 |
| ATOM | 579 | N | VAL | 88 | 3.479 | 29.876 | 36.868 | 1.00 | 42.19 |
| ATOM | 580 | CA | VAL | 88 | 3.631 | 28.431 | 36.961 | 1.00 | 42.00 |
| ATOM | 581 | CB | VAL | 88 | 4.668 | 28.057 | 38.043 | 1.00 | 41.45 |
| ATOM | 582 | CG1 | VAL | 88 | 4.908 | 26.555 | 38.039 | 1.00 | 41.94 |
| ATOM | 583 | CG2 | VAL | 88 | 5.952 | 28.804 | 37.802 | 1.00 | 40.44 |
| ATOM | 584 | C | VAL | 88 | 2.346 | 27.693 | 37.271 | 1.00 | 41.90 |
| ATOM | 585 | O | VAL | 88 | 1.694 | 27.967 | 38.265 | 1.00 | 41.91 |
| ATOM | 586 | N | THR | 89 | 2.001 | 26.737 | 36.419 | 1.00 | 42.31 |
| ATOM | 587 | CA | THR | 89 | 0.799 | 25.929 | 36.602 | 1.00 | 43.07 |
| ATOM | 588 | CB | THR | 89 | −0.196 | 26.129 | 35.470 | 1.00 | 42.64 |
| ATOM | 589 | OG1 | THR | 89 | 0.337 | 25.540 | 34.279 | 1.00 | 40.99 |
| ATOM | 590 | CG2 | THR | 89 | −0.460 | 27.613 | 35.247 | 1.00 | 42.22 |
| ATOM | 591 | C | THR | 89 | 1.218 | 24.470 | 36.551 | 1.00 | 43.77 |
| ATOM | 592 | O | THR | 89 | 2.358 | 24.165 | 36.214 | 1.00 | 44.96 |
| ATOM | 593 | N | ASP | 90 | 0.297 | 23.564 | 36.856 | 1.00 | 43.62 |
| ATOM | 594 | CA | ASP | 90 | 0.612 | 22.142 | 36.836 | 1.00 | 43.36 |
| ATOM | 595 | CB | ASP | 90 | −0.571 | 21.337 | 37.382 | 1.00 | 44.19 |
| ATOM | 596 | CG | ASP | 90 | −1.848 | 21.546 | 36.571 | 1.00 | 46.79 |
| ATOM | 597 | OD1 | ASP | 90 | −1.899 | 22.500 | 35.756 | 1.00 | 47.51 |
| ATOM | 598 | OD2 | ASP | 90 | −2.809 | 20.760 | 36.758 | 1.00 | 48.03 |
| ATOM | 599 | C | ASP | 90 | 0.975 | 21.649 | 35.437 | 1.00 | 42.81 |
| ATOM | 600 | O | ASP | 90 | 1.440 | 20.521 | 35.273 | 1.00 | 43.66 |
| ATOM | 601 | N | GLU | 91 | 0.766 | 22.483 | 34.424 | 1.00 | 41.50 |
| ATOM | 602 | CA | GLU | 91 | 1.091 | 22.086 | 33.054 | 1.00 | 40.04 |
| ATOM | 603 | CB | GLU | 91 | 0.076 | 22.672 | 32.069 | 1.00 | 39.51 |
| ATOM | 604 | CG | GLU | 91 | −1.329 | 22.109 | 32.215 | 1.00 | 39.37 |
| ATOM | 605 | CD | GLU | 91 | −2.313 | 22.698 | 31.208 | 1.00 | 39.86 |
| ATOM | 606 | OE1 | GLU | 91 | −2.338 | 23.935 | 31.041 | 1.00 | 40.41 |
| ATOM | 607 | OE2 | GLU | 91 | −3.072 | 21.929 | 30.590 | 1.00 | 39.43 |
| ATOM | 608 | C | GLU | 91 | 2.496 | 22.527 | 32.659 | 1.00 | 38.71 |
| ATOM | 609 | O | GLU | 91 | 2.880 | 22.438 | 31.495 | 1.00 | 38.40 |
| ATOM | 610 | N | CYS | 92 | 3.261 | 22.995 | 33.638 | 1.00 | 36.91 |
| ATOM | 611 | CA | CYS | 92 | 4.614 | 23.469 | 33.384 | 1.00 | 35.73 |
| ATOM | 612 | CB | CYS | 92 | 4.811 | 24.838 | 34.036 | 1.00 | 35.48 |
| ATOM | 613 | SG | CYS | 92 | 3.619 | 26.089 | 33.511 | 1.00 | 33.42 |
| ATOM | 614 | C | CYS | 92 | 5.693 | 22.519 | 33.886 | 1.00 | 34.96 |
| ATOM | 615 | O | CYS | 92 | 6.876 | 22.863 | 33.873 | 1.00 | 34.46 |
| ATOM | 616 | N | PHE | 93 | 5.288 | 21.328 | 34.323 | 1.00 | 34.01 |
| ATOM | 617 | CA | PHE | 93 | 6.241 | 20.357 | 34.847 | 1.00 | 33.41 |
| ATOM | 618 | CB | PHE | 93 | 5.841 | 19.973 | 36.274 | 1.00 | 33.36 |
| ATOM | 619 | CG | PHE | 93 | 5.797 | 21.147 | 37.217 | 1.00 | 33.71 |
| ATOM | 620 | CD1 | PHE | 93 | 6.973 | 21.800 | 37.593 | 1.00 | 34.28 |
| ATOM | 621 | CD2 | PHE | 93 | 4.582 | 21.634 | 37.694 | 1.00 | 33.99 |
| ATOM | 622 | CE1 | PHE | 93 | 6.941 | 22.928 | 38.431 | 1.00 | 33.93 |
| ATOM | 623 | CE2 | PHE | 93 | 4.537 | 22.757 | 38.529 | 1.00 | 33.44 |
| ATOM | 624 | CZ | PHE | 93 | 5.720 | 23.404 | 38.896 | 1.00 | 33.83 |
| ATOM | 625 | C | PHE | 93 | 6.394 | 19.122 | 33.968 | 1.00 | 32.60 |
| ATOM | 626 | O | PHE | 93 | 5.410 | 18.565 | 33.470 | 1.00 | 32.56 |
| ATOM | 627 | N | PHE | 94 | 7.644 | 18.710 | 33.779 | 1.00 | 31.35 |
| ATOM | 628 | CA | PHE | 94 | 7.956 | 17.570 | 32.933 | 1.00 | 30.20 |
| ATOM | 629 | CB | PHE | 94 | 8.574 | 18.053 | 31.630 | 1.00 | 29.25 |
| ATOM | 630 | CG | PHE | 94 | 7.761 | 19.088 | 30.936 | 1.00 | 28.30 |
| ATOM | 631 | CD1 | PHE | 94 | 6.778 | 18.717 | 30.020 | 1.00 | 27.94 |
| ATOM | 632 | CD2 | PHE | 94 | 7.917 | 20.432 | 31.254 | 1.00 | 26.64 |
| ATOM | 633 | CE1 | PHE | 94 | 5.961 | 19.674 | 29.440 | 1.00 | 28.07 |
| ATOM | 634 | CE2 | PHE | 94 | 7.107 | 21.388 | 30.683 | 1.00 | 26.92 |
| ATOM | 635 | CZ | PHE | 94 | 6.125 | 21.014 | 29.775 | 1.00 | 26.80 |
| ATOM | 636 | C | PHE | 94 | 8.930 | 16.624 | 33.585 | 1.00 | 30.36 |
| ATOM | 637 | O | PHE | 94 | 9.768 | 17.041 | 34.387 | 1.00 | 30.51 |
| ATOM | 638 | N | PHE | 95 | 8.815 | 15.344 | 33.242 | 1.00 | 30.11 |
| ATOM | 639 | CA | PHE | 95 | 9.736 | 14.345 | 33.757 | 1.00 | 29.83 |
| ATOM | 640 | CB | PHE | 95 | 9.161 | 12.933 | 33.663 | 1.00 | 30.35 |
| ATOM | 641 | CG | PHE | 95 | 7.882 | 12.735 | 34.417 | 1.00 | 31.10 |
| ATOM | 642 | CD1 | PHE | 95 | 6.679 | 12.586 | 33.733 | 1.00 | 31.54 |
| ATOM | 643 | CD2 | PHE | 95 | 7.876 | 12.690 | 35.807 | 1.00 | 30.85 |
| ATOM | 644 | CE1 | PHE | 95 | 5.488 | 12.394 | 34.422 | 1.00 | 32.11 |
| ATOM | 645 | CE2 | PHE | 95 | 6.692 | 12.499 | 36.508 | 1.00 | 31.21 |
| ATOM | 646 | CZ | PHE | 95 | 5.493 | 12.351 | 35.815 | 1.00 | 31.97 |
| ATOM | 647 | C | PHE | 95 | 10.933 | 14.432 | 32.826 | 1.00 | 29.77 |
| ATOM | 648 | O | PHE | 95 | 10.807 | 14.231 | 31.616 | 1.00 | 30.05 |
| ATOM | 649 | N | GLU | 96 | 12.087 | 14.763 | 33.384 | 1.00 | 29.61 |
| ATOM | 650 | CA | GLU | 96 | 13.301 | 14.856 | 32.599 | 1.00 | 29.17 |
| ATOM | 651 | CB | GLU | 96 | 14.217 | 15.960 | 33.131 | 1.00 | 28.66 |
| ATOM | 652 | CG | GLU | 96 | 15.555 | 16.033 | 32.401 | 1.00 | 28.32 |
| ATOM | 653 | CD | GLU | 96 | 16.507 | 17.072 | 32.972 | 1.00 | 28.28 |
| ATOM | 654 | OE1 | GLU | 96 | 16.830 | 17.003 | 34.176 | 1.00 | 28.47 |
| ATOM | 655 | OE2 | GLU | 96 | 16.949 | 17.957 | 32.213 | 1.00 | 29.85 |
| ATOM | 656 | C | GLU | 96 | 14.019 | 13.524 | 32.701 | 1.00 | 29.36 |
| ATOM | 657 | O | GLU | 96 | 14.392 | 13.097 | 33.791 | 1.00 | 29.94 |
| ATOM | 658 | N | ARG | 97 | 14.211 | 12.865 | 31.568 | 1.00 | 29.12 |
| ATOM | 659 | CA | ARG | 97 | 14.899 | 11.589 | 31.569 | 1.00 | 28.94 |
| ATOM | 660 | CB | ARG | 97 | 13.942 | 10.464 | 31.176 | 1.00 | 30.10 |
| ATOM | 661 | CG | ARG | 97 | 14.557 | 9.084 | 31.295 | 1.00 | 32.11 |
| ATOM | 662 | CD | ARG | 97 | 13.709 | 8.004 | 30.615 | 1.00 | 34.80 |
| ATOM | 663 | NE | ARG | 97 | 14.268 | 6.657 | 30.783 | 1.00 | 36.88 |
| ATOM | 664 | CZ | ARG | 97 | 14.296 | 5.988 | 31.939 | 1.00 | 38.16 |
| ATOM | 665 | NH1 | ARG | 97 | 13.795 | 6.528 | 33.046 | 1.00 | 38.69 |
| ATOM | 666 | NH2 | ARG | 97 | 14.829 | 4.774 | 31.992 | 1.00 | 39.07 |
| ATOM | 667 | C | ARG | 97 | 16.100 | 11.551 | 30.636 | 1.00 | 27.85 |
| ATOM | 668 | O | ARG | 97 | 16.029 | 11.979 | 29.489 | 1.00 | 27.26 |
| ATOM | 669 | N | LEU | 98 | 17.209 | 11.052 | 31.162 | 1.00 | 26.65 |
| ATOM | 670 | CA | LEU | 98 | 18.417 | 10.890 | 30.388 | 1.00 | 26.16 |
| ATOM | 671 | CB | LEU | 98 | 19.662 | 10.983 | 31.283 | 1.00 | 25.10 |
| ATOM | 672 | CG | LEU | 98 | 20.922 | 10.397 | 30.639 | 1.00 | 23.67 |
| ATOM | 673 | CD1 | LEU | 98 | 21.103 | 10.977 | 29.239 | 1.00 | 22.19 |
| ATOM | 674 | CD2 | LEU | 98 | 22.124 | 10.663 | 31.520 | 1.00 | 23.41 |
| ATOM | 675 | C | LEU | 98 | 18.256 | 9.478 | 29.837 | 1.00 | 26.51 |
| ATOM | 676 | O | LEU | 98 | 18.473 | 8.488 | 30.537 | 1.00 | 26.14 |
| ATOM | 677 | N | GLU | 99 | 17.848 | 9.393 | 28.581 | 1.00 | 26.80 |
| ATOM | 678 | CA | GLU | 99 | 17.622 | 8.115 | 27.936 | 1.00 | 26.82 |
| ATOM | 679 | CB | GLU | 99 | 16.927 | 8.348 | 26.603 | 1.00 | 26.24 |
| ATOM | 680 | CG | GLU | 99 | 15.639 | 9.147 | 26.718 | 1.00 | 28.42 |
| ATOM | 681 | CD | GLU | 99 | 14.450 | 8.315 | 27.191 | 1.00 | 28.95 |
| ATOM | 682 | OE1 | GLU | 99 | 13.337 | 8.879 | 27.350 | 1.00 | 28.29 |
| ATOM | 683 | OE2 | GLU | 99 | 14.627 | 7.096 | 27.399 | 1.00 | 30.66 |
| ATOM | 684 | C | GLU | 99 | 18.915 | 7.341 | 27.719 | 1.00 | 27.08 |
| ATOM | 685 | O | GLU | 99 | 20.008 | 7.902 | 27.759 | 1.00 | 27.12 |
| ATOM | 686 | N | SER | 100 | 18.775 | 6.048 | 27.469 | 1.00 | 27.05 |
| ATOM | 687 | CA | SER | 100 | 19.921 | 5.192 | 27.247 | 1.00 | 27.22 |
| ATOM | 688 | CB | SER | 100 | 19.476 | 3.744 | 27.150 | 1.00 | 28.27 |
| ATOM | 689 | OG | SER | 100 | 18.748 | 3.559 | 25.957 | 1.00 | 31.47 |
| ATOM | 690 | C | SER | 100 | 20.697 | 5.565 | 25.993 | 1.00 | 26.70 |
| ATOM | 691 | O | SER | 100 | 21.835 | 5.147 | 25.830 | 1.00 | 26.34 |
| ATOM | 692 | N | ASN | 101 | 20.093 | 6.337 | 25.096 | 1.00 | 26.70 |
| ATOM | 693 | CA | ASN | 101 | 20.813 | 6.746 | 23.886 | 1.00 | 25.33 |
| ATOM | 694 | CB | ASN | 101 | 19.866 | 6.891 | 22.709 | 1.00 | 25.22 |
| ATOM | 695 | CG | ASN | 101 | 19.005 | 8.107 | 22.826 | 1.00 | 26.50 |
| ATOM | 696 | OD1 | ASN | 101 | 18.848 | 8.668 | 23.916 | 1.00 | 26.62 |
| ATOM | 697 | ND2 | ASN | 101 | 18.426 | 8.529 | 21.709 | 1.00 | 27.53 |
| ATOM | 698 | C | ASN | 101 | 21.540 | 8.071 | 24.108 | 1.00 | 24.54 |
| ATOM | 699 | O | ASN | 101 | 22.061 | 8.662 | 23.175 | 1.00 | 24.37 |
| ATOM | 700 | N | ASN | 102 | 21.566 | 8.514 | 25.361 | 1.00 | 24.45 |
| ATOM | 701 | CA | ASN | 102 | 22.213 | 9.755 | 25.808 | 1.00 | 24.46 |
| ATOM | 702 | CB | ASN | 102 | 23.698 | 9.785 | 25.450 | 1.00 | 23.96 |
| ATOM | 703 | CG | ASN | 102 | 24.512 | 8.820 | 26.292 | 1.00 | 25.70 |
| ATOM | 704 | OD1 | ASN | 102 | 24.287 | 8.676 | 27.493 | 1.00 | 26.34 |
| ATOM | 705 | ND2 | ASN | 102 | 25.467 | 8.151 | 25.663 | 1.00 | 27.30 |
| ATOM | 706 | C | ASN | 102 | 21.566 | 11.073 | 25.432 | 1.00 | 24.22 |
| ATOM | 707 | O | ASN | 102 | 22.197 | 12.122 | 25.470 | 1.00 | 24.68 |
| ATOM | 708 | N | TYR | 103 | 20.297 | 11.018 | 25.077 | 1.00 | 24.01 |
| ATOM | 709 | CA | TYR | 103 | 19.561 | 12.229 | 24.788 | 1.00 | 24.03 |
| ATOM | 710 | CB | TYR | 103 | 18.867 | 12.112 | 23.443 | 1.00 | 23.95 |
| ATOM | 711 | CG | TYR | 103 | 19.776 | 12.339 | 22.254 | 1.00 | 24.32 |
| ATOM | 712 | CD1 | TYR | 103 | 19.956 | 13.621 | 21.722 | 1.00 | 22.76 |
| ATOM | 713 | CE1 | TYR | 103 | 20.710 | 13.822 | 20.584 | 1.00 | 23.37 |
| ATOM | 714 | CD2 | TYR | 103 | 20.395 | 11.262 | 21.615 | 1.00 | 23.85 |
| ATOM | 715 | CE2 | TYR | 103 | 21.158 | 11.454 | 20.465 | 1.00 | 24.34 |
| ATOM | 716 | CZ | TYR | 103 | 21.304 | 12.734 | 19.956 | 1.00 | 24.60 |
| ATOM | 717 | OH | TYR | 103 | 22.012 | 12.908 | 18.794 | 1.00 | 27.78 |
| ATOM | 718 | C | TYR | 103 | 18.539 | 12.346 | 25.924 | 1.00 | 24.04 |
| ATOM | 719 | O | TYR | 103 | 18.246 | 11.367 | 26.612 | 1.00 | 23.70 |
| ATOM | 720 | N | ASN | 104 | 18.026 | 13.545 | 26.149 | 1.00 | 24.29 |
| ATOM | 721 | CA | ASN | 104 | 17.036 | 13.752 | 27.192 | 1.00 | 24.08 |
| ATOM | 722 | CB | ASN | 104 | 17.300 | 15.056 | 27.923 | 1.00 | 24.06 |
| ATOM | 723 | CG | ASN | 104 | 18.481 | 14.977 | 28.858 | 1.00 | 24.66 |
| ATOM | 724 | OD1 | ASN | 104 | 19.305 | 14.056 | 28.785 | 1.00 | 23.53 |
| ATOM | 725 | ND2 | ASN | 104 | 18.580 | 15.961 | 29.745 | 1.00 | 24.15 |
| ATOM | 726 | C | ASN | 104 | 15.662 | 13.828 | 26.570 | 1.00 | 24.42 |
| ATOM | 727 | O | ASN | 104 | 15.516 | 14.204 | 25.410 | 1.00 | 24.35 |
| ATOM | 728 | N | THR | 105 | 14.653 | 13.438 | 27.334 | 1.00 | 25.04 |
| ATOM | 729 | CA | THR | 105 | 13.268 | 13.530 | 26.887 | 1.00 | 25.02 |
| ATOM | 730 | CB | THR | 105 | 12.552 | 12.147 | 26.727 | 1.00 | 24.65 |
| ATOM | 731 | OG1 | THR | 105 | 12.721 | 11.354 | 27.909 | 1.00 | 24.73 |
| ATOM | 732 | CG2 | THR | 105 | 13.069 | 11.406 | 25.510 | 1.00 | 23.90 |
| ATOM | 733 | C | THR | 105 | 12.557 | 14.313 | 27.973 | 1.00 | 25.71 |
| ATOM | 734 | O | THR | 105 | 13.003 | 14.350 | 29.113 | 1.00 | 25.75 |
| ATOM | 735 | N | TYR | 106 | 11.462 | 14.955 | 27.613 | 1.00 | 26.81 |
| ATOM | 736 | CA | TYR | 106 | 10.694 | 15.730 | 28.570 | 1.00 | 27.94 |
| ATOM | 737 | CB | TYR | 106 | 10.933 | 17.211 | 28.330 | 1.00 | 27.66 |
| ATOM | 738 | CG | TYR | 106 | 12.350 | 17.580 | 28.653 | 1.00 | 27.77 |
| ATOM | 739 | CD1 | TYR | 106 | 12.738 | 17.805 | 29.964 | 1.00 | 28.19 |
| ATOM | 740 | CE1 | TYR | 106 | 14.058 | 18.086 | 30.287 | 1.00 | 28.26 |
| ATOM | 741 | CD2 | TYR | 106 | 13.321 | 17.646 | 27.656 | 1.00 | 28.40 |
| ATOM | 742 | CE2 | TYR | 106 | 14.656 | 17.927 | 27.966 | 1.00 | 28.15 |
| ATOM | 743 | CZ | TYR | 106 | 15.015 | 18.145 | 29.289 | 1.00 | 28.55 |
| ATOM | 744 | OH | TYR | 106 | 16.330 | 18.405 | 29.630 | 1.00 | 28.73 |
| ATOM | 745 | C | TYR | 106 | 9.238 | 15.365 | 28.410 | 1.00 | 28.19 |
| ATOM | 746 | O | TYR | 106 | 8.589 | 15.741 | 27.442 | 1.00 | 27.73 |
| ATOM | 747 | N | ARG | 107 | 8.741 | 14.600 | 29.372 | 1.00 | 29.47 |
| ATOM | 748 | CA | ARG | 107 | 7.372 | 14.124 | 29.344 | 1.00 | 30.69 |
| ATOM | 749 | CB | ARG | 107 | 7.379 | 12.646 | 29.717 | 1.00 | 30.07 |
| ATOM | 750 | CG | ARG | 107 | 6.085 | 11.910 | 29.493 | 1.00 | 31.65 |
| ATOM | 751 | CD | ARG | 107 | 6.338 | 10.415 | 29.435 | 1.00 | 31.97 |
| ATOM | 752 | NE | ARG | 107 | 6.993 | 9.897 | 30.633 | 1.00 | 33.36 |
| ATOM | 753 | CZ | ARG | 107 | 6.377 | 9.684 | 31.794 | 1.00 | 34.07 |
| ATOM | 754 | NH1 | ARG | 107 | 5.084 | 9.946 | 31.914 | 1.00 | 36.13 |
| ATOM | 755 | NH2 | ARG | 107 | 7.048 | 9.208 | 32.833 | 1.00 | 33.89 |
| ATOM | 756 | C | ARG | 107 | 6.470 | 14.932 | 30.276 | 1.00 | 32.10 |
| ATOM | 757 | O | ARG | 107 | 6.820 | 15.195 | 31.425 | 1.00 | 32.07 |
| ATOM | 758 | N | SER | 108 | 5.313 | 15.340 | 29.766 | 1.00 | 33.50 |
| ATOM | 759 | CA | SER | 108 | 4.364 | 16.119 | 30.553 | 1.00 | 34.86 |
| ATOM | 760 | CB | SER | 108 | 3.127 | 16.439 | 29.718 | 1.00 | 34.70 |
| ATOM | 761 | OG | SER | 108 | 2.098 | 16.990 | 30.521 | 1.00 | 34.38 |
| ATOM | 762 | C | SER | 108 | 3.933 | 15.356 | 31.793 | 1.00 | 36.33 |
| ATOM | 763 | O | SER | 108 | 3.509 | 14.205 | 31.698 | 1.00 | 36.69 |
| ATOM | 764 | N | ARG | 109 | 4.039 | 15.988 | 32.959 | 1.00 | 37.92 |
| ATOM | 765 | CA | ARG | 109 | 3.623 | 15.326 | 34.187 | 1.00 | 39.14 |
| ATOM | 766 | CB | ARG | 109 | 4.149 | 16.060 | 35.417 | 1.00 | 40.63 |
| ATOM | 767 | CG | ARG | 109 | 3.465 | 15.578 | 36.683 | 1.00 | 42.04 |
| ATOM | 768 | CD | ARG | 109 | 4.342 | 15.650 | 37.889 | 1.00 | 42.71 |
| ATOM | 769 | NE | ARG | 109 | 4.553 | 17.016 | 38.329 | 1.00 | 44.73 |
| ATOM | 770 | CZ | ARG | 109 | 4.542 | 17.384 | 39.606 | 1.00 | 46.16 |
| ATOM | 771 | NH1 | ARG | 109 | 4.324 | 16.466 | 40.549 | 1.00 | 46.18 |
| ATOM | 772 | NH2 | ARG | 109 | 4.763 | 18.658 | 39.938 | 1.00 | 45.68 |
| ATOM | 773 | C | ARG | 109 | 2.099 | 15.275 | 34.259 | 1.00 | 39.59 |
| ATOM | 774 | O | ARG | 109 | 1.528 | 14.424 | 34.944 | 1.00 | 39.76 |
| ATOM | 775 | N | LYS | 110 | 1.449 | 16.196 | 33.556 | 1.00 | 39.43 |
| ATOM | 776 | CA | LYS | 110 | 0.003 | 16.242 | 33.538 | 1.00 | 39.87 |
| ATOM | 777 | CB | LYS | 110 | −0.482 | 17.663 | 33.272 | 1.00 | 41.21 |
| ATOM | 778 | CG | LYS | 110 | −1.903 | 17.902 | 33.756 | 1.00 | 43.57 |
| ATOM | 779 | CD | LYS | 110 | −2.204 | 19.393 | 33.883 | 1.00 | 45.66 |
| ATOM | 780 | CE | LYS | 110 | −3.689 | 19.667 | 34.144 | 1.00 | 46.43 |
| ATOM | 781 | NZ | LYS | 110 | −4.196 | 18.973 | 35.367 | 1.00 | 46.96 |
| ATOM | 782 | C | LYS | 110 | −0.530 | 15.282 | 32.477 | 1.00 | 39.50 |
| ATOM | 783 | O | LYS | 110 | −1.397 | 14.460 | 32.763 | 1.00 | 39.31 |
| ATOM | 784 | N | TYR | 111 | 0.002 | 15.381 | 31.258 | 1.00 | 38.61 |
| ATOM | 785 | CA | TYR | 111 | −0.391 | 14.509 | 30.149 | 1.00 | 37.47 |
| ATOM | 786 | CB | TYR | 111 | −0.594 | 15.348 | 28.903 | 1.00 | 36.55 |
| ATOM | 787 | CG | TYR | 111 | −1.489 | 16.520 | 29.199 | 1.00 | 37.11 |
| ATOM | 788 | CD1 | TYR | 111 | −2.792 | 16.313 | 29.657 | 1.00 | 36.98 |
| ATOM | 789 | CE1 | TYR | 111 | −3.602 | 17.368 | 30.027 | 1.00 | 36.28 |
| ATOM | 790 | CD2 | TYR | 111 | −1.022 | 17.832 | 29.109 | 1.00 | 36.58 |
| ATOM | 791 | CE2 | TYR | 111 | −1.835 | 18.907 | 29.479 | 1.00 | 36.57 |
| ATOM | 792 | CZ | TYR | 111 | −3.127 | 18.658 | 29.944 | 1.00 | 36.66 |
| ATOM | 793 | OH | TYR | 111 | −3.935 | 19.684 | 30.380 | 1.00 | 36.97 |
| ATOM | 794 | C | TYR | 111 | 0.736 | 13.501 | 29.957 | 1.00 | 37.25 |
| ATOM | 795 | O | TYR | 111 | 1.481 | 13.542 | 28.983 | 1.00 | 36.98 |
| ATOM | 796 | N | THR | 112 | 0.822 | 12.589 | 30.918 | 1.00 | 37.10 |
| ATOM | 797 | CA | THR | 112 | 1.858 | 11.570 | 31.009 | 1.00 | 37.05 |
| ATOM | 798 | CB | THR | 112 | 1.515 | 10.526 | 32.099 | 1.00 | 36.65 |
| ATOM | 799 | OG1 | THR | 112 | 0.503 | 9.639 | 31.618 | 1.00 | 35.35 |
| ATOM | 800 | CG2 | THR | 112 | 1.016 | 11.219 | 33.361 | 1.00 | 36.32 |
| ATOM | 801 | C | THR | 112 | 2.329 | 10.806 | 29.789 | 1.00 | 37.20 |
| ATOM | 802 | O | THR | 112 | 3.344 | 10.128 | 29.867 | 1.00 | 37.95 |
| ATOM | 803 | N | SER | 113 | 1.637 | 10.879 | 28.664 | 1.00 | 37.09 |
| ATOM | 804 | CA | SER | 113 | 2.149 | 10.140 | 27.520 | 1.00 | 36.38 |
| ATOM | 805 | CB | SER | 113 | 1.149 | 9.081 | 27.041 | 1.00 | 36.67 |
| ATOM | 806 | OG | SER | 113 | 0.040 | 9.665 | 26.400 | 1.00 | 38.16 |
| ATOM | 807 | C | SER | 113 | 2.557 | 11.049 | 26.374 | 1.00 | 35.98 |
| ATOM | 808 | O | SER | 113 | 2.828 | 10.584 | 25.270 | 1.00 | 36.20 |
| ATOM | 809 | N | TRP | 114 | 2.619 | 12.347 | 26.637 | 1.00 | 35.33 |
| ATOM | 810 | CA | TRP | 114 | 3.020 | 13.283 | 25.601 | 1.00 | 35.24 |
| ATOM | 811 | CB | TRP | 114 | 1.953 | 14.364 | 25.422 | 1.00 | 36.67 |
| ATOM | 812 | CG | TRP | 114 | 0.598 | 13.828 | 25.070 | 1.00 | 38.36 |
| ATOM | 813 | CD2 | TRP | 114 | −0.646 | 14.534 | 25.142 | 1.00 | 39.09 |
| ATOM | 814 | CE2 | TRP | 114 | −1.650 | 13.665 | 24.657 | 1.00 | 40.04 |
| ATOM | 815 | CE3 | TRP | 114 | −1.010 | 15.817 | 25.567 | 1.00 | 39.40 |
| ATOM | 816 | CD1 | TRP | 114 | 0.306 | 12.592 | 24.564 | 1.00 | 38.75 |
| ATOM | 817 | NE1 | TRP | 114 | −1.043 | 12.486 | 24.313 | 1.00 | 39.11 |
| ATOM | 818 | CZ2 | TRP | 114 | −2.997 | 14.043 | 24.584 | 1.00 | 40.56 |
| ATOM | 819 | CZ3 | TRP | 114 | −2.339 | 16.191 | 25.496 | 1.00 | 40.33 |
| ATOM | 820 | CH2 | TRP | 114 | −3.320 | 15.306 | 25.007 | 1.00 | 40.55 |
| ATOM | 821 | C | TRP | 114 | 4.377 | 13.916 | 25.917 | 1.00 | 34.10 |
| ATOM | 822 | O | TRP | 114 | 4.669 | 14.245 | 27.071 | 1.00 | 33.96 |
| ATOM | 823 | N | TYR | 115 | 5.199 | 14.076 | 24.883 | 1.00 | 32.52 |
| ATOM | 824 | CA | TYR | 115 | 6.529 | 14.645 | 25.032 | 1.00 | 30.94 |
| ATOM | 825 | CB | TYR | 115 | 7.580 | 13.750 | 24.385 | 1.00 | 30.98 |
| ATOM | 826 | CG | TYR | 115 | 7.739 | 12.383 | 24.977 | 1.00 | 30.54 |
| ATOM | 827 | CD1 | TYR | 115 | 6.887 | 11.347 | 24.616 | 1.00 | 29.82 |
| ATOM | 828 | CE1 | TYR | 115 | 7.071 | 10.075 | 25.113 | 1.00 | 31.32 |
| ATOM | 829 | CD2 | TYR | 115 | 8.784 | 12.111 | 25.862 | 1.00 | 30.94 |
| ATOM | 830 | CE2 | TYR | 115 | 8.981 | 10.838 | 26.373 | 1.00 | 30.96 |
| ATOM | 831 | CZ | TYR | 115 | 8.123 | 9.824 | 25.994 | 1.00 | 31.82 |
| ATOM | 832 | OH | TYR | 115 | 8.313 | 8.559 | 26.494 | 1.00 | 32.48 |
| ATOM | 833 | C | TYR | 115 | 6.671 | 16.000 | 24.379 | 1.00 | 30.22 |
| ATOM | 834 | O | TYR | 115 | 5.950 | 16.328 | 23.433 | 1.00 | 30.13 |
| ATOM | 835 | N | VAL | 116 | 7.623 | 16.775 | 24.886 | 1.00 | 29.29 |
| ATOM | 836 | CA | VAL | 116 | 7.936 | 18.072 | 24.315 | 1.00 | 29.10 |
| ATOM | 837 | CB | VAL | 116 | 8.899 | 18.852 | 25.229 | 1.00 | 28.31 |
| ATOM | 838 | CG1 | VAL | 116 | 9.291 | 20.164 | 24.578 | 1.00 | 26.24 |
| ATOM | 839 | CG2 | VAL | 116 | 8.248 | 19.066 | 26.585 | 1.00 | 26.68 |
| ATOM | 840 | C | VAL | 116 | 8.654 | 17.670 | 23.030 | 1.00 | 29.31 |
| ATOM | 841 | O | VAL | 116 | 9.476 | 16.754 | 23.044 | 1.00 | 28.94 |
| ATOM | 842 | N | ALA | 117 | 8.352 | 18.333 | 21.924 | 1.00 | 29.95 |
| ATOM | 843 | CA | ALA | 117 | 8.973 | 17.948 | 20.671 | 1.00 | 30.87 |
| ATOM | 844 | CB | ALA | 117 | 8.309 | 16.673 | 20.157 | 1.00 | 29.42 |
| ATOM | 845 | C | ALA | 117 | 8.935 | 19.024 | 19.598 | 1.00 | 32.06 |
| ATOM | 846 | O | ALA | 117 | 8.065 | 19.890 | 19.599 | 1.00 | 32.32 |
| ATOM | 847 | N | LEU | 118 | 9.896 | 18.956 | 18.680 | 1.00 | 33.64 |
| ATOM | 848 | CA | LEU | 118 | 9.991 | 19.904 | 17.575 | 1.00 | 34.88 |
| ATOM | 849 | CB | LEU | 118 | 11.274 | 20.720 | 17.681 | 1.00 | 33.49 |
| ATOM | 850 | CG | LEU | 118 | 11.348 | 21.637 | 18.897 | 1.00 | 33.25 |
| ATOM | 851 | CD1 | LEU | 118 | 12.694 | 22.367 | 18.912 | 1.00 | 32.56 |
| ATOM | 852 | CD2 | LEU | 118 | 10.192 | 22.616 | 18.852 | 1.00 | 32.02 |
| ATOM | 853 | C | LEU | 118 | 10.000 | 19.149 | 16.262 | 1.00 | 36.58 |
| ATOM | 854 | O | LEU | 118 | 10.614 | 18.091 | 16.162 | 1.00 | 37.96 |
| ATOM | 855 | N | LYS | 119 | 9.312 | 19.680 | 15.258 | 1.00 | 38.16 |
| ATOM | 856 | CA | LYS | 119 | 9.292 | 19.037 | 13.954 | 1.00 | 39.70 |
| ATOM | 857 | CB | LYS | 119 | 8.028 | 19.413 | 13.184 | 1.00 | 40.59 |
| ATOM | 858 | CG | LYS | 119 | 6.775 | 18.704 | 13.671 | 1.00 | 41.77 |
| ATOM | 859 | CD | LYS | 119 | 5.597 | 18.941 | 12.729 | 1.00 | 42.98 |
| ATOM | 860 | CE | LYS | 119 | 5.317 | 20.419 | 12.596 | 1.00 | 44.14 |
| ATOM | 861 | NZ | LYS | 119 | 5.330 | 21.051 | 13.947 | 1.00 | 45.69 |
| ATOM | 862 | C | LYS | 119 | 10.531 | 19.456 | 13.168 | 1.00 | 40.77 |
| ATOM | 863 | O | LYS | 119 | 11.270 | 20.345 | 13.588 | 1.00 | 40.35 |
| ATOM | 864 | N | ARG | 120 | 10.761 | 18.810 | 12.031 | 1.00 | 42.75 |
| ATOM | 865 | CA | ARG | 120 | 11.912 | 19.136 | 11.191 | 1.00 | 44.60 |
| ATOM | 866 | CB | ARG | 120 | 12.006 | 18.189 | 10.001 | 1.00 | 45.88 |
| ATOM | 867 | CG | ARG | 120 | 12.017 | 16.729 | 10.346 | 1.00 | 49.02 |
| ATOM | 868 | CD | ARG | 120 | 11.881 | 15.904 | 9.082 | 1.00 | 51.03 |
| ATOM | 869 | NE | ARG | 120 | 11.620 | 14.498 | 9.376 | 1.00 | 53.84 |
| ATOM | 870 | CZ | ARG | 120 | 11.224 | 13.609 | 8.467 | 1.00 | 55.46 |
| ATOM | 871 | NH1 | ARG | 120 | 11.044 | 13.991 | 7.205 | 1.00 | 56.37 |
| ATOM | 872 | NH2 | ARG | 120 | 10.999 | 12.343 | 8.817 | 1.00 | 55.68 |
| ATOM | 873 | C | ARG | 120 | 11.803 | 20.553 | 10.640 | 1.00 | 44.69 |
| ATOM | 874 | O | ARG | 120 | 12.772 | 21.087 | 10.110 | 1.00 | 45.06 |
| ATOM | 875 | N | THR | 121 | 10.622 | 21.156 | 10.746 | 1.00 | 44.32 |
| ATOM | 876 | CA | THR | 121 | 10.414 | 22.503 | 10.235 | 1.00 | 43.76 |
| ATOM | 877 | CB | THR | 121 | 8.949 | 22.754 | 9.866 | 1.00 | 43.55 |
| ATOM | 878 | OG1 | THR | 121 | 8.147 | 22.731 | 11.053 | 1.00 | 44.11 |
| ATOM | 879 | CG2 | THR | 121 | 8.455 | 21.697 | 8.905 | 1.00 | 42.91 |
| ATOM | 880 | C | THR | 121 | 10.803 | 23.562 | 11.242 | 1.00 | 44.11 |
| ATOM | 881 | O | THR | 121 | 10.855 | 24.744 | 10.915 | 1.00 | 44.87 |
| ATOM | 882 | N | GLY | 122 | 11.074 | 23.147 | 12.470 | 1.00 | 44.10 |
| ATOM | 883 | CA | GLY | 122 | 11.431 | 24.113 | 13.490 | 1.00 | 44.18 |
| ATOM | 884 | C | GLY | 122 | 10.212 | 24.511 | 14.301 | 1.00 | 43.87 |
| ATOM | 885 | O | GLY | 122 | 10.315 | 25.278 | 15.258 | 1.00 | 44.18 |
| ATOM | 886 | N | GLN | 123 | 9.050 | 24.000 | 13.907 | 1.00 | 43.28 |
| ATOM | 887 | CA | GLN | 123 | 7.805 | 24.273 | 14.615 | 1.00 | 43.15 |
| ATOM | 888 | CB | GLN | 123 | 6.612 | 24.204 | 13.668 | 1.00 | 43.26 |
| ATOM | 889 | CG | GLN | 123 | 6.719 | 25.115 | 12.486 | 1.00 | 44.87 |
| ATOM | 890 | CD | GLN | 123 | 6.914 | 26.550 | 12.903 | 1.00 | 45.57 |
| ATOM | 891 | OE1 | GLN | 123 | 6.051 | 27.138 | 13.553 | 1.00 | 44.96 |
| ATOM | 892 | NE2 | GLN | 123 | 8.062 | 27.124 | 12.538 | 1.00 | 46.77 |
| ATOM | 893 | C | GLN | 123 | 7.653 | 23.179 | 15.654 | 1.00 | 42.76 |
| ATOM | 894 | O | GLN | 123 | 8.057 | 22.044 | 15.417 | 1.00 | 42.89 |
| ATOM | 895 | N | TYR | 124 | 7.071 | 23.499 | 16.802 | 1.00 | 42.02 |
| ATOM | 896 | CA | TYR | 124 | 6.904 | 22.475 | 17.822 | 1.00 | 41.12 |
| ATOM | 897 | CB | TYR | 124 | 6.378 | 23.079 | 19.138 | 1.00 | 40.07 |
| ATOM | 898 | CG | TYR | 124 | 4.915 | 23.471 | 19.134 | 1.00 | 39.30 |
| ATOM | 899 | CD1 | TYR | 124 | 3.915 | 22.509 | 19.267 | 1.00 | 39.39 |
| ATOM | 900 | CE1 | TYR | 124 | 2.572 | 22.861 | 19.235 | 1.00 | 38.95 |
| ATOM | 901 | CD2 | TYR | 124 | 4.531 | 24.804 | 18.972 | 1.00 | 39.13 |
| ATOM | 902 | CE2 | TYR | 124 | 3.190 | 25.168 | 18.940 | 1.00 | 38.53 |
| ATOM | 903 | CZ | TYR | 124 | 2.215 | 24.192 | 19.068 | 1.00 | 38.81 |
| ATOM | 904 | OH | TYR | 124 | 0.881 | 24.537 | 19.008 | 1.00 | 38.77 |
| ATOM | 905 | C | TYR | 124 | 5.939 | 21.434 | 17.288 | 1.00 | 40.70 |
| ATOM | 906 | O | TYR | 124 | 5.169 | 21.703 | 16.379 | 1.00 | 40.39 |
| ATOM | 907 | N | LYS | 125 | 6.000 | 20.235 | 17.844 | 1.00 | 40.37 |
| ATOM | 908 | CA | LYS | 125 | 5.111 | 19.169 | 17.423 | 1.00 | 40.17 |
| ATOM | 909 | CB | LYS | 125 | 5.913 | 17.910 | 17.081 | 1.00 | 38.95 |
| ATOM | 910 | CG | LYS | 125 | 5.052 | 16.679 | 16.900 | 1.00 | 37.80 |
| ATOM | 911 | CD | LYS | 125 | 5.873 | 15.447 | 16.623 | 1.00 | 37.61 |
| ATOM | 912 | CE | LYS | 125 | 5.590 | 14.904 | 15.237 | 1.00 | 37.54 |
| ATOM | 913 | NZ | LYS | 125 | 6.280 | 13.601 | 15.011 | 1.00 | 37.60 |
| ATOM | 914 | C | LYS | 125 | 4.125 | 18.871 | 18.552 | 1.00 | 40.59 |
| ATOM | 915 | O | LYS | 125 | 4.519 | 18.743 | 19.715 | 1.00 | 41.45 |
| ATOM | 916 | N | LEU | 126 | 2.844 | 18.778 | 18.211 | 1.00 | 40.35 |
| ATOM | 917 | CA | LEU | 126 | 1.809 | 18.482 | 19.194 | 1.00 | 40.00 |
| ATOM | 918 | CB | LEU | 126 | 0.479 | 18.198 | 18.496 | 1.00 | 39.01 |
| ATOM | 919 | CG | LEU | 126 | −0.234 | 19.344 | 17.779 | 1.00 | 38.48 |
| ATOM | 920 | CD1 | LEU | 126 | −1.426 | 18.771 | 17.011 | 1.00 | 38.03 |
| ATOM | 921 | CD2 | LEU | 126 | −0.683 | 20.404 | 18.784 | 1.00 | 36.46 |
| ATOM | 922 | C | LEU | 126 | 2.172 | 17.278 | 20.053 | 1.00 | 40.33 |
| ATOM | 923 | O | LEU | 126 | 2.511 | 16.212 | 19.538 | 1.00 | 40.56 |
| ATOM | 924 | N | GLY | 127 | 2.093 | 17.449 | 21.366 | 1.00 | 40.46 |
| ATOM | 925 | CA | GLY | 127 | 2.404 | 16.353 | 22.264 | 1.00 | 40.66 |
| ATOM | 926 | C | GLY | 127 | 1.529 | 15.149 | 21.978 | 1.00 | 40.63 |
| ATOM | 927 | O | GLY | 127 | 1.954 | 14.009 | 22.125 | 1.00 | 39.82 |
| ATOM | 928 | N | SER | 128 | 0.298 | 15.413 | 21.555 | 1.00 | 40.87 |
| ATOM | 929 | CA | SER | 128 | −0.651 | 14.358 | 21.250 | 1.00 | 41.21 |
| ATOM | 930 | CB | SER | 128 | −1.991 | 14.975 | 20.900 | 1.00 | 40.69 |
| ATOM | 931 | OG | SER | 128 | −1.812 | 15.943 | 19.890 | 1.00 | 41.81 |
| ATOM | 932 | C | SER | 128 | −0.173 | 13.501 | 20.090 | 1.00 | 41.64 |
| ATOM | 933 | O | SER | 128 | −0.647 | 12.391 | 19.890 | 1.00 | 40.99 |
| ATOM | 934 | N | LYS | 129 | 0.772 | 14.024 | 19.321 | 1.00 | 42.40 |
| ATOM | 935 | CA | LYS | 129 | 1.295 | 13.288 | 18.186 | 1.00 | 42.92 |
| ATOM | 936 | CB | LYS | 129 | 1.267 | 14.180 | 16.942 | 1.00 | 43.84 |
| ATOM | 937 | CG | LYS | 129 | −0.141 | 14.329 | 16.387 | 1.00 | 45.32 |
| ATOM | 938 | CD | LYS | 129 | −0.260 | 15.387 | 15.307 | 1.00 | 47.16 |
| ATOM | 939 | CE | LYS | 129 | −1.710 | 15.461 | 14.812 | 1.00 | 48.88 |
| ATOM | 940 | NZ | LYS | 129 | −1.985 | 16.588 | 13.866 | 1.00 | 50.31 |
| ATOM | 941 | C | LYS | 129 | 2.690 | 12.719 | 18.426 | 1.00 | 42.38 |
| ATOM | 942 | O | LYS | 129 | 3.289 | 12.142 | 17.528 | 1.00 | 42.01 |
| ATOM | 943 | N | THR | 130 | 3.194 | 12.860 | 19.649 | 1.00 | 42.29 |
| ATOM | 944 | CA | THR | 130 | 4.523 | 12.354 | 19.983 | 1.00 | 41.66 |
| ATOM | 945 | CB | THR | 130 | 5.192 | 13.176 | 21.106 | 1.00 | 40.68 |
| ATOM | 946 | OG1 | THR | 130 | 4.489 | 12.962 | 22.334 | 1.00 | 39.45 |
| ATOM | 947 | CG2 | THR | 130 | 5.195 | 14.662 | 20.760 | 1.00 | 40.36 |
| ATOM | 948 | C | THR | 130 | 4.479 | 10.903 | 20.443 | 1.00 | 41.78 |
| ATOM | 949 | O | THR | 130 | 3.413 | 10.361 | 20.724 | 1.00 | 41.50 |
| ATOM | 950 | N | GLY | 131 | 5.655 | 10.286 | 20.518 | 1.00 | 42.00 |
| ATOM | 951 | CA | GLY | 131 | 5.758 | 8.904 | 20.946 | 1.00 | 41.91 |
| ATOM | 952 | C | GLY | 131 | 7.195 | 8.581 | 21.290 | 1.00 | 42.14 |
| ATOM | 953 | O | GLY | 131 | 8.095 | 9.317 | 20.895 | 1.00 | 42.22 |
| ATOM | 954 | N | PRO | 132 | 7.446 | 7.474 | 22.007 | 1.00 | 42.30 |
| ATOM | 955 | CD | PRO | 132 | 6.418 | 6.505 | 22.417 | 1.00 | 41.79 |
| ATOM | 956 | CA | PRO | 132 | 8.773 | 7.015 | 22.433 | 1.00 | 42.12 |
| ATOM | 957 | CB | PRO | 132 | 8.472 | 5.689 | 23.133 | 1.00 | 41.91 |
| ATOM | 958 | CG | PRO | 132 | 7.076 | 5.843 | 23.593 | 1.00 | 42.30 |
| ATOM | 959 | C | PRO | 132 | 9.775 | 6.813 | 21.300 | 1.00 | 42.01 |
| ATOM | 960 | O | PRO | 132 | 10.964 | 7.125 | 21.433 | 1.00 | 43.09 |
| ATOM | 961 | N | GLY | 133 | 9.296 | 6.273 | 20.188 | 1.00 | 41.52 |
| ATOM | 962 | CA | GLY | 133 | 10.188 | 6.016 | 19.074 | 1.00 | 41.10 |
| ATOM | 963 | C | GLY | 133 | 10.383 | 7.152 | 18.093 | 1.00 | 40.19 |
| ATOM | 964 | O | GLY | 133 | 10.687 | 6.910 | 16.931 | 1.00 | 40.19 |
| ATOM | 965 | N | GLN | 134 | 10.227 | 8.391 | 18.544 | 1.00 | 39.31 |
| ATOM | 966 | CA | GLN | 134 | 10.400 | 9.518 | 17.641 | 1.00 | 38.15 |
| ATOM | 967 | CB | GLN | 134 | 9.198 | 10.446 | 17.702 | 1.00 | 37.90 |
| ATOM | 968 | CG | GLN | 134 | 7.906 | 9.770 | 17.364 | 1.00 | 37.73 |
| ATOM | 969 | CD | GLN | 134 | 6.746 | 10.728 | 17.356 | 1.00 | 37.43 |
| ATOM | 970 | OE1 | GLN | 134 | 5.592 | 10.318 | 17.272 | 1.00 | 37.42 |
| ATOM | 971 | NE2 | GLN | 134 | 7.044 | 12.016 | 17.435 | 1.00 | 37.10 |
| ATOM | 972 | C | GLN | 134 | 11.654 | 10.323 | 17.910 | 1.00 | 37.85 |
| ATOM | 973 | O | GLN | 134 | 12.078 | 10.497 | 19.052 | 1.00 | 38.47 |
| ATOM | 974 | N | LYS | 135 | 12.236 | 10.822 | 16.833 | 1.00 | 36.65 |
| ATOM | 975 | CA | LYS | 135 | 13.443 | 11.623 | 16.883 | 1.00 | 35.45 |
| ATOM | 976 | CB | LYS | 135 | 14.025 | 11.660 | 15.475 | 1.00 | 35.04 |
| ATOM | 977 | CG | LYS | 135 | 15.316 | 12.391 | 15.261 | 1.00 | 36.23 |
| ATOM | 978 | CD | LYS | 135 | 15.762 | 12.093 | 13.822 | 1.00 | 36.68 |
| ATOM | 979 | CE | LYS | 135 | 16.943 | 12.925 | 13.375 | 1.00 | 37.17 |
| ATOM | 980 | NZ | LYS | 135 | 17.400 | 12.513 | 12.026 | 1.00 | 37.53 |
| ATOM | 981 | C | LYS | 135 | 13.126 | 13.031 | 17.388 | 1.00 | 34.80 |
| ATOM | 982 | O | LYS | 135 | 13.989 | 13.722 | 17.929 | 1.00 | 35.38 |
| ATOM | 983 | N | ALA | 136 | 11.868 | 13.435 | 17.235 | 1.00 | 33.78 |
| ATOM | 984 | CA | ALA | 136 | 11.413 | 14.764 | 17.631 | 1.00 | 32.23 |
| ATOM | 985 | CB | ALA | 136 | 10.042 | 15.022 | 17.051 | 1.00 | 31.71 |
| ATOM | 986 | C | ALA | 136 | 11.385 | 15.041 | 19.126 | 1.00 | 31.58 |
| ATOM | 987 | O | ALA | 136 | 11.396 | 16.198 | 19.538 | 1.00 | 31.40 |
| ATOM | 988 | N | ILE | 137 | 11.359 | 13.988 | 19.935 | 1.00 | 30.43 |
| ATOM | 989 | CA | ILE | 137 | 11.299 | 14.136 | 21.383 | 1.00 | 29.08 |
| ATOM | 990 | CB | ILE | 137 | 10.505 | 12.971 | 22.010 | 1.00 | 28.62 |
| ATOM | 991 | CG2 | ILE | 137 | 9.097 | 12.878 | 21.396 | 1.00 | 27.72 |
| ATOM | 992 | CG1 | ILE | 137 | 11.263 | 11.660 | 21.786 | 1.00 | 27.64 |
| ATOM | 993 | CD1 | ILE | 137 | 10.712 | 10.490 | 22.574 | 1.00 | 26.85 |
| ATOM | 994 | C | ILE | 137 | 12.663 | 14.167 | 22.063 | 1.00 | 29.05 |
| ATOM | 995 | O | ILE | 137 | 12.760 | 14.447 | 23.255 | 1.00 | 29.51 |
| ATOM | 996 | N | LEU | 138 | 13.714 | 13.884 | 21.306 | 1.00 | 28.48 |
| ATOM | 997 | CA | LEU | 138 | 15.067 | 13.822 | 21.855 | 1.00 | 27.74 |
| ATOM | 998 | CB | LEU | 138 | 15.866 | 12.767 | 21.081 | 1.00 | 25.88 |
| ATOM | 999 | CG | LEU | 138 | 15.234 | 11.370 | 21.120 | 1.00 | 23.44 |
| ATOM | 1000 | CD1 | LEU | 138 | 15.866 | 10.470 | 20.079 | 1.00 | 23.09 |
| ATOM | 1001 | CD2 | LEU | 138 | 15.406 | 10.782 | 22.512 | 1.00 | 21.50 |
| ATOM | 1002 | C | LEU | 138 | 15.822 | 15.143 | 21.871 | 1.00 | 27.94 |
| ATOM | 1003 | O | LEU | 138 | 15.954 | 15.810 | 20.849 | 1.00 | 29.08 |
| ATOM | 1004 | N | PHE | 139 | 16.327 | 15.516 | 23.038 | 1.00 | 27.46 |
| ATOM | 1005 | CA | PHE | 139 | 17.058 | 16.765 | 23.158 | 1.00 | 27.29 |
| ATOM | 1006 | CB | PHE | 139 | 16.253 | 17.778 | 23.973 | 1.00 | 26.26 |
| ATOM | 1007 | CG | PHE | 139 | 14.982 | 18.221 | 23.313 | 1.00 | 24.21 |
| ATOM | 1008 | CD1 | PHE | 139 | 13.839 | 17.441 | 23.387 | 1.00 | 23.67 |
| ATOM | 1009 | CD2 | PHE | 139 | 14.922 | 19.441 | 22.644 | 1.00 | 23.89 |
| ATOM | 1010 | CE1 | PHE | 139 | 12.650 | 17.871 | 22.810 | 1.00 | 23.55 |
| ATOM | 1011 | CE2 | PHE | 139 | 13.741 | 19.884 | 22.062 | 1.00 | 23.26 |
| ATOM | 1012 | CZ | PHE | 139 | 12.602 | 19.100 | 22.146 | 1.00 | 23.71 |
| ATOM | 1013 | C | PHE | 139 | 18.411 | 16.588 | 23.805 | 1.00 | 27.66 |
| ATOM | 1014 | O | PHE | 139 | 18.578 | 15.796 | 24.720 | 1.00 | 27.66 |
| ATOM | 1015 | N | LEU | 140 | 19.380 | 17.345 | 23.320 | 1.00 | 28.50 |
| ATOM | 1016 | CA | LEU | 140 | 20.718 | 17.291 | 23.864 | 1.00 | 29.25 |
| ATOM | 1017 | CB | LEU | 140 | 21.738 | 17.164 | 22.739 | 1.00 | 27.95 |
| ATOM | 1018 | CG | LEU | 140 | 23.173 | 16.905 | 23.187 | 1.00 | 27.42 |
| ATOM | 1019 | CD1 | LEU | 140 | 23.232 | 15.557 | 23.885 | 1.00 | 27.33 |
| ATOM | 1020 | CD2 | LEU | 140 | 24.112 | 16.930 | 21.989 | 1.00 | 27.78 |
| ATOM | 1021 | C | LEU | 140 | 20.960 | 18.584 | 24.628 | 1.00 | 30.43 |
| ATOM | 1022 | O | LEU | 140 | 21.002 | 19.662 | 24.036 | 1.00 | 31.10 |
| ATOM | 1023 | N | PRO | 141 | 21.080 | 18.505 | 25.959 | 1.00 | 30.89 |
| ATOM | 1024 | CD | PRO | 141 | 20.724 | 17.391 | 26.850 | 1.00 | 30.79 |
| ATOM | 1025 | CA | PRO | 141 | 21.324 | 19.725 | 26.725 | 1.00 | 32.44 |
| ATOM | 1026 | CB | PRO | 141 | 21.023 | 19.305 | 28.166 | 1.00 | 31.35 |
| ATOM | 1027 | CG | PRO | 141 | 21.308 | 17.839 | 28.164 | 1.00 | 31.22 |
| ATOM | 1028 | C | PRO | 141 | 22.747 | 20.230 | 26.536 | 1.00 | 34.14 |
| ATOM | 1029 | O | PRO | 141 | 23.707 | 19.464 | 26.572 | 1.00 | 34.05 |
| ATOM | 1030 | N | MET | 142 | 22.872 | 21.529 | 26.320 | 1.00 | 36.45 |
| ATOM | 1031 | CA | MET | 142 | 24.166 | 22.148 | 26.110 | 1.00 | 38.91 |
| ATOM | 1032 | CB | MET | 142 | 24.315 | 22.519 | 24.640 | 1.00 | 37.58 |
| ATOM | 1033 | CG | MET | 142 | 24.203 | 21.341 | 23.701 | 1.00 | 36.89 |
| ATOM | 1034 | SD | MET | 142 | 24.345 | 21.837 | 21.984 | 1.00 | 37.07 |
| ATOM | 1035 | CE | MET | 142 | 26.022 | 22.298 | 21.902 | 1.00 | 37.42 |
| ATOM | 1036 | C | MET | 142 | 24.244 | 23.395 | 26.964 | 1.00 | 41.41 |
| ATOM | 1037 | O | MET | 142 | 23.239 | 24.079 | 27.152 | 1.00 | 41.83 |
| ATOM | 1038 | N | SER | 143 | 25.426 | 23.696 | 27.487 | 1.00 | 44.37 |
| ATOM | 1039 | CA | SER | 143 | 25.576 | 24.883 | 28.313 | 1.00 | 47.76 |
| ATOM | 1040 | CB | SER | 143 | 26.992 | 24.980 | 28.876 | 1.00 | 48.30 |
| ATOM | 1041 | OG | SER | 143 | 27.921 | 25.277 | 27.848 | 1.00 | 49.63 |
| ATOM | 1042 | C | SER | 143 | 25.282 | 26.106 | 27.457 | 1.00 | 49.81 |
| ATOM | 1043 | O | SER | 143 | 25.415 | 26.071 | 26.228 | 1.00 | 50.18 |
| ATOM | 1044 | N | ALA | 144 | 24.866 | 27.184 | 28.105 | 1.00 | 52.23 |
| ATOM | 1045 | CA | ALA | 144 | 24.557 | 28.406 | 27.387 | 1.00 | 55.05 |
| ATOM | 1046 | CB | ALA | 144 | 23.208 | 28.947 | 27.821 | 1.00 | 54.70 |
| ATOM | 1047 | C | ALA | 144 | 25.637 | 29.433 | 27.663 | 1.00 | 57.35 |
| ATOM | 1048 | O | ALA | 144 | 25.737 | 29.956 | 28.780 | 1.00 | 58.14 |
| ATOM | 1049 | N | LYS | 145 | 26.447 | 29.719 | 26.646 | 1.00 | 59.20 |
| ATOM | 1050 | CA | LYS | 145 | 27.511 | 30.705 | 26.772 | 1.00 | 60.85 |
| ATOM | 1051 | CB | LYS | 145 | 28.786 | 30.058 | 27.335 | 1.00 | 61.42 |
| ATOM | 1052 | CG | LYS | 145 | 28.596 | 29.428 | 28.718 | 1.00 | 62.32 |
| ATOM | 1053 | CD | LYS | 145 | 29.701 | 29.806 | 29.691 | 1.00 | 62.53 |
| ATOM | 1054 | CE | LYS | 145 | 29.391 | 29.263 | 31.078 | 1.00 | 63.58 |
| ATOM | 1055 | NZ | LYS | 145 | 30.384 | 29.679 | 32.118 | 1.00 | 64.35 |
| ATOM | 1056 | C | LYS | 145 | 27.780 | 31.309 | 25.401 | 1.00 | 61.86 |
| ATOM | 1057 | O | LYS | 145 | 27.822 | 32.535 | 25.247 | 1.00 | 62.09 |
| ATOM | 1058 | N | ALA | 146 | 27.942 | 30.445 | 24.401 | 1.00 | 62.32 |
| ATOM | 1059 | CA | ALA | 146 | 28.205 | 30.902 | 23.044 | 1.00 | 62.92 |
| ATOM | 1060 | CB | ALA | 146 | 29.371 | 30.123 | 22.453 | 1.00 | 62.85 |
| ATOM | 1061 | C | ALA | 146 | 26.969 | 30.758 | 22.158 | 1.00 | 63.51 |
| ATOM | 1062 | O | ALA | 146 | 26.349 | 31.806 | 21.859 | 1.00 | 63.75 |
| ATOM | 1063 | CB | HIS | 1016 | 35.195 | −13.780 | 34.624 | 1.00 | 50.24 |
| ATOM | 1064 | CG | HIS | 1016 | 36.186 | −13.875 | 35.736 | 1.00 | 52.02 |
| ATOM | 1065 | CD2 | HIS | 1016 | 36.027 | −14.140 | 37.054 | 1.00 | 52.96 |
| ATOM | 1066 | ND1 | HIS | 1016 | 37.539 | −13.702 | 35.540 | 1.00 | 53.04 |
| ATOM | 1067 | CE1 | HIS | 1016 | 38.172 | −13.857 | 36.691 | 1.00 | 53.67 |
| ATOM | 1068 | NE2 | HIS | 1016 | 37.277 | −14.124 | 37.626 | 1.00 | 53.72 |
| ATOM | 1069 | C | HIS | 1016 | 36.657 | −12.614 | 32.965 | 1.00 | 47.80 |
| ATOM | 1070 | O | HIS | 1016 | 36.809 | −13.475 | 32.089 | 1.00 | 46.82 |
| ATOM | 1071 | N | HIS | 1016 | 34.177 | −12.360 | 32.863 | 1.00 | 48.65 |
| ATOM | 1072 | CA | HIS | 1016 | 35.356 | −12.525 | 33.770 | 1.00 | 48.80 |
| ATOM | 1073 | N | PHE | 1017 | 37.594 | −11.723 | 33.286 | 1.00 | 46.61 |
| ATOM | 1074 | CA | PHE | 1017 | 38.873 | −11.638 | 32.591 | 1.00 | 46.23 |
| ATOM | 1075 | CB | PHE | 1017 | 39.765 | −10.581 | 33.262 | 1.00 | 46.95 |
| ATOM | 1076 | CG | PHE | 1017 | 40.338 | −11.019 | 34.573 | 1.00 | 47.43 |
| ATOM | 1077 | CD1 | PHE | 1017 | 41.509 | −11.770 | 34.617 | 1.00 | 48.33 |
| ATOM | 1078 | CD2 | PHE | 1017 | 39.694 | −10.717 | 35.760 | 1.00 | 47.37 |
| ATOM | 1079 | CE1 | PHE | 1017 | 42.030 | −12.218 | 35.828 | 1.00 | 48.64 |
| ATOM | 1080 | CE2 | PHE | 1017 | 40.205 | −11.158 | 36.977 | 1.00 | 48.23 |
| ATOM | 1081 | CZ | PHE | 1017 | 41.374 | −11.910 | 37.010 | 1.00 | 48.83 |
| ATOM | 1082 | C | PHE | 1017 | 39.616 | −12.978 | 32.470 | 1.00 | 45.73 |
| ATOM | 1083 | O | PHE | 1017 | 40.361 | −13.183 | 31.509 | 1.00 | 45.94 |
| ATOM | 1084 | N | LYS | 1018 | 39.409 | −13.889 | 33.423 | 1.00 | 44.76 |
| ATOM | 1085 | CA | LYS | 1018 | 40.072 | −15.197 | 33.385 | 1.00 | 44.28 |
| ATOM | 1086 | CB | LYS | 1018 | 39.821 | −15.964 | 34.691 | 1.00 | 43.48 |
| ATOM | 1087 | C | LYS | 1018 | 39.620 | −16.062 | 32.195 | 1.00 | 44.31 |
| ATOM | 1088 | O | LYS | 1018 | 40.428 | −16.792 | 31.610 | 1.00 | 44.33 |
| ATOM | 1089 | N | ASP | 1019 | 38.335 | −15.972 | 31.843 | 1.00 | 43.68 |
| ATOM | 1090 | CA | ASP | 1019 | 37.775 | −16.751 | 30.743 | 1.00 | 42.46 |
| ATOM | 1091 | CB | ASP | 1019 | 36.260 | −16.563 | 30.652 | 1.00 | 44.06 |
| ATOM | 1092 | CG | ASP | 1019 | 35.526 | −17.094 | 31.869 | 1.00 | 45.48 |
| ATOM | 1093 | OD1 | ASP | 1019 | 35.862 | −18.213 | 32.333 | 1.00 | 46.23 |
| ATOM | 1094 | OD2 | ASP | 1019 | 34.600 | −16.394 | 32.348 | 1.00 | 46.31 |
| ATOM | 1095 | C | ASP | 1019 | 38.377 | −16.407 | 29.393 | 1.00 | 41.43 |
| ATOM | 1096 | O | ASP | 1019 | 38.993 | −15.357 | 29.220 | 1.00 | 41.52 |
| ATOM | 1097 | N | PRO | 1020 | 38.205 | −17.306 | 28.414 | 1.00 | 40.39 |
| ATOM | 1098 | CD | PRO | 1020 | 37.701 | −18.680 | 28.566 | 1.00 | 40.94 |
| ATOM | 1099 | CA | PRO | 1020 | 38.723 | −17.110 | 27.062 | 1.00 | 39.43 |
| ATOM | 1100 | CB | PRO | 1020 | 38.590 | −18.495 | 26.427 | 1.00 | 39.90 |
| ATOM | 1101 | CG | PRO | 1020 | 38.567 | −19.421 | 27.595 | 1.00 | 40.54 |
| ATOM | 1102 | C | PRO | 1020 | 37.839 | −16.097 | 26.366 | 1.00 | 38.51 |
| ATOM | 1103 | O | PRO | 1020 | 36.695 | −15.889 | 26.763 | 1.00 | 38.04 |
| ATOM | 1104 | N | LYS | 1021 | 38.362 | −15.480 | 25.320 | 1.00 | 37.52 |
| ATOM | 1105 | CA | LYS | 1021 | 37.596 | −14.493 | 24.595 | 1.00 | 37.31 |
| ATOM | 1106 | CB | LYS | 1021 | 38.062 | −13.090 | 24.985 | 1.00 | 37.95 |
| ATOM | 1107 | CG | LYS | 1021 | 37.948 | −12.791 | 26.463 | 1.00 | 39.55 |
| ATOM | 1108 | CD | LYS | 1021 | 38.526 | −11.420 | 26.815 | 1.00 | 40.62 |
| ATOM | 1109 | CE | LYS | 1021 | 38.135 | −11.023 | 28.242 | 1.00 | 41.41 |
| ATOM | 1110 | NZ | LYS | 1021 | 38.672 | −9.689 | 28.653 | 1.00 | 42.03 |
| ATOM | 1111 | C | LYS | 1021 | 37.764 | −14.672 | 23.100 | 1.00 | 36.82 |
| ATOM | 1112 | O | LYS | 1021 | 38.705 | −15.313 | 22.649 | 1.00 | 36.62 |
| ATOM | 1113 | N | ARG | 1022 | 36.829 | −14.111 | 22.338 | 1.00 | 36.29 |
| ATOM | 1114 | CA | ARG | 1022 | 36.894 | −14.145 | 20.890 | 1.00 | 35.27 |
| ATOM | 1115 | CB | ARG | 1022 | 35.555 | −14.575 | 20.291 | 1.00 | 36.88 |
| ATOM | 1116 | CG | ARG | 1022 | 35.203 | −16.040 | 20.495 | 1.00 | 39.93 |
| ATOM | 1117 | CD | ARG | 1022 | 34.007 | −16.421 | 19.647 | 1.00 | 42.65 |
| ATOM | 1118 | NE | ARG | 1022 | 32.783 | −16.644 | 20.419 | 1.00 | 45.95 |
| ATOM | 1119 | CZ | ARG | 1022 | 32.443 | −17.805 | 20.977 | 1.00 | 47.03 |
| ATOM | 1120 | NH1 | ARG | 1022 | 33.237 | −18.867 | 20.856 | 1.00 | 47.13 |
| ATOM | 1121 | NH2 | ARG | 1022 | 31.295 | −17.909 | 21.641 | 1.00 | 47.38 |
| ATOM | 1122 | C | ARG | 1022 | 37.173 | −12.702 | 20.509 | 1.00 | 34.79 |
| ATOM | 1123 | O | ARG | 1022 | 36.624 | −11.787 | 21.120 | 1.00 | 35.24 |
| ATOM | 1124 | N | LEU | 1023 | 38.041 | −12.483 | 19.531 | 1.00 | 33.72 |
| ATOM | 1125 | CA | LEU | 1023 | 38.329 | −11.131 | 19.092 | 1.00 | 32.57 |
| ATOM | 1126 | CB | LEU | 1023 | 39.836 | −10.898 | 19.025 | 1.00 | 32.72 |
| ATOM | 1127 | CG | LEU | 1023 | 40.550 | −10.641 | 20.362 | 1.00 | 33.54 |
| ATOM | 1128 | CD1 | LEU | 1023 | 42.045 | −10.526 | 20.128 | 1.00 | 33.26 |
| ATOM | 1129 | CD2 | LEU | 1023 | 40.036 | −9.354 | 21.006 | 1.00 | 33.55 |
| ATOM | 1130 | C | LEU | 1023 | 37.675 | −10.903 | 17.729 | 1.00 | 32.72 |
| ATOM | 1131 | O | LEU | 1023 | 38.129 | −11.411 | 16.703 | 1.00 | 33.04 |
| ATOM | 1132 | N | TYR | 1024 | 36.581 | −10.149 | 17.743 | 1.00 | 32.24 |
| ATOM | 1133 | CA | TYR | 1024 | 35.814 | −9.825 | 16.547 | 1.00 | 31.22 |
| ATOM | 1134 | CB | TYR | 1024 | 34.357 | −9.629 | 16.952 | 1.00 | 30.87 |
| ATOM | 1135 | CG | TYR | 1024 | 33.393 | −9.182 | 15.870 | 1.00 | 30.66 |
| ATOM | 1136 | CD1 | TYR | 1024 | 33.291 | −7.838 | 15.499 | 1.00 | 30.06 |
| ATOM | 1137 | CE1 | TYR | 1024 | 32.321 | −7.415 | 14.583 | 1.00 | 29.62 |
| ATOM | 1138 | CD2 | TYR | 1024 | 32.511 | −10.095 | 15.287 | 1.00 | 30.22 |
| ATOM | 1139 | CE2 | TYR | 1024 | 31.548 | −9.686 | 14.373 | 1.00 | 30.21 |
| ATOM | 1140 | CZ | TYR | 1024 | 31.451 | −8.350 | 14.027 | 1.00 | 30.30 |
| ATOM | 1141 | OH | TYR | 1024 | 30.467 | −7.975 | 13.139 | 1.00 | 29.32 |
| ATOM | 1142 | C | TYR | 1024 | 36.388 | −8.559 | 15.921 | 1.00 | 32.03 |
| ATOM | 1143 | O | TYR | 1024 | 36.392 | −7.487 | 16.535 | 1.00 | 32.58 |
| ATOM | 1144 | N | CYS | 1025 | 36.883 | −8.683 | 14.696 | 1.00 | 32.09 |
| ATOM | 1145 | CA | CYS | 1025 | 37.470 | −7.544 | 14.005 | 1.00 | 32.66 |
| ATOM | 1146 | CB | CYS | 1025 | 38.417 | −8.006 | 12.902 | 1.00 | 32.69 |
| ATOM | 1147 | SG | CYS | 1025 | 39.333 | −6.623 | 12.164 | 1.00 | 33.72 |
| ATOM | 1148 | C | CYS | 1025 | 36.401 | −6.657 | 13.394 | 1.00 | 33.09 |
| ATOM | 1149 | O | CYS | 1025 | 35.480 | −7.144 | 12.743 | 1.00 | 33.19 |
| ATOM | 1150 | N | LYS | 1026 | 36.531 | −5.350 | 13.599 | 1.00 | 33.39 |
| ATOM | 1151 | CA | LYS | 1026 | 35.556 | −4.410 | 13.066 | 1.00 | 33.88 |
| ATOM | 1152 | CB | LYS | 1026 | 35.837 | −2.996 | 13.575 | 1.00 | 32.94 |
| ATOM | 1153 | CG | LYS | 1026 | 34.803 | −1.988 | 13.131 | 1.00 | 32.23 |
| ATOM | 1154 | CD | LYS | 1026 | 35.167 | −0.593 | 13.576 | 1.00 | 33.49 |
| ATOM | 1155 | CE | LYS | 1026 | 34.224 | 0.439 | 12.961 | 1.00 | 34.51 |
| ATOM | 1156 | NZ | LYS | 1026 | 34.556 | 1.836 | 13.386 | 1.00 | 34.04 |
| ATOM | 1157 | C | LYS | 1026 | 35.581 | −4.415 | 11.545 | 1.00 | 34.99 |
| ATOM | 1158 | O | LYS | 1026 | 34.585 | −4.101 | 10.890 | 1.00 | 35.16 |
| ATOM | 1159 | N | ASN | 1027 | 36.722 | −4.794 | 10.986 | 1.00 | 36.46 |
| ATOM | 1160 | CA | ASN | 1027 | 36.892 | −4.819 | 9.533 | 1.00 | 37.34 |
| ATOM | 1161 | CB | ASN | 1027 | 38.373 | −4.627 | 9.194 | 1.00 | 38.46 |
| ATOM | 1162 | CG | ASN | 1027 | 38.617 | −4.451 | 7.708 | 1.00 | 39.76 |
| ATOM | 1163 | OD1 | ASN | 1027 | 37.787 | −3.883 | 6.996 | 1.00 | 40.53 |
| ATOM | 1164 | ND2 | ASN | 1027 | 39.770 | −4.918 | 7.234 | 1.00 | 39.60 |
| ATOM | 1165 | C | ASN | 1027 | 36.366 | −6.094 | 8.872 | 1.00 | 37.34 |
| ATOM | 1166 | O | ASN | 1027 | 37.134 | −7.002 | 8.559 | 1.00 | 37.52 |
| ATOM | 1167 | N | GLY | 1028 | 35.054 | −6.160 | 8.670 | 1.00 | 37.05 |
| ATOM | 1168 | CA | GLY | 1028 | 34.472 | −7.328 | 8.039 | 1.00 | 37.13 |
| ATOM | 1169 | C | GLY | 1028 | 33.801 | −8.297 | 8.996 | 1.00 | 37.68 |
| ATOM | 1170 | O | GLY | 1028 | 33.064 | −9.183 | 8.560 | 1.00 | 38.83 |
| ATOM | 1171 | N | GLY | 1029 | 34.053 | −8.148 | 10.292 | 1.00 | 36.79 |
| ATOM | 1172 | CA | GLY | 1029 | 33.435 | −9.038 | 11.256 | 1.00 | 36.22 |
| ATOM | 1173 | C | GLY | 1029 | 34.072 | −10.410 | 11.348 | 1.00 | 36.20 |
| ATOM | 1174 | O | GLY | 1029 | 33.397 | −11.394 | 11.643 | 1.00 | 36.97 |
| ATOM | 1175 | N | PHE | 1030 | 35.373 | −10.485 | 11.092 | 1.00 | 36.36 |
| ATOM | 1176 | CA | PHE | 1030 | 36.091 | −11.755 | 11.173 | 1.00 | 36.31 |
| ATOM | 1177 | CB | PHE | 1030 | 37.210 | −11.828 | 10.131 | 1.00 | 37.31 |
| ATOM | 1178 | CG | PHE | 1030 | 36.732 | −11.771 | 8.711 | 1.00 | 38.62 |
| ATOM | 1179 | CD1 | PHE | 1030 | 36.850 | −10.598 | 7.971 | 1.00 | 39.50 |
| ATOM | 1180 | CD2 | PHE | 1030 | 36.174 | −12.893 | 8.108 | 1.00 | 39.20 |
| ATOM | 1181 | CE1 | PHE | 1030 | 36.418 | −10.542 | 6.642 | 1.00 | 40.39 |
| ATOM | 1182 | CE2 | PHE | 1030 | 35.739 | −12.850 | 6.786 | 1.00 | 39.85 |
| ATOM | 1183 | CZ | PHE | 1030 | 35.862 | −11.672 | 6.049 | 1.00 | 39.97 |
| ATOM | 1184 | C | PHE | 1030 | 36.722 | −11.925 | 12.548 | 1.00 | 36.02 |
| ATOM | 1185 | O | PHE | 1030 | 37.332 | −10.985 | 13.068 | 1.00 | 35.58 |
| ATOM | 1186 | N | PHE | 1031 | 36.575 | −13.120 | 13.125 | 1.00 | 35.55 |
| ATOM | 1187 | CA | PHE | 1031 | 37.164 | −13.428 | 14.427 | 1.00 | 35.33 |
| ATOM | 1188 | CB | PHE | 1031 | 36.429 | −14.582 | 15.111 | 1.00 | 34.97 |
| ATOM | 1189 | CG | PHE | 1031 | 35.061 | −14.216 | 15.624 | 1.00 | 36.46 |
| ATOM | 1190 | CD1 | PHE | 1031 | 34.918 | −13.387 | 16.732 | 1.00 | 36.87 |
| ATOM | 1191 | CD2 | PHE | 1031 | 33.914 | −14.691 | 14.992 | 1.00 | 36.43 |
| ATOM | 1192 | CE1 | PHE | 1031 | 33.657 | −13.033 | 17.204 | 1.00 | 36.47 |
| ATOM | 1193 | CE2 | PHE | 1031 | 32.652 | −14.346 | 15.455 | 1.00 | 36.37 |
| ATOM | 1194 | CZ | PHE | 1031 | 32.524 | −13.514 | 16.565 | 1.00 | 37.39 |
| ATOM | 1195 | C | PHE | 1031 | 38.617 | −13.828 | 14.214 | 1.00 | 35.67 |
| ATOM | 1196 | O | PHE | 1031 | 38.934 | −14.541 | 13.268 | 1.00 | 36.04 |
| ATOM | 1197 | N | LEU | 1032 | 39.505 | −13.357 | 15.081 | 1.00 | 35.91 |
| ATOM | 1198 | CA | LEU | 1032 | 40.910 | −13.710 | 14.956 | 1.00 | 36.29 |
| ATOM | 1199 | CB | LEU | 1032 | 41.748 | −12.939 | 15.973 | 1.00 | 36.22 |
| ATOM | 1200 | CG | LEU | 1032 | 43.270 | −13.086 | 15.859 | 1.00 | 36.44 |
| ATOM | 1201 | CD1 | LEU | 1032 | 43.746 | −12.542 | 14.506 | 1.00 | 35.45 |
| ATOM | 1202 | CD2 | LEU | 1032 | 43.942 | −12.325 | 17.008 | 1.00 | 35.42 |
| ATOM | 1203 | C | LEU | 1032 | 41.028 | −15.211 | 15.215 | 1.00 | 37.32 |
| ATOM | 1204 | O | LEU | 1032 | 40.528 | −15.721 | 16.228 | 1.00 | 37.07 |
| ATOM | 1205 | N | ARG | 1033 | 41.684 | −15.919 | 14.296 | 1.00 | 38.42 |
| ATOM | 1206 | CA | ARG | 1033 | 41.830 | −17.363 | 14.433 | 1.00 | 39.17 |
| ATOM | 1207 | CB | ARG | 1033 | 41.028 | −18.084 | 13.353 | 1.00 | 40.39 |
| ATOM | 1208 | CG | ARG | 1033 | 41.089 | −19.600 | 13.477 | 1.00 | 41.83 |
| ATOM | 1209 | CD | ARG | 1033 | 40.165 | −20.282 | 12.479 | 1.00 | 41.78 |
| ATOM | 1210 | NE | ARG | 1033 | 40.487 | −19.904 | 11.105 | 1.00 | 41.08 |
| ATOM | 1211 | CZ | ARG | 1033 | 39.854 | −20.376 | 10.038 | 1.00 | 40.78 |
| ATOM | 1212 | NH1 | ARG | 1033 | 38.865 | −21.248 | 10.181 | 1.00 | 40.23 |
| ATOM | 1213 | NH2 | ARG | 1033 | 40.203 | −19.965 | 8.827 | 1.00 | 40.75 |
| ATOM | 1214 | C | ARG | 1033 | 43.262 | −17.856 | 14.403 | 1.00 | 39.62 |
| ATOM | 1215 | O | ARG | 1033 | 44.044 | −17.486 | 13.526 | 1.00 | 39.14 |
| ATOM | 1216 | N | ILE | 1034 | 43.585 | −18.694 | 15.386 | 1.00 | 40.82 |
| ATOM | 1217 | CA | ILE | 1034 | 44.909 | −19.289 | 15.528 | 1.00 | 42.03 |
| ATOM | 1218 | CB | ILE | 1034 | 45.430 | −19.156 | 16.973 | 1.00 | 42.56 |
| ATOM | 1219 | CG2 | ILE | 1034 | 46.857 | −19.674 | 17.054 | 1.00 | 43.23 |
| ATOM | 1220 | CG1 | ILE | 1034 | 45.385 | −17.698 | 17.426 | 1.00 | 42.52 |
| ATOM | 1221 | CD1 | ILE | 1034 | 46.315 | −16.793 | 16.667 | 1.00 | 43.24 |
| ATOM | 1222 | C | ILE | 1034 | 44.784 | −20.783 | 15.211 | 1.00 | 43.26 |
| ATOM | 1223 | O | ILE | 1034 | 44.193 | −21.547 | 15.986 | 1.00 | 42.66 |
| ATOM | 1224 | N | HIS | 1035 | 45.329 | −21.190 | 14.066 | 1.00 | 44.90 |
| ATOM | 1225 | CA | HIS | 1035 | 45.283 | −22.592 | 13.634 | 1.00 | 46.39 |
| ATOM | 1226 | CB | HIS | 1035 | 45.587 | −22.721 | 12.133 | 1.00 | 47.94 |
| ATOM | 1227 | CG | HIS | 1035 | 44.485 | −22.245 | 11.237 | 1.00 | 49.89 |
| ATOM | 1228 | CD2 | HIS | 1035 | 44.422 | −21.196 | 10.381 | 1.00 | 50.45 |
| ATOM | 1229 | ND1 | HIS | 1035 | 43.272 | −22.894 | 11.138 | 1.00 | 50.82 |
| ATOM | 1230 | CE1 | HIS | 1035 | 42.510 | −22.265 | 10.259 | 1.00 | 51.27 |
| ATOM | 1231 | NE2 | HIS | 1035 | 43.184 | −21.231 | 9.785 | 1.00 | 50.87 |
| ATOM | 1232 | C | HIS | 1035 | 46.304 | −23.438 | 14.374 | 1.00 | 46.48 |
| ATOM | 1233 | O | HIS | 1035 | 47.392 | −22.963 | 14.704 | 1.00 | 46.12 |
| ATOM | 1234 | N | PRO | 1036 | 45.969 | −24.714 | 14.621 | 1.00 | 47.37 |
| ATOM | 1235 | CD | PRO | 1036 | 44.692 | −25.373 | 14.280 | 1.00 | 47.36 |
| ATOM | 1236 | CA | PRO | 1036 | 46.867 | −25.640 | 15.323 | 1.00 | 47.64 |
| ATOM | 1237 | CB | PRO | 1036 | 46.126 | −26.968 | 15.234 | 1.00 | 47.39 |
| ATOM | 1238 | CG | PRO | 1036 | 44.678 | −26.543 | 15.227 | 1.00 | 47.88 |
| ATOM | 1239 | C | PRO | 1036 | 48.252 | −25.709 | 14.671 | 1.00 | 48.25 |
| ATOM | 1240 | O | PRO | 1036 | 49.258 | −25.917 | 15.353 | 1.00 | 47.81 |
| ATOM | 1241 | N | ASP | 1037 | 48.304 | −25.517 | 13.358 | 1.00 | 49.15 |
| ATOM | 1242 | CA | ASP | 1037 | 49.573 | −25.582 | 12.653 | 1.00 | 50.93 |
| ATOM | 1243 | CB | ASP | 1037 | 49.343 | −26.090 | 11.229 | 1.00 | 52.26 |
| ATOM | 1244 | CG | ASP | 1037 | 48.621 | −25.085 | 10.361 | 1.00 | 53.34 |
| ATOM | 1245 | OD1 | ASP | 1037 | 49.302 | −24.210 | 9.790 | 1.00 | 53.24 |
| ATOM | 1246 | OD2 | ASP | 1037 | 47.378 | −25.167 | 10.257 | 1.00 | 53.76 |
| ATOM | 1247 | C | ASP | 1037 | 50.358 | −24.262 | 12.629 | 1.00 | 51.64 |
| ATOM | 1248 | O | ASP | 1037 | 51.391 | −24.167 | 11.965 | 1.00 | 51.98 |
| ATOM | 1249 | N | GLY | 1038 | 49.875 | −23.250 | 13.350 | 1.00 | 51.85 |
| ATOM | 1250 | CA | GLY | 1038 | 50.575 | −21.973 | 13.388 | 1.00 | 51.09 |
| ATOM | 1251 | C | GLY | 1038 | 50.070 | −20.895 | 12.439 | 1.00 | 51.25 |
| ATOM | 1252 | O | GLY | 1038 | 50.611 | −19.783 | 12.415 | 1.00 | 51.55 |
| ATOM | 1253 | N | ARG | 1039 | 49.044 | −21.207 | 11.651 | 1.00 | 50.66 |
| ATOM | 1254 | CA | ARG | 1039 | 48.492 | −20.226 | 10.721 | 1.00 | 50.17 |
| ATOM | 1255 | CB | ARG | 1039 | 47.801 | −20.924 | 9.544 | 1.00 | 51.11 |
| ATOM | 1256 | CG | ARG | 1039 | 48.743 | −21.626 | 8.577 | 1.00 | 52.29 |
| ATOM | 1257 | CD | ARG | 1039 | 47.991 | −22.197 | 7.380 | 1.00 | 53.18 |
| ATOM | 1258 | NE | ARG | 1039 | 47.064 | −23.264 | 7.751 | 1.00 | 55.18 |
| ATOM | 1259 | CZ | ARG | 1039 | 45.746 | −23.214 | 7.558 | 1.00 | 55.90 |
| ATOM | 1260 | NH1 | ARG | 1039 | 44.976 | −24.236 | 7.926 | 1.00 | 55.99 |
| ATOM | 1261 | NH2 | ARG | 1039 | 45.198 | −22.140 | 6.999 | 1.00 | 55.48 |
| ATOM | 1262 | C | ARG | 1039 | 47.489 | −19.307 | 11.426 | 1.00 | 49.33 |
| ATOM | 1263 | O | ARG | 1039 | 46.669 | −19.765 | 12.228 | 1.00 | 48.82 |
| ATOM | 1264 | N | VAL | 1040 | 47.566 | −18.011 | 11.136 | 1.00 | 48.09 |
| ATOM | 1265 | CA | VAL | 1040 | 46.640 | −17.055 | 11.724 | 1.00 | 46.86 |
| ATOM | 1266 | CB | VAL | 1040 | 47.358 | −16.051 | 12.673 | 1.00 | 47.67 |
| ATOM | 1267 | CG1 | VAL | 1040 | 48.477 | −15.321 | 11.944 | 1.00 | 47.65 |
| ATOM | 1268 | CG2 | VAL | 1040 | 46.350 | −15.056 | 13.218 | 1.00 | 46.87 |
| ATOM | 1269 | C | VAL | 1040 | 45.865 | −16.299 | 10.642 | 1.00 | 46.15 |
| ATOM | 1270 | O | VAL | 1040 | 46.447 | −15.753 | 9.696 | 1.00 | 45.30 |
| ATOM | 1271 | N | ASP | 1041 | 44.544 | −16.290 | 10.793 | 1.00 | 45.06 |
| ATOM | 1272 | CA | ASP | 1041 | 43.655 | −15.624 | 9.852 | 1.00 | 44.54 |
| ATOM | 1273 | CB | ASP | 1041 | 43.388 | −16.550 | 8.656 | 1.00 | 44.79 |
| ATOM | 1274 | CG | ASP | 1041 | 42.527 | −17.768 | 9.021 | 1.00 | 44.82 |
| ATOM | 1275 | OD1 | ASP | 1041 | 42.206 | −18.562 | 8.111 | 1.00 | 44.40 |
| ATOM | 1276 | OD2 | ASP | 1041 | 42.168 | −17.932 | 10.209 | 1.00 | 44.71 |
| ATOM | 1277 | C | ASP | 1041 | 42.334 | −15.294 | 10.558 | 1.00 | 44.15 |
| ATOM | 1278 | O | ASP | 1041 | 42.247 | −15.347 | 11.786 | 1.00 | 43.82 |
| ATOM | 1279 | N | GLY | 1042 | 41.306 | −14.973 | 9.781 | 1.00 | 43.43 |
| ATOM | 1280 | CA | GLY | 1042 | 40.023 | −14.663 | 10.378 | 1.00 | 43.49 |
| ATOM | 1281 | C | GLY | 1042 | 38.880 | −15.449 | 9.772 | 1.00 | 43.28 |
| ATOM | 1282 | O | GLY | 1042 | 38.967 | −15.894 | 8.635 | 1.00 | 43.80 |
| ATOM | 1283 | N | VAL | 1043 | 37.814 | −15.634 | 10.541 | 1.00 | 43.15 |
| ATOM | 1284 | CA | VAL | 1043 | 36.625 | −16.342 | 10.075 | 1.00 | 43.45 |
| ATOM | 1285 | CB | VAL | 1043 | 36.593 | −17.831 | 10.481 | 1.00 | 43.62 |
| ATOM | 1286 | CG1 | VAL | 1043 | 37.418 | −18.651 | 9.524 | 1.00 | 43.54 |
| ATOM | 1287 | CG2 | VAL | 1043 | 37.073 | −17.990 | 11.916 | 1.00 | 43.37 |
| ATOM | 1288 | C | VAL | 1043 | 35.429 | −15.708 | 10.720 | 1.00 | 43.54 |
| ATOM | 1289 | O | VAL | 1043 | 35.532 | −15.146 | 11.805 | 1.00 | 44.05 |
| ATOM | 1290 | N | ARG | 1044 | 34.281 | −15.823 | 10.072 | 1.00 | 43.96 |
| ATOM | 1291 | CA | ARG | 1044 | 33.071 | −15.235 | 10.619 | 1.00 | 44.60 |
| ATOM | 1292 | CB | ARG | 1044 | 32.193 | −14.678 | 9.496 | 1.00 | 44.49 |
| ATOM | 1293 | CG | ARG | 1044 | 32.848 | −13.546 | 8.742 | 1.00 | 44.72 |
| ATOM | 1294 | CD | ARG | 1044 | 31.915 | −12.976 | 7.707 | 1.00 | 44.98 |
| ATOM | 1295 | NE | ARG | 1044 | 32.536 | −11.873 | 6.989 | 1.00 | 45.86 |
| ATOM | 1296 | CZ | ARG | 1044 | 31.984 | −11.269 | 5.947 | 1.00 | 46.63 |
| ATOM | 1297 | NH1 | ARG | 1044 | 30.802 | −11.673 | 5.512 | 1.00 | 47.95 |
| ATOM | 1298 | NH2 | ARG | 1044 | 32.604 | −10.264 | 5.341 | 1.00 | 46.65 |
| ATOM | 1299 | C | ARG | 1044 | 32.266 | −16.200 | 11.471 | 1.00 | 45.06 |
| ATOM | 1300 | O | ARG | 1044 | 31.508 | −15.770 | 12.335 | 1.00 | 45.16 |
| ATOM | 1301 | N | GLU | 1045 | 32.427 | −17.500 | 11.243 | 1.00 | 46.33 |
| ATOM | 1302 | CA | GLU | 1045 | 31.677 | −18.488 | 12.014 | 1.00 | 47.49 |
| ATOM | 1303 | CB | GLU | 1045 | 31.884 | −19.888 | 11.436 | 1.00 | 48.72 |
| ATOM | 1304 | CG | GLU | 1045 | 31.239 | −20.994 | 12.270 | 1.00 | 51.53 |
| ATOM | 1305 | CD | GLU | 1045 | 29.766 | −20.726 | 12.574 | 1.00 | 53.14 |
| ATOM | 1306 | OE1 | GLU | 1045 | 28.969 | −20.609 | 11.613 | 1.00 | 54.44 |
| ATOM | 1307 | OE2 | GLU | 1045 | 29.402 | −20.631 | 13.772 | 1.00 | 53.22 |
| ATOM | 1308 | C | GLU | 1045 | 32.055 | −18.478 | 13.489 | 1.00 | 47.67 |
| ATOM | 1309 | O | GLU | 1045 | 33.147 | −18.899 | 13.865 | 1.00 | 47.68 |
| ATOM | 1310 | N | LYS | 1046 | 31.129 | −18.009 | 14.318 | 1.00 | 48.39 |
| ATOM | 1311 | CA | LYS | 1046 | 31.327 | −17.911 | 15.764 | 1.00 | 49.33 |
| ATOM | 1312 | CB | LYS | 1046 | 30.084 | −17.273 | 16.405 | 1.00 | 50.47 |
| ATOM | 1313 | CG | LYS | 1046 | 30.281 | −16.763 | 17.833 | 1.00 | 52.53 |
| ATOM | 1314 | CD | LYS | 1046 | 29.060 | −15.968 | 18.317 | 1.00 | 53.87 |
| ATOM | 1315 | CE | LYS | 1046 | 29.321 | −15.271 | 19.665 | 1.00 | 54.84 |
| ATOM | 1316 | NZ | LYS | 1046 | 28.246 | −14.274 | 20.023 | 1.00 | 54.95 |
| ATOM | 1317 | C | LYS | 1046 | 31.623 | −19.250 | 16.443 | 1.00 | 49.41 |
| ATOM | 1318 | O | LYS | 1046 | 32.220 | −19.287 | 17.526 | 1.00 | 49.48 |
| ATOM | 1319 | N | SER | 1047 | 31.219 | −20.347 | 15.807 | 1.00 | 49.69 |
| ATOM | 1320 | CA | SER | 1047 | 31.427 | −21.676 | 16.383 | 1.00 | 49.81 |
| ATOM | 1321 | CB | SER | 1047 | 30.296 | −22.621 | 15.965 | 1.00 | 49.64 |
| ATOM | 1322 | OG | SER | 1047 | 30.348 | −22.881 | 14.575 | 1.00 | 51.18 |
| ATOM | 1323 | C | SER | 1047 | 32.779 | −22.312 | 16.049 | 1.00 | 49.64 |
| ATOM | 1324 | O | SER | 1047 | 33.093 | −23.400 | 16.545 | 1.00 | 49.83 |
| ATOM | 1325 | N | ASP | 1048 | 33.580 | −21.650 | 15.215 | 1.00 | 49.08 |
| ATOM | 1326 | CA | ASP | 1048 | 34.892 | −22.195 | 14.886 | 1.00 | 49.34 |
| ATOM | 1327 | CB | ASP | 1048 | 35.678 | −21.214 | 14.013 | 1.00 | 50.61 |
| ATOM | 1328 | CG | ASP | 1048 | 36.989 | −21.802 | 13.512 | 1.00 | 52.25 |
| ATOM | 1329 | OD1 | ASP | 1048 | 37.182 | −21.870 | 12.274 | 1.00 | 51.73 |
| ATOM | 1330 | OD2 | ASP | 1048 | 37.824 | −22.198 | 14.361 | 1.00 | 53.93 |
| ATOM | 1331 | C | ASP | 1048 | 35.629 | −22.446 | 16.210 | 1.00 | 48.69 |
| ATOM | 1332 | O | ASP | 1048 | 35.611 | −21.608 | 17.112 | 1.00 | 49.14 |
| ATOM | 1333 | N | PRO | 1049 | 36.278 | −23.609 | 16.348 | 1.00 | 48.13 |
| ATOM | 1334 | CD | PRO | 1049 | 36.309 | −24.744 | 15.404 | 1.00 | 47.77 |
| ATOM | 1335 | CA | PRO | 1049 | 37.000 | −23.929 | 17.589 | 1.00 | 47.42 |
| ATOM | 1336 | CB | PRO | 1049 | 37.169 | −25.441 | 17.498 | 1.00 | 47.51 |
| ATOM | 1337 | CG | PRO | 1049 | 37.377 | −25.641 | 16.007 | 1.00 | 47.82 |
| ATOM | 1338 | C | PRO | 1049 | 38.339 | −23.222 | 17.803 | 1.00 | 46.24 |
| ATOM | 1339 | O | PRO | 1049 | 38.826 | −23.136 | 18.931 | 1.00 | 46.26 |
| ATOM | 1340 | N | HIS | 1050 | 38.927 | −22.705 | 16.733 | 1.00 | 45.40 |
| ATOM | 1341 | CA | HIS | 1050 | 40.229 | −22.055 | 16.840 | 1.00 | 44.97 |
| ATOM | 1342 | CB | HIS | 1050 | 41.074 | −22.427 | 15.620 | 1.00 | 47.09 |
| ATOM | 1343 | CG | HIS | 1050 | 41.116 | −23.898 | 15.354 | 1.00 | 48.98 |
| ATOM | 1344 | CD2 | HIS | 1050 | 40.546 | −24.643 | 14.378 | 1.00 | 49.76 |
| ATOM | 1345 | ND1 | HIS | 1050 | 41.725 | −24.790 | 16.212 | 1.00 | 49.94 |
| ATOM | 1346 | CE1 | HIS | 1050 | 41.521 | −26.022 | 15.781 | 1.00 | 50.17 |
| ATOM | 1347 | NE2 | HIS | 1050 | 40.807 | −25.961 | 14.671 | 1.00 | 50.81 |
| ATOM | 1348 | C | HIS | 1050 | 40.210 | −20.533 | 17.007 | 1.00 | 43.71 |
| ATOM | 1349 | O | HIS | 1050 | 41.206 | −19.857 | 16.712 | 1.00 | 42.83 |
| ATOM | 1350 | N | ILE | 1051 | 39.090 | −19.991 | 17.474 | 1.00 | 42.29 |
| ATOM | 1351 | CA | ILE | 1051 | 38.986 | −18.550 | 17.666 | 1.00 | 41.42 |
| ATOM | 1352 | CB | ILE | 1051 | 37.754 | −17.949 | 16.937 | 1.00 | 41.85 |
| ATOM | 1353 | CG2 | ILE | 1051 | 37.869 | −18.163 | 15.432 | 1.00 | 40.96 |
| ATOM | 1354 | CG1 | ILE | 1051 | 36.466 | −18.574 | 17.481 | 1.00 | 41.77 |
| ATOM | 1355 | CD1 | ILE | 1051 | 35.202 | −18.055 | 16.819 | 1.00 | 41.31 |
| ATOM | 1356 | C | ILE | 1051 | 38.887 | −18.213 | 19.147 | 1.00 | 41.39 |
| ATOM | 1357 | O | ILE | 1051 | 38.915 | −17.041 | 19.514 | 1.00 | 41.97 |
| ATOM | 1358 | N | LYS | 1052 | 38.751 | −19.235 | 19.990 | 1.00 | 40.65 |
| ATOM | 1359 | CA | LYS | 1052 | 38.691 | −19.019 | 21.430 | 1.00 | 40.17 |
| ATOM | 1360 | CB | LYS | 1052 | 38.097 | −20.240 | 22.138 | 1.00 | 41.55 |
| ATOM | 1361 | CG | LYS | 1052 | 36.582 | −20.393 | 21.930 | 1.00 | 42.42 |
| ATOM | 1362 | CD | LYS | 1052 | 36.033 | −21.605 | 22.674 | 1.00 | 43.81 |
| ATOM | 1363 | CE | LYS | 1052 | 34.504 | −21.708 | 22.575 | 1.00 | 45.45 |
| ATOM | 1364 | NZ | LYS | 1052 | 33.772 | −20.686 | 23.424 | 1.00 | 45.62 |
| ATOM | 1365 | C | LYS | 1052 | 40.126 | −18.757 | 21.878 | 1.00 | 39.41 |
| ATOM | 1366 | O | LYS | 1052 | 40.984 | −19.634 | 21.813 | 1.00 | 39.71 |
| ATOM | 1367 | N | LEU | 1053 | 40.376 | −17.528 | 22.314 | 1.00 | 38.15 |
| ATOM | 1368 | CA | LEU | 1053 | 41.702 | −17.114 | 22.724 | 1.00 | 36.95 |
| ATOM | 1369 | CB | LEU | 1053 | 42.109 | −15.858 | 21.959 | 1.00 | 37.91 |
| ATOM | 1370 | CG | LEU | 1053 | 41.812 | −15.861 | 20.460 | 1.00 | 39.28 |
| ATOM | 1371 | CD1 | LEU | 1053 | 42.326 | −14.563 | 19.826 | 1.00 | 38.72 |
| ATOM | 1372 | CD2 | LEU | 1053 | 42.459 | −17.085 | 19.818 | 1.00 | 39.26 |
| ATOM | 1373 | C | LEU | 1053 | 41.802 | −16.830 | 24.208 | 1.00 | 36.68 |
| ATOM | 1374 | O | LEU | 1053 | 40.811 | −16.526 | 24.862 | 1.00 | 36.32 |
| ATOM | 1375 | N | GLN | 1054 | 43.017 | −16.921 | 24.733 | 1.00 | 36.39 |
| ATOM | 1376 | CA | GLN | 1054 | 43.258 | −16.656 | 26.136 | 1.00 | 36.19 |
| ATOM | 1377 | CB | GLN | 1054 | 43.895 | −17.867 | 26.813 | 1.00 | 37.63 |
| ATOM | 1378 | CG | GLN | 1054 | 44.088 | −17.727 | 28.307 | 1.00 | 40.04 |
| ATOM | 1379 | CD | GLN | 1054 | 42.773 | −17.716 | 29.071 | 1.00 | 41.94 |
| ATOM | 1380 | OE1 | GLN | 1054 | 41.855 | −18.480 | 28.757 | 1.00 | 43.62 |
| ATOM | 1381 | NE2 | GLN | 1054 | 42.679 | −16.862 | 30.093 | 1.00 | 41.66 |
| ATOM | 1382 | C | GLN | 1054 | 44.203 | −15.473 | 26.200 | 1.00 | 35.81 |
| ATOM | 1383 | O | GLN | 1054 | 45.385 | −15.591 | 25.876 | 1.00 | 36.66 |
| ATOM | 1384 | N | LEU | 1055 | 43.667 | −14.324 | 26.599 | 1.00 | 34.90 |
| ATOM | 1385 | CA | LEU | 1055 | 44.448 | −13.100 | 26.715 | 1.00 | 33.68 |
| ATOM | 1386 | CB | LEU | 1055 | 43.544 | −11.889 | 26.475 | 1.00 | 33.52 |
| ATOM | 1387 | CG | LEU | 1055 | 42.994 | −11.606 | 25.075 | 1.00 | 33.79 |
| ATOM | 1388 | CD1 | LEU | 1055 | 44.017 | −10.825 | 24.288 | 1.00 | 34.64 |
| ATOM | 1389 | CD2 | LEU | 1055 | 42.632 | −12.892 | 24.362 | 1.00 | 33.74 |
| ATOM | 1390 | C | LEU | 1055 | 45.001 | −13.054 | 28.129 | 1.00 | 33.26 |
| ATOM | 1391 | O | LEU | 1055 | 44.242 | −13.065 | 29.103 | 1.00 | 33.28 |
| ATOM | 1392 | N | GLN | 1056 | 46.320 | −13.014 | 28.246 | 1.00 | 32.80 |
| ATOM | 1393 | CA | GLN | 1056 | 46.961 | −12.971 | 29.555 | 1.00 | 32.13 |
| ATOM | 1394 | CB | GLN | 1056 | 47.734 | −14.266 | 29.811 | 1.00 | 31.10 |
| ATOM | 1395 | CG | GLN | 1056 | 48.604 | −14.275 | 31.062 | 1.00 | 29.05 |
| ATOM | 1396 | CD | GLN | 1056 | 47.804 | −14.189 | 32.353 | 1.00 | 28.30 |
| ATOM | 1397 | OE1 | GLN | 1056 | 46.848 | −14.936 | 32.564 | 1.00 | 26.33 |
| ATOM | 1398 | NE2 | GLN | 1056 | 48.204 | −13.277 | 33.231 | 1.00 | 28.66 |
| ATOM | 1399 | C | GLN | 1056 | 47.907 | −11.793 | 29.659 | 1.00 | 32.97 |
| ATOM | 1400 | O | GLN | 1056 | 48.793 | −11.609 | 28.827 | 1.00 | 33.80 |
| ATOM | 1401 | N | ALA | 1057 | 47.714 | −10.983 | 30.690 | 1.00 | 34.16 |
| ATOM | 1402 | CA | ALA | 1057 | 48.571 | −9.829 | 30.901 | 1.00 | 34.77 |
| ATOM | 1403 | CB | ALA | 1057 | 47.937 | −8.889 | 31.903 | 1.00 | 35.14 |
| ATOM | 1404 | C | ALA | 1057 | 49.908 | −10.314 | 31.421 | 1.00 | 35.94 |
| ATOM | 1405 | O | ALA | 1057 | 49.965 | −11.185 | 32.289 | 1.00 | 37.40 |
| ATOM | 1406 | N | GLU | 1058 | 50.986 | −9.767 | 30.878 | 1.00 | 36.68 |
| ATOM | 1407 | CA | GLU | 1058 | 52.327 | −10.142 | 31.316 | 1.00 | 38.07 |
| ATOM | 1408 | CB | GLU | 1058 | 53.267 | −10.216 | 30.106 | 1.00 | 40.22 |
| ATOM | 1409 | CG | GLU | 1058 | 54.651 | −10.805 | 30.382 | 1.00 | 42.31 |
| ATOM | 1410 | CD | GLU | 1058 | 54.608 | −12.251 | 30.876 | 1.00 | 43.56 |
| ATOM | 1411 | OE1 | GLU | 1058 | 53.775 | −13.040 | 30.381 | 1.00 | 42.97 |
| ATOM | 1412 | OE2 | GLU | 1058 | 55.427 | −12.603 | 31.755 | 1.00 | 45.41 |
| ATOM | 1413 | C | GLU | 1058 | 52.768 | −9.048 | 32.281 | 1.00 | 37.80 |
| ATOM | 1414 | O | GLU | 1058 | 53.576 | −9.276 | 33.181 | 1.00 | 38.46 |
| ATOM | 1415 | N | GLU | 1059 | 52.209 | −7.860 | 32.072 | 1.00 | 37.53 |
| ATOM | 1416 | CA | GLU | 1059 | 52.463 | −6.681 | 32.890 | 1.00 | 37.34 |
| ATOM | 1417 | CB | GLU | 1059 | 53.814 | −6.051 | 32.543 | 1.00 | 38.89 |
| ATOM | 1418 | CG | GLU | 1059 | 54.015 | −5.731 | 31.068 | 1.00 | 40.83 |
| ATOM | 1419 | CD | GLU | 1059 | 55.296 | −4.945 | 30.813 | 1.00 | 42.41 |
| ATOM | 1420 | OE1 | GLU | 1059 | 56.238 | −5.065 | 31.626 | 1.00 | 42.54 |
| ATOM | 1421 | OE2 | GLU | 1059 | 55.376 | −4.212 | 29.797 | 1.00 | 44.23 |
| ATOM | 1422 | C | GLU | 1059 | 51.341 | −5.710 | 32.556 | 1.00 | 36.49 |
| ATOM | 1423 | O | GLU | 1059 | 50.520 | −5.993 | 31.695 | 1.00 | 36.73 |
| ATOM | 1424 | N | ARG | 1060 | 51.307 | −4.562 | 33.217 | 1.00 | 35.77 |
| ATOM | 1425 | CA | ARG | 1060 | 50.252 | −3.598 | 32.958 | 1.00 | 34.88 |
| ATOM | 1426 | CB | ARG | 1060 | 50.463 | −2.332 | 33.787 | 1.00 | 34.85 |
| ATOM | 1427 | CG | ARG | 1060 | 49.178 | −1.556 | 33.991 | 1.00 | 35.40 |
| ATOM | 1428 | CD | ARG | 1060 | 49.255 | −0.704 | 35.237 | 1.00 | 37.27 |
| ATOM | 1429 | NE | ARG | 1060 | 50.018 | 0.522 | 35.025 | 1.00 | 38.24 |
| ATOM | 1430 | CZ | ARG | 1060 | 49.482 | 1.682 | 34.663 | 1.00 | 38.43 |
| ATOM | 1431 | NH1 | ARG | 1060 | 48.168 | 1.791 | 34.473 | 1.00 | 38.15 |
| ATOM | 1432 | NH2 | ARG | 1060 | 50.264 | 2.734 | 34.481 | 1.00 | 38.56 |
| ATOM | 1433 | C | ARG | 1060 | 50.120 | −3.214 | 31.491 | 1.00 | 34.18 |
| ATOM | 1434 | O | ARG | 1060 | 51.071 | −2.734 | 30.872 | 1.00 | 34.49 |
| ATOM | 1435 | N | GLY | 1061 | 48.928 | −3.430 | 30.944 | 1.00 | 33.22 |
| ATOM | 1436 | CA | GLY | 1061 | 48.664 | −3.075 | 29.562 | 1.00 | 32.26 |
| ATOM | 1437 | C | GLY | 1061 | 49.307 | −3.930 | 28.491 | 1.00 | 31.49 |
| ATOM | 1438 | O | GLY | 1061 | 49.110 | −3.680 | 27.303 | 1.00 | 31.17 |
| ATOM | 1439 | N | VAL | 1062 | 50.075 | −4.938 | 28.892 | 1.00 | 31.31 |
| ATOM | 1440 | CA | VAL | 1062 | 50.734 | −5.806 | 27.925 | 1.00 | 30.67 |
| ATOM | 1441 | CB | VAL | 1062 | 52.269 | −5.783 | 28.118 | 1.00 | 31.35 |
| ATOM | 1442 | CG1 | VAL | 1062 | 52.949 | −6.589 | 27.022 | 1.00 | 31.23 |
| ATOM | 1443 | CG2 | VAL | 1062 | 52.775 | −4.351 | 28.102 | 1.00 | 29.96 |
| ATOM | 1444 | C | VAL | 1062 | 50.215 | −7.225 | 28.056 | 1.00 | 30.42 |
| ATOM | 1445 | O | VAL | 1062 | 50.217 | −7.801 | 29.148 | 1.00 | 31.05 |
| ATOM | 1446 | N | VAL | 1063 | 49.771 | −7.792 | 26.942 | 1.00 | 30.01 |
| ATOM | 1447 | CA | VAL | 1063 | 49.229 | −9.142 | 26.974 | 1.00 | 30.55 |
| ATOM | 1448 | CB | VAL | 1063 | 47.719 | −9.100 | 26.749 | 1.00 | 31.02 |
| ATOM | 1449 | CG1 | VAL | 1063 | 47.068 | −8.189 | 27.768 | 1.00 | 30.19 |
| ATOM | 1450 | CG2 | VAL | 1063 | 47.441 | −8.617 | 25.332 | 1.00 | 30.82 |
| ATOM | 1451 | C | VAL | 1063 | 49.816 | −10.079 | 25.920 | 1.00 | 30.85 |
| ATOM | 1452 | O | VAL | 1063 | 50.499 | −9.642 | 24.980 | 1.00 | 30.00 |
| ATOM | 1453 | N | SER | 1064 | 49.533 | −11.370 | 26.094 | 1.00 | 30.84 |
| ATOM | 1454 | CA | SER | 1064 | 49.941 | −12.395 | 25.146 | 1.00 | 31.36 |
| ATOM | 1455 | CB | SER | 1064 | 50.774 | −13.484 | 25.823 | 1.00 | 32.07 |
| ATOM | 1456 | OG | SER | 1064 | 49.961 | −14.377 | 26.569 | 1.00 | 33.57 |
| ATOM | 1457 | C | SER | 1064 | 48.597 | −12.961 | 24.697 | 1.00 | 31.87 |
| ATOM | 1458 | O | SER | 1064 | 47.622 | −12.945 | 25.463 | 1.00 | 32.25 |
| ATOM | 1459 | N | ILE | 1065 | 48.532 | −13.443 | 23.461 | 1.00 | 32.46 |
| ATOM | 1460 | CA | ILE | 1065 | 47.293 | −13.988 | 22.933 | 1.00 | 32.51 |
| ATOM | 1461 | CB | ILE | 1065 | 46.847 | −13.200 | 21.701 | 1.00 | 32.07 |
| ATOM | 1462 | CG2 | ILE | 1065 | 45.544 | −13.762 | 21.156 | 1.00 | 31.65 |
| ATOM | 1463 | CG1 | ILE | 1065 | 46.668 | −11.729 | 22.076 | 1.00 | 31.58 |
| ATOM | 1464 | CD1 | ILE | 1065 | 46.486 | −10.827 | 20.896 | 1.00 | 30.40 |
| ATOM | 1465 | C | ILE | 1065 | 47.509 | −15.432 | 22.546 | 1.00 | 34.10 |
| ATOM | 1466 | O | ILE | 1065 | 48.212 | −15.725 | 21.579 | 1.00 | 35.54 |
| ATOM | 1467 | N | LYS | 1066 | 46.899 | −16.337 | 23.299 | 1.00 | 34.71 |
| ATOM | 1468 | CA | LYS | 1066 | 47.043 | −17.761 | 23.036 | 1.00 | 35.91 |
| ATOM | 1469 | CB | LYS | 1066 | 47.462 | −18.486 | 24.321 | 1.00 | 38.31 |
| ATOM | 1470 | CG | LYS | 1066 | 47.350 | −20.013 | 24.244 | 1.00 | 40.88 |
| ATOM | 1471 | CD | LYS | 1066 | 47.619 | −20.667 | 25.595 | 1.00 | 42.92 |
| ATOM | 1472 | CE | LYS | 1066 | 47.358 | −22.171 | 25.544 | 1.00 | 44.95 |
| ATOM | 1473 | NZ | LYS | 1066 | 47.701 | −22.842 | 26.833 | 1.00 | 46.13 |
| ATOM | 1474 | C | LYS | 1066 | 45.786 | −18.434 | 22.483 | 1.00 | 35.92 |
| ATOM | 1475 | O | LYS | 1066 | 44.706 | −18.324 | 23.066 | 1.00 | 34.93 |
| ATOM | 1476 | N | GLY | 1067 | 45.940 | −19.141 | 21.363 | 1.00 | 36.18 |
| ATOM | 1477 | CA | GLY | 1067 | 44.820 | −19.854 | 20.776 | 1.00 | 37.17 |
| ATOM | 1478 | C | GLY | 1067 | 44.667 | −21.120 | 21.594 | 1.00 | 37.98 |
| ATOM | 1479 | O | GLY | 1067 | 45.523 | −21.995 | 21.532 | 1.00 | 38.92 |
| ATOM | 1480 | N | VAL | 1068 | 43.591 | −21.223 | 22.364 | 1.00 | 38.49 |
| ATOM | 1481 | CA | VAL | 1068 | 43.381 | −22.379 | 23.225 | 1.00 | 39.65 |
| ATOM | 1482 | CB | VAL | 1068 | 41.994 | −22.329 | 23.882 | 1.00 | 40.03 |
| ATOM | 1483 | CG1 | VAL | 1068 | 41.770 | −23.568 | 24.731 | 1.00 | 39.36 |
| ATOM | 1484 | CG2 | VAL | 1068 | 41.884 | −21.075 | 24.742 | 1.00 | 41.04 |
| ATOM | 1485 | C | VAL | 1068 | 43.570 | −23.748 | 22.572 | 1.00 | 40.97 |
| ATOM | 1486 | O | VAL | 1068 | 44.411 | −24.535 | 23.012 | 1.00 | 41.24 |
| ATOM | 1487 | N | SER | 1069 | 42.799 | −24.041 | 21.531 | 1.00 | 41.94 |
| ATOM | 1488 | CA | SER | 1069 | 42.910 | −25.331 | 20.863 | 1.00 | 42.83 |
| ATOM | 1489 | CB | SER | 1069 | 41.784 | −25.486 | 19.851 | 1.00 | 43.86 |
| ATOM | 1490 | OG | SER | 1069 | 42.018 | −24.631 | 18.746 | 1.00 | 46.24 |
| ATOM | 1491 | C | SER | 1069 | 44.244 | −25.503 | 20.137 | 1.00 | 42.83 |
| ATOM | 1492 | O | SER | 1069 | 44.805 | −26.599 | 20.104 | 1.00 | 43.52 |
| ATOM | 1493 | N | ALA | 1070 | 44.759 | −24.435 | 19.546 | 1.00 | 42.82 |
| ATOM | 1494 | CA | ALA | 1070 | 46.025 | −24.549 | 18.820 | 1.00 | 43.01 |
| ATOM | 1495 | CB | ALA | 1070 | 46.197 | −23.368 | 17.861 | 1.00 | 43.29 |
| ATOM | 1496 | C | ALA | 1070 | 47.216 | −24.624 | 19.766 | 1.00 | 43.23 |
| ATOM | 1497 | O | ALA | 1070 | 48.312 | −25.038 | 19.373 | 1.00 | 43.44 |
| ATOM | 1498 | N | ASN | 1071 | 46.988 | −24.231 | 21.016 | 1.00 | 43.29 |
| ATOM | 1499 | CA | ASN | 1071 | 48.027 | −24.210 | 22.038 | 1.00 | 43.23 |
| ATOM | 1500 | CB | ASN | 1071 | 48.426 | −25.622 | 22.462 | 1.00 | 44.34 |
| ATOM | 1501 | CG | ASN | 1071 | 49.064 | −25.646 | 23.840 | 1.00 | 45.34 |
| ATOM | 1502 | OD1 | ASN | 1071 | 49.794 | −24.723 | 24.212 | 1.00 | 45.90 |
| ATOM | 1503 | ND2 | ASN | 1071 | 48.797 | −26.702 | 24.603 | 1.00 | 45.33 |
| ATOM | 1504 | C | ASN | 1071 | 49.259 | −23.456 | 21.549 | 1.00 | 43.02 |
| ATOM | 1505 | O | ASN | 1071 | 50.394 | −23.836 | 21.838 | 1.00 | 43.64 |
| ATOM | 1506 | N | ARG | 1072 | 49.026 | −22.381 | 20.801 | 1.00 | 43.48 |
| ATOM | 1507 | CA | ARG | 1072 | 50.110 | −21.542 | 20.288 | 1.00 | 43.30 |
| ATOM | 1508 | CB | ARG | 1072 | 50.241 | −21.703 | 18.777 | 1.00 | 44.71 |
| ATOM | 1509 | CG | ARG | 1072 | 50.554 | −23.122 | 18.350 | 1.00 | 46.34 |
| ATOM | 1510 | CD | ARG | 1072 | 51.225 | −23.114 | 16.995 | 1.00 | 47.85 |
| ATOM | 1511 | NE | ARG | 1072 | 51.842 | −24.395 | 16.674 | 1.00 | 48.69 |
| ATOM | 1512 | CZ | ARG | 1072 | 52.787 | −24.550 | 15.755 | 1.00 | 49.14 |
| ATOM | 1513 | NH1 | ARG | 1072 | 53.219 | −23.499 | 15.070 | 1.00 | 49.08 |
| ATOM | 1514 | NH2 | ARG | 1072 | 53.302 | −25.751 | 15.524 | 1.00 | 49.78 |
| ATOM | 1515 | C | ARG | 1072 | 49.852 | −20.075 | 20.626 | 1.00 | 42.76 |
| ATOM | 1516 | O | ARG | 1072 | 48.705 | −19.669 | 20.815 | 1.00 | 42.80 |
| ATOM | 1517 | N | TYR | 1073 | 50.924 | −19.292 | 20.696 | 1.00 | 41.88 |
| ATOM | 1518 | CA | TYR | 1073 | 50.834 | −17.875 | 21.026 | 1.00 | 41.32 |
| ATOM | 1519 | CB | TYR | 1073 | 51.920 | −17.519 | 22.055 | 1.00 | 42.03 |
| ATOM | 1520 | CG | TYR | 1073 | 51.773 | −18.283 | 23.356 | 1.00 | 43.12 |
| ATOM | 1521 | CD1 | TYR | 1073 | 51.810 | −19.679 | 23.374 | 1.00 | 44.00 |
| ATOM | 1522 | CE1 | TYR | 1073 | 51.599 | −20.396 | 24.551 | 1.00 | 43.97 |
| ATOM | 1523 | CD2 | TYR | 1073 | 51.528 | −17.619 | 24.559 | 1.00 | 43.01 |
| ATOM | 1524 | CE2 | TYR | 1073 | 51.316 | −18.326 | 25.740 | 1.00 | 43.33 |
| ATOM | 1525 | CZ | TYR | 1073 | 51.350 | −19.716 | 25.727 | 1.00 | 44.01 |
| ATOM | 1526 | OH | TYR | 1073 | 51.108 | −20.437 | 26.881 | 1.00 | 44.55 |
| ATOM | 1527 | C | TYR | 1073 | 50.992 | −17.014 | 19.785 | 1.00 | 40.80 |
| ATOM | 1528 | O | TYR | 1073 | 51.854 | −17.269 | 18.956 | 1.00 | 41.47 |
| ATOM | 1529 | N | LEU | 1074 | 50.161 | −15.989 | 19.659 | 1.00 | 40.28 |
| ATOM | 1530 | CA | LEU | 1074 | 50.242 | −15.098 | 18.511 | 1.00 | 40.12 |
| ATOM | 1531 | CB | LEU | 1074 | 49.063 | −14.129 | 18.510 | 1.00 | 41.27 |
| ATOM | 1532 | CG | LEU | 1074 | 48.906 | −13.214 | 17.295 | 1.00 | 41.51 |
| ATOM | 1533 | CD1 | LEU | 1074 | 48.573 | −14.034 | 16.037 | 1.00 | 41.06 |
| ATOM | 1534 | CD2 | LEU | 1074 | 47.805 | −12.216 | 17.595 | 1.00 | 41.89 |
| ATOM | 1535 | C | LEU | 1074 | 51.538 | −14.308 | 18.586 | 1.00 | 39.73 |
| ATOM | 1536 | O | LEU | 1074 | 51.931 | −13.842 | 19.657 | 1.00 | 39.05 |
| ATOM | 1537 | N | ALA | 1075 | 52.199 | −14.164 | 17.444 | 1.00 | 40.38 |
| ATOM | 1538 | CA | ALA | 1075 | 53.452 | −13.428 | 17.382 | 1.00 | 41.01 |
| ATOM | 1539 | CB | ALA | 1075 | 54.628 | −14.384 | 17.530 | 1.00 | 41.24 |
| ATOM | 1540 | C | ALA | 1075 | 53.538 | −12.687 | 16.062 | 1.00 | 41.68 |
| ATOM | 1541 | O | ALA | 1075 | 53.034 | −13.164 | 15.044 | 1.00 | 42.01 |
| ATOM | 1542 | N | MET | 1076 | 54.147 | −11.505 | 16.090 | 1.00 | 42.93 |
| ATOM | 1543 | CA | MET | 1076 | 54.307 | −10.719 | 14.876 | 1.00 | 44.36 |
| ATOM | 1544 | CB | MET | 1076 | 53.730 | −9.310 | 15.028 | 1.00 | 45.67 |
| ATOM | 1545 | CG | MET | 1076 | 53.602 | −8.614 | 13.679 | 1.00 | 47.95 |
| ATOM | 1546 | SD | MET | 1076 | 52.884 | −6.975 | 13.725 | 1.00 | 50.84 |
| ATOM | 1547 | CE | MET | 1076 | 54.316 | −6.072 | 14.291 | 1.00 | 50.93 |
| ATOM | 1548 | C | MET | 1076 | 55.792 | −10.640 | 14.575 | 1.00 | 45.02 |
| ATOM | 1549 | O | MET | 1076 | 56.598 | −10.333 | 15.457 | 1.00 | 44.82 |
| ATOM | 1550 | N | LYS | 1077 | 56.150 | −10.912 | 13.324 | 1.00 | 45.89 |
| ATOM | 1551 | CA | LYS | 1077 | 57.553 | −10.913 | 12.927 | 1.00 | 47.40 |
| ATOM | 1552 | CB | LYS | 1077 | 57.768 | −11.892 | 11.768 | 1.00 | 47.77 |
| ATOM | 1553 | CG | LYS | 1077 | 57.055 | −13.225 | 11.941 | 1.00 | 48.14 |
| ATOM | 1554 | CD | LYS | 1077 | 57.434 | −13.921 | 13.237 | 1.00 | 49.22 |
| ATOM | 1555 | CE | LYS | 1077 | 58.912 | −14.257 | 13.289 | 1.00 | 49.55 |
| ATOM | 1556 | NZ | LYS | 1077 | 59.222 | −15.142 | 14.452 | 1.00 | 50.27 |
| ATOM | 1557 | C | LYS | 1077 | 58.101 | −9.548 | 12.537 | 1.00 | 48.26 |
| ATOM | 1558 | O | LYS | 1077 | 57.370 | −8.560 | 12.471 | 1.00 | 48.92 |
| ATOM | 1559 | N | GLU | 1078 | 59.403 | −9.520 | 12.277 | 1.00 | 49.19 |
| ATOM | 1560 | CA | GLU | 1078 | 60.126 | −8.316 | 11.886 | 1.00 | 49.91 |
| ATOM | 1561 | CB | GLU | 1078 | 61.597 | −8.668 | 11.706 | 1.00 | 52.16 |
| ATOM | 1562 | CG | GLU | 1078 | 61.794 | −9.721 | 10.619 | 1.00 | 54.90 |
| ATOM | 1563 | CD | GLU | 1078 | 63.069 | −10.508 | 10.790 | 1.00 | 56.71 |
| ATOM | 1564 | OE1 | GLU | 1078 | 64.153 | −9.896 | 10.674 | 1.00 | 58.13 |
| ATOM | 1565 | OE2 | GLU | 1078 | 62.983 | −11.736 | 11.043 | 1.00 | 57.52 |
| ATOM | 1566 | C | GLU | 1078 | 59.602 | −7.713 | 10.585 | 1.00 | 49.66 |
| ATOM | 1567 | O | GLU | 1078 | 59.733 | −6.507 | 10.359 | 1.00 | 50.24 |
| ATOM | 1568 | N | ASP | 1079 | 59.036 | −8.548 | 9.717 | 1.00 | 49.12 |
| ATOM | 1569 | CA | ASP | 1079 | 58.511 | −8.054 | 8.441 | 1.00 | 49.19 |
| ATOM | 1570 | CB | ASP | 1079 | 58.653 | −9.120 | 7.342 | 1.00 | 50.24 |
| ATOM | 1571 | CG | ASP | 1079 | 57.938 | −10.417 | 7.684 | 1.00 | 52.00 |
| ATOM | 1572 | OD1 | ASP | 1079 | 57.965 | −11.349 | 6.842 | 1.00 | 52.09 |
| ATOM | 1573 | OD2 | ASP | 1079 | 57.350 | −10.506 | 8.793 | 1.00 | 52.96 |
| ATOM | 1574 | C | ASP | 1079 | 57.047 | −7.630 | 8.582 | 1.00 | 48.50 |
| ATOM | 1575 | O | ASP | 1079 | 56.482 | −6.988 | 7.688 | 1.00 | 48.36 |
| ATOM | 1576 | N | GLY | 1080 | 56.443 | −7.991 | 9.715 | 1.00 | 47.52 |
| ATOM | 1577 | CA | GLY | 1080 | 55.061 | −7.631 | 9.971 | 1.00 | 46.23 |
| ATOM | 1578 | C | GLY | 1080 | 54.064 | −8.759 | 9.811 | 1.00 | 45.48 |
| ATOM | 1579 | O | GLY | 1080 | 52.879 | −8.567 | 10.069 | 1.00 | 45.56 |
| ATOM | 1580 | N | ARG | 1081 | 54.522 | −9.933 | 9.391 | 1.00 | 44.96 |
| ATOM | 1581 | CA | ARG | 1081 | 53.607 | −11.052 | 9.213 | 1.00 | 44.59 |
| ATOM | 1582 | CB | ARG | 1081 | 54.221 | −12.115 | 8.286 | 1.00 | 46.85 |
| ATOM | 1583 | CG | ARG | 1081 | 55.528 | −12.734 | 8.752 | 1.00 | 48.82 |
| ATOM | 1584 | CD | ARG | 1081 | 56.105 | −13.672 | 7.679 | 1.00 | 51.03 |
| ATOM | 1585 | NE | ARG | 1081 | 57.115 | −14.587 | 8.218 | 1.00 | 52.75 |
| ATOM | 1586 | CZ | ARG | 1081 | 58.347 | −14.233 | 8.581 | 1.00 | 53.55 |
| ATOM | 1587 | NH1 | ARG | 1081 | 58.745 | −12.975 | 8.461 | 1.00 | 53.83 |
| ATOM | 1588 | NH2 | ARG | 1081 | 59.183 | −15.139 | 9.079 | 1.00 | 54.39 |
| ATOM | 1589 | C | ARG | 1081 | 53.240 | −11.649 | 10.558 | 1.00 | 43.71 |
| ATOM | 1590 | O | ARG | 1081 | 53.967 | −11.475 | 11.537 | 1.00 | 43.67 |
| ATOM | 1591 | N | LEU | 1082 | 52.103 | −12.339 | 10.610 | 1.00 | 43.10 |
| ATOM | 1592 | CA | LEU | 1082 | 51.630 | −12.940 | 11.852 | 1.00 | 41.76 |
| ATOM | 1593 | CB | LEU | 1082 | 50.189 | −12.504 | 12.142 | 1.00 | 41.58 |
| ATOM | 1594 | CG | LEU | 1082 | 49.857 | −11.017 | 12.311 | 1.00 | 40.60 |
| ATOM | 1595 | CD1 | LEU | 1082 | 48.382 | −10.887 | 12.651 | 1.00 | 39.79 |
| ATOM | 1596 | CD2 | LEU | 1082 | 50.713 | −10.392 | 13.401 | 1.00 | 39.86 |
| ATOM | 1597 | C | LEU | 1082 | 51.680 | −14.453 | 11.792 | 1.00 | 42.13 |
| ATOM | 1598 | O | LEU | 1082 | 51.514 | −15.050 | 10.724 | 1.00 | 42.31 |
| ATOM | 1599 | N | LEU | 1083 | 51.899 | −15.073 | 12.949 | 1.00 | 42.41 |
| ATOM | 1600 | CA | LEU | 1083 | 51.956 | −16.529 | 13.046 | 1.00 | 42.52 |
| ATOM | 1601 | CB | LEU | 1083 | 53.312 | −17.047 | 12.553 | 1.00 | 43.68 |
| ATOM | 1602 | CG | LEU | 1083 | 54.555 | −16.431 | 13.218 | 1.00 | 44.42 |
| ATOM | 1603 | CD1 | LEU | 1083 | 54.681 | −16.909 | 14.659 | 1.00 | 44.61 |
| ATOM | 1604 | CD2 | LEU | 1083 | 55.796 | −16.811 | 12.424 | 1.00 | 44.50 |
| ATOM | 1605 | C | LEU | 1083 | 51.773 | −16.914 | 14.494 | 1.00 | 42.49 |
| ATOM | 1606 | O | LEU | 1083 | 51.906 | −16.077 | 15.379 | 1.00 | 42.85 |
| ATOM | 1607 | N | ALA | 1084 | 51.476 | −18.182 | 14.741 | 1.00 | 42.91 |
| ATOM | 1608 | CA | ALA | 1084 | 51.293 | −18.652 | 16.108 | 1.00 | 43.06 |
| ATOM | 1609 | CB | ALA | 1084 | 49.976 | −19.396 | 16.238 | 1.00 | 42.67 |
| ATOM | 1610 | C | ALA | 1084 | 52.453 | −19.556 | 16.528 | 1.00 | 43.74 |
| ATOM | 1611 | O | ALA | 1084 | 52.552 | −20.712 | 16.101 | 1.00 | 44.02 |
| ATOM | 1612 | N | SER | 1085 | 53.325 | −19.006 | 17.365 | 1.00 | 43.87 |
| ATOM | 1613 | CA | SER | 1085 | 54.490 | −19.710 | 17.885 | 1.00 | 44.00 |
| ATOM | 1614 | CB | SER | 1085 | 55.496 | −18.679 | 18.409 | 1.00 | 44.79 |
| ATOM | 1615 | OG | SER | 1085 | 56.398 | −19.254 | 19.335 | 1.00 | 45.48 |
| ATOM | 1616 | C | SER | 1085 | 54.108 | −20.688 | 19.000 | 1.00 | 44.32 |
| ATOM | 1617 | O | SER | 1085 | 53.175 | −20.441 | 19.769 | 1.00 | 44.49 |
| ATOM | 1618 | N | LYS | 1086 | 54.835 | −21.798 | 19.079 | 1.00 | 44.70 |
| ATOM | 1619 | CA | LYS | 1086 | 54.588 | −22.820 | 20.094 | 1.00 | 45.19 |
| ATOM | 1620 | CB | LYS | 1086 | 55.266 | −24.131 | 19.675 | 1.00 | 46.52 |
| ATOM | 1621 | CG | LYS | 1086 | 54.703 | −25.397 | 20.312 | 1.00 | 47.78 |
| ATOM | 1622 | CD | LYS | 1086 | 53.331 | −25.743 | 19.748 | 1.00 | 49.13 |
| ATOM | 1623 | CE | LYS | 1086 | 52.910 | −27.155 | 20.151 | 1.00 | 49.88 |
| ATOM | 1624 | NZ | LYS | 1086 | 51.542 | −27.514 | 19.656 | 1.00 | 50.32 |
| ATOM | 1625 | C | LYS | 1086 | 55.139 | −22.366 | 21.452 | 1.00 | 44.74 |
| ATOM | 1626 | O | LYS | 1086 | 54.625 | −22.743 | 22.501 | 1.00 | 44.11 |
| ATOM | 1627 | N | SER | 1087 | 56.192 | −21.559 | 21.422 | 1.00 | 44.87 |
| ATOM | 1628 | CA | SER | 1087 | 56.804 | −21.063 | 22.650 | 1.00 | 45.28 |
| ATOM | 1629 | CB | SER | 1087 | 58.271 | −21.490 | 22.716 | 1.00 | 46.02 |
| ATOM | 1630 | OG | SER | 1087 | 58.975 | −21.066 | 21.558 | 1.00 | 47.68 |
| ATOM | 1631 | C | SER | 1087 | 56.710 | −19.548 | 22.713 | 1.00 | 45.02 |
| ATOM | 1632 | O | SER | 1087 | 56.562 | −18.886 | 21.691 | 1.00 | 45.40 |
| ATOM | 1633 | N | VAL | 1088 | 56.809 | −18.998 | 23.916 | 1.00 | 45.20 |
| ATOM | 1634 | CA | VAL | 1088 | 56.712 | −17.554 | 24.095 | 1.00 | 45.29 |
| ATOM | 1635 | CB | VAL | 1088 | 56.189 | −17.222 | 25.497 | 1.00 | 45.46 |
| ATOM | 1636 | CG1 | VAL | 1088 | 56.243 | −15.717 | 25.724 | 1.00 | 46.33 |
| ATOM | 1637 | CG2 | VAL | 1088 | 54.763 | −17.749 | 25.645 | 1.00 | 45.09 |
| ATOM | 1638 | C | VAL | 1088 | 58.016 | −16.806 | 23.866 | 1.00 | 44.97 |
| ATOM | 1639 | O | VAL | 1088 | 59.025 | −17.094 | 24.498 | 1.00 | 45.20 |
| ATOM | 1640 | N | THR | 1089 | 57.988 | −15.841 | 22.956 | 1.00 | 45.32 |
| ATOM | 1641 | CA | THR | 1089 | 59.170 | −15.035 | 22.658 | 1.00 | 45.53 |
| ATOM | 1642 | CB | THR | 1089 | 59.655 | −15.260 | 21.223 | 1.00 | 46.30 |
| ATOM | 1643 | OG1 | THR | 1089 | 58.949 | −14.383 | 20.333 | 1.00 | 47.07 |
| ATOM | 1644 | CG2 | THR | 1089 | 59.399 | −16.706 | 20.810 | 1.00 | 46.45 |
| ATOM | 1645 | C | THR | 1089 | 58.776 | −13.573 | 22.816 | 1.00 | 45.35 |
| ATOM | 1646 | O | THR | 1089 | 57.596 | −13.264 | 23.003 | 1.00 | 45.32 |
| ATOM | 1647 | N | ASP | 1090 | 59.747 | −12.672 | 22.739 | 1.00 | 45.33 |
| ATOM | 1648 | CA | ASP | 1090 | 59.434 | −11.261 | 22.902 | 1.00 | 45.38 |
| ATOM | 1649 | CB | ASP | 1090 | 60.713 | −10.431 | 23.010 | 1.00 | 45.99 |
| ATOM | 1650 | CG | ASP | 1090 | 61.720 | −10.772 | 21.941 | 1.00 | 47.95 |
| ATOM | 1651 | OD1 | ASP | 1090 | 61.310 | −11.144 | 20.816 | 1.00 | 47.57 |
| ATOM | 1652 | OD2 | ASP | 1090 | 62.933 | −10.655 | 22.226 | 1.00 | 49.45 |
| ATOM | 1653 | C | ASP | 1090 | 58.539 | −10.712 | 21.789 | 1.00 | 45.04 |
| ATOM | 1654 | O | ASP | 1090 | 58.078 | −9.572 | 21.867 | 1.00 | 45.86 |
| ATOM | 1655 | N | GLU | 1091 | 58.280 | −11.517 | 20.761 | 1.00 | 44.05 |
| ATOM | 1656 | CA | GLU | 1091 | 57.418 | −11.081 | 19.658 | 1.00 | 43.33 |
| ATOM | 1657 | CB | GLU | 1091 | 57.881 | −11.693 | 18.330 | 1.00 | 44.73 |
| ATOM | 1658 | CG | GLU | 1091 | 59.277 | −11.309 | 17.860 | 1.00 | 46.78 |
| ATOM | 1659 | CD | GLU | 1091 | 59.630 | −11.968 | 16.535 | 1.00 | 48.21 |
| ATOM | 1660 | OE1 | GLU | 1091 | 59.574 | −13.217 | 16.457 | 1.00 | 49.24 |
| ATOM | 1661 | OE2 | GLU | 1091 | 59.958 | −11.240 | 15.569 | 1.00 | 49.52 |
| ATOM | 1662 | C | GLU | 1091 | 55.961 | −11.508 | 19.905 | 1.00 | 42.20 |
| ATOM | 1663 | O | GLU | 1091 | 55.104 | −11.385 | 19.018 | 1.00 | 41.24 |
| ATOM | 1664 | N | CYS | 1092 | 55.692 | −12.015 | 21.106 | 1.00 | 40.91 |
| ATOM | 1665 | CA | CYS | 1092 | 54.359 | −12.493 | 21.468 | 1.00 | 39.88 |
| ATOM | 1666 | CB | CYS | 1092 | 54.460 | −13.848 | 22.164 | 1.00 | 40.43 |
| ATOM | 1667 | SG | CYS | 1092 | 54.899 | −15.188 | 21.085 | 1.00 | 42.83 |
| ATOM | 1668 | C | CYS | 1092 | 53.596 | −11.556 | 22.381 | 1.00 | 38.38 |
| ATOM | 1669 | O | CYS | 1092 | 52.503 | −11.894 | 22.851 | 1.00 | 37.68 |
| ATOM | 1670 | N | PHE | 1093 | 54.166 | −10.386 | 22.631 | 1.00 | 36.99 |
| ATOM | 1671 | CA | PHE | 1093 | 53.536 | −9.426 | 23.514 | 1.00 | 36.82 |
| ATOM | 1672 | CB | PHE | 1093 | 54.515 | −9.076 | 24.625 | 1.00 | 37.12 |
| ATOM | 1673 | CG | PHE | 1093 | 54.888 | −10.259 | 25.458 | 1.00 | 38.19 |
| ATOM | 1674 | CD1 | PHE | 1093 | 53.961 | −10.822 | 26.332 | 1.00 | 38.40 |
| ATOM | 1675 | CD2 | PHE | 1093 | 56.138 | −10.855 | 25.325 | 1.00 | 38.91 |
| ATOM | 1676 | CE1 | PHE | 1093 | 54.266 | −11.963 | 27.059 | 1.00 | 39.04 |
| ATOM | 1677 | CE2 | PHE | 1093 | 56.459 | −11.997 | 26.046 | 1.00 | 39.84 |
| ATOM | 1678 | CZ | PHE | 1093 | 55.520 | −12.557 | 26.919 | 1.00 | 39.76 |
| ATOM | 1679 | C | PHE | 1093 | 53.018 | −8.178 | 22.822 | 1.00 | 36.13 |
| ATOM | 1680 | O | PHE | 1093 | 53.686 | −7.595 | 21.968 | 1.00 | 35.71 |
| ATOM | 1681 | N | PHE | 1094 | 51.817 | −7.767 | 23.211 | 1.00 | 35.65 |
| ATOM | 1682 | CA | PHE | 1094 | 51.194 | −6.597 | 22.613 | 1.00 | 35.80 |
| ATOM | 1683 | CB | PHE | 1094 | 50.107 | −7.057 | 21.639 | 1.00 | 37.57 |
| ATOM | 1684 | CG | PHE | 1094 | 50.554 | −8.148 | 20.713 | 1.00 | 39.41 |
| ATOM | 1685 | CD1 | PHE | 1094 | 51.109 | −7.843 | 19.469 | 1.00 | 40.39 |
| ATOM | 1686 | CD2 | PHE | 1094 | 50.479 | −9.482 | 21.107 | 1.00 | 40.13 |
| ATOM | 1687 | CE1 | PHE | 1094 | 51.588 | −8.858 | 18.628 | 1.00 | 40.94 |
| ATOM | 1688 | CE2 | PHE | 1094 | 50.954 | −10.505 | 20.279 | 1.00 | 40.66 |
| ATOM | 1689 | CZ | PHE | 1094 | 51.511 | −10.191 | 19.037 | 1.00 | 40.89 |
| ATOM | 1690 | C | PHE | 1094 | 50.582 | −5.655 | 23.651 | 1.00 | 35.14 |
| ATOM | 1691 | O | PHE | 1094 | 50.145 | −6.087 | 24.720 | 1.00 | 35.22 |
| ATOM | 1692 | N | PHE | 1095 | 50.575 | −4.363 | 23.332 | 1.00 | 34.56 |
| ATOM | 1693 | CA | PHE | 1095 | 49.962 | −3.365 | 24.205 | 1.00 | 33.77 |
| ATOM | 1694 | CB | PHE | 1095 | 50.388 | −1.933 | 23.849 | 1.00 | 34.28 |
| ATOM | 1695 | CG | PHE | 1095 | 51.809 | −1.603 | 24.186 | 1.00 | 35.38 |
| ATOM | 1696 | CD1 | PHE | 1095 | 52.769 | −1.499 | 23.182 | 1.00 | 36.09 |
| ATOM | 1697 | CD2 | PHE | 1095 | 52.192 | −1.382 | 25.507 | 1.00 | 35.44 |
| ATOM | 1698 | CE1 | PHE | 1095 | 54.099 | −1.177 | 23.492 | 1.00 | 36.70 |
| ATOM | 1699 | CE2 | PHE | 1095 | 53.517 | −1.061 | 25.833 | 1.00 | 35.77 |
| ATOM | 1700 | CZ | PHE | 1095 | 54.473 | −0.957 | 24.827 | 1.00 | 36.05 |
| ATOM | 1701 | C | PHE | 1095 | 48.477 | −3.464 | 23.897 | 1.00 | 33.37 |
| ATOM | 1702 | O | PHE | 1095 | 48.071 | −3.230 | 22.757 | 1.00 | 34.29 |
| ATOM | 1703 | N | GLU | 1096 | 47.671 | −3.823 | 24.889 | 1.00 | 32.53 |
| ATOM | 1704 | CA | GLU | 1096 | 46.235 | −3.919 | 24.686 | 1.00 | 32.51 |
| ATOM | 1705 | CB | GLU | 1096 | 45.635 | −5.060 | 25.519 | 1.00 | 32.85 |
| ATOM | 1706 | CG | GLU | 1096 | 44.109 | −5.114 | 25.448 | 1.00 | 33.94 |
| ATOM | 1707 | CD | GLU | 1096 | 43.518 | −6.305 | 26.174 | 1.00 | 34.39 |
| ATOM | 1708 | OE1 | GLU | 1096 | 43.805 | −6.463 | 27.380 | 1.00 | 34.98 |
| ATOM | 1709 | OE2 | GLU | 1096 | 42.761 | −7.081 | 25.543 | 1.00 | 34.04 |
| ATOM | 1710 | C | GLU | 1096 | 45.624 | −2.599 | 25.120 | 1.00 | 32.26 |
| ATOM | 1711 | O | GLU | 1096 | 45.751 | −2.205 | 26.275 | 1.00 | 32.54 |
| ATOM | 1712 | N | ARG | 1097 | 44.975 | −1.900 | 24.201 | 1.00 | 32.08 |
| ATOM | 1713 | CA | ARG | 1097 | 44.372 | −0.642 | 24.580 | 1.00 | 31.95 |
| ATOM | 1714 | CB | ARG | 1097 | 45.135 | 0.537 | 23.971 | 1.00 | 33.89 |
| ATOM | 1715 | CG | ARG | 1097 | 44.688 | 1.882 | 24.547 | 1.00 | 38.54 |
| ATOM | 1716 | CD | ARG | 1097 | 45.368 | 3.092 | 23.885 | 1.00 | 40.80 |
| ATOM | 1717 | NE | ARG | 1097 | 44.670 | 4.347 | 24.186 | 1.00 | 42.26 |
| ATOM | 1718 | CZ | ARG | 1097 | 44.554 | 4.881 | 25.403 | 1.00 | 43.84 |
| ATOM | 1719 | NH1 | ARG | 1097 | 45.093 | 4.281 | 26.464 | 1.00 | 44.26 |
| ATOM | 1720 | NH2 | ARG | 1097 | 43.886 | 6.018 | 25.561 | 1.00 | 43.98 |
| ATOM | 1721 | C | ARG | 1097 | 42.904 | −0.565 | 24.211 | 1.00 | 30.60 |
| ATOM | 1722 | O | ARG | 1097 | 42.503 | −0.946 | 23.107 | 1.00 | 31.12 |
| ATOM | 1723 | N | LEU | 1098 | 42.101 | −0.111 | 25.171 | 1.00 | 29.15 |
| ATOM | 1724 | CA | LEU | 1098 | 40.666 | 0.075 | 24.968 | 1.00 | 27.56 |
| ATOM | 1725 | CB | LEU | 1098 | 39.904 | −0.110 | 26.285 | 1.00 | 26.27 |
| ATOM | 1726 | CG | LEU | 1098 | 38.460 | 0.400 | 26.340 | 1.00 | 25.48 |
| ATOM | 1727 | CD1 | LEU | 1098 | 37.731 | 0.042 | 25.065 | 1.00 | 25.37 |
| ATOM | 1728 | CD2 | LEU | 1098 | 37.737 | −0.200 | 27.538 | 1.00 | 24.82 |
| ATOM | 1729 | C | LEU | 1098 | 40.574 | 1.513 | 24.477 | 1.00 | 27.43 |
| ATOM | 1730 | O | LEU | 1098 | 40.626 | 2.461 | 25.266 | 1.00 | 27.06 |
| ATOM | 1731 | N | GLU | 1099 | 40.470 | 1.664 | 23.163 | 1.00 | 27.08 |
| ATOM | 1732 | CA | GLU | 1099 | 40.429 | 2.978 | 22.566 | 1.00 | 27.36 |
| ATOM | 1733 | CB | GLU | 1099 | 40.682 | 2.842 | 21.068 | 1.00 | 28.55 |
| ATOM | 1734 | CG | GLU | 1099 | 41.949 | 2.016 | 20.751 | 1.00 | 30.06 |
| ATOM | 1735 | CD | GLU | 1099 | 43.276 | 2.783 | 20.950 | 1.00 | 31.97 |
| ATOM | 1736 | OE1 | GLU | 1099 | 44.356 | 2.186 | 20.699 | 1.00 | 32.77 |
| ATOM | 1737 | OE2 | GLU | 1099 | 43.251 | 3.973 | 21.342 | 1.00 | 32.35 |
| ATOM | 1738 | C | GLU | 1099 | 39.139 | 3.732 | 22.871 | 1.00 | 27.47 |
| ATOM | 1739 | O | GLU | 1099 | 38.135 | 3.154 | 23.300 | 1.00 | 27.15 |
| ATOM | 1740 | N | SER | 1100 | 39.187 | 5.042 | 22.674 | 1.00 | 27.31 |
| ATOM | 1741 | CA | SER | 1100 | 38.046 | 5.906 | 22.933 | 1.00 | 26.63 |
| ATOM | 1742 | CB | SER | 1100 | 38.446 | 7.353 | 22.682 | 1.00 | 25.98 |
| ATOM | 1743 | OG | SER | 1100 | 38.955 | 7.509 | 21.366 | 1.00 | 27.10 |
| ATOM | 1744 | C | SER | 1100 | 36.811 | 5.550 | 22.095 | 1.00 | 26.75 |
| ATOM | 1745 | O | SER | 1100 | 35.697 | 5.961 | 22.408 | 1.00 | 26.58 |
| ATOM | 1746 | N | ASN | 1101 | 37.009 | 4.787 | 21.031 | 1.00 | 26.49 |
| ATOM | 1747 | CA | ASN | 1101 | 35.892 | 4.400 | 20.195 | 1.00 | 27.31 |
| ATOM | 1748 | CB | ASN | 1101 | 36.344 | 4.274 | 18.737 | 1.00 | 28.53 |
| ATOM | 1749 | CG | ASN | 1101 | 37.499 | 3.301 | 18.554 | 1.00 | 29.91 |
| ATOM | 1750 | OD1 | ASN | 1101 | 37.735 | 2.425 | 19.393 | 1.00 | 31.60 |
| ATOM | 1751 | ND2 | ASN | 1101 | 38.214 | 3.439 | 17.439 | 1.00 | 29.25 |
| ATOM | 1752 | C | ASN | 1101 | 35.281 | 3.089 | 20.671 | 1.00 | 27.04 |
| ATOM | 1753 | O | ASN | 1101 | 34.416 | 2.530 | 20.001 | 1.00 | 27.41 |
| ATOM | 1754 | N | ASN | 1102 | 35.750 | 2.610 | 21.825 | 1.00 | 26.96 |
| ATOM | 1755 | CA | ASN | 1102 | 35.277 | 1.377 | 22.452 | 1.00 | 26.80 |
| ATOM | 1756 | CB | ASN | 1102 | 33.761 | 1.399 | 22.553 | 1.00 | 28.13 |
| ATOM | 1757 | CG | ASN | 1102 | 33.272 | 2.488 | 23.463 | 1.00 | 30.06 |
| ATOM | 1758 | OD1 | ASN | 1102 | 33.577 | 2.493 | 24.664 | 1.00 | 31.18 |
| ATOM | 1759 | ND2 | ASN | 1102 | 32.515 | 3.431 | 22.906 | 1.00 | 30.51 |
| ATOM | 1760 | C | ASN | 1102 | 35.724 | 0.071 | 21.821 | 1.00 | 27.01 |
| ATOM | 1761 | O | ASN | 1102 | 35.153 | −0.983 | 22.089 | 1.00 | 27.75 |
| ATOM | 1762 | N | TYR | 1103 | 36.737 | 0.139 | 20.973 | 1.00 | 26.85 |
| ATOM | 1763 | CA | TYR | 1103 | 37.272 | −1.059 | 20.349 | 1.00 | 27.78 |
| ATOM | 1764 | CB | TYR | 1103 | 37.368 | −0.872 | 18.843 | 1.00 | 29.04 |
| ATOM | 1765 | CG | TYR | 1103 | 36.085 | −1.122 | 18.094 | 1.00 | 30.29 |
| ATOM | 1766 | CD1 | TYR | 1103 | 35.777 | −2.399 | 17.615 | 1.00 | 30.52 |
| ATOM | 1767 | CE1 | TYR | 1103 | 34.622 | −2.628 | 16.871 | 1.00 | 29.96 |
| ATOM | 1768 | CD2 | TYR | 1103 | 35.196 | −0.076 | 17.815 | 1.00 | 30.41 |
| ATOM | 1769 | CE2 | TYR | 1103 | 34.032 | −0.300 | 17.071 | 1.00 | 30.20 |
| ATOM | 1770 | CZ | TYR | 1103 | 33.756 | −1.576 | 16.606 | 1.00 | 30.55 |
| ATOM | 1771 | OH | TYR | 1103 | 32.608 | −1.813 | 15.886 | 1.00 | 31.37 |
| ATOM | 1772 | C | TYR | 1103 | 38.668 | −1.210 | 20.937 | 1.00 | 27.43 |
| ATOM | 1773 | O | TYR | 1103 | 39.238 | −0.240 | 21.436 | 1.00 | 26.33 |
| ATOM | 1774 | N | ASN | 1104 | 39.200 | −2.426 | 20.895 | 1.00 | 27.31 |
| ATOM | 1775 | CA | ASN | 1104 | 40.535 | −2.705 | 21.412 | 1.00 | 27.80 |
| ATOM | 1776 | CB | ASN | 1104 | 40.568 | −4.085 | 22.068 | 1.00 | 28.04 |
| ATOM | 1777 | CG | ASN | 1104 | 39.922 | −4.102 | 23.421 | 1.00 | 28.68 |
| ATOM | 1778 | OD1 | ASN | 1104 | 39.020 | −3.310 | 23.704 | 1.00 | 29.89 |
| ATOM | 1779 | ND2 | ASN | 1104 | 40.362 | −5.018 | 24.267 | 1.00 | 28.26 |
| ATOM | 1780 | C | ASN | 1104 | 41.531 | −2.723 | 20.264 | 1.00 | 27.87 |
| ATOM | 1781 | O | ASN | 1104 | 41.173 | −3.060 | 19.137 | 1.00 | 27.95 |
| ATOM | 1782 | N | THR | 1105 | 42.774 | −2.354 | 20.550 | 1.00 | 27.75 |
| ATOM | 1783 | CA | THR | 1105 | 43.827 | −2.409 | 19.554 | 1.00 | 28.28 |
| ATOM | 1784 | CB | THR | 1105 | 44.405 | −1.022 | 19.218 | 1.00 | 28.48 |
| ATOM | 1785 | OG1 | THR | 1105 | 44.794 | −0.359 | 20.420 | 1.00 | 28.92 |
| ATOM | 1786 | CG2 | THR | 1105 | 43.385 | −0.181 | 18.482 | 1.00 | 28.84 |
| ATOM | 1787 | C | THR | 1105 | 44.916 | −3.239 | 20.198 | 1.00 | 28.97 |
| ATOM | 1788 | O | THR | 1105 | 44.985 | −3.330 | 21.424 | 1.00 | 29.37 |
| ATOM | 1789 | N | TYR | 1106 | 45.758 | −3.855 | 19.376 | 1.00 | 30.18 |
| ATOM | 1790 | CA | TYR | 1106 | 46.863 | −4.677 | 19.870 | 1.00 | 31.08 |
| ATOM | 1791 | CB | TYR | 1106 | 46.554 | −6.157 | 19.646 | 1.00 | 30.84 |
| ATOM | 1792 | CG | TYR | 1106 | 45.439 | −6.630 | 20.538 | 1.00 | 31.35 |
| ATOM | 1793 | CD1 | TYR | 1106 | 45.688 | −7.002 | 21.859 | 1.00 | 31.34 |
| ATOM | 1794 | CE1 | TYR | 1106 | 44.649 | −7.301 | 22.732 | 1.00 | 32.19 |
| ATOM | 1795 | CD2 | TYR | 1106 | 44.116 | −6.583 | 20.108 | 1.00 | 32.07 |
| ATOM | 1796 | CE2 | TYR | 1106 | 43.063 | −6.879 | 20.979 | 1.00 | 32.08 |
| ATOM | 1797 | CZ | TYR | 1106 | 43.339 | −7.228 | 22.287 | 1.00 | 31.83 |
| ATOM | 1798 | OH | TYR | 1106 | 42.304 | −7.441 | 23.160 | 1.00 | 32.96 |
| ATOM | 1799 | C | TYR | 1106 | 48.125 | −4.275 | 19.130 | 1.00 | 31.71 |
| ATOM | 1800 | O | TYR | 1106 | 48.318 | −4.625 | 17.974 | 1.00 | 31.24 |
| ATOM | 1801 | N | ARG | 1107 | 48.977 | −3.529 | 19.818 | 1.00 | 33.58 |
| ATOM | 1802 | CA | ARG | 1107 | 50.224 | −3.018 | 19.253 | 1.00 | 35.32 |
| ATOM | 1803 | CB | ARG | 1107 | 50.424 | −1.581 | 19.746 | 1.00 | 36.45 |
| ATOM | 1804 | CG | ARG | 1107 | 51.164 | −0.651 | 18.802 | 1.00 | 39.83 |
| ATOM | 1805 | CD | ARG | 1107 | 51.259 | 0.757 | 19.402 | 1.00 | 42.36 |
| ATOM | 1806 | NE | ARG | 1107 | 52.243 | 0.836 | 20.482 | 1.00 | 44.58 |
| ATOM | 1807 | CZ | ARG | 1107 | 52.394 | 1.884 | 21.287 | 1.00 | 45.48 |
| ATOM | 1808 | NH1 | ARG | 1107 | 51.617 | 2.947 | 21.145 | 1.00 | 46.20 |
| ATOM | 1809 | NH2 | ARG | 1107 | 53.340 | 1.885 | 22.222 | 1.00 | 46.88 |
| ATOM | 1810 | C | ARG | 1107 | 51.428 | −3.876 | 19.670 | 1.00 | 35.84 |
| ATOM | 1811 | O | ARG | 1107 | 51.599 | −4.197 | 20.851 | 1.00 | 35.30 |
| ATOM | 1812 | N | SER | 1108 | 52.258 | −4.239 | 18.697 | 1.00 | 36.59 |
| ATOM | 1813 | CA | SER | 1108 | 53.451 | −5.048 | 18.954 | 1.00 | 38.06 |
| ATOM | 1814 | CB | SER | 1108 | 54.222 | −5.272 | 17.647 | 1.00 | 38.77 |
| ATOM | 1815 | OG | SER | 1108 | 55.434 | −5.965 | 17.883 | 1.00 | 40.41 |
| ATOM | 1816 | C | SER | 1108 | 54.355 | −4.326 | 19.938 | 1.00 | 38.32 |
| ATOM | 1817 | O | SER | 1108 | 54.683 | −3.161 | 19.726 | 1.00 | 38.65 |
| ATOM | 1818 | N | ARG | 1109 | 54.764 | −4.995 | 21.013 | 1.00 | 39.66 |
| ATOM | 1819 | CA | ARG | 1109 | 55.642 | −4.328 | 21.971 | 1.00 | 41.41 |
| ATOM | 1820 | CB | ARG | 1109 | 55.616 | −5.020 | 23.352 | 1.00 | 42.33 |
| ATOM | 1821 | CG | ARG | 1109 | 56.498 | −4.298 | 24.395 | 1.00 | 44.15 |
| ATOM | 1822 | CD | ARG | 1109 | 56.350 | −4.810 | 25.835 | 1.00 | 45.05 |
| ATOM | 1823 | NE | ARG | 1109 | 56.616 | −6.244 | 25.986 | 1.00 | 46.35 |
| ATOM | 1824 | CZ | ARG | 1109 | 56.880 | −6.848 | 27.146 | 1.00 | 47.17 |
| ATOM | 1825 | NH1 | ARG | 1109 | 56.928 | −6.152 | 28.277 | 1.00 | 48.51 |
| ATOM | 1826 | NH2 | ARG | 1109 | 57.081 | −8.158 | 27.184 | 1.00 | 47.56 |
| ATOM | 1827 | C | ARG | 1109 | 57.064 | −4.297 | 21.403 | 1.00 | 42.12 |
| ATOM | 1828 | O | ARG | 1109 | 57.894 | −3.472 | 21.804 | 1.00 | 42.27 |
| ATOM | 1829 | N | LYS | 1110 | 57.319 | −5.192 | 20.449 | 1.00 | 42.61 |
| ATOM | 1830 | CA | LYS | 1110 | 58.616 | −5.303 | 19.784 | 1.00 | 43.20 |
| ATOM | 1831 | CB | LYS | 1110 | 58.756 | −6.717 | 19.199 | 1.00 | 44.38 |
| ATOM | 1832 | CG | LYS | 1110 | 60.179 | −7.161 | 18.895 | 1.00 | 45.87 |
| ATOM | 1833 | CD | LYS | 1110 | 60.961 | −7.368 | 20.183 | 1.00 | 46.71 |
| ATOM | 1834 | CE | LYS | 1110 | 62.347 | −7.960 | 19.934 | 1.00 | 46.54 |
| ATOM | 1835 | NZ | LYS | 1110 | 63.039 | −8.205 | 21.240 | 1.00 | 46.44 |
| ATOM | 1836 | C | LYS | 1110 | 58.681 | −4.268 | 18.653 | 1.00 | 42.57 |
| ATOM | 1837 | O | LYS | 1110 | 59.601 | −3.439 | 18.580 | 1.00 | 42.84 |
| ATOM | 1838 | N | TYR | 1111 | 57.686 | −4.335 | 17.773 | 1.00 | 41.61 |
| ATOM | 1839 | CA | TYR | 1111 | 57.579 | −3.429 | 16.630 | 1.00 | 41.13 |
| ATOM | 1840 | CB | TYR | 1111 | 57.245 | −4.258 | 15.396 | 1.00 | 41.65 |
| ATOM | 1841 | CG | TYR | 1111 | 58.142 | −5.475 | 15.302 | 1.00 | 42.52 |
| ATOM | 1842 | CD1 | TYR | 1111 | 59.525 | −5.332 | 15.194 | 1.00 | 42.63 |
| ATOM | 1843 | CE1 | TYR | 1111 | 60.361 | −6.436 | 15.140 | 1.00 | 43.26 |
| ATOM | 1844 | CD2 | TYR | 1111 | 57.615 | −6.766 | 15.356 | 1.00 | 42.98 |
| ATOM | 1845 | CE2 | TYR | 1111 | 58.443 | −7.882 | 15.302 | 1.00 | 43.72 |
| ATOM | 1846 | CZ | TYR | 1111 | 59.819 | −7.710 | 15.190 | 1.00 | 43.91 |
| ATOM | 1847 | OH | TYR | 1111 | 60.650 | −8.806 | 15.091 | 1.00 | 43.80 |
| ATOM | 1848 | C | TYR | 1111 | 56.483 | −2.425 | 16.961 | 1.00 | 40.38 |
| ATOM | 1849 | O | TYR | 1111 | 55.397 | −2.444 | 16.401 | 1.00 | 39.87 |
| ATOM | 1850 | N | THR | 1112 | 56.817 | −1.551 | 17.902 | 1.00 | 40.63 |
| ATOM | 1851 | CA | THR | 1112 | 55.938 | −0.526 | 18.452 | 1.00 | 40.34 |
| ATOM | 1852 | CB | THR | 1112 | 56.766 | 0.475 | 19.238 | 1.00 | 40.33 |
| ATOM | 1853 | OG1 | THR | 1112 | 57.629 | 1.179 | 18.335 | 1.00 | 40.43 |
| ATOM | 1854 | CG2 | THR | 1112 | 57.597 | −0.248 | 20.295 | 1.00 | 39.52 |
| ATOM | 1855 | C | THR | 1112 | 54.980 | 0.272 | 17.572 | 1.00 | 40.34 |
| ATOM | 1856 | O | THR | 1112 | 54.088 | 0.930 | 18.103 | 1.00 | 40.94 |
| ATOM | 1857 | N | SER | 1113 | 55.137 | 0.234 | 16.254 | 1.00 | 39.79 |
| ATOM | 1858 | CA | SER | 1113 | 54.239 | 1.004 | 15.396 | 1.00 | 39.57 |
| ATOM | 1859 | CB | SER | 1113 | 55.038 | 1.910 | 14.451 | 1.00 | 40.55 |
| ATOM | 1860 | OG | SER | 1113 | 55.922 | 2.757 | 15.172 | 1.00 | 43.39 |
| ATOM | 1861 | C | SER | 1113 | 53.321 | 0.125 | 14.564 | 1.00 | 39.13 |
| ATOM | 1862 | O | SER | 1113 | 52.564 | 0.623 | 13.728 | 1.00 | 39.03 |
| ATOM | 1863 | N | TRP | 1114 | 53.380 | −1.182 | 14.781 | 1.00 | 38.56 |
| ATOM | 1864 | CA | TRP | 1114 | 52.532 | −2.077 | 14.008 | 1.00 | 38.50 |
| ATOM | 1865 | CB | TRP | 1114 | 53.368 | −3.157 | 13.316 | 1.00 | 41.45 |
| ATOM | 1866 | CG | TRP | 1114 | 54.439 | −2.620 | 12.398 | 1.00 | 44.70 |
| ATOM | 1867 | CD2 | TRP | 1114 | 55.593 | −3.326 | 11.931 | 1.00 | 45.63 |
| ATOM | 1868 | CE2 | TRP | 1114 | 56.298 | −2.451 | 11.068 | 1.00 | 46.45 |
| ATOM | 1869 | CE3 | TRP | 1114 | 56.100 | −4.613 | 12.157 | 1.00 | 46.01 |
| ATOM | 1870 | CD1 | TRP | 1114 | 54.488 | −1.378 | 11.814 | 1.00 | 45.33 |
| ATOM | 1871 | NE1 | TRP | 1114 | 55.601 | −1.273 | 11.016 | 1.00 | 45.59 |
| ATOM | 1872 | CZ2 | TRP | 1114 | 57.488 | −2.827 | 10.426 | 1.00 | 46.87 |
| ATOM | 1873 | CZ3 | TRP | 1114 | 57.284 | −4.990 | 11.523 | 1.00 | 46.92 |
| ATOM | 1874 | CH2 | TRP | 1114 | 57.966 | −4.095 | 10.665 | 1.00 | 47.25 |
| ATOM | 1875 | C | TRP | 1114 | 51.449 | −2.729 | 14.854 | 1.00 | 37.03 |
| ATOM | 1876 | O | TRP | 1114 | 51.692 | −3.114 | 16.003 | 1.00 | 36.42 |
| ATOM | 1877 | N | TYR | 1115 | 50.257 | −2.851 | 14.271 | 1.00 | 35.04 |
| ATOM | 1878 | CA | TYR | 1115 | 49.123 | −3.444 | 14.963 | 1.00 | 33.88 |
| ATOM | 1879 | CB | TYR | 1115 | 47.893 | −2.536 | 14.906 | 1.00 | 34.58 |
| ATOM | 1880 | CG | TYR | 1115 | 48.052 | −1.172 | 15.530 | 1.00 | 35.50 |
| ATOM | 1881 | CD1 | TYR | 1115 | 48.688 | −0.138 | 14.843 | 1.00 | 35.22 |
| ATOM | 1882 | CE1 | TYR | 1115 | 48.803 | 1.121 | 15.403 | 1.00 | 35.65 |
| ATOM | 1883 | CD2 | TYR | 1115 | 47.539 | −0.906 | 16.804 | 1.00 | 35.62 |
| ATOM | 1884 | CE2 | TYR | 1115 | 47.651 | 0.353 | 17.374 | 1.00 | 35.20 |
| ATOM | 1885 | CZ | TYR | 1115 | 48.281 | 1.360 | 16.669 | 1.00 | 35.90 |
| ATOM | 1886 | OH | TYR | 1115 | 48.394 | 2.612 | 17.226 | 1.00 | 37.37 |
| ATOM | 1887 | C | TYR | 1115 | 48.696 | −4.772 | 14.391 | 1.00 | 33.69 |
| ATOM | 1888 | O | TYR | 1115 | 48.922 | −5.068 | 13.218 | 1.00 | 34.06 |
| ATOM | 1889 | N | VAL | 1116 | 48.062 | −5.575 | 15.234 | 1.00 | 33.42 |
| ATOM | 1890 | CA | VAL | 1116 | 47.532 | −6.849 | 14.793 | 1.00 | 33.12 |
| ATOM | 1891 | CB | VAL | 1116 | 47.054 | −7.695 | 15.990 | 1.00 | 33.51 |
| ATOM | 1892 | CG1 | VAL | 1116 | 46.150 | −8.821 | 15.511 | 1.00 | 33.27 |
| ATOM | 1893 | CG2 | VAL | 1116 | 48.261 | −8.261 | 16.731 | 1.00 | 33.38 |
| ATOM | 1894 | C | VAL | 1116 | 46.341 | −6.409 | 13.960 | 1.00 | 33.39 |
| ATOM | 1895 | O | VAL | 1116 | 45.609 | −5.513 | 14.367 | 1.00 | 33.92 |
| ATOM | 1896 | N | ALA | 1117 | 46.145 | −7.011 | 12.792 | 1.00 | 34.03 |
| ATOM | 1897 | CA | ALA | 1117 | 45.029 | −6.610 | 11.941 | 1.00 | 33.77 |
| ATOM | 1898 | CB | ALA | 1117 | 45.385 | −5.340 | 11.204 | 1.00 | 32.85 |
| ATOM | 1899 | C | ALA | 1117 | 44.589 | −7.666 | 10.941 | 1.00 | 34.98 |
| ATOM | 1900 | O | ALA | 1117 | 45.370 | −8.534 | 10.557 | 1.00 | 35.51 |
| ATOM | 1901 | N | LEU | 1118 | 43.327 | −7.583 | 10.525 | 1.00 | 36.34 |
| ATOM | 1902 | CA | LEU | 1118 | 42.766 | −8.502 | 9.539 | 1.00 | 37.72 |
| ATOM | 1903 | CB | LEU | 1118 | 41.626 | −9.326 | 10.129 | 1.00 | 37.37 |
| ATOM | 1904 | CG | LEU | 1118 | 41.895 | −10.231 | 11.327 | 1.00 | 37.62 |
| ATOM | 1905 | CD1 | LEU | 1118 | 40.633 | −11.057 | 11.572 | 1.00 | 37.99 |
| ATOM | 1906 | CD2 | LEU | 1118 | 43.094 | −11.142 | 11.073 | 1.00 | 37.31 |
| ATOM | 1907 | C | LEU | 1118 | 42.218 | −7.693 | 8.373 | 1.00 | 39.42 |
| ATOM | 1908 | O | LEU | 1118 | 41.644 | −6.621 | 8.580 | 1.00 | 39.97 |
| ATOM | 1909 | N | LYS | 1119 | 42.397 | −8.202 | 7.152 | 1.00 | 41.10 |
| ATOM | 1910 | CA | LYS | 1119 | 41.897 | −7.530 | 5.955 | 1.00 | 42.60 |
| ATOM | 1911 | CB | LYS | 1119 | 42.641 | −7.988 | 4.707 | 1.00 | 43.98 |
| ATOM | 1912 | CG | LYS | 1119 | 44.155 | −7.954 | 4.768 | 1.00 | 46.36 |
| ATOM | 1913 | CD | LYS | 1119 | 44.728 | −8.548 | 3.474 | 1.00 | 48.39 |
| ATOM | 1914 | CE | LYS | 1119 | 44.032 | −9.891 | 3.145 | 1.00 | 50.47 |
| ATOM | 1915 | NZ | LYS | 1119 | 44.456 | −10.585 | 1.870 | 1.00 | 51.28 |
| ATOM | 1916 | C | LYS | 1119 | 40.435 | −7.911 | 5.788 | 1.00 | 43.59 |
| ATOM | 1917 | O | LYS | 1119 | 39.939 | −8.816 | 6.461 | 1.00 | 43.21 |
| ATOM | 1918 | N | ARG | 1120 | 39.747 | −7.240 | 4.872 | 1.00 | 44.93 |
| ATOM | 1919 | CA | ARG | 1120 | 38.341 | −7.542 | 4.662 | 1.00 | 46.92 |
| ATOM | 1920 | CB | ARG | 1120 | 37.691 | −6.438 | 3.821 | 1.00 | 48.95 |
| ATOM | 1921 | CG | ARG | 1120 | 36.178 | −6.395 | 3.938 | 1.00 | 51.51 |
| ATOM | 1922 | CD | ARG | 1120 | 35.664 | −4.995 | 3.676 | 1.00 | 54.28 |
| ATOM | 1923 | NE | ARG | 1120 | 34.208 | −4.946 | 3.522 | 1.00 | 57.28 |
| ATOM | 1924 | CZ | ARG | 1120 | 33.534 | −5.519 | 2.522 | 1.00 | 58.66 |
| ATOM | 1925 | NH1 | ARG | 1120 | 34.174 | −6.201 | 1.578 | 1.00 | 59.34 |
| ATOM | 1926 | NH2 | ARG | 1120 | 32.215 | −5.391 | 2.448 | 1.00 | 59.25 |
| ATOM | 1927 | C | ARG | 1120 | 38.157 | −8.924 | 4.021 | 1.00 | 47.06 |
| ATOM | 1928 | O | ARG | 1120 | 37.036 | −9.432 | 3.923 | 1.00 | 47.19 |
| ATOM | 1929 | N | THR | 1121 | 39.267 | −9.539 | 3.617 | 1.00 | 46.62 |
| ATOM | 1930 | CA | THR | 1121 | 39.243 | −10.866 | 3.000 | 1.00 | 46.46 |
| ATOM | 1931 | CB | THR | 1121 | 40.454 | −11.063 | 2.080 | 1.00 | 47.24 |
| ATOM | 1932 | OG1 | THR | 1121 | 41.620 | −11.309 | 2.880 | 1.00 | 46.73 |
| ATOM | 1933 | CG2 | THR | 1121 | 40.687 | −9.817 | 1.223 | 1.00 | 47.30 |
| ATOM | 1934 | C | THR | 1121 | 39.298 | −11.974 | 4.052 | 1.00 | 46.56 |
| ATOM | 1935 | O | THR | 1121 | 39.097 | −13.151 | 3.738 | 1.00 | 46.77 |
| ATOM | 1936 | N | GLY | 1122 | 39.584 | −11.598 | 5.296 | 1.00 | 46.47 |
| ATOM | 1937 | CA | GLY | 1122 | 39.688 | −12.583 | 6.356 | 1.00 | 46.18 |
| ATOM | 1938 | C | GLY | 1122 | 41.136 | −13.008 | 6.555 | 1.00 | 46.40 |
| ATOM | 1939 | O | GLY | 1122 | 41.441 | −13.817 | 7.431 | 1.00 | 46.65 |
| ATOM | 1940 | N | GLN | 1123 | 42.031 | −12.476 | 5.728 | 1.00 | 46.45 |
| ATOM | 1941 | CA | GLN | 1123 | 43.446 | −12.800 | 5.843 | 1.00 | 46.44 |
| ATOM | 1942 | CB | GLN | 1123 | 44.118 | −12.827 | 4.464 | 1.00 | 47.74 |
| ATOM | 1943 | CG | GLN | 1123 | 43.488 | −13.815 | 3.472 | 1.00 | 49.49 |
| ATOM | 1944 | CD | GLN | 1123 | 43.315 | −15.217 | 4.052 | 1.00 | 50.46 |
| ATOM | 1945 | OE1 | GLN | 1123 | 44.280 | −15.835 | 4.511 | 1.00 | 50.51 |
| ATOM | 1946 | NE2 | GLN | 1123 | 42.078 | −15.725 | 4.031 | 1.00 | 51.06 |
| ATOM | 1947 | C | GLN | 1123 | 44.045 | −11.712 | 6.711 | 1.00 | 46.16 |
| ATOM | 1948 | O | GLN | 1123 | 43.580 | −10.572 | 6.686 | 1.00 | 46.45 |
| ATOM | 1949 | N | TYR | 1124 | 45.060 | −12.056 | 7.494 | 1.00 | 45.59 |
| ATOM | 1950 | CA | TYR | 1124 | 45.663 | −11.065 | 8.368 | 1.00 | 45.15 |
| ATOM | 1951 | CB | TYR | 1124 | 46.702 | −11.713 | 9.286 | 1.00 | 46.41 |
| ATOM | 1952 | CG | TYR | 1124 | 48.008 | −12.092 | 8.623 | 1.00 | 47.85 |
| ATOM | 1953 | CD1 | TYR | 1124 | 49.017 | −11.149 | 8.430 | 1.00 | 48.14 |
| ATOM | 1954 | CE1 | TYR | 1124 | 50.247 | −11.516 | 7.882 | 1.00 | 49.56 |
| ATOM | 1955 | CD2 | TYR | 1124 | 48.256 | −13.412 | 8.240 | 1.00 | 48.31 |
| ATOM | 1956 | CE2 | TYR | 1124 | 49.478 | −13.787 | 7.692 | 1.00 | 49.06 |
| ATOM | 1957 | CZ | TYR | 1124 | 50.469 | −12.839 | 7.518 | 1.00 | 49.67 |
| ATOM | 1958 | OH | TYR | 1124 | 51.686 | −13.222 | 6.999 | 1.00 | 50.27 |
| ATOM | 1959 | C | TYR | 1124 | 46.303 | −10.000 | 7.507 | 1.00 | 44.57 |
| ATOM | 1960 | O | TYR | 1124 | 46.589 | −10.238 | 6.339 | 1.00 | 44.10 |
| ATOM | 1961 | N | LYS | 1125 | 46.521 | −8.828 | 8.089 | 1.00 | 44.03 |
| ATOM | 1962 | CA | LYS | 1125 | 47.121 | −7.709 | 7.379 | 1.00 | 44.18 |
| ATOM | 1963 | CB | LYS | 1125 | 46.228 | −6.479 | 7.559 | 1.00 | 44.86 |
| ATOM | 1964 | CG | LYS | 1125 | 46.718 | −5.207 | 6.917 | 1.00 | 45.01 |
| ATOM | 1965 | CD | LYS | 1125 | 45.663 | −4.127 | 7.069 | 1.00 | 45.88 |
| ATOM | 1966 | CE | LYS | 1125 | 46.028 | −2.868 | 6.303 | 1.00 | 46.72 |
| ATOM | 1967 | NZ | LYS | 1125 | 44.874 | −1.924 | 6.238 | 1.00 | 47.66 |
| ATOM | 1968 | C | LYS | 1125 | 48.505 | −7.444 | 7.955 | 1.00 | 44.32 |
| ATOM | 1969 | O | LYS | 1125 | 48.649 | −7.340 | 9.171 | 1.00 | 44.58 |
| ATOM | 1970 | N | LEU | 1126 | 49.520 | −7.345 | 7.093 | 1.00 | 44.53 |
| ATOM | 1971 | CA | LEU | 1126 | 50.887 | −7.083 | 7.553 | 1.00 | 44.49 |
| ATOM | 1972 | CB | LEU | 1126 | 51.827 | −6.787 | 6.377 | 1.00 | 45.65 |
| ATOM | 1973 | CG | LEU | 1126 | 52.291 | −7.883 | 5.414 | 1.00 | 46.87 |
| ATOM | 1974 | CD1 | LEU | 1126 | 53.446 | −7.318 | 4.596 | 1.00 | 46.95 |
| ATOM | 1975 | CD2 | LEU | 1126 | 52.756 | −9.133 | 6.174 | 1.00 | 47.73 |
| ATOM | 1976 | C | LEU | 1126 | 50.936 | −5.898 | 8.515 | 1.00 | 44.52 |
| ATOM | 1977 | O | LEU | 1126 | 50.411 | −4.818 | 8.223 | 1.00 | 44.23 |
| ATOM | 1978 | N | GLY | 1127 | 51.577 | −6.102 | 9.661 | 1.00 | 44.10 |
| ATOM | 1979 | CA | GLY | 1127 | 51.673 | −5.037 | 10.635 | 1.00 | 43.79 |
| ATOM | 1980 | C | GLY | 1127 | 52.362 | −3.830 | 10.038 | 1.00 | 43.95 |
| ATOM | 1981 | O | GLY | 1127 | 52.069 | −2.688 | 10.398 | 1.00 | 43.61 |
| ATOM | 1982 | N | SER | 1128 | 53.280 | −4.082 | 9.113 | 1.00 | 44.15 |
| ATOM | 1983 | CA | SER | 1128 | 54.020 | −2.999 | 8.481 | 1.00 | 44.72 |
| ATOM | 1984 | CB | SER | 1128 | 55.135 | −3.566 | 7.602 | 1.00 | 45.14 |
| ATOM | 1985 | OG | SER | 1128 | 54.630 | −4.527 | 6.687 | 1.00 | 47.68 |
| ATOM | 1986 | C | SER | 1128 | 53.097 | −2.113 | 7.657 | 1.00 | 44.66 |
| ATOM | 1987 | O | SER | 1128 | 53.444 | −0.984 | 7.315 | 1.00 | 44.45 |
| ATOM | 1988 | N | LYS | 1129 | 51.912 | −2.624 | 7.348 | 1.00 | 44.99 |
| ATOM | 1989 | CA | LYS | 1129 | 50.953 | −1.857 | 6.563 | 1.00 | 45.55 |
| ATOM | 1990 | CB | LYS | 1129 | 50.362 | −2.717 | 5.441 | 1.00 | 46.76 |
| ATOM | 1991 | CG | LYS | 1129 | 51.347 | −3.216 | 4.388 | 1.00 | 48.35 |
| ATOM | 1992 | CD | LYS | 1129 | 50.640 | −3.393 | 3.035 | 1.00 | 49.78 |
| ATOM | 1993 | CE | LYS | 1129 | 49.301 | −4.129 | 3.181 | 1.00 | 50.24 |
| ATOM | 1994 | NZ | LYS | 1129 | 48.417 | −3.998 | 1.975 | 1.00 | 50.92 |
| ATOM | 1995 | C | LYS | 1129 | 49.803 | −1.294 | 7.407 | 1.00 | 45.61 |
| ATOM | 1996 | O | LYS | 1129 | 48.876 | −0.686 | 6.868 | 1.00 | 45.44 |
| ATOM | 1997 | N | THR | 1130 | 49.852 | −1.496 | 8.722 | 1.00 | 45.29 |
| ATOM | 1998 | CA | THR | 1130 | 48.790 | −0.991 | 9.584 | 1.00 | 45.00 |
| ATOM | 1999 | CB | THR | 1130 | 48.584 | −1.882 | 10.834 | 1.00 | 45.68 |
| ATOM | 2000 | OG1 | THR | 1130 | 49.787 | −1.911 | 11.610 | 1.00 | 47.25 |
| ATOM | 2001 | OG2 | THR | 1130 | 48.206 | −3.296 | 10.432 | 1.00 | 46.09 |
| ATOM | 2002 | C | THR | 1130 | 49.054 | 0.441 | 10.045 | 1.00 | 44.72 |
| ATOM | 2003 | O | THR | 1130 | 50.152 | 0.973 | 9.879 | 1.00 | 44.85 |
| ATOM | 2004 | N | GLY | 1131 | 48.026 | 1.058 | 10.616 | 1.00 | 44.36 |
| ATOM | 2005 | CA | GLY | 1131 | 48.135 | 2.422 | 11.106 | 1.00 | 43.93 |
| ATOM | 2006 | C | GLY | 1131 | 46.985 | 2.730 | 12.056 | 1.00 | 43.49 |
| ATOM | 2007 | O | GLY | 1131 | 45.998 | 1.986 | 12.085 | 1.00 | 43.28 |
| ATOM | 2008 | N | PRO | 1132 | 47.070 | 3.818 | 12.835 | 1.00 | 43.11 |
| ATOM | 2009 | CD | PRO | 1132 | 48.214 | 4.738 | 12.882 | 1.00 | 43.31 |
| ATOM | 2010 | CA | PRO | 1132 | 46.041 | 4.229 | 13.798 | 1.00 | 42.83 |
| ATOM | 2011 | CB | PRO | 1132 | 46.624 | 5.493 | 14.414 | 1.00 | 43.23 |
| ATOM | 2012 | CG | PRO | 1132 | 48.091 | 5.299 | 14.270 | 1.00 | 43.89 |
| ATOM | 2013 | C | PRO | 1132 | 44.655 | 4.489 | 13.207 | 1.00 | 42.75 |
| ATOM | 2014 | O | PRO | 1132 | 43.638 | 4.168 | 13.827 | 1.00 | 43.21 |
| ATOM | 2015 | N | GLY | 1133 | 44.611 | 5.072 | 12.015 | 1.00 | 41.86 |
| ATOM | 2016 | CA | GLY | 1133 | 43.332 | 5.367 | 11.401 | 1.00 | 40.78 |
| ATOM | 2017 | C | GLY | 1133 | 42.739 | 4.262 | 10.553 | 1.00 | 40.15 |
| ATOM | 2018 | O | GLY | 1133 | 41.987 | 4.540 | 9.621 | 1.00 | 40.24 |
| ATOM | 2019 | N | GLN | 1134 | 43.054 | 3.010 | 10.865 | 1.00 | 39.47 |
| ATOM | 2020 | CA | GLN | 1134 | 42.521 | 1.911 | 10.074 | 1.00 | 38.97 |
| ATOM | 2021 | CB | GLN | 1134 | 43.661 | 1.042 | 9.565 | 1.00 | 39.15 |
| ATOM | 2022 | CG | GLN | 1134 | 44.686 | 1.828 | 8.789 | 1.00 | 39.46 |
| ATOM | 2023 | CD | GLN | 1134 | 45.843 | 0.972 | 8.324 | 1.00 | 40.54 |
| ATOM | 2024 | OE1 | GLN | 1134 | 46.925 | 1.485 | 8.043 | 1.00 | 41.92 |
| ATOM | 2025 | NE2 | GLN | 1134 | 45.622 | −0.337 | 8.232 | 1.00 | 39.45 |
| ATOM | 2026 | C | GLN | 1134 | 41.503 | 1.063 | 10.818 | 1.00 | 38.40 |
| ATOM | 2027 | O | GLN | 1134 | 41.618 | 0.823 | 12.015 | 1.00 | 38.95 |
| ATOM | 2028 | N | LYS | 1135 | 40.499 | 0.622 | 10.080 | 1.00 | 37.75 |
| ATOM | 2029 | CA | LYS | 1135 | 39.421 | −0.202 | 10.600 | 1.00 | 37.02 |
| ATOM | 2030 | CB | LYS | 1135 | 38.305 | −0.194 | 9.555 | 1.00 | 37.54 |
| ATOM | 2031 | CG | LYS | 1135 | 37.062 | −0.998 | 9.839 | 1.00 | 39.30 |
| ATOM | 2032 | CD | LYS | 1135 | 36.083 | −0.706 | 8.710 | 1.00 | 41.83 |
| ATOM | 2033 | CE | LYS | 1135 | 34.860 | −1.603 | 8.713 | 1.00 | 43.72 |
| ATOM | 2034 | NZ | LYS | 1135 | 34.058 | −1.383 | 7.461 | 1.00 | 45.66 |
| ATOM | 2035 | C | LYS | 1135 | 39.915 | −1.628 | 10.880 | 1.00 | 36.31 |
| ATOM | 2036 | O | LYS | 1135 | 39.358 | −2.342 | 11.720 | 1.00 | 36.44 |
| ATOM | 2037 | N | ALA | 1136 | 40.983 | −2.020 | 10.190 | 1.00 | 35.08 |
| ATOM | 2038 | CA | ALA | 1136 | 41.558 | −3.356 | 10.327 | 1.00 | 34.39 |
| ATOM | 2039 | CB | ALA | 1136 | 42.559 | −3.592 | 9.214 | 1.00 | 33.92 |
| ATOM | 2040 | C | ALA | 1136 | 42.212 | −3.679 | 11.674 | 1.00 | 33.83 |
| ATOM | 2041 | O | ALA | 1136 | 42.343 | −4.856 | 12.033 | 1.00 | 33.83 |
| ATOM | 2042 | N | ILE | 1137 | 42.613 | −2.654 | 12.423 | 1.00 | 32.81 |
| ATOM | 2043 | CA | ILE | 1137 | 43.272 | −2.888 | 13.704 | 1.00 | 31.57 |
| ATOM | 2044 | CB | ILE | 1137 | 44.350 | −1.824 | 13.973 | 1.00 | 30.44 |
| ATOM | 2045 | CG2 | ILE | 1137 | 45.144 | −1.561 | 12.709 | 1.00 | 30.36 |
| ATOM | 2046 | CG1 | ILE | 1137 | 43.702 | −0.522 | 14.419 | 1.00 | 29.95 |
| ATOM | 2047 | CD1 | ILE | 1137 | 44.701 | 0.554 | 14.724 | 1.00 | 30.69 |
| ATOM | 2048 | C | ILE | 1137 | 42.322 | −2.922 | 14.903 | 1.00 | 31.51 |
| ATOM | 2049 | O | ILE | 1137 | 42.739 | −3.243 | 16.017 | 1.00 | 31.37 |
| ATOM | 2050 | N | LEU | 1138 | 41.049 | −2.619 | 14.667 | 1.00 | 30.82 |
| ATOM | 2051 | CA | LEU | 1138 | 40.058 | −2.578 | 15.740 | 1.00 | 30.71 |
| ATOM | 2052 | CB | LEU | 1138 | 38.995 | −1.531 | 15.416 | 1.00 | 29.07 |
| ATOM | 2053 | CG | LEU | 1138 | 39.546 | −0.134 | 15.134 | 1.00 | 28.60 |
| ATOM | 2054 | CD1 | LEU | 1138 | 38.432 | 0.782 | 14.630 | 1.00 | 28.90 |
| ATOM | 2055 | CD2 | LEU | 1138 | 40.174 | 0.414 | 16.396 | 1.00 | 27.48 |
| ATOM | 2056 | C | LEU | 1138 | 39.376 | −3.914 | 16.033 | 1.00 | 31.25 |
| ATOM | 2057 | O | LEU | 1138 | 38.825 | −4.563 | 15.136 | 1.00 | 31.68 |
| ATOM | 2058 | N | PHE | 1139 | 39.401 | −4.317 | 17.297 | 1.00 | 31.00 |
| ATOM | 2059 | CA | PHE | 1139 | 38.777 | −5.571 | 17.685 | 1.00 | 31.56 |
| ATOM | 2060 | CB | PHE | 1139 | 39.830 | −6.614 | 18.066 | 1.00 | 32.81 |
| ATOM | 2061 | CG | PHE | 1139 | 40.504 | −7.257 | 16.892 | 1.00 | 33.16 |
| ATOM | 2062 | CD1 | PHE | 1139 | 41.662 | −6.710 | 16.352 | 1.00 | 33.19 |
| ATOM | 2063 | CD2 | PHE | 1139 | 39.972 | −8.412 | 16.323 | 1.00 | 33.35 |
| ATOM | 2064 | CE1 | PHE | 1139 | 42.279 | −7.303 | 15.266 | 1.00 | 32.98 |
| ATOM | 2065 | CE2 | PHE | 1139 | 40.580 | −9.010 | 15.239 | 1.00 | 32.93 |
| ATOM | 2066 | CZ | PHE | 1139 | 41.733 | −8.458 | 14.709 | 1.00 | 32.99 |
| ATOM | 2067 | C | PHE | 1139 | 37.832 | −5.406 | 18.852 | 1.00 | 32.09 |
| ATOM | 2068 | O | PHE | 1139 | 38.091 | −4.644 | 19.792 | 1.00 | 33.11 |
| ATOM | 2069 | N | LEU | 1140 | 36.736 | −6.142 | 18.801 | 1.00 | 31.92 |
| ATOM | 2070 | CA | LEU | 1140 | 35.754 | −6.094 | 19.856 | 1.00 | 32.57 |
| ATOM | 2071 | CB | LEU | 1140 | 34.372 | −5.949 | 19.241 | 1.00 | 32.54 |
| ATOM | 2072 | CG | LEU | 1140 | 33.319 | −5.229 | 20.066 | 1.00 | 33.21 |
| ATOM | 2073 | CD1 | LEU | 1140 | 33.680 | −3.751 | 20.191 | 1.00 | 33.57 |
| ATOM | 2074 | CD2 | LEU | 1140 | 31.977 | −5.382 | 19.389 | 1.00 | 33.32 |
| ATOM | 2075 | C | LEU | 1140 | 35.861 | −7.414 | 20.611 | 1.00 | 33.95 |
| ATOM | 2076 | O | LEU | 1140 | 35.593 | −8.473 | 20.059 | 1.00 | 34.36 |
| ATOM | 2077 | N | PRO | 1141 | 36.280 | −7.370 | 21.883 | 1.00 | 35.11 |
| ATOM | 2078 | CD | PRO | 1141 | 36.668 | −6.195 | 22.685 | 1.00 | 35.49 |
| ATOM | 2079 | CA | PRO | 1141 | 36.405 | −8.601 | 22.665 | 1.00 | 35.94 |
| ATOM | 2080 | CB | PRO | 1141 | 37.214 | −8.149 | 23.868 | 1.00 | 35.97 |
| ATOM | 2081 | CG | PRO | 1141 | 36.686 | −6.755 | 24.088 | 1.00 | 35.79 |
| ATOM | 2082 | C | PRO | 1141 | 35.044 | −9.129 | 23.067 | 1.00 | 37.21 |
| ATOM | 2083 | O | PRO | 1141 | 34.199 | −8.378 | 23.543 | 1.00 | 37.10 |
| ATOM | 2084 | N | MET | 1142 | 34.834 | −10.423 | 22.877 | 1.00 | 39.44 |
| ATOM | 2085 | CA | MET | 1142 | 33.566 | −11.057 | 23.229 | 1.00 | 42.27 |
| ATOM | 2086 | CB | MET | 1142 | 32.776 | −11.425 | 21.972 | 1.00 | 43.78 |
| ATOM | 2087 | CG | MET | 1142 | 32.314 | −10.250 | 21.143 | 1.00 | 46.23 |
| ATOM | 2088 | SD | MET | 1142 | 31.492 | −10.845 | 19.646 | 1.00 | 49.73 |
| ATOM | 2089 | CE | MET | 1142 | 30.201 | −11.895 | 20.358 | 1.00 | 48.43 |
| ATOM | 2090 | C | MET | 1142 | 33.854 | −12.326 | 24.000 | 1.00 | 43.25 |
| ATOM | 2091 | O | MET | 1142 | 34.843 | −13.001 | 23.735 | 1.00 | 43.02 |
| ATOM | 2092 | N | SER | 1143 | 32.986 | −12.655 | 24.945 | 1.00 | 45.28 |
| ATOM | 2093 | CA | SER | 1143 | 33.166 | −13.860 | 25.734 | 1.00 | 48.37 |
| ATOM | 2094 | CB | SER | 1143 | 32.039 | −14.010 | 26.741 | 1.00 | 49.05 |
| ATOM | 2095 | OG | SER | 1143 | 30.921 | −14.630 | 26.123 | 1.00 | 49.26 |
| ATOM | 2096 | C | SER | 1143 | 33.102 | −15.052 | 24.794 | 1.00 | 50.26 |
| ATOM | 2097 | O | SER | 1143 | 32.467 | −14.987 | 23.739 | 1.00 | 50.30 |
| ATOM | 2098 | N | ALA | 1144 | 33.747 | −16.147 | 25.179 | 1.00 | 52.07 |
| ATOM | 2099 | CA | ALA | 1144 | 33.726 | −17.349 | 24.359 | 1.00 | 53.48 |
| ATOM | 2100 | CB | ALA | 1144 | 35.137 | −17.906 | 24.202 | 1.00 | 53.76 |
| ATOM | 2101 | C | ALA | 1144 | 32.814 | −18.381 | 25.014 | 1.00 | 54.18 |
| ATOM | 2102 | O | ALA | 1144 | 33.236 | −18.949 | 26.041 | 1.00 | 55.60 |
| ATOM | 2103 | CB | HIS | 2016 | 30.611 | −12.909 | 53.520 | 1.00 | 49.61 |
| ATOM | 2104 | CG | HIS | 2016 | 29.696 | −13.189 | 52.371 | 1.00 | 52.30 |
| ATOM | 2105 | CD2 | HIS | 2016 | 29.946 | −13.660 | 51.126 | 1.00 | 53.70 |
| ATOM | 2106 | ND1 | HIS | 2016 | 28.342 | −12.933 | 52.420 | 1.00 | 53.21 |
| ATOM | 2107 | CE1 | HIS | 2016 | 27.798 | −13.229 | 51.253 | 1.00 | 53.98 |
| ATOM | 2108 | NE2 | HIS | 2016 | 28.749 | −13.672 | 50.450 | 1.00 | 54.80 |
| ATOM | 2109 | C | HIS | 2016 | 29.064 | −11.546 | 54.916 | 1.00 | 45.96 |
| ATOM | 2110 | O | HIS | 2016 | 28.823 | −12.431 | 55.739 | 1.00 | 45.54 |
| ATOM | 2111 | N | HIS | 2016 | 31.520 | −11.187 | 55.057 | 1.00 | 46.88 |
| ATOM | 2112 | CA | HIS | 2016 | 30.387 | −11.534 | 54.151 | 1.00 | 47.29 |
| ATOM | 2113 | N | PHE | 2017 | 28.211 | −10.569 | 54.627 | 1.00 | 44.46 |
| ATOM | 2114 | CA | PHE | 2017 | 26.924 | −10.430 | 55.296 | 1.00 | 43.76 |
| ATOM | 2115 | CB | PHE | 2017 | 26.086 | −9.384 | 54.559 | 1.00 | 42.17 |
| ATOM | 2116 | CG | PHE | 2017 | 25.565 | −9.860 | 53.242 | 1.00 | 41.19 |
| ATOM | 2117 | CD1 | PHE | 2017 | 24.281 | −10.383 | 53.138 | 1.00 | 40.67 |
| ATOM | 2118 | CD2 | PHE | 2017 | 26.367 | −9.824 | 52.112 | 1.00 | 40.83 |
| ATOM | 2119 | CE1 | PHE | 2017 | 23.802 | −10.867 | 51.928 | 1.00 | 40.69 |
| ATOM | 2120 | CE2 | PHE | 2017 | 25.898 | −10.306 | 50.893 | 1.00 | 41.19 |
| ATOM | 2121 | CZ | PHE | 2017 | 24.610 | −10.831 | 50.801 | 1.00 | 40.82 |
| ATOM | 2122 | C | PHE | 2017 | 26.140 | −11.748 | 55.417 | 1.00 | 43.77 |
| ATOM | 2123 | O | PHE | 2017 | 25.388 | −11.941 | 56.381 | 1.00 | 43.74 |
| ATOM | 2124 | N | LYS | 2018 | 26.317 | −12.654 | 54.453 | 1.00 | 43.45 |
| ATOM | 2125 | CA | LYS | 2018 | 25.605 | −13.934 | 54.477 | 1.00 | 43.23 |
| ATOM | 2126 | CB | LYS | 2018 | 25.793 | −14.689 | 53.157 | 1.00 | 43.20 |
| ATOM | 2127 | C | LYS | 2018 | 26.025 | −14.837 | 55.634 | 1.00 | 43.00 |
| ATOM | 2128 | O | LYS | 2018 | 25.197 | −15.563 | 56.182 | 1.00 | 43.52 |
| ATOM | 2129 | N | ASP | 2019 | 27.302 | −14.783 | 56.006 | 1.00 | 42.35 |
| ATOM | 2130 | CA | ASP | 2019 | 27.822 | −15.613 | 57.081 | 1.00 | 41.21 |
| ATOM | 2131 | CB | ASP | 2019 | 29.348 | −15.517 | 57.131 | 1.00 | 42.88 |
| ATOM | 2132 | CG | ASP | 2019 | 30.006 | −15.924 | 55.819 | 1.00 | 44.66 |
| ATOM | 2133 | OD1 | ASP | 2019 | 29.463 | −16.807 | 55.107 | 1.00 | 45.47 |
| ATOM | 2134 | OD2 | ASP | 2019 | 31.080 | −15.368 | 55.506 | 1.00 | 45.66 |
| ATOM | 2135 | C | ASP | 2019 | 27.236 | −15.268 | 58.443 | 1.00 | 40.14 |
| ATOM | 2136 | O | ASP | 2019 | 26.659 | −14.194 | 58.636 | 1.00 | 39.64 |
| ATOM | 2137 | N | PRO | 2020 | 27.359 | −16.197 | 59.408 | 1.00 | 39.24 |
| ATOM | 2138 | CD | PRO | 2020 | 27.900 | −17.555 | 59.242 | 1.00 | 38.60 |
| ATOM | 2139 | CA | PRO | 2020 | 26.848 | −16.006 | 60.769 | 1.00 | 37.92 |
| ATOM | 2140 | CB | PRO | 2020 | 26.930 | −17.406 | 61.380 | 1.00 | 37.81 |
| ATOM | 2141 | CG | PRO | 2020 | 27.044 | −18.325 | 60.180 | 1.00 | 38.70 |
| ATOM | 2142 | C | PRO | 2020 | 27.761 | −15.029 | 61.494 | 1.00 | 36.76 |
| ATOM | 2143 | O | PRO | 2020 | 28.916 | −14.851 | 61.109 | 1.00 | 36.56 |
| ATOM | 2144 | N | LYS | 2021 | 27.251 | −14.406 | 62.542 | 1.00 | 35.46 |
| ATOM | 2145 | CA | LYS | 2021 | 28.049 | −13.462 | 63.292 | 1.00 | 35.36 |
| ATOM | 2146 | CB | LYS | 2021 | 27.651 | −12.026 | 62.938 | 1.00 | 36.14 |
| ATOM | 2147 | CG | LYS | 2021 | 27.749 | −11.680 | 61.481 | 1.00 | 37.36 |
| ATOM | 2148 | CD | LYS | 2021 | 27.412 | −10.220 | 61.259 | 1.00 | 38.89 |
| ATOM | 2149 | CE | LYS | 2021 | 27.502 | −9.864 | 59.778 | 1.00 | 40.69 |
| ATOM | 2150 | NZ | LYS | 2021 | 27.147 | −8.439 | 59.506 | 1.00 | 41.77 |
| ATOM | 2151 | C | LYS | 2021 | 27.847 | −13.659 | 64.779 | 1.00 | 34.94 |
| ATOM | 2152 | O | LYS | 2021 | 26.868 | −14.274 | 65.208 | 1.00 | 33.96 |
| ATOM | 2153 | N | ARG | 2022 | 28.797 | −13.154 | 65.562 | 1.00 | 34.36 |
| ATOM | 2154 | CA | ARG | 2022 | 28.678 | −13.198 | 67.016 | 1.00 | 33.66 |
| ATOM | 2155 | CB | ARG | 2022 | 29.947 | −13.709 | 67.699 | 1.00 | 34.43 |
| ATOM | 2156 | CG | ARG | 2022 | 30.477 | −15.049 | 67.231 | 1.00 | 36.74 |
| ATOM | 2157 | CD | ARG | 2022 | 31.768 | −15.333 | 67.986 | 1.00 | 39.41 |
| ATOM | 2158 | NE | ARG | 2022 | 32.738 | −16.138 | 67.243 | 1.00 | 42.21 |
| ATOM | 2159 | CZ | ARG | 2022 | 32.607 | −17.436 | 66.987 | 1.00 | 43.42 |
| ATOM | 2160 | NH1 | ARG | 2022 | 31.534 | −18.103 | 67.406 | 1.00 | 44.07 |
| ATOM | 2161 | NH2 | ARG | 2022 | 33.572 | −18.076 | 66.340 | 1.00 | 43.95 |
| ATOM | 2162 | C | ARG | 2022 | 28.470 | −11.734 | 67.401 | 1.00 | 32.41 |
| ATOM | 2163 | O | ARG | 2022 | 29.052 | −10.831 | 66.795 | 1.00 | 31.75 |
| ATOM | 2164 | N | LEU | 2023 | 27.618 | −11.491 | 68.382 | 1.00 | 31.14 |
| ATOM | 2165 | CA | LEU | 2023 | 27.394 | −10.132 | 68.824 | 1.00 | 30.16 |
| ATOM | 2166 | CB | LEU | 2023 | 25.903 | −9.797 | 68.835 | 1.00 | 29.73 |
| ATOM | 2167 | CG | LEU | 2023 | 25.329 | −9.759 | 67.409 | 1.00 | 30.46 |
| ATOM | 2168 | CD1 | LEU | 2023 | 23.839 | −9.475 | 67.446 | 1.00 | 29.76 |
| ATOM | 2169 | CD2 | LEU | 2023 | 26.067 | −8.694 | 66.579 | 1.00 | 29.94 |
| ATOM | 2170 | C | LEU | 2023 | 28.014 | −9.962 | 70.194 | 1.00 | 29.98 |
| ATOM | 2171 | O | LEU | 2023 | 27.520 | −10.476 | 71.198 | 1.00 | 29.93 |
| ATOM | 2172 | N | TYR | 2024 | 29.134 | −9.251 | 70.199 | 1.00 | 29.87 |
| ATOM | 2173 | CA | TYR | 2024 | 29.909 | −8.977 | 71.398 | 1.00 | 30.34 |
| ATOM | 2174 | CB | TYR | 2024 | 31.378 | −8.856 | 70.987 | 1.00 | 30.88 |
| ATOM | 2175 | CG | TYR | 2024 | 32.357 | −8.439 | 72.054 | 1.00 | 31.94 |
| ATOM | 2176 | CD1 | TYR | 2024 | 32.447 | −7.109 | 72.472 | 1.00 | 31.98 |
| ATOM | 2177 | CE1 | TYR | 2024 | 33.417 | −6.714 | 73.385 | 1.00 | 31.90 |
| ATOM | 2178 | CD2 | TYR | 2024 | 33.256 | −9.359 | 72.589 | 1.00 | 31.81 |
| ATOM | 2179 | CE2 | TYR | 2024 | 34.218 | −8.972 | 73.498 | 1.00 | 31.40 |
| ATOM | 2180 | CZ | TYR | 2024 | 34.297 | −7.655 | 73.887 | 1.00 | 31.83 |
| ATOM | 2181 | OH | TYR | 2024 | 35.277 | −7.281 | 74.771 | 1.00 | 32.59 |
| ATOM | 2182 | C | TYR | 2024 | 29.386 | −7.697 | 72.038 | 1.00 | 30.32 |
| ATOM | 2183 | O | TYR | 2024 | 29.433 | −6.632 | 71.441 | 1.00 | 31.34 |
| ATOM | 2184 | N | CYS | 2025 | 28.871 | −7.816 | 73.253 | 1.00 | 30.54 |
| ATOM | 2185 | CA | CYS | 2025 | 28.327 | −6.682 | 73.963 | 1.00 | 30.56 |
| ATOM | 2186 | CB | CYS | 2025 | 27.377 | −7.163 | 75.037 | 1.00 | 30.66 |
| ATOM | 2187 | SG | CYS | 2025 | 26.531 | −5.801 | 75.813 | 1.00 | 32.95 |
| ATOM | 2188 | C | CYS | 2025 | 29.417 | −5.847 | 74.603 | 1.00 | 31.07 |
| ATOM | 2189 | O | CYS | 2025 | 30.300 | −6.380 | 75.274 | 1.00 | 31.19 |
| ATOM | 2190 | N | LYS | 2026 | 29.347 | −4.532 | 74.414 | 1.00 | 31.54 |
| ATOM | 2191 | CA | LYS | 2026 | 30.354 | −3.638 | 74.979 | 1.00 | 31.91 |
| ATOM | 2192 | CB | LYS | 2026 | 30.147 | −2.216 | 74.465 | 1.00 | 30.14 |
| ATOM | 2193 | CG | LYS | 2026 | 31.273 | −1.256 | 74.794 | 1.00 | 28.90 |
| ATOM | 2194 | CD | LYS | 2026 | 31.025 | 0.107 | 74.149 | 1.00 | 27.04 |
| ATOM | 2195 | CE | LYS | 2026 | 32.193 | 1.058 | 74.354 | 1.00 | 26.12 |
| ATOM | 2196 | NZ | LYS | 2026 | 32.269 | 1.603 | 75.735 | 1.00 | 27.32 |
| ATOM | 2197 | C | LYS | 2026 | 30.302 | −3.653 | 76.504 | 1.00 | 33.45 |
| ATOM | 2198 | O | LYS | 2026 | 31.294 | −3.375 | 77.174 | 1.00 | 33.52 |
| ATOM | 2199 | N | ASN | 2027 | 29.145 | −4.006 | 77.049 | 1.00 | 34.56 |
| ATOM | 2200 | CA | ASN | 2027 | 28.966 | −4.038 | 78.490 | 1.00 | 35.65 |
| ATOM | 2201 | CB | ASN | 2027 | 27.493 | −3.774 | 78.799 | 1.00 | 36.90 |
| ATOM | 2202 | CG | ASN | 2027 | 27.222 | −3.626 | 80.275 | 1.00 | 38.46 |
| ATOM | 2203 | OD1 | ASN | 2027 | 27.992 | −2.994 | 81.004 | 1.00 | 39.90 |
| ATOM | 2204 | ND2 | ASN | 2027 | 26.108 | −4.193 | 80.725 | 1.00 | 38.80 |
| ATOM | 2205 | C | ASN | 2027 | 29.433 | −5.350 | 79.132 | 1.00 | 36.07 |
| ATOM | 2206 | O | ASN | 2027 | 28.626 | −6.235 | 79.422 | 1.00 | 37.21 |
| ATOM | 2207 | N | GLY | 2028 | 30.740 | −5.481 | 79.338 | 1.00 | 35.53 |
| ATOM | 2208 | CA | GLY | 2028 | 31.257 | −6.681 | 79.961 | 1.00 | 34.90 |
| ATOM | 2209 | C | GLY | 2028 | 31.912 | −7.658 | 79.008 | 1.00 | 35.77 |
| ATOM | 2210 | O | GLY | 2028 | 32.597 | −8.581 | 79.440 | 1.00 | 36.15 |
| ATOM | 2211 | N | GLY | 2029 | 31.705 | −7.469 | 77.711 | 1.00 | 35.59 |
| ATOM | 2212 | CA | GLY | 2029 | 32.306 | −8.367 | 76.745 | 1.00 | 35.25 |
| ATOM | 2213 | C | GLY | 2029 | 31.611 | −9.715 | 76.629 | 1.00 | 35.54 |
| ATOM | 2214 | O | GLY | 2029 | 32.254 | −10.732 | 76.336 | 1.00 | 35.83 |
| ATOM | 2215 | N | PHE | 2030 | 30.301 | −9.731 | 76.861 | 1.00 | 34.61 |
| ATOM | 2216 | CA | PHE | 2030 | 29.523 | −10.955 | 76.764 | 1.00 | 34.19 |
| ATOM | 2217 | CB | PHE | 2030 | 28.398 | −10.951 | 77.798 | 1.00 | 34.27 |
| ATOM | 2218 | CG | PHE | 2030 | 28.875 | −11.031 | 79.214 | 1.00 | 35.00 |
| ATOM | 2219 | CD1 | PHE | 2030 | 29.170 | −9.878 | 79.933 | 1.00 | 35.78 |
| ATOM | 2220 | CD2 | PHE | 2030 | 29.047 | −12.264 | 79.831 | 1.00 | 34.75 |
| ATOM | 2221 | CE1 | PHE | 2030 | 29.633 | −9.959 | 81.250 | 1.00 | 35.19 |
| ATOM | 2222 | CE2 | PHE | 2030 | 29.510 | −12.349 | 81.145 | 1.00 | 34.33 |
| ATOM | 2223 | CZ | PHE | 2030 | 29.801 | −11.197 | 81.851 | 1.00 | 34.60 |
| ATOM | 2224 | C | PHE | 2030 | 28.908 | −11.097 | 75.370 | 1.00 | 33.93 |
| ATOM | 2225 | O | PHE | 2030 | 28.359 | −10.136 | 74.830 | 1.00 | 33.62 |
| ATOM | 2226 | N | PHE | 2031 | 29.007 | −12.295 | 74.801 | 1.00 | 32.96 |
| ATOM | 2227 | CA | PHE | 2031 | 28.435 | −12.591 | 73.489 | 1.00 | 32.61 |
| ATOM | 2228 | CB | PHE | 2031 | 29.122 | −13.804 | 72.866 | 1.00 | 30.62 |
| ATOM | 2229 | CG | PHE | 2031 | 30.474 | −13.514 | 72.317 | 1.00 | 28.88 |
| ATOM | 2230 | CD1 | PHE | 2031 | 30.614 | −12.801 | 71.139 | 1.00 | 28.57 |
| ATOM | 2231 | CD2 | PHE | 2031 | 31.616 | −13.947 | 72.981 | 1.00 | 28.17 |
| ATOM | 2232 | CE1 | PHE | 2031 | 31.878 | −12.525 | 70.624 | 1.00 | 28.68 |
| ATOM | 2233 | CE2 | PHE | 2031 | 32.884 | −13.676 | 72.475 | 1.00 | 27.81 |
| ATOM | 2234 | CZ | PHE | 2031 | 33.017 | −12.966 | 71.297 | 1.00 | 27.69 |
| ATOM | 2235 | C | PHE | 2031 | 26.970 | −12.926 | 73.677 | 1.00 | 32.61 |
| ATOM | 2236 | O | PHE | 2031 | 26.625 | −13.639 | 74.610 | 1.00 | 33.68 |
| ATOM | 2237 | N | LEU | 2032 | 26.111 | −12.413 | 72.807 | 1.00 | 32.82 |
| ATOM | 2238 | CA | LEU | 2032 | 24.694 | −12.712 | 72.924 | 1.00 | 32.89 |
| ATOM | 2239 | CB | LEU | 2032 | 23.881 | −11.873 | 71.937 | 1.00 | 30.95 |
| ATOM | 2240 | CG | LEU | 2032 | 22.373 | −12.077 | 72.055 | 1.00 | 30.07 |
| ATOM | 2241 | CD1 | LEU | 2032 | 21.924 | −11.664 | 73.444 | 1.00 | 29.16 |
| ATOM | 2242 | CD2 | LEU | 2032 | 21.652 | −11.278 | 70.990 | 1.00 | 28.97 |
| ATOM | 2243 | C | LEU | 2032 | 24.514 | −14.203 | 72.633 | 1.00 | 33.63 |
| ATOM | 2244 | O | LEU | 2032 | 24.999 | −14.718 | 71.618 | 1.00 | 33.71 |
| ATOM | 2245 | N | ARG | 2033 | 23.835 | −14.903 | 73.536 | 1.00 | 34.59 |
| ATOM | 2246 | CA | ARG | 2033 | 23.618 | −16.329 | 73.362 | 1.00 | 35.19 |
| ATOM | 2247 | CB | ARG | 2033 | 24.372 | −17.111 | 74.421 | 1.00 | 34.72 |
| ATOM | 2248 | CG | ARG | 2033 | 24.274 | −18.618 | 74.244 | 1.00 | 33.31 |
| ATOM | 2249 | CD | ARG | 2033 | 25.141 | −19.334 | 75.257 | 1.00 | 31.04 |
| ATOM | 2250 | NE | ARG | 2033 | 24.681 | −19.100 | 76.616 | 1.00 | 29.30 |
| ATOM | 2251 | CZ | ARG | 2033 | 25.231 | −19.668 | 77.681 | 1.00 | 29.82 |
| ATOM | 2252 | NH1 | ARG | 2033 | 26.257 | −20.493 | 77.521 | 1.00 | 29.55 |
| ATOM | 2253 | NH2 | ARG | 2033 | 24.761 | −19.417 | 78.897 | 1.00 | 29.44 |
| ATOM | 2254 | C | ARG | 2033 | 22.164 | −16.762 | 73.375 | 1.00 | 36.20 |
| ATOM | 2255 | O | ARG | 2033 | 21.380 | −16.371 | 74.246 | 1.00 | 36.24 |
| ATOM | 2256 | N | ILE | 2034 | 21.815 | −17.572 | 72.385 | 1.00 | 37.13 |
| ATOM | 2257 | CA | ILE | 2034 | 20.473 | −18.098 | 72.266 | 1.00 | 38.70 |
| ATOM | 2258 | CB | ILE | 2034 | 19.895 | −17.795 | 70.884 | 1.00 | 38.45 |
| ATOM | 2259 | CG2 | ILE | 2034 | 18.493 | −18.372 | 70.777 | 1.00 | 37.91 |
| ATOM | 2260 | CG1 | ILE | 2034 | 19.891 | −16.281 | 70.667 | 1.00 | 37.72 |
| ATOM | 2261 | CD1 | ILE | 2034 | 19.396 | −15.847 | 69.313 | 1.00 | 37.52 |
| ATOM | 2262 | C | ILE | 2034 | 20.544 | −19.602 | 72.510 | 1.00 | 39.99 |
| ATOM | 2263 | O | ILE | 2034 | 21.110 | −20.351 | 71.706 | 1.00 | 39.24 |
| ATOM | 2264 | N | HIS | 2035 | 19.993 | −20.037 | 73.640 | 1.00 | 41.62 |
| ATOM | 2265 | CA | HIS | 2035 | 20.043 | −21.450 | 73.953 | 1.00 | 43.67 |
| ATOM | 2266 | CB | HIS | 2035 | 20.042 | −21.692 | 75.458 | 1.00 | 44.52 |
| ATOM | 2267 | CG | HIS | 2035 | 20.808 | −22.915 | 75.857 | 1.00 | 45.32 |
| ATOM | 2268 | CD2 | HIS | 2035 | 22.124 | −23.092 | 76.131 | 1.00 | 45.43 |
| ATOM | 2269 | ND1 | HIS | 2035 | 20.227 | −24.166 | 75.944 | 1.00 | 45.72 |
| ATOM | 2270 | CE1 | HIS | 2035 | 21.154 | −25.054 | 76.253 | 1.00 | 45.87 |
| ATOM | 2271 | NE2 | HIS | 2035 | 22.314 | −24.432 | 76.372 | 1.00 | 44.96 |
| ATOM | 2272 | C | HIS | 2035 | 18.939 | −22.240 | 73.282 | 1.00 | 44.91 |
| ATOM | 2273 | O | HIS | 2035 | 17.849 | −21.720 | 73.002 | 1.00 | 44.68 |
| ATOM | 2274 | N | PRO | 2036 | 19.236 | −23.508 | 72.956 | 1.00 | 46.21 |
| ATOM | 2275 | CD | PRO | 2036 | 20.598 | −24.078 | 72.880 | 1.00 | 46.46 |
| ATOM | 2276 | CA | PRO | 2036 | 18.278 | −24.396 | 72.305 | 1.00 | 47.15 |
| ATOM | 2277 | CB | PRO | 2036 | 18.994 | −25.730 | 72.349 | 1.00 | 46.84 |
| ATOM | 2278 | CG | PRO | 2036 | 20.398 | −25.309 | 72.016 | 1.00 | 46.49 |
| ATOM | 2279 | C | PRO | 2036 | 16.902 | −24.422 | 72.959 | 1.00 | 48.40 |
| ATOM | 2280 | O | PRO | 2036 | 15.885 | −24.565 | 72.272 | 1.00 | 48.57 |
| ATOM | 2281 | N | ASP | 2037 | 16.862 | −24.256 | 74.278 | 1.00 | 49.58 |
| ATOM | 2282 | CA | ASP | 2037 | 15.591 | −24.277 | 74.995 | 1.00 | 51.03 |
| ATOM | 2283 | CB | ASP | 2037 | 15.820 | −24.763 | 76.426 | 1.00 | 52.08 |
| ATOM | 2284 | CG | ASP | 2037 | 16.492 | −23.729 | 77.288 | 1.00 | 52.53 |
| ATOM | 2285 | OD1 | ASP | 2037 | 15.777 | −22.859 | 77.826 | 1.00 | 52.62 |
| ATOM | 2286 | OD2 | ASP | 2037 | 17.734 | −23.785 | 77.419 | 1.00 | 53.74 |
| ATOM | 2287 | C | ASP | 2037 | 14.840 | −22.939 | 75.019 | 1.00 | 51.65 |
| ATOM | 2288 | O | ASP | 2037 | 13.784 | −22.826 | 75.653 | 1.00 | 51.96 |
| ATOM | 2289 | N | GLY | 2038 | 15.373 | −21.925 | 74.341 | 1.00 | 51.93 |
| ATOM | 2290 | CA | GLY | 2038 | 14.697 | −20.633 | 74.314 | 1.00 | 51.28 |
| ATOM | 2291 | C | GLY | 2038 | 15.226 | −19.586 | 75.283 | 1.00 | 50.66 |
| ATOM | 2292 | O | GLY | 2038 | 14.718 | −18.465 | 75.337 | 1.00 | 50.54 |
| ATOM | 2293 | N | ARG | 2039 | 16.246 | −19.953 | 76.051 | 1.00 | 49.81 |
| ATOM | 2294 | CA | ARG | 2039 | 16.870 | −19.053 | 77.016 | 1.00 | 49.30 |
| ATOM | 2295 | CB | ARG | 2039 | 17.567 | −19.888 | 78.089 | 1.00 | 50.37 |
| ATOM | 2296 | CG | ARG | 2039 | 18.261 | −19.100 | 79.195 | 1.00 | 52.28 |
| ATOM | 2297 | CD | ARG | 2039 | 19.172 | −20.028 | 80.013 | 1.00 | 53.80 |
| ATOM | 2298 | NE | ARG | 2039 | 18.996 | −21.422 | 79.609 | 1.00 | 55.19 |
| ATOM | 2299 | CZ | ARG | 2039 | 19.874 | −22.399 | 79.828 | 1.00 | 55.99 |
| ATOM | 2300 | NH1 | ARG | 2039 | 19.597 | −23.634 | 79.412 | 1.00 | 55.66 |
| ATOM | 2301 | NH2 | ARG | 2039 | 21.024 | −22.149 | 80.457 | 1.00 | 55.06 |
| ATOM | 2302 | C | ARG | 2039 | 17.901 | −18.136 | 76.337 | 1.00 | 48.32 |
| ATOM | 2303 | O | ARG | 2039 | 18.741 | −18.599 | 75.561 | 1.00 | 48.18 |
| ATOM | 2304 | N | VAL | 2040 | 17.841 | −16.843 | 76.646 | 1.00 | 46.76 |
| ATOM | 2305 | CA | VAL | 2040 | 18.750 | −15.852 | 76.073 | 1.00 | 45.02 |
| ATOM | 2306 | CB | VAL | 2040 | 17.945 | −14.753 | 75.332 | 1.00 | 44.80 |
| ATOM | 2307 | CG1 | VAL | 2040 | 18.876 | −13.701 | 74.769 | 1.00 | 44.89 |
| ATOM | 2308 | CG2 | VAL | 2040 | 17.116 | −15.368 | 74.222 | 1.00 | 45.45 |
| ATOM | 2309 | C | VAL | 2040 | 19.599 | −15.177 | 77.159 | 1.00 | 44.01 |
| ATOM | 2310 | O | VAL | 2040 | 19.065 | −14.634 | 78.122 | 1.00 | 43.37 |
| ATOM | 2311 | N | ASP | 2041 | 20.915 | −15.214 | 77.001 | 1.00 | 42.82 |
| ATOM | 2312 | CA | ASP | 2041 | 21.825 | −14.589 | 77.955 | 1.00 | 42.01 |
| ATOM | 2313 | CB | ASP | 2041 | 22.060 | −15.505 | 79.165 | 1.00 | 41.22 |
| ATOM | 2314 | CG | ASP | 2041 | 22.701 | −16.845 | 78.788 | 1.00 | 41.03 |
| ATOM | 2315 | OD1 | ASP | 2041 | 22.959 | −17.655 | 79.703 | 1.00 | 39.70 |
| ATOM | 2316 | OD2 | ASP | 2041 | 22.944 | −17.100 | 77.587 | 1.00 | 40.84 |
| ATOM | 2317 | C | ASP | 2041 | 23.153 | −14.295 | 77.266 | 1.00 | 41.88 |
| ATOM | 2318 | O | ASP | 2041 | 23.240 | −14.320 | 76.042 | 1.00 | 41.39 |
| ATOM | 2319 | N | GLY | 2042 | 24.188 | −14.027 | 78.053 | 1.00 | 41.66 |
| ATOM | 2320 | CA | GLY | 2042 | 25.487 | −13.749 | 77.474 | 1.00 | 41.26 |
| ATOM | 2321 | C | GLY | 2042 | 26.597 | −14.582 | 78.082 | 1.00 | 41.38 |
| ATOM | 2322 | O | GLY | 2042 | 26.492 | −15.042 | 79.216 | 1.00 | 41.29 |
| ATOM | 2323 | N | VAL | 2043 | 27.657 | −14.793 | 77.312 | 1.00 | 41.06 |
| ATOM | 2324 | CA | VAL | 2043 | 28.809 | −15.549 | 77.785 | 1.00 | 41.22 |
| ATOM | 2325 | CB | VAL | 2043 | 28.736 | −17.056 | 77.425 | 1.00 | 40.97 |
| ATOM | 2326 | CG1 | VAL | 2043 | 27.638 | −17.723 | 78.210 | 1.00 | 41.41 |
| ATOM | 2327 | CG2 | VAL | 2043 | 28.509 | −17.232 | 75.945 | 1.00 | 40.78 |
| ATOM | 2328 | C | VAL | 2043 | 30.046 | −14.966 | 77.147 | 1.00 | 41.51 |
| ATOM | 2329 | O | VAL | 2043 | 29.979 | −14.392 | 76.066 | 1.00 | 41.82 |
| ATOM | 2330 | N | ARG | 2044 | 31.178 | −15.123 | 77.817 | 1.00 | 41.88 |
| ATOM | 2331 | CA | ARG | 2044 | 32.435 | −14.594 | 77.317 | 1.00 | 42.03 |
| ATOM | 2332 | CB | ARG | 2044 | 33.333 | −14.193 | 78.479 | 1.00 | 40.02 |
| ATOM | 2333 | CG | ARG | 2044 | 32.804 | −13.049 | 79.306 | 1.00 | 39.81 |
| ATOM | 2334 | CD | ARG | 2044 | 33.912 | −12.503 | 80.177 | 1.00 | 39.56 |
| ATOM | 2335 | NE | ARG | 2044 | 33.585 | −11.194 | 80.719 | 1.00 | 40.08 |
| ATOM | 2336 | CZ | ARG | 2044 | 33.180 | −10.980 | 81.964 | 1.00 | 40.53 |
| ATOM | 2337 | NH1 | ARG | 2044 | 33.053 | −11.993 | 82.806 | 1.00 | 40.71 |
| ATOM | 2338 | NH2 | ARG | 2044 | 32.899 | −9.750 | 82.361 | 1.00 | 40.74 |
| ATOM | 2339 | C | ARG | 2044 | 33.198 | −15.556 | 76.428 | 1.00 | 43.22 |
| ATOM | 2340 | O | ARG | 2044 | 33.965 | −15.133 | 75.575 | 1.00 | 43.47 |
| ATOM | 2341 | N | GLU | 2045 | 32.990 | −16.850 | 76.627 | 1.00 | 45.09 |
| ATOM | 2342 | CA | GLU | 2045 | 33.698 | −17.855 | 75.848 | 1.00 | 46.93 |
| ATOM | 2343 | CB | GLU | 2045 | 33.418 | −19.245 | 76.427 | 1.00 | 48.97 |
| ATOM | 2344 | CG | GLU | 2045 | 34.236 | −20.359 | 75.784 | 1.00 | 53.16 |
| ATOM | 2345 | CD | GLU | 2045 | 34.293 | −21.622 | 76.645 | 1.00 | 56.41 |
| ATOM | 2346 | OE1 | GLU | 2045 | 33.213 | −22.140 | 77.029 | 1.00 | 57.88 |
| ATOM | 2347 | OE2 | GLU | 2045 | 35.422 | −22.099 | 76.934 | 1.00 | 57.40 |
| ATOM | 2348 | C | GLU | 2045 | 33.342 | −17.806 | 74.366 | 1.00 | 46.73 |
| ATOM | 2349 | O | GLU | 2045 | 32.239 | −18.162 | 73.967 | 1.00 | 47.26 |
| ATOM | 2350 | N | LYS | 2046 | 34.300 | −17.373 | 73.559 | 1.00 | 46.80 |
| ATOM | 2351 | CA | LYS | 2046 | 34.135 | −17.243 | 72.116 | 1.00 | 47.28 |
| ATOM | 2352 | CB | LYS | 2046 | 35.438 | −16.690 | 71.525 | 1.00 | 47.56 |
| ATOM | 2353 | CG | LYS | 2046 | 35.419 | −16.385 | 70.038 | 1.00 | 48.68 |
| ATOM | 2354 | CD | LYS | 2046 | 36.739 | −15.746 | 69.598 | 1.00 | 50.33 |
| ATOM | 2355 | CE | LYS | 2046 | 36.754 | −15.372 | 68.105 | 1.00 | 51.42 |
| ATOM | 2356 | NZ | LYS | 2046 | 36.885 | −16.552 | 67.186 | 1.00 | 52.03 |
| ATOM | 2357 | C | LYS | 2046 | 33.768 | −18.553 | 71.416 | 1.00 | 47.43 |
| ATOM | 2358 | O | LYS | 2046 | 33.174 | −18.545 | 70.333 | 1.00 | 47.26 |
| ATOM | 2359 | N | SER | 2047 | 34.117 | −19.674 | 72.040 | 1.00 | 47.72 |
| ATOM | 2360 | CA | SER | 2047 | 33.870 | −20.992 | 71.463 | 1.00 | 47.53 |
| ATOM | 2361 | CB | SER | 2047 | 34.941 | −21.977 | 71.938 | 1.00 | 48.08 |
| ATOM | 2362 | OG | SER | 2047 | 34.991 | −22.027 | 73.352 | 1.00 | 48.74 |
| ATOM | 2363 | C | SER | 2047 | 32.497 | −21.577 | 71.752 | 1.00 | 47.41 |
| ATOM | 2364 | O | SER | 2047 | 32.152 | −22.641 | 71.234 | 1.00 | 47.44 |
| ATOM | 2365 | N | ASP | 2048 | 31.716 | −20.900 | 72.584 | 1.00 | 46.97 |
| ATOM | 2366 | CA | ASP | 2048 | 30.380 | −21.386 | 72.895 | 1.00 | 46.42 |
| ATOM | 2367 | CB | ASP | 2048 | 29.640 | −20.361 | 73.748 | 1.00 | 46.81 |
| ATOM | 2368 | CG | ASP | 2048 | 28.385 | −20.926 | 74.385 | 1.00 | 47.58 |
| ATOM | 2369 | OD1 | ASP | 2048 | 28.432 | −21.294 | 75.584 | 1.00 | 47.38 |
| ATOM | 2370 | OD2 | ASP | 2048 | 27.355 | −21.008 | 73.680 | 1.00 | 47.47 |
| ATOM | 2371 | C | ASP | 2048 | 29.648 | −21.592 | 71.562 | 1.00 | 46.09 |
| ATOM | 2372 | O | ASP | 2048 | 29.691 | −20.739 | 70.677 | 1.00 | 45.80 |
| ATOM | 2373 | N | PRO | 2049 | 28.966 | −22.730 | 71.401 | 1.00 | 45.72 |
| ATOM | 2374 | CD | PRO | 2049 | 28.870 | −23.861 | 72.340 | 1.00 | 44.97 |
| ATOM | 2375 | CA | PRO | 2049 | 28.243 | −23.016 | 70.157 | 1.00 | 44.93 |
| ATOM | 2376 | CB | PRO | 2049 | 27.995 | −24.512 | 70.258 | 1.00 | 45.13 |
| ATOM | 2377 | CG | PRO | 2049 | 27.757 | −24.681 | 71.738 | 1.00 | 45.31 |
| ATOM | 2378 | C | PRO | 2049 | 26.943 | −22.253 | 69.934 | 1.00 | 43.83 |
| ATOM | 2379 | O | PRO | 2049 | 26.490 | −22.116 | 68.799 | 1.00 | 44.32 |
| ATOM | 2380 | N | HIS | 2050 | 26.350 | −21.753 | 71.009 | 1.00 | 42.36 |
| ATOM | 2381 | CA | HIS | 2050 | 25.078 | −21.052 | 70.909 | 1.00 | 41.48 |
| ATOM | 2382 | CB | HIS | 2050 | 24.216 | −21.433 | 72.112 | 1.00 | 41.65 |
| ATOM | 2383 | CG | HIS | 2050 | 24.257 | −22.897 | 72.438 | 1.00 | 43.12 |
| ATOM | 2384 | CD2 | HIS | 2050 | 24.671 | −23.553 | 73.550 | 1.00 | 42.95 |
| ATOM | 2385 | ND1 | HIS | 2050 | 23.873 | −23.873 | 71.543 | 1.00 | 43.47 |
| ATOM | 2386 | CE1 | HIS | 2050 | 24.051 | −25.065 | 72.088 | 1.00 | 42.38 |
| ATOM | 2387 | NE2 | HIS | 2050 | 24.534 | −24.898 | 73.306 | 1.00 | 41.45 |
| ATOM | 2388 | C | HIS | 2050 | 25.153 | −19.528 | 70.766 | 1.00 | 40.56 |
| ATOM | 2389 | O | HIS | 2050 | 24.185 | −18.829 | 71.070 | 1.00 | 39.83 |
| ATOM | 2390 | N | ILE | 2051 | 26.291 | −19.012 | 70.304 | 1.00 | 39.16 |
| ATOM | 2391 | CA | ILE | 2051 | 26.429 | −17.567 | 70.126 | 1.00 | 38.23 |
| ATOM | 2392 | CB | ILE | 2051 | 27.631 | −16.990 | 70.912 | 1.00 | 37.53 |
| ATOM | 2393 | CG2 | ILE | 2051 | 27.443 | −17.236 | 72.399 | 1.00 | 36.56 |
| ATOM | 2394 | CG1 | ILE | 2051 | 28.932 | −17.613 | 70.415 | 1.00 | 37.16 |
| ATOM | 2395 | CD1 | ILE | 2051 | 30.167 | −17.024 | 71.044 | 1.00 | 36.90 |
| ATOM | 2396 | C | ILE | 2051 | 26.573 | −17.204 | 68.652 | 1.00 | 37.95 |
| ATOM | 2397 | O | ILE | 2051 | 26.620 | −16.026 | 68.299 | 1.00 | 37.78 |
| ATOM | 2398 | N | LYS | 2052 | 26.657 | −18.222 | 67.797 | 1.00 | 37.55 |
| ATOM | 2399 | CA | LYS | 2052 | 26.746 | −17.999 | 66.359 | 1.00 | 37.29 |
| ATOM | 2400 | CB | LYS | 2052 | 27.243 | −19.251 | 65.636 | 1.00 | 38.07 |
| ATOM | 2401 | CG | LYS | 2052 | 28.761 | −19.376 | 65.587 | 1.00 | 40.09 |
| ATOM | 2402 | CD | LYS | 2052 | 29.179 | −20.828 | 65.382 | 1.00 | 42.80 |
| ATOM | 2403 | CE | LYS | 2052 | 30.691 | −21.007 | 65.269 | 1.00 | 44.11 |
| ATOM | 2404 | NZ | LYS | 2052 | 31.219 | −20.552 | 63.952 | 1.00 | 45.23 |
| ATOM | 2405 | C | LYS | 2052 | 25.328 | −17.656 | 65.930 | 1.00 | 36.45 |
| ATOM | 2406 | O | LYS | 2052 | 24.420 | −18.483 | 66.020 | 1.00 | 36.36 |
| ATOM | 2407 | N | LEU | 2053 | 25.144 | −16.421 | 65.479 | 1.00 | 35.14 |
| ATOM | 2408 | CA | LEU | 2053 | 23.833 | −15.940 | 65.088 | 1.00 | 33.76 |
| ATOM | 2409 | CB | LEU | 2053 | 23.495 | −14.693 | 65.901 | 1.00 | 31.97 |
| ATOM | 2410 | CG | LEU | 2053 | 23.837 | −14.799 | 67.390 | 1.00 | 31.38 |
| ATOM | 2411 | CD1 | LEU | 2053 | 23.641 | −13.449 | 68.062 | 1.00 | 30.05 |
| ATOM | 2412 | CD2 | LEU | 2053 | 22.975 | −15.880 | 68.040 | 1.00 | 30.70 |
| ATOM | 2413 | C | LEU | 2053 | 23.741 | −15.622 | 63.613 | 1.00 | 33.64 |
| ATOM | 2414 | O | LEU | 2053 | 24.734 | −15.320 | 62.960 | 1.00 | 33.73 |
| ATOM | 2415 | N | GLN | 2054 | 22.530 | −15.686 | 63.087 | 1.00 | 33.50 |
| ATOM | 2416 | CA | GLN | 2054 | 22.319 | −15.398 | 61.685 | 1.00 | 33.49 |
| ATOM | 2417 | CB | GLN | 2054 | 21.703 | −16.629 | 61.000 | 1.00 | 33.45 |
| ATOM | 2418 | CG | GLN | 2054 | 21.718 | −16.587 | 59.490 | 1.00 | 34.53 |
| ATOM | 2419 | CD | GLN | 2054 | 23.117 | −16.480 | 58.920 | 1.00 | 35.58 |
| ATOM | 2420 | OE1 | GLN | 2054 | 23.928 | −17.394 | 59.052 | 1.00 | 36.54 |
| ATOM | 2421 | NE2 | GLN | 2054 | 23.407 | −15.356 | 58.283 | 1.00 | 36.90 |
| ATOM | 2422 | C | GLN | 2054 | 21.402 | −14.175 | 61.611 | 1.00 | 33.00 |
| ATOM | 2423 | O | GLN | 2054 | 20.210 | −14.252 | 61.907 | 1.00 | 32.54 |
| ATOM | 2424 | N | LEU | 2055 | 21.984 | −13.039 | 61.242 | 1.00 | 33.08 |
| ATOM | 2425 | CA | LEU | 2055 | 21.239 | −11.789 | 61.141 | 1.00 | 32.32 |
| ATOM | 2426 | CB | LEU | 2055 | 22.148 | −10.602 | 61.464 | 1.00 | 31.76 |
| ATOM | 2427 | CG | LEU | 2055 | 22.940 | −10.777 | 62.760 | 1.00 | 32.21 |
| ATOM | 2428 | CD1 | LEU | 2055 | 23.838 | −9.582 | 62.959 | 1.00 | 32.57 |
| ATOM | 2429 | CD2 | LEU | 2055 | 21.995 | −10.946 | 63.940 | 1.00 | 31.64 |
| ATOM | 2430 | C | LEU | 2055 | 20.724 | −11.695 | 59.720 | 1.00 | 32.13 |
| ATOM | 2431 | O | LEU | 2055 | 21.502 | −11.705 | 58.765 | 1.00 | 31.85 |
| ATOM | 2432 | N | GLN | 2056 | 19.406 | −11.606 | 59.596 | 1.00 | 31.81 |
| ATOM | 2433 | CA | GLN | 2056 | 18.757 | −11.558 | 58.304 | 1.00 | 32.00 |
| ATOM | 2434 | CB | GLN | 2056 | 17.941 | −12.840 | 58.123 | 1.00 | 31.28 |
| ATOM | 2435 | CG | GLN | 2056 | 17.171 | −12.936 | 56.827 | 1.00 | 33.33 |
| ATOM | 2436 | CD | GLN | 2056 | 18.081 | −12.922 | 55.610 | 1.00 | 34.94 |
| ATOM | 2437 | OE1 | GLN | 2056 | 18.966 | −13.782 | 55.467 | 1.00 | 35.08 |
| ATOM | 2438 | NE2 | GLN | 2056 | 17.870 | −11.945 | 54.720 | 1.00 | 34.69 |
| ATOM | 2439 | C | GLN | 2056 | 17.858 | −10.338 | 58.184 | 1.00 | 32.35 |
| ATOM | 2440 | O | GLN | 2056 | 16.957 | −10.142 | 58.996 | 1.00 | 33.08 |
| ATOM | 2441 | N | ALA | 2057 | 18.099 | −9.522 | 57.168 | 1.00 | 32.59 |
| ATOM | 2442 | CA | ALA | 2057 | 17.291 | −8.330 | 56.961 | 1.00 | 33.89 |
| ATOM | 2443 | CB | ALA | 2057 | 17.997 | −7.371 | 55.986 | 1.00 | 33.09 |
| ATOM | 2444 | C | ALA | 2057 | 15.928 | −8.742 | 56.408 | 1.00 | 34.76 |
| ATOM | 2445 | O | ALA | 2057 | 15.845 | −9.574 | 55.506 | 1.00 | 35.21 |
| ATOM | 2446 | N | GLU | 2058 | 14.863 | −8.173 | 56.963 | 1.00 | 35.09 |
| ATOM | 2447 | CA | GLU | 2058 | 13.509 | −8.470 | 56.500 | 1.00 | 36.09 |
| ATOM | 2448 | CB | GLU | 2058 | 12.534 | −8.482 | 57.677 | 1.00 | 37.01 |
| ATOM | 2449 | CG | GLU | 2058 | 11.245 | −9.210 | 57.406 | 1.00 | 38.98 |
| ATOM | 2450 | CD | GLU | 2058 | 11.470 | −10.585 | 56.780 | 1.00 | 40.69 |
| ATOM | 2451 | OE1 | GLU | 2058 | 12.426 | −11.285 | 57.185 | 1.00 | 40.31 |
| ATOM | 2452 | OE2 | GLU | 2058 | 10.680 | −10.965 | 55.884 | 1.00 | 42.10 |
| ATOM | 2453 | C | GLU | 2058 | 13.138 | −7.352 | 55.530 | 1.00 | 36.06 |
| ATOM | 2454 | O | GLU | 2058 | 12.346 | −7.529 | 54.610 | 1.00 | 35.63 |
| ATOM | 2455 | N | GLU | 2059 | 13.732 | −6.188 | 55.768 | 1.00 | 36.52 |
| ATOM | 2456 | CA | GLU | 2059 | 13.556 | −5.006 | 54.938 | 1.00 | 36.52 |
| ATOM | 2457 | CB | GLU | 2059 | 12.216 | −4.324 | 55.197 | 1.00 | 37.70 |
| ATOM | 2458 | CG | GLU | 2059 | 12.122 | −3.617 | 56.530 | 1.00 | 41.28 |
| ATOM | 2459 | CD | GLU | 2059 | 10.904 | −2.714 | 56.602 | 1.00 | 43.07 |
| ATOM | 2460 | OE1 | GLU | 2059 | 9.776 | −3.256 | 56.681 | 1.00 | 43.50 |
| ATOM | 2461 | OE2 | GLU | 2059 | 11.080 | −1.470 | 56.561 | 1.00 | 43.51 |
| ATOM | 2462 | C | GLU | 2059 | 14.695 | −4.076 | 55.326 | 1.00 | 35.67 |
| ATOM | 2463 | O | GLU | 2059 | 15.488 | −4.401 | 56.205 | 1.00 | 36.19 |
| ATOM | 2464 | N | ARG | 2060 | 14.774 | −2.918 | 54.696 | 1.00 | 34.63 |
| ATOM | 2465 | CA | ARG | 2060 | 15.847 | −1.993 | 54.993 | 1.00 | 34.30 |
| ATOM | 2466 | CB | ARG | 2060 | 15.691 | −0.733 | 54.135 | 1.00 | 36.44 |
| ATOM | 2467 | CG | ARG | 2060 | 17.008 | −0.253 | 53.532 | 1.00 | 40.62 |
| ATOM | 2468 | CD | ARG | 2060 | 16.826 | 0.420 | 52.173 | 1.00 | 43.95 |
| ATOM | 2469 | NE | ARG | 2060 | 16.131 | 1.705 | 52.264 | 1.00 | 47.78 |
| ATOM | 2470 | CZ | ARG | 2060 | 16.666 | 2.823 | 52.756 | 1.00 | 49.37 |
| ATOM | 2471 | NH1 | ARG | 2060 | 17.917 | 2.833 | 53.204 | 1.00 | 50.17 |
| ATOM | 2472 | NH2 | ARG | 2060 | 15.942 | 3.937 | 52.814 | 1.00 | 49.61 |
| ATOM | 2473 | C | ARG | 2060 | 15.977 | −1.631 | 56.477 | 1.00 | 33.34 |
| ATOM | 2474 | O | ARG | 2060 | 15.032 | −1.146 | 57.104 | 1.00 | 33.00 |
| ATOM | 2475 | N | GLY | 2061 | 17.164 | −1.894 | 57.031 | 1.00 | 32.18 |
| ATOM | 2476 | CA | GLY | 2061 | 17.454 | −1.584 | 58.420 | 1.00 | 30.12 |
| ATOM | 2477 | C | GLY | 2061 | 16.751 | −2.430 | 59.463 | 1.00 | 29.99 |
| ATOM | 2478 | O | GLY | 2061 | 16.916 | −2.187 | 60.663 | 1.00 | 29.62 |
| ATOM | 2479 | N | VAL | 2062 | 15.972 | −3.419 | 59.025 | 1.00 | 29.06 |
| ATOM | 2480 | CA | VAL | 2062 | 15.252 | −4.286 | 59.956 | 1.00 | 28.67 |
| ATOM | 2481 | CB | VAL | 2062 | 13.735 | −4.266 | 59.674 | 1.00 | 28.93 |
| ATOM | 2482 | CG1 | VAL | 2062 | 13.001 | −5.138 | 60.681 | 1.00 | 25.59 |
| ATOM | 2483 | CG2 | VAL | 2062 | 13.230 | −2.839 | 59.704 | 1.00 | 27.86 |
| ATOM | 2484 | C | VAL | 2062 | 15.735 | −5.721 | 59.838 | 1.00 | 28.68 |
| ATOM | 2485 | O | VAL | 2062 | 15.711 | −6.298 | 58.750 | 1.00 | 29.23 |
| ATOM | 2486 | N | VAL | 2063 | 16.163 | −6.305 | 60.951 | 1.00 | 27.83 |
| ATOM | 2487 | CA | VAL | 2063 | 16.645 | −7.679 | 60.916 | 1.00 | 28.10 |
| ATOM | 2488 | CB | VAL | 2063 | 18.179 | −7.773 | 61.154 | 1.00 | 28.42 |
| ATOM | 2489 | CG1 | VAL | 2063 | 18.944 | −6.994 | 60.082 | 1.00 | 27.05 |
| ATOM | 2490 | CG2 | VAL | 2063 | 18.514 | −7.300 | 62.577 | 1.00 | 27.83 |
| ATOM | 2491 | C | VAL | 2063 | 16.009 | −8.599 | 61.947 | 1.00 | 28.83 |
| ATOM | 2492 | O | VAL | 2063 | 15.340 | −8.152 | 62.886 | 1.00 | 29.34 |
| ATOM | 2493 | N | SER | 2064 | 16.223 | −9.897 | 61.750 | 1.00 | 28.76 |
| ATOM | 2494 | CA | SER | 2064 | 15.759 | −10.908 | 62.685 | 1.00 | 28.89 |
| ATOM | 2495 | CB | SER | 2064 | 14.844 | −11.930 | 62.009 | 1.00 | 28.14 |
| ATOM | 2496 | OG | SER | 2064 | 15.547 | −12.695 | 61.051 | 1.00 | 30.90 |
| ATOM | 2497 | C | SER | 2064 | 17.074 | −11.553 | 63.121 | 1.00 | 29.37 |
| ATOM | 2498 | O | SER | 2064 | 18.041 | −11.585 | 62.349 | 1.00 | 29.63 |
| ATOM | 2499 | N | ILE | 2065 | 17.126 | −12.032 | 64.356 | 1.00 | 29.64 |
| ATOM | 2500 | CA | ILE | 2065 | 18.339 | −12.635 | 64.877 | 1.00 | 30.62 |
| ATOM | 2501 | CB | ILE | 2065 | 18.823 | −11.852 | 66.100 | 1.00 | 30.96 |
| ATOM | 2502 | CG2 | ILE | 2065 | 20.064 | −12.500 | 66.687 | 1.00 | 30.71 |
| ATOM | 2503 | CG1 | ILE | 2065 | 19.090 | −10.403 | 65.688 | 1.00 | 31.15 |
| ATOM | 2504 | CD1 | ILE | 2065 | 19.278 | −9.463 | 66.858 | 1.00 | 32.40 |
| ATOM | 2505 | C | ILE | 2065 | 18.070 | −14.082 | 65.257 | 1.00 | 31.46 |
| ATOM | 2506 | O | ILE | 2065 | 17.350 | −14.364 | 66.220 | 1.00 | 31.52 |
| ATOM | 2507 | N | LYS | 2066 | 18.654 | −14.995 | 64.494 | 1.00 | 31.52 |
| ATOM | 2508 | CA | LYS | 2066 | 18.462 | −16.412 | 64.736 | 1.00 | 31.98 |
| ATOM | 2509 | CB | LYS | 2066 | 18.004 | −17.086 | 63.443 | 1.00 | 32.43 |
| ATOM | 2510 | CG | LYS | 2066 | 17.564 | −18.526 | 63.620 | 1.00 | 32.60 |
| ATOM | 2511 | CD | LYS | 2066 | 17.133 | −19.156 | 62.300 | 1.00 | 32.00 |
| ATOM | 2512 | CE | LYS | 2066 | 16.429 | −20.479 | 62.563 | 1.00 | 32.05 |
| ATOM | 2513 | NZ | LYS | 2066 | 16.029 | −21.165 | 61.318 | 1.00 | 31.68 |
| ATOM | 2514 | C | LYS | 2066 | 19.699 | −17.126 | 65.277 | 1.00 | 32.24 |
| ATOM | 2515 | O | LYS | 2066 | 20.801 | −17.015 | 64.725 | 1.00 | 31.06 |
| ATOM | 2516 | N | GLY | 2067 | 19.506 | −17.854 | 66.373 | 1.00 | 33.23 |
| ATOM | 2517 | CA | GLY | 2067 | 20.596 | −18.614 | 66.961 | 1.00 | 34.02 |
| ATOM | 2518 | C | GLY | 2067 | 20.709 | −19.868 | 66.123 | 1.00 | 34.15 |
| ATOM | 2519 | O | GLY | 2067 | 19.818 | −20.703 | 66.143 | 1.00 | 34.33 |
| ATOM | 2520 | N | VAL | 2068 | 21.795 | −20.004 | 65.381 | 1.00 | 34.47 |
| ATOM | 2521 | CA | VAL | 2068 | 21.964 | −21.152 | 64.509 | 1.00 | 35.26 |
| ATOM | 2522 | CB | VAL | 2068 | 23.340 | −21.172 | 63.864 | 1.00 | 34.03 |
| ATOM | 2523 | CG1 | VAL | 2068 | 23.397 | −22.292 | 62.842 | 1.00 | 33.08 |
| ATOM | 2524 | CG2 | VAL | 2068 | 23.629 | −19.833 | 63.217 | 1.00 | 33.92 |
| ATOM | 2525 | C | VAL | 2068 | 21.731 | −22.518 | 65.132 | 1.00 | 36.64 |
| ATOM | 2526 | O | VAL | 2068 | 20.877 | −23.272 | 64.664 | 1.00 | 36.95 |
| ATOM | 2527 | N | SER | 2069 | 22.479 | −22.854 | 66.175 | 1.00 | 37.60 |
| ATOM | 2528 | CA | SER | 2069 | 22.305 | −24.163 | 66.778 | 1.00 | 38.37 |
| ATOM | 2529 | CB | SER | 2069 | 23.465 | −24.475 | 67.718 | 1.00 | 37.74 |
| ATOM | 2530 | OG | SER | 2069 | 23.287 | −23.849 | 68.969 | 1.00 | 38.57 |
| ATOM | 2531 | C | SER | 2069 | 20.972 | −24.290 | 67.518 | 1.00 | 38.94 |
| ATOM | 2532 | O | SER | 2069 | 20.374 | −25.361 | 67.548 | 1.00 | 39.56 |
| ATOM | 2533 | N | ALA | 2070 | 20.493 | −23.205 | 68.108 | 1.00 | 39.13 |
| ATOM | 2534 | CA | ALA | 2070 | 19.226 | −23.266 | 68.827 | 1.00 | 39.53 |
| ATOM | 2535 | CB | ALA | 2070 | 19.092 | −22.052 | 69.733 | 1.00 | 39.50 |
| ATOM | 2536 | C | ALA | 2070 | 18.036 | −23.324 | 67.870 | 1.00 | 39.62 |
| ATOM | 2537 | O | ALA | 2070 | 16.938 | −23.730 | 68.251 | 1.00 | 39.72 |
| ATOM | 2538 | N | ASN | 2071 | 18.267 | −22.909 | 66.631 | 1.00 | 39.29 |
| ATOM | 2539 | CA | ASN | 2071 | 17.227 | −22.865 | 65.618 | 1.00 | 39.24 |
| ATOM | 2540 | CB | ASN | 2071 | 16.799 | −24.281 | 65.220 | 1.00 | 38.60 |
| ATOM | 2541 | CG | ASN | 2071 | 15.864 | −24.301 | 64.005 | 1.00 | 37.85 |
| ATOM | 2542 | OD1 | ASN | 2071 | 15.928 | −23.430 | 63.138 | 1.00 | 36.58 |
| ATOM | 2543 | ND2 | ASN | 2071 | 15.008 | −25.316 | 63.934 | 1.00 | 37.52 |
| ATOM | 2544 | C | ASN | 2071 | 16.023 | −22.058 | 66.113 | 1.00 | 39.52 |
| ATOM | 2545 | O | ASN | 2071 | 14.873 | −22.402 | 65.842 | 1.00 | 39.65 |
| ATOM | 2546 | N | ARG | 2072 | 16.297 | −20.984 | 66.851 | 1.00 | 39.82 |
| ATOM | 2547 | CA | ARG | 2072 | 15.245 | −20.108 | 67.357 | 1.00 | 39.51 |
| ATOM | 2548 | CB | ARG | 2072 | 15.056 | −20.305 | 68.853 | 1.00 | 40.54 |
| ATOM | 2549 | CG | ARG | 2072 | 14.547 | −21.676 | 69.209 | 1.00 | 43.60 |
| ATOM | 2550 | CD | ARG | 2072 | 13.878 | −21.615 | 70.552 | 1.00 | 46.83 |
| ATOM | 2551 | NE | ARG | 2072 | 13.382 | −22.902 | 71.032 | 1.00 | 49.54 |
| ATOM | 2552 | CZ | ARG | 2072 | 12.627 | −23.033 | 72.122 | 1.00 | 50.83 |
| ATOM | 2553 | NH1 | ARG | 2072 | 12.287 | −21.958 | 72.827 | 1.00 | 50.54 |
| ATOM | 2554 | NH2 | ARG | 2072 | 12.212 | −24.233 | 72.515 | 1.00 | 51.79 |
| ATOM | 2555 | C | ARG | 2072 | 15.565 | −18.646 | 67.060 | 1.00 | 38.30 |
| ATOM | 2556 | O | ARG | 2072 | 16.723 | −18.278 | 66.904 | 1.00 | 38.11 |
| ATOM | 2557 | N | TYR | 2073 | 14.528 | −17.823 | 66.974 | 1.00 | 37.82 |
| ATOM | 2558 | CA | TYR | 2073 | 14.678 | −16.401 | 66.693 | 1.00 | 36.97 |
| ATOM | 2559 | CB | TYR | 2073 | 13.618 | −15.916 | 65.697 | 1.00 | 36.26 |
| ATOM | 2560 | CG | TYR | 2073 | 13.572 | −16.671 | 64.386 | 1.00 | 35.32 |
| ATOM | 2561 | CD1 | TYR | 2073 | 12.781 | −17.813 | 64.243 | 1.00 | 34.14 |
| ATOM | 2562 | CE1 | TYR | 2073 | 12.755 | −18.515 | 63.057 | 1.00 | 34.44 |
| ATOM | 2563 | CD2 | TYR | 2073 | 14.335 | −16.254 | 63.298 | 1.00 | 34.61 |
| ATOM | 2564 | CE2 | TYR | 2073 | 14.317 | −16.951 | 62.103 | 1.00 | 34.47 |
| ATOM | 2565 | CZ | TYR | 2073 | 13.525 | −18.080 | 61.987 | 1.00 | 34.84 |
| ATOM | 2566 | OH | TYR | 2073 | 13.498 | −18.763 | 60.799 | 1.00 | 34.76 |
| ATOM | 2567 | C | TYR | 2073 | 14.533 | −15.572 | 67.952 | 1.00 | 37.28 |
| ATOM | 2568 | O | TYR | 2073 | 13.648 | −15.818 | 68.773 | 1.00 | 38.26 |
| ATOM | 2569 | N | LEU | 2074 | 15.393 | −14.572 | 68.099 | 1.00 | 37.10 |
| ATOM | 2570 | CA | LEU | 2074 | 15.316 | −13.707 | 69.264 | 1.00 | 37.13 |
| ATOM | 2571 | CB | LEU | 2074 | 16.524 | −12.771 | 69.331 | 1.00 | 37.25 |
| ATOM | 2572 | CG | LEU | 2074 | 16.515 | −11.847 | 70.551 | 1.00 | 36.81 |
| ATOM | 2573 | CD1 | LEU | 2074 | 16.651 | −12.676 | 71.810 | 1.00 | 37.56 |
| ATOM | 2574 | CD2 | LEU | 2074 | 17.647 | −10.855 | 70.464 | 1.00 | 37.34 |
| ATOM | 2575 | C | LEU | 2074 | 14.043 | −12.877 | 69.186 | 1.00 | 36.77 |
| ATOM | 2576 | O | LEU | 2074 | 13.675 | −12.389 | 68.121 | 1.00 | 36.01 |
| ATOM | 2577 | N | ALA | 2075 | 13.371 | −12.732 | 70.322 | 1.00 | 37.58 |
| ATOM | 2578 | CA | ALA | 2075 | 12.142 | −11.954 | 70.404 | 1.00 | 37.98 |
| ATOM | 2579 | CB | ALA | 2075 | 10.930 | −12.858 | 70.267 | 1.00 | 37.74 |
| ATOM | 2580 | C | ALA | 2075 | 12.091 | −11.229 | 71.733 | 1.00 | 38.71 |
| ATOM | 2581 | O | ALA | 2075 | 12.571 | −11.732 | 72.745 | 1.00 | 39.10 |
| ATOM | 2582 | N | MET | 2076 | 11.512 | −10.038 | 71.712 | 1.00 | 40.02 |
| ATOM | 2583 | CA | MET | 2076 | 11.371 | −9.208 | 72.896 | 1.00 | 41.74 |
| ATOM | 2584 | CB | MET | 2076 | 11.921 | −7.818 | 72.615 | 1.00 | 41.95 |
| ATOM | 2585 | CG | MET | 2076 | 11.277 | −6.745 | 73.432 | 1.00 | 41.51 |
| ATOM | 2586 | SD | MET | 2076 | 12.538 | −5.778 | 74.186 | 1.00 | 44.27 |
| ATOM | 2587 | CE | MET | 2076 | 12.886 | −4.666 | 72.901 | 1.00 | 42.71 |
| ATOM | 2588 | C | MET | 2076 | 9.890 | −9.110 | 73.206 | 1.00 | 43.03 |
| ATOM | 2589 | O | MET | 2076 | 9.097 | −8.779 | 72.326 | 1.00 | 43.27 |
| ATOM | 2590 | N | LYS | 2077 | 9.514 | −9.373 | 74.455 | 1.00 | 44.30 |
| ATOM | 2591 | CA | LYS | 2077 | 8.106 | −9.350 | 74.834 | 1.00 | 45.41 |
| ATOM | 2592 | CB | LYS | 2077 | 7.871 | −10.352 | 75.957 | 1.00 | 46.11 |
| ATOM | 2593 | CG | LYS | 2077 | 8.430 | −11.744 | 75.667 | 1.00 | 46.92 |
| ATOM | 2594 | CD | LYS | 2077 | 7.394 | −12.661 | 75.048 | 1.00 | 48.11 |
| ATOM | 2595 | CE | LYS | 2077 | 6.890 | −12.138 | 73.728 | 1.00 | 48.17 |
| ATOM | 2596 | NZ | LYS | 2077 | 5.851 | −13.050 | 73.180 | 1.00 | 48.97 |
| ATOM | 2597 | C | LYS | 2077 | 7.600 | −7.978 | 75.250 | 1.00 | 46.51 |
| ATOM | 2598 | O | LYS | 2077 | 8.370 | −7.023 | 75.343 | 1.00 | 46.90 |
| ATOM | 2599 | N | GLU | 2078 | 6.294 | −7.895 | 75.501 | 1.00 | 47.42 |
| ATOM | 2600 | CA | GLU | 2078 | 5.646 | −6.645 | 75.900 | 1.00 | 48.45 |
| ATOM | 2601 | CB | GLU | 2078 | 4.121 | −6.834 | 75.989 | 1.00 | 50.69 |
| ATOM | 2602 | CG | GLU | 2078 | 3.315 | −5.623 | 76.542 | 1.00 | 53.33 |
| ATOM | 2603 | CD | GLU | 2078 | 3.034 | −4.520 | 75.506 | 1.00 | 55.23 |
| ATOM | 2604 | OE1 | GLU | 2078 | 2.585 | −4.842 | 74.379 | 1.00 | 56.62 |
| ATOM | 2605 | OE2 | GLU | 2078 | 3.239 | −3.326 | 75.826 | 1.00 | 55.05 |
| ATOM | 2606 | C | GLU | 2078 | 6.171 | −6.097 | 77.221 | 1.00 | 48.14 |
| ATOM | 2607 | O | GLU | 2078 | 6.101 | −4.893 | 77.460 | 1.00 | 48.16 |
| ATOM | 2608 | N | ASP | 2079 | 6.690 | −6.970 | 78.079 | 1.00 | 47.57 |
| ATOM | 2609 | CA | ASP | 2079 | 7.224 | −6.535 | 79.367 | 1.00 | 47.37 |
| ATOM | 2610 | CB | ASP | 2079 | 7.067 | −7.645 | 80.402 | 1.00 | 48.16 |
| ATOM | 2611 | CG | ASP | 2079 | 7.695 | −8.948 | 79.949 | 1.00 | 49.56 |
| ATOM | 2612 | OD1 | ASP | 2079 | 7.915 | −9.836 | 80.806 | 1.00 | 50.08 |
| ATOM | 2613 | OD2 | ASP | 2079 | 7.965 | −9.081 | 78.732 | 1.00 | 50.00 |
| ATOM | 2614 | C | ASP | 2079 | 8.702 | −6.162 | 79.240 | 1.00 | 46.87 |
| ATOM | 2615 | O | ASP | 2079 | 9.283 | −5.568 | 80.152 | 1.00 | 47.20 |
| ATOM | 2616 | N | GLY | 2080 | 9.305 | −6.526 | 78.110 | 1.00 | 45.91 |
| ATOM | 2617 | CA | GLY | 2080 | 10.699 | −6.204 | 77.879 | 1.00 | 44.80 |
| ATOM | 2618 | C | GLY | 2080 | 11.651 | −7.369 | 78.017 | 1.00 | 44.39 |
| ATOM | 2619 | O | GLY | 2080 | 12.845 | −7.222 | 77.767 | 1.00 | 44.73 |
| ATOM | 2620 | N | ARG | 2081 | 11.140 | −8.530 | 78.409 | 1.00 | 43.97 |
| ATOM | 2621 | CA | ARG | 2081 | 11.991 | −9.702 | 78.574 | 1.00 | 43.45 |
| ATOM | 2622 | CB | ARG | 2081 | 11.286 | −10.739 | 79.457 | 1.00 | 45.01 |
| ATOM | 2623 | CG | ARG | 2081 | 9.959 | −11.278 | 78.941 | 1.00 | 46.70 |
| ATOM | 2624 | CD | ARG | 2081 | 9.276 | −12.117 | 80.047 | 1.00 | 48.80 |
| ATOM | 2625 | NE | ARG | 2081 | 8.344 | −13.134 | 79.543 | 1.00 | 50.51 |
| ATOM | 2626 | CZ | ARG | 2081 | 7.191 | −12.884 | 78.919 | 1.00 | 51.28 |
| ATOM | 2627 | NH1 | ARG | 2081 | 6.789 | −11.636 | 78.703 | 1.00 | 51.35 |
| ATOM | 2628 | NH2 | ARG | 2081 | 6.436 | −13.894 | 78.502 | 1.00 | 51.55 |
| ATOM | 2629 | C | ARG | 2081 | 12.373 | −10.301 | 77.225 | 1.00 | 42.17 |
| ATOM | 2630 | O | ARG | 2081 | 11.666 | −10.115 | 76.238 | 1.00 | 41.65 |
| ATOM | 2631 | N | LEU | 2082 | 13.501 | −11.000 | 77.180 | 1.00 | 40.89 |
| ATOM | 2632 | CA | LEU | 2082 | 13.969 | −11.605 | 75.935 | 1.00 | 40.35 |
| ATOM | 2633 | CB | LEU | 2082 | 15.432 | −11.236 | 75.659 | 1.00 | 39.12 |
| ATOM | 2634 | CG | LEU | 2082 | 15.849 | −9.788 | 75.415 | 1.00 | 37.50 |
| ATOM | 2635 | CD1 | LEU | 2082 | 17.345 | −9.745 | 75.136 | 1.00 | 36.70 |
| ATOM | 2636 | CD2 | LEU | 2082 | 15.081 | −9.224 | 74.241 | 1.00 | 37.90 |
| ATOM | 2637 | C | LEU | 2082 | 13.869 | −13.121 | 75.957 | 1.00 | 40.65 |
| ATOM | 2638 | O | LEU | 2082 | 14.028 | −13.755 | 76.997 | 1.00 | 41.43 |
| ATOM | 2639 | N | LEU | 2083 | 13.613 | −13.704 | 74.798 | 1.00 | 40.76 |
| ATOM | 2640 | CA | LEU | 2083 | 13.523 | −15.148 | 74.704 | 1.00 | 40.82 |
| ATOM | 2641 | CB | LEU | 2083 | 12.181 | −15.636 | 75.266 | 1.00 | 40.49 |
| ATOM | 2642 | CG | LEU | 2083 | 10.878 | −15.343 | 74.527 | 1.00 | 40.14 |
| ATOM | 2643 | CD1 | LEU | 2083 | 10.746 | −16.282 | 73.343 | 1.00 | 40.58 |
| ATOM | 2644 | CD2 | LEU | 2083 | 9.698 | −15.539 | 75.469 | 1.00 | 39.37 |
| ATOM | 2645 | C | LEU | 2083 | 13.684 | −15.536 | 73.250 | 1.00 | 40.87 |
| ATOM | 2646 | O | LEU | 2083 | 13.589 | −14.691 | 72.365 | 1.00 | 41.06 |
| ATOM | 2647 | N | ALA | 2084 | 13.928 | −16.815 | 73.007 | 1.00 | 41.02 |
| ATOM | 2648 | CA | ALA | 2084 | 14.116 | −17.300 | 71.652 | 1.00 | 41.88 |
| ATOM | 2649 | CB | ALA | 2084 | 15.367 | −18.157 | 71.596 | 1.00 | 42.75 |
| ATOM | 2650 | C | ALA | 2084 | 12.909 | −18.111 | 71.185 | 1.00 | 41.93 |
| ATOM | 2651 | O | ALA | 2084 | 12.722 | −19.246 | 71.607 | 1.00 | 42.13 |
| ATOM | 2652 | N | SER | 2085 | 12.103 | −17.518 | 70.312 | 1.00 | 41.29 |
| ATOM | 2653 | CA | SER | 2085 | 10.921 | −18.167 | 69.772 | 1.00 | 40.96 |
| ATOM | 2654 | CB | SER | 2085 | 9.960 | −17.100 | 69.253 | 1.00 | 40.52 |
| ATOM | 2655 | OG | SER | 2085 | 9.014 | −17.661 | 68.373 | 1.00 | 39.48 |
| ATOM | 2656 | C | SER | 2085 | 11.265 | −19.143 | 68.643 | 1.00 | 41.48 |
| ATOM | 2657 | O | SER | 2085 | 12.199 | −18.917 | 67.878 | 1.00 | 40.96 |
| ATOM | 2658 | N | LYS | 2086 | 10.500 | −20.227 | 68.541 | 1.00 | 41.91 |
| ATOM | 2659 | CA | LYS | 2086 | 10.719 | −21.231 | 67.508 | 1.00 | 42.54 |
| ATOM | 2660 | CB | LYS | 2086 | 9.999 | −22.530 | 67.874 | 1.00 | 43.38 |
| ATOM | 2661 | CG | LYS | 2086 | 10.840 | −23.776 | 67.675 | 1.00 | 44.61 |
| ATOM | 2662 | CD | LYS | 2086 | 12.044 | −23.777 | 68.637 | 1.00 | 46.00 |
| ATOM | 2663 | CE | LYS | 2086 | 12.963 | −24.990 | 68.434 | 1.00 | 46.34 |
| ATOM | 2664 | NZ | LYS | 2086 | 13.473 | −25.081 | 67.027 | 1.00 | 44.33 |
| ATOM | 2665 | C | LYS | 2086 | 10.203 | −20.728 | 66.165 | 1.00 | 42.50 |
| ATOM | 2666 | O | LYS | 2086 | 10.731 | −21.081 | 65.110 | 1.00 | 42.30 |
| ATOM | 2667 | N | SER | 2087 | 9.163 | −19.904 | 66.205 | 1.00 | 42.70 |
| ATOM | 2668 | CA | SER | 2087 | 8.591 | −19.350 | 64.979 | 1.00 | 43.10 |
| ATOM | 2669 | CB | SER | 2087 | 7.101 | −19.685 | 64.867 | 1.00 | 43.18 |
| ATOM | 2670 | OG | SER | 2087 | 6.354 | −18.964 | 65.831 | 1.00 | 44.33 |
| ATOM | 2671 | C | SER | 2087 | 8.752 | −17.835 | 64.940 | 1.00 | 42.87 |
| ATOM | 2672 | O | SER | 2087 | 8.925 | −17.189 | 65.969 | 1.00 | 42.02 |
| ATOM | 2673 | N | VAL | 2088 | 8.667 | −17.280 | 63.739 | 1.00 | 43.04 |
| ATOM | 2674 | CA | VAL | 2088 | 8.811 | −15.846 | 63.523 | 1.00 | 43.55 |
| ATOM | 2675 | CB | VAL | 2088 | 9.205 | −15.568 | 62.055 | 1.00 | 43.95 |
| ATOM | 2676 | CG1 | VAL | 2088 | 9.347 | −14.072 | 61.823 | 1.00 | 44.00 |
| ATOM | 2677 | CG2 | VAL | 2088 | 10.492 | −16.301 | 61.707 | 1.00 | 43.77 |
| ATOM | 2678 | C | VAL | 2088 | 7.535 | −15.066 | 63.801 | 1.00 | 43.80 |
| ATOM | 2679 | O | VAL | 2088 | 6.520 | −15.314 | 63.169 | 1.00 | 44.15 |
| ATOM | 2680 | N | THR | 2089 | 7.596 | −14.112 | 64.726 | 1.00 | 44.65 |
| ATOM | 2681 | CA | THR | 2089 | 6.439 | −13.276 | 65.062 | 1.00 | 44.85 |
| ATOM | 2682 | CB | THR | 2089 | 5.991 | −13.509 | 66.518 | 1.00 | 45.06 |
| ATOM | 2683 | OG1 | THR | 2089 | 6.766 | −12.682 | 67.398 | 1.00 | 44.77 |
| ATOM | 2684 | CG2 | THR | 2089 | 6.200 | −14.969 | 66.908 | 1.00 | 44.86 |
| ATOM | 2685 | C | THR | 2089 | 6.883 | −11.818 | 64.911 | 1.00 | 45.09 |
| ATOM | 2686 | O | THR | 2089 | 8.071 | −11.554 | 64.729 | 1.00 | 45.51 |
| ATOM | 2687 | N | ASP | 2090 | 5.949 | −10.874 | 64.992 | 1.00 | 44.82 |
| ATOM | 2688 | CA | ASP | 2090 | 6.313 | −9.466 | 64.851 | 1.00 | 44.41 |
| ATOM | 2689 | CB | ASP | 2090 | 5.063 | −8.569 | 64.780 | 1.00 | 45.63 |
| ATOM | 2690 | CG | ASP | 2090 | 4.188 | −8.668 | 66.024 | 1.00 | 47.61 |
| ATOM | 2691 | OD1 | ASP | 2090 | 4.732 | −8.901 | 67.123 | 1.00 | 49.03 |
| ATOM | 2692 | OD2 | ASP | 2090 | 2.952 | −8.498 | 65.909 | 1.00 | 49.18 |
| ATOM | 2693 | C | ASP | 2090 | 7.233 | −8.983 | 65.973 | 1.00 | 43.36 |
| ATOM | 2694 | O | ASP | 2090 | 7.758 | −7.873 | 65.913 | 1.00 | 44.14 |
| ATOM | 2695 | N | GLU | 2091 | 7.436 | −9.813 | 66.989 | 1.00 | 41.65 |
| ATOM | 2696 | CA | GLU | 2091 | 8.300 | −9.447 | 68.109 | 1.00 | 40.75 |
| ATOM | 2697 | CB | GLU | 2091 | 7.784 | −10.089 | 69.402 | 1.00 | 40.50 |
| ATOM | 2698 | CG | GLU | 2091 | 6.350 | −9.721 | 69.751 | 1.00 | 40.99 |
| ATOM | 2699 | CD | GLU | 2091 | 5.874 | −10.386 | 71.032 | 1.00 | 41.13 |
| ATOM | 2700 | OE1 | GLU | 2091 | 5.976 | −11.627 | 71.130 | 1.00 | 41.67 |
| ATOM | 2701 | OE2 | GLU | 2091 | 5.396 | −9.668 | 71.937 | 1.00 | 40.91 |
| ATOM | 2702 | C | GLU | 2091 | 9.739 | −9.907 | 67.872 | 1.00 | 39.84 |
| ATOM | 2703 | O | GLU | 2091 | 10.591 | −9.813 | 68.763 | 1.00 | 39.50 |
| ATOM | 2704 | N | CYS | 2092 | 10.002 | −10.403 | 66.669 | 1.00 | 38.39 |
| ATOM | 2705 | CA | CYS | 2092 | 11.318 | −10.908 | 66.331 | 1.00 | 38.25 |
| ATOM | 2706 | CB | CYS | 2092 | 11.186 | −12.286 | 65.667 | 1.00 | 38.84 |
| ATOM | 2707 | SG | CYS | 2092 | 10.481 | −13.586 | 66.728 | 1.00 | 39.99 |
| ATOM | 2708 | C | CYS | 2092 | 12.119 | −9.982 | 65.425 | 1.00 | 37.45 |
| ATOM | 2709 | O | CYS | 2092 | 13.199 | −10.349 | 64.963 | 1.00 | 38.08 |
| ATOM | 2710 | N | PHE | 2093 | 11.598 | −8.786 | 65.182 | 1.00 | 36.01 |
| ATOM | 2711 | CA | PHE | 2093 | 12.273 | −7.838 | 64.312 | 1.00 | 35.06 |
| ATOM | 2712 | CB | PHE | 2093 | 11.320 | −7.443 | 63.194 | 1.00 | 34.60 |
| ATOM | 2713 | CG | PHE | 2093 | 10.966 | −8.597 | 62.308 | 1.00 | 35.16 |
| ATOM | 2714 | CD1 | PHE | 2093 | 11.946 | −9.209 | 61.529 | 1.00 | 34.45 |
| ATOM | 2715 | CD2 | PHE | 2093 | 9.671 | −9.112 | 62.290 | 1.00 | 35.37 |
| ATOM | 2716 | CE1 | PHE | 2093 | 11.646 | −10.312 | 60.752 | 1.00 | 34.78 |
| ATOM | 2717 | CE2 | PHE | 2093 | 9.360 | −10.215 | 61.515 | 1.00 | 34.16 |
| ATOM | 2718 | CZ | PHE | 2093 | 10.349 | −10.818 | 60.743 | 1.00 | 34.73 |
| ATOM | 2719 | C | PHE | 2093 | 12.840 | −6.623 | 65.032 | 1.00 | 34.48 |
| ATOM | 2720 | O | PHE | 2093 | 12.202 | −6.043 | 65.912 | 1.00 | 34.85 |
| ATOM | 2721 | N | PHE | 2094 | 14.055 | −6.251 | 64.650 | 1.00 | 33.10 |
| ATOM | 2722 | CA | PHE | 2094 | 14.749 | −5.143 | 65.284 | 1.00 | 32.10 |
| ATOM | 2723 | CB | PHE | 2094 | 15.850 | −5.705 | 66.188 | 1.00 | 31.86 |
| ATOM | 2724 | CG | PHE | 2094 | 15.357 | −6.714 | 67.187 | 1.00 | 31.67 |
| ATOM | 2725 | CD1 | PHE | 2094 | 14.917 | −6.311 | 68.454 | 1.00 | 31.90 |
| ATOM | 2726 | CD2 | PHE | 2094 | 15.270 | −8.062 | 66.841 | 1.00 | 31.77 |
| ATOM | 2727 | CE1 | PHE | 2094 | 14.392 | −7.232 | 69.359 | 1.00 | 31.21 |
| ATOM | 2728 | CE2 | PHE | 2094 | 14.743 | −9.003 | 67.737 | 1.00 | 31.89 |
| ATOM | 2729 | CZ | PHE | 2094 | 14.303 | −8.585 | 68.998 | 1.00 | 32.44 |
| ATOM | 2730 | C | PHE | 2094 | 15.381 | −4.194 | 64.278 | 1.00 | 31.49 |
| ATOM | 2731 | O | PHE | 2094 | 15.798 | −4.609 | 63.201 | 1.00 | 31.85 |
| ATOM | 2732 | N | PHE | 2095 | 15.441 | −2.915 | 64.631 | 1.00 | 30.41 |
| ATOM | 2733 | CA | PHE | 2095 | 16.082 | −1.940 | 63.771 | 1.00 | 29.72 |
| ATOM | 2734 | CB | PHE | 2095 | 15.601 | −0.536 | 64.098 | 1.00 | 29.28 |
| ATOM | 2735 | CG | PHE | 2095 | 14.183 | −0.281 | 63.697 | 1.00 | 28.68 |
| ATOM | 2736 | CD1 | PHE | 2095 | 13.220 | 0.026 | 64.652 | 1.00 | 27.83 |
| ATOM | 2737 | CD2 | PHE | 2095 | 13.801 | −0.370 | 62.361 | 1.00 | 28.43 |
| ATOM | 2738 | CE1 | PHE | 2095 | 11.907 | 0.236 | 64.287 | 1.00 | 27.10 |
| ATOM | 2739 | CE2 | PHE | 2095 | 12.479 | −0.158 | 61.988 | 1.00 | 27.81 |
| ATOM | 2740 | CZ | PHE | 2095 | 11.535 | 0.145 | 62.957 | 1.00 | 27.56 |
| ATOM | 2741 | C | PHE | 2095 | 17.554 | −2.052 | 64.087 | 1.00 | 29.79 |
| ATOM | 2742 | O | PHE | 2095 | 17.962 | −1.835 | 65.226 | 1.00 | 30.24 |
| ATOM | 2743 | N | GLU | 2096 | 18.349 | −2.429 | 63.096 | 1.00 | 29.84 |
| ATOM | 2744 | CA | GLU | 2096 | 19.784 | −2.548 | 63.306 | 1.00 | 29.84 |
| ATOM | 2745 | CB | GLU | 2096 | 20.384 | −3.694 | 62.492 | 1.00 | 29.37 |
| ATOM | 2746 | CG | GLU | 2096 | 21.895 | −3.816 | 62.677 | 1.00 | 28.81 |
| ATOM | 2747 | CD | GLU | 2096 | 22.530 | −4.840 | 61.750 | 1.00 | 29.13 |
| ATOM | 2748 | OE1 | GLU | 2096 | 22.339 | −4.732 | 60.518 | 1.00 | 29.29 |
| ATOM | 2749 | OE2 | GLU | 2096 | 23.230 | −5.746 | 62.252 | 1.00 | 28.34 |
| ATOM | 2750 | C | GLU | 2096 | 20.431 | −1.244 | 62.878 | 1.00 | 30.21 |
| ATOM | 2751 | O | GLU | 2096 | 20.327 | −0.824 | 61.720 | 1.00 | 30.33 |
| ATOM | 2752 | N | ARG | 2097 | 21.109 | −0.602 | 63.813 | 1.00 | 30.18 |
| ATOM | 2753 | CA | ARG | 2097 | 21.751 | 0.655 | 63.509 | 1.00 | 29.84 |
| ATOM | 2754 | CB | ARG | 2097 | 21.036 | 1.770 | 64.270 | 1.00 | 30.45 |
| ATOM | 2755 | CG | ARG | 2097 | 21.752 | 3.094 | 64.295 | 1.00 | 33.23 |
| ATOM | 2756 | CD | ARG | 2097 | 20.823 | 4.205 | 64.789 | 1.00 | 34.88 |
| ATOM | 2757 | NE | ARG | 2097 | 21.532 | 5.474 | 64.948 | 1.00 | 38.02 |
| ATOM | 2758 | CZ | ARG | 2097 | 22.111 | 6.155 | 63.955 | 1.00 | 39.80 |
| ATOM | 2759 | NH1 | ARG | 2097 | 22.067 | 5.698 | 62.702 | 1.00 | 39.80 |
| ATOM | 2760 | NH2 | ARG | 2097 | 22.763 | 7.285 | 64.221 | 1.00 | 39.52 |
| ATOM | 2761 | C | ARG | 2097 | 23.229 | 0.658 | 63.849 | 1.00 | 29.17 |
| ATOM | 2762 | O | ARG | 2097 | 23.632 | 0.228 | 64.929 | 1.00 | 29.13 |
| ATOM | 2763 | N | LEU | 2098 | 24.034 | 1.124 | 62.900 | 1.00 | 28.72 |
| ATOM | 2764 | CA | LEU | 2098 | 25.478 | 1.256 | 63.095 | 1.00 | 28.55 |
| ATOM | 2765 | CB | LEU | 2098 | 26.216 | 1.141 | 61.749 | 1.00 | 28.60 |
| ATOM | 2766 | CG | LEU | 2098 | 27.674 | 1.623 | 61.601 | 1.00 | 28.87 |
| ATOM | 2767 | CD1 | LEU | 2098 | 28.535 | 1.208 | 62.785 | 1.00 | 29.74 |
| ATOM | 2768 | CD2 | LEU | 2098 | 28.243 | 1.035 | 60.321 | 1.00 | 28.98 |
| ATOM | 2769 | C | LEU | 2098 | 25.618 | 2.666 | 63.671 | 1.00 | 28.37 |
| ATOM | 2770 | O | LEU | 2098 | 25.579 | 3.653 | 62.939 | 1.00 | 28.15 |
| ATOM | 2771 | N | GLU | 2099 | 25.740 | 2.755 | 64.990 | 1.00 | 27.71 |
| ATOM | 2772 | CA | GLU | 2099 | 25.850 | 4.049 | 65.650 | 1.00 | 27.87 |
| ATOM | 2773 | CB | GLU | 2099 | 25.787 | 3.883 | 67.171 | 1.00 | 27.75 |
| ATOM | 2774 | CG | GLU | 2099 | 24.598 | 3.084 | 67.676 | 1.00 | 30.30 |
| ATOM | 2775 | CD | GLU | 2099 | 23.246 | 3.800 | 67.548 | 1.00 | 31.10 |
| ATOM | 2776 | OE1 | GLU | 2099 | 22.220 | 3.138 | 67.805 | 1.00 | 32.73 |
| ATOM | 2777 | OE2 | GLU | 2099 | 23.190 | 5.007 | 67.209 | 1.00 | 31.03 |
| ATOM | 2778 | C | GLU | 2099 | 27.142 | 4.787 | 65.292 | 1.00 | 28.33 |
| ATOM | 2779 | O | GLU | 2099 | 28.117 | 4.195 | 64.807 | 1.00 | 27.42 |
| ATOM | 2780 | N | SER | 2100 | 27.144 | 6.087 | 65.568 | 1.00 | 28.08 |
| ATOM | 2781 | CA | SER | 2100 | 28.296 | 6.924 | 65.285 | 1.00 | 27.90 |
| ATOM | 2782 | CB | SER | 2100 | 27.959 | 8.400 | 65.537 | 1.00 | 27.71 |
| ATOM | 2783 | OG | SER | 2100 | 27.495 | 8.619 | 66.864 | 1.00 | 31.20 |
| ATOM | 2784 | C | SER | 2100 | 29.519 | 6.513 | 66.096 | 1.00 | 27.60 |
| ATOM | 2785 | O | SER | 2100 | 30.638 | 6.864 | 65.748 | 1.00 | 27.91 |
| ATOM | 2786 | N | ASN | 2101 | 29.323 | 5.769 | 67.174 | 1.00 | 27.41 |
| ATOM | 2787 | CA | ASN | 2101 | 30.471 | 5.342 | 67.964 | 1.00 | 27.39 |
| ATOM | 2788 | CB | ASN | 2101 | 30.108 | 5.263 | 69.440 | 1.00 | 28.75 |
| ATOM | 2789 | CG | ASN | 2101 | 29.324 | 4.023 | 69.771 | 1.00 | 29.79 |
| ATOM | 2790 | OD1 | ASN | 2101 | 28.821 | 3.328 | 68.875 | 1.00 | 31.16 |
| ATOM | 2791 | ND2 | ASN | 2101 | 29.208 | 3.730 | 71.062 | 1.00 | 29.82 |
| ATOM | 2792 | C | ASN | 2101 | 30.995 | 3.977 | 67.476 | 1.00 | 27.39 |
| ATOM | 2793 | O | ASN | 2101 | 31.828 | 3.351 | 68.131 | 1.00 | 26.65 |
| ATOM | 2794 | N | ASN | 2102 | 30.488 | 3.528 | 66.326 | 1.00 | 27.16 |
| ATOM | 2795 | CA | ASN | 2102 | 30.895 | 2.270 | 65.699 | 1.00 | 26.40 |
| ATOM | 2796 | CB | ASN | 2102 | 32.413 | 2.213 | 65.568 | 1.00 | 26.84 |
| ATOM | 2797 | CG | ASN | 2102 | 32.908 | 2.978 | 64.371 | 1.00 | 27.25 |
| ATOM | 2798 | OD1 | ASN | 2102 | 32.337 | 2.880 | 63.297 | 1.00 | 26.85 |
| ATOM | 2799 | ND2 | ASN | 2102 | 33.983 | 3.743 | 64.548 | 1.00 | 28.14 |
| ATOM | 2800 | C | ASN | 2102 | 30.398 | 0.967 | 66.306 | 1.00 | 26.54 |
| ATOM | 2801 | O | ASN | 2102 | 30.929 | −0.107 | 66.016 | 1.00 | 25.92 |
| ATOM | 2802 | N | TYR | 2103 | 29.393 | 1.054 | 67.164 | 1.00 | 26.22 |
| ATOM | 2803 | CA | TYR | 2103 | 28.815 | −0.142 | 67.732 | 1.00 | 25.44 |
| ATOM | 2804 | CB | TYR | 2103 | 28.775 | −0.050 | 69.250 | 1.00 | 25.93 |
| ATOM | 2805 | CG | TYR | 2103 | 30.106 | −0.330 | 69.923 | 1.00 | 26.38 |
| ATOM | 2806 | CD1 | TYR | 2103 | 30.426 | −1.608 | 70.394 | 1.00 | 26.02 |
| ATOM | 2807 | CE1 | TYR | 2103 | 31.622 | −1.845 | 71.067 | 1.00 | 25.86 |
| ATOM | 2808 | CD2 | TYR | 2103 | 31.026 | 0.695 | 70.134 | 1.00 | 26.61 |
| ATOM | 2809 | CE2 | TYR | 2103 | 32.222 | 0.467 | 70.801 | 1.00 | 25.99 |
| ATOM | 2810 | CZ | TYR | 2103 | 32.512 | −0.796 | 71.267 | 1.00 | 26.56 |
| ATOM | 2811 | OH | TYR | 2103 | 33.685 | −0.990 | 71.952 | 1.00 | 28.31 |
| ATOM | 2812 | C | TYR | 2103 | 27.410 | −0.208 | 67.147 | 1.00 | 25.48 |
| ATOM | 2813 | O | TYR | 2103 | 26.881 | 0.802 | 66.681 | 1.00 | 25.33 |
| ATOM | 2814 | N | ASN | 2104 | 26.825 | −1.398 | 67.132 | 1.00 | 25.61 |
| ATOM | 2815 | CA | ASN | 2104 | 25.482 | −1.575 | 66.609 | 1.00 | 25.53 |
| ATOM | 2816 | CB | ASN | 2104 | 25.369 | −2.894 | 65.861 | 1.00 | 26.04 |
| ATOM | 2817 | CG | ASN | 2104 | 25.956 | −2.845 | 64.460 | 1.00 | 27.07 |
| ATOM | 2818 | OD1 | ASN | 2104 | 26.605 | −1.875 | 64.062 | 1.00 | 28.87 |
| ATOM | 2819 | ND2 | ASN | 2104 | 25.737 | −3.917 | 63.706 | 1.00 | 26.67 |
| ATOM | 2820 | C | ASN | 2104 | 24.489 | −1.603 | 67.752 | 1.00 | 26.04 |
| ATOM | 2821 | O | ASN | 2104 | 24.835 | −1.972 | 68.873 | 1.00 | 26.56 |
| ATOM | 2822 | N | THR | 2105 | 23.259 | −1.193 | 67.472 | 1.00 | 26.42 |
| ATOM | 2823 | CA | THR | 2105 | 22.205 | −1.251 | 68.467 | 1.00 | 26.70 |
| ATOM | 2824 | CB | THR | 2105 | 21.664 | 0.127 | 68.859 | 1.00 | 26.84 |
| ATOM | 2825 | OG1 | THR | 2105 | 21.187 | 0.797 | 67.692 | 1.00 | 27.28 |
| ATOM | 2826 | CG2 | THR | 2105 | 22.736 | 0.946 | 69.550 | 1.00 | 26.53 |
| ATOM | 2827 | C | THR | 2105 | 21.088 | −2.013 | 67.784 | 1.00 | 27.60 |
| ATOM | 2828 | O | THR | 2105 | 21.021 | −2.058 | 66.549 | 1.00 | 27.98 |
| ATOM | 2829 | N | TYR | 2106 | 20.221 | −2.619 | 68.581 | 1.00 | 28.01 |
| ATOM | 2830 | CA | TYR | 2106 | 19.097 | −3.375 | 68.051 | 1.00 | 28.31 |
| ATOM | 2831 | CB | TYR | 2106 | 19.385 | −4.883 | 68.181 | 1.00 | 27.15 |
| ATOM | 2832 | CG | TYR | 2106 | 20.526 | −5.324 | 67.289 | 1.00 | 25.58 |
| ATOM | 2833 | CD1 | TYR | 2106 | 20.307 | −5.640 | 65.949 | 1.00 | 24.52 |
| ATOM | 2834 | CE1 | TYR | 2106 | 21.367 | −5.920 | 65.085 | 1.00 | 23.44 |
| ATOM | 2835 | CD2 | TYR | 2106 | 21.838 | −5.312 | 67.750 | 1.00 | 25.63 |
| ATOM | 2836 | CE2 | TYR | 2106 | 22.909 | −5.594 | 66.891 | 1.00 | 24.90 |
| ATOM | 2837 | CZ | TYR | 2106 | 22.664 | −5.894 | 65.560 | 1.00 | 23.78 |
| ATOM | 2838 | OH | TYR | 2106 | 23.724 | −6.157 | 64.711 | 1.00 | 23.43 |
| ATOM | 2839 | C | TYR | 2106 | 17.847 | −2.959 | 68.815 | 1.00 | 29.03 |
| ATOM | 2840 | O | TYR | 2106 | 17.654 | −3.317 | 69.972 | 1.00 | 28.35 |
| ATOM | 2841 | N | ARG | 2107 | 17.009 | −2.179 | 68.153 | 1.00 | 30.70 |
| ATOM | 2842 | CA | ARG | 2107 | 15.795 | −1.682 | 68.767 | 1.00 | 32.11 |
| ATOM | 2843 | CB | ARG | 2107 | 15.654 | −0.208 | 68.449 | 1.00 | 32.73 |
| ATOM | 2844 | CG | ARG | 2107 | 14.764 | 0.562 | 69.393 | 1.00 | 33.77 |
| ATOM | 2845 | CD | ARG | 2107 | 14.798 | 2.032 | 69.039 | 1.00 | 33.54 |
| ATOM | 2846 | NE | ARG | 2107 | 14.801 | 2.229 | 67.595 | 1.00 | 34.24 |
| ATOM | 2847 | CZ | ARG | 2107 | 14.074 | 3.140 | 66.967 | 1.00 | 35.51 |
| ATOM | 2848 | NH1 | ARG | 2107 | 13.275 | 3.940 | 67.661 | 1.00 | 36.69 |
| ATOM | 2849 | NH2 | ARG | 2107 | 14.153 | 3.261 | 65.649 | 1.00 | 35.73 |
| ATOM | 2850 | C | ARG | 2107 | 14.564 | −2.448 | 68.291 | 1.00 | 33.58 |
| ATOM | 2851 | O | ARG | 2107 | 14.391 | −2.709 | 67.093 | 1.00 | 33.94 |
| ATOM | 2852 | N | SER | 2108 | 13.715 | −2.818 | 69.240 | 1.00 | 34.22 |
| ATOM | 2853 | CA | SER | 2108 | 12.499 | −3.552 | 68.928 | 1.00 | 35.61 |
| ATOM | 2854 | CB | SER | 2108 | 11.717 | −3.832 | 70.202 | 1.00 | 35.04 |
| ATOM | 2855 | OG | SER | 2108 | 10.458 | −4.387 | 69.881 | 1.00 | 36.16 |
| ATOM | 2856 | C | SER | 2108 | 11.609 | −2.779 | 67.972 | 1.00 | 36.44 |
| ATOM | 2857 | O | SER | 2108 | 11.287 | −1.621 | 68.233 | 1.00 | 36.93 |
| ATOM | 2858 | N | ARG | 2109 | 11.207 | −3.407 | 66.870 | 1.00 | 37.57 |
| ATOM | 2859 | CA | ARG | 2109 | 10.338 | −2.717 | 65.929 | 1.00 | 39.12 |
| ATOM | 2860 | CB | ARG | 2109 | 10.314 | −3.405 | 64.564 | 1.00 | 39.94 |
| ATOM | 2861 | CG | ARG | 2109 | 9.299 | −2.727 | 63.634 | 1.00 | 42.56 |
| ATOM | 2862 | CD | ARG | 2109 | 9.530 | −2.959 | 62.153 | 1.00 | 43.76 |
| ATOM | 2863 | NE | ARG | 2109 | 9.089 | −4.268 | 61.689 | 1.00 | 46.95 |
| ATOM | 2864 | CZ | ARG | 2109 | 9.075 | −4.626 | 60.406 | 1.00 | 48.92 |
| ATOM | 2865 | NH1 | ARG | 2109 | 9.478 | −3.762 | 59.476 | 1.00 | 49.68 |
| ATOM | 2866 | NH2 | ARG | 2109 | 8.673 | −5.843 | 60.049 | 1.00 | 48.65 |
| ATOM | 2867 | C | ARG | 2109 | 8.919 | −2.637 | 66.487 | 1.00 | 39.72 |
| ATOM | 2868 | O | ARG | 2109 | 8.134 | −1.778 | 66.082 | 1.00 | 39.01 |
| ATOM | 2869 | N | LYS | 2110 | 8.601 | −3.529 | 67.425 | 1.00 | 40.31 |
| ATOM | 2870 | CA | LYS | 2110 | 7.283 | −3.532 | 68.032 | 1.00 | 40.94 |
| ATOM | 2871 | CB | LYS | 2110 | 6.870 | −4.936 | 68.478 | 1.00 | 41.71 |
| ATOM | 2872 | CG | LYS | 2110 | 5.380 | −5.016 | 68.785 | 1.00 | 43.04 |
| ATOM | 2873 | CD | LYS | 2110 | 4.881 | −6.441 | 68.959 | 1.00 | 44.52 |
| ATOM | 2874 | CE | LYS | 2110 | 3.372 | −6.473 | 69.197 | 1.00 | 44.39 |
| ATOM | 2875 | NZ | LYS | 2110 | 2.624 | −5.898 | 68.039 | 1.00 | 44.75 |
| ATOM | 2876 | C | LYS | 2110 | 7.253 | −2.574 | 69.211 | 1.00 | 40.76 |
| ATOM | 2877 | O | LYS | 2110 | 6.349 | −1.747 | 69.315 | 1.00 | 41.46 |
| ATOM | 2878 | N | TYR | 2111 | 8.246 | −2.679 | 70.087 | 1.00 | 40.63 |
| ATOM | 2879 | CA | TYR | 2111 | 8.362 | −1.808 | 71.263 | 1.00 | 40.00 |
| ATOM | 2880 | CB | TYR | 2111 | 8.662 | −2.676 | 72.486 | 1.00 | 40.34 |
| ATOM | 2881 | CG | TYR | 2111 | 7.733 | −3.868 | 72.559 | 1.00 | 40.79 |
| ATOM | 2882 | CD1 | TYR | 2111 | 6.377 | −3.702 | 72.841 | 1.00 | 40.71 |
| ATOM | 2883 | CE1 | TYR | 2111 | 5.496 | −4.784 | 72.819 | 1.00 | 41.25 |
| ATOM | 2884 | CD2 | TYR | 2111 | 8.190 | −5.154 | 72.261 | 1.00 | 41.12 |
| ATOM | 2885 | CE2 | TYR | 2111 | 7.317 | −6.247 | 72.237 | 1.00 | 41.36 |
| ATOM | 2886 | CZ | TYR | 2111 | 5.971 | −6.053 | 72.516 | 1.00 | 41.75 |
| ATOM | 2887 | OH | TYR | 2111 | 5.097 | −7.123 | 72.491 | 1.00 | 42.21 |
| ATOM | 2888 | C | TYR | 2111 | 9.505 | −0.837 | 70.957 | 1.00 | 39.35 |
| ATOM | 2889 | O | TYR | 2111 | 10.584 | −0.908 | 71.540 | 1.00 | 39.73 |
| ATOM | 2890 | N | THR | 2112 | 9.221 | 0.065 | 70.022 | 1.00 | 38.73 |
| ATOM | 2891 | CA | THR | 2112 | 10.154 | 1.057 | 69.501 | 1.00 | 38.68 |
| ATOM | 2892 | CB | THR | 2112 | 9.409 | 2.127 | 68.666 | 1.00 | 38.91 |
| ATOM | 2893 | OG1 | THR | 2112 | 8.434 | 2.781 | 69.487 | 1.00 | 39.96 |
| ATOM | 2894 | CG2 | THR | 2112 | 8.731 | 1.496 | 67.460 | 1.00 | 38.24 |
| ATOM | 2895 | C | THR | 2112 | 11.125 | 1.800 | 70.400 | 1.00 | 38.37 |
| ATOM | 2896 | O | THR | 2112 | 12.044 | 2.438 | 69.890 | 1.00 | 39.14 |
| ATOM | 2897 | N | SER | 2113 | 10.951 | 1.760 | 71.712 | 1.00 | 38.11 |
| ATOM | 2898 | CA | SER | 2113 | 11.900 | 2.477 | 72.555 | 1.00 | 38.34 |
| ATOM | 2899 | CB | SER | 2113 | 11.189 | 3.548 | 73.404 | 1.00 | 39.61 |
| ATOM | 2900 | OG | SER | 2113 | 10.058 | 3.032 | 74.079 | 1.00 | 42.34 |
| ATOM | 2901 | C | SER | 2113 | 12.743 | 1.557 | 73.427 | 1.00 | 37.71 |
| ATOM | 2902 | O | SER | 2113 | 13.476 | 2.016 | 74.301 | 1.00 | 37.56 |
| ATOM | 2903 | N | TRP | 2114 | 12.658 | 0.257 | 73.167 | 1.00 | 37.04 |
| ATOM | 2904 | CA | TRP | 2114 | 13.434 | −0.718 | 73.928 | 1.00 | 36.46 |
| ATOM | 2905 | CB | TRP | 2114 | 12.522 | −1.806 | 74.472 | 1.00 | 38.16 |
| ATOM | 2906 | CG | TRP | 2114 | 11.488 | −1.314 | 75.426 | 1.00 | 40.57 |
| ATOM | 2907 | CD2 | TRP | 2114 | 10.405 | −2.075 | 75.958 | 1.00 | 41.10 |
| ATOM | 2908 | CE2 | TRP | 2114 | 9.754 | −1.267 | 76.911 | 1.00 | 41.79 |
| ATOM | 2909 | CE3 | TRP | 2114 | 9.923 | −3.372 | 75.725 | 1.00 | 41.40 |
| ATOM | 2910 | CD1 | TRP | 2114 | 11.447 | −0.095 | 76.051 | 1.00 | 40.98 |
| ATOM | 2911 | NE1 | TRP | 2114 | 10.410 | −0.062 | 76.947 | 1.00 | 41.41 |
| ATOM | 2912 | CZ2 | TRP | 2114 | 8.647 | −1.712 | 77.636 | 1.00 | 42.61 |
| ATOM | 2913 | CZ3 | TRP | 2114 | 8.822 | −3.816 | 76.445 | 1.00 | 42.31 |
| ATOM | 2914 | CH2 | TRP | 2114 | 8.197 | −2.988 | 77.389 | 1.00 | 42.61 |
| ATOM | 2915 | C | TRP | 2114 | 14.531 | −1.370 | 73.095 | 1.00 | 35.03 |
| ATOM | 2916 | O | TRP | 2114 | 14.313 | −1.731 | 71.943 | 1.00 | 34.30 |
| ATOM | 2917 | N | TYR | 2115 | 15.706 | −1.530 | 73.695 | 1.00 | 33.60 |
| ATOM | 2918 | CA | TYR | 2115 | 16.850 | −2.126 | 73.016 | 1.00 | 31.96 |
| ATOM | 2919 | CB | TYR | 2115 | 18.102 | −1.261 | 73.192 | 1.00 | 31.76 |
| ATOM | 2920 | CG | TYR | 2115 | 18.045 | 0.119 | 72.604 | 1.00 | 31.70 |
| ATOM | 2921 | CD1 | TYR | 2115 | 17.365 | 1.144 | 73.247 | 1.00 | 31.71 |
| ATOM | 2922 | CE1 | TYR | 2115 | 17.299 | 2.417 | 72.692 | 1.00 | 32.06 |
| ATOM | 2923 | CD2 | TYR | 2115 | 18.666 | 0.399 | 71.387 | 1.00 | 32.06 |
| ATOM | 2924 | CE2 | TYR | 2115 | 18.606 | 1.661 | 70.821 | 1.00 | 32.34 |
| ATOM | 2925 | CZ | TYR | 2115 | 17.918 | 2.667 | 71.475 | 1.00 | 32.58 |
| ATOM | 2926 | OH | TYR | 2115 | 17.810 | 3.912 | 70.894 | 1.00 | 33.03 |
| ATOM | 2927 | C | TYR | 2115 | 17.212 | −3.504 | 73.551 | 1.00 | 31.50 |
| ATOM | 2928 | O | TYR | 2115 | 16.955 | −3.825 | 74.717 | 1.00 | 32.03 |
| ATOM | 2929 | N | VAL | 2116 | 17.831 | −4.311 | 72.698 | 1.00 | 30.32 |
| ATOM | 2930 | CA | VAL | 2116 | 18.300 | −5.616 | 73.121 | 1.00 | 29.98 |
| ATOM | 2931 | CB | VAL | 2116 | 18.778 | −6.458 | 71.928 | 1.00 | 29.14 |
| ATOM | 2932 | CG1 | VAL | 2116 | 19.548 | −7.665 | 72.407 | 1.00 | 28.14 |
| ATOM | 2933 | CG2 | VAL | 2116 | 17.597 | −6.889 | 71.114 | 1.00 | 29.83 |
| ATOM | 2934 | C | VAL | 2116 | 19.495 | −5.226 | 73.988 | 1.00 | 30.87 |
| ATOM | 2935 | O | VAL | 2116 | 20.250 | −4.329 | 73.624 | 1.00 | 31.54 |
| ATOM | 2936 | N | ALA | 2117 | 19.669 | −5.876 | 75.133 | 1.00 | 31.62 |
| ATOM | 2937 | CA | ALA | 2117 | 20.768 | −5.514 | 76.014 | 1.00 | 31.53 |
| ATOM | 2938 | CB | ALA | 2117 | 20.398 | −4.269 | 76.777 | 1.00 | 31.35 |
| ATOM | 2939 | C | ALA | 2117 | 21.163 | −6.601 | 76.997 | 1.00 | 32.19 |
| ATOM | 2940 | O | ALA | 2117 | 20.344 | −7.442 | 77.374 | 1.00 | 32.01 |
| ATOM | 2941 | N | LEU | 2118 | 22.429 | −6.568 | 77.411 | 1.00 | 32.78 |
| ATOM | 2942 | CA | LEU | 2118 | 22.968 | −7.518 | 78.383 | 1.00 | 33.96 |
| ATOM | 2943 | CB | LEU | 2118 | 24.081 | −8.379 | 77.768 | 1.00 | 31.56 |
| ATOM | 2944 | CG | LEU | 2118 | 23.711 | −9.308 | 76.605 | 1.00 | 30.25 |
| ATOM | 2945 | CD1 | LEU | 2118 | 24.931 | −10.072 | 76.196 | 1.00 | 28.61 |
| ATOM | 2946 | CD2 | LEU | 2118 | 22.574 | −10.260 | 76.999 | 1.00 | 28.40 |
| ATOM | 2947 | C | LEU | 2118 | 23.536 | −6.749 | 79.566 | 1.00 | 35.73 |
| ATOM | 2948 | O | LEU | 2118 | 24.145 | −5.698 | 79.384 | 1.00 | 36.49 |
| ATOM | 2949 | N | LYS | 2119 | 23.320 | −7.267 | 80.775 | 1.00 | 37.76 |
| ATOM | 2950 | CA | LYS | 2119 | 23.826 | −6.640 | 81.990 | 1.00 | 38.95 |
| ATOM | 2951 | CB | LYS | 2119 | 23.029 | −7.102 | 83.214 | 1.00 | 38.97 |
| ATOM | 2952 | CG | LYS | 2119 | 21.517 | −6.882 | 83.164 | 1.00 | 39.95 |
| ATOM | 2953 | CD | LYS | 2119 | 20.902 | −7.148 | 84.544 | 1.00 | 40.37 |
| ATOM | 2954 | CE | LYS | 2119 | 19.415 | −7.560 | 84.493 | 1.00 | 42.09 |
| ATOM | 2955 | NZ | LYS | 2119 | 18.392 | −6.492 | 84.201 | 1.00 | 42.91 |
| ATOM | 2956 | C | LYS | 2119 | 25.277 | −7.067 | 82.163 | 1.00 | 40.31 |
| ATOM | 2957 | O | LYS | 2119 | 25.757 | −7.952 | 81.462 | 1.00 | 39.96 |
| ATOM | 2958 | N | ARG | 2120 | 25.977 | −6.445 | 83.102 | 1.00 | 42.63 |
| ATOM | 2959 | CA | ARG | 2120 | 27.368 | −6.792 | 83.351 | 1.00 | 45.00 |
| ATOM | 2960 | CB | ARG | 2120 | 27.993 | −5.786 | 84.308 | 1.00 | 47.63 |
| ATOM | 2961 | CG | ARG | 2120 | 28.024 | −4.385 | 83.760 | 1.00 | 51.89 |
| ATOM | 2962 | CD | ARG | 2120 | 28.543 | −3.400 | 84.779 | 1.00 | 55.64 |
| ATOM | 2963 | NE | ARG | 2120 | 28.807 | −2.102 | 84.161 | 1.00 | 59.93 |
| ATOM | 2964 | CZ | ARG | 2120 | 29.147 | −0.999 | 84.828 | 1.00 | 62.27 |
| ATOM | 2965 | NH1 | ARG | 2120 | 29.266 | −1.020 | 86.156 | 1.00 | 62.86 |
| ATOM | 2966 | NH2 | ARG | 2120 | 29.378 | 0.131 | 84.162 | 1.00 | 63.23 |
| ATOM | 2967 | C | ARG | 2120 | 27.489 | −8.194 | 83.933 | 1.00 | 44.95 |
| ATOM | 2968 | O | ARG | 2120 | 28.584 | −8.747 | 84.014 | 1.00 | 45.89 |
| ATOM | 2969 | N | THR | 2121 | 26.358 | −8.763 | 84.334 | 1.00 | 44.82 |
| ATOM | 2970 | CA | THR | 2121 | 26.311 | −10.103 | 84.912 | 1.00 | 44.65 |
| ATOM | 2971 | CB | THR | 2121 | 25.084 | −10.269 | 85.798 | 1.00 | 44.06 |
| ATOM | 2972 | OG1 | THR | 2121 | 23.912 | −9.927 | 85.047 | 1.00 | 43.55 |
| ATOM | 2973 | CG2 | THR | 2121 | 25.181 | −9.383 | 87.013 | 1.00 | 43.86 |
| ATOM | 2974 | C | THR | 2121 | 26.229 | −11.187 | 83.846 | 1.00 | 44.98 |
| ATOM | 2975 | O | THR | 2121 | 26.387 | −12.369 | 84.141 | 1.00 | 46.06 |
| ATOM | 2976 | N | GLY | 2122 | 25.970 | −10.787 | 82.610 | 1.00 | 44.69 |
| ATOM | 2977 | CA | GLY | 2122 | 25.847 | −11.759 | 81.544 | 1.00 | 44.75 |
| ATOM | 2978 | C | GLY | 2122 | 24.385 | −12.117 | 81.329 | 1.00 | 44.90 |
| ATOM | 2979 | O | GLY | 2122 | 24.051 | −12.890 | 80.435 | 1.00 | 45.54 |
| ATOM | 2980 | N | GLN | 2123 | 23.511 | −11.567 | 82.163 | 1.00 | 44.47 |
| ATOM | 2981 | CA | GLN | 2123 | 22.086 | −11.819 | 82.035 | 1.00 | 44.64 |
| ATOM | 2982 | CB | GLN | 2123 | 21.402 | −11.724 | 83.386 | 1.00 | 46.21 |
| ATOM | 2983 | CG | GLN | 2123 | 22.029 | −12.550 | 84.473 | 1.00 | 48.48 |
| ATOM | 2984 | CD | GLN | 2123 | 21.442 | −12.205 | 85.827 | 1.00 | 50.36 |
| ATOM | 2985 | OE1 | GLN | 2123 | 21.569 | −11.067 | 86.308 | 1.00 | 50.59 |
| ATOM | 2986 | NE2 | GLN | 2123 | 20.780 | −13.181 | 86.448 | 1.00 | 51.47 |
| ATOM | 2987 | C | GLN | 2123 | 21.543 | −10.717 | 81.146 | 1.00 | 44.21 |
| ATOM | 2988 | O | GLN | 2123 | 22.042 | −9.596 | 81.178 | 1.00 | 43.82 |
| ATOM | 2989 | N | TYR | 2124 | 20.520 | −11.011 | 80.356 | 1.00 | 43.41 |
| ATOM | 2990 | CA | TYR | 2124 | 19.997 | −9.972 | 79.491 | 1.00 | 42.74 |
| ATOM | 2991 | CB | TYR | 2124 | 19.009 | −10.546 | 78.469 | 1.00 | 42.17 |
| ATOM | 2992 | CG | TYR | 2124 | 17.638 | −10.877 | 79.001 | 1.00 | 42.56 |
| ATOM | 2993 | CD1 | TYR | 2124 | 16.698 | −9.876 | 79.238 | 1.00 | 42.31 |
| ATOM | 2994 | CE1 | TYR | 2124 | 15.427 | −10.186 | 79.696 | 1.00 | 42.79 |
| ATOM | 2995 | CD2 | TYR | 2124 | 17.268 | −12.199 | 79.242 | 1.00 | 42.45 |
| ATOM | 2996 | CE2 | TYR | 2124 | 16.006 | −12.514 | 79.700 | 1.00 | 42.40 |
| ATOM | 2997 | CZ | TYR | 2124 | 15.088 | −11.509 | 79.926 | 1.00 | 42.56 |
| ATOM | 2998 | OH | TYR | 2124 | 13.834 | −11.830 | 80.388 | 1.00 | 42.62 |
| ATOM | 2999 | C | TYR | 2124 | 19.348 | −8.912 | 80.360 | 1.00 | 42.47 |
| ATOM | 3000 | O | TYR | 2124 | 19.009 | −9.171 | 81.509 | 1.00 | 42.50 |
| ATOM | 3001 | N | LYS | 2125 | 19.200 | −7.712 | 79.818 | 1.00 | 41.99 |
| ATOM | 3002 | CA | LYS | 2125 | 18.591 | −6.618 | 80.553 | 1.00 | 41.43 |
| ATOM | 3003 | CB | LYS | 2125 | 19.484 | −5.384 | 80.480 | 1.00 | 41.27 |
| ATOM | 3004 | CG | LYS | 2125 | 18.895 | −4.149 | 81.130 | 1.00 | 40.16 |
| ATOM | 3005 | CD | LYS | 2125 | 19.758 | −2.937 | 80.870 | 1.00 | 39.70 |
| ATOM | 3006 | CE | LYS | 2125 | 20.114 | −2.249 | 82.165 | 1.00 | 39.65 |
| ATOM | 3007 | NZ | LYS | 2125 | 20.858 | −0.988 | 81.938 | 1.00 | 39.01 |
| ATOM | 3008 | C | LYS | 2125 | 17.230 | −6.297 | 79.951 | 1.00 | 41.80 |
| ATOM | 3009 | O | LYS | 2125 | 17.089 | −6.193 | 78.730 | 1.00 | 41.73 |
| ATOM | 3010 | N | LEU | 2126 | 16.224 | −6.146 | 80.806 | 1.00 | 42.08 |
| ATOM | 3011 | CA | LEU | 2126 | 14.885 | −5.841 | 80.331 | 1.00 | 42.35 |
| ATOM | 3012 | CB | LEU | 2126 | 13.943 | −5.579 | 81.503 | 1.00 | 43.27 |
| ATOM | 3013 | CG | LEU | 2126 | 13.366 | −6.802 | 82.219 | 1.00 | 44.21 |
| ATOM | 3014 | CD1 | LEU | 2126 | 12.212 | −6.356 | 83.095 | 1.00 | 43.89 |
| ATOM | 3015 | CD2 | LEU | 2126 | 12.870 | −7.824 | 81.199 | 1.00 | 44.37 |
| ATOM | 3016 | C | LEU | 2126 | 14.878 | −4.643 | 79.402 | 1.00 | 42.64 |
| ATOM | 3017 | O | LEU | 2126 | 15.425 | −3.590 | 79.727 | 1.00 | 42.44 |
| ATOM | 3018 | N | GLY | 2127 | 14.248 | −4.810 | 78.244 | 1.00 | 42.68 |
| ATOM | 3019 | CA | GLY | 2127 | 14.174 | −3.727 | 77.283 | 1.00 | 42.96 |
| ATOM | 3020 | C | GLY | 2127 | 13.525 | −2.502 | 77.889 | 1.00 | 43.02 |
| ATOM | 3021 | O | GLY | 2127 | 13.866 | −1.369 | 77.555 | 1.00 | 43.21 |
| ATOM | 3022 | N | SER | 2128 | 12.585 | −2.734 | 78.795 | 1.00 | 43.02 |
| ATOM | 3023 | CA | SER | 2128 | 11.887 | −1.641 | 79.444 | 1.00 | 43.53 |
| ATOM | 3024 | CB | SER | 2128 | 10.748 | −2.189 | 80.309 | 1.00 | 43.90 |
| ATOM | 3025 | OG | SER | 2128 | 11.235 | −3.063 | 81.316 | 1.00 | 44.04 |
| ATOM | 3026 | C | SER | 2128 | 12.839 | −0.802 | 80.295 | 1.00 | 43.82 |
| ATOM | 3027 | O | SER | 2128 | 12.518 | 0.324 | 80.670 | 1.00 | 43.82 |
| ATOM | 3028 | N | LYS | 2129 | 14.008 | −1.350 | 80.600 | 1.00 | 43.76 |
| ATOM | 3029 | CA | LYS | 2129 | 14.987 | −0.627 | 81.402 | 1.00 | 44.27 |
| ATOM | 3030 | CB | LYS | 2129 | 15.531 | −1.518 | 82.515 | 1.00 | 45.58 |
| ATOM | 3031 | CG | LYS | 2129 | 14.580 | −1.732 | 83.674 | 1.00 | 47.07 |
| ATOM | 3032 | CD | LYS | 2129 | 15.205 | −2.684 | 84.684 | 1.00 | 48.78 |
| ATOM | 3033 | CE | LYS | 2129 | 14.269 | −2.977 | 85.849 | 1.00 | 49.97 |
| ATOM | 3034 | NZ | LYS | 2129 | 14.775 | −4.126 | 86.667 | 1.00 | 51.11 |
| ATOM | 3035 | C | LYS | 2129 | 16.160 | −0.099 | 80.577 | 1.00 | 43.94 |
| ATOM | 3036 | O | LYS | 2129 | 17.108 | 0.450 | 81.130 | 1.00 | 43.60 |
| ATOM | 3037 | N | THR | 2130 | 16.100 | −0.263 | 79.259 | 1.00 | 43.35 |
| ATOM | 3038 | CA | THR | 2130 | 17.183 | 0.202 | 78.407 | 1.00 | 42.94 |
| ATOM | 3039 | CB | THR | 2130 | 17.345 | −0.697 | 77.155 | 1.00 | 42.84 |
| ATOM | 3040 | OG1 | THR | 2130 | 16.156 | −0.655 | 76.358 | 1.00 | 42.17 |
| ATOM | 3041 | CG2 | THR | 2130 | 17.613 | −2.126 | 77.576 | 1.00 | 43.08 |
| ATOM | 3042 | C | THR | 2130 | 16.982 | 1.648 | 77.973 | 1.00 | 42.95 |
| ATOM | 3043 | O | THR | 2130 | 15.902 | 2.211 | 78.148 | 1.00 | 43.02 |
| ATOM | 3044 | N | GLY | 2131 | 18.036 | 2.239 | 77.419 | 1.00 | 42.70 |
| ATOM | 3045 | CA | GLY | 2131 | 17.978 | 3.615 | 76.965 | 1.00 | 42.23 |
| ATOM | 3046 | C | GLY | 2131 | 19.143 | 3.897 | 76.043 | 1.00 | 42.39 |
| ATOM | 3047 | O | GLY | 2131 | 20.099 | 3.127 | 76.022 | 1.00 | 43.27 |
| ATOM | 3048 | N | PRO | 2132 | 19.109 | 5.006 | 75.291 | 1.00 | 42.14 |
| ATOM | 3049 | CD | PRO | 2132 | 18.107 | 6.068 | 75.461 | 1.00 | 41.69 |
| ATOM | 3050 | CA | PRO | 2132 | 20.146 | 5.430 | 74.340 | 1.00 | 41.69 |
| ATOM | 3051 | CB | PRO | 2132 | 19.631 | 6.783 | 73.846 | 1.00 | 41.69 |
| ATOM | 3052 | CG | PRO | 2132 | 18.166 | 6.752 | 74.140 | 1.00 | 41.64 |
| ATOM | 3053 | C | PRO | 2132 | 21.542 | 5.578 | 74.935 | 1.00 | 41.55 |
| ATOM | 3054 | O | PRO | 2132 | 22.541 | 5.213 | 74.315 | 1.00 | 41.92 |
| ATOM | 3055 | N | GLY | 2133 | 21.601 | 6.140 | 76.139 | 1.00 | 41.22 |
| ATOM | 3056 | CA | GLY | 2133 | 22.877 | 6.369 | 76.781 | 1.00 | 40.02 |
| ATOM | 3057 | C | GLY | 2133 | 23.420 | 5.231 | 77.612 | 1.00 | 39.88 |
| ATOM | 3058 | O | GLY | 2133 | 24.174 | 5.460 | 78.562 | 1.00 | 39.33 |
| ATOM | 3059 | N | GLN | 2134 | 23.060 | 3.999 | 77.271 | 1.00 | 39.42 |
| ATOM | 3060 | CA | GLN | 2134 | 23.552 | 2.863 | 78.041 | 1.00 | 38.56 |
| ATOM | 3061 | CB | GLN | 2134 | 22.390 | 1.973 | 78.493 | 1.00 | 38.82 |
| ATOM | 3062 | CG | GLN | 2134 | 21.329 | 2.697 | 79.308 | 1.00 | 39.65 |
| ATOM | 3063 | CD | GLN | 2134 | 20.161 | 1.797 | 79.681 | 1.00 | 39.65 |
| ATOM | 3064 | OE1 | GLN | 2134 | 19.184 | 2.243 | 80.286 | 1.00 | 40.44 |
| ATOM | 3065 | NE2 | GLN | 2134 | 20.256 | 0.528 | 79.321 | 1.00 | 39.76 |
| ATOM | 3066 | C | GLN | 2134 | 24.567 | 2.017 | 77.283 | 1.00 | 37.84 |
| ATOM | 3067 | O | GLN | 2134 | 24.463 | 1.826 | 76.074 | 1.00 | 37.90 |
| ATOM | 3068 | N | LYS | 2135 | 25.548 | 1.513 | 78.019 | 1.00 | 36.94 |
| ATOM | 3069 | CA | LYS | 2135 | 26.591 | 0.676 | 77.459 | 1.00 | 36.45 |
| ATOM | 3070 | CB | LYS | 2135 | 27.756 | 0.560 | 78.442 | 1.00 | 36.60 |
| ATOM | 3071 | CG | LYS | 2135 | 28.903 | −0.308 | 77.933 | 1.00 | 38.16 |
| ATOM | 3072 | CD | LYS | 2135 | 29.949 | −0.578 | 79.008 | 1.00 | 39.10 |
| ATOM | 3073 | CE | LYS | 2135 | 30.506 | 0.708 | 79.604 | 1.00 | 40.18 |
| ATOM | 3074 | NZ | LYS | 2135 | 31.622 | 0.436 | 80.552 | 1.00 | 40.84 |
| ATOM | 3075 | C | LYS | 2135 | 26.054 | −0.718 | 77.157 | 1.00 | 36.15 |
| ATOM | 3076 | O | LYS | 2135 | 26.604 | −1.438 | 76.315 | 1.00 | 37.31 |
| ATOM | 3077 | N | ALA | 2136 | 24.971 | −1.086 | 77.834 | 1.00 | 34.85 |
| ATOM | 3078 | CA | ALA | 2136 | 24.371 | −2.403 | 77.685 | 1.00 | 33.86 |
| ATOM | 3079 | CB | ALA | 2136 | 23.375 | −2.638 | 78.825 | 1.00 | 33.84 |
| ATOM | 3080 | C | ALA | 2136 | 23.701 | −2.676 | 76.340 | 1.00 | 32.77 |
| ATOM | 3081 | O | ALA | 2136 | 23.507 | −3.833 | 75.972 | 1.00 | 32.53 |
| ATOM | 3082 | N | ILE | 2137 | 23.369 | −1.624 | 75.597 | 1.00 | 31.86 |
| ATOM | 3083 | CA | ILE | 2137 | 22.695 | −1.803 | 74.316 | 1.00 | 31.05 |
| ATOM | 3084 | CB | ILE | 2137 | 21.650 | −0.684 | 74.088 | 1.00 | 31.33 |
| ATOM | 3085 | CG2 | ILE | 2137 | 20.759 | −0.546 | 75.315 | 1.00 | 31.19 |
| ATOM | 3086 | CG1 | ILE | 2137 | 22.350 | 0.655 | 73.846 | 1.00 | 32.27 |
| ATOM | 3087 | CD1 | ILE | 2137 | 21.397 | 1.772 | 73.403 | 1.00 | 32.21 |
| ATOM | 3088 | C | ILE | 2137 | 23.641 | −1.845 | 73.114 | 1.00 | 30.94 |
| ATOM | 3089 | O | ILE | 2137 | 23.219 | −2.116 | 71.983 | 1.00 | 30.58 |
| ATOM | 3090 | N | LEU | 2138 | 24.925 | −1.603 | 73.367 | 1.00 | 30.51 |
| ATOM | 3091 | CA | LEU | 2138 | 25.928 | −1.576 | 72.303 | 1.00 | 29.42 |
| ATOM | 3092 | CB | LEU | 2138 | 27.024 | −0.563 | 72.665 | 1.00 | 27.55 |
| ATOM | 3093 | CG | LEU | 2138 | 26.484 | 0.837 | 72.996 | 1.00 | 26.99 |
| ATOM | 3094 | CD1 | LEU | 2138 | 27.604 | 1.726 | 73.505 | 1.00 | 26.16 |
| ATOM | 3095 | CD2 | LEU | 2138 | 25.832 | 1.448 | 71.779 | 1.00 | 24.73 |
| ATOM | 3096 | C | LEU | 2138 | 26.558 | −2.933 | 71.997 | 1.00 | 29.46 |
| ATOM | 3097 | O | LEU | 2138 | 27.076 | −3.608 | 72.888 | 1.00 | 28.90 |
| ATOM | 3098 | N | PHE | 2139 | 26.526 | −3.323 | 70.726 | 1.00 | 28.91 |
| ATOM | 3099 | CA | PHE | 2139 | 27.110 | −4.595 | 70.335 | 1.00 | 28.76 |
| ATOM | 3100 | CB | PHE | 2139 | 26.014 | −5.608 | 69.980 | 1.00 | 28.14 |
| ATOM | 3101 | CG | PHE | 2139 | 25.109 | −5.946 | 71.130 | 1.00 | 27.61 |
| ATOM | 3102 | CD1 | PHE | 2139 | 24.065 | −5.099 | 71.488 | 1.00 | 27.76 |
| ATOM | 3103 | CD2 | PHE | 2139 | 25.321 | −7.095 | 71.882 | 1.00 | 27.03 |
| ATOM | 3104 | CE1 | PHE | 2139 | 23.242 | −5.390 | 72.581 | 1.00 | 27.04 |
| ATOM | 3105 | CE2 | PEE | 2139 | 24.504 | −7.394 | 72.975 | 1.00 | 26.53 |
| ATOM | 3106 | CZ | PHE | 2139 | 23.465 | −6.541 | 73.323 | 1.00 | 26.62 |
| ATOM | 3107 | C | PEE | 2139 | 28.075 | −4.453 | 69.175 | 1.00 | 29.14 |
| ATOM | 3108 | O | PHE | 2139 | 27.873 | −3.641 | 68.275 | 1.00 | 29.62 |
| ATOM | 3109 | N | LEU | 2140 | 29.141 | −5.235 | 69.211 | 1.00 | 29.27 |
| ATOM | 3110 | CA | LEU | 2140 | 30.120 | −5.208 | 68.140 | 1.00 | 30.55 |
| ATOM | 3111 | CB | LEU | 2140 | 31.537 | −5.067 | 68.701 | 1.00 | 30.31 |
| ATOM | 3112 | CG | LEU | 2140 | 32.691 | −5.009 | 67.699 | 1.00 | 30.06 |
| ATOM | 3113 | CD1 | LEU | 2140 | 32.535 | −3.783 | 66.806 | 1.00 | 30.98 |
| ATOM | 3114 | CD2 | LEU | 2140 | 34.011 | −4.963 | 68.442 | 1.00 | 28.70 |
| ATOM | 3115 | C | LEU | 2140 | 29.988 | −6.517 | 67.359 | 1.00 | 31.65 |
| ATOM | 3116 | O | LEU | 2140 | 30.258 | −7.600 | 67.880 | 1.00 | 31.53 |
| ATOM | 3117 | N | PRO | 2141 | 29.540 | −6.437 | 66.102 | 1.00 | 32.57 |
| ATOM | 3118 | CD | PRO | 2141 | 29.008 | −5.282 | 65.359 | 1.00 | 32.72 |
| ATOM | 3119 | CA | PRO | 2141 | 29.402 | −7.668 | 65.325 | 1.00 | 34.30 |
| ATOM | 3120 | CB | PRO | 2141 | 28.539 | −7.232 | 64.139 | 1.00 | 33.67 |
| ATOM | 3121 | CG | PRO | 2141 | 28.926 | −5.814 | 63.956 | 1.00 | 33.62 |
| ATOM | 3122 | C | PRO | 2141 | 30.754 | −8.218 | 64.906 | 1.00 | 35.55 |
| ATOM | 3123 | O | PRO | 2141 | 31.623 | −7.480 | 64.452 | 1.00 | 35.53 |
| ATOM | 3124 | N | MET | 2142 | 30.928 | −9.521 | 65.074 | 1.00 | 37.33 |
| ATOM | 3125 | CA | MET | 2142 | 32.176 | −10.179 | 64.715 | 1.00 | 39.79 |
| ATOM | 3126 | CB | MET | 2142 | 32.943 | −10.565 | 65.967 | 1.00 | 39.15 |
| ATOM | 3127 | CG | MET | 2142 | 33.135 | −9.422 | 66.920 | 1.00 | 39.66 |
| ATOM | 3128 | SD | MET | 2142 | 34.054 | −9.913 | 68.362 | 1.00 | 40.40 |
| ATOM | 3129 | CE | MET | 2142 | 35.704 | −9.868 | 67.690 | 1.00 | 40.72 |
| ATOM | 3130 | C | MET | 2142 | 31.846 | −11.425 | 63.924 | 1.00 | 42.09 |
| ATOM | 3131 | O | MET | 2142 | 30.827 | −12.072 | 64.175 | 1.00 | 42.54 |
| ATOM | 3132 | N | SER | 2143 | 32.694 | −11.771 | 62.965 | 1.00 | 44.91 |
| ATOM | 3133 | CA | SER | 2143 | 32.431 | −12.961 | 62.172 | 1.00 | 47.68 |
| ATOM | 3134 | CB | SER | 2143 | 33.448 | −13.114 | 61.044 | 1.00 | 48.00 |
| ATOM | 3135 | OG | SER | 2143 | 34.728 | −13.440 | 61.551 | 1.00 | 48.82 |
| ATOM | 3136 | C | SER | 2143 | 32.495 | −14.171 | 63.090 | 1.00 | 49.84 |
| ATOM | 3137 | O | SER | 2143 | 33.140 | −14.141 | 64.147 | 1.00 | 50.20 |
| ATOM | 3138 | N | ALA | 2144 | 31.803 | −15.231 | 62.694 | 1.00 | 52.08 |
| ATOM | 3139 | CA | ALA | 2144 | 31.783 | −16.450 | 63.483 | 1.00 | 54.70 |
| ATOM | 3140 | CB | ALA | 2144 | 30.354 | −16.952 | 63.624 | 1.00 | 54.18 |
| ATOM | 3141 | C | ALA | 2144 | 32.646 | −17.493 | 62.784 | 1.00 | 56.50 |
| ATOM | 3142 | O | ALA | 2144 | 32.246 | −18.048 | 61.762 | 1.00 | 57.37 |
| ATOM | 3143 | N | LYS | 2145 | 33.830 | −17.747 | 63.333 | 1.00 | 58.18 |
| ATOM | 3144 | CA | LYS | 2145 | 34.758 | −18.714 | 62.759 | 1.00 | 59.52 |
| ATOM | 3145 | CB | LYS | 2145 | 35.673 | −18.023 | 61.744 | 1.00 | 60.81 |
| ATOM | 3146 | CG | LYS | 2145 | 34.947 | −17.467 | 60.526 | 1.00 | 62.29 |
| ATOM | 3147 | CD | LYS | 2145 | 35.500 | −18.086 | 59.251 | 1.00 | 63.58 |
| ATOM | 3148 | CE | LYS | 2145 | 34.704 | −17.657 | 58.029 | 1.00 | 65.22 |
| ATOM | 3149 | NZ | LYS | 2145 | 35.178 | −18.363 | 56.796 | 1.00 | 66.48 |
| ATOM | 3150 | C | LYS | 2145 | 35.600 | −19.356 | 63.857 | 1.00 | 59.80 |
| ATOM | 3151 | O | LYS | 2145 | 35.008 | −20.141 | 64.630 | 1.00 | 60.14 |
| ATOM | 3152 | C | GLY | 3015 | 39.270 | 22.850 | 54.776 | 1.00 | 43.87 |
| ATOM | 3153 | O | GLY | 3015 | 39.856 | 21.896 | 54.243 | 1.00 | 44.03 |
| ATOM | 3154 | N | GLY | 3015 | 39.224 | 21.601 | 56.954 | 1.00 | 45.25 |
| ATOM | 3155 | CA | GLY | 3015 | 38.580 | 22.673 | 56.115 | 1.00 | 44.73 |
| ATOM | 3156 | N | HIS | 3016 | 39.205 | 24.055 | 54.213 | 1.00 | 42.94 |
| ATOM | 3157 | CA | HIS | 3016 | 39.869 | 24.278 | 52.940 | 1.00 | 42.02 |
| ATOM | 3158 | CB | HIS | 3016 | 39.485 | 25.616 | 52.314 | 1.00 | 42.47 |
| ATOM | 3159 | CG | HIS | 3016 | 39.822 | 25.705 | 50.857 | 1.00 | 43.64 |
| ATOM | 3160 | CD2 | HIS | 3016 | 39.085 | 26.107 | 49.792 | 1.00 | 43.67 |
| ATOM | 3161 | ND1 | HIS | 3016 | 41.055 | 25.343 | 50.354 | 1.00 | 44.09 |
| ATOM | 3162 | CE1 | HIS | 3016 | 41.063 | 25.519 | 49.044 | 1.00 | 44.44 |
| ATOM | 3163 | NE2 | HIS | 3016 | 39.880 | 25.982 | 48.677 | 1.00 | 44.26 |
| ATOM | 3164 | C | HIS | 3016 | 41.369 | 24.241 | 53.176 | 1.00 | 40.94 |
| ATOM | 3165 | O | HIS | 3016 | 41.903 | 24.895 | 54.072 | 1.00 | 40.43 |
| ATOM | 3166 | N | PHE | 3017 | 42.045 | 23.443 | 52.371 | 1.00 | 40.37 |
| ATOM | 3167 | CA | PHE | 3017 | 43.477 | 23.296 | 52.483 | 1.00 | 40.05 |
| ATOM | 3168 | CB | PHE | 3017 | 43.963 | 22.244 | 51.475 | 1.00 | 37.48 |
| ATOM | 3169 | CG | PHE | 3017 | 43.885 | 22.676 | 50.044 | 1.00 | 35.09 |
| ATOM | 3170 | CD1 | PHE | 3017 | 44.958 | 23.320 | 49.438 | 1.00 | 35.25 |
| ATOM | 3171 | CD2 | PHE | 3017 | 42.748 | 22.435 | 49.291 | 1.00 | 34.83 |
| ATOM | 3172 | CE1 | PHE | 3017 | 44.894 | 23.721 | 48.081 | 1.00 | 34.50 |
| ATOM | 3173 | CE2 | PHE | 3017 | 42.670 | 22.830 | 47.941 | 1.00 | 34.11 |
| ATOM | 3174 | CZ | PHE | 3017 | 43.746 | 23.472 | 47.341 | 1.00 | 34.04 |
| ATOM | 3175 | C | PHE | 3017 | 44.218 | 24.617 | 52.307 | 1.00 | 40.50 |
| ATOM | 3176 | O | PHE | 3017 | 45.309 | 24.783 | 52.853 | 1.00 | 40.69 |
| ATOM | 3177 | N | LYS | 3018 | 43.630 | 25.571 | 51.581 | 1.00 | 41.05 |
| ATOM | 3178 | CA | LYS | 3018 | 44.333 | 26.832 | 51.389 | 1.00 | 41.60 |
| ATOM | 3179 | CB | LYS | 3018 | 43.811 | 27.608 | 50.165 | 1.00 | 41.82 |
| ATOM | 3180 | CG | LYS | 3018 | 42.436 | 28.247 | 50.250 | 1.00 | 42.73 |
| ATOM | 3181 | CD | LYS | 3018 | 42.185 | 29.011 | 48.950 | 1.00 | 42.35 |
| ATOM | 3182 | CE | LYS | 3018 | 40.804 | 29.657 | 48.887 | 1.00 | 43.03 |
| ATOM | 3183 | NZ | LYS | 3018 | 39.735 | 28.707 | 48.434 | 1.00 | 43.02 |
| ATOM | 3184 | C | LYS | 3018 | 44.384 | 27.712 | 52.634 | 1.00 | 41.78 |
| ATOM | 3185 | O | LYS | 3018 | 45.353 | 28.450 | 52.822 | 1.00 | 42.20 |
| ATOM | 3186 | N | ASP | 3019 | 43.380 | 27.602 | 53.502 | 1.00 | 41.37 |
| ATOM | 3187 | CA | ASP | 3019 | 43.337 | 28.389 | 54.732 | 1.00 | 40.93 |
| ATOM | 3188 | CB | ASP | 3019 | 41.999 | 28.188 | 55.463 | 1.00 | 42.41 |
| ATOM | 3189 | CG | ASP | 3019 | 40.777 | 28.417 | 54.568 | 1.00 | 43.93 |
| ATOM | 3190 | OD1 | ASP | 3019 | 40.702 | 29.453 | 53.862 | 1.00 | 42.95 |
| ATOM | 3191 | OD2 | ASP | 3019 | 39.874 | 27.545 | 54.596 | 1.00 | 45.23 |
| ATOM | 3192 | C | ASP | 3019 | 44.470 | 28.026 | 55.704 | 1.00 | 39.79 |
| ATOM | 3193 | O | ASP | 3019 | 45.089 | 26.965 | 55.598 | 1.00 | 39.09 |
| ATOM | 3194 | N | PRO | 3020 | 44.752 | 28.921 | 56.667 | 1.00 | 38.78 |
| ATOM | 3195 | CD | PRO | 3020 | 44.222 | 30.292 | 56.741 | 1.00 | 38.65 |
| ATOM | 3196 | CA | PRO | 3020 | 45.793 | 28.725 | 57.677 | 1.00 | 37.71 |
| ATOM | 3197 | CB | PRO | 3020 | 45.897 | 30.099 | 58.344 | 1.00 | 37.25 |
| ATOM | 3198 | CG | PRO | 3020 | 45.382 | 31.032 | 57.323 | 1.00 | 38.14 |
| ATOM | 3199 | C | PRO | 3020 | 45.296 | 27.682 | 58.663 | 1.00 | 36.52 |
| ATOM | 3200 | O | PRO | 3020 | 44.095 | 27.471 | 58.794 | 1.00 | 36.09 |
| ATOM | 3201 | N | LYS | 3021 | 46.215 | 27.047 | 59.370 | 1.00 | 35.52 |
| ATOM | 3202 | CA | LYS | 3021 | 45.829 | 26.049 | 60.339 | 1.00 | 35.33 |
| ATOM | 3203 | CB | LYS | 3021 | 46.047 | 24.652 | 59.757 | 1.00 | 35.26 |
| ATOM | 3204 | CG | LYS | 3021 | 45.160 | 24.336 | 58.578 | 1.00 | 36.74 |
| ATOM | 3205 | CD | LYS | 3021 | 45.388 | 22.931 | 58.042 | 1.00 | 37.25 |
| ATOM | 3206 | CE | LYS | 3021 | 44.335 | 22.594 | 56.989 | 1.00 | 38.30 |
| ATOM | 3207 | NZ | LYS | 3021 | 44.605 | 21.291 | 56.299 | 1.00 | 39.33 |
| ATOM | 3208 | C | LYS | 3021 | 46.639 | 26.195 | 61.616 | 1.00 | 35.00 |
| ATOM | 3209 | O | LYS | 3021 | 47.706 | 26.807 | 61.620 | 1.00 | 34.55 |
| ATOM | 3210 | N | ARG | 3022 | 46.107 | 25.648 | 62.702 | 1.00 | 34.14 |
| ATOM | 3211 | CA | ARG | 3022 | 46.799 | 25.640 | 63.978 | 1.00 | 33.44 |
| ATOM | 3212 | CB | ARG | 3022 | 45.866 | 26.064 | 65.110 | 1.00 | 34.60 |
| ATOM | 3213 | CG | ARG | 3022 | 45.507 | 27.532 | 65.133 | 1.00 | 36.57 |
| ATOM | 3214 | CD | ARG | 3022 | 44.636 | 27.838 | 66.334 | 1.00 | 38.46 |
| ATOM | 3215 | NE | ARG | 3022 | 43.230 | 28.048 | 65.996 | 1.00 | 41.22 |
| ATOM | 3216 | CZ | ARG | 3022 | 42.712 | 29.217 | 65.617 | 1.00 | 43.64 |
| ATOM | 3217 | NH1 | ARG | 3022 | 43.481 | 30.299 | 65.522 | 1.00 | 43.54 |
| ATOM | 3218 | NH2 | ARG | 3022 | 41.414 | 29.310 | 65.343 | 1.00 | 44.87 |
| ATOM | 3219 | C | ARG | 3022 | 47.195 | 24.181 | 64.176 | 1.00 | 32.71 |
| ATOM | 3220 | O | ARG | 3022 | 46.418 | 23.279 | 63.841 | 1.00 | 32.40 |
| ATOM | 3221 | N | LEU | 3023 | 48.399 | 23.939 | 64.687 | 1.00 | 31.39 |
| ATOM | 3222 | CA | LEU | 3023 | 48.828 | 22.573 | 64.933 | 1.00 | 30.33 |
| ATOM | 3223 | CB | LEU | 3023 | 50.207 | 22.314 | 64.329 | 1.00 | 29.42 |
| ATOM | 3224 | CG | LEU | 3023 | 50.230 | 22.316 | 62.801 | 1.00 | 28.69 |
| ATOM | 3225 | CD1 | LEU | 3023 | 51.589 | 21.897 | 62.311 | 1.00 | 29.16 |
| ATOM | 3226 | CD2 | LEU | 3023 | 49.185 | 21.359 | 62.272 | 1.00 | 29.24 |
| ATOM | 3227 | C | LEU | 3023 | 48.836 | 22.317 | 66.428 | 1.00 | 30.43 |
| ATOM | 3228 | O | LEU | 3023 | 49.716 | 22.782 | 67.149 | 1.00 | 31.69 |
| ATOM | 3229 | N | TYR | 3024 | 47.825 | 21.580 | 66.874 | 1.00 | 29.88 |
| ATOM | 3230 | CA | TYR | 3024 | 47.628 | 21.228 | 68.274 | 1.00 | 30.17 |
| ATOM | 3231 | CB | TYR | 3024 | 46.134 | 20.993 | 68.493 | 1.00 | 29.97 |
| ATOM | 3232 | CG | TYR | 3024 | 45.723 | 20.633 | 69.893 | 1.00 | 30.23 |
| ATOM | 3233 | CD1 | TYR | 3024 | 45.670 | 19.303 | 70.307 | 1.00 | 29.34 |
| ATOM | 3234 | CE1 | TYR | 3024 | 45.246 | 18.969 | 71.591 | 1.00 | 29.30 |
| ATOM | 3235 | CD2 | TYR | 3024 | 45.347 | 21.627 | 70.805 | 1.00 | 30.96 |
| ATOM | 3236 | CE2 | TYR | 3024 | 44.924 | 21.302 | 72.085 | 1.00 | 30.56 |
| ATOM | 3237 | CZ | TYR | 3024 | 44.874 | 19.971 | 72.469 | 1.00 | 30.35 |
| ATOM | 3238 | OH | TYR | 3024 | 44.437 | 19.658 | 73.731 | 1.00 | 31.23 |
| ATOM | 3239 | C | TYR | 3024 | 48.415 | 19.966 | 68.587 | 1.00 | 30.47 |
| ATOM | 3240 | O | TYR | 3024 | 48.134 | 18.914 | 68.021 | 1.00 | 31.61 |
| ATOM | 3241 | N | CYS | 3025 | 49.394 | 20.062 | 69.481 | 1.00 | 30.30 |
| ATOM | 3242 | CA | CYS | 3025 | 50.227 | 18.914 | 69.831 | 1.00 | 30.43 |
| ATOM | 3243 | CB | CYS | 3025 | 51.566 | 19.398 | 70.387 | 1.00 | 29.84 |
| ATOM | 3244 | SG | CYS | 3025 | 52.790 | 18.105 | 70.665 | 1.00 | 30.02 |
| ATOM | 3245 | C | CYS | 3025 | 49.532 | 18.024 | 70.851 | 1.00 | 31.50 |
| ATOM | 3246 | O | CYS | 3025 | 49.011 | 18.507 | 71.852 | 1.00 | 32.82 |
| ATOM | 3247 | N | LYS | 3026 | 49.530 | 16.721 | 70.603 | 1.00 | 31.99 |
| ATOM | 3248 | CA | LYS | 3026 | 48.875 | 15.785 | 71.505 | 1.00 | 32.28 |
| ATOM | 3249 | CB | LYS | 3026 | 48.889 | 14.366 | 70.931 | 1.00 | 32.14 |
| ATOM | 3250 | CG | LYS | 3026 | 48.124 | 13.362 | 71.785 | 1.00 | 31.13 |
| ATOM | 3251 | CD | LYS | 3026 | 48.256 | 11.950 | 71.261 | 1.00 | 31.04 |
| ATOM | 3252 | CE | LYS | 3026 | 47.376 | 10.991 | 72.054 | 1.00 | 31.54 |
| ATOM | 3253 | NZ | LYS | 3026 | 47.681 | 9.542 | 71.762 | 1.00 | 31.20 |
| ATOM | 3254 | C | LYS | 3026 | 49.549 | 15.761 | 72.855 | 1.00 | 33.71 |
| ATOM | 3255 | O | LYS | 3026 | 48.928 | 15.421 | 73.864 | 1.00 | 33.88 |
| ATOM | 3256 | N | ASN | 3027 | 50.822 | 16.128 | 72.872 | 1.00 | 34.85 |
| ATOM | 3257 | CA | ASN | 3027 | 51.598 | 16.118 | 74.100 | 1.00 | 35.21 |
| ATOM | 3258 | CB | ASN | 3027 | 53.069 | 15.892 | 73.771 | 1.00 | 36.01 |
| ATOM | 3259 | CG | ASN | 3027 | 53.906 | 15.644 | 75.005 | 1.00 | 37.05 |
| ATOM | 3260 | OD1 | ASN | 3027 | 53.370 | 15.386 | 76.085 | 1.00 | 38.89 |
| ATOM | 3261 | ND2 | ASN | 3027 | 55.228 | 15.702 | 74.852 | 1.00 | 36.55 |
| ATOM | 3262 | C | ASN | 3027 | 51.448 | 17.388 | 74.923 | 1.00 | 35.82 |
| ATOM | 3263 | O | ASN | 3027 | 52.299 | 18.276 | 74.874 | 1.00 | 36.50 |
| ATOM | 3264 | N | GLY | 3028 | 50.360 | 17.470 | 75.682 | 1.00 | 36.10 |
| ATOM | 3265 | CA | GLY | 3028 | 50.130 | 18.632 | 76.519 | 1.00 | 35.14 |
| ATOM | 3266 | C | GLY | 3028 | 49.125 | 19.614 | 75.965 | 1.00 | 35.38 |
| ATOM | 3267 | O | GLY | 3028 | 48.673 | 20.494 | 76.683 | 1.00 | 35.74 |
| ATOM | 3268 | N | GLY | 3029 | 48.773 | 19.480 | 74.692 | 1.00 | 34.83 |
| ATOM | 3269 | CA | GLY | 3029 | 47.811 | 20.397 | 74.116 | 1.00 | 34.70 |
| ATOM | 3270 | C | GLY | 3029 | 48.368 | 21.775 | 73.785 | 1.00 | 34.41 |
| ATOM | 3271 | O | GLY | 3029 | 47.653 | 22.777 | 73.859 | 1.00 | 34.46 |
| ATOM | 3272 | N | PHE | 3030 | 49.645 | 21.834 | 73.418 | 1.00 | 33.79 |
| ATOM | 3273 | CA | PHE | 3030 | 50.267 | 23.101 | 73.050 | 1.00 | 32.58 |
| ATOM | 3274 | CB | PHE | 3030 | 51.731 | 23.158 | 73.499 | 1.00 | 31.51 |
| ATOM | 3275 | CG | PHE | 3030 | 51.916 | 23.120 | 74.979 | 1.00 | 29.52 |
| ATOM | 3276 | CD1 | PHE | 3030 | 52.106 | 21.909 | 75.639 | 1.00 | 29.11 |
| ATOM | 3277 | CD2 | PHE | 3030 | 51.876 | 24.296 | 75.721 | 1.00 | 28.85 |
| ATOM | 3278 | CE1 | PHE | 3030 | 52.255 | 21.869 | 77.029 | 1.00 | 28.80 |
| ATOM | 3279 | CE2 | PHE | 3030 | 52.020 | 24.269 | 77.097 | 1.00 | 28.58 |
| ATOM | 3280 | CZ | PHE | 3030 | 52.212 | 23.050 | 77.758 | 1.00 | 28.72 |
| ATOM | 3281 | C | PHE | 3030 | 50.232 | 23.293 | 71.549 | 1.00 | 32.66 |
| ATOM | 3282 | O | PHE | 3030 | 50.523 | 22.366 | 70.796 | 1.00 | 32.83 |
| ATOM | 3283 | N | PHE | 3031 | 49.876 | 24.499 | 71.123 | 1.00 | 32.38 |
| ATOM | 3284 | CA | PHE | 3031 | 49.846 | 24.842 | 69.704 | 1.00 | 32.69 |
| ATOM | 3285 | CB | PHE | 3031 | 48.925 | 26.041 | 69.481 | 1.00 | 31.36 |
| ATOM | 3286 | CG | PHE | 3031 | 47.475 | 25.721 | 69.625 | 1.00 | 30.70 |
| ATOM | 3287 | CD1 | PHE | 3031 | 46.814 | 24.996 | 68.645 | 1.00 | 30.66 |
| ATOM | 3288 | CD2 | PHE | 3031 | 46.763 | 26.156 | 70.735 | 1.00 | 30.44 |
| ATOM | 3289 | CE1 | PHE | 3031 | 45.462 | 24.711 | 68.763 | 1.00 | 31.15 |
| ATOM | 3290 | CE2 | PHE | 3031 | 45.412 | 25.880 | 70.868 | 1.00 | 30.36 |
| ATOM | 3291 | CZ | PHE | 3031 | 44.756 | 25.154 | 69.875 | 1.00 | 31.24 |
| ATOM | 3292 | C | PHE | 3031 | 51.268 | 25.203 | 69.244 | 1.00 | 32.94 |
| ATOM | 3293 | O | PHE | 3031 | 52.003 | 25.879 | 69.961 | 1.00 | 33.02 |
| ATOM | 3294 | N | LEU | 3032 | 51.659 | 24.752 | 68.059 | 1.00 | 33.24 |
| ATOM | 3295 | CA | LEU | 3032 | 52.982 | 25.071 | 67.548 | 1.00 | 33.97 |
| ATOM | 3296 | CB | LEU | 3032 | 53.242 | 24.312 | 66.247 | 1.00 | 33.70 |
| ATOM | 3297 | CG | LEU | 3032 | 54.668 | 24.432 | 65.694 | 1.00 | 33.92 |
| ATOM | 3298 | CD1 | LEU | 3032 | 55.669 | 23.894 | 66.717 | 1.00 | 32.95 |
| ATOM | 3299 | CD2 | LEU | 3032 | 54.781 | 23.669 | 64.381 | 1.00 | 32.92 |
| ATOM | 3300 | C | LEU | 3032 | 53.010 | 26.581 | 67.294 | 1.00 | 34.80 |
| ATOM | 3301 | O | LEU | 3032 | 52.130 | 27.110 | 66.617 | 1.00 | 33.89 |
| ATOM | 3302 | N | ARG | 3033 | 54.015 | 27.265 | 67.846 | 1.00 | 35.98 |
| ATOM | 3303 | CA | ARG | 3033 | 54.132 | 28.711 | 67.704 | 1.00 | 36.45 |
| ATOM | 3304 | CB | ARG | 3033 | 53.969 | 29.383 | 69.062 | 1.00 | 36.94 |
| ATOM | 3305 | CG | ARG | 3033 | 54.148 | 30.894 | 69.020 | 1.00 | 36.96 |
| ATOM | 3306 | CD | ARG | 3033 | 53.667 | 31.538 | 70.312 | 1.00 | 35.82 |
| ATOM | 3307 | NE | ARG | 3033 | 54.437 | 31.076 | 71.463 | 1.00 | 35.91 |
| ATOM | 3308 | CZ | ARG | 3033 | 54.244 | 31.493 | 72.712 | 1.00 | 35.56 |
| ATOM | 3309 | NH1 | ARG | 3033 | 53.297 | 32.389 | 72.979 | 1.00 | 34.92 |
| ATOM | 3310 | NH2 | ARG | 3033 | 54.997 | 31.016 | 73.695 | 1.00 | 33.93 |
| ATOM | 3311 | C | ARG | 3033 | 55.429 | 29.190 | 67.088 | 1.00 | 37.34 |
| ATOM | 3312 | O | ARG | 3033 | 56.517 | 28.802 | 67.517 | 1.00 | 37.17 |
| ATOM | 3313 | N | ILE | 3034 | 55.290 | 30.050 | 66.084 | 1.00 | 38.29 |
| ATOM | 3314 | CA | ILE | 3034 | 56.416 | 30.638 | 65.369 | 1.00 | 39.19 |
| ATOM | 3315 | CB | ILE | 3034 | 56.208 | 30.604 | 63.840 | 1.00 | 39.20 |
| ATOM | 3316 | CG2 | ILE | 3034 | 57.344 | 31.350 | 63.150 | 1.00 | 38.31 |
| ATOM | 3317 | CG1 | ILE | 3034 | 56.084 | 29.165 | 63.339 | 1.00 | 39.01 |
| ATOM | 3318 | CD1 | ILE | 3034 | 57.345 | 28.380 | 63.428 | 1.00 | 39.41 |
| ATOM | 3319 | C | ILE | 3034 | 56.474 | 32.109 | 65.746 | 1.00 | 40.28 |
| ATOM | 3320 | O | ILE | 3034 | 55.628 | 32.890 | 65.307 | 1.00 | 40.75 |
| ATOM | 3321 | N | HIS | 3035 | 57.462 | 32.489 | 66.547 | 1.00 | 41.64 |
| ATOM | 3322 | CA | HIS | 3035 | 57.621 | 33.883 | 66.959 | 1.00 | 43.21 |
| ATOM | 3323 | CB | HIS | 3035 | 58.529 | 33.961 | 68.180 | 1.00 | 43.78 |
| ATOM | 3324 | CG | HIS | 3035 | 57.870 | 33.550 | 69.455 | 1.00 | 44.79 |
| ATOM | 3325 | CD2 | HIS | 3035 | 57.900 | 32.382 | 70.140 | 1.00 | 45.08 |
| ATOM | 3326 | ND1 | HIS | 3035 | 57.078 | 34.407 | 70.191 | 1.00 | 44.77 |
| ATOM | 3327 | CE1 | HIS | 3035 | 56.655 | 33.786 | 71.278 | 1.00 | 45.08 |
| ATOM | 3328 | NE2 | HIS | 3035 | 57.139 | 32.556 | 71.271 | 1.00 | 45.86 |
| ATOM | 3329 | C | HIS | 3035 | 58.232 | 34.743 | 65.860 | 1.00 | 43.96 |
| ATOM | 3330 | O | HIS | 3035 | 59.040 | 34.268 | 65.062 | 1.00 | 43.75 |
| ATOM | 3331 | N | PRO | 3036 | 57.862 | 36.030 | 65.817 | 1.00 | 44.91 |
| ATOM | 3332 | CD | PRO | 3036 | 56.885 | 36.684 | 66.706 | 1.00 | 45.02 |
| ATOM | 3333 | CA | PRO | 3036 | 58.371 | 36.976 | 64.820 | 1.00 | 45.87 |
| ATOM | 3334 | CB | PRO | 3036 | 57.778 | 38.301 | 65.281 | 1.00 | 45.58 |
| ATOM | 3335 | CG | PRO | 3036 | 56.475 | 37.884 | 65.892 | 1.00 | 45.04 |
| ATOM | 3336 | C | PRO | 3036 | 59.901 | 37.013 | 64.789 | 1.00 | 46.93 |
| ATOM | 3337 | O | PRO | 3036 | 60.503 | 37.213 | 63.737 | 1.00 | 47.15 |
| ATOM | 3338 | N | ASP | 3037 | 60.526 | 36.807 | 65.944 | 1.00 | 48.32 |
| ATOM | 3339 | CA | ASP | 3037 | 61.985 | 36.829 | 66.030 | 1.00 | 49.80 |
| ATOM | 3340 | CB | ASP | 3037 | 62.420 | 37.251 | 67.428 | 1.00 | 51.06 |
| ATOM | 3341 | CG | ASP | 3037 | 61.964 | 36.280 | 68.487 | 1.00 | 52.96 |
| ATOM | 3342 | OD1 | ASP | 3037 | 61.386 | 35.235 | 68.108 | 1.00 | 53.57 |
| ATOM | 3343 | OD2 | ASP | 3037 | 62.181 | 36.557 | 69.689 | 1.00 | 53.50 |
| ATOM | 3344 | C | ASP | 3037 | 62.675 | 35.505 | 65.680 | 1.00 | 50.28 |
| ATOM | 3345 | O | ASP | 3037 | 63.896 | 35.387 | 65.815 | 1.00 | 50.87 |
| ATOM | 3346 | N | GLY | 3038 | 61.907 | 34.507 | 65.248 | 1.00 | 50.09 |
| ATOM | 3347 | CA | GLY | 3038 | 62.508 | 33.235 | 64.884 | 1.00 | 49.42 |
| ATOM | 3348 | C | GLY | 3038 | 62.446 | 32.149 | 65.942 | 1.00 | 49.27 |
| ATOM | 3349 | O | GLY | 3038 | 62.916 | 31.036 | 65.708 | 1.00 | 49.47 |
| ATOM | 3350 | N | ARG | 3039 | 61.871 | 32.455 | 67.101 | 1.00 | 48.78 |
| ATOM | 3351 | CA | ARG | 3039 | 61.761 | 31.468 | 68.170 | 1.00 | 48.40 |
| ATOM | 3352 | CB | ARG | 3039 | 61.658 | 32.162 | 69.532 | 1.00 | 49.72 |
| ATOM | 3353 | CG | ARG | 3039 | 62.964 | 32.773 | 70.010 | 1.00 | 51.91 |
| ATOM | 3354 | CD | ARG | 3039 | 62.872 | 33.317 | 71.434 | 1.00 | 53.71 |
| ATOM | 3355 | NE | ARG | 3039 | 61.972 | 34.467 | 71.553 | 1.00 | 55.03 |
| ATOM | 3356 | CZ | ARG | 3039 | 60.698 | 34.400 | 71.935 | 1.00 | 55.24 |
| ATOM | 3357 | NH1 | ARG | 3039 | 59.974 | 35.514 | 72.005 | 1.00 | 55.06 |
| ATOM | 3358 | NH2 | ARG | 3039 | 60.152 | 33.228 | 72.257 | 1.00 | 54.61 |
| ATOM | 3359 | C | ARG | 3039 | 60.549 | 30.567 | 67.962 | 1.00 | 47.46 |
| ATOM | 3360 | O | ARG | 3039 | 59.475 | 31.045 | 67.594 | 1.00 | 47.82 |
| ATOM | 3361 | N | VAL | 3040 | 60.728 | 29.268 | 68.194 | 1.00 | 45.77 |
| ATOM | 3362 | CA | VAL | 3040 | 59.645 | 28.299 | 68.043 | 1.00 | 44.63 |
| ATOM | 3363 | CB | VAL | 3040 | 59.968 | 27.230 | 66.967 | 1.00 | 44.49 |
| ATOM | 3364 | CG1 | VAL | 3040 | 58.797 | 26.257 | 66.833 | 1.00 | 43.73 |
| ATOM | 3365 | CG2 | VAL | 3040 | 60.250 | 27.889 | 65.637 | 1.00 | 44.68 |
| ATOM | 3366 | C | VAL | 3040 | 59.373 | 27.561 | 69.352 | 1.00 | 44.13 |
| ATOM | 3367 | O | VAL | 3040 | 60.286 | 27.016 | 69.975 | 1.00 | 43.72 |
| ATOM | 3368 | N | ASP | 3041 | 58.111 | 27.543 | 69.762 | 1.00 | 43.53 |
| ATOM | 3369 | CA | ASP | 3041 | 57.710 | 26.852 | 70.985 | 1.00 | 43.09 |
| ATOM | 3370 | CB | ASP | 3041 | 57.999 | 27.713 | 72.219 | 1.00 | 43.33 |
| ATOM | 3371 | CG | ASP | 3041 | 57.118 | 28.948 | 72.293 | 1.00 | 43.69 |
| ATOM | 3372 | OD1 | ASP | 3041 | 57.136 | 29.620 | 73.346 | 1.00 | 44.37 |
| ATOM | 3373 | OD2 | ASP | 3041 | 56.411 | 29.249 | 71.306 | 1.00 | 44.09 |
| ATOM | 3374 | C | ASP | 3041 | 56.218 | 26.553 | 70.918 | 1.00 | 42.82 |
| ATOM | 3375 | O | ASP | 3041 | 55.613 | 26.633 | 69.847 | 1.00 | 42.19 |
| ATOM | 3376 | N | GLY | 3042 | 55.628 | 26.227 | 72.066 | 1.00 | 42.21 |
| ATOM | 3377 | CA | GLY | 3042 | 54.207 | 25.934 | 72.105 | 1.00 | 42.24 |
| ATOM | 3378 | C | GLY | 3042 | 53.452 | 26.747 | 73.141 | 1.00 | 42.40 |
| ATOM | 3379 | O | GLY | 3042 | 54.025 | 27.195 | 74.132 | 1.00 | 42.44 |
| ATOM | 3380 | N | VAL | 3043 | 52.158 | 26.932 | 72.907 | 1.00 | 42.44 |
| ATOM | 3381 | CA | VAL | 3043 | 51.295 | 27.694 | 73.802 | 1.00 | 42.96 |
| ATOM | 3382 | CB | VAL | 3043 | 51.045 | 29.127 | 73.301 | 1.00 | 43.37 |
| ATOM | 3383 | CG1 | VAL | 3043 | 51.277 | 30.131 | 74.421 | 1.00 | 43.42 |
| ATOM | 3384 | CG2 | VAL | 3043 | 51.883 | 29.401 | 72.089 | 1.00 | 44.01 |
| ATOM | 3385 | C | VAL | 3043 | 49.937 | 27.044 | 73.765 | 1.00 | 43.12 |
| ATOM | 3386 | O | VAL | 3043 | 49.553 | 26.483 | 72.743 | 1.00 | 43.37 |
| ATOM | 3387 | N | ARG | 3044 | 49.197 | 27.163 | 74.859 | 1.00 | 43.20 |
| ATOM | 3388 | CA | ARG | 3044 | 47.866 | 26.594 | 74.939 | 1.00 | 43.44 |
| ATOM | 3389 | CB | ARG | 3044 | 47.578 | 26.157 | 76.369 | 1.00 | 42.93 |
| ATOM | 3390 | CG | ARG | 3044 | 48.401 | 24.989 | 76.870 | 1.00 | 43.13 |
| ATOM | 3391 | CD | ARG | 3044 | 47.491 | 24.144 | 77.729 | 1.00 | 42.95 |
| ATOM | 3392 | NE | ARG | 3044 | 48.096 | 22.920 | 78.225 | 1.00 | 43.60 |
| ATOM | 3393 | CZ | ARG | 3044 | 48.867 | 22.850 | 79.299 | 1.00 | 43.94 |
| ATOM | 3394 | NH1 | ARG | 3044 | 49.136 | 23.952 | 79.989 | 1.00 | 44.31 |
| ATOM | 3395 | NH2 | ARG | 3044 | 49.339 | 21.671 | 79.700 | 1.00 | 43.40 |
| ATOM | 3396 | C | ARG | 3044 | 46.785 | 27.573 | 74.498 | 1.00 | 44.18 |
| ATOM | 3397 | O | ARG | 3044 | 45.740 | 27.156 | 74.021 | 1.00 | 44.98 |
| ATOM | 3398 | N | GLU | 3045 | 47.025 | 28.872 | 74.655 | 1.00 | 45.38 |
| ATOM | 3399 | CA | GLU | 3045 | 46.006 | 29.850 | 74.282 | 1.00 | 46.95 |
| ATOM | 3400 | CB | GLU | 3045 | 46.369 | 31.257 | 74.781 | 1.00 | 48.46 |
| ATOM | 3401 | CG | GLU | 3045 | 45.293 | 32.310 | 74.475 | 1.00 | 51.18 |
| ATOM | 3402 | CD | GLU | 3045 | 43.933 | 32.026 | 75.142 | 1.00 | 53.25 |
| ATOM | 3403 | OE1 | GLU | 3045 | 43.865 | 32.063 | 76.393 | 1.00 | 53.75 |
| ATOM | 3404 | OE2 | GLU | 3045 | 42.932 | 31.774 | 74.416 | 1.00 | 53.34 |
| ATOM | 3405 | C | GLU | 3045 | 45.726 | 29.871 | 72.780 | 1.00 | 46.70 |
| ATOM | 3406 | O | GLU | 3045 | 46.562 | 30.274 | 71.973 | 1.00 | 45.91 |
| ATOM | 3407 | N | LYS | 3046 | 44.526 | 29.430 | 72.423 | 1.00 | 46.85 |
| ATOM | 3408 | CA | LYS | 3046 | 44.116 | 29.361 | 71.037 | 1.00 | 47.15 |
| ATOM | 3409 | CB | LYS | 3046 | 42.742 | 28.707 | 70.916 | 1.00 | 48.04 |
| ATOM | 3410 | CG | LYS | 3046 | 42.327 | 28.499 | 69.467 | 1.00 | 50.38 |
| ATOM | 3411 | CD | LYS | 3046 | 40.927 | 27.927 | 69.339 | 1.00 | 52.53 |
| ATOM | 3412 | CE | LYS | 3046 | 40.557 | 27.726 | 67.874 | 1.00 | 53.17 |
| ATOM | 3413 | NZ | LYS | 3046 | 39.180 | 27.181 | 67.721 | 1.00 | 54.74 |
| ATOM | 3414 | C | LYS | 3046 | 44.088 | 30.705 | 70.333 | 1.00 | 47.10 |
| ATOM | 3415 | O | LYS | 3046 | 44.144 | 30.760 | 69.105 | 1.00 | 47.26 |
| ATOM | 3416 | N | SER | 3047 | 44.011 | 31.789 | 71.100 | 1.00 | 47.49 |
| ATOM | 3417 | CA | SER | 3047 | 43.959 | 33.128 | 70.513 | 1.00 | 47.14 |
| ATOM | 3418 | CB | SER | 3047 | 43.127 | 34.056 | 71.397 | 1.00 | 46.20 |
| ATOM | 3419 | OG | SER | 3047 | 43.605 | 34.051 | 72.727 | 1.00 | 46.24 |
| ATOM | 3420 | C | SER | 3047 | 45.325 | 33.760 | 70.226 | 1.00 | 47.37 |
| ATOM | 3421 | O | SER | 3047 | 45.398 | 34.851 | 69.652 | 1.00 | 47.61 |
| ATOM | 3422 | N | ASP | 3048 | 46.403 | 33.082 | 70.617 | 1.00 | 46.88 |
| ATOM | 3423 | CA | ASP | 3048 | 47.742 | 33.594 | 70.351 | 1.00 | 46.89 |
| ATOM | 3424 | CB | ASP | 3048 | 48.787 | 32.564 | 70.796 | 1.00 | 46.86 |
| ATOM | 3425 | CG | ASP | 3048 | 50.219 | 33.082 | 70.687 | 1.00 | 47.31 |
| ATOM | 3426 | OD1 | ASP | 3048 | 51.066 | 32.681 | 71.519 | 1.00 | 46.33 |
| ATOM | 3427 | OD2 | ASP | 3048 | 50.502 | 33.876 | 69.765 | 1.00 | 47.57 |
| ATOM | 3428 | C | ASP | 3048 | 47.831 | 33.859 | 68.836 | 1.00 | 46.86 |
| ATOM | 3429 | O | ASP | 3048 | 47.414 | 33.032 | 68.025 | 1.00 | 47.10 |
| ATOM | 3430 | N | PRO | 3049 | 48.358 | 35.028 | 68.438 | 1.00 | 46.48 |
| ATOM | 3431 | CD | PRO | 3049 | 48.786 | 36.168 | 69.275 | 1.00 | 46.50 |
| ATOM | 3432 | CA | PRO | 3049 | 48.464 | 35.347 | 67.007 | 1.00 | 45.56 |
| ATOM | 3433 | CB | PRO | 3049 | 48.636 | 36.861 | 67.009 | 1.00 | 46.07 |
| ATOM | 3434 | CG | PRO | 3049 | 49.464 | 37.082 | 68.266 | 1.00 | 47.16 |
| ATOM | 3435 | C | PRO | 3049 | 49.579 | 34.649 | 66.234 | 1.00 | 44.55 |
| ATOM | 3436 | O | PRO | 3049 | 49.539 | 34.594 | 65.006 | 1.00 | 44.85 |
| ATOM | 3437 | N | HIS | 3050 | 50.563 | 34.104 | 66.940 | 1.00 | 43.58 |
| ATOM | 3438 | CA | HIS | 3050 | 51.686 | 33.450 | 66.276 | 1.00 | 42.38 |
| ATOM | 3439 | CB | HIS | 3050 | 52.983 | 33.747 | 67.015 | 1.00 | 43.14 |
| ATOM | 3440 | CG | HIS | 3050 | 53.173 | 35.194 | 67.329 | 1.00 | 44.43 |
| ATOM | 3441 | CD2 | HIS | 3050 | 53.439 | 35.825 | 68.497 | 1.00 | 44.84 |
| ATOM | 3442 | ND1 | HIS | 3050 | 53.095 | 36.179 | 66.369 | 1.00 | 45.12 |
| ATOM | 3443 | CE1 | HIS | 3050 | 53.305 | 37.355 | 66.931 | 1.00 | 46.07 |
| ATOM | 3444 | NE2 | HIS | 3050 | 53.516 | 37.168 | 68.222 | 1.00 | 46.13 |
| ATOM | 3445 | C | HIS | 3050 | 51.576 | 31.952 | 66.115 | 1.00 | 41.17 |
| ATOM | 3446 | O | HIS | 3050 | 52.597 | 31.281 | 65.960 | 1.00 | 41.43 |
| ATOM | 3447 | N | ILE | 3051 | 50.362 | 31.415 | 66.158 | 1.00 | 39.64 |
| ATOM | 3448 | CA | ILE | 3051 | 50.198 | 29.979 | 65.995 | 1.00 | 38.07 |
| ATOM | 3449 | CB | ILE | 3051 | 49.519 | 29.327 | 67.232 | 1.00 | 36.79 |
| ATOM | 3450 | CG2 | ILE | 3051 | 50.350 | 29.601 | 68.463 | 1.00 | 35.71 |
| ATOM | 3451 | CG1 | ILE | 3051 | 48.108 | 29.876 | 67.437 | 1.00 | 36.28 |
| ATOM | 3452 | CD1 | ILE | 3051 | 47.340 | 29.199 | 68.556 | 1.00 | 35.71 |
| ATOM | 3453 | C | ILE | 3051 | 49.428 | 29.669 | 64.717 | 1.00 | 38.09 |
| ATOM | 3454 | O | ILE | 3051 | 49.241 | 28.509 | 64.366 | 1.00 | 38.24 |
| ATOM | 3455 | N | LYS | 3052 | 48.985 | 30.716 | 64.026 | 1.00 | 37.53 |
| ATOM | 3456 | CA | LYS | 3052 | 48.287 | 30.539 | 62.763 | 1.00 | 37.74 |
| ATOM | 3457 | CB | LYS | 3052 | 47.504 | 31.801 | 62.394 | 1.00 | 38.81 |
| ATOM | 3458 | CG | LYS | 3052 | 46.326 | 32.052 | 63.344 | 1.00 | 40.63 |
| ATOM | 3459 | CD | LYS | 3052 | 45.191 | 32.817 | 62.674 | 1.00 | 41.29 |
| ATOM | 3460 | CE | LYS | 3052 | 43.930 | 32.777 | 63.537 | 1.00 | 42.64 |
| ATOM | 3461 | NZ | LYS | 3052 | 42.700 | 33.226 | 62.798 | 1.00 | 43.00 |
| ATOM | 3462 | C | LYS | 3052 | 49.379 | 30.248 | 61.744 | 1.00 | 36.94 |
| ATOM | 3463 | O | LYS | 3052 | 50.222 | 31.097 | 61.460 | 1.00 | 36.70 |
| ATOM | 3464 | N | LEU | 3053 | 49.368 | 29.031 | 61.216 | 1.00 | 35.75 |
| ATOM | 3465 | CA | LEU | 3053 | 50.385 | 28.593 | 60.278 | 1.00 | 34.26 |
| ATOM | 3466 | CB | LEU | 3053 | 51.042 | 27.320 | 60.813 | 1.00 | 32.88 |
| ATOM | 3467 | CG | LEU | 3053 | 51.320 | 27.279 | 62.320 | 1.00 | 32.14 |
| ATOM | 3468 | CD1 | LEU | 3053 | 51.970 | 25.957 | 62.679 | 1.00 | 32.01 |
| ATOM | 3469 | CD2 | LEU | 3053 | 52.209 | 28.439 | 62.729 | 1.00 | 31.66 |
| ATOM | 3470 | C | LEU | 3053 | 49.842 | 28.327 | 58.880 | 1.00 | 34.38 |
| ATOM | 3471 | O | LEU | 3053 | 48.668 | 28.024 | 58.694 | 1.00 | 34.66 |
| ATOM | 3472 | N | GLN | 3054 | 50.713 | 28.441 | 57.891 | 1.00 | 34.09 |
| ATOM | 3473 | CA | GLN | 3054 | 50.315 | 28.188 | 56.526 | 1.00 | 33.75 |
| ATOM | 3474 | CB | GLN | 3054 | 50.600 | 29.408 | 55.662 | 1.00 | 34.84 |
| ATOM | 3475 | CG | GLN | 3054 | 50.064 | 29.266 | 54.258 | 1.00 | 36.90 |
| ATOM | 3476 | CD | GLN | 3054 | 48.560 | 29.067 | 54.239 | 1.00 | 37.93 |
| ATOM | 3477 | OE1 | GLN | 3054 | 47.790 | 30.028 | 54.245 | 1.00 | 37.57 |
| ATOM | 3478 | NE2 | GLN | 3054 | 48.134 | 27.807 | 54.236 | 1.00 | 40.03 |
| ATOM | 3479 | C | GLN | 3054 | 51.115 | 26.994 | 56.031 | 1.00 | 33.08 |
| ATOM | 3480 | O | GLN | 3054 | 52.310 | 27.104 | 55.800 | 1.00 | 32.80 |
| ATOM | 3481 | N | LEU | 3055 | 50.458 | 25.844 | 55.898 | 1.00 | 32.84 |
| ATOM | 3482 | CA | LEU | 3055 | 51.130 | 24.639 | 55.436 | 1.00 | 31.57 |
| ATOM | 3483 | CB | LEU | 3055 | 50.527 | 23.389 | 56.069 | 1.00 | 32.17 |
| ATOM | 3484 | CG | LEU | 3055 | 50.354 | 23.353 | 57.592 | 1.00 | 33.60 |
| ATOM | 3485 | CD1 | LEU | 3055 | 49.949 | 21.946 | 58.000 | 1.00 | 34.08 |
| ATOM | 3486 | CD2 | LEU | 3055 | 51.626 | 23.747 | 58.295 | 1.00 | 33.96 |
| ATOM | 3487 | C | LEU | 3055 | 50.979 | 24.582 | 53.937 | 1.00 | 31.32 |
| ATOM | 3488 | O | LEU | 3055 | 49.872 | 24.571 | 53.410 | 1.00 | 32.02 |
| ATOM | 3489 | N | GLN | 3056 | 52.110 | 24.563 | 53.253 | 1.00 | 31.15 |
| ATOM | 3490 | CA | GLN | 3056 | 52.128 | 24.539 | 51.798 | 1.00 | 30.82 |
| ATOM | 3491 | CB | GLN | 3056 | 52.769 | 25.820 | 51.270 | 1.00 | 29.37 |
| ATOM | 3492 | CG | GLN | 3056 | 52.975 | 25.840 | 49.788 | 1.00 | 30.22 |
| ATOM | 3493 | CD | GLN | 3056 | 51.667 | 25.849 | 49.013 | 1.00 | 30.34 |
| ATOM | 3494 | OE1 | GLN | 3056 | 50.859 | 26.764 | 49.155 | 1.00 | 30.39 |
| ATOM | 3495 | NE2 | GLN | 3056 | 51.464 | 24.837 | 48.177 | 1.00 | 28.72 |
| ATOM | 3496 | C | GLN | 3056 | 52.898 | 23.341 | 51.263 | 1.00 | 31.21 |
| ATOM | 3497 | O | GLN | 3056 | 54.059 | 23.125 | 51.612 | 1.00 | 31.76 |
| ATOM | 3498 | N | ALA | 3057 | 52.251 | 22.563 | 50.411 | 1.00 | 31.42 |
| ATOM | 3499 | CA | ALA | 3057 | 52.905 | 21.410 | 49.828 | 1.00 | 32.68 |
| ATOM | 3500 | CB | ALA | 3057 | 51.876 | 20.496 | 49.194 | 1.00 | 32.40 |
| ATOM | 3501 | C | ALA | 3057 | 53.898 | 21.893 | 48.773 | 1.00 | 33.28 |
| ATOM | 3502 | O | ALA | 3057 | 53.577 | 22.769 | 47.972 | 1.00 | 33.33 |
| ATOM | 3503 | N | GLU | 3058 | 55.106 | 21.337 | 48.790 | 1.00 | 33.43 |
| ATOM | 3504 | CA | GLU | 3058 | 56.122 | 21.707 | 47.818 | 1.00 | 34.14 |
| ATOM | 3505 | CB | GLU | 3058 | 57.494 | 21.793 | 48.490 | 1.00 | 34.57 |
| ATOM | 3506 | CG | GLU | 3058 | 58.554 | 22.591 | 47.704 | 1.00 | 35.75 |
| ATOM | 3507 | CD | GLU | 3058 | 58.123 | 24.035 | 47.373 | 1.00 | 36.06 |
| ATOM | 3508 | OE1 | GLU | 3058 | 57.646 | 24.753 | 48.278 | 1.00 | 35.34 |
| ATOM | 3509 | OE2 | GLU | 3058 | 58.277 | 24.455 | 46.202 | 1.00 | 36.63 |
| ATOM | 3510 | C | GLU | 3058 | 56.087 | 20.622 | 46.748 | 1.00 | 34.36 |
| ATOM | 3511 | O | GLU | 3058 | 56.423 | 20.849 | 45.592 | 1.00 | 34.43 |
| ATOM | 3512 | N | GLU | 3059 | 55.661 | 19.438 | 47.168 | 1.00 | 34.56 |
| ATOM | 3513 | CA | GLU | 3059 | 55.508 | 18.283 | 46.295 | 1.00 | 34.61 |
| ATOM | 3514 | CB | GLU | 3059 | 56.872 | 17.683 | 45.909 | 1.00 | 35.10 |
| ATOM | 3515 | CG | GLU | 3059 | 57.648 | 17.011 | 47.016 | 1.00 | 37.26 |
| ATOM | 3516 | CD | GLU | 3059 | 59.074 | 16.676 | 46.595 | 1.00 | 38.48 |
| ATOM | 3517 | OE1 | GLU | 3059 | 59.856 | 17.628 | 46.351 | 1.00 | 38.84 |
| ATOM | 3518 | OE2 | GLU | 3059 | 59.412 | 15.470 | 46.501 | 1.00 | 38.95 |
| ATOM | 3519 | C | GLU | 3059 | 54.660 | 17.301 | 47.089 | 1.00 | 33.62 |
| ATOM | 3520 | O | GLU | 3059 | 54.314 | 17.576 | 48.227 | 1.00 | 33.74 |
| ATOM | 3521 | N | ARG | 3060 | 54.321 | 16.171 | 46.491 | 1.00 | 32.97 |
| ATOM | 3522 | CA | ARG | 3060 | 53.491 | 15.180 | 47.151 | 1.00 | 32.79 |
| ATOM | 3523 | CB | ARG | 3060 | 53.379 | 13.951 | 46.240 | 1.00 | 32.92 |
| ATOM | 3524 | CG | ARG | 3060 | 52.365 | 12.936 | 46.696 | 1.00 | 34.05 |
| ATOM | 3525 | CD | ARG | 3060 | 52.415 | 11.684 | 45.846 | 1.00 | 35.21 |
| ATOM | 3526 | NE | ARG | 3060 | 51.771 | 10.570 | 46.535 | 1.00 | 37.38 |
| ATOM | 3527 | CZ | ARG | 3060 | 51.790 | 9.316 | 46.105 | 1.00 | 38.21 |
| ATOM | 3528 | NH1 | ARG | 3060 | 52.419 | 9.014 | 44.980 | 1.00 | 38.75 |
| ATOM | 3529 | NH2 | ARG | 3060 | 51.199 | 8.361 | 46.814 | 1.00 | 39.26 |
| ATOM | 3530 | C | ARG | 3060 | 53.988 | 14.777 | 48.550 | 1.00 | 31.96 |
| ATOM | 3531 | O | ARG | 3060 | 55.104 | 14.291 | 48.708 | 1.00 | 31.33 |
| ATOM | 3532 | N | GLY | 3061 | 53.152 | 15.001 | 49.560 | 1.00 | 31.18 |
| ATOM | 3533 | CA | GLY | 3061 | 53.499 | 14.630 | 50.917 | 1.00 | 30.00 |
| ATOM | 3534 | C | GLY | 3061 | 54.552 | 15.465 | 51.612 | 1.00 | 29.89 |
| ATOM | 3535 | O | GLY | 3061 | 54.901 | 15.183 | 52.768 | 1.00 | 29.97 |
| ATOM | 3536 | N | VAL | 3062 | 55.064 | 16.485 | 50.927 | 1.00 | 28.74 |
| ATOM | 3537 | CA | VAL | 3062 | 56.086 | 17.344 | 51.513 | 1.00 | 28.56 |
| ATOM | 3538 | CB | VAL | 3062 | 57.378 | 17.355 | 50.662 | 1.00 | 29.32 |
| ATOM | 3539 | CG1 | VAL | 3062 | 58.445 | 18.189 | 51.361 | 1.00 | 28.02 |
| ATOM | 3540 | CG2 | VAL | 3062 | 57.868 | 15.928 | 50.431 | 1.00 | 28.21 |
| ATOM | 3541 | C | VAL | 3062 | 55.584 | 18.774 | 51.650 | 1.00 | 28.15 |
| ATOM | 3542 | O | VAL | 3062 | 55.147 | 19.377 | 50.675 | 1.00 | 28.13 |
| ATOM | 3543 | N | VAL | 3063 | 55.660 | 19.315 | 52.862 | 1.00 | 27.50 |
| ATOM | 3544 | CA | VAL | 3063 | 55.189 | 20.669 | 53.123 | 1.00 | 27.18 |
| ATOM | 3545 | CB | VAL | 3063 | 53.949 | 20.683 | 54.058 | 1.00 | 26.17 |
| ATOM | 3546 | CG1 | VAL | 3063 | 52.833 | 19.830 | 53.484 | 1.00 | 26.29 |
| ATOM | 3547 | CG2 | VAL | 3063 | 54.342 | 20.201 | 55.438 | 1.00 | 24.81 |
| ATOM | 3548 | C | VAL | 3063 | 56.202 | 21.572 | 53.806 | 1.00 | 28.09 |
| ATOM | 3549 | O | VAL | 3063 | 57.218 | 21.120 | 54.350 | 1.00 | 28.46 |
| ATOM | 3550 | N | SER | 3064 | 55.895 | 22.863 | 53.777 | 1.00 | 28.28 |
| ATOM | 3551 | CA | SER | 3064 | 56.693 | 23.881 | 54.441 | 1.00 | 28.12 |
| ATOM | 3552 | CB | SER | 3064 | 57.116 | 24.979 | 53.460 | 1.00 | 27.42 |
| ATOM | 3553 | OG | SER | 3064 | 56.046 | 25.873 | 53.198 | 1.00 | 26.90 |
| ATOM | 3554 | C | SER | 3064 | 55.678 | 24.445 | 55.443 | 1.00 | 28.57 |
| ATOM | 3555 | O | SER | 3064 | 54.472 | 24.457 | 55.169 | 1.00 | 28.91 |
| ATOM | 3556 | N | ILE | 3065 | 56.153 | 24.898 | 56.592 | 1.00 | 28.30 |
| ATOM | 3557 | CA | ILE | 3065 | 55.271 | 25.436 | 57.619 | 1.00 | 28.82 |
| ATOM | 3558 | CB | ILE | 3065 | 55.374 | 24.586 | 58.928 | 1.00 | 27.57 |
| ATOM | 3559 | CG2 | ILE | 3065 | 54.543 | 25.206 | 60.034 | 1.00 | 25.09 |
| ATOM | 3560 | CG1 | ILE | 3065 | 54.950 | 23.141 | 58.630 | 1.00 | 26.69 |
| ATOM | 3561 | CD1 | ILE | 3065 | 55.331 | 22.113 | 59.713 | 1.00 | 24.99 |
| ATOM | 3562 | C | ILE | 3065 | 55.663 | 26.885 | 57.901 | 1.00 | 30.19 |
| ATOM | 3563 | O | ILE | 3065 | 56.713 | 27.165 | 58.477 | 1.00 | 30.93 |
| ATOM | 3564 | N | LYS | 3066 | 54.811 | 27.806 | 57.488 | 1.00 | 31.05 |
| ATOM | 3565 | CA | LYS | 3066 | 55.083 | 29.210 | 57.680 | 1.00 | 32.25 |
| ATOM | 3566 | CB | LYS | 3066 | 54.947 | 29.929 | 56.333 | 1.00 | 33.41 |
| ATOM | 3567 | CG | LYS | 3066 | 54.965 | 31.448 | 56.419 | 1.00 | 35.49 |
| ATOM | 3568 | CD | LYS | 3066 | 54.891 | 32.097 | 55.042 | 1.00 | 36.65 |
| ATOM | 3569 | CE | LYS | 3066 | 54.678 | 33.603 | 55.189 | 1.00 | 38.51 |
| ATOM | 3570 | NZ | LYS | 3066 | 54.835 | 34.366 | 53.915 | 1.00 | 39.94 |
| ATOM | 3571 | C | LYS | 3066 | 54.193 | 29.874 | 58.731 | 1.00 | 32.84 |
| ATOM | 3572 | O | LYS | 3066 | 52.964 | 29.754 | 58.692 | 1.00 | 31.87 |
| ATOM | 3573 | N | GLY | 3067 | 54.829 | 30.560 | 59.683 | 1.00 | 33.82 |
| ATOM | 3574 | CA | GLY | 3067 | 54.090 | 31.275 | 60.711 | 1.00 | 34.94 |
| ATOM | 3575 | C | GLY | 3067 | 53.614 | 32.563 | 60.063 | 1.00 | 36.00 |
| ATOM | 3576 | O | GLY | 3067 | 54.424 | 33.424 | 59.756 | 1.00 | 36.30 |
| ATOM | 3577 | N | VAL | 3068 | 52.309 | 32.689 | 59.841 | 1.00 | 36.83 |
| ATOM | 3578 | CA | VAL | 3068 | 51.748 | 33.861 | 59.183 | 1.00 | 37.77 |
| ATOM | 3579 | CB | VAL | 3068 | 50.211 | 33.871 | 59.242 | 1.00 | 37.69 |
| ATOM | 3580 | CG1 | VAL | 3068 | 49.683 | 35.127 | 58.552 | 1.00 | 36.95 |
| ATOM | 3581 | CG2 | VAL | 3068 | 49.652 | 32.624 | 58.574 | 1.00 | 37.29 |
| ATOM | 3582 | C | VAL | 3068 | 52.226 | 35.208 | 59.694 | 1.00 | 38.84 |
| ATOM | 3583 | O | VAL | 3068 | 52.812 | 35.979 | 58.936 | 1.00 | 39.91 |
| ATOM | 3584 | N | SER | 3069 | 51.975 | 35.511 | 60.964 | 1.00 | 39.30 |
| ATOM | 3585 | CA | SER | 3069 | 52.396 | 36.805 | 61.475 | 1.00 | 39.93 |
| ATOM | 3586 | CB | SER | 3069 | 51.687 | 37.146 | 62.808 | 1.00 | 39.67 |
| ATOM | 3587 | OG | SER | 3069 | 52.335 | 36.611 | 63.942 | 1.00 | 40.20 |
| ATOM | 3588 | C | SER | 3069 | 53.917 | 36.939 | 61.599 | 1.00 | 39.81 |
| ATOM | 3589 | O | SER | 3069 | 54.452 | 38.024 | 61.418 | 1.00 | 40.29 |
| ATOM | 3590 | N | ALA | 3070 | 54.622 | 35.855 | 61.885 | 1.00 | 39.95 |
| ATOM | 3591 | CA | ALA | 3070 | 56.077 | 35.955 | 61.993 | 1.00 | 40.08 |
| ATOM | 3592 | CB | ALA | 3070 | 56.637 | 34.772 | 62.756 | 1.00 | 39.80 |
| ATOM | 3593 | C | ALA | 3070 | 56.739 | 36.040 | 60.616 | 1.00 | 40.33 |
| ATOM | 3594 | O | ALA | 3070 | 57.894 | 36.452 | 60.499 | 1.00 | 40.24 |
| ATOM | 3595 | N | ASN | 3071 | 55.998 | 35.657 | 59.579 | 1.00 | 40.38 |
| ATOM | 3596 | CA | ASN | 3071 | 56.498 | 35.660 | 58.205 | 1.00 | 40.07 |
| ATOM | 3597 | CB | ASN | 3071 | 56.754 | 37.089 | 57.710 | 1.00 | 40.54 |
| ATOM | 3598 | CG | ASN | 3071 | 56.686 | 37.208 | 56.179 | 1.00 | 41.80 |
| ATOM | 3599 | OD1 | ASN | 3071 | 56.789 | 36.214 | 55.456 | 1.00 | 42.23 |
| ATOM | 3600 | ND2 | ASN | 3071 | 56.516 | 38.431 | 55.688 | 1.00 | 41.81 |
| ATOM | 3601 | C | ASN | 3071 | 57.795 | 34.859 | 58.102 | 1.00 | 40.04 |
| ATOM | 3602 | O | ASN | 3071 | 58.704 | 35.230 | 57.364 | 1.00 | 40.69 |
| ATOM | 3603 | N | ARG | 3072 | 57.882 | 33.761 | 58.847 | 1.00 | 39.57 |
| ATOM | 3604 | CA | ARG | 3072 | 59.068 | 32.915 | 58.828 | 1.00 | 38.51 |
| ATOM | 3605 | CB | ARG | 3072 | 59.833 | 33.057 | 60.134 | 1.00 | 38.85 |
| ATOM | 3606 | CG | ARG | 3072 | 60.284 | 34.458 | 60.451 | 1.00 | 38.92 |
| ATOM | 3607 | CD | ARG | 3072 | 60.985 | 34.474 | 61.797 | 1.00 | 39.72 |
| ATOM | 3608 | NE | ARG | 3072 | 61.722 | 35.711 | 62.020 | 1.00 | 39.77 |
| ATOM | 3609 | CZ | ARG | 3072 | 63.038 | 35.835 | 61.874 | 1.00 | 39.87 |
| ATOM | 3610 | NH1 | ARG | 3072 | 63.779 | 34.795 | 61.502 | 1.00 | 39.94 |
| ATOM | 3611 | NH2 | ARG | 3072 | 63.614 | 37.003 | 62.109 | 1.00 | 39.73 |
| ATOM | 3612 | C | ARG | 3072 | 58.674 | 31.453 | 58.641 | 1.00 | 38.21 |
| ATOM | 3613 | O | ARG | 3072 | 57.569 | 31.050 | 59.001 | 1.00 | 38.04 |
| ATOM | 3614 | N | TYR | 3073 | 59.586 | 30.663 | 58.084 | 1.00 | 37.44 |
| ATOM | 3615 | CA | TYR | 3073 | 59.335 | 29.244 | 57.852 | 1.00 | 36.55 |
| ATOM | 3616 | CB | TYR | 3073 | 59.843 | 28.815 | 56.480 | 1.00 | 35.14 |
| ATOM | 3617 | CG | TYR | 3073 | 59.228 | 29.577 | 55.344 | 1.00 | 34.29 |
| ATOM | 3618 | CD1 | TYR | 3073 | 59.626 | 30.884 | 55.057 | 1.00 | 34.30 |
| ATOM | 3619 | CE1 | TYR | 3073 | 59.042 | 31.594 | 54.020 | 1.00 | 34.12 |
| ATOM | 3620 | CD2 | TYR | 3073 | 58.231 | 29.000 | 54.562 | 1.00 | 33.18 |
| ATOM | 3621 | CE2 | TYR | 3073 | 57.643 | 29.693 | 53.527 | 1.00 | 33.26 |
| ATOM | 3622 | CZ | TYR | 3073 | 58.048 | 30.986 | 53.258 | 1.00 | 34.05 |
| ATOM | 3623 | OH | TYR | 3073 | 57.451 | 31.667 | 52.234 | 1.00 | 34.61 |
| ATOM | 3624 | C | TYR | 3073 | 60.012 | 28.380 | 58.891 | 1.00 | 36.96 |
| ATOM | 3625 | O | TYR | 3073 | 61.155 | 28.633 | 59.269 | 1.00 | 37.99 |
| ATOM | 3626 | N | LEU | 3074 | 59.315 | 27.348 | 59.346 | 1.00 | 36.71 |
| ATOM | 3627 | CA | LEU | 3074 | 59.890 | 26.456 | 60.338 | 1.00 | 36.55 |
| ATOM | 3628 | CB | LEU | 3074 | 58.833 | 25.501 | 60.887 | 1.00 | 36.70 |
| ATOM | 3629 | CG | LEU | 3074 | 59.327 | 24.591 | 62.014 | 1.00 | 36.08 |
| ATOM | 3630 | CD1 | LEU | 3074 | 59.342 | 25.402 | 63.308 | 1.00 | 36.24 |
| ATOM | 3631 | CD2 | LEU | 3074 | 58.425 | 23.350 | 62.153 | 1.00 | 35.50 |
| ATOM | 3632 | C | LEU | 3074 | 61.014 | 25.654 | 59.692 | 1.00 | 36.84 |
| ATOM | 3633 | O | LEU | 3074 | 60.900 | 25.200 | 58.552 | 1.00 | 35.51 |
| ATOM | 3634 | N | ALA | 3075 | 62.101 | 25.484 | 60.436 | 1.00 | 38.12 |
| ATOM | 3635 | CA | ALA | 3075 | 63.259 | 24.744 | 59.951 | 1.00 | 39.17 |
| ATOM | 3636 | CB | ALA | 3075 | 64.300 | 25.710 | 59.379 | 1.00 | 38.85 |
| ATOM | 3637 | C | ALA | 3075 | 63.870 | 23.959 | 61.087 | 1.00 | 39.54 |
| ATOM | 3638 | O | ALA | 3075 | 63.848 | 24.404 | 62.227 | 1.00 | 40.34 |
| ATOM | 3639 | N | MET | 3076 | 64.400 | 22.784 | 60.771 | 1.00 | 40.43 |
| ATOM | 3640 | CA | MET | 3076 | 65.053 | 21.948 | 61.767 | 1.00 | 41.46 |
| ATOM | 3641 | CB | MET | 3076 | 64.456 | 20.540 | 61.798 | 1.00 | 40.39 |
| ATOM | 3642 | CG | MET | 3076 | 65.075 | 19.688 | 62.893 | 1.00 | 40.68 |
| ATOM | 3643 | SD | MET | 3076 | 64.362 | 18.062 | 63.123 | 1.00 | 40.51 |
| ATOM | 3644 | CE | MET | 3076 | 65.209 | 17.227 | 61.954 | 1.00 | 41.15 |
| ATOM | 3645 | C | MET | 3076 | 66.529 | 21.872 | 61.397 | 1.00 | 42.83 |
| ATOM | 3646 | O | MET | 3076 | 66.878 | 21.581 | 60.252 | 1.00 | 42.41 |
| ATOM | 3647 | N | LYS | 3077 | 67.395 | 22.123 | 62.372 | 1.00 | 44.33 |
| ATOM | 3648 | CA | LYS | 3077 | 68.828 | 22.122 | 62.126 | 1.00 | 45.26 |
| ATOM | 3649 | CB | LYS | 3077 | 69.486 | 23.097 | 63.091 | 1.00 | 45.85 |
| ATOM | 3650 | CG | LYS | 3077 | 68.786 | 24.451 | 63.140 | 1.00 | 46.46 |
| ATOM | 3651 | CD | LYS | 3077 | 68.690 | 25.084 | 61.760 | 1.00 | 46.81 |
| ATOM | 3652 | CE | LYS | 3077 | 70.071 | 25.269 | 61.139 | 1.00 | 46.97 |
| ATOM | 3653 | NZ | LYS | 3077 | 69.998 | 25.859 | 59.775 | 1.00 | 46.20 |
| ATOM | 3654 | C | LYS | 3077 | 69.487 | 20.749 | 62.222 | 1.00 | 45.55 |
| ATOM | 3655 | O | LYS | 3077 | 68.845 | 19.767 | 62.584 | 1.00 | 45.67 |
| ATOM | 3656 | N | GLU | 3078 | 70.774 | 20.698 | 61.889 | 1.00 | 45.60 |
| ATOM | 3657 | CA | GLU | 3078 | 71.547 | 19.460 | 61.915 | 1.00 | 46.22 |
| ATOM | 3658 | CB | GLU | 3078 | 72.958 | 19.703 | 61.368 | 1.00 | 46.38 |
| ATOM | 3659 | CG | GLU | 3078 | 73.839 | 20.534 | 62.288 | 1.00 | 46.71 |
| ATOM | 3660 | CD | GLU | 3078 | 73.426 | 21.988 | 62.350 | 1.00 | 47.56 |
| ATOM | 3661 | OE1 | GLU | 3078 | 73.551 | 22.599 | 63.433 | 1.00 | 47.08 |
| ATOM | 3662 | OE2 | GLU | 3078 | 72.990 | 22.524 | 61.307 | 1.00 | 48.41 |
| ATOM | 3663 | C | GLU | 3078 | 71.649 | 18.839 | 63.308 | 1.00 | 46.26 |
| ATOM | 3664 | O | GLU | 3078 | 71.840 | 17.631 | 63.437 | 1.00 | 46.11 |
| ATOM | 3665 | N | ASP | 3079 | 71.539 | 19.663 | 64.346 | 1.00 | 46.55 |
| ATOM | 3666 | CA | ASP | 3079 | 71.613 | 19.164 | 65.720 | 1.00 | 46.41 |
| ATOM | 3667 | CB | ASP | 3079 | 72.210 | 20.229 | 66.651 | 1.00 | 46.70 |
| ATOM | 3668 | CG | ASP | 3079 | 71.386 | 21.500 | 66.688 | 1.00 | 47.53 |
| ATOM | 3669 | OD1 | ASP | 3079 | 71.717 | 22.407 | 67.482 | 1.00 | 48.21 |
| ATOM | 3670 | OD2 | ASP | 3079 | 70.406 | 21.593 | 65.918 | 1.00 | 48.26 |
| ATOM | 3671 | C | ASP | 3079 | 70.223 | 18.755 | 66.223 | 1.00 | 45.67 |
| ATOM | 3672 | O | ASP | 3079 | 70.095 | 18.102 | 67.262 | 1.00 | 45.13 |
| ATOM | 3673 | N | GLY | 3080 | 69.188 | 19.149 | 65.480 | 1.00 | 44.71 |
| ATOM | 3674 | CA | GLY | 3080 | 67.830 | 18.800 | 65.853 | 1.00 | 43.08 |
| ATOM | 3675 | C | GLY | 3080 | 67.004 | 19.931 | 66.425 | 1.00 | 42.34 |
| ATOM | 3676 | O | GLY | 3080 | 65.816 | 19.762 | 66.673 | 1.00 | 41.81 |
| ATOM | 3677 | N | ARG | 3081 | 67.614 | 21.093 | 66.638 | 1.00 | 42.18 |
| ATOM | 3678 | CA | ARG | 3081 | 66.862 | 22.209 | 67.199 | 1.00 | 40.95 |
| ATOM | 3679 | CB | ARG | 3081 | 67.798 | 23.257 | 67.828 | 1.00 | 41.24 |
| ATOM | 3680 | CG | ARG | 3081 | 68.640 | 24.040 | 66.865 | 1.00 | 40.94 |
| ATOM | 3681 | CD | ARG | 3081 | 69.351 | 25.211 | 67.542 | 1.00 | 40.92 |
| ATOM | 3682 | NE | ARG | 3081 | 69.923 | 26.095 | 66.531 | 1.00 | 41.22 |
| ATOM | 3683 | CZ | ARG | 3081 | 70.849 | 25.718 | 65.651 | 1.00 | 41.42 |
| ATOM | 3684 | NH1 | ARG | 3081 | 71.325 | 24.478 | 65.667 | 1.00 | 40.19 |
| ATOM | 3685 | NH2 | ARG | 3081 | 71.266 | 26.562 | 64.718 | 1.00 | 41.75 |
| ATOM | 3686 | C | ARG | 3081 | 65.972 | 22.840 | 66.144 | 1.00 | 39.61 |
| ATOM | 3687 | O | ARG | 3081 | 66.208 | 22.691 | 64.944 | 1.00 | 38.72 |
| ATOM | 3688 | N | LEU | 3082 | 64.932 | 23.527 | 66.602 | 1.00 | 38.75 |
| ATOM | 3689 | CA | LEU | 3082 | 63.977 | 24.171 | 65.706 | 1.00 | 37.74 |
| ATOM | 3690 | CB | LEU | 3082 | 62.549 | 23.777 | 66.079 | 1.00 | 35.64 |
| ATOM | 3691 | CG | LEU | 3082 | 62.113 | 22.318 | 66.035 | 1.00 | 33.29 |
| ATOM | 3692 | CD1 | LEU | 3082 | 60.630 | 22.251 | 66.353 | 1.00 | 32.47 |
| ATOM | 3693 | CD2 | LEU | 3082 | 62.377 | 21.729 | 64.666 | 1.00 | 32.11 |
| ATOM | 3694 | C | LEU | 3082 | 64.072 | 25.682 | 65.745 | 1.00 | 38.14 |
| ATOM | 3695 | O | LEU | 3082 | 64.391 | 26.269 | 66.776 | 1.00 | 38.98 |
| ATOM | 3696 | N | LEU | 3083 | 63.780 | 26.313 | 64.619 | 1.00 | 38.47 |
| ATOM | 3697 | CA | LEU | 3083 | 63.808 | 27.763 | 64.545 | 1.00 | 39.01 |
| ATOM | 3698 | CB | LEU | 3083 | 65.255 | 28.271 | 64.500 | 1.00 | 38.86 |
| ATOM | 3699 | CG | LEU | 3083 | 66.229 | 27.792 | 63.422 | 1.00 | 38.86 |
| ATOM | 3700 | CD1 | LEU | 3083 | 65.852 | 28.379 | 62.068 | 1.00 | 39.17 |
| ATOM | 3701 | CD2 | LEU | 3083 | 67.631 | 28.239 | 63.797 | 1.00 | 38.83 |
| ATOM | 3702 | C | LEU | 3083 | 63.043 | 28.185 | 63.311 | 1.00 | 39.17 |
| ATOM | 3703 | O | LEU | 3083 | 62.773 | 27.359 | 62.448 | 1.00 | 39.05 |
| ATOM | 3704 | N | ALA | 3084 | 62.679 | 29.460 | 63.231 | 1.00 | 39.49 |
| ATOM | 3705 | CA | ALA | 3084 | 61.940 | 29.946 | 62.075 | 1.00 | 40.69 |
| ATOM | 3706 | CB | ALA | 3084 | 60.661 | 30.671 | 62.517 | 1.00 | 40.89 |
| ATOM | 3707 | C | ALA | 3084 | 62.808 | 30.855 | 61.210 | 1.00 | 41.31 |
| ATOM | 3708 | O | ALA | 3084 | 63.125 | 31.991 | 61.572 | 1.00 | 41.89 |
| ATOM | 3709 | N | SER | 3085 | 63.182 | 30.320 | 60.059 | 1.00 | 41.13 |
| ATOM | 3710 | CA | SER | 3085 | 64.016 | 30.994 | 59.081 | 1.00 | 41.28 |
| ATOM | 3711 | CB | SER | 3085 | 64.605 | 29.914 | 58.170 | 1.00 | 41.02 |
| ATOM | 3712 | OG | SER | 3085 | 65.154 | 30.443 | 56.989 | 1.00 | 42.75 |
| ATOM | 3713 | C | SER | 3085 | 63.215 | 32.012 | 58.267 | 1.00 | 41.63 |
| ATOM | 3714 | O | SER | 3085 | 62.039 | 31.799 | 57.978 | 1.00 | 41.39 |
| ATOM | 3715 | N | LYS | 3086 | 63.851 | 33.119 | 57.893 | 1.00 | 42.23 |
| ATOM | 3716 | CA | LYS | 3086 | 63.185 | 34.148 | 57.101 | 1.00 | 42.36 |
| ATOM | 3717 | CB | LYS | 3086 | 63.926 | 35.487 | 57.227 | 1.00 | 43.35 |
| ATOM | 3718 | CG | LYS | 3086 | 63.208 | 36.676 | 56.590 | 1.00 | 44.87 |
| ATOM | 3719 | CD | LYS | 3086 | 61.900 | 37.057 | 57.325 | 1.00 | 46.41 |
| ATOM | 3720 | CE | LYS | 3086 | 61.100 | 38.119 | 56.533 | 1.00 | 47.44 |
| ATOM | 3721 | NZ | LYS | 3086 | 59.894 | 38.663 | 57.241 | 1.00 | 47.24 |
| ATOM | 3722 | C | LYS | 3086 | 63.109 | 33.715 | 55.640 | 1.00 | 41.96 |
| ATOM | 3723 | O | LYS | 3086 | 62.189 | 34.092 | 54.923 | 1.00 | 41.53 |
| ATOM | 3724 | N | SER | 3087 | 64.068 | 32.911 | 55.201 | 1.00 | 41.87 |
| ATOM | 3725 | CA | SER | 3087 | 64.065 | 32.436 | 53.823 | 1.00 | 42.60 |
| ATOM | 3726 | CB | SER | 3087 | 65.348 | 32.872 | 53.100 | 1.00 | 43.24 |
| ATOM | 3727 | OG | SER | 3087 | 66.484 | 32.166 | 53.582 | 1.00 | 44.56 |
| ATOM | 3728 | C | SER | 3087 | 63.936 | 30.912 | 53.784 | 1.00 | 42.46 |
| ATOM | 3729 | O | SER | 3087 | 64.232 | 30.231 | 54.763 | 1.00 | 42.59 |
| ATOM | 3730 | N | VAL | 3088 | 63.505 | 30.381 | 52.646 | 1.00 | 42.17 |
| ATOM | 3731 | CA | VAL | 3088 | 63.318 | 28.945 | 52.498 | 1.00 | 41.98 |
| ATOM | 3732 | CB | VAL | 3088 | 62.270 | 28.639 | 51.419 | 1.00 | 41.66 |
| ATOM | 3733 | CG1 | VAL | 3088 | 62.195 | 27.144 | 51.186 | 1.00 | 42.36 |
| ATOM | 3734 | CG2 | VAL | 3088 | 60.914 | 29.186 | 51.840 | 1.00 | 41.06 |
| ATOM | 3735 | C | VAL | 3088 | 64.578 | 28.185 | 52.139 | 1.00 | 42.05 |
| ATOM | 3736 | O | VAL | 3088 | 65.212 | 28.480 | 51.138 | 1.00 | 42.63 |
| ATOM | 3737 | N | THR | 3089 | 64.937 | 27.200 | 52.953 | 1.00 | 42.64 |
| ATOM | 3738 | CA | THR | 3089 | 66.116 | 26.381 | 52.677 | 1.00 | 43.00 |
| ATOM | 3739 | CB | THR | 3089 | 67.221 | 26.554 | 53.723 | 1.00 | 43.23 |
| ATOM | 3740 | OG1 | THR | 3089 | 66.889 | 25.793 | 54.891 | 1.00 | 44.39 |
| ATOM | 3741 | CG2 | THR | 3089 | 67.379 | 28.015 | 54.094 | 1.00 | 43.24 |
| ATOM | 3742 | C | THR | 3089 | 65.670 | 24.931 | 52.710 | 1.00 | 43.33 |
| ATOM | 3743 | O | THR | 3089 | 64.529 | 24.640 | 53.068 | 1.00 | 43.41 |
| ATOM | 3744 | N | ASP | 3090 | 66.560 | 24.014 | 52.346 | 1.00 | 43.54 |
| ATOM | 3745 | CA | ASP | 3090 | 66.186 | 22.607 | 52.337 | 1.00 | 43.60 |
| ATOM | 3746 | CB | ASP | 3090 | 67.266 | 21.763 | 51.658 | 1.00 | 44.79 |
| ATOM | 3747 | CG | ASP | 3090 | 68.630 | 21.940 | 52.286 | 1.00 | 47.12 |
| ATOM | 3748 | OD1 | ASP | 3090 | 68.765 | 22.784 | 53.205 | 1.00 | 47.64 |
| ATOM | 3749 | OD2 | ASP | 3090 | 69.571 | 21.231 | 51.846 | 1.00 | 48.18 |
| ATOM | 3750 | C | ASP | 3090 | 65.867 | 22.047 | 53.720 | 1.00 | 42.60 |
| ATOM | 3751 | O | ASP | 3090 | 65.414 | 20.917 | 53.838 | 1.00 | 43.28 |
| ATOM | 3752 | N | GLU | 3091 | 66.091 | 22.836 | 54.764 | 1.00 | 41.23 |
| ATOM | 3753 | CA | GLU | 3091 | 65.790 | 22.394 | 56.122 | 1.00 | 40.12 |
| ATOM | 3754 | CB | GLU | 3091 | 66.802 | 22.967 | 57.115 | 1.00 | 39.75 |
| ATOM | 3755 | CG | GLU | 3091 | 68.236 | 22.519 | 56.911 | 1.00 | 39.82 |
| ATOM | 3756 | CD | GLU | 3091 | 69.180 | 23.181 | 57.904 | 1.00 | 39.95 |
| ATOM | 3757 | OE1 | GLU | 3091 | 69.192 | 24.431 | 57.967 | 1.00 | 39.67 |
| ATOM | 3758 | OE2 | GLU | 3091 | 69.906 | 22.459 | 58.622 | 1.00 | 39.35 |
| ATOM | 3759 | C | GLU | 3091 | 64.384 | 22.841 | 56.543 | 1.00 | 39.14 |
| ATOM | 3760 | O | GLU | 3091 | 64.003 | 22.710 | 57.707 | 1.00 | 38.75 |
| ATOM | 3761 | N | CYS | 3092 | 63.620 | 23.362 | 55.592 | 1.00 | 37.51 |
| ATOM | 3762 | CA | CYS | 3092 | 62.278 | 23.844 | 55.874 | 1.00 | 35.78 |
| ATOM | 3763 | CB | CYS | 3092 | 62.100 | 25.236 | 55.279 | 1.00 | 35.93 |
| ATOM | 3764 | SG | CYS | 3092 | 63.209 | 26.450 | 55.977 | 1.00 | 35.58 |
| ATOM | 3765 | C | CYS | 3092 | 61.177 | 22.940 | 55.353 | 1.00 | 34.53 |
| ATOM | 3766 | O | CYS | 3092 | 60.006 | 23.304 | 55.404 | 1.00 | 33.70 |
| ATOM | 3767 | N | PHE | 3093 | 61.547 | 21.767 | 54.856 | 1.00 | 33.56 |
| ATOM | 3768 | CA | PHE | 3093 | 60.561 | 20.851 | 54.318 | 1.00 | 32.68 |
| ATOM | 3769 | CB | PHE | 3093 | 60.899 | 20.547 | 52.862 | 1.00 | 33.28 |
| ATOM | 3770 | CG | PHE | 3093 | 60.855 | 21.762 | 51.988 | 1.00 | 34.25 |
| ATOM | 3771 | CD1 | PHE | 3093 | 59.637 | 22.328 | 51.631 | 1.00 | 34.91 |
| ATOM | 3772 | CD2 | PHE | 3093 | 62.030 | 22.402 | 51.605 | 1.00 | 34.79 |
| ATOM | 3773 | CE1 | PHE | 3093 | 59.585 | 23.515 | 50.914 | 1.00 | 35.42 |
| ATOM | 3774 | CE2 | PHE | 3093 | 61.995 | 23.592 | 50.889 | 1.00 | 34.42 |
| ATOM | 3775 | CZ | PHE | 3093 | 60.771 | 24.151 | 50.544 | 1.00 | 35.85 |
| ATOM | 3776 | C | PHE | 3093 | 60.407 | 19.590 | 55.139 | 1.00 | 32.10 |
| ATOM | 3777 | O | PHE | 3093 | 61.386 | 19.004 | 55.607 | 1.00 | 32.02 |
| ATOM | 3778 | N | PHE | 3094 | 59.154 | 19.185 | 55.313 | 1.00 | 30.72 |
| ATOM | 3779 | CA | PHE | 3094 | 58.823 | 18.021 | 56.115 | 1.00 | 29.42 |
| ATOM | 3780 | CB | PHE | 3094 | 58.234 | 18.492 | 57.435 | 1.00 | 27.90 |
| ATOM | 3781 | CG | PHE | 3094 | 59.118 | 19.444 | 58.176 | 1.00 | 27.67 |
| ATOM | 3782 | CD1 | PHE | 3094 | 60.093 | 18.972 | 59.051 | 1.00 | 27.64 |
| ATOM | 3783 | CD2 | PHE | 3094 | 59.009 | 20.813 | 57.975 | 1.00 | 26.67 |
| ATOM | 3784 | CE1 | PHE | 3094 | 60.938 | 19.851 | 59.710 | 1.00 | 27.53 |
| ATOM | 3785 | CE2 | PHE | 3094 | 59.852 | 21.697 | 58.630 | 1.00 | 26.63 |
| ATOM | 3786 | CZ | PHE | 3094 | 60.817 | 21.218 | 59.498 | 1.00 | 26.89 |
| ATOM | 3787 | C | PHE | 3094 | 57.825 | 17.109 | 55.440 | 1.00 | 29.46 |
| ATOM | 3788 | O | PHE | 3094 | 56.974 | 17.559 | 54.675 | 1.00 | 30.00 |
| ATOM | 3789 | N | PHE | 3095 | 57.940 | 15.819 | 55.713 | 1.00 | 28.94 |
| ATOM | 3790 | CA | PHE | 3095 | 56.992 | 14.865 | 55.181 | 1.00 | 29.43 |
| ATOM | 3791 | CB | PHE | 3095 | 57.567 | 13.453 | 55.197 | 1.00 | 29.78 |
| ATOM | 3792 | CG | PHE | 3095 | 58.794 | 13.289 | 54.357 | 1.00 | 30.21 |
| ATOM | 3793 | CD1 | PHE | 3095 | 60.046 | 13.166 | 54.949 | 1.00 | 30.68 |
| ATOM | 3794 | CD2 | PHE | 3095 | 58.699 | 13.245 | 52.972 | 1.00 | 29.76 |
| ATOM | 3795 | CE1 | PHE | 3095 | 61.192 | 12.997 | 54.166 | 1.00 | 30.75 |
| ATOM | 3796 | CE2 | PHE | 3095 | 59.833 | 13.078 | 52.186 | 1.00 | 30.75 |
| ATOM | 3797 | CZ | PHE | 3095 | 61.082 | 12.953 | 52.788 | 1.00 | 30.46 |
| ATOM | 3798 | C | PHE | 3095 | 55.795 | 14.931 | 56.123 | 1.00 | 29.70 |
| ATOM | 3799 | O | PHE | 3095 | 55.922 | 14.669 | 57.332 | 1.00 | 29.21 |
| ATOM | 3800 | N | GLU | 3096 | 54.643 | 15.316 | 55.584 | 1.00 | 29.61 |
| ATOM | 3801 | CA | GLU | 3096 | 53.435 | 15.393 | 56.391 | 1.00 | 29.21 |
| ATOM | 3802 | CB | GLU | 3096 | 52.543 | 16.549 | 55.947 | 1.00 | 27.70 |
| ATOM | 3803 | CG | GLU | 3096 | 51.171 | 16.557 | 56.617 | 1.00 | 25.47 |
| ATOM | 3804 | CD | GLU | 3096 | 50.332 | 17.762 | 56.210 | 1.00 | 25.09 |
| ATOM | 3805 | OE1 | GLU | 3096 | 50.100 | 17.935 | 54.990 | 1.00 | 23.17 |
| ATOM | 3806 | OE2 | GLU | 3096 | 49.910 | 18.531 | 57.106 | 1.00 | 22.61 |
| ATOM | 3807 | C | GLU | 3096 | 52.691 | 14.086 | 56.225 | 1.00 | 29.12 |
| ATOM | 3808 | O | GLU | 3096 | 52.327 | 13.704 | 55.116 | 1.00 | 29.01 |
| ATOM | 3809 | N | ARG | 3097 | 52.460 | 13.401 | 57.332 | 1.00 | 29.53 |
| ATOM | 3810 | CA | ARG | 3097 | 51.767 | 12.138 | 57.263 | 1.00 | 30.40 |
| ATOM | 3811 | CB | ARG | 3097 | 52.773 | 11.023 | 57.542 | 1.00 | 32.38 |
| ATOM | 3812 | CG | ARG | 3097 | 52.349 | 9.642 | 57.123 | 1.00 | 36.15 |
| ATOM | 3813 | CD | ARG | 3097 | 53.436 | 8.649 | 57.532 | 1.00 | 39.45 |
| ATOM | 3814 | NE | ARG | 3097 | 53.000 | 7.250 | 57.522 | 1.00 | 42.19 |
| ATOM | 3815 | CZ | ARG | 3097 | 52.501 | 6.616 | 56.461 | 1.00 | 44.12 |
| ATOM | 3816 | NH1 | ARG | 3097 | 52.360 | 7.254 | 55.300 | 1.00 | 45.30 |
| ATOM | 3817 | NH2 | ARG | 3097 | 52.160 | 5.332 | 56.556 | 1.00 | 45.03 |
| ATOM | 3818 | C | ARG | 3097 | 50.581 | 12.072 | 58.226 | 1.00 | 29.29 |
| ATOM | 3819 | O | ARG | 3097 | 50.678 | 12.445 | 59.395 | 1.00 | 29.75 |
| ATOM | 3820 | N | LEU | 3098 | 49.438 | 11.645 | 57.704 | 1.00 | 28.52 |
| ATOM | 3821 | CA | LEU | 3098 | 48.249 | 11.470 | 58.524 | 1.00 | 27.07 |
| ATOM | 3822 | CB | LEU | 3098 | 46.967 | 11.688 | 57.715 | 1.00 | 26.03 |
| ATOM | 3823 | CG | LEU | 3098 | 45.647 | 11.196 | 58.338 | 1.00 | 26.58 |
| ATOM | 3824 | CD1 | LEU | 3098 | 45.526 | 11.621 | 59.807 | 1.00 | 26.51 |
| ATOM | 3825 | CD2 | LEU | 3098 | 44.480 | 11.746 | 57.523 | 1.00 | 26.38 |
| ATOM | 3826 | C | LEU | 3098 | 48.365 | 10.026 | 58.988 | 1.00 | 27.25 |
| ATOM | 3827 | O | LEU | 3098 | 48.089 | 9.082 | 58.238 | 1.00 | 27.80 |
| ATOM | 3828 | N | GLU | 3099 | 48.814 | 9.866 | 60.225 | 1.00 | 26.66 |
| ATOM | 3829 | CA | GLU | 3099 | 49.016 | 8.552 | 60.804 | 1.00 | 26.80 |
| ATOM | 3830 | CB | GLU | 3099 | 49.770 | 8.691 | 62.126 | 1.00 | 27.56 |
| ATOM | 3831 | CG | GLU | 3099 | 51.117 | 9.388 | 61.989 | 1.00 | 28.08 |
| ATOM | 3832 | CD | GLU | 3099 | 52.049 | 8.668 | 61.029 | 1.00 | 29.96 |
| ATOM | 3833 | OE1 | GLU | 3099 | 53.158 | 9.198 | 60.758 | 1.00 | 30.54 |
| ATOM | 3834 | OE2 | GLU | 3099 | 51.681 | 7.570 | 60.543 | 1.00 | 29.96 |
| ATOM | 3835 | C | GLU | 3099 | 47.718 | 7.791 | 61.016 | 1.00 | 27.03 |
| ATOM | 3836 | O | GLU | 3099 | 46.633 | 8.377 | 61.030 | 1.00 | 26.19 |
| ATOM | 3837 | N | SER | 3100 | 47.841 | 6.478 | 61.194 | 1.00 | 26.88 |
| ATOM | 3838 | CA | SER | 3100 | 46.683 | 5.630 | 61.398 | 1.00 | 26.68 |
| ATOM | 3839 | CB | SER | 3100 | 47.111 | 4.164 | 61.376 | 1.00 | 27.44 |
| ATOM | 3840 | OG | SER | 3100 | 48.012 | 3.873 | 62.427 | 1.00 | 29.67 |
| ATOM | 3841 | C | SER | 3100 | 45.939 | 5.957 | 62.696 | 1.00 | 26.58 |
| ATOM | 3842 | O | SER | 3100 | 44.794 | 5.542 | 62.887 | 1.00 | 26.50 |
| ATOM | 3843 | N | ASN | 3101 | 46.581 | 6.703 | 63.590 | 1.00 | 26.11 |
| ATOM | 3844 | CA | ASN | 3101 | 45.939 | 7.091 | 64.842 | 1.00 | 25.39 |
| ATOM | 3845 | CB | ASN | 3101 | 46.969 | 7.192 | 65.979 | 1.00 | 25.02 |
| ATOM | 3846 | CG | ASN | 3101 | 48.100 | 8.172 | 65.675 | 1.00 | 26.77 |
| ATOM | 3847 | OD1 | ASN | 3101 | 47.944 | 9.099 | 64.872 | 1.00 | 28.88 |
| ATOM | 3848 | ND2 | ASN | 3101 | 49.247 | 7.979 | 66.332 | 1.00 | 25.52 |
| ATOM | 3849 | C | ASN | 3101 | 45.207 | 8.428 | 64.685 | 1.00 | 25.05 |
| ATOM | 3850 | O | ASN | 3101 | 44.718 | 8.992 | 65.659 | 1.00 | 24.75 |
| ATOM | 3851 | N | ASN | 3102 | 45.147 | 8.920 | 63.451 | 1.00 | 24.62 |
| ATOM | 3852 | CA | ASN | 3102 | 44.482 | 10.179 | 63.110 | 1.00 | 24.62 |
| ATOM | 3853 | CB | ASN | 3102 | 43.053 | 10.185 | 63.626 | 1.00 | 24.47 |
| ATOM | 3854 | CG | ASN | 3102 | 42.192 | 9.189 | 62.898 | 1.00 | 25.74 |
| ATOM | 3855 | OD1 | ASN | 3102 | 42.040 | 9.260 | 61.670 | 1.00 | 24.41 |
| ATOM | 3856 | ND2 | ASN | 3102 | 41.630 | 8.236 | 63.645 | 1.00 | 26.00 |
| ATOM | 3857 | C | ASN | 3102 | 45.169 | 11.468 | 63.509 | 1.00 | 24.41 |
| ATOM | 3858 | O | ASN | 3102 | 44.554 | 12.525 | 63.530 | 1.00 | 24.40 |
| ATOM | 3859 | N | TYR | 3103 | 46.448 | 11.377 | 63.836 | 1.00 | 24.82 |
| ATOM | 3860 | CA | TYR | 3103 | 47.225 | 12.562 | 64.163 | 1.00 | 25.13 |
| ATOM | 3861 | CB | TYR | 3103 | 47.992 | 12.379 | 65.472 | 1.00 | 25.19 |
| ATOM | 3862 | CG | TYR | 3103 | 47.189 | 12.629 | 66.724 | 1.00 | 25.93 |
| ATOM | 3863 | CD1 | TYR | 3103 | 47.022 | 13.917 | 67.223 | 1.00 | 26.07 |
| ATOM | 3864 | CE1 | TYR | 3103 | 46.277 | 14.141 | 68.398 | 1.00 | 25.99 |
| ATOM | 3865 | CD2 | TYR | 3103 | 46.594 | 11.567 | 67.422 | 1.00 | 25.12 |
| ATOM | 3866 | CE2 | TYR | 3103 | 45.861 | 11.780 | 68.577 | 1.00 | 24.25 |
| ATOM | 3867 | CZ | TYR | 3103 | 45.703 | 13.062 | 69.065 | 1.00 | 25.13 |
| ATOM | 3868 | OH | TYR | 3103 | 44.980 | 13.263 | 70.225 | 1.00 | 25.77 |
| ATOM | 3869 | C | TYR | 3103 | 48.213 | 12.709 | 63.013 | 1.00 | 24.67 |
| ATOM | 3870 | O | TYR | 3103 | 48.493 | 11.744 | 62.312 | 1.00 | 25.44 |
| ATOM | 3871 | N | ASN | 3104 | 48.724 | 13.916 | 62.811 | 1.00 | 24.40 |
| ATOM | 3872 | CA | ASN | 3104 | 49.694 | 14.166 | 61.759 | 1.00 | 24.50 |
| ATOM | 3873 | CB | ASN | 3104 | 49.471 | 15.546 | 61.133 | 1.00 | 23.98 |
| ATOM | 3874 | CG | ASN | 3104 | 48.224 | 15.620 | 60.286 | 1.00 | 24.00 |
| ATOM | 3875 | OD1 | ASN | 3104 | 47.386 | 14.721 | 60.320 | 1.00 | 25.53 |
| ATOM | 3876 | ND2 | ASN | 3104 | 48.081 | 16.710 | 59.528 | 1.00 | 21.52 |
| ATOM | 3877 | C | ASN | 3104 | 51.076 | 14.163 | 62.372 | 1.00 | 25.15 |
| ATOM | 3878 | O | ASN | 3104 | 51.236 | 14.485 | 63.544 | 1.00 | 25.91 |
| ATOM | 3879 | N | THR | 3105 | 52.072 | 13.797 | 61.579 | 1.00 | 25.55 |
| ATOM | 3880 | CA | THR | 3105 | 53.459 | 13.830 | 62.027 | 1.00 | 26.28 |
| ATOM | 3881 | CB | THR | 3105 | 54.105 | 12.428 | 62.063 | 1.00 | 25.91 |
| ATOM | 3882 | OG1 | THR | 3105 | 53.921 | 11.791 | 60.794 | 1.00 | 25.63 |
| ATOM | 3883 | CG2 | THR | 3105 | 53.505 | 11.585 | 63.185 | 1.00 | 24.87 |
| ATOM | 3884 | C | THR | 3105 | 54.180 | 14.663 | 60.976 | 1.00 | 26.53 |
| ATOM | 3885 | O | THR | 3105 | 53.703 | 14.791 | 59.847 | 1.00 | 26.29 |
| ATOM | 3886 | N | TYR | 3106 | 55.315 | 15.236 | 61.347 | 1.00 | 26.47 |
| ATOM | 3887 | CA | TYR | 3106 | 56.091 | 16.043 | 60.413 | 1.00 | 27.45 |
| ATOM | 3888 | CB | TYR | 3106 | 55.866 | 17.527 | 60.695 | 1.00 | 26.05 |
| ATOM | 3889 | CG | TYR | 3106 | 54.435 | 17.933 | 60.441 | 1.00 | 24.62 |
| ATOM | 3890 | CD1 | TYR | 3106 | 54.032 | 18.390 | 59.187 | 1.00 | 24.91 |
| ATOM | 3891 | CE1 | TYR | 3106 | 52.699 | 18.715 | 58.930 | 1.00 | 24.06 |
| ATOM | 3892 | CD2 | TYR | 3106 | 53.468 | 17.812 | 61.439 | 1.00 | 24.62 |
| ATOM | 3893 | CE2 | TYR | 3106 | 52.131 | 18.135 | 61.193 | 1.00 | 23.99 |
| ATOM | 3894 | CZ | TYR | 3106 | 51.758 | 18.583 | 59.939 | 1.00 | 23.93 |
| ATOM | 3895 | OH | TYR | 3106 | 50.441 | 18.870 | 59.689 | 1.00 | 24.70 |
| ATOM | 3896 | C | TYR | 3106 | 57.547 | 15.651 | 60.553 | 1.00 | 28.19 |
| ATOM | 3897 | O | TYR | 3106 | 58.218 | 15.984 | 61.529 | 1.00 | 27.64 |
| ATOM | 3898 | N | ARG | 3107 | 58.018 | 14.918 | 59.555 | 1.00 | 29.69 |
| ATOM | 3899 | CA | ARG | 3107 | 59.379 | 14.403 | 59.547 | 1.00 | 30.82 |
| ATOM | 3900 | CB | ARG | 3107 | 59.334 | 12.951 | 59.069 | 1.00 | 30.17 |
| ATOM | 3901 | CG | ARG | 3107 | 60.441 | 12.032 | 59.559 | 1.00 | 31.02 |
| ATOM | 3902 | CD | ARG | 3107 | 60.213 | 10.629 | 58.995 | 1.00 | 30.69 |
| ATOM | 3903 | NE | ARG | 3107 | 59.902 | 10.724 | 57.567 | 1.00 | 32.60 |
| ATOM | 3904 | CZ | ARG | 3107 | 60.178 | 9.799 | 56.658 | 1.00 | 31.38 |
| ATOM | 3905 | NH1 | ARG | 3107 | 60.784 | 8.681 | 57.013 | 1.00 | 34.04 |
| ATOM | 3906 | NH2 | ARG | 3107 | 59.851 | 9.988 | 55.390 | 1.00 | 30.91 |
| ATOM | 3907 | C | ARG | 3107 | 60.290 | 15.238 | 58.654 | 1.00 | 30.92 |
| ATOM | 3908 | O | ARG | 3107 | 59.935 | 15.567 | 57.534 | 1.00 | 31.15 |
| ATOM | 3909 | N | SER | 3108 | 61.465 | 15.580 | 59.164 | 1.00 | 32.08 |
| ATOM | 3910 | CA | SER | 3108 | 62.434 | 16.375 | 58.411 | 1.00 | 33.43 |
| ATOM | 3911 | CB | SER | 3108 | 63.695 | 16.600 | 59.260 | 1.00 | 32.83 |
| ATOM | 3912 | OG | SER | 3108 | 64.718 | 17.281 | 58.545 | 1.00 | 31.25 |
| ATOM | 3913 | C | SER | 3108 | 62.825 | 15.665 | 57.119 | 1.00 | 34.57 |
| ATOM | 3914 | O | SER | 3108 | 63.228 | 14.505 | 57.154 | 1.00 | 34.51 |
| ATOM | 3915 | N | ARG | 3109 | 62.711 | 16.344 | 55.979 | 1.00 | 36.04 |
| ATOM | 3916 | CA | ARG | 3109 | 63.094 | 15.712 | 54.725 | 1.00 | 37.67 |
| ATOM | 3917 | CB | ARG | 3109 | 62.509 | 16.460 | 53.524 | 1.00 | 38.24 |
| ATOM | 3918 | CG | ARG | 3109 | 62.896 | 15.813 | 52.196 | 1.00 | 39.50 |
| ATOM | 3919 | CD | ARG | 3109 | 62.110 | 16.333 | 51.012 | 1.00 | 39.15 |
| ATOM | 3920 | NE | ARG | 3109 | 62.337 | 17.753 | 50.769 | 1.00 | 40.29 |
| ATOM | 3921 | CZ | ARG | 3109 | 62.119 | 18.349 | 49.600 | 1.00 | 41.48 |
| ATOM | 3922 | NH1 | ARG | 3109 | 61.672 | 17.637 | 48.571 | 1.00 | 42.35 |
| ATOM | 3923 | NH2 | ARG | 3109 | 62.339 | 19.653 | 49.460 | 1.00 | 41.75 |
| ATOM | 3924 | C | ARG | 3109 | 64.622 | 15.651 | 54.617 | 1.00 | 38.62 |
| ATOM | 3925 | O | ARG | 3109 | 65.178 | 14.827 | 53.890 | 1.00 | 38.19 |
| ATOM | 3926 | N | LYS | 3110 | 65.285 | 16.524 | 55.366 | 1.00 | 39.36 |
| ATOM | 3927 | CA | LYS | 3110 | 66.737 | 16.597 | 55.397 | 1.00 | 40.04 |
| ATOM | 3928 | CB | LYS | 3110 | 67.147 | 18.017 | 55.815 | 1.00 | 41.86 |
| ATOM | 3929 | CG | LYS | 3110 | 68.634 | 18.345 | 55.754 | 1.00 | 44.31 |
| ATOM | 3930 | CD | LYS | 3110 | 69.143 | 18.426 | 54.324 | 1.00 | 45.63 |
| ATOM | 3931 | CE | LYS | 3110 | 70.541 | 19.042 | 54.273 | 1.00 | 46.68 |
| ATOM | 3932 | NZ | LYS | 3110 | 71.015 | 19.245 | 52.856 | 1.00 | 47.70 |
| ATOM | 3933 | C | LYS | 3110 | 67.266 | 15.561 | 56.394 | 1.00 | 39.65 |
| ATOM | 3934 | O | LYS | 3110 | 68.118 | 14.738 | 56.050 | 1.00 | 40.14 |
| ATOM | 3935 | N | TYR | 3111 | 66.757 | 15.606 | 57.626 | 1.00 | 38.50 |
| ATOM | 3936 | CA | TYR | 3111 | 67.157 | 14.666 | 58.680 | 1.00 | 37.63 |
| ATOM | 3937 | CB | TYR | 3111 | 67.426 | 15.430 | 59.971 | 1.00 | 36.83 |
| ATOM | 3938 | CG | TYR | 3111 | 68.276 | 16.652 | 59.732 | 1.00 | 36.21 |
| ATOM | 3939 | CD1 | TYR | 3111 | 69.568 | 16.532 | 59.219 | 1.00 | 36.18 |
| ATOM | 3940 | CE1 | TYR | 3111 | 70.328 | 17.661 | 58.906 | 1.00 | 36.05 |
| ATOM | 3941 | CD2 | TYR | 3111 | 67.763 | 17.935 | 59.938 | 1.00 | 35.57 |
| ATOM | 3942 | CE2 | TYR | 3111 | 68.506 | 19.066 | 59.632 | 1.00 | 36.03 |
| ATOM | 3943 | CZ | TYR | 3111 | 69.789 | 18.922 | 59.111 | 1.00 | 36.25 |
| ATOM | 3944 | OH | TYR | 3111 | 70.519 | 20.034 | 58.770 | 1.00 | 35.86 |
| ATOM | 3945 | C | TYR | 3111 | 65.997 | 13.688 | 58.848 | 1.00 | 37.77 |
| ATOM | 3946 | O | TYR | 3111 | 65.262 | 13.712 | 59.841 | 1.00 | 37.66 |
| ATOM | 3947 | N | THR | 3112 | 65.870 | 12.816 | 57.852 | 1.00 | 37.24 |
| ATOM | 3948 | CA | THR | 3112 | 64.793 | 11.843 | 57.738 | 1.00 | 36.60 |
| ATOM | 3949 | CB | THR | 3112 | 65.061 | 10.850 | 56.619 | 1.00 | 35.66 |
| ATOM | 3950 | OG1 | THR | 3112 | 65.949 | 9.840 | 57.098 | 1.00 | 35.28 |
| ATOM | 3951 | CG2 | THR | 3112 | 65.654 | 11.553 | 55.416 | 1.00 | 34.43 |
| ATOM | 3952 | C | THR | 3112 | 64.337 | 11.014 | 58.919 | 1.00 | 37.07 |
| ATOM | 3953 | O | THR | 3112 | 63.324 | 10.327 | 58.814 | 1.00 | 37.86 |
| ATOM | 3954 | N | SER | 3113 | 65.037 | 11.068 | 60.039 | 1.00 | 36.47 |
| ATOM | 3955 | CA | SER | 3113 | 64.629 | 10.262 | 61.177 | 1.00 | 35.58 |
| ATOM | 3956 | CB | SER | 3113 | 65.819 | 9.396 | 61.585 | 1.00 | 35.86 |
| ATOM | 3957 | OG | SER | 3113 | 65.426 | 8.317 | 62.393 | 1.00 | 35.93 |
| ATOM | 3958 | C | SER | 3113 | 64.168 | 11.128 | 62.349 | 1.00 | 35.14 |
| ATOM | 3959 | O | SER | 3113 | 63.840 | 10.622 | 63.426 | 1.00 | 34.97 |
| ATOM | 3960 | N | TRP | 3114 | 64.135 | 12.438 | 62.131 | 1.00 | 34.37 |
| ATOM | 3961 | CA | TRP | 3114 | 63.746 | 13.366 | 63.182 | 1.00 | 34.06 |
| ATOM | 3962 | CB | TRP | 3114 | 64.836 | 14.428 | 63.324 | 1.00 | 33.76 |
| ATOM | 3963 | CG | TRP | 3114 | 66.189 | 13.837 | 63.661 | 1.00 | 34.72 |
| ATOM | 3964 | CD2 | TRP | 3114 | 67.456 | 14.511 | 63.655 | 1.00 | 34.84 |
| ATOM | 3965 | CE2 | TRP | 3114 | 68.433 | 13.571 | 64.064 | 1.00 | 35.18 |
| ATOM | 3966 | CE3 | TRP | 3114 | 67.860 | 15.814 | 63.347 | 1.00 | 34.57 |
| ATOM | 3967 | CD1 | TRP | 3114 | 66.448 | 12.553 | 64.061 | 1.00 | 35.01 |
| ATOM | 3968 | NE1 | TRP | 3114 | 67.790 | 12.388 | 64.305 | 1.00 | 34.91 |
| ATOM | 3969 | CZ2 | TRP | 3114 | 69.781 | 13.893 | 64.170 | 1.00 | 35.23 |
| ATOM | 3970 | CZ3 | TRP | 3114 | 69.200 | 16.136 | 63.454 | 1.00 | 35.17 |
| ATOM | 3971 | CH2 | TRP | 3114 | 70.146 | 15.178 | 63.862 | 1.00 | 35.91 |
| ATOM | 3972 | C | TRP | 3114 | 62.387 | 14.023 | 62.940 | 1.00 | 33.40 |
| ATOM | 3973 | O | TRP | 3114 | 62.073 | 14.425 | 61.814 | 1.00 | 33.19 |
| ATOM | 3974 | N | TYR | 3115 | 61.582 | 14.130 | 63.998 | 1.00 | 32.09 |
| ATOM | 3975 | CA | TYR | 3115 | 60.257 | 14.729 | 63.878 | 1.00 | 30.70 |
| ATOM | 3976 | CB | TYR | 3115 | 59.170 | 13.825 | 64.473 | 1.00 | 28.03 |
| ATOM | 3977 | CG | TYR | 3115 | 58.978 | 12.485 | 63.818 | 1.00 | 26.86 |
| ATOM | 3978 | CD1 | TYR | 3115 | 59.762 | 11.392 | 64.183 | 1.00 | 26.65 |
| ATOM | 3979 | CE1 | TYR | 3115 | 59.578 | 10.132 | 63.596 | 1.00 | 26.07 |
| ATOM | 3980 | CD2 | TYR | 3115 | 57.995 | 12.297 | 62.838 | 1.00 | 26.89 |
| ATOM | 3981 | CE2 | TYR | 3115 | 57.800 | 11.047 | 62.238 | 1.00 | 26.32 |
| ATOM | 3982 | CZ | TYR | 3115 | 58.600 | 9.970 | 62.628 | 1.00 | 26.19 |
| ATOM | 3983 | OH | TYR | 3115 | 58.432 | 8.735 | 62.054 | 1.00 | 26.22 |
| ATOM | 3984 | C | TYR | 3115 | 60.142 | 16.062 | 64.578 | 1.00 | 30.58 |
| ATOM | 3985 | O | TYR | 3115 | 60.876 | 16.346 | 65.530 | 1.00 | 30.75 |
| ATOM | 3986 | N | VAL | 3116 | 59.209 | 16.882 | 64.099 | 1.00 | 30.13 |
| ATOM | 3987 | CA | VAL | 3116 | 58.930 | 18.157 | 64.741 | 1.00 | 29.55 |
| ATOM | 3988 | CB | VAL | 3116 | 57.985 | 18.992 | 63.894 | 1.00 | 28.14 |
| ATOM | 3989 | CG1 | VAL | 3116 | 57.550 | 20.227 | 64.657 | 1.00 | 27.32 |
| ATOM | 3990 | CG2 | VAL | 3116 | 58.664 | 19.356 | 62.607 | 1.00 | 27.08 |
| ATOM | 3991 | C | VAL | 3116 | 58.225 | 17.711 | 66.029 | 1.00 | 30.22 |
| ATOM | 3992 | O | VAL | 3116 | 57.377 | 16.821 | 65.995 | 1.00 | 30.55 |
| ATOM | 3993 | N | ALA | 3117 | 58.572 | 18.308 | 67.161 | 1.00 | 30.84 |
| ATOM | 3994 | CA | ALA | 3117 | 57.970 | 17.889 | 68.413 | 1.00 | 31.30 |
| ATOM | 3995 | CB | ALA | 3117 | 58.678 | 16.645 | 68.896 | 1.00 | 30.07 |
| ATOM | 3996 | C | ALA | 3117 | 57.993 | 18.942 | 69.508 | 1.00 | 32.31 |
| ATOM | 3997 | O | ALA | 3117 | 58.860 | 19.810 | 69.517 | 1.00 | 32.57 |
| ATOM | 3998 | N | LEU | 3118 | 57.036 | 18.849 | 70.431 | 1.00 | 33.48 |
| ATOM | 3999 | CA | LEU | 3118 | 56.949 | 19.761 | 71.563 | 1.00 | 34.88 |
| ATOM | 4000 | CB | LEU | 3118 | 55.666 | 20.592 | 71.506 | 1.00 | 34.02 |
| ATOM | 4001 | CG | LEU | 3118 | 55.466 | 21.592 | 70.356 | 1.00 | 33.90 |
| ATOM | 4002 | CD1 | LEU | 3118 | 54.215 | 22.414 | 70.634 | 1.00 | 33.16 |
| ATOM | 4003 | CD2 | LEU | 3118 | 56.670 | 22.518 | 70.225 | 1.00 | 32.79 |
| ATOM | 4004 | C | LEU | 3118 | 56.953 | 18.943 | 72.846 | 1.00 | 36.89 |
| ATOM | 4005 | O | LEU | 3118 | 56.347 | 17.879 | 72.897 | 1.00 | 37.67 |
| ATOM | 4006 | N | LYS | 3119 | 57.650 | 19.431 | 73.871 | 1.00 | 38.85 |
| ATOM | 4007 | CA | LYS | 3119 | 57.707 | 18.747 | 75.158 | 1.00 | 40.71 |
| ATOM | 4008 | CB | LYS | 3119 | 58.970 | 19.149 | 75.934 | 1.00 | 42.18 |
| ATOM | 4009 | CG | LYS | 3119 | 60.263 | 18.648 | 75.320 | 1.00 | 44.83 |
| ATOM | 4010 | CD | LYS | 3119 | 61.406 | 18.546 | 76.334 | 1.00 | 46.30 |
| ATOM | 4011 | CE | LYS | 3119 | 61.898 | 19.910 | 76.790 | 1.00 | 48.37 |
| ATOM | 4012 | NZ | LYS | 3119 | 63.178 | 19.836 | 77.585 | 1.00 | 48.92 |
| ATOM | 4013 | C | LYS | 3119 | 56.476 | 19.141 | 75.964 | 1.00 | 41.75 |
| ATOM | 4014 | O | LYS | 3119 | 55.757 | 20.062 | 75.590 | 1.00 | 41.29 |
| ATOM | 4015 | N | ARG | 3120 | 56.237 | 18.443 | 77.069 | 1.00 | 43.43 |
| ATOM | 4016 | CA | ARG | 3120 | 55.097 | 18.737 | 77.939 | 1.00 | 45.49 |
| ATOM | 4017 | CB | ARG | 3120 | 55.022 | 17.720 | 79.084 | 1.00 | 47.02 |
| ATOM | 4018 | CG | ARG | 3120 | 54.725 | 16.288 | 78.680 | 1.00 | 50.54 |
| ATOM | 4019 | CD | ARG | 3120 | 54.856 | 15.353 | 79.892 | 1.00 | 53.97 |
| ATOM | 4020 | NE | ARG | 3120 | 54.382 | 13.990 | 79.629 | 1.00 | 56.91 |
| ATOM | 4021 | CZ | ARG | 3120 | 54.543 | 12.964 | 80.467 | 1.00 | 58.18 |
| ATOM | 4022 | NH1 | ARG | 3120 | 55.175 | 13.145 | 81.628 | 1.00 | 57.86 |
| ATOM | 4023 | NH2 | ARG | 3120 | 54.068 | 11.757 | 80.145 | 1.00 | 58.75 |
| ATOM | 4024 | C | ARG | 3120 | 55.213 | 20.139 | 78.550 | 1.00 | 45.57 |
| ATOM | 4025 | O | ARG | 3120 | 54.240 | 20.665 | 79.099 | 1.00 | 45.78 |
| ATOM | 4026 | N | THR | 3121 | 56.402 | 20.738 | 78.463 | 1.00 | 45.18 |
| ATOM | 4027 | CA | THR | 3121 | 56.628 | 22.067 | 79.027 | 1.00 | 43.90 |
| ATOM | 4028 | CB | THR | 3121 | 58.092 | 22.278 | 79.410 | 1.00 | 43.45 |
| ATOM | 4029 | OG1 | THR | 3121 | 58.898 | 22.262 | 78.228 | 1.00 | 43.56 |
| ATOM | 4030 | CG2 | THR | 3121 | 58.560 | 21.190 | 80.342 | 1.00 | 43.10 |
| ATOM | 4031 | C | THR | 3121 | 56.257 | 23.172 | 78.061 | 1.00 | 43.77 |
| ATOM | 4032 | O | THR | 3121 | 56.225 | 24.335 | 78.429 | 1.00 | 43.80 |
| ATOM | 4033 | N | GLY | 3122 | 55.975 | 22.811 | 76.819 | 1.00 | 44.12 |
| ATOM | 4034 | CA | GLY | 3122 | 55.633 | 23.820 | 75.837 | 1.00 | 43.82 |
| ATOM | 4035 | C | GLY | 3122 | 56.854 | 24.228 | 75.036 | 1.00 | 43.46 |
| ATOM | 4036 | O | GLY | 3122 | 56.759 | 25.036 | 74.119 | 1.00 | 43.56 |
| ATOM | 4037 | N | GLN | 3123 | 58.006 | 23.683 | 75.399 | 1.00 | 43.18 |
| ATOM | 4038 | CA | GLN | 3123 | 59.242 | 23.965 | 74.687 | 1.00 | 43.56 |
| ATOM | 4039 | CB | GLN | 3123 | 60.438 | 23.870 | 75.630 | 1.00 | 43.92 |
| ATOM | 4040 | CG | GLN | 3123 | 60.469 | 24.946 | 76.669 | 1.00 | 44.98 |
| ATOM | 4041 | CD | GLN | 3123 | 60.512 | 26.322 | 76.045 | 1.00 | 46.14 |
| ATOM | 4042 | OE1 | GLN | 3123 | 61.441 | 26.653 | 75.303 | 1.00 | 46.76 |
| ATOM | 4043 | NE2 | GLN | 3123 | 59.505 | 27.135 | 76.336 | 1.00 | 46.90 |
| ATOM | 4044 | C | GLN | 3123 | 59.372 | 22.907 | 73.606 | 1.00 | 43.11 |
| ATOM | 4045 | O | GLN | 3123 | 58.953 | 21.770 | 73.802 | 1.00 | 43.38 |
| ATOM | 4046 | N | TYR | 3124 | 59.949 | 23.267 | 72.468 | 1.00 | 42.36 |
| ATOM | 4047 | CA | TYR | 3124 | 60.102 | 22.298 | 71.399 | 1.00 | 42.01 |
| ATOM | 4048 | CB | TYR | 3124 | 60.657 | 22.981 | 70.142 | 1.00 | 41.81 |
| ATOM | 4049 | CG | TYR | 3124 | 62.136 | 23.312 | 70.187 | 1.00 | 41.67 |
| ATOM | 4050 | CD1 | TYR | 3124 | 63.097 | 22.313 | 70.021 | 1.00 | 41.76 |
| ATOM | 4051 | CE1 | TYR | 3124 | 64.456 | 22.606 | 70.054 | 1.00 | 42.16 |
| ATOM | 4052 | CD2 | TYR | 3124 | 62.574 | 24.623 | 70.390 | 1.00 | 41.60 |
| ATOM | 4053 | CE2 | TYR | 3124 | 63.932 | 24.931 | 70.427 | 1.00 | 41.64 |
| ATOM | 4054 | CZ | TYR | 3124 | 64.870 | 23.917 | 70.258 | 1.00 | 42.45 |
| ATOM | 4055 | OH | TYR | 3124 | 66.221 | 24.203 | 70.295 | 1.00 | 42.72 |
| ATOM | 4056 | C | TYR | 3124 | 61.043 | 21.205 | 71.876 | 1.00 | 41.62 |
| ATOM | 4057 | O | TYR | 3124 | 61.831 | 21.424 | 72.788 | 1.00 | 42.04 |
| ATOM | 4058 | N | LYS | 3125 | 60.953 | 20.031 | 71.266 | 1.00 | 41.16 |
| ATOM | 4059 | CA | LYS | 3125 | 61.810 | 18.914 | 71.631 | 1.00 | 40.81 |
| ATOM | 4060 | CB | LYS | 3125 | 60.956 | 17.674 | 71.900 | 1.00 | 39.63 |
| ATOM | 4061 | CG | LYS | 3125 | 61.740 | 16.428 | 72.229 | 1.00 | 38.43 |
| ATOM | 4062 | CD | LYS | 3125 | 60.819 | 15.299 | 72.630 | 1.00 | 37.98 |
| ATOM | 4063 | CE | LYS | 3125 | 61.606 | 14.045 | 72.984 | 1.00 | 38.56 |
| ATOM | 4064 | NZ | LYS | 3125 | 60.771 | 12.994 | 73.632 | 1.00 | 36.78 |
| ATOM | 4065 | C | LYS | 3125 | 62.793 | 18.646 | 70.496 | 1.00 | 41.13 |
| ATOM | 4066 | O | LYS | 3125 | 62.401 | 18.593 | 69.334 | 1.00 | 41.25 |
| ATOM | 4067 | N | LEU | 3126 | 64.070 | 18.500 | 70.833 | 1.00 | 41.40 |
| ATOM | 4068 | CA | LEU | 3126 | 65.098 | 18.233 | 69.838 | 1.00 | 41.51 |
| ATOM | 4069 | CB | LEU | 3126 | 66.416 | 17.905 | 70.533 | 1.00 | 41.76 |
| ATOM | 4070 | CG | LEU | 3126 | 67.049 | 19.067 | 71.296 | 1.00 | 42.64 |
| ATOM | 4071 | CD1 | LEU | 3126 | 68.235 | 18.565 | 72.099 | 1.00 | 42.24 |
| ATOM | 4072 | CD2 | LEU | 3126 | 67.473 | 20.147 | 70.313 | 1.00 | 42.13 |
| ATOM | 4073 | C | LEU | 3126 | 64.709 | 17.076 | 68.924 | 1.00 | 41.69 |
| ATOM | 4074 | O | LEU | 3126 | 64.354 | 15.996 | 69.397 | 1.00 | 42.10 |
| ATOM | 4075 | N | GLY | 3127 | 64.781 | 17.301 | 67.616 | 1.00 | 41.22 |
| ATOM | 4076 | CA | GLY | 3127 | 64.439 | 16.253 | 66.679 | 1.00 | 41.14 |
| ATOM | 4077 | C | GLY | 3127 | 65.301 | 15.028 | 66.899 | 1.00 | 41.67 |
| ATOM | 4078 | O | GLY | 3127 | 64.864 | 13.895 | 66.686 | 1.00 | 41.09 |
| ATOM | 4079 | N | SER | 3128 | 66.535 | 15.259 | 67.336 | 1.00 | 42.01 |
| ATOM | 4080 | CA | SER | 3128 | 67.473 | 14.172 | 67.578 | 1.00 | 42.37 |
| ATOM | 4081 | CB | SER | 3128 | 68.845 | 14.746 | 67.930 | 1.00 | 42.23 |
| ATOM | 4082 | OG | SER | 3128 | 68.801 | 15.447 | 69.160 | 1.00 | 41.94 |
| ATOM | 4083 | C | SER | 3128 | 66.993 | 13.267 | 68.709 | 1.00 | 42.59 |
| ATOM | 4084 | O | SER | 3128 | 67.455 | 12.140 | 68.851 | 1.00 | 41.75 |
| ATOM | 4085 | N | LYS | 3129 | 66.056 | 13.769 | 69.504 | 1.00 | 43.34 |
| ATOM | 4086 | CA | LYS | 3129 | 65.532 | 13.022 | 70.642 | 1.00 | 44.33 |
| ATOM | 4087 | CB | LYS | 3129 | 65.479 | 13.952 | 71.876 | 1.00 | 45.33 |
| ATOM | 4088 | CG | LYS | 3129 | 65.888 | 13.310 | 73.208 | 1.00 | 48.32 |
| ATOM | 4089 | CD | LYS | 3129 | 64.848 | 13.517 | 74.349 | 1.00 | 50.22 |
| ATOM | 4090 | CE | LYS | 3129 | 64.795 | 14.969 | 74.876 | 1.00 | 51.56 |
| ATOM | 4091 | NZ | LYS | 3129 | 63.774 | 15.190 | 75.960 | 1.00 | 51.30 |
| ATOM | 4092 | C | LYS | 3129 | 64.135 | 12.458 | 70.359 | 1.00 | 43.96 |
| ATOM | 4093 | O | LYS | 3129 | 63.532 | 11.830 | 71.231 | 1.00 | 44.17 |
| ATOM | 4094 | N | THR | 3130 | 63.622 | 12.676 | 69.149 | 1.00 | 43.04 |
| ATOM | 4095 | CA | THR | 3130 | 62.284 | 12.201 | 68.803 | 1.00 | 42.15 |
| ATOM | 4096 | CB | THR | 3130 | 61.622 | 13.122 | 67.759 | 1.00 | 41.22 |
| ATOM | 4097 | OG1 | THR | 3130 | 62.384 | 13.093 | 66.548 | 1.00 | 40.88 |
| ATOM | 4098 | CG2 | THR | 3130 | 61.549 | 14.551 | 68.275 | 1.00 | 40.27 |
| ATOM | 4099 | C | THR | 3130 | 62.282 | 10.771 | 68.275 | 1.00 | 42.16 |
| ATOM | 4100 | O | THR | 3130 | 63.329 | 10.210 | 67.959 | 1.00 | 42.40 |
| ATOM | 4101 | N | GLY | 3131 | 61.095 | 10.184 | 68.193 | 1.00 | 41.80 |
| ATOM | 4102 | CA | GLY | 3131 | 60.963 | 8.827 | 67.701 | 1.00 | 41.29 |
| ATOM | 4103 | C | GLY | 3131 | 59.516 | 8.552 | 67.345 | 1.00 | 41.14 |
| ATOM | 4104 | O | GLY | 3131 | 58.632 | 9.306 | 67.757 | 1.00 | 40.85 |
| ATOM | 4105 | N | PRO | 3132 | 59.235 | 7.484 | 66.581 | 1.00 | 41.29 |
| ATOM | 4106 | CD | PRO | 3132 | 60.210 | 6.577 | 65.957 | 1.00 | 41.74 |
| ATOM | 4107 | CA | PRO | 3132 | 57.873 | 7.126 | 66.175 | 1.00 | 41.21 |
| ATOM | 4108 | CB | PRO | 3132 | 58.088 | 5.937 | 65.233 | 1.00 | 41.09 |
| ATOM | 4109 | CG | PRO | 3132 | 59.367 | 5.354 | 65.693 | 1.00 | 41.75 |
| ATOM | 4110 | C | PRO | 3132 | 56.884 | 6.829 | 67.302 | 1.00 | 41.23 |
| ATOM | 4111 | O | PRO | 3132 | 55.699 | 7.157 | 67.192 | 1.00 | 41.41 |
| ATOM | 4112 | N | GLY | 3133 | 57.361 | 6.227 | 68.386 | 1.00 | 40.72 |
| ATOM | 4113 | CA | GLY | 3133 | 56.469 | 5.917 | 69.488 | 1.00 | 40.27 |
| ATOM | 4114 | C | GLY | 3133 | 56.308 | 7.018 | 70.521 | 1.00 | 39.61 |
| ATOM | 4115 | O | GLY | 3133 | 56.019 | 6.734 | 71.684 | 1.00 | 40.41 |
| ATOM | 4116 | N | GLN | 3134 | 56.476 | 8.271 | 70.113 | 1.00 | 38.20 |
| ATOM | 4117 | CA | GLN | 3134 | 56.357 | 9.374 | 71.053 | 1.00 | 37.29 |
| ATOM | 4118 | CB | GLN | 3134 | 57.595 | 10.259 | 70.977 | 1.00 | 36.56 |
| ATOM | 4119 | CG | GLN | 3134 | 58.879 | 9.556 | 71.346 | 1.00 | 35.59 |
| ATOM | 4120 | CD | GLN | 3134 | 60.042 | 10.513 | 71.353 | 1.00 | 36.21 |
| ATOM | 4121 | OE1 | GLN | 3134 | 61.191 | 10.117 | 71.528 | 1.00 | 36.13 |
| ATOM | 4122 | NE2 | GLN | 3134 | 59.749 | 11.790 | 71.161 | 1.00 | 35.89 |
| ATOM | 4123 | C | GLN | 3134 | 55.117 | 10.227 | 70.849 | 1.00 | 37.01 |
| ATOM | 4124 | O | GLN | 3134 | 54.692 | 10.480 | 69.733 | 1.00 | 37.74 |
| ATOM | 4125 | N | LYS | 3135 | 54.551 | 10.674 | 71.956 | 1.00 | 36.45 |
| ATOM | 4126 | CA | LYS | 3135 | 53.363 | 11.507 | 71.969 | 1.00 | 35.39 |
| ATOM | 4127 | CB | LYS | 3135 | 52.826 | 11.513 | 73.396 | 1.00 | 35.38 |
| ATOM | 4128 | CG | LYS | 3135 | 51.563 | 12.276 | 73.664 | 1.00 | 35.73 |
| ATOM | 4129 | CD | LYS | 3135 | 51.176 | 11.987 | 75.102 | 1.00 | 36.00 |
| ATOM | 4130 | CE | LYS | 3135 | 49.882 | 12.643 | 75.510 | 1.00 | 37.40 |
| ATOM | 4131 | NZ | LYS | 3135 | 49.652 | 12.408 | 76.968 | 1.00 | 38.21 |
| ATOM | 4132 | C | LYS | 3135 | 53.701 | 12.925 | 71.520 | 1.00 | 35.02 |
| ATOM | 4133 | O | LYS | 3135 | 52.839 | 13.650 | 71.028 | 1.00 | 35.67 |
| ATOM | 4134 | N | ALA | 3136 | 54.967 | 13.302 | 71.683 | 1.00 | 33.63 |
| ATOM | 4135 | CA | ALA | 3136 | 55.451 | 14.628 | 71.332 | 1.00 | 32.46 |
| ATOM | 4136 | CB | ALA | 3136 | 56.836 | 14.820 | 71.909 | 1.00 | 32.39 |
| ATOM | 4137 | C | ALA | 3136 | 55.468 | 14.961 | 69.839 | 1.00 | 32.06 |
| ATOM | 4138 | O | ALA | 3136 | 55.460 | 16.139 | 69.460 | 1.00 | 32.00 |
| ATOM | 4139 | N | ILE | 3137 | 55.486 | 13.942 | 68.986 | 1.00 | 30.71 |
| ATOM | 4140 | CA | ILE | 3137 | 55.522 | 14.197 | 67.549 | 1.00 | 29.93 |
| ATOM | 4141 | CB | ILE | 3137 | 56.356 | 13.132 | 66.809 | 1.00 | 28.69 |
| ATOM | 4142 | CG2 | ILE | 3137 | 57.734 | 12.991 | 67.469 | 1.00 | 26.72 |
| ATOM | 4143 | CG1 | ILE | 3137 | 55.587 | 11.813 | 66.775 | 1.00 | 27.75 |
| ATOM | 4144 | CD1 | ILE | 3137 | 56.247 | 10.726 | 65.958 | 1.00 | 26.34 |
| ATOM | 4145 | C | ILE | 3137 | 54.149 | 14.260 | 66.880 | 1.00 | 30.23 |
| ATOM | 4146 | O | ILE | 3137 | 54.053 | 14.573 | 65.695 | 1.00 | 30.59 |
| ATOM | 4147 | N | LEU | 3138 | 53.095 | 13.982 | 67.641 | 1.00 | 29.89 |
| ATOM | 4148 | CA | LEU | 3138 | 51.742 | 13.971 | 67.099 | 1.00 | 29.30 |
| ATOM | 4149 | CB | LEU | 3138 | 50.913 | 12.926 | 67.835 | 1.00 | 28.78 |
| ATOM | 4150 | CG | LEU | 3138 | 51.579 | 11.552 | 67.799 | 1.00 | 28.96 |
| ATOM | 4151 | CD1 | LEU | 3138 | 50.814 | 10.552 | 68.659 | 1.00 | 28.80 |
| ATOM | 4152 | CD2 | LEU | 3138 | 51.638 | 11.093 | 66.343 | 1.00 | 30.05 |
| ATOM | 4153 | C | LEU | 3138 | 51.014 | 15.308 | 67.146 | 1.00 | 29.53 |
| ATOM | 4154 | O | LEU | 3138 | 50.895 | 15.931 | 68.204 | 1.00 | 29.55 |
| ATOM | 4155 | N | PHE | 3139 | 50.506 | 15.746 | 65.998 | 1.00 | 28.97 |
| ATOM | 4156 | CA | PHE | 3139 | 49.777 | 17.006 | 65.948 | 1.00 | 28.49 |
| ATOM | 4157 | CB | PHE | 3139 | 50.557 | 18.075 | 65.188 | 1.00 | 27.65 |
| ATOM | 4158 | CG | PHE | 3139 | 51.850 | 18.436 | 65.822 | 1.00 | 25.80 |
| ATOM | 4159 | CD1 | PHE | 3139 | 52.962 | 17.632 | 65.654 | 1.00 | 25.85 |
| ATOM | 4160 | CD2 | PHE | 3139 | 51.949 | 19.567 | 66.615 | 1.00 | 26.05 |
| ATOM | 4161 | CE1 | PHE | 3139 | 54.152 | 17.948 | 66.265 | 1.00 | 26.07 |
| ATOM | 4162 | CE2 | PHE | 3139 | 53.136 | 19.892 | 67.229 | 1.00 | 25.33 |
| ATOM | 4163 | CZ | PHE | 3139 | 54.239 | 19.083 | 67.056 | 1.00 | 26.10 |
| ATOM | 4164 | C | PHE | 3139 | 48.428 | 16.864 | 65.301 | 1.00 | 29.32 |
| ATOM | 4165 | O | PHE | 3139 | 48.255 | 16.120 | 64.342 | 1.00 | 29.93 |
| ATOM | 4166 | N | LEU | 3140 | 47.473 | 17.610 | 65.826 | 1.00 | 29.75 |
| ATOM | 4167 | CA | LEU | 3140 | 46.125 | 17.583 | 65.310 | 1.00 | 30.88 |
| ATOM | 4168 | CB | LEU | 3140 | 45.146 | 17.408 | 66.473 | 1.00 | 30.22 |
| ATOM | 4169 | CG | LEU | 3140 | 43.700 | 17.032 | 66.153 | 1.00 | 29.88 |
| ATOM | 4170 | CD1 | LEU | 3140 | 43.695 | 15.831 | 65.243 | 1.00 | 29.65 |
| ATOM | 4171 | CD2 | LEU | 3140 | 42.941 | 16.735 | 67.434 | 1.00 | 29.30 |
| ATOM | 4172 | C | LEU | 3140 | 45.900 | 18.910 | 64.608 | 1.00 | 32.12 |
| ATOM | 4173 | O | LEU | 3140 | 45.879 | 19.953 | 65.250 | 1.00 | 32.46 |
| ATOM | 4174 | N | PRO | 3141 | 45.753 | 18.891 | 63.277 | 1.00 | 32.93 |
| ATOM | 4175 | CD | PRO | 3141 | 45.750 | 17.734 | 62.366 | 1.00 | 33.53 |
| ATOM | 4176 | CA | PRO | 3141 | 45.535 | 20.134 | 62.542 | 1.00 | 34.24 |
| ATOM | 4177 | CB | PRO | 3141 | 45.755 | 19.710 | 61.095 | 1.00 | 33.67 |
| ATOM | 4178 | CG | PRO | 3141 | 45.177 | 18.324 | 61.093 | 1.00 | 33.64 |
| ATOM | 4179 | C | PRO | 3141 | 44.135 | 20.682 | 62.791 | 1.00 | 35.81 |
| ATOM | 4180 | O | PRO | 3141 | 43.158 | 19.944 | 62.758 | 1.00 | 35.58 |
| ATOM | 4181 | N | MET | 3142 | 44.046 | 21.978 | 63.056 | 1.00 | 37.82 |
| ATOM | 4182 | CA | MET | 3142 | 42.762 | 22.625 | 63.296 | 1.00 | 40.51 |
| ATOM | 4183 | CB | MET | 3142 | 42.628 | 23.014 | 64.757 | 1.00 | 39.85 |
| ATOM | 4184 | CG | MET | 3142 | 42.870 | 21.894 | 65.702 | 1.00 | 41.10 |
| ATOM | 4185 | SD | MET | 3142 | 42.525 | 22.404 | 67.380 | 1.00 | 42.03 |
| ATOM | 4186 | CE | MET | 3142 | 40.829 | 22.046 | 67.466 | 1.00 | 42.50 |
| ATOM | 4187 | C | MET | 3142 | 42.698 | 23.887 | 62.464 | 1.00 | 42.36 |
| ATOM | 4188 | O | MET | 3142 | 43.715 | 24.544 | 62.260 | 1.00 | 42.88 |
| ATOM | 4189 | N | SER | 3143 | 41.515 | 24.240 | 61.985 | 1.00 | 44.85 |
| ATOM | 4190 | CA | SER | 3143 | 41.394 | 25.452 | 61.194 | 1.00 | 47.93 |
| ATOM | 4191 | CB | SER | 3143 | 39.985 | 25.599 | 60.633 | 1.00 | 48.53 |
| ATOM | 4192 | OG | SER | 3143 | 39.051 | 25.849 | 61.670 | 1.00 | 50.73 |
| ATOM | 4193 | C | SER | 3143 | 41.715 | 26.632 | 62.101 | 1.00 | 49.87 |
| ATOM | 4194 | O | SER | 3143 | 41.570 | 26.548 | 63.328 | 1.00 | 49.27 |
| ATOM | 4195 | N | ALA | 3144 | 42.179 | 27.720 | 61.496 | 1.00 | 52.56 |
| ATOM | 4196 | CA | ALA | 3144 | 42.517 | 28.920 | 62.248 | 1.00 | 55.28 |
| ATOM | 4197 | CB | ALA | 3144 | 43.839 | 29.490 | 61.769 | 1.00 | 54.86 |
| ATOM | 4198 | C | ALA | 3144 | 41.390 | 29.896 | 61.987 | 1.00 | 57.64 |
| ATOM | 4199 | O | ALA | 3144 | 41.372 | 30.592 | 60.976 | 1.00 | 58.54 |
| ATOM | 4200 | N | LYS | 3145 | 40.404 | 29.880 | 62.869 | 1.00 | 60.39 |
| ATOM | 4201 | CA | LYS | 3145 | 39.257 | 30.751 | 62.729 | 1.00 | 62.90 |
| ATOM | 4202 | CB | LYS | 3145 | 38.129 | 30.018 | 61.977 | 1.00 | 64.47 |
| ATOM | 4203 | CG | LYS | 3145 | 38.583 | 29.441 | 60.617 | 1.00 | 66.36 |
| ATOM | 4204 | CD | LYS | 3145 | 37.443 | 28.879 | 59.756 | 1.00 | 67.43 |
| ATOM | 4205 | CE | LYS | 3145 | 37.979 | 28.357 | 58.411 | 1.00 | 67.94 |
| ATOM | 4206 | NZ | LYS | 3145 | 36.907 | 27.887 | 57.477 | 1.00 | 68.21 |
| ATOM | 4207 | C | LYS | 3145 | 38.854 | 31.126 | 64.144 | 1.00 | 63.85 |
| ATOM | 4208 | O | LYS | 3145 | 38.323 | 30.297 | 64.895 | 1.00 | 63.55 |
| ATOM | 4209 | N | ALA | 3146 | 39.153 | 32.379 | 64.487 | 1.00 | 64.80 |
| ATOM | 4210 | CA | ALA | 3146 | 38.897 | 32.983 | 65.796 | 1.00 | 66.21 |
| ATOM | 4211 | CB | ALA | 3146 | 38.147 | 32.019 | 66.736 | 1.00 | 66.07 |
| ATOM | 4212 | C | ALA | 3146 | 40.254 | 33.341 | 66.397 | 1.00 | 66.79 |
| ATOM | 4213 | O | ALA | 3146 | 41.256 | 33.074 | 65.694 | 1.00 | 67.66 |
| ATOM | 4214 | CB | MSE | 2149 | 27.593 | 19.576 | −21.743 | 1.00 | 75.07 |
| ATOM | 4215 | CG | MSE | 2149 | 26.822 | 20.830 | −21.312 | 1.00 | 78.40 |
| ATOM | 4216 | SE | MSE | 2149 | 26.886 | 22.246 | −22.467 | 1.00 | 83.46 |
| ATOM | 4217 | CE | MSE | 2149 | 25.367 | 21.959 | −23.446 | 1.00 | 81.31 |
| ATOM | 4218 | C | MSE | 2149 | 29.613 | 20.070 | −20.303 | 1.00 | 71.30 |
| ATOM | 4219 | O | MSE | 2149 | 28.993 | 19.709 | −19.300 | 1.00 | 71.53 |
| ATOM | 4220 | N | MSE | 2149 | 29.736 | 18.408 | −22.143 | 1.00 | 72.18 |
| ATOM | 4221 | CA | MSE | 2149 | 29.125 | 19.699 | −21.714 | 1.00 | 72.61 |
| ATOM | 4222 | N | PRO | 2150 | 30.731 | 20.811 | −20.217 | 1.00 | 69.63 |
| ATOM | 4223 | CD | PRO | 2150 | 31.405 | 21.382 | −21.394 | 1.00 | 69.49 |
| ATOM | 4224 | CA | PRO | 2150 | 31.375 | 21.273 | −18.977 | 1.00 | 68.16 |
| ATOM | 4225 | CB | PRO | 2150 | 32.479 | 22.200 | −19.479 | 1.00 | 68.32 |
| ATOM | 4226 | CG | PRO | 2150 | 32.777 | 21.684 | −20.850 | 1.00 | 69.55 |
| ATOM | 4227 | C | PRO | 2150 | 30.460 | 22.016 | −18.014 | 1.00 | 66.65 |
| ATOM | 4228 | O | PRO | 2150 | 29.831 | 23.001 | −18.395 | 1.00 | 66.56 |
| ATOM | 4229 | N | VAL | 2151 | 30.405 | 21.556 | −16.766 | 1.00 | 64.70 |
| ATOM | 4230 | CA | VAL | 2151 | 29.587 | 22.206 | −15.751 | 1.00 | 62.62 |
| ATOM | 4231 | CB | VAL | 2151 | 28.297 | 21.438 | −15.496 | 1.00 | 62.77 |
| ATOM | 4232 | CG1 | VAL | 2151 | 27.428 | 22.208 | −14.511 | 1.00 | 62.32 |
| ATOM | 4233 | CG2 | VAL | 2151 | 27.568 | 21.222 | −16.807 | 1.00 | 62.65 |
| ATOM | 4234 | C | VAL | 2151 | 30.342 | 22.337 | −14.433 | 1.00 | 61.28 |
| ATOM | 4235 | O | VAL | 2151 | 30.806 | 21.345 | −13.870 | 1.00 | 61.55 |
| ATOM | 4236 | N | ALA | 2152 | 30.476 | 23.571 | −13.955 | 1.00 | 59.27 |
| ATOM | 4237 | CA | ALA | 2152 | 31.163 | 23.833 | −12.696 | 1.00 | 57.33 |
| ATOM | 4238 | CB | ALA | 2152 | 31.343 | 25.330 | −12.496 | 1.00 | 56.70 |
| ATOM | 4239 | C | ALA | 2152 | 30.321 | 23.242 | −11.562 | 1.00 | 56.10 |
| ATOM | 4240 | O | ALA | 2152 | 29.087 | 23.304 | −11.594 | 1.00 | 55.91 |
| ATOM | 4241 | N | PRO | 2153 | 30.980 | 22.678 | −10.535 | 1.00 | 54.52 |
| ATOM | 4242 | CD | PRO | 2153 | 32.424 | 22.823 | −10.287 | 1.00 | 53.98 |
| ATOM | 4243 | CA | PRO | 2153 | 30.318 | 22.060 | −9.379 | 1.00 | 52.93 |
| ATOM | 4244 | CB | PRO | 2153 | 31.478 | 21.806 | −8.413 | 1.00 | 53.35 |
| ATOM | 4245 | CG | PRO | 2153 | 32.480 | 22.853 | −8.790 | 1.00 | 53.56 |
| ATOM | 4246 | C | PRO | 2153 | 29.186 | 22.853 | −8.739 | 1.00 | 51.17 |
| ATOM | 4247 | O | PRO | 2153 | 29.262 | 24.065 | −8.591 | 1.00 | 51.17 |
| ATOM | 4248 | N | TYR | 2154 | 28.132 | 22.143 | −8.365 | 1.00 | 49.67 |
| ATOM | 4249 | CA | TYR | 2154 | 26.966 | 22.751 | −7.733 | 1.00 | 48.69 |
| ATOM | 4250 | CB | TYR | 2154 | 25.940 | 23.170 | −8.799 | 1.00 | 47.37 |
| ATOM | 4251 | CG | TYR | 2154 | 25.395 | 22.015 | −9.622 | 1.00 | 46.49 |
| ATOM | 4252 | CD1 | TYR | 2154 | 26.202 | 21.343 | −10.548 | 1.00 | 45.55 |
| ATOM | 4253 | CE1 | TYR | 2154 | 25.721 | 20.248 | −11.272 | 1.00 | 45.14 |
| ATOM | 4254 | CD2 | TYR | 2154 | 24.087 | 21.564 | −9.445 | 1.00 | 46.44 |
| ATOM | 4255 | CE2 | TYR | 2154 | 23.595 | 20.466 | −10.165 | 1.00 | 46.07 |
| ATOM | 4256 | CZ | TYR | 2154 | 24.419 | 19.813 | −11.072 | 1.00 | 45.55 |
| ATOM | 4257 | OH | TYR | 2154 | 23.949 | 18.713 | −11.748 | 1.00 | 44.58 |
| ATOM | 4258 | C | TYR | 2154 | 26.321 | 21.749 | −6.765 | 1.00 | 48.29 |
| ATOM | 4259 | O | TYR | 2154 | 26.421 | 20.537 | −6.950 | 1.00 | 47.58 |
| ATOM | 4260 | N | TRP | 2155 | 25.659 | 22.262 | −5.736 | 1.00 | 47.79 |
| ATOM | 4261 | CA | TRP | 2155 | 24.997 | 21.413 | −4.761 | 1.00 | 47.39 |
| ATOM | 4262 | CB | TRP | 2155 | 24.578 | 22.235 | −3.541 | 1.00 | 45.56 |
| ATOM | 4263 | CG | TRP | 2155 | 25.696 | 22.971 | −2.860 | 1.00 | 43.22 |
| ATOM | 4264 | CD2 | TRP | 2155 | 26.939 | 22.425 | −2.403 | 1.00 | 41.64 |
| ATOM | 4265 | CE2 | TRP | 2155 | 27.659 | 23.476 | −1.801 | 1.00 | 41.47 |
| ATOM | 4266 | CE3 | TRP | 2155 | 27.515 | 21.150 | −2.444 | 1.00 | 41.19 |
| ATOM | 4267 | CD1 | TRP | 2155 | 25.713 | 24.291 | −2.524 | 1.00 | 42.25 |
| ATOM | 4268 | NE1 | TRP | 2155 | 26.886 | 24.604 | −1.889 | 1.00 | 41.44 |
| ATOM | 4269 | CZ2 | TRP | 2155 | 28.926 | 23.294 | −1.242 | 1.00 | 41.05 |
| ATOM | 4270 | CZ3 | TRP | 2155 | 28.778 | 20.970 | −1.889 | 1.00 | 40.72 |
| ATOM | 4271 | CH2 | TRP | 2155 | 29.467 | 22.038 | −1.297 | 1.00 | 40.52 |
| ATOM | 4272 | C | TRP | 2155 | 23.758 | 20.834 | −5.404 | 1.00 | 48.35 |
| ATOM | 4273 | O | TRP | 2155 | 23.023 | 21.546 | −6.086 | 1.00 | 49.28 |
| ATOM | 4274 | N | THR | 2156 | 23.520 | 19.550 | −5.176 | 1.00 | 49.34 |
| ATOM | 4275 | CA | THR | 2156 | 22.353 | 18.871 | −5.724 | 1.00 | 50.45 |
| ATOM | 4276 | CB | THR | 2156 | 22.710 | 17.437 | −6.115 | 1.00 | 50.33 |
| ATOM | 4277 | OG1 | THR | 2156 | 23.528 | 16.860 | −5.087 | 1.00 | 49.82 |
| ATOM | 4278 | CG2 | THR | 2156 | 23.464 | 17.413 | −7.438 | 1.00 | 50.54 |
| ATOM | 4279 | C | THR | 2156 | 21.207 | 18.823 | −4.716 | 1.00 | 51.29 |
| ATOM | 4280 | O | THR | 2156 | 20.043 | 18.726 | −5.098 | 1.00 | 51.55 |
| ATOM | 4281 | N | SER | 2157 | 21.546 | 18.882 | −3.430 | 1.00 | 52.50 |
| ATOM | 4282 | CA | SER | 2157 | 20.551 | 18.838 | −2.356 | 1.00 | 53.40 |
| ATOM | 4283 | CB | SER | 2157 | 20.566 | 17.478 | −1.648 | 1.00 | 53.00 |
| ATOM | 4284 | OG | SER | 2157 | 20.499 | 16.397 | −2.553 | 1.00 | 53.76 |
| ATOM | 4285 | C | SER | 2157 | 20.843 | 19.899 | −1.304 | 1.00 | 54.11 |
| ATOM | 4286 | O | SER | 2157 | 20.993 | 19.573 | −0.128 | 1.00 | 54.08 |
| ATOM | 4287 | N | PRO | 2158 | 20.916 | 21.179 | −1.699 | 1.00 | 54.95 |
| ATOM | 4288 | CD | PRO | 2158 | 20.491 | 21.757 | −2.984 | 1.00 | 54.58 |
| ATOM | 4289 | CA | PRO | 2158 | 21.199 | 22.239 | −0.726 | 1.00 | 55.65 |
| ATOM | 4290 | CB | PRO | 2158 | 21.066 | 23.508 | −1.560 | 1.00 | 55.16 |
| ATOM | 4291 | CG | PRO | 2158 | 20.045 | 23.130 | −2.575 | 1.00 | 54.58 |
| ATOM | 4292 | C | PRO | 2158 | 20.232 | 22.201 | 0.453 | 1.00 | 56.63 |
| ATOM | 4293 | O | PRO | 2158 | 20.530 | 22.699 | 1.539 | 1.00 | 56.40 |
| ATOM | 4294 | N | GLU | 2159 | 19.074 | 21.592 | 0.224 | 1.00 | 57.62 |
| ATOM | 4295 | CA | GLU | 2159 | 18.055 | 21.462 | 1.251 | 1.00 | 58.67 |
| ATOM | 4296 | CB | GLU | 2159 | 16.826 | 20.725 | 0.708 | 1.00 | 59.72 |
| ATOM | 4297 | CG | GLU | 2159 | 16.305 | 21.217 | −0.633 | 1.00 | 61.86 |
| ATOM | 4298 | CD | GLU | 2159 | 17.039 | 20.598 | −1.814 | 1.00 | 62.80 |
| ATOM | 4299 | OE1 | GLU | 2159 | 17.689 | 19.546 | −1.623 | 1.00 | 63.02 |
| ATOM | 4300 | OE2 | GLU | 2159 | 16.947 | 21.153 | −2.936 | 1.00 | 63.64 |
| ATOM | 4301 | C | GLU | 2159 | 18.588 | 20.678 | 2.444 | 1.00 | 58.46 |
| ATOM | 4302 | O | GLU | 2159 | 18.370 | 21.064 | 3.592 | 1.00 | 58.79 |
| ATOM | 4303 | N | LYS | 2160 | 19.272 | 19.568 | 2.180 | 1.00 | 58.04 |
| ATOM | 4304 | CA | LYS | 2160 | 19.788 | 18.766 | 3.278 | 1.00 | 57.31 |
| ATOM | 4305 | CB | LYS | 2160 | 19.808 | 17.273 | 2.919 | 1.00 | 57.70 |
| ATOM | 4306 | CG | LYS | 2160 | 20.735 | 16.867 | 1.796 | 1.00 | 59.16 |
| ATOM | 4307 | CD | LYS | 2160 | 20.837 | 15.340 | 1.691 | 1.00 | 59.95 |
| ATOM | 4308 | CE | LYS | 2160 | 19.486 | 14.681 | 1.406 | 1.00 | 60.48 |
| ATOM | 4309 | NZ | LYS | 2160 | 19.624 | 13.203 | 1.191 | 1.00 | 61.55 |
| ATOM | 4310 | C | LYS | 2160 | 21.147 | 19.221 | 3.807 | 1.00 | 56.62 |
| ATOM | 4311 | O | LYS | 2160 | 21.859 | 18.450 | 4.452 | 1.00 | 56.95 |
| ATOM | 4312 | N | MSE | 2161 | 21.493 | 20.478 | 3.549 | 1.00 | 55.45 |
| ATOM | 4313 | CA | MSE | 2161 | 22.749 | 21.053 | 4.032 | 1.00 | 54.44 |
| ATOM | 4314 | CB | MSE | 2161 | 23.607 | 21.551 | 2.858 | 1.00 | 52.60 |
| ATOM | 4315 | CG | MSE | 2161 | 24.164 | 20.457 | 1.951 | 1.00 | 50.49 |
| ATOM | 4316 | SE | MSE | 2161 | 24.973 | 21.102 | 0.448 | 1.00 | 47.99 |
| ATOM | 4317 | CE | MSE | 2161 | 26.488 | 21.782 | 1.124 | 1.00 | 48.51 |
| ATOM | 4318 | C | MSE | 2161 | 22.397 | 22.219 | 4.960 | 1.00 | 54.88 |
| ATOM | 4319 | O | MSE | 2161 | 23.242 | 23.047 | 5.309 | 1.00 | 55.06 |
| ATOM | 4320 | N | GLU | 2162 | 21.129 | 22.260 | 5.354 | 1.00 | 55.27 |
| ATOM | 4321 | CA | GLU | 2162 | 20.578 | 23.293 | 6.227 | 1.00 | 55.39 |
| ATOM | 4322 | CB | GLU | 2162 | 19.073 | 23.027 | 6.388 | 1.00 | 57.63 |
| ATOM | 4323 | CG | GLU | 2162 | 18.202 | 24.264 | 6.460 | 1.00 | 60.68 |
| ATOM | 4324 | CD | GLU | 2162 | 18.509 | 25.249 | 5.350 | 1.00 | 62.78 |
| ATOM | 4325 | OE1 | GLU | 2162 | 19.454 | 26.064 | 5.525 | 1.00 | 64.01 |
| ATOM | 4326 | OE2 | GLU | 2162 | 17.814 | 25.195 | 4.305 | 1.00 | 63.10 |
| ATOM | 4327 | C | GLU | 2162 | 21.257 | 23.333 | 7.604 | 1.00 | 53.75 |
| ATOM | 4328 | O | GLU | 2162 | 21.809 | 24.356 | 8.010 | 1.00 | 53.26 |
| ATOM | 4329 | N | LYS | 2163 | 21.197 | 22.202 | 8.301 | 1.00 | 51.69 |
| ATOM | 4330 | CA | LYS | 2163 | 21.762 | 22.017 | 9.634 | 1.00 | 49.98 |
| ATOM | 4331 | CB | LYS | 2163 | 21.379 | 20.609 | 10.098 | 1.00 | 49.52 |
| ATOM | 4332 | CG | LYS | 2163 | 22.052 | 20.056 | 11.335 | 1.00 | 48.96 |
| ATOM | 4333 | CD | LYS | 2163 | 21.567 | 18.624 | 11.515 | 1.00 | 48.61 |
| ATOM | 4334 | CE | LYS | 2163 | 22.183 | 17.932 | 12.706 | 1.00 | 49.16 |
| ATOM | 4335 | NZ | LYS | 2163 | 21.414 | 16.697 | 13.049 | 1.00 | 49.79 |
| ATOM | 4336 | C | LYS | 2163 | 23.280 | 22.210 | 9.676 | 1.00 | 48.78 |
| ATOM | 4337 | O | LYS | 2163 | 24.031 | 21.313 | 9.316 | 1.00 | 48.57 |
| ATOM | 4338 | N | LYS | 2164 | 23.734 | 23.376 | 10.123 | 1.00 | 47.52 |
| ATOM | 4339 | CA | LYS | 2164 | 25.163 | 23.619 | 10.185 | 1.00 | 46.14 |
| ATOM | 4340 | CB | LYS | 2164 | 25.463 | 25.095 | 9.915 | 1.00 | 47.42 |
| ATOM | 4341 | CG | LYS | 2164 | 25.304 | 25.459 | 8.428 | 1.00 | 49.19 |
| ATOM | 4342 | CD | LYS | 2164 | 26.100 | 26.704 | 8.055 | 1.00 | 51.03 |
| ATOM | 4343 | CE | LYS | 2164 | 27.584 | 26.543 | 8.405 | 1.00 | 51.49 |
| ATOM | 4344 | NZ | LYS | 2164 | 28.423 | 27.692 | 7.949 | 1.00 | 51.95 |
| ATOM | 4345 | C | LYS | 2164 | 25.820 | 23.135 | 11.476 | 1.00 | 44.65 |
| ATOM | 4346 | O | LYS | 2164 | 26.934 | 22.626 | 11.436 | 1.00 | 44.68 |
| ATOM | 4347 | N | LEU | 2165 | 25.140 | 23.273 | 12.615 | 1.00 | 42.19 |
| ATOM | 4348 | CA | LEU | 2165 | 25.705 | 22.791 | 13.867 | 1.00 | 39.70 |
| ATOM | 4349 | CB | LEU | 2165 | 25.338 | 23.700 | 15.042 | 1.00 | 38.84 |
| ATOM | 4350 | CG | LEU | 2165 | 25.670 | 23.090 | 16.416 | 1.00 | 37.93 |
| ATOM | 4351 | CD1 | LEU | 2165 | 27.178 | 22.986 | 16.591 | 1.00 | 37.10 |
| ATOM | 4352 | CD2 | LEU | 2165 | 25.053 | 23.921 | 17.529 | 1.00 | 37.29 |
| ATOM | 4353 | C | LEU | 2165 | 25.219 | 21.385 | 14.177 | 1.00 | 38.63 |
| ATOM | 4354 | O | LEU | 2165 | 24.025 | 21.130 | 14.229 | 1.00 | 38.28 |
| ATOM | 4355 | N | HIS | 2166 | 26.156 | 20.468 | 14.368 | 1.00 | 37.41 |
| ATOM | 4356 | CA | HIS | 2166 | 25.813 | 19.096 | 14.714 | 1.00 | 36.06 |
| ATOM | 4357 | CB | HIS | 2166 | 26.517 | 18.067 | 13.813 | 1.00 | 36.60 |
| ATOM | 4358 | CG | HIS | 2166 | 25.967 | 17.966 | 12.419 | 1.00 | 38.11 |
| ATOM | 4359 | CD2 | HIS | 2166 | 25.856 | 18.885 | 11.426 | 1.00 | 38.56 |
| ATOM | 4360 | ND1 | HIS | 2166 | 25.518 | 16.774 | 11.883 | 1.00 | 38.28 |
| ATOM | 4361 | CE1 | HIS | 2166 | 25.154 | 16.964 | 10.625 | 1.00 | 37.67 |
| ATOM | 4362 | NE2 | HIS | 2166 | 25.348 | 18.236 | 10.323 | 1.00 | 37.80 |
| ATOM | 4363 | C | HIS | 2166 | 26.310 | 18.904 | 16.139 | 1.00 | 35.15 |
| ATOM | 4364 | O | HIS | 2166 | 27.520 | 18.896 | 16.392 | 1.00 | 35.14 |
| ATOM | 4365 | N | ALA | 2167 | 25.378 | 18.769 | 17.071 | 1.00 | 33.33 |
| ATOM | 4366 | CA | ALA | 2167 | 25.748 | 18.559 | 18.455 | 1.00 | 31.78 |
| ATOM | 4367 | CB | ALA | 2167 | 25.056 | 19.591 | 19.345 | 1.00 | 31.04 |
| ATOM | 4368 | C | ALA | 2167 | 25.308 | 17.144 | 18.809 | 1.00 | 30.13 |
| ATOM | 4369 | O | ALA | 2167 | 24.183 | 16.744 | 18.531 | 1.00 | 30.31 |
| ATOM | 4370 | N | VAL | 2168 | 26.206 | 16.375 | 19.398 | 1.00 | 29.06 |
| ATOM | 4371 | CA | VAL | 2168 | 25.875 | 15.009 | 19.768 | 1.00 | 27.84 |
| ATOM | 4372 | CB | VAL | 2168 | 26.288 | 14.010 | 18.674 | 1.00 | 28.12 |
| ATOM | 4373 | CG1 | VAL | 2168 | 25.514 | 14.265 | 17.401 | 1.00 | 28.23 |
| ATOM | 4374 | CG2 | VAL | 2168 | 27.782 | 14.119 | 18.429 | 1.00 | 27.63 |
| ATOM | 4375 | C | VAL | 2168 | 26.601 | 14.580 | 21.021 | 1.00 | 26.89 |
| ATOM | 4376 | O | VAL | 2168 | 27.626 | 15.155 | 21.389 | 1.00 | 27.64 |
| ATOM | 4377 | N | PRO | 2169 | 26.071 | 13.563 | 21.697 | 1.00 | 26.04 |
| ATOM | 4378 | CD | PRO | 2169 | 24.781 | 12.890 | 21.451 | 1.00 | 26.22 |
| ATOM | 4379 | CA | PRO | 2169 | 26.712 | 13.067 | 22.911 | 1.00 | 26.17 |
| ATOM | 4380 | CB | PRO | 2169 | 25.624 | 12.191 | 23.527 | 1.00 | 25.80 |
| ATOM | 4381 | CG | PRO | 2169 | 24.875 | 11.675 | 22.324 | 1.00 | 25.68 |
| ATOM | 4382 | C | PRO | 2169 | 27.924 | 12.272 | 22.435 | 1.00 | 26.20 |
| ATOM | 4383 | O | PRO | 2169 | 27.969 | 11.856 | 21.283 | 1.00 | 26.80 |
| ATOM | 4384 | N | ALA | 2170 | 28.904 | 12.074 | 23.305 | 1.00 | 26.35 |
| ATOM | 4385 | CA | ALA | 2170 | 30.088 | 11.310 | 22.953 | 1.00 | 26.76 |
| ATOM | 4386 | CB | ALA | 2170 | 31.017 | 11.271 | 24.140 | 1.00 | 25.11 |
| ATOM | 4387 | C | ALA | 2170 | 29.714 | 9.888 | 22.532 | 1.00 | 26.88 |
| ATOM | 4388 | O | ALA | 2170 | 28.696 | 9.354 | 22.965 | 1.00 | 27.39 |
| ATOM | 4389 | N | ALA | 2171 | 30.530 | 9.294 | 21.667 | 1.00 | 27.36 |
| ATOM | 4390 | CA | ALA | 2171 | 30.336 | 7.912 | 21.207 | 1.00 | 27.41 |
| ATOM | 4391 | CB | ALA | 2171 | 29.865 | 7.022 | 22.380 | 1.00 | 25.72 |
| ATOM | 4392 | C | ALA | 2171 | 29.401 | 7.747 | 20.022 | 1.00 | 27.07 |
| ATOM | 4393 | O | ALA | 2171 | 29.355 | 6.678 | 19.415 | 1.00 | 28.23 |
| ATOM | 4394 | N | LYS | 2172 | 28.654 | 8.791 | 19.685 | 1.00 | 26.94 |
| ATOM | 4395 | CA | LYS | 2172 | 27.742 | 8.679 | 18.560 | 1.00 | 26.72 |
| ATOM | 4396 | CB | LYS | 2172 | 26.683 | 9.778 | 18.592 | 1.00 | 26.60 |
| ATOM | 4397 | CG | LYS | 2172 | 25.553 | 9.474 | 17.623 | 1.00 | 25.72 |
| ATOM | 4398 | CD | LYS | 2172 | 24.290 | 10.258 | 17.874 | 1.00 | 25.26 |
| ATOM | 4399 | CE | LYS | 2172 | 23.223 | 9.772 | 16.914 | 1.00 | 25.55 |
| ATOM | 4400 | NZ | LYS | 2172 | 23.772 | 9.737 | 15.524 | 1.00 | 25.48 |
| ATOM | 4401 | C | LYS | 2172 | 28.482 | 8.732 | 17.228 | 1.00 | 27.01 |
| ATOM | 4402 | O | LYS | 2172 | 29.621 | 9.203 | 17.156 | 1.00 | 26.80 |
| ATOM | 4403 | N | THR | 2173 | 27.838 | 8.220 | 16.183 | 1.00 | 26.96 |
| ATOM | 4404 | CA | THR | 2173 | 28.397 | 8.234 | 14.833 | 1.00 | 27.33 |
| ATOM | 4405 | CB | THR | 2173 | 27.847 | 7.067 | 13.960 | 1.00 | 27.16 |
| ATOM | 4406 | OG1 | THR | 2173 | 28.414 | 5.831 | 14.401 | 1.00 | 28.92 |
| ATOM | 4407 | CG2 | THR | 2173 | 28.201 | 7.260 | 12.479 | 1.00 | 26.33 |
| ATOM | 4408 | C | THR | 2173 | 27.951 | 9.540 | 14.200 | 1.00 | 27.90 |
| ATOM | 4409 | O | THR | 2173 | 26.788 | 9.907 | 14.308 | 1.00 | 28.78 |
| ATOM | 4410 | N | VAL | 2174 | 28.870 | 10.241 | 13.547 | 1.00 | 28.51 |
| ATOM | 4411 | CA | VAL | 2174 | 28.543 | 11.509 | 12.889 | 1.00 | 29.09 |
| ATOM | 4412 | CB | VAL | 2174 | 29.417 | 12.698 | 13.419 | 1.00 | 28.76 |
| ATOM | 4413 | CG1 | VAL | 2174 | 29.095 | 13.973 | 12.645 | 1.00 | 27.33 |
| ATOM | 4414 | CG2 | VAL | 2174 | 29.180 | 12.908 | 14.909 | 1.00 | 27.78 |
| ATOM | 4415 | C | VAL | 2174 | 28.781 | 11.397 | 11.394 | 1.00 | 30.09 |
| ATOM | 4416 | O | VAL | 2174 | 29.792 | 10.851 | 10.949 | 1.00 | 30.49 |
| ATOM | 4417 | N | LYS | 2175 | 27.852 | 11.932 | 10.617 | 1.00 | 31.14 |
| ATOM | 4418 | CA | LYS | 2175 | 27.975 | 11.881 | 9.175 | 1.00 | 32.35 |
| ATOM | 4419 | CB | LYS | 2175 | 27.024 | 10.822 | 8.613 | 1.00 | 32.05 |
| ATOM | 4420 | CG | LYS | 2175 | 26.899 | 10.844 | 7.099 | 1.00 | 34.13 |
| ATOM | 4421 | CD | LYS | 2175 | 26.295 | 9.545 | 6.572 | 1.00 | 35.66 |
| ATOM | 4422 | CE | LYS | 2175 | 26.268 | 9.531 | 5.055 | 1.00 | 37.66 |
| ATOM | 4423 | NZ | LYS | 2175 | 25.865 | 8.206 | 4.490 | 1.00 | 39.96 |
| ATOM | 4424 | C | LYS | 2175 | 27.689 | 13.236 | 8.530 | 1.00 | 32.92 |
| ATOM | 4425 | O | LYS | 2175 | 26.611 | 13.802 | 8.713 | 1.00 | 32.90 |
| ATOM | 4426 | N | PHE | 2176 | 28.668 | 13.755 | 7.791 | 1.00 | 33.16 |
| ATOM | 4427 | CA | PHE | 2176 | 28.514 | 15.026 | 7.090 | 1.00 | 33.55 |
| ATOM | 4428 | CB | PHE | 2176 | 29.691 | 15.970 | 7.347 | 1.00 | 33.25 |
| ATOM | 4429 | CG | PHE | 2176 | 29.859 | 16.378 | 8.786 | 1.00 | 34.34 |
| ATOM | 4430 | CD1 | PHE | 2176 | 28.774 | 16.830 | 9.534 | 1.00 | 33.63 |
| ATOM | 4431 | CD2 | PHE | 2176 | 31.117 | 16.338 | 9.389 | 1.00 | 33.78 |
| ATOM | 4432 | CE1 | PHE | 2176 | 28.942 | 17.234 | 10.852 | 1.00 | 33.41 |
| ATOM | 4433 | CE2 | PHE | 2176 | 31.288 | 16.743 | 10.712 | 1.00 | 33.39 |
| ATOM | 4434 | CZ | PHE | 2176 | 30.198 | 17.190 | 11.440 | 1.00 | 32.98 |
| ATOM | 4435 | C | PHE | 2176 | 28.469 | 14.736 | 5.601 | 1.00 | 34.40 |
| ATOM | 4436 | O | PHE | 2176 | 29.196 | 13.880 | 5.104 | 1.00 | 35.38 |
| ATOM | 4437 | N | LYS | 2177 | 27.629 | 15.460 | 4.881 | 1.00 | 35.14 |
| ATOM | 4438 | CA | LYS | 2177 | 27.521 | 15.248 | 3.455 | 1.00 | 36.24 |
| ATOM | 4439 | CB | LYS | 2177 | 26.342 | 14.322 | 3.177 | 1.00 | 35.89 |
| ATOM | 4440 | CG | LYS | 2177 | 25.022 | 14.868 | 3.667 | 1.00 | 36.13 |
| ATOM | 4441 | CD | LYS | 2177 | 24.068 | 13.734 | 3.991 | 1.00 | 38.00 |
| ATOM | 4442 | CE | LYS | 2177 | 22.963 | 14.189 | 4.941 | 1.00 | 38.26 |
| ATOM | 4443 | NZ | LYS | 2177 | 22.188 | 13.028 | 5.472 | 1.00 | 39.47 |
| ATOM | 4444 | C | LYS | 2177 | 27.361 | 16.546 | 2.670 | 1.00 | 37.27 |
| ATOM | 4445 | O | LYS | 2177 | 26.779 | 17.516 | 3.146 | 1.00 | 37.30 |
| ATOM | 4446 | N | CYS | 2178 | 27.888 | 16.548 | 1.455 | 1.00 | 38.80 |
| ATOM | 4447 | CA | CYS | 2178 | 27.798 | 17.702 | 0.569 | 1.00 | 40.55 |
| ATOM | 4448 | CB | CYS | 2178 | 29.128 | 18.449 | 0.536 | 1.00 | 41.15 |
| ATOM | 4449 | SG | CYS | 2178 | 29.573 | 19.141 | 2.127 | 1.00 | 42.48 |
| ATOM | 4450 | C | CYS | 2178 | 27.461 | 17.209 | −0.825 | 1.00 | 40.91 |
| ATOM | 4451 | O | CYS | 2178 | 28.284 | 17.281 | −1.732 | 1.00 | 40.87 |
| ATOM | 4452 | N | PRO | 2179 | 26.240 | 16.692 | −1.010 | 1.00 | 41.63 |
| ATOM | 4453 | CD | PRO | 2179 | 25.160 | 16.546 | −0.021 | 1.00 | 41.07 |
| ATOM | 4454 | CA | PRO | 2179 | 25.817 | 16.185 | −2.312 | 1.00 | 42.34 |
| ATOM | 4455 | CB | PRO | 2179 | 24.363 | 15.794 | −2.069 | 1.00 | 41.77 |
| ATOM | 4456 | CG | PRO | 2179 | 24.347 | 15.446 | −0.624 | 1.00 | 41.36 |
| ATOM | 4457 | C | PRO | 2179 | 25.957 | 17.253 | −3.383 | 1.00 | 43.74 |
| ATOM | 4458 | O | PRO | 2179 | 25.379 | 18.341 | −3.279 | 1.00 | 44.41 |
| ATOM | 4459 | N | SER | 2180 | 26.742 | 16.949 | −4.407 | 1.00 | 45.16 |
| ATOM | 4460 | CA | SER | 2180 | 26.937 | 17.892 | −5.492 | 1.00 | 46.33 |
| ATOM | 4461 | CB | SER | 2180 | 28.117 | 18.829 | −5.184 | 1.00 | 46.99 |
| ATOM | 4462 | OG | SER | 2180 | 29.325 | 18.121 | −4.972 | 1.00 | 47.89 |
| ATOM | 4463 | C | SER | 2180 | 27.166 | 17.167 | −6.802 | 1.00 | 46.69 |
| ATOM | 4464 | O | SER | 2180 | 27.104 | 15.940 | −6.872 | 1.00 | 46.56 |
| ATOM | 4465 | N | SER | 2181 | 27.400 | 17.938 | −7.854 | 1.00 | 47.95 |
| ATOM | 4466 | CA | SER | 2181 | 27.662 | 17.353 | −9.159 | 1.00 | 48.36 |
| ATOM | 4467 | CB | SER | 2181 | 26.369 | 16.851 | −9.811 | 1.00 | 48.23 |
| ATOM | 4468 | OG | SER | 2181 | 26.668 | 15.915 | −10.837 | 1.00 | 48.14 |
| ATOM | 4469 | C | SER | 2181 | 28.353 | 18.366 | −10.056 | 1.00 | 48.58 |
| ATOM | 4470 | O | SER | 2181 | 28.694 | 19.470 | −9.622 | 1.00 | 48.69 |
| ATOM | 4471 | N | GLY | 2182 | 28.562 | 17.974 | −11.306 | 1.00 | 48.78 |
| ATOM | 4472 | CA | GLY | 2182 | 29.230 | 18.831 | −12.259 | 1.00 | 48.82 |
| ATOM | 4473 | C | GLY | 2182 | 30.009 | 17.951 | −13.207 | 1.00 | 49.05 |
| ATOM | 4474 | O | GLY | 2182 | 30.203 | 16.762 | −12.946 | 1.00 | 49.04 |
| ATOM | 4475 | N | THR | 2183 | 30.457 | 18.528 | −14.312 | 1.00 | 49.35 |
| ATOM | 4476 | CA | THR | 2183 | 31.214 | 17.768 | −15.291 | 1.00 | 49.45 |
| ATOM | 4477 | CB | THR | 2183 | 30.282 | 17.325 | −16.454 | 1.00 | 50.04 |
| ATOM | 4478 | OG1 | THR | 2183 | 29.456 | 18.421 | −16.858 | 1.00 | 50.89 |
| ATOM | 4479 | CG2 | THR | 2183 | 29.360 | 16.189 | −15.988 | 1.00 | 50.87 |
| ATOM | 4480 | C | THR | 2183 | 32.419 | 18.557 | −15.801 | 1.00 | 48.88 |
| ATOM | 4481 | O | THR | 2183 | 32.303 | 19.720 | −16.180 | 1.00 | 48.04 |
| ATOM | 4482 | N | PRO | 2184 | 33.606 | 17.937 | −15.771 | 1.00 | 48.98 |
| ATOM | 4483 | CD | PRO | 2184 | 34.893 | 18.577 | −16.092 | 1.00 | 48.47 |
| ATOM | 4484 | CA | PRO | 2184 | 33.803 | 16.565 | −15.283 | 1.00 | 49.10 |
| ATOM | 4485 | CB | PRO | 2184 | 35.287 | 16.326 | −15.551 | 1.00 | 49.11 |
| ATOM | 4486 | CG | PRO | 2184 | 35.878 | 17.705 | −15.359 | 1.00 | 49.00 |
| ATOM | 4487 | C | PRO | 2184 | 33.429 | 16.415 | −13.799 | 1.00 | 48.91 |
| ATOM | 4488 | O | PRO | 2184 | 33.314 | 17.413 | −13.075 | 1.00 | 49.04 |
| ATOM | 4489 | N | GLN | 2185 | 33.229 | 15.177 | −13.354 | 1.00 | 48.65 |
| ATOM | 4490 | CA | GLN | 2185 | 32.863 | 14.923 | −11.967 | 1.00 | 47.59 |
| ATOM | 4491 | CB | GLN | 2185 | 32.781 | 13.423 | −11.700 | 1.00 | 47.63 |
| ATOM | 4492 | CG | GLN | 2185 | 31.829 | 13.047 | −10.573 | 1.00 | 47.53 |
| ATOM | 4493 | CD | GLN | 2185 | 30.363 | 13.301 | −10.922 | 1.00 | 47.56 |
| ATOM | 4494 | OE1 | GLN | 2185 | 29.481 | 13.137 | −10.084 | 1.00 | 47.06 |
| ATOM | 4495 | NE2 | GLN | 2185 | 30.103 | 13.695 | −12.164 | 1.00 | 47.86 |
| ATOM | 4496 | C | GLN | 2185 | 33.894 | 15.562 | −11.053 | 1.00 | 47.39 |
| ATOM | 4497 | O | GLN | 2185 | 35.092 | 15.325 | −11.189 | 1.00 | 47.73 |
| ATOM | 4498 | N | PRO | 2186 | 33.437 | 16.402 | −10.113 | 1.00 | 47.07 |
| ATOM | 4499 | CD | PRO | 2186 | 32.044 | 16.855 | −9.951 | 1.00 | 47.24 |
| ATOM | 4500 | CA | PRO | 2186 | 34.320 | 17.091 | −9.169 | 1.00 | 46.33 |
| ATOM | 4501 | CB | PRO | 2186 | 33.432 | 18.218 | −8.646 | 1.00 | 46.67 |
| ATOM | 4502 | CG | PRO | 2186 | 32.094 | 17.583 | −8.629 | 1.00 | 46.84 |
| ATOM | 4503 | C | PRO | 2186 | 34.847 | 16.193 | −8.055 | 1.00 | 45.40 |
| ATOM | 4504 | O | PRO | 2186 | 34.193 | 15.227 | −7.673 | 1.00 | 44.57 |
| ATOM | 4505 | N | THR | 2187 | 36.039 | 16.511 | −7.549 | 1.00 | 45.02 |
| ATOM | 4506 | CA | THR | 2187 | 36.635 | 15.725 | −6.468 | 1.00 | 44.28 |
| ATOM | 4507 | CB | THR | 2187 | 38.176 | 15.832 | −6.430 | 1.00 | 44.83 |
| ATOM | 4508 | OG1 | THR | 2187 | 38.553 | 17.074 | −5.814 | 1.00 | 45.38 |
| ATOM | 4509 | CG2 | THR | 2187 | 38.753 | 15.754 | −7.836 | 1.00 | 44.35 |
| ATOM | 4510 | C | THR | 2187 | 36.108 | 16.249 | −5.143 | 1.00 | 43.08 |
| ATOM | 4511 | O | THR | 2187 | 35.550 | 17.350 | −5.071 | 1.00 | 42.73 |
| ATOM | 4512 | N | LEU | 2188 | 36.300 | 15.461 | −4.092 | 1.00 | 42.20 |
| ATOM | 4513 | CA | LEU | 2188 | 35.827 | 15.835 | −2.770 | 1.00 | 40.81 |
| ATOM | 4514 | CB | LEU | 2188 | 34.551 | 15.050 | −2.445 | 1.00 | 41.49 |
| ATOM | 4515 | CG | LEU | 2188 | 33.661 | 15.325 | −1.219 | 1.00 | 41.58 |
| ATOM | 4516 | CD1 | LEU | 2188 | 33.495 | 14.025 | −0.441 | 1.00 | 41.25 |
| ATOM | 4517 | CD2 | LEU | 2188 | 34.240 | 16.413 | −0.337 | 1.00 | 42.14 |
| ATOM | 4518 | C | LEU | 2188 | 36.874 | 15.557 | −1.707 | 1.00 | 40.20 |
| ATOM | 4519 | O | LEU | 2188 | 37.314 | 14.417 | −1.542 | 1.00 | 40.39 |
| ATOM | 4520 | N | ARG | 2189 | 37.276 | 16.602 | −0.991 | 1.00 | 39.09 |
| ATOM | 4521 | CA | ARG | 2189 | 38.241 | 16.447 | 0.087 | 1.00 | 37.74 |
| ATOM | 4522 | CB | ARG | 2189 | 39.619 | 16.973 | −0.338 | 1.00 | 37.43 |
| ATOM | 4523 | CG | ARG | 2189 | 39.688 | 18.438 | −0.722 | 1.00 | 39.19 |
| ATOM | 4524 | CD | ARG | 2189 | 40.955 | 18.722 | −1.558 | 1.00 | 40.39 |
| ATOM | 4525 | NE | ARG | 2189 | 41.494 | 20.055 | −1.303 | 1.00 | 40.93 |
| ATOM | 4526 | CZ | ARG | 2189 | 42.104 | 20.402 | −0.169 | 1.00 | 41.92 |
| ATOM | 4527 | NH1 | ARG | 2189 | 42.261 | 19.509 | 0.813 | 1.00 | 41.88 |
| ATOM | 4528 | NH2 | ARG | 2189 | 42.539 | 21.648 | −0.001 | 1.00 | 42.68 |
| ATOM | 4529 | C | ARG | 2189 | 37.699 | 17.177 | 1.310 | 1.00 | 36.62 |
| ATOM | 4530 | O | ARG | 2189 | 36.937 | 18.137 | 1.178 | 1.00 | 36.82 |
| ATOM | 4531 | N | TRP | 2190 | 38.061 | 16.700 | 2.497 | 1.00 | 35.09 |
| ATOM | 4532 | CA | TRP | 2190 | 37.587 | 17.303 | 3.734 | 1.00 | 33.81 |
| ATOM | 4533 | CB | TRP | 2190 | 36.871 | 16.260 | 4.588 | 1.00 | 32.95 |
| ATOM | 4534 | CG | TRP | 2190 | 35.621 | 15.713 | 3.975 | 1.00 | 32.18 |
| ATOM | 4535 | CD2 | TRP | 2190 | 34.292 | 16.193 | 4.192 | 1.00 | 32.29 |
| ATOM | 4536 | CE2 | TRP | 2190 | 33.425 | 15.385 | 3.420 | 1.00 | 32.14 |
| ATOM | 4537 | CE3 | TRP | 2190 | 33.747 | 17.227 | 4.967 | 1.00 | 31.77 |
| ATOM | 4538 | CD1 | TRP | 2190 | 35.516 | 14.666 | 3.105 | 1.00 | 31.54 |
| ATOM | 4539 | NE1 | TRP | 2190 | 34.196 | 14.462 | 2.767 | 1.00 | 31.62 |
| ATOM | 4540 | CZ2 | TRP | 2190 | 32.038 | 15.580 | 3.403 | 1.00 | 32.74 |
| ATOM | 4541 | CZ3 | TRP | 2190 | 32.369 | 17.422 | 4.951 | 1.00 | 31.59 |
| ATOM | 4542 | CH2 | TRP | 2190 | 31.530 | 16.601 | 4.173 | 1.00 | 31.96 |
| ATOM | 4543 | C | TRP | 2190 | 38.692 | 17.929 | 4.560 | 1.00 | 33.87 |
| ATOM | 4544 | O | TRP | 2190 | 39.827 | 17.460 | 4.564 | 1.00 | 33.86 |
| ATOM | 4545 | N | LEU | 2191 | 38.351 | 18.997 | 5.265 | 1.00 | 33.88 |
| ATOM | 4546 | CA | LEU | 2191 | 39.308 | 19.674 | 6.116 | 1.00 | 33.78 |
| ATOM | 4547 | CB | LEU | 2191 | 39.545 | 21.105 | 5.629 | 1.00 | 33.96 |
| ATOM | 4548 | CG | LEU | 2191 | 40.125 | 21.333 | 4.228 | 1.00 | 35.06 |
| ATOM | 4549 | CD1 | LEU | 2191 | 40.425 | 22.824 | 4.063 | 1.00 | 35.02 |
| ATOM | 4550 | CD2 | LEU | 2191 | 41.403 | 20.508 | 4.022 | 1.00 | 34.14 |
| ATOM | 4551 | C | LEU | 2191 | 38.767 | 19.717 | 7.537 | 1.00 | 33.71 |
| ATOM | 4552 | O | LEU | 2191 | 37.558 | 19.694 | 7.754 | 1.00 | 33.45 |
| ATOM | 4553 | N | LYS | 2192 | 39.667 | 19.758 | 8.508 | 1.00 | 33.64 |
| ATOM | 4554 | CA | LYS | 2192 | 39.254 | 19.866 | 9.891 | 1.00 | 33.48 |
| ATOM | 4555 | CB | LYS | 2192 | 39.718 | 18.662 | 10.701 | 1.00 | 31.81 |
| ATOM | 4556 | CG | LYS | 2192 | 39.312 | 18.749 | 12.153 | 1.00 | 30.33 |
| ATOM | 4557 | CD | LYS | 2192 | 39.782 | 17.537 | 12.926 | 1.00 | 29.72 |
| ATOM | 4558 | CE | LYS | 2192 | 39.709 | 17.781 | 14.417 | 1.00 | 27.70 |
| ATOM | 4559 | NZ | LYS | 2192 | 40.189 | 16.594 | 15.165 | 1.00 | 28.37 |
| ATOM | 4560 | C | LYS | 2192 | 39.895 | 21.153 | 10.394 | 1.00 | 33.87 |
| ATOM | 4561 | O | LYS | 2192 | 41.110 | 21.274 | 10.468 | 1.00 | 33.51 |
| ATOM | 4562 | N | ASN | 2193 | 39.061 | 22.128 | 10.709 | 1.00 | 34.95 |
| ATOM | 4563 | CA | ASN | 2193 | 39.539 | 23.418 | 11.177 | 1.00 | 36.22 |
| ATOM | 4564 | CB | ASN | 2193 | 40.297 | 23.280 | 12.501 | 1.00 | 36.40 |
| ATOM | 4565 | CG | ASN | 2193 | 39.446 | 22.701 | 13.613 | 1.00 | 37.32 |
| ATOM | 4566 | OD1 | ASN | 2193 | 38.274 | 23.063 | 13.779 | 1.00 | 38.01 |
| ATOM | 4567 | ND2 | ASN | 2193 | 40.037 | 21.805 | 14.398 | 1.00 | 36.47 |
| ATOM | 4568 | C | ASN | 2193 | 40.455 | 24.072 | 10.139 | 1.00 | 37.23 |
| ATOM | 4569 | O | ASN | 2193 | 41.493 | 24.644 | 10.479 | 1.00 | 37.29 |
| ATOM | 4570 | N | GLY | 2194 | 40.071 | 23.978 | 8.871 | 1.00 | 37.92 |
| ATOM | 4571 | CA | GLY | 2194 | 40.851 | 24.586 | 7.809 | 1.00 | 37.94 |
| ATOM | 4572 | C | GLY | 2194 | 42.102 | 23.860 | 7.356 | 1.00 | 38.70 |
| ATOM | 4573 | O | GLY | 2194 | 42.680 | 24.209 | 6.330 | 1.00 | 39.05 |
| ATOM | 4574 | N | LYS | 2195 | 42.542 | 22.859 | 8.105 | 1.00 | 38.75 |
| ATOM | 4575 | CA | LYS | 2195 | 43.742 | 22.130 | 7.716 | 1.00 | 39.17 |
| ATOM | 4576 | CB | LYS | 2195 | 44.643 | 21.901 | 8.937 | 1.00 | 37.81 |
| ATOM | 4577 | C | LYS | 2195 | 43.407 | 20.798 | 7.051 | 1.00 | 39.81 |
| ATOM | 4578 | O | LYS | 2195 | 42.247 | 20.397 | 6.978 | 1.00 | 40.00 |
| ATOM | 4579 | N | GLU | 2196 | 44.433 | 20.118 | 6.555 | 1.00 | 40.79 |
| ATOM | 4580 | CA | GLU | 2196 | 44.243 | 18.830 | 5.907 | 1.00 | 41.19 |
| ATOM | 4581 | CB | GLU | 2196 | 45.556 | 18.372 | 5.270 | 1.00 | 42.59 |
| ATOM | 4582 | CG | GLU | 2196 | 45.501 | 16.989 | 4.612 | 1.00 | 44.96 |
| ATOM | 4583 | CD | GLU | 2196 | 46.795 | 16.630 | 3.881 | 1.00 | 46.10 |
| ATOM | 4584 | OE1 | GLU | 2196 | 47.873 | 16.638 | 4.523 | 1.00 | 46.26 |
| ATOM | 4585 | OE2 | GLU | 2196 | 46.728 | 16.339 | 2.664 | 1.00 | 46.71 |
| ATOM | 4586 | C | GLU | 2196 | 43.796 | 17.830 | 6.960 | 1.00 | 40.77 |
| ATOM | 4587 | O | GLU | 2196 | 44.272 | 17.865 | 8.096 | 1.00 | 40.55 |
| ATOM | 4588 | N | PHE | 2197 | 42.881 | 16.943 | 6.585 | 1.00 | 40.31 |
| ATOM | 4589 | CA | PHE | 2197 | 42.374 | 15.939 | 7.509 | 1.00 | 39.98 |
| ATOM | 4590 | CB | PHE | 2197 | 40.849 | 16.080 | 7.654 | 1.00 | 38.88 |
| ATOM | 4591 | CG | PHE | 2197 | 40.260 | 15.284 | 8.794 | 1.00 | 37.45 |
| ATOM | 4592 | CD1 | PHE | 2197 | 38.970 | 14.767 | 8.701 | 1.00 | 36.52 |
| ATOM | 4593 | CD2 | PHE | 2197 | 40.981 | 15.074 | 9.968 | 1.00 | 37.10 |
| ATOM | 4594 | CE1 | PHE | 2197 | 38.401 | 14.054 | 9.753 | 1.00 | 35.13 |
| ATOM | 4595 | CE2 | PHE | 2197 | 40.421 | 14.359 | 11.032 | 1.00 | 36.46 |
| ATOM | 4596 | CZ | PHE | 2197 | 39.127 | 13.848 | 10.921 | 1.00 | 35.64 |
| ATOM | 4597 | C | PHE | 2197 | 42.720 | 14.540 | 7.003 | 1.00 | 40.43 |
| ATOM | 4598 | O | PHE | 2197 | 42.274 | 14.133 | 5.931 | 1.00 | 40.64 |
| ATOM | 4599 | N | LYS | 2198 | 43.522 | 13.813 | 7.775 | 1.00 | 40.97 |
| ATOM | 4600 | CA | LYS | 2198 | 43.928 | 12.457 | 7.422 | 1.00 | 41.19 |
| ATOM | 4601 | CB | LYS | 2198 | 45.453 | 12.307 | 7.483 | 1.00 | 42.21 |
| ATOM | 4602 | CG | LYS | 2198 | 46.248 | 13.122 | 6.473 | 1.00 | 45.12 |
| ATOM | 4603 | CD | LYS | 2198 | 47.749 | 12.797 | 6.563 | 1.00 | 46.78 |
| ATOM | 4604 | CE | LYS | 2198 | 48.583 | 13.659 | 5.612 | 1.00 | 48.13 |
| ATOM | 4605 | NZ | LYS | 2198 | 50.057 | 13.393 | 5.734 | 1.00 | 49.26 |
| ATOM | 4606 | C | LYS | 2198 | 43.328 | 11.457 | 8.399 | 1.00 | 40.67 |
| ATOM | 4607 | O | LYS | 2198 | 43.251 | 11.713 | 9.593 | 1.00 | 40.71 |
| ATOM | 4608 | N | PRO | 2199 | 42.909 | 10.294 | 7.901 | 1.00 | 40.52 |
| ATOM | 4609 | CD | PRO | 2199 | 42.843 | 9.905 | 6.480 | 1.00 | 40.17 |
| ATOM | 4610 | CA | PRO | 2199 | 42.328 | 9.254 | 8.752 | 1.00 | 40.50 |
| ATOM | 4611 | CB | PRO | 2199 | 42.288 | 8.051 | 7.820 | 1.00 | 40.17 |
| ATOM | 4612 | CG | PRO | 2199 | 41.941 | 8.684 | 6.510 | 1.00 | 39.88 |
| ATOM | 4613 | C | PRO | 2199 | 43.140 | 8.979 | 10.013 | 1.00 | 40.66 |
| ATOM | 4614 | O | PRO | 2199 | 42.578 | 8.663 | 11.060 | 1.00 | 40.75 |
| ATOM | 4615 | N | ASP | 2200 | 44.462 | 9.099 | 9.923 | 1.00 | 41.02 |
| ATOM | 4616 | CA | ASP | 2200 | 45.299 | 8.842 | 11.086 | 1.00 | 41.36 |
| ATOM | 4617 | CB | ASP | 2200 | 46.760 | 8.670 | 10.687 | 1.00 | 43.35 |
| ATOM | 4618 | CG | ASP | 2200 | 47.052 | 7.288 | 10.140 | 1.00 | 46.00 |
| ATOM | 4619 | OD1 | ASP | 2200 | 46.331 | 6.335 | 10.510 | 1.00 | 47.14 |
| ATOM | 4620 | OD2 | ASP | 2200 | 48.018 | 7.146 | 9.354 | 1.00 | 48.08 |
| ATOM | 4621 | C | ASP | 2200 | 45.202 | 9.925 | 12.139 | 1.00 | 40.96 |
| ATOM | 4622 | O | ASP | 2200 | 45.840 | 9.824 | 13.188 | 1.00 | 40.84 |
| ATOM | 4623 | N | HIS | 2201 | 44.408 | 10.958 | 11.876 | 1.00 | 40.03 |
| ATOM | 4624 | CA | HIS | 2201 | 44.281 | 12.026 | 12.852 | 1.00 | 39.15 |
| ATOM | 4625 | CB | HIS | 2201 | 43.845 | 13.323 | 12.183 | 1.00 | 39.13 |
| ATOM | 4626 | CG | HIS | 2201 | 44.881 | 13.897 | 11.274 | 1.00 | 38.72 |
| ATOM | 4627 | CD2 | HIS | 2201 | 44.798 | 14.839 | 10.305 | 1.00 | 39.08 |
| ATOM | 4628 | ND1 | HIS | 2201 | 46.201 | 13.512 | 11.323 | 1.00 | 38.66 |
| ATOM | 4629 | CE1 | HIS | 2201 | 46.889 | 14.192 | 10.424 | 1.00 | 38.95 |
| ATOM | 4630 | NE2 | HIS | 2201 | 46.061 | 15.004 | 9.793 | 1.00 | 38.90 |
| ATOM | 4631 | C | HIS | 2201 | 43.345 | 11.663 | 13.991 | 1.00 | 38.44 |
| ATOM | 4632 | O | HIS | 2201 | 43.240 | 12.393 | 14.974 | 1.00 | 38.68 |
| ATOM | 4633 | N | ARG | 2202 | 42.672 | 10.529 | 13.872 | 1.00 | 37.17 |
| ATOM | 4634 | CA | ARG | 2202 | 41.782 | 10.101 | 14.936 | 1.00 | 36.72 |
| ATOM | 4635 | CB | ARG | 2202 | 40.361 | 10.583 | 14.671 | 1.00 | 35.70 |
| ATOM | 4636 | CG | ARG | 2202 | 39.625 | 9.829 | 13.601 | 1.00 | 34.24 |
| ATOM | 4637 | CD | ARG | 2202 | 38.337 | 10.547 | 13.308 | 1.00 | 35.17 |
| ATOM | 4638 | NE | ARG | 2202 | 37.538 | 10.757 | 14.512 | 1.00 | 34.44 |
| ATOM | 4639 | CZ | ARG | 2202 | 36.628 | 9.902 | 14.965 | 1.00 | 33.51 |
| ATOM | 4640 | NH1 | ARG | 2202 | 36.397 | 8.775 | 14.314 | 1.00 | 33.29 |
| ATOM | 4641 | NH2 | ARG | 2202 | 35.938 | 10.180 | 16.061 | 1.00 | 33.23 |
| ATOM | 4642 | C | ARG | 2202 | 41.805 | 8.587 | 15.036 | 1.00 | 37.41 |
| ATOM | 4643 | O | ARG | 2202 | 41.929 | 7.898 | 14.023 | 1.00 | 37.43 |
| ATOM | 4644 | N | ILE | 2203 | 41.701 | 8.057 | 16.250 | 1.00 | 37.98 |
| ATOM | 4645 | CA | ILE | 2203 | 41.720 | 6.613 | 16.382 | 1.00 | 39.08 |
| ATOM | 4646 | CB | ILE | 2203 | 41.743 | 6.156 | 17.859 | 1.00 | 39.38 |
| ATOM | 4647 | CG2 | ILE | 2203 | 40.365 | 6.320 | 18.491 | 1.00 | 39.25 |
| ATOM | 4648 | CG1 | ILE | 2203 | 42.163 | 4.682 | 17.927 | 1.00 | 40.24 |
| ATOM | 4649 | CD1 | ILE | 2203 | 43.505 | 4.368 | 17.263 | 1.00 | 39.74 |
| ATOM | 4650 | C | ILE | 2203 | 40.505 | 6.032 | 15.655 | 1.00 | 39.93 |
| ATOM | 4651 | O | ILE | 2203 | 39.357 | 6.480 | 15.836 | 1.00 | 39.85 |
| ATOM | 4652 | N | GLY | 2204 | 40.778 | 5.045 | 14.806 | 1.00 | 40.11 |
| ATOM | 4653 | CA | GLY | 2204 | 39.722 | 4.421 | 14.033 | 1.00 | 40.06 |
| ATOM | 4654 | C | GLY | 2204 | 39.524 | 5.131 | 12.705 | 1.00 | 40.07 |
| ATOM | 4655 | O | GLY | 2204 | 38.818 | 4.633 | 11.832 | 1.00 | 40.29 |
| ATOM | 4656 | N | GLY | 2205 | 40.146 | 6.298 | 12.557 | 1.00 | 39.47 |
| ATOM | 4657 | CA | GLY | 2205 | 40.028 | 7.063 | 11.331 | 1.00 | 38.74 |
| ATOM | 4658 | C | GLY | 2205 | 38.599 | 7.431 | 10.974 | 1.00 | 39.22 |
| ATOM | 4659 | O | GLY | 2205 | 37.731 | 7.551 | 11.838 | 1.00 | 38.55 |
| ATOM | 4660 | N | TYR | 2206 | 38.361 | 7.619 | 9.680 | 1.00 | 39.35 |
| ATOM | 4661 | CA | TYR | 2206 | 37.039 | 7.965 | 9.184 | 1.00 | 39.83 |
| ATOM | 4662 | CB | TYR | 2206 | 36.872 | 9.478 | 9.125 | 1.00 | 39.10 |
| ATOM | 4663 | CG | TYR | 2206 | 37.891 | 10.192 | 8.270 | 1.00 | 38.21 |
| ATOM | 4664 | CD1 | TYR | 2206 | 39.018 | 10.792 | 8.841 | 1.00 | 38.18 |
| ATOM | 4665 | CE1 | TYR | 2206 | 39.927 | 11.514 | 8.054 | 1.00 | 38.28 |
| ATOM | 4666 | CD2 | TYR | 2206 | 37.701 | 10.317 | 6.894 | 1.00 | 38.11 |
| ATOM | 4667 | CE2 | TYR | 2206 | 38.594 | 11.026 | 6.103 | 1.00 | 37.47 |
| ATOM | 4668 | CZ | TYR | 2206 | 39.702 | 11.627 | 6.683 | 1.00 | 38.27 |
| ATOM | 4669 | OH | TYR | 2206 | 40.563 | 12.356 | 5.891 | 1.00 | 38.52 |
| ATOM | 4670 | C | TYR | 2206 | 36.856 | 7.386 | 7.791 | 1.00 | 40.92 |
| ATOM | 4671 | O | TYR | 2206 | 37.812 | 6.910 | 7.184 | 1.00 | 41.39 |
| ATOM | 4672 | N | LYS | 2207 | 35.633 | 7.416 | 7.275 | 1.00 | 41.44 |
| ATOM | 4673 | CA | LYS | 2207 | 35.418 | 6.892 | 5.943 | 1.00 | 42.13 |
| ATOM | 4674 | CB | LYS | 2207 | 34.636 | 5.574 | 5.980 | 1.00 | 43.23 |
| ATOM | 4675 | CG | LYS | 2207 | 34.664 | 4.803 | 4.648 | 1.00 | 45.01 |
| ATOM | 4676 | CD | LYS | 2207 | 34.572 | 3.284 | 4.880 | 1.00 | 47.43 |
| ATOM | 4677 | CE | LYS | 2207 | 34.652 | 2.460 | 3.584 | 1.00 | 48.14 |
| ATOM | 4678 | NZ | LYS | 2207 | 34.582 | 0.977 | 3.861 | 1.00 | 48.95 |
| ATOM | 4679 | C | LYS | 2207 | 34.721 | 7.898 | 5.051 | 1.00 | 42.14 |
| ATOM | 4680 | O | LYS | 2207 | 33.757 | 8.553 | 5.457 | 1.00 | 42.22 |
| ATOM | 4681 | N | VAL | 2208 | 35.240 | 8.032 | 3.835 | 1.00 | 41.86 |
| ATOM | 4682 | CA | VAL | 2208 | 34.670 | 8.945 | 2.853 | 1.00 | 41.71 |
| ATOM | 4683 | CB | VAL | 2208 | 35.733 | 9.872 | 2.216 | 1.00 | 41.10 |
| ATOM | 4684 | CG1 | VAL | 2208 | 35.090 | 10.713 | 1.122 | 1.00 | 39.60 |
| ATOM | 4685 | CG2 | VAL | 2208 | 36.356 | 10.773 | 3.275 | 1.00 | 40.32 |
| ATOM | 4686 | C | VAL | 2208 | 34.041 | 8.135 | 1.738 | 1.00 | 42.20 |
| ATOM | 4687 | O | VAL | 2208 | 34.737 | 7.458 | 0.993 | 1.00 | 42.69 |
| ATOM | 4688 | N | ARG | 2209 | 32.721 | 8.189 | 1.643 | 1.00 | 42.87 |
| ATOM | 4689 | CA | ARG | 2209 | 31.991 | 7.491 | 0.596 | 1.00 | 43.67 |
| ATOM | 4690 | CB | ARG | 2209 | 30.651 | 6.975 | 1.129 | 1.00 | 45.79 |
| ATOM | 4691 | CG | ARG | 2209 | 30.475 | 5.463 | 1.113 | 1.00 | 49.62 |
| ATOM | 4692 | CD | ARG | 2209 | 31.078 | 4.813 | −0.152 | 1.00 | 53.25 |
| ATOM | 4693 | NE | ARG | 2209 | 32.438 | 4.305 | 0.080 | 1.00 | 55.06 |
| ATOM | 4694 | CZ | ARG | 2209 | 33.445 | 4.410 | −0.784 | 1.00 | 55.10 |
| ATOM | 4695 | NH1 | ARG | 2209 | 33.263 | 5.008 | −1.954 | 1.00 | 54.30 |
| ATOM | 4696 | NH2 | ARG | 2209 | 34.638 | 3.922 | −0.469 | 1.00 | 55.63 |
| ATOM | 4697 | C | ARG | 2209 | 31.730 | 8.546 | −0.469 | 1.00 | 43.06 |
| ATOM | 4698 | O | ARG | 2209 | 30.834 | 9.375 | −0.318 | 1.00 | 43.07 |
| ATOM | 4699 | N | TYR | 2210 | 32.508 | 8.517 | −1.543 | 1.00 | 42.43 |
| ATOM | 4700 | CA | TYR | 2210 | 32.365 | 9.503 | −2.603 | 1.00 | 41.19 |
| ATOM | 4701 | CB | TYR | 2210 | 33.528 | 9.353 | −3.584 | 1.00 | 39.09 |
| ATOM | 4702 | CG | TYR | 2210 | 34.859 | 9.509 | −2.881 | 1.00 | 37.99 |
| ATOM | 4703 | CD1 | TYR | 2210 | 35.490 | 8.417 | −2.280 | 1.00 | 37.46 |
| ATOM | 4704 | CE1 | TYR | 2210 | 36.665 | 8.581 | −1.555 | 1.00 | 37.15 |
| ATOM | 4705 | CD2 | TYR | 2210 | 35.445 | 10.766 | −2.741 | 1.00 | 37.16 |
| ATOM | 4706 | CE2 | TYR | 2210 | 36.607 | 10.938 | −2.023 | 1.00 | 36.76 |
| ATOM | 4707 | CZ | TYR | 2210 | 37.214 | 9.851 | −1.428 | 1.00 | 37.26 |
| ATOM | 4708 | OH | TYR | 2210 | 38.358 | 10.054 | −0.684 | 1.00 | 37.31 |
| ATOM | 4709 | C | TYR | 2210 | 31.030 | 9.457 | −3.317 | 1.00 | 40.83 |
| ATOM | 4710 | O | TYR | 2210 | 30.536 | 10.493 | −3.775 | 1.00 | 41.54 |
| ATOM | 4711 | N | ALA | 2211 | 30.437 | 8.270 | −3.397 | 1.00 | 40.03 |
| ATOM | 4712 | CA | ALA | 2211 | 29.146 | 8.127 | −4.067 | 1.00 | 39.77 |
| ATOM | 4713 | CB | ALA | 2211 | 28.725 | 6.658 | −4.084 | 1.00 | 39.04 |
| ATOM | 4714 | C | ALA | 2211 | 28.072 | 8.975 | −3.378 | 1.00 | 39.57 |
| ATOM | 4715 | O | ALA | 2211 | 27.156 | 9.489 | −4.028 | 1.00 | 39.59 |
| ATOM | 4716 | N | THR | 2212 | 28.198 | 9.127 | −2.064 | 1.00 | 38.75 |
| ATOM | 4717 | CA | THR | 2212 | 27.235 | 9.900 | −1.295 | 1.00 | 38.30 |
| ATOM | 4718 | CB | THR | 2212 | 26.733 | 9.098 | −0.060 | 1.00 | 38.57 |
| ATOM | 4719 | OG1 | THR | 2212 | 27.854 | 8.649 | 0.714 | 1.00 | 39.63 |
| ATOM | 4720 | CG2 | THR | 2212 | 25.924 | 7.887 | −0.495 | 1.00 | 37.48 |
| ATOM | 4721 | C | THR | 2212 | 27.799 | 11.230 | −0.809 | 1.00 | 38.18 |
| ATOM | 4722 | O | THR | 2212 | 27.165 | 11.919 | −0.012 | 1.00 | 38.33 |
| ATOM | 4723 | N | TRP | 2213 | 28.989 | 11.587 | −1.287 | 1.00 | 37.72 |
| ATOM | 4724 | CA | TRP | 2213 | 29.634 | 12.838 | −0.898 | 1.00 | 37.00 |
| ATOM | 4725 | CB | TRP | 2213 | 28.923 | 14.025 | −1.545 | 1.00 | 36.92 |
| ATOM | 4726 | CG | TRP | 2213 | 28.820 | 13.879 | −3.012 | 1.00 | 37.85 |
| ATOM | 4727 | CD2 | TRP | 2213 | 29.714 | 14.418 | −3.988 | 1.00 | 37.80 |
| ATOM | 4728 | CE2 | TRP | 2213 | 29.277 | 13.964 | −5.254 | 1.00 | 38.22 |
| ATOM | 4729 | CE3 | TRP | 2213 | 30.849 | 15.240 | −3.920 | 1.00 | 38.14 |
| ATOM | 4730 | CD1 | TRP | 2213 | 27.897 | 13.141 | −3.703 | 1.00 | 38.23 |
| ATOM | 4731 | NE1 | TRP | 2213 | 28.165 | 13.185 | −5.049 | 1.00 | 37.28 |
| ATOM | 4732 | CZ2 | TRP | 2213 | 29.934 | 14.305 | −6.445 | 1.00 | 37.74 |
| ATOM | 4733 | CZ3 | TRP | 2213 | 31.505 | 15.580 | −5.106 | 1.00 | 37.85 |
| ATOM | 4734 | CH2 | TRP | 2213 | 31.043 | 15.111 | −6.350 | 1.00 | 37.54 |
| ATOM | 4735 | C | TRP | 2213 | 29.589 | 12.985 | 0.610 | 1.00 | 36.70 |
| ATOM | 4736 | O | TRP | 2213 | 29.217 | 14.034 | 1.130 | 1.00 | 36.53 |
| ATOM | 4737 | N | SER | 2214 | 29.987 | 11.932 | 1.310 | 1.00 | 36.52 |
| ATOM | 4738 | CA | SER | 2214 | 29.944 | 11.934 | 2.770 | 1.00 | 36.47 |
| ATOM | 4739 | CB | SER | 2214 | 28.869 | 10.972 | 3.278 | 1.00 | 36.34 |
| ATOM | 4740 | OG | SER | 2214 | 27.706 | 11.032 | 2.485 | 1.00 | 39.55 |
| ATOM | 4741 | C | SER | 2214 | 31.222 | 11.504 | 3.446 | 1.00 | 35.38 |
| ATOM | 4742 | O | SER | 2214 | 32.053 | 10.814 | 2.864 | 1.00 | 35.14 |
| ATOM | 4743 | N | ILE | 2215 | 31.340 | 11.907 | 4.704 | 1.00 | 34.01 |
| ATOM | 4744 | CA | ILE | 2215 | 32.459 | 11.531 | 5.538 | 1.00 | 33.45 |
| ATOM | 4745 | CB | ILE | 2215 | 33.389 | 12.736 | 5.863 | 1.00 | 33.89 |
| ATOM | 4746 | CG2 | ILE | 2215 | 32.612 | 13.861 | 6.543 | 1.00 | 33.90 |
| ATOM | 4747 | CG1 | ILE | 2215 | 34.533 | 12.268 | 6.763 | 1.00 | 33.44 |
| ATOM | 4748 | CD1 | ILE | 2215 | 35.590 | 13.321 | 7.025 | 1.00 | 33.51 |
| ATOM | 4749 | C | ILE | 2215 | 31.786 | 11.007 | 6.802 | 1.00 | 33.08 |
| ATOM | 4750 | O | ILE | 2215 | 30.842 | 11.620 | 7.305 | 1.00 | 33.16 |
| ATOM | 4751 | N | ILE | 2216 | 32.243 | 9.859 | 7.289 | 1.00 | 32.46 |
| ATOM | 4752 | CA | ILE | 2216 | 31.659 | 9.258 | 8.478 | 1.00 | 32.10 |
| ATOM | 4753 | CB | ILE | 2216 | 31.085 | 7.858 | 8.201 | 1.00 | 32.72 |
| ATOM | 4754 | CG2 | ILE | 2216 | 30.077 | 7.506 | 9.272 | 1.00 | 32.36 |
| ATOM | 4755 | CG1 | ILE | 2216 | 30.446 | 7.798 | 6.813 | 1.00 | 32.60 |
| ATOM | 4756 | CD1 | ILE | 2216 | 29.437 | 8.855 | 6.573 | 1.00 | 32.91 |
| ATOM | 4757 | C | ILE | 2216 | 32.692 | 9.082 | 9.569 | 1.00 | 32.33 |
| ATOM | 4758 | O | ILE | 2216 | 33.785 | 8.571 | 9.334 | 1.00 | 32.56 |
| ATOM | 4759 | N | MSE | 2217 | 32.349 | 9.517 | 10.768 | 1.00 | 32.03 |
| ATOM | 4760 | CA | MSE | 2217 | 33.250 | 9.362 | 11.893 | 1.00 | 32.22 |
| ATOM | 4761 | CB | MSE | 2217 | 33.632 | 10.721 | 12.501 | 1.00 | 32.33 |
| ATOM | 4762 | CG | MSE | 2217 | 34.537 | 11.597 | 11.636 | 1.00 | 32.35 |
| ATOM | 4763 | SE | MSE | 2217 | 35.099 | 13.110 | 12.484 | 1.00 | 31.09 |
| ATOM | 4764 | CE | MSE | 2217 | 33.966 | 14.320 | 11.777 | 1.00 | 32.29 |
| ATOM | 4765 | C | MSE | 2217 | 32.504 | 8.527 | 12.925 | 1.00 | 32.43 |
| ATOM | 4766 | O | MSE | 2217 | 31.415 | 8.913 | 13.373 | 1.00 | 32.63 |
| ATOM | 4767 | N | ASP | 2218 | 33.060 | 7.367 | 13.276 | 1.00 | 31.94 |
| ATOM | 4768 | CA | ASP | 2218 | 32.425 | 6.523 | 14.284 | 1.00 | 31.62 |
| ATOM | 4769 | CB | ASP | 2218 | 32.741 | 5.034 | 14.096 | 1.00 | 32.01 |
| ATOM | 4770 | CG | ASP | 2218 | 31.762 | 4.337 | 13.163 | 1.00 | 34.43 |
| ATOM | 4771 | OD1 | ASP | 2218 | 30.640 | 4.862 | 12.982 | 1.00 | 36.54 |
| ATOM | 4772 | OD2 | ASP | 2218 | 32.098 | 3.259 | 12.618 | 1.00 | 34.27 |
| ATOM | 4773 | C | ASP | 2218 | 32.897 | 6.938 | 15.664 | 1.00 | 30.95 |
| ATOM | 4774 | O | ASP | 2218 | 34.019 | 7.396 | 15.834 | 1.00 | 30.16 |
| ATOM | 4775 | N | SER | 2219 | 32.011 | 6.794 | 16.643 | 1.00 | 31.38 |
| ATOM | 4776 | CA | SER | 2219 | 32.302 | 7.108 | 18.040 | 1.00 | 30.72 |
| ATOM | 4777 | CB | SER | 2219 | 33.119 | 5.961 | 18.642 | 1.00 | 30.89 |
| ATOM | 4778 | OG | SER | 2219 | 33.430 | 6.206 | 20.005 | 1.00 | 33.89 |
| ATOM | 4779 | C | SER | 2219 | 33.032 | 8.435 | 18.284 | 1.00 | 30.40 |
| ATOM | 4780 | O | SER | 2219 | 34.166 | 8.451 | 18.765 | 1.00 | 30.11 |
| ATOM | 4781 | N | VAL | 2220 | 32.387 | 9.556 | 17.980 | 1.00 | 29.82 |
| ATOM | 4782 | CA | VAL | 2220 | 33.046 | 10.842 | 18.186 | 1.00 | 29.20 |
| ATOM | 4783 | CB | VAL | 2220 | 32.201 | 12.023 | 17.666 | 1.00 | 28.49 |
| ATOM | 4784 | CG1 | VAL | 2220 | 32.008 | 11.890 | 16.171 | 1.00 | 29.39 |
| ATOM | 4785 | CG2 | VAL | 2220 | 30.858 | 12.066 | 18.375 | 1.00 | 27.85 |
| ATOM | 4786 | C | VAL | 2220 | 33.363 | 11.108 | 19.653 | 1.00 | 29.37 |
| ATOM | 4787 | O | VAL | 2220 | 32.656 | 10.648 | 20.553 | 1.00 | 29.99 |
| ATOM | 4788 | N | VAL | 2221 | 34.445 | 11.838 | 19.895 | 1.00 | 28.16 |
| ATOM | 4789 | CA | VAL | 2221 | 34.815 | 12.190 | 21.254 | 1.00 | 27.89 |
| ATOM | 4790 | CB | VAL | 2221 | 35.972 | 11.320 | 21.816 | 1.00 | 27.85 |
| ATOM | 4791 | CG1 | VAL | 2221 | 35.480 | 9.897 | 21.995 | 1.00 | 28.04 |
| ATOM | 4792 | CG2 | VAL | 2221 | 37.193 | 11.363 | 20.891 | 1.00 | 27.75 |
| ATOM | 4793 | C | VAL | 2221 | 35.185 | 13.660 | 21.257 | 1.00 | 27.74 |
| ATOM | 4794 | O | VAL | 2221 | 35.314 | 14.274 | 20.200 | 1.00 | 27.91 |
| ATOM | 4795 | N | PRO | 2222 | 35.331 | 14.257 | 22.446 | 1.00 | 27.31 |
| ATOM | 4796 | CD | PRO | 2222 | 35.131 | 13.669 | 23.780 | 1.00 | 28.60 |
| ATOM | 4797 | CA | PRO | 2222 | 35.679 | 15.675 | 22.566 | 1.00 | 27.86 |
| ATOM | 4798 | CB | PRO | 2222 | 36.104 | 15.802 | 24.021 | 1.00 | 26.70 |
| ATOM | 4799 | CG | PRO | 2222 | 35.156 | 14.897 | 24.697 | 1.00 | 27.46 |
| ATOM | 4800 | C | PRO | 2222 | 36.740 | 16.211 | 21.600 | 1.00 | 28.28 |
| ATOM | 4801 | O | PRO | 2222 | 36.608 | 17.324 | 21.084 | 1.00 | 27.38 |
| ATOM | 4802 | N | SER | 2223 | 37.786 | 15.428 | 21.348 | 1.00 | 28.73 |
| ATOM | 4803 | CA | SER | 2223 | 38.839 | 15.881 | 20.450 | 1.00 | 29.47 |
| ATOM | 4804 | CB | SER | 2223 | 40.075 | 14.993 | 20.584 | 1.00 | 30.01 |
| ATOM | 4805 | OG | SER | 2223 | 39.780 | 13.649 | 20.255 | 1.00 | 31.64 |
| ATOM | 4806 | C | SER | 2223 | 38.398 | 15.941 | 18.987 | 1.00 | 30.05 |
| ATOM | 4807 | O | SER | 2223 | 39.156 | 16.385 | 18.129 | 1.00 | 30.31 |
| ATOM | 4808 | N | ASP | 2224 | 37.182 | 15.496 | 18.691 | 1.00 | 30.07 |
| ATOM | 4809 | CA | ASP | 2224 | 36.706 | 15.570 | 17.319 | 1.00 | 30.52 |
| ATOM | 4810 | CB | ASP | 2224 | 35.785 | 14.389 | 16.990 | 1.00 | 31.19 |
| ATOM | 4811 | CG | ASP | 2224 | 36.525 | 13.049 | 17.007 | 1.00 | 31.26 |
| ATOM | 4812 | OD1 | ASP | 2224 | 37.579 | 12.946 | 16.350 | 1.00 | 30.69 |
| ATOM | 4813 | OD2 | ASP | 2224 | 36.058 | 12.103 | 17.677 | 1.00 | 31.47 |
| ATOM | 4814 | C | ASP | 2224 | 35.974 | 16.888 | 17.116 | 1.00 | 30.77 |
| ATOM | 4815 | O | ASP | 2224 | 35.614 | 17.234 | 15.993 | 1.00 | 30.77 |
| ATOM | 4816 | N | LYS | 2225 | 35.763 | 17.629 | 18.204 | 1.00 | 31.05 |
| ATOM | 4817 | CA | LYS | 2225 | 35.078 | 18.913 | 18.112 | 1.00 | 31.24 |
| ATOM | 4818 | CB | LYS | 2225 | 34.977 | 19.587 | 19.482 | 1.00 | 31.76 |
| ATOM | 4819 | CG | LYS | 2225 | 34.106 | 18.836 | 20.472 | 1.00 | 33.15 |
| ATOM | 4820 | CD | LYS | 2225 | 33.373 | 19.766 | 21.423 | 1.00 | 33.22 |
| ATOM | 4821 | CE | LYS | 2225 | 34.309 | 20.421 | 22.416 | 1.00 | 34.64 |
| ATOM | 4822 | NZ | LYS | 2225 | 33.538 | 21.135 | 23.477 | 1.00 | 34.82 |
| ATOM | 4823 | C | LYS | 2225 | 35.809 | 19.838 | 17.144 | 1.00 | 31.39 |
| ATOM | 4824 | O | LYS | 2225 | 37.038 | 19.884 | 17.133 | 1.00 | 31.58 |
| ATOM | 4825 | N | GLY | 2226 | 35.052 | 20.561 | 16.325 | 1.00 | 30.81 |
| ATOM | 4826 | CA | GLY | 2226 | 35.671 | 21.460 | 15.378 | 1.00 | 31.22 |
| ATOM | 4827 | C | GLY | 2226 | 34.829 | 21.724 | 14.153 | 1.00 | 31.51 |
| ATOM | 4828 | O | GLY | 2226 | 33.686 | 21.287 | 14.065 | 1.00 | 31.85 |
| ATOM | 4829 | N | ASN | 2227 | 35.408 | 22.449 | 13.205 | 1.00 | 31.79 |
| ATOM | 4830 | CA | ASN | 2227 | 34.729 | 22.786 | 11.970 | 1.00 | 32.38 |
| ATOM | 4831 | CB | ASN | 2227 | 35.043 | 24.236 | 11.579 | 1.00 | 32.35 |
| ATOM | 4832 | CG | ASN | 2227 | 34.314 | 25.243 | 12.452 | 1.00 | 34.00 |
| ATOM | 4833 | OD1 | ASN | 2227 | 33.081 | 25.212 | 12.559 | 1.00 | 34.93 |
| ATOM | 4834 | ND2 | ASN | 2227 | 35.067 | 26.143 | 13.082 | 1.00 | 34.13 |
| ATOM | 4835 | C | ASN | 2227 | 35.187 | 21.846 | 10.872 | 1.00 | 32.37 |
| ATOM | 4836 | O | ASN | 2227 | 36.382 | 21.626 | 10.694 | 1.00 | 32.62 |
| ATOM | 4837 | N | TYR | 2228 | 34.242 | 21.278 | 10.138 | 1.00 | 31.82 |
| ATOM | 4838 | CA | TYR | 2228 | 34.614 | 20.389 | 9.058 | 1.00 | 31.42 |
| ATOM | 4839 | CB | TYR | 2228 | 34.048 | 18.995 | 9.289 | 1.00 | 29.67 |
| ATOM | 4840 | CG | TYR | 2228 | 34.669 | 18.302 | 10.475 | 1.00 | 28.19 |
| ATOM | 4841 | CD1 | TYR | 2228 | 34.271 | 18.610 | 11.777 | 1.00 | 27.01 |
| ATOM | 4842 | CE1 | TYR | 2228 | 34.842 | 17.957 | 12.874 | 1.00 | 27.51 |
| ATOM | 4843 | CD2 | TYR | 2228 | 35.657 | 17.327 | 10.293 | 1.00 | 27.73 |
| ATOM | 4844 | CE2 | TYR | 2228 | 36.236 | 16.665 | 11.377 | 1.00 | 27.75 |
| ATOM | 4845 | CZ | TYR | 2228 | 35.826 | 16.980 | 12.666 | 1.00 | 27.54 |
| ATOM | 4846 | OH | TYR | 2228 | 36.395 | 16.316 | 13.732 | 1.00 | 27.00 |
| ATOM | 4847 | C | TYR | 2228 | 34.122 | 20.982 | 7.760 | 1.00 | 32.24 |
| ATOM | 4848 | O | TYR | 2228 | 32.936 | 21.262 | 7.590 | 1.00 | 31.64 |
| ATOM | 4849 | N | THR | 2229 | 35.062 | 21.192 | 6.847 | 1.00 | 33.42 |
| ATOM | 4850 | CA | THR | 2229 | 34.754 | 21.790 | 5.563 | 1.00 | 34.80 |
| ATOM | 4851 | CB | THR | 2229 | 35.627 | 23.015 | 5.307 | 1.00 | 35.15 |
| ATOM | 4852 | OG1 | THR | 2229 | 35.782 | 23.761 | 6.522 | 1.00 | 34.94 |
| ATOM | 4853 | CG2 | THR | 2229 | 34.983 | 23.895 | 4.237 | 1.00 | 35.02 |
| ATOM | 4854 | C | THR | 2229 | 34.981 | 20.827 | 4.416 | 1.00 | 35.94 |
| ATOM | 4855 | O | THR | 2229 | 36.013 | 20.163 | 4.338 | 1.00 | 36.48 |
| ATOM | 4856 | N | CYS | 2230 | 34.010 | 20.764 | 3.519 | 1.00 | 37.07 |
| ATOM | 4857 | CA | CYS | 2230 | 34.098 | 19.901 | 2.354 | 1.00 | 38.16 |
| ATOM | 4858 | CB | CYS | 2230 | 32.742 | 19.285 | 2.056 | 1.00 | 38.34 |
| ATOM | 4859 | SG | CYS | 2230 | 31.533 | 20.583 | 1.637 | 1.00 | 40.57 |
| ATOM | 4860 | C | CYS | 2230 | 34.452 | 20.827 | 1.212 | 1.00 | 38.69 |
| ATOM | 4861 | O | CYS | 2230 | 33.921 | 21.932 | 1.128 | 1.00 | 38.52 |
| ATOM | 4862 | N | ILE | 2231 | 35.336 | 20.368 | 0.337 | 1.00 | 39.59 |
| ATOM | 4863 | CA | ILE | 2231 | 35.751 | 21.147 | −0.812 | 1.00 | 40.84 |
| ATOM | 4864 | CB | ILE | 2231 | 37.237 | 21.539 | −0.668 | 1.00 | 40.86 |
| ATOM | 4865 | CG2 | ILE | 2231 | 37.772 | 22.116 | −1.976 | 1.00 | 41.15 |
| ATOM | 4866 | CG1 | ILE | 2231 | 37.369 | 22.555 | 0.472 | 1.00 | 40.07 |
| ATOM | 4867 | CD1 | ILE | 2231 | 38.780 | 22.781 | 0.939 | 1.00 | 40.47 |
| ATOM | 4868 | C | ILE | 2231 | 35.500 | 20.346 | −2.087 | 1.00 | 41.77 |
| ATOM | 4869 | O | ILE | 2231 | 36.165 | 19.351 | −2.353 | 1.00 | 41.85 |
| ATOM | 4870 | N | VAL | 2232 | 34.507 | 20.783 | −2.852 | 1.00 | 43.29 |
| ATOM | 4871 | CA | VAL | 2232 | 34.117 | 20.135 | −4.099 | 1.00 | 45.42 |
| ATOM | 4872 | CB | VAL | 2232 | 32.569 | 20.045 | −4.202 | 1.00 | 45.41 |
| ATOM | 4873 | CG1 | VAL | 2232 | 32.143 | 19.573 | −5.585 | 1.00 | 46.24 |
| ATOM | 4874 | CG2 | VAL | 2232 | 32.046 | 19.091 | −3.153 | 1.00 | 45.88 |
| ATOM | 4875 | C | VAL | 2232 | 34.646 | 20.960 | −5.265 | 1.00 | 46.67 |
| ATOM | 4876 | O | VAL | 2232 | 34.295 | 22.130 | −5.415 | 1.00 | 46.72 |
| ATOM | 4877 | N | GLU | 2233 | 35.478 | 20.359 | −6.101 | 1.00 | 48.22 |
| ATOM | 4878 | CA | GLU | 2233 | 36.023 | 21.109 | −7.217 | 1.00 | 50.18 |
| ATOM | 4879 | CB | GLU | 2233 | 37.237 | 21.914 | −6.735 | 1.00 | 51.32 |
| ATOM | 4880 | CG | GLU | 2233 | 38.386 | 21.060 | −6.194 | 1.00 | 53.80 |
| ATOM | 4881 | CD | GLU | 2233 | 39.312 | 21.834 | −5.247 | 1.00 | 55.20 |
| ATOM | 4882 | OE1 | GLU | 2233 | 39.546 | 23.037 | −5.500 | 1.00 | 55.90 |
| ATOM | 4883 | OE2 | GLU | 2233 | 39.811 | 21.236 | −4.258 | 1.00 | 55.21 |
| ATOM | 4884 | C | GLU | 2233 | 36.405 | 20.295 | −8.445 | 1.00 | 50.64 |
| ATOM | 4885 | O | GLU | 2233 | 36.634 | 19.085 | −8.375 | 1.00 | 49.97 |
| ATOM | 4886 | N | ASN | 2234 | 36.431 | 20.988 | −9.579 | 1.00 | 51.72 |
| ATOM | 4887 | CA | ASN | 2234 | 36.838 | 20.423 | −10.861 | 1.00 | 52.70 |
| ATOM | 4888 | CB | ASN | 2234 | 35.648 | 19.851 | −11.654 | 1.00 | 51.82 |
| ATOM | 4889 | CG | ASN | 2234 | 34.613 | 20.893 | −12.018 | 1.00 | 51.42 |
| ATOM | 4890 | OD1 | ASN | 2234 | 34.866 | 22.098 | −11.957 | 1.00 | 50.88 |
| ATOM | 4891 | ND2 | ASN | 2234 | 33.433 | 20.428 | −12.419 | 1.00 | 50.97 |
| ATOM | 4892 | C | ASN | 2234 | 37.510 | 21.572 | −11.617 | 1.00 | 53.87 |
| ATOM | 4893 | O | ASN | 2234 | 37.568 | 22.702 | −11.118 | 1.00 | 54.19 |
| ATOM | 4894 | N | GLU | 2235 | 38.025 | 21.293 | −12.806 | 1.00 | 54.95 |
| ATOM | 4895 | CA | GLU | 2235 | 38.706 | 22.316 | −13.598 | 1.00 | 56.07 |
| ATOM | 4896 | CB | GLU | 2235 | 38.988 | 21.779 | −14.999 | 1.00 | 57.22 |
| ATOM | 4897 | CG | GLU | 2235 | 39.889 | 20.564 | −15.039 | 1.00 | 59.36 |
| ATOM | 4898 | CD | GLU | 2235 | 39.716 | 19.761 | −16.329 | 1.00 | 61.03 |
| ATOM | 4899 | OE1 | GLU | 2235 | 38.649 | 19.117 | −16.496 | 1.00 | 61.00 |
| ATOM | 4900 | OE2 | GLU | 2235 | 40.642 | 19.784 | −17.176 | 1.00 | 61.61 |
| ATOM | 4901 | C | GLU | 2235 | 37.960 | 23.643 | −13.739 | 1.00 | 55.91 |
| ATOM | 4902 | O | GLU | 2235 | 38.584 | 24.694 | −13.884 | 1.00 | 56.22 |
| ATOM | 4903 | N | TYR | 2236 | 36.634 | 23.598 | −13.682 | 1.00 | 55.84 |
| ATOM | 4904 | CA | TYR | 2236 | 35.817 | 24.796 | −13.872 | 1.00 | 55.92 |
| ATOM | 4905 | CB | TYR | 2236 | 34.590 | 24.440 | −14.705 | 1.00 | 57.98 |
| ATOM | 4906 | CG | TYR | 2236 | 34.943 | 23.862 | −16.049 | 1.00 | 60.63 |
| ATOM | 4907 | CD1 | TYR | 2236 | 35.580 | 22.622 | −16.153 | 1.00 | 61.38 |
| ATOM | 4908 | CE1 | TYR | 2236 | 35.949 | 22.102 | −17.392 | 1.00 | 62.29 |
| ATOM | 4909 | CD2 | TYR | 2236 | 34.680 | 24.570 | −17.221 | 1.00 | 61.60 |
| ATOM | 4910 | CE2 | TYR | 2236 | 35.045 | 24.056 | −18.467 | 1.00 | 62.75 |
| ATOM | 4911 | CZ | TYR | 2236 | 35.679 | 22.825 | −18.545 | 1.00 | 62.44 |
| ATOM | 4912 | OH | TYR | 2236 | 36.039 | 22.325 | −19.775 | 1.00 | 63.37 |
| ATOM | 4913 | C | TYR | 2236 | 35.354 | 25.559 | −12.644 | 1.00 | 55.06 |
| ATOM | 4914 | O | TYR | 2236 | 34.705 | 26.598 | −12.775 | 1.00 | 55.01 |
| ATOM | 4915 | N | GLY | 2237 | 35.663 | 25.059 | −11.455 | 1.00 | 53.84 |
| ATOM | 4916 | CA | GLY | 2237 | 35.226 | 25.766 | −10.267 | 1.00 | 52.12 |
| ATOM | 4917 | C | GLY | 2237 | 35.282 | 24.947 | −8.999 | 1.00 | 50.95 |
| ATOM | 4918 | O | GLY | 2237 | 35.463 | 23.728 | −9.026 | 1.00 | 50.48 |
| ATOM | 4919 | N | SER | 2238 | 35.118 | 25.636 | −7.876 | 1.00 | 50.13 |
| ATOM | 4920 | CA | SER | 2238 | 35.166 | 24.999 | −6.571 | 1.00 | 48.85 |
| ATOM | 4921 | CB | SER | 2238 | 36.588 | 25.106 | −6.002 | 1.00 | 48.81 |
| ATOM | 4922 | OG | SER | 2238 | 36.670 | 24.575 | −4.690 | 1.00 | 49.44 |
| ATOM | 4923 | C | SER | 2238 | 34.181 | 25.623 | −5.589 | 1.00 | 47.60 |
| ATOM | 4924 | O | SER | 2238 | 34.068 | 26.845 | −5.497 | 1.00 | 46.99 |
| ATOM | 4925 | N | ILE | 2239 | 33.461 | 24.771 | −4.869 | 1.00 | 45.96 |
| ATOM | 4926 | CA | ILE | 2239 | 32.526 | 25.237 | −3.863 | 1.00 | 44.63 |
| ATOM | 4927 | CB | ILE | 2239 | 31.070 | 24.941 | −4.232 | 1.00 | 44.34 |
| ATOM | 4928 | CG2 | ILE | 2239 | 30.685 | 25.762 | −5.448 | 1.00 | 44.32 |
| ATOM | 4929 | CG1 | ILE | 2239 | 30.867 | 23.447 | −4.468 | 1.00 | 43.70 |
| ATOM | 4930 | CD1 | ILE | 2239 | 29.433 | 23.095 | −4.841 | 1.00 | 42.21 |
| ATOM | 4931 | C | ILE | 2239 | 32.864 | 24.554 | −2.551 | 1.00 | 44.25 |
| ATOM | 4932 | O | ILE | 2239 | 33.665 | 23.616 | −2.520 | 1.00 | 44.20 |
| ATOM | 4933 | N | ASN | 2240 | 32.272 | 25.034 | −1.466 | 1.00 | 43.32 |
| ATOM | 4934 | CA | ASN | 2240 | 32.543 | 24.451 | −0.165 | 1.00 | 42.74 |
| ATOM | 4935 | CB | ASN | 2240 | 33.924 | 24.891 | 0.345 | 1.00 | 42.39 |
| ATOM | 4936 | CG | ASN | 2240 | 33.993 | 26.372 | 0.662 | 1.00 | 42.39 |
| ATOM | 4937 | OD1 | ASN | 2240 | 33.242 | 26.879 | 1.495 | 1.00 | 42.55 |
| ATOM | 4938 | ND2 | ASN | 2240 | 34.905 | 27.072 | 0.005 | 1.00 | 42.59 |
| ATOM | 4939 | C | ASN | 2240 | 31.471 | 24.788 | 0.862 | 1.00 | 42.27 |
| ATOM | 4940 | O | ASN | 2240 | 30.674 | 25.707 | 0.676 | 1.00 | 41.96 |
| ATOM | 4941 | N | HIS | 2241 | 31.462 | 24.020 | 1.946 | 1.00 | 41.58 |
| ATOM | 4942 | CA | HIS | 2241 | 30.494 | 24.197 | 3.016 | 1.00 | 40.51 |
| ATOM | 4943 | CB | HIS | 2241 | 29.277 | 23.316 | 2.755 | 1.00 | 41.07 |
| ATOM | 4944 | CG | HIS | 2241 | 28.072 | 23.688 | 3.560 | 1.00 | 42.82 |
| ATOM | 4945 | CD2 | HIS | 2241 | 27.393 | 23.007 | 4.513 | 1.00 | 43.30 |
| ATOM | 4946 | ND1 | HIS | 2241 | 27.392 | 24.873 | 3.377 | 1.00 | 42.91 |
| ATOM | 4947 | CE1 | HIS | 2241 | 26.342 | 24.904 | 4.179 | 1.00 | 43.30 |
| ATOM | 4948 | NE2 | HIS | 2241 | 26.319 | 23.783 | 4.878 | 1.00 | 43.85 |
| ATOM | 4949 | C | HIS | 2241 | 31.179 | 23.757 | 4.296 | 1.00 | 39.54 |
| ATOM | 4950 | O | HIS | 2241 | 32.035 | 22.872 | 4.273 | 1.00 | 39.23 |
| ATOM | 4951 | N | THR | 2242 | 30.819 | 24.379 | 5.412 | 1.00 | 38.52 |
| ATOM | 4952 | CA | THR | 2242 | 31.420 | 24.013 | 6.683 | 1.00 | 37.22 |
| ATOM | 4953 | CB | THR | 2242 | 32.370 | 25.111 | 7.212 | 1.00 | 36.83 |
| ATOM | 4954 | OG1 | THR | 2242 | 33.419 | 25.329 | 6.265 | 1.00 | 36.71 |
| ATOM | 4955 | CG2 | THR | 2242 | 33.007 | 24.673 | 8.541 | 1.00 | 36.35 |
| ATOM | 4956 | C | THR | 2242 | 30.400 | 23.692 | 7.762 | 1.00 | 36.30 |
| ATOM | 4957 | O | THR | 2242 | 29.454 | 24.438 | 7.992 | 1.00 | 37.17 |
| ATOM | 4958 | N | TYR | 2243 | 30.604 | 22.561 | 8.415 | 1.00 | 35.08 |
| ATOM | 4959 | CA | TYR | 2243 | 29.733 | 22.120 | 9.485 | 1.00 | 34.47 |
| ATOM | 4960 | CB | TYR | 2243 | 29.341 | 20.663 | 9.286 | 1.00 | 33.92 |
| ATOM | 4961 | CG | TYR | 2243 | 28.512 | 20.404 | 8.053 | 1.00 | 34.46 |
| ATOM | 4962 | CD1 | TYR | 2243 | 29.089 | 19.888 | 6.889 | 1.00 | 34.20 |
| ATOM | 4963 | CE1 | TYR | 2243 | 28.310 | 19.562 | 5.785 | 1.00 | 33.91 |
| ATOM | 4964 | CD2 | TYR | 2243 | 27.131 | 20.602 | 8.071 | 1.00 | 34.23 |
| ATOM | 4965 | CE2 | TYR | 2243 | 26.343 | 20.281 | 6.972 | 1.00 | 34.24 |
| ATOM | 4966 | CZ | TYR | 2243 | 26.935 | 19.756 | 5.833 | 1.00 | 34.30 |
| ATOM | 4967 | OH | TYR | 2243 | 26.143 | 19.399 | 4.766 | 1.00 | 33.85 |
| ATOM | 4968 | C | TYR | 2243 | 30.482 | 22.236 | 10.795 | 1.00 | 34.34 |
| ATOM | 4969 | O | TYR | 2243 | 31.709 | 22.108 | 10.835 | 1.00 | 34.23 |
| ATOM | 4970 | N | GLN | 2244 | 29.762 | 22.504 | 11.874 | 1.00 | 34.45 |
| ATOM | 4971 | CA | GLN | 2244 | 30.433 | 22.570 | 13.152 | 1.00 | 34.76 |
| ATOM | 4972 | CB | GLN | 2244 | 30.092 | 23.835 | 13.926 | 1.00 | 36.17 |
| ATOM | 4973 | CG | GLN | 2244 | 30.753 | 23.825 | 15.300 | 1.00 | 39.01 |
| ATOM | 4974 | CD | GLN | 2244 | 30.668 | 25.149 | 16.031 | 1.00 | 40.93 |
| ATOM | 4975 | OE1 | GLN | 2244 | 29.824 | 26.000 | 15.724 | 1.00 | 42.17 |
| ATOM | 4976 | NE2 | GLN | 2244 | 31.535 | 25.322 | 17.021 | 1.00 | 41.16 |
| ATOM | 4977 | C | GLN | 2244 | 30.025 | 21.355 | 13.951 | 1.00 | 33.80 |
| ATOM | 4978 | O | GLN | 2244 | 28.840 | 21.033 | 14.052 | 1.00 | 34.03 |
| ATOM | 4979 | N | LEU | 2245 | 31.021 | 20.663 | 14.491 | 1.00 | 32.38 |
| ATOM | 4980 | CA | LEU | 2245 | 30.771 | 19.480 | 15.294 | 1.00 | 31.36 |
| ATOM | 4981 | CB | LEU | 2245 | 31.676 | 18.332 | 14.868 | 1.00 | 30.16 |
| ATOM | 4982 | CG | LEU | 2245 | 31.473 | 17.087 | 15.730 | 1.00 | 29.54 |
| ATOM | 4983 | CD1 | LEU | 2245 | 30.015 | 16.718 | 15.724 | 1.00 | 29.57 |
| ATOM | 4984 | CD2 | LEU | 2245 | 32.315 | 15.943 | 15.217 | 1.00 | 30.03 |
| ATOM | 4985 | C | LEU | 2245 | 31.017 | 19.766 | 16.758 | 1.00 | 31.10 |
| ATOM | 4986 | O | LEU | 2245 | 32.083 | 20.232 | 17.143 | 1.00 | 31.15 |
| ATOM | 4987 | N | ASP | 2246 | 30.018 | 19.487 | 17.576 | 1.00 | 31.64 |
| ATOM | 4988 | CA | ASP | 2246 | 30.136 | 19.690 | 19.006 | 1.00 | 32.13 |
| ATOM | 4989 | CB | ASP | 2246 | 29.198 | 20.801 | 19.439 | 1.00 | 33.14 |
| ATOM | 4990 | CG | ASP | 2246 | 29.462 | 21.247 | 20.841 | 1.00 | 34.62 |
| ATOM | 4991 | OD1 | ASP | 2246 | 28.931 | 22.309 | 21.220 | 1.00 | 35.69 |
| ATOM | 4992 | OD2 | ASP | 2246 | 30.199 | 20.532 | 21.562 | 1.00 | 35.10 |
| ATOM | 4993 | C | ASP | 2246 | 29.776 | 18.386 | 19.707 | 1.00 | 32.20 |
| ATOM | 4994 | O | ASP | 2246 | 28.711 | 17.810 | 19.457 | 1.00 | 33.32 |
| ATOM | 4995 | N | VAL | 2247 | 30.669 | 17.902 | 20.560 | 1.00 | 31.15 |
| ATOM | 4996 | CA | VAL | 2247 | 30.419 | 16.660 | 21.269 | 1.00 | 30.49 |
| ATOM | 4997 | CB | VAL | 2247 | 31.551 | 15.667 | 21.036 | 1.00 | 29.19 |
| ATOM | 4998 | CG1 | VAL | 2247 | 31.249 | 14.358 | 21.740 | 1.00 | 28.05 |
| ATOM | 4999 | CG2 | VAL | 2247 | 31.726 | 15.460 | 19.548 | 1.00 | 27.71 |
| ATOM | 5000 | C | VAL | 2247 | 30.261 | 16.966 | 22.745 | 1.00 | 31.10 |
| ATOM | 5001 | O | VAL | 2247 | 31.083 | 17.645 | 23.339 | 1.00 | 32.18 |
| ATOM | 5002 | N | VAL | 2248 | 29.180 | 16.466 | 23.326 | 1.00 | 31.45 |
| ATOM | 5003 | CA | VAL | 2248 | 28.836 | 16.701 | 24.733 | 1.00 | 31.22 |
| ATOM | 5004 | CB | VAL | 2248 | 27.366 | 17.220 | 24.831 | 1.00 | 30.59 |
| ATOM | 5005 | CG1 | VAL | 2248 | 26.958 | 17.377 | 26.270 | 1.00 | 30.54 |
| ATOM | 5006 | CG2 | VAL | 2248 | 27.216 | 18.535 | 24.077 | 1.00 | 29.87 |
| ATOM | 5007 | C | VAL | 2248 | 28.919 | 15.402 | 25.545 | 1.00 | 31.25 |
| ATOM | 5008 | O | VAL | 2248 | 28.308 | 14.415 | 25.179 | 1.00 | 31.16 |
| ATOM | 5009 | N | GLU | 2249 | 29.631 | 15.363 | 26.656 | 1.00 | 31.21 |
| ATOM | 5010 | CA | GLU | 2249 | 29.670 | 14.095 | 27.390 | 1.00 | 31.91 |
| ATOM | 5011 | CB | GLU | 2249 | 31.043 | 13.916 | 28.040 | 1.00 | 32.28 |
| ATOM | 5012 | CG | GLU | 2249 | 32.153 | 14.052 | 27.021 | 1.00 | 34.76 |
| ATOM | 5013 | CD | GLU | 2249 | 33.485 | 13.495 | 27.483 | 1.00 | 36.49 |
| ATOM | 5014 | OE1 | GLU | 2249 | 34.108 | 14.083 | 28.399 | 1.00 | 36.07 |
| ATOM | 5015 | OE2 | GLU | 2249 | 33.905 | 12.459 | 26.913 | 1.00 | 37.06 |
| ATOM | 5016 | C | GLU | 2249 | 28.540 | 14.009 | 28.423 | 1.00 | 31.23 |
| ATOM | 5017 | O | GLU | 2249 | 28.219 | 15.004 | 29.073 | 1.00 | 31.78 |
| ATOM | 5018 | N | ARG | 2250 | 27.926 | 12.832 | 28.555 | 1.00 | 29.85 |
| ATOM | 5019 | CA | ARG | 2250 | 26.816 | 12.640 | 29.501 | 1.00 | 29.42 |
| ATOM | 5020 | CB | ARG | 2250 | 25.643 | 11.906 | 28.809 | 1.00 | 28.75 |
| ATOM | 5021 | CG | ARG | 2250 | 25.106 | 12.572 | 27.524 | 1.00 | 26.64 |
| ATOM | 5022 | CD | ARG | 2250 | 24.761 | 14.041 | 27.732 | 1.00 | 25.25 |
| ATOM | 5023 | NE | ARG | 2250 | 23.792 | 14.248 | 28.808 | 1.00 | 26.29 |
| ATOM | 5024 | CZ | ARG | 2250 | 22.467 | 14.222 | 28.668 | 1.00 | 25.22 |
| ATOM | 5025 | NH1 | ARG | 2250 | 21.912 | 14.004 | 27.490 | 1.00 | 25.69 |
| ATOM | 5026 | NH2 | ARG | 2250 | 21.693 | 14.406 | 29.720 | 1.00 | 25.76 |
| ATOM | 5027 | C | ARG | 2250 | 27.253 | 11.855 | 30.747 | 1.00 | 29.20 |
| ATOM | 5028 | O | ARG | 2250 | 28.274 | 11.179 | 30.719 | 1.00 | 28.85 |
| ATOM | 5029 | N | SER | 2251 | 26.487 | 11.951 | 31.834 | 1.00 | 29.62 |
| ATOM | 5030 | CA | SER | 2251 | 26.813 | 11.237 | 33.073 | 1.00 | 30.87 |
| ATOM | 5031 | CB | SER | 2251 | 27.559 | 12.138 | 34.056 | 1.00 | 31.32 |
| ATOM | 5032 | OG | SER | 2251 | 28.741 | 12.660 | 33.481 | 1.00 | 33.17 |
| ATOM | 5033 | C | SER | 2251 | 25.563 | 10.744 | 33.758 | 1.00 | 31.52 |
| ATOM | 5034 | O | SER | 2251 | 24.962 | 11.466 | 34.545 | 1.00 | 32.06 |
| ATOM | 5035 | N | PRO | 2252 | 25.161 | 9.496 | 33.481 | 1.00 | 32.23 |
| ATOM | 5036 | CD | PRO | 2252 | 25.723 | 8.595 | 32.457 | 1.00 | 32.11 |
| ATOM | 5037 | CA | PRO | 2252 | 23.964 | 8.910 | 34.081 | 1.00 | 32.83 |
| ATOM | 5038 | CB | PRO | 2252 | 23.558 | 7.866 | 33.049 | 1.00 | 32.65 |
| ATOM | 5039 | CG | PRO | 2252 | 24.872 | 7.344 | 32.609 | 1.00 | 32.26 |
| ATOM | 5040 | C | PRO | 2252 | 24.220 | 8.301 | 35.455 | 1.00 | 33.52 |
| ATOM | 5041 | O | PRO | 2252 | 24.166 | 7.075 | 35.619 | 1.00 | 34.55 |
| ATOM | 5042 | N | HIS | 2253 | 24.495 | 9.158 | 36.437 | 1.00 | 33.82 |
| ATOM | 5043 | CA | HIS | 2253 | 24.750 | 8.730 | 37.804 | 1.00 | 33.87 |
| ATOM | 5044 | CB | HIS | 2253 | 26.262 | 8.764 | 38.057 | 1.00 | 36.50 |
| ATOM | 5045 | CG | HIS | 2253 | 27.014 | 7.657 | 37.377 | 1.00 | 40.62 |
| ATOM | 5046 | CD2 | HIS | 2253 | 27.986 | 6.828 | 37.832 | 1.00 | 42.22 |
| ATOM | 5047 | ND1 | HIS | 2253 | 26.798 | 7.300 | 36.061 | 1.00 | 42.32 |
| ATOM | 5048 | CE1 | HIS | 2253 | 27.601 | 6.303 | 35.734 | 1.00 | 42.14 |
| ATOM | 5049 | NE2 | HIS | 2253 | 28.334 | 5.997 | 36.791 | 1.00 | 42.84 |
| ATOM | 5050 | C | HIS | 2253 | 24.016 | 9.656 | 38.768 | 1.00 | 32.55 |
| ATOM | 5051 | O | HIS | 2253 | 23.611 | 10.750 | 38.385 | 1.00 | 32.05 |
| ATOM | 5052 | N | ARG | 2254 | 23.830 | 9.211 | 40.006 | 1.00 | 31.63 |
| ATOM | 5053 | CA | ARG | 2254 | 23.165 | 10.030 | 41.012 | 1.00 | 30.71 |
| ATOM | 5054 | CB | ARG | 2254 | 23.038 | 9.241 | 42.317 | 1.00 | 31.85 |
| ATOM | 5055 | CG | ARG | 2254 | 24.353 | 8.691 | 42.836 | 1.00 | 32.46 |
| ATOM | 5056 | CD | ARG | 2254 | 24.123 | 7.624 | 43.886 | 1.00 | 35.54 |
| ATOM | 5057 | NE | ARG | 2254 | 24.523 | 8.058 | 45.227 | 1.00 | 39.43 |
| ATOM | 5058 | CZ | ARG | 2254 | 25.693 | 7.778 | 45.806 | 1.00 | 39.98 |
| ATOM | 5059 | NH1 | ARG | 2254 | 26.608 | 7.049 | 45.167 | 1.00 | 39.96 |
| ATOM | 5060 | NH2 | ARG | 2254 | 25.951 | 8.235 | 47.027 | 1.00 | 39.85 |
| ATOM | 5061 | C | ARG | 2254 | 24.030 | 11.278 | 41.222 | 1.00 | 29.81 |
| ATOM | 5062 | O | ARG | 2254 | 25.209 | 11.300 | 40.840 | 1.00 | 28.68 |
| ATOM | 5063 | N | PRO | 2255 | 23.462 | 12.338 | 41.818 | 1.00 | 28.57 |
| ATOM | 5064 | CD | PRO | 2255 | 22.051 | 12.624 | 42.114 | 1.00 | 27.59 |
| ATOM | 5065 | CA | PRO | 2255 | 24.299 | 13.524 | 42.013 | 1.00 | 27.57 |
| ATOM | 5066 | CB | PRO | 2255 | 23.314 | 14.569 | 42.546 | 1.00 | 27.22 |
| ATOM | 5067 | CG | PRO | 2255 | 22.165 | 13.773 | 43.049 | 1.00 | 27.56 |
| ATOM | 5068 | C | PRO | 2255 | 25.508 | 13.265 | 42.920 | 1.00 | 27.48 |
| ATOM | 5069 | O | PRO | 2255 | 25.505 | 12.358 | 43.754 | 1.00 | 26.80 |
| ATOM | 5070 | N | ILE | 2256 | 26.553 | 14.056 | 42.712 | 1.00 | 27.43 |
| ATOM | 5071 | CA | ILE | 2256 | 27.795 | 13.944 | 43.460 | 1.00 | 26.43 |
| ATOM | 5072 | CB | ILE | 2256 | 28.990 | 13.768 | 42.499 | 1.00 | 25.95 |
| ATOM | 5073 | CG2 | ILE | 2256 | 30.286 | 13.620 | 43.279 | 1.00 | 23.53 |
| ATOM | 5074 | CG1 | ILE | 2256 | 28.764 | 12.549 | 41.613 | 1.00 | 25.30 |
| ATOM | 5075 | CD1 | ILE | 2256 | 29.792 | 12.403 | 40.502 | 1.00 | 25.01 |
| ATOM | 5076 | C | ILE | 2256 | 28.012 | 15.229 | 44.240 | 1.00 | 26.92 |
| ATOM | 5077 | O | ILE | 2256 | 27.953 | 16.313 | 43.668 | 1.00 | 27.24 |
| ATOM | 5078 | N | LEU | 2257 | 28.266 | 15.103 | 45.538 | 1.00 | 26.99 |
| ATOM | 5079 | CA | LEU | 2257 | 28.502 | 16.255 | 46.386 | 1.00 | 27.39 |
| ATOM | 5080 | CB | LEU | 2257 | 27.762 | 16.070 | 47.711 | 1.00 | 27.19 |
| ATOM | 5081 | CG | LEU | 2257 | 26.294 | 15.594 | 47.689 | 1.00 | 28.23 |
| ATOM | 5082 | CD1 | LEU | 2257 | 25.668 | 15.909 | 49.040 | 1.00 | 29.07 |
| ATOM | 5083 | CD2 | LEU | 2257 | 25.479 | 16.270 | 46.610 | 1.00 | 27.48 |
| ATOM | 5084 | C | LEU | 2257 | 30.004 | 16.387 | 46.628 | 1.00 | 28.16 |
| ATOM | 5085 | O | LEU | 2257 | 30.675 | 15.389 | 46.867 | 1.00 | 28.15 |
| ATOM | 5086 | N | GLN | 2258 | 30.548 | 17.602 | 46.556 | 1.00 | 28.83 |
| ATOM | 5087 | CA | GLN | 2258 | 31.986 | 17.772 | 46.776 | 1.00 | 29.34 |
| ATOM | 5088 | CB | GLN | 2258 | 32.445 | 19.212 | 46.520 | 1.00 | 29.65 |
| ATOM | 5089 | CG | GLN | 2258 | 33.911 | 19.459 | 46.960 | 1.00 | 30.01 |
| ATOM | 5090 | CD | GLN | 2258 | 34.936 | 18.564 | 46.237 | 1.00 | 31.16 |
| ATOM | 5091 | OE1 | GLN | 2258 | 34.603 | 17.488 | 45.704 | 1.00 | 30.77 |
| ATOM | 5092 | NE2 | GLN | 2258 | 36.190 | 19.001 | 46.235 | 1.00 | 30.89 |
| ATOM | 5093 | C | GLN | 2258 | 32.402 | 17.376 | 48.185 | 1.00 | 29.48 |
| ATOM | 5094 | O | GLN | 2258 | 31.857 | 17.857 | 49.171 | 1.00 | 30.23 |
| ATOM | 5095 | N | ALA | 2259 | 33.384 | 16.494 | 48.269 | 1.00 | 29.13 |
| ATOM | 5096 | CA | ALA | 2259 | 33.872 | 16.046 | 49.553 | 1.00 | 29.42 |
| ATOM | 5097 | CB | ALA | 2259 | 35.121 | 15.214 | 49.368 | 1.00 | 30.08 |
| ATOM | 5098 | C | ALA | 2259 | 34.181 | 17.231 | 50.458 | 1.00 | 29.94 |
| ATOM | 5099 | O | ALA | 2259 | 34.668 | 18.259 | 50.004 | 1.00 | 30.39 |
| ATOM | 5100 | N | GLY | 2260 | 33.896 | 17.092 | 51.746 | 1.00 | 30.53 |
| ATOM | 5101 | CA | GLY | 2260 | 34.199 | 18.167 | 52.674 | 1.00 | 30.54 |
| ATOM | 5102 | C | GLY | 2260 | 33.118 | 19.197 | 52.938 | 1.00 | 31.12 |
| ATOM | 5103 | O | GLY | 2260 | 33.215 | 19.932 | 53.920 | 1.00 | 31.99 |
| ATOM | 5104 | N | LEU | 2261 | 32.095 | 19.265 | 52.091 | 1.00 | 30.98 |
| ATOM | 5105 | CA | LEU | 2261 | 31.033 | 20.243 | 52.270 | 1.00 | 30.63 |
| ATOM | 5106 | CB | LEU | 2261 | 31.031 | 21.208 | 51.078 | 1.00 | 30.72 |
| ATOM | 5107 | CG | LEU | 2261 | 32.365 | 21.933 | 50.824 | 1.00 | 31.66 |
| ATOM | 5108 | CD1 | LEU | 2261 | 32.294 | 22.731 | 49.530 | 1.00 | 31.02 |
| ATOM | 5109 | CD2 | LEU | 2261 | 32.683 | 22.850 | 51.995 | 1.00 | 31.07 |
| ATOM | 5110 | C | LEU | 2261 | 29.660 | 19.589 | 52.407 | 1.00 | 30.64 |
| ATOM | 5111 | O | LEU | 2261 | 29.338 | 18.665 | 51.664 | 1.00 | 30.81 |
| ATOM | 5112 | N | PRO | 2262 | 28.824 | 20.070 | 53.356 | 1.00 | 30.49 |
| ATOM | 5113 | CD | PRO | 2262 | 27.462 | 19.546 | 53.578 | 1.00 | 29.90 |
| ATOM | 5114 | CA | PRO | 2262 | 29.113 | 21.177 | 54.281 | 1.00 | 30.55 |
| ATOM | 5115 | CB | PRO | 2262 | 27.744 | 21.488 | 54.879 | 1.00 | 30.32 |
| ATOM | 5116 | CG | PRO | 2262 | 27.106 | 20.123 | 54.934 | 1.00 | 30.38 |
| ATOM | 5117 | C | PRO | 2262 | 30.124 | 20.744 | 55.334 | 1.00 | 30.64 |
| ATOM | 5118 | O | PRO | 2262 | 30.319 | 19.556 | 55.551 | 1.00 | 31.68 |
| ATOM | 5119 | N | ALA | 2263 | 30.764 | 21.702 | 55.987 | 1.00 | 30.86 |
| ATOM | 5120 | CA | ALA | 2263 | 31.758 | 21.382 | 57.006 | 1.00 | 31.01 |
| ATOM | 5121 | CB | ALA | 2263 | 33.052 | 22.118 | 56.716 | 1.00 | 31.24 |
| ATOM | 5122 | C | ALA | 2263 | 31.290 | 21.704 | 58.415 | 1.00 | 31.05 |
| ATOM | 5123 | O | ALA | 2263 | 30.459 | 22.584 | 58.624 | 1.00 | 30.68 |
| ATOM | 5124 | N | ASN | 2264 | 31.825 | 20.970 | 59.383 | 1.00 | 32.10 |
| ATOM | 5125 | CA | ASN | 2264 | 31.481 | 21.177 | 60.786 | 1.00 | 33.19 |
| ATOM | 5126 | CB | ASN | 2264 | 32.234 | 20.180 | 61.668 | 1.00 | 31.52 |
| ATOM | 5127 | CG | ASN | 2264 | 31.724 | 18.763 | 61.518 | 1.00 | 30.77 |
| ATOM | 5128 | OD1 | ASN | 2264 | 30.576 | 18.546 | 61.137 | 1.00 | 31.16 |
| ATOM | 5129 | ND2 | ASN | 2264 | 32.561 | 17.790 | 61.848 | 1.00 | 29.76 |
| ATOM | 5130 | C | ASN | 2264 | 31.856 | 22.598 | 61.192 | 1.00 | 34.67 |
| ATOM | 5131 | O | ASN | 2264 | 32.882 | 23.119 | 60.767 | 1.00 | 35.43 |
| ATOM | 5132 | N | LYS | 2265 | 31.032 | 23.226 | 62.018 | 1.00 | 35.97 |
| ATOM | 5133 | CA | LYS | 2265 | 31.315 | 24.585 | 62.452 | 1.00 | 37.56 |
| ATOM | 5134 | CB | LYS | 2265 | 30.439 | 25.588 | 61.686 | 1.00 | 38.71 |
| ATOM | 5135 | CG | LYS | 2265 | 30.497 | 25.500 | 60.173 | 1.00 | 39.82 |
| ATOM | 5136 | CD | LYS | 2265 | 31.641 | 26.331 | 59.598 | 1.00 | 41.58 |
| ATOM | 5137 | CE | LYS | 2265 | 31.681 | 26.237 | 58.074 | 1.00 | 41.23 |
| ATOM | 5138 | NZ | LYS | 2265 | 30.368 | 26.625 | 57.474 | 1.00 | 42.04 |
| ATOM | 5139 | C | LYS | 2265 | 31.054 | 24.764 | 63.944 | 1.00 | 38.13 |
| ATOM | 5140 | O | LYS | 2265 | 30.080 | 24.250 | 64.489 | 1.00 | 37.80 |
| ATOM | 5141 | N | THR | 2266 | 31.945 | 25.493 | 64.601 | 1.00 | 39.03 |
| ATOM | 5142 | CA | THR | 2266 | 31.787 | 25.793 | 66.013 | 1.00 | 39.66 |
| ATOM | 5143 | CB | THR | 2266 | 32.954 | 25.268 | 66.849 | 1.00 | 39.06 |
| ATOM | 5144 | OG1 | THR | 2266 | 33.083 | 23.861 | 66.635 | 1.00 | 39.27 |
| ATOM | 5145 | CG2 | THR | 2266 | 32.699 | 25.517 | 68.335 | 1.00 | 38.84 |
| ATOM | 5146 | C | THR | 2266 | 31.745 | 27.313 | 66.073 | 1.00 | 39.78 |
| ATOM | 5147 | O | THR | 2266 | 32.698 | 27.983 | 65.695 | 1.00 | 39.92 |
| ATOM | 5148 | N | VAL | 2267 | 30.626 | 27.858 | 66.527 | 1.00 | 39.68 |
| ATOM | 5149 | CA | VAL | 2267 | 30.491 | 29.298 | 66.586 | 1.00 | 40.35 |
| ATOM | 5150 | CB | VAL | 2267 | 29.612 | 29.806 | 65.434 | 1.00 | 39.62 |
| ATOM | 5151 | CG1 | VAL | 2267 | 30.312 | 29.572 | 64.109 | 1.00 | 39.00 |
| ATOM | 5152 | CG2 | VAL | 2267 | 28.282 | 29.093 | 65.458 | 1.00 | 38.47 |
| ATOM | 5153 | C | VAL | 2267 | 29.913 | 29.801 | 67.896 | 1.00 | 41.43 |
| ATOM | 5154 | O | VAL | 2267 | 29.282 | 29.060 | 68.651 | 1.00 | 40.66 |
| ATOM | 5155 | N | ALA | 2268 | 30.136 | 31.083 | 68.153 | 1.00 | 42.97 |
| ATOM | 5156 | CA | ALA | 2268 | 29.652 | 31.712 | 69.366 | 1.00 | 44.16 |
| ATOM | 5157 | CB | ALA | 2268 | 30.455 | 32.983 | 69.646 | 1.00 | 44.30 |
| ATOM | 5158 | C | ALA | 2268 | 28.175 | 32.038 | 69.214 | 1.00 | 44.84 |
| ATOM | 5159 | O | ALA | 2268 | 27.695 | 32.318 | 68.115 | 1.00 | 44.50 |
| ATOM | 5160 | N | LEU | 2269 | 27.453 | 31.994 | 70.325 | 1.00 | 45.61 |
| ATOM | 5161 | CA | LEU | 2269 | 26.028 | 32.287 | 70.307 | 1.00 | 46.16 |
| ATOM | 5162 | CB | LEU | 2269 | 25.493 | 32.339 | 71.738 | 1.00 | 45.87 |
| ATOM | 5163 | CG | LEU | 2269 | 24.209 | 31.561 | 72.033 | 1.00 | 46.18 |
| ATOM | 5164 | CD1 | LEU | 2269 | 24.035 | 31.419 | 73.540 | 1.00 | 46.48 |
| ATOM | 5165 | CD2 | LEU | 2269 | 23.019 | 32.268 | 71.407 | 1.00 | 46.06 |
| ATOM | 5166 | C | LEU | 2269 | 25.808 | 33.629 | 69.618 | 1.00 | 46.93 |
| ATOM | 5167 | O | LEU | 2269 | 26.605 | 34.557 | 69.780 | 1.00 | 47.56 |
| ATOM | 5168 | N | GLY | 2270 | 24.735 | 33.726 | 68.841 | 1.00 | 47.09 |
| ATOM | 5169 | CA | GLY | 2270 | 24.434 | 34.969 | 68.155 | 1.00 | 47.38 |
| ATOM | 5170 | C | GLY | 2270 | 25.197 | 35.209 | 66.864 | 1.00 | 47.78 |
| ATOM | 5171 | O | GLY | 2270 | 24.988 | 36.229 | 66.210 | 1.00 | 47.72 |
| ATOM | 5172 | N | SER | 2271 | 26.080 | 34.285 | 66.492 | 1.00 | 47.62 |
| ATOM | 5173 | CA | SER | 2271 | 26.853 | 34.429 | 65.261 | 1.00 | 47.44 |
| ATOM | 5174 | CB | SER | 2271 | 28.051 | 33.476 | 65.256 | 1.00 | 47.64 |
| ATOM | 5175 | OG | SER | 2271 | 28.835 | 33.613 | 66.426 | 1.00 | 49.57 |
| ATOM | 5176 | C | SER | 2271 | 25.996 | 34.116 | 64.039 | 1.00 | 47.34 |
| ATOM | 5177 | O | SER | 2271 | 24.871 | 33.616 | 64.151 | 1.00 | 47.41 |
| ATOM | 5178 | N | ASN | 2272 | 26.535 | 34.429 | 62.869 | 1.00 | 46.71 |
| ATOM | 5179 | CA | ASN | 2272 | 25.858 | 34.129 | 61.622 | 1.00 | 46.45 |
| ATOM | 5180 | CB | ASN | 2272 | 25.808 | 35.348 | 60.705 | 1.00 | 46.03 |
| ATOM | 5181 | CG | ASN | 2272 | 24.886 | 36.423 | 61.222 | 1.00 | 46.41 |
| ATOM | 5182 | OD1 | ASN | 2272 | 23.742 | 36.154 | 61.590 | 1.00 | 45.55 |
| ATOM | 5183 | ND2 | ASN | 2272 | 25.376 | 37.653 | 61.247 | 1.00 | 46.88 |
| ATOM | 5184 | C | ASN | 2272 | 26.710 | 33.040 | 60.995 | 1.00 | 46.48 |
| ATOM | 5185 | O | ASN | 2272 | 27.938 | 33.113 | 61.026 | 1.00 | 46.62 |
| ATOM | 5186 | N | VAL | 2273 | 26.072 | 32.021 | 60.437 | 1.00 | 45.88 |
| ATOM | 5187 | CA | VAL | 2273 | 26.820 | 30.929 | 59.838 | 1.00 | 45.34 |
| ATOM | 5188 | CB | VAL | 2273 | 26.857 | 29.697 | 60.792 | 1.00 | 46.10 |
| ATOM | 5189 | CG1 | VAL | 2273 | 25.496 | 29.510 | 61.449 | 1.00 | 46.20 |
| ATOM | 5190 | CG2 | VAL | 2273 | 27.228 | 28.429 | 60.016 | 1.00 | 46.19 |
| ATOM | 5191 | C | VAL | 2273 | 26.223 | 30.500 | 58.520 | 1.00 | 44.35 |
| ATOM | 5192 | O | VAL | 2273 | 25.009 | 30.559 | 58.335 | 1.00 | 44.73 |
| ATOM | 5193 | N | GLU | 2274 | 27.079 | 30.071 | 57.602 | 1.00 | 43.27 |
| ATOM | 5194 | CA | GLU | 2274 | 26.607 | 29.601 | 56.314 | 1.00 | 42.55 |
| ATOM | 5195 | CB | GLU | 2274 | 26.870 | 30.637 | 55.206 | 1.00 | 43.72 |
| ATOM | 5196 | CG | GLU | 2274 | 28.331 | 30.930 | 54.860 | 1.00 | 44.53 |
| ATOM | 5197 | CD | GLU | 2274 | 28.474 | 32.021 | 53.777 | 1.00 | 45.94 |
| ATOM | 5198 | OE1 | GLU | 2274 | 27.924 | 33.137 | 53.960 | 1.00 | 45.53 |
| ATOM | 5199 | OE2 | GLU | 2274 | 29.138 | 31.767 | 52.744 | 1.00 | 45.60 |
| ATOM | 5200 | C | GLU | 2274 | 27.227 | 28.266 | 55.941 | 1.00 | 41.35 |
| ATOM | 5201 | O | GLU | 2274 | 28.446 | 28.113 | 55.910 | 1.00 | 40.94 |
| ATOM | 5202 | N | PHE | 2275 | 26.366 | 27.290 | 55.687 | 1.00 | 39.80 |
| ATOM | 5203 | CA | PHE | 2275 | 26.809 | 25.970 | 55.289 | 1.00 | 38.16 |
| ATOM | 5204 | CB | PHE | 2275 | 25.857 | 24.908 | 55.825 | 1.00 | 36.24 |
| ATOM | 5205 | CG | PHE | 2275 | 26.079 | 24.569 | 57.269 | 1.00 | 34.62 |
| ATOM | 5206 | CD1 | PHE | 2275 | 27.289 | 24.037 | 57.688 | 1.00 | 33.49 |
| ATOM | 5207 | CD2 | PHE | 2275 | 25.065 | 24.734 | 58.201 | 1.00 | 34.11 |
| ATOM | 5208 | CE1 | PHE | 2275 | 27.487 | 23.669 | 59.004 | 1.00 | 33.95 |
| ATOM | 5209 | CE2 | PHE | 2275 | 25.256 | 24.366 | 59.527 | 1.00 | 33.81 |
| ATOM | 5210 | CZ | PHE | 2275 | 26.471 | 23.829 | 59.930 | 1.00 | 33.81 |
| ATOM | 5211 | C | PHE | 2275 | 26.800 | 25.946 | 53.781 | 1.00 | 38.28 |
| ATOM | 5212 | O | PHE | 2275 | 25.927 | 26.547 | 53.153 | 1.00 | 38.22 |
| ATOM | 5213 | N | MSE | 2276 | 27.775 | 25.266 | 53.195 | 1.00 | 38.84 |
| ATOM | 5214 | CA | MSE | 2276 | 27.837 | 25.179 | 51.750 | 1.00 | 39.52 |
| ATOM | 5215 | CB | MSE | 2276 | 29.113 | 25.825 | 51.236 | 1.00 | 41.46 |
| ATOM | 5216 | CG | MSE | 2276 | 28.914 | 27.271 | 50.865 | 1.00 | 45.12 |
| ATOM | 5217 | SE | MSE | 2276 | 30.481 | 28.078 | 50.709 | 1.00 | 50.12 |
| ATOM | 5218 | CE | MSE | 2276 | 31.138 | 27.258 | 49.210 | 1.00 | 48.16 |
| ATOM | 5219 | C | MSE | 2276 | 27.726 | 23.759 | 51.237 | 1.00 | 38.31 |
| ATOM | 5220 | O | MSE | 2276 | 27.938 | 22.803 | 51.972 | 1.00 | 37.11 |
| ATOM | 5221 | N | CYS | 2277 | 27.378 | 23.641 | 49.964 | 1.00 | 37.59 |
| ATOM | 5222 | CA | CYS | 2277 | 27.235 | 22.352 | 49.339 | 1.00 | 37.00 |
| ATOM | 5223 | CB | CYS | 2277 | 25.879 | 21.776 | 49.699 | 1.00 | 37.25 |
| ATOM | 5224 | SG | CYS | 2277 | 25.782 | 20.074 | 49.257 | 1.00 | 40.54 |
| ATOM | 5225 | C | CYS | 2277 | 27.393 | 22.446 | 47.819 | 1.00 | 36.33 |
| ATOM | 5226 | O | CYS | 2277 | 26.625 | 23.121 | 47.140 | 1.00 | 37.14 |
| ATOM | 5227 | N | LYS | 2278 | 28.398 | 21.758 | 47.290 | 1.00 | 35.33 |
| ATOM | 5228 | CA | LYS | 2278 | 28.672 | 21.768 | 45.859 | 1.00 | 33.89 |
| ATOM | 5229 | CB | LYS | 2278 | 30.184 | 21.895 | 45.606 | 1.00 | 34.33 |
| ATOM | 5230 | CG | LYS | 2278 | 30.777 | 23.219 | 46.041 | 1.00 | 34.51 |
| ATOM | 5231 | CD | LYS | 2278 | 30.057 | 24.356 | 45.368 | 1.00 | 36.11 |
| ATOM | 5232 | CE | LYS | 2278 | 30.501 | 25.693 | 45.935 | 1.00 | 38.57 |
| ATOM | 5233 | NZ | LYS | 2278 | 29.679 | 26.822 | 45.375 | 1.00 | 39.74 |
| ATOM | 5234 | C | LYS | 2278 | 28.148 | 20.496 | 45.233 | 1.00 | 32.29 |
| ATOM | 5235 | O | LYS | 2278 | 28.611 | 19.398 | 45.543 | 1.00 | 32.62 |
| ATOM | 5236 | N | VAL | 2279 | 27.199 | 20.654 | 44.325 | 1.00 | 30.07 |
| ATOM | 5237 | CA | VAL | 2279 | 26.566 | 19.523 | 43.691 | 1.00 | 28.30 |
| ATOM | 5238 | CB | VAL | 2279 | 25.026 | 19.633 | 43.917 | 1.00 | 27.83 |
| ATOM | 5239 | CG1 | VAL | 2279 | 24.255 | 18.558 | 43.136 | 1.00 | 25.87 |
| ATOM | 5240 | CG2 | VAL | 2279 | 24.739 | 19.539 | 45.406 | 1.00 | 25.86 |
| ATOM | 5241 | C | VAL | 2279 | 26.875 | 19.379 | 42.214 | 1.00 | 27.95 |
| ATOM | 5242 | O | VAL | 2279 | 26.966 | 20.364 | 41.488 | 1.00 | 28.45 |
| ATOM | 5243 | N | TYR | 2280 | 27.070 | 18.141 | 41.780 | 1.00 | 26.99 |
| ATOM | 5244 | CA | TYR | 2280 | 27.303 | 17.878 | 40.379 | 1.00 | 27.05 |
| ATOM | 5245 | CB | TYR | 2280 | 28.702 | 17.347 | 40.124 | 1.00 | 27.36 |
| ATOM | 5246 | CG | TYR | 2280 | 28.831 | 16.935 | 38.692 | 1.00 | 28.28 |
| ATOM | 5247 | CD1 | TYR | 2280 | 28.909 | 17.887 | 37.677 | 1.00 | 28.68 |
| ATOM | 5248 | CE1 | TYR | 2280 | 28.846 | 17.512 | 36.344 | 1.00 | 29.45 |
| ATOM | 5249 | CD2 | TYR | 2280 | 28.709 | 15.597 | 38.331 | 1.00 | 28.79 |
| ATOM | 5250 | CE2 | TYR | 2280 | 28.642 | 15.213 | 37.010 | 1.00 | 29.34 |
| ATOM | 5251 | CZ | TYR | 2280 | 28.705 | 16.165 | 36.021 | 1.00 | 30.30 |
| ATOM | 5252 | OH | TYR | 2280 | 28.579 | 15.758 | 34.711 | 1.00 | 32.24 |
| ATOM | 5253 | C | TYR | 2280 | 26.268 | 16.849 | 39.919 | 1.00 | 27.25 |
| ATOM | 5254 | O | TYR | 2280 | 26.029 | 15.843 | 40.592 | 1.00 | 27.12 |
| ATOM | 5255 | N | SER | 2281 | 25.639 | 17.114 | 38.778 | 1.00 | 26.82 |
| ATOM | 5256 | CA | SER | 2281 | 24.625 | 16.219 | 38.261 | 1.00 | 26.86 |
| ATOM | 5257 | CB | SER | 2281 | 23.366 | 16.348 | 39.115 | 1.00 | 26.81 |
| ATOM | 5258 | OG | SER | 2281 | 22.408 | 15.362 | 38.777 | 1.00 | 27.31 |
| ATOM | 5259 | C | SER | 2281 | 24.306 | 16.582 | 36.821 | 1.00 | 27.08 |
| ATOM | 5260 | O | SER | 2281 | 24.061 | 17.747 | 36.527 | 1.00 | 27.38 |
| ATOM | 5261 | N | ASP | 2282 | 24.329 | 15.598 | 35.921 | 1.00 | 27.14 |
| ATOM | 5262 | CA | ASP | 2282 | 24.006 | 15.853 | 34.521 | 1.00 | 27.01 |
| ATOM | 5263 | CB | ASP | 2282 | 24.371 | 14.643 | 33.659 | 1.00 | 26.51 |
| ATOM | 5264 | CG | ASP | 2282 | 24.096 | 14.861 | 32.184 | 1.00 | 26.84 |
| ATOM | 5265 | OD1 | ASP | 2282 | 24.658 | 14.087 | 31.374 | 1.00 | 26.84 |
| ATOM | 5266 | OD2 | ASP | 2282 | 23.326 | 15.780 | 31.829 | 1.00 | 26.00 |
| ATOM | 5267 | C | ASP | 2282 | 22.497 | 16.131 | 34.505 | 1.00 | 27.80 |
| ATOM | 5268 | O | ASP | 2282 | 22.055 | 17.230 | 34.149 | 1.00 | 26.80 |
| ATOM | 5269 | N | PRO | 2283 | 21.680 | 15.135 | 34.885 | 1.00 | 28.45 |
| ATOM | 5270 | CD | PRO | 2283 | 21.932 | 13.737 | 35.289 | 1.00 | 27.92 |
| ATOM | 5271 | CA | PRO | 2283 | 20.244 | 15.453 | 34.875 | 1.00 | 28.70 |
| ATOM | 5272 | CB | PRO | 2283 | 19.578 | 14.132 | 35.294 | 1.00 | 28.65 |
| ATOM | 5273 | CG | PRO | 2283 | 20.618 | 13.067 | 34.966 | 1.00 | 29.28 |
| ATOM | 5274 | C | PRO | 2283 | 20.058 | 16.530 | 35.956 | 1.00 | 28.97 |
| ATOM | 5275 | O | PRO | 2283 | 20.792 | 16.561 | 36.950 | 1.00 | 29.10 |
| ATOM | 5276 | N | GLN | 2284 | 19.083 | 17.406 | 35.766 | 1.00 | 28.73 |
| ATOM | 5277 | CA | GLN | 2284 | 18.802 | 18.470 | 36.719 | 1.00 | 28.58 |
| ATOM | 5278 | CB | GLN | 2284 | 17.558 | 19.216 | 36.235 | 1.00 | 27.98 |
| ATOM | 5279 | CG | GLN | 2284 | 17.659 | 20.711 | 36.280 | 1.00 | 28.35 |
| ATOM | 5280 | CD | GLN | 2284 | 18.956 | 21.225 | 35.701 | 1.00 | 27.84 |
| ATOM | 5281 | OE1 | GLN | 2284 | 19.246 | 21.049 | 34.517 | 1.00 | 27.07 |
| ATOM | 5282 | NE2 | GLN | 2284 | 19.748 | 21.867 | 36.543 | 1.00 | 28.56 |
| ATOM | 5283 | C | GLN | 2284 | 18.577 | 17.875 | 38.125 | 1.00 | 28.66 |
| ATOM | 5284 | O | GLN | 2284 | 17.706 | 17.022 | 38.311 | 1.00 | 29.74 |
| ATOM | 5285 | N | PRO | 2285 | 19.367 | 18.300 | 39.125 | 1.00 | 27.63 |
| ATOM | 5286 | CD | PRO | 2285 | 20.679 | 18.973 | 39.023 | 1.00 | 26.94 |
| ATOM | 5287 | CA | PRO | 2285 | 19.181 | 17.755 | 40.471 | 1.00 | 27.19 |
| ATOM | 5288 | CB | PRO | 2285 | 20.601 | 17.724 | 41.028 | 1.00 | 26.31 |
| ATOM | 5289 | CG | PRO | 2285 | 21.168 | 18.985 | 40.481 | 1.00 | 25.33 |
| ATOM | 5290 | C | PRO | 2285 | 18.267 | 18.594 | 41.346 | 1.00 | 27.28 |
| ATOM | 5291 | O | PRO | 2285 | 18.134 | 19.800 | 41.149 | 1.00 | 26.60 |
| ATOM | 5292 | N | HIS | 2286 | 17.640 | 17.942 | 42.318 | 1.00 | 27.47 |
| ATOM | 5293 | CA | HIS | 2286 | 16.781 | 18.650 | 43.247 | 1.00 | 27.94 |
| ATOM | 5294 | CB | HIS | 2286 | 15.400 | 18.001 | 43.363 | 1.00 | 26.81 |
| ATOM | 5295 | CG | HIS | 2286 | 14.482 | 18.746 | 44.277 | 1.00 | 26.92 |
| ATOM | 5296 | CD2 | HIS | 2286 | 13.988 | 20.006 | 44.206 | 1.00 | 27.50 |
| ATOM | 5297 | ND1 | HIS | 2286 | 14.074 | 18.246 | 45.494 | 1.00 | 26.29 |
| ATOM | 5298 | CE1 | HIS | 2286 | 13.375 | 19.165 | 46.136 | 1.00 | 26.25 |
| ATOM | 5299 | NE2 | HIS | 2286 | 13.307 | 20.243 | 45.376 | 1.00 | 26.93 |
| ATOM | 5300 | C | HIS | 2286 | 17.458 | 18.645 | 44.604 | 1.00 | 28.13 |
| ATOM | 5301 | O | HIS | 2286 | 17.597 | 17.592 | 45.221 | 1.00 | 28.27 |
| ATOM | 5302 | N | ILE | 2287 | 17.891 | 19.820 | 45.055 | 1.00 | 28.66 |
| ATOM | 5303 | CA | ILE | 2287 | 18.570 | 19.953 | 46.337 | 1.00 | 28.85 |
| ATOM | 5304 | CB | ILE | 2287 | 19.717 | 20.973 | 46.257 | 1.00 | 28.70 |
| ATOM | 5305 | CG2 | ILE | 2287 | 20.367 | 21.162 | 47.637 | 1.00 | 27.05 |
| ATOM | 5306 | CG1 | ILE | 2287 | 20.746 | 20.496 | 45.227 | 1.00 | 27.40 |
| ATOM | 5307 | CD1 | ILE | 2287 | 21.948 | 21.396 | 45.118 | 1.00 | 27.72 |
| ATOM | 5308 | C | ILE | 2287 | 17.618 | 20.347 | 47.454 | 1.00 | 29.85 |
| ATOM | 5309 | O | ILE | 2287 | 16.579 | 20.957 | 47.229 | 1.00 | 30.09 |
| ATOM | 5310 | N | GLN | 2288 | 17.995 | 19.999 | 48.673 | 1.00 | 30.86 |
| ATOM | 5311 | CA | GLN | 2288 | 17.160 | 20.269 | 49.822 | 1.00 | 31.69 |
| ATOM | 5312 | CB | GLN | 2288 | 16.149 | 19.130 | 49.908 | 1.00 | 32.71 |
| ATOM | 5313 | CG | GLN | 2288 | 15.134 | 19.188 | 51.006 | 1.00 | 34.82 |
| ATOM | 5314 | CD | GLN | 2288 | 14.112 | 18.062 | 50.873 | 1.00 | 35.54 |
| ATOM | 5315 | OE1 | GLN | 2288 | 13.117 | 18.021 | 51.597 | 1.00 | 36.68 |
| ATOM | 5316 | NE2 | GLN | 2288 | 14.359 | 17.141 | 49.939 | 1.00 | 35.12 |
| ATOM | 5317 | C | GLN | 2288 | 18.053 | 20.312 | 51.055 | 1.00 | 31.15 |
| ATOM | 5318 | O | GLN | 2288 | 18.982 | 19.521 | 51.160 | 1.00 | 31.72 |
| ATOM | 5319 | N | TRP | 2289 | 17.796 | 21.259 | 51.956 | 1.00 | 30.36 |
| ATOM | 5320 | CA | TRP | 2289 | 18.551 | 21.375 | 53.208 | 1.00 | 29.27 |
| ATOM | 5321 | CB | TRP | 2289 | 19.028 | 22.806 | 53.439 | 1.00 | 26.27 |
| ATOM | 5322 | CG | TRP | 2289 | 20.226 | 23.193 | 52.650 | 1.00 | 24.71 |
| ATOM | 5323 | CD2 | TRP | 2289 | 21.598 | 22.964 | 53.004 | 1.00 | 23.16 |
| ATOM | 5324 | CE2 | TRP | 2289 | 22.395 | 23.536 | 51.979 | 1.00 | 23.19 |
| ATOM | 5325 | CE3 | TRP | 2289 | 22.231 | 22.334 | 54.087 | 1.00 | 22.47 |
| ATOM | 5326 | CD1 | TRP | 2289 | 20.244 | 23.862 | 51.460 | 1.00 | 24.38 |
| ATOM | 5327 | NE1 | TRP | 2289 | 21.544 | 24.074 | 51.052 | 1.00 | 23.34 |
| ATOM | 5328 | CZ2 | TRP | 2289 | 23.801 | 23.502 | 52.004 | 1.00 | 22.31 |
| ATOM | 5329 | CZ3 | TRP | 2289 | 23.636 | 22.294 | 54.115 | 1.00 | 23.80 |
| ATOM | 5330 | CH2 | TRP | 2289 | 24.402 | 22.880 | 53.073 | 1.00 | 23.16 |
| ATOM | 5331 | C | TRP | 2289 | 17.678 | 20.950 | 54.395 | 1.00 | 30.34 |
| ATOM | 5332 | O | TRP | 2289 | 16.557 | 21.441 | 54.568 | 1.00 | 29.75 |
| ATOM | 5333 | N | LEU | 2290 | 18.198 | 20.041 | 55.211 | 1.00 | 31.29 |
| ATOM | 5334 | CA | LEU | 2290 | 17.470 | 19.530 | 56.371 | 1.00 | 32.32 |
| ATOM | 5335 | CB | LEU | 2290 | 17.219 | 18.027 | 56.209 | 1.00 | 32.06 |
| ATOM | 5336 | CG | LEU | 2290 | 16.089 | 17.485 | 55.326 | 1.00 | 31.82 |
| ATOM | 5337 | CD1 | LEU | 2290 | 15.577 | 18.558 | 54.388 | 1.00 | 33.18 |
| ATOM | 5338 | CD2 | LEU | 2290 | 16.594 | 16.284 | 54.555 | 1.00 | 30.25 |
| ATOM | 5339 | C | LEU | 2290 | 18.215 | 19.740 | 57.671 | 1.00 | 32.98 |
| ATOM | 5340 | O | LEU | 2290 | 19.440 | 19.793 | 57.692 | 1.00 | 33.80 |
| ATOM | 5341 | N | LYS | 2291 | 17.460 | 19.860 | 58.754 | 1.00 | 33.86 |
| ATOM | 5342 | CA | LYS | 2291 | 18.025 | 20.007 | 60.090 | 1.00 | 34.84 |
| ATOM | 5343 | CB | LYS | 2291 | 17.568 | 21.313 | 60.747 | 1.00 | 34.69 |
| ATOM | 5344 | CG | LYS | 2291 | 17.641 | 21.356 | 62.281 | 1.00 | 33.43 |
| ATOM | 5345 | CD | LYS | 2291 | 19.062 | 21.408 | 62.814 | 1.00 | 33.94 |
| ATOM | 5346 | CE | LYS | 2291 | 19.122 | 22.101 | 64.189 | 1.00 | 34.55 |
| ATOM | 5347 | NZ | LYS | 2291 | 18.273 | 21.470 | 65.260 | 1.00 | 34.70 |
| ATOM | 5348 | C | LYS | 2291 | 17.482 | 18.833 | 60.878 | 1.00 | 35.62 |
| ATOM | 5349 | O | LYS | 2291 | 16.274 | 18.614 | 60.911 | 1.00 | 35.11 |
| ATOM | 5350 | N | HIS | 2292 | 18.373 | 18.061 | 61.486 | 1.00 | 37.06 |
| ATOM | 5351 | CA | HIS | 2292 | 17.963 | 16.915 | 62.286 | 1.00 | 38.15 |
| ATOM | 5352 | CB | HIS | 2292 | 19.156 | 16.004 | 62.508 | 1.00 | 37.99 |
| ATOM | 5353 | CG | HIS | 2292 | 19.599 | 15.310 | 61.264 | 1.00 | 39.30 |
| ATOM | 5354 | CD2 | HIS | 2292 | 20.374 | 15.735 | 60.236 | 1.00 | 40.38 |
| ATOM | 5355 | ND1 | HIS | 2292 | 19.134 | 14.065 | 60.905 | 1.00 | 39.35 |
| ATOM | 5356 | CE1 | HIS | 2292 | 19.598 | 13.755 | 59.708 | 1.00 | 39.86 |
| ATOM | 5357 | NE2 | HIS | 2292 | 20.352 | 14.752 | 59.279 | 1.00 | 39.95 |
| ATOM | 5358 | C | HIS | 2292 | 17.422 | 17.414 | 63.610 | 1.00 | 39.16 |
| ATOM | 5359 | O | HIS | 2292 | 18.103 | 18.140 | 64.331 | 1.00 | 39.28 |
| ATOM | 5360 | N | ILE | 2293 | 16.191 | 17.030 | 63.921 | 1.00 | 39.79 |
| ATOM | 5361 | CA | ILE | 2293 | 15.558 | 17.473 | 65.154 | 1.00 | 40.75 |
| ATOM | 5362 | CB | ILE | 2293 | 14.362 | 18.390 | 64.851 | 1.00 | 39.23 |
| ATOM | 5363 | CG2 | ILE | 2293 | 14.808 | 19.535 | 63.969 | 1.00 | 39.31 |
| ATOM | 5364 | CG1 | ILE | 2293 | 13.258 | 17.598 | 64.153 | 1.00 | 39.02 |
| ATOM | 5365 | CD1 | ILE | 2293 | 12.020 | 18.404 | 63.827 | 1.00 | 36.97 |
| ATOM | 5366 | C | ILE | 2293 | 15.060 | 16.344 | 66.041 | 1.00 | 42.01 |
| ATOM | 5367 | O | ILE | 2293 | 15.025 | 15.171 | 65.649 | 1.00 | 42.38 |
| ATOM | 5368 | N | GLU | 2294 | 14.677 | 16.722 | 67.253 | 1.00 | 43.78 |
| ATOM | 5369 | CA | GLU | 2294 | 14.137 | 15.780 | 68.219 | 1.00 | 45.28 |
| ATOM | 5370 | CB | GLU | 2294 | 14.842 | 15.919 | 69.565 | 1.00 | 45.66 |
| ATOM | 5371 | CG | GLU | 2294 | 16.138 | 15.152 | 69.686 | 1.00 | 48.34 |
| ATOM | 5372 | CD | GLU | 2294 | 16.717 | 15.220 | 71.090 | 1.00 | 50.36 |
| ATOM | 5373 | OE1 | GLU | 2294 | 15.939 | 15.075 | 72.059 | 1.00 | 50.33 |
| ATOM | 5374 | OE2 | GLU | 2294 | 17.947 | 15.407 | 71.230 | 1.00 | 51.59 |
| ATOM | 5375 | C | GLU | 2294 | 12.664 | 16.089 | 68.407 | 1.00 | 45.75 |
| ATOM | 5376 | O | GLU | 2294 | 12.295 | 17.241 | 68.611 | 1.00 | 45.16 |
| ATOM | 5377 | N | VAL | 2295 | 11.818 | 15.076 | 68.299 | 1.00 | 46.82 |
| ATOM | 5378 | CA | VAL | 2295 | 10.401 | 15.297 | 68.531 | 1.00 | 48.64 |
| ATOM | 5379 | CB | VAL | 2295 | 9.523 | 14.722 | 67.429 | 1.00 | 48.60 |
| ATOM | 5380 | CG1 | VAL | 2295 | 8.068 | 14.895 | 67.809 | 1.00 | 48.18 |
| ATOM | 5381 | CG2 | VAL | 2295 | 9.797 | 15.456 | 66.123 | 1.00 | 49.01 |
| ATOM | 5382 | C | VAL | 2295 | 10.155 | 14.569 | 69.829 | 1.00 | 50.22 |
| ATOM | 5383 | O | VAL | 2295 | 10.173 | 13.333 | 69.883 | 1.00 | 50.70 |
| ATOM | 5384 | N | ASN | 2296 | 9.950 | 15.364 | 70.875 | 1.00 | 51.38 |
| ATOM | 5385 | CA | ASN | 2296 | 9.771 | 14.867 | 72.225 | 1.00 | 52.31 |
| ATOM | 5386 | CB | ASN | 2296 | 8.815 | 13.678 | 72.253 | 1.00 | 52.69 |
| ATOM | 5387 | CG | ASN | 2296 | 7.385 | 14.081 | 71.915 | 1.00 | 54.24 |
| ATOM | 5388 | OD1 | ASN | 2296 | 6.840 | 15.032 | 72.497 | 1.00 | 54.33 |
| ATOM | 5389 | ND2 | ASN | 2296 | 6.767 | 13.358 | 70.980 | 1.00 | 53.98 |
| ATOM | 5390 | C | ASN | 2296 | 11.162 | 14.466 | 72.695 | 1.00 | 52.86 |
| ATOM | 5391 | O | ASN | 2296 | 12.020 | 15.328 | 72.918 | 1.00 | 53.10 |
| ATOM | 5392 | N | GLY | 2297 | 11.410 | 13.172 | 72.814 | 1.00 | 53.10 |
| ATOM | 5393 | CA | GLY | 2297 | 12.723 | 12.764 | 73.262 | 1.00 | 53.81 |
| ATOM | 5394 | C | GLY | 2297 | 13.551 | 12.059 | 72.213 | 1.00 | 54.12 |
| ATOM | 5395 | O | GLY | 2297 | 14.770 | 11.978 | 72.337 | 1.00 | 54.71 |
| ATOM | 5396 | N | SER | 2298 | 12.902 | 11.558 | 71.172 | 1.00 | 54.20 |
| ATOM | 5397 | CA | SER | 2298 | 13.616 | 10.825 | 70.144 | 1.00 | 54.33 |
| ATOM | 5398 | CB | SER | 2298 | 12.803 | 9.590 | 69.761 | 1.00 | 55.05 |
| ATOM | 5399 | OG | SER | 2298 | 11.420 | 9.853 | 69.911 | 1.00 | 55.50 |
| ATOM | 5400 | C | SER | 2298 | 14.022 | 11.588 | 68.893 | 1.00 | 54.45 |
| ATOM | 5401 | O | SER | 2298 | 13.343 | 12.514 | 68.437 | 1.00 | 54.45 |
| ATOM | 5402 | N | LYS | 2299 | 15.157 | 11.167 | 68.354 | 1.00 | 54.68 |
| ATOM | 5403 | CA | LYS | 2299 | 15.724 | 11.742 | 67.151 | 1.00 | 55.54 |
| ATOM | 5404 | CB | LYS | 2299 | 17.246 | 11.739 | 67.245 | 1.00 | 56.00 |
| ATOM | 5405 | CG | LYS | 2299 | 17.778 | 12.472 | 68.444 | 1.00 | 57.41 |
| ATOM | 5406 | CD | LYS | 2299 | 19.267 | 12.715 | 68.318 | 1.00 | 58.81 |
| ATOM | 5407 | CE | LYS | 2299 | 19.745 | 13.695 | 69.385 | 1.00 | 59.30 |
| ATOM | 5408 | NZ | LYS | 2299 | 21.150 | 14.124 | 69.125 | 1.00 | 59.81 |
| ATOM | 5409 | C | LYS | 2299 | 15.300 | 10.898 | 65.961 | 1.00 | 55.78 |
| ATOM | 5410 | O | LYS | 2299 | 15.299 | 11.364 | 64.822 | 1.00 | 55.56 |
| ATOM | 5411 | N | ILE | 2300 | 14.946 | 9.649 | 66.239 | 1.00 | 56.11 |
| ATOM | 5412 | CA | ILE | 2300 | 14.535 | 8.720 | 65.197 | 1.00 | 56.72 |
| ATOM | 5413 | CB | ILE | 2300 | 15.381 | 7.423 | 65.244 | 1.00 | 56.91 |
| ATOM | 5414 | CG2 | ILE | 2300 | 15.100 | 6.577 | 64.014 | 1.00 | 57.24 |
| ATOM | 5415 | CG1 | ILE | 2300 | 16.878 | 7.769 | 65.310 | 1.00 | 57.13 |
| ATOM | 5416 | CD1 | ILE | 2300 | 17.375 | 8.658 | 64.181 | 1.00 | 56.77 |
| ATOM | 5417 | C | ILE | 2300 | 13.060 | 8.361 | 65.344 | 1.00 | 56.82 |
| ATOM | 5418 | O | ILE | 2300 | 12.669 | 7.656 | 66.274 | 1.00 | 56.94 |
| ATOM | 5419 | N | GLY | 2301 | 12.252 | 8.853 | 64.410 | 1.00 | 57.06 |
| ATOM | 5420 | CA | GLY | 2301 | 10.824 | 8.604 | 64.438 | 1.00 | 57.25 |
| ATOM | 5421 | C | GLY | 2301 | 10.414 | 7.142 | 64.370 | 1.00 | 57.57 |
| ATOM | 5422 | O | GLY | 2301 | 11.260 | 6.249 | 64.249 | 1.00 | 57.71 |
| ATOM | 5423 | N | PRO | 2302 | 9.098 | 6.875 | 64.430 | 1.00 | 57.35 |
| ATOM | 5424 | CD | PRO | 2302 | 8.060 | 7.924 | 64.382 | 1.00 | 57.34 |
| ATOM | 5425 | CA | PRO | 2302 | 8.485 | 5.540 | 64.386 | 1.00 | 57.08 |
| ATOM | 5426 | CB | PRO | 2302 | 6.993 | 5.854 | 64.216 | 1.00 | 57.20 |
| ATOM | 5427 | CG | PRO | 2302 | 6.842 | 7.187 | 64.880 | 1.00 | 57.20 |
| ATOM | 5428 | C | PRO | 2302 | 9.013 | 4.685 | 63.230 | 1.00 | 56.45 |
| ATOM | 5429 | O | PRO | 2302 | 9.571 | 3.597 | 63.428 | 1.00 | 56.39 |
| ATOM | 5430 | N | ASP | 2303 | 8.804 | 5.201 | 62.024 | 1.00 | 55.13 |
| ATOM | 5431 | CA | ASP | 2303 | 9.219 | 4.560 | 60.791 | 1.00 | 53.91 |
| ATOM | 5432 | CB | ASP | 2303 | 8.725 | 5.408 | 59.626 | 1.00 | 53.40 |
| ATOM | 5433 | CG | ASP | 2303 | 9.096 | 6.874 | 59.787 | 1.00 | 53.74 |
| ATOM | 5434 | OD1 | ASP | 2303 | 8.637 | 7.701 | 58.968 | 1.00 | 53.92 |
| ATOM | 5435 | OD2 | ASP | 2303 | 9.853 | 7.199 | 60.732 | 1.00 | 52.73 |
| ATOM | 5436 | C | ASP | 2303 | 10.739 | 4.391 | 60.716 | 1.00 | 53.37 |
| ATOM | 5437 | O | ASP | 2303 | 11.292 | 4.138 | 59.646 | 1.00 | 53.72 |
| ATOM | 5438 | N | ASN | 2304 | 11.410 | 4.528 | 61.853 | 1.00 | 52.21 |
| ATOM | 5439 | CA | ASN | 2304 | 12.861 | 4.404 | 61.916 | 1.00 | 51.05 |
| ATOM | 5440 | CB | ASN | 2304 | 13.303 | 2.998 | 61.482 | 1.00 | 51.38 |
| ATOM | 5441 | CG | ASN | 2304 | 14.780 | 2.721 | 61.781 | 1.00 | 51.23 |
| ATOM | 5442 | OD1 | ASN | 2304 | 15.253 | 2.959 | 62.895 | 1.00 | 50.48 |
| ATOM | 5443 | ND2 | ASN | 2304 | 15.507 | 2.204 | 60.788 | 1.00 | 50.71 |
| ATOM | 5444 | C | ASN | 2304 | 13.559 | 5.466 | 61.061 | 1.00 | 50.11 |
| ATOM | 5445 | O | ASN | 2304 | 14.775 | 5.408 | 60.851 | 1.00 | 50.01 |
| ATOM | 5446 | N | LEU | 2305 | 12.784 | 6.423 | 60.553 | 1.00 | 48.80 |
| ATOM | 5447 | CA | LEU | 2305 | 13.350 | 7.519 | 59.771 | 1.00 | 46.74 |
| ATOM | 5448 | CB | LEU | 2305 | 12.361 | 8.052 | 58.731 | 1.00 | 47.05 |
| ATOM | 5449 | CG | LEU | 2305 | 12.163 | 7.301 | 57.409 | 1.00 | 47.57 |
| ATOM | 5450 | CD1 | LEU | 2305 | 13.521 | 6.778 | 56.923 | 1.00 | 46.70 |
| ATOM | 5451 | CD2 | LEU | 2305 | 11.183 | 6.163 | 57.596 | 1.00 | 46.69 |
| ATOM | 5452 | C | LEU | 2305 | 13.643 | 8.619 | 60.774 | 1.00 | 45.44 |
| ATOM | 5453 | O | LEU | 2305 | 12.875 | 8.820 | 61.725 | 1.00 | 45.51 |
| ATOM | 5454 | N | PRO | 2306 | 14.760 | 9.339 | 60.594 | 1.00 | 43.52 |
| ATOM | 5455 | CD | PRO | 2306 | 15.829 | 9.134 | 59.602 | 1.00 | 42.73 |
| ATOM | 5456 | CA | PRO | 2306 | 15.103 | 10.421 | 61.525 | 1.00 | 42.28 |
| ATOM | 5457 | CB | PRO | 2306 | 16.563 | 10.701 | 61.199 | 1.00 | 42.21 |
| ATOM | 5458 | CG | PRO | 2306 | 16.624 | 10.414 | 59.719 | 1.00 | 42.70 |
| ATOM | 5459 | C | PRO | 2306 | 14.225 | 11.657 | 61.346 | 1.00 | 41.09 |
| ATOM | 5460 | O | PRO | 2306 | 13.897 | 12.032 | 60.228 | 1.00 | 40.46 |
| ATOM | 5461 | N | TYR | 2307 | 13.838 | 12.269 | 62.458 | 1.00 | 40.69 |
| ATOM | 5462 | CA | TYR | 2307 | 13.026 | 13.480 | 62.435 | 1.00 | 40.99 |
| ATOM | 5463 | CB | TYR | 2307 | 12.593 | 13.864 | 63.843 | 1.00 | 41.29 |
| ATOM | 5464 | CG | TYR | 2307 | 11.442 | 13.060 | 64.362 | 1.00 | 42.61 |
| ATOM | 5465 | CD1 | TYR | 2307 | 11.535 | 12.378 | 65.578 | 1.00 | 43.37 |
| ATOM | 5466 | CE1 | TYR | 2307 | 10.464 | 11.659 | 66.077 | 1.00 | 44.14 |
| ATOM | 5467 | CD2 | TYR | 2307 | 10.243 | 12.999 | 63.655 | 1.00 | 43.08 |
| ATOM | 5468 | CE2 | TYR | 2307 | 9.167 | 12.287 | 64.140 | 1.00 | 43.77 |
| ATOM | 5469 | CZ | TYR | 2307 | 9.280 | 11.615 | 65.351 | 1.00 | 44.75 |
| ATOM | 5470 | OH | TYR | 2307 | 8.212 | 10.880 | 65.826 | 1.00 | 45.77 |
| ATOM | 5471 | C | TYR | 2307 | 13.829 | 14.629 | 61.859 | 1.00 | 40.84 |
| ATOM | 5472 | O | TYR | 2307 | 14.848 | 15.028 | 62.425 | 1.00 | 41.19 |
| ATOM | 5473 | N | VAL | 2308 | 13.357 | 15.174 | 60.746 | 1.00 | 41.07 |
| ATOM | 5474 | CA | VAL | 2308 | 14.041 | 16.277 | 60.096 | 1.00 | 40.96 |
| ATOM | 5475 | CB | VAL | 2308 | 14.704 | 15.839 | 58.771 | 1.00 | 40.38 |
| ATOM | 5476 | CG1 | VAL | 2308 | 15.678 | 14.720 | 59.037 | 1.00 | 40.47 |
| ATOM | 5477 | CG2 | VAL | 2308 | 13.651 | 15.408 | 57.768 | 1.00 | 39.61 |
| ATOM | 5478 | C | VAL | 2308 | 13.118 | 17.446 | 59.797 | 1.00 | 40.88 |
| ATOM | 5479 | O | VAL | 2308 | 11.903 | 17.296 | 59.739 | 1.00 | 40.63 |
| ATOM | 5480 | N | GLN | 2309 | 13.717 | 18.618 | 59.619 | 1.00 | 40.52 |
| ATOM | 5481 | CA | GLN | 2309 | 12.971 | 19.825 | 59.299 | 1.00 | 39.92 |
| ATOM | 5482 | CB | GLN | 2309 | 13.166 | 20.899 | 60.365 | 1.00 | 40.28 |
| ATOM | 5483 | CG | GLN | 2309 | 12.279 | 22.122 | 60.128 | 1.00 | 41.08 |
| ATOM | 5484 | CD | GLN | 2309 | 12.658 | 23.323 | 60.980 | 1.00 | 41.87 |
| ATOM | 5485 | OE1 | GLN | 2309 | 13.201 | 23.182 | 62.075 | 1.00 | 42.00 |
| ATOM | 5486 | NE2 | GLN | 2309 | 12.355 | 24.518 | 60.481 | 1.00 | 42.72 |
| ATOM | 5487 | C | GLN | 2309 | 13.499 | 20.364 | 57.979 | 1.00 | 39.39 |
| ATOM | 5488 | O | GLN | 2309 | 14.695 | 20.628 | 57.861 | 1.00 | 39.16 |
| ATOM | 5489 | N | ILE | 2310 | 12.623 | 20.514 | 56.987 | 1.00 | 38.15 |
| ATOM | 5490 | CA | ILE | 2310 | 13.047 | 21.043 | 55.697 | 1.00 | 37.31 |
| ATOM | 5491 | CB | ILE | 2310 | 11.984 | 20.881 | 54.610 | 1.00 | 36.91 |
| ATOM | 5492 | CG2 | ILE | 2310 | 12.620 | 21.102 | 53.256 | 1.00 | 36.40 |
| ATOM | 5493 | CG1 | ILE | 2310 | 11.373 | 19.484 | 54.660 | 1.00 | 37.26 |
| ATOM | 5494 | CD1 | ILE | 2310 | 12.337 | 18.379 | 54.373 | 1.00 | 37.59 |
| ATOM | 5495 | C | ILE | 2310 | 13.290 | 22.530 | 55.876 | 1.00 | 36.95 |
| ATOM | 5496 | O | ILE | 2310 | 12.376 | 23.286 | 56.203 | 1.00 | 37.05 |
| ATOM | 5497 | N | LEU | 2311 | 14.524 | 22.956 | 55.650 | 1.00 | 36.63 |
| ATOM | 5498 | CA | LEU | 2311 | 14.875 | 24.358 | 55.825 | 1.00 | 35.47 |
| ATOM | 5499 | CB | LEU | 2311 | 16.253 | 24.493 | 56.473 | 1.00 | 33.99 |
| ATOM | 5500 | CG | LEU | 2311 | 16.503 | 23.808 | 57.814 | 1.00 | 33.23 |
| ATOM | 5501 | CD1 | LEU | 2311 | 17.915 | 24.103 | 58.300 | 1.00 | 32.38 |
| ATOM | 5502 | CD2 | LEU | 2311 | 15.482 | 24.302 | 58.810 | 1.00 | 33.95 |
| ATOM | 5503 | C | LEU | 2311 | 14.903 | 25.108 | 54.518 | 1.00 | 35.24 |
| ATOM | 5504 | O | LEU | 2311 | 14.749 | 26.323 | 54.494 | 1.00 | 35.26 |
| ATOM | 5505 | N | LYS | 2312 | 15.097 | 24.386 | 53.425 | 1.00 | 35.28 |
| ATOM | 5506 | CA | LYS | 2312 | 15.194 | 25.029 | 52.126 | 1.00 | 34.87 |
| ATOM | 5507 | CB | LYS | 2312 | 16.589 | 25.629 | 52.011 | 1.00 | 34.49 |
| ATOM | 5508 | CG | LYS | 2312 | 16.748 | 26.758 | 51.048 | 1.00 | 33.76 |
| ATOM | 5509 | CD | LYS | 2312 | 18.099 | 27.376 | 51.281 | 1.00 | 34.11 |
| ATOM | 5510 | CE | LYS | 2312 | 18.337 | 28.535 | 50.361 | 1.00 | 34.17 |
| ATOM | 5511 | NZ | LYS | 2312 | 19.651 | 29.133 | 50.670 | 1.00 | 34.82 |
| ATOM | 5512 | C | LYS | 2312 | 14.976 | 23.980 | 51.047 | 1.00 | 34.95 |
| ATOM | 5513 | O | LYS | 2312 | 15.557 | 22.899 | 51.106 | 1.00 | 35.05 |
| ATOM | 5514 | N | THR | 2313 | 14.132 | 24.291 | 50.073 | 1.00 | 35.00 |
| ATOM | 5515 | CA | THR | 2313 | 13.863 | 23.355 | 48.997 | 1.00 | 35.62 |
| ATOM | 5516 | CB | THR | 2313 | 12.530 | 22.648 | 49.193 | 1.00 | 35.29 |
| ATOM | 5517 | OG1 | THR | 2313 | 12.677 | 21.684 | 50.235 | 1.00 | 36.74 |
| ATOM | 5518 | CG2 | THR | 2313 | 12.102 | 21.935 | 47.916 | 1.00 | 35.40 |
| ATOM | 5519 | C | THR | 2313 | 13.868 | 24.002 | 47.627 | 1.00 | 36.00 |
| ATOM | 5520 | O | THR | 2313 | 13.198 | 25.009 | 47.394 | 1.00 | 36.81 |
| ATOM | 5521 | N | ALA | 2314 | 14.623 | 23.404 | 46.714 | 1.00 | 35.26 |
| ATOM | 5522 | CA | ALA | 2314 | 14.716 | 23.921 | 45.367 | 1.00 | 34.51 |
| ATOM | 5523 | CB | ALA | 2314 | 15.775 | 23.155 | 44.588 | 1.00 | 34.24 |
| ATOM | 5524 | C | ALA | 2314 | 13.368 | 23.829 | 44.665 | 1.00 | 33.90 |
| ATOM | 5525 | O | ALA | 2314 | 12.576 | 22.932 | 44.934 | 1.00 | 33.53 |
| ATOM | 5526 | N | GLY | 2315 | 13.124 | 24.774 | 43.764 | 1.00 | 33.85 |
| ATOM | 5527 | CA | GLY | 2315 | 11.889 | 24.814 | 43.013 | 1.00 | 34.12 |
| ATOM | 5528 | C | GLY | 2315 | 11.818 | 26.146 | 42.307 | 1.00 | 34.83 |
| ATOM | 5529 | O | GLY | 2315 | 12.781 | 26.913 | 42.328 | 1.00 | 34.57 |
| ATOM | 5530 | N | VAL | 2316 | 10.684 | 26.444 | 41.686 | 1.00 | 36.05 |
| ATOM | 5531 | CA | VAL | 2316 | 10.551 | 27.714 | 40.979 | 1.00 | 37.03 |
| ATOM | 5532 | CB | VAL | 2316 | 9.225 | 27.791 | 40.214 | 1.00 | 37.90 |
| ATOM | 5533 | CG1 | VAL | 2316 | 9.090 | 29.162 | 39.554 | 1.00 | 39.39 |
| ATOM | 5534 | CG2 | VAL | 2316 | 9.186 | 26.712 | 39.145 | 1.00 | 38.31 |
| ATOM | 5535 | C | VAL | 2316 | 10.662 | 28.934 | 41.895 | 1.00 | 37.04 |
| ATOM | 5536 | O | VAL | 2316 | 11.053 | 30.007 | 41.451 | 1.00 | 37.51 |
| ATOM | 5537 | N | ASN | 2317 | 10.340 | 28.766 | 43.172 | 1.00 | 37.09 |
| ATOM | 5538 | CA | ASN | 2317 | 10.406 | 29.864 | 44.136 | 1.00 | 37.40 |
| ATOM | 5539 | CB | ASN | 2317 | 9.372 | 29.645 | 45.246 | 1.00 | 37.28 |
| ATOM | 5540 | CG | ASN | 2317 | 7.931 | 29.766 | 44.750 | 1.00 | 37.21 |
| ATOM | 5541 | OD1 | ASN | 2317 | 7.059 | 28.989 | 45.149 | 1.00 | 36.37 |
| ATOM | 5542 | ND2 | ASN | 2317 | 7.675 | 30.749 | 43.893 | 1.00 | 36.59 |
| ATOM | 5543 | C | ASN | 2317 | 11.784 | 30.021 | 44.778 | 1.00 | 37.86 |
| ATOM | 5544 | O | ASN | 2317 | 12.117 | 31.085 | 45.304 | 1.00 | 38.28 |
| ATOM | 5545 | N | THR | 2318 | 12.574 | 28.952 | 44.755 | 1.00 | 37.94 |
| ATOM | 5546 | CA | THR | 2318 | 13.912 | 28.965 | 45.333 | 1.00 | 37.60 |
| ATOM | 5547 | CB | THR | 2318 | 13.997 | 28.096 | 46.601 | 1.00 | 37.26 |
| ATOM | 5548 | OG1 | THR | 2318 | 12.775 | 28.190 | 47.345 | 1.00 | 37.84 |
| ATOM | 5549 | CG2 | THR | 2318 | 15.142 | 28.548 | 47.467 | 1.00 | 36.41 |
| ATOM | 5550 | C | THR | 2318 | 14.821 | 28.336 | 44.293 | 1.00 | 38.14 |
| ATOM | 5551 | O | THR | 2318 | 15.080 | 27.137 | 44.333 | 1.00 | 37.61 |
| ATOM | 5552 | N | THR | 2319 | 15.287 | 29.153 | 43.357 | 1.00 | 38.66 |
| ATOM | 5553 | CA | THR | 2319 | 16.154 | 28.708 | 42.270 | 1.00 | 38.47 |
| ATOM | 5554 | CB | THR | 2319 | 16.420 | 29.884 | 41.314 | 1.00 | 38.22 |
| ATOM | 5555 | OG1 | THR | 2319 | 15.196 | 30.211 | 40.647 | 1.00 | 37.17 |
| ATOM | 5556 | CG2 | THR | 2319 | 17.503 | 29.540 | 40.282 | 1.00 | 39.24 |
| ATOM | 5557 | C | THR | 2319 | 17.481 | 28.133 | 42.740 | 1.00 | 38.48 |
| ATOM | 5558 | O | THR | 2319 | 17.913 | 28.369 | 43.873 | 1.00 | 37.69 |
| ATOM | 5559 | N | ASP | 2320 | 18.125 | 27.376 | 41.857 | 1.00 | 38.60 |
| ATOM | 5560 | CA | ASP | 2320 | 19.403 | 26.770 | 42.175 | 1.00 | 39.31 |
| ATOM | 5561 | CB | ASP | 2320 | 19.881 | 25.858 | 41.040 | 1.00 | 38.95 |
| ATOM | 5562 | CG | ASP | 2320 | 18.947 | 24.683 | 40.782 | 1.00 | 38.36 |
| ATOM | 5563 | OD1 | ASP | 2320 | 18.494 | 24.044 | 41.752 | 1.00 | 37.37 |
| ATOM | 5564 | OD2 | ASP | 2320 | 18.684 | 24.391 | 39.594 | 1.00 | 38.09 |
| ATOM | 5565 | C | ASP | 2320 | 20.486 | 27.800 | 42.462 | 1.00 | 40.12 |
| ATOM | 5566 | O | ASP | 2320 | 21.450 | 27.491 | 43.157 | 1.00 | 40.60 |
| ATOM | 5567 | N | LYS | 2321 | 20.337 | 29.018 | 41.941 | 1.00 | 40.90 |
| ATOM | 5568 | CA | LYS | 2321 | 21.353 | 30.042 | 42.155 | 1.00 | 41.80 |
| ATOM | 5569 | CB | LYS | 2321 | 20.914 | 31.387 | 41.569 | 1.00 | 43.44 |
| ATOM | 5570 | CG | LYS | 2321 | 19.810 | 32.064 | 42.342 | 1.00 | 47.12 |
| ATOM | 5571 | CD | LYS | 2321 | 19.446 | 33.423 | 41.759 | 1.00 | 48.65 |
| ATOM | 5572 | CE | LYS | 2321 | 18.340 | 34.057 | 42.600 | 1.00 | 50.98 |
| ATOM | 5573 | NZ | LYS | 2321 | 17.755 | 35.282 | 41.974 | 1.00 | 53.35 |
| ATOM | 5574 | C | LYS | 2321 | 21.672 | 30.190 | 43.636 | 1.00 | 41.56 |
| ATOM | 5575 | O | LYS | 2321 | 22.778 | 30.557 | 44.002 | 1.00 | 41.17 |
| ATOM | 5576 | N | GLU | 2322 | 20.717 | 29.850 | 44.488 | 1.00 | 41.77 |
| ATOM | 5577 | CA | GLU | 2322 | 20.910 | 29.986 | 45.920 | 1.00 | 42.24 |
| ATOM | 5578 | CB | GLU | 2322 | 19.905 | 30.997 | 46.437 | 1.00 | 43.76 |
| ATOM | 5579 | CG | GLU | 2322 | 18.503 | 30.623 | 46.045 | 1.00 | 46.69 |
| ATOM | 5580 | CD | GLU | 2322 | 17.515 | 31.730 | 46.309 | 1.00 | 48.52 |
| ATOM | 5581 | OE1 | GLU | 2322 | 17.261 | 32.034 | 47.503 | 1.00 | 49.41 |
| ATOM | 5582 | OE2 | GLU | 2322 | 17.000 | 32.296 | 45.314 | 1.00 | 49.57 |
| ATOM | 5583 | C | GLU | 2322 | 20.785 | 28.713 | 46.749 | 1.00 | 41.07 |
| ATOM | 5584 | O | GLU | 2322 | 20.782 | 28.777 | 47.973 | 1.00 | 41.26 |
| ATOM | 5585 | N | MSE | 2323 | 20.687 | 27.561 | 46.103 | 1.00 | 39.61 |
| ATOM | 5586 | CA | MSE | 2323 | 20.538 | 26.319 | 46.848 | 1.00 | 37.88 |
| ATOM | 5587 | CB | MSE | 2323 | 19.806 | 25.275 | 46.003 | 1.00 | 35.05 |
| ATOM | 5588 | CG | MSE | 2323 | 18.309 | 25.537 | 45.788 | 1.00 | 30.55 |
| ATOM | 5589 | SE | MSE | 2323 | 17.386 | 25.726 | 47.320 | 1.00 | 25.96 |
| ATOM | 5590 | CE | MSE | 2323 | 17.938 | 24.311 | 48.279 | 1.00 | 26.31 |
| ATOM | 5591 | C | MSE | 2323 | 21.835 | 25.722 | 47.365 | 1.00 | 38.12 |
| ATOM | 5592 | O | MSE | 2323 | 21.807 | 24.839 | 48.209 | 1.00 | 38.50 |
| ATOM | 5593 | N | GLU | 2324 | 22.976 | 26.195 | 46.882 | 1.00 | 38.57 |
| ATOM | 5594 | CA | GLU | 2324 | 24.230 | 25.636 | 47.352 | 1.00 | 38.94 |
| ATOM | 5595 | CB | GLU | 2324 | 25.285 | 25.683 | 46.253 | 1.00 | 39.03 |
| ATOM | 5596 | CG | GLU | 2324 | 25.017 | 24.692 | 45.124 | 1.00 | 39.58 |
| ATOM | 5597 | CD | GLU | 2324 | 26.230 | 24.489 | 44.216 | 1.00 | 40.09 |
| ATOM | 5598 | OE1 | GLU | 2324 | 26.813 | 25.503 | 43.770 | 1.00 | 40.77 |
| ATOM | 5599 | OE2 | GLU | 2324 | 26.597 | 23.322 | 43.943 | 1.00 | 39.43 |
| ATOM | 5600 | C | GLU | 2324 | 24.755 | 26.266 | 48.638 | 1.00 | 39.20 |
| ATOM | 5601 | O | GLU | 2324 | 25.880 | 26.015 | 49.046 | 1.00 | 39.79 |
| ATOM | 5602 | N | VAL | 2325 | 23.936 | 27.074 | 49.293 | 1.00 | 39.49 |
| ATOM | 5603 | CA | VAL | 2325 | 24.351 | 27.668 | 50.552 | 1.00 | 39.69 |
| ATOM | 5604 | CB | VAL | 2325 | 25.033 | 29.039 | 50.355 | 1.00 | 40.22 |
| ATOM | 5605 | CG1 | VAL | 2325 | 24.061 | 30.036 | 49.750 | 1.00 | 40.63 |
| ATOM | 5606 | CG2 | VAL | 2325 | 25.553 | 29.545 | 51.686 | 1.00 | 40.30 |
| ATOM | 5607 | C | VAL | 2325 | 23.163 | 27.825 | 51.487 | 1.00 | 39.79 |
| ATOM | 5608 | O | VAL | 2325 | 22.054 | 28.125 | 51.058 | 1.00 | 40.27 |
| ATOM | 5609 | N | LEU | 2326 | 23.395 | 27.583 | 52.770 | 1.00 | 39.40 |
| ATOM | 5610 | CA | LEU | 2326 | 22.342 | 27.716 | 53.769 | 1.00 | 39.20 |
| ATOM | 5611 | CB | LEU | 2326 | 22.081 | 26.374 | 54.467 | 1.00 | 37.59 |
| ATOM | 5612 | CG | LEU | 2326 | 21.116 | 26.375 | 55.666 | 1.00 | 35.15 |
| ATOM | 5613 | CD1 | LEU | 2326 | 19.740 | 26.806 | 55.225 | 1.00 | 34.39 |
| ATOM | 5614 | CD2 | LEU | 2326 | 21.063 | 25.000 | 56.287 | 1.00 | 32.79 |
| ATOM | 5615 | C | LEU | 2326 | 22.797 | 28.755 | 54.783 | 1.00 | 39.66 |
| ATOM | 5616 | O | LEU | 2326 | 23.851 | 28.600 | 55.396 | 1.00 | 39.64 |
| ATOM | 5617 | N | HIS | 2327 | 22.002 | 29.811 | 54.946 | 1.00 | 40.11 |
| ATOM | 5618 | CA | HIS | 2327 | 22.319 | 30.892 | 55.875 | 1.00 | 40.64 |
| ATOM | 5619 | CB | HIS | 2327 | 22.012 | 32.250 | 55.239 | 1.00 | 39.31 |
| ATOM | 5620 | CG | HIS | 2327 | 22.840 | 32.560 | 54.035 | 1.00 | 38.31 |
| ATOM | 5621 | CD2 | HIS | 2327 | 22.562 | 32.464 | 52.713 | 1.00 | 37.82 |
| ATOM | 5622 | ND1 | HIS | 2327 | 24.149 | 32.987 | 54.122 | 1.00 | 38.78 |
| ATOM | 5623 | CE1 | HIS | 2327 | 24.643 | 33.136 | 52.905 | 1.00 | 37.87 |
| ATOM | 5624 | NE2 | HIS | 2327 | 23.700 | 32.824 | 52.032 | 1.00 | 37.87 |
| ATOM | 5625 | C | HIS | 2327 | 21.544 | 30.791 | 57.183 | 1.00 | 41.82 |
| ATOM | 5626 | O | HIS | 2327 | 20.319 | 30.621 | 57.187 | 1.00 | 42.05 |
| ATOM | 5627 | N | LEU | 2328 | 22.267 | 30.898 | 58.291 | 1.00 | 42.60 |
| ATOM | 5628 | CA | LEU | 2328 | 21.661 | 30.861 | 59.612 | 1.00 | 43.72 |
| ATOM | 5629 | CB | LEU | 2328 | 22.167 | 29.657 | 60.402 | 1.00 | 42.78 |
| ATOM | 5630 | CG | LEU | 2328 | 21.869 | 28.275 | 59.817 | 1.00 | 42.23 |
| ATOM | 5631 | CD1 | LEU | 2328 | 22.379 | 27.213 | 60.770 | 1.00 | 40.90 |
| ATOM | 5632 | CD2 | LEU | 2328 | 20.380 | 28.111 | 59.590 | 1.00 | 40.44 |
| ATOM | 5633 | C | LEU | 2328 | 22.082 | 32.151 | 60.298 | 1.00 | 45.00 |
| ATOM | 5634 | O | LEU | 2328 | 23.271 | 32.422 | 60.448 | 1.00 | 45.19 |
| ATOM | 5635 | N | ARG | 2329 | 21.104 | 32.947 | 60.711 | 1.00 | 46.79 |
| ATOM | 5636 | CA | ARG | 2329 | 21.378 | 34.221 | 61.355 | 1.00 | 48.20 |
| ATOM | 5637 | CB | ARG | 2329 | 20.512 | 35.294 | 60.699 | 1.00 | 49.55 |
| ATOM | 5638 | CG | ARG | 2329 | 20.906 | 35.568 | 59.255 | 1.00 | 51.68 |
| ATOM | 5639 | CD | ARG | 2329 | 22.219 | 36.294 | 59.239 | 1.00 | 53.82 |
| ATOM | 5640 | NE | ARG | 2329 | 22.775 | 36.481 | 57.908 | 1.00 | 55.55 |
| ATOM | 5641 | CZ | ARG | 2329 | 23.752 | 37.339 | 57.629 | 1.00 | 56.78 |
| ATOM | 5642 | NH1 | ARG | 2329 | 24.277 | 38.091 | 58.585 | 1.00 | 55.71 |
| ATOM | 5643 | NH2 | ARG | 2329 | 24.207 | 37.441 | 56.384 | 1.00 | 58.30 |
| ATOM | 5644 | C | ARG | 2329 | 21.148 | 34.206 | 62.860 | 1.00 | 48.59 |
| ATOM | 5645 | O | ARG | 2329 | 20.212 | 33.574 | 63.344 | 1.00 | 48.74 |
| ATOM | 5646 | N | ASN | 2330 | 22.012 | 34.908 | 63.591 | 1.00 | 49.11 |
| ATOM | 5647 | CA | ASN | 2330 | 21.906 | 35.001 | 65.043 | 1.00 | 49.29 |
| ATOM | 5648 | CB | ASN | 2330 | 20.845 | 36.037 | 65.404 | 1.00 | 50.22 |
| ATOM | 5649 | CG | ASN | 2330 | 21.024 | 36.582 | 66.795 | 1.00 | 51.09 |
| ATOM | 5650 | OD1 | ASN | 2330 | 21.082 | 35.827 | 67.769 | 1.00 | 51.70 |
| ATOM | 5651 | ND2 | ASN | 2330 | 21.120 | 37.904 | 66.901 | 1.00 | 51.68 |
| ATOM | 5652 | C | ASN | 2330 | 21.517 | 33.645 | 65.612 | 1.00 | 49.00 |
| ATOM | 5653 | O | ASN | 2330 | 20.400 | 33.460 | 66.096 | 1.00 | 48.47 |
| ATOM | 5654 | N | VAL | 2331 | 22.445 | 32.698 | 65.545 | 1.00 | 49.18 |
| ATOM | 5655 | CA | VAL | 2331 | 22.191 | 31.343 | 66.019 | 1.00 | 49.30 |
| ATOM | 5656 | CB | VAL | 2331 | 23.330 | 30.384 | 65.611 | 1.00 | 48.73 |
| ATOM | 5657 | CG1 | VAL | 2331 | 23.430 | 30.315 | 64.103 | 1.00 | 47.86 |
| ATOM | 5658 | CG2 | VAL | 2331 | 24.648 | 30.845 | 66.215 | 1.00 | 48.06 |
| ATOM | 5659 | C | VAL | 2331 | 21.990 | 31.219 | 67.519 | 1.00 | 49.58 |
| ATOM | 5660 | O | VAL | 2331 | 22.659 | 31.887 | 68.304 | 1.00 | 49.20 |
| ATOM | 5661 | N | SER | 2332 | 21.055 | 30.354 | 67.901 | 1.00 | 49.83 |
| ATOM | 5662 | CA | SER | 2332 | 20.760 | 30.104 | 69.303 | 1.00 | 50.71 |
| ATOM | 5663 | CB | SER | 2332 | 19.253 | 30.113 | 69.534 | 1.00 | 51.34 |
| ATOM | 5664 | OG | SER | 2332 | 18.674 | 28.918 | 69.038 | 1.00 | 52.27 |
| ATOM | 5665 | C | SER | 2332 | 21.306 | 28.718 | 69.623 | 1.00 | 51.04 |
| ATOM | 5666 | O | SER | 2332 | 21.916 | 28.083 | 68.771 | 1.00 | 51.27 |
| ATOM | 5667 | N | PHE | 2333 | 21.090 | 28.250 | 70.848 | 1.00 | 51.51 |
| ATOM | 5668 | CA | PHE | 2333 | 21.552 | 26.922 | 71.232 | 1.00 | 51.88 |
| ATOM | 5669 | CB | PHE | 2333 | 21.506 | 26.762 | 72.755 | 1.00 | 52.11 |
| ATOM | 5670 | CG | PHE | 2333 | 22.704 | 27.349 | 73.455 | 1.00 | 52.73 |
| ATOM | 5671 | CD1 | PHE | 2333 | 22.565 | 28.020 | 74.665 | 1.00 | 53.22 |
| ATOM | 5672 | CD2 | PHE | 2333 | 23.983 | 27.224 | 72.902 | 1.00 | 52.51 |
| ATOM | 5673 | CE1 | PHE | 2333 | 23.683 | 28.558 | 75.315 | 1.00 | 53.24 |
| ATOM | 5674 | CE2 | PHE | 2333 | 25.098 | 27.755 | 73.540 | 1.00 | 52.79 |
| ATOM | 5675 | CZ | PHE | 2333 | 24.950 | 28.423 | 74.748 | 1.00 | 53.08 |
| ATOM | 5676 | C | PHE | 2333 | 20.670 | 25.888 | 70.562 | 1.00 | 51.59 |
| ATOM | 5677 | O | PHE | 2333 | 21.067 | 24.740 | 70.377 | 1.00 | 51.35 |
| ATOM | 5678 | N | GLU | 2334 | 19.469 | 26.315 | 70.187 | 1.00 | 51.94 |
| ATOM | 5679 | CA | GLU | 2334 | 18.526 | 25.428 | 69.532 | 1.00 | 52.27 |
| ATOM | 5680 | CB | GLU | 2334 | 17.123 | 26.048 | 69.501 | 1.00 | 54.00 |
| ATOM | 5681 | CG | GLU | 2334 | 16.434 | 26.182 | 70.863 | 1.00 | 56.62 |
| ATOM | 5682 | CD | GLU | 2334 | 16.839 | 27.444 | 71.627 | 1.00 | 58.50 |
| ATOM | 5683 | OE1 | GLU | 2334 | 17.974 | 27.499 | 72.155 | 1.00 | 59.24 |
| ATOM | 5684 | OE2 | GLU | 2334 | 16.016 | 28.390 | 71.692 | 1.00 | 59.46 |
| ATOM | 5685 | C | GLU | 2334 | 18.998 | 25.122 | 68.112 | 1.00 | 51.17 |
| ATOM | 5686 | O | GLU | 2334 | 18.755 | 24.032 | 67.593 | 1.00 | 51.65 |
| ATOM | 5687 | N | ASP | 2335 | 19.687 | 26.076 | 67.494 | 1.00 | 49.01 |
| ATOM | 5688 | CA | ASP | 2335 | 20.183 | 25.883 | 66.139 | 1.00 | 46.76 |
| ATOM | 5689 | CB | ASP | 2335 | 20.662 | 27.208 | 65.544 | 1.00 | 47.69 |
| ATOM | 5690 | CG | ASP | 2335 | 19.518 | 28.164 | 65.243 | 1.00 | 49.48 |
| ATOM | 5691 | OD1 | ASP | 2335 | 18.427 | 27.689 | 64.839 | 1.00 | 49.79 |
| ATOM | 5692 | OD2 | ASP | 2335 | 19.715 | 29.394 | 65.396 | 1.00 | 49.88 |
| ATOM | 5693 | C | ASP | 2335 | 21.303 | 24.854 | 66.038 | 1.00 | 44.87 |
| ATOM | 5694 | O | ASP | 2335 | 21.576 | 24.337 | 64.961 | 1.00 | 44.07 |
| ATOM | 5695 | N | ALA | 2336 | 21.960 | 24.555 | 67.151 | 1.00 | 42.98 |
| ATOM | 5696 | CA | ALA | 2336 | 23.036 | 23.575 | 67.134 | 1.00 | 40.92 |
| ATOM | 5697 | CB | ALA | 2336 | 23.644 | 23.455 | 68.495 | 1.00 | 40.33 |
| ATOM | 5698 | C | ALA | 2336 | 22.461 | 22.236 | 66.699 | 1.00 | 40.00 |
| ATOM | 5699 | O | ALA | 2336 | 21.272 | 21.979 | 66.888 | 1.00 | 39.77 |
| ATOM | 5700 | N | GLY | 2337 | 23.299 | 21.388 | 66.107 | 1.00 | 38.41 |
| ATOM | 5701 | CA | GLY | 2337 | 22.825 | 20.091 | 65.663 | 1.00 | 36.67 |
| ATOM | 5702 | C | GLY | 2337 | 23.347 | 19.662 | 64.304 | 1.00 | 36.23 |
| ATOM | 5703 | O | GLY | 2337 | 24.300 | 20.240 | 63.774 | 1.00 | 35.86 |
| ATOM | 5704 | N | GLU | 2338 | 22.712 | 18.647 | 63.726 | 1.00 | 35.12 |
| ATOM | 5705 | CA | GLU | 2338 | 23.136 | 18.129 | 62.439 | 1.00 | 34.42 |
| ATOM | 5706 | CB | GLU | 2338 | 23.084 | 16.601 | 62.460 | 1.00 | 33.62 |
| ATOM | 5707 | CG | GLU | 2338 | 23.609 | 15.953 | 61.192 | 1.00 | 35.00 |
| ATOM | 5708 | CD | GLU | 2338 | 23.736 | 14.443 | 61.313 | 1.00 | 36.05 |
| ATOM | 5709 | OE1 | GLU | 2338 | 22.781 | 13.813 | 61.828 | 1.00 | 36.23 |
| ATOM | 5710 | OE2 | GLU | 2338 | 24.782 | 13.888 | 60.886 | 1.00 | 35.37 |
| ATOM | 5711 | C | GLU | 2338 | 22.333 | 18.669 | 61.252 | 1.00 | 33.61 |
| ATOM | 5712 | O | GLU | 2338 | 21.103 | 18.632 | 61.246 | 1.00 | 33.58 |
| ATOM | 5713 | N | TYR | 2339 | 23.046 | 19.177 | 60.251 | 1.00 | 31.91 |
| ATOM | 5714 | CA | TYR | 2339 | 22.418 | 19.722 | 59.057 | 1.00 | 30.70 |
| ATOM | 5715 | CB | TYR | 2339 | 22.855 | 21.162 | 58.828 | 1.00 | 29.62 |
| ATOM | 5716 | CG | TYR | 2339 | 22.312 | 22.126 | 59.849 | 1.00 | 28.70 |
| ATOM | 5717 | CD1 | TYR | 2339 | 22.836 | 22.183 | 61.147 | 1.00 | 27.71 |
| ATOM | 5718 | CE1 | TYR | 2339 | 22.328 | 23.083 | 62.090 | 1.00 | 27.95 |
| ATOM | 5719 | CD2 | TYR | 2339 | 21.267 | 22.988 | 59.519 | 1.00 | 28.00 |
| ATOM | 5720 | CE2 | TYR | 2339 | 20.755 | 23.884 | 60.442 | 1.00 | 28.05 |
| ATOM | 5721 | CZ | TYR | 2339 | 21.282 | 23.931 | 61.720 | 1.00 | 28.10 |
| ATOM | 5722 | OH | TYR | 2339 | 20.739 | 24.819 | 62.612 | 1.00 | 27.66 |
| ATOM | 5723 | C | TYR | 2339 | 22.810 | 18.884 | 57.864 | 1.00 | 30.67 |
| ATOM | 5724 | O | TYR | 2339 | 23.968 | 18.476 | 57.728 | 1.00 | 31.09 |
| ATOM | 5725 | N | THR | 2340 | 21.850 | 18.634 | 56.986 | 1.00 | 29.93 |
| ATOM | 5726 | CA | THR | 2340 | 22.123 | 17.806 | 55.826 | 1.00 | 29.42 |
| ATOM | 5727 | CB | THR | 2340 | 21.381 | 16.436 | 55.936 | 1.00 | 28.47 |
| ATOM | 5728 | OG1 | THR | 2340 | 21.868 | 15.720 | 57.075 | 1.00 | 26.86 |
| ATOM | 5729 | CG2 | THR | 2340 | 21.595 | 15.599 | 54.685 | 1.00 | 26.96 |
| ATOM | 5730 | C | THR | 2340 | 21.760 | 18.448 | 54.492 | 1.00 | 29.66 |
| ATOM | 5731 | O | THR | 2340 | 20.753 | 19.149 | 54.368 | 1.00 | 28.27 |
| ATOM | 5732 | N | CYS | 2341 | 22.620 | 18.214 | 53.504 | 1.00 | 30.06 |
| ATOM | 5733 | CA | CYS | 2341 | 22.382 | 18.688 | 52.147 | 1.00 | 30.28 |
| ATOM | 5734 | CB | CYS | 2341 | 23.628 | 19.318 | 51.538 | 1.00 | 29.84 |
| ATOM | 5735 | SG | CYS | 2341 | 23.398 | 19.658 | 49.768 | 1.00 | 30.86 |
| ATOM | 5736 | C | CYS | 2341 | 22.003 | 17.434 | 51.351 | 1.00 | 30.59 |
| ATOM | 5737 | O | CYS | 2341 | 22.834 | 16.547 | 51.124 | 1.00 | 29.25 |
| ATOM | 5738 | N | LEU | 2342 | 20.741 | 17.370 | 50.948 | 1.00 | 30.22 |
| ATOM | 5739 | CA | LEU | 2342 | 20.212 | 16.237 | 50.222 | 1.00 | 29.91 |
| ATOM | 5740 | CB | LEU | 2342 | 18.877 | 15.872 | 50.860 | 1.00 | 29.44 |
| ATOM | 5741 | CG | LEU | 2342 | 18.071 | 14.679 | 50.360 | 1.00 | 29.94 |
| ATOM | 5742 | CD1 | LEU | 2342 | 18.885 | 13.404 | 50.486 | 1.00 | 29.69 |
| ATOM | 5743 | CD2 | LEU | 2342 | 16.785 | 14.588 | 51.181 | 1.00 | 28.73 |
| ATOM | 5744 | C | LEU | 2342 | 20.043 | 16.588 | 48.745 | 1.00 | 29.86 |
| ATOM | 5745 | O | LEU | 2342 | 19.448 | 17.604 | 48.409 | 1.00 | 30.73 |
| ATOM | 5746 | N | ALA | 2343 | 20.566 | 15.750 | 47.859 | 1.00 | 29.56 |
| ATOM | 5747 | CA | ALA | 2343 | 20.453 | 16.009 | 46.425 | 1.00 | 28.85 |
| ATOM | 5748 | CB | ALA | 2343 | 21.782 | 16.546 | 45.887 | 1.00 | 28.63 |
| ATOM | 5749 | C | ALA | 2343 | 20.062 | 14.748 | 45.673 | 1.00 | 27.76 |
| ATOM | 5750 | O | ALA | 2343 | 20.668 | 13.704 | 45.857 | 1.00 | 28.73 |
| ATOM | 5751 | N | GLY | 2344 | 19.053 | 14.836 | 44.823 | 1.00 | 27.01 |
| ATOM | 5752 | CA | GLY | 2344 | 18.666 | 13.657 | 44.079 | 1.00 | 26.44 |
| ATOM | 5753 | C | GLY | 2344 | 18.204 | 13.933 | 42.672 | 1.00 | 26.25 |
| ATOM | 5754 | O | GLY | 2344 | 17.879 | 15.063 | 42.329 | 1.00 | 25.47 |
| ATOM | 5755 | N | ASN | 2345 | 18.233 | 12.894 | 41.845 | 1.00 | 27.20 |
| ATOM | 5756 | CA | ASN | 2345 | 17.749 | 12.963 | 40.471 | 1.00 | 27.74 |
| ATOM | 5757 | CB | ASN | 2345 | 18.867 | 13.259 | 39.439 | 1.00 | 27.25 |
| ATOM | 5758 | CG | ASN | 2345 | 20.054 | 12.311 | 39.523 | 1.00 | 26.29 |
| ATOM | 5759 | OD1 | ASN | 2345 | 19.910 | 11.136 | 39.829 | 1.00 | 26.93 |
| ATOM | 5760 | ND2 | ASN | 2345 | 21.238 | 12.827 | 39.217 | 1.00 | 25.61 |
| ATOM | 5761 | C | ASN | 2345 | 17.071 | 11.623 | 40.209 | 1.00 | 27.96 |
| ATOM | 5762 | O | ASN | 2345 | 17.013 | 10.783 | 41.097 | 1.00 | 27.36 |
| ATOM | 5763 | N | SER | 2346 | 16.543 | 11.426 | 39.011 | 1.00 | 29.34 |
| ATOM | 5764 | CA | SER | 2346 | 15.850 | 10.185 | 38.701 | 1.00 | 30.55 |
| ATOM | 5765 | CB | SER | 2346 | 15.461 | 10.172 | 37.229 | 1.00 | 31.28 |
| ATOM | 5766 | OG | SER | 2346 | 16.402 | 10.922 | 36.468 | 1.00 | 35.03 |
| ATOM | 5767 | C | SER | 2346 | 16.656 | 8.943 | 39.038 | 1.00 | 30.81 |
| ATOM | 5768 | O | SER | 2346 | 16.096 | 7.928 | 39.460 | 1.00 | 31.47 |
| ATOM | 5769 | N | ILE | 2347 | 17.974 | 9.024 | 38.884 | 1.00 | 30.77 |
| ATOM | 5770 | CA | ILE | 2347 | 18.841 | 7.874 | 39.160 | 1.00 | 29.89 |
| ATOM | 5771 | CB | ILE | 2347 | 20.198 | 8.001 | 38.455 | 1.00 | 29.08 |
| ATOM | 5772 | CG2 | ILE | 2347 | 21.043 | 6.776 | 38.752 | 1.00 | 27.57 |
| ATOM | 5773 | CG1 | ILE | 2347 | 19.989 | 8.136 | 36.951 | 1.00 | 28.42 |
| ATOM | 5774 | CD1 | ILE | 2347 | 20.940 | 9.106 | 36.304 | 1.00 | 30.66 |
| ATOM | 5775 | C | ILE | 2347 | 19.122 | 7.559 | 40.623 | 1.00 | 30.07 |
| ATOM | 5776 | O | ILE | 2347 | 19.302 | 6.391 | 40.967 | 1.00 | 31.51 |
| ATOM | 5777 | N | GLY | 2348 | 19.175 | 8.573 | 41.485 | 1.00 | 29.89 |
| ATOM | 5778 | CA | GLY | 2348 | 19.450 | 8.306 | 42.888 | 1.00 | 29.38 |
| ATOM | 5779 | C | GLY | 2348 | 19.604 | 9.514 | 43.798 | 1.00 | 30.05 |
| ATOM | 5780 | O | GLY | 2348 | 19.551 | 10.663 | 43.355 | 1.00 | 30.31 |
| ATOM | 5781 | N | LEU | 2349 | 19.816 | 9.231 | 45.081 | 1.00 | 30.13 |
| ATOM | 5782 | CA | LEU | 2349 | 19.971 | 10.230 | 46.141 | 1.00 | 30.18 |
| ATOM | 5783 | CB | LEU | 2349 | 19.032 | 9.888 | 47.297 | 1.00 | 31.22 |
| ATOM | 5784 | CG | LEU | 2349 | 17.594 | 10.395 | 47.424 | 1.00 | 33.34 |
| ATOM | 5785 | CD1 | LEU | 2349 | 17.637 | 11.870 | 47.774 | 1.00 | 34.28 |
| ATOM | 5786 | CD2 | LEU | 2349 | 16.817 | 10.167 | 46.146 | 1.00 | 32.99 |
| ATOM | 5787 | C | LEU | 2349 | 21.388 | 10.293 | 46.729 | 1.00 | 30.22 |
| ATOM | 5788 | O | LEU | 2349 | 22.084 | 9.285 | 46.815 | 1.00 | 30.95 |
| ATOM | 5789 | N | SER | 2350 | 21.802 | 11.475 | 47.162 | 1.00 | 29.07 |
| ATOM | 5790 | CA | SER | 2350 | 23.108 | 11.649 | 47.785 | 1.00 | 28.61 |
| ATOM | 5791 | CB | SER | 2350 | 24.154 | 12.125 | 46.776 | 1.00 | 28.13 |
| ATOM | 5792 | OG | SER | 2350 | 24.477 | 11.116 | 45.849 | 1.00 | 28.81 |
| ATOM | 5793 | C | SER | 2350 | 22.968 | 12.692 | 48.878 | 1.00 | 28.39 |
| ATOM | 5794 | O | SER | 2350 | 22.117 | 13.580 | 48.794 | 1.00 | 27.93 |
| ATOM | 5795 | N | HIS | 2351 | 23.795 | 12.593 | 49.908 | 1.00 | 27.95 |
| ATOM | 5796 | CA | HIS | 2351 | 23.715 | 13.568 | 50.977 | 1.00 | 28.80 |
| ATOM | 5797 | CB | HIS | 2351 | 22.546 | 13.252 | 51.920 | 1.00 | 28.32 |
| ATOM | 5798 | CG | HIS | 2351 | 22.783 | 12.060 | 52.784 | 1.00 | 28.97 |
| ATOM | 5799 | CD2 | HIS | 2351 | 23.244 | 11.960 | 54.054 | 1.00 | 29.71 |
| ATOM | 5800 | ND1 | HIS | 2351 | 22.605 | 10.769 | 52.336 | 1.00 | 29.72 |
| ATOM | 5801 | CE1 | HIS | 2351 | 22.948 | 9.924 | 53.293 | 1.00 | 29.38 |
| ATOM | 5802 | NE2 | HIS | 2351 | 23.341 | 10.621 | 54.344 | 1.00 | 29.71 |
| ATOM | 5803 | C | HIS | 2351 | 24.997 | 13.648 | 51.783 | 1.00 | 28.90 |
| ATOM | 5804 | O | HIS | 2351 | 25.747 | 12.675 | 51.885 | 1.00 | 29.30 |
| ATOM | 5805 | N | HIS | 2352 | 25.237 | 14.827 | 52.345 | 1.00 | 28.59 |
| ATOM | 5806 | CA | HIS | 2352 | 26.400 | 15.096 | 53.179 | 1.00 | 28.39 |
| ATOM | 5807 | CB | HIS | 2352 | 27.389 | 16.038 | 52.476 | 1.00 | 28.83 |
| ATOM | 5808 | CG | HIS | 2352 | 28.368 | 15.346 | 51.575 | 1.00 | 30.62 |
| ATOM | 5809 | CD2 | HIS | 2352 | 28.536 | 14.036 | 51.267 | 1.00 | 31.12 |
| ATOM | 5810 | ND1 | HIS | 2352 | 29.327 | 16.031 | 50.856 | 1.00 | 31.74 |
| ATOM | 5811 | CE1 | HIS | 2352 | 30.040 | 15.174 | 50.143 | 1.00 | 31.60 |
| ATOM | 5812 | NE2 | HIS | 2352 | 29.580 | 13.957 | 50.374 | 1.00 | 31.34 |
| ATOM | 5813 | C | HIS | 2352 | 25.837 | 15.784 | 54.410 | 1.00 | 28.22 |
| ATOM | 5814 | O | HIS | 2352 | 24.852 | 16.522 | 54.326 | 1.00 | 28.05 |
| ATOM | 5815 | N | SER | 2353 | 26.457 | 15.544 | 55.553 | 1.00 | 28.04 |
| ATOM | 5816 | CA | SER | 2353 | 25.994 | 16.147 | 56.785 | 1.00 | 28.02 |
| ATOM | 5817 | CB | SER | 2353 | 25.378 | 15.087 | 57.681 | 1.00 | 26.72 |
| ATOM | 5818 | OG | SER | 2353 | 24.294 | 14.483 | 57.014 | 1.00 | 28.61 |
| ATOM | 5819 | C | SER | 2353 | 27.135 | 16.827 | 57.505 | 1.00 | 28.73 |
| ATOM | 5820 | O | SER | 2353 | 28.299 | 16.484 | 57.311 | 1.00 | 28.84 |
| ATOM | 5821 | N | ALA | 2354 | 26.793 | 17.804 | 58.329 | 1.00 | 29.20 |
| ATOM | 5822 | CA | ALA | 2354 | 27.788 | 18.534 | 59.085 | 1.00 | 29.78 |
| ATOM | 5823 | CB | ALA | 2354 | 28.169 | 19.824 | 58.359 | 1.00 | 29.62 |
| ATOM | 5824 | C | ALA | 2354 | 27.151 | 18.850 | 60.411 | 1.00 | 30.68 |
| ATOM | 5825 | O | ALA | 2354 | 25.929 | 18.889 | 60.528 | 1.00 | 29.77 |
| ATOM | 5826 | N | TRP | 2355 | 27.986 | 19.070 | 61.412 | 1.00 | 32.45 |
| ATOM | 5827 | CA | TRP | 2355 | 27.504 | 19.385 | 62.736 | 1.00 | 34.06 |
| ATOM | 5828 | CB | TRP | 2355 | 28.183 | 18.467 | 63.756 | 1.00 | 35.85 |
| ATOM | 5829 | CG | TRP | 2355 | 27.193 | 17.678 | 64.547 | 1.00 | 38.67 |
| ATOM | 5830 | CD2 | TRP | 2355 | 26.819 | 16.315 | 64.327 | 1.00 | 39.69 |
| ATOM | 5831 | CE2 | TRP | 2355 | 25.750 | 16.027 | 65.216 | 1.00 | 40.54 |
| ATOM | 5832 | CE3 | TRP | 2355 | 27.276 | 15.313 | 63.462 | 1.00 | 40.67 |
| ATOM | 5833 | CD1 | TRP | 2355 | 26.372 | 18.147 | 65.538 | 1.00 | 39.63 |
| ATOM | 5834 | NE1 | TRP | 2355 | 25.500 | 17.161 | 65.940 | 1.00 | 40.42 |
| ATOM | 5835 | CZ2 | TRP | 2355 | 25.130 | 14.771 | 65.263 | 1.00 | 40.53 |
| ATOM | 5836 | CZ3 | TRP | 2355 | 26.657 | 14.060 | 63.507 | 1.00 | 42.07 |
| ATOM | 5837 | CH2 | TRP | 2355 | 25.592 | 13.803 | 64.406 | 1.00 | 41.22 |
| ATOM | 5838 | C | TRP | 2355 | 27.779 | 20.842 | 63.065 | 1.00 | 34.15 |
| ATOM | 5839 | O | TRP | 2355 | 28.846 | 21.370 | 62.741 | 1.00 | 34.33 |
| ATOM | 5840 | N | LEU | 2356 | 26.810 | 21.493 | 63.694 | 1.00 | 34.24 |
| ATOM | 5841 | CA | LEU | 2356 | 26.972 | 22.885 | 64.081 | 1.00 | 34.60 |
| ATOM | 5842 | CB | LEU | 2356 | 25.792 | 23.718 | 63.566 | 1.00 | 34.30 |
| ATOM | 5843 | CG | LEU | 2356 | 25.771 | 25.264 | 63.601 | 1.00 | 34.52 |
| ATOM | 5844 | CD1 | LEU | 2356 | 25.012 | 25.733 | 64.808 | 1.00 | 33.94 |
| ATOM | 5845 | CD2 | LEU | 2356 | 27.177 | 25.849 | 63.573 | 1.00 | 33.95 |
| ATOM | 5846 | C | LEU | 2356 | 27.043 | 22.940 | 65.598 | 1.00 | 35.24 |
| ATOM | 5847 | O | LEU | 2356 | 26.116 | 22.509 | 66.284 | 1.00 | 35.36 |
| ATOM | 5848 | N | THR | 2357 | 28.160 | 23.432 | 66.123 | 1.00 | 35.71 |
| ATOM | 5849 | CA | THR | 2357 | 28.320 | 23.553 | 67.562 | 1.00 | 36.50 |
| ATOM | 5850 | CB | THR | 2357 | 29.633 | 22.950 | 68.038 | 1.00 | 36.80 |
| ATOM | 5851 | OG1 | THR | 2357 | 29.586 | 21.536 | 67.838 | 1.00 | 37.50 |
| ATOM | 5852 | CG2 | THR | 2357 | 29.850 | 23.240 | 69.515 | 1.00 | 36.17 |
| ATOM | 5853 | C | THR | 2357 | 28.252 | 25.014 | 67.970 | 1.00 | 37.46 |
| ATOM | 5854 | O | THR | 2357 | 28.972 | 25.865 | 67.451 | 1.00 | 37.57 |
| ATOM | 5855 | N | VAL | 2358 | 27.364 | 25.297 | 68.909 | 1.00 | 39.05 |
| ATOM | 5856 | CA | VAL | 2358 | 27.161 | 26.658 | 69.394 | 1.00 | 40.47 |
| ATOM | 5857 | CB | VAL | 2358 | 25.652 | 27.033 | 69.348 | 1.00 | 40.22 |
| ATOM | 5858 | CG1 | VAL | 2358 | 25.448 | 28.473 | 69.801 | 1.00 | 39.63 |
| ATOM | 5859 | CG2 | VAL | 2358 | 25.113 | 26.819 | 67.939 | 1.00 | 39.20 |
| ATOM | 5860 | C | VAL | 2358 | 27.659 | 26.848 | 70.826 | 1.00 | 41.06 |
| ATOM | 5861 | O | VAL | 2358 | 27.292 | 26.092 | 71.726 | 1.00 | 41.47 |
| ATOM | 5862 | N | LEU | 2359 | 28.490 | 27.862 | 71.033 | 1.00 | 41.72 |
| ATOM | 5863 | CA | LEU | 2359 | 29.008 | 28.153 | 72.361 | 1.00 | 42.23 |
| ATOM | 5864 | CB | LEU | 2359 | 30.530 | 28.045 | 72.353 | 1.00 | 40.82 |
| ATOM | 5865 | CG | LEU | 2359 | 31.015 | 26.712 | 71.777 | 1.00 | 40.56 |
| ATOM | 5866 | CD1 | LEU | 2359 | 32.528 | 26.682 | 71.755 | 1.00 | 39.75 |
| ATOM | 5867 | CD2 | LEU | 2359 | 30.467 | 25.558 | 72.598 | 1.00 | 39.96 |
| ATOM | 5868 | C | LEU | 2359 | 28.566 | 29.553 | 72.796 | 1.00 | 42.82 |
| ATOM | 5869 | O | LEU | 2359 | 28.662 | 29.846 | 74.011 | 1.00 | 44.64 |
| ATOM | 5870 | CB | MSE | 3149 | 8.217 | −10.346 | −17.866 | 1.00 | 71.78 |
| ATOM | 5871 | CG | MSE | 3149 | 9.085 | −11.589 | −17.649 | 1.00 | 74.35 |
| ATOM | 5872 | SE | MSE | 3149 | 8.445 | −13.188 | −18.291 | 1.00 | 77.77 |
| ATOM | 5873 | CE | MSE | 3149 | 9.236 | −13.242 | −19.932 | 1.00 | 76.24 |
| ATOM | 5874 | C | MSE | 3149 | 7.115 | −10.623 | −15.631 | 1.00 | 70.14 |
| ATOM | 5875 | O | MSE | 3149 | 8.134 | −10.218 | −15.073 | 1.00 | 70.36 |
| ATOM | 5876 | N | MSE | 3149 | 6.163 | −9.068 | −17.300 | 1.00 | 70.56 |
| ATOM | 5877 | CA | MSE | 3149 | 6.879 | −10.362 | −17.117 | 1.00 | 70.95 |
| ATOM | 5878 | N | PRO | 3150 | 6.168 | −11.313 | −14.973 | 1.00 | 68.78 |
| ATOM | 5879 | CD | PRO | 3150 | 5.019 | −11.963 | −15.618 | 1.00 | 68.56 |
| ATOM | 5880 | CA | PRO | 3150 | 6.209 | −11.660 | −13.551 | 1.00 | 67.47 |
| ATOM | 5881 | CB | PRO | 3150 | 4.956 | −12.508 | −13.373 | 1.00 | 67.99 |
| ATOM | 5882 | CG | PRO | 3150 | 4.794 | −13.147 | −14.724 | 1.00 | 68.42 |
| ATOM | 5883 | C | PRO | 3150 | 7.463 | −12.398 | −13.105 | 1.00 | 66.14 |
| ATOM | 5884 | O | PRO | 3150 | 7.832 | −13.427 | −13.679 | 1.00 | 66.42 |
| ATOM | 5885 | N | VAL | 3151 | 8.105 | −11.863 | −12.068 | 1.00 | 64.31 |
| ATOM | 5886 | CA | VAL | 3151 | 9.324 | −12.445 | −11.508 | 1.00 | 62.30 |
| ATOM | 5887 | CB | VAL | 3151 | 10.571 | −11.607 | −11.875 | 1.00 | 62.18 |
| ATOM | 5888 | CG1 | VAL | 3151 | 11.817 | −12.299 | −11.372 | 1.00 | 61.97 |
| ATOM | 5889 | CG2 | VAL | 3151 | 10.648 | −11.398 | −13.374 | 1.00 | 62.53 |
| ATOM | 5890 | C | VAL | 3151 | 9.247 | −12.502 | −9.981 | 1.00 | 60.85 |
| ATOM | 5891 | O | VAL | 3151 | 9.061 | −11.472 | −9.326 | 1.00 | 60.38 |
| ATOM | 5892 | N | ALA | 3152 | 9.387 | −13.703 | −9.421 | 1.00 | 59.03 |
| ATOM | 5893 | CA | ALA | 3152 | 9.360 | −13.880 | −7.973 | 1.00 | 57.10 |
| ATOM | 5894 | CB | ALA | 3152 | 9.332 | −15.353 | −7.623 | 1.00 | 56.77 |
| ATOM | 5895 | C | ALA | 3152 | 10.620 | −13.231 | −7.409 | 1.00 | 55.85 |
| ATOM | 5896 | O | ALA | 3152 | 11.694 | −13.328 | −8.003 | 1.00 | 55.28 |
| ATOM | 5897 | N | PRO | 3153 | 10.508 | −12.577 | −6.242 | 1.00 | 54.78 |
| ATOM | 5898 | CD | PRO | 3153 | 9.335 | −12.590 | −5.354 | 1.00 | 54.73 |
| ATOM | 5899 | CA | PRO | 3153 | 11.635 | −11.901 | −5.592 | 1.00 | 53.81 |
| ATOM | 5900 | CB | PRO | 3153 | 11.059 | −11.498 | −4.234 | 1.00 | 54.39 |
| ATOM | 5901 | CG | PRO | 3153 | 9.973 | −12.507 | −4.002 | 1.00 | 54.71 |
| ATOM | 5902 | C | PRO | 3153 | 12.939 | −12.691 | −5.479 | 1.00 | 52.78 |
| ATOM | 5903 | O | PRO | 3153 | 12.939 | −13.894 | −5.226 | 1.00 | 52.55 |
| ATOM | 5904 | N | TYR | 3154 | 14.050 | −11.990 | −5.686 | 1.00 | 51.29 |
| ATOM | 5905 | CA | TYR | 3154 | 15.378 | −12.585 | −5.608 | 1.00 | 50.25 |
| ATOM | 5906 | CB | TYR | 3154 | 15.807 | −13.111 | −6.972 | 1.00 | 50.65 |
| ATOM | 5907 | CG | TYR | 3154 | 15.944 | −12.025 | −8.022 | 1.00 | 50.79 |
| ATOM | 5908 | CD1 | TYR | 3154 | 14.815 | −11.419 | −8.577 | 1.00 | 50.42 |
| ATOM | 5909 | CE1 | TYR | 3154 | 14.932 | −10.428 | −9.550 | 1.00 | 50.44 |
| ATOM | 5910 | CD2 | TYR | 3154 | 17.200 | −11.607 | −8.464 | 1.00 | 49.96 |
| ATOM | 5911 | CE2 | TYR | 3154 | 17.327 | −10.618 | −9.438 | 1.00 | 49.98 |
| ATOM | 5912 | CZ | TYR | 3154 | 16.188 | −10.034 | −9.977 | 1.00 | 50.51 |
| ATOM | 5913 | OH | TYR | 3154 | 16.296 | −9.070 | −10.956 | 1.00 | 50.89 |
| ATOM | 5914 | C | TYR | 3154 | 16.393 | −11.542 | −5.144 | 1.00 | 49.70 |
| ATOM | 5915 | O | TYR | 3154 | 16.210 | −10.346 | −5.365 | 1.00 | 49.15 |
| ATOM | 5916 | N | TRP | 3155 | 17.465 | −12.001 | −4.508 | 1.00 | 49.09 |
| ATOM | 5917 | CA | TRP | 3155 | 18.504 | −11.103 | −4.022 | 1.00 | 49.21 |
| ATOM | 5918 | CB | TRP | 3155 | 19.464 | −11.837 | −3.085 | 1.00 | 48.23 |
| ATOM | 5919 | CG | TRP | 3155 | 18.808 | −12.485 | −1.925 | 1.00 | 47.22 |
| ATOM | 5920 | CD2 | TRP | 3155 | 17.904 | −11.878 | −0.996 | 1.00 | 46.64 |
| ATOM | 5921 | CE2 | TRP | 3155 | 17.514 | −12.878 | −0.081 | 1.00 | 46.72 |
| ATOM | 5922 | CE3 | TRP | 3155 | 17.383 | −10.586 | −0.847 | 1.00 | 46.86 |
| ATOM | 5923 | CD1 | TRP | 3155 | 18.931 | −13.785 | −1.547 | 1.00 | 46.98 |
| ATOM | 5924 | NE1 | TRP | 3155 | 18.157 | −14.033 | −0.443 | 1.00 | 46.90 |
| ATOM | 5925 | CZ2 | TRP | 3155 | 16.622 | −12.630 | 0.972 | 1.00 | 46.50 |
| ATOM | 5926 | CZ3 | TRP | 3155 | 16.495 | −10.339 | 0.203 | 1.00 | 46.39 |
| ATOM | 5927 | CH2 | TRP | 3155 | 16.126 | −11.359 | 1.096 | 1.00 | 46.38 |
| ATOM | 5928 | C | TRP | 3155 | 19.296 | −10.590 | −5.207 | 1.00 | 50.04 |
| ATOM | 5929 | O | TRP | 3155 | 19.629 | −11.359 | −6.101 | 1.00 | 50.56 |
| ATOM | 5930 | N | THR | 3156 | 19.605 | −9.298 | −5.214 | 1.00 | 50.73 |
| ATOM | 5931 | CA | THR | 3156 | 20.372 | −8.720 | −6.308 | 1.00 | 51.26 |
| ATOM | 5932 | CB | THR | 3156 | 19.880 | −7.318 | −6.674 | 1.00 | 50.88 |
| ATOM | 5933 | OG1 | THR | 3156 | 20.221 | −6.400 | −5.628 | 1.00 | 50.70 |
| ATOM | 5934 | CG2 | THR | 3156 | 18.379 | −7.326 | −6.869 | 1.00 | 51.27 |
| ATOM | 5935 | C | THR | 3156 | 21.842 | −8.615 | −5.941 | 1.00 | 52.24 |
| ATOM | 5936 | O | THR | 3156 | 22.700 | −8.550 | −6.819 | 1.00 | 52.33 |
| ATOM | 5937 | N | SER | 3157 | 22.135 | −8.592 | −4.643 | 1.00 | 53.52 |
| ATOM | 5938 | CA | SER | 3157 | 23.520 | −8.497 | −4.176 | 1.00 | 54.58 |
| ATOM | 5939 | CB | SER | 3157 | 23.845 | −7.072 | −3.722 | 1.00 | 53.89 |
| ATOM | 5940 | OG | SER | 3157 | 23.718 | −6.153 | −4.789 | 1.00 | 53.44 |
| ATOM | 5941 | C | SER | 3157 | 23.769 | −9.456 | −3.022 | 1.00 | 55.64 |
| ATOM | 5942 | O | SER | 3157 | 24.179 | −9.045 | −1.937 | 1.00 | 55.63 |
| ATOM | 5943 | N | PRO | 3158 | 23.540 | −10.760 | −3.250 | 1.00 | 56.77 |
| ATOM | 5944 | CD | PRO | 3158 | 23.396 | −11.403 | −4.567 | 1.00 | 56.59 |
| ATOM | 5945 | CA | PRO | 3158 | 23.748 | −11.764 | −2.201 | 1.00 | 57.49 |
| ATOM | 5946 | CB | PRO | 3158 | 23.533 | −13.080 | −2.941 | 1.00 | 57.10 |
| ATOM | 5947 | CG | PRO | 3158 | 24.024 | −12.755 | −4.323 | 1.00 | 57.31 |
| ATOM | 5948 | C | PRO | 3158 | 25.146 | −11.661 | −1.605 | 1.00 | 58.38 |
| ATOM | 5949 | O | PRO | 3158 | 25.388 | −12.070 | −0.468 | 1.00 | 58.22 |
| ATOM | 5950 | N | GLU | 3159 | 26.067 | −11.106 | −2.386 | 1.00 | 59.59 |
| ATOM | 5951 | CA | GLU | 3159 | 27.435 | −10.954 | −1.928 | 1.00 | 60.72 |
| ATOM | 5952 | CB | GLU | 3159 | 28.321 | −10.439 | −3.062 | 1.00 | 62.53 |
| ATOM | 5953 | CG | GLU | 3159 | 28.488 | −11.471 | −4.168 | 1.00 | 65.72 |
| ATOM | 5954 | CD | GLU | 3159 | 28.981 | −12.822 | −3.633 | 1.00 | 67.62 |
| ATOM | 5955 | OE1 | GLU | 3159 | 30.215 | −13.004 | −3.473 | 1.00 | 68.34 |
| ATOM | 5956 | OE2 | GLU | 3159 | 28.125 | −13.699 | −3.361 | 1.00 | 68.68 |
| ATOM | 5957 | C | GLU | 3159 | 27.516 | −10.035 | −0.717 | 1.00 | 60.39 |
| ATOM | 5958 | O | GLU | 3159 | 28.267 | −10.299 | 0.212 | 1.00 | 60.66 |
| ATOM | 5959 | N | LYS | 3160 | 26.726 | −8.967 | −0.718 | 1.00 | 59.69 |
| ATOM | 5960 | CA | LYS | 3160 | 26.720 | −8.014 | 0.393 | 1.00 | 58.90 |
| ATOM | 5961 | CB | LYS | 3160 | 26.156 | −6.672 | −0.065 | 1.00 | 59.09 |
| ATOM | 5962 | CG | LYS | 3160 | 26.800 | −6.102 | −1.297 | 1.00 | 60.62 |
| ATOM | 5963 | CD | LYS | 3160 | 26.281 | −4.701 | −1.560 | 1.00 | 61.12 |
| ATOM | 5964 | CE | LYS | 3160 | 27.051 | −4.034 | −2.683 | 1.00 | 61.67 |
| ATOM | 5965 | NZ | LYS | 3160 | 26.631 | −2.618 | −2.824 | 1.00 | 62.24 |
| ATOM | 5966 | C | LYS | 3160 | 25.870 | −8.471 | 1.570 | 1.00 | 58.15 |
| ATOM | 5967 | O | LYS | 3160 | 25.566 | −7.673 | 2.460 | 1.00 | 58.16 |
| ATOM | 5968 | N | MSE | 3161 | 25.455 | −9.730 | 1.568 | 1.00 | 57.03 |
| ATOM | 5969 | CA | MSE | 3161 | 24.624 | −10.243 | 2.653 | 1.00 | 56.02 |
| ATOM | 5970 | CB | MSE | 3161 | 23.302 | −10.766 | 2.089 | 1.00 | 54.94 |
| ATOM | 5971 | CG | MSE | 3161 | 22.436 | −9.683 | 1.463 | 1.00 | 53.02 |
| ATOM | 5972 | SE | MSE | 3161 | 20.924 | −10.315 | 0.724 | 1.00 | 52.22 |
| ATOM | 5973 | CE | MSE | 3161 | 19.989 | −10.802 | 2.180 | 1.00 | 50.52 |
| ATOM | 5974 | C | MSE | 3161 | 25.372 | −11.349 | 3.381 | 1.00 | 56.09 |
| ATOM | 5975 | O | MSE | 3161 | 24.787 | −12.130 | 4.135 | 1.00 | 56.36 |
| ATOM | 5976 | N | GLU | 3162 | 26.682 | −11.387 | 3.146 | 1.00 | 56.01 |
| ATOM | 5977 | CA | GLU | 3162 | 27.584 | −12.375 | 3.736 | 1.00 | 55.93 |
| ATOM | 5978 | CB | GLU | 3162 | 29.021 | −12.131 | 3.256 | 1.00 | 57.48 |
| ATOM | 5979 | CG | GLU | 3162 | 29.216 | −12.008 | 1.759 | 1.00 | 59.23 |
| ATOM | 5980 | CD | GLU | 3162 | 29.309 | −13.341 | 1.055 | 1.00 | 60.27 |
| ATOM | 5981 | OE1 | GLU | 3162 | 28.448 | −14.210 | 1.318 | 1.00 | 61.01 |
| ATOM | 5982 | OE2 | GLU | 3162 | 30.241 | −13.509 | 0.233 | 1.00 | 60.72 |
| ATOM | 5983 | C | GLU | 3162 | 27.607 | −12.319 | 5.262 | 1.00 | 55.10 |
| ATOM | 5984 | O | GLU | 3162 | 27.311 | −13.301 | 5.945 | 1.00 | 54.98 |
| ATOM | 5985 | N | LYS | 3163 | 27.996 | −11.156 | 5.777 | 1.00 | 53.85 |
| ATOM | 5986 | CA | LYS | 3163 | 28.120 | −10.913 | 7.209 | 1.00 | 51.81 |
| ATOM | 5987 | CB | LYS | 3163 | 28.721 | −9.521 | 7.419 | 1.00 | 51.93 |
| ATOM | 5988 | CG | LYS | 3163 | 28.979 | −9.121 | 8.856 | 1.00 | 51.89 |
| ATOM | 5989 | CD | LYS | 3163 | 29.409 | −7.668 | 8.877 | 1.00 | 52.70 |
| ATOM | 5990 | CE | LYS | 3163 | 29.482 | −7.099 | 10.275 | 1.00 | 53.18 |
| ATOM | 5991 | NZ | LYS | 3163 | 29.645 | −5.613 | 10.231 | 1.00 | 53.09 |
| ATOM | 5992 | C | LYS | 3163 | 26.793 | −11.041 | 7.953 | 1.00 | 50.72 |
| ATOM | 5993 | O | LYS | 3163 | 25.949 | −10.142 | 7.901 | 1.00 | 50.83 |
| ATOM | 5994 | N | LYS | 3164 | 26.610 | −12.159 | 8.650 | 1.00 | 49.06 |
| ATOM | 5995 | CA | LYS | 3164 | 25.376 | −12.372 | 9.398 | 1.00 | 47.64 |
| ATOM | 5996 | CB | LYS | 3164 | 25.025 | −13.860 | 9.472 | 1.00 | 48.18 |
| ATOM | 5997 | CG | LYS | 3164 | 24.025 | −14.280 | 8.393 | 1.00 | 49.89 |
| ATOM | 5998 | CD | LYS | 3164 | 23.779 | −15.790 | 8.373 | 1.00 | 50.41 |
| ATOM | 5999 | CE | LYS | 3164 | 22.787 | −16.173 | 7.273 | 1.00 | 50.75 |
| ATOM | 6000 | NZ | LYS | 3164 | 22.722 | −17.646 | 7.033 | 1.00 | 51.00 |
| ATOM | 6001 | C | LYS | 3164 | 25.399 | −11.768 | 10.794 | 1.00 | 45.99 |
| ATOM | 6002 | O | LYS | 3164 | 24.393 | −11.225 | 11.243 | 1.00 | 46.02 |
| ATOM | 6003 | N | LEU | 3165 | 26.536 | −11.856 | 11.484 | 1.00 | 43.95 |
| ATOM | 6004 | CA | LEU | 3165 | 26.630 | −11.288 | 12.825 | 1.00 | 41.68 |
| ATOM | 6005 | CB | LEU | 3165 | 27.530 | −12.127 | 13.735 | 1.00 | 41.41 |
| ATOM | 6006 | CG | LEU | 3165 | 27.798 | −11.537 | 15.137 | 1.00 | 41.80 |
| ATOM | 6007 | CD1 | LEU | 3165 | 26.528 | −11.485 | 15.967 | 1.00 | 40.75 |
| ATOM | 6008 | CD2 | LEU | 3165 | 28.820 | −12.385 | 15.855 | 1.00 | 41.95 |
| ATOM | 6009 | C | LEU | 3165 | 27.184 | −9.885 | 12.781 | 1.00 | 40.60 |
| ATOM | 6010 | O | LEU | 3165 | 28.268 | −9.654 | 12.251 | 1.00 | 40.14 |
| ATOM | 6011 | N | HIS | 3166 | 26.426 | −8.943 | 13.331 | 1.00 | 39.25 |
| ATOM | 6012 | CA | HIS | 3166 | 26.869 | −7.558 | 13.396 | 1.00 | 37.80 |
| ATOM | 6013 | CB | HIS | 3166 | 25.798 | −6.594 | 12.888 | 1.00 | 40.57 |
| ATOM | 6014 | CG | HIS | 3166 | 25.774 | −6.410 | 11.404 | 1.00 | 43.08 |
| ATOM | 6015 | CD2 | HIS | 3166 | 25.865 | −5.289 | 10.648 | 1.00 | 44.64 |
| ATOM | 6016 | ND1 | HIS | 3166 | 25.566 | −7.451 | 10.522 | 1.00 | 44.80 |
| ATOM | 6017 | CE1 | HIS | 3166 | 25.529 | −6.979 | 9.286 | 1.00 | 45.27 |
| ATOM | 6018 | NE2 | HIS | 3166 | 25.707 | −5.669 | 9.334 | 1.00 | 45.69 |
| ATOM | 6019 | C | HIS | 3166 | 27.108 | −7.269 | 14.875 | 1.00 | 36.04 |
| ATOM | 6020 | O | HIS | 3166 | 26.167 | −7.230 | 15.669 | 1.00 | 35.15 |
| ATOM | 6021 | N | ALA | 3167 | 28.365 | −7.097 | 15.255 | 1.00 | 34.29 |
| ATOM | 6022 | CA | ALA | 3167 | 28.677 | −6.791 | 16.644 | 1.00 | 32.48 |
| ATOM | 6023 | CB | ALA | 3167 | 29.688 | −7.777 | 17.200 | 1.00 | 32.36 |
| ATOM | 6024 | C | ALA | 3167 | 29.239 | −5.384 | 16.648 | 1.00 | 31.39 |
| ATOM | 6025 | O | ALA | 3167 | 30.135 | −5.063 | 15.855 | 1.00 | 31.82 |
| ATOM | 6026 | N | VAL | 3168 | 28.704 | −4.542 | 17.526 | 1.00 | 29.57 |
| ATOM | 6027 | CA | VAL | 3168 | 29.134 | −3.156 | 17.595 | 1.00 | 27.71 |
| ATOM | 6028 | CB | VAL | 3168 | 28.226 | −2.278 | 16.685 | 1.00 | 27.49 |
| ATOM | 6029 | CG1 | VAL | 3168 | 28.195 | −2.842 | 15.272 | 1.00 | 26.64 |
| ATOM | 6030 | CG2 | VAL | 3168 | 26.811 | −2.239 | 17.239 | 1.00 | 26.75 |
| ATOM | 6031 | C | VAL | 3168 | 29.070 | −2.615 | 19.018 | 1.00 | 26.78 |
| ATOM | 6032 | O | VAL | 3168 | 28.329 | −3.136 | 19.847 | 1.00 | 26.51 |
| ATOM | 6033 | N | PRO | 3169 | 29.860 | −1.571 | 19.322 | 1.00 | 25.75 |
| ATOM | 6034 | CD | PRO | 3169 | 30.703 | −0.764 | 18.434 | 1.00 | 26.17 |
| ATOM | 6035 | CA | PRO | 3169 | 29.841 | −0.990 | 20.663 | 1.00 | 25.63 |
| ATOM | 6036 | CB | PRO | 3169 | 31.012 | −0.015 | 20.660 | 1.00 | 25.34 |
| ATOM | 6037 | CG | PRO | 3169 | 31.725 | −0.253 | 19.382 | 1.00 | 26.02 |
| ATOM | 6038 | C | PRO | 3169 | 28.531 | −0.213 | 20.730 | 1.00 | 26.09 |
| ATOM | 6039 | O | PRO | 3169 | 27.948 | 0.107 | 19.693 | 1.00 | 25.98 |
| ATOM | 6040 | N | ALA | 3170 | 28.074 | 0.102 | 21.933 | 1.00 | 26.30 |
| ATOM | 6041 | CA | ALA | 3170 | 26.832 | 0.862 | 22.088 | 1.00 | 26.78 |
| ATOM | 6042 | CB | ALA | 3170 | 26.484 | 1.017 | 23.564 | 1.00 | 26.53 |
| ATOM | 6043 | C | ALA | 3170 | 26.983 | 2.235 | 21.454 | 1.00 | 26.52 |
| ATOM | 6044 | O | ALA | 3170 | 28.101 | 2.734 | 21.307 | 1.00 | 27.54 |
| ATOM | 6045 | N | ALA | 3171 | 25.854 | 2.818 | 21.058 | 1.00 | 26.13 |
| ATOM | 6046 | CA | ALA | 3171 | 25.790 | 4.148 | 20.450 | 1.00 | 26.58 |
| ATOM | 6047 | CB | ALA | 3171 | 26.711 | 5.116 | 21.181 | 1.00 | 26.27 |
| ATOM | 6048 | C | ALA | 3171 | 26.087 | 4.202 | 18.963 | 1.00 | 27.77 |
| ATOM | 6049 | O | ALA | 3171 | 25.857 | 5.226 | 18.312 | 1.00 | 28.62 |
| ATOM | 6050 | N | LYS | 3172 | 26.599 | 3.115 | 18.412 | 1.00 | 28.34 |
| ATOM | 6051 | CA | LYS | 3172 | 26.906 | 3.097 | 16.995 | 1.00 | 29.17 |
| ATOM | 6052 | CB | LYS | 3172 | 27.845 | 1.923 | 16.696 | 1.00 | 30.78 |
| ATOM | 6053 | CG | LYS | 3172 | 28.457 | 1.956 | 15.304 | 1.00 | 32.97 |
| ATOM | 6054 | CD | LYS | 3172 | 29.358 | 0.753 | 15.044 | 1.00 | 33.41 |
| ATOM | 6055 | CE | LYS | 3172 | 30.144 | 0.941 | 13.746 | 1.00 | 34.59 |
| ATOM | 6056 | NZ | LYS | 3172 | 29.263 | 1.283 | 12.577 | 1.00 | 35.03 |
| ATOM | 6057 | C | LYS | 3172 | 25.624 | 2.987 | 16.159 | 1.00 | 29.07 |
| ATOM | 6058 | O | LYS | 3172 | 24.585 | 2.528 | 16.647 | 1.00 | 29.22 |
| ATOM | 6059 | N | THR | 3173 | 25.690 | 3.432 | 14.909 | 1.00 | 28.75 |
| ATOM | 6060 | CA | THR | 3173 | 24.535 | 3.338 | 14.033 | 1.00 | 28.80 |
| ATOM | 6061 | CB | THR | 3173 | 24.457 | 4.545 | 13.109 | 1.00 | 28.30 |
| ATOM | 6062 | OG1 | THR | 3173 | 23.941 | 5.653 | 13.857 | 1.00 | 27.79 |
| ATOM | 6063 | CG2 | THR | 3173 | 23.543 | 4.260 | 11.928 | 1.00 | 28.83 |
| ATOM | 6064 | C | THR | 3173 | 24.645 | 2.032 | 13.258 | 1.00 | 29.42 |
| ATOM | 6065 | O | THR | 3173 | 25.713 | 1.681 | 12.755 | 1.00 | 30.21 |
| ATOM | 6066 | N | VAL | 3174 | 23.548 | 1.291 | 13.197 | 1.00 | 29.82 |
| ATOM | 6067 | CA | VAL | 3174 | 23.557 | 0.008 | 12.510 | 1.00 | 30.09 |
| ATOM | 6068 | CB | VAL | 3174 | 23.186 | −1.124 | 13.484 | 1.00 | 28.87 |
| ATOM | 6069 | CG1 | VAL | 3174 | 22.983 | −2.428 | 12.740 | 1.00 | 28.04 |
| ATOM | 6070 | CG2 | VAL | 3174 | 24.293 | −1.283 | 14.500 | 1.00 | 28.83 |
| ATOM | 6071 | C | VAL | 3174 | 22.619 | 0.013 | 11.318 | 1.00 | 30.91 |
| ATOM | 6072 | O | VAL | 3174 | 21.512 | 0.549 | 11.381 | 1.00 | 30.79 |
| ATOM | 6073 | N | LYS | 3175 | 23.076 | −0.578 | 10.222 | 1.00 | 32.06 |
| ATOM | 6074 | CA | LYS | 3175 | 22.267 | −0.628 | 9.024 | 1.00 | 33.55 |
| ATOM | 6075 | CB | LYS | 3175 | 22.770 | 0.408 | 8.012 | 1.00 | 34.53 |
| ATOM | 6076 | CG | LYS | 3175 | 21.870 | 0.577 | 6.801 | 1.00 | 36.89 |
| ATOM | 6077 | CD | LYS | 3175 | 22.337 | 1.726 | 5.924 | 1.00 | 39.24 |
| ATOM | 6078 | CE | LYS | 3175 | 21.393 | 1.951 | 4.750 | 1.00 | 39.73 |
| ATOM | 6079 | NZ | LYS | 3175 | 21.784 | 3.133 | 3.930 | 1.00 | 41.45 |
| ATOM | 6080 | C | LYS | 3175 | 22.249 | −2.024 | 8.407 | 1.00 | 33.32 |
| ATOM | 6081 | O | LYS | 3175 | 23.286 | −2.601 | 8.107 | 1.00 | 33.70 |
| ATOM | 6082 | N | PHE | 3176 | 21.050 | −2.558 | 8.235 | 1.00 | 33.92 |
| ATOM | 6083 | CA | PHE | 3176 | 20.865 | −3.873 | 7.650 | 1.00 | 34.13 |
| ATOM | 6084 | CB | PHE | 3176 | 19.968 | −4.721 | 8.540 | 1.00 | 31.96 |
| ATOM | 6085 | CG | PHE | 3176 | 20.587 | −5.082 | 9.845 | 1.00 | 30.12 |
| ATOM | 6086 | CD1 | PHE | 3176 | 21.910 | −5.497 | 9.906 | 1.00 | 29.71 |
| ATOM | 6087 | CD2 | PHE | 3176 | 19.838 | −5.071 | 11.005 | 1.00 | 29.63 |
| ATOM | 6088 | CE1 | PHE | 3176 | 22.478 | −5.904 | 11.102 | 1.00 | 29.50 |
| ATOM | 6089 | CE2 | PHE | 3176 | 20.399 | −5.476 | 12.210 | 1.00 | 30.45 |
| ATOM | 6090 | CZ | PHE | 3176 | 21.727 | −5.896 | 12.257 | 1.00 | 29.17 |
| ATOM | 6091 | C | PHE | 3176 | 20.197 | −3.704 | 6.300 | 1.00 | 35.61 |
| ATOM | 6092 | O | PHE | 3176 | 19.304 | −2.867 | 6.147 | 1.00 | 36.07 |
| ATOM | 6093 | N | LYS | 3177 | 20.625 | −4.483 | 5.315 | 1.00 | 36.95 |
| ATOM | 6094 | CA | LYS | 3177 | 20.002 | −4.383 | 4.014 | 1.00 | 38.56 |
| ATOM | 6095 | CB | LYS | 3177 | 20.748 | −3.380 | 3.125 | 1.00 | 39.62 |
| ATOM | 6096 | CG | LYS | 3177 | 22.254 | −3.523 | 3.060 | 1.00 | 41.82 |
| ATOM | 6097 | CD | LYS | 3177 | 22.844 | −2.304 | 2.342 | 1.00 | 42.64 |
| ATOM | 6098 | CE | LYS | 3177 | 24.365 | −2.276 | 2.402 | 1.00 | 44.37 |
| ATOM | 6099 | NZ | LYS | 3177 | 24.905 | −1.054 | 1.707 | 1.00 | 45.40 |
| ATOM | 6100 | C | LYS | 3177 | 19.841 | −5.721 | 3.322 | 1.00 | 38.93 |
| ATOM | 6101 | O | LYS | 3177 | 20.598 | −6.657 | 3.551 | 1.00 | 37.98 |
| ATOM | 6102 | N | CYS | 3178 | 18.806 | −5.799 | 2.498 | 1.00 | 40.12 |
| ATOM | 6103 | CA | CYS | 3178 | 18.495 | −6.999 | 1.748 | 1.00 | 41.37 |
| ATOM | 6104 | CB | CYS | 3178 | 17.349 | −7.739 | 2.427 | 1.00 | 42.64 |
| ATOM | 6105 | SG | CYS | 3178 | 17.858 | −8.494 | 3.970 | 1.00 | 44.96 |
| ATOM | 6106 | C | CYS | 3178 | 18.112 | −6.600 | 0.335 | 1.00 | 41.53 |
| ATOM | 6107 | O | CYS | 3178 | 16.950 | −6.693 | −0.061 | 1.00 | 41.06 |
| ATOM | 6108 | N | PRO | 3179 | 19.095 | −6.122 | −0.438 | 1.00 | 42.05 |
| ATOM | 6109 | CD | PRO | 3179 | 20.524 | −6.009 | −0.099 | 1.00 | 41.96 |
| ATOM | 6110 | CA | PRO | 3179 | 18.842 | −5.706 | −1.816 | 1.00 | 42.44 |
| ATOM | 6111 | CB | PRO | 3179 | 20.218 | −5.259 | −2.297 | 1.00 | 42.84 |
| ATOM | 6112 | CG | PRO | 3179 | 21.167 | −6.059 | −1.446 | 1.00 | 42.62 |
| ATOM | 6113 | C | PRO | 3179 | 18.252 | −6.840 | −2.645 | 1.00 | 43.20 |
| ATOM | 6114 | O | PRO | 3179 | 18.834 | −7.924 | −2.760 | 1.00 | 42.85 |
| ATOM | 6115 | N | SER | 3180 | 17.070 | −6.595 | −3.193 | 1.00 | 43.70 |
| ATOM | 6116 | CA | SER | 3180 | 16.421 | −7.594 | −4.010 | 1.00 | 45.13 |
| ATOM | 6117 | CB | SER | 3180 | 15.563 | −8.523 | −3.142 | 1.00 | 45.23 |
| ATOM | 6118 | OG | SER | 3180 | 14.694 | −7.799 | −2.301 | 1.00 | 45.85 |
| ATOM | 6119 | C | SER | 3180 | 15.589 | −6.957 | −5.109 | 1.00 | 45.73 |
| ATOM | 6120 | O | SER | 3180 | 15.593 | −5.737 | −5.290 | 1.00 | 45.62 |
| ATOM | 6121 | N | SER | 3181 | 14.892 | −7.796 | −5.861 | 1.00 | 46.63 |
| ATOM | 6122 | CA | SER | 3181 | 14.068 | −7.312 | −6.947 | 1.00 | 47.56 |
| ATOM | 6123 | CB | SER | 3181 | 14.935 | −7.013 | −8.163 | 1.00 | 47.39 |
| ATOM | 6124 | OG | SER | 3181 | 14.141 | −6.509 | −9.217 | 1.00 | 48.09 |
| ATOM | 6125 | C | SER | 3181 | 13.012 | −8.337 | −7.317 | 1.00 | 48.28 |
| ATOM | 6126 | O | SER | 3181 | 12.908 | −9.396 | −6.700 | 1.00 | 48.27 |
| ATOM | 6127 | N | GLY | 3182 | 12.233 | −8.010 | −8.338 | 1.00 | 49.22 |
| ATOM | 6128 | CA | GLY | 3182 | 11.183 | −8.897 | −8.788 | 1.00 | 49.41 |
| ATOM | 6129 | C | GLY | 3182 | 10.032 | −8.069 | −9.310 | 1.00 | 49.66 |
| ATOM | 6130 | O | GLY | 3182 | 9.958 | −6.870 | −9.055 | 1.00 | 49.58 |
| ATOM | 6131 | N | THR | 3183 | 9.132 | −8.698 | −10.051 | 1.00 | 49.75 |
| ATOM | 6132 | CA | THR | 3183 | 7.995 | −7.978 | −10.580 | 1.00 | 49.39 |
| ATOM | 6133 | CB | THR | 3183 | 8.222 | −7.586 | −12.045 | 1.00 | 49.92 |
| ATOM | 6134 | OG1 | THR | 3183 | 8.834 | −8.676 | −12.744 | 1.00 | 51.32 |
| ATOM | 6135 | CG2 | THR | 3183 | 9.128 | −6.367 | −12.127 | 1.00 | 50.52 |
| ATOM | 6136 | C | THR | 3183 | 6.710 | −8.775 | −10.435 | 1.00 | 49.32 |
| ATOM | 6137 | O | THR | 3183 | 6.652 | −9.957 | −10.766 | 1.00 | 49.83 |
| ATOM | 6138 | N | PRO | 3184 | 5.660 | −8.133 | −9.905 | 1.00 | 49.09 |
| ATOM | 6139 | CD | PRO | 3184 | 4.331 | −8.704 | −9.627 | 1.00 | 48.77 |
| ATOM | 6140 | CA | PRO | 3184 | 5.725 | −6.733 | −9.475 | 1.00 | 49.44 |
| ATOM | 6141 | CB | PRO | 3184 | 4.294 | −6.440 | −9.027 | 1.00 | 48.98 |
| ATOM | 6142 | CG | PRO | 3184 | 3.802 | −7.772 | −8.563 | 1.00 | 49.44 |
| ATOM | 6143 | C | PRO | 3184 | 6.748 | −6.502 | −8.364 | 1.00 | 49.66 |
| ATOM | 6144 | O | PRO | 3184 | 7.232 | −7.449 | −7.737 | 1.00 | 49.77 |
| ATOM | 6145 | N | GLN | 3185 | 7.094 | −5.242 | −8.142 | 1.00 | 49.60 |
| ATOM | 6146 | CA | GLN | 3185 | 8.047 | −4.893 | −7.100 | 1.00 | 49.28 |
| ATOM | 6147 | CB | GLN | 3185 | 8.160 | −3.376 | −6.994 | 1.00 | 50.58 |
| ATOM | 6148 | CG | GLN | 3185 | 9.280 | −2.815 | −7.831 | 1.00 | 52.24 |
| ATOM | 6149 | CD | GLN | 3185 | 10.622 | −3.206 | −7.271 | 1.00 | 53.40 |
| ATOM | 6150 | OE1 | GLN | 3185 | 10.923 | −2.911 | −6.108 | 1.00 | 53.67 |
| ATOM | 6151 | NE2 | GLN | 3185 | 11.440 | −3.877 | −8.084 | 1.00 | 53.76 |
| ATOM | 6152 | C | GLN | 3185 | 7.591 | −5.473 | −5.769 | 1.00 | 48.11 |
| ATOM | 6153 | O | GLN | 3185 | 6.472 | −5.226 | −5.324 | 1.00 | 47.56 |
| ATOM | 6154 | N | PRO | 3186 | 8.455 | −6.260 | −5.117 | 1.00 | 47.13 |
| ATOM | 6155 | CD | PRO | 3186 | 9.761 | −6.733 | −5.611 | 1.00 | 46.44 |
| ATOM | 6156 | CA | PRO | 3186 | 8.121 | −6.872 | −3.830 | 1.00 | 46.33 |
| ATOM | 6157 | CB | PRO | 3186 | 9.160 | −7.978 | −3.709 | 1.00 | 46.35 |
| ATOM | 6158 | CG | PRO | 3186 | 10.350 | −7.379 | −4.388 | 1.00 | 46.47 |
| ATOM | 6159 | C | PRO | 3186 | 8.163 | −5.894 | −2.652 | 1.00 | 45.83 |
| ATOM | 6160 | O | PRO | 3186 | 8.907 | −4.917 | −2.679 | 1.00 | 46.33 |
| ATOM | 6161 | N | THR | 3187 | 7.352 | −6.152 | −1.630 | 1.00 | 44.81 |
| ATOM | 6162 | CA | THR | 3187 | 7.326 | −5.302 | −0.445 | 1.00 | 44.15 |
| ATOM | 6163 | CB | THR | 3187 | 5.988 | −5.390 | 0.315 | 1.00 | 44.90 |
| ATOM | 6164 | OG1 | THR | 3187 | 5.677 | −6.766 | 0.578 | 1.00 | 44.91 |
| ATOM | 6165 | CG2 | THR | 3187 | 4.870 | −4.727 | −0.482 | 1.00 | 44.43 |
| ATOM | 6166 | C | THR | 3187 | 8.420 | −5.744 | 0.507 | 1.00 | 43.57 |
| ATOM | 6167 | O | THR | 3187 | 8.958 | −6.847 | 0.381 | 1.00 | 43.92 |
| ATOM | 6168 | NL | LEU | 3188 | 8.733 | −4.889 | 1.475 | 1.00 | 42.52 |
| ATOM | 6169 | CA | LEU | 3188 | 9.782 | −5.181 | 2.447 | 1.00 | 41.35 |
| ATOM | 6170 | CB | LEU | 3188 | 11.045 | −4.382 | 2.110 | 1.00 | 41.22 |
| ATOM | 6171 | CG | LEU | 3188 | 12.415 | −4.644 | 2.744 | 1.00 | 40.47 |
| ATOM | 6172 | CD1 | LEU | 3188 | 13.088 | −3.304 | 2.899 | 1.00 | 40.89 |
| ATOM | 6173 | CD2 | LEU | 3188 | 12.325 | −5.333 | 4.078 | 1.00 | 40.46 |
| ATOM | 6174 | C | LEU | 3188 | 9.351 | −4.812 | 3.859 | 1.00 | 40.80 |
| ATOM | 6175 | O | LEU | 3188 | 9.027 | −3.656 | 4.140 | 1.00 | 40.72 |
| ATOM | 6176 | N | ARG | 3189 | 9.347 | −5.799 | 4.746 | 1.00 | 39.63 |
| ATOM | 6177 | CA | ARG | 3189 | 9.010 | −5.563 | 6.140 | 1.00 | 38.01 |
| ATOM | 6178 | CB | ARG | 3189 | 7.619 | −6.110 | 6.478 | 1.00 | 38.85 |
| ATOM | 6179 | CG | ARG | 3189 | 7.075 | −7.111 | 5.485 | 1.00 | 41.44 |
| ATOM | 6180 | CD | ARG | 3189 | 5.612 | −7.430 | 5.780 | 1.00 | 43.59 |
| ATOM | 6181 | NE | ARG | 3189 | 5.445 | −8.177 | 7.026 | 1.00 | 45.68 |
| ATOM | 6182 | CZ | ARG | 3189 | 5.626 | −9.490 | 7.149 | 1.00 | 46.11 |
| ATOM | 6183 | NH1 | ARG | 3189 | 5.977 | −10.218 | 6.099 | 1.00 | 46.28 |
| ATOM | 6184 | NH2 | ARG | 3189 | 5.459 | −10.076 | 8.329 | 1.00 | 46.31 |
| ATOM | 6185 | C | ARG | 3189 | 10.078 | −6.225 | 7.005 | 1.00 | 36.25 |
| ATOM | 6186 | O | ARG | 3189 | 10.679 | −7.230 | 6.612 | 1.00 | 34.65 |
| ATOM | 6187 | N | TRP | 3190 | 10.327 | −5.646 | 8.174 | 1.00 | 34.53 |
| ATOM | 6188 | CA | TRP | 3190 | 11.327 | −6.187 | 9.070 | 1.00 | 33.43 |
| ATOM | 6189 | CB | TRP | 3190 | 12.380 | −5.121 | 9.397 | 1.00 | 31.57 |
| ATOM | 6190 | CG | TRP | 3190 | 13.233 | −4.691 | 8.229 | 1.00 | 29.17 |
| ATOM | 6191 | CD2 | TRP | 3190 | 14.514 | −5.225 | 7.851 | 1.00 | 28.04 |
| ATOM | 6192 | CE2 | TRP | 3190 | 14.935 | −4.523 | 6.695 | 1.00 | 27.65 |
| ATOM | 6193 | CE3 | TRP | 3190 | 15.344 | −6.225 | 8.375 | 1.00 | 26.62 |
| ATOM | 6194 | CD1 | TRP | 3190 | 12.943 | −3.723 | 7.318 | 1.00 | 28.83 |
| ATOM | 6195 | NE1 | TRP | 3190 | 13.960 | −3.613 | 6.394 | 1.00 | 28.36 |
| ATOM | 6196 | CZ2 | TRP | 3190 | 16.150 | −4.790 | 6.052 | 1.00 | 27.20 |
| ATOM | 6197 | CZ3 | TRP | 3190 | 16.556 | −6.491 | 7.733 | 1.00 | 26.92 |
| ATOM | 6198 | CH2 | TRP | 3190 | 16.945 | −5.774 | 6.581 | 1.00 | 25.86 |
| ATOM | 6199 | C | TRP | 3190 | 10.737 | −6.727 | 10.364 | 1.00 | 34.15 |
| ATOM | 6200 | O | TRP | 3190 | 9.735 | −6.217 | 10.870 | 1.00 | 34.81 |
| ATOM | 6201 | N | LEU | 3191 | 11.371 | −7.764 | 10.899 | 1.00 | 34.65 |
| ATOM | 6202 | CA | LEU | 3191 | 10.936 | −8.362 | 12.154 | 1.00 | 34.72 |
| ATOM | 6203 | CB | LEU | 3191 | 10.528 | −9.819 | 11.934 | 1.00 | 34.93 |
| ATOM | 6204 | CG | LEU | 3191 | 9.397 | −10.101 | 10.952 | 1.00 | 36.10 |
| ATOM | 6205 | CD1 | LEU | 3191 | 9.245 | −11.606 | 10.823 | 1.00 | 35.62 |
| ATOM | 6206 | CD2 | LEU | 3191 | 8.099 | −9.453 | 11.426 | 1.00 | 35.50 |
| ATOM | 6207 | C | LEU | 3191 | 12.086 | −8.319 | 13.159 | 1.00 | 34.49 |
| ATOM | 6208 | O | LEU | 3191 | 13.256 | −8.316 | 12.783 | 1.00 | 33.52 |
| ATOM | 6209 | N | LYS | 3192 | 11.745 | −8.277 | 14.438 | 1.00 | 34.77 |
| ATOM | 6210 | CA | LYS | 3192 | 12.752 | −8.285 | 15.493 | 1.00 | 35.48 |
| ATOM | 6211 | CB | LYS | 3192 | 12.616 | −7.041 | 16.373 | 1.00 | 36.47 |
| ATOM | 6212 | CG | LYS | 3192 | 13.712 | −6.851 | 17.421 | 1.00 | 37.73 |
| ATOM | 6213 | CD | LYS | 3192 | 13.389 | −5.629 | 18.286 | 1.00 | 39.11 |
| ATOM | 6214 | CE | LYS | 3192 | 14.567 | −5.163 | 19.137 | 1.00 | 40.36 |
| ATOM | 6215 | NZ | LYS | 3192 | 15.004 | −6.165 | 20.143 | 1.00 | 40.52 |
| ATOM | 6216 | C | LYS | 3192 | 12.447 | −9.540 | 16.300 | 1.00 | 35.55 |
| ATOM | 6217 | O | LYS | 3192 | 11.407 | −9.631 | 16.950 | 1.00 | 35.10 |
| ATOM | 6218 | N | ASN | 3193 | 13.341 | −10.517 | 16.231 | 1.00 | 36.08 |
| ATOM | 6219 | CA | ASN | 3193 | 13.147 | −11.775 | 16.942 | 1.00 | 37.14 |
| ATOM | 6220 | CB | ASN | 3193 | 13.129 | −11.537 | 18.452 | 1.00 | 37.05 |
| ATOM | 6221 | CG | ASN | 3193 | 14.427 | −10.957 | 18.959 | 1.00 | 37.74 |
| ATOM | 6222 | OD1 | ASN | 3193 | 15.513 | −11.457 | 18.647 | 1.00 | 37.06 |
| ATOM | 6223 | ND2 | ASN | 3193 | 14.326 | −9.895 | 19.752 | 1.00 | 38.55 |
| ATOM | 6224 | C | ASN | 3193 | 11.860 | −12.485 | 16.514 | 1.00 | 37.53 |
| ATOM | 6225 | O | ASN | 3193 | 11.126 | −13.024 | 17.348 | 1.00 | 37.70 |
| ATOM | 6226 | N | GLY | 3194 | 11.592 | −12.465 | 15.209 | 1.00 | 37.86 |
| ATOM | 6227 | CA | GLY | 3194 | 10.416 | −13.122 | 14.669 | 1.00 | 38.13 |
| ATOM | 6228 | C | GLY | 3194 | 9.088 | −12.404 | 14.813 | 1.00 | 39.56 |
| ATOM | 6229 | O | GLY | 3194 | 8.095 | −12.814 | 14.206 | 1.00 | 39.58 |
| ATOM | 6230 | N | LYS | 3195 | 9.048 | −11.341 | 15.607 | 1.00 | 40.40 |
| ATOM | 6231 | CA | LYS | 3195 | 7.808 | −10.599 | 15.805 | 1.00 | 41.58 |
| ATOM | 6232 | CB | LYS | 3195 | 7.670 | −10.217 | 17.275 | 1.00 | 43.58 |
| ATOM | 6233 | CG | LYS | 3195 | 8.021 | −11.326 | 18.251 | 1.00 | 45.54 |
| ATOM | 6234 | CD | LYS | 3195 | 7.095 | −12.522 | 18.105 | 1.00 | 47.43 |
| ATOM | 6235 | CE | LYS | 3195 | 7.432 | −13.558 | 19.160 | 1.00 | 48.54 |
| ATOM | 6236 | NZ | LYS | 3195 | 7.577 | −12.896 | 20.493 | 1.00 | 49.21 |
| ATOM | 6237 | C | LYS | 3195 | 7.761 | −9.329 | 14.966 | 1.00 | 41.57 |
| ATOM | 6238 | O | LYS | 3195 | 8.744 | −8.946 | 14.341 | 1.00 | 41.99 |
| ATOM | 6239 | N | GLU | 3196 | 6.613 | −8.670 | 14.960 | 1.00 | 41.81 |
| ATOM | 6240 | CA | GLU | 3196 | 6.473 | −7.436 | 14.209 | 1.00 | 42.49 |
| ATOM | 6241 | CB | GLU | 3196 | 5.030 | −6.945 | 14.238 | 1.00 | 43.66 |
| ATOM | 6242 | CG | GLU | 3196 | 4.815 | −5.725 | 13.359 | 1.00 | 45.79 |
| ATOM | 6243 | CD | GLU | 3196 | 3.533 | −4.988 | 13.669 | 1.00 | 47.22 |
| ATOM | 6244 | OE1 | GLU | 3196 | 3.182 | −4.081 | 12.885 | 1.00 | 48.89 |
| ATOM | 6245 | OE2 | GLU | 3196 | 2.884 | −5.301 | 14.694 | 1.00 | 47.38 |
| ATOM | 6246 | C | GLU | 3196 | 7.360 | −6.379 | 14.859 | 1.00 | 42.26 |
| ATOM | 6247 | O | GLU | 3196 | 7.479 | −6.329 | 16.079 | 1.00 | 42.25 |
| ATOM | 6248 | N | PHE | 3197 | 7.966 | −5.523 | 14.044 | 1.00 | 41.72 |
| ATOM | 6249 | CA | PHE | 3197 | 8.844 | −4.478 | 14.560 | 1.00 | 40.76 |
| ATOM | 6250 | CB | PHE | 3197 | 10.252 | −4.686 | 13.984 | 1.00 | 39.76 |
| ATOM | 6251 | CG | PHE | 3197 | 11.335 | −3.881 | 14.662 | 1.00 | 38.89 |
| ATOM | 6252 | CD1 | PHE | 3197 | 11.335 | −3.688 | 16.043 | 1.00 | 38.85 |
| ATOM | 6253 | CD2 | PHE | 3197 | 12.385 | −3.347 | 13.914 | 1.00 | 38.27 |
| ATOM | 6254 | CE1 | PHE | 3197 | 12.366 | −2.973 | 16.672 | 1.00 | 37.34 |
| ATOM | 6255 | CE2 | PHE | 3197 | 13.424 | −2.630 | 14.536 | 1.00 | 38.68 |
| ATOM | 6256 | CZ | PHE | 3197 | 13.409 | −2.444 | 15.919 | 1.00 | 37.81 |
| ATOM | 6257 | C | PHE | 3197 | 8.294 | −3.108 | 14.182 | 1.00 | 40.60 |
| ATOM | 6258 | O | PHE | 3197 | 8.190 | −2.776 | 13.012 | 1.00 | 40.35 |
| ATOM | 6259 | N | LYS | 3198 | 7.918 | −2.321 | 15.181 | 1.00 | 41.31 |
| ATOM | 6260 | CA | LYS | 3198 | 7.390 | −0.982 | 14.934 | 1.00 | 42.03 |
| ATOM | 6261 | CB | LYS | 3198 | 6.026 | −0.787 | 15.609 | 1.00 | 43.37 |
| ATOM | 6262 | CG | LYS | 3198 | 4.866 | −1.536 | 14.974 | 1.00 | 44.87 |
| ATOM | 6263 | CD | LYS | 3198 | 3.558 | −1.226 | 15.704 | 1.00 | 46.03 |
| ATOM | 6264 | CE | LYS | 3198 | 2.467 | −2.238 | 15.356 | 1.00 | 46.93 |
| ATOM | 6265 | NZ | LYS | 3198 | 1.198 | −1.998 | 16.109 | 1.00 | 47.00 |
| ATOM | 6266 | C | LYS | 3198 | 8.353 | 0.055 | 15.483 | 1.00 | 41.33 |
| ATOM | 6267 | O | LYS | 3198 | 8.953 | −0.142 | 16.535 | 1.00 | 41.31 |
| ATOM | 6268 | N | PRO | 3199 | 8.505 | 1.181 | 14.778 | 1.00 | 40.99 |
| ATOM | 6269 | CD | PRO | 3199 | 7.770 | 1.579 | 13.571 | 1.00 | 41.26 |
| ATOM | 6270 | CA | PRO | 3199 | 9.407 | 2.251 | 15.210 | 1.00 | 40.86 |
| ATOM | 6271 | CB | PRO | 3199 | 9.091 | 3.393 | 14.240 | 1.00 | 41.01 |
| ATOM | 6272 | CG | PRO | 3199 | 7.713 | 3.071 | 13.739 | 1.00 | 41.45 |
| ATOM | 6273 | C | PRO | 3199 | 9.250 | 2.647 | 16.673 | 1.00 | 40.59 |
| ATOM | 6274 | O | PRO | 3199 | 10.213 | 3.048 | 17.311 | 1.00 | 40.53 |
| ATOM | 6275 | N | ASP | 3200 | 8.053 | 2.523 | 17.226 | 1.00 | 41.07 |
| ATOM | 6276 | CA | ASP | 3200 | 7.914 | 2.886 | 18.626 | 1.00 | 41.63 |
| ATOM | 6277 | CB | ASP | 3200 | 6.474 | 3.248 | 18.970 | 1.00 | 43.48 |
| ATOM | 6278 | CG | ASP | 3200 | 6.150 | 4.673 | 18.575 | 1.00 | 46.03 |
| ATOM | 6279 | OD1 | ASP | 3200 | 5.880 | 4.927 | 17.375 | 1.00 | 47.86 |
| ATOM | 6280 | OD2 | ASP | 3200 | 6.203 | 5.550 | 19.463 | 1.00 | 47.33 |
| ATOM | 6281 | C | ASP | 3200 | 8.465 | 1.849 | 19.591 | 1.00 | 40.73 |
| ATOM | 6282 | O | ASP | 3200 | 8.395 | 2.021 | 20.807 | 1.00 | 40.30 |
| ATOM | 6283 | N | HIS | 3201 | 9.040 | 0.782 | 19.052 | 1.00 | 39.81 |
| ATOM | 6284 | CA | HIS | 3201 | 9.625 | −0.239 | 19.906 | 1.00 | 39.26 |
| ATOM | 6285 | CB | HIS | 3201 | 9.797 | −1.562 | 19.167 | 1.00 | 39.84 |
| ATOM | 6286 | CG | HIS | 3201 | 8.508 | −2.254 | 18.868 | 1.00 | 40.22 |
| ATOM | 6287 | CD2 | HIS | 3201 | 8.210 | −3.247 | 17.999 | 1.00 | 39.97 |
| ATOM | 6288 | ND1 | HIS | 3201 | 7.329 | −1.932 | 19.505 | 1.00 | 40.01 |
| ATOM | 6289 | CE1 | HIS | 3201 | 6.358 | −2.695 | 19.036 | 1.00 | 40.19 |
| ATOM | 6290 | NE2 | HIS | 3201 | 6.867 | −3.500 | 18.121 | 1.00 | 40.73 |
| ATOM | 6291 | C | HIS | 3201 | 10.978 | 0.192 | 20.465 | 1.00 | 39.03 |
| ATOM | 6292 | O | HIS | 3201 | 11.534 | −0.475 | 21.333 | 1.00 | 38.04 |
| ATOM | 6293 | N | ARG | 3202 | 11.523 | 1.290 | 19.957 | 1.00 | 38.42 |
| ATOM | 6294 | CA | ARG | 3202 | 12.788 | 1.775 | 20.478 | 1.00 | 37.66 |
| ATOM | 6295 | CB | ARG | 3202 | 13.979 | 1.192 | 19.693 | 1.00 | 37.22 |
| ATOM | 6296 | CG | ARG | 3202 | 14.136 | 1.692 | 18.271 | 1.00 | 37.18 |
| ATOM | 6297 | CD | ARG | 3202 | 15.197 | 0.897 | 17.509 | 1.00 | 36.43 |
| ATOM | 6298 | NE | ARG | 3202 | 16.549 | 1.048 | 18.057 | 1.00 | 35.84 |
| ATOM | 6299 | CZ | ARG | 3202 | 17.309 | 2.138 | 17.929 | 1.00 | 34.55 |
| ATOM | 6300 | NH1 | ARG | 3202 | 16.867 | 3.201 | 17.265 | 1.00 | 33.05 |
| ATOM | 6301 | NH2 | ARG | 3202 | 18.520 | 2.162 | 18.469 | 1.00 | 32.83 |
| ATOM | 6302 | C | ARG | 3202 | 12.791 | 3.290 | 20.435 | 1.00 | 38.19 |
| ATOM | 6303 | O | ARG | 3202 | 12.181 | 3.903 | 19.558 | 1.00 | 38.31 |
| ATOM | 6304 | N | ILE | 3203 | 13.453 | 3.891 | 21.412 | 1.00 | 38.58 |
| ATOM | 6305 | CA | ILE | 3203 | 13.547 | 5.333 | 21.486 | 1.00 | 39.83 |
| ATOM | 6306 | CB | ILE | 3203 | 14.358 | 5.731 | 22.720 | 1.00 | 40.59 |
| ATOM | 6307 | CG2 | ILE | 3203 | 15.647 | 4.903 | 22.782 | 1.00 | 41.46 |
| ATOM | 6308 | CG1 | ILE | 3203 | 14.650 | 7.225 | 22.669 | 1.00 | 41.18 |
| ATOM | 6309 | CD1 | ILE | 3203 | 15.454 | 7.703 | 23.816 | 1.00 | 42.07 |
| ATOM | 6310 | C | ILE | 3203 | 14.237 | 5.850 | 20.218 | 1.00 | 40.01 |
| ATOM | 6311 | O | ILE | 3203 | 15.319 | 5.391 | 19.857 | 1.00 | 39.70 |
| ATOM | 6312 | N | GLY | 3204 | 13.609 | 6.805 | 19.544 | 1.00 | 40.57 |
| ATOM | 6313 | CA | GLY | 3204 | 14.187 | 7.325 | 18.317 | 1.00 | 40.51 |
| ATOM | 6314 | C | GLY | 3204 | 13.725 | 6.535 | 17.098 | 1.00 | 40.21 |
| ATOM | 6315 | O | GLY | 3204 | 13.913 | 6.958 | 15.956 | 1.00 | 39.93 |
| ATOM | 6316 | N | GLY | 3205 | 13.116 | 5.380 | 17.345 | 1.00 | 40.06 |
| ATOM | 6317 | CA | GLY | 3205 | 12.635 | 4.553 | 16.259 | 1.00 | 39.84 |
| ATOM | 6318 | C | GLY | 3205 | 13.723 | 4.129 | 15.296 | 1.00 | 39.75 |
| ATOM | 6319 | O | GLY | 3205 | 14.897 | 4.051 | 15.657 | 1.00 | 39.35 |
| ATOM | 6320 | N | TYR | 3206 | 13.320 | 3.860 | 14.058 | 1.00 | 39.43 |
| ATOM | 6321 | CA | TYR | 3206 | 14.240 | 3.429 | 13.022 | 1.00 | 39.47 |
| ATOM | 6322 | CB | TYR | 3206 | 14.310 | 1.895 | 13.023 | 1.00 | 39.17 |
| ATOM | 6323 | CG | TYR | 3206 | 12.997 | 1.209 | 12.697 | 1.00 | 39.15 |
| ATOM | 6324 | CD1 | TYR | 3206 | 12.440 | 1.303 | 11.419 | 1.00 | 39.17 |
| ATOM | 6325 | CE1 | TYR | 3206 | 11.226 | 0.707 | 11.113 | 1.00 | 39.59 |
| ATOM | 6326 | CD2 | TYR | 3206 | 12.298 | 0.486 | 13.670 | 1.00 | 39.58 |
| ATOM | 6327 | CE2 | TYR | 3206 | 11.065 | −0.124 | 13.372 | 1.00 | 39.98 |
| ATOM | 6328 | CZ | TYR | 3206 | 10.536 | −0.004 | 12.084 | 1.00 | 40.25 |
| ATOM | 6329 | OH | TYR | 3206 | 9.319 | −0.576 | 11.753 | 1.00 | 40.60 |
| ATOM | 6330 | C | TYR | 3206 | 13.753 | 3.937 | 11.667 | 1.00 | 39.99 |
| ATOM | 6331 | O | TYR | 3206 | 12.625 | 4.406 | 11.547 | 1.00 | 39.69 |
| ATOM | 6332 | N | LYS | 3207 | 14.595 | 3.846 | 10.646 | 1.00 | 40.79 |
| ATOM | 6333 | CA | LYS | 3207 | 14.190 | 4.293 | 9.327 | 1.00 | 42.17 |
| ATOM | 6334 | CB | LYS | 3207 | 14.907 | 5.593 | 8.953 | 1.00 | 43.85 |
| ATOM | 6335 | CG | LYS | 3207 | 14.332 | 6.762 | 9.745 | 1.00 | 46.38 |
| ATOM | 6336 | CD | LYS | 3207 | 14.807 | 8.122 | 9.287 | 1.00 | 48.03 |
| ATOM | 6337 | CE | LYS | 3207 | 13.873 | 9.190 | 9.850 | 1.00 | 48.88 |
| ATOM | 6338 | NZ | LYS | 3207 | 14.296 | 10.584 | 9.527 | 1.00 | 50.45 |
| ATOM | 6339 | C | LYS | 3207 | 14.383 | 3.229 | 8.268 | 1.00 | 42.26 |
| ATOM | 6340 | O | LYS | 3207 | 15.420 | 2.576 | 8.196 | 1.00 | 42.41 |
| ATOM | 6341 | N | VAL | 3208 | 13.347 | 3.044 | 7.462 | 1.00 | 42.60 |
| ATOM | 6342 | CA | VAL | 3208 | 13.368 | 2.056 | 6.404 | 1.00 | 42.75 |
| ATOM | 6343 | CB | VAL | 3208 | 12.111 | 1.171 | 6.467 | 1.00 | 41.86 |
| ATOM | 6344 | CG1 | VAL | 3208 | 12.039 | 0.270 | 5.251 | 1.00 | 42.36 |
| ATOM | 6345 | CG2 | VAL | 3208 | 12.137 | 0.344 | 7.728 | 1.00 | 42.06 |
| ATOM | 6346 | C | VAL | 3208 | 13.401 | 2.758 | 5.060 | 1.00 | 43.13 |
| ATOM | 6347 | O | VAL | 3208 | 12.434 | 3.403 | 4.682 | 1.00 | 43.20 |
| ATOM | 6348 | N | ARG | 3209 | 14.521 | 2.661 | 4.352 | 1.00 | 43.70 |
| ATOM | 6349 | CA | ARG | 3209 | 14.608 | 3.268 | 3.033 | 1.00 | 44.54 |
| ATOM | 6350 | CB | ARG | 3209 | 15.973 | 3.905 | 2.791 | 1.00 | 46.46 |
| ATOM | 6351 | CG | ARG | 3209 | 16.218 | 4.320 | 1.332 | 1.00 | 49.71 |
| ATOM | 6352 | CD | ARG | 3209 | 15.176 | 5.314 | 0.763 | 1.00 | 52.07 |
| ATOM | 6353 | NE | ARG | 3209 | 13.801 | 4.795 | 0.746 | 1.00 | 53.65 |
| ATOM | 6354 | CZ | ARG | 3209 | 12.835 | 5.238 | −0.062 | 1.00 | 53.61 |
| ATOM | 6355 | NH1 | ARG | 3209 | 13.087 | 6.206 | −0.936 | 1.00 | 53.36 |
| ATOM | 6356 | NH2 | ARG | 3209 | 11.612 | 4.728 | 0.018 | 1.00 | 52.96 |
| ATOM | 6357 | C | ARG | 3209 | 14.386 | 2.150 | 2.036 | 1.00 | 44.29 |
| ATOM | 6358 | O | ARG | 3209 | 15.274 | 1.326 | 1.797 | 1.00 | 44.72 |
| ATOM | 6359 | N | TYR | 3210 | 13.195 | 2.120 | 1.452 | 1.00 | 43.42 |
| ATOM | 6360 | CA | TYR | 3210 | 12.844 | 1.081 | 0.495 | 1.00 | 42.95 |
| ATOM | 6361 | CB | TYR | 3210 | 11.393 | 1.239 | 0.050 | 1.00 | 43.74 |
| ATOM | 6362 | CG | TYR | 3210 | 10.431 | 1.048 | 1.177 | 1.00 | 44.73 |
| ATOM | 6363 | CD1 | TYR | 3210 | 10.254 | 2.042 | 2.134 | 1.00 | 45.66 |
| ATOM | 6364 | CE1 | TYR | 3210 | 9.468 | 1.827 | 3.254 | 1.00 | 47.16 |
| ATOM | 6365 | CD2 | TYR | 3210 | 9.785 | −0.171 | 1.359 | 1.00 | 45.97 |
| ATOM | 6366 | CE2 | TYR | 3210 | 8.995 | −0.405 | 2.477 | 1.00 | 46.88 |
| ATOM | 6367 | CZ | TYR | 3210 | 8.846 | 0.595 | 3.423 | 1.00 | 47.53 |
| ATOM | 6368 | OH | TYR | 3210 | 8.113 | 0.349 | 4.564 | 1.00 | 49.31 |
| ATOM | 6369 | C | TYR | 3210 | 13.716 | 1.041 | −0.733 | 1.00 | 42.28 |
| ATOM | 6370 | O | TYR | 3210 | 13.968 | −0.027 | −1.286 | 1.00 | 42.12 |
| ATOM | 6371 | N | ALA | 3211 | 14.167 | 2.204 | −1.173 | 1.00 | 41.86 |
| ATOM | 6372 | CA | ALA | 3211 | 14.992 | 2.267 | −2.368 | 1.00 | 41.16 |
| ATOM | 6373 | CB | ALA | 3211 | 15.335 | 3.706 | −2.680 | 1.00 | 41.07 |
| ATOM | 6374 | C | ALA | 3211 | 16.260 | 1.448 | −2.205 | 1.00 | 40.97 |
| ATOM | 6375 | O | ALA | 3211 | 16.771 | 0.885 | −3.169 | 1.00 | 41.36 |
| ATOM | 6376 | N | THR | 3212 | 16.760 | 1.375 | −0.977 | 1.00 | 40.42 |
| ATOM | 6377 | CA | THR | 3212 | 17.986 | 0.637 | −0.711 | 1.00 | 39.72 |
| ATOM | 6378 | CB | THR | 3212 | 18.996 | 1.527 | 0.053 | 1.00 | 40.40 |
| ATOM | 6379 | OG1 | THR | 3212 | 18.385 | 2.033 | 1.251 | 1.00 | 40.29 |
| ATOM | 6380 | CG2 | THR | 3212 | 19.437 | 2.696 | −0.826 | 1.00 | 39.94 |
| ATOM | 6381 | C | THR | 3212 | 17.736 | −0.640 | 0.080 | 1.00 | 38.77 |
| ATOM | 6382 | O | THR | 3212 | 18.675 | −1.279 | 0.552 | 1.00 | 38.32 |
| ATOM | 6383 | N | TRP | 3213 | 16.467 | −1.004 | 0.223 | 1.00 | 38.07 |
| ATOM | 6384 | CA | TRP | 3213 | 16.095 | −2.208 | 0.955 | 1.00 | 37.69 |
| ATOM | 6385 | CB | TRP | 3213 | 16.415 | −3.445 | 0.113 | 1.00 | 38.71 |
| ATOM | 6386 | CG | TRP | 3213 | 15.801 | −3.410 | −1.260 | 1.00 | 39.88 |
| ATOM | 6387 | CD2 | TRP | 3213 | 14.547 | −3.981 | −1.644 | 1.00 | 39.78 |
| ATOM | 6388 | CE2 | TRP | 3213 | 14.338 | −3.659 | −3.004 | 1.00 | 39.97 |
| ATOM | 6389 | CE3 | TRP | 3213 | 13.576 | −4.733 | −0.968 | 1.00 | 39.38 |
| ATOM | 6390 | CD1 | TRP | 3213 | 16.294 | −2.780 | −2.379 | 1.00 | 39.94 |
| ATOM | 6391 | NE1 | TRP | 3213 | 15.418 | −2.926 | −3.429 | 1.00 | 39.80 |
| ATOM | 6392 | CZ2 | TRP | 3213 | 13.195 | −4.066 | −3.701 | 1.00 | 40.13 |
| ATOM | 6393 | CZ3 | TRP | 3213 | 12.441 | −5.137 | −1.660 | 1.00 | 39.88 |
| ATOM | 6394 | CH2 | TRP | 3213 | 12.260 | −4.803 | −3.014 | 1.00 | 39.80 |
| ATOM | 6395 | C | TRP | 3213 | 16.840 | −2.281 | 2.282 | 1.00 | 37.23 |
| ATOM | 6396 | O | TRP | 3213 | 17.426 | −3.308 | 2.627 | 1.00 | 36.36 |
| ATOM | 6397 | N | SER | 3214 | 16.812 | −1.186 | 3.030 | 1.00 | 36.58 |
| ATOM | 6398 | CA | SER | 3214 | 17.522 | −1.163 | 4.293 | 1.00 | 36.48 |
| ATOM | 6399 | CB | SER | 3214 | 18.835 | −0.406 | 4.124 | 1.00 | 36.91 |
| ATOM | 6400 | OG | SER | 3214 | 18.589 | 0.858 | 3.540 | 1.00 | 38.53 |
| ATOM | 6401 | C | SER | 3214 | 16.736 | −0.587 | 5.454 | 1.00 | 35.52 |
| ATOM | 6402 | O | SER | 3214 | 15.722 | 0.096 | 5.277 | 1.00 | 34.85 |
| ATOM | 6403 | N | ILE | 3215 | 17.215 | −0.913 | 6.647 | 1.00 | 34.36 |
| ATOM | 6404 | CA | ILE | 3215 | 16.636 | −0.443 | 7.888 | 1.00 | 33.91 |
| ATOM | 6405 | CB | ILE | 3215 | 16.002 | −1.599 | 8.670 | 1.00 | 33.92 |
| ATOM | 6406 | CG2 | ILE | 3215 | 17.015 | −2.680 | 8.920 | 1.00 | 34.47 |
| ATOM | 6407 | CG1 | ILE | 3215 | 15.427 | −1.077 | 9.979 | 1.00 | 34.07 |
| ATOM | 6408 | CD1 | ILE | 3215 | 14.706 | −2.131 | 10.780 | 1.00 | 34.38 |
| ATOM | 6409 | C | ILE | 3215 | 17.806 | 0.151 | 8.661 | 1.00 | 33.13 |
| ATOM | 6410 | O | ILE | 3215 | 18.872 | −0.454 | 8.742 | 1.00 | 32.83 |
| ATOM | 6411 | N | ILE | 3216 | 17.616 | 1.352 | 9.194 | 1.00 | 32.76 |
| ATOM | 6412 | CA | ILE | 3216 | 18.667 | 2.036 | 9.936 | 1.00 | 32.20 |
| ATOM | 6413 | CB | ILE | 3216 | 18.963 | 3.429 | 9.342 | 1.00 | 32.44 |
| ATOM | 6414 | CG2 | ILE | 3216 | 20.110 | 4.086 | 10.088 | 1.00 | 31.04 |
| ATOM | 6415 | CG1 | ILE | 3216 | 19.286 | 3.308 | 7.857 | 1.00 | 33.28 |
| ATOM | 6416 | CD1 | ILE | 3216 | 19.352 | 4.649 | 7.157 | 1.00 | 33.75 |
| ATOM | 6417 | C | ILE | 3216 | 18.257 | 2.265 | 11.377 | 1.00 | 31.96 |
| ATOM | 6418 | O | ILE | 3216 | 17.158 | 2.754 | 11.638 | 1.00 | 31.00 |
| ATOM | 6419 | N | MSE | 3217 | 19.138 | 1.903 | 12.306 | 1.00 | 31.79 |
| ATOM | 6420 | CA | MSE | 3217 | 18.880 | 2.136 | 13.716 | 1.00 | 31.79 |
| ATOM | 6421 | CB | MSE | 3217 | 18.840 | 0.820 | 14.508 | 1.00 | 31.42 |
| ATOM | 6422 | CG | MSE | 3217 | 17.603 | −0.064 | 14.255 | 1.00 | 31.42 |
| ATOM | 6423 | SE | MSE | 3217 | 17.504 | −1.556 | 15.341 | 1.00 | 31.78 |
| ATOM | 6424 | CE | MSE | 3217 | 18.604 | −2.673 | 14.447 | 1.00 | 30.54 |
| ATOM | 6425 | C | MSE | 3217 | 20.021 | 3.022 | 14.207 | 1.00 | 32.32 |
| ATOM | 6426 | O | MSE | 3217 | 21.197 | 2.661 | 14.114 | 1.00 | 32.43 |
| ATOM | 6427 | N | ASP | 3218 | 19.664 | 4.194 | 14.713 | 1.00 | 32.71 |
| ATOM | 6428 | CA | ASP | 3218 | 20.628 | 5.147 | 15.229 | 1.00 | 33.09 |
| ATOM | 6429 | CB | ASP | 3218 | 20.022 | 6.544 | 15.123 | 1.00 | 35.67 |
| ATOM | 6430 | CG | ASP | 3218 | 21.063 | 7.625 | 15.018 | 1.00 | 38.30 |
| ATOM | 6431 | OD1 | ASP | 3218 | 20.843 | 8.702 | 15.618 | 1.00 | 39.16 |
| ATOM | 6432 | OD2 | ASP | 3218 | 22.089 | 7.401 | 14.327 | 1.00 | 40.02 |
| ATOM | 6433 | C | ASP | 3218 | 20.927 | 4.813 | 16.696 | 1.00 | 32.45 |
| ATOM | 6434 | O | ASP | 3218 | 20.043 | 4.380 | 17.417 | 1.00 | 32.49 |
| ATOM | 6435 | N | SER | 3219 | 22.174 | 5.014 | 17.125 | 1.00 | 32.52 |
| ATOM | 6436 | CA | SER | 3219 | 22.611 | 4.753 | 18.505 | 1.00 | 31.30 |
| ATOM | 6437 | CB | SER | 3219 | 22.279 | 5.934 | 19.416 | 1.00 | 30.55 |
| ATOM | 6438 | OG | SER | 3219 | 22.987 | 7.080 | 19.009 | 1.00 | 31.34 |
| ATOM | 6439 | C | SER | 3219 | 22.046 | 3.496 | 19.143 | 1.00 | 30.80 |
| ATOM | 6440 | O | SER | 3219 | 21.242 | 3.578 | 20.073 | 1.00 | 31.63 |
| ATOM | 6441 | N | VAL | 3220 | 22.473 | 2.335 | 18.663 | 1.00 | 29.85 |
| ATOM | 6442 | CA | VAL | 3220 | 21.990 | 1.084 | 19.223 | 1.00 | 29.07 |
| ATOM | 6443 | CB | VAL | 3220 | 22.536 | −0.142 | 18.423 | 1.00 | 29.06 |
| ATOM | 6444 | CG1 | VAL | 3220 | 22.155 | −0.004 | 16.960 | 1.00 | 28.03 |
| ATOM | 6445 | CG2 | VAL | 3220 | 24.050 | −0.255 | 18.571 | 1.00 | 28.64 |
| ATOM | 6446 | C | VAL | 3220 | 22.375 | 0.966 | 20.696 | 1.00 | 28.95 |
| ATOM | 6447 | O | VAL | 3220 | 23.376 | 1.526 | 21.136 | 1.00 | 29.63 |
| ATOM | 6448 | N | VAL | 3221 | 21.562 | 0.236 | 21.447 | 1.00 | 28.67 |
| ATOM | 6449 | CA | VAL | 3221 | 21.777 | 0.032 | 22.868 | 1.00 | 28.31 |
| ATOM | 6450 | CB | VAL | 3221 | 20.786 | 0.926 | 23.649 | 1.00 | 29.03 |
| ATOM | 6451 | CG1 | VAL | 3221 | 21.003 | 2.391 | 23.252 | 1.00 | 27.53 |
| ATOM | 6452 | CG2 | VAL | 3221 | 19.332 | 0.524 | 23.321 | 1.00 | 27.80 |
| ATOM | 6453 | C | VAL | 3221 | 21.545 | −1.456 | 23.157 | 1.00 | 28.57 |
| ATOM | 6454 | O | VAL | 3221 | 20.952 | −2.171 | 22.343 | 1.00 | 29.31 |
| ATOM | 6455 | N | PRO | 3222 | 21.989 | −1.940 | 24.323 | 1.00 | 28.46 |
| ATOM | 6456 | CD | PRO | 3222 | 22.521 | −1.169 | 25.462 | 1.00 | 28.30 |
| ATOM | 6457 | CA | PRO | 3222 | 21.817 | −3.356 | 24.670 | 1.00 | 28.18 |
| ATOM | 6458 | CB | PRO | 3222 | 22.143 | −3.383 | 26.161 | 1.00 | 28.32 |
| ATOM | 6459 | CG | PRO | 3222 | 23.155 | −2.251 | 26.308 | 1.00 | 28.56 |
| ATOM | 6460 | C | PRO | 3222 | 20.442 | −3.944 | 24.353 | 1.00 | 28.22 |
| ATOM | 6461 | O | PRO | 3222 | 20.349 | −5.090 | 23.907 | 1.00 | 29.25 |
| ATOM | 6462 | N | SER | 3223 | 19.379 | −3.173 | 24.566 | 1.00 | 28.05 |
| ATOM | 6463 | CA | SER | 3223 | 18.037 | −3.695 | 24.304 | 1.00 | 28.14 |
| ATOM | 6464 | CB | SER | 3223 | 16.969 | −2.830 | 24.990 | 1.00 | 26.52 |
| ATOM | 6465 | OG | SER | 3223 | 17.035 | −1.482 | 24.585 | 1.00 | 24.97 |
| ATOM | 6466 | C | SER | 3223 | 17.743 | −3.862 | 22.811 | 1.00 | 28.85 |
| ATOM | 6467 | O | SER | 3223 | 16.705 | −4.400 | 22.429 | 1.00 | 29.59 |
| ATOM | 6468 | N | ASP | 3224 | 18.668 | −3.430 | 21.964 | 1.00 | 29.32 |
| ATOM | 6469 | CA | ASP | 3224 | 18.474 | −3.588 | 20.534 | 1.00 | 30.70 |
| ATOM | 6470 | CB | ASP | 3224 | 19.201 | −2.478 | 19.748 | 1.00 | 30.81 |
| ATOM | 6471 | CG | ASP | 3224 | 18.518 | −1.114 | 19.867 | 1.00 | 31.28 |
| ATOM | 6472 | OD1 | ASP | 3224 | 17.272 | −1.052 | 19.900 | 1.00 | 31.54 |
| ATOM | 6473 | OD2 | ASP | 3224 | 19.231 | −0.093 | 19.911 | 1.00 | 32.22 |
| ATOM | 6474 | C | ASP | 3224 | 19.002 | −4.959 | 20.107 | 1.00 | 31.30 |
| ATOM | 6475 | O | ASP | 3224 | 18.774 | −5.399 | 18.974 | 1.00 | 31.98 |
| ATOM | 6476 | N | LYS | 3225 | 19.712 | −5.629 | 21.012 | 1.00 | 31.00 |
| ATOM | 6477 | CA | LYS | 3225 | 20.258 | −6.952 | 20.721 | 1.00 | 31.82 |
| ATOM | 6478 | CB | LYS | 3225 | 20.870 | −7.565 | 21.979 | 1.00 | 33.14 |
| ATOM | 6479 | CG | LYS | 3225 | 22.146 | −6.921 | 22.511 | 1.00 | 35.17 |
| ATOM | 6480 | CD | LYS | 3225 | 22.353 | −7.411 | 23.938 | 1.00 | 36.39 |
| ATOM | 6481 | CE | LYS | 3225 | 23.803 | −7.394 | 24.348 | 1.00 | 38.28 |
| ATOM | 6482 | NZ | LYS | 3225 | 24.048 | −8.429 | 25.400 | 1.00 | 39.21 |
| ATOM | 6483 | C | LYS | 3225 | 19.167 | −7.903 | 20.226 | 1.00 | 31.85 |
| ATOM | 6484 | O | LYS | 3225 | 18.058 | −7.924 | 20.763 | 1.00 | 31.37 |
| ATOM | 6485 | N | GLY | 3226 | 19.485 | −8.700 | 19.213 | 1.00 | 31.83 |
| ATOM | 6486 | CA | GLY | 3226 | 18.511 | −9.645 | 18.706 | 1.00 | 32.42 |
| ATOM | 6487 | C | GLY | 3226 | 18.677 | −10.012 | 17.245 | 1.00 | 32.68 |
| ATOM | 6488 | O | GLY | 3226 | 19.642 | −9.617 | 16.592 | 1.00 | 33.42 |
| ATOM | 6489 | N | ASN | 3227 | 17.723 | −10.782 | 16.739 | 1.00 | 32.39 |
| ATOM | 6490 | CA | ASN | 3227 | 17.726 | −11.218 | 15.359 | 1.00 | 32.70 |
| ATOM | 6491 | CB | ASN | 3227 | 17.202 | −12.642 | 15.249 | 1.00 | 32.74 |
| ATOM | 6492 | CG | ASN | 3227 | 18.197 | −13.660 | 15.700 | 1.00 | 33.48 |
| ATOM | 6493 | OD1 | ASN | 3227 | 19.352 | −13.646 | 15.281 | 1.00 | 33.92 |
| ATOM | 6494 | ND2 | ASN | 3227 | 17.755 | −14.574 | 16.552 | 1.00 | 34.71 |
| ATOM | 6495 | C | ASN | 3227 | 16.810 | −10.325 | 14.554 | 1.00 | 33.33 |
| ATOM | 6496 | O | ASN | 3227 | 15.666 | −10.080 | 14.947 | 1.00 | 34.14 |
| ATOM | 6497 | N | TYR | 3228 | 17.298 | −9.846 | 13.420 | 1.00 | 33.10 |
| ATOM | 6498 | CA | TYR | 3228 | 16.485 | −8.997 | 12.574 | 1.00 | 33.07 |
| ATOM | 6499 | CB | TYR | 3228 | 17.151 | −7.632 | 12.415 | 1.00 | 31.91 |
| ATOM | 6500 | CG | TYR | 3228 | 17.150 | −6.855 | 13.710 | 1.00 | 31.75 |
| ATOM | 6501 | CD1 | TYR | 3228 | 18.032 | −7.177 | 14.751 | 1.00 | 30.54 |
| ATOM | 6502 | CE1 | TYR | 3228 | 17.987 | −6.489 | 15.965 | 1.00 | 30.09 |
| ATOM | 6503 | CD2 | TYR | 3228 | 16.226 | −5.829 | 13.920 | 1.00 | 30.93 |
| ATOM | 6504 | CE2 | TYR | 3228 | 16.174 | −5.145 | 15.119 | 1.00 | 30.76 |
| ATOM | 6505 | CZ | TYR | 3228 | 17.056 | −5.472 | 16.140 | 1.00 | 30.04 |
| ATOM | 6506 | OH | TYR | 3228 | 17.004 | −4.750 | 17.308 | 1.00 | 29.57 |
| ATOM | 6507 | C | TYR | 3228 | 16.308 | −9.694 | 11.243 | 1.00 | 33.69 |
| ATOM | 6508 | O | TYR | 3228 | 17.281 | −10.017 | 10.559 | 1.00 | 33.68 |
| ATOM | 6509 | N | THR | 3229 | 15.051 | −9.940 | 10.897 | 1.00 | 34.87 |
| ATOM | 6510 | CA | THR | 3229 | 14.706 | −10.631 | 9.666 | 1.00 | 35.84 |
| ATOM | 6511 | CB | THR | 3229 | 13.782 | −11.814 | 9.947 | 1.00 | 35.85 |
| ATOM | 6512 | OG1 | THR | 3229 | 14.371 | −12.656 | 10.942 | 1.00 | 36.71 |
| ATOM | 6513 | CG2 | THR | 3229 | 13.541 | −12.601 | 8.688 | 1.00 | 34.59 |
| ATOM | 6514 | C | THR | 3229 | 13.969 | −9.731 | 8.702 | 1.00 | 36.90 |
| ATOM | 6515 | O | THR | 3229 | 13.017 | −9.037 | 9.086 | 1.00 | 37.21 |
| ATOM | 6516 | N | CYS | 3230 | 14.403 | −9.740 | 7.449 | 1.00 | 37.26 |
| ATOM | 6517 | CA | CYS | 3230 | 13.723 | −8.938 | 6.452 | 1.00 | 38.82 |
| ATOM | 6518 | CB | CYS | 3230 | 14.708 | −8.190 | 5.581 | 1.00 | 38.93 |
| ATOM | 6519 | SG | CYS | 3230 | 15.459 | −9.286 | 4.434 | 1.00 | 42.17 |
| ATOM | 6520 | C | CYS | 3230 | 12.932 | −9.923 | 5.610 | 1.00 | 39.18 |
| ATOM | 6521 | O | CYS | 3230 | 13.396 | −11.026 | 5.327 | 1.00 | 39.13 |
| ATOM | 6522 | N | ILE | 3231 | 11.722 | −9.530 | 5.242 | 1.00 | 40.09 |
| ATOM | 6523 | CA | ILE | 3231 | 10.865 | −10.382 | 4.441 | 1.00 | 40.96 |
| ATOM | 6524 | CB | ILE | 3231 | 9.624 | −10.821 | 5.239 | 1.00 | 41.31 |
| ATOM | 6525 | CG2 | ILE | 3231 | 8.651 | −11.586 | 4.336 | 1.00 | 40.65 |
| ATOM | 6526 | CG1 | ILE | 3231 | 10.075 | −11.701 | 6.412 | 1.00 | 41.15 |
| ATOM | 6527 | CD1 | ILE | 3231 | 8.947 | −12.218 | 7.271 | 1.00 | 41.25 |
| ATOM | 6528 | C | ILE | 3231 | 10.457 | −9.655 | 3.174 | 1.00 | 42.07 |
| ATOM | 6529 | O | ILE | 3231 | 9.714 | −8.670 | 3.210 | 1.00 | 42.06 |
| ATOM | 6530 | N | VAL | 3232 | 10.985 | −10.142 | 2.057 | 1.00 | 43.27 |
| ATOM | 6531 | CA | VAL | 3232 | 10.723 | −9.573 | 0.740 | 1.00 | 45.00 |
| ATOM | 6532 | CB | VAL | 3232 | 12.035 | −9.474 | −0.075 | 1.00 | 44.47 |
| ATOM | 6533 | CG1 | VAL | 3232 | 11.764 | −8.896 | −1.446 | 1.00 | 43.83 |
| ATOM | 6534 | CG2 | VAL | 3232 | 13.042 | −8.620 | 0.676 | 1.00 | 44.17 |
| ATOM | 6535 | C | VAL | 3232 | 9.728 | −10.472 | 0.007 | 1.00 | 46.56 |
| ATOM | 6536 | O | VAL | 3232 | 9.997 | −11.650 | −0.234 | 1.00 | 46.12 |
| ATOM | 6537 | N | GLU | 3233 | 8.583 | −9.915 | −0.362 | 1.00 | 48.54 |
| ATOM | 6538 | CA | GLU | 3233 | 7.565 | −10.717 | −1.024 | 1.00 | 50.43 |
| ATOM | 6539 | CB | GLU | 3233 | 6.677 | −11.359 | 0.041 | 1.00 | 51.98 |
| ATOM | 6540 | CG | GLU | 3233 | 6.161 | −10.326 | 1.042 | 1.00 | 55.35 |
| ATOM | 6541 | CD | GLU | 3233 | 5.295 | −10.912 | 2.149 | 1.00 | 57.38 |
| ATOM | 6542 | OE1 | GLU | 3233 | 5.142 | −10.233 | 3.200 | 1.00 | 58.04 |
| ATOM | 6543 | OE2 | GLU | 3233 | 4.764 | −12.035 | 1.967 | 1.00 | 58.23 |
| ATOM | 6544 | C | GLU | 3233 | 6.671 | −9.976 | −2.007 | 1.00 | 50.42 |
| ATOM | 6545 | O | GLU | 3233 | 6.478 | −8.765 | −1.919 | 1.00 | 49.95 |
| ATOM | 6546 | N | ASN | 3234 | 6.134 | −10.746 | −2.946 | 1.00 | 51.30 |
| ATOM | 6547 | CA | ASN | 3234 | 5.195 | −10.265 | −3.950 | 1.00 | 52.16 |
| ATOM | 6548 | CB | ASN | 3234 | 5.904 | −9.869 | −5.253 | 1.00 | 51.61 |
| ATOM | 6549 | CG | ASN | 3234 | 6.478 | −11.052 | −6.005 | 1.00 | 51.46 |
| ATOM | 6550 | OD1 | ASN | 3234 | 6.178 | −12.207 | −5.706 | 1.00 | 50.91 |
| ATOM | 6551 | ND2 | ASN | 3234 | 7.304 | −10.764 | −7.008 | 1.00 | 51.17 |
| ATOM | 6552 | C | ASN | 3234 | 4.234 | −11.432 | −4.189 | 1.00 | 53.24 |
| ATOM | 6553 | O | ASN | 3234 | 4.408 | −12.514 | −3.614 | 1.00 | 53.02 |
| ATOM | 6554 | N | GLU | 3235 | 3.232 | −11.224 | −5.034 | 1.00 | 54.33 |
| ATOM | 6555 | CA | GLU | 3235 | 2.248 | −12.264 | −5.309 | 1.00 | 55.01 |
| ATOM | 6556 | CB | GLU | 3235 | 1.263 | −11.762 | −6.363 | 1.00 | 56.23 |
| ATOM | 6557 | CG | GLU | 3235 | −0.056 | −12.502 | −6.368 | 1.00 | 59.08 |
| ATOM | 6558 | CD | GLU | 3235 | −1.109 | −11.820 | −7.236 | 1.00 | 61.60 |
| ATOM | 6559 | OE1 | GLU | 3235 | −0.815 | −11.540 | −8.426 | 1.00 | 62.64 |
| ATOM | 6560 | OE2 | GLU | 3235 | −2.231 | −11.566 | −6.727 | 1.00 | 62.11 |
| ATOM | 6561 | C | GLU | 3235 | 2.844 | −13.611 | −5.736 | 1.00 | 54.64 |
| ATOM | 6562 | O | GLU | 3235 | 2.232 | −14.655 | −5.519 | 1.00 | 54.61 |
| ATOM | 6563 | N | TYR | 3236 | 4.047 | −13.595 | −6.303 | 1.00 | 54.33 |
| ATOM | 6564 | CA | TYR | 3236 | 4.679 | −14.826 | −6.776 | 1.00 | 53.98 |
| ATOM | 6565 | CB | TYR | 3236 | 5.366 | −14.577 | −8.125 | 1.00 | 53.88 |
| ATOM | 6566 | CG | TYR | 3236 | 4.434 | −14.113 | −9.222 | 1.00 | 54.02 |
| ATOM | 6567 | CD1 | TYR | 3236 | 3.836 | −12.854 | −9.169 | 1.00 | 54.22 |
| ATOM | 6568 | CE1 | TYR | 3236 | 2.954 | −12.428 | −10.167 | 1.00 | 54.42 |
| ATOM | 6569 | CD2 | TYR | 3236 | 4.130 | −14.940 | −10.304 | 1.00 | 54.27 |
| ATOM | 6570 | CE2 | TYR | 3236 | 3.249 | −14.523 | −11.309 | 1.00 | 54.13 |
| ATOM | 6571 | CZ | TYR | 3236 | 2.666 | −13.268 | −11.232 | 1.00 | 54.13 |
| ATOM | 6572 | OH | TYR | 3236 | 1.796 | −12.853 | −12.211 | 1.00 | 53.50 |
| ATOM | 6573 | C | TYR | 3236 | 5.672 | −15.516 | −5.845 | 1.00 | 54.06 |
| ATOM | 6574 | O | TYR | 3236 | 6.196 | −16.581 | −6.183 | 1.00 | 54.71 |
| ATOM | 6575 | N | GLY | 3237 | 5.945 | −14.933 | −4.684 | 1.00 | 53.27 |
| ATOM | 6576 | CA | GLY | 3237 | 6.884 | −15.576 | −3.782 | 1.00 | 51.95 |
| ATOM | 6577 | C | GLY | 3237 | 7.440 | −14.678 | −2.700 | 1.00 | 51.10 |
| ATOM | 6578 | O | GLY | 3237 | 7.245 | −13.457 | −2.720 | 1.00 | 50.97 |
| ATOM | 6579 | N | SER | 3238 | 8.129 | −15.287 | −1.741 | 1.00 | 49.86 |
| ATOM | 6580 | CA | SER | 3238 | 8.728 | −14.529 | −0.655 | 1.00 | 48.69 |
| ATOM | 6581 | CB | SER | 3238 | 7.781 | −14.430 | 0.542 | 1.00 | 49.49 |
| ATOM | 6582 | OG | SER | 3238 | 7.695 | −15.677 | 1.206 | 1.00 | 51.08 |
| ATOM | 6583 | C | SER | 3238 | 10.038 | −15.140 | −0.199 | 1.00 | 47.32 |
| ATOM | 6584 | O | SER | 3238 | 10.171 | −16.362 | −0.090 | 1.00 | 47.51 |
| ATOM | 6585 | N | ILE | 3239 | 11.008 | −14.266 | 0.047 | 1.00 | 45.32 |
| ATOM | 6586 | CA | ILE | 3239 | 12.317 | −14.668 | 0.525 | 1.00 | 42.89 |
| ATOM | 6587 | CB | ILE | 3239 | 13.422 | −14.331 | −0.491 | 1.00 | 41.39 |
| ATOM | 6588 | CG2 | ILE | 3239 | 13.409 | −15.345 | −1.623 | 1.00 | 40.63 |
| ATOM | 6589 | CG1 | ILE | 3239 | 13.241 | −12.903 | −1.006 | 1.00 | 39.72 |
| ATOM | 6590 | CD1 | ILE | 3239 | 14.288 | −12.478 | −1.999 | 1.00 | 37.79 |
| ATOM | 6591 | C | ILE | 3239 | 12.591 | −13.928 | 1.830 | 1.00 | 42.78 |
| ATOM | 6592 | O | ILE | 3239 | 11.852 | −13.013 | 2.205 | 1.00 | 41.85 |
| ATOM | 6593 | N | ASN | 3240 | 13.644 | −14.333 | 2.528 | 1.00 | 42.42 |
| ATOM | 6594 | CA | ASN | 3240 | 13.997 | −13.694 | 3.783 | 1.00 | 42.52 |
| ATOM | 6595 | CB | ASN | 3240 | 13.049 | −14.147 | 4.894 | 1.00 | 42.77 |
| ATOM | 6596 | CG | ASN | 3240 | 13.146 | −15.631 | 5.179 | 1.00 | 43.44 |
| ATOM | 6597 | OD1 | ASN | 3240 | 14.125 | −16.106 | 5.751 | 1.00 | 43.29 |
| ATOM | 6598 | ND2 | ASN | 3240 | 12.122 | −16.376 | 4.774 | 1.00 | 44.87 |
| ATOM | 6599 | C | ASN | 3240 | 15.429 | −13.973 | 4.195 | 1.00 | 42.38 |
| ATOM | 6600 | O | ASN | 3240 | 16.064 | −14.905 | 3.710 | 1.00 | 42.45 |
| ATOM | 6601 | N | HIS | 3241 | 15.933 | −13.147 | 5.099 | 1.00 | 41.92 |
| ATOM | 6602 | CA | HIS | 3241 | 17.289 | −13.296 | 5.594 | 1.00 | 41.59 |
| ATOM | 6603 | CB | HIS | 3241 | 18.266 | −12.509 | 4.720 | 1.00 | 42.38 |
| ATOM | 6604 | CG | HIS | 3241 | 19.704 | −12.737 | 5.065 | 1.00 | 43.96 |
| ATOM | 6605 | CD2 | HIS | 3241 | 20.623 | −11.925 | 5.643 | 1.00 | 44.63 |
| ATOM | 6606 | ND1 | HIS | 3241 | 20.350 | −13.929 | 4.816 | 1.00 | 44.28 |
| ATOM | 6607 | CE1 | HIS | 3241 | 21.604 | −13.842 | 5.223 | 1.00 | 44.58 |
| ATOM | 6608 | NE2 | HIS | 3241 | 21.796 | −12.637 | 5.730 | 1.00 | 45.28 |
| ATOM | 6609 | C | HIS | 3241 | 17.289 | −12.747 | 7.010 | 1.00 | 41.03 |
| ATOM | 6610 | O | HIS | 3241 | 16.503 | −11.847 | 7.335 | 1.00 | 41.15 |
| ATOM | 6611 | N | THR | 3242 | 18.157 | −13.284 | 7.857 | 1.00 | 39.70 |
| ATOM | 6612 | CA | THR | 3242 | 18.211 | −12.825 | 9.231 | 1.00 | 38.20 |
| ATOM | 6613 | CB | THR | 3242 | 17.636 | −13.883 | 10.178 | 1.00 | 38.08 |
| ATOM | 6614 | OG1 | THR | 3242 | 16.216 | −13.937 | 10.004 | 1.00 | 38.24 |
| ATOM | 6615 | CG2 | THR | 3242 | 17.945 | −13.542 | 11.628 | 1.00 | 38.20 |
| ATOM | 6616 | C | THR | 3242 | 19.603 | −12.457 | 9.680 | 1.00 | 37.60 |
| ATOM | 6617 | O | THR | 3242 | 20.553 | −13.211 | 9.482 | 1.00 | 37.52 |
| ATOM | 6618 | N | TYR | 3243 | 19.719 | −11.275 | 10.274 | 1.00 | 36.75 |
| ATOM | 6619 | CA | TYR | 3243 | 21.001 | −10.806 | 10.785 | 1.00 | 36.59 |
| ATOM | 6620 | CB | TYR | 3243 | 21.287 | −9.364 | 10.350 | 1.00 | 36.71 |
| ATOM | 6621 | CG | TYR | 3243 | 21.472 | −9.163 | 8.871 | 1.00 | 37.81 |
| ATOM | 6622 | CD1 | TYR | 3243 | 20.413 | −8.753 | 8.059 | 1.00 | 38.45 |
| ATOM | 6623 | CE1 | TYR | 3243 | 20.594 | −8.551 | 6.689 | 1.00 | 39.09 |
| ATOM | 6624 | CD2 | TYR | 3243 | 22.715 | −9.366 | 8.277 | 1.00 | 38.84 |
| ATOM | 6625 | CE2 | TYR | 3243 | 22.908 | −9.167 | 6.914 | 1.00 | 38.99 |
| ATOM | 6626 | CZ | TYR | 3243 | 21.849 | −8.760 | 6.127 | 1.00 | 39.51 |
| ATOM | 6627 | OH | TYR | 3243 | 22.057 | −8.561 | 4.781 | 1.00 | 40.19 |
| ATOM | 6628 | C | TYR | 3243 | 20.948 | −10.843 | 12.307 | 1.00 | 36.00 |
| ATOM | 6629 | O | TYR | 3243 | 19.876 | −10.712 | 12.908 | 1.00 | 35.18 |
| ATOM | 6630 | N | GLN | 3244 | 22.096 | −11.044 | 12.936 | 1.00 | 36.03 |
| ATOM | 6631 | CA | GLN | 3244 | 22.119 | −11.035 | 14.388 | 1.00 | 36.34 |
| ATOM | 6632 | CB | GLN | 3244 | 22.862 | −12.240 | 14.966 | 1.00 | 37.58 |
| ATOM | 6633 | CG | GLN | 3244 | 22.184 | −12.777 | 16.237 | 1.00 | 40.73 |
| ATOM | 6634 | CD | GLN | 3244 | 23.152 | −13.339 | 17.278 | 1.00 | 41.62 |
| ATOM | 6635 | OE1 | GLN | 3244 | 24.039 | −14.134 | 16.957 | 1.00 | 41.89 |
| ATOM | 6636 | NE2 | GLN | 3244 | 22.970 | −12.932 | 18.535 | 1.00 | 41.98 |
| ATOM | 6637 | C | GLN | 3244 | 22.826 | −9.764 | 14.817 | 1.00 | 35.47 |
| ATOM | 6638 | O | GLN | 3244 | 23.921 | −9.451 | 14.324 | 1.00 | 34.99 |
| ATOM | 6639 | N | LEU | 3245 | 22.186 | −9.014 | 15.708 | 1.00 | 34.40 |
| ATOM | 6640 | CA | LEU | 3245 | 22.788 | −7.790 | 16.205 | 1.00 | 33.17 |
| ATOM | 6641 | CB | LEU | 3245 | 21.823 | −6.609 | 16.105 | 1.00 | 32.28 |
| ATOM | 6642 | CG | LEU | 3245 | 22.317 | −5.331 | 16.806 | 1.00 | 30.60 |
| ATOM | 6643 | CD1 | LEU | 3245 | 23.708 | −4.973 | 16.343 | 1.00 | 28.60 |
| ATOM | 6644 | CD2 | LEU | 3245 | 21.351 | −4.201 | 16.532 | 1.00 | 29.97 |
| ATOM | 6645 | C | LEU | 3245 | 23.220 | −7.966 | 17.646 | 1.00 | 33.00 |
| ATOM | 6646 | O | LEU | 3245 | 22.432 | −8.353 | 18.507 | 1.00 | 32.93 |
| ATOM | 6647 | N | ASP | 3246 | 24.489 | −7.676 | 17.890 | 1.00 | 32.97 |
| ATOM | 6648 | CA | ASP | 3246 | 25.057 | −7.773 | 19.212 | 1.00 | 32.59 |
| ATOM | 6649 | CB | ASP | 3246 | 26.096 | −8.880 | 19.215 | 1.00 | 32.71 |
| ATOM | 6650 | CG | ASP | 3246 | 26.745 | −9.053 | 20.547 | 1.00 | 33.85 |
| ATOM | 6651 | OD1 | ASP | 3246 | 27.729 | −9.818 | 20.608 | 1.00 | 35.79 |
| ATOM | 6652 | OD2 | ASP | 3246 | 26.283 | −8.435 | 21.531 | 1.00 | 33.91 |
| ATOM | 6653 | C | ASP | 3246 | 25.692 | −6.420 | 19.565 | 1.00 | 32.97 |
| ATOM | 6654 | O | ASP | 3246 | 26.531 | −5.896 | 18.825 | 1.00 | 32.97 |
| ATOM | 6655 | N | VAL | 3247 | 25.273 | −5.853 | 20.692 | 1.00 | 32.99 |
| ATOM | 6656 | CA | VAL | 3247 | 25.784 | −4.572 | 21.156 | 1.00 | 32.92 |
| ATOM | 6657 | CB | VAL | 3247 | 24.627 | −3.637 | 21.551 | 1.00 | 33.23 |
| ATOM | 6658 | CG1 | VAL | 3247 | 25.172 | −2.328 | 22.105 | 1.00 | 33.01 |
| ATOM | 6659 | CG2 | VAL | 3247 | 23.735 | −3.384 | 20.349 | 1.00 | 33.02 |
| ATOM | 6660 | C | VAL | 3247 | 26.653 | −4.806 | 22.375 | 1.00 | 33.25 |
| ATOM | 6661 | O | VAL | 3247 | 26.208 | −5.412 | 23.344 | 1.00 | 33.29 |
| ATOM | 6662 | N | VAL | 3248 | 27.888 | −4.315 | 22.331 | 1.00 | 34.03 |
| ATOM | 6663 | CA | VAL | 3248 | 28.823 | −4.501 | 23.432 | 1.00 | 34.72 |
| ATOM | 6664 | CB | VAL | 3248 | 30.162 | −5.086 | 22.924 | 1.00 | 35.28 |
| ATOM | 6665 | CG1 | VAL | 3248 | 30.985 | −5.607 | 24.084 | 1.00 | 35.94 |
| ATOM | 6666 | CG2 | VAL | 3248 | 29.905 | −6.181 | 21.916 | 1.00 | 36.31 |
| ATOM | 6667 | C | VAL | 3248 | 29.128 | −3.217 | 24.184 | 1.00 | 36.05 |
| ATOM | 6668 | O | VAL | 3248 | 29.472 | −2.182 | 23.589 | 1.00 | 37.36 |
| ATOM | 6669 | N | GLU | 3249 | 29.001 | −3.308 | 25.502 | 1.00 | 36.96 |
| ATOM | 6670 | CA | GLU | 3249 | 29.289 | −2.187 | 26.379 | 1.00 | 38.35 |
| ATOM | 6671 | CB | GLU | 3249 | 28.322 | −2.162 | 27.554 | 1.00 | 39.73 |
| ATOM | 6672 | CG | GLU | 3249 | 26.936 | −1.720 | 27.193 | 1.00 | 42.89 |
| ATOM | 6673 | CD | GLU | 3249 | 26.049 | −1.596 | 28.413 | 1.00 | 45.25 |
| ATOM | 6674 | OE1 | GLU | 3249 | 25.453 | −2.623 | 28.836 | 1.00 | 46.40 |
| ATOM | 6675 | OE2 | GLU | 3249 | 25.966 | −0.466 | 28.952 | 1.00 | 45.81 |
| ATOM | 6676 | C | GLU | 3249 | 30.697 | −2.365 | 26.914 | 1.00 | 37.90 |
| ATOM | 6677 | O | GLU | 3249 | 31.119 | −3.490 | 27.203 | 1.00 | 38.59 |
| ATOM | 6678 | N | ARG | 3250 | 31.421 | −1.260 | 27.044 | 1.00 | 36.69 |
| ATOM | 6679 | CA | ARG | 3250 | 32.775 | −1.309 | 27.566 | 1.00 | 35.95 |
| ATOM | 6680 | CB | ARG | 3250 | 33.735 | −0.613 | 26.594 | 1.00 | 35.52 |
| ATOM | 6681 | CG | ARG | 3250 | 33.617 | −1.078 | 25.141 | 1.00 | 35.29 |
| ATOM | 6682 | CD | ARG | 3250 | 33.538 | −2.593 | 25.015 | 1.00 | 34.23 |
| ATOM | 6683 | NE | ARG | 3250 | 34.751 | −3.261 | 25.465 | 1.00 | 34.23 |
| ATOM | 6684 | CZ | ARG | 3250 | 35.884 | −3.320 | 24.772 | 1.00 | 34.05 |
| ATOM | 6685 | NH1 | ARG | 3250 | 35.969 | −2.751 | 23.578 | 1.00 | 33.69 |
| ATOM | 6686 | NH2 | ARG | 3250 | 36.936 | −3.952 | 25.279 | 1.00 | 33.15 |
| ATOM | 6687 | C | ARG | 3250 | 32.846 | −0.647 | 28.952 | 1.00 | 35.63 |
| ATOM | 6688 | O | ARG | 3250 | 31.973 | 0.140 | 29.320 | 1.00 | 35.45 |
| ATOM | 6689 | N | SER | 3251 | 33.869 | −1.001 | 29.726 | 1.00 | 35.32 |
| ATOM | 6690 | CA | SER | 3251 | 34.083 | −0.434 | 31.054 | 1.00 | 34.98 |
| ATOM | 6691 | CB | SER | 3251 | 34.042 | −1.521 | 32.128 | 1.00 | 34.57 |
| ATOM | 6692 | OG | SER | 3251 | 32.789 | −2.183 | 32.126 | 1.00 | 34.88 |
| ATOM | 6693 | C | SER | 3251 | 35.463 | 0.195 | 31.006 | 1.00 | 35.02 |
| ATOM | 6694 | O | SER | 3251 | 36.435 | −0.353 | 31.520 | 1.00 | 35.27 |
| ATOM | 6695 | N | PRO | 3252 | 35.567 | 1.356 | 30.360 | 1.00 | 34.81 |
| ATOM | 6696 | CD | PRO | 3252 | 34.491 | 2.166 | 29.770 | 1.00 | 34.70 |
| ATOM | 6697 | CA | PRO | 3252 | 36.844 | 2.042 | 30.250 | 1.00 | 34.80 |
| ATOM | 6698 | CB | PRO | 3252 | 36.551 | 3.135 | 29.240 | 1.00 | 34.74 |
| ATOM | 6699 | CG | PRO | 3252 | 35.164 | 3.509 | 29.599 | 1.00 | 34.75 |
| ATOM | 6700 | C | PRO | 3252 | 37.268 | 2.594 | 31.588 | 1.00 | 35.12 |
| ATOM | 6701 | O | PRO | 3252 | 37.346 | 3.808 | 31.775 | 1.00 | 36.00 |
| ATOM | 6702 | N | HIS | 3253 | 37.520 | 1.703 | 32.534 | 1.00 | 35.07 |
| ATOM | 6703 | CA | HIS | 3253 | 37.964 | 2.153 | 33.834 | 1.00 | 34.46 |
| ATOM | 6704 | CB | HIS | 3253 | 36.783 | 2.304 | 34.801 | 1.00 | 36.56 |
| ATOM | 6705 | CG | HIS | 3253 | 36.034 | 1.035 | 35.060 | 1.00 | 39.05 |
| ATOM | 6706 | CD2 | HIS | 3253 | 34.704 | 0.767 | 35.044 | 1.00 | 39.72 |
| ATOM | 6707 | ND1 | HIS | 3253 | 36.657 | −0.137 | 35.441 | 1.00 | 40.11 |
| ATOM | 6708 | CE1 | HIS | 3253 | 35.744 | −1.070 | 35.650 | 1.00 | 40.59 |
| ATOM | 6709 | NE2 | HIS | 3253 | 34.551 | −0.548 | 35.418 | 1.00 | 40.67 |
| ATOM | 6710 | C | HIS | 3253 | 39.019 | 1.220 | 34.403 | 1.00 | 33.65 |
| ATOM | 6711 | O | HIS | 3253 | 39.206 | 0.094 | 33.923 | 1.00 | 33.50 |
| ATOM | 6712 | N | ARG | 3254 | 39.732 | 1.705 | 35.413 | 1.00 | 32.22 |
| ATOM | 6713 | CA | ARG | 3254 | 40.767 | 0.917 | 36.048 | 1.00 | 30.61 |
| ATOM | 6714 | CB | ARG | 3254 | 41.432 | 1.728 | 37.150 | 1.00 | 31.85 |
| ATOM | 6715 | CG | ARG | 3254 | 40.509 | 2.035 | 38.315 | 1.00 | 32.68 |
| ATOM | 6716 | CD | ARG | 3254 | 41.134 | 3.040 | 39.255 | 1.00 | 34.43 |
| ATOM | 6717 | NE | ARG | 3254 | 40.445 | 3.042 | 40.540 | 1.00 | 38.01 |
| ATOM | 6718 | CZ | ARG | 3254 | 40.942 | 3.585 | 41.646 | 1.00 | 39.20 |
| ATOM | 6719 | NH1 | ARG | 3254 | 42.132 | 4.176 | 41.609 | 1.00 | 40.80 |
| ATOM | 6720 | NH2 | ARG | 3254 | 40.268 | 3.521 | 42.790 | 1.00 | 39.21 |
| ATOM | 6721 | C | ARG | 3254 | 40.102 | −0.312 | 36.647 | 1.00 | 29.38 |
| ATOM | 6722 | O | ARG | 3254 | 38.877 | −0.348 | 36.791 | 1.00 | 28.41 |
| ATOM | 6723 | N | PRO | 3255 | 40.897 | −1.334 | 37.003 | 1.00 | 28.11 |
| ATOM | 6724 | CD | PRO | 3255 | 42.358 | −1.439 | 36.872 | 1.00 | 28.32 |
| ATOM | 6725 | CA | PRO | 3255 | 40.337 | −2.557 | 37.592 | 1.00 | 27.63 |
| ATOM | 6726 | CB | PRO | 3255 | 41.570 | −3.422 | 37.836 | 1.00 | 27.34 |
| ATOM | 6727 | CG | PRO | 3255 | 42.556 | −2.923 | 36.804 | 1.00 | 28.33 |
| ATOM | 6728 | C | PRO | 3255 | 39.598 | −2.268 | 38.895 | 1.00 | 27.91 |
| ATOM | 6729 | O | PRO | 3255 | 39.947 | −1.342 | 39.636 | 1.00 | 27.84 |
| ATOM | 6730 | N | ILE | 3256 | 38.579 | −3.070 | 39.174 | 1.00 | 27.28 |
| ATOM | 6731 | CA | ILE | 3256 | 37.805 | −2.912 | 40.391 | 1.00 | 27.33 |
| ATOM | 6732 | CB | ILE | 3256 | 36.329 | −2.617 | 40.059 | 1.00 | 27.44 |
| ATOM | 6733 | CG2 | ILE | 3256 | 35.467 | −2.705 | 41.306 | 1.00 | 26.97 |
| ATOM | 6734 | CG1 | ILE | 3256 | 36.229 | −1.221 | 39.445 | 1.00 | 27.54 |
| ATOM | 6735 | CD1 | ILE | 3256 | 34.839 | −0.871 | 38.967 | 1.00 | 28.61 |
| ATOM | 6736 | C | ILE | 3256 | 37.933 | −4.182 | 41.219 | 1.00 | 27.59 |
| ATOM | 6737 | O | ILE | 3256 | 37.724 | −5.284 | 40.715 | 1.00 | 28.86 |
| ATOM | 6738 | N | LEU | 3257 | 38.302 | −4.026 | 42.485 | 1.00 | 27.76 |
| ATOM | 6739 | CA | LEU | 3257 | 38.474 | −5.167 | 43.372 | 1.00 | 28.67 |
| ATOM | 6740 | CB | LEU | 3257 | 39.734 | −4.983 | 44.232 | 1.00 | 28.32 |
| ATOM | 6741 | CG | LEU | 3257 | 41.031 | −4.550 | 43.530 | 1.00 | 28.76 |
| ATOM | 6742 | CD1 | LEU | 3257 | 42.201 | −4.723 | 44.464 | 1.00 | 29.95 |
| ATOM | 6743 | CD2 | LEU | 3257 | 41.262 | −5.374 | 42.300 | 1.00 | 29.03 |
| ATOM | 6744 | C | LEU | 3257 | 37.244 | −5.303 | 44.263 | 1.00 | 29.78 |
| ATOM | 6745 | O | LEU | 3257 | 36.726 | −4.305 | 44.765 | 1.00 | 29.93 |
| ATOM | 6746 | N | GLN | 3258 | 36.775 | −6.533 | 44.459 | 1.00 | 30.52 |
| ATOM | 6747 | CA | GLN | 3258 | 35.596 | −6.786 | 45.291 | 1.00 | 31.72 |
| ATOM | 6748 | CB | GLN | 3258 | 35.292 | −8.291 | 45.299 | 1.00 | 31.94 |
| ATOM | 6749 | C | GLN | 3258 | 35.806 | −6.298 | 46.730 | 1.00 | 31.62 |
| ATOM | 6750 | O | GLN | 3258 | 36.744 | −6.712 | 47.398 | 1.00 | 32.20 |
| ATOM | 6751 | N | ALA | 3259 | 34.931 | −5.412 | 47.194 | 1.00 | 31.74 |
| ATOM | 6752 | CA | ALA | 3259 | 35.025 | −4.895 | 48.549 | 1.00 | 31.64 |
| ATOM | 6753 | CB | ALA | 3259 | 33.775 | −4.082 | 48.873 | 1.00 | 30.51 |
| ATOM | 6754 | C | ALA | 3259 | 35.177 | −6.061 | 49.546 | 1.00 | 32.18 |
| ATOM | 6755 | O | ALA | 3259 | 34.571 | −7.123 | 49.371 | 1.00 | 32.29 |
| ATOM | 6756 | N | GLY | 3260 | 36.006 | −5.869 | 50.570 | 1.00 | 32.16 |
| ATOM | 6757 | CA | GLY | 3260 | 36.181 | −6.898 | 51.575 | 1.00 | 32.75 |
| ATOM | 6758 | C | GLY | 3260 | 37.317 | −7.883 | 51.371 | 1.00 | 33.64 |
| ATOM | 6759 | O | GLY | 3260 | 37.694 | −8.574 | 52.319 | 1.00 | 34.98 |
| ATOM | 6760 | N | LEU | 3261 | 37.865 | −7.974 | 50.162 | 1.00 | 33.13 |
| ATOM | 6761 | CA | LEU | 3261 | 38.947 | −8.920 | 49.906 | 1.00 | 32.17 |
| ATOM | 6762 | CB | LEU | 3261 | 38.518 | −9.982 | 48.895 | 1.00 | 33.11 |
| ATOM | 6763 | CG | LEU | 3261 | 37.375 | −10.926 | 49.281 | 1.00 | 33.96 |
| ATOM | 6764 | CD1 | LEU | 3261 | 37.470 | −12.170 | 48.417 | 1.00 | 33.89 |
| ATOM | 6765 | CD2 | LEU | 3261 | 37.468 | −11.313 | 50.746 | 1.00 | 33.03 |
| ATOM | 6766 | C | LEU | 3261 | 40.206 | −8.263 | 49.388 | 1.00 | 31.71 |
| ATOM | 6767 | O | LEU | 3261 | 40.146 | −7.368 | 48.559 | 1.00 | 31.83 |
| ATOM | 6768 | N | PRO | 3262 | 41.375 | −8.695 | 49.887 | 1.00 | 31.76 |
| ATOM | 6769 | CD | PRO | 3262 | 42.674 | −8.201 | 49.404 | 1.00 | 31.72 |
| ATOM | 6770 | CA | PRO | 3262 | 41.567 | −9.745 | 50.895 | 1.00 | 32.18 |
| ATOM | 6771 | CB | PRO | 3262 | 43.072 | −9.964 | 50.873 | 1.00 | 32.29 |
| ATOM | 6772 | CG | PRO | 3262 | 43.598 | −8.614 | 50.527 | 1.00 | 31.74 |
| ATOM | 6773 | C | PRO | 3262 | 41.091 | −9.293 | 52.270 | 1.00 | 32.91 |
| ATOM | 6774 | O | PRO | 3262 | 40.980 | −8.089 | 52.535 | 1.00 | 33.29 |
| ATOM | 6775 | N | ALA | 3263 | 40.835 | −10.253 | 53.153 | 1.00 | 33.36 |
| ATOM | 6776 | CA | ALA | 3263 | 40.366 | −9.937 | 54.500 | 1.00 | 33.49 |
| ATOM | 6777 | CB | ALA | 3263 | 39.123 | −10.756 | 54.822 | 1.00 | 33.17 |
| ATOM | 6778 | C | ALA | 3263 | 41.425 | −10.177 | 55.568 | 1.00 | 33.72 |
| ATOM | 6779 | O | ALA | 3263 | 42.300 | −11.030 | 55.421 | 1.00 | 33.56 |
| ATOM | 6780 | N | ASN | 3264 | 41.335 | −9.421 | 56.653 | 1.00 | 34.59 |
| ATOM | 6781 | CA | ASN | 3264 | 42.272 | −9.570 | 57.755 | 1.00 | 35.59 |
| ATOM | 6782 | CB | ASN | 3264 | 41.970 | −8.549 | 58.851 | 1.00 | 35.75 |
| ATOM | 6783 | CG | ASN | 3264 | 42.041 | −7.114 | 58.354 | 1.00 | 36.48 |
| ATOM | 6784 | OD1 | ASN | 3264 | 42.870 | −6.776 | 57.492 | 1.00 | 36.39 |
| ATOM | 6785 | ND2 | ASN | 3264 | 41.189 | −6.255 | 58.907 | 1.00 | 35.77 |
| ATOM | 6786 | C | ASN | 3264 | 42.153 | −10.980 | 58.326 | 1.00 | 36.61 |
| ATOM | 6787 | O | ASN | 3264 | 41.062 | −11.544 | 58.401 | 1.00 | 37.38 |
| ATOM | 6788 | N | LYS | 3265 | 43.273 | −11.556 | 58.730 | 1.00 | 37.25 |
| ATOM | 6789 | CA | LYS | 3265 | 43.251 | −12.894 | 59.287 | 1.00 | 38.57 |
| ATOM | 6790 | CB | LYS | 3265 | 43.684 | −13.921 | 58.232 | 1.00 | 39.20 |
| ATOM | 6791 | CG | LYS | 3265 | 42.603 | −14.311 | 57.229 | 1.00 | 39.47 |
| ATOM | 6792 | CD | LYS | 3265 | 43.237 | −14.914 | 55.975 | 1.00 | 40.38 |
| ATOM | 6793 | CE | LYS | 3265 | 42.212 | −15.618 | 55.071 | 1.00 | 40.94 |
| ATOM | 6794 | NZ | LYS | 3265 | 41.128 | −14.729 | 54.563 | 1.00 | 40.84 |
| ATOM | 6795 | C | LYS | 3265 | 44.143 | −13.015 | 60.510 | 1.00 | 39.23 |
| ATOM | 6796 | O | LYS | 3265 | 45.242 | −12.462 | 60.562 | 1.00 | 38.94 |
| ATOM | 6797 | N | THR | 3266 | 43.646 | −13.747 | 61.497 | 1.00 | 39.76 |
| ATOM | 6798 | CA | THR | 3266 | 44.372 | −13.993 | 62.725 | 1.00 | 40.06 |
| ATOM | 6799 | CB | THR | 3266 | 43.572 | −13.479 | 63.938 | 1.00 | 39.58 |
| ATOM | 6800 | OG1 | THR | 3266 | 43.477 | −12.047 | 63.868 | 1.00 | 40.05 |
| ATOM | 6801 | CG2 | THR | 3266 | 44.240 | −13.892 | 65.244 | 1.00 | 38.53 |
| ATOM | 6802 | C | THR | 3266 | 44.503 | −15.509 | 62.790 | 1.00 | 40.69 |
| ATOM | 6803 | O | THR | 3266 | 43.504 | −16.211 | 62.879 | 1.00 | 40.94 |
| ATOM | 6804 | N | VAL | 3267 | 45.723 | −16.025 | 62.710 | 1.00 | 40.91 |
| ATOM | 6805 | CA | VAL | 3267 | 45.894 | −17.467 | 62.776 | 1.00 | 41.66 |
| ATOM | 6806 | CB | VAL | 3267 | 46.152 | −18.085 | 61.390 | 1.00 | 41.15 |
| ATOM | 6807 | CG1 | VAL | 3267 | 45.047 | −17.681 | 60.442 | 1.00 | 41.40 |
| ATOM | 6808 | CG2 | VAL | 3267 | 47.524 | −17.662 | 60.867 | 1.00 | 41.44 |
| ATOM | 6809 | C | VAL | 3267 | 47.015 | −17.896 | 63.706 | 1.00 | 42.68 |
| ATOM | 6810 | O | VAL | 3267 | 47.886 | −17.100 | 64.075 | 1.00 | 42.80 |
| ATOM | 6811 | N | ALA | 3268 | 46.975 | −19.170 | 64.086 | 1.00 | 43.67 |
| ATOM | 6812 | CA | ALA | 3268 | 47.971 | −19.740 | 64.977 | 1.00 | 44.57 |
| ATOM | 6813 | CB | ALA | 3268 | 47.428 | −21.008 | 65.622 | 1.00 | 43.49 |
| ATOM | 6814 | C | ALA | 3268 | 49.243 | −20.043 | 64.193 | 1.00 | 45.06 |
| ATOM | 6815 | O | ALA | 3268 | 49.202 | −20.345 | 62.998 | 1.00 | 44.73 |
| ATOM | 6816 | N | LEU | 3269 | 50.375 | −19.950 | 64.872 | 1.00 | 45.95 |
| ATOM | 6817 | CA | LEU | 3269 | 51.653 | −20.216 | 64.233 | 1.00 | 47.35 |
| ATOM | 6818 | CB | LEU | 3269 | 52.769 | −20.180 | 65.281 | 1.00 | 47.80 |
| ATOM | 6819 | CG | LEU | 3269 | 54.112 | −19.616 | 64.818 | 1.00 | 48.41 |
| ATOM | 6820 | CD1 | LEU | 3269 | 55.011 | −19.389 | 66.030 | 1.00 | 48.33 |
| ATOM | 6821 | CD2 | LEU | 3269 | 54.755 | −20.560 | 63.809 | 1.00 | 48.31 |
| ATOM | 6822 | C | LEU | 3269 | 51.596 | −21.587 | 63.564 | 1.00 | 47.95 |
| ATOM | 6823 | O | LEU | 3269 | 50.994 | −22.523 | 64.093 | 1.00 | 48.23 |
| ATOM | 6824 | N | GLY | 3270 | 52.206 | −21.699 | 62.390 | 1.00 | 48.46 |
| ATOM | 6825 | CA | GLY | 3270 | 52.214 | −22.966 | 61.683 | 1.00 | 48.73 |
| ATOM | 6826 | C | GLY | 3270 | 50.971 | −23.257 | 60.861 | 1.00 | 48.82 |
| ATOM | 6827 | O | GLY | 3270 | 50.896 | −24.291 | 60.189 | 1.00 | 48.59 |
| ATOM | 6828 | N | SER | 3271 | 49.992 | −22.357 | 60.904 | 1.00 | 48.74 |
| ATOM | 6829 | CA | SER | 3271 | 48.763 | −22.552 | 60.135 | 1.00 | 48.72 |
| ATOM | 6830 | CB | SER | 3271 | 47.644 | −21.608 | 60.616 | 1.00 | 48.30 |
| ATOM | 6831 | OG | SER | 3271 | 47.312 | −21.786 | 61.983 | 1.00 | 48.90 |
| ATOM | 6832 | C | SER | 3271 | 49.000 | −22.274 | 58.653 | 1.00 | 48.85 |
| ATOM | 6833 | O | SER | 3271 | 50.043 | −21.747 | 58.252 | 1.00 | 48.72 |
| ATOM | 6834 | N | ASN | 3272 | 48.020 | −22.644 | 57.840 | 1.00 | 48.90 |
| ATOM | 6835 | CA | ASN | 3272 | 48.080 | −22.386 | 56.412 | 1.00 | 48.65 |
| ATOM | 6836 | CB | ASN | 3272 | 47.800 | −23.656 | 55.601 | 1.00 | 49.88 |
| ATOM | 6837 | CG | ASN | 3272 | 49.053 | −24.489 | 55.374 | 1.00 | 51.27 |
| ATOM | 6838 | OD1 | ASN | 3272 | 49.648 | −25.012 | 56.318 | 1.00 | 52.43 |
| ATOM | 6839 | ND2 | ASN | 3272 | 49.465 | −24.606 | 54.116 | 1.00 | 52.58 |
| ATOM | 6840 | C | ASN | 3272 | 46.992 | −21.353 | 56.185 | 1.00 | 48.07 |
| ATOM | 6841 | O | ASN | 3272 | 45.898 | −21.457 | 56.748 | 1.00 | 47.84 |
| ATOM | 6842 | N | VAL | 3273 | 47.289 | −20.337 | 55.390 | 1.00 | 47.12 |
| ATOM | 6843 | CA | VAL | 3273 | 46.290 | −19.314 | 55.148 | 1.00 | 46.55 |
| ATOM | 6844 | CB | VAL | 3273 | 46.505 | −18.071 | 56.093 | 1.00 | 47.03 |
| ATOM | 6845 | CG1 | VAL | 3273 | 47.782 | −18.251 | 56.929 | 1.00 | 46.16 |
| ATOM | 6846 | CG2 | VAL | 3273 | 46.565 | −16.770 | 55.280 | 1.00 | 46.33 |
| ATOM | 6847 | C | VAL | 3273 | 46.275 | −18.888 | 53.696 | 1.00 | 46.11 |
| ATOM | 6848 | O | VAL | 3273 | 47.302 | −18.924 | 53.011 | 1.00 | 46.51 |
| ATOM | 6849 | N | GLU | 3274 | 45.095 | −18.514 | 53.217 | 1.00 | 45.35 |
| ATOM | 6850 | CA | GLU | 3274 | 44.988 | −18.056 | 51.850 | 1.00 | 44.83 |
| ATOM | 6851 | CB | GLU | 3274 | 44.421 | −19.156 | 50.926 | 1.00 | 46.52 |
| ATOM | 6852 | CG | GLU | 3274 | 43.091 | −19.780 | 51.325 | 1.00 | 48.74 |
| ATOM | 6853 | CD | GLU | 3274 | 42.653 | −20.897 | 50.363 | 1.00 | 50.69 |
| ATOM | 6854 | OE1 | GLU | 3274 | 43.397 | −21.900 | 50.214 | 1.00 | 50.89 |
| ATOM | 6855 | OE2 | GLU | 3274 | 41.562 | −20.769 | 49.756 | 1.00 | 51.40 |
| ATOM | 6856 | C | GLU | 3274 | 44.189 | −16.768 | 51.745 | 1.00 | 43.56 |
| ATOM | 6857 | O | GLU | 3274 | 43.062 | −16.649 | 52.240 | 1.00 | 42.81 |
| ATOM | 6858 | N | PHE | 3275 | 44.821 | −15.779 | 51.130 | 1.00 | 42.27 |
| ATOM | 6859 | CA | PHE | 3275 | 44.189 | −14.489 | 50.918 | 1.00 | 41.18 |
| ATOM | 6860 | CB | PHE | 3275 | 45.231 | −13.378 | 50.966 | 1.00 | 40.40 |
| ATOM | 6861 | CG | PHE | 3275 | 45.638 | −12.985 | 52.356 | 1.00 | 39.51 |
| ATOM | 6862 | CD1 | PHE | 3275 | 44.736 | −12.342 | 53.203 | 1.00 | 38.97 |
| ATOM | 6863 | CD2 | PHE | 3275 | 46.928 | −13.228 | 52.813 | 1.00 | 38.46 |
| ATOM | 6864 | CE1 | PHE | 3275 | 45.117 | −11.944 | 54.481 | 1.00 | 37.64 |
| ATOM | 6865 | CE2 | PHE | 3275 | 47.313 | −12.832 | 54.093 | 1.00 | 37.76 |
| ATOM | 6866 | CZ | PHE | 3275 | 46.407 | −12.189 | 54.924 | 1.00 | 37.33 |
| ATOM | 6867 | C | PHE | 3275 | 43.534 | −14.530 | 49.546 | 1.00 | 41.21 |
| ATOM | 6868 | O | PHE | 3275 | 44.070 | −15.137 | 48.612 | 1.00 | 40.63 |
| ATOM | 6869 | N | MSE | 3276 | 42.373 | −13.897 | 49.431 | 1.00 | 41.06 |
| ATOM | 6870 | CA | MSE | 3276 | 41.647 | −13.874 | 48.172 | 1.00 | 41.77 |
| ATOM | 6871 | CB | MSE | 3276 | 40.223 | −14.412 | 48.351 | 1.00 | 43.92 |
| ATOM | 6872 | CG | MSE | 3276 | 40.093 | −15.885 | 48.719 | 1.00 | 46.98 |
| ATOM | 6873 | SE | MSE | 3276 | 38.332 | −16.335 | 48.910 | 1.00 | 50.73 |
| ATOM | 6874 | CE | MSE | 3276 | 37.829 | −16.502 | 47.164 | 1.00 | 49.06 |
| ATOM | 6875 | C | MSE | 3276 | 41.541 | −12.463 | 47.613 | 1.00 | 41.30 |
| ATOM | 6876 | O | MSE | 3276 | 41.661 | −11.474 | 48.348 | 1.00 | 40.74 |
| ATOM | 6877 | N | CYS | 3277 | 41.288 | −12.388 | 46.309 | 1.00 | 40.07 |
| ATOM | 6878 | CA | CYS | 3277 | 41.131 | −11.115 | 45.630 | 1.00 | 39.77 |
| ATOM | 6879 | CB | CYS | 3277 | 42.500 | −10.532 | 45.295 | 1.00 | 40.32 |
| ATOM | 6880 | SG | CYS | 3277 | 42.391 | −8.840 | 44.753 | 1.00 | 42.40 |
| ATOM | 6881 | C | CYS | 3277 | 40.315 | −11.275 | 44.344 | 1.00 | 39.04 |
| ATOM | 6882 | O | CYS | 3277 | 40.741 | −11.964 | 43.415 | 1.00 | 38.43 |
| ATOM | 6883 | N | LYS | 3278 | 39.146 | −10.639 | 44.291 | 1.00 | 38.19 |
| ATOM | 6884 | CA | LYS | 3278 | 38.300 | −10.716 | 43.107 | 1.00 | 37.48 |
| ATOM | 6885 | CB | LYS | 3278 | 36.835 | −10.945 | 43.489 | 1.00 | 39.75 |
| ATOM | 6886 | CG | LYS | 3278 | 36.579 | −12.219 | 44.307 | 1.00 | 42.10 |
| ATOM | 6887 | CD | LYS | 3278 | 37.021 | −13.478 | 43.580 | 1.00 | 43.93 |
| ATOM | 6888 | CE | LYS | 3278 | 36.676 | −14.725 | 44.385 | 1.00 | 44.92 |
| ATOM | 6889 | NZ | LYS | 3278 | 37.250 | −15.937 | 43.734 | 1.00 | 46.50 |
| ATOM | 6890 | C | LYS | 3278 | 38.437 | −9.436 | 42.297 | 1.00 | 36.08 |
| ATOM | 6891 | O | LYS | 3278 | 38.115 | −8.340 | 42.761 | 1.00 | 36.47 |
| ATOM | 6892 | N | VAL | 3279 | 38.914 | −9.587 | 41.071 | 1.00 | 34.21 |
| ATOM | 6893 | CA | VAL | 3279 | 39.132 | −8.447 | 40.202 | 1.00 | 32.05 |
| ATOM | 6894 | CB | VAL | 3279 | 40.579 | −8.434 | 39.672 | 1.00 | 31.88 |
| ATOM | 6895 | CG1 | VAL | 3279 | 40.776 | −7.271 | 38.716 | 1.00 | 32.10 |
| ATOM | 6896 | CG2 | VAL | 3279 | 41.550 | −8.339 | 40.826 | 1.00 | 31.09 |
| ATOM | 6897 | C | VAL | 3279 | 38.206 | −8.392 | 39.004 | 1.00 | 31.21 |
| ATOM | 6898 | O | VAL | 3279 | 37.874 | −9.420 | 38.412 | 1.00 | 31.40 |
| ATOM | 6899 | N | TYR | 3280 | 37.784 | −7.179 | 38.660 | 1.00 | 29.95 |
| ATOM | 6900 | CA | TYR | 3280 | 36.943 | −6.955 | 37.493 | 1.00 | 28.28 |
| ATOM | 6901 | CB | TYR | 3280 | 35.593 | −6.330 | 37.850 | 1.00 | 27.93 |
| ATOM | 6902 | CG | TYR | 3280 | 34.823 | −5.906 | 36.607 | 1.00 | 27.24 |
| ATOM | 6903 | CD1 | TYR | 3280 | 34.056 | −6.830 | 35.891 | 1.00 | 28.11 |
| ATOM | 6904 | CE1 | TYR | 3280 | 33.466 | −6.495 | 34.674 | 1.00 | 26.42 |
| ATOM | 6905 | CD2 | TYR | 3280 | 34.968 | −4.619 | 36.070 | 1.00 | 27.29 |
| ATOM | 6906 | CE2 | TYR | 3280 | 34.388 | −4.274 | 34.849 | 1.00 | 26.33 |
| ATOM | 6907 | CZ | TYR | 3280 | 33.645 | −5.221 | 34.158 | 1.00 | 26.90 |
| ATOM | 6908 | OH | TYR | 3280 | 33.123 | −4.921 | 32.922 | 1.00 | 27.24 |
| ATOM | 6909 | C | TYR | 3280 | 37.681 | −5.961 | 36.616 | 1.00 | 27.94 |
| ATOM | 6910 | O | TYR | 3280 | 38.187 | −4.949 | 37.105 | 1.00 | 27.35 |
| ATOM | 6911 | N | SER | 3281 | 37.724 | −6.236 | 35.321 | 1.00 | 27.38 |
| ATOM | 6912 | CA | SER | 3281 | 38.383 | −5.344 | 34.397 | 1.00 | 27.23 |
| ATOM | 6913 | CB | SER | 3281 | 39.886 | −5.363 | 34.657 | 1.00 | 26.81 |
| ATOM | 6914 | OG | SER | 3281 | 40.575 | −4.517 | 33.772 | 1.00 | 25.62 |
| ATOM | 6915 | C | SER | 3281 | 38.067 | −5.782 | 32.975 | 1.00 | 27.95 |
| ATOM | 6916 | O | SER | 3281 | 38.168 | −6.960 | 32.647 | 1.00 | 27.99 |
| ATOM | 6917 | N | ASP | 3282 | 37.651 | −4.824 | 32.150 | 1.00 | 28.95 |
| ATOM | 6918 | CA | ASP | 3282 | 37.319 | −5.063 | 30.751 | 1.00 | 29.12 |
| ATOM | 6919 | CB | ASP | 3282 | 36.653 | −3.801 | 30.194 | 1.00 | 29.30 |
| ATOM | 6920 | CG | ASP | 3282 | 36.013 | −4.010 | 28.841 | 1.00 | 31.43 |
| ATOM | 6921 | OD1 | ASP | 3282 | 35.183 | −3.147 | 28.478 | 1.00 | 33.06 |
| ATOM | 6922 | OD2 | ASP | 3282 | 36.328 | −5.000 | 28.136 | 1.00 | 30.58 |
| ATOM | 6923 | C | ASP | 3282 | 38.661 | −5.359 | 30.072 | 1.00 | 29.46 |
| ATOM | 6924 | O | ASP | 3282 | 38.901 | −6.468 | 29.600 | 1.00 | 29.59 |
| ATOM | 6925 | N | PRO | 3283 | 39.563 | −4.370 | 30.024 | 1.00 | 29.66 |
| ATOM | 6926 | CD | PRO | 3283 | 39.554 | −3.000 | 30.555 | 1.00 | 29.71 |
| ATOM | 6927 | CA | PRO | 3283 | 40.846 | −4.678 | 29.388 | 1.00 | 30.05 |
| ATOM | 6928 | CB | PRO | 3283 | 41.600 | −3.348 | 29.444 | 1.00 | 30.14 |
| ATOM | 6929 | CG | PRO | 3283 | 40.513 | −2.320 | 29.644 | 1.00 | 30.25 |
| ATOM | 6930 | C | PRO | 3283 | 41.507 | −5.707 | 30.303 | 1.00 | 30.59 |
| ATOM | 6931 | O | PRO | 3283 | 41.269 | −5.701 | 31.514 | 1.00 | 31.46 |
| ATOM | 6932 | N | GLN | 3284 | 42.334 | −6.576 | 29.737 | 1.00 | 30.53 |
| ATOM | 6933 | CA | GLN | 3284 | 43.031 | −7.598 | 30.512 | 1.00 | 30.27 |
| ATOM | 6934 | CB | GLN | 3284 | 43.968 | −8.363 | 29.566 | 1.00 | 30.53 |
| ATOM | 6935 | CG | GLN | 3284 | 43.982 | −9.869 | 29.732 | 1.00 | 29.97 |
| ATOM | 6936 | CD | GLN | 3284 | 42.603 | −10.474 | 29.774 | 1.00 | 29.67 |
| ATOM | 6937 | OE1 | GLN | 3284 | 41.816 | −10.327 | 28.848 | 1.00 | 29.38 |
| ATOM | 6938 | NE2 | GLN | 3284 | 42.302 | −11.164 | 30.862 | 1.00 | 30.61 |
| ATOM | 6939 | C | GLN | 3284 | 43.822 | −6.937 | 31.656 | 1.00 | 30.59 |
| ATOM | 6940 | O | GLN | 3284 | 44.633 | −6.034 | 31.430 | 1.00 | 31.04 |
| ATOM | 6941 | N | PRO | 3285 | 43.577 | −7.357 | 32.906 | 1.00 | 30.70 |
| ATOM | 6942 | CD | PRO | 3285 | 42.404 | −8.123 | 33.371 | 1.00 | 31.47 |
| ATOM | 6943 | CA | PRO | 3285 | 44.284 | −6.774 | 34.050 | 1.00 | 31.11 |
| ATOM | 6944 | CB | PRO | 3285 | 43.234 | −6.820 | 35.144 | 1.00 | 31.52 |
| ATOM | 6945 | CG | PRO | 3285 | 42.585 | −8.135 | 34.880 | 1.00 | 30.63 |
| ATOM | 6946 | C | PRO | 3285 | 45.528 | −7.549 | 34.458 | 1.00 | 31.70 |
| ATOM | 6947 | O | PRO | 3285 | 45.613 | −8.766 | 34.265 | 1.00 | 31.22 |
| ATOM | 6948 | N | HIS | 3286 | 46.493 | −6.841 | 35.031 | 1.00 | 32.16 |
| ATOM | 6949 | CA | HIS | 3286 | 47.701 | −7.491 | 35.493 | 1.00 | 32.56 |
| ATOM | 6950 | CB | HIS | 3286 | 48.941 | −6.783 | 34.957 | 1.00 | 34.45 |
| ATOM | 6951 | CG | HIS | 3286 | 50.208 | −7.435 | 35.398 | 1.00 | 36.00 |
| ATOM | 6952 | CD2 | HIS | 3286 | 50.845 | −8.536 | 34.938 | 1.00 | 36.27 |
| ATOM | 6953 | ND1 | HIS | 3286 | 50.888 | −7.040 | 36.529 | 1.00 | 36.06 |
| ATOM | 6954 | CE1 | HIS | 3286 | 51.890 | −7.872 | 36.749 | 1.00 | 36.78 |
| ATOM | 6955 | NE2 | HIS | 3286 | 51.886 | −8.788 | 35.798 | 1.00 | 37.27 |
| ATOM | 6956 | C | HIS | 3286 | 47.698 | −7.463 | 37.012 | 1.00 | 32.12 |
| ATOM | 6957 | O | HIS | 3286 | 47.817 | −6.400 | 37.614 | 1.00 | 32.46 |
| ATOM | 6958 | N | ILE | 3287 | 47.548 | −8.634 | 37.628 | 1.00 | 32.16 |
| ATOM | 6959 | CA | ILE | 3287 | 47.510 | −8.745 | 39.085 | 1.00 | 31.95 |
| ATOM | 6960 | CB | ILE | 3287 | 46.573 | −9.852 | 39.531 | 1.00 | 31.64 |
| ATOM | 6961 | CG2 | ILE | 3287 | 46.545 | −9.931 | 41.058 | 1.00 | 30.67 |
| ATOM | 6962 | CG1 | ILE | 3287 | 45.178 | −9.590 | 38.970 | 1.00 | 31.34 |
| ATOM | 6963 | CD1 | ILE | 3287 | 44.206 | −10.703 | 39.260 | 1.00 | 32.74 |
| ATOM | 6964 | C | ILE | 3287 | 48.874 | −9.042 | 39.676 | 1.00 | 32.77 |
| ATOM | 6965 | O | ILE | 3287 | 49.720 | −9.656 | 39.032 | 1.00 | 33.35 |
| ATOM | 6966 | N | GLN | 3288 | 49.074 | −8.619 | 40.917 | 1.00 | 33.63 |
| ATOM | 6967 | CA | GLN | 3288 | 50.341 | −8.825 | 41.599 | 1.00 | 34.86 |
| ATOM | 6968 | CB | GLN | 3288 | 51.298 | −7.693 | 41.204 | 1.00 | 36.46 |
| ATOM | 6969 | CG | GLN | 3288 | 52.697 | −7.788 | 41.768 | 1.00 | 39.66 |
| ATOM | 6970 | CD | GLN | 3288 | 53.743 | −7.160 | 40.843 | 1.00 | 41.71 |
| ATOM | 6971 | OE1 | GLN | 3288 | 54.121 | −7.747 | 39.819 | 1.00 | 42.14 |
| ATOM | 6972 | NE2 | GLN | 3288 | 54.213 | −5.963 | 41.201 | 1.00 | 43.11 |
| ATOM | 6973 | C | GLN | 3288 | 50.089 | −8.837 | 43.104 | 1.00 | 34.46 |
| ATOM | 6974 | O | GLN | 3288 | 49.286 | −8.045 | 43.597 | 1.00 | 34.66 |
| ATOM | 6975 | N | TRP | 3289 | 50.740 | −9.752 | 43.825 | 1.00 | 34.46 |
| ATOM | 6976 | CA | TRP | 3289 | 50.586 | −9.831 | 45.289 | 1.00 | 33.83 |
| ATOM | 6977 | CB | TRP | 3289 | 50.315 | −11.257 | 45.754 | 1.00 | 33.44 |
| ATOM | 6978 | CG | TRP | 3289 | 48.938 | −11.742 | 45.546 | 1.00 | 31.95 |
| ATOM | 6979 | CD2 | TRP | 3289 | 47.832 | −11.581 | 46.444 | 1.00 | 32.13 |
| ATOM | 6980 | CE2 | TRP | 3289 | 46.743 | −12.275 | 45.889 | 1.00 | 31.46 |
| ATOM | 6981 | CE3 | TRP | 3289 | 47.659 | −10.918 | 47.668 | 1.00 | 31.70 |
| ATOM | 6982 | CD1 | TRP | 3289 | 48.486 | −12.490 | 44.508 | 1.00 | 31.70 |
| ATOM | 6983 | NE1 | TRP | 3289 | 47.167 | −12.823 | 44.707 | 1.00 | 32.11 |
| ATOM | 6984 | CZ2 | TRP | 3289 | 45.493 | −12.328 | 46.516 | 1.00 | 31.24 |
| ATOM | 6985 | CZ3 | TRP | 3289 | 46.417 | −10.973 | 48.292 | 1.00 | 30.83 |
| ATOM | 6986 | CH2 | TRP | 3289 | 45.353 | −11.673 | 47.714 | 1.00 | 30.77 |
| ATOM | 6987 | C | TRP | 3289 | 51.861 | −9.368 | 45.960 | 1.00 | 33.85 |
| ATOM | 6988 | O | TRP | 3289 | 52.941 | −9.866 | 45.638 | 1.00 | 33.32 |
| ATOM | 6989 | N | LEU | 3290 | 51.736 | −8.424 | 46.892 | 1.00 | 34.60 |
| ATOM | 6990 | CA | LEU | 3290 | 52.897 | −7.895 | 47.601 | 1.00 | 35.49 |
| ATOM | 6991 | CB | LEU | 3290 | 53.097 | −6.398 | 47.336 | 1.00 | 35.97 |
| ATOM | 6992 | CG | LEU | 3290 | 53.162 | −5.781 | 45.936 | 1.00 | 36.73 |
| ATOM | 6993 | CD1 | LEU | 3290 | 53.933 | −6.700 | 45.001 | 1.00 | 36.48 |
| ATOM | 6994 | CD2 | LEU | 3290 | 51.758 | −5.544 | 45.416 | 1.00 | 36.71 |
| ATOM | 6995 | C | LEU | 3290 | 52.777 | −8.065 | 49.096 | 1.00 | 36.29 |
| ATOM | 6996 | O | LEU | 3290 | 51.673 | −8.127 | 49.641 | 1.00 | 35.88 |
| ATOM | 6997 | N | LYS | 3291 | 53.933 | −8.128 | 49.748 | 1.00 | 37.68 |
| ATOM | 6998 | CA | LYS | 3291 | 54.013 | −8.245 | 51.191 | 1.00 | 39.41 |
| ATOM | 6999 | CB | LYS | 3291 | 54.741 | −9.534 | 51.575 | 1.00 | 40.34 |
| ATOM | 7000 | CG | LYS | 3291 | 54.620 | −9.909 | 53.053 | 1.00 | 42.31 |
| ATOM | 7001 | CD | LYS | 3291 | 55.531 | −9.077 | 53.949 | 1.00 | 43.09 |
| ATOM | 7002 | CE | LYS | 3291 | 55.085 | −9.175 | 55.420 | 1.00 | 44.47 |
| ATOM | 7003 | NZ | LYS | 3291 | 54.861 | −10.580 | 55.894 | 1.00 | 43.64 |
| ATOM | 7004 | C | LYS | 3291 | 54.810 | −7.031 | 51.652 | 1.00 | 40.40 |
| ATOM | 7005 | O | LYS | 3291 | 55.912 | −6.802 | 51.177 | 1.00 | 40.33 |
| ATOM | 7006 | N | HIS | 3292 | 54.249 | −6.237 | 52.554 | 1.00 | 42.24 |
| ATOM | 7007 | CA | HIS | 3292 | 54.956 | −5.065 | 53.046 | 1.00 | 44.33 |
| ATOM | 7008 | CB | HIS | 3292 | 53.985 | −4.105 | 53.730 | 1.00 | 45.41 |
| ATOM | 7009 | CG | HIS | 3292 | 53.057 | −3.408 | 52.786 | 1.00 | 45.96 |
| ATOM | 7010 | CD2 | HIS | 3292 | 52.210 | −3.892 | 51.847 | 1.00 | 46.66 |
| ATOM | 7011 | ND1 | HIS | 3292 | 52.956 | −2.034 | 52.723 | 1.00 | 46.53 |
| ATOM | 7012 | CE1 | HIS | 3292 | 52.090 | −1.701 | 51.781 | 1.00 | 46.80 |
| ATOM | 7013 | NE2 | HIS | 3292 | 51.623 | −2.810 | 51.234 | 1.00 | 46.85 |
| ATOM | 7014 | C | HIS | 3292 | 56.037 | −5.489 | 54.023 | 1.00 | 46.19 |
| ATOM | 7015 | O | HIS | 3292 | 55.760 | −6.193 | 54.988 | 1.00 | 45.93 |
| ATOM | 7016 | N | ILE | 3293 | 57.267 | −5.053 | 53.764 | 1.00 | 49.59 |
| ATOM | 7017 | CA | ILE | 3293 | 58.405 | −5.389 | 54.616 | 1.00 | 53.02 |
| ATOM | 7018 | CB | ILE | 3293 | 59.402 | −6.282 | 53.864 | 1.00 | 52.91 |
| ATOM | 7019 | CG2 | ILE | 3293 | 58.699 | −7.539 | 53.373 | 1.00 | 52.62 |
| ATOM | 7020 | CG1 | ILE | 3293 | 60.022 | −5.496 | 52.702 | 1.00 | 52.76 |
| ATOM | 7021 | CD1 | ILE | 3293 | 61.054 | −6.271 | 51.908 | 1.00 | 52.36 |
| ATOM | 7022 | C | ILE | 3293 | 59.152 | −4.143 | 55.099 | 1.00 | 55.69 |
| ATOM | 7023 | O | ILE | 3293 | 59.164 | −3.115 | 54.421 | 1.00 | 56.08 |
| ATOM | 7024 | N | GLU | 3294 | 59.792 | −4.246 | 56.262 | 1.00 | 58.88 |
| ATOM | 7025 | CA | GLU | 3294 | 60.538 | −3.122 | 56.834 | 1.00 | 62.18 |
| ATOM | 7026 | CB | GLU | 3294 | 60.032 | −2.832 | 58.254 | 1.00 | 62.23 |
| ATOM | 7027 | C | GLU | 3294 | 62.046 | −3.381 | 56.879 | 1.00 | 64.20 |
| ATOM | 7028 | O | GLU | 3294 | 62.509 | −4.188 | 57.684 | 1.00 | 64.69 |
| ATOM | 7029 | N | VAL | 3295 | 62.809 | −2.694 | 56.026 | 1.00 | 66.44 |
| ATOM | 7030 | CA | VAL | 3295 | 64.266 | −2.863 | 55.990 | 1.00 | 68.86 |
| ATOM | 7031 | CB | VAL | 3295 | 64.938 | −1.780 | 55.121 | 1.00 | 68.91 |
| ATOM | 7032 | CG1 | VAL | 3295 | 66.452 | −1.943 | 55.162 | 1.00 | 68.37 |
| ATOM | 7033 | CG2 | VAL | 3295 | 64.434 | −1.878 | 53.695 | 1.00 | 69.00 |
| ATOM | 7034 | C | VAL | 3295 | 64.875 | −2.792 | 57.395 | 1.00 | 70.80 |
| ATOM | 7035 | O | VAL | 3295 | 65.873 | −3.457 | 57.694 | 1.00 | 71.06 |
| ATOM | 7036 | N | ASN | 3296 | 64.268 | −1.970 | 58.245 | 1.00 | 72.76 |
| ATOM | 7037 | CA | ASN | 3296 | 64.696 | −1.783 | 59.631 | 1.00 | 74.37 |
| ATOM | 7038 | CB | ASN | 3296 | 65.832 | −0.758 | 59.696 | 1.00 | 74.54 |
| ATOM | 7039 | CG | ASN | 3296 | 66.964 | −1.082 | 58.738 | 1.00 | 75.07 |
| ATOM | 7040 | OD1 | ASN | 3296 | 67.685 | −2.067 | 58.914 | 1.00 | 75.19 |
| ATOM | 7041 | ND2 | ASN | 3296 | 67.119 | −0.257 | 57.707 | 1.00 | 75.31 |
| ATOM | 7042 | C | ASN | 3296 | 63.455 | −1.232 | 60.330 | 1.00 | 75.29 |
| ATOM | 7043 | O | ASN | 3296 | 62.334 | −1.662 | 60.037 | 1.00 | 75.49 |
| ATOM | 7044 | N | GLY | 3297 | 63.636 | −0.295 | 61.252 | 1.00 | 75.99 |
| ATOM | 7045 | CA | GLY | 3297 | 62.469 | 0.290 | 61.883 | 1.00 | 77.04 |
| ATOM | 7046 | C | GLY | 3297 | 61.794 | 1.036 | 60.743 | 1.00 | 77.74 |
| ATOM | 7047 | O | GLY | 3297 | 60.601 | 1.357 | 60.778 | 1.00 | 77.53 |
| ATOM | 7048 | N | SER | 3298 | 62.594 | 1.295 | 59.709 | 1.00 | 78.41 |
| ATOM | 7049 | CA | SER | 3298 | 62.145 | 1.997 | 58.516 | 1.00 | 79.25 |
| ATOM | 7050 | CB | SER | 3298 | 63.346 | 2.569 | 57.750 | 1.00 | 79.01 |
| ATOM | 7051 | OG | SER | 3298 | 64.201 | 1.538 | 57.284 | 1.00 | 79.25 |
| ATOM | 7052 | C | SER | 3298 | 61.345 | 1.086 | 57.593 | 1.00 | 79.83 |
| ATOM | 7053 | O | SER | 3298 | 61.901 | 0.244 | 56.878 | 1.00 | 80.13 |
| ATOM | 7054 | N | LYS | 3299 | 60.029 | 1.255 | 57.625 | 1.00 | 80.07 |
| ATOM | 7055 | CA | LYS | 3299 | 59.148 | 0.479 | 56.772 | 1.00 | 80.29 |
| ATOM | 7056 | CB | LYS | 3299 | 57.745 | 0.411 | 57.386 | 1.00 | 80.52 |
| ATOM | 7057 | CG | LYS | 3299 | 57.707 | −0.197 | 58.786 | 1.00 | 80.89 |
| ATOM | 7058 | CD | LYS | 3299 | 56.288 | −0.254 | 59.344 | 1.00 | 81.10 |
| ATOM | 7059 | CE | LYS | 3299 | 56.259 | −0.882 | 60.735 | 1.00 | 80.99 |
| ATOM | 7060 | NZ | LYS | 3299 | 54.883 | −0.927 | 61.305 | 1.00 | 80.65 |
| ATOM | 7061 | C | LYS | 3299 | 59.112 | 1.212 | 55.433 | 1.00 | 80.16 |
| ATOM | 7062 | O | LYS | 3299 | 59.013 | 0.594 | 54.375 | 1.00 | 80.40 |
| ATOM | 7063 | N | ILE | 3300 | 59.213 | 2.538 | 55.501 | 1.00 | 79.98 |
| ATOM | 7064 | CA | ILE | 3300 | 59.200 | 3.395 | 54.317 | 1.00 | 79.69 |
| ATOM | 7065 | CB | ILE | 3300 | 58.723 | 4.825 | 54.671 | 1.00 | 79.63 |
| ATOM | 7066 | CG2 | ILE | 3300 | 58.594 | 5.667 | 53.401 | 1.00 | 79.72 |
| ATOM | 7067 | CG1 | ILE | 3300 | 57.390 | 4.765 | 55.426 | 1.00 | 79.41 |
| ATOM | 7068 | CD1 | ILE | 3300 | 56.253 | 4.116 | 54.657 | 1.00 | 79.32 |
| ATOM | 7069 | C | ILE | 3300 | 60.608 | 3.487 | 53.733 | 1.00 | 79.48 |
| ATOM | 7070 | O | ILE | 3300 | 61.594 | 3.289 | 54.448 | 1.00 | 79.39 |
| ATOM | 7071 | N | GLY | 3301 | 60.693 | 3.794 | 52.438 | 1.00 | 79.23 |
| ATOM | 7072 | CA | GLY | 3301 | 61.984 | 3.901 | 51.776 | 1.00 | 78.75 |
| ATOM | 7073 | C | GLY | 3301 | 62.391 | 5.314 | 51.396 | 1.00 | 78.43 |
| ATOM | 7074 | O | GLY | 3301 | 61.613 | 6.254 | 51.576 | 1.00 | 78.22 |
| ATOM | 7075 | N | PRO | 3302 | 63.617 | 5.495 | 50.869 | 1.00 | 78.33 |
| ATOM | 7076 | CD | PRO | 3302 | 64.630 | 4.432 | 50.715 | 1.00 | 78.24 |
| ATOM | 7077 | CA | PRO | 3302 | 64.163 | 6.794 | 50.450 | 1.00 | 78.05 |
| ATOM | 7078 | CB | PRO | 3302 | 65.496 | 6.409 | 49.816 | 1.00 | 78.14 |
| ATOM | 7079 | CG | PRO | 3302 | 65.918 | 5.221 | 50.645 | 1.00 | 78.20 |
| ATOM | 7080 | C | PRO | 3302 | 63.249 | 7.558 | 49.486 | 1.00 | 77.82 |
| ATOM | 7081 | O | PRO | 3302 | 63.168 | 8.789 | 49.530 | 1.00 | 77.68 |
| ATOM | 7082 | N | ASP | 3303 | 62.566 | 6.818 | 48.618 | 1.00 | 77.59 |
| ATOM | 7083 | CA | ASP | 3303 | 61.641 | 7.399 | 47.648 | 1.00 | 77.04 |
| ATOM | 7084 | CB | ASP | 3303 | 61.424 | 6.428 | 46.488 | 1.00 | 77.64 |
| ATOM | 7085 | CG | ASP | 3303 | 61.114 | 5.012 | 46.960 | 1.00 | 78.74 |
| ATOM | 7086 | OD1 | ASP | 3303 | 60.674 | 4.188 | 46.129 | 1.00 | 79.08 |
| ATOM | 7087 | OD2 | ASP | 3303 | 61.319 | 4.717 | 48.161 | 1.00 | 78.84 |
| ATOM | 7088 | C | ASP | 3303 | 60.294 | 7.717 | 48.302 | 1.00 | 76.37 |
| ATOM | 7089 | O | ASP | 3303 | 59.315 | 8.013 | 47.615 | 1.00 | 76.40 |
| ATOM | 7090 | N | ASN | 3304 | 60.258 | 7.650 | 49.632 | 1.00 | 75.35 |
| ATOM | 7091 | CA | ASN | 3304 | 59.055 | 7.923 | 50.419 | 1.00 | 74.01 |
| ATOM | 7092 | CB | ASN | 3304 | 58.443 | 9.271 | 50.015 | 1.00 | 74.62 |
| ATOM | 7093 | CG | ASN | 3304 | 57.289 | 9.686 | 50.919 | 1.00 | 75.31 |
| ATOM | 7094 | OD1 | ASN | 3304 | 57.410 | 9.673 | 52.150 | 1.00 | 74.78 |
| ATOM | 7095 | ND2 | ASN | 3304 | 56.165 | 10.067 | 50.309 | 1.00 | 75.48 |
| ATOM | 7096 | C | ASN | 3304 | 58.003 | 6.819 | 50.310 | 1.00 | 72.60 |
| ATOM | 7097 | O | ASN | 3304 | 56.999 | 6.839 | 51.022 | 1.00 | 72.47 |
| ATOM | 7098 | N | LEU | 3305 | 58.231 | 5.858 | 49.419 | 1.00 | 70.90 |
| ATOM | 7099 | CA | LEU | 3305 | 57.298 | 4.749 | 49.253 | 1.00 | 69.05 |
| ATOM | 7100 | CB | LEU | 3305 | 57.250 | 4.277 | 47.795 | 1.00 | 68.83 |
| ATOM | 7101 | CG | LEU | 3305 | 56.744 | 5.246 | 46.721 | 1.00 | 68.36 |
| ATOM | 7102 | CD1 | LEU | 3305 | 55.696 | 6.181 | 47.310 | 1.00 | 67.98 |
| ATOM | 7103 | CD2 | LEU | 3305 | 57.907 | 6.042 | 46.175 | 1.00 | 68.66 |
| ATOM | 7104 | C | LEU | 3305 | 57.708 | 3.586 | 50.152 | 1.00 | 67.70 |
| ATOM | 7105 | O | LEU | 3305 | 58.886 | 3.420 | 50.473 | 1.00 | 67.84 |
| ATOM | 7106 | N | PRO | 3306 | 56.731 | 2.766 | 50.575 | 1.00 | 65.95 |
| ATOM | 7107 | CD | PRO | 3306 | 55.285 | 2.974 | 50.366 | 1.00 | 65.41 |
| ATOM | 7108 | CA | PRO | 3306 | 56.972 | 1.608 | 51.445 | 1.00 | 64.27 |
| ATOM | 7109 | CB | PRO | 3306 | 55.570 | 1.274 | 51.955 | 1.00 | 64.68 |
| ATOM | 7110 | CG | PRO | 3306 | 54.701 | 1.646 | 50.793 | 1.00 | 64.90 |
| ATOM | 7111 | C | PRO | 3306 | 57.635 | 0.415 | 50.747 | 1.00 | 62.31 |
| ATOM | 7112 | O | PRO | 3306 | 57.341 | 0.125 | 49.585 | 1.00 | 62.30 |
| ATOM | 7113 | N | TYR | 3307 | 58.528 | −0.273 | 51.457 | 1.00 | 59.98 |
| ATOM | 7114 | CA | TYR | 3307 | 59.205 | −1.436 | 50.887 | 1.00 | 57.51 |
| ATOM | 7115 | CB | TYR | 3307 | 60.361 | −1.900 | 51.772 | 1.00 | 59.68 |
| ATOM | 7116 | CG | TYR | 3307 | 61.474 | −0.884 | 51.923 | 1.00 | 62.15 |
| ATOM | 7117 | CD1 | TYR | 3307 | 61.439 | 0.071 | 52.942 | 1.00 | 63.18 |
| ATOM | 7118 | CE1 | TYR | 3307 | 62.465 | 1.006 | 53.091 | 1.00 | 63.93 |
| ATOM | 7119 | CD2 | TYR | 3307 | 62.566 | −0.877 | 51.048 | 1.00 | 62.88 |
| ATOM | 7120 | CE2 | TYR | 3307 | 63.598 | 0.059 | 51.186 | 1.00 | 63.66 |
| ATOM | 7121 | CZ | TYR | 3307 | 63.539 | 0.996 | 52.212 | 1.00 | 64.25 |
| ATOM | 7122 | OH | TYR | 3307 | 64.549 | 1.920 | 52.365 | 1.00 | 64.71 |
| ATOM | 7123 | C | TYR | 3307 | 58.219 | −2.575 | 50.719 | 1.00 | 54.35 |
| ATOM | 7124 | O | TYR | 3307 | 57.352 | −2.788 | 51.558 | 1.00 | 53.91 |
| ATOM | 7125 | N | VAL | 3308 | 58.370 | −3.317 | 49.632 | 1.00 | 50.61 |
| ATOM | 7126 | CA | VAL | 3308 | 57.474 | −4.418 | 49.335 | 1.00 | 47.32 |
| ATOM | 7127 | CB | VAL | 3308 | 56.374 | −3.959 | 48.364 | 1.00 | 46.97 |
| ATOM | 7128 | CG1 | VAL | 3308 | 55.379 | −3.062 | 49.088 | 1.00 | 45.81 |
| ATOM | 7129 | CG2 | VAL | 3308 | 56.999 | −3.211 | 47.204 | 1.00 | 45.78 |
| ATOM | 7130 | C | VAL | 3308 | 58.210 | −5.585 | 48.711 | 1.00 | 46.28 |
| ATOM | 7131 | O | VAL | 3308 | 59.282 | −5.415 | 48.142 | 1.00 | 46.54 |
| ATOM | 7132 | N | GLN | 3309 | 57.627 | −6.771 | 48.816 | 1.00 | 45.01 |
| ATOM | 7133 | CA | GLN | 3309 | 58.225 | −7.962 | 48.249 | 1.00 | 44.02 |
| ATOM | 7134 | CB | GLN | 3309 | 58.601 | −8.932 | 49.366 | 1.00 | 45.74 |
| ATOM | 7135 | CG | GLN | 3309 | 59.594 | −10.024 | 48.969 | 1.00 | 47.62 |
| ATOM | 7136 | CD | GLN | 3309 | 59.662 | −11.135 | 50.011 | 1.00 | 49.24 |
| ATOM | 7137 | OE1 | GLN | 3309 | 59.963 | −10.888 | 51.184 | 1.00 | 50.26 |
| ATOM | 7138 | NE2 | GLN | 3309 | 59.376 | −12.365 | 49.587 | 1.00 | 49.10 |
| ATOM | 7139 | C | GLN | 3309 | 57.179 | −8.580 | 47.330 | 1.00 | 42.99 |
| ATOM | 7140 | O | GLN | 3309 | 56.058 | −8.875 | 47.769 | 1.00 | 43.03 |
| ATOM | 7141 | N | ILE | 3310 | 57.528 | −8.749 | 46.055 | 1.00 | 41.35 |
| ATOM | 7142 | CA | ILE | 3310 | 56.599 | −9.338 | 45.097 | 1.00 | 40.25 |
| ATOM | 7143 | CB | ILE | 3310 | 57.080 | −9.166 | 43.650 | 1.00 | 41.04 |
| ATOM | 7144 | CG2 | ILE | 3310 | 55.925 | −9.424 | 42.696 | 1.00 | 41.19 |
| ATOM | 7145 | CG1 | ILE | 3310 | 57.637 | −7.753 | 43.430 | 1.00 | 41.77 |
| ATOM | 7146 | CD1 | ILE | 3310 | 56.600 | −6.678 | 43.306 | 1.00 | 42.24 |
| ATOM | 7147 | C | ILE | 3310 | 56.498 | −10.828 | 45.401 | 1.00 | 39.96 |
| ATOM | 7148 | O | ILE | 3310 | 57.478 | −11.561 | 45.312 | 1.00 | 40.25 |
| ATOM | 7149 | N | LEU | 3311 | 55.304 | −11.276 | 45.759 | 1.00 | 39.51 |
| ATOM | 7150 | CA | LEU | 3311 | 55.095 | −12.667 | 46.108 | 1.00 | 38.97 |
| ATOM | 7151 | CB | LEU | 3311 | 54.140 | −12.759 | 47.288 | 1.00 | 39.43 |
| ATOM | 7152 | CG | LEU | 3311 | 54.497 | −11.941 | 48.523 | 1.00 | 39.54 |
| ATOM | 7153 | CD1 | LEU | 3311 | 53.486 | −12.251 | 49.630 | 1.00 | 39.14 |
| ATOM | 7154 | CD2 | LEU | 3311 | 55.912 | −12.275 | 48.964 | 1.00 | 38.54 |
| ATOM | 7155 | C | LEU | 3311 | 54.519 | −13.484 | 44.974 | 1.00 | 39.11 |
| ATOM | 7156 | O | LEU | 3311 | 54.683 | −14.707 | 44.942 | 1.00 | 39.21 |
| ATOM | 7157 | N | LYS | 3312 | 53.848 | −12.817 | 44.043 | 1.00 | 38.74 |
| ATOM | 7158 | CA | LYS | 3312 | 53.205 | −13.525 | 42.943 | 1.00 | 38.70 |
| ATOM | 7159 | CB | LYS | 3312 | 51.888 | −14.115 | 43.455 | 1.00 | 39.20 |
| ATOM | 7160 | CG | LYS | 3312 | 51.449 | −15.478 | 42.938 | 1.00 | 39.45 |
| ATOM | 7161 | CD | LYS | 3312 | 50.104 | −15.809 | 43.608 | 1.00 | 40.94 |
| ATOM | 7162 | CE | LYS | 3312 | 49.697 | −17.280 | 43.553 | 1.00 | 42.05 |
| ATOM | 7163 | NZ | LYS | 3312 | 49.386 | −17.752 | 42.182 | 1.00 | 44.43 |
| ATOM | 7164 | C | LYS | 3312 | 52.925 | −12.502 | 41.852 | 1.00 | 38.91 |
| ATOM | 7165 | O | LYS | 3312 | 52.396 | −11.420 | 42.131 | 1.00 | 39.47 |
| ATOM | 7166 | N | THR | 3313 | 53.264 | −12.840 | 40.613 | 1.00 | 38.80 |
| ATOM | 7167 | CA | THR | 3313 | 53.059 | −11.921 | 39.496 | 1.00 | 38.76 |
| ATOM | 7168 | CB | THR | 3313 | 54.395 | −11.323 | 39.048 | 1.00 | 39.34 |
| ATOM | 7169 | OG1 | THR | 3313 | 55.086 | −10.820 | 40.196 | 1.00 | 41.50 |
| ATOM | 7170 | CG2 | THR | 3313 | 54.181 | −10.191 | 38.065 | 1.00 | 39.46 |
| ATOM | 7171 | C | THR | 3313 | 52.434 | −12.617 | 38.305 | 1.00 | 38.39 |
| ATOM | 7172 | O | THR | 3313 | 52.948 | −13.634 | 37.843 | 1.00 | 39.52 |
| ATOM | 7173 | N | ALA | 3314 | 51.329 | −12.073 | 37.802 | 1.00 | 37.51 |
| ATOM | 7174 | CA | ALA | 3314 | 50.662 | −12.676 | 36.656 | 1.00 | 37.04 |
| ATOM | 7175 | CB | ALA | 3314 | 49.335 | −11.988 | 36.404 | 1.00 | 36.65 |
| ATOM | 7176 | C | ALA | 3314 | 51.558 | −12.590 | 35.417 | 1.00 | 36.40 |
| ATOM | 7177 | O | ALA | 3314 | 52.370 | −11.674 | 35.280 | 1.00 | 36.33 |
| ATOM | 7178 | N | GLY | 3315 | 51.410 | −13.562 | 34.528 | 1.00 | 35.90 |
| ATOM | 7179 | CA | GLY | 3315 | 52.199 | −13.596 | 33.311 | 1.00 | 36.63 |
| ATOM | 7180 | C | GLY | 3315 | 51.986 | −14.947 | 32.667 | 1.00 | 37.06 |
| ATOM | 7181 | O | GLY | 3315 | 51.132 | −15.716 | 33.119 | 1.00 | 37.82 |
| ATOM | 7182 | N | VAL | 3316 | 52.745 | −15.262 | 31.627 | 1.00 | 37.55 |
| ATOM | 7183 | CA | VAL | 3316 | 52.560 | −16.560 | 30.975 | 1.00 | 38.82 |
| ATOM | 7184 | CB | VAL | 3316 | 53.410 | −16.717 | 29.692 | 1.00 | 39.14 |
| ATOM | 7185 | CG1 | VAL | 3316 | 53.239 | −18.125 | 29.142 | 1.00 | 38.87 |
| ATOM | 7186 | CG2 | VAL | 3316 | 52.972 | −15.705 | 28.645 | 1.00 | 39.46 |
| ATOM | 7187 | C | VAL | 3316 | 52.887 | −17.744 | 31.878 | 1.00 | 39.11 |
| ATOM | 7188 | O | VAL | 3316 | 52.374 | −18.835 | 31.660 | 1.00 | 39.40 |
| ATOM | 7189 | N | ASN | 3317 | 53.726 | −17.527 | 32.887 | 1.00 | 39.65 |
| ATOM | 7190 | CA | ASN | 3317 | 54.125 | −18.595 | 33.797 | 1.00 | 40.12 |
| ATOM | 7191 | CB | ASN | 3317 | 55.564 | −18.356 | 34.240 | 1.00 | 39.84 |
| ATOM | 7192 | CG | ASN | 3317 | 56.537 | −18.590 | 33.117 | 1.00 | 39.94 |
| ATOM | 7193 | OD1 | ASN | 3317 | 57.546 | −17.899 | 32.987 | 1.00 | 39.93 |
| ATOM | 7194 | ND2 | ASN | 3317 | 56.233 | −19.582 | 32.284 | 1.00 | 39.60 |
| ATOM | 7195 | C | ASN | 3317 | 53.201 | −18.741 | 34.998 | 1.00 | 40.98 |
| ATOM | 7196 | O | ASN | 3317 | 53.179 | −19.783 | 35.660 | 1.00 | 41.56 |
| ATOM | 7197 | N | THR | 3318 | 52.439 | −17.697 | 35.288 | 1.00 | 41.10 |
| ATOM | 7198 | CA | THR | 3318 | 51.496 | −17.755 | 36.394 | 1.00 | 41.08 |
| ATOM | 7199 | CB | THR | 3318 | 51.991 | −16.952 | 37.617 | 1.00 | 42.00 |
| ATOM | 7200 | OG1 | THR | 3318 | 53.406 | −17.138 | 37.776 | 1.00 | 42.41 |
| ATOM | 7201 | CG2 | THR | 3318 | 51.290 | −17.434 | 38.881 | 1.00 | 41.71 |
| ATOM | 7202 | C | THR | 3318 | 50.219 | −17.144 | 35.845 | 1.00 | 41.10 |
| ATOM | 7203 | O | THR | 3318 | 49.988 | −15.937 | 35.977 | 1.00 | 41.47 |
| ATOM | 7204 | N | THR | 3319 | 49.408 | −17.982 | 35.200 | 1.00 | 40.82 |
| ATOM | 7205 | CA | THR | 3319 | 48.156 | −17.530 | 34.596 | 1.00 | 40.88 |
| ATOM | 7206 | CB | THR | 3319 | 47.456 | −18.636 | 33.805 | 1.00 | 40.73 |
| ATOM | 7207 | OG1 | THR | 3319 | 47.059 | −19.677 | 34.699 | 1.00 | 41.93 |
| ATOM | 7208 | CG2 | THR | 3319 | 48.370 | −19.189 | 32.739 | 1.00 | 40.84 |
| ATOM | 7209 | C | THR | 3319 | 47.147 | −17.000 | 35.591 | 1.00 | 41.01 |
| ATOM | 7210 | O | THR | 3319 | 47.259 | −17.225 | 36.799 | 1.00 | 40.86 |
| ATOM | 7211 | N | ASP | 3320 | 46.147 | −16.298 | 35.066 | 1.00 | 41.89 |
| ATOM | 7212 | CA | ASP | 3320 | 45.115 | −15.708 | 35.900 | 1.00 | 42.97 |
| ATOM | 7213 | CB | ASP | 3320 | 44.185 | −14.839 | 35.059 | 1.00 | 44.13 |
| ATOM | 7214 | CG | ASP | 3320 | 44.918 | −13.685 | 34.386 | 1.00 | 45.65 |
| ATOM | 7215 | OD1 | ASP | 3320 | 45.907 | −13.182 | 34.974 | 1.00 | 45.96 |
| ATOM | 7216 | OD2 | ASP | 3320 | 44.499 | −13.270 | 33.279 | 1.00 | 46.51 |
| ATOM | 7217 | C | ASP | 3320 | 44.318 | −16.746 | 36.672 | 1.00 | 43.73 |
| ATOM | 7218 | O | ASP | 3320 | 43.772 | −16.445 | 37.730 | 1.00 | 43.65 |
| ATOM | 7219 | N | LYS | 3321 | 44.264 | −17.970 | 36.150 | 1.00 | 44.86 |
| ATOM | 7220 | CA | LYS | 3321 | 43.543 | −19.059 | 36.812 | 1.00 | 45.74 |
| ATOM | 7221 | CB | LYS | 3321 | 43.957 | −20.430 | 36.243 | 1.00 | 47.38 |
| ATOM | 7222 | CG | LYS | 3321 | 43.535 | −20.721 | 34.806 | 1.00 | 50.38 |
| ATOM | 7223 | CD | LYS | 3321 | 43.587 | −22.230 | 34.512 | 1.00 | 51.42 |
| ATOM | 7224 | CE | LYS | 3321 | 43.010 | −22.571 | 33.128 | 1.00 | 51.62 |
| ATOM | 7225 | NZ | LYS | 3321 | 43.873 | −22.093 | 32.006 | 1.00 | 51.59 |
| ATOM | 7226 | C | LYS | 3321 | 43.853 | −19.083 | 38.299 | 1.00 | 45.49 |
| ATOM | 7227 | O | LYS | 3321 | 42.998 | −19.422 | 39.118 | 1.00 | 45.37 |
| ATOM | 7228 | N | GLU | 3322 | 45.085 | −18.735 | 38.650 | 1.00 | 45.54 |
| ATOM | 7229 | CA | GLU | 3322 | 45.471 | −18.776 | 40.052 | 1.00 | 46.26 |
| ATOM | 7230 | CB | GLU | 3322 | 46.513 | −19.866 | 40.253 | 1.00 | 48.12 |
| ATOM | 7231 | CG | GLU | 3322 | 47.718 | −19.736 | 39.358 | 1.00 | 50.64 |
| ATOM | 7232 | CD | GLU | 3322 | 48.544 | −21.004 | 39.350 | 1.00 | 52.54 |
| ATOM | 7233 | OE1 | GLU | 3322 | 48.909 | −21.487 | 40.451 | 1.00 | 52.82 |
| ATOM | 7234 | OE2 | GLU | 3322 | 48.821 | −21.517 | 38.242 | 1.00 | 53.67 |
| ATOM | 7235 | C | GLU | 3322 | 45.955 | −17.479 | 40.698 | 1.00 | 45.70 |
| ATOM | 7236 | O | GLU | 3322 | 46.520 | −17.508 | 41.794 | 1.00 | 45.36 |
| ATOM | 7237 | N | MSE | 3323 | 45.724 | −16.342 | 40.049 | 1.00 | 44.60 |
| ATOM | 7238 | CA | MSE | 3323 | 46.166 | −15.085 | 40.628 | 1.00 | 43.45 |
| ATOM | 7239 | CB | MSE | 3323 | 46.449 | −14.061 | 39.535 | 1.00 | 43.98 |
| ATOM | 7240 | CG | MSE | 3323 | 47.711 | −14.352 | 38.761 | 1.00 | 44.67 |
| ATOM | 7241 | SE | MSE | 3323 | 49.113 | −14.547 | 39.850 | 1.00 | 44.93 |
| ATOM | 7242 | CE | MSE | 3323 | 49.484 | −12.856 | 40.255 | 1.00 | 43.91 |
| ATOM | 7243 | C | MSE | 3323 | 45.201 | −14.487 | 41.640 | 1.00 | 42.83 |
| ATOM | 7244 | O | MSE | 3323 | 45.569 | −13.574 | 42.370 | 1.00 | 42.79 |
| ATOM | 7245 | N | GLU | 3324 | 43.976 | −14.998 | 41.694 | 1.00 | 42.68 |
| ATOM | 7246 | CA | GLU | 3324 | 42.977 | −14.482 | 42.626 | 1.00 | 42.76 |
| ATOM | 7247 | CB | GLU | 3324 | 41.563 | −14.805 | 42.119 | 1.00 | 43.54 |
| ATOM | 7248 | CG | GLU | 3324 | 41.279 | −14.316 | 40.703 | 1.00 | 46.16 |
| ATOM | 7249 | CD | GLU | 3324 | 39.790 | −14.039 | 40.431 | 1.00 | 47.70 |
| ATOM | 7250 | OE1 | GLU | 3324 | 38.964 | −14.985 | 40.542 | 1.00 | 47.99 |
| ATOM | 7251 | OE2 | GLU | 3324 | 39.456 | −12.867 | 40.100 | 1.00 | 47.37 |
| ATOM | 7252 | C | GLU | 3324 | 43.134 | −15.040 | 44.045 | 1.00 | 42.56 |
| ATOM | 7253 | O | GLU | 3324 | 42.310 | −14.782 | 44.920 | 1.00 | 42.37 |
| ATOM | 7254 | N | VAL | 3325 | 44.196 | −15.799 | 44.280 | 1.00 | 42.83 |
| ATOM | 7255 | CA | VAL | 3325 | 44.415 | −16.393 | 45.592 | 1.00 | 43.10 |
| ATOM | 7256 | CB | VAL | 3325 | 43.895 | −17.834 | 45.640 | 1.00 | 43.36 |
| ATOM | 7257 | CG1 | VAL | 3325 | 44.790 | −18.729 | 44.778 | 1.00 | 43.60 |
| ATOM | 7258 | CG2 | VAL | 3325 | 43.876 | −18.333 | 47.071 | 1.00 | 43.88 |
| ATOM | 7259 | C | VAL | 3325 | 45.893 | −16.458 | 45.940 | 1.00 | 43.23 |
| ATOM | 7260 | O | VAL | 3325 | 46.721 | −16.764 | 45.087 | 1.00 | 43.66 |
| ATOM | 7261 | N | LEU | 3326 | 46.221 | −16.177 | 47.197 | 1.00 | 43.48 |
| ATOM | 7262 | CA | LEU | 3326 | 47.606 | −16.238 | 47.653 | 1.00 | 43.43 |
| ATOM | 7263 | CB | LEU | 3326 | 48.087 | −14.881 | 48.166 | 1.00 | 44.53 |
| ATOM | 7264 | CG | LEU | 3326 | 49.465 | −14.933 | 48.841 | 1.00 | 44.58 |
| ATOM | 7265 | CD1 | LEU | 3326 | 50.502 | −15.399 | 47.829 | 1.00 | 44.70 |
| ATOM | 7266 | CD2 | LEU | 3326 | 49.838 | −13.565 | 49.390 | 1.00 | 44.44 |
| ATOM | 7267 | C | LEU | 3326 | 47.670 | −17.245 | 48.788 | 1.00 | 43.47 |
| ATOM | 7268 | O | LEU | 3326 | 46.982 | −17.092 | 49.802 | 1.00 | 42.57 |
| ATOM | 7269 | N | HIS | 3327 | 48.494 | −18.275 | 48.609 | 1.00 | 44.13 |
| ATOM | 7270 | CA | HIS | 3327 | 48.647 | −19.331 | 49.610 | 1.00 | 44.76 |
| ATOM | 7271 | CB | HIS | 3327 | 48.753 | −20.712 | 48.944 | 1.00 | 46.04 |
| ATOM | 7272 | CG | HIS | 3327 | 47.512 | −21.161 | 48.241 | 1.00 | 47.50 |
| ATOM | 7273 | CD2 | HIS | 3327 | 47.261 | −21.372 | 46.926 | 1.00 | 48.48 |
| ATOM | 7274 | ND1 | HIS | 3327 | 46.353 | −21.495 | 48.910 | 1.00 | 48.76 |
| ATOM | 7275 | CE1 | HIS | 3327 | 45.441 | −21.896 | 48.040 | 1.00 | 48.73 |
| ATOM | 7276 | NE2 | HIS | 3327 | 45.967 | −21.831 | 46.829 | 1.00 | 49.56 |
| ATOM | 7277 | C | HIS | 3327 | 49.904 | −19.141 | 50.441 | 1.00 | 44.48 |
| ATOM | 7278 | O | HIS | 3327 | 50.992 | −18.929 | 49.896 | 1.00 | 43.81 |
| ATOM | 7279 | N | LEU | 3328 | 49.747 | −19.228 | 51.755 | 1.00 | 44.48 |
| ATOM | 7280 | CA | LEU | 3328 | 50.873 | −19.125 | 52.675 | 1.00 | 45.32 |
| ATOM | 7281 | CB | LEU | 3328 | 50.734 | −17.891 | 53.569 | 1.00 | 44.54 |
| ATOM | 7282 | CG | LEU | 3328 | 50.711 | −16.517 | 52.897 | 1.00 | 44.09 |
| ATOM | 7283 | CD1 | LEU | 3328 | 50.605 | −15.449 | 53.957 | 1.00 | 44.36 |
| ATOM | 7284 | CD2 | LEU | 3328 | 51.962 | −16.299 | 52.088 | 1.00 | 43.15 |
| ATOM | 7285 | C | LEU | 3328 | 50.831 | −20.399 | 53.519 | 1.00 | 46.36 |
| ATOM | 7286 | O | LEU | 3328 | 49.836 | −20.669 | 54.200 | 1.00 | 46.50 |
| ATOM | 7287 | N | ARG | 3329 | 51.901 | −21.189 | 53.467 | 1.00 | 47.48 |
| ATOM | 7288 | CA | ARG | 3329 | 51.950 | −22.439 | 54.217 | 1.00 | 48.66 |
| ATOM | 7289 | CB | ARG | 3329 | 52.419 | −23.576 | 53.300 | 1.00 | 48.62 |
| ATOM | 7290 | C | ARG | 3329 | 52.837 | −22.362 | 55.456 | 1.00 | 49.00 |
| ATOM | 7291 | O | ARG | 3329 | 53.873 | −21.697 | 55.447 | 1.00 | 49.17 |
| ATOM | 7292 | N | ASN | 3330 | 52.412 | −23.042 | 56.519 | 1.00 | 50.00 |
| ATOM | 7293 | CA | ASN | 3330 | 53.165 | −23.077 | 57.777 | 1.00 | 50.87 |
| ATOM | 7294 | CB | ASN | 3330 | 54.329 | −24.072 | 57.650 | 1.00 | 52.14 |
| ATOM | 7295 | CG | ASN | 3330 | 55.248 | −24.070 | 58.865 | 1.00 | 53.47 |
| ATOM | 7296 | OD1 | ASN | 3330 | 56.414 | −24.471 | 58.774 | 1.00 | 53.46 |
| ATOM | 7297 | ND2 | ASN | 3330 | 54.728 | −23.626 | 60.010 | 1.00 | 54.22 |
| ATOM | 7298 | C | ASN | 3330 | 53.711 | −21.684 | 58.081 | 1.00 | 50.75 |
| ATOM | 7299 | O | ASN | 3330 | 54.925 | −21.455 | 58.023 | 1.00 | 50.13 |
| ATOM | 7300 | N | VAL | 3331 | 52.815 | −20.756 | 58.406 | 1.00 | 50.65 |
| ATOM | 7301 | CA | VAL | 3331 | 53.231 | −19.388 | 58.674 | 1.00 | 51.07 |
| ATOM | 7302 | CB | VAL | 3331 | 52.020 | −18.423 | 58.724 | 1.00 | 51.24 |
| ATOM | 7303 | CG1 | VAL | 3331 | 51.394 | −18.315 | 57.351 | 1.00 | 51.38 |
| ATOM | 7304 | CG2 | VAL | 3331 | 50.995 | −18.911 | 59.738 | 1.00 | 51.49 |
| ATOM | 7305 | C | VAL | 3331 | 54.047 | −19.200 | 59.942 | 1.00 | 51.22 |
| ATOM | 7306 | O | VAL | 3331 | 53.802 | −19.827 | 60.967 | 1.00 | 51.28 |
| ATOM | 7307 | N | SER | 3332 | 55.035 | −18.323 | 59.849 | 1.00 | 51.54 |
| ATOM | 7308 | CA | SER | 3332 | 55.897 | −18.001 | 60.970 | 1.00 | 51.95 |
| ATOM | 7309 | CB | SER | 3332 | 57.350 | −17.992 | 60.506 | 1.00 | 52.28 |
| ATOM | 7310 | OG | SER | 3332 | 58.078 | −16.956 | 61.146 | 1.00 | 52.72 |
| ATOM | 7311 | C | SER | 3332 | 55.502 | −16.609 | 61.456 | 1.00 | 52.10 |
| ATOM | 7312 | O | SER | 3332 | 54.559 | −16.019 | 60.940 | 1.00 | 52.43 |
| ATOM | 7313 | N | PHE | 3333 | 56.215 | −16.085 | 62.447 | 1.00 | 52.56 |
| ATOM | 7314 | CA | PHE | 3333 | 55.934 | −14.743 | 62.951 | 1.00 | 52.86 |
| ATOM | 7315 | CB | PHE | 3333 | 56.681 | −14.480 | 64.261 | 1.00 | 52.88 |
| ATOM | 7316 | CG | PHE | 3333 | 55.926 | −14.894 | 65.480 | 1.00 | 53.46 |
| ATOM | 7317 | CD1 | PHE | 3333 | 56.428 | −14.622 | 66.740 | 1.00 | 54.44 |
| ATOM | 7318 | CD2 | PHE | 3333 | 54.703 | −15.546 | 65.372 | 1.00 | 54.26 |
| ATOM | 7319 | CE1 | PHE | 3333 | 55.722 | −14.994 | 67.881 | 1.00 | 55.87 |
| ATOM | 7320 | CE2 | PHE | 3333 | 53.987 | −15.923 | 66.503 | 1.00 | 55.10 |
| ATOM | 7321 | CZ | PHE | 3333 | 54.494 | −15.649 | 67.758 | 1.00 | 55.49 |
| ATOM | 7322 | C | PHE | 3333 | 56.396 | −13.732 | 61.919 | 1.00 | 52.78 |
| ATOM | 7323 | O | PHE | 3333 | 55.937 | −12.598 | 61.889 | 1.00 | 53.08 |
| ATOM | 7324 | N | GLU | 3334 | 57.328 | −14.160 | 61.082 | 1.00 | 53.19 |
| ATOM | 7325 | CA | GLU | 3334 | 57.875 | −13.314 | 60.037 | 1.00 | 53.59 |
| ATOM | 7326 | CB | GLU | 3334 | 59.041 | −14.025 | 59.352 | 1.00 | 55.16 |
| ATOM | 7327 | CG | GLU | 3334 | 60.125 | −14.514 | 60.291 | 1.00 | 58.14 |
| ATOM | 7328 | CD | GLU | 3334 | 60.869 | −15.715 | 59.723 | 1.00 | 60.04 |
| ATOM | 7329 | OE1 | GLU | 3334 | 61.934 | −16.085 | 60.279 | 1.00 | 60.47 |
| ATOM | 7330 | OE2 | GLU | 3334 | 60.375 | −16.294 | 58.722 | 1.00 | 60.93 |
| ATOM | 7331 | C | GLU | 3334 | 56.802 | −13.037 | 58.997 | 1.00 | 52.81 |
| ATOM | 7332 | O | GLU | 3334 | 56.747 | −11.951 | 58.416 | 1.00 | 53.24 |
| ATOM | 7333 | N | ASP | 3335 | 55.955 | −14.033 | 58.762 | 1.00 | 51.46 |
| ATOM | 7334 | CA | ASP | 3335 | 54.895 | −13.916 | 57.777 | 1.00 | 49.82 |
| ATOM | 7335 | CB | ASP | 3335 | 54.249 | −15.283 | 57.540 | 1.00 | 51.63 |
| ATOM | 7336 | CG | ASP | 3335 | 55.216 | −16.289 | 56.932 | 1.00 | 53.20 |
| ATOM | 7337 | OD1 | ASP | 3335 | 55.917 | −15.919 | 55.963 | 1.00 | 53.59 |
| ATOM | 7338 | OD2 | ASP | 3335 | 55.265 | −17.447 | 57.417 | 1.00 | 54.31 |
| ATOM | 7339 | C | ASP | 3335 | 53.823 | −12.895 | 58.145 | 1.00 | 48.09 |
| ATOM | 7340 | O | ASP | 3335 | 53.102 | −12.404 | 57.275 | 1.00 | 47.80 |
| ATOM | 7341 | N | ALA | 3336 | 53.709 | −12.575 | 59.428 | 1.00 | 45.74 |
| ATOM | 7342 | CA | ALA | 3336 | 52.708 | −11.600 | 59.858 | 1.00 | 43.81 |
| ATOM | 7343 | CB | ALA | 3336 | 52.749 | −11.432 | 61.380 | 1.00 | 42.82 |
| ATOM | 7344 | C | ALA | 3336 | 52.992 | −10.265 | 59.167 | 1.00 | 42.18 |
| ATOM | 7345 | O | ALA | 3336 | 54.139 | −9.984 | 58.804 | 1.00 | 42.16 |
| ATOM | 7346 | N | GLY | 3337 | 51.953 | −9.453 | 58.981 | 1.00 | 40.23 |
| ATOM | 7347 | CA | GLY | 3337 | 52.139 | −8.169 | 58.334 | 1.00 | 38.90 |
| ATOM | 7348 | C | GLY | 3337 | 51.065 | −7.814 | 57.321 | 1.00 | 37.99 |
| ATOM | 7349 | O | GLY | 3337 | 50.007 | −8.450 | 57.268 | 1.00 | 37.35 |
| ATOM | 7350 | N | GLU | 3338 | 51.344 | −6.805 | 56.500 | 1.00 | 36.93 |
| ATOM | 7351 | CA | GLU | 3338 | 50.373 | −6.360 | 55.515 | 1.00 | 36.36 |
| ATOM | 7352 | CB | GLU | 3338 | 50.333 | −4.824 | 55.504 | 1.00 | 37.46 |
| ATOM | 7353 | CG | GLU | 3338 | 49.248 | −4.213 | 54.629 | 1.00 | 38.69 |
| ATOM | 7354 | CD | GLU | 3338 | 49.175 | −2.698 | 54.762 | 1.00 | 39.92 |
| ATOM | 7355 | OE1 | GLU | 3338 | 50.247 | −2.039 | 54.748 | 1.00 | 39.95 |
| ATOM | 7356 | OE2 | GLU | 3338 | 48.043 | −2.165 | 54.869 | 1.00 | 40.48 |
| ATOM | 7357 | C | GLU | 3338 | 50.607 | −6.916 | 54.114 | 1.00 | 35.44 |
| ATOM | 7358 | O | GLU | 3338 | 51.713 | −6.854 | 53.572 | 1.00 | 35.82 |
| ATOM | 7359 | N | TYR | 3339 | 49.549 | −7.472 | 53.541 | 1.00 | 34.38 |
| ATOM | 7360 | CA | TYR | 3339 | 49.602 | −8.046 | 52.204 | 1.00 | 34.33 |
| ATOM | 7361 | CB | TYR | 3339 | 49.139 | −9.500 | 52.230 | 1.00 | 35.86 |
| ATOM | 7362 | CG | TYR | 3339 | 50.087 | −10.385 | 52.984 | 1.00 | 37.43 |
| ATOM | 7363 | CD1 | TYR | 3339 | 50.207 | −10.294 | 54.377 | 1.00 | 38.20 |
| ATOM | 7364 | CE1 | TYR | 3339 | 51.122 | −11.084 | 55.076 | 1.00 | 38.38 |
| ATOM | 7365 | CD2 | TYR | 3339 | 50.902 | −11.287 | 52.306 | 1.00 | 38.51 |
| ATOM | 7366 | CE2 | TYR | 3339 | 51.821 | −12.079 | 52.992 | 1.00 | 39.35 |
| ATOM | 7367 | CZ | TYR | 3339 | 51.924 | −11.972 | 54.374 | 1.00 | 38.70 |
| ATOM | 7368 | OH | TYR | 3339 | 52.827 | −12.766 | 55.031 | 1.00 | 38.90 |
| ATOM | 7369 | C | TYR | 3339 | 48.713 | −7.246 | 51.286 | 1.00 | 33.35 |
| ATOM | 7370 | O | TYR | 3339 | 47.612 | −6.843 | 51.671 | 1.00 | 33.18 |
| ATOM | 7371 | N | THR | 3340 | 49.185 | −7.016 | 50.068 | 1.00 | 32.84 |
| ATOM | 7372 | CA | THR | 3340 | 48.406 | −6.235 | 49.118 | 1.00 | 32.60 |
| ATOM | 7373 | CB | THR | 3340 | 49.060 | −4.830 | 48.872 | 1.00 | 32.53 |
| ATOM | 7374 | OG1 | THR | 3340 | 48.902 | −4.023 | 50.040 | 1.00 | 33.01 |
| ATOM | 7375 | CG2 | THR | 3340 | 48.403 | −4.098 | 47.696 | 1.00 | 32.70 |
| ATOM | 7376 | C | THR | 3340 | 48.174 | −6.912 | 47.779 | 1.00 | 32.25 |
| ATOM | 7377 | O | THR | 3340 | 49.047 | −7.583 | 47.232 | 1.00 | 31.96 |
| ATOM | 7378 | N | CYS | 3341 | 46.965 | −6.729 | 47.267 | 1.00 | 33.05 |
| ATOM | 7379 | CA | CYS | 3341 | 46.597 | −7.248 | 45.964 | 1.00 | 33.97 |
| ATOM | 7380 | CB | CYS | 3341 | 45.227 | −7.900 | 46.003 | 1.00 | 34.97 |
| ATOM | 7381 | SG | CYS | 3341 | 44.857 | −8.553 | 44.389 | 1.00 | 40.69 |
| ATOM | 7382 | C | CYS | 3341 | 46.558 | −6.037 | 45.035 | 1.00 | 32.96 |
| ATOM | 7383 | O | CYS | 3341 | 45.682 | −5.183 | 45.158 | 1.00 | 32.50 |
| ATOM | 7384 | N | LEU | 3342 | 47.513 | −5.971 | 44.116 | 1.00 | 32.48 |
| ATOM | 7385 | CA | LEU | 3342 | 47.616 | −4.858 | 43.189 | 1.00 | 32.48 |
| ATOM | 7386 | CB | LEU | 3342 | 49.076 | −4.407 | 43.135 | 1.00 | 33.15 |
| ATOM | 7387 | CG | LEU | 3342 | 49.454 | −3.104 | 42.430 | 1.00 | 34.51 |
| ATOM | 7388 | CD1 | LEU | 3342 | 49.092 | −1.912 | 43.303 | 1.00 | 34.35 |
| ATOM | 7389 | CD2 | LEU | 3342 | 50.952 | −3.099 | 42.176 | 1.00 | 35.12 |
| ATOM | 7390 | C | LEU | 3342 | 47.134 | −5.263 | 41.791 | 1.00 | 32.09 |
| ATOM | 7391 | O | LEU | 3342 | 47.558 | −6.283 | 41.253 | 1.00 | 33.01 |
| ATOM | 7392 | N | ALA | 3343 | 46.250 | −4.469 | 41.200 | 1.00 | 31.02 |
| ATOM | 7393 | CA | ALA | 3343 | 45.739 | −4.784 | 39.868 | 1.00 | 30.21 |
| ATOM | 7394 | CB | ALA | 3343 | 44.328 | −5.375 | 39.966 | 1.00 | 29.15 |
| ATOM | 7395 | C | ALA | 3343 | 45.717 | −3.541 | 38.997 | 1.00 | 30.06 |
| ATOM | 7396 | O | ALA | 3343 | 45.231 | −2.489 | 39.409 | 1.00 | 31.07 |
| ATOM | 7397 | N | GLY | 3344 | 46.242 | −3.648 | 37.786 | 1.00 | 29.35 |
| ATOM | 7398 | CA | GLY | 3344 | 46.219 | −2.491 | 36.918 | 1.00 | 29.15 |
| ATOM | 7399 | C | GLY | 3344 | 46.026 | −2.823 | 35.456 | 1.00 | 29.17 |
| ATOM | 7400 | O | GLY | 3344 | 46.191 | −3.971 | 35.033 | 1.00 | 29.56 |
| ATOM | 7401 | N | ASN | 3345 | 45.631 | −1.813 | 34.690 | 1.00 | 28.73 |
| ATOM | 7402 | CA | ASN | 3345 | 45.459 | −1.944 | 33.250 | 1.00 | 28.57 |
| ATOM | 7403 | CB | ASN | 3345 | 44.006 | −2.248 | 32.859 | 1.00 | 28.07 |
| ATOM | 7404 | CG | ASN | 3345 | 43.018 | −1.228 | 33.377 | 1.00 | 29.42 |
| ATOM | 7405 | OD1 | ASN | 3345 | 43.270 | −0.018 | 33.356 | 1.00 | 29.45 |
| ATOM | 7406 | ND2 | ASN | 3345 | 41.863 | −1.712 | 33.827 | 1.00 | 29.23 |
| ATOM | 7407 | C | ASN | 3345 | 45.933 | −0.615 | 32.672 | 1.00 | 29.54 |
| ATOM | 7408 | O | ASN | 3345 | 46.382 | 0.257 | 33.420 | 1.00 | 29.62 |
| ATOM | 7409 | N | SER | 3346 | 45.851 | −0.442 | 31.359 | 1.00 | 30.06 |
| ATOM | 7410 | CA | SER | 3346 | 46.340 | 0.797 | 30.758 | 1.00 | 30.55 |
| ATOM | 7411 | CB | SER | 3346 | 46.133 | 0.781 | 29.235 | 1.00 | 30.53 |
| ATOM | 7412 | OG | SER | 3346 | 45.062 | 1.619 | 28.836 | 1.00 | 31.68 |
| ATOM | 7413 | C | SER | 3346 | 45.714 | 2.047 | 31.364 | 1.00 | 31.09 |
| ATOM | 7414 | O | SER | 3346 | 46.365 | 3.087 | 31.450 | 1.00 | 32.05 |
| ATOM | 7415 | N | ILE | 3347 | 44.466 | 1.944 | 31.811 | 1.00 | 31.38 |
| ATOM | 7416 | CA | ILE | 3347 | 43.765 | 3.089 | 32.399 | 1.00 | 31.08 |
| ATOM | 7417 | CB | ILE | 3347 | 42.232 | 2.891 | 32.326 | 1.00 | 30.92 |
| ATOM | 7418 | CG2 | ILE | 3347 | 41.524 | 4.096 | 32.941 | 1.00 | 29.41 |
| ATOM | 7419 | CG1 | ILE | 3347 | 41.816 | 2.688 | 30.862 | 1.00 | 29.84 |
| ATOM | 7420 | CD1 | ILE | 3347 | 40.474 | 2.016 | 30.663 | 1.00 | 29.21 |
| ATOM | 7421 | C | ILE | 3347 | 44.136 | 3.454 | 33.839 | 1.00 | 31.37 |
| ATOM | 7422 | O | ILE | 3347 | 44.069 | 4.630 | 34.206 | 1.00 | 31.86 |
| ATOM | 7423 | N | GLY | 3348 | 44.518 | 2.474 | 34.656 | 1.00 | 31.56 |
| ATOM | 7424 | CA | GLY | 3348 | 44.874 | 2.785 | 36.034 | 1.00 | 31.39 |
| ATOM | 7425 | C | GLY | 3348 | 45.158 | 1.612 | 36.964 | 1.00 | 32.13 |
| ATOM | 7426 | O | GLY | 3348 | 45.062 | 0.440 | 36.570 | 1.00 | 32.10 |
| ATOM | 7427 | N | LEU | 3349 | 45.510 | 1.933 | 38.211 | 1.00 | 32.34 |
| ATOM | 7428 | CA | LEU | 3349 | 45.815 | 0.924 | 39.228 | 1.00 | 32.22 |
| ATOM | 7429 | CB | LEU | 3349 | 47.237 | 1.091 | 39.766 | 1.00 | 32.93 |
| ATOM | 7430 | CG | LEU | 3349 | 48.377 | 0.584 | 38.891 | 1.00 | 34.83 |
| ATOM | 7431 | CD1 | LEU | 3349 | 48.439 | 1.372 | 37.595 | 1.00 | 35.45 |
| ATOM | 7432 | CD2 | LEU | 3349 | 49.679 | 0.730 | 39.650 | 1.00 | 35.87 |
| ATOM | 7433 | C | LEU | 3349 | 44.851 | 0.911 | 40.412 | 1.00 | 31.88 |
| ATOM | 7434 | O | LEU | 3349 | 44.263 | 1.925 | 40.783 | 1.00 | 31.99 |
| ATOM | 7435 | N | SER | 3350 | 44.701 | −0.258 | 41.011 | 1.00 | 31.38 |
| ATOM | 7436 | CA | SER | 3350 | 43.827 | −0.410 | 42.150 | 1.00 | 31.50 |
| ATOM | 7437 | CB | SER | 3350 | 42.454 | −0.908 | 41.709 | 1.00 | 33.26 |
| ATOM | 7438 | OG | SER | 3350 | 41.766 | 0.076 | 40.951 | 1.00 | 35.68 |
| ATOM | 7439 | C | SER | 3350 | 44.466 | −1.421 | 43.078 | 1.00 | 31.48 |
| ATOM | 7440 | O | SER | 3350 | 45.238 | −2.273 | 42.641 | 1.00 | 30.86 |
| ATOM | 7441 | N | HIS | 3351 | 44.158 | −1.322 | 44.365 | 1.00 | 31.34 |
| ATOM | 7442 | CA | HIS | 3351 | 44.721 | −2.253 | 45.313 | 1.00 | 31.29 |
| ATOM | 7443 | CB | HIS | 3351 | 46.179 | −1.891 | 45.598 | 1.00 | 32.94 |
| ATOM | 7444 | CG | HIS | 3351 | 46.344 | −0.621 | 46.369 | 1.00 | 35.14 |
| ATOM | 7445 | CD2 | HIS | 3351 | 46.357 | −0.386 | 47.704 | 1.00 | 35.09 |
| ATOM | 7446 | ND1 | HIS | 3351 | 46.488 | 0.608 | 45.760 | 1.00 | 36.43 |
| ATOM | 7447 | CE1 | HIS | 3351 | 46.584 | 1.545 | 46.689 | 1.00 | 36.63 |
| ATOM | 7448 | NE2 | HIS | 3351 | 46.508 | 0.967 | 47.876 | 1.00 | 35.77 |
| ATOM | 7449 | C | HIS | 3351 | 43.939 | −2.302 | 46.608 | 1.00 | 30.98 |
| ATOM | 7450 | O | HIS | 3351 | 43.307 | −1.326 | 47.006 | 1.00 | 31.38 |
| ATOM | 7451 | N | HIS | 3352 | 43.977 | −3.463 | 47.247 | 1.00 | 30.50 |
| ATOM | 7452 | CA | HIS | 3352 | 43.310 | −3.690 | 48.521 | 1.00 | 31.11 |
| ATOM | 7453 | CB | HIS | 3352 | 42.134 | −4.667 | 48.368 | 1.00 | 32.61 |
| ATOM | 7454 | CG | HIS | 3352 | 40.832 | −4.022 | 47.997 | 1.00 | 34.73 |
| ATOM | 7455 | CD2 | HIS | 3352 | 40.529 | −2.749 | 47.641 | 1.00 | 35.31 |
| ATOM | 7456 | ND1 | HIS | 3352 | 39.643 | −4.721 | 47.967 | 1.00 | 34.42 |
| ATOM | 7457 | CE1 | HIS | 3352 | 38.664 | −3.907 | 47.609 | 1.00 | 35.07 |
| ATOM | 7458 | NE2 | HIS | 3352 | 39.175 | −2.704 | 47.406 | 1.00 | 35.61 |
| ATOM | 7459 | C | HIS | 3352 | 44.373 | −4.323 | 49.409 | 1.00 | 30.95 |
| ATOM | 7460 | O | HIS | 3352 | 45.243 | −5.050 | 48.922 | 1.00 | 31.37 |
| ATOM | 7461 | N | SER | 3353 | 44.316 | −4.049 | 50.704 | 1.00 | 30.04 |
| ATOM | 7462 | CA | SER | 3353 | 45.297 | −4.614 | 51.612 | 1.00 | 29.83 |
| ATOM | 7463 | CB | SER | 3353 | 46.247 | −3.527 | 52.106 | 1.00 | 29.77 |
| ATOM | 7464 | OG | SER | 3353 | 46.735 | −2.761 | 51.022 | 1.00 | 31.77 |
| ATOM | 7465 | C | SER | 3353 | 44.618 | −5.270 | 52.791 | 1.00 | 29.79 |
| ATOM | 7466 | O | SER | 3353 | 43.494 | −4.921 | 53.160 | 1.00 | 28.89 |
| ATOM | 7467 | N | ALA | 3354 | 45.300 | −6.237 | 53.379 | 1.00 | 29.91 |
| ATOM | 7468 | CA | ALA | 3354 | 44.750 | −6.907 | 54.537 | 1.00 | 31.47 |
| ATOM | 7469 | CB | ALA | 3354 | 44.032 | −8.204 | 54.131 | 1.00 | 30.88 |
| ATOM | 7470 | C | ALA | 3354 | 45.911 | −7.188 | 55.467 | 1.00 | 32.16 |
| ATOM | 7471 | O | ALA | 3354 | 47.067 | −7.197 | 55.044 | 1.00 | 31.55 |
| ATOM | 7472 | N | TRP | 3355 | 45.605 | −7.384 | 56.741 | 1.00 | 33.27 |
| ATOM | 7473 | CA | TRP | 3355 | 46.650 | −7.658 | 57.692 | 1.00 | 34.86 |
| ATOM | 7474 | CB | TRP | 3355 | 46.568 | −6.689 | 58.862 | 1.00 | 37.11 |
| ATOM | 7475 | CG | TRP | 3355 | 47.873 | −6.595 | 59.565 | 1.00 | 40.21 |
| ATOM | 7476 | CD2 | TRP | 3355 | 48.796 | −5.508 | 59.487 | 1.00 | 41.19 |
| ATOM | 7477 | CE2 | TRP | 3355 | 49.950 | −5.888 | 60.218 | 1.00 | 42.23 |
| ATOM | 7478 | CE3 | TRP | 3355 | 48.763 | −4.251 | 58.868 | 1.00 | 41.95 |
| ATOM | 7479 | CD1 | TRP | 3355 | 48.481 | −7.567 | 60.329 | 1.00 | 40.96 |
| ATOM | 7480 | NE1 | TRP | 3355 | 49.732 | −7.146 | 60.721 | 1.00 | 41.71 |
| ATOM | 7481 | CZ2 | TRP | 3355 | 51.060 | −5.050 | 60.345 | 1.00 | 42.59 |
| ATOM | 7482 | CZ3 | TRP | 3355 | 49.864 | −3.420 | 58.994 | 1.00 | 42.71 |
| ATOM | 7483 | CH2 | TRP | 3355 | 51.000 | −3.823 | 59.729 | 1.00 | 43.09 |
| ATOM | 7484 | C | TRP | 3355 | 46.584 | −9.095 | 58.193 | 1.00 | 35.22 |
| ATOM | 7485 | O | TRP | 3355 | 45.504 | −9.633 | 58.446 | 1.00 | 34.37 |
| ATOM | 7486 | N | LEU | 3356 | 47.751 | −9.716 | 58.321 | 1.00 | 35.39 |
| ATOM | 7487 | CA | LEU | 3356 | 47.829 | −11.084 | 58.794 | 1.00 | 35.91 |
| ATOM | 7488 | CB | LEU | 3356 | 48.700 | −11.908 | 57.845 | 1.00 | 35.85 |
| ATOM | 7489 | CG | LEU | 3356 | 48.772 | −13.439 | 57.935 | 1.00 | 35.87 |
| ATOM | 7490 | CD1 | LEU | 3356 | 50.144 | −13.848 | 58.418 | 1.00 | 36.03 |
| ATOM | 7491 | CD2 | LEU | 3356 | 47.673 | −13.986 | 58.832 | 1.00 | 35.53 |
| ATOM | 7492 | C | LEU | 3356 | 48.415 | −11.076 | 60.201 | 1.00 | 36.68 |
| ATOM | 7493 | O | LEU | 3356 | 49.530 | −10.610 | 60.415 | 1.00 | 36.79 |
| ATOM | 7494 | N | THR | 3357 | 47.642 | −11.576 | 61.162 | 1.00 | 37.27 |
| ATOM | 7495 | CA | THR | 3357 | 48.063 | −11.634 | 62.556 | 1.00 | 37.55 |
| ATOM | 7496 | CB | THR | 3357 | 46.933 | −11.180 | 63.490 | 1.00 | 37.49 |
| ATOM | 7497 | OG1 | THR | 3357 | 46.447 | −9.897 | 63.079 | 1.00 | 36.40 |
| ATOM | 7498 | CG2 | THR | 3357 | 47.431 | −11.104 | 64.919 | 1.00 | 37.98 |
| ATOM | 7499 | C | THR | 3357 | 48.406 | −13.082 | 62.911 | 1.00 | 38.40 |
| ATOM | 7500 | O | THR | 3357 | 47.574 | −13.977 | 62.771 | 1.00 | 38.20 |
| ATOM | 7501 | N | VAL | 3358 | 49.626 | −13.304 | 63.380 | 1.00 | 39.41 |
| ATOM | 7502 | CA | VAL | 3358 | 50.082 | −14.640 | 63.741 | 1.00 | 40.99 |
| ATOM | 7503 | CB | VAL | 3358 | 51.438 | −14.941 | 63.046 | 1.00 | 40.75 |
| ATOM | 7504 | CG1 | VAL | 3358 | 51.897 | −16.352 | 63.365 | 1.00 | 41.08 |
| ATOM | 7505 | CG2 | VAL | 3358 | 51.305 | −14.743 | 61.540 | 1.00 | 40.17 |
| ATOM | 7506 | C | VAL | 3358 | 50.256 | −14.795 | 65.254 | 1.00 | 41.93 |
| ATOM | 7507 | O | VAL | 3358 | 50.966 | −14.016 | 65.876 | 1.00 | 42.39 |
| ATOM | 7508 | N | LEU | 3359 | 49.605 | −15.800 | 65.837 | 1.00 | 43.17 |
| ATOM | 7509 | CA | LEU | 3359 | 49.710 | −16.066 | 67.274 | 1.00 | 44.22 |
| ATOM | 7510 | CB | LEU | 3359 | 48.324 | −16.129 | 67.914 | 1.00 | 44.18 |
| ATOM | 7511 | CG | LEU | 3359 | 47.327 | −15.016 | 67.583 | 1.00 | 44.86 |
| ATOM | 7512 | CD1 | LEU | 3359 | 46.035 | −15.270 | 68.346 | 1.00 | 45.20 |
| ATOM | 7513 | CD2 | LEU | 3359 | 47.886 | −13.663 | 67.950 | 1.00 | 45.39 |
| ATOM | 7514 | C | LEU | 3359 | 50.417 | −17.406 | 67.498 | 1.00 | 44.51 |
| ATOM | 7515 | O | LEU | 3359 | 50.826 | −17.656 | 68.655 | 1.00 | 45.83 |
| ATOM | 7516 | CB | MSE | 4149 | 55.752 | −7.531 | 106.532 | 1.00 | 71.20 |
| ATOM | 7517 | CG | MSE | 4149 | 54.736 | −8.642 | 106.214 | 1.00 | 72.56 |
| ATOM | 7518 | SE | MSE | 4149 | 55.067 | −10.319 | 106.853 | 1.00 | 74.40 |
| ATOM | 7519 | CE | MSE | 4149 | 54.131 | −10.289 | 108.404 | 1.00 | 73.33 |
| ATOM | 7520 | C | MSE | 4149 | 56.974 | −8.057 | 104.391 | 1.00 | 69.48 |
| ATOM | 7521 | O | MSE | 4149 | 56.002 | −7.677 | 103.739 | 1.00 | 69.80 |
| ATOM | 7522 | N | MSE | 4149 | 57.883 | −6.381 | 105.972 | 1.00 | 70.34 |
| ATOM | 7523 | CA | MSE | 4149 | 57.131 | −7.667 | 105.862 | 1.00 | 70.46 |
| ATOM | 7524 | N | PRO | 4150 | 57.945 | −8.812 | 103.848 | 1.00 | 68.10 |
| ATOM | 7525 | CD | PRO | 4150 | 59.230 | −9.111 | 104.503 | 1.00 | 67.49 |
| ATOM | 7526 | CA | PRO | 4150 | 57.964 | −9.281 | 102.458 | 1.00 | 66.67 |
| ATOM | 7527 | CB | PRO | 4150 | 59.220 | −10.142 | 102.408 | 1.00 | 66.76 |
| ATOM | 7528 | CG | PRO | 4150 | 60.134 | −9.407 | 103.323 | 1.00 | 67.00 |
| ATOM | 7529 | C | PRO | 4150 | 56.720 | −10.042 | 102.012 | 1.00 | 65.11 |
| ATOM | 7530 | O | PRO | 4150 | 56.303 | −11.010 | 102.649 | 1.00 | 64.77 |
| ATOM | 7531 | N | VAL | 4151 | 56.144 | −9.593 | 100.900 | 1.00 | 63.58 |
| ATOM | 7532 | CA | VAL | 4151 | 54.945 | −10.202 | 100.332 | 1.00 | 61.56 |
| ATOM | 7533 | CB | VAL | 4151 | 53.703 | −9.317 | 100.572 | 1.00 | 61.21 |
| ATOM | 7534 | CG1 | VAL | 4151 | 52.448 | −10.078 | 100.201 | 1.00 | 60.49 |
| ATOM | 7535 | CG2 | VAL | 4151 | 53.650 | −8.866 | 102.020 | 1.00 | 61.29 |
| ATOM | 7536 | C | VAL | 4151 | 55.095 | −10.384 | 98.823 | 1.00 | 60.33 |
| ATOM | 7537 | O | VAL | 4151 | 55.314 | −9.414 | 98.094 | 1.00 | 60.08 |
| ATOM | 7538 | N | ALA | 4152 | 54.979 | −11.624 | 98.355 | 1.00 | 58.62 |
| ATOM | 7539 | CA | ALA | 4152 | 55.085 | −11.899 | 96.926 | 1.00 | 56.87 |
| ATOM | 7540 | CB | ALA | 4152 | 55.167 | −13.391 | 96.676 | 1.00 | 56.65 |
| ATOM | 7541 | C | ALA | 4152 | 53.851 | −11.307 | 96.245 | 1.00 | 56.09 |
| ATOM | 7542 | O | ALA | 4152 | 52.743 | −11.351 | 96.794 | 1.00 | 56.10 |
| ATOM | 7543 | N | PRO | 4153 | 54.024 | −10.751 | 95.033 | 1.00 | 54.53 |
| ATOM | 7544 | CD | PRO | 4153 | 55.249 | −10.779 | 94.221 | 1.00 | 54.03 |
| ATOM | 7545 | CA | PRO | 4153 | 52.926 | −10.138 | 94.281 | 1.00 | 52.98 |
| ATOM | 7546 | CB | PRO | 4153 | 53.565 | −9.829 | 92.924 | 1.00 | 52.76 |
| ATOM | 7547 | CG | PRO | 4153 | 54.692 | −10.783 | 92.835 | 1.00 | 53.64 |
| ATOM | 7548 | C | PRO | 4153 | 51.621 | −10.919 | 94.166 | 1.00 | 51.43 |
| ATOM | 7549 | O | PRO | 4153 | 51.612 | −12.132 | 94.020 | 1.00 | 51.48 |
| ATOM | 7550 | N | TYR | 4154 | 50.513 | −10.196 | 94.256 | 1.00 | 50.01 |
| ATOM | 7551 | CA | TYR | 4154 | 49.189 | −10.792 | 94.143 | 1.00 | 48.99 |
| ATOM | 7552 | CB | TYR | 4154 | 48.683 | −11.264 | 95.505 | 1.00 | 48.01 |
| ATOM | 7553 | CG | TYR | 4154 | 48.548 | −10.144 | 96.498 | 1.00 | 47.32 |
| ATOM | 7554 | CD1 | TYR | 4154 | 49.667 | −9.629 | 97.152 | 1.00 | 46.78 |
| ATOM | 7555 | CE1 | TYR | 4154 | 49.560 | −8.552 | 98.023 | 1.00 | 47.07 |
| ATOM | 7556 | CD2 | TYR | 4154 | 47.309 | −9.555 | 96.740 | 1.00 | 47.24 |
| ATOM | 7557 | CE2 | TYR | 4154 | 47.186 | −8.474 | 97.605 | 1.00 | 47.94 |
| ATOM | 7558 | CZ | TYR | 4154 | 48.317 | −7.977 | 98.246 | 1.00 | 47.99 |
| ATOM | 7559 | OH | TYR | 4154 | 48.198 | −6.906 | 99.099 | 1.00 | 48.10 |
| ATOM | 7560 | C | TYR | 4154 | 48.211 | −9.770 | 93.560 | 1.00 | 48.32 |
| ATOM | 7561 | O | TYR | 4154 | 48.407 | −8.560 | 93.693 | 1.00 | 48.04 |
| ATOM | 7562 | N | TRP | 4155 | 47.164 | −10.264 | 92.906 | 1.00 | 47.77 |
| ATOM | 7563 | CA | TRP | 4155 | 46.162 | −9.387 | 92.305 | 1.00 | 47.16 |
| ATOM | 7564 | CB | TRP | 4155 | 45.243 | −10.158 | 91.349 | 1.00 | 44.47 |
| ATOM | 7565 | CG | TRP | 4155 | 45.964 | −11.008 | 90.361 | 1.00 | 41.47 |
| ATOM | 7566 | CD2 | TRP | 4155 | 46.850 | −10.561 | 89.331 | 1.00 | 40.49 |
| ATOM | 7567 | CE2 | TRP | 4155 | 47.321 | −11.710 | 88.664 | 1.00 | 39.93 |
| ATOM | 7568 | CE3 | TRP | 4155 | 47.294 | −9.299 | 88.906 | 1.00 | 40.11 |
| ATOM | 7569 | CD1 | TRP | 4155 | 45.931 | −12.363 | 90.278 | 1.00 | 40.57 |
| ATOM | 7570 | NE1 | TRP | 4155 | 46.743 | −12.795 | 89.263 | 1.00 | 39.94 |
| ATOM | 7571 | CZ2 | TRP | 4155 | 48.218 | −11.639 | 87.594 | 1.00 | 39.83 |
| ATOM | 7572 | CZ3 | TRP | 4155 | 48.182 | −9.227 | 87.845 | 1.00 | 39.17 |
| ATOM | 7573 | CH2 | TRP | 4155 | 48.637 | −10.393 | 87.201 | 1.00 | 39.98 |
| ATOM | 7574 | C | TRP | 4155 | 45.317 | −8.784 | 93.401 | 1.00 | 47.62 |
| ATOM | 7575 | O | TRP | 4155 | 44.916 | −9.477 | 94.328 | 1.00 | 47.64 |
| ATOM | 7576 | N | THR | 4156 | 45.042 | −7.493 | 93.291 | 1.00 | 48.96 |
| ATOM | 7577 | CA | THR | 4156 | 44.236 | −6.813 | 94.287 | 1.00 | 50.26 |
| ATOM | 7578 | CB | THR | 4156 | 44.804 | −5.421 | 94.576 | 1.00 | 50.10 |
| ATOM | 7579 | OG1 | TER | 4156 | 45.047 | −4.737 | 93.343 | 1.00 | 51.09 |
| ATOM | 7580 | CG2 | THR | 4156 | 46.113 | −5.540 | 95.328 | 1.00 | 50.10 |
| ATOM | 7581 | C | THR | 4156 | 42.781 | −6.712 | 93.843 | 1.00 | 51.28 |
| ATOM | 7582 | O | THR | 4156 | 41.890 | −6.563 | 94.669 | 1.00 | 51.53 |
| ATOM | 7583 | N | SER | 4157 | 42.544 | −6.803 | 92.537 | 1.00 | 52.51 |
| ATOM | 7584 | CA | SER | 4157 | 41.186 | −6.742 | 91.996 | 1.00 | 53.88 |
| ATOM | 7585 | CB | SER | 4157 | 40.887 | −5.355 | 91.407 | 1.00 | 53.95 |
| ATOM | 7586 | OG | SER | 4157 | 40.989 | −4.345 | 92.394 | 1.00 | 54.88 |
| ATOM | 7587 | C | SER | 4157 | 40.983 | −7.797 | 90.914 | 1.00 | 54.71 |
| ATOM | 7588 | O | SER | 4157 | 40.637 | −7.471 | 89.776 | 1.00 | 54.69 |
| ATOM | 7589 | N | PRO | 4158 | 41.184 | −9.082 | 91.256 | 1.00 | 55.82 |
| ATOM | 7590 | CD | PRO | 4158 | 41.337 | −9.642 | 92.608 | 1.00 | 55.82 |
| ATOM | 7591 | CA | PRO | 4158 | 41.011 | −10.164 | 90.276 | 1.00 | 56.78 |
| ATOM | 7592 | CB | PRO | 4158 | 41.181 | −11.428 | 91.120 | 1.00 | 56.02 |
| ATOM | 7593 | CG | PRO | 4158 | 40.682 | −11.000 | 92.453 | 1.00 | 55.91 |
| ATOM | 7594 | C | PRO | 4158 | 39.646 | −10.097 | 89.606 | 1.00 | 57.50 |
| ATOM | 7595 | O | PRO | 4158 | 39.453 | −10.588 | 88.495 | 1.00 | 57.14 |
| ATOM | 7596 | N | GLU | 4159 | 38.708 | −9.478 | 90.308 | 1.00 | 58.50 |
| ATOM | 7597 | CA | GLU | 4159 | 37.351 | −9.301 | 89.834 | 1.00 | 59.46 |
| ATOM | 7598 | CB | GLU | 4159 | 36.582 | −8.561 | 90.924 | 1.00 | 60.97 |
| ATOM | 7599 | CG | GLU | 4159 | 35.135 | −8.273 | 90.648 | 1.00 | 63.43 |
| ATOM | 7600 | CD | GLU | 4159 | 34.353 | −8.045 | 91.942 | 1.00 | 65.30 |
| ATOM | 7601 | OE1 | GLU | 4159 | 33.943 | −9.050 | 92.579 | 1.00 | 65.46 |
| ATOM | 7602 | OE2 | GLU | 4159 | 34.166 | −6.863 | 92.331 | 1.00 | 66.01 |
| ATOM | 7603 | C | GLU | 4159 | 37.338 | −8.526 | 88.513 | 1.00 | 59.05 |
| ATOM | 7604 | O | GLU | 4159 | 36.648 | −8.917 | 87.565 | 1.00 | 59.69 |
| ATOM | 7605 | N | LYS | 4160 | 38.101 | −7.437 | 88.433 | 1.00 | 58.13 |
| ATOM | 7606 | CA | LYS | 4160 | 38.113 | −6.676 | 87.191 | 1.00 | 57.42 |
| ATOM | 7607 | CB | LYS | 4160 | 38.253 | −5.165 | 87.439 | 1.00 | 57.32 |
| ATOM | 7608 | CG | LYS | 4160 | 39.152 | −4.710 | 88.564 | 1.00 | 57.93 |
| ATOM | 7609 | CD | LYS | 4160 | 39.249 | −3.173 | 88.551 | 1.00 | 58.19 |
| ATOM | 7610 | CE | LYS | 4160 | 37.881 | −2.492 | 88.726 | 1.00 | 57.90 |
| ATOM | 7611 | NZ | LYS | 4160 | 37.872 | −1.045 | 88.304 | 1.00 | 57.60 |
| ATOM | 7612 | C | LYS | 4160 | 39.112 | −7.162 | 86.142 | 1.00 | 56.83 |
| ATOM | 7613 | O | LYS | 4160 | 39.502 | −6.420 | 85.239 | 1.00 | 56.76 |
| ATOM | 7614 | N | MSE | 4161 | 39.498 | −8.429 | 86.254 | 1.00 | 55.86 |
| ATOM | 7615 | CA | MSE | 4161 | 40.416 | −9.049 | 85.300 | 1.00 | 55.21 |
| ATOM | 7616 | CB | MSE | 4161 | 41.659 | −9.587 | 86.028 | 1.00 | 53.87 |
| ATOM | 7617 | CG | MSE | 4161 | 42.635 | −8.505 | 86.512 | 1.00 | 52.07 |
| ATOM | 7618 | SE | MSE | 4161 | 43.979 | −9.156 | 87.522 | 1.00 | 48.65 |
| ATOM | 7619 | CE | MSE | 4161 | 44.891 | −10.063 | 86.318 | 1.00 | 49.23 |
| ATOM | 7620 | C | MSE | 4161 | 39.671 | −10.198 | 84.613 | 1.00 | 55.38 |
| ATOM | 7621 | O | MSE | 4161 | 40.269 | −11.039 | 83.938 | 1.00 | 55.27 |
| ATOM | 7622 | N | GLU | 4162 | 38.352 | −10.213 | 84.796 | 1.00 | 55.47 |
| ATOM | 7623 | CA | GLU | 4162 | 37.484 | −11.246 | 84.239 | 1.00 | 54.95 |
| ATOM | 7624 | CE | GLU | 4162 | 36.044 | −11.023 | 84.716 | 1.00 | 56.55 |
| ATOM | 7625 | CG | GLU | 4162 | 35.437 | −12.240 | 85.388 | 1.00 | 58.02 |
| ATOM | 7626 | CD | GLU | 4162 | 36.185 | −12.631 | 86.651 | 1.00 | 59.59 |
| ATOM | 7627 | OE1 | GLU | 4162 | 37.440 | −12.695 | 86.603 | 1.00 | 59.62 |
| ATOM | 7628 | OE2 | GLU | 4162 | 35.516 | −12.880 | 87.685 | 1.00 | 59.60 |
| ATOM | 7629 | C | GLU | 4162 | 37.505 | −11.305 | 82.718 | 1.00 | 53.61 |
| ATOM | 7630 | O | GLU | 4162 | 37.811 | −12.342 | 82.133 | 1.00 | 53.64 |
| ATOM | 7631 | N | LYS | 4163 | 37.167 | −10.182 | 82.095 | 1.00 | 51.83 |
| ATOM | 7632 | CA | LYS | 4163 | 37.118 | −10.034 | 80.644 | 1.00 | 50.06 |
| ATOM | 7633 | CB | LYS | 4163 | 36.588 | −8.637 | 80.330 | 1.00 | 49.55 |
| ATOM | 7634 | CG | LYS | 4163 | 36.521 | −8.249 | 78.873 | 1.00 | 49.30 |
| ATOM | 7635 | CD | LYS | 4163 | 36.003 | −6.821 | 78.775 | 1.00 | 48.47 |
| ATOM | 7636 | CE | LYS | 4163 | 36.058 | −6.293 | 77.363 | 1.00 | 48.43 |
| ATOM | 7637 | NZ | LYS | 4163 | 35.706 | −4.849 | 77.302 | 1.00 | 48.04 |
| ATOM | 7638 | C | LYS | 4163 | 38.480 | −10.256 | 79.976 | 1.00 | 48.97 |
| ATOM | 7639 | O | LYS | 4163 | 39.337 | −9.372 | 79.987 | 1.00 | 48.98 |
| ATOM | 7640 | N | LYS | 4164 | 38.666 | −11.436 | 79.388 | 1.00 | 47.35 |
| ATOM | 7641 | CA | LYS | 4164 | 39.922 | −11.782 | 78.727 | 1.00 | 45.87 |
| ATOM | 7642 | CB | LYS | 4164 | 40.114 | −13.305 | 78.738 | 1.00 | 46.39 |
| ATOM | 7643 | CG | LYS | 4164 | 41.564 | −13.752 | 78.601 | 1.00 | 47.97 |
| ATOM | 7644 | CD | LYS | 4164 | 41.698 | −15.268 | 78.666 | 1.00 | 49.30 |
| ATOM | 7645 | CE | LYS | 4164 | 43.095 | −15.683 | 79.123 | 1.00 | 50.07 |
| ATOM | 7646 | NZ | LYS | 4164 | 43.345 | −15.286 | 80.550 | 1.00 | 51.37 |
| ATOM | 7647 | C | LYS | 4164 | 39.999 | −11.267 | 77.293 | 1.00 | 43.96 |
| ATOM | 7648 | O | LYS | 4164 | 41.036 | −10.766 | 76.864 | 1.00 | 43.43 |
| ATOM | 7649 | N | LEU | 4165 | 38.902 | −11.402 | 76.552 | 1.00 | 41.90 |
| ATOM | 7650 | CA | LEU | 4165 | 38.868 | −10.940 | 75.176 | 1.00 | 40.13 |
| ATOM | 7651 | CB | LEU | 4165 | 37.992 | −11.857 | 74.329 | 1.00 | 39.40 |
| ATOM | 7652 | CG | LEU | 4165 | 37.708 | −11.291 | 72.936 | 1.00 | 39.22 |
| ATOM | 7653 | CD1 | LEU | 4165 | 39.020 | −11.054 | 72.194 | 1.00 | 38.71 |
| ATOM | 7654 | CD2 | LEU | 4165 | 36.823 | −12.243 | 72.173 | 1.00 | 39.03 |
| ATOM | 7655 | C | LEU | 4165 | 38.343 | −9.517 | 75.066 | 1.00 | 39.07 |
| ATOM | 7656 | O | LEU | 4165 | 37.243 | −9.204 | 75.516 | 1.00 | 38.91 |
| ATOM | 7657 | N | HIS | 4166 | 39.145 | −8.655 | 74.464 | 1.00 | 37.79 |
| ATOM | 7658 | CA | HIS | 4166 | 38.762 | −7.269 | 74.253 | 1.00 | 36.51 |
| ATOM | 7659 | CB | HIS | 4166 | 39.864 | −6.332 | 74.748 | 1.00 | 37.40 |
| ATOM | 7660 | CG | HIS | 4166 | 39.927 | −6.203 | 76.235 | 1.00 | 38.51 |
| ATOM | 7661 | CD2 | HIS | 4166 | 40.353 | −7.064 | 77.190 | 1.00 | 39.41 |
| ATOM | 7662 | ND1 | HIS | 4166 | 39.497 | −5.074 | 76.901 | 1.00 | 39.08 |
| ATOM | 7663 | CE1 | HIS | 4166 | 39.655 | −5.246 | 78.202 | 1.00 | 39.42 |
| ATOM | 7664 | NE2 | HIS | 4166 | 40.172 | −6.446 | 78.405 | 1.00 | 39.60 |
| ATOM | 7665 | C | HIS | 4166 | 38.580 | −7.088 | 72.750 | 1.00 | 34.98 |
| ATOM | 7666 | O | HIS | 4166 | 39.550 | −7.119 | 71.991 | 1.00 | 34.40 |
| ATOM | 7667 | N | ALA | 4167 | 37.339 | −6.929 | 72.316 | 1.00 | 33.25 |
| ATOM | 7668 | CA | ALA | 4167 | 37.073 | −6.736 | 70.903 | 1.00 | 32.03 |
| ATOM | 7669 | CB | ALA | 4167 | 36.045 | −7.750 | 70.425 | 1.00 | 31.56 |
| ATOM | 7670 | C | ALA | 4167 | 36.545 | −5.318 | 70.738 | 1.00 | 30.93 |
| ATOM | 7671 | O | ALA | 4167 | 35.623 | −4.904 | 71.447 | 1.00 | 31.19 |
| ATOM | 7672 | N | VAL | 4168 | 37.134 | −4.561 | 69.821 | 1.00 | 29.63 |
| ATOM | 7673 | CA | VAL | 4168 | 36.687 | −3.191 | 69.600 | 1.00 | 28.41 |
| ATOM | 7674 | CB | VAL | 4168 | 37.477 | −2.161 | 70.461 | 1.00 | 28.76 |
| ATOM | 7675 | CG1 | VAL | 4168 | 37.347 | −2.484 | 71.932 | 1.00 | 28.09 |
| ATOM | 7676 | CG2 | VAL | 4168 | 38.941 | −2.151 | 70.053 | 1.00 | 28.20 |
| ATOM | 7677 | C | VAL | 4168 | 36.850 | −2.786 | 68.152 | 1.00 | 27.74 |
| ATOM | 7678 | O | VAL | 4168 | 37.631 | −3.382 | 67.409 | 1.00 | 28.42 |
| ATOM | 7679 | N | PRO | 4169 | 36.095 | −1.767 | 67.727 | 1.00 | 27.18 |
| ATOM | 7680 | CD | PRO | 4169 | 35.127 | −0.968 | 68.507 | 1.00 | 27.29 |
| ATOM | 7681 | CA | PRO | 4169 | 36.182 | −1.292 | 66.350 | 1.00 | 26.53 |
| ATOM | 7682 | CB | PRO | 4169 | 34.968 | −0.376 | 66.243 | 1.00 | 27.32 |
| ATOM | 7683 | CG | PRO | 4169 | 34.878 | 0.220 | 67.614 | 1.00 | 25.97 |
| ATOM | 7684 | C | PRO | 4169 | 37.500 | −0.536 | 66.273 | 1.00 | 26.41 |
| ATOM | 7685 | O | PRO | 4169 | 38.039 | −0.136 | 67.307 | 1.00 | 26.35 |
| ATOM | 7686 | N | ALA | 4170 | 38.019 | −0.343 | 65.068 | 1.00 | 26.07 |
| ATOM | 7687 | CA | ALA | 4170 | 39.274 | 0.364 | 64.885 | 1.00 | 26.05 |
| ATOM | 7688 | CB | ALA | 4170 | 39.673 | 0.311 | 63.412 | 1.00 | 24.81 |
| ATOM | 7689 | C | ALA | 4170 | 39.141 | 1.811 | 65.360 | 1.00 | 26.45 |
| ATOM | 7690 | O | ALA | 4170 | 38.041 | 2.376 | 65.342 | 1.00 | 27.06 |
| ATOM | 7691 | N | ALA | 4171 | 40.259 | 2.383 | 65.813 | 1.00 | 26.45 |
| ATOM | 7692 | CA | ALA | 4171 | 40.333 | 3.771 | 66.280 | 1.00 | 26.51 |
| ATOM | 7693 | CB | ALA | 4171 | 39.442 | 4.684 | 65.412 | 1.00 | 24.70 |
| ATOM | 7694 | C | ALA | 4171 | 39.990 | 3.968 | 67.742 | 1.00 | 27.09 |
| ATOM | 7695 | O | ALA | 4171 | 40.227 | 5.039 | 68.291 | 1.00 | 28.09 |
| ATOM | 7696 | N | LYS | 4172 | 39.427 | 2.948 | 68.373 | 1.00 | 27.28 |
| ATOM | 7697 | CA | LYS | 4172 | 39.058 | 3.053 | 69.777 | 1.00 | 27.28 |
| ATOM | 7698 | CB | LYS | 4172 | 38.132 | 1.896 | 70.139 | 1.00 | 28.17 |
| ATOM | 7699 | CG | LYS | 4172 | 37.600 | 1.947 | 71.549 | 1.00 | 28.70 |
| ATOM | 7700 | CD | LYS | 4172 | 36.129 | 2.301 | 71.575 | 1.00 | 28.68 |
| ATOM | 7701 | CE | LYS | 4172 | 35.698 | 2.549 | 72.995 | 1.00 | 29.82 |
| ATOM | 7702 | NZ | LYS | 4172 | 36.361 | 1.602 | 73.952 | 1.00 | 30.11 |
| ATOM | 7703 | C | LYS | 4172 | 40.290 | 3.011 | 70.677 | 1.00 | 27.26 |
| ATOM | 7704 | O | LYS | 4172 | 41.341 | 2.495 | 70.291 | 1.00 | 26.80 |
| ATOM | 7705 | N | THR | 4173 | 40.156 | 3.560 | 71.878 | 1.00 | 27.62 |
| ATOM | 7706 | CA | THR | 4173 | 41.230 | 3.549 | 72.862 | 1.00 | 27.84 |
| ATOM | 7707 | CB | THR | 4173 | 41.134 | 4.770 | 73.803 | 1.00 | 27.74 |
| ATOM | 7708 | OG1 | THR | 4173 | 41.514 | 5.950 | 73.082 | 1.00 | 27.96 |
| ATOM | 7709 | CG2 | THR | 4173 | 42.035 | 4.590 | 75.026 | 1.00 | 27.01 |
| ATOM | 7710 | C | THR | 4173 | 41.066 | 2.273 | 73.690 | 1.00 | 28.59 |
| ATOM | 7711 | O | THR | 4173 | 39.962 | 1.961 | 74.130 | 1.00 | 29.65 |
| ATOM | 7712 | N | VAL | 4174 | 42.154 | 1.537 | 73.895 | 1.00 | 28.62 |
| ATOM | 7713 | CA | VAL | 4174 | 42.101 | 0.301 | 74.660 | 1.00 | 28.78 |
| ATOM | 7714 | CB | VAL | 4174 | 42.589 | −0.896 | 73.820 | 1.00 | 28.82 |
| ATOM | 7715 | CG1 | VAL | 4174 | 42.842 | −2.100 | 74.710 | 1.00 | 27.13 |
| ATOM | 7716 | CG2 | VAL | 4174 | 41.555 | −1.235 | 72.765 | 1.00 | 28.27 |
| ATOM | 7717 | C | VAL | 4174 | 42.969 | 0.408 | 75.898 | 1.00 | 30.05 |
| ATOM | 7718 | O | VAL | 4174 | 44.092 | 0.919 | 75.848 | 1.00 | 30.16 |
| ATOM | 7719 | N | LYS | 4175 | 42.448 | −0.089 | 77.012 | 1.00 | 30.82 |
| ATOM | 7720 | CA | LYS | 4175 | 43.183 | −0.040 | 78.258 | 1.00 | 32.13 |
| ATOM | 7721 | CB | LYS | 4175 | 42.595 | 1.049 | 79.148 | 1.00 | 33.15 |
| ATOM | 7722 | CG | LYS | 4175 | 43.210 | 1.161 | 80.537 | 1.00 | 35.09 |
| ATOM | 7723 | CD | LYS | 4175 | 42.460 | 2.223 | 81.302 | 1.00 | 36.73 |
| ATOM | 7724 | CE | LYS | 4175 | 43.045 | 2.541 | 82.646 | 1.00 | 37.74 |
| ATOM | 7725 | NZ | LYS | 4175 | 42.157 | 3.557 | 83.280 | 1.00 | 38.46 |
| ATOM | 7726 | C | LYS | 4175 | 43.160 | −1.376 | 78.985 | 1.00 | 32.37 |
| ATOM | 7727 | O | LYS | 4175 | 42.098 | −1.908 | 79.303 | 1.00 | 32.60 |
| ATOM | 7728 | N | PHE | 4176 | 44.344 | −1.920 | 79.236 | 1.00 | 33.48 |
| ATOM | 7729 | CA | PHE | 4176 | 44.481 | −3.186 | 79.956 | 1.00 | 33.86 |
| ATOM | 7730 | CB | PHE | 4176 | 45.414 | −4.143 | 79.217 | 1.00 | 31.28 |
| ATOM | 7731 | CG | PHE | 4176 | 44.939 | −4.543 | 77.856 | 1.00 | 30.29 |
| ATOM | 7732 | CD1 | PHE | 4176 | 43.671 | −5.081 | 77.673 | 1.00 | 30.11 |
| ATOM | 7733 | CD2 | PHE | 4176 | 45.789 | −4.432 | 76.752 | 1.00 | 29.92 |
| ATOM | 7734 | CE1 | PHE | 4176 | 43.259 | −5.508 | 76.409 | 1.00 | 29.19 |
| ATOM | 7735 | CE2 | PHE | 4176 | 45.390 | −4.854 | 75.489 | 1.00 | 28.32 |
| ATOM | 7736 | CZ | PHE | 4176 | 44.120 | −5.394 | 75.319 | 1.00 | 28.76 |
| ATOM | 7737 | C | PHE | 4176 | 45.080 | −2.893 | 81.332 | 1.00 | 35.21 |
| ATOM | 7738 | O | PHE | 4176 | 45.987 | −2.067 | 81.465 | 1.00 | 35.41 |
| ATOM | 7739 | N | LYS | 4177 | 44.588 | −3.573 | 82.357 | 1.00 | 36.65 |
| ATOM | 7740 | CA | LYS | 4177 | 45.112 | −3.354 | 83.693 | 1.00 | 38.29 |
| ATOM | 7741 | CB | LYS | 4177 | 44.213 | −2.379 | 84.443 | 1.00 | 39.54 |
| ATOM | 7742 | CG | LYS | 4177 | 42.770 | −2.806 | 84.466 | 1.00 | 42.71 |
| ATOM | 7743 | CD | LYS | 4177 | 41.870 | −1.706 | 84.994 | 1.00 | 45.11 |
| ATOM | 7744 | CE | LYS | 4177 | 40.397 | −2.123 | 84.907 | 1.00 | 47.24 |
| ATOM | 7745 | NZ | LYS | 4177 | 39.462 | −1.101 | 85.494 | 1.00 | 49.21 |
| ATOM | 7746 | C | LYS | 4177 | 45.255 | −4.635 | 84.494 | 1.00 | 38.33 |
| ATOM | 7747 | O | LYS | 4177 | 44.504 | −5.585 | 84.316 | 1.00 | 37.60 |
| ATOM | 7748 | N | CYS | 4178 | 46.243 | −4.646 | 85.377 | 1.00 | 39.49 |
| ATOM | 7749 | CA | CYS | 4178 | 46.489 | −5.789 | 86.235 | 1.00 | 40.34 |
| ATOM | 7750 | CB | CYS | 4178 | 47.610 | −6.617 | 85.650 | 1.00 | 40.76 |
| ATOM | 7751 | SG | CYS | 4178 | 47.096 | −7.222 | 84.043 | 1.00 | 42.54 |
| ATOM | 7752 | C | CYS | 4178 | 46.824 | −5.286 | 87.618 | 1.00 | 40.40 |
| ATOM | 7753 | O | CYS | 4178 | 47.962 | −5.377 | 88.071 | 1.00 | 40.59 |
| ATOM | 7754 | N | PRO | 4179 | 45.815 | −4.734 | 88.309 | 1.00 | 40.92 |
| ATOM | 7755 | CD | PRO | 4179 | 44.394 | −4.747 | 87.919 | 1.00 | 40.19 |
| ATOM | 7756 | CA | PRO | 4179 | 45.982 | −4.195 | 89.666 | 1.00 | 41.60 |
| ATOM | 7757 | CB | PRO | 4179 | 44.557 | −3.793 | 90.073 | 1.00 | 41.00 |
| ATOM | 7758 | CG | PRO | 4179 | 43.829 | −3.641 | 88.771 | 1.00 | 40.65 |
| ATOM | 7759 | C | PRO | 4179 | 46.548 | −5.263 | 90.584 | 1.00 | 42.42 |
| ATOM | 7760 | O | PRO | 4179 | 45.959 | −6.338 | 90.743 | 1.00 | 42.57 |
| ATOM | 7761 | N | SER | 4180 | 47.696 | −4.971 | 91.177 | 1.00 | 43.48 |
| ATOM | 7762 | CA | SER | 4180 | 48.329 | −5.921 | 92.071 | 1.00 | 44.76 |
| ATOM | 7763 | CB | SER | 4180 | 49.270 | −6.825 | 91.282 | 1.00 | 44.24 |
| ATOM | 7764 | OG | SER | 4180 | 50.187 | −6.052 | 90.544 | 1.00 | 44.37 |
| ATOM | 7765 | C | SER | 4180 | 49.088 | −5.204 | 93.169 | 1.00 | 45.70 |
| ATOM | 7766 | O | SER | 4180 | 49.080 | −3.970 | 93.250 | 1.00 | 45.95 |
| ATOM | 7767 | N | SER | 4181 | 49.739 | −5.983 | 94.022 | 1.00 | 46.53 |
| ATOM | 7768 | CA | SER | 4181 | 50.489 | −5.413 | 95.124 | 1.00 | 47.51 |
| ATOM | 7769 | CB | SER | 4181 | 49.523 | −5.031 | 96.250 | 1.00 | 47.68 |
| ATOM | 7770 | OG | SER | 4181 | 50.087 | −4.049 | 97.106 | 1.00 | 48.24 |
| ATOM | 7771 | C | SER | 4181 | 51.519 | −6.408 | 95.631 | 1.00 | 47.76 |
| ATOM | 7772 | O | SER | 4181 | 51.648 | −7.508 | 95.098 | 1.00 | 48.14 |
| ATOM | 7773 | N | GLY | 4182 | 52.246 | −6.010 | 96.668 | 1.00 | 47.93 |
| ATOM | 7774 | CA | GLY | 4182 | 53.258 | −6.869 | 97.243 | 1.00 | 48.71 |
| ATOM | 7775 | C | GLY | 4182 | 54.398 | −6.016 | 97.748 | 1.00 | 49.17 |
| ATOM | 7776 | O | GLY | 4182 | 54.499 | −4.837 | 97.397 | 1.00 | 49.15 |
| ATOM | 7777 | N | THR | 4183 | 55.253 | −6.589 | 98.586 | 1.00 | 49.38 |
| ATOM | 7778 | CA | THR | 4183 | 56.378 | −5.828 | 99.100 | 1.00 | 49.22 |
| ATOM | 7779 | CB | THR | 4183 | 56.116 | −5.285 | 100.526 | 1.00 | 49.53 |
| ATOM | 7780 | OG1 | THR | 4183 | 56.678 | −6.181 | 101.492 | 1.00 | 51.52 |
| ATOM | 7781 | CG2 | THR | 4183 | 54.609 | −5.128 | 100.778 | 1.00 | 48.85 |
| ATOM | 7782 | C | THR | 4183 | 57.655 | −6.654 | 99.082 | 1.00 | 48.59 |
| ATOM | 7783 | O | THR | 4183 | 57.678 | −7.807 | 99.507 | 1.00 | 48.21 |
| ATOM | 7784 | N | PRO | 4184 | 58.736 | −6.068 | 98.556 | 1.00 | 48.61 |
| ATOM | 7785 | CD | PRO | 4184 | 60.016 | −6.735 | 98.261 | 1.00 | 48.28 |
| ATOM | 7786 | CA | PRO | 4184 | 58.719 | −4.702 | 98.022 | 1.00 | 48.79 |
| ATOM | 7787 | CB | PRO | 4184 | 60.176 | −4.470 | 97.636 | 1.00 | 48.81 |
| ATOM | 7788 | CG | PRO | 4184 | 60.612 | −5.836 | 97.199 | 1.00 | 49.01 |
| ATOM | 7789 | C | PRO | 4184 | 57.764 | −4.560 | 96.833 | 1.00 | 48.80 |
| ATOM | 7790 | O | PRO | 4184 | 57.331 | −5.560 | 96.252 | 1.00 | 48.76 |
| ATOM | 7791 | N | GLN | 4185 | 57.430 | −3.317 | 96.491 | 1.00 | 48.74 |
| ATOM | 7792 | CA | GLN | 4185 | 56.530 | −3.040 | 95.376 | 1.00 | 48.71 |
| ATOM | 7793 | CB | GLN | 4185 | 56.439 | −1.528 | 95.122 | 1.00 | 49.24 |
| ATOM | 7794 | CG | GLN | 4185 | 55.136 | −1.097 | 94.455 | 1.00 | 50.72 |
| ATOM | 7795 | CD | GLN | 4185 | 53.924 | −1.312 | 95.360 | 1.00 | 51.51 |
| ATOM | 7796 | OE1 | GLN | 4185 | 52.894 | −1.849 | 94.937 | 1.00 | 51.11 |
| ATOM | 7797 | NE2 | GLN | 4185 | 54.047 | −0.887 | 96.616 | 1.00 | 52.16 |
| ATOM | 7798 | C | GLN | 4185 | 57.042 | −3.739 | 94.115 | 1.00 | 48.26 |
| ATOM | 7799 | O | GLN | 4185 | 58.192 | −3.545 | 93.706 | 1.00 | 48.16 |
| ATOM | 7800 | N | PRO | 4186 | 56.193 | −4.567 | 93.483 | 1.00 | 47.49 |
| ATOM | 7801 | CD | PRO | 4186 | 54.851 | −4.975 | 93.937 | 1.00 | 47.00 |
| ATOM | 7802 | CA | PRO | 4186 | 56.580 | −5.292 | 92.266 | 1.00 | 46.92 |
| ATOM | 7803 | CB | PRO | 4186 | 55.516 | −6.374 | 92.165 | 1.00 | 46.95 |
| ATOM | 7804 | CG | PRO | 4186 | 54.306 | −5.693 | 92.728 | 1.00 | 47.15 |
| ATOM | 7805 | C | PRO | 4186 | 56.623 | −4.414 | 91.021 | 1.00 | 46.19 |
| ATOM | 7806 | O | PRO | 4186 | 55.906 | −3.422 | 90.935 | 1.00 | 46.51 |
| ATOM | 7807 | N | THR | 4187 | 57.474 | −4.778 | 90.067 | 1.00 | 45.33 |
| ATOM | 7808 | CA | THR | 4187 | 57.583 | −4.033 | 88.818 | 1.00 | 44.44 |
| ATOM | 7809 | CB | THR | 4187 | 58.964 | −4.211 | 88.157 | 1.00 | 44.82 |
| ATOM | 7810 | OG1 | THR | 4187 | 59.232 | −5.609 | 87.966 | 1.00 | 45.14 |
| ATOM | 7811 | CG2 | THR | 4187 | 60.053 | −3.580 | 89.019 | 1.00 | 45.18 |
| ATOM | 7812 | C | THR | 4187 | 56.528 | −4.538 | 87.848 | 1.00 | 43.71 |
| ATOM | 7813 | O | THR | 4187 | 55.965 | −5.618 | 88.031 | 1.00 | 43.78 |
| ATOM | 7814 | N | LEU | 4188 | 56.273 | −3.759 | 86.806 | 1.00 | 42.68 |
| ATOM | 7815 | CA | LEU | 4188 | 55.274 | −4.122 | 85.815 | 1.00 | 41.11 |
| ATOM | 7816 | CB | LEU | 4188 | 54.005 | −3.294 | 86.046 | 1.00 | 41.25 |
| ATOM | 7817 | CG | LEU | 4188 | 52.820 | −3.363 | 85.077 | 1.00 | 41.06 |
| ATOM | 7818 | CD1 | LEU | 4188 | 51.704 | −2.457 | 85.562 | 1.00 | 41.31 |
| ATOM | 7819 | CD2 | LEU | 4188 | 53.246 | −2.912 | 83.708 | 1.00 | 42.35 |
| ATOM | 7820 | C | LEU | 4188 | 55.775 | −3.894 | 84.401 | 1.00 | 40.23 |
| ATOM | 7821 | O | LEU | 4188 | 56.121 | −2.774 | 84.035 | 1.00 | 40.56 |
| ATOM | 7822 | N | ARG | 4189 | 55.821 | −4.954 | 83.606 | 1.00 | 39.20 |
| ATOM | 7823 | CA | ARG | 4189 | 56.228 | −4.820 | 82.219 | 1.00 | 38.28 |
| ATOM | 7824 | CB | ARG | 4189 | 57.659 | −5.328 | 82.020 | 1.00 | 39.11 |
| ATOM | 7825 | CG | ARG | 4189 | 57.859 | −6.822 | 82.128 | 1.00 | 41.52 |
| ATOM | 7826 | CD | ARG | 4189 | 59.342 | −7.166 | 82.418 | 1.00 | 42.44 |
| ATOM | 7827 | NE | ARG | 4189 | 59.610 | −8.589 | 82.214 | 1.00 | 43.99 |
| ATOM | 7828 | CZ | ARG | 4189 | 59.717 | −9.149 | 81.011 | 1.00 | 45.40 |
| ATOM | 7829 | NH1 | ARG | 4189 | 59.592 | −8.398 | 79.920 | 1.00 | 45.98 |
| ATOM | 7830 | NH2 | ARG | 4189 | 59.907 | −10.458 | 80.886 | 1.00 | 45.64 |
| ATOM | 7831 | C | ARG | 4189 | 55.215 | −5.566 | 81.342 | 1.00 | 37.22 |
| ATOM | 7832 | O | ARG | 4189 | 54.583 | −6.531 | 81.787 | 1.00 | 37.17 |
| ATOM | 7833 | N | TRP | 4190 | 55.027 | −5.096 | 80.113 | 1.00 | 35.15 |
| ATOM | 7834 | CA | TRP | 4190 | 54.067 | −5.719 | 79.216 | 1.00 | 33.29 |
| ATOM | 7835 | CB | TRP | 4190 | 53.036 | −4.698 | 78.720 | 1.00 | 31.42 |
| ATOM | 7836 | CG | TRP | 4190 | 52.126 | −4.174 | 79.771 | 1.00 | 29.82 |
| ATOM | 7837 | CD2 | TRP | 4190 | 50.824 | −4.670 | 80.096 | 1.00 | 29.46 |
| ATOM | 7838 | CE2 | TRP | 4190 | 50.322 | −3.870 | 81.149 | 1.00 | 28.75 |
| ATOM | 7839 | CE3 | TRP | 4190 | 50.028 | −5.713 | 79.598 | 1.00 | 29.10 |
| ATOM | 7840 | CD1 | TRP | 4190 | 52.359 | −3.128 | 80.614 | 1.00 | 28.76 |
| ATOM | 7841 | NE1 | TRP | 4190 | 51.278 | −2.937 | 81.446 | 1.00 | 28.14 |
| ATOM | 7842 | CZ2 | TRP | 4190 | 49.052 | −4.080 | 81.714 | 1.00 | 28.50 |
| ATOM | 7843 | CZ3 | TRP | 4190 | 48.763 | −5.921 | 80.163 | 1.00 | 28.12 |
| ATOM | 7844 | CH2 | TRP | 4190 | 48.293 | −5.107 | 81.208 | 1.00 | 27.95 |
| ATOM | 7845 | C | TRP | 4190 | 54.708 | −6.371 | 78.014 | 1.00 | 33.14 |
| ATOM | 7846 | O | TRP | 4190 | 55.738 | −5.914 | 77.525 | 1.00 | 33.83 |
| ATOM | 7847 | N | LEU | 4191 | 54.086 | −7.441 | 77.540 | 1.00 | 32.82 |
| ATOM | 7848 | CA | LEU | 4191 | 54.570 | −8.148 | 76.365 | 1.00 | 33.52 |
| ATOM | 7849 | CB | LEU | 4191 | 54.933 | −9.600 | 76.697 | 1.00 | 33.71 |
| ATOM | 7850 | CG | LEU | 4191 | 56.032 | −10.019 | 77.688 | 1.00 | 34.46 |
| ATOM | 7851 | CD1 | LEU | 4191 | 56.150 | −11.544 | 77.615 | 1.00 | 33.22 |
| ATOM | 7852 | CD2 | LEU | 4191 | 57.385 | −9.391 | 77.349 | 1.00 | 32.66 |
| ATOM | 7853 | C | LEU | 4191 | 53.466 | −8.174 | 75.306 | 1.00 | 33.56 |
| ATOM | 7854 | O | LEU | 4191 | 52.279 | −8.128 | 75.626 | 1.00 | 33.00 |
| ATOM | 7855 | N | LYS | 4192 | 53.868 | −8.229 | 74.042 | 1.00 | 33.71 |
| ATOM | 7856 | CA | LYS | 4192 | 52.913 | −8.317 | 72.950 | 1.00 | 33.63 |
| ATOM | 7857 | CB | LYS | 4192 | 53.070 | −7.155 | 71.972 | 1.00 | 32.49 |
| ATOM | 7858 | CG | LYS | 4192 | 52.039 | −7.189 | 70.848 | 1.00 | 30.53 |
| ATOM | 7859 | CD | LYS | 4192 | 52.232 | −6.074 | 69.831 | 1.00 | 28.08 |
| ATOM | 7860 | CE | LYS | 4192 | 51.363 | −6.321 | 68.594 | 1.00 | 26.51 |
| ATOM | 7861 | NZ | LYS | 4192 | 51.408 | −5.234 | 67.570 | 1.00 | 25.16 |
| ATOM | 7862 | C | LYS | 4192 | 53.234 | −9.628 | 72.252 | 1.00 | 34.51 |
| ATOM | 7863 | O | LYS | 4192 | 54.296 | −9.772 | 71.654 | 1.00 | 34.65 |
| ATOM | 7864 | N | ASN | 4193 | 52.327 | −10.590 | 72.354 | 1.00 | 35.33 |
| ATOM | 7865 | CA | ASN | 4193 | 52.544 | −11.888 | 71.743 | 1.00 | 36.53 |
| ATOM | 7866 | CB | ASN | 4193 | 52.672 | −11.743 | 70.227 | 1.00 | 36.72 |
| ATOM | 7867 | CG | ASN | 4193 | 51.412 | −11.196 | 69.588 | 1.00 | 38.18 |
| ATOM | 7868 | OD1 | ASN | 4193 | 50.296 | −11.523 | 70.014 | 1.00 | 39.60 |
| ATOM | 7869 | ND2 | ASN | 4193 | 51.576 | −10.378 | 68.550 | 1.00 | 36.90 |
| ATOM | 7870 | C | ASN | 4193 | 53.798 | −12.565 | 72.304 | 1.00 | 37.39 |
| ATOM | 7871 | O | ASN | 4193 | 54.579 | −13.160 | 71.556 | 1.00 | 37.13 |
| ATOM | 7872 | N | GLY | 4194 | 53.987 | −12.459 | 73.620 | 1.00 | 38.08 |
| ATOM | 7873 | CA | GLY | 4194 | 55.126 | −13.091 | 74.263 | 1.00 | 38.95 |
| ATOM | 7874 | C | GLY | 4194 | 56.471 | −12.398 | 74.133 | 1.00 | 40.04 |
| ATOM | 7875 | O | GLY | 4194 | 57.423 | −12.761 | 74.823 | 1.00 | 39.97 |
| ATOM | 7876 | N | LYS | 4195 | 56.570 | −11.403 | 73.263 | 1.00 | 40.68 |
| ATOM | 7877 | CA | LYS | 4195 | 57.838 | −10.709 | 73.090 | 1.00 | 41.09 |
| ATOM | 7878 | CB | LYS | 4195 | 58.137 | −10.541 | 71.592 | 1.00 | 41.17 |
| ATOM | 7879 | C | LYS | 4195 | 57.848 | −9.355 | 73.798 | 1.00 | 41.53 |
| ATOM | 7880 | O | LYS | 4195 | 56.824 | −8.898 | 74.313 | 1.00 | 41.62 |
| ATOM | 7881 | N | GLU | 4196 | 59.014 | −8.720 | 73.838 | 1.00 | 42.10 |
| ATOM | 7882 | CA | GLU | 4196 | 59.125 | −7.417 | 74.475 | 1.00 | 43.11 |
| ATOM | 7883 | CB | GLU | 4196 | 60.592 | −6.955 | 74.533 | 1.00 | 44.94 |
| ATOM | 7884 | CG | GLU | 4196 | 60.746 | −5.461 | 74.862 | 1.00 | 47.89 |
| ATOM | 7885 | CD | GLU | 4196 | 62.182 | −5.036 | 75.152 | 1.00 | 49.90 |
| ATOM | 7886 | OE1 | GLU | 4196 | 63.112 | −5.552 | 74.488 | 1.00 | 51.03 |
| ATOM | 7887 | OE2 | GLU | 4196 | 62.380 | −4.169 | 76.036 | 1.00 | 50.90 |
| ATOM | 7888 | C | GLU | 4196 | 58.296 | −6.411 | 73.686 | 1.00 | 42.58 |
| ATOM | 7889 | O | GLU | 4196 | 58.244 | −6.467 | 72.458 | 1.00 | 43.01 |
| ATOM | 7890 | N | PHE | 4197 | 57.657 | −5.490 | 74.394 | 1.00 | 41.70 |
| ATOM | 7891 | CA | PHE | 4197 | 56.826 | −4.475 | 73.760 | 1.00 | 41.20 |
| ATOM | 7892 | CB | PHE | 4197 | 55.410 | −4.556 | 74.324 | 1.00 | 40.41 |
| ATOM | 7893 | CG | PHE | 4197 | 54.389 | −3.815 | 73.522 | 1.00 | 39.24 |
| ATOM | 7894 | CD1 | PHE | 4197 | 53.216 | −3.381 | 74.118 | 1.00 | 38.74 |
| ATOM | 7895 | CD2 | PHE | 4197 | 54.574 | −3.593 | 72.165 | 1.00 | 39.32 |
| ATOM | 7896 | CE1 | PHE | 4197 | 52.238 | −2.737 | 73.378 | 1.00 | 39.16 |
| ATOM | 7897 | CE2 | PHE | 4197 | 53.596 | −2.947 | 71.408 | 1.00 | 39.51 |
| ATOM | 7898 | CZ | PHE | 4197 | 52.425 | −2.520 | 72.019 | 1.00 | 39.30 |
| ATOM | 7899 | C | PHE | 4197 | 57.389 | −3.088 | 74.034 | 1.00 | 41.40 |
| ATOM | 7900 | O | PHE | 4197 | 57.455 | −2.662 | 75.177 | 1.00 | 41.46 |
| ATOM | 7901 | N | LYS | 4198 | 57.797 | −2.385 | 72.989 | 1.00 | 42.33 |
| ATOM | 7902 | CA | LYS | 4198 | 58.340 | −1.044 | 73.148 | 1.00 | 42.49 |
| ATOM | 7903 | CB | LYS | 4198 | 59.743 | −0.961 | 72.532 | 1.00 | 43.80 |
| ATOM | 7904 | CG | LYS | 4198 | 60.750 | −1.931 | 73.134 | 1.00 | 46.19 |
| ATOM | 7905 | CD | LYS | 4198 | 62.183 | −1.590 | 72.725 | 1.00 | 47.98 |
| ATOM | 7906 | CE | LYS | 4198 | 63.197 | −2.410 | 73.532 | 1.00 | 49.16 |
| ATOM | 7907 | NZ | LYS | 4198 | 64.621 | −1.994 | 73.312 | 1.00 | 49.73 |
| ATOM | 7908 | C | LYS | 4198 | 57.419 | −0.043 | 72.459 | 1.00 | 42.18 |
| ATOM | 7909 | O | LYS | 4198 | 56.866 | −0.326 | 71.394 | 1.00 | 41.91 |
| ATOM | 7910 | N | PRO | 4199 | 57.247 | 1.143 | 73.060 | 1.00 | 41.66 |
| ATOM | 7911 | CD | PRO | 4199 | 58.003 | 1.660 | 74.206 | 1.00 | 41.49 |
| ATOM | 7912 | CA | PRO | 4199 | 56.386 | 2.188 | 72.497 | 1.00 | 41.50 |
| ATOM | 7913 | CB | PRO | 4199 | 56.707 | 3.401 | 73.377 | 1.00 | 41.49 |
| ATOM | 7914 | CG | PRO | 4199 | 58.102 | 3.121 | 73.854 | 1.00 | 41.75 |
| ATOM | 7915 | C | PRO | 4199 | 56.611 | 2.448 | 71.007 | 1.00 | 41.24 |
| ATOM | 7916 | O | PRO | 4199 | 55.677 | 2.794 | 70.288 | 1.00 | 40.96 |
| ATOM | 7917 | N | ASP | 4200 | 57.839 | 2.270 | 70.542 | 1.00 | 41.42 |
| ATOM | 7918 | CA | ASP | 4200 | 58.151 | 2.492 | 69.127 | 1.00 | 41.34 |
| ATOM | 7919 | CB | ASP | 4200 | 59.669 | 2.472 | 68.892 | 1.00 | 42.51 |
| ATOM | 7920 | CG | ASP | 4200 | 60.294 | 3.867 | 68.858 | 1.00 | 44.82 |
| ATOM | 7921 | OD1 | ASP | 4200 | 59.567 | 4.879 | 68.685 | 1.00 | 45.34 |
| ATOM | 7922 | OD2 | ASP | 4200 | 61.540 | 3.947 | 68.984 | 1.00 | 46.30 |
| ATOM | 7923 | C | ASP | 4200 | 57.542 | 1.421 | 68.234 | 1.00 | 40.39 |
| ATOM | 7924 | O | ASP | 4200 | 57.636 | 1.508 | 67.010 | 1.00 | 40.23 |
| ATOM | 7925 | N | HIS | 4201 | 56.939 | 0.401 | 68.833 | 1.00 | 39.21 |
| ATOM | 7926 | CA | HIS | 4201 | 56.359 | −0.671 | 68.033 | 1.00 | 38.50 |
| ATOM | 7927 | CB | HIS | 4201 | 56.183 | −1.937 | 68.871 | 1.00 | 38.75 |
| ATOM | 7928 | CG | HIS | 4201 | 57.478 | −2.580 | 69.248 | 1.00 | 39.47 |
| ATOM | 7929 | CD2 | HIS | 4201 | 57.791 | −3.454 | 70.233 | 1.00 | 39.83 |
| ATOM | 7930 | ND1 | HIS | 4201 | 58.653 | −2.329 | 68.569 | 1.00 | 39.31 |
| ATOM | 7931 | CE1 | HIS | 4201 | 59.633 | −3.018 | 69.126 | 1.00 | 39.96 |
| ATOM | 7932 | NE2 | HIS | 4201 | 59.136 | −3.707 | 70.136 | 1.00 | 40.96 |
| ATOM | 7933 | C | HIS | 4201 | 55.046 | −0.290 | 67.374 | 1.00 | 37.52 |
| ATOM | 7934 | O | HIS | 4201 | 54.533 | −1.024 | 66.536 | 1.00 | 37.00 |
| ATOM | 7935 | N | ARG | 4202 | 54.495 | 0.856 | 67.752 | 1.00 | 36.56 |
| ATOM | 7936 | CA | ARG | 4202 | 53.260 | 1.311 | 67.137 | 1.00 | 35.61 |
| ATOM | 7937 | CB | ARG | 4202 | 52.041 | 0.843 | 67.942 | 1.00 | 34.07 |
| ATOM | 7938 | CG | ARG | 4202 | 51.782 | 1.561 | 69.239 | 1.00 | 31.36 |
| ATOM | 7939 | CD | ARG | 4202 | 50.716 | 0.817 | 70.017 | 1.00 | 30.34 |
| ATOM | 7940 | NE | ARG | 4202 | 49.425 | 0.750 | 69.334 | 1.00 | 27.90 |
| ATOM | 7941 | CZ | ARG | 4202 | 48.525 | 1.734 | 69.317 | 1.00 | 27.48 |
| ATOM | 7942 | NH1 | ARG | 4202 | 48.765 | 2.877 | 69.943 | 1.00 | 24.74 |
| ATOM | 7943 | NH2 | ARG | 4202 | 47.370 | 1.572 | 68.683 | 1.00 | 26.76 |
| ATOM | 7944 | C | ARG | 4202 | 53.284 | 2.824 | 67.033 | 1.00 | 35.89 |
| ATOM | 7945 | O | ARG | 4202 | 53.845 | 3.502 | 67.885 | 1.00 | 35.67 |
| ATOM | 7946 | N | ILE | 4203 | 52.690 | 3.358 | 65.974 | 1.00 | 36.51 |
| ATOM | 7947 | CA | ILE | 4203 | 52.671 | 4.795 | 65.806 | 1.00 | 37.34 |
| ATOM | 7948 | CB | ILE | 4203 | 52.034 | 5.198 | 64.458 | 1.00 | 37.30 |
| ATOM | 7949 | CG2 | ILE | 4203 | 50.558 | 4.925 | 64.470 | 1.00 | 38.05 |
| ATOM | 7950 | CG1 | ILE | 4203 | 52.251 | 6.689 | 64.188 | 1.00 | 38.22 |
| ATOM | 7951 | CD1 | ILE | 4203 | 53.670 | 7.065 | 63.839 | 1.00 | 37.31 |
| ATOM | 7952 | C | ILE | 4203 | 51.906 | 5.423 | 66.976 | 1.00 | 38.57 |
| ATOM | 7953 | O | ILE | 4203 | 50.781 | 5.023 | 67.319 | 1.00 | 39.04 |
| ATOM | 7954 | N | GLY | 4204 | 52.538 | 6.402 | 67.608 | 1.00 | 39.04 |
| ATOM | 7955 | CA | GLY | 4204 | 51.921 | 7.057 | 68.745 | 1.00 | 39.47 |
| ATOM | 7956 | C | GLY | 4204 | 52.291 | 6.365 | 70.047 | 1.00 | 39.68 |
| ATOM | 7957 | O | GLY | 4204 | 52.046 | 6.891 | 71.128 | 1.00 | 39.64 |
| ATOM | 7958 | N | GLY | 4205 | 52.883 | 5.180 | 69.938 | 1.00 | 39.30 |
| ATOM | 7959 | CA | GLY | 4205 | 53.271 | 4.436 | 71.115 | 1.00 | 38.53 |
| ATOM | 7960 | C | GLY | 4205 | 52.114 | 4.122 | 72.043 | 1.00 | 38.04 |
| ATOM | 7961 | O | GLY | 4205 | 50.966 | 4.031 | 71.629 | 1.00 | 38.21 |
| ATOM | 7962 | N | TYR | 4206 | 52.433 | 3.947 | 73.317 | 1.00 | 37.75 |
| ATOM | 7963 | CA | TYR | 4206 | 51.437 | 3.645 | 74.331 | 1.00 | 37.48 |
| ATOM | 7964 | CB | TYR | 4206 | 51.255 | 2.131 | 74.460 | 1.00 | 35.28 |
| ATOM | 7965 | CG | TYR | 4206 | 52.526 | 1.392 | 74.779 | 1.00 | 33.44 |
| ATOM | 7966 | CD1 | TYR | 4206 | 53.220 | 0.689 | 73.793 | 1.00 | 33.43 |
| ATOM | 7967 | CE1 | TYR | 4206 | 54.402 | 0.012 | 74.091 | 1.00 | 33.76 |
| ATOM | 7968 | CD2 | TYR | 4206 | 53.042 | 1.403 | 76.065 | 1.00 | 33.42 |
| ATOM | 7969 | CE2 | TYR | 4206 | 54.215 | 0.737 | 76.373 | 1.00 | 33.70 |
| ATOM | 7970 | CZ | TYR | 4206 | 54.893 | 0.045 | 75.389 | 1.00 | 33.58 |
| ATOM | 7971 | OH | TYR | 4206 | 56.068 | −0.584 | 75.725 | 1.00 | 33.58 |
| ATOM | 7972 | C | TYR | 4206 | 51.896 | 4.227 | 75.668 | 1.00 | 38.11 |
| ATOM | 7973 | O | TYR | 4206 | 53.037 | 4.651 | 75.807 | 1.00 | 37.77 |
| ATOM | 7974 | N | LYS | 4207 | 51.005 | 4.250 | 76.651 | 1.00 | 39.15 |
| ATOM | 7975 | CA | LYS | 4207 | 51.358 | 4.776 | 77.955 | 1.00 | 40.38 |
| ATOM | 7976 | CB | LYS | 4207 | 50.595 | 6.063 | 78.246 | 1.00 | 41.12 |
| ATOM | 7977 | CG | LYS | 4207 | 50.383 | 6.332 | 79.728 | 1.00 | 43.61 |
| ATOM | 7978 | CD | LYS | 4207 | 48.879 | 6.513 | 80.015 | 1.00 | 46.81 |
| ATOM | 7979 | CE | LYS | 4207 | 48.534 | 6.581 | 81.520 | 1.00 | 47.54 |
| ATOM | 7980 | NZ | LYS | 4207 | 47.102 | 6.959 | 81.783 | 1.00 | 47.13 |
| ATOM | 7981 | C | LYS | 4207 | 51.109 | 3.780 | 79.073 | 1.00 | 41.20 |
| ATOM | 7982 | O | LYS | 4207 | 50.051 | 3.154 | 79.156 | 1.00 | 41.62 |
| ATOM | 7983 | N | VAL | 4208 | 52.101 | 3.639 | 79.939 | 1.00 | 41.48 |
| ATOM | 7984 | CA | VAL | 4208 | 52.000 | 2.736 | 81.070 | 1.00 | 41.64 |
| ATOM | 7985 | CB | VAL | 4208 | 53.236 | 1.816 | 81.147 | 1.00 | 41.16 |
| ATOM | 7986 | CG1 | VAL | 4208 | 53.103 | 0.855 | 82.302 | 1.00 | 41.18 |
| ATOM | 7987 | CG2 | VAL | 4208 | 53.387 | 1.048 | 79.857 | 1.00 | 41.19 |
| ATOM | 7988 | C | VAL | 4208 | 51.913 | 3.562 | 82.351 | 1.00 | 42.21 |
| ATOM | 7989 | O | VAL | 4208 | 52.874 | 4.224 | 82.720 | 1.00 | 42.28 |
| ATOM | 7990 | N | ARG | 4209 | 50.754 | 3.559 | 83.005 | 1.00 | 42.95 |
| ATOM | 7991 | CA | ARG | 4209 | 50.609 | 4.293 | 84.257 | 1.00 | 43.59 |
| ATOM | 7992 | CB | ARG | 4209 | 49.243 | 4.992 | 84.344 | 1.00 | 44.90 |
| ATOM | 7993 | CG | ARG | 4209 | 49.057 | 5.876 | 85.596 | 1.00 | 47.55 |
| ATOM | 7994 | CD | ARG | 4209 | 50.115 | 6.999 | 85.700 | 1.00 | 50.42 |
| ATOM | 7995 | NE | ARG | 4209 | 49.543 | 8.350 | 85.560 | 1.00 | 52.46 |
| ATOM | 7996 | CZ | ARG | 4209 | 50.258 | 9.476 | 85.437 | 1.00 | 52.91 |
| ATOM | 7997 | NH1 | ARG | 4209 | 51.589 | 9.438 | 85.434 | 1.00 | 52.46 |
| ATOM | 7998 | NH2 | ARG | 4209 | 49.641 | 10.649 | 85.297 | 1.00 | 52.43 |
| ATOM | 7999 | C | ARG | 4209 | 50.760 | 3.238 | 85.348 | 1.00 | 43.36 |
| ATOM | 8000 | O | ARG | 4209 | 49.867 | 2.421 | 85.581 | 1.00 | 43.61 |
| ATOM | 8001 | N | TYR | 4210 | 51.911 | 3.242 | 86.007 | 1.00 | 42.62 |
| ATOM | 8002 | CA | TYR | 4210 | 52.173 | 2.261 | 87.041 | 1.00 | 41.53 |
| ATOM | 8003 | CB | TYR | 4210 | 53.628 | 2.336 | 87.489 | 1.00 | 41.84 |
| ATOM | 8004 | CG | TYR | 4210 | 54.593 | 2.047 | 86.364 | 1.00 | 42.55 |
| ATOM | 8005 | CD1 | TYR | 4210 | 54.887 | 3.019 | 85.401 | 1.00 | 41.88 |
| ATOM | 8006 | CE1 | TYR | 4210 | 55.745 | 2.750 | 84.344 | 1.00 | 41.89 |
| ATOM | 8007 | CD2 | TYR | 4210 | 55.183 | 0.789 | 86.236 | 1.00 | 42.54 |
| ATOM | 8008 | CE2 | TYR | 4210 | 56.043 | 0.505 | 85.174 | 1.00 | 43.32 |
| ATOM | 8009 | CZ | TYR | 4210 | 56.321 | 1.494 | 84.232 | 1.00 | 42.91 |
| ATOM | 8010 | OH | TYR | 4210 | 57.186 | 1.222 | 83.192 | 1.00 | 42.86 |
| ATOM | 8011 | C | TYR | 4210 | 51.270 | 2.359 | 88.240 | 1.00 | 41.29 |
| ATOM | 8012 | O | TYR | 4210 | 50.986 | 1.349 | 88.877 | 1.00 | 41.79 |
| ATOM | 8013 | N | ALA | 4211 | 50.810 | 3.564 | 88.551 | 1.00 | 40.80 |
| ATOM | 8014 | CA | ALA | 4211 | 49.938 | 3.748 | 89.703 | 1.00 | 40.57 |
| ATOM | 8015 | CB | ALA | 4211 | 49.610 | 5.219 | 89.884 | 1.00 | 40.78 |
| ATOM | 8016 | C | ALA | 4211 | 48.652 | 2.934 | 89.550 | 1.00 | 40.65 |
| ATOM | 8017 | O | ALA | 4211 | 48.080 | 2.467 | 90.539 | 1.00 | 40.67 |
| ATOM | 8018 | N | THR | 4212 | 48.200 | 2.766 | 88.311 | 1.00 | 40.06 |
| ATOM | 8019 | CA | THR | 4212 | 46.985 | 2.008 | 88.052 | 1.00 | 39.11 |
| ATOM | 8020 | CB | THR | 4212 | 45.984 | 2.796 | 87.160 | 1.00 | 39.65 |
| ATOM | 8021 | OG1 | THR | 4212 | 46.626 | 3.225 | 85.947 | 1.00 | 39.21 |
| ATOM | 8022 | CG2 | THR | 4212 | 45.443 | 4.000 | 87.915 | 1.00 | 39.45 |
| ATOM | 8023 | C | THR | 4212 | 47.265 | 0.672 | 87.387 | 1.00 | 38.62 |
| ATOM | 8024 | O | THR | 4212 | 46.342 | 0.000 | 86.939 | 1.00 | 38.56 |
| ATOM | 8025 | N | TRP | 4213 | 48.537 | 0.289 | 87.319 | 1.00 | 37.95 |
| ATOM | 8026 | CA | TRP | 4213 | 48.915 | −0.983 | 86.710 | 1.00 | 37.01 |
| ATOM | 8027 | CB | TRP | 4213 | 48.481 | −2.140 | 87.609 | 1.00 | 35.55 |
| ATOM | 8028 | CG | TRP | 4213 | 48.982 | −2.008 | 88.986 | 1.00 | 34.65 |
| ATOM | 8029 | CD2 | TRP | 4213 | 50.235 | −2.480 | 89.475 | 1.00 | 34.08 |
| ATOM | 8030 | CE2 | TRP | 4213 | 50.340 | −2.064 | 90.817 | 1.00 | 33.49 |
| ATOM | 8031 | CE3 | TRP | 4213 | 51.283 | −3.212 | 88.906 | 1.00 | 34.10 |
| ATOM | 8032 | CD1 | TRP | 4213 | 48.386 | −1.344 | 90.022 | 1.00 | 34.03 |
| ATOM | 8033 | NE1 | TRP | 4213 | 49.198 | −1.374 | 91.126 | 1.00 | 33.55 |
| ATOM | 8034 | CZ2 | TRP | 4213 | 51.453 | −2.356 | 91.601 | 1.00 | 33.61 |
| ATOM | 8035 | CZ3 | TRP | 4213 | 52.389 | −3.503 | 89.685 | 1.00 | 34.56 |
| ATOM | 8036 | CH2 | TRP | 4213 | 52.466 | −3.074 | 91.021 | 1.00 | 33.89 |
| ATOM | 8037 | C | TRP | 4213 | 48.249 | −1.142 | 85.356 | 1.00 | 36.65 |
| ATOM | 8038 | O | TRP | 4213 | 47.682 | −2.193 | 85.055 | 1.00 | 36.95 |
| ATOM | 8039 | N | SER | 4214 | 48.326 | −0.105 | 84.536 | 1.00 | 36.50 |
| ATOM | 8040 | CA | SER | 4214 | 47.679 | −0.137 | 83.231 | 1.00 | 36.33 |
| ATOM | 8041 | CB | SER | 4214 | 46.480 | 0.805 | 83.215 | 1.00 | 36.12 |
| ATOM | 8042 | OG | SER | 4214 | 45.602 | 0.543 | 84.287 | 1.00 | 39.08 |
| ATOM | 8043 | C | SER | 4214 | 48.555 | 0.262 | 82.070 | 1.00 | 35.75 |
| ATOM | 8044 | O | SER | 4214 | 49.588 | 0.910 | 82.240 | 1.00 | 36.25 |
| ATOM | 8045 | N | ILE | 4215 | 48.109 | −0.133 | 80.884 | 1.00 | 34.56 |
| ATOM | 8046 | CA | ILE | 4215 | 48.766 | 0.206 | 79.643 | 1.00 | 33.68 |
| ATOM | 8047 | CB | ILE | 4215 | 49.424 | −1.022 | 78.968 | 1.00 | 33.95 |
| ATOM | 8048 | CG2 | ILE | 4215 | 48.396 | −2.095 | 78.668 | 1.00 | 34.28 |
| ATOM | 8049 | CG1 | ILE | 4215 | 50.103 | −0.586 | 77.673 | 1.00 | 34.61 |
| ATOM | 8050 | CD1 | ILE | 4215 | 51.157 | −1.552 | 77.180 | 1.00 | 35.16 |
| ATOM | 8051 | C | ILE | 4215 | 47.636 | 0.754 | 78.787 | 1.00 | 33.46 |
| ATOM | 8052 | O | ILE | 4215 | 46.552 | 0.177 | 78.743 | 1.00 | 33.51 |
| ATOM | 8053 | N | ILE | 4216 | 47.879 | 1.890 | 78.140 | 1.00 | 33.41 |
| ATOM | 8054 | CA | ILE | 4216 | 46.866 | 2.533 | 77.309 | 1.00 | 33.11 |
| ATOM | 8055 | CB | ILE | 4216 | 46.575 | 3.992 | 77.762 | 1.00 | 32.84 |
| ATOM | 8056 | CG2 | ILE | 4216 | 45.296 | 4.482 | 77.117 | 1.00 | 31.62 |
| ATOM | 8057 | CG1 | ILE | 4216 | 46.442 | 4.085 | 79.279 | 1.00 | 33.97 |
| ATOM | 8058 | CD1 | ILE | 4216 | 45.094 | 3.629 | 79.824 | 1.00 | 35.66 |
| ATOM | 8059 | C | ILE | 4216 | 47.343 | 2.642 | 75.874 | 1.00 | 33.30 |
| ATOM | 8060 | O | ILE | 4216 | 48.452 | 3.123 | 75.626 | 1.00 | 33.41 |
| ATOM | 8061 | N | MSE | 4217 | 46.514 | 2.202 | 74.933 | 1.00 | 32.69 |
| ATOM | 8062 | CA | MSE | 4217 | 46.859 | 2.312 | 73.524 | 1.00 | 32.07 |
| ATOM | 8063 | CB | MSE | 4217 | 46.938 | 0.939 | 72.844 | 1.00 | 31.19 |
| ATOM | 8064 | CG | MSE | 4217 | 48.119 | 0.092 | 73.267 | 1.00 | 30.07 |
| ATOM | 8065 | SE | MSE | 4217 | 48.353 | −1.342 | 72.181 | 1.00 | 30.17 |
| ATOM | 8066 | CE | MSE | 4217 | 47.265 | −2.474 | 72.982 | 1.00 | 30.18 |
| ATOM | 8067 | C | MSE | 4217 | 45.760 | 3.140 | 72.886 | 1.00 | 32.49 |
| ATOM | 8068 | O | MSE | 4217 | 44.587 | 2.770 | 72.942 | 1.00 | 32.79 |
| ATOM | 8069 | N | ASP | 4218 | 46.133 | 4.278 | 72.312 | 1.00 | 32.42 |
| ATOM | 8070 | CA | ASP | 4218 | 45.150 | 5.120 | 71.660 | 1.00 | 32.43 |
| ATOM | 8071 | CB | ASP | 4218 | 45.549 | 6.587 | 71.720 | 1.00 | 34.71 |
| ATOM | 8072 | CG | ASP | 4218 | 44.965 | 7.298 | 72.922 | 1.00 | 37.11 |
| ATOM | 8073 | OD1 | ASP | 4218 | 44.051 | 6.733 | 73.565 | 1.00 | 39.13 |
| ATOM | 8074 | OD2 | ASP | 4218 | 45.405 | 8.429 | 73.216 | 1.00 | 37.76 |
| ATOM | 8075 | C | ASP | 4218 | 44.987 | 4.703 | 70.211 | 1.00 | 31.55 |
| ATOM | 8076 | O | ASP | 4218 | 45.926 | 4.215 | 69.583 | 1.00 | 31.19 |
| ATOM | 8077 | N | SER | 4219 | 43.774 | 4.875 | 69.701 | 1.00 | 30.56 |
| ATOM | 8078 | CA | SER | 4219 | 43.450 | 4.552 | 68.315 | 1.00 | 29.54 |
| ATOM | 8079 | CB | SER | 4219 | 43.989 | 5.667 | 67.409 | 1.00 | 28.99 |
| ATOM | 8080 | OG | SER | 4219 | 43.644 | 5.452 | 66.047 | 1.00 | 29.88 |
| ATOM | 8081 | C | SER | 4219 | 43.976 | 3.193 | 67.817 | 1.00 | 28.86 |
| ATOM | 8082 | O | SER | 4219 | 44.797 | 3.146 | 66.897 | 1.00 | 28.28 |
| ATOM | 8083 | N | VAL | 4220 | 43.503 | 2.089 | 68.388 | 1.00 | 27.68 |
| ATOM | 8084 | CA | VAL | 4220 | 44.009 | 0.794 | 67.935 | 1.00 | 27.42 |
| ATOM | 8085 | CB | VAL | 4220 | 43.547 | −0.408 | 68.833 | 1.00 | 26.40 |
| ATOM | 8086 | CG1 | VAL | 4220 | 43.912 | −0.144 | 70.271 | 1.00 | 26.34 |
| ATOM | 8087 | CG2 | VAL | 4220 | 42.053 | −0.657 | 68.689 | 1.00 | 26.39 |
| ATOM | 8088 | C | VAL | 4220 | 43.635 | 0.501 | 66.482 | 1.00 | 27.31 |
| ATOM | 8089 | O | VAL | 4220 | 42.620 | 0.969 | 65.975 | 1.00 | 27.45 |
| ATOM | 8090 | N | VAL | 4221 | 44.486 | −0.265 | 65.813 | 1.00 | 26.91 |
| ATOM | 8091 | CA | VAL | 4221 | 44.261 | −0.629 | 64.428 | 1.00 | 26.86 |
| ATOM | 8092 | CB | VAL | 4221 | 45.163 | 0.176 | 63.467 | 1.00 | 26.24 |
| ATOM | 8093 | CG1 | VAL | 4221 | 44.701 | 1.617 | 63.441 | 1.00 | 25.22 |
| ATOM | 8094 | CG2 | VAL | 4221 | 46.618 | 0.075 | 63.897 | 1.00 | 25.02 |
| ATOM | 8095 | C | VAL | 4221 | 44.541 | −2.109 | 64.293 | 1.00 | 27.45 |
| ATOM | 8096 | O | VAL | 4221 | 45.107 | −2.720 | 65.197 | 1.00 | 28.86 |
| ATOM | 8097 | N | PRO | 4222 | 44.156 | −2.711 | 63.163 | 1.00 | 27.42 |
| ATOM | 8098 | CD | PRO | 4222 | 43.557 | −2.110 | 61.963 | 1.00 | 28.16 |
| ATOM | 8099 | CA | PRO | 4222 | 44.377 | −4.144 | 62.950 | 1.00 | 27.83 |
| ATOM | 8100 | CB | PRO | 4222 | 44.110 | −4.322 | 61.460 | 1.00 | 27.16 |
| ATOM | 8101 | CG | PRO | 4222 | 43.057 | −3.335 | 61.208 | 1.00 | 27.80 |
| ATOM | 8102 | C | PRO | 4222 | 45.736 | −4.706 | 63.365 | 1.00 | 28.20 |
| ATOM | 8103 | O | PRO | 4222 | 45.799 | −5.823 | 63.874 | 1.00 | 28.54 |
| ATOM | 8104 | N | SER | 4223 | 46.813 | −3.948 | 63.156 | 1.00 | 28.13 |
| ATOM | 8105 | CA | SER | 4223 | 48.145 | −4.426 | 63.517 | 1.00 | 28.31 |
| ATOM | 8106 | CB | SER | 4223 | 49.220 | −3.546 | 62.872 | 1.00 | 28.40 |
| ATOM | 8107 | OG | SER | 4223 | 49.043 | −2.184 | 63.218 | 1.00 | 28.35 |
| ATOM | 8108 | C | SER | 4223 | 48.361 | −4.485 | 65.027 | 1.00 | 28.55 |
| ATOM | 8109 | O | SER | 4223 | 49.387 | −4.973 | 65.495 | 1.00 | 27.53 |
| ATOM | 8110 | N | ASP | 4224 | 47.405 | −3.981 | 65.797 | 1.00 | 29.04 |
| ATOM | 8111 | CA | ASP | 4224 | 47.556 | −4.049 | 67.239 | 1.00 | 29.81 |
| ATOM | 8112 | CB | ASP | 4224 | 46.916 | −2.843 | 67.910 | 1.00 | 30.46 |
| ATOM | 8113 | CG | ASP | 4224 | 47.563 | −1.554 | 67.488 | 1.00 | 32.25 |
| ATOM | 8114 | OD1 | ASP | 4224 | 48.815 | −1.535 | 67.379 | 1.00 | 33.00 |
| ATOM | 8115 | OD2 | ASP | 4224 | 46.829 | −0.566 | 67.264 | 1.00 | 32.98 |
| ATOM | 8116 | C | ASP | 4224 | 46.952 | −5.345 | 67.758 | 1.00 | 29.74 |
| ATOM | 8117 | O | ASP | 4224 | 47.104 | −5.683 | 68.923 | 1.00 | 29.28 |
| ATOM | 8118 | N | LYS | 4225 | 46.272 | −6.076 | 66.883 | 1.00 | 29.54 |
| ATOM | 8119 | CA | LYS | 4225 | 45.674 | −7.341 | 67.276 | 1.00 | 30.25 |
| ATOM | 8120 | CB | LYS | 4225 | 44.994 | −8.010 | 66.084 | 1.00 | 30.41 |
| ATOM | 8121 | CG | LYS | 4225 | 43.786 | −7.247 | 65.581 | 1.00 | 31.98 |
| ATOM | 8122 | CD | LYS | 4225 | 42.795 | −8.135 | 64.844 | 1.00 | 32.21 |
| ATOM | 8123 | CE | LYS | 4225 | 43.398 | −8.794 | 63.625 | 1.00 | 33.51 |
| ATOM | 8124 | NZ | LYS | 4225 | 42.323 | −9.233 | 62.696 | 1.00 | 34.72 |
| ATOM | 8125 | C | LYS | 4225 | 46.728 | −8.280 | 67.861 | 1.00 | 30.96 |
| ATOM | 8126 | O | LYS | 4225 | 47.854 | −8.355 | 67.368 | 1.00 | 31.27 |
| ATOM | 8127 | N | GLY | 4226 | 46.362 | −8.993 | 68.921 | 1.00 | 31.16 |
| ATOM | 8128 | CA | GLY | 4226 | 47.296 | −9.910 | 69.542 | 1.00 | 31.59 |
| ATOM | 8129 | C | GLY | 4226 | 47.051 | −10.141 | 71.023 | 1.00 | 32.15 |
| ATOM | 8130 | O | GLY | 4226 | 46.049 | −9.694 | 71.585 | 1.00 | 32.06 |
| ATOM | 8131 | N | ASN | 4227 | 47.970 | −10.859 | 71.661 | 1.00 | 32.44 |
| ATOM | 8132 | CA | ASN | 4227 | 47.845 | −11.129 | 73.080 | 1.00 | 32.63 |
| ATOM | 8133 | CB | ASN | 4227 | 48.180 | −12.586 | 73.397 | 1.00 | 31.91 |
| ATOM | 8134 | CG | ASN | 4227 | 47.153 | −13.546 | 72.828 | 1.00 | 33.37 |
| ATOM | 8135 | OD1 | ASN | 4227 | 45.950 | −13.374 | 73.028 | 1.00 | 34.47 |
| ATOM | 8136 | ND2 | ASN | 4227 | 47.620 | −14.563 | 72.112 | 1.00 | 33.39 |
| ATOM | 8137 | C | ASN | 4227 | 48.769 | −10.208 | 73.834 | 1.00 | 32.62 |
| ATOM | 8138 | O | ASN | 4227 | 49.920 | −10.013 | 73.459 | 1.00 | 33.21 |
| ATOM | 8139 | N | TYR | 4228 | 48.254 | −9.618 | 74.893 | 1.00 | 32.23 |
| ATOM | 8140 | CA | TYR | 4228 | 49.069 | −8.740 | 75.685 | 1.00 | 32.55 |
| ATOM | 8141 | CB | TYR | 4228 | 48.489 | −7.328 | 75.666 | 1.00 | 31.88 |
| ATOM | 8142 | CG | TYR | 4228 | 48.552 | −6.678 | 74.292 | 1.00 | 32.03 |
| ATOM | 8143 | CD1 | TYR | 4228 | 47.778 | −7.154 | 73.230 | 1.00 | 31.48 |
| ATOM | 8144 | CE1 | TYR | 4228 | 47.815 | −6.546 | 71.983 | 1.00 | 30.10 |
| ATOM | 8145 | CD2 | TYR | 4228 | 49.372 | −5.568 | 74.058 | 1.00 | 32.13 |
| ATOM | 8146 | CE2 | TYR | 4228 | 49.411 | −4.955 | 72.816 | 1.00 | 31.07 |
| ATOM | 8147 | CZ | TYR | 4228 | 48.629 | −5.450 | 71.783 | 1.00 | 30.54 |
| ATOM | 8148 | OH | TYR | 4228 | 48.678 | −4.844 | 70.550 | 1.00 | 30.38 |
| ATOM | 8149 | C | TYR | 4228 | 49.146 | −9.310 | 77.089 | 1.00 | 33.31 |
| ATOM | 8150 | O | TYR | 4228 | 48.129 | −9.551 | 77.746 | 1.00 | 33.33 |
| ATOM | 8151 | N | THR | 4229 | 50.376 | −9.555 | 77.526 | 1.00 | 34.33 |
| ATOM | 8152 | CA | THR | 4229 | 50.632 | −10.116 | 78.840 | 1.00 | 35.13 |
| ATOM | 8153 | CB | THR | 4229 | 51.509 | −11.352 | 78.752 | 1.00 | 34.68 |
| ATOM | 8154 | OG1 | THR | 4229 | 50.860 | −12.337 | 77.945 | 1.00 | 36.14 |
| ATOM | 8155 | CG2 | THR | 4229 | 51.765 | −11.910 | 80.135 | 1.00 | 34.19 |
| ATOM | 8156 | C | THR | 4229 | 51.338 | −9.148 | 79.762 | 1.00 | 36.17 |
| ATOM | 8157 | O | THR | 4229 | 52.315 | −8.512 | 79.382 | 1.00 | 37.31 |
| ATOM | 8158 | N | CYS | 4230 | 50.837 | −9.049 | 80.983 | 1.00 | 37.00 |
| ATOM | 8159 | CA | CYS | 4230 | 51.437 | −8.190 | 81.981 | 1.00 | 38.19 |
| ATOM | 8160 | CB | CYS | 4230 | 50.355 | −7.462 | 82.780 | 1.00 | 38.20 |
| ATOM | 8161 | SG | CYS | 4230 | 49.342 | −8.562 | 83.786 | 1.00 | 38.53 |
| ATOM | 8162 | C | CYS | 4230 | 52.218 | −9.115 | 82.911 | 1.00 | 39.00 |
| ATOM | 8163 | O | CYS | 4230 | 51.748 | −10.197 | 83.260 | 1.00 | 38.74 |
| ATOM | 8164 | N | ILE | 4231 | 53.411 | −8.687 | 83.299 | 1.00 | 40.04 |
| ATOM | 8165 | CA | ILE | 4231 | 54.255 | −9.461 | 84.192 | 1.00 | 40.81 |
| ATOM | 8166 | CB | ILE | 4231 | 55.582 | −9.815 | 83.533 | 1.00 | 40.38 |
| ATOM | 8167 | CG2 | ILE | 4231 | 56.494 | −10.478 | 84.540 | 1.00 | 39.21 |
| ATOM | 8168 | CG1 | ILE | 4231 | 55.327 | −10.714 | 82.329 | 1.00 | 40.35 |
| ATOM | 8169 | CD1 | ILE | 4231 | 56.577 | −11.069 | 81.564 | 1.00 | 40.79 |
| ATOM | 8170 | C | ILE | 4231 | 54.559 | −8.635 | 85.428 | 1.00 | 41.93 |
| ATOM | 8171 | O | ILE | 4231 | 55.290 | −7.647 | 85.354 | 1.00 | 41.40 |
| ATOM | 8172 | N | VAL | 4232 | 53.982 | −9.040 | 86.555 | 1.00 | 43.22 |
| ATOM | 8173 | CA | VAL | 4232 | 54.185 | −8.354 | 87.824 | 1.00 | 44.85 |
| ATOM | 8174 | CB | VAL | 4232 | 52.854 | −8.169 | 88.569 | 1.00 | 44.31 |
| ATOM | 8175 | CG1 | VAL | 4232 | 53.094 | −7.520 | 89.918 | 1.00 | 43.77 |
| ATOM | 8176 | CG2 | VAL | 4232 | 51.922 | −7.309 | 87.731 | 1.00 | 44.31 |
| ATOM | 8177 | C | VAL | 4232 | 55.128 | −9.189 | 88.675 | 1.00 | 46.31 |
| ATOM | 8178 | O | VAL | 4232 | 54.838 | −10.345 | 88.974 | 1.00 | 46.47 |
| ATOM | 8179 | N | GLU | 4233 | 56.257 | −8.611 | 89.070 | 1.00 | 47.92 |
| ATOM | 8180 | CA | GLU | 4233 | 57.215 | −9.368 | 89.853 | 1.00 | 49.67 |
| ATOM | 8181 | CB | GLU | 4233 | 58.094 | −10.187 | 88.899 | 1.00 | 50.76 |
| ATOM | 8182 | CG | GLU | 4233 | 58.834 | −9.351 | 87.858 | 1.00 | 54.07 |
| ATOM | 8183 | CD | GLU | 4233 | 59.401 | −10.176 | 86.693 | 1.00 | 55.52 |
| ATOM | 8184 | OE1 | GLU | 4233 | 59.888 | −11.306 | 86.940 | 1.00 | 56.69 |
| ATOM | 8185 | OE2 | GLU | 4233 | 59.369 | −9.682 | 85.535 | 1.00 | 55.80 |
| ATOM | 8186 | C | GLU | 4233 | 58.096 | −8.555 | 90.792 | 1.00 | 49.83 |
| ATOM | 8187 | O | GLU | 4233 | 58.290 | −7.354 | 90.611 | 1.00 | 49.22 |
| ATOM | 8188 | N | ASN | 4234 | 58.594 | −9.240 | 91.819 | 1.00 | 50.52 |
| ATOM | 8189 | CA | ASN | 4234 | 59.512 | −8.674 | 92.796 | 1.00 | 50.90 |
| ATOM | 8190 | CB | ASN | 4234 | 58.771 | −8.066 | 93.997 | 1.00 | 50.08 |
| ATOM | 8191 | CG | ASN | 4234 | 58.130 | −9.106 | 94.906 | 1.00 | 49.29 |
| ATOM | 8192 | OD1 | ASN | 4234 | 58.459 | −10.295 | 94.869 | 1.00 | 47.91 |
| ATOM | 8193 | ND2 | ASN | 4234 | 57.220 | −8.644 | 95.755 | 1.00 | 48.70 |
| ATOM | 8194 | C | ASN | 4234 | 60.419 | −9.832 | 93.211 | 1.00 | 52.10 |
| ATOM | 8195 | O | ASN | 4234 | 60.249 | −10.949 | 92.724 | 1.00 | 52.61 |
| ATOM | 8196 | N | GLU | 4235 | 61.379 | −9.575 | 94.093 | 1.00 | 53.29 |
| ATOM | 8197 | CA | GLU | 4235 | 62.324 | −10.607 | 94.537 | 1.00 | 53.78 |
| ATOM | 8198 | CB | GLU | 4235 | 63.187 | −10.070 | 95.683 | 1.00 | 54.71 |
| ATOM | 8199 | CG | GLU | 4235 | 63.910 | −8.765 | 95.387 | 1.00 | 57.27 |
| ATOM | 8200 | CD | GLU | 4235 | 64.385 | −8.058 | 96.657 | 1.00 | 58.65 |
| ATOM | 8201 | OE1 | GLU | 4235 | 63.516 | −7.673 | 97.481 | 1.00 | 59.03 |
| ATOM | 8202 | OE2 | GLU | 4235 | 65.617 | −7.888 | 96.829 | 1.00 | 58.24 |
| ATOM | 8203 | C | GLU | 4235 | 61.682 | −11.909 | 95.008 | 1.00 | 53.62 |
| ATOM | 8204 | O | GLU | 4235 | 62.287 | −12.977 | 94.913 | 1.00 | 53.31 |
| ATOM | 8205 | N | TYR | 4236 | 60.452 | −11.819 | 95.503 | 1.00 | 53.50 |
| ATOM | 8206 | CA | TYR | 4236 | 59.755 | −12.978 | 96.046 | 1.00 | 52.99 |
| ATOM | 8207 | CB | TYR | 4236 | 59.008 | −12.544 | 97.309 | 1.00 | 51.86 |
| ATOM | 8208 | CG | TYR | 4236 | 59.933 | −11.973 | 98.354 | 1.00 | 50.96 |
| ATOM | 8209 | CD1 | TYR | 4236 | 60.397 | −10.668 | 98.260 | 1.00 | 50.43 |
| ATOM | 8210 | CE1 | TYR | 4236 | 61.313 | −10.169 | 99.169 | 1.00 | 50.55 |
| ATOM | 8211 | CD2 | TYR | 4236 | 60.409 | −12.768 | 99.395 | 1.00 | 50.52 |
| ATOM | 8212 | CE2 | TYR | 4236 | 61.325 | −12.282 | 100.308 | 1.00 | 50.43 |
| ATOM | 8213 | CZ | TYR | 4236 | 61.774 | −10.984 | 100.191 | 1.00 | 50.72 |
| ATOM | 8214 | OH | TYR | 4236 | 62.692 | −10.503 | 101.097 | 1.00 | 50.94 |
| ATOM | 8215 | C | TYR | 4236 | 58.813 | −13.758 | 95.134 | 1.00 | 52.90 |
| ATOM | 8216 | O | TYR | 4236 | 58.279 | −14.799 | 95.531 | 1.00 | 53.18 |
| ATOM | 8217 | N | GLY | 4237 | 58.601 | −13.274 | 93.918 | 1.00 | 52.09 |
| ATOM | 8218 | CA | GLY | 4237 | 57.712 | −13.995 | 93.030 | 1.00 | 51.39 |
| ATOM | 8219 | C | GLY | 4237 | 57.208 | −13.191 | 91.857 | 1.00 | 50.83 |
| ATOM | 8220 | O | GLY | 4237 | 57.386 | −11.971 | 91.800 | 1.00 | 51.45 |
| ATOM | 8221 | N | SER | 4238 | 56.575 | −13.876 | 90.912 | 1.00 | 49.46 |
| ATOM | 8222 | CA | SER | 4238 | 56.046 | −13.204 | 89.739 | 1.00 | 48.52 |
| ATOM | 8223 | CB | SER | 4238 | 57.059 | −13.233 | 88.597 | 1.00 | 48.83 |
| ATOM | 8224 | OG | SER | 4238 | 56.831 | −14.350 | 87.766 | 1.00 | 50.56 |
| ATOM | 8225 | C | SER | 4238 | 54.744 | −13.824 | 89.264 | 1.00 | 47.25 |
| ATOM | 8226 | O | SER | 4238 | 54.583 | −15.043 | 89.247 | 1.00 | 47.33 |
| ATOM | 8227 | N | ILE | 4239 | 53.814 | −12.961 | 88.885 | 1.00 | 45.50 |
| ATOM | 8228 | CA | ILE | 4239 | 52.522 | −13.388 | 88.384 | 1.00 | 44.11 |
| ATOM | 8229 | CB | ILE | 4239 | 51.385 | −12.959 | 89.327 | 1.00 | 43.78 |
| ATOM | 8230 | CG2 | ILE | 4239 | 51.587 | −13.611 | 90.685 | 1.00 | 43.69 |
| ATOM | 8231 | CG1 | ILE | 4239 | 51.336 | −11.432 | 89.444 | 1.00 | 42.04 |
| ATOM | 8232 | CD1 | ILE | 4239 | 50.270 | −10.930 | 90.393 | 1.00 | 41.89 |
| ATOM | 8233 | C | ILE | 4239 | 52.303 | −12.751 | 87.019 | 1.00 | 43.59 |
| ATOM | 8234 | O | ILE | 4239 | 53.037 | −11.850 | 86.617 | 1.00 | 43.53 |
| ATOM | 8235 | N | ASN | 4240 | 51.295 | −13.222 | 86.300 | 1.00 | 43.01 |
| ATOM | 8236 | CA | ASN | 4240 | 51.006 | −12.674 | 84.987 | 1.00 | 42.09 |
| ATOM | 8237 | CB | ASN | 4240 | 51.998 | −13.209 | 83.973 | 1.00 | 41.85 |
| ATOM | 8238 | CG | ASN | 4240 | 52.059 | −14.707 | 83.973 | 1.00 | 41.98 |
| ATOM | 8239 | OD1 | ASN | 4240 | 51.113 | −15.382 | 83.572 | 1.00 | 42.56 |
| ATOM | 8240 | ND2 | ASN | 4240 | 53.170 | −15.244 | 84.436 | 1.00 | 42.67 |
| ATOM | 8241 | C | ASN | 4240 | 49.596 | −12.991 | 84.531 | 1.00 | 41.72 |
| ATOM | 8242 | O | ASN | 4240 | 48.939 | −13.887 | 85.061 | 1.00 | 40.95 |
| ATOM | 8243 | N | HIS | 4241 | 49.142 | −12.237 | 83.538 | 1.00 | 41.41 |
| ATOM | 8244 | CA | HIS | 4241 | 47.811 | −12.403 | 82.983 | 1.00 | 40.60 |
| ATOM | 8245 | CB | HIS | 4241 | 46.825 | −11.496 | 83.705 | 1.00 | 41.26 |
| ATOM | 8246 | CG | HIS | 4241 | 45.393 | −11.818 | 83.417 | 1.00 | 43.06 |
| ATOM | 8247 | CD2 | HIS | 4241 | 44.484 | −11.209 | 82.618 | 1.00 | 42.94 |
| ATOM | 8248 | ND1 | HIS | 4241 | 44.745 | −12.895 | 83.983 | 1.00 | 43.33 |
| ATOM | 8249 | CE1 | HIS | 4241 | 43.497 | −12.934 | 83.549 | 1.00 | 43.09 |
| ATOM | 8250 | NE2 | HIS | 4241 | 43.314 | −11.923 | 82.719 | 1.00 | 43.49 |
| ATOM | 8251 | C | HIS | 4241 | 47.895 | −11.994 | 81.528 | 1.00 | 40.25 |
| ATOM | 8252 | O | HIS | 4241 | 48.701 | −11.137 | 81.172 | 1.00 | 40.67 |
| ATOM | 8253 | N | THR | 4242 | 47.075 | −12.606 | 80.682 | 1.00 | 39.24 |
| ATOM | 8254 | CA | THR | 4242 | 47.087 | −12.277 | 79.266 | 1.00 | 37.65 |
| ATOM | 8255 | CB | THR | 4242 | 47.631 | −13.431 | 78.433 | 1.00 | 37.75 |
| ATOM | 8256 | OG1 | THR | 4242 | 48.930 | −13.791 | 78.913 | 1.00 | 38.35 |
| ATOM | 8257 | CG2 | THR | 4242 | 47.729 | −13.023 | 76.969 | 1.00 | 37.80 |
| ATOM | 8258 | C | THR | 4242 | 45.719 | −11.923 | 78.725 | 1.00 | 37.28 |
| ATOM | 8259 | O | THR | 4242 | 44.745 | −12.640 | 78.938 | 1.00 | 37.57 |
| ATOM | 8260 | N | TYR | 4243 | 45.655 | −10.804 | 78.018 | 1.00 | 36.34 |
| ATOM | 8261 | CA | TYR | 4243 | 44.412 | −10.357 | 77.422 | 1.00 | 35.42 |
| ATOM | 8262 | CB | TYR | 4243 | 44.175 | −8.896 | 77.755 | 1.00 | 34.73 |
| ATOM | 8263 | CG | TYR | 4243 | 44.004 | −8.612 | 79.216 | 1.00 | 33.78 |
| ATOM | 8264 | CD1 | TYR | 4243 | 44.958 | −7.888 | 79.914 | 1.00 | 33.62 |
| ATOM | 8265 | CE1 | TYR | 4243 | 44.761 | −7.524 | 81.244 | 1.00 | 33.55 |
| ATOM | 8266 | CD2 | TYR | 4243 | 42.844 | −8.987 | 79.884 | 1.00 | 34.18 |
| ATOM | 8267 | CE2 | TYR | 4243 | 42.635 | −8.634 | 81.212 | 1.00 | 33.61 |
| ATOM | 8268 | CZ | TYR | 4243 | 43.599 | −7.896 | 81.884 | 1.00 | 33.75 |
| ATOM | 8269 | OH | TYR | 4243 | 43.389 | −7.503 | 83.184 | 1.00 | 34.63 |
| ATOM | 8270 | C | TYR | 4243 | 44.530 | −10.502 | 75.919 | 1.00 | 35.25 |
| ATOM | 8271 | O | TYR | 4243 | 45.626 | −10.405 | 75.366 | 1.00 | 35.67 |
| ATOM | 8272 | N | GLN | 4244 | 43.416 | −10.752 | 75.248 | 1.00 | 35.17 |
| ATOM | 8273 | CA | GLN | 4244 | 43.470 | −10.855 | 73.802 | 1.00 | 34.91 |
| ATOM | 8274 | CB | GLN | 4244 | 42.784 | −12.123 | 73.288 | 1.00 | 36.77 |
| ATOM | 8275 | CG | GLN | 4244 | 42.980 | −12.299 | 71.770 | 1.00 | 40.07 |
| ATOM | 8276 | CD | GLN | 4244 | 42.248 | −13.502 | 71.181 | 1.00 | 42.27 |
| ATOM | 8277 | OE1 | GLN | 4244 | 41.644 | −14.305 | 71.908 | 1.00 | 44.10 |
| ATOM | 8278 | NE2 | GLN | 4244 | 42.302 | −13.637 | 69.857 | 1.00 | 42.29 |
| ATOM | 8279 | C | GLN | 4244 | 42.789 | −9.631 | 73.231 | 1.00 | 33.75 |
| ATOM | 8280 | O | GLN | 4244 | 41.683 | −9.278 | 73.638 | 1.00 | 33.38 |
| ATOM | 8281 | N | LEU | 4245 | 43.471 | −8.968 | 72.309 | 1.00 | 32.82 |
| ATOM | 8282 | CA | LEU | 4245 | 42.932 | −7.780 | 71.681 | 1.00 | 32.23 |
| ATOM | 8283 | CB | LEU | 4245 | 43.960 | −6.650 | 71.709 | 1.00 | 32.31 |
| ATOM | 8284 | CG | LEU | 4245 | 43.625 | −5.419 | 70.849 | 1.00 | 33.45 |
| ATOM | 8285 | CD1 | LEU | 4245 | 42.283 | −4.840 | 71.259 | 1.00 | 32.44 |
| ATOM | 8286 | CD2 | LEU | 4245 | 44.734 | −4.371 | 70.990 | 1.00 | 33.75 |
| ATOM | 8287 | C | LEU | 4245 | 42.540 | −8.075 | 70.246 | 1.00 | 31.65 |
| ATOM | 8288 | O | LEU | 4245 | 43.340 | −8.569 | 69.452 | 1.00 | 31.23 |
| ATOM | 8289 | N | ASP | 4246 | 41.292 | −7.776 | 69.923 | 1.00 | 31.78 |
| ATOM | 8290 | CA | ASP | 4246 | 40.782 | −7.983 | 68.579 | 1.00 | 31.60 |
| ATOM | 8291 | CB | ASP | 4246 | 39.709 | −9.063 | 68.595 | 1.00 | 31.28 |
| ATOM | 8292 | CG | ASP | 4246 | 39.308 | −9.500 | 67.210 | 1.00 | 31.57 |
| ATOM | 8293 | OD1 | ASP | 4246 | 38.394 | −10.347 | 67.114 | 1.00 | 31.79 |
| ATOM | 8294 | OD2 | ASP | 4246 | 39.899 | −9.005 | 66.221 | 1.00 | 29.97 |
| ATOM | 8295 | C | ASP | 4246 | 40.195 | −6.665 | 68.075 | 1.00 | 31.32 |
| ATOM | 8296 | O | ASP | 4246 | 39.348 | −6.066 | 68.737 | 1.00 | 31.34 |
| ATOM | 8297 | N | VAL | 4247 | 40.667 | −6.203 | 66.921 | 1.00 | 30.94 |
| ATOM | 8298 | CA | VAL | 4247 | 40.178 | −4.957 | 66.341 | 1.00 | 30.64 |
| ATOM | 8299 | CB | VAL | 4247 | 41.336 | −4.007 | 65.953 | 1.00 | 29.76 |
| ATOM | 8300 | CG1 | VAL | 4247 | 40.794 | −2.759 | 65.302 | 1.00 | 28.69 |
| ATOM | 8301 | CG2 | VAL | 4247 | 42.135 | −3.645 | 67.170 | 1.00 | 29.66 |
| ATOM | 8302 | C | VAL | 4247 | 39.378 | −5.282 | 65.084 | 1.00 | 31.83 |
| ATOM | 8303 | O | VAL | 4247 | 39.862 | −5.990 | 64.197 | 1.00 | 32.23 |
| ATOM | 8304 | N | VAL | 4248 | 38.156 | −4.757 | 65.010 | 1.00 | 32.26 |
| ATOM | 8305 | CA | VAL | 4248 | 37.264 | −4.989 | 63.867 | 1.00 | 32.81 |
| ATOM | 8306 | CB | VAL | 4248 | 35.873 | −5.501 | 64.340 | 1.00 | 32.43 |
| ATOM | 8307 | CG1 | VAL | 4248 | 34.888 | −5.492 | 63.190 | 1.00 | 32.46 |
| ATOM | 8308 | CG2 | VAL | 4248 | 35.990 | −6.896 | 64.891 | 1.00 | 33.47 |
| ATOM | 8309 | C | VAL | 4248 | 37.019 | −3.693 | 63.081 | 1.00 | 33.17 |
| ATOM | 8310 | O | VAL | 4248 | 36.651 | −2.689 | 63.660 | 1.00 | 33.40 |
| ATOM | 8311 | N | GLU | 4249 | 37.182 | −3.679 | 61.768 | 1.00 | 33.84 |
| ATOM | 8312 | CA | GLU | 4249 | 36.924 | −2.425 | 61.045 | 1.00 | 34.40 |
| ATOM | 8313 | CB | GLU | 4249 | 37.877 | −2.309 | 59.847 | 1.00 | 34.83 |
| ATOM | 8314 | CG | GLU | 4249 | 39.344 | −2.426 | 60.256 | 1.00 | 36.55 |
| ATOM | 8315 | CD | GLU | 4249 | 40.318 | −2.056 | 59.150 | 1.00 | 39.13 |
| ATOM | 8316 | OE1 | GLU | 4249 | 40.634 | −2.920 | 58.297 | 1.00 | 40.64 |
| ATOM | 8317 | OE2 | GLU | 4249 | 40.771 | −0.888 | 59.130 | 1.00 | 39.71 |
| ATOM | 8318 | C | GLU | 4249 | 35.458 | −2.320 | 60.596 | 1.00 | 33.65 |
| ATOM | 8319 | O | GLU | 4249 | 34.837 | −3.333 | 60.278 | 1.00 | 34.15 |
| ATOM | 8320 | N | ARG | 4250 | 34.899 | −1.109 | 60.599 | 1.00 | 32.84 |
| ATOM | 8321 | CA | ARG | 4250 | 33.504 | −0.914 | 60.182 | 1.00 | 33.16 |
| ATOM | 8322 | CB | ARG | 4250 | 32.709 | −0.118 | 61.232 | 1.00 | 32.28 |
| ATOM | 8323 | CG | ARG | 4250 | 32.700 | −0.684 | 62.657 | 1.00 | 31.28 |
| ATOM | 8324 | CD | ARG | 4250 | 32.366 | −2.166 | 62.713 | 1.00 | 30.77 |
| ATOM | 8325 | NE | ARG | 4250 | 31.024 | −2.499 | 62.245 | 1.00 | 30.24 |
| ATOM | 8326 | CZ | ARG | 4250 | 29.912 | −2.359 | 62.962 | 1.00 | 31.10 |
| ATOM | 8327 | NH1 | ARG | 4250 | 29.965 | −1.883 | 64.196 | 1.00 | 31.16 |
| ATOM | 8328 | NH2 | ARG | 4250 | 28.743 | −2.718 | 62.451 | 1.00 | 31.07 |
| ATOM | 8329 | C | ARG | 4250 | 33.414 | −0.178 | 58.834 | 1.00 | 34.09 |
| ATOM | 8330 | O | ARG | 4250 | 34.352 | 0.514 | 58.434 | 1.00 | 34.30 |
| ATOM | 8331 | N | SER | 4251 | 32.287 | −0.341 | 58.141 | 1.00 | 34.95 |
| ATOM | 8332 | CA | SER | 4251 | 32.043 | 0.304 | 56.846 | 1.00 | 35.73 |
| ATOM | 8333 | CB | SER | 4251 | 32.136 | −0.706 | 55.709 | 1.00 | 35.29 |
| ATOM | 8334 | OG | SER | 4251 | 33.432 | −1.272 | 55.655 | 1.00 | 38.10 |
| ATOM | 8335 | C | SER | 4251 | 30.653 | 0.918 | 56.827 | 1.00 | 35.91 |
| ATOM | 8336 | O | SER | 4251 | 29.698 | 0.288 | 56.389 | 1.00 | 36.05 |
| ATOM | 8337 | N | PRO | 4252 | 30.529 | 2.165 | 57.298 | 1.00 | 36.23 |
| ATOM | 8338 | CD | PRO | 4252 | 31.640 | 2.988 | 57.810 | 1.00 | 35.96 |
| ATOM | 8339 | CA | PRO | 4252 | 29.266 | 2.907 | 57.358 | 1.00 | 36.03 |
| ATOM | 8340 | CB | PRO | 4252 | 29.559 | 3.964 | 58.411 | 1.00 | 35.84 |
| ATOM | 8341 | CG | PRO | 4252 | 30.962 | 4.337 | 58.067 | 1.00 | 36.40 |
| ATOM | 8342 | C | PRO | 4252 | 28.885 | 3.520 | 56.011 | 1.00 | 36.05 |
| ATOM | 8343 | O | PRO | 4252 | 28.773 | 4.739 | 55.877 | 1.00 | 36.52 |
| ATOM | 8344 | N | HIS | 4253 | 28.691 | 2.665 | 55.016 | 1.00 | 35.94 |
| ATOM | 8345 | CA | HIS | 4253 | 28.328 | 3.093 | 53.666 | 1.00 | 35.62 |
| ATOM | 8346 | CB | HIS | 4253 | 29.478 | 2.884 | 52.679 | 1.00 | 38.03 |
| ATOM | 8347 | CG | HIS | 4253 | 30.715 | 3.657 | 52.996 | 1.00 | 42.01 |
| ATOM | 8348 | CD2 | HIS | 4253 | 31.282 | 4.720 | 52.377 | 1.00 | 43.13 |
| ATOM | 8349 | ND1 | HIS | 4253 | 31.542 | 3.340 | 54.054 | 1.00 | 44.01 |
| ATOM | 8350 | CE1 | HIS | 4253 | 32.567 | 4.176 | 54.071 | 1.00 | 44.30 |
| ATOM | 8351 | NE2 | HIS | 4253 | 32.434 | 5.022 | 53.064 | 1.00 | 44.43 |
| ATOM | 8352 | C | HIS | 4253 | 27.195 | 2.233 | 53.154 | 1.00 | 34.14 |
| ATOM | 8353 | O | HIS | 4253 | 26.943 | 1.150 | 53.674 | 1.00 | 33.82 |
| ATOM | 8354 | N | ARG | 4254 | 26.539 | 2.702 | 52.101 | 1.00 | 32.61 |
| ATOM | 8355 | CA | ARG | 4254 | 25.465 | 1.931 | 51.508 | 1.00 | 31.56 |
| ATOM | 8356 | CB | ARG | 4254 | 24.775 | 2.743 | 50.410 | 1.00 | 31.74 |
| ATOM | 8357 | CG | ARG | 4254 | 25.624 | 3.051 | 49.205 | 1.00 | 31.47 |
| ATOM | 8358 | CD | ARG | 4254 | 24.852 | 3.991 | 48.329 | 1.00 | 33.11 |
| ATOM | 8359 | NE | ARG | 4254 | 25.081 | 3.769 | 46.907 | 1.00 | 36.52 |
| ATOM | 8360 | CZ | ARG | 4254 | 26.002 | 4.402 | 46.190 | 1.00 | 37.94 |
| ATOM | 8361 | NH1 | ARG | 4254 | 26.787 | 5.299 | 46.773 | 1.00 | 38.95 |
| ATOM | 8362 | NH2 | ARG | 4254 | 26.126 | 4.153 | 44.890 | 1.00 | 38.46 |
| ATOM | 8363 | C | ARG | 4254 | 26.096 | 0.662 | 50.940 | 1.00 | 30.31 |
| ATOM | 8364 | O | ARG | 4254 | 27.314 | 0.579 | 50.789 | 1.00 | 30.82 |
| ATOM | 8365 | N | PRO | 4255 | 25.282 | −0.356 | 50.631 | 1.00 | 29.32 |
| ATOM | 8366 | CD | PRO | 4255 | 23.849 | −0.548 | 50.916 | 1.00 | 28.41 |
| ATOM | 8367 | CA | PRO | 4255 | 25.897 | −1.572 | 50.095 | 1.00 | 28.37 |
| ATOM | 8368 | CB | PRO | 4255 | 24.727 | −2.559 | 49.995 | 1.00 | 27.41 |
| ATOM | 8369 | CG | PRO | 4255 | 23.505 | −1.686 | 50.003 | 1.00 | 28.78 |
| ATOM | 8370 | C | PRO | 4255 | 26.643 | −1.352 | 48.783 | 1.00 | 28.66 |
| ATOM | 8371 | O | PRO | 4255 | 26.349 | −0.427 | 48.032 | 1.00 | 28.94 |
| ATOM | 8372 | N | ILE | 4256 | 27.635 | −2.201 | 48.542 | 1.00 | 28.54 |
| ATOM | 8373 | CA | ILE | 4256 | 28.470 | −2.137 | 47.356 | 1.00 | 28.76 |
| ATOM | 8374 | CB | ILE | 4256 | 29.957 | −2.104 | 47.745 | 1.00 | 29.42 |
| ATOM | 8375 | CG2 | ILE | 4256 | 30.822 | −2.187 | 46.480 | 1.00 | 28.73 |
| ATOM | 8376 | CG1 | ILE | 4256 | 30.261 | −0.879 | 48.598 | 1.00 | 28.65 |
| ATOM | 8377 | CD1 | ILE | 4256 | 31.685 | −0.849 | 49.094 | 1.00 | 28.28 |
| ATOM | 8378 | C | ILE | 4256 | 28.278 | −3.407 | 46.547 | 1.00 | 29.44 |
| ATOM | 8379 | O | ILE | 4256 | 28.437 | −4.499 | 47.092 | 1.00 | 29.25 |
| ATOM | 8380 | N | LEU | 4257 | 27.961 | −3.279 | 45.258 | 1.00 | 29.59 |
| ATOM | 8381 | CA | LEU | 4257 | 27.772 | −4.462 | 44.413 | 1.00 | 30.28 |
| ATOM | 8382 | CB | LEU | 4257 | 26.526 | −4.298 | 43.538 | 1.00 | 31.83 |
| ATOM | 8383 | CG | LEU | 4257 | 25.233 | −3.896 | 44.256 | 1.00 | 31.86 |
| ATOM | 8384 | CD1 | LEU | 4257 | 24.043 | −4.028 | 43.318 | 1.00 | 32.53 |
| ATOM | 8385 | CD2 | LEU | 4257 | 25.026 | −4.777 | 45.453 | 1.00 | 32.23 |
| ATOM | 8386 | C | LEU | 4257 | 29.019 | −4.649 | 43.537 | 1.00 | 31.29 |
| ATOM | 8387 | O | LEU | 4257 | 29.557 | −3.670 | 43.008 | 1.00 | 31.75 |
| ATOM | 8388 | N | GLN | 4258 | 29.485 | −5.893 | 43.387 | 1.00 | 31.31 |
| ATOM | 8389 | CA | GLN | 4258 | 30.689 | −6.085 | 42.590 | 1.00 | 32.13 |
| ATOM | 8390 | CB | GLN | 4258 | 31.258 | −7.500 | 42.667 | 1.00 | 33.85 |
| ATOM | 8391 | CG | GLN | 4258 | 32.533 | −7.669 | 41.794 | 1.00 | 35.66 |
| ATOM | 8392 | CD | GLN | 4258 | 33.682 | −6.690 | 42.145 | 1.00 | 37.42 |
| ATOM | 8393 | OE1 | GLN | 4258 | 33.453 | −5.653 | 42.767 | 1.00 | 39.34 |
| ATOM | 8394 | NE2 | GLN | 4258 | 34.911 | −7.014 | 41.726 | 1.00 | 36.97 |
| ATOM | 8395 | C | GLN | 4258 | 30.474 | −5.740 | 41.134 | 1.00 | 32.61 |
| ATOM | 8396 | O | GLN | 4258 | 29.553 | −6.232 | 40.489 | 1.00 | 32.50 |
| ATOM | 8397 | N | ALA | 4259 | 31.356 | −4.884 | 40.623 | 1.00 | 32.68 |
| ATOM | 8398 | CA | ALA | 4259 | 31.281 | −4.421 | 39.257 | 1.00 | 32.67 |
| ATOM | 8399 | CB | ALA | 4259 | 32.504 | −3.569 | 38.879 | 1.00 | 31.94 |
| ATOM | 8400 | C | ALA | 4259 | 31.156 | −5.607 | 38.317 | 1.00 | 32.89 |
| ATOM | 8401 | O | ALA | 4259 | 31.771 | −6.663 | 38.534 | 1.00 | 32.92 |
| ATOM | 8402 | N | GLY | 4260 | 30.342 | −5.441 | 37.277 | 1.00 | 32.68 |
| ATOM | 8403 | CA | GLY | 4260 | 30.175 | −6.506 | 36.309 | 1.00 | 32.23 |
| ATOM | 8404 | C | GLY | 4260 | 29.040 | −7.483 | 36.534 | 1.00 | 32.64 |
| ATOM | 8405 | O | GLY | 4260 | 28.693 | −8.221 | 35.620 | 1.00 | 33.72 |
| ATOM | 8406 | N | LEU | 4261 | 28.454 | −7.511 | 37.725 | 1.00 | 32.98 |
| ATOM | 8407 | CA | LEU | 4261 | 27.360 | −8.445 | 37.977 | 1.00 | 32.81 |
| ATOM | 8408 | CB | LEU | 4261 | 27.796 | −9.506 | 38.984 | 1.00 | 33.61 |
| ATOM | 8409 | CG | LEU | 4261 | 29.094 | −10.234 | 38.626 | 1.00 | 34.82 |
| ATOM | 8410 | CD1 | LEU | 4261 | 29.573 | −11.062 | 39.816 | 1.00 | 35.54 |
| ATOM | 8411 | CD2 | LEU | 4261 | 28.875 | −11.112 | 37.398 | 1.00 | 34.63 |
| ATOM | 8412 | C | LEU | 4261 | 26.107 | −7.737 | 38.479 | 1.00 | 33.03 |
| ATOM | 8413 | O | LEU | 4261 | 26.192 | −6.814 | 39.294 | 1.00 | 32.77 |
| ATOM | 8414 | N | PRO | 4262 | 24.922 | −8.160 | 37.988 | 1.00 | 32.72 |
| ATOM | 8415 | CD | PRO | 4262 | 23.617 | −7.607 | 38.392 | 1.00 | 32.23 |
| ATOM | 8416 | CA | PRO | 4262 | 24.745 | −9.245 | 37.005 | 1.00 | 32.53 |
| ATOM | 8417 | CB | PRO | 4262 | 23.237 | −9.469 | 36.999 | 1.00 | 32.39 |
| ATOM | 8418 | CG | PRO | 4262 | 22.707 | −8.098 | 37.274 | 1.00 | 32.77 |
| ATOM | 8419 | C | PRO | 4262 | 25.256 | −8.848 | 35.632 | 1.00 | 32.65 |
| ATOM | 8420 | O | PRO | 4262 | 25.415 | −7.666 | 35.342 | 1.00 | 33.33 |
| ATOM | 8421 | N | ALA | 4263 | 25.499 | −9.834 | 34.781 | 1.00 | 33.35 |
| ATOM | 8422 | CA | ALA | 4263 | 26.001 | −9.557 | 33.443 | 1.00 | 33.60 |
| ATOM | 8423 | CB | ALA | 4263 | 27.245 | −10.373 | 33.180 | 1.00 | 33.10 |
| ATOM | 8424 | C | ALA | 4263 | 24.961 | −9.833 | 32.366 | 1.00 | 34.13 |
| ATOM | 8425 | O | ALA | 4263 | 24.065 | −10.659 | 32.552 | 1.00 | 33.73 |
| ATOM | 8426 | N | ASN | 4264 | 25.075 | −9.122 | 31.246 | 1.00 | 35.01 |
| ATOM | 8427 | CA | ASN | 4264 | 24.139 | −9.302 | 30.136 | 1.00 | 36.56 |
| ATOM | 8428 | CB | ASN | 4264 | 24.435 | −8.318 | 28.998 | 1.00 | 36.07 |
| ATOM | 8429 | CG | ASN | 4264 | 23.999 | −6.906 | 29.328 | 1.00 | 36.15 |
| ATOM | 8430 | OD1 | ASN | 4264 | 23.048 | −6.705 | 30.093 | 1.00 | 36.46 |
| ATOM | 8431 | ND2 | ASN | 4264 | 24.675 | −5.916 | 28.742 | 1.00 | 34.85 |
| ATOM | 8432 | C | ASN | 4264 | 24.249 | −10.723 | 29.617 | 1.00 | 37.84 |
| ATOM | 8433 | O | ASN | 4264 | 25.337 | −11.295 | 29.570 | 1.00 | 38.10 |
| ATOM | 8434 | N | LYS | 4265 | 23.119 | −11.295 | 29.223 | 1.00 | 39.00 |
| ATOM | 8435 | CA | LYS | 4265 | 23.102 | −12.662 | 28.717 | 1.00 | 40.10 |
| ATOM | 8436 | CB | LYS | 4265 | 22.514 | −13.605 | 29.763 | 1.00 | 41.33 |
| ATOM | 8437 | CG | LYS | 4265 | 23.129 | −13.512 | 31.132 | 1.00 | 41.86 |
| ATOM | 8438 | CD | LYS | 4265 | 24.322 | −14.422 | 31.272 | 1.00 | 42.16 |
| ATOM | 8439 | CE | LYS | 4265 | 24.617 | −14.611 | 32.744 | 1.00 | 42.74 |
| ATOM | 8440 | NZ | LYS | 4265 | 23.341 | −14.922 | 33.455 | 1.00 | 43.82 |
| ATOM | 8441 | C | LYS | 4265 | 22.250 | −12.790 | 27.471 | 1.00 | 41.02 |
| ATOM | 8442 | O | LYS | 4265 | 21.153 | −12.226 | 27.391 | 1.00 | 41.34 |
| ATOM | 8443 | N | THR | 4266 | 22.756 | −13.539 | 26.501 | 1.00 | 41.78 |
| ATOM | 8444 | CA | THR | 4266 | 22.013 | −13.804 | 25.278 | 1.00 | 42.42 |
| ATOM | 8445 | CB | THR | 4266 | 22.768 | −13.284 | 24.031 | 1.00 | 43.65 |
| ATOM | 8446 | OG1 | THR | 4266 | 22.964 | −11.867 | 24.155 | 1.00 | 45.12 |
| ATOM | 8447 | CG2 | THR | 4266 | 21.976 | −13.573 | 22.748 | 1.00 | 43.11 |
| ATOM | 8448 | C | THR | 4266 | 21.885 | −15.327 | 25.252 | 1.00 | 42.40 |
| ATOM | 8449 | O | THR | 4266 | 22.893 | −16.043 | 25.187 | 1.00 | 42.93 |
| ATOM | 8450 | N | VAL | 4267 | 20.655 | −15.823 | 25.346 | 1.00 | 41.83 |
| ATOM | 8451 | CA | VAL | 4267 | 20.439 | −17.262 | 25.352 | 1.00 | 42.05 |
| ATOM | 8452 | CB | VAL | 4267 | 20.108 | −17.767 | 26.783 | 1.00 | 41.77 |
| ATOM | 8453 | CG1 | VAL | 4267 | 21.092 | −17.165 | 27.775 | 1.00 | 41.92 |
| ATOM | 8454 | CG2 | VAL | 4267 | 18.673 | −17.424 | 27.158 | 1.00 | 40.75 |
| ATOM | 8455 | C | VAL | 4267 | 19.339 | −17.718 | 24.403 | 1.00 | 42.81 |
| ATOM | 8456 | O | VAL | 4267 | 18.484 | −16.934 | 23.978 | 1.00 | 42.07 |
| ATOM | 8457 | N | ALA | 4268 | 19.375 | −19.007 | 24.082 | 1.00 | 44.08 |
| ATOM | 8458 | CA | ALA | 4268 | 18.401 | −19.616 | 23.191 | 1.00 | 45.49 |
| ATOM | 8459 | CB | ALA | 4268 | 18.947 | −20.939 | 22.669 | 1.00 | 45.80 |
| ATOM | 8460 | C | ALA | 4268 | 17.103 | −19.852 | 23.944 | 1.00 | 45.91 |
| ATOM | 8461 | O | ALA | 4268 | 17.119 | −20.106 | 25.146 | 1.00 | 45.97 |
| ATOM | 8462 | N | LEU | 4269 | 15.981 | −19.770 | 23.238 | 1.00 | 46.85 |
| ATOM | 8463 | CA | LEU | 4269 | 14.685 | −19.991 | 23.867 | 1.00 | 47.72 |
| ATOM | 8464 | CB | LEU | 4269 | 13.576 | −20.014 | 22.815 | 1.00 | 48.40 |
| ATOM | 8465 | CG | LEU | 4269 | 12.210 | −19.430 | 23.188 | 1.00 | 48.24 |
| ATOM | 8466 | CD1 | LEU | 4269 | 11.277 | −19.590 | 21.995 | 1.00 | 48.55 |
| ATOM | 8467 | CD2 | LEU | 4269 | 11.638 | −20.118 | 24.413 | 1.00 | 48.44 |
| ATOM | 8468 | C | LEU | 4269 | 14.714 | −21.332 | 24.587 | 1.00 | 48.41 |
| ATOM | 8469 | O | LEU | 4269 | 15.317 | −22.298 | 24.105 | 1.00 | 48.84 |
| ATOM | 8470 | N | GLY | 4270 | 14.075 | −21.387 | 25.748 | 1.00 | 48.96 |
| ATOM | 8471 | CA | GLY | 4270 | 14.032 | −22.626 | 26.502 | 1.00 | 49.19 |
| ATOM | 8472 | C | GLY | 4270 | 15.258 | −22.903 | 27.351 | 1.00 | 49.25 |
| ATOM | 8473 | O | GLY | 4270 | 15.310 | −23.918 | 28.047 | 1.00 | 49.68 |
| ATOM | 8474 | N | SER | 4271 | 16.249 | −22.018 | 27.299 | 1.00 | 48.97 |
| ATOM | 8475 | CA | SER | 4271 | 17.457 | −22.211 | 28.096 | 1.00 | 49.34 |
| ATOM | 8476 | CB | SER | 4271 | 18.594 | −21.299 | 27.610 | 1.00 | 49.53 |
| ATOM | 8477 | OG | SER | 4271 | 19.096 | −21.685 | 26.345 | 1.00 | 50.54 |
| ATOM | 8478 | C | SER | 4271 | 17.202 | −21.894 | 29.569 | 1.00 | 49.44 |
| ATOM | 8479 | O | SER | 4271 | 16.155 | −21.343 | 29.935 | 1.00 | 49.11 |
| ATOM | 8480 | N | ASN | 4272 | 18.173 | −22.260 | 30.405 | 1.00 | 49.10 |
| ATOM | 8481 | CA | ASN | 4272 | 18.131 | −21.982 | 31.839 | 1.00 | 48.68 |
| ATOM | 8482 | CB | ASN | 4272 | 18.532 | −23.203 | 32.673 | 1.00 | 48.99 |
| ATOM | 8483 | CG | ASN | 4272 | 17.538 | −24.333 | 32.582 | 1.00 | 49.46 |
| ATOM | 8484 | OD1 | ASN | 4272 | 16.329 | −24.125 | 32.685 | 1.00 | 49.99 |
| ATOM | 8485 | ND2 | ASN | 4272 | 18.043 | −25.547 | 32.409 | 1.00 | 49.98 |
| ATOM | 8486 | C | ASN | 4272 | 19.199 | −20.920 | 32.040 | 1.00 | 48.17 |
| ATOM | 8487 | O | ASN | 4272 | 20.304 | −21.047 | 31.507 | 1.00 | 48.28 |
| ATOM | 8488 | N | VAL | 4273 | 18.891 | −19.875 | 32.796 | 1.00 | 47.43 |
| ATOM | 8489 | CA | VAL | 4273 | 19.882 | −18.837 | 33.016 | 1.00 | 47.20 |
| ATOM | 8490 | CB | VAL | 4273 | 19.639 | −17.632 | 32.088 | 1.00 | 47.78 |
| ATOM | 8491 | CG1 | VAL | 4273 | 18.180 | −17.228 | 32.140 | 1.00 | 49.04 |
| ATOM | 8492 | CG2 | VAL | 4273 | 20.536 | −16.466 | 32.496 | 1.00 | 49.38 |
| ATOM | 8493 | C | VAL | 4273 | 19.897 | −18.382 | 34.462 | 1.00 | 46.68 |
| ATOM | 8494 | O | VAL | 4273 | 18.872 | −18.399 | 35.147 | 1.00 | 46.75 |
| ATOM | 8495 | N | GLU | 4274 | 21.077 | −17.993 | 34.927 | 1.00 | 46.32 |
| ATOM | 8496 | CA | GLU | 4274 | 21.237 | −17.539 | 36.297 | 1.00 | 46.11 |
| ATOM | 8497 | CB | GLU | 4274 | 21.977 | −18.595 | 37.115 | 1.00 | 48.24 |
| ATOM | 8498 | CG | GLU | 4274 | 23.366 | −18.915 | 36.587 | 1.00 | 51.52 |
| ATOM | 8499 | CD | GLU | 4274 | 24.124 | −19.858 | 37.512 | 1.00 | 54.50 |
| ATOM | 8500 | OE1 | GLU | 4274 | 23.563 | −20.933 | 37.854 | 1.00 | 55.30 |
| ATOM | 8501 | OE2 | GLU | 4274 | 25.274 | −19.519 | 37.895 | 1.00 | 55.38 |
| ATOM | 8502 | C | GLU | 4274 | 22.009 | −16.232 | 36.370 | 1.00 | 44.59 |
| ATOM | 8503 | O | GLU | 4274 | 23.146 | −16.139 | 35.911 | 1.00 | 43.75 |
| ATOM | 8504 | N | PHE | 4275 | 21.378 | −15.221 | 36.949 | 1.00 | 43.08 |
| ATOM | 8505 | CA | PHE | 4275 | 22.018 | −13.931 | 37.104 | 1.00 | 41.79 |
| ATOM | 8506 | CB | PHE | 4275 | 20.983 | −12.818 | 36.970 | 1.00 | 41.26 |
| ATOM | 8507 | CG | PHE | 4275 | 20.591 | −12.519 | 35.551 | 1.00 | 39.42 |
| ATOM | 8508 | CD1 | PHE | 4275 | 21.511 | −11.965 | 34.666 | 1.00 | 39.12 |
| ATOM | 8509 | CD2 | PHE | 4275 | 19.301 | −12.774 | 35.102 | 1.00 | 38.78 |
| ATOM | 8510 | CE1 | PHE | 4275 | 21.149 | −11.667 | 33.350 | 1.00 | 39.31 |
| ATOM | 8511 | CE2 | PHE | 4275 | 18.930 | −12.480 | 33.791 | 1.00 | 38.50 |
| ATOM | 8512 | CZ | PHE | 4275 | 19.855 | −11.926 | 32.911 | 1.00 | 38.65 |
| ATOM | 8513 | C | PHE | 4275 | 22.642 | −13.916 | 38.487 | 1.00 | 41.74 |
| ATOM | 8514 | O | PHE | 4275 | 22.082 | −14.467 | 39.434 | 1.00 | 41.78 |
| ATOM | 8515 | N | MSE | 4276 | 23.808 | −13.301 | 38.603 | 1.00 | 42.11 |
| ATOM | 8516 | CA | MSE | 4276 | 24.483 | −13.243 | 39.883 | 1.00 | 43.17 |
| ATOM | 8517 | CB | MSE | 4276 | 25.859 | −13.897 | 39.791 | 1.00 | 45.71 |
| ATOM | 8518 | CG | MSE | 4276 | 25.837 | −15.411 | 39.658 | 1.00 | 49.58 |
| ATOM | 8519 | SE | MSE | 4276 | 27.523 | −15.985 | 39.385 | 1.00 | 54.60 |
| ATOM | 8520 | CE | MSE | 4276 | 28.273 | −15.667 | 41.037 | 1.00 | 52.61 |
| ATOM | 8521 | C | MSE | 4276 | 24.650 | −11.821 | 40.382 | 1.00 | 42.83 |
| ATOM | 8522 | O | MSE | 4276 | 24.603 | −10.857 | 39.609 | 1.00 | 41.81 |
| ATOM | 8523 | N | CYS | 4277 | 24.858 | −11.704 | 41.689 | 1.00 | 42.60 |
| ATOM | 8524 | CA | CYS | 4277 | 25.058 | −10.413 | 42.323 | 1.00 | 42.37 |
| ATOM | 8525 | CB | CYS | 4277 | 23.721 | −9.753 | 42.616 | 1.00 | 43.78 |
| ATOM | 8526 | SG | CYS | 4277 | 23.968 | −8.039 | 43.081 | 1.00 | 49.21 |
| ATOM | 8527 | C | CYS | 4277 | 25.841 | −10.539 | 43.623 | 1.00 | 41.33 |
| ATOM | 8528 | O | CYS | 4277 | 25.383 | −11.173 | 44.578 | 1.00 | 41.69 |
| ATOM | 8529 | N | LYS | 4278 | 27.021 | −9.930 | 43.654 | 1.00 | 40.30 |
| ATOM | 8530 | CA | LYS | 4278 | 27.875 | −9.964 | 44.838 | 1.00 | 39.00 |
| ATOM | 8531 | CB | LYS | 4278 | 29.335 | −10.196 | 44.421 | 1.00 | 41.27 |
| ATOM | 8532 | CG | LYS | 4278 | 29.601 | −11.645 | 43.985 | 1.00 | 44.09 |
| ATOM | 8533 | CD | LYS | 4278 | 31.030 | −11.859 | 43.488 | 1.00 | 45.93 |
| ATOM | 8534 | CE | LYS | 4278 | 31.209 | −13.304 | 42.984 | 1.00 | 47.72 |
| ATOM | 8535 | NZ | LYS | 4278 | 32.561 | −13.574 | 42.385 | 1.00 | 48.32 |
| ATOM | 8536 | C | LYS | 4278 | 27.733 | −8.669 | 45.629 | 1.00 | 36.58 |
| ATOM | 8537 | O | LYS | 4278 | 28.093 | −7.596 | 45.146 | 1.00 | 36.57 |
| ATOM | 8538 | N | VAL | 4279 | 27.213 | −8.781 | 46.848 | 1.00 | 33.81 |
| ATOM | 8539 | CA | VAL | 4279 | 26.992 | −7.616 | 47.688 | 1.00 | 31.09 |
| ATOM | 8540 | CB | VAL | 4279 | 25.530 | −7.588 | 48.202 | 1.00 | 30.28 |
| ATOM | 8541 | CG1 | VAL | 4279 | 25.299 | −6.379 | 49.095 | 1.00 | 29.47 |
| ATOM | 8542 | CG2 | VAL | 4279 | 24.578 | −7.574 | 47.036 | 1.00 | 28.79 |
| ATOM | 8543 | C | VAL | 4279 | 27.903 | −7.541 | 48.899 | 1.00 | 30.56 |
| ATOM | 8544 | O | VAL | 4279 | 28.202 | −8.553 | 49.531 | 1.00 | 30.61 |
| ATOM | 8545 | N | TYR | 4280 | 28.340 | −6.328 | 49.218 | 1.00 | 29.91 |
| ATOM | 8546 | CA | TYR | 4280 | 29.154 | −6.096 | 50.394 | 1.00 | 28.61 |
| ATOM | 8547 | CB | TYR | 4280 | 30.548 | −5.617 | 50.025 | 1.00 | 29.29 |
| ATOM | 8548 | CG | TYR | 4280 | 31.369 | −5.268 | 51.241 | 1.00 | 31.38 |
| ATOM | 8549 | CD1 | TYR | 4280 | 32.037 | −6.248 | 51.963 | 1.00 | 31.74 |
| ATOM | 8550 | CE1 | TYR | 4280 | 32.751 | −5.930 | 53.116 | 1.00 | 33.64 |
| ATOM | 8551 | CD2 | TYR | 4280 | 31.435 | −3.952 | 51.701 | 1.00 | 32.92 |
| ATOM | 8552 | CE2 | TYR | 4280 | 32.144 | −3.620 | 52.848 | 1.00 | 34.12 |
| ATOM | 8553 | CZ | TYR | 4280 | 32.803 | −4.608 | 53.555 | 1.00 | 34.51 |
| ATOM | 8554 | OH | TYR | 4280 | 33.525 | −4.269 | 54.686 | 1.00 | 34.10 |
| ATOM | 8555 | C | TYR | 4280 | 28.451 | −5.028 | 51.221 | 1.00 | 28.08 |
| ATOM | 8556 | O | TYR | 4280 | 28.012 | −4.006 | 50.695 | 1.00 | 27.49 |
| ATOM | 8557 | N | SER | 4281 | 28.340 | −5.272 | 52.519 | 1.00 | 27.77 |
| ATOM | 8558 | CA | SER | 4281 | 27.684 | −4.326 | 53.406 | 1.00 | 27.31 |
| ATOM | 8559 | CB | SER | 4281 | 26.175 | −4.359 | 53.156 | 1.00 | 27.11 |
| ATOM | 8560 | OG | SER | 4281 | 25.485 | −3.450 | 53.983 | 1.00 | 26.16 |
| ATOM | 8561 | C | SER | 4281 | 27.976 | −4.697 | 54.851 | 1.00 | 27.48 |
| ATOM | 8562 | O | SER | 4281 | 27.828 | −5.856 | 55.230 | 1.00 | 27.58 |
| ATOM | 8563 | N | ASP | 4282 | 28.404 | −3.719 | 55.649 | 1.00 | 28.41 |
| ATOM | 8564 | CA | ASP | 4282 | 28.701 | −3.955 | 57.067 | 1.00 | 28.78 |
| ATOM | 8565 | CB | ASP | 4282 | 29.438 | −2.757 | 57.676 | 1.00 | 29.98 |
| ATOM | 8566 | CG | ASP | 4282 | 29.908 | −3.015 | 59.085 | 1.00 | 31.16 |
| ATOM | 8567 | OD1 | ASP | 4282 | 30.629 | −2.158 | 59.629 | 1.00 | 32.30 |
| ATOM | 8568 | OD2 | ASP | 4282 | 29.565 | −4.069 | 59.658 | 1.00 | 33.85 |
| ATOM | 8569 | C | ASP | 4282 | 27.349 | −4.172 | 57.726 | 1.00 | 28.36 |
| ATOM | 8570 | O | ASP | 4282 | 27.072 | −5.250 | 58.235 | 1.00 | 28.55 |
| ATOM | 8571 | N | PRO | 4283 | 26.481 | −3.153 | 57.725 | 1.00 | 28.52 |
| ATOM | 8572 | CD | PRO | 4283 | 26.579 | −1.736 | 57.330 | 1.00 | 27.31 |
| ATOM | 8573 | CA | PRO | 4283 | 25.188 | −3.433 | 58.358 | 1.00 | 28.80 |
| ATOM | 8574 | CB | PRO | 4283 | 24.484 | −2.085 | 58.331 | 1.00 | 28.18 |
| ATOM | 8575 | CG | PRO | 4283 | 25.640 | −1.097 | 58.292 | 1.00 | 28.47 |
| ATOM | 8576 | C | PRO | 4283 | 24.500 | −4.449 | 57.445 | 1.00 | 29.41 |
| ATOM | 8577 | O | PRO | 4283 | 24.733 | −4.456 | 56.228 | 1.00 | 28.92 |
| ATOM | 8578 | N | GLN | 4284 | 23.667 | −5.301 | 58.029 | 1.00 | 29.87 |
| ATOM | 8579 | CA | GLN | 4284 | 22.949 | −6.327 | 57.283 | 1.00 | 30.26 |
| ATOM | 8580 | CB | GLN | 4284 | 21.990 | −7.037 | 58.229 | 1.00 | 31.50 |
| ATOM | 8581 | CG | GLN | 4284 | 21.834 | −8.513 | 57.951 | 1.00 | 32.38 |
| ATOM | 8582 | CD | GLN | 4284 | 23.165 | −9.224 | 57.909 | 1.00 | 33.02 |
| ATOM | 8583 | OE1 | GLN | 4284 | 23.909 | −9.246 | 58.895 | 1.00 | 33.01 |
| ATOM | 8584 | NE2 | GLN | 4284 | 23.478 | −9.811 | 56.761 | 1.00 | 33.12 |
| ATOM | 8585 | C | GLN | 4284 | 22.182 | −5.703 | 56.109 | 1.00 | 30.48 |
| ATOM | 8586 | O | GLN | 4284 | 21.371 | −4.799 | 56.300 | 1.00 | 30.46 |
| ATOM | 8587 | N | PRO | 4285 | 22.457 | −6.162 | 54.870 | 1.00 | 30.32 |
| ATOM | 8588 | CD | PRO | 4285 | 23.680 | −6.866 | 54.452 | 1.00 | 30.16 |
| ATOM | 8589 | CA | PRO | 4285 | 21.752 | −5.605 | 53.709 | 1.00 | 30.02 |
| ATOM | 8590 | CB | PRO | 4285 | 22.827 | −5.622 | 52.632 | 1.00 | 30.18 |
| ATOM | 8591 | CG | PRO | 4285 | 23.552 | −6.893 | 52.939 | 1.00 | 30.10 |
| ATOM | 8592 | C | PRO | 4285 | 20.522 | −6.394 | 53.289 | 1.00 | 30.48 |
| ATOM | 8593 | O | PRO | 4285 | 20.418 | −7.600 | 53.517 | 1.00 | 30.39 |
| ATOM | 8594 | N | HIS | 4286 | 19.579 | −5.700 | 52.670 | 1.00 | 30.89 |
| ATOM | 8595 | CA | HIS | 4286 | 18.371 | −6.334 | 52.210 | 1.00 | 31.17 |
| ATOM | 8596 | CB | HIS | 4286 | 17.156 | −5.569 | 52.705 | 1.00 | 32.91 |
| ATOM | 8597 | CG | HIS | 4286 | 15.865 | −6.195 | 52.299 | 1.00 | 34.87 |
| ATOM | 8598 | CD2 | HIS | 4286 | 15.260 | −7.333 | 52.714 | 1.00 | 35.51 |
| ATOM | 8599 | ND1 | HIS | 4286 | 15.093 | −5.703 | 51.268 | 1.00 | 36.13 |
| ATOM | 8600 | CE1 | HIS | 4286 | 14.070 | −6.516 | 51.063 | 1.00 | 35.97 |
| ATOM | 8601 | NE2 | HIS | 4286 | 14.148 | −7.512 | 51.928 | 1.00 | 35.58 |
| ATOM | 8602 | C | HIS | 4286 | 18.406 | −6.344 | 50.694 | 1.00 | 31.58 |
| ATOM | 8603 | O | HIS | 4286 | 18.322 | −5.290 | 50.063 | 1.00 | 31.81 |
| ATOM | 8604 | N | ILE | 4287 | 18.532 | −7.538 | 50.117 | 1.00 | 31.34 |
| ATOM | 8605 | CA | ILE | 4287 | 18.610 | −7.702 | 48.675 | 1.00 | 31.28 |
| ATOM | 8606 | CB | ILE | 4287 | 19.636 | −8.805 | 48.328 | 1.00 | 32.06 |
| ATOM | 8607 | CG2 | ILE | 4287 | 19.668 | −9.081 | 46.816 | 1.00 | 31.41 |
| ATOM | 8608 | CG1 | ILE | 4287 | 21.016 | −8.370 | 48.828 | 1.00 | 32.58 |
| ATOM | 8609 | CD1 | ILE | 4287 | 22.124 | −9.349 | 48.512 | 1.00 | 33.57 |
| ATOM | 8610 | C | ILE | 4287 | 17.252 | −8.015 | 48.068 | 1.00 | 31.79 |
| ATOM | 8611 | O | ILE | 4287 | 16.385 | −8.584 | 48.727 | 1.00 | 31.80 |
| ATOM | 8612 | N | GLN | 4288 | 17.082 | −7.652 | 46.801 | 1.00 | 32.04 |
| ATOM | 8613 | CA | GLN | 4288 | 15.815 | −7.841 | 46.103 | 1.00 | 33.12 |
| ATOM | 8614 | CB | GLN | 4288 | 14.944 | −6.615 | 46.388 | 1.00 | 33.99 |
| ATOM | 8615 | CG | GLN | 4288 | 13.443 | −6.784 | 46.322 | 1.00 | 37.24 |
| ATOM | 8616 | CD | GLN | 4288 | 12.696 | −5.542 | 46.856 | 1.00 | 38.86 |
| ATOM | 8617 | OE1 | GLN | 4288 | 11.470 | −5.544 | 46.982 | 1.00 | 39.88 |
| ATOM | 8618 | NE2 | GLN | 4288 | 13.443 | −4.484 | 47.170 | 1.00 | 38.58 |
| ATOM | 8619 | C | GLN | 4288 | 16.103 | −7.929 | 44.607 | 1.00 | 33.15 |
| ATOM | 8620 | O | GLN | 4288 | 16.927 | −7.179 | 44.098 | 1.00 | 33.78 |
| ATOM | 8621 | N | TRP | 4289 | 15.450 | −8.852 | 43.909 | 1.00 | 33.18 |
| ATOM | 8622 | CA | TRP | 4289 | 15.626 | −8.978 | 42.459 | 1.00 | 33.22 |
| ATOM | 8623 | CB | TRP | 4289 | 15.904 | −10.424 | 42.045 | 1.00 | 33.61 |
| ATOM | 8624 | CG | TRP | 4289 | 17.289 | −10.884 | 42.305 | 1.00 | 34.06 |
| ATOM | 8625 | CD2 | TRP | 4289 | 18.408 | −10.752 | 41.428 | 1.00 | 34.66 |
| ATOM | 8626 | CE2 | TRP | 4289 | 19.520 | −11.344 | 42.076 | 1.00 | 34.84 |
| ATOM | 8627 | CE3 | TRP | 4289 | 18.581 | −10.190 | 40.157 | 1.00 | 34.70 |
| ATOM | 8628 | CD1 | TRP | 4289 | 17.750 | −11.528 | 43.423 | 1.00 | 34.15 |
| ATOM | 8629 | NE1 | TRP | 4289 | 19.088 | −11.809 | 43.291 | 1.00 | 34.64 |
| ATOM | 8630 | CZ2 | TRP | 4289 | 20.791 | −11.391 | 41.496 | 1.00 | 35.28 |
| ATOM | 8631 | CZ3 | TRP | 4289 | 19.845 | −10.237 | 39.574 | 1.00 | 35.73 |
| ATOM | 8632 | CH2 | TRP | 4289 | 20.936 | −10.835 | 40.247 | 1.00 | 36.23 |
| ATOM | 8633 | C | TRP | 4289 | 14.358 | −8.500 | 41.746 | 1.00 | 33.64 |
| ATOM | 8634 | O | TRP | 4289 | 13.250 | −8.918 | 42.077 | 1.00 | 32.96 |
| ATOM | 8635 | N | LEU | 4290 | 14.523 | −7.622 | 40.768 | 1.00 | 34.40 |
| ATOM | 8636 | CA | LEU | 4290 | 13.376 | −7.103 | 40.040 | 1.00 | 35.84 |
| ATOM | 8637 | CB | LEU | 4290 | 13.157 | −5.617 | 40.362 | 1.00 | 36.53 |
| ATOM | 8638 | CG | LEU | 4290 | 13.168 | −5.300 | 41.863 | 1.00 | 37.20 |
| ATOM | 8639 | CD1 | LEU | 4290 | 14.603 | −5.239 | 42.327 | 1.00 | 36.89 |
| ATOM | 8640 | CD2 | LEU | 4290 | 12.492 | −3.973 | 42.145 | 1.00 | 38.20 |
| ATOM | 8641 | C | LEU | 4290 | 13.523 | −7.283 | 38.539 | 1.00 | 36.16 |
| ATOM | 8642 | O | LEU | 4290 | 14.636 | −7.363 | 38.008 | 1.00 | 35.34 |
| ATOM | 8643 | N | LYS | 4291 | 12.379 | −7.376 | 37.872 | 1.00 | 37.15 |
| ATOM | 8644 | CA | LYS | 4291 | 12.323 | −7.514 | 36.423 | 1.00 | 38.48 |
| ATOM | 8645 | CB | LYS | 4291 | 11.609 | −8.817 | 36.026 | 1.00 | 39.76 |
| ATOM | 8646 | CG | LYS | 4291 | 11.108 | −8.901 | 34.564 | 1.00 | 40.60 |
| ATOM | 8647 | CD | LYS | 4291 | 12.246 | −8.830 | 33.535 | 1.00 | 42.97 |
| ATOM | 8648 | CE | LYS | 4291 | 11.820 | −9.328 | 32.138 | 1.00 | 44.27 |
| ATOM | 8649 | NZ | LYS | 4291 | 10.669 | −8.590 | 31.524 | 1.00 | 44.72 |
| ATOM | 8650 | C | LYS | 4291 | 11.541 | −6.306 | 35.924 | 1.00 | 38.86 |
| ATOM | 8651 | O | LYS | 4291 | 10.430 | −6.043 | 36.390 | 1.00 | 37.83 |
| ATOM | 8652 | N | HIS | 4292 | 12.142 | −5.556 | 35.007 | 1.00 | 40.21 |
| ATOM | 8653 | CA | HIS | 4292 | 11.490 | −4.384 | 34.433 | 1.00 | 42.41 |
| ATOM | 8654 | CB | HIS | 4292 | 12.525 | −3.516 | 33.725 | 1.00 | 41.96 |
| ATOM | 8655 | CG | HIS | 4292 | 13.440 | −2.809 | 34.668 | 1.00 | 41.96 |
| ATOM | 8656 | CD2 | HIS | 4292 | 14.624 | −3.182 | 35.207 | 1.00 | 42.64 |
| ATOM | 8657 | ND1 | HIS | 4292 | 13.104 | −1.617 | 35.267 | 1.00 | 41.77 |
| ATOM | 8658 | CE1 | HIS | 4292 | 14.038 | −1.288 | 36.141 | 1.00 | 42.75 |
| ATOM | 8659 | NE2 | HIS | 4292 | 14.971 | −2.221 | 36.126 | 1.00 | 42.35 |
| ATOM | 8660 | C | HIS | 4292 | 10.395 | −4.826 | 33.471 | 1.00 | 44.25 |
| ATOM | 8661 | O | HIS | 4292 | 10.652 | −5.556 | 32.507 | 1.00 | 43.41 |
| ATOM | 8662 | N | ILE | 4293 | 9.180 | −4.366 | 33.761 | 1.00 | 47.48 |
| ATOM | 8663 | CA | ILE | 4293 | 7.979 | −4.708 | 33.009 | 1.00 | 51.04 |
| ATOM | 8664 | CB | ILE | 4293 | 6.918 | −5.343 | 33.965 | 1.00 | 51.20 |
| ATOM | 8665 | CG2 | ILE | 4293 | 5.679 | −5.771 | 33.198 | 1.00 | 51.79 |
| ATOM | 8666 | CG1 | ILE | 4293 | 7.509 | −6.568 | 34.652 | 1.00 | 51.93 |
| ATOM | 8667 | CD1 | ILE | 4293 | 8.030 | −7.621 | 33.680 | 1.00 | 52.28 |
| ATOM | 8668 | C | ILE | 4293 | 7.321 | −3.524 | 32.296 | 1.00 | 53.15 |
| ATOM | 8669 | O | ILE | 4293 | 7.620 | −2.354 | 32.574 | 1.00 | 53.09 |
| ATOM | 8670 | N | GLU | 4294 | 6.416 | −3.857 | 31.376 | 1.00 | 55.65 |
| ATOM | 8671 | CA | GLU | 4294 | 5.657 | −2.870 | 30.618 | 1.00 | 58.14 |
| ATOM | 8672 | CB | GLU | 4294 | 5.801 | −3.117 | 29.112 | 1.00 | 57.19 |
| ATOM | 8673 | C | GLU | 4294 | 4.183 | −2.960 | 31.012 | 1.00 | 59.77 |
| ATOM | 8674 | O | GLU | 4294 | 3.759 | −3.897 | 31.689 | 1.00 | 59.44 |
| ATOM | 8675 | N | VAL | 4295 | 3.413 | −1.967 | 30.593 | 1.00 | 62.69 |
| ATOM | 8676 | CA | VAL | 4295 | 1.981 | −1.915 | 30.868 | 1.00 | 65.41 |
| ATOM | 8677 | CB | VAL | 4295 | 1.700 | −1.091 | 32.152 | 1.00 | 65.44 |
| ATOM | 8678 | CG1 | VAL | 4295 | 0.211 | −0.817 | 32.307 | 1.00 | 65.73 |
| ATOM | 8679 | CG2 | VAL | 4295 | 2.211 | −1.861 | 33.366 | 1.00 | 64.96 |
| ATOM | 8680 | C | VAL | 4295 | 1.323 | −1.287 | 29.636 | 1.00 | 67.29 |
| ATOM | 8681 | O | VAL | 4295 | 0.118 | −1.025 | 29.611 | 1.00 | 67.51 |
| ATOM | 8682 | N | ASN | 4296 | 2.154 | −1.083 | 28.612 | 1.00 | 69.59 |
| ATOM | 8683 | CA | ASN | 4296 | 1.786 | −0.515 | 27.311 | 1.00 | 71.69 |
| ATOM | 8684 | CB | ASN | 4296 | 1.279 | 0.925 | 27.459 | 1.00 | 72.56 |
| ATOM | 8685 | CG | ASN | 4296 | −0.044 | 1.004 | 28.180 | 1.00 | 73.76 |
| ATOM | 8686 | OD1 | ASN | 4296 | −1.025 | 0.378 | 27.767 | 1.00 | 74.52 |
| ATOM | 8687 | ND2 | ASN | 4296 | −0.082 | 1.766 | 29.273 | 1.00 | 74.09 |
| ATOM | 8688 | C | ASN | 4296 | 3.067 | −0.498 | 26.482 | 1.00 | 72.69 |
| ATOM | 8689 | O | ASN | 4296 | 3.666 | −1.540 | 26.200 | 1.00 | 73.05 |
| ATOM | 8690 | N | GLY | 4297 | 3.467 | 0.705 | 26.089 | 1.00 | 73.53 |
| ATOM | 8691 | CA | GLY | 4297 | 4.698 | 0.898 | 25.349 | 1.00 | 74.28 |
| ATOM | 8692 | C | GLY | 4297 | 5.497 | 1.723 | 26.338 | 1.00 | 74.92 |
| ATOM | 8693 | O | GLY | 4297 | 6.571 | 2.267 | 26.039 | 1.00 | 74.91 |
| ATOM | 8694 | N | SER | 4298 | 4.924 | 1.806 | 27.540 | 1.00 | 74.92 |
| ATOM | 8695 | CA | SER | 4298 | 5.496 | 2.548 | 28.652 | 1.00 | 74.63 |
| ATOM | 8696 | CB | SER | 4298 | 4.408 | 3.393 | 29.335 | 1.00 | 74.57 |
| ATOM | 8697 | OG | SER | 4298 | 3.226 | 2.642 | 29.564 | 1.00 | 75.04 |
| ATOM | 8698 | C | SER | 4298 | 6.164 | 1.624 | 29.667 | 1.00 | 74.22 |
| ATOM | 8699 | O | SER | 4298 | 5.498 | 0.876 | 30.388 | 1.00 | 73.80 |
| ATOM | 8700 | N | LYS | 4299 | 7.493 | 1.676 | 29.689 | 1.00 | 73.81 |
| ATOM | 8701 | CA | LYS | 4299 | 8.285 | 0.884 | 30.611 | 1.00 | 73.37 |
| ATOM | 8702 | CB | LYS | 4299 | 9.704 | 0.700 | 30.070 | 1.00 | 73.41 |
| ATOM | 8703 | CG | LYS | 4299 | 9.779 | −0.112 | 28.783 | 1.00 | 73.66 |
| ATOM | 8704 | CD | LYS | 4299 | 11.219 | −0.283 | 28.314 | 1.00 | 73.93 |
| ATOM | 8705 | CE | LYS | 4299 | 11.301 | −1.171 | 27.074 | 1.00 | 74.43 |
| ATOM | 8706 | NZ | LYS | 4299 | 12.705 | −1.372 | 26.586 | 1.00 | 74.54 |
| ATOM | 8707 | C | LYS | 4299 | 8.318 | 1.655 | 31.920 | 1.00 | 72.94 |
| ATOM | 8708 | O | LYS | 4299 | 8.678 | 1.119 | 32.969 | 1.00 | 73.16 |
| ATOM | 8709 | N | ILE | 4300 | 7.932 | 2.924 | 31.841 | 1.00 | 72.42 |
| ATOM | 8710 | CA | ILE | 4300 | 7.893 | 3.798 | 33.003 | 1.00 | 72.36 |
| ATOM | 8711 | CB | ILE | 4300 | 8.580 | 5.153 | 32.717 | 1.00 | 72.06 |
| ATOM | 8712 | CG2 | ILE | 4300 | 8.693 | 5.967 | 34.002 | 1.00 | 71.81 |
| ATOM | 8713 | CG1 | ILE | 4300 | 9.974 | 4.920 | 32.136 | 1.00 | 71.69 |
| ATOM | 8714 | CD1 | ILE | 4300 | 10.901 | 4.157 | 33.055 | 1.00 | 72.04 |
| ATOM | 8715 | C | ILE | 4300 | 6.434 | 4.050 | 33.359 | 1.00 | 72.49 |
| ATOM | 8716 | O | ILE | 4300 | 5.548 | 3.904 | 32.517 | 1.00 | 72.30 |
| ATOM | 8717 | N | GLY | 4301 | 6.193 | 4.424 | 34.609 | 1.00 | 72.80 |
| ATOM | 8718 | CA | GLY | 4301 | 4.838 | 4.680 | 35.057 | 1.00 | 73.35 |
| ATOM | 8719 | C | GLY | 4301 | 4.440 | 6.147 | 35.057 | 1.00 | 73.57 |
| ATOM | 8720 | O | GLY | 4301 | 5.252 | 7.022 | 34.731 | 1.00 | 73.30 |
| ATOM | 8721 | N | PRO | 4302 | 3.179 | 6.444 | 35.426 | 1.00 | 73.73 |
| ATOM | 8722 | CD | PRO | 4302 | 2.148 | 5.443 | 35.742 | 1.00 | 73.72 |
| ATOM | 8723 | CA | PRO | 4302 | 2.613 | 7.801 | 35.485 | 1.00 | 73.40 |
| ATOM | 8724 | CB | PRO | 4302 | 1.111 | 7.550 | 35.656 | 1.00 | 73.95 |
| ATOM | 8725 | CG | PRO | 4302 | 0.918 | 6.109 | 35.202 | 1.00 | 74.03 |
| ATOM | 8726 | C | PRO | 4302 | 3.189 | 8.579 | 36.659 | 1.00 | 72.79 |
| ATOM | 8727 | O | PRO | 4302 | 2.988 | 9.791 | 36.781 | 1.00 | 72.49 |
| ATOM | 8728 | N | ASP | 4303 | 3.901 | 7.856 | 37.520 | 1.00 | 72.01 |
| ATOM | 8729 | CA | ASP | 4303 | 4.529 | 8.429 | 38.701 | 1.00 | 71.14 |
| ATOM | 8730 | CB | ASP | 4303 | 4.234 | 7.553 | 39.925 | 1.00 | 71.50 |
| ATOM | 8731 | CG | ASP | 4303 | 4.633 | 6.100 | 39.715 | 1.00 | 71.75 |
| ATOM | 8732 | OD1 | ASP | 4303 | 4.619 | 5.333 | 40.701 | 1.00 | 71.77 |
| ATOM | 8733 | OD2 | ASP | 4303 | 4.957 | 5.724 | 38.566 | 1.00 | 72.03 |
| ATOM | 8734 | C | ASP | 4303 | 6.043 | 8.566 | 38.510 | 1.00 | 70.24 |
| ATOM | 8735 | O | ASP | 4303 | 6.775 | 8.824 | 39.470 | 1.00 | 70.13 |
| ATOM | 8736 | N | ASN | 4304 | 6.501 | 8.383 | 37.271 | 1.00 | 68.93 |
| ATOM | 8737 | CA | ASN | 4304 | 7.924 | 8.503 | 36.928 | 1.00 | 67.67 |
| ATOM | 8738 | CB | ASN | 4304 | 8.505 | 9.789 | 37.552 | 1.00 | 67.88 |
| ATOM | 8739 | CG | ASN | 4304 | 9.905 | 10.121 | 37.047 | 1.00 | 68.24 |
| ATOM | 8740 | OD1 | ASN | 4304 | 10.157 | 10.142 | 35.835 | 1.00 | 67.71 |
| ATOM | 8741 | ND2 | ASN | 4304 | 10.820 | 10.403 | 37.979 | 1.00 | 67.87 |
| ATOM | 8742 | C | ASN | 4304 | 8.744 | 7.277 | 37.351 | 1.00 | 66.27 |
| ATOM | 8743 | O | ASN | 4304 | 9.934 | 7.177 | 37.044 | 1.00 | 66.12 |
| ATOM | 8744 | N | LEU | 4305 | 8.106 | 6.352 | 38.059 | 1.00 | 64.57 |
| ATOM | 8745 | CA | LEU | 4305 | 8.776 | 5.131 | 38.490 | 1.00 | 62.93 |
| ATOM | 8746 | CB | LEU | 4305 | 8.261 | 4.681 | 39.858 | 1.00 | 62.73 |
| ATOM | 8747 | CG | LEU | 4305 | 8.769 | 5.458 | 41.074 | 1.00 | 62.83 |
| ATOM | 8748 | CD1 | LEU | 4305 | 10.289 | 5.484 | 41.045 | 1.00 | 62.06 |
| ATOM | 8749 | CD2 | LEU | 4305 | 8.215 | 6.870 | 41.071 | 1.00 | 63.05 |
| ATOM | 8750 | C | LEU | 4305 | 8.517 | 4.039 | 37.456 | 1.00 | 61.84 |
| ATOM | 8751 | O | LEU | 4305 | 7.414 | 3.932 | 36.921 | 1.00 | 62.08 |
| ATOM | 8752 | N | PRO | 4306 | 9.536 | 3.216 | 37.156 | 1.00 | 60.36 |
| ATOM | 8753 | CD | PRO | 4306 | 10.923 | 3.306 | 37.648 | 1.00 | 59.81 |
| ATOM | 8754 | CA | PRO | 4306 | 9.395 | 2.134 | 36.176 | 1.00 | 59.10 |
| ATOM | 8755 | CB | PRO | 4306 | 10.842 | 1.805 | 35.832 | 1.00 | 59.65 |
| ATOM | 8756 | CG | PRO | 4306 | 11.530 | 2.007 | 37.153 | 1.00 | 59.97 |
| ATOM | 8757 | C | PRO | 4306 | 8.637 | 0.916 | 36.712 | 1.00 | 57.89 |
| ATOM | 8758 | O | PRO | 4306 | 8.847 | 0.499 | 37.854 | 1.00 | 57.26 |
| ATOM | 8759 | N | TYR | 4307 | 7.757 | 0.354 | 35.884 | 1.00 | 56.52 |
| ATOM | 8760 | CA | TYR | 4307 | 6.986 | −0.825 | 36.278 | 1.00 | 55.10 |
| ATOM | 8761 | CB | TYR | 4307 | 6.002 | −1.248 | 35.175 | 1.00 | 57.23 |
| ATOM | 8762 | CG | TYR | 4307 | 4.952 | −0.221 | 34.785 | 1.00 | 59.34 |
| ATOM | 8763 | CD1 | TYR | 4307 | 4.897 | 0.282 | 33.483 | 1.00 | 60.44 |
| ATOM | 8764 | CE1 | TYR | 4307 | 3.918 | 1.210 | 33.099 | 1.00 | 61.23 |
| ATOM | 8765 | CD2 | TYR | 4307 | 3.998 | 0.230 | 35.704 | 1.00 | 60.43 |
| ATOM | 8766 | CE2 | TYR | 4307 | 3.009 | 1.160 | 35.328 | 1.00 | 61.18 |
| ATOM | 8767 | CZ | TYR | 4307 | 2.978 | 1.641 | 34.023 | 1.00 | 61.53 |
| ATOM | 8768 | OH | TYR | 4307 | 2.005 | 2.536 | 33.630 | 1.00 | 61.91 |
| ATOM | 8769 | C | TYR | 4307 | 7.962 | −1.974 | 36.521 | 1.00 | 52.82 |
| ATOM | 8770 | O | TYR | 4307 | 8.680 | −2.386 | 35.608 | 1.00 | 52.65 |
| ATOM | 8771 | N | VAL | 4308 | 7.995 | −2.479 | 37.750 | 1.00 | 49.77 |
| ATOM | 8772 | CA | VAL | 4308 | 8.880 | −3.588 | 38.087 | 1.00 | 47.58 |
| ATOM | 8773 | CB | VAL | 4308 | 10.029 | −3.149 | 39.033 | 1.00 | 47.43 |
| ATOM | 8774 | CG1 | VAL | 4308 | 10.777 | −1.965 | 38.421 | 1.00 | 47.27 |
| ATOM | 8775 | CG2 | VAL | 4308 | 9.481 | −2.806 | 40.406 | 1.00 | 46.48 |
| ATOM | 8776 | C | VAL | 4308 | 8.112 | −4.723 | 38.747 | 1.00 | 46.29 |
| ATOM | 8777 | O | VAL | 4308 | 7.031 | −4.525 | 39.294 | 1.00 | 45.80 |
| ATOM | 8778 | N | GLN | 4309 | 8.686 | −5.916 | 38.691 | 1.00 | 45.31 |
| ATOM | 8779 | CA | GLN | 4309 | 8.067 | −7.097 | 39.275 | 1.00 | 44.32 |
| ATOM | 8780 | CB | GLN | 4309 | 7.771 | −8.107 | 38.162 | 1.00 | 46.24 |
| ATOM | 8781 | CG | GLN | 4309 | 6.638 | −9.078 | 38.430 | 1.00 | 48.39 |
| ATOM | 8782 | CD | GLN | 4309 | 6.503 | −10.115 | 37.316 | 1.00 | 50.04 |
| ATOM | 8783 | OE1 | GLN | 4309 | 6.523 | −9.784 | 36.121 | 1.00 | 50.71 |
| ATOM | 8784 | NE2 | GLN | 4309 | 6.360 | −11.375 | 37.704 | 1.00 | 51.01 |
| ATOM | 8785 | C | GLN | 4309 | 9.074 | −7.688 | 40.252 | 1.00 | 42.69 |
| ATOM | 8786 | O | GLN | 4309 | 10.197 | −8.016 | 39.856 | 1.00 | 42.33 |
| ATOM | 8787 | N | ILE | 4310 | 8.685 | −7.804 | 41.520 | 1.00 | 40.82 |
| ATOM | 8788 | CA | ILE | 4310 | 9.562 | −8.375 | 42.540 | 1.00 | 39.61 |
| ATOM | 8789 | CB | ILE | 4310 | 8.969 | −8.252 | 43.951 | 1.00 | 39.85 |
| ATOM | 8790 | CG2 | ILE | 4310 | 10.037 | −8.605 | 44.971 | 1.00 | 39.56 |
| ATOM | 8791 | CG1 | ILE | 4310 | 8.390 | −6.853 | 44.184 | 1.00 | 40.14 |
| ATOM | 8792 | CD1 | ILE | 4310 | 9.402 | −5.748 | 44.175 | 1.00 | 40.54 |
| ATOM | 8793 | C | ILE | 4310 | 9.672 | −9.868 | 42.262 | 1.00 | 39.37 |
| ATOM | 8794 | O | ILE | 4310 | 8.682 | −10.591 | 42.365 | 1.00 | 39.52 |
| ATOM | 8795 | N | LEU | 4311 | 10.866 | −10.341 | 41.935 | 1.00 | 38.63 |
| ATOM | 8796 | CA | LEU | 4311 | 11.041 | −11.749 | 41.636 | 1.00 | 37.84 |
| ATOM | 8797 | CB | LEU | 4311 | 11.949 | −11.904 | 40.413 | 1.00 | 37.65 |
| ATOM | 8798 | CG | LEU | 4311 | 11.451 | −11.245 | 39.122 | 1.00 | 36.92 |
| ATOM | 8799 | CD1 | LEU | 4311 | 12.538 | −11.305 | 38.072 | 1.00 | 37.35 |
| ATOM | 8800 | CD2 | LEU | 4311 | 10.200 | −11.937 | 38.630 | 1.00 | 36.20 |
| ATOM | 8801 | C | LEU | 4311 | 11.606 | −12.537 | 42.808 | 1.00 | 38.39 |
| ATOM | 8802 | O | LEU | 4311 | 11.436 | −13.755 | 42.877 | 1.00 | 38.84 |
| ATOM | 8803 | N | LYS | 4312 | 12.269 | −11.849 | 43.731 | 1.00 | 38.08 |
| ATOM | 8804 | CA | LYS | 4312 | 12.863 | −12.521 | 44.878 | 1.00 | 37.58 |
| ATOM | 8805 | CB | LYS | 4312 | 14.131 | −13.231 | 44.420 | 1.00 | 37.66 |
| ATOM | 8806 | CG | LYS | 4312 | 14.555 | −14.437 | 45.236 | 1.00 | 37.49 |
| ATOM | 8807 | CD | LYS | 4312 | 15.793 | −15.026 | 44.581 | 1.00 | 37.45 |
| ATOM | 8808 | CE | LYS | 4312 | 16.243 | −16.316 | 45.226 | 1.00 | 37.77 |
| ATOM | 8809 | NZ | LYS | 4312 | 17.437 | −16.854 | 44.516 | 1.00 | 36.80 |
| ATOM | 8810 | C | LYS | 4312 | 13.184 | −11.472 | 45.939 | 1.00 | 37.79 |
| ATOM | 8811 | O | LYS | 4312 | 13.767 | −10.427 | 45.639 | 1.00 | 38.24 |
| ATOM | 8812 | N | THR | 4313 | 12.797 | −11.746 | 47.176 | 1.00 | 37.73 |
| ATOM | 8813 | CA | THR | 4313 | 13.040 | −10.807 | 48.265 | 1.00 | 38.02 |
| ATOM | 8814 | CB | THR | 4313 | 11.747 | −10.058 | 48.619 | 1.00 | 38.54 |
| ATOM | 8815 | OG1 | THR | 4313 | 11.394 | −9.211 | 47.518 | 1.00 | 40.90 |
| ATOM | 8816 | CG2 | THR | 4313 | 11.923 | −9.205 | 49.865 | 1.00 | 38.44 |
| ATOM | 8817 | C | THR | 4313 | 13.601 | −11.481 | 49.513 | 1.00 | 37.68 |
| ATOM | 8818 | O | THR | 4313 | 13.035 | −12.451 | 50.021 | 1.00 | 38.33 |
| ATOM | 8819 | N | ALA | 4314 | 14.718 | −10.960 | 50.006 | 1.00 | 36.78 |
| ATOM | 8820 | CA | ALA | 4314 | 15.343 | −11.519 | 51.194 | 1.00 | 35.56 |
| ATOM | 8821 | CB | ALA | 4314 | 16.682 | −10.847 | 51.457 | 1.00 | 35.27 |
| ATOM | 8822 | C | ALA | 4314 | 14.431 | −11.355 | 52.397 | 1.00 | 34.96 |
| ATOM | 8823 | O | ALA | 4314 | 13.648 | −10.410 | 52.481 | 1.00 | 33.96 |
| ATOM | 8824 | N | GLY | 4315 | 14.545 | −12.297 | 53.322 | 1.00 | 34.82 |
| ATOM | 8825 | CA | GLY | 4315 | 13.743 | −12.276 | 54.527 | 1.00 | 35.43 |
| ATOM | 8826 | C | GLY | 4315 | 13.925 | −13.612 | 55.208 | 1.00 | 36.19 |
| ATOM | 8827 | O | GLY | 4315 | 14.767 | −14.417 | 54.797 | 1.00 | 36.27 |
| ATOM | 8828 | N | VAL | 4316 | 13.136 | −13.863 | 56.238 | 1.00 | 36.45 |
| ATOM | 8829 | CA | VAL | 4316 | 13.234 | −15.116 | 56.960 | 1.00 | 37.25 |
| ATOM | 8830 | CB | VAL | 4316 | 12.273 | −15.115 | 58.134 | 1.00 | 37.67 |
| ATOM | 8831 | CG1 | VAL | 4316 | 12.510 | −16.346 | 58.997 | 1.00 | 39.80 |
| ATOM | 8832 | CG2 | VAL | 4316 | 12.466 | −13.856 | 58.940 | 1.00 | 37.70 |
| ATOM | 8833 | C | VAL | 4316 | 12.935 | −16.339 | 56.094 | 1.00 | 37.44 |
| ATOM | 8834 | O | VAL | 4316 | 13.459 | −17.427 | 56.352 | 1.00 | 37.16 |
| ATOM | 8835 | N | ASN | 4317 | 12.100 | −16.162 | 55.066 | 1.00 | 38.11 |
| ATOM | 8836 | CA | ASN | 4317 | 11.725 | −17.274 | 54.184 | 1.00 | 38.14 |
| ATOM | 8837 | CB | ASN | 4317 | 10.293 | −17.101 | 53.697 | 1.00 | 37.64 |
| ATOM | 8838 | CG | ASN | 4317 | 9.281 | −17.268 | 54.817 | 1.00 | 38.22 |
| ATOM | 8839 | OD1 | ASN | 4317 | 8.152 | −16.788 | 54.725 | 1.00 | 37.85 |
| ATOM | 8840 | ND2 | ASN | 4317 | 9.683 | −17.960 | 55.883 | 1.00 | 38.04 |
| ATOM | 8841 | C | ASN | 4317 | 12.653 | −17.455 | 53.003 | 1.00 | 38.48 |
| ATOM | 8842 | O | ASN | 4317 | 12.670 | −18.508 | 52.372 | 1.00 | 39.03 |
| ATOM | 8843 | N | THR | 4318 | 13.431 | −16.426 | 52.708 | 1.00 | 39.26 |
| ATOM | 8844 | CA | THR | 4318 | 14.383 | −16.477 | 51.604 | 1.00 | 39.67 |
| ATOM | 8845 | CB | THR | 4318 | 13.895 | −15.628 | 50.395 | 1.00 | 39.59 |
| ATOM | 8846 | OG1 | THR | 4318 | 12.480 | −15.411 | 50.485 | 1.00 | 38.61 |
| ATOM | 8847 | CG2 | THR | 4318 | 14.185 | −16.352 | 49.100 | 1.00 | 39.85 |
| ATOM | 8848 | C | THR | 4318 | 15.693 | −15.901 | 52.144 | 1.00 | 39.64 |
| ATOM | 8849 | O | THR | 4318 | 15.984 | −14.719 | 51.981 | 1.00 | 39.63 |
| ATOM | 8850 | N | THR | 4319 | 16.457 | −16.752 | 52.810 | 1.00 | 39.48 |
| ATOM | 8851 | CA | THR | 4319 | 17.725 | −16.396 | 53.412 | 1.00 | 39.02 |
| ATOM | 8852 | CB | THR | 4319 | 18.269 | −17.637 | 54.113 | 1.00 | 38.51 |
| ATOM | 8853 | OG1 | THR | 4319 | 17.585 | −17.777 | 55.358 | 1.00 | 37.16 |
| ATOM | 8854 | CG2 | THR | 4319 | 19.763 | −17.558 | 54.333 | 1.00 | 38.85 |
| ATOM | 8855 | C | THR | 4319 | 18.761 | −15.858 | 52.440 | 1.00 | 39.30 |
| ATOM | 8856 | O | THR | 4319 | 18.664 | −16.076 | 51.240 | 1.00 | 38.65 |
| ATOM | 8857 | N | ASP | 4320 | 19.760 | −15.162 | 52.977 | 1.00 | 40.12 |
| ATOM | 8858 | CA | ASP | 4320 | 20.831 | −14.607 | 52.165 | 1.00 | 41.46 |
| ATOM | 8859 | CB | ASP | 4320 | 21.782 | −13.801 | 53.035 | 1.00 | 42.49 |
| ATOM | 8860 | CG | ASP | 4320 | 21.142 | −12.570 | 53.609 | 1.00 | 43.67 |
| ATOM | 8861 | OD1 | ASP | 4320 | 20.476 | −11.828 | 52.855 | 1.00 | 43.47 |
| ATOM | 8862 | OD2 | ASP | 4320 | 21.322 | −12.337 | 54.822 | 1.00 | 46.01 |
| ATOM | 8863 | C | ASP | 4320 | 21.631 | −15.680 | 51.431 | 1.00 | 41.87 |
| ATOM | 8864 | O | ASP | 4320 | 22.231 | −15.418 | 50.395 | 1.00 | 41.52 |
| ATOM | 8865 | N | LYS | 4321 | 21.649 | −16.888 | 51.977 | 1.00 | 42.88 |
| ATOM | 8866 | CA | LYS | 4321 | 22.383 | −17.989 | 51.363 | 1.00 | 43.85 |
| ATOM | 8867 | CB | LYS | 4321 | 21.990 | −19.316 | 52.020 | 1.00 | 44.70 |
| ATOM | 8868 | CG | LYS | 4321 | 22.217 | −19.370 | 53.524 | 1.00 | 47.07 |
| ATOM | 8869 | CD | LYS | 4321 | 21.400 | −20.502 | 54.162 | 1.00 | 48.07 |
| ATOM | 8870 | CE | LYS | 4321 | 21.161 | −20.260 | 55.666 | 1.00 | 48.55 |
| ATOM | 8871 | NZ | LYS | 4321 | 22.415 | −20.272 | 56.470 | 1.00 | 48.32 |
| ATOM | 8872 | C | LYS | 4321 | 22.076 | −18.084 | 49.872 | 1.00 | 44.10 |
| ATOM | 8873 | O | LYS | 4321 | 22.927 | −18.481 | 49.071 | 1.00 | 43.23 |
| ATOM | 8874 | N | GLU | 4322 | 20.860 | −17.696 | 49.506 | 1.00 | 44.43 |
| ATOM | 8875 | CA | GLU | 4322 | 20.416 | −17.803 | 48.129 | 1.00 | 45.34 |
| ATOM | 8876 | CB | GLU | 4322 | 19.222 | −18.753 | 48.091 | 1.00 | 47.85 |
| ATOM | 8877 | CG | GLU | 4322 | 18.105 | −18.327 | 49.032 | 1.00 | 50.35 |
| ATOM | 8878 | CD | GLU | 4322 | 16.987 | −19.348 | 49.128 | 1.00 | 52.52 |
| ATOM | 8879 | OE1 | GLU | 4322 | 16.206 | −19.502 | 48.154 | 1.00 | 53.64 |
| ATOM | 8880 | OE2 | GLU | 4322 | 16.894 | −20.005 | 50.188 | 1.00 | 54.36 |
| ATOM | 8881 | C | GLU | 4322 | 20.028 | −16.516 | 47.418 | 1.00 | 44.68 |
| ATOM | 8882 | O | GLU | 4322 | 19.521 | −16.567 | 46.298 | 1.00 | 45.07 |
| ATOM | 8883 | N | MSE | 4323 | 20.254 | −15.365 | 48.035 | 1.00 | 43.72 |
| ATOM | 8884 | CA | MSE | 4323 | 19.860 | −14.118 | 47.384 | 1.00 | 42.91 |
| ATOM | 8885 | CB | MSE | 4323 | 19.547 | −13.033 | 48.424 | 1.00 | 43.05 |
| ATOM | 8886 | CG | MSE | 4323 | 18.302 | −13.284 | 49.257 | 1.00 | 42.47 |
| ATOM | 8887 | SE | MSE | 4323 | 16.792 | −13.495 | 48.297 | 1.00 | 42.50 |
| ATOM | 8888 | CE | MSE | 4323 | 16.851 | −12.033 | 47.279 | 1.00 | 43.13 |
| ATOM | 8889 | C | MSE | 4323 | 20.858 | −13.565 | 46.378 | 1.00 | 42.72 |
| ATOM | 8890 | O | MSE | 4323 | 20.529 | −12.654 | 45.624 | 1.00 | 42.58 |
| ATOM | 8891 | N | GLU | 4324 | 22.075 | −14.097 | 46.354 | 1.00 | 42.91 |
| ATOM | 8892 | CA | GLU | 4324 | 23.067 | −13.600 | 45.405 | 1.00 | 42.92 |
| ATOM | 8893 | CB | GLU | 4324 | 24.488 | −13.805 | 45.946 | 1.00 | 43.70 |
| ATOM | 8894 | CG | GLU | 4324 | 24.843 | −12.877 | 47.111 | 1.00 | 45.11 |
| ATOM | 8895 | CD | GLU | 4324 | 26.347 | −12.753 | 47.355 | 1.00 | 45.88 |
| ATOM | 8896 | OE1 | GLU | 4324 | 26.983 | −13.749 | 47.782 | 1.00 | 45.81 |
| ATOM | 8897 | OE2 | GLU | 4324 | 26.888 | −11.647 | 47.116 | 1.00 | 46.44 |
| ATOM | 8898 | C | GLU | 4324 | 22.941 | −14.225 | 44.010 | 1.00 | 42.89 |
| ATOM | 8899 | O | GLU | 4324 | 23.787 | −14.001 | 43.145 | 1.00 | 43.47 |
| ATOM | 8900 | N | VAL | 4325 | 21.884 | −14.995 | 43.778 | 1.00 | 42.26 |
| ATOM | 8901 | CA | VAL | 4325 | 21.702 | −15.599 | 42.471 | 1.00 | 42.10 |
| ATOM | 8902 | CB | VAL | 4325 | 22.334 | −17.013 | 42.415 | 1.00 | 42.51 |
| ATOM | 8903 | CG1 | VAL | 4325 | 21.380 | −18.039 | 42.994 | 1.00 | 42.17 |
| ATOM | 8904 | CG2 | VAL | 4325 | 22.703 | −17.364 | 40.981 | 1.00 | 43.51 |
| ATOM | 8905 | C | VAL | 4325 | 20.222 | −15.678 | 42.112 | 1.00 | 42.11 |
| ATOM | 8906 | O | VAL | 4325 | 19.374 | −15.940 | 42.967 | 1.00 | 42.16 |
| ATOM | 8907 | N | LEU | 4326 | 19.913 | −15.428 | 40.846 | 1.00 | 42.18 |
| ATOM | 8908 | CA | LEU | 4326 | 18.539 | −15.486 | 40.374 | 1.00 | 42.61 |
| ATOM | 8909 | CB | LEU | 4326 | 18.111 | −14.138 | 39.817 | 1.00 | 43.09 |
| ATOM | 8910 | CG | LEU | 4326 | 16.718 | −14.170 | 39.196 | 1.00 | 43.19 |
| ATOM | 8911 | CD1 | LEU | 4326 | 15.708 | −14.549 | 40.267 | 1.00 | 43.61 |
| ATOM | 8912 | CD2 | LEU | 4326 | 16.389 | −12.813 | 38.596 | 1.00 | 43.18 |
| ATOM | 8913 | C | LEU | 4326 | 18.474 | −16.531 | 39.271 | 1.00 | 43.23 |
| ATOM | 8914 | O | LEU | 4326 | 19.187 | −16.424 | 38.267 | 1.00 | 42.91 |
| ATOM | 8915 | N | HIS | 4327 | 17.627 | −17.539 | 39.465 | 1.00 | 43.90 |
| ATOM | 8916 | CA | HIS | 4327 | 17.481 | −18.617 | 38.494 | 1.00 | 44.62 |
| ATOM | 8917 | CB | HIS | 4327 | 17.483 | −19.980 | 39.203 | 1.00 | 45.27 |
| ATOM | 8918 | CG | HIS | 4327 | 18.802 | −20.342 | 39.810 | 1.00 | 45.50 |
| ATOM | 8919 | CD2 | HIS | 4327 | 19.165 | −20.553 | 41.098 | 1.00 | 45.92 |
| ATOM | 8920 | ND1 | HIS | 4327 | 19.946 | −20.507 | 39.057 | 1.00 | 46.35 |
| ATOM | 8921 | CE1 | HIS | 4327 | 20.958 | −20.803 | 39.855 | 1.00 | 46.62 |
| ATOM | 8922 | NE2 | HIS | 4327 | 20.511 | −20.837 | 41.099 | 1.00 | 46.72 |
| ATOM | 8923 | C | HIS | 4327 | 16.217 | −18.477 | 37.657 | 1.00 | 44.86 |
| ATOM | 8924 | O | HIS | 4327 | 15.119 | −18.250 | 38.181 | 1.00 | 44.01 |
| ATOM | 8925 | N | LEU | 4328 | 16.398 | −18.607 | 36.348 | 1.00 | 45.17 |
| ATOM | 8926 | CA | LEU | 4328 | 15.309 | −18.519 | 35.390 | 1.00 | 46.29 |
| ATOM | 8927 | CB | LEU | 4328 | 15.544 | −17.309 | 34.489 | 1.00 | 45.48 |
| ATOM | 8928 | CG | LEU | 4328 | 15.613 | −16.025 | 35.319 | 1.00 | 45.17 |
| ATOM | 8929 | CD1 | LEU | 4328 | 16.213 | −14.884 | 34.515 | 1.00 | 44.75 |
| ATOM | 8930 | CD2 | LEU | 4328 | 14.212 | −15.688 | 35.802 | 1.00 | 44.47 |
| ATOM | 8931 | C | LEU | 4328 | 15.337 | −19.825 | 34.589 | 1.00 | 47.49 |
| ATOM | 8932 | O | LEU | 4328 | 16.347 | −20.153 | 33.953 | 1.00 | 47.87 |
| ATOM | 8933 | N | ARG | 4329 | 14.240 | −20.573 | 34.631 | 1.00 | 48.35 |
| ATOM | 8934 | CA | ARG | 4329 | 14.169 | −21.853 | 33.931 | 1.00 | 49.34 |
| ATOM | 8935 | CB | ARG | 4329 | 13.604 | −22.922 | 34.872 | 1.00 | 50.71 |
| ATOM | 8936 | CG | ARG | 4329 | 14.480 | −23.214 | 36.089 | 1.00 | 52.02 |
| ATOM | 8937 | CD | ARG | 4329 | 15.465 | −24.314 | 35.793 | 1.00 | 53.40 |
| ATOM | 8938 | NE | ARG | 4329 | 16.636 | −24.258 | 36.658 | 1.00 | 54.67 |
| ATOM | 8939 | CZ | ARG | 4329 | 17.687 | −25.061 | 36.538 | 1.00 | 55.58 |
| ATOM | 8940 | NH1 | ARG | 4329 | 17.710 | −25.986 | 35.590 | 1.00 | 55.08 |
| ATOM | 8941 | NH2 | ARG | 4329 | 18.725 | −24.922 | 37.357 | 1.00 | 56.68 |
| ATOM | 8942 | C | ARG | 4329 | 13.315 | −21.801 | 32.671 | 1.00 | 49.42 |
| ATOM | 8943 | O | ARG | 4329 | 12.274 | −21.132 | 32.635 | 1.00 | 49.05 |
| ATOM | 8944 | N | ASN | 4330 | 13.757 | −22.525 | 31.645 | 1.00 | 49.74 |
| ATOM | 8945 | CA | ASN | 4330 | 13.032 | −22.601 | 30.380 | 1.00 | 50.30 |
| ATOM | 8946 | CB | ASN | 4330 | 11.863 | −23.578 | 30.531 | 1.00 | 51.03 |
| ATOM | 8947 | CG | ASN | 4330 | 11.138 | −23.831 | 29.225 | 1.00 | 52.31 |
| ATOM | 8948 | OD1 | ASN | 4330 | 9.960 | −24.208 | 29.219 | 1.00 | 52.11 |
| ATOM | 8949 | ND2 | ASN | 4330 | 11.841 | −23.642 | 28.109 | 1.00 | 52.27 |
| ATOM | 8950 | C | ASN | 4330 | 12.507 | −21.212 | 30.019 | 1.00 | 50.34 |
| ATOM | 8951 | O | ASN | 4330 | 11.292 | −20.966 | 30.034 | 1.00 | 50.09 |
| ATOM | 8952 | N | VAL | 4331 | 13.424 | −20.308 | 29.690 | 1.00 | 50.07 |
| ATOM | 8953 | CA | VAL | 4331 | 13.050 | −18.935 | 29.374 | 1.00 | 50.37 |
| ATOM | 8954 | CB | VAL | 4331 | 14.308 | −18.005 | 29.319 | 1.00 | 50.24 |
| ATOM | 8955 | CG1 | VAL | 4331 | 15.063 | −18.067 | 30.636 | 1.00 | 49.25 |
| ATOM | 8956 | CG2 | VAL | 4331 | 15.221 | −18.407 | 28.168 | 1.00 | 50.36 |
| ATOM | 8957 | C | VAL | 4331 | 12.247 | −18.772 | 28.086 | 1.00 | 50.54 |
| ATOM | 8958 | O | VAL | 4331 | 12.499 | −19.439 | 27.084 | 1.00 | 50.27 |
| ATOM | 8959 | N | SER | 4332 | 11.264 | −17.883 | 28.133 | 1.00 | 50.89 |
| ATOM | 8960 | CA | SER | 4332 | 10.429 | −17.598 | 26.978 | 1.00 | 51.82 |
| ATOM | 8961 | CB | SER | 4332 | 8.953 | −17.595 | 27.387 | 1.00 | 52.23 |
| ATOM | 8962 | OG | SER | 4332 | 8.708 | −16.679 | 28.443 | 1.00 | 52.23 |
| ATOM | 8963 | C | SER | 4332 | 10.847 | −16.226 | 26.460 | 1.00 | 52.24 |
| ATOM | 8964 | O | SER | 4332 | 11.789 | −15.632 | 26.979 | 1.00 | 52.58 |
| ATOM | 8965 | N | PHE | 4333 | 10.167 | −15.722 | 25.435 | 1.00 | 52.88 |
| ATOM | 8966 | CA | PHE | 4333 | 10.515 | −14.410 | 24.903 | 1.00 | 53.24 |
| ATOM | 8967 | CB | PHE | 4333 | 9.925 | −14.225 | 23.499 | 1.00 | 53.47 |
| ATOM | 8968 | CG | PHE | 4333 | 10.707 | −14.938 | 22.430 | 1.00 | 54.45 |
| ATOM | 8969 | CD1 | PHE | 4333 | 10.066 | −15.723 | 21.478 | 1.00 | 54.97 |
| ATOM | 8970 | CD2 | PHE | 4333 | 12.103 | −14.839 | 22.390 | 1.00 | 54.87 |
| ATOM | 8971 | CE1 | PHE | 4333 | 10.799 | −16.401 | 20.501 | 1.00 | 55.17 |
| ATOM | 8972 | CE2 | PHE | 4333 | 12.851 | −15.513 | 21.417 | 1.00 | 54.84 |
| ATOM | 8973 | CZ | PHE | 4333 | 12.199 | −16.295 | 20.470 | 1.00 | 55.41 |
| ATOM | 8974 | C | PHE | 4333 | 10.023 | −13.345 | 25.864 | 1.00 | 53.23 |
| ATOM | 8975 | O | PHE | 4333 | 10.498 | −12.208 | 25.854 | 1.00 | 53.76 |
| ATOM | 8976 | N | GLU | 4334 | 9.082 | −13.741 | 26.714 | 1.00 | 53.25 |
| ATOM | 8977 | CA | GLU | 4334 | 8.505 | −12.858 | 27.718 | 1.00 | 53.69 |
| ATOM | 8978 | CB | GLU | 4334 | 7.313 | −13.547 | 28.405 | 1.00 | 55.25 |
| ATOM | 8979 | CG | GLU | 4334 | 6.222 | −14.105 | 27.473 | 1.00 | 58.21 |
| ATOM | 8980 | CD | GLU | 4334 | 6.632 | −15.385 | 26.728 | 1.00 | 59.31 |
| ATOM | 8981 | OE1 | GLU | 4334 | 7.535 | −15.324 | 25.865 | 1.00 | 59.75 |
| ATOM | 8982 | OE2 | GLU | 4334 | 6.041 | −16.457 | 27.003 | 1.00 | 59.89 |
| ATOM | 8983 | C | GLU | 4334 | 9.569 | −12.549 | 28.774 | 1.00 | 52.54 |
| ATOM | 8984 | O | GLU | 4334 | 9.624 | −11.447 | 29.326 | 1.00 | 52.53 |
| ATOM | 8985 | N | ASP | 4335 | 10.410 | −13.541 | 29.047 | 1.00 | 50.75 |
| ATOM | 8986 | CA | ASP | 4335 | 11.458 | −13.408 | 30.046 | 1.00 | 49.35 |
| ATOM | 8987 | CB | ASP | 4335 | 12.096 | −14.774 | 30.333 | 1.00 | 50.63 |
| ATOM | 8988 | CG | ASP | 4335 | 11.170 | −15.703 | 31.105 | 1.00 | 51.28 |
| ATOM | 8989 | OD1 | ASP | 4335 | 10.589 | −15.240 | 32.115 | 1.00 | 50.92 |
| ATOM | 8990 | OD2 | ASP | 4335 | 11.035 | −16.887 | 30.706 | 1.00 | 51.26 |
| ATOM | 8991 | C | ASP | 4335 | 12.554 | −12.419 | 29.671 | 1.00 | 47.79 |
| ATOM | 8992 | O | ASP | 4335 | 13.271 | −11.923 | 30.543 | 1.00 | 46.93 |
| ATOM | 8993 | N | ALA | 4336 | 12.703 | −12.142 | 28.380 | 1.00 | 45.80 |
| ATOM | 8994 | CA | ALA | 4336 | 13.735 | −11.203 | 27.955 | 1.00 | 43.59 |
| ATOM | 8995 | CB | ALA | 4336 | 13.773 | −11.101 | 26.434 | 1.00 | 43.74 |
| ATOM | 8996 | C | ALA | 4336 | 13.447 | −9.838 | 28.581 | 1.00 | 42.27 |
| ATOM | 8997 | O | ALA | 4336 | 12.295 | −9.518 | 28.912 | 1.00 | 41.75 |
| ATOM | 8998 | N | GLY | 4337 | 14.494 | −9.042 | 28.760 | 1.00 | 40.54 |
| ATOM | 8999 | CA | GLY | 4337 | 14.312 | −7.729 | 29.353 | 1.00 | 39.10 |
| ATOM | 9000 | C | GLY | 4337 | 15.382 | −7.351 | 30.358 | 1.00 | 38.15 |
| ATOM | 9001 | O | GLY | 4337 | 16.441 | −7.979 | 30.433 | 1.00 | 37.94 |
| ATOM | 9002 | N | GLU | 4338 | 15.096 | −6.324 | 31.148 | 1.00 | 37.17 |
| ATOM | 9003 | CA | GLU | 4338 | 16.052 | −5.851 | 32.137 | 1.00 | 36.63 |
| ATOM | 9004 | CB | GLU | 4338 | 16.082 | −4.320 | 32.139 | 1.00 | 37.34 |
| ATOM | 9005 | CG | GLU | 4338 | 17.145 | −3.728 | 33.041 | 1.00 | 38.63 |
| ATOM | 9006 | CD | GLU | 4338 | 17.414 | −2.257 | 32.751 | 1.00 | 38.86 |
| ATOM | 9007 | OE1 | GLU | 4338 | 16.449 | −1.456 | 32.748 | 1.00 | 38.07 |
| ATOM | 9008 | OE2 | GLU | 4338 | 18.597 | −1.909 | 32.537 | 1.00 | 37.89 |
| ATOM | 9009 | C | GLU | 4338 | 15.790 | −6.367 | 33.543 | 1.00 | 35.90 |
| ATOM | 9010 | O | GLU | 4338 | 14.671 | −6.283 | 34.060 | 1.00 | 35.69 |
| ATOM | 9011 | N | TYR | 4339 | 16.837 | −6.917 | 34.149 | 1.00 | 34.68 |
| ATOM | 9012 | CA | TYR | 4339 | 16.759 | −7.445 | 35.504 | 1.00 | 34.15 |
| ATOM | 9013 | CB | TYR | 4339 | 17.209 | −8.912 | 35.552 | 1.00 | 34.74 |
| ATOM | 9014 | CG | TYR | 4339 | 16.268 | −9.869 | 34.860 | 1.00 | 36.20 |
| ATOM | 9015 | CD1 | TYR | 4339 | 16.379 | −10.119 | 33.488 | 1.00 | 36.21 |
| ATOM | 9016 | CE1 | TYR | 4339 | 15.480 | −10.950 | 32.831 | 1.00 | 36.19 |
| ATOM | 9017 | CD2 | TYR | 4339 | 15.228 | −10.486 | 35.563 | 1.00 | 36.48 |
| ATOM | 9018 | CE2 | TYR | 4339 | 14.317 | −11.320 | 34.913 | 1.00 | 37.17 |
| ATOM | 9019 | CZ | TYR | 4339 | 14.452 | −11.544 | 33.544 | 1.00 | 37.10 |
| ATOM | 9020 | OH | TYR | 4339 | 13.548 | −12.347 | 32.889 | 1.00 | 37.74 |
| ATOM | 9021 | C | TYR | 4339 | 17.648 | −6.607 | 36.418 | 1.00 | 33.66 |
| ATOM | 9022 | O | TYR | 4339 | 18.766 | −6.230 | 36.051 | 1.00 | 33.45 |
| ATOM | 9023 | N | THR | 4340 | 17.147 | −6.322 | 37.616 | 1.00 | 32.86 |
| ATOM | 9024 | CA | THR | 4340 | 17.889 | −5.513 | 38.573 | 1.00 | 31.96 |
| ATOM | 9025 | CB | THR | 4340 | 17.167 | −4.165 | 38.864 | 1.00 | 31.39 |
| ATOM | 9026 | OG1 | THR | 4340 | 17.178 | −3.348 | 37.696 | 1.00 | 31.59 |
| ATOM | 9027 | CG2 | THR | 4340 | 17.854 | −3.414 | 39.992 | 1.00 | 30.92 |
| ATOM | 9028 | C | THR | 4340 | 18.105 | −6.176 | 39.924 | 1.00 | 31.77 |
| ATOM | 9029 | O | THR | 4340 | 17.217 | −6.822 | 40.469 | 1.00 | 29.88 |
| ATOM | 9030 | N | CYS | 4341 | 19.306 | −5.986 | 40.456 | 1.00 | 32.88 |
| ATOM | 9031 | CA | CYS | 4341 | 19.654 | −6.479 | 41.771 | 1.00 | 33.28 |
| ATOM | 9032 | CB | CYS | 4341 | 21.020 | −7.142 | 41.763 | 1.00 | 34.10 |
| ATOM | 9033 | SG | CYS | 4341 | 21.407 | −7.752 | 43.407 | 1.00 | 39.81 |
| ATOM | 9034 | C | CYS | 4341 | 19.702 | −5.233 | 42.657 | 1.00 | 33.07 |
| ATOM | 9035 | O | CYS | 4341 | 20.601 | −4.391 | 42.522 | 1.00 | 33.07 |
| ATOM | 9036 | N | LEU | 4342 | 18.726 | −5.108 | 43.545 | 1.00 | 32.27 |
| ATOM | 9037 | CA | LEU | 4342 | 18.651 | −3.966 | 44.438 | 1.00 | 31.87 |
| ATOM | 9038 | CB | LEU | 4342 | 17.221 | −3.435 | 44.475 | 1.00 | 32.99 |
| ATOM | 9039 | CG | LEU | 4342 | 17.035 | −2.098 | 45.196 | 1.00 | 33.58 |
| ATOM | 9040 | CD1 | LEU | 4342 | 17.196 | −0.963 | 44.204 | 1.00 | 33.18 |
| ATOM | 9041 | CD2 | LEU | 4342 | 15.662 | −2.041 | 45.814 | 1.00 | 33.96 |
| ATOM | 9042 | C | LEU | 4342 | 19.097 | −4.342 | 45.855 | 1.00 | 31.36 |
| ATOM | 9043 | O | LEU | 4342 | 18.636 | −5.322 | 46.423 | 1.00 | 31.33 |
| ATOM | 9044 | N | ALA | 4343 | 19.991 | −3.548 | 46.428 | 1.00 | 30.62 |
| ATOM | 9045 | CA | ALA | 4343 | 20.498 | −3.811 | 47.770 | 1.00 | 29.61 |
| ATOM | 9046 | CB | ALA | 4343 | 21.915 | −4.361 | 47.682 | 1.00 | 29.93 |
| ATOM | 9047 | C | ALA | 4343 | 20.510 | −2.551 | 48.607 | 1.00 | 28.83 |
| ATOM | 9048 | O | ALA | 4343 | 21.034 | −1.531 | 48.181 | 1.00 | 29.09 |
| ATOM | 9049 | N | GLY | 4344 | 19.954 | −2.612 | 49.805 | 1.00 | 28.42 |
| ATOM | 9050 | CA | GLY | 4344 | 19.969 | −1.427 | 50.636 | 1.00 | 28.20 |
| ATOM | 9051 | C | GLY | 4344 | 20.132 | −1.701 | 52.115 | 1.00 | 27.80 |
| ATOM | 9052 | O | GLY | 4344 | 19.922 | −2.820 | 52.573 | 1.00 | 27.63 |
| ATOM | 9053 | N | ASN | 4345 | 20.545 | −0.676 | 52.853 | 1.00 | 27.69 |
| ATOM | 9054 | CA | ASN | 4345 | 20.689 | −0.759 | 54.301 | 1.00 | 27.76 |
| ATOM | 9055 | CB | ASN | 4345 | 22.131 | −1.098 | 54.733 | 1.00 | 26.42 |
| ATOM | 9056 | CG | ASN | 4345 | 23.167 | −0.101 | 54.231 | 1.00 | 26.03 |
| ATOM | 9057 | OD1 | ASN | 4345 | 22.911 | 1.105 | 54.169 | 1.00 | 26.17 |
| ATOM | 9058 | ND2 | ASN | 4345 | 24.358 | −0.600 | 53.898 | 1.00 | 23.59 |
| ATOM | 9059 | C | ASN | 4345 | 20.253 | 0.602 | 54.816 | 1.00 | 28.42 |
| ATOM | 9060 | O | ASN | 4345 | 19.896 | 1.467 | 54.032 | 1.00 | 29.18 |
| ATOM | 9061 | N | SER | 4346 | 20.269 | 0.803 | 56.121 | 1.00 | 29.94 |
| ATOM | 9062 | CA | SER | 4346 | 19.820 | 2.075 | 56.676 | 1.00 | 30.91 |
| ATOM | 9063 | CB | SER | 4346 | 20.074 | 2.091 | 58.178 | 1.00 | 31.28 |
| ATOM | 9064 | OG | SER | 4346 | 21.283 | 1.408 | 58.486 | 1.00 | 34.27 |
| ATOM | 9065 | C | SER | 4346 | 20.461 | 3.287 | 56.010 | 1.00 | 31.02 |
| ATOM | 9066 | O | SER | 4346 | 19.808 | 4.312 | 55.807 | 1.00 | 30.63 |
| ATOM | 9067 | N | ILE | 4347 | 21.726 | 3.142 | 55.633 | 1.00 | 31.12 |
| ATOM | 9068 | CA | ILE | 4347 | 22.491 | 4.218 | 55.009 | 1.00 | 31.11 |
| ATOM | 9069 | CB | ILE | 4347 | 23.995 | 3.876 | 55.068 | 1.00 | 31.32 |
| ATOM | 9070 | CG2 | ILE | 4347 | 24.815 | 5.044 | 54.556 | 1.00 | 30.06 |
| ATOM | 9071 | CG1 | ILE | 4347 | 24.375 | 3.498 | 56.502 | 1.00 | 29.98 |
| ATOM | 9072 | CD1 | ILE | 4347 | 25.748 | 2.862 | 56.626 | 1.00 | 30.91 |
| ATOM | 9073 | C | ILE | 4347 | 22.136 | 4.583 | 53.563 | 1.00 | 30.91 |
| ATOM | 9074 | O | ILE | 4347 | 22.216 | 5.752 | 53.194 | 1.00 | 31.96 |
| ATOM | 9075 | N | GLY | 4348 | 21.763 | 3.596 | 52.746 | 1.00 | 30.75 |
| ATOM | 9076 | CA | GLY | 4348 | 21.434 | 3.884 | 51.357 | 1.00 | 29.90 |
| ATOM | 9077 | C | GLY | 4348 | 21.122 | 2.691 | 50.467 | 1.00 | 30.05 |
| ATOM | 9078 | O | GLY | 4348 | 21.173 | 1.546 | 50.905 | 1.00 | 30.56 |
| ATOM | 9079 | N | LEU | 4349 | 20.803 | 2.982 | 49.206 | 1.00 | 29.94 |
| ATOM | 9080 | CA | LEU | 4349 | 20.447 | 1.979 | 48.204 | 1.00 | 30.28 |
| ATOM | 9081 | CB | LEU | 4349 | 19.079 | 2.316 | 47.599 | 1.00 | 30.79 |
| ATOM | 9082 | CG | LEU | 4349 | 17.813 | 1.980 | 48.404 | 1.00 | 33.29 |
| ATOM | 9083 | CD1 | LEU | 4349 | 17.773 | 0.465 | 48.616 | 1.00 | 33.89 |
| ATOM | 9084 | CD2 | LEU | 4349 | 17.775 | 2.703 | 49.746 | 1.00 | 32.39 |
| ATOM | 9085 | C | LEU | 4349 | 21.458 | 1.865 | 47.065 | 1.00 | 30.14 |
| ATOM | 9086 | O | LEU | 4349 | 22.099 | 2.842 | 46.690 | 1.00 | 29.38 |
| ATOM | 9087 | N | SER | 4350 | 21.580 | 0.667 | 46.505 | 1.00 | 29.74 |
| ATOM | 9088 | CA | SER | 4350 | 22.493 | 0.432 | 45.395 | 1.00 | 29.96 |
| ATOM | 9089 | CB | SER | 4350 | 23.829 | −0.135 | 45.893 | 1.00 | 29.96 |
| ATOM | 9090 | OG | SER | 4350 | 24.543 | 0.797 | 46.683 | 1.00 | 30.04 |
| ATOM | 9091 | C | SER | 4350 | 21.846 | −0.573 | 44.468 | 1.00 | 30.13 |
| ATOM | 9092 | O | SER | 4350 | 21.034 | −1.388 | 44.907 | 1.00 | 30.49 |
| ATOM | 9093 | N | HIS | 4351 | 22.188 | −0.520 | 43.186 | 1.00 | 29.92 |
| ATOM | 9094 | CA | HIS | 4351 | 21.628 | −1.476 | 42.252 | 1.00 | 29.93 |
| ATOM | 9095 | CB | HIS | 4351 | 20.200 | −1.114 | 41.905 | 1.00 | 29.39 |
| ATOM | 9096 | CG | HIS | 4351 | 20.065 | 0.208 | 41.230 | 1.00 | 30.24 |
| ATOM | 9097 | CD2 | HIS | 4351 | 19.885 | 0.527 | 39.927 | 1.00 | 30.19 |
| ATOM | 9098 | ND1 | HIS | 4351 | 20.082 | 1.401 | 41.921 | 1.00 | 30.70 |
| ATOM | 9099 | CE1 | HIS | 4351 | 19.912 | 2.399 | 41.072 | 1.00 | 29.67 |
| ATOM | 9100 | NE2 | HIS | 4351 | 19.790 | 1.895 | 39.857 | 1.00 | 29.36 |
| ATOM | 9101 | C | HIS | 4351 | 22.429 | −1.594 | 40.980 | 1.00 | 30.48 |
| ATOM | 9102 | O | HIS | 4351 | 23.105 | −0.653 | 40.564 | 1.00 | 31.12 |
| ATOM | 9103 | N | HIS | 4352 | 22.362 | −2.781 | 40.384 | 1.00 | 30.52 |
| ATOM | 9104 | CA | HIS | 4352 | 23.039 | −3.092 | 39.129 | 1.00 | 30.07 |
| ATOM | 9105 | CB | HIS | 4352 | 24.148 | −4.112 | 39.339 | 1.00 | 29.99 |
| ATOM | 9106 | CG | HIS | 4352 | 25.465 | −3.508 | 39.685 | 1.00 | 31.26 |
| ATOM | 9107 | CD2 | HIS | 4352 | 25.881 | −2.218 | 39.684 | 1.00 | 31.91 |
| ATOM | 9108 | ND1 | HIS | 4352 | 26.539 | −4.260 | 40.108 | 1.00 | 31.32 |
| ATOM | 9109 | CE1 | HIS | 4352 | 27.561 | −3.460 | 40.360 | 1.00 | 32.79 |
| ATOM | 9110 | NE2 | HIS | 4352 | 27.187 | −2.215 | 40.110 | 1.00 | 33.25 |
| ATOM | 9111 | C | HIS | 4352 | 21.973 | −3.721 | 38.253 | 1.00 | 30.31 |
| ATOM | 9112 | O | HIS | 4352 | 21.079 | −4.407 | 38.755 | 1.00 | 30.36 |
| ATOM | 9113 | N | SER | 4353 | 22.062 | −3.494 | 36.950 | 1.00 | 29.99 |
| ATOM | 9114 | CA | SER | 4353 | 21.090 | −4.049 | 36.022 | 1.00 | 29.61 |
| ATOM | 9115 | CB | SER | 4353 | 20.229 | −2.942 | 35.402 | 1.00 | 29.98 |
| ATOM | 9116 | OG | SER | 4353 | 19.357 | −2.357 | 36.355 | 1.00 | 30.66 |
| ATOM | 9117 | C | SER | 4353 | 21.794 | −4.785 | 34.912 | 1.00 | 29.84 |
| ATOM | 9118 | O | SER | 4353 | 22.948 | −4.517 | 34.601 | 1.00 | 29.86 |
| ATOM | 9119 | N | ALA | 4354 | 21.089 | −5.728 | 34.315 | 1.00 | 30.68 |
| ATOM | 9120 | CA | ALA | 4354 | 21.642 | −6.482 | 33.213 | 1.00 | 32.16 |
| ATOM | 9121 | CB | ALA | 4354 | 22.259 | −7.785 | 33.704 | 1.00 | 32.55 |
| ATOM | 9122 | C | ALA | 4354 | 20.494 | −6.765 | 32.270 | 1.00 | 33.31 |
| ATOM | 9123 | O | ALA | 4354 | 19.324 | −6.759 | 32.669 | 1.00 | 32.63 |
| ATOM | 9124 | N | TRP | 4355 | 20.829 | −6.995 | 31.010 | 1.00 | 34.78 |
| ATOM | 9125 | CA | TRP | 4355 | 19.812 | −7.291 | 30.021 | 1.00 | 36.62 |
| ATOM | 9126 | CB | TRP | 4355 | 20.001 | −6.417 | 28.779 | 1.00 | 39.13 |
| ATOM | 9127 | CG | TRP | 4355 | 18.771 | −5.629 | 28.485 | 1.00 | 42.31 |
| ATOM | 9128 | CD2 | TRP | 4355 | 18.533 | −4.272 | 28.852 | 1.00 | 43.06 |
| ATOM | 9129 | CE2 | TRP | 4355 | 17.193 | −3.968 | 28.493 | 1.00 | 43.78 |
| ATOM | 9130 | CE3 | TRP | 4355 | 19.317 | −3.279 | 29.455 | 1.00 | 42.62 |
| ATOM | 9131 | CD1 | TRP | 4355 | 17.604 | −6.088 | 27.923 | 1.00 | 43.18 |
| ATOM | 9132 | NE1 | TRP | 4355 | 16.652 | −5.095 | 27.929 | 1.00 | 43.64 |
| ATOM | 9133 | CZ2 | TRP | 4355 | 16.624 | −2.714 | 28.717 | 1.00 | 44.04 |
| ATOM | 9134 | CZ3 | TRP | 4355 | 18.756 | −2.037 | 29.679 | 1.00 | 44.74 |
| ATOM | 9135 | CH2 | TRP | 4355 | 17.416 | −1.760 | 29.309 | 1.00 | 44.65 |
| ATOM | 9136 | C | TRP | 4355 | 19.849 | −8.759 | 29.621 | 1.00 | 36.51 |
| ATOM | 9137 | O | TRP | 4355 | 20.925 | −9.351 | 29.460 | 1.00 | 36.52 |
| ATOM | 9138 | N | LEU | 4356 | 18.670 | −9.343 | 29.468 | 1.00 | 35.94 |
| ATOM | 9139 | CA | LEU | 4356 | 18.578 | −10.733 | 29.068 | 1.00 | 36.68 |
| ATOM | 9140 | CB | LEU | 4356 | 17.661 | −11.489 | 30.040 | 1.00 | 36.27 |
| ATOM | 9141 | CG | LEU | 4356 | 17.500 | −13.014 | 30.003 | 1.00 | 35.09 |
| ATOM | 9142 | CD1 | LEU | 4356 | 16.069 | −13.334 | 29.649 | 1.00 | 35.03 |
| ATOM | 9143 | CD2 | LEU | 4356 | 18.483 | −13.653 | 29.037 | 1.00 | 34.52 |
| ATOM | 9144 | C | LEU | 4356 | 18.022 | −10.765 | 27.644 | 1.00 | 36.98 |
| ATOM | 9145 | O | LEU | 4356 | 16.918 | −10.286 | 27.391 | 1.00 | 36.45 |
| ATOM | 9146 | N | THR | 4357 | 18.809 | −11.305 | 26.717 | 1.00 | 37.76 |
| ATOM | 9147 | CA | THR | 4357 | 18.395 | −11.402 | 25.317 | 1.00 | 39.53 |
| ATOM | 9148 | CB | THR | 4357 | 19.530 | −10.920 | 24.360 | 1.00 | 40.26 |
| ATOM | 9149 | OG1 | THR | 4357 | 19.758 | −9.516 | 24.549 | 1.00 | 41.24 |
| ATOM | 9150 | CG2 | THR | 4357 | 19.159 | −11.177 | 22.905 | 1.00 | 40.07 |
| ATOM | 9151 | C | THR | 4357 | 18.044 | −12.855 | 24.989 | 1.00 | 39.83 |
| ATOM | 9152 | O | THR | 4357 | 18.871 | −13.752 | 25.158 | 1.00 | 39.54 |
| ATOM | 9153 | N | VAL | 4358 | 16.818 | −13.082 | 24.527 | 1.00 | 40.67 |
| ATOM | 9154 | CA | VAL | 4358 | 16.385 | −14.436 | 24.182 | 1.00 | 41.98 |
| ATOM | 9155 | CB | VAL | 4358 | 15.070 | −14.821 | 24.897 | 1.00 | 42.07 |
| ATOM | 9156 | CG1 | VAL | 4358 | 14.811 | −16.301 | 24.697 | 1.00 | 42.58 |
| ATOM | 9157 | CG2 | VAL | 4358 | 15.151 | −14.484 | 26.397 | 1.00 | 41.10 |
| ATOM | 9158 | C | VAL | 4358 | 16.189 | −14.621 | 22.675 | 1.00 | 42.68 |
| ATOM | 9159 | O | VAL | 4358 | 15.486 | −13.842 | 22.024 | 1.00 | 42.98 |
| ATOM | 9160 | N | LEU | 4359 | 16.824 | −15.653 | 22.125 | 1.00 | 43.36 |
| ATOM | 9161 | CA | LEU | 4359 | 16.713 | −15.951 | 20.704 | 1.00 | 43.69 |
| ATOM | 9162 | CB | LEU | 4359 | 18.098 | −15.973 | 20.069 | 1.00 | 43.39 |
| ATOM | 9163 | CG | LEU | 4359 | 19.024 | −14.825 | 20.459 | 1.00 | 43.23 |
| ATOM | 9164 | CD1 | LEU | 4359 | 20.393 | −15.089 | 19.855 | 1.00 | 42.32 |
| ATOM | 9165 | CD2 | LEU | 4359 | 18.449 | −13.484 | 19.996 | 1.00 | 42.16 |
| ATOM | 9166 | C | LEU | 4359 | 16.032 | −17.312 | 20.514 | 1.00 | 44.77 |
| ATOM | 9167 | O | LEU | 4359 | 15.610 | −17.602 | 19.365 | 1.00 | 46.08 |
| ATOM | 9168 | CB | MSE | 5149 | 36.059 | 22.402 | 111.078 | 1.00 | 75.42 |
| ATOM | 9169 | CG | MSE | 5149 | 36.860 | 23.705 | 110.971 | 1.00 | 78.76 |
| ATOM | 9170 | SE | MSE | 5149 | 37.129 | 24.576 | 112.571 | 1.00 | 83.61 |
| ATOM | 9171 | CE | MSE | 5149 | 38.908 | 24.179 | 112.896 | 1.00 | 81.52 |
| ATOM | 9172 | C | MSE | 5149 | 34.158 | 22.843 | 109.510 | 1.00 | 71.79 |
| ATOM | 9173 | O | MSE | 5149 | 34.856 | 22.409 | 108.592 | 1.00 | 71.61 |
| ATOM | 9174 | N | MSE | 5149 | 33.861 | 21.325 | 111.432 | 1.00 | 72.73 |
| ATOM | 9175 | CA | MSE | 5149 | 34.539 | 22.569 | 110.968 | 1.00 | 73.11 |
| ATOM | 9176 | N | PRO | 5150 | 33.043 | 23.567 | 109.285 | 1.00 | 70.52 |
| ATOM | 9177 | CD | PRO | 5150 | 32.179 | 24.130 | 110.337 | 1.00 | 70.27 |
| ATOM | 9178 | CA | PRO | 5150 | 32.530 | 23.921 | 107.950 | 1.00 | 69.22 |
| ATOM | 9179 | CB | PRO | 5150 | 31.287 | 24.765 | 108.262 | 1.00 | 69.59 |
| ATOM | 9180 | CG | PRO | 5150 | 31.570 | 25.314 | 109.635 | 1.00 | 70.32 |
| ATOM | 9181 | C | PRO | 5150 | 33.518 | 24.642 | 107.030 | 1.00 | 67.89 |
| ATOM | 9182 | O | PRO | 5150 | 34.096 | 25.671 | 107.396 | 1.00 | 67.67 |
| ATOM | 9183 | N | VAL | 5151 | 33.696 | 24.087 | 105.831 | 1.00 | 66.29 |
| ATOM | 9184 | CA | VAL | 5151 | 34.613 | 24.644 | 104.838 | 1.00 | 64.43 |
| ATOM | 9185 | CB | VAL | 5151 | 35.904 | 23.786 | 104.721 | 1.00 | 65.08 |
| ATOM | 9186 | CG1 | VAL | 5151 | 36.898 | 24.462 | 103.790 | 1.00 | 65.10 |
| ATOM | 9187 | CG2 | VAL | 5151 | 36.522 | 23.573 | 106.096 | 1.00 | 64.97 |
| ATOM | 9188 | C | VAL | 5151 | 33.953 | 24.695 | 103.464 | 1.00 | 62.93 |
| ATOM | 9189 | O | VAL | 5151 | 33.521 | 23.668 | 102.935 | 1.00 | 63.17 |
| ATOM | 9190 | N | ALA | 5152 | 33.875 | 25.893 | 102.892 | 1.00 | 61.02 |
| ATOM | 9191 | CA | ALA | 5152 | 33.283 | 26.068 | 101.573 | 1.00 | 59.14 |
| ATOM | 9192 | CB | ALA | 5152 | 33.129 | 27.553 | 101.261 | 1.00 | 59.49 |
| ATOM | 9193 | C | ALA | 5152 | 34.207 | 25.399 | 100.557 | 1.00 | 57.59 |
| ATOM | 9194 | O | ALA | 5152 | 35.428 | 25.460 | 100.690 | 1.00 | 57.49 |
| ATOM | 9195 | N | PRO | 5153 | 33.631 | 24.768 | 99.522 | 1.00 | 56.28 |
| ATOM | 9196 | CD | PRO | 5153 | 32.217 | 24.951 | 99.143 | 1.00 | 56.11 |
| ATOM | 9197 | CA | PRO | 5153 | 34.370 | 24.067 | 98.462 | 1.00 | 55.06 |
| ATOM | 9198 | CB | PRO | 5153 | 33.285 | 23.781 | 97.424 | 1.00 | 55.51 |
| ATOM | 9199 | CG | PRO | 5153 | 32.285 | 24.898 | 97.650 | 1.00 | 55.82 |
| ATOM | 9200 | C | PRO | 5153 | 35.570 | 24.805 | 97.863 | 1.00 | 53.80 |
| ATOM | 9201 | O | PRO | 5153 | 35.512 | 26.010 | 97.617 | 1.00 | 53.63 |
| ATOM | 9202 | N | TYR | 5154 | 36.651 | 24.064 | 97.630 | 1.00 | 52.38 |
| ATOM | 9203 | CA | TYR | 5154 | 37.868 | 24.618 | 97.048 | 1.00 | 51.57 |
| ATOM | 9204 | CB | TYR | 5154 | 38.797 | 25.137 | 98.151 | 1.00 | 51.78 |
| ATOM | 9205 | CG | TYR | 5154 | 39.291 | 24.075 | 99.107 | 1.00 | 51.82 |
| ATOM | 9206 | CD1 | TYR | 5154 | 38.446 | 23.521 | 100.067 | 1.00 | 52.13 |
| ATOM | 9207 | CE1 | TYR | 5154 | 38.899 | 22.529 | 100.943 | 1.00 | 52.75 |
| ATOM | 9208 | CD2 | TYR | 5154 | 40.605 | 23.615 | 99.042 | 1.00 | 52.16 |
| ATOM | 9209 | CE2 | TYR | 5154 | 41.072 | 22.626 | 99.908 | 1.00 | 52.69 |
| ATOM | 9210 | CZ | TYR | 5154 | 40.214 | 22.084 | 100.856 | 1.00 | 53.31 |
| ATOM | 9211 | OH | TYR | 5154 | 40.670 | 21.085 | 101.698 | 1.00 | 54.40 |
| ATOM | 9212 | C | TYR | 5154 | 38.593 | 23.552 | 96.218 | 1.00 | 51.05 |
| ATOM | 9213 | O | TYR | 5154 | 38.495 | 22.355 | 96.504 | 1.00 | 50.78 |
| ATOM | 9214 | N | TRP | 5155 | 39.322 | 23.981 | 95.193 | 1.00 | 50.18 |
| ATOM | 9215 | CA | TRP | 5155 | 40.038 | 23.029 | 94.358 | 1.00 | 49.75 |
| ATOM | 9216 | CB | TRP | 5155 | 40.488 | 23.685 | 93.054 | 1.00 | 48.97 |
| ATOM | 9217 | CG | TRP | 5155 | 39.421 | 24.505 | 92.385 | 1.00 | 48.00 |
| ATOM | 9218 | CD2 | TRP | 5155 | 38.188 | 24.036 | 91.811 | 1.00 | 47.50 |
| ATOM | 9219 | CE2 | TRP | 5155 | 37.530 | 25.159 | 91.262 | 1.00 | 47.56 |
| ATOM | 9220 | CE3 | TRP | 5155 | 37.577 | 22.777 | 91.705 | 1.00 | 47.39 |
| ATOM | 9221 | CD1 | TRP | 5155 | 39.450 | 25.851 | 92.171 | 1.00 | 47.75 |
| ATOM | 9222 | NE1 | TRP | 5155 | 38.324 | 26.252 | 91.498 | 1.00 | 47.87 |
| ATOM | 9223 | CZ2 | TRP | 5155 | 36.285 | 25.066 | 90.610 | 1.00 | 46.86 |
| ATOM | 9224 | CZ3 | TRP | 5155 | 36.335 | 22.684 | 91.056 | 1.00 | 47.18 |
| ATOM | 9225 | CH2 | TRP | 5155 | 35.707 | 23.827 | 90.519 | 1.00 | 46.37 |
| ATOM | 9226 | C | TRP | 5155 | 41.241 | 22.525 | 95.134 | 1.00 | 50.17 |
| ATOM | 9227 | O | TRP | 5155 | 41.912 | 23.294 | 95.820 | 1.00 | 50.09 |
| ATOM | 9228 | N | THR | 5156 | 41.502 | 21.228 | 95.038 | 1.00 | 50.85 |
| ATOM | 9229 | CA | THR | 5156 | 42.625 | 20.637 | 95.741 | 1.00 | 51.79 |
| ATOM | 9230 | CB | THR | 5156 | 42.260 | 19.260 | 96.324 | 1.00 | 52.00 |
| ATOM | 9231 | OG1 | THR | 5156 | 41.902 | 18.366 | 95.262 | 1.00 | 52.03 |
| ATOM | 9232 | CG2 | THR | 5156 | 41.090 | 19.390 | 97.297 | 1.00 | 51.95 |
| ATOM | 9233 | C | THR | 5156 | 43.827 | 20.479 | 94.825 | 1.00 | 53.11 |
| ATOM | 9234 | O | THR | 5156 | 44.961 | 20.374 | 95.294 | 1.00 | 53.90 |
| ATOM | 9235 | N | SER | 5157 | 43.581 | 20.467 | 93.519 | 1.00 | 54.02 |
| ATOM | 9236 | CA | SER | 5157 | 44.652 | 20.314 | 92.542 | 1.00 | 55.05 |
| ATOM | 9237 | CB | SER | 5157 | 44.674 | 18.876 | 92.014 | 1.00 | 55.32 |
| ATOM | 9238 | OG | SER | 5157 | 44.910 | 17.948 | 93.063 | 1.00 | 56.15 |
| ATOM | 9239 | C | SER | 5157 | 44.465 | 21.278 | 91.383 | 1.00 | 55.44 |
| ATOM | 9240 | O | SER | 5157 | 44.398 | 20.860 | 90.231 | 1.00 | 55.47 |
| ATOM | 9241 | N | PRO | 5158 | 44.377 | 22.584 | 91.674 | 1.00 | 56.06 |
| ATOM | 9242 | CD | PRO | 5158 | 44.505 | 23.222 | 92.994 | 1.00 | 56.15 |
| ATOM | 9243 | CA | PRO | 5158 | 44.197 | 23.587 | 90.619 | 1.00 | 56.96 |
| ATOM | 9244 | CB | PRO | 5158 | 44.268 | 24.915 | 91.383 | 1.00 | 56.90 |
| ATOM | 9245 | CG | PRO | 5158 | 45.051 | 24.566 | 92.627 | 1.00 | 56.66 |
| ATOM | 9246 | C | PRO | 5158 | 45.257 | 23.465 | 89.520 | 1.00 | 57.69 |
| ATOM | 9247 | O | PRO | 5158 | 45.042 | 23.881 | 88.377 | 1.00 | 57.91 |
| ATOM | 9248 | N | GLU | 5159 | 46.394 | 22.876 | 89.875 | 1.00 | 58.31 |
| ATOM | 9249 | CA | GLU | 5159 | 47.488 | 22.676 | 88.926 | 1.00 | 58.87 |
| ATOM | 9250 | CB | GLU | 5159 | 48.687 | 21.999 | 89.596 | 1.00 | 59.06 |
| ATOM | 9251 | CG | GLU | 5159 | 49.257 | 22.713 | 90.805 | 1.00 | 59.82 |
| ATOM | 9252 | CD | GLU | 5159 | 48.350 | 22.630 | 92.014 | 1.00 | 60.02 |
| ATOM | 9253 | OE1 | GLU | 5159 | 47.648 | 21.603 | 92.160 | 1.00 | 58.95 |
| ATOM | 9254 | OE2 | GLU | 5159 | 48.356 | 23.588 | 92.821 | 1.00 | 61.02 |
| ATOM | 9255 | C | GLU | 5159 | 47.045 | 21.793 | 87.769 | 1.00 | 58.78 |
| ATOM | 9256 | O | GLU | 5159 | 47.365 | 22.061 | 86.617 | 1.00 | 59.46 |
| ATOM | 9257 | N | LYS | 5160 | 46.313 | 20.734 | 88.090 | 1.00 | 58.35 |
| ATOM | 9258 | CA | LYS | 5160 | 45.831 | 19.780 | 87.099 | 1.00 | 58.26 |
| ATOM | 9259 | CB | LYS | 5160 | 45.506 | 18.460 | 87.807 | 1.00 | 58.87 |
| ATOM | 9260 | CG | LYS | 5160 | 46.510 | 18.077 | 88.886 | 1.00 | 59.55 |
| ATOM | 9261 | CD | LYS | 5160 | 46.142 | 16.769 | 89.584 | 1.00 | 59.69 |
| ATOM | 9262 | CE | LYS | 5160 | 47.166 | 16.435 | 90.674 | 1.00 | 60.79 |
| ATOM | 9263 | NZ | LYS | 5160 | 46.815 | 15.217 | 91.462 | 1.00 | 61.04 |
| ATOM | 9264 | C | LYS | 5160 | 44.582 | 20.248 | 86.340 | 1.00 | 57.60 |
| ATOM | 9265 | O | LYS | 5160 | 43.939 | 19.449 | 85.651 | 1.00 | 57.47 |
| ATOM | 9266 | N | MSE | 5161 | 44.230 | 21.522 | 86.483 | 1.00 | 56.25 |
| ATOM | 9267 | CA | MSE | 5161 | 43.052 | 22.069 | 85.819 | 1.00 | 55.13 |
| ATOM | 9268 | CB | MSE | 5161 | 42.078 | 22.640 | 86.855 | 1.00 | 54.12 |
| ATOM | 9269 | CG | MSE | 5161 | 41.511 | 21.605 | 87.821 | 1.00 | 52.24 |
| ATOM | 9270 | SE | MSE | 5161 | 40.454 | 22.333 | 89.096 | 1.00 | 50.14 |
| ATOM | 9271 | CE | MSE | 5161 | 39.036 | 22.783 | 88.147 | 1.00 | 50.26 |
| ATOM | 9272 | C | MSE | 5161 | 43.481 | 23.157 | 84.846 | 1.00 | 55.17 |
| ATOM | 9273 | O | MSE | 5161 | 42.667 | 23.958 | 84.378 | 1.00 | 54.94 |
| ATOM | 9274 | N | GLU | 5162 | 44.778 | 23.164 | 84.552 | 1.00 | 55.25 |
| ATOM | 9275 | CA | GLU | 5162 | 45.392 | 24.124 | 83.642 | 1.00 | 55.36 |
| ATOM | 9276 | CB | GLU | 5162 | 46.895 | 23.867 | 83.571 | 1.00 | 57.30 |
| ATOM | 9277 | CG | GLU | 5162 | 47.702 | 24.426 | 84.721 | 1.00 | 59.83 |
| ATOM | 9278 | CD | GLU | 5162 | 47.766 | 25.938 | 84.682 | 1.00 | 61.57 |
| ATOM | 9279 | OE1 | GLU | 5162 | 46.777 | 26.588 | 85.090 | 1.00 | 62.60 |
| ATOM | 9280 | OE2 | GLU | 5162 | 48.803 | 26.475 | 84.228 | 1.00 | 62.35 |
| ATOM | 9281 | C | GLU | 5162 | 44.821 | 24.062 | 82.231 | 1.00 | 54.54 |
| ATOM | 9282 | O | GLU | 5162 | 44.301 | 25.050 | 81.712 | 1.00 | 54.64 |
| ATOM | 9283 | N | LYS | 5163 | 44.944 | 22.886 | 81.616 | 1.00 | 53.09 |
| ATOM | 9284 | CA | LYS | 5163 | 44.489 | 22.636 | 80.248 | 1.00 | 51.49 |
| ATOM | 9285 | CB | LYS | 5163 | 44.886 | 21.212 | 79.840 | 1.00 | 52.08 |
| ATOM | 9286 | CG | LYS | 5163 | 44.658 | 20.856 | 78.375 | 1.00 | 52.74 |
| ATOM | 9287 | CD | LYS | 5163 | 45.028 | 19.390 | 78.116 | 1.00 | 53.08 |
| ATOM | 9288 | CE | LYS | 5163 | 44.801 | 19.002 | 76.662 | 1.00 | 54.30 |
| ATOM | 9289 | NZ | LYS | 5163 | 45.029 | 17.545 | 76.390 | 1.00 | 54.51 |
| ATOM | 9290 | C | LYS | 5163 | 42.994 | 22.814 | 80.080 | 1.00 | 49.87 |
| ATOM | 9291 | O | LYS | 5163 | 42.214 | 21.950 | 80.461 | 1.00 | 50.15 |
| ATOM | 9292 | N | LYS | 5164 | 42.597 | 23.936 | 79.491 | 1.00 | 48.29 |
| ATOM | 9293 | CA | LYS | 5164 | 41.182 | 24.220 | 79.289 | 1.00 | 47.05 |
| ATOM | 9294 | CB | LYS | 5164 | 40.936 | 25.727 | 79.263 | 1.00 | 48.07 |
| ATOM | 9295 | CG | LYS | 5164 | 39.476 | 26.065 | 79.079 | 1.00 | 51.17 |
| ATOM | 9296 | CD | LYS | 5164 | 39.203 | 27.544 | 79.172 | 1.00 | 53.22 |
| ATOM | 9297 | CE | LYS | 5164 | 37.716 | 27.802 | 79.016 | 1.00 | 54.09 |
| ATOM | 9298 | NZ | LYS | 5164 | 37.417 | 29.258 | 78.847 | 1.00 | 56.43 |
| ATOM | 9299 | C | LYS | 5164 | 40.617 | 23.602 | 78.011 | 1.00 | 45.48 |
| ATOM | 9300 | O | LYS | 5164 | 39.503 | 23.083 | 78.007 | 1.00 | 45.08 |
| ATOM | 9301 | N | LEU | 5165 | 41.375 | 23.678 | 76.924 | 1.00 | 43.38 |
| ATOM | 9302 | CA | LEU | 5165 | 40.923 | 23.109 | 75.666 | 1.00 | 41.62 |
| ATOM | 9303 | CB | LEU | 5165 | 41.379 | 23.951 | 74.472 | 1.00 | 41.32 |
| ATOM | 9304 | CG | LEU | 5165 | 41.195 | 23.268 | 73.110 | 1.00 | 41.24 |
| ATOM | 9305 | CD1 | LEU | 5165 | 39.740 | 22.925 | 72.870 | 1.00 | 41.29 |
| ATOM | 9306 | CD2 | LEU | 5165 | 41.703 | 24.168 | 72.011 | 1.00 | 41.18 |
| ATOM | 9307 | C | LEU | 5165 | 41.421 | 21.686 | 75.485 | 1.00 | 40.96 |
| ATOM | 9308 | O | LEU | 5165 | 42.622 | 21.416 | 75.558 | 1.00 | 40.98 |
| ATOM | 9309 | N | HIS | 5166 | 40.488 | 20.776 | 75.253 | 1.00 | 39.24 |
| ATOM | 9310 | CA | HIS | 5166 | 40.840 | 19.392 | 75.047 | 1.00 | 38.05 |
| ATOM | 9311 | CB | HIS | 5166 | 40.090 | 18.502 | 76.029 | 1.00 | 40.32 |
| ATOM | 9312 | CG | HIS | 5166 | 40.919 | 17.381 | 76.558 | 1.00 | 42.46 |
| ATOM | 9313 | CD2 | HIS | 5166 | 41.166 | 16.974 | 77.826 | 1.00 | 43.64 |
| ATOM | 9314 | ND1 | HIS | 5166 | 41.654 | 16.553 | 75.735 | 1.00 | 44.15 |
| ATOM | 9315 | CE1 | HIS | 5166 | 42.320 | 15.684 | 76.474 | 1.00 | 44.44 |
| ATOM | 9316 | NE2 | HIS | 5166 | 42.043 | 15.918 | 77.747 | 1.00 | 45.31 |
| ATOM | 9317 | C | HIS | 5166 | 40.450 | 19.062 | 73.620 | 1.00 | 36.31 |
| ATOM | 9318 | O | HIS | 5166 | 39.263 | 19.005 | 73.280 | 1.00 | 35.77 |
| ATOM | 9319 | N | ALA | 5167 | 41.453 | 18.870 | 72.770 | 1.00 | 34.05 |
| ATOM | 9320 | CA | ALA | 5167 | 41.194 | 18.543 | 71.372 | 1.00 | 31.67 |
| ATOM | 9321 | CB | ALA | 5167 | 41.892 | 19.541 | 70.451 | 1.00 | 31.12 |
| ATOM | 9322 | C | ALA | 5167 | 41.688 | 17.128 | 71.116 | 1.00 | 30.02 |
| ATOM | 9323 | O | ALA | 5167 | 42.823 | 16.782 | 71.457 | 1.00 | 30.28 |
| ATOM | 9324 | N | VAL | 5168 | 40.830 | 16.301 | 70.535 | 1.00 | 27.85 |
| ATOM | 9325 | CA | VAL | 5168 | 41.197 | 14.915 | 70.282 | 1.00 | 26.98 |
| ATOM | 9326 | CB | VAL | 5168 | 40.760 | 13.981 | 71.427 | 1.00 | 26.63 |
| ATOM | 9327 | CG1 | VAL | 5168 | 41.291 | 14.492 | 72.747 | 1.00 | 26.05 |
| ATOM | 9328 | CG2 | VAL | 5168 | 39.234 | 13.865 | 71.450 | 1.00 | 26.09 |
| ATOM | 9329 | C | VAL | 5168 | 40.539 | 14.382 | 69.032 | 1.00 | 26.79 |
| ATOM | 9330 | O | VAL | 5168 | 39.528 | 14.917 | 68.569 | 1.00 | 26.91 |
| ATOM | 9331 | N | PRO | 5169 | 41.110 | 13.317 | 68.455 | 1.00 | 25.85 |
| ATOM | 9332 | CD | PRO | 5169 | 42.369 | 12.637 | 68.772 | 1.00 | 25.90 |
| ATOM | 9333 | CA | PRO | 5169 | 40.515 | 12.742 | 67.253 | 1.00 | 25.82 |
| ATOM | 9334 | CB | PRO | 5169 | 41.566 | 11.751 | 66.769 | 1.00 | 25.30 |
| ATOM | 9335 | CG | PRO | 5169 | 42.812 | 12.204 | 67.411 | 1.00 | 26.70 |
| ATOM | 9336 | C | PRO | 5169 | 39.271 | 11.994 | 67.725 | 1.00 | 25.95 |
| ATOM | 9337 | O | PRO | 5169 | 39.143 | 11.686 | 68.915 | 1.00 | 25.56 |
| ATOM | 9338 | N | ALA | 5170 | 38.362 | 11.704 | 66.807 | 1.00 | 25.17 |
| ATOM | 9339 | CA | ALA | 5170 | 37.154 | 10.957 | 67.150 | 1.00 | 25.99 |
| ATOM | 9340 | CB | ALA | 5170 | 36.235 | 10.822 | 65.934 | 1.00 | 24.37 |
| ATOM | 9341 | C | ALA | 5170 | 37.507 | 9.565 | 67.672 | 1.00 | 26.45 |
| ATOM | 9342 | O | ALA | 5170 | 38.564 | 9.001 | 67.340 | 1.00 | 27.47 |
| ATOM | 9343 | N | ALA | 5171 | 36.620 | 9.030 | 68.503 | 1.00 | 26.49 |
| ATOM | 9344 | CA | ALA | 5171 | 36.773 | 7.703 | 69.099 | 1.00 | 27.73 |
| ATOM | 9345 | CB | ALA | 5171 | 37.268 | 6.693 | 68.051 | 1.00 | 27.30 |
| ATOM | 9346 | C | ALA | 5171 | 37.662 | 7.637 | 70.351 | 1.00 | 28.26 |
| ATOM | 9347 | O | ALA | 5171 | 37.703 | 6.607 | 71.031 | 1.00 | 29.11 |
| ATOM | 9348 | N | LYS | 5172 | 38.380 | 8.711 | 70.659 | 1.00 | 28.06 |
| ATOM | 9349 | CA | LYS | 5172 | 39.210 | 8.729 | 71.867 | 1.00 | 28.45 |
| ATOM | 9350 | CB | LYS | 5172 | 40.168 | 9.923 | 71.872 | 1.00 | 29.22 |
| ATOM | 9351 | CG | LYS | 5172 | 41.542 | 9.646 | 71.302 | 1.00 | 31.73 |
| ATOM | 9352 | CD | LYS | 5172 | 42.610 | 9.696 | 72.391 | 1.00 | 32.76 |
| ATOM | 9353 | CE | LYS | 5172 | 42.800 | 11.114 | 72.901 | 1.00 | 33.23 |
| ATOM | 9354 | NZ | LYS | 5172 | 43.682 | 11.135 | 74.093 | 1.00 | 33.64 |
| ATOM | 9355 | C | LYS | 5172 | 38.344 | 8.848 | 73.115 | 1.00 | 28.61 |
| ATOM | 9356 | O | LYS | 5172 | 37.198 | 9.328 | 73.061 | 1.00 | 28.97 |
| ATOM | 9357 | N | THR | 5173 | 38.898 | 8.414 | 74.240 | 1.00 | 28.02 |
| ATOM | 9358 | CA | THR | 5173 | 38.202 | 8.518 | 75.508 | 1.00 | 28.22 |
| ATOM | 9359 | CB | THR | 5173 | 38.611 | 7.396 | 76.459 | 1.00 | 27.84 |
| ATOM | 9360 | OG1 | THR | 5173 | 38.051 | 6.169 | 75.995 | 1.00 | 29.00 |
| ATOM | 9361 | CG2 | THR | 5173 | 38.102 | 7.670 | 77.867 | 1.00 | 28.16 |
| ATOM | 9362 | C | THR | 5173 | 38.614 | 9.852 | 76.111 | 1.00 | 28.44 |
| ATOM | 9363 | O | THR | 5173 | 39.786 | 10.201 | 76.094 | 1.00 | 28.35 |
| ATOM | 9364 | N | VAL | 5174 | 37.651 | 10.601 | 76.630 | 1.00 | 29.02 |
| ATOM | 9365 | CA | VAL | 5174 | 37.935 | 11.894 | 77.233 | 1.00 | 29.46 |
| ATOM | 9366 | CB | VAL | 5174 | 37.173 | 13.010 | 76.499 | 1.00 | 29.63 |
| ATOM | 9367 | CG1 | VAL | 5174 | 37.406 | 14.350 | 77.186 | 1.00 | 29.47 |
| ATOM | 9368 | CG2 | VAL | 5174 | 37.628 | 13.066 | 75.044 | 1.00 | 29.89 |
| ATOM | 9369 | C | VAL | 5174 | 37.549 | 11.893 | 78.711 | 1.00 | 30.36 |
| ATOM | 9370 | O | VAL | 5174 | 36.501 | 11.381 | 79.102 | 1.00 | 29.66 |
| ATOM | 9371 | N | LYS | 5175 | 38.407 | 12.481 | 79.530 | 1.00 | 31.69 |
| ATOM | 9372 | CA | LYS | 5175 | 38.176 | 12.527 | 80.962 | 1.00 | 32.55 |
| ATOM | 9373 | CB | LYS | 5175 | 39.119 | 11.532 | 81.654 | 1.00 | 33.64 |
| ATOM | 9374 | CG | LYS | 5175 | 38.919 | 11.390 | 83.157 | 1.00 | 36.78 |
| ATOM | 9375 | CD | LYS | 5175 | 40.155 | 10.811 | 83.842 | 1.00 | 38.95 |
| ATOM | 9376 | CE | LYS | 5175 | 41.373 | 11.741 | 83.632 | 1.00 | 42.21 |
| ATOM | 9377 | NZ | LYS | 5175 | 42.633 | 11.362 | 84.376 | 1.00 | 42.57 |
| ATOM | 9378 | C | LYS | 5175 | 38.416 | 13.922 | 81.519 | 1.00 | 32.61 |
| ATOM | 9379 | O | LYS | 5175 | 39.503 | 14.467 | 81.388 | 1.00 | 33.39 |
| ATOM | 9380 | N | PHE | 5176 | 37.395 | 14.502 | 82.136 | 1.00 | 32.69 |
| ATOM | 9381 | CA | PHE | 5176 | 37.527 | 15.817 | 82.747 | 1.00 | 32.63 |
| ATOM | 9382 | CB | PHE | 5176 | 36.444 | 16.769 | 82.251 | 1.00 | 30.55 |
| ATOM | 9383 | CG | PHE | 5176 | 36.569 | 17.127 | 80.808 | 1.00 | 28.69 |
| ATOM | 9384 | CD1 | PHE | 5176 | 37.792 | 17.553 | 80.289 | 1.00 | 28.35 |
| ATOM | 9385 | CD2 | PHE | 5176 | 35.470 | 17.049 | 79.966 | 1.00 | 26.90 |
| ATOM | 9386 | CE1 | PHE | 5176 | 37.919 | 17.898 | 78.946 | 1.00 | 27.95 |
| ATOM | 9387 | CE2 | PHE | 5176 | 35.583 | 17.390 | 78.624 | 1.00 | 27.96 |
| ATOM | 9388 | CZ | PHE | 5176 | 36.816 | 17.818 | 78.109 | 1.00 | 27.38 |
| ATOM | 9389 | C | PHE | 5176 | 37.402 | 15.668 | 84.251 | 1.00 | 34.09 |
| ATOM | 9390 | O | PHE | 5176 | 36.590 | 14.885 | 84.744 | 1.00 | 34.68 |
| ATOM | 9391 | N | LYS | 5177 | 38.203 | 16.415 | 84.994 | 1.00 | 35.92 |
| ATOM | 9392 | CA | LYS | 5177 | 38.124 | 16.332 | 86.443 | 1.00 | 37.62 |
| ATOM | 9393 | CB | LYS | 5177 | 39.158 | 15.351 | 86.985 | 1.00 | 38.74 |
| ATOM | 9394 | CG | LYS | 5177 | 40.576 | 15.740 | 86.711 | 1.00 | 40.69 |
| ATOM | 9395 | CD | LYS | 5177 | 41.383 | 14.512 | 86.343 | 1.00 | 42.75 |
| ATOM | 9396 | CE | LYS | 5177 | 42.517 | 14.867 | 85.378 | 1.00 | 44.42 |
| ATOM | 9397 | NZ | LYS | 5177 | 43.175 | 13.636 | 84.868 | 1.00 | 43.88 |
| ATOM | 9398 | C | LYS | 5177 | 38.277 | 17.669 | 87.137 | 1.00 | 38.24 |
| ATOM | 9399 | O | LYS | 5177 | 38.922 | 18.586 | 86.641 | 1.00 | 38.38 |
| ATOM | 9400 | N | CYS | 5178 | 37.651 | 17.767 | 88.299 | 1.00 | 39.72 |
| ATOM | 9401 | CA | CYS | 5178 | 37.688 | 18.976 | 89.115 | 1.00 | 40.88 |
| ATOM | 9402 | CB | CYS | 5178 | 36.373 | 19.740 | 89.002 | 1.00 | 41.52 |
| ATOM | 9403 | SG | CYS | 5178 | 36.124 | 20.359 | 87.363 | 1.00 | 43.45 |
| ATOM | 9404 | C | CYS | 5178 | 37.898 | 18.579 | 90.556 | 1.00 | 40.80 |
| ATOM | 9405 | O | CYS | 5178 | 37.000 | 18.723 | 91.383 | 1.00 | 40.28 |
| ATOM | 9406 | N | PRO | 5179 | 39.092 | 18.068 | 90.875 | 1.00 | 41.43 |
| ATOM | 9407 | CD | PRO | 5179 | 40.304 | 18.022 | 90.046 | 1.00 | 41.50 |
| ATOM | 9408 | CA | PRO | 5179 | 39.383 | 17.653 | 92.245 | 1.00 | 42.43 |
| ATOM | 9409 | CB | PRO | 5179 | 40.821 | 17.160 | 92.160 | 1.00 | 41.49 |
| ATOM | 9410 | CG | PRO | 5179 | 41.395 | 18.005 | 91.091 | 1.00 | 42.40 |
| ATOM | 9411 | C | PRO | 5179 | 39.197 | 18.805 | 93.220 | 1.00 | 43.71 |
| ATOM | 9412 | O | PRO | 5179 | 39.813 | 19.866 | 93.091 | 1.00 | 44.35 |
| ATOM | 9413 | N | SER | 5180 | 38.309 | 18.602 | 94.179 | 1.00 | 44.77 |
| ATOM | 9414 | CA | SER | 5180 | 38.048 | 19.622 | 95.171 | 1.00 | 46.23 |
| ATOM | 9415 | CB | SER | 5180 | 36.919 | 20.546 | 94.707 | 1.00 | 46.96 |
| ATOM | 9416 | OG | SER | 5180 | 35.795 | 19.820 | 94.254 | 1.00 | 48.19 |
| ATOM | 9417 | C | SER | 5180 | 37.716 | 19.001 | 96.511 | 1.00 | 46.86 |
| ATOM | 9418 | O | SER | 5180 | 37.765 | 17.779 | 96.679 | 1.00 | 46.74 |
| ATOM | 9419 | N | SER | 5181 | 37.384 | 19.855 | 97.469 | 1.00 | 47.71 |
| ATOM | 9420 | CA | SER | 5181 | 37.075 | 19.394 | 98.810 | 1.00 | 48.54 |
| ATOM | 9421 | CB | SER | 5181 | 38.380 | 19.139 | 99.567 | 1.00 | 48.54 |
| ATOM | 9422 | OG | SER | 5181 | 38.133 | 18.606 | 100.851 | 1.00 | 49.12 |
| ATOM | 9423 | C | SER | 5181 | 36.252 | 20.436 | 99.548 | 1.00 | 49.06 |
| ATOM | 9424 | O | SER | 5181 | 35.925 | 21.491 | 99.002 | 1.00 | 48.80 |
| ATOM | 9425 | N | GLY | 5182 | 35.930 | 20.130 | 100.799 | 1.00 | 49.92 |
| ATOM | 9426 | CA | GLY | 5182 | 35.149 | 21.039 | 101.611 | 1.00 | 50.58 |
| ATOM | 9427 | C | GLY | 5182 | 34.309 | 20.234 | 102.576 | 1.00 | 51.41 |
| ATOM | 9428 | O | GLY | 5182 | 34.127 | 19.029 | 102.403 | 1.00 | 51.48 |
| ATOM | 9429 | N | THR | 5183 | 33.788 | 20.900 | 103.596 | 1.00 | 52.15 |
| ATOM | 9430 | CA | THR | 5183 | 32.971 | 20.231 | 104.602 | 1.00 | 52.34 |
| ATOM | 9431 | CB | THR | 5183 | 33.773 | 20.043 | 105.903 | 1.00 | 53.14 |
| ATOM | 9432 | OG1 | THR | 5183 | 34.504 | 21.246 | 106.175 | 1.00 | 54.65 |
| ATOM | 9433 | CG2 | THR | 5183 | 34.750 | 18.879 | 105.773 | 1.00 | 53.07 |
| ATOM | 9434 | C | THR | 5183 | 31.721 | 21.046 | 104.918 | 1.00 | 51.79 |
| ATOM | 9435 | O | THR | 5183 | 31.813 | 22.232 | 105.238 | 1.00 | 51.45 |
| ATOM | 9436 | N | PRO | 5184 | 30.533 | 20.418 | 104.821 | 1.00 | 51.56 |
| ATOM | 9437 | CD | PRO | 5184 | 29.228 | 21.096 | 104.935 | 1.00 | 51.51 |
| ATOM | 9438 | CA | PRO | 5184 | 30.367 | 19.011 | 104.431 | 1.00 | 51.20 |
| ATOM | 9439 | CB | PRO | 5184 | 28.862 | 18.797 | 104.575 | 1.00 | 51.59 |
| ATOM | 9440 | CG | PRO | 5184 | 28.299 | 20.153 | 104.195 | 1.00 | 51.73 |
| ATOM | 9441 | C | PRO | 5184 | 30.864 | 18.748 | 103.002 | 1.00 | 51.20 |
| ATOM | 9442 | O | PRO | 5184 | 31.060 | 19.688 | 102.226 | 1.00 | 51.48 |
| ATOM | 9443 | N | GLN | 5185 | 31.082 | 17.480 | 102.664 | 1.00 | 50.73 |
| ATOM | 9444 | CA | GLN | 5185 | 31.544 | 17.119 | 101.325 | 1.00 | 50.41 |
| ATOM | 9445 | CB | GLN | 5185 | 31.572 | 15.599 | 101.139 | 1.00 | 51.37 |
| ATOM | 9446 | CG | GLN | 5185 | 32.857 | 14.959 | 101.600 | 1.00 | 53.16 |
| ATOM | 9447 | CD | GLN | 5185 | 34.027 | 15.377 | 100.749 | 1.00 | 54.24 |
| ATOM | 9448 | OE1 | GLN | 5185 | 34.063 | 15.094 | 99.549 | 1.00 | 54.60 |
| ATOM | 9449 | NE2 | GLN | 5185 | 34.994 | 16.061 | 101.358 | 1.00 | 54.51 |
| ATOM | 9450 | C | GLN | 5185 | 30.616 | 17.716 | 100.286 | 1.00 | 49.53 |
| ATOM | 9451 | O | GLN | 5185 | 29.402 | 17.511 | 100.339 | 1.00 | 49.75 |
| ATOM | 9452 | N | PRO | 5186 | 31.173 | 18.479 | 99.331 | 1.00 | 48.36 |
| ATOM | 9453 | CD | PRO | 5186 | 32.557 | 18.983 | 99.300 | 1.00 | 47.70 |
| ATOM | 9454 | CA | PRO | 5186 | 30.361 | 19.101 | 98.279 | 1.00 | 47.49 |
| ATOM | 9455 | CB | PRO | 5186 | 31.269 | 20.206 | 97.757 | 1.00 | 47.56 |
| ATOM | 9456 | CG | PRO | 5186 | 32.638 | 19.615 | 97.941 | 1.00 | 47.97 |
| ATOM | 9457 | C | PRO | 5186 | 29.920 | 18.124 | 97.179 | 1.00 | 46.58 |
| ATOM | 9458 | O | PRO | 5186 | 30.589 | 17.126 | 96.906 | 1.00 | 46.38 |
| ATOM | 9459 | N | THR | 5187 | 28.777 | 18.408 | 96.566 | 1.00 | 45.81 |
| ATOM | 9460 | CA | THR | 5187 | 28.257 | 17.564 | 95.497 | 1.00 | 45.17 |
| ATOM | 9461 | CB | THR | 5187 | 26.743 | 17.717 | 95.345 | 1.00 | 45.69 |
| ATOM | 9462 | OG1 | THR | 5187 | 26.419 | 19.111 | 95.208 | 1.00 | 45.30 |
| ATOM | 9463 | CG2 | THR | 5187 | 26.023 | 17.115 | 96.544 | 1.00 | 44.98 |
| ATOM | 9464 | C | THR | 5187 | 28.894 | 17.979 | 94.183 | 1.00 | 44.70 |
| ATOM | 9465 | O | THR | 5187 | 29.455 | 19.070 | 94.065 | 1.00 | 44.83 |
| ATOM | 9466 | N | LEU | 5188 | 28.788 | 17.116 | 93.186 | 1.00 | 44.03 |
| ATOM | 9467 | CA | LEU | 5188 | 29.375 | 17.402 | 91.890 | 1.00 | 42.91 |
| ATOM | 9468 | CB | LEU | 5188 | 30.706 | 16.643 | 91.765 | 1.00 | 43.62 |
| ATOM | 9469 | CG | LEU | 5188 | 31.532 | 16.512 | 90.478 | 1.00 | 43.43 |
| ATOM | 9470 | CD1 | LEU | 5188 | 31.034 | 15.337 | 89.689 | 1.00 | 43.74 |
| ATOM | 9471 | CD2 | LEU | 5188 | 31.486 | 17.791 | 89.669 | 1.00 | 43.48 |
| ATOM | 9472 | C | LEU | 5188 | 28.434 | 17.033 | 90.757 | 1.00 | 42.36 |
| ATOM | 9473 | O | LEU | 5188 | 27.991 | 15.884 | 90.646 | 1.00 | 42.60 |
| ATOM | 9474 | N | ARG | 5189 | 28.099 | 18.023 | 89.937 | 1.00 | 41.15 |
| ATOM | 9475 | CA | ARG | 5189 | 27.256 | 17.773 | 88.782 | 1.00 | 39.57 |
| ATOM | 9476 | CB | ARG | 5189 | 25.816 | 18.241 | 89.020 | 1.00 | 39.71 |
| ATOM | 9477 | CG | ARG | 5189 | 25.565 | 19.721 | 89.194 | 1.00 | 39.79 |
| ATOM | 9478 | CD | ARG | 5189 | 24.065 | 19.895 | 89.489 | 1.00 | 39.62 |
| ATOM | 9479 | NE | ARG | 5189 | 23.508 | 21.120 | 88.928 | 1.00 | 40.24 |
| ATOM | 9480 | CZ | ARG | 5189 | 23.718 | 22.332 | 89.429 | 1.00 | 41.01 |
| ATOM | 9481 | NH1 | ARG | 5189 | 24.477 | 22.476 | 90.513 | 1.00 | 41.24 |
| ATOM | 9482 | NH2 | ARG | 5189 | 23.167 | 23.398 | 88.849 | 1.00 | 40.49 |
| ATOM | 9483 | C | ARG | 5189 | 27.885 | 18.445 | 87.559 | 1.00 | 38.45 |
| ATOM | 9484 | O | ARG | 5189 | 28.615 | 19.437 | 87.680 | 1.00 | 37.05 |
| ATOM | 9485 | N | TRP | 5190 | 27.627 | 17.876 | 86.385 | 1.00 | 36.80 |
| ATOM | 9486 | CA | TRP | 5190 | 28.195 | 18.402 | 85.166 | 1.00 | 34.85 |
| ATOM | 9487 | CB | TRP | 5190 | 28.998 | 17.318 | 84.458 | 1.00 | 34.46 |
| ATOM | 9488 | CG | TRP | 5190 | 30.226 | 16.850 | 85.199 | 1.00 | 32.88 |
| ATOM | 9489 | CD2 | TRP | 5190 | 31.565 | 17.330 | 85.019 | 1.00 | 31.94 |
| ATOM | 9490 | CE2 | TRP | 5190 | 32.395 | 16.591 | 85.896 | 1.00 | 31.88 |
| ATOM | 9491 | CE3 | TRP | 5190 | 32.143 | 18.309 | 84.199 | 1.00 | 31.30 |
| ATOM | 9492 | CD1 | TRP | 5190 | 30.295 | 15.871 | 86.153 | 1.00 | 32.61 |
| ATOM | 9493 | NE1 | TRP | 5190 | 31.598 | 15.708 | 86.574 | 1.00 | 32.37 |
| ATOM | 9494 | CZ2 | TRP | 5190 | 33.776 | 16.806 | 85.975 | 1.00 | 31.55 |
| ATOM | 9495 | CZ3 | TRP | 5190 | 33.519 | 18.521 | 84.276 | 1.00 | 29.99 |
| ATOM | 9496 | CH2 | TRP | 5190 | 34.317 | 17.774 | 85.159 | 1.00 | 30.87 |
| ATOM | 9497 | C | TRP | 5190 | 27.166 | 18.957 | 84.210 | 1.00 | 34.86 |
| ATOM | 9498 | O | TRP | 5190 | 26.036 | 18.481 | 84.139 | 1.00 | 35.73 |
| ATOM | 9499 | N | LEU | 5191 | 27.573 | 19.974 | 83.463 | 1.00 | 34.56 |
| ATOM | 9500 | CA | LEU | 5191 | 26.704 | 20.597 | 82.478 | 1.00 | 34.59 |
| ATOM | 9501 | CB | LEU | 5191 | 26.460 | 22.067 | 82.837 | 1.00 | 34.71 |
| ATOM | 9502 | CG | LEU | 5191 | 25.847 | 22.414 | 84.199 | 1.00 | 34.90 |
| ATOM | 9503 | CD1 | LEU | 5191 | 25.948 | 23.911 | 84.419 | 1.00 | 34.59 |
| ATOM | 9504 | CD2 | LEU | 5191 | 24.400 | 21.955 | 84.268 | 1.00 | 33.97 |
| ATOM | 9505 | C | LEU | 5191 | 27.367 | 20.529 | 81.106 | 1.00 | 34.32 |
| ATOM | 9506 | O | LEU | 5191 | 28.587 | 20.514 | 80.996 | 1.00 | 33.61 |
| ATOM | 9507 | N | LYS | 5192 | 26.553 | 20.483 | 80.063 | 1.00 | 34.61 |
| ATOM | 9508 | CA | LYS | 5192 | 27.049 | 20.460 | 78.698 | 1.00 | 35.08 |
| ATOM | 9509 | CB | LYS | 5192 | 26.543 | 19.207 | 77.974 | 1.00 | 35.25 |
| ATOM | 9510 | CG | LYS | 5192 | 26.711 | 19.222 | 76.455 | 1.00 | 34.36 |
| ATOM | 9511 | CD | LYS | 5192 | 27.488 | 18.009 | 75.994 | 1.00 | 34.05 |
| ATOM | 9512 | CE | LYS | 5192 | 27.512 | 17.875 | 74.473 | 1.00 | 33.06 |
| ATOM | 9513 | NZ | LYS | 5192 | 26.219 | 17.386 | 73.952 | 1.00 | 32.26 |
| ATOM | 9514 | C | LYS | 5192 | 26.487 | 21.728 | 78.061 | 1.00 | 35.53 |
| ATOM | 9515 | O | LYS | 5192 | 25.279 | 21.855 | 77.859 | 1.00 | 34.88 |
| ATOM | 9516 | N | ASN | 5193 | 27.368 | 22.674 | 77.768 | 1.00 | 36.56 |
| ATOM | 9517 | CA | ASN | 5193 | 26.970 | 23.949 | 77.191 | 1.00 | 37.26 |
| ATOM | 9518 | CB | ASN | 5193 | 26.424 | 23.747 | 75.782 | 1.00 | 37.99 |
| ATOM | 9519 | CG | ASN | 5193 | 27.463 | 23.180 | 74.855 | 1.00 | 38.44 |
| ATOM | 9520 | OD1 | ASN | 5193 | 28.636 | 23.561 | 74.927 | 1.00 | 38.95 |
| ATOM | 9521 | ND2 | ASN | 5193 | 27.050 | 22.272 | 73.974 | 1.00 | 38.13 |
| ATOM | 9522 | C | ASN | 5193 | 25.956 | 24.676 | 78.064 | 1.00 | 37.84 |
| ATOM | 9523 | O | ASN | 5193 | 24.969 | 25.231 | 77.569 | 1.00 | 38.66 |
| ATOM | 9524 | N | GLY | 5194 | 26.213 | 24.662 | 79.369 | 1.00 | 37.72 |
| ATOM | 9525 | CA | GLY | 5194 | 25.346 | 25.342 | 80.308 | 1.00 | 38.71 |
| ATOM | 9526 | C | GLY | 5194 | 24.060 | 24.652 | 80.714 | 1.00 | 39.68 |
| ATOM | 9527 | O | GLY | 5194 | 23.416 | 25.079 | 81.671 | 1.00 | 40.31 |
| ATOM | 9528 | N | LYS | 5195 | 23.668 | 23.605 | 80.000 | 1.00 | 40.23 |
| ATOM | 9529 | CA | LYS | 5195 | 22.446 | 22.897 | 80.350 | 1.00 | 41.75 |
| ATOM | 9530 | CB | LYS | 5195 | 21.627 | 22.578 | 79.100 | 1.00 | 43.35 |
| ATOM | 9531 | CG | LYS | 5195 | 21.058 | 23.815 | 78.448 | 1.00 | 45.41 |
| ATOM | 9532 | CD | LYS | 5195 | 19.878 | 23.487 | 77.560 | 1.00 | 47.18 |
| ATOM | 9533 | CE | LYS | 5195 | 19.229 | 24.772 | 77.059 | 1.00 | 48.84 |
| ATOM | 9534 | NZ | LYS | 5195 | 20.237 | 25.692 | 76.428 | 1.00 | 49.22 |
| ATOM | 9535 | C | LYS | 5195 | 22.705 | 21.616 | 81.130 | 1.00 | 42.05 |
| ATOM | 9536 | O | LYS | 5195 | 23.853 | 21.204 | 81.316 | 1.00 | 41.86 |
| ATOM | 9537 | N | GLU | 5196 | 21.632 | 20.992 | 81.601 | 1.00 | 42.29 |
| ATOM | 9538 | CA | GLU | 5196 | 21.771 | 19.760 | 82.353 | 1.00 | 42.98 |
| ATOM | 9539 | CB | GLU | 5196 | 20.426 | 19.341 | 82.943 | 1.00 | 45.07 |
| ATOM | 9540 | CG | GLU | 5196 | 20.538 | 18.212 | 83.963 | 1.00 | 47.86 |
| ATOM | 9541 | CD | GLU | 5196 | 19.194 | 17.800 | 84.549 | 1.00 | 49.50 |
| ATOM | 9542 | OE1 | GLU | 5196 | 19.187 | 16.920 | 85.448 | 1.00 | 49.96 |
| ATOM | 9543 | OE2 | GLU | 5196 | 18.154 | 18.351 | 84.108 | 1.00 | 50.22 |
| ATOM | 9544 | C | GLU | 5196 | 22.288 | 18.675 | 81.416 | 1.00 | 42.25 |
| ATOM | 9545 | O | GLU | 5196 | 21.910 | 18.623 | 80.247 | 1.00 | 42.56 |
| ATOM | 9546 | N | PHE | 5197 | 23.159 | 17.815 | 81.926 | 1.00 | 41.43 |
| ATOM | 9547 | CA | PHE | 5197 | 23.712 | 16.742 | 81.114 | 1.00 | 40.97 |
| ATOM | 9548 | CB | PHE | 5197 | 25.237 | 16.895 | 81.011 | 1.00 | 39.97 |
| ATOM | 9549 | CG | PHE | 5197 | 25.876 | 16.024 | 79.959 | 1.00 | 38.60 |
| ATOM | 9550 | CD1 | PHE | 5197 | 25.252 | 15.813 | 78.730 | 1.00 | 38.13 |
| ATOM | 9551 | CD2 | PHE | 5197 | 27.126 | 15.452 | 80.180 | 1.00 | 38.30 |
| ATOM | 9552 | CE1 | PHE | 5197 | 25.860 | 15.047 | 77.741 | 1.00 | 36.86 |
| ATOM | 9553 | CE2 | PHE | 5197 | 27.745 | 14.683 | 79.192 | 1.00 | 38.21 |
| ATOM | 9554 | CZ | PHE | 5197 | 27.110 | 14.482 | 77.974 | 1.00 | 37.53 |
| ATOM | 9555 | C | PHE | 5197 | 23.339 | 15.385 | 81.703 | 1.00 | 41.06 |
| ATOM | 9556 | O | PHE | 5197 | 23.682 | 15.068 | 82.839 | 1.00 | 40.43 |
| ATOM | 9557 | N | LYS | 5198 | 22.606 | 14.600 | 80.929 | 1.00 | 41.64 |
| ATOM | 9558 | CA | LYS | 5198 | 22.191 | 13.278 | 81.368 | 1.00 | 41.76 |
| ATOM | 9559 | CB | LYS | 5198 | 20.669 | 13.157 | 81.321 | 1.00 | 43.01 |
| ATOM | 9560 | CG | LYS | 5198 | 19.945 | 14.133 | 82.246 | 1.00 | 44.73 |
| ATOM | 9561 | CD | LYS | 5198 | 18.431 | 13.996 | 82.101 | 1.00 | 46.42 |
| ATOM | 9562 | CE | LYS | 5198 | 17.698 | 14.824 | 83.149 | 1.00 | 47.27 |
| ATOM | 9563 | NZ | LYS | 5198 | 16.216 | 14.653 | 83.058 | 1.00 | 47.56 |
| ATOM | 9564 | C | LYS | 5198 | 22.816 | 12.215 | 80.485 | 1.00 | 41.34 |
| ATOM | 9565 | O | LYS | 5198 | 22.965 | 12.401 | 79.273 | 1.00 | 41.14 |
| ATOM | 9566 | N | PRO | 5199 | 23.208 | 11.089 | 81.086 | 1.00 | 40.95 |
| ATOM | 9567 | CD | PRO | 5199 | 23.194 | 10.791 | 82.523 | 1.00 | 40.54 |
| ATOM | 9568 | CA | PRO | 5199 | 23.821 | 9.988 | 80.341 | 1.00 | 40.68 |
| ATOM | 9569 | CB | PRO | 5199 | 23.856 | 8.847 | 81.360 | 1.00 | 41.12 |
| ATOM | 9570 | CG | PRO | 5199 | 22.953 | 9.323 | 82.505 | 1.00 | 40.85 |
| ATOM | 9571 | C | PRO | 5199 | 23.092 | 9.621 | 79.056 | 1.00 | 40.43 |
| ATOM | 9572 | O | PRO | 5199 | 23.722 | 9.227 | 78.084 | 1.00 | 41.10 |
| ATOM | 9573 | N | ASP | 5200 | 21.774 | 9.765 | 79.031 | 1.00 | 40.57 |
| ATOM | 9574 | CA | ASP | 5200 | 21.021 | 9.431 | 77.822 | 1.00 | 41.32 |
| ATOM | 9575 | CB | ASP | 5200 | 19.523 | 9.348 | 78.109 | 1.00 | 43.16 |
| ATOM | 9576 | CG | ASP | 5200 | 19.143 | 8.119 | 78.907 | 1.00 | 45.37 |
| ATOM | 9577 | OD1 | ASP | 5200 | 18.487 | 8.303 | 79.957 | 1.00 | 46.96 |
| ATOM | 9578 | OD2 | ASP | 5200 | 19.490 | 6.982 | 78.492 | 1.00 | 45.44 |
| ATOM | 9579 | C | ASP | 5200 | 21.230 | 10.432 | 76.698 | 1.00 | 40.91 |
| ATOM | 9580 | O | ASP | 5200 | 20.683 | 10.266 | 75.602 | 1.00 | 40.29 |
| ATOM | 9581 | N | HIS | 5201 | 22.003 | 11.479 | 76.968 | 1.00 | 40.30 |
| ATOM | 9582 | CA | HIS | 5201 | 22.264 | 12.496 | 75.958 | 1.00 | 39.47 |
| ATOM | 9583 | CB | HIS | 5201 | 22.786 | 13.788 | 76.597 | 1.00 | 40.09 |
| ATOM | 9584 | CG | HIS | 5201 | 21.761 | 14.533 | 77.394 | 1.00 | 40.40 |
| ATOM | 9585 | CD2 | HIS | 5201 | 21.895 | 15.498 | 78.335 | 1.00 | 40.24 |
| ATOM | 9586 | ND1 | HIS | 5201 | 20.406 | 14.339 | 77.235 | 1.00 | 40.89 |
| ATOM | 9587 | CE1 | HIS | 5201 | 19.748 | 15.149 | 78.045 | 1.00 | 40.51 |
| ATOM | 9588 | NE2 | HIS | 5201 | 20.629 | 15.863 | 78.724 | 1.00 | 41.32 |
| ATOM | 9589 | C | HIS | 5201 | 23.267 | 12.028 | 74.914 | 1.00 | 38.37 |
| ATOM | 9590 | O | HIS | 5201 | 23.441 | 12.680 | 73.892 | 1.00 | 38.58 |
| ATOM | 9591 | N | ARG | 5202 | 23.941 | 10.913 | 75.172 | 1.00 | 37.04 |
| ATOM | 9592 | CA | ARG | 5202 | 24.901 | 10.398 | 74.207 | 1.00 | 36.25 |
| ATOM | 9593 | CB | ARG | 5202 | 26.316 | 10.922 | 74.527 | 1.00 | 34.96 |
| ATOM | 9594 | CG | ARG | 5202 | 26.984 | 10.323 | 75.761 | 1.00 | 33.46 |
| ATOM | 9595 | CD | ARG | 5202 | 28.284 | 11.056 | 76.141 | 1.00 | 31.99 |
| ATOM | 9596 | NE | ARG | 5202 | 29.298 | 11.059 | 75.090 | 1.00 | 30.02 |
| ATOM | 9597 | CZ | ARG | 5202 | 30.122 | 10.050 | 74.823 | 1.00 | 29.98 |
| ATOM | 9598 | NH1 | ARG | 5202 | 30.066 | 8.933 | 75.540 | 1.00 | 30.02 |
| ATOM | 9599 | NH2 | ARG | 5202 | 30.993 | 10.149 | 73.820 | 1.00 | 27.77 |
| ATOM | 9600 | C | ARG | 5202 | 24.871 | 8.879 | 74.222 | 1.00 | 36.67 |
| ATOM | 9601 | O | ARG | 5202 | 24.640 | 8.273 | 75.264 | 1.00 | 37.17 |
| ATOM | 9602 | N | ILE | 5203 | 25.074 | 8.266 | 73.062 | 1.00 | 37.06 |
| ATOM | 9603 | CA | ILE | 5203 | 25.083 | 6.816 | 72.977 | 1.00 | 38.70 |
| ATOM | 9604 | CB | ILE | 5203 | 25.327 | 6.355 | 71.531 | 1.00 | 39.18 |
| ATOM | 9605 | CG2 | ILE | 5203 | 24.060 | 6.559 | 70.712 | 1.00 | 39.02 |
| ATOM | 9606 | CG1 | ILE | 5203 | 26.521 | 7.129 | 70.945 | 1.00 | 40.64 |
| ATOM | 9607 | CD1 | ILE | 5203 | 26.996 | 6.643 | 69.563 | 1.00 | 41.41 |
| ATOM | 9608 | C | ILE | 5203 | 26.205 | 6.290 | 73.874 | 1.00 | 39.36 |
| ATOM | 9609 | O | ILE | 5203 | 27.359 | 6.730 | 73.773 | 1.00 | 38.86 |
| ATOM | 9610 | N | GLY | 5204 | 25.857 | 5.360 | 74.760 | 1.00 | 39.58 |
| ATOM | 9611 | CA | GLY | 5204 | 26.846 | 4.809 | 75.663 | 1.00 | 39.81 |
| ATOM | 9612 | C | GLY | 5204 | 26.931 | 5.612 | 76.941 | 1.00 | 39.53 |
| ATOM | 9613 | O | GLY | 5204 | 27.554 | 5.183 | 77.907 | 1.00 | 40.63 |
| ATOM | 9614 | N | GLY | 5205 | 26.309 | 6.786 | 76.943 | 1.00 | 39.01 |
| ATOM | 9615 | CA | GLY | 5205 | 26.322 | 7.632 | 78.124 | 1.00 | 38.72 |
| ATOM | 9616 | C | GLY | 5205 | 27.715 | 8.001 | 78.583 | 1.00 | 38.43 |
| ATOM | 9617 | O | GLY | 5205 | 28.653 | 8.032 | 77.791 | 1.00 | 38.58 |
| ATOM | 9618 | N | TYR | 5206 | 27.849 | 8.284 | 79.869 | 1.00 | 38.68 |
| ATOM | 9619 | CA | TYR | 5206 | 29.127 | 8.655 | 80.444 | 1.00 | 39.09 |
| ATOM | 9620 | CB | TYR | 5206 | 29.319 | 10.162 | 80.342 | 1.00 | 38.47 |
| ATOM | 9621 | CG | TYR | 5206 | 28.275 | 10.950 | 81.077 | 1.00 | 39.10 |
| ATOM | 9622 | CD1 | TYR | 5206 | 28.337 | 11.106 | 82.458 | 1.00 | 39.20 |
| ATOM | 9623 | CE1 | TYR | 5206 | 27.371 | 11.839 | 83.146 | 1.00 | 39.80 |
| ATOM | 9624 | CD2 | TYR | 5206 | 27.213 | 11.548 | 80.395 | 1.00 | 39.86 |
| ATOM | 9625 | CE2 | TYR | 5206 | 26.243 | 12.282 | 81.073 | 1.00 | 39.98 |
| ATOM | 9626 | CZ | TYR | 5206 | 26.330 | 12.423 | 82.447 | 1.00 | 40.02 |
| ATOM | 9627 | OH | TYR | 5206 | 25.380 | 13.150 | 83.126 | 1.00 | 40.77 |
| ATOM | 9628 | C | TYR | 5206 | 29.191 | 8.195 | 81.901 | 1.00 | 39.81 |
| ATOM | 9629 | O | TYR | 5206 | 28.188 | 7.781 | 82.478 | 1.00 | 39.76 |
| ATOM | 9630 | N | LYS | 5207 | 30.376 | 8.270 | 82.493 | 1.00 | 40.54 |
| ATOM | 9631 | CA | LYS | 5207 | 30.579 | 7.813 | 83.852 | 1.00 | 41.86 |
| ATOM | 9632 | CB | LYS | 5207 | 31.600 | 6.677 | 83.863 | 1.00 | 42.70 |
| ATOM | 9633 | CG | LYS | 5207 | 31.099 | 5.369 | 83.308 | 1.00 | 44.72 |
| ATOM | 9634 | CD | LYS | 5207 | 30.358 | 4.608 | 84.378 | 1.00 | 45.94 |
| ATOM | 9635 | CE | LYS | 5207 | 30.673 | 3.128 | 84.295 | 1.00 | 47.00 |
| ATOM | 9636 | NZ | LYS | 5207 | 30.186 | 2.416 | 85.512 | 1.00 | 47.67 |
| ATOM | 9637 | C | LYS | 5207 | 31.103 | 8.906 | 84.745 | 1.00 | 41.86 |
| ATOM | 9638 | O | LYS | 5207 | 32.077 | 9.558 | 84.401 | 1.00 | 42.79 |
| ATOM | 9639 | N | VAL | 5208 | 30.480 | 9.096 | 85.902 | 1.00 | 42.21 |
| ATOM | 9640 | CA | VAL | 5208 | 30.950 | 10.091 | 86.847 | 1.00 | 42.27 |
| ATOM | 9641 | CB | VAL | 5208 | 29.834 | 11.040 | 87.270 | 1.00 | 42.10 |
| ATOM | 9642 | CG1 | VAL | 5208 | 30.334 | 11.964 | 88.368 | 1.00 | 41.73 |
| ATOM | 9643 | CG2 | VAL | 5208 | 29.368 | 11.844 | 86.080 | 1.00 | 41.58 |
| ATOM | 9644 | C | VAL | 5208 | 31.476 | 9.377 | 88.074 | 1.00 | 42.67 |
| ATOM | 9645 | O | VAL | 5208 | 30.707 | 8.764 | 88.803 | 1.00 | 42.91 |
| ATOM | 9646 | N | ARG | 5209 | 32.787 | 9.422 | 88.287 | 1.00 | 43.66 |
| ATOM | 9647 | CA | ARG | 5209 | 33.357 | 8.791 | 89.470 | 1.00 | 44.18 |
| ATOM | 9648 | CB | ARG | 5209 | 34.670 | 8.087 | 89.154 | 1.00 | 46.05 |
| ATOM | 9649 | CG | ARG | 5209 | 35.055 | 7.141 | 90.277 | 1.00 | 49.41 |
| ATOM | 9650 | CD | ARG | 5209 | 36.445 | 6.550 | 90.129 | 1.00 | 51.70 |
| ATOM | 9651 | NE | ARG | 5209 | 36.775 | 5.738 | 91.299 | 1.00 | 53.42 |
| ATOM | 9652 | CZ | ARG | 5209 | 37.913 | 5.066 | 91.453 | 1.00 | 54.40 |
| ATOM | 9653 | NH1 | ARG | 5209 | 38.849 | 5.101 | 90.505 | 1.00 | 54.90 |
| ATOM | 9654 | NH2 | ARG | 5209 | 38.118 | 4.360 | 92.559 | 1.00 | 54.33 |
| ATOM | 9655 | C | ARG | 5209 | 33.589 | 9.923 | 90.457 | 1.00 | 43.66 |
| ATOM | 9656 | O | ARG | 5209 | 34.512 | 10.725 | 90.292 | 1.00 | 43.48 |
| ATOM | 9657 | N | TYR | 5210 | 32.741 | 9.985 | 91.478 | 1.00 | 42.71 |
| ATOM | 9658 | CA | TYR | 5210 | 32.826 | 11.056 | 92.453 | 1.00 | 41.92 |
| ATOM | 9659 | CB | TYR | 5210 | 31.567 | 11.066 | 93.320 | 1.00 | 40.59 |
| ATOM | 9660 | CG | TYR | 5210 | 30.321 | 11.398 | 92.514 | 1.00 | 39.29 |
| ATOM | 9661 | CD1 | TYR | 5210 | 29.601 | 10.400 | 91.842 | 1.00 | 38.84 |
| ATOM | 9662 | CE1 | TYR | 5210 | 28.473 | 10.710 | 91.068 | 1.00 | 37.89 |
| ATOM | 9663 | CD2 | TYR | 5210 | 29.889 | 12.712 | 92.391 | 1.00 | 38.87 |
| ATOM | 9664 | CE2 | TYR | 5210 | 28.770 | 13.038 | 91.618 | 1.00 | 39.02 |
| ATOM | 9665 | CZ | TYR | 5210 | 28.063 | 12.036 | 90.960 | 1.00 | 38.69 |
| ATOM | 9666 | OH | TYR | 5210 | 26.953 | 12.381 | 90.212 | 1.00 | 37.37 |
| ATOM | 9667 | C | TYR | 5210 | 34.088 | 11.051 | 93.294 | 1.00 | 41.80 |
| ATOM | 9668 | O | TYR | 5210 | 34.559 | 12.105 | 93.715 | 1.00 | 42.31 |
| ATOM | 9669 | N | ALA | 5211 | 34.656 | 9.873 | 93.505 | 1.00 | 41.25 |
| ATOM | 9670 | CA | ALA | 5211 | 35.876 | 9.762 | 94.289 | 1.00 | 41.02 |
| ATOM | 9671 | CB | ALA | 5211 | 36.284 | 8.279 | 94.417 | 1.00 | 40.83 |
| ATOM | 9672 | C | ALA | 5211 | 37.006 | 10.567 | 93.639 | 1.00 | 40.99 |
| ATOM | 9673 | O | ALA | 5211 | 37.860 | 11.125 | 94.329 | 1.00 | 41.22 |
| ATOM | 9674 | N | THR | 5212 | 37.007 | 10.618 | 92.311 | 1.00 | 40.25 |
| ATOM | 9675 | CA | THR | 5212 | 38.030 | 11.343 | 91.579 | 1.00 | 39.35 |
| ATOM | 9676 | CB | THR | 5212 | 38.651 | 10.460 | 90.482 | 1.00 | 39.17 |
| ATOM | 9677 | OG1 | THR | 5212 | 37.609 | 9.818 | 89.735 | 1.00 | 39.57 |
| ATOM | 9678 | CG2 | THR | 5212 | 39.551 | 9.414 | 91.095 | 1.00 | 38.77 |
| ATOM | 9679 | C | THR | 5212 | 37.503 | 12.630 | 90.943 | 1.00 | 39.23 |
| ATOM | 9680 | O | THR | 5212 | 38.187 | 13.258 | 90.127 | 1.00 | 39.96 |
| ATOM | 9681 | N | TRP | 5213 | 36.289 | 13.027 | 91.309 | 1.00 | 38.45 |
| ATOM | 9682 | CA | TRP | 5213 | 35.711 | 14.254 | 90.768 | 1.00 | 38.05 |
| ATOM | 9683 | CB | TRP | 5213 | 36.420 | 15.454 | 91.377 | 1.00 | 38.33 |
| ATOM | 9684 | CG | TRP | 5213 | 36.424 | 15.389 | 92.844 | 1.00 | 39.08 |
| ATOM | 9685 | CD2 | TRP | 5213 | 35.425 | 15.924 | 93.717 | 1.00 | 39.01 |
| ATOM | 9686 | CE2 | TRP | 5213 | 35.764 | 15.528 | 95.027 | 1.00 | 39.02 |
| ATOM | 9687 | CE3 | TRP | 5213 | 34.273 | 16.699 | 93.517 | 1.00 | 38.76 |
| ATOM | 9688 | CD1 | TRP | 5213 | 37.310 | 14.712 | 93.636 | 1.00 | 38.45 |
| ATOM | 9689 | NE1 | TRP | 5213 | 36.917 | 14.790 | 94.947 | 1.00 | 38.46 |
| ATOM | 9690 | CZ2 | TRP | 5213 | 34.987 | 15.882 | 96.139 | 1.00 | 40.02 |
| ATOM | 9691 | CZ3 | TRP | 5213 | 33.502 | 17.053 | 94.617 | 1.00 | 38.88 |
| ATOM | 9692 | CH2 | TRP | 5213 | 33.862 | 16.644 | 95.913 | 1.00 | 39.28 |
| ATOM | 9693 | C | TRP | 5213 | 35.864 | 14.302 | 89.258 | 1.00 | 37.71 |
| ATOM | 9694 | O | TRP | 5213 | 36.275 | 15.320 | 88.701 | 1.00 | 37.23 |
| ATOM | 9695 | N | SER | 5214 | 35.530 | 13.203 | 88.596 | 1.00 | 37.39 |
| ATOM | 9696 | CA | SER | 5214 | 35.683 | 13.150 | 87.160 | 1.00 | 36.89 |
| ATOM | 9697 | CB | SER | 5214 | 36.911 | 12.321 | 86.826 | 1.00 | 37.30 |
| ATOM | 9698 | OG | SER | 5214 | 36.828 | 11.070 | 87.467 | 1.00 | 39.11 |
| ATOM | 9699 | C | SER | 5214 | 34.491 | 12.611 | 86.404 | 1.00 | 36.13 |
| ATOM | 9700 | O | SER | 5214 | 33.610 | 11.958 | 86.968 | 1.00 | 36.69 |
| ATOM | 9701 | N | ILE | 5215 | 34.476 | 12.920 | 85.113 | 1.00 | 34.72 |
| ATOM | 9702 | CA | ILE | 5215 | 33.440 | 12.465 | 84.207 | 1.00 | 33.30 |
| ATOM | 9703 | CB | ILE | 5215 | 32.576 | 13.622 | 83.679 | 1.00 | 32.59 |
| ATOM | 9704 | CG2 | ILE | 5215 | 33.455 | 14.699 | 83.080 | 1.00 | 31.09 |
| ATOM | 9705 | CG1 | ILE | 5215 | 31.573 | 13.079 | 82.654 | 1.00 | 32.09 |
| ATOM | 9706 | CD1 | ILE | 5215 | 30.401 | 14.021 | 82.360 | 1.00 | 32.07 |
| ATOM | 9707 | C | ILE | 5215 | 34.204 | 11.847 | 83.058 | 1.00 | 32.78 |
| ATOM | 9708 | O | ILE | 5215 | 35.187 | 12.412 | 82.592 | 1.00 | 32.85 |
| ATOM | 9709 | N | ILE | 5216 | 33.775 | 10.674 | 82.623 | 1.00 | 32.21 |
| ATOM | 9710 | CA | ILE | 5216 | 34.439 | 9.996 | 81.526 | 1.00 | 31.79 |
| ATOM | 9711 | CB | ILE | 5216 | 35.035 | 8.677 | 82.003 | 1.00 | 31.89 |
| ATOM | 9712 | CG2 | ILE | 5216 | 35.644 | 7.923 | 80.842 | 1.00 | 30.65 |
| ATOM | 9713 | CG1 | ILE | 5216 | 36.084 | 8.970 | 83.072 | 1.00 | 33.24 |
| ATOM | 9714 | CD1 | ILE | 5216 | 36.552 | 7.742 | 83.840 | 1.00 | 34.55 |
| ATOM | 9715 | C | ILE | 5216 | 33.490 | 9.732 | 80.360 | 1.00 | 31.66 |
| ATOM | 9716 | O | ILE | 5216 | 32.371 | 9.244 | 80.540 | 1.00 | 31.63 |
| ATOM | 9717 | N | MSE | 5217 | 33.930 | 10.089 | 79.161 | 1.00 | 31.28 |
| ATOM | 9718 | CA | MSE | 5217 | 33.132 | 9.856 | 77.968 | 1.00 | 31.27 |
| ATOM | 9719 | CB | MSE | 5217 | 32.842 | 11.152 | 77.203 | 1.00 | 30.34 |
| ATOM | 9720 | CG | MSE | 5217 | 31.776 | 12.027 | 77.818 | 1.00 | 29.50 |
| ATOM | 9721 | SE | MSE | 5217 | 31.459 | 13.568 | 76.902 | 1.00 | 28.13 |
| ATOM | 9722 | CE | MSE | 5217 | 32.608 | 14.656 | 77.722 | 1.00 | 28.46 |
| ATOM | 9723 | C | MSE | 5217 | 33.964 | 8.951 | 77.101 | 1.00 | 31.69 |
| ATOM | 9724 | O | MSE | 5217 | 35.086 | 9.286 | 76.738 | 1.00 | 31.92 |
| ATOM | 9725 | N | ASP | 5218 | 33.415 | 7.788 | 76.787 | 1.00 | 32.64 |
| ATOM | 9726 | CA | ASP | 5218 | 34.105 | 6.823 | 75.955 | 1.00 | 32.71 |
| ATOM | 9727 | CB | ASP | 5218 | 33.656 | 5.416 | 76.379 | 1.00 | 34.17 |
| ATOM | 9728 | CG | ASP | 5218 | 34.570 | 4.324 | 75.860 | 1.00 | 36.63 |
| ATOM | 9729 | OD1 | ASP | 5218 | 34.186 | 3.642 | 74.888 | 1.00 | 37.52 |
| ATOM | 9730 | OD2 | ASP | 5218 | 35.678 | 4.147 | 76.418 | 1.00 | 38.86 |
| ATOM | 9731 | C | ASP | 5218 | 33.766 | 7.124 | 74.492 | 1.00 | 31.64 |
| ATOM | 9732 | O | ASP | 5218 | 32.662 | 7.568 | 74.185 | 1.00 | 31.91 |
| ATOM | 9733 | N | SER | 5219 | 34.742 | 6.921 | 73.615 | 1.00 | 31.36 |
| ATOM | 9734 | CA | SER | 5219 | 34.610 | 7.133 | 72.178 | 1.00 | 30.35 |
| ATOM | 9735 | CB | SER | 5219 | 33.950 | 5.899 | 71.574 | 1.00 | 30.24 |
| ATOM | 9736 | OG | SER | 5219 | 34.314 | 5.759 | 70.209 | 1.00 | 34.80 |
| ATOM | 9737 | C | SER | 5219 | 33.873 | 8.415 | 71.764 | 1.00 | 29.48 |
| ATOM | 9738 | O | SER | 5219 | 32.766 | 8.368 | 71.230 | 1.00 | 29.70 |
| ATOM | 9739 | N | VAL | 5220 | 34.499 | 9.562 | 72.001 | 1.00 | 28.38 |
| ATOM | 9740 | CA | VAL | 5220 | 33.888 | 10.833 | 71.650 | 1.00 | 27.54 |
| ATOM | 9741 | CB | VAL | 5220 | 34.767 | 11.980 | 72.103 | 1.00 | 27.07 |
| ATOM | 9742 | CG1 | VAL | 5220 | 34.795 | 12.008 | 73.624 | 1.00 | 27.24 |
| ATOM | 9743 | CG2 | VAL | 5220 | 36.170 | 11.812 | 71.539 | 1.00 | 26.92 |
| ATOM | 9744 | C | VAL | 5220 | 33.641 | 10.952 | 70.159 | 1.00 | 27.45 |
| ATOM | 9745 | O | VAL | 5220 | 34.363 | 10.379 | 69.358 | 1.00 | 28.70 |
| ATOM | 9746 | N | VAL | 5221 | 32.607 | 11.702 | 69.796 | 1.00 | 27.41 |
| ATOM | 9747 | CA | VAL | 5221 | 32.230 | 11.925 | 68.406 | 1.00 | 26.02 |
| ATOM | 9748 | CB | VAL | 5221 | 30.913 | 11.171 | 68.071 | 1.00 | 26.76 |
| ATOM | 9749 | CG1 | VAL | 5221 | 31.147 | 9.679 | 68.009 | 1.00 | 25.30 |
| ATOM | 9750 | CG2 | VAL | 5221 | 29.841 | 11.482 | 69.143 | 1.00 | 24.94 |
| ATOM | 9751 | C | VAL | 5221 | 31.966 | 13.420 | 68.243 | 1.00 | 26.31 |
| ATOM | 9752 | O | VAL | 5221 | 31.818 | 14.145 | 69.225 | 1.00 | 26.45 |
| ATOM | 9753 | N | PRO | 5222 | 31.884 | 13.892 | 67.000 | 1.00 | 26.22 |
| ATOM | 9754 | CD | PRO | 5222 | 31.902 | 13.062 | 65.780 | 1.00 | 25.49 |
| ATOM | 9755 | CA | PRO | 5222 | 31.631 | 15.301 | 66.691 | 1.00 | 26.25 |
| ATOM | 9756 | CB | PRO | 5222 | 31.238 | 15.246 | 65.218 | 1.00 | 26.04 |
| ATOM | 9757 | CG | PRO | 5222 | 32.040 | 14.090 | 64.693 | 1.00 | 25.59 |
| ATOM | 9758 | C | PRO | 5222 | 30.522 | 15.933 | 67.552 | 1.00 | 27.15 |
| ATOM | 9759 | O | PRO | 5222 | 30.647 | 17.072 | 68.005 | 1.00 | 27.54 |
| ATOM | 9760 | N | SER | 5223 | 29.436 | 15.197 | 67.776 | 1.00 | 27.49 |
| ATOM | 9761 | CA | SER | 5223 | 28.328 | 15.746 | 68.553 | 1.00 | 28.52 |
| ATOM | 9762 | CB | SER | 5223 | 27.044 | 14.914 | 68.374 | 1.00 | 27.47 |
| ATOM | 9763 | OG | SER | 5223 | 27.150 | 13.608 | 68.918 | 1.00 | 27.06 |
| ATOM | 9764 | C | SER | 5223 | 28.649 | 15.911 | 70.039 | 1.00 | 29.24 |
| ATOM | 9765 | O | SER | 5223 | 27.839 | 16.435 | 70.796 | 1.00 | 30.12 |
| ATOM | 9766 | N | ASP | 5224 | 29.826 | 15.473 | 70.462 | 1.00 | 29.62 |
| ATOM | 9767 | CA | ASP | 5224 | 30.210 | 15.634 | 71.855 | 1.00 | 30.12 |
| ATOM | 9768 | CB | ASP | 5224 | 31.185 | 14.529 | 72.303 | 1.00 | 30.29 |
| ATOM | 9769 | CG | ASP | 5224 | 30.507 | 13.161 | 72.477 | 1.00 | 30.34 |
| ATOM | 9770 | OD1 | ASP | 5224 | 29.369 | 13.109 | 72.985 | 1.00 | 28.80 |
| ATOM | 9771 | OD2 | ASP | 5224 | 31.126 | 12.136 | 72.120 | 1.00 | 29.23 |
| ATOM | 9772 | C | ASP | 5224 | 30.880 | 16.990 | 72.028 | 1.00 | 30.64 |
| ATOM | 9773 | O | ASP | 5224 | 31.113 | 17.429 | 73.151 | 1.00 | 31.26 |
| ATOM | 9774 | N | LYS | 5225 | 31.199 | 17.643 | 70.910 | 1.00 | 31.04 |
| ATOM | 9775 | CA | LYS | 5225 | 31.851 | 18.952 | 70.927 | 1.00 | 31.07 |
| ATOM | 9776 | CB | LYS | 5225 | 31.881 | 19.565 | 69.522 | 1.00 | 32.09 |
| ATOM | 9777 | CG | LYS | 5225 | 32.813 | 18.925 | 68.511 | 1.00 | 34.06 |
| ATOM | 9778 | CD | LYS | 5225 | 32.731 | 19.668 | 67.180 | 1.00 | 35.70 |
| ATOM | 9779 | CE | LYS | 5225 | 33.875 | 19.281 | 66.233 | 1.00 | 38.01 |
| ATOM | 9780 | NZ | LYS | 5225 | 34.054 | 20.269 | 65.105 | 1.00 | 39.20 |
| ATOM | 9781 | C | LYS | 5225 | 31.080 | 19.908 | 71.809 | 1.00 | 31.19 |
| ATOM | 9782 | O | LYS | 5225 | 29.855 | 19.953 | 71.739 | 1.00 | 30.95 |
| ATOM | 9783 | N | GLY | 5226 | 31.788 | 20.692 | 72.617 | 1.00 | 31.49 |
| ATOM | 9784 | CA | GLY | 5226 | 31.109 | 21.654 | 73.464 | 1.00 | 31.39 |
| ATOM | 9785 | C | GLY | 5226 | 31.851 | 22.016 | 74.730 | 1.00 | 32.44 |
| ATOM | 9786 | O | GLY | 5226 | 32.995 | 21.595 | 74.942 | 1.00 | 31.89 |
| ATOM | 9787 | N | ASN | 5227 | 31.200 | 22.818 | 75.570 | 1.00 | 32.87 |
| ATOM | 9788 | CA | ASN | 5227 | 31.789 | 23.237 | 76.836 | 1.00 | 33.42 |
| ATOM | 9789 | CB | ASN | 5227 | 31.465 | 24.704 | 77.144 | 1.00 | 34.21 |
| ATOM | 9790 | CG | ASN | 5227 | 32.238 | 25.676 | 76.268 | 1.00 | 35.06 |
| ATOM | 9791 | OD1 | ASN | 5227 | 33.459 | 25.578 | 76.130 | 1.00 | 36.16 |
| ATOM | 9792 | ND2 | ASN | 5227 | 31.530 | 26.632 | 75.686 | 1.00 | 35.29 |
| ATOM | 9793 | C | ASN | 5227 | 31.224 | 22.375 | 77.950 | 1.00 | 33.45 |
| ATOM | 9794 | O | ASN | 5227 | 30.013 | 22.187 | 78.054 | 1.00 | 34.52 |
| ATOM | 9795 | N | TYR | 5228 | 32.098 | 21.847 | 78.786 | 1.00 | 32.68 |
| ATOM | 9796 | CA | TYR | 5228 | 31.646 | 21.028 | 79.890 | 1.00 | 32.31 |
| ATOM | 9797 | CB | TYR | 5228 | 32.274 | 19.636 | 79.821 | 1.00 | 31.70 |
| ATOM | 9798 | CG | TYR | 5228 | 31.761 | 18.813 | 78.660 | 1.00 | 31.87 |
| ATOM | 9799 | CD1 | TYR | 5228 | 32.273 | 18.983 | 77.370 | 1.00 | 31.67 |
| ATOM | 9800 | CE1 | TYR | 5228 | 31.746 | 18.275 | 76.279 | 1.00 | 31.95 |
| ATOM | 9801 | CD2 | TYR | 5228 | 30.714 | 17.911 | 78.837 | 1.00 | 32.05 |
| ATOM | 9802 | CE2 | TYR | 5228 | 30.177 | 17.198 | 77.757 | 1.00 | 32.89 |
| ATOM | 9803 | CZ | TYR | 5228 | 30.695 | 17.385 | 76.482 | 1.00 | 32.38 |
| ATOM | 9804 | OH | TYR | 5228 | 30.148 | 16.692 | 75.424 | 1.00 | 31.48 |
| ATOM | 9805 | C | TYR | 5228 | 32.020 | 21.730 | 81.178 | 1.00 | 32.87 |
| ATOM | 9806 | O | TYR | 5228 | 33.194 | 22.022 | 81.426 | 1.00 | 31.99 |
| ATOM | 9807 | N | THR | 5229 | 31.003 | 22.009 | 81.986 | 1.00 | 33.54 |
| ATOM | 9808 | CA | THR | 5229 | 31.189 | 22.706 | 83.245 | 1.00 | 34.63 |
| ATOM | 9809 | CB | THR | 5229 | 30.289 | 23.928 | 83.320 | 1.00 | 33.92 |
| ATOM | 9810 | OG1 | THR | 5229 | 30.515 | 24.758 | 82.172 | 1.00 | 31.85 |
| ATOM | 9811 | CG2 | THR | 5229 | 30.563 | 24.697 | 84.611 | 1.00 | 32.94 |
| ATOM | 9812 | C | THR | 5229 | 30.865 | 21.840 | 84.438 | 1.00 | 35.98 |
| ATOM | 9813 | O | THR | 5229 | 29.820 | 21.198 | 84.486 | 1.00 | 37.53 |
| ATOM | 9814 | N | CYS | 5230 | 31.768 | 21.830 | 85.404 | 1.00 | 37.16 |
| ATOM | 9815 | CA | CYS | 5230 | 31.573 | 21.065 | 86.620 | 1.00 | 39.00 |
| ATOM | 9816 | CB | CYS | 5230 | 32.889 | 20.460 | 87.066 | 1.00 | 39.17 |
| ATOM | 9817 | SG | CYS | 5230 | 34.010 | 21.769 | 87.572 | 1.00 | 41.41 |
| ATOM | 9818 | C | CYS | 5230 | 31.133 | 22.073 | 87.677 | 1.00 | 39.83 |
| ATOM | 9819 | O | CYS | 5230 | 31.674 | 23.178 | 87.748 | 1.00 | 39.23 |
| ATOM | 9820 | N | ILE | 5231 | 30.156 | 21.691 | 88.487 | 1.00 | 40.94 |
| ATOM | 9821 | CA | ILE | 5231 | 29.673 | 22.560 | 89.548 | 1.00 | 42.59 |
| ATOM | 9822 | CB | ILE | 5231 | 28.207 | 22.965 | 89.307 | 1.00 | 42.87 |
| ATOM | 9823 | CG2 | ILE | 5231 | 27.650 | 23.662 | 90.535 | 1.00 | 43.49 |
| ATOM | 9824 | CG1 | ILE | 5231 | 28.122 | 23.877 | 88.078 | 1.00 | 42.58 |
| ATOM | 9825 | CD1 | ILE | 5231 | 26.714 | 24.304 | 87.729 | 1.00 | 41.47 |
| ATOM | 9826 | C | ILE | 5231 | 29.807 | 21.846 | 90.891 | 1.00 | 43.55 |
| ATOM | 9827 | O | ILE | 5231 | 29.102 | 20.876 | 91.164 | 1.00 | 43.37 |
| ATOM | 9828 | N | VAL | 5232 | 30.738 | 22.325 | 91.711 | 1.00 | 45.09 |
| ATOM | 9829 | CA | VAL | 5232 | 30.983 | 21.740 | 93.017 | 1.00 | 46.89 |
| ATOM | 9830 | CB | VAL | 5232 | 32.485 | 21.566 | 93.262 | 1.00 | 47.25 |
| ATOM | 9831 | CG1 | VAL | 5232 | 32.723 | 21.020 | 94.655 | 1.00 | 47.60 |
| ATOM | 9832 | CG2 | VAL | 5232 | 33.069 | 20.628 | 92.216 | 1.00 | 46.98 |
| ATOM | 9833 | C | VAL | 5232 | 30.400 | 22.663 | 94.071 | 1.00 | 48.40 |
| ATOM | 9834 | O | VAL | 5232 | 30.770 | 23.836 | 94.155 | 1.00 | 48.63 |
| ATOM | 9835 | N | GLU | 5233 | 29.484 | 22.141 | 94.878 | 1.00 | 50.11 |
| ATOM | 9836 | CA | GLU | 5233 | 28.869 | 22.974 | 95.900 | 1.00 | 51.75 |
| ATOM | 9837 | CB | GLU | 5233 | 27.699 | 23.766 | 95.285 | 1.00 | 53.36 |
| ATOM | 9838 | CG | GLU | 5233 | 26.496 | 22.928 | 94.822 | 1.00 | 55.76 |
| ATOM | 9839 | CD | GLU | 5233 | 25.434 | 23.753 | 94.065 | 1.00 | 58.15 |
| ATOM | 9840 | OE1 | GLU | 5233 | 24.285 | 23.269 | 93.914 | 1.00 | 59.04 |
| ATOM | 9841 | OE2 | GLU | 5233 | 25.745 | 24.882 | 93.607 | 1.00 | 58.84 |
| ATOM | 9842 | C | GLU | 5233 | 28.391 | 22.244 | 97.150 | 1.00 | 51.95 |
| ATOM | 9843 | O | GLU | 5233 | 28.168 | 21.031 | 97.145 | 1.00 | 51.51 |
| ATOM | 9844 | N | ASN | 5234 | 28.269 | 23.017 | 98.226 | 1.00 | 52.56 |
| ATOM | 9845 | CA | ASN | 5234 | 27.769 | 22.548 | 99.515 | 1.00 | 53.19 |
| ATOM | 9846 | CB | ASN | 5234 | 28.907 | 22.081 | 100.456 | 1.00 | 52.38 |
| ATOM | 9847 | CG | ASN | 5234 | 29.828 | 23.210 | 100.905 | 1.00 | 51.98 |
| ATOM | 9848 | OD1 | ASN | 5234 | 29.583 | 24.388 | 100.630 | 1.00 | 51.89 |
| ATOM | 9849 | ND2 | ASN | 5234 | 30.897 | 22.848 | 101.611 | 1.00 | 50.59 |
| ATOM | 9850 | C | ASN | 5234 | 27.015 | 23.740 | 100.107 | 1.00 | 54.28 |
| ATOM | 9851 | O | ASN | 5234 | 26.960 | 24.812 | 99.496 | 1.00 | 54.27 |
| ATOM | 9852 | N | GLU | 5235 | 26.432 | 23.562 | 101.284 | 1.00 | 55.32 |
| ATOM | 9853 | CA | GLU | 5235 | 25.675 | 24.633 | 101.913 | 1.00 | 56.54 |
| ATOM | 9854 | CB | GLU | 5235 | 25.193 | 24.179 | 103.295 | 1.00 | 58.33 |
| ATOM | 9855 | CG | GLU | 5235 | 24.547 | 25.285 | 104.111 | 1.00 | 61.05 |
| ATOM | 9856 | CD | GLU | 5235 | 24.261 | 24.855 | 105.531 | 1.00 | 62.98 |
| ATOM | 9857 | OE1 | GLU | 5235 | 25.064 | 24.050 | 106.059 | 1.00 | 63.62 |
| ATOM | 9858 | OE2 | GLU | 5235 | 23.253 | 25.327 | 106.118 | 1.00 | 63.53 |
| ATOM | 9859 | C | GLU | 5235 | 26.422 | 25.967 | 102.040 | 1.00 | 56.45 |
| ATOM | 9860 | O | GLU | 5235 | 25.797 | 27.028 | 102.058 | 1.00 | 56.01 |
| ATOM | 9861 | N | TYR | 5236 | 27.750 | 25.923 | 102.107 | 1.00 | 56.65 |
| ATOM | 9862 | CA | TYR | 5236 | 28.529 | 27.151 | 102.271 | 1.00 | 56.71 |
| ATOM | 9863 | CB | TYR | 5236 | 29.642 | 26.927 | 103.287 | 1.00 | 58.12 |
| ATOM | 9864 | CG | TYR | 5236 | 29.114 | 26.299 | 104.539 | 1.00 | 60.43 |
| ATOM | 9865 | CD1 | TYR | 5236 | 29.152 | 24.912 | 104.712 | 1.00 | 61.38 |
| ATOM | 9866 | CE1 | TYR | 5236 | 28.580 | 24.315 | 105.829 | 1.00 | 62.15 |
| ATOM | 9867 | CD2 | TYR | 5236 | 28.491 | 27.076 | 105.520 | 1.00 | 61.21 |
| ATOM | 9868 | CE2 | TYR | 5236 | 27.914 | 26.491 | 106.640 | 1.00 | 62.02 |
| ATOM | 9869 | CZ | TYR | 5236 | 27.961 | 25.110 | 106.787 | 1.00 | 62.48 |
| ATOM | 9870 | OH | TYR | 5236 | 27.378 | 24.519 | 107.883 | 1.00 | 63.37 |
| ATOM | 9871 | C | TYR | 5236 | 29.119 | 27.797 | 101.031 | 1.00 | 56.17 |
| ATOM | 9872 | O | TYR | 5236 | 29.779 | 28.830 | 101.135 | 1.00 | 56.53 |
| ATOM | 9873 | N | GLY | 5237 | 28.895 | 27.216 | 99.860 | 1.00 | 55.21 |
| ATOM | 9874 | CA | GLY | 5237 | 29.447 | 27.828 | 98.666 | 1.00 | 53.83 |
| ATOM | 9875 | C | GLY | 5237 | 29.490 | 26.927 | 97.454 | 1.00 | 52.95 |
| ATOM | 9876 | O | GLY | 5237 | 29.319 | 25.707 | 97.553 | 1.00 | 53.09 |
| ATOM | 9877 | N | SER | 5238 | 29.739 | 27.538 | 96.301 | 1.00 | 51.67 |
| ATOM | 9878 | CA | SER | 5238 | 29.790 | 26.804 | 95.048 | 1.00 | 50.10 |
| ATOM | 9879 | CB | SER | 5238 | 28.419 | 26.885 | 94.362 | 1.00 | 49.82 |
| ATOM | 9880 | OG | SER | 5238 | 28.424 | 26.225 | 93.109 | 1.00 | 50.06 |
| ATOM | 9881 | C | SER | 5238 | 30.869 | 27.347 | 94.116 | 1.00 | 48.81 |
| ATOM | 9882 | O | SER | 5238 | 31.006 | 28.556 | 93.944 | 1.00 | 48.57 |
| ATOM | 9883 | N | ILE | 5239 | 31.626 | 26.435 | 93.520 | 1.00 | 47.16 |
| ATOM | 9884 | CA | ILE | 5239 | 32.687 | 26.781 | 92.593 | 1.00 | 45.37 |
| ATOM | 9885 | CB | ILE | 5239 | 34.052 | 26.359 | 93.169 | 1.00 | 45.09 |
| ATOM | 9886 | CG2 | ILE | 5239 | 34.340 | 27.142 | 94.440 | 1.00 | 45.08 |
| ATOM | 9887 | CG1 | ILE | 5239 | 34.042 | 24.866 | 93.497 | 1.00 | 44.82 |
| ATOM | 9888 | CD1 | ILE | 5239 | 35.295 | 24.380 | 94.191 | 1.00 | 44.96 |
| ATOM | 9889 | C | ILE | 5239 | 32.420 | 26.039 | 91.284 | 1.00 | 44.96 |
| ATOM | 9890 | O | ILE | 5239 | 31.582 | 25.127 | 91.234 | 1.00 | 44.71 |
| ATOM | 9891 | N | ASN | 5240 | 33.124 | 26.425 | 90.227 | 1.00 | 44.15 |
| ATOM | 9892 | CA | ASN | 5240 | 32.937 | 25.778 | 88.943 | 1.00 | 43.48 |
| ATOM | 9893 | CB | ASN | 5240 | 31.647 | 26.269 | 88.295 | 1.00 | 43.32 |
| ATOM | 9894 | CG | ASN | 5240 | 31.650 | 27.753 | 88.073 | 1.00 | 43.34 |
| ATOM | 9895 | OD1 | ASN | 5240 | 32.595 | 28.307 | 87.508 | 1.00 | 43.80 |
| ATOM | 9896 | ND2 | ASN | 5240 | 30.592 | 28.415 | 88.512 | 1.00 | 43.66 |
| ATOM | 9897 | C | ASN | 5240 | 34.103 | 26.023 | 88.002 | 1.00 | 43.08 |
| ATOM | 9898 | O | ASN | 5240 | 34.902 | 26.935 | 88.205 | 1.00 | 43.02 |
| ATOM | 9899 | N | HIS | 5241 | 34.183 | 25.199 | 86.964 | 1.00 | 42.56 |
| ATOM | 9900 | CA | HIS | 5241 | 35.238 | 25.306 | 85.981 | 1.00 | 41.61 |
| ATOM | 9901 | CB | HIS | 5241 | 36.431 | 24.479 | 86.435 | 1.00 | 42.31 |
| ATOM | 9902 | CG | HIS | 5241 | 37.659 | 24.696 | 85.614 | 1.00 | 43.44 |
| ATOM | 9903 | CD2 | HIS | 5241 | 38.404 | 23.836 | 84.882 | 1.00 | 43.90 |
| ATOM | 9904 | ND1 | HIS | 5241 | 38.267 | 25.926 | 85.498 | 1.00 | 44.30 |
| ATOM | 9905 | CE1 | HIS | 5241 | 39.336 | 25.815 | 84.730 | 1.00 | 45.15 |
| ATOM | 9906 | NE2 | HIS | 5241 | 39.442 | 24.556 | 84.343 | 1.00 | 44.67 |
| ATOM | 9907 | C | HIS | 5241 | 34.673 | 24.767 | 84.679 | 1.00 | 41.15 |
| ATOM | 9908 | O | HIS | 5241 | 33.808 | 23.888 | 84.689 | 1.00 | 41.21 |
| ATOM | 9909 | N | THR | 5242 | 35.152 | 25.289 | 83.558 | 1.00 | 40.25 |
| ATOM | 9910 | CA | THR | 5242 | 34.657 | 24.846 | 82.265 | 1.00 | 38.75 |
| ATOM | 9911 | CB | THR | 5242 | 33.848 | 25.950 | 81.599 | 1.00 | 38.23 |
| ATOM | 9912 | OG1 | THR | 5242 | 32.710 | 26.242 | 82.409 | 1.00 | 37.99 |
| ATOM | 9913 | CG2 | THR | 5242 | 33.393 | 25.519 | 80.211 | 1.00 | 38.76 |
| ATOM | 9914 | C | THR | 5242 | 35.760 | 24.415 | 81.313 | 1.00 | 37.77 |
| ATOM | 9915 | O | THR | 5242 | 36.752 | 25.117 | 81.133 | 1.00 | 38.85 |
| ATOM | 9916 | N | TYR | 5243 | 35.590 | 23.247 | 80.712 | 1.00 | 36.67 |
| ATOM | 9917 | CA | TYR | 5243 | 36.566 | 22.744 | 79.755 | 1.00 | 35.67 |
| ATOM | 9918 | CB | TYR | 5243 | 36.986 | 21.304 | 80.073 | 1.00 | 34.02 |
| ATOM | 9919 | CG | TYR | 5243 | 37.654 | 21.122 | 81.415 | 1.00 | 33.64 |
| ATOM | 9920 | CD1 | TYR | 5243 | 36.945 | 20.625 | 82.510 | 1.00 | 33.18 |
| ATOM | 9921 | CE1 | TYR | 5243 | 37.559 | 20.449 | 83.742 | 1.00 | 32.45 |
| ATOM | 9922 | CD2 | TYR | 5243 | 39.001 | 21.440 | 81.593 | 1.00 | 33.37 |
| ATOM | 9923 | CE2 | TYR | 5243 | 39.623 | 21.266 | 82.820 | 1.00 | 32.30 |
| ATOM | 9924 | CZ | TYR | 5243 | 38.897 | 20.772 | 83.890 | 1.00 | 32.53 |
| ATOM | 9925 | OH | TYR | 5243 | 39.513 | 20.612 | 85.108 | 1.00 | 32.64 |
| ATOM | 9926 | C | TYR | 5243 | 35.922 | 22.768 | 78.382 | 1.00 | 35.58 |
| ATOM | 9927 | O | TYR | 5243 | 34.697 | 22.651 | 78.255 | 1.00 | 36.05 |
| ATOM | 9928 | N | GLN | 5244 | 36.733 | 22.942 | 77.348 | 1.00 | 35.13 |
| ATOM | 9929 | CA | GLN | 5244 | 36.179 | 22.939 | 76.010 | 1.00 | 34.91 |
| ATOM | 9930 | CB | GLN | 5244 | 36.670 | 24.133 | 75.189 | 1.00 | 34.76 |
| ATOM | 9931 | CG | GLN | 5244 | 35.682 | 24.504 | 74.079 | 1.00 | 37.13 |
| ATOM | 9932 | CD | GLN | 5244 | 36.320 | 25.245 | 72.920 | 1.00 | 37.92 |
| ATOM | 9933 | OE1 | GLN | 5244 | 37.080 | 26.194 | 73.115 | 1.00 | 38.64 |
| ATOM | 9934 | NE2 | GLN | 5244 | 36.006 | 24.814 | 71.699 | 1.00 | 37.38 |
| ATOM | 9935 | C | GLN | 5244 | 36.612 | 21.650 | 75.345 | 1.00 | 33.54 |
| ATOM | 9936 | O | GLN | 5244 | 37.781 | 21.293 | 75.388 | 1.00 | 33.39 |
| ATOM | 9937 | N | LEU | 5245 | 35.663 | 20.934 | 74.757 | 1.00 | 33.18 |
| ATOM | 9938 | CA | LEU | 5245 | 35.992 | 19.693 | 74.070 | 1.00 | 32.06 |
| ATOM | 9939 | CB | LEU | 5245 | 35.112 | 18.537 | 74.540 | 1.00 | 31.09 |
| ATOM | 9940 | CG | LEU | 5245 | 35.248 | 17.263 | 73.688 | 1.00 | 31.16 |
| ATOM | 9941 | CD1 | LEU | 5245 | 36.670 | 16.738 | 73.734 | 1.00 | 31.52 |
| ATOM | 9942 | CD2 | LEU | 5245 | 34.289 | 16.204 | 74.198 | 1.00 | 30.73 |
| ATOM | 9943 | C | LEU | 5245 | 35.831 | 19.848 | 72.572 | 1.00 | 31.49 |
| ATOM | 9944 | O | LEU | 5245 | 34.773 | 20.239 | 72.081 | 1.00 | 30.84 |
| ATOM | 9945 | N | ASP | 5246 | 36.901 | 19.557 | 71.846 | 1.00 | 31.94 |
| ATOM | 9946 | CA | ASP | 5246 | 36.853 | 19.622 | 70.395 | 1.00 | 32.18 |
| ATOM | 9947 | CB | ASP | 5246 | 37.747 | 20.733 | 69.835 | 1.00 | 33.12 |
| ATOM | 9948 | CG | ASP | 5246 | 37.449 | 21.038 | 68.365 | 1.00 | 34.48 |
| ATOM | 9949 | OD1 | ASP | 5246 | 38.108 | 21.928 | 67.792 | 1.00 | 34.54 |
| ATOM | 9950 | OD2 | ASP | 5246 | 36.552 | 20.393 | 67.769 | 1.00 | 35.19 |
| ATOM | 9951 | C | ASP | 5246 | 37.294 | 18.271 | 69.859 | 1.00 | 31.81 |
| ATOM | 9952 | O | ASP | 5246 | 38.341 | 17.742 | 70.238 | 1.00 | 31.82 |
| ATOM | 9953 | N | VAL | 5247 | 36.460 | 17.714 | 68.991 | 1.00 | 31.54 |
| ATOM | 9954 | CA | VAL | 5247 | 36.704 | 16.424 | 68.388 | 1.00 | 31.54 |
| ATOM | 9955 | CB | VAL | 5247 | 35.459 | 15.557 | 68.508 | 1.00 | 31.34 |
| ATOM | 9956 | CG1 | VAL | 5247 | 35.629 | 14.266 | 67.721 | 1.00 | 31.07 |
| ATOM | 9957 | CG2 | VAL | 5247 | 35.198 | 15.278 | 69.965 | 1.00 | 31.19 |
| ATOM | 9958 | C | VAL | 5247 | 37.017 | 16.631 | 66.932 | 1.00 | 32.27 |
| ATOM | 9959 | O | VAL | 5247 | 36.239 | 17.248 | 66.217 | 1.00 | 32.16 |
| ATOM | 9960 | N | VAL | 5248 | 38.152 | 16.110 | 66.486 | 1.00 | 33.49 |
| ATOM | 9961 | CA | VAL | 5248 | 38.532 | 16.286 | 65.098 | 1.00 | 33.39 |
| ATOM | 9962 | CB | VAL | 5248 | 39.900 | 16.985 | 65.007 | 1.00 | 33.39 |
| ATOM | 9963 | CG1 | VAL | 5248 | 40.205 | 17.355 | 63.575 | 1.00 | 33.94 |
| ATOM | 9964 | CG2 | VAL | 5248 | 39.880 | 18.240 | 65.878 | 1.00 | 32.82 |
| ATOM | 9965 | C | VAL | 5248 | 38.499 | 14.996 | 64.308 | 1.00 | 33.46 |
| ATOM | 9966 | O | VAL | 5248 | 39.070 | 13.968 | 64.697 | 1.00 | 34.44 |
| ATOM | 9967 | N | GLU | 5249 | 37.737 | 15.099 | 63.220 | 1.00 | 33.71 |
| ATOM | 9968 | CA | GLU | 5249 | 37.512 | 14.043 | 62.239 | 1.00 | 33.52 |
| ATOM | 9969 | CB | GLU | 5249 | 36.116 | 14.191 | 61.630 | 1.00 | 34.17 |
| ATOM | 9970 | CG | GLU | 5249 | 34.978 | 13.735 | 62.520 | 1.00 | 36.62 |
| ATOM | 9971 | CD | GLU | 5249 | 33.621 | 13.839 | 61.829 | 1.00 | 38.82 |
| ATOM | 9972 | OE1 | GLU | 5249 | 33.014 | 14.941 | 61.839 | 1.00 | 39.53 |
| ATOM | 9973 | OE2 | GLU | 5249 | 33.165 | 12.818 | 61.262 | 1.00 | 38.96 |
| ATOM | 9974 | C | GLU | 5249 | 38.551 | 14.164 | 61.128 | 1.00 | 32.43 |
| ATOM | 9975 | O | GLU | 5249 | 38.834 | 15.253 | 60.660 | 1.00 | 32.12 |
| ATOM | 9976 | N | ARG | 5250 | 39.113 | 13.044 | 60.702 | 1.00 | 32.13 |
| ATOM | 9977 | CA | ARG | 5250 | 40.119 | 13.060 | 59.649 | 1.00 | 31.75 |
| ATOM | 9978 | CB | ARG | 5250 | 41.381 | 12.330 | 60.131 | 1.00 | 30.63 |
| ATOM | 9979 | CG | ARG | 5250 | 41.941 | 12.810 | 61.484 | 1.00 | 30.28 |
| ATOM | 9980 | CD | ARG | 5250 | 42.137 | 14.326 | 61.557 | 1.00 | 28.58 |
| ATOM | 9981 | NE | ARG | 5250 | 42.969 | 14.828 | 60.468 | 1.00 | 28.92 |
| ATOM | 9982 | CZ | ARG | 5250 | 44.298 | 14.827 | 60.473 | 1.00 | 28.27 |
| ATOM | 9983 | NH1 | ARG | 5250 | 44.966 | 14.356 | 61.518 | 1.00 | 29.13 |
| ATOM | 9984 | NH2 | ARG | 5250 | 44.955 | 15.290 | 59.431 | 1.00 | 25.77 |
| ATOM | 9985 | C | ARG | 5250 | 39.577 | 12.375 | 58.390 | 1.00 | 31.87 |
| ATOM | 9986 | O | ARG | 5250 | 38.610 | 11.612 | 58.463 | 1.00 | 31.95 |
| ATOM | 9987 | N | SER | 5251 | 40.180 | 12.670 | 57.240 | 1.00 | 31.52 |
| ATOM | 9988 | CA | SER | 5251 | 39.785 | 12.042 | 55.977 | 1.00 | 31.27 |
| ATOM | 9989 | CB | SER | 5251 | 39.200 | 13.055 | 55.000 | 1.00 | 32.27 |
| ATOM | 9990 | OG | SER | 5251 | 38.123 | 13.742 | 55.592 | 1.00 | 33.93 |
| ATOM | 9991 | C | SER | 5251 | 41.050 | 11.457 | 55.389 | 1.00 | 30.49 |
| ATOM | 9992 | O | SER | 5251 | 41.699 | 12.071 | 54.548 | 1.00 | 30.34 |
| ATOM | 9993 | N | PRO | 5252 | 41.412 | 10.248 | 55.829 | 1.00 | 30.39 |
| ATOM | 9994 | CD | PRO | 5252 | 40.629 | 9.390 | 56.736 | 1.00 | 30.25 |
| ATOM | 9995 | CA | PRO | 5252 | 42.607 | 9.551 | 55.372 | 1.00 | 30.54 |
| ATOM | 9996 | CB | PRO | 5252 | 42.780 | 8.476 | 56.432 | 1.00 | 30.30 |
| ATOM | 9997 | CG | PRO | 5252 | 41.388 | 8.075 | 56.684 | 1.00 | 30.05 |
| ATOM | 9998 | C | PRO | 5252 | 42.458 | 8.978 | 53.970 | 1.00 | 30.72 |
| ATOM | 9999 | O | PRO | 5252 | 42.481 | 7.767 | 53.785 | 1.00 | 30.53 |
| ATOM | 10000 | N | HIS | 5253 | 42.300 | 9.852 | 52.984 | 1.00 | 31.71 |
| ATOM | 10001 | CA | HIS | 5253 | 42.165 | 9.404 | 51.605 | 1.00 | 32.43 |
| ATOM | 10002 | CB | HIS | 5253 | 40.686 | 9.258 | 51.209 | 1.00 | 33.92 |
| ATOM | 10003 | CG | HIS | 5253 | 39.870 | 10.496 | 51.423 | 1.00 | 37.39 |
| ATOM | 10004 | CD2 | HIS | 5253 | 38.684 | 10.684 | 52.052 | 1.00 | 38.35 |
| ATOM | 10005 | ND1 | HIS | 5253 | 40.239 | 11.732 | 50.928 | 1.00 | 38.54 |
| ATOM | 10006 | CE1 | HIS | 5253 | 39.315 | 12.625 | 51.240 | 1.00 | 39.02 |
| ATOM | 10007 | NE2 | HIS | 5253 | 38.360 | 12.016 | 51.924 | 1.00 | 38.92 |
| ATOM | 10008 | C | HIS | 5253 | 42.872 | 10.354 | 50.647 | 1.00 | 31.48 |
| ATOM | 10009 | O | HIS | 5253 | 43.209 | 11.487 | 51.011 | 1.00 | 31.12 |
| ATOM | 10010 | N | ARG | 5254 | 43.111 | 9.880 | 49.431 | 1.00 | 29.92 |
| ATOM | 10011 | CA | ARG | 5254 | 43.764 | 10.694 | 48.425 | 1.00 | 29.03 |
| ATOM | 10012 | CB | ARG | 5254 | 43.978 | 9.891 | 47.143 | 1.00 | 30.01 |
| ATOM | 10013 | CG | ARG | 5254 | 42.706 | 9.380 | 46.496 | 1.00 | 30.64 |
| ATOM | 10014 | CD | ARG | 5254 | 43.050 | 8.564 | 45.263 | 1.00 | 32.33 |
| ATOM | 10015 | NE | ARG | 5254 | 41.848 | 8.049 | 44.616 | 1.00 | 35.15 |
| ATOM | 10016 | CZ | ARG | 5254 | 41.552 | 8.215 | 43.328 | 1.00 | 35.61 |
| ATOM | 10017 | NH1 | ARG | 5254 | 42.371 | 8.887 | 42.529 | 1.00 | 35.75 |
| ATOM | 10018 | NH2 | ARG | 5254 | 40.429 | 7.710 | 42.837 | 1.00 | 36.88 |
| ATOM | 10019 | C | ARG | 5254 | 42.889 | 11.915 | 48.148 | 1.00 | 27.73 |
| ATOM | 10020 | O | ARG | 5254 | 41.713 | 11.939 | 48.513 | 1.00 | 27.41 |
| ATOM | 10021 | N | PRO | 5255 | 43.449 | 12.941 | 47.500 | 1.00 | 26.38 |
| ATOM | 10022 | CD | PRO | 5255 | 44.814 | 13.081 | 46.965 | 1.00 | 25.27 |
| ATOM | 10023 | CA | PRO | 5255 | 42.660 | 14.145 | 47.210 | 1.00 | 25.76 |
| ATOM | 10024 | CB | PRO | 5255 | 43.653 | 15.052 | 46.469 | 1.00 | 24.66 |
| ATOM | 10025 | CG | PRO | 5255 | 44.986 | 14.574 | 46.928 | 1.00 | 23.72 |
| ATOM | 10026 | C | PRO | 5255 | 41.437 | 13.840 | 46.355 | 1.00 | 25.19 |
| ATOM | 10027 | O | PRO | 5255 | 41.445 | 12.910 | 45.558 | 1.00 | 25.22 |
| ATOM | 10028 | N | ILE | 5256 | 40.387 | 14.630 | 46.526 | 1.00 | 24.93 |
| ATOM | 10029 | CA | ILE | 5256 | 39.179 | 14.446 | 45.751 | 1.00 | 24.43 |
| ATOM | 10030 | CB | ILE | 5256 | 37.996 | 14.101 | 46.658 | 1.00 | 24.20 |
| ATOM | 10031 | CG2 | ILE | 5256 | 36.715 | 13.991 | 45.824 | 1.00 | 23.19 |
| ATOM | 10032 | CG1 | ILE | 5256 | 38.306 | 12.794 | 47.394 | 1.00 | 23.34 |
| ATOM | 10033 | CD1 | ILE | 5256 | 37.154 | 12.220 | 48.158 | 1.00 | 24.26 |
| ATOM | 10034 | C | ILE | 5256 | 38.905 | 15.712 | 44.965 | 1.00 | 24.81 |
| ATOM | 10035 | O | ILE | 5256 | 38.896 | 16.807 | 45.519 | 1.00 | 24.92 |
| ATOM | 10036 | N | LEU | 5257 | 38.707 | 15.557 | 43.662 | 1.00 | 25.21 |
| ATOM | 10037 | CA | LEU | 5257 | 38.444 | 16.693 | 42.784 | 1.00 | 25.92 |
| ATOM | 10038 | CB | LEU | 5257 | 39.205 | 16.520 | 41.466 | 1.00 | 24.96 |
| ATOM | 10039 | CG | LEU | 5257 | 40.683 | 16.091 | 41.563 | 1.00 | 25.78 |
| ATOM | 10040 | CD1 | LEU | 5257 | 41.312 | 16.140 | 40.187 | 1.00 | 26.13 |
| ATOM | 10041 | CD2 | LEU | 5257 | 41.447 | 16.977 | 42.506 | 1.00 | 23.35 |
| ATOM | 10042 | C | LEU | 5257 | 36.945 | 16.802 | 42.509 | 1.00 | 27.08 |
| ATOM | 10043 | O | LEU | 5257 | 36.280 | 15.794 | 42.272 | 1.00 | 26.56 |
| ATOM | 10044 | N | GLN | 5258 | 36.415 | 18.020 | 42.546 | 1.00 | 28.00 |
| ATOM | 10045 | CA | GLN | 5258 | 34.992 | 18.263 | 42.310 | 1.00 | 29.50 |
| ATOM | 10046 | CB | GLN | 5258 | 34.720 | 19.772 | 42.453 | 1.00 | 30.10 |
| ATOM | 10047 | CG | GLN | 5258 | 33.244 | 20.209 | 42.628 | 1.00 | 32.78 |
| ATOM | 10048 | CD | GLN | 5258 | 33.089 | 21.748 | 42.862 | 1.00 | 34.25 |
| ATOM | 10049 | OE1 | GLN | 5258 | 33.646 | 22.320 | 43.820 | 1.00 | 34.82 |
| ATOM | 10050 | NE2 | GLN | 5258 | 32.328 | 22.401 | 41.992 | 1.00 | 33.43 |
| ATOM | 10051 | C | GLN | 5258 | 34.600 | 17.766 | 40.910 | 1.00 | 29.74 |
| ATOM | 10052 | O | GLN | 5258 | 35.183 | 18.183 | 39.912 | 1.00 | 30.28 |
| ATOM | 10053 | N | ALA | 5259 | 33.627 | 16.863 | 40.848 | 1.00 | 29.19 |
| ATOM | 10054 | CA | ALA | 5259 | 33.164 | 16.335 | 39.577 | 1.00 | 28.90 |
| ATOM | 10055 | CB | ALA | 5259 | 31.938 | 15.466 | 39.805 | 1.00 | 28.95 |
| ATOM | 10056 | C | ALA | 5259 | 32.831 | 17.482 | 38.619 | 1.00 | 29.09 |
| ATOM | 10057 | O | ALA | 5259 | 32.288 | 18.509 | 39.034 | 1.00 | 29.26 |
| ATOM | 10058 | N | GLY | 5260 | 33.166 | 17.314 | 37.342 | 1.00 | 28.87 |
| ATOM | 10059 | CA | GLY | 5260 | 32.866 | 18.350 | 36.364 | 1.00 | 29.36 |
| ATOM | 10060 | C | GLY | 5260 | 33.943 | 19.385 | 36.072 | 1.00 | 29.16 |
| ATOM | 10061 | O | GLY | 5260 | 33.856 | 20.088 | 35.073 | 1.00 | 29.08 |
| ATOM | 10062 | N | LEU | 5261 | 34.955 | 19.487 | 36.924 | 1.00 | 29.15 |
| ATOM | 10063 | CA | LEU | 5261 | 36.011 | 20.463 | 36.728 | 1.00 | 28.81 |
| ATOM | 10064 | CB | LEU | 5261 | 35.997 | 21.483 | 37.873 | 1.00 | 27.44 |
| ATOM | 10065 | CG | LEU | 5261 | 34.648 | 22.175 | 38.138 | 1.00 | 27.66 |
| ATOM | 10066 | CD1 | LEU | 5261 | 34.777 | 23.147 | 39.310 | 1.00 | 25.56 |
| ATOM | 10067 | CD2 | LEU | 5261 | 34.171 | 22.907 | 36.886 | 1.00 | 25.80 |
| ATOM | 10068 | C | LEU | 5261 | 37.388 | 19.816 | 36.637 | 1.00 | 29.09 |
| ATOM | 10069 | O | LEU | 5261 | 37.708 | 18.914 | 37.403 | 1.00 | 29.60 |
| ATOM | 10070 | N | PRO | 5262 | 38.227 | 20.273 | 35.693 | 1.00 | 29.52 |
| ATOM | 10071 | CD | PRO | 5262 | 39.586 | 19.733 | 35.492 | 1.00 | 29.66 |
| ATOM | 10072 | CA | PRO | 5262 | 37.947 | 21.347 | 34.731 | 1.00 | 29.39 |
| ATOM | 10073 | CB | PRO | 5262 | 39.324 | 21.659 | 34.155 | 1.00 | 28.77 |
| ATOM | 10074 | CG | PRO | 5262 | 39.972 | 20.312 | 34.134 | 1.00 | 29.76 |
| ATOM | 10075 | C | PRO | 5262 | 36.956 | 20.880 | 33.679 | 1.00 | 29.37 |
| ATOM | 10076 | O | PRO | 5262 | 36.768 | 19.686 | 33.489 | 1.00 | 30.56 |
| ATOM | 10077 | N | ALA | 5263 | 36.326 | 21.825 | 32.994 | 1.00 | 30.06 |
| ATOM | 10078 | CA | ALA | 5263 | 35.333 | 21.499 | 31.978 | 1.00 | 30.27 |
| ATOM | 10079 | CB | ALA | 5263 | 34.079 | 22.312 | 32.230 | 1.00 | 28.22 |
| ATOM | 10080 | C | ALA | 5263 | 35.829 | 21.747 | 30.563 | 1.00 | 30.58 |
| ATOM | 10081 | O | ALA | 5263 | 36.667 | 22.612 | 30.342 | 1.00 | 30.67 |
| ATOM | 10082 | N | ASN | 5264 | 35.309 | 20.979 | 29.608 | 1.00 | 32.00 |
| ATOM | 10083 | CA | ASN | 5264 | 35.679 | 21.143 | 28.205 | 1.00 | 33.24 |
| ATOM | 10084 | CB | ASN | 5264 | 34.975 | 20.096 | 27.343 | 1.00 | 31.59 |
| ATOM | 10085 | CG | ASN | 5264 | 35.388 | 18.688 | 27.696 | 1.00 | 31.51 |
| ATOM | 10086 | OD1 | ASN | 5264 | 36.538 | 18.438 | 28.040 | 1.00 | 31.48 |
| ATOM | 10087 | ND2 | ASN | 5264 | 34.457 | 17.755 | 27.597 | 1.00 | 31.40 |
| ATOM | 10088 | C | ASN | 5264 | 35.286 | 22.546 | 27.737 | 1.00 | 34.46 |
| ATOM | 10089 | O | ASN | 5264 | 34.253 | 23.076 | 28.136 | 1.00 | 34.91 |
| ATOM | 10090 | N | LYS | 5265 | 36.112 | 23.151 | 26.901 | 1.00 | 35.80 |
| ATOM | 10091 | CA | LYS | 5265 | 35.815 | 24.484 | 26.418 | 1.00 | 37.56 |
| ATOM | 10092 | CB | LYS | 5265 | 36.595 | 25.527 | 27.224 | 1.00 | 38.04 |
| ATOM | 10093 | CG | LYS | 5265 | 36.184 | 25.596 | 28.686 | 1.00 | 40.06 |
| ATOM | 10094 | CD | LYS | 5265 | 37.040 | 26.577 | 29.474 | 1.00 | 41.54 |
| ATOM | 10095 | CE | LYS | 5265 | 36.533 | 26.784 | 30.908 | 1.00 | 41.47 |
| ATOM | 10096 | NZ | LYS | 5265 | 36.742 | 25.618 | 31.819 | 1.00 | 43.24 |
| ATOM | 10097 | C | LYS | 5265 | 36.113 | 24.637 | 24.936 | 1.00 | 38.23 |
| ATOM | 10098 | O | LYS | 5265 | 37.107 | 24.119 | 24.428 | 1.00 | 38.73 |
| ATOM | 10099 | N | THR | 5266 | 35.216 | 25.335 | 24.251 | 1.00 | 38.57 |
| ATOM | 10100 | CA | THR | 5266 | 35.352 | 25.611 | 22.834 | 1.00 | 38.83 |
| ATOM | 10101 | CB | THR | 5266 | 34.115 | 25.164 | 22.051 | 1.00 | 38.15 |
| ATOM | 10102 | OG1 | THR | 5266 | 33.997 | 23.744 | 22.110 | 1.00 | 38.74 |
| ATOM | 10103 | CG2 | THR | 5266 | 34.224 | 25.590 | 20.604 | 1.00 | 38.24 |
| ATOM | 10104 | C | THR | 5266 | 35.439 | 27.123 | 22.747 | 1.00 | 39.24 |
| ATOM | 10105 | O | THR | 5266 | 34.491 | 27.811 | 23.103 | 1.00 | 38.97 |
| ATOM | 10106 | N | VAL | 5267 | 36.568 | 27.649 | 22.287 | 1.00 | 39.70 |
| ATOM | 10107 | CA | VAL | 5267 | 36.717 | 29.092 | 22.192 | 1.00 | 40.05 |
| ATOM | 10108 | CB | VAL | 5267 | 37.634 | 29.625 | 23.306 | 1.00 | 39.06 |
| ATOM | 10109 | CG1 | VAL | 5267 | 37.064 | 29.262 | 24.649 | 1.00 | 38.38 |
| ATOM | 10110 | CG2 | VAL | 5267 | 39.027 | 29.052 | 23.153 | 1.00 | 38.44 |
| ATOM | 10111 | C | VAL | 5267 | 37.283 | 29.545 | 20.863 | 1.00 | 40.88 |
| ATOM | 10112 | O | VAL | 5267 | 37.903 | 28.770 | 20.136 | 1.00 | 40.45 |
| ATOM | 10113 | N | ALA | 5268 | 37.063 | 30.818 | 20.557 | 1.00 | 42.63 |
| ATOM | 10114 | CA | ALA | 5268 | 37.555 | 31.407 | 19.319 | 1.00 | 44.27 |
| ATOM | 10115 | CB | ALA | 5268 | 36.768 | 32.659 | 18.993 | 1.00 | 44.43 |
| ATOM | 10116 | C | ALA | 5268 | 39.028 | 31.744 | 19.476 | 1.00 | 45.10 |
| ATOM | 10117 | O | ALA | 5268 | 39.498 | 32.053 | 20.575 | 1.00 | 45.41 |
| ATOM | 10118 | N | LEU | 5269 | 39.760 | 31.669 | 18.375 | 1.00 | 45.93 |
| ATOM | 10119 | CA | LEU | 5269 | 41.180 | 31.975 | 18.390 | 1.00 | 46.94 |
| ATOM | 10120 | CB | LEU | 5269 | 41.694 | 32.002 | 16.953 | 1.00 | 47.16 |
| ATOM | 10121 | CG | LEU | 5269 | 42.946 | 31.202 | 16.598 | 1.00 | 47.79 |
| ATOM | 10122 | CD1 | LEU | 5269 | 43.315 | 31.528 | 15.157 | 1.00 | 47.37 |
| ATOM | 10123 | CD2 | LEU | 5269 | 44.102 | 31.545 | 17.543 | 1.00 | 47.56 |
| ATOM | 10124 | C | LEU | 5269 | 41.403 | 33.344 | 19.049 | 1.00 | 47.65 |
| ATOM | 10125 | O | LEU | 5269 | 40.623 | 34.280 | 18.836 | 1.00 | 47.78 |
| ATOM | 10126 | N | GLY | 5270 | 42.456 | 33.455 | 19.856 | 1.00 | 47.85 |
| ATOM | 10127 | CA | GLY | 5270 | 42.749 | 34.718 | 20.510 | 1.00 | 48.22 |
| ATOM | 10128 | C | GLY | 5270 | 41.959 | 35.004 | 21.772 | 1.00 | 48.24 |
| ATOM | 10129 | O | GLY | 5270 | 42.139 | 36.055 | 22.395 | 1.00 | 48.44 |
| ATOM | 10130 | N | SER | 5271 | 41.084 | 34.082 | 22.154 | 1.00 | 47.74 |
| ATOM | 10131 | CA | SER | 5271 | 40.289 | 34.262 | 23.359 | 1.00 | 47.60 |
| ATOM | 10132 | CB | SER | 5271 | 39.096 | 33.317 | 23.337 | 1.00 | 47.37 |
| ATOM | 10133 | OG | SER | 5271 | 38.342 | 33.505 | 22.155 | 1.00 | 48.08 |
| ATOM | 10134 | C | SER | 5271 | 41.127 | 33.988 | 24.602 | 1.00 | 47.36 |
| ATOM | 10135 | O | SER | 5271 | 42.253 | 33.492 | 24.515 | 1.00 | 47.33 |
| ATOM | 10136 | N | ASN | 5272 | 40.576 | 34.331 | 25.757 | 1.00 | 46.83 |
| ATOM | 10137 | CA | ASN | 5272 | 41.247 | 34.080 | 27.013 | 1.00 | 46.38 |
| ATOM | 10138 | CB | ASN | 5272 | 41.318 | 35.359 | 27.833 | 1.00 | 46.54 |
| ATOM | 10139 | CG | ASN | 5272 | 42.106 | 36.450 | 27.125 | 1.00 | 47.02 |
| ATOM | 10140 | OD1 | ASN | 5272 | 43.240 | 36.232 | 26.691 | 1.00 | 47.08 |
| ATOM | 10141 | ND2 | ASN | 5272 | 41.507 | 37.628 | 26.999 | 1.00 | 46.67 |
| ATOM | 10142 | C | ASN | 5272 | 40.381 | 33.024 | 27.672 | 1.00 | 46.10 |
| ATOM | 10143 | O | ASN | 5272 | 39.157 | 33.086 | 27.601 | 1.00 | 45.92 |
| ATOM | 10144 | N | VAL | 5273 | 41.010 | 32.033 | 28.285 | 1.00 | 45.60 |
| ATOM | 10145 | CA | VAL | 5273 | 40.256 | 30.960 | 28.906 | 1.00 | 44.42 |
| ATOM | 10146 | CB | VAL | 5273 | 40.208 | 29.732 | 27.965 | 1.00 | 44.17 |
| ATOM | 10147 | CG1 | VAL | 5273 | 41.607 | 29.380 | 27.513 | 1.00 | 43.76 |
| ATOM | 10148 | CG2 | VAL | 5273 | 39.583 | 28.548 | 28.673 | 1.00 | 44.17 |
| ATOM | 10149 | C | VAL | 5273 | 40.846 | 30.551 | 30.240 | 1.00 | 43.72 |
| ATOM | 10150 | O | VAL | 5273 | 42.060 | 30.616 | 30.442 | 1.00 | 43.69 |
| ATOM | 10151 | N | GLU | 5274 | 39.979 | 30.139 | 31.155 | 1.00 | 42.65 |
| ATOM | 10152 | CA | GLU | 5274 | 40.438 | 29.706 | 32.456 | 1.00 | 42.01 |
| ATOM | 10153 | CB | GLU | 5274 | 40.140 | 30.755 | 33.521 | 1.00 | 42.19 |
| ATOM | 10154 | CG | GLU | 5274 | 38.697 | 30.839 | 33.941 | 1.00 | 42.83 |
| ATOM | 10155 | CD | GLU | 5274 | 38.542 | 31.556 | 35.264 | 1.00 | 43.94 |
| ATOM | 10156 | OE1 | GLU | 5274 | 39.049 | 32.694 | 35.383 | 1.00 | 44.14 |
| ATOM | 10157 | OE2 | GLU | 5274 | 37.919 | 30.982 | 36.185 | 1.00 | 44.10 |
| ATOM | 10158 | C | GLU | 5274 | 39.819 | 28.385 | 32.875 | 1.00 | 41.03 |
| ATOM | 10159 | O | GLU | 5274 | 38.599 | 28.236 | 32.927 | 1.00 | 40.80 |
| ATOM | 10160 | N | PHE | 5275 | 40.678 | 27.419 | 33.158 | 1.00 | 39.45 |
| ATOM | 10161 | CA | PHE | 5275 | 40.223 | 26.121 | 33.610 | 1.00 | 37.92 |
| ATOM | 10162 | CB | PHE | 5275 | 41.163 | 25.019 | 33.123 | 1.00 | 36.59 |
| ATOM | 10163 | CG | PHE | 5275 | 40.998 | 24.667 | 31.666 | 1.00 | 35.45 |
| ATOM | 10164 | CD1 | PHE | 5275 | 39.869 | 23.982 | 31.225 | 1.00 | 34.73 |
| ATOM | 10165 | CD2 | PHE | 5275 | 41.986 | 24.991 | 30.741 | 1.00 | 34.12 |
| ATOM | 10166 | CE1 | PHE | 5275 | 39.728 | 23.621 | 29.891 | 1.00 | 34.58 |
| ATOM | 10167 | CE2 | PHE | 5275 | 41.855 | 24.637 | 29.405 | 1.00 | 34.27 |
| ATOM | 10168 | CZ | PHE | 5275 | 40.726 | 23.949 | 28.975 | 1.00 | 34.30 |
| ATOM | 10169 | C | PHE | 5275 | 40.215 | 26.157 | 35.130 | 1.00 | 37.71 |
| ATOM | 10170 | O | PHE | 5275 | 41.066 | 26.790 | 35.751 | 1.00 | 36.89 |
| ATOM | 10171 | N | MSE | 5276 | 39.243 | 25.485 | 35.729 | 1.00 | 37.93 |
| ATOM | 10172 | CA | MSE | 5276 | 39.150 | 25.453 | 37.175 | 1.00 | 38.01 |
| ATOM | 10173 | CB | MSE | 5276 | 37.818 | 26.030 | 37.646 | 1.00 | 39.20 |
| ATOM | 10174 | CG | MSE | 5276 | 37.762 | 27.553 | 37.711 | 1.00 | 41.11 |
| ATOM | 10175 | SE | MSE | 5276 | 36.103 | 28.108 | 38.178 | 1.00 | 44.10 |
| ATOM | 10176 | CE | MSE | 5276 | 36.170 | 27.905 | 39.937 | 1.00 | 41.39 |
| ATOM | 10177 | C | MSE | 5276 | 39.290 | 24.052 | 37.711 | 1.00 | 37.40 |
| ATOM | 10178 | O | MSE | 5276 | 39.121 | 23.076 | 36.986 | 1.00 | 36.63 |
| ATOM | 10179 | N | CYS | 5277 | 39.607 | 23.970 | 38.996 | 1.00 | 37.24 |
| ATOM | 10180 | CA | CYS | 5277 | 39.755 | 22.698 | 39.671 | 1.00 | 37.32 |
| ATOM | 10181 | CB | CYS | 5277 | 41.119 | 22.101 | 39.400 | 1.00 | 38.15 |
| ATOM | 10182 | SG | CYS | 5277 | 41.161 | 20.398 | 39.934 | 1.00 | 42.84 |
| ATOM | 10183 | C | CYS | 5277 | 39.580 | 22.844 | 41.173 | 1.00 | 36.48 |
| ATOM | 10184 | O | CYS | 5277 | 40.347 | 23.541 | 41.824 | 1.00 | 36.62 |
| ATOM | 10185 | N | LYS | 5278 | 38.574 | 22.173 | 41.718 | 1.00 | 35.82 |
| ATOM | 10186 | CA | LYS | 5278 | 38.300 | 22.230 | 43.145 | 1.00 | 34.78 |
| ATOM | 10187 | CB | LYS | 5278 | 36.806 | 22.463 | 43.378 | 1.00 | 35.49 |
| ATOM | 10188 | CG | LYS | 5278 | 36.377 | 23.881 | 43.005 | 1.00 | 36.59 |
| ATOM | 10189 | CD | LYS | 5278 | 37.176 | 24.876 | 43.827 | 1.00 | 38.50 |
| ATOM | 10190 | CE | LYS | 5278 | 36.787 | 26.317 | 43.556 | 1.00 | 39.81 |
| ATOM | 10191 | NZ | LYS | 5278 | 37.376 | 27.213 | 44.608 | 1.00 | 40.66 |
| ATOM | 10192 | C | LYS | 5278 | 38.784 | 20.961 | 43.834 | 1.00 | 33.20 |
| ATOM | 10193 | O | LYS | 5278 | 38.314 | 19.856 | 43.565 | 1.00 | 33.22 |
| ATOM | 10194 | N | VAL | 5279 | 39.737 | 21.136 | 44.734 | 1.00 | 30.93 |
| ATOM | 10195 | CA | VAL | 5279 | 40.320 | 20.010 | 45.430 | 1.00 | 28.74 |
| ATOM | 10196 | CB | VAL | 5279 | 41.847 | 20.014 | 45.280 | 1.00 | 28.11 |
| ATOM | 10197 | CG1 | VAL | 5279 | 42.457 | 18.860 | 46.035 | 1.00 | 26.46 |
| ATOM | 10198 | CG2 | VAL | 5279 | 42.210 | 19.951 | 43.818 | 1.00 | 27.73 |
| ATOM | 10199 | C | VAL | 5279 | 40.012 | 19.939 | 46.904 | 1.00 | 28.36 |
| ATOM | 10200 | O | VAL | 5279 | 39.953 | 20.947 | 47.601 | 1.00 | 28.06 |
| ATOM | 10201 | N | TYR | 5280 | 39.807 | 18.722 | 47.377 | 1.00 | 28.13 |
| ATOM | 10202 | CA | TYR | 5280 | 39.569 | 18.504 | 48.786 | 1.00 | 27.39 |
| ATOM | 10203 | CB | TYR | 5280 | 38.164 | 17.968 | 49.070 | 1.00 | 26.36 |
| ATOM | 10204 | CG | TYR | 5280 | 38.041 | 17.600 | 50.527 | 1.00 | 26.25 |
| ATOM | 10205 | CD1 | TYR | 5280 | 37.944 | 18.589 | 51.502 | 1.00 | 26.23 |
| ATOM | 10206 | CE1 | TYR | 5280 | 38.018 | 18.277 | 52.856 | 1.00 | 27.10 |
| ATOM | 10207 | CD2 | TYR | 5280 | 38.188 | 16.275 | 50.946 | 1.00 | 26.20 |
| ATOM | 10208 | CE2 | TYR | 5280 | 38.263 | 15.947 | 52.290 | 1.00 | 26.49 |
| ATOM | 10209 | CZ | TYR | 5280 | 38.186 | 16.951 | 53.244 | 1.00 | 27.36 |
| ATOM | 10210 | OH | TYR | 5280 | 38.337 | 16.650 | 54.582 | 1.00 | 27.42 |
| ATOM | 10211 | C | TYR | 5280 | 40.593 | 17.482 | 49.273 | 1.00 | 26.90 |
| ATOM | 10212 | O | TYR | 5280 | 40.830 | 16.467 | 48.623 | 1.00 | 26.28 |
| ATOM | 10213 | N | SER | 5281 | 41.202 | 17.763 | 50.415 | 1.00 | 26.83 |
| ATOM | 10214 | CA | SER | 5281 | 42.176 | 16.860 | 50.999 | 1.00 | 26.77 |
| ATOM | 10215 | CB | SER | 5281 | 43.473 | 16.836 | 50.187 | 1.00 | 27.14 |
| ATOM | 10216 | OG | SER | 5281 | 44.498 | 16.135 | 50.889 | 1.00 | 26.75 |
| ATOM | 10217 | C | SER | 5281 | 42.483 | 17.302 | 52.414 | 1.00 | 26.84 |
| ATOM | 10218 | O | SER | 5281 | 42.708 | 18.488 | 52.664 | 1.00 | 27.00 |
| ATOM | 10219 | N | ASP | 5282 | 42.475 | 16.347 | 53.343 | 1.00 | 27.34 |
| ATOM | 10220 | CA | ASP | 5282 | 42.790 | 16.644 | 54.738 | 1.00 | 27.40 |
| ATOM | 10221 | CB | ASP | 5282 | 42.425 | 15.471 | 55.642 | 1.00 | 27.94 |
| ATOM | 10222 | CG | ASP | 5282 | 42.603 | 15.790 | 57.116 | 1.00 | 29.06 |
| ATOM | 10223 | OD1 | ASP | 5282 | 42.118 | 14.988 | 57.939 | 1.00 | 30.66 |
| ATOM | 10224 | OD2 | ASP | 5282 | 43.226 | 16.822 | 57.459 | 1.00 | 27.86 |
| ATOM | 10225 | C | ASP | 5282 | 44.285 | 16.934 | 54.785 | 1.00 | 27.07 |
| ATOM | 10226 | O | ASP | 5282 | 44.697 | 18.031 | 55.150 | 1.00 | 26.29 |
| ATOM | 10227 | N | PRO | 5283 | 45.121 | 15.953 | 54.438 | 1.00 | 27.13 |
| ATOM | 10228 | CD | PRO | 5283 | 44.917 | 14.520 | 54.176 | 1.00 | 27.06 |
| ATOM | 10229 | CA | PRO | 5283 | 46.548 | 16.291 | 54.473 | 1.00 | 27.68 |
| ATOM | 10230 | CB | PRO | 5283 | 47.233 | 14.959 | 54.166 | 1.00 | 27.56 |
| ATOM | 10231 | CG | PRO | 5283 | 46.226 | 13.935 | 54.627 | 1.00 | 28.45 |
| ATOM | 10232 | C | PRO | 5283 | 46.751 | 17.311 | 53.332 | 1.00 | 27.92 |
| ATOM | 10233 | O | PRO | 5283 | 46.039 | 17.277 | 52.314 | 1.00 | 27.61 |
| ATOM | 10234 | N | GLN | 5284 | 47.705 | 18.216 | 53.502 | 1.00 | 28.03 |
| ATOM | 10235 | CA | GLN | 5284 | 47.994 | 19.238 | 52.496 | 1.00 | 27.94 |
| ATOM | 10236 | CB | GLN | 5284 | 49.225 | 20.027 | 52.939 | 1.00 | 27.20 |
| ATOM | 10237 | CG | GLN | 5284 | 49.090 | 21.522 | 52.814 | 1.00 | 28.39 |
| ATOM | 10238 | CD | GLN | 5284 | 47.813 | 22.049 | 53.422 | 1.00 | 26.91 |
| ATOM | 10239 | OE1 | GLN | 5284 | 47.514 | 21.802 | 54.589 | 1.00 | 26.94 |
| ATOM | 10240 | NE2 | GLN | 5284 | 47.054 | 22.785 | 52.633 | 1.00 | 27.19 |
| ATOM | 10241 | C | GLN | 5284 | 48.236 | 18.597 | 51.122 | 1.00 | 27.97 |
| ATOM | 10242 | O | GLN | 5284 | 49.077 | 17.720 | 50.976 | 1.00 | 28.43 |
| ATOM | 10243 | N | PRO | 5285 | 47.478 | 19.011 | 50.099 | 1.00 | 28.19 |
| ATOM | 10244 | CD | PRO | 5285 | 46.213 | 19.770 | 50.153 | 1.00 | 27.10 |
| ATOM | 10245 | CA | PRO | 5285 | 47.665 | 18.436 | 48.761 | 1.00 | 28.12 |
| ATOM | 10246 | CB | PRO | 5285 | 46.259 | 18.444 | 48.195 | 1.00 | 26.73 |
| ATOM | 10247 | CG | PRO | 5285 | 45.761 | 19.766 | 48.699 | 1.00 | 25.67 |
| ATOM | 10248 | C | PRO | 5285 | 48.599 | 19.246 | 47.868 | 1.00 | 28.36 |
| ATOM | 10249 | O | PRO | 5285 | 48.724 | 20.467 | 48.007 | 1.00 | 28.38 |
| ATOM | 10250 | N | HIS | 5286 | 49.248 | 18.565 | 46.937 | 1.00 | 28.59 |
| ATOM | 10251 | CA | HIS | 5286 | 50.121 | 19.256 | 46.005 | 1.00 | 28.71 |
| ATOM | 10252 | CB | HIS | 5286 | 51.520 | 18.618 | 45.930 | 1.00 | 28.72 |
| ATOM | 10253 | CG | HIS | 5286 | 52.451 | 19.330 | 44.993 | 1.00 | 29.56 |
| ATOM | 10254 | CD2 | HIS | 5286 | 53.154 | 20.481 | 45.138 | 1.00 | 29.69 |
| ATOM | 10255 | ND1 | HIS | 5286 | 52.668 | 18.914 | 43.695 | 1.00 | 30.63 |
| ATOM | 10256 | CE1 | HIS | 5286 | 53.459 | 19.780 | 43.083 | 1.00 | 30.16 |
| ATOM | 10257 | NE2 | HIS | 5286 | 53.767 | 20.739 | 43.936 | 1.00 | 29.14 |
| ATOM | 10258 | C | HIS | 5286 | 49.458 | 19.203 | 44.646 | 1.00 | 28.24 |
| ATOM | 10259 | O | HIS | 5286 | 49.323 | 18.135 | 44.068 | 1.00 | 28.10 |
| ATOM | 10260 | N | ILE | 5287 | 49.040 | 20.366 | 44.155 | 1.00 | 29.06 |
| ATOM | 10261 | CA | ILE | 5287 | 48.373 | 20.477 | 42.862 | 1.00 | 29.58 |
| ATOM | 10262 | CB | ILE | 5287 | 47.296 | 21.563 | 42.862 | 1.00 | 29.09 |
| ATOM | 10263 | CG2 | ILE | 5287 | 46.590 | 21.596 | 41.512 | 1.00 | 28.74 |
| ATOM | 10264 | CG1 | ILE | 5287 | 46.251 | 21.279 | 43.933 | 1.00 | 29.17 |
| ATOM | 10265 | CD1 | ILE | 5287 | 45.146 | 22.333 | 43.942 | 1.00 | 29.77 |
| ATOM | 10266 | C | ILE | 5287 | 49.352 | 20.813 | 41.753 | 1.00 | 30.18 |
| ATOM | 10267 | O | ILE | 5287 | 50.379 | 21.432 | 41.991 | 1.00 | 30.78 |
| ATOM | 10268 | N | GLN | 5288 | 49.004 | 20.424 | 40.532 | 1.00 | 31.03 |
| ATOM | 10269 | CA | GLN | 5288 | 49.857 | 20.646 | 39.379 | 1.00 | 31.32 |
| ATOM | 10270 | CB | GLN | 5288 | 50.859 | 19.503 | 39.316 | 1.00 | 32.32 |
| ATOM | 10271 | CG | GLN | 5288 | 51.961 | 19.597 | 38.287 | 1.00 | 34.08 |
| ATOM | 10272 | CD | GLN | 5288 | 52.831 | 18.348 | 38.314 | 1.00 | 34.59 |
| ATOM | 10273 | OE1 | GLN | 5288 | 53.236 | 17.890 | 39.385 | 1.00 | 34.67 |
| ATOM | 10274 | NE2 | GLN | 5288 | 53.112 | 17.786 | 37.139 | 1.00 | 35.19 |
| ATOM | 10275 | C | GLN | 5288 | 48.984 | 20.647 | 38.139 | 1.00 | 31.26 |
| ATOM | 10276 | O | GLN | 5288 | 48.059 | 19.849 | 38.048 | 1.00 | 32.80 |
| ATOM | 10277 | N | TRP | 5289 | 49.253 | 21.553 | 37.203 | 1.00 | 30.40 |
| ATOM | 10278 | CA | TRP | 5289 | 48.494 | 21.620 | 35.957 | 1.00 | 29.49 |
| ATOM | 10279 | CB | TRP | 5289 | 47.994 | 23.040 | 35.693 | 1.00 | 26.55 |
| ATOM | 10280 | CG | TRP | 5289 | 46.796 | 23.457 | 36.513 | 1.00 | 23.73 |
| ATOM | 10281 | CD2 | TRP | 5289 | 45.420 | 23.306 | 36.154 | 1.00 | 21.76 |
| ATOM | 10282 | CE2 | TRP | 5289 | 44.650 | 23.853 | 37.206 | 1.00 | 21.39 |
| ATOM | 10283 | CE3 | TRP | 5289 | 44.761 | 22.761 | 35.044 | 1.00 | 21.26 |
| ATOM | 10284 | CD1 | TRP | 5289 | 46.805 | 24.067 | 37.734 | 1.00 | 22.50 |
| ATOM | 10285 | NE1 | TRP | 5289 | 45.521 | 24.311 | 38.154 | 1.00 | 20.90 |
| ATOM | 10286 | CZ2 | TRP | 5289 | 43.248 | 23.871 | 37.183 | 1.00 | 21.66 |
| ATOM | 10287 | CZ3 | TRP | 5289 | 43.368 | 22.776 | 35.018 | 1.00 | 21.98 |
| ATOM | 10288 | CH2 | TRP | 5289 | 42.625 | 23.329 | 36.085 | 1.00 | 21.49 |
| ATOM | 10289 | C | TRP | 5289 | 49.370 | 21.170 | 34.790 | 1.00 | 31.04 |
| ATOM | 10290 | O | TRP | 5289 | 50.483 | 21.659 | 34.616 | 1.00 | 31.38 |
| ATOM | 10291 | N | LEU | 5290 | 48.863 | 20.242 | 33.989 | 1.00 | 32.31 |
| ATOM | 10292 | CA | LEU | 5290 | 49.612 | 19.718 | 32.853 | 1.00 | 34.36 |
| ATOM | 10293 | CB | LEU | 5290 | 49.869 | 18.219 | 33.018 | 1.00 | 35.64 |
| ATOM | 10294 | CG | LEU | 5290 | 50.528 | 17.732 | 34.299 | 1.00 | 37.42 |
| ATOM | 10295 | CD1 | LEU | 5290 | 49.573 | 17.941 | 35.467 | 1.00 | 38.46 |
| ATOM | 10296 | CD2 | LEU | 5290 | 50.871 | 16.267 | 34.162 | 1.00 | 38.38 |
| ATOM | 10297 | C | LEU | 5290 | 48.880 | 19.902 | 31.540 | 1.00 | 35.25 |
| ATOM | 10298 | O | LEU | 5290 | 47.654 | 19.962 | 31.504 | 1.00 | 35.01 |
| ATOM | 10299 | N | LYS | 5291 | 49.649 | 19.967 | 30.460 | 1.00 | 36.81 |
| ATOM | 10300 | CA | LYS | 5291 | 49.107 | 20.100 | 29.109 | 1.00 | 37.80 |
| ATOM | 10301 | CB | LYS | 5291 | 49.613 | 21.396 | 28.470 | 1.00 | 39.53 |
| ATOM | 10302 | CG | LYS | 5291 | 48.890 | 21.879 | 27.202 | 1.00 | 41.79 |
| ATOM | 10303 | CD | LYS | 5291 | 48.912 | 20.863 | 26.075 | 1.00 | 44.34 |
| ATOM | 10304 | CE | LYS | 5291 | 48.428 | 21.464 | 24.738 | 1.00 | 45.53 |
| ATOM | 10305 | NZ | LYS | 5291 | 49.435 | 22.400 | 24.102 | 1.00 | 45.52 |
| ATOM | 10306 | C | LYS | 5291 | 49.670 | 18.900 | 28.360 | 1.00 | 38.13 |
| ATOM | 10307 | O | LYS | 5291 | 50.879 | 18.686 | 28.356 | 1.00 | 38.68 |
| ATOM | 10308 | N | HIS | 5292 | 48.804 | 18.092 | 27.762 | 1.00 | 38.93 |
| ATOM | 10309 | CA | HIS | 5292 | 49.265 | 16.926 | 27.011 | 1.00 | 39.49 |
| ATOM | 10310 | CB | HIS | 5292 | 48.121 | 15.943 | 26.802 | 1.00 | 39.59 |
| ATOM | 10311 | CG | HIS | 5292 | 47.678 | 15.275 | 28.060 | 1.00 | 40.09 |
| ATOM | 10312 | CD2 | HIS | 5292 | 47.320 | 15.787 | 29.261 | 1.00 | 40.51 |
| ATOM | 10313 | ND1 | HIS | 5292 | 47.631 | 13.906 | 28.195 | 1.00 | 39.83 |
| ATOM | 10314 | CE1 | HIS | 5292 | 47.269 | 13.602 | 29.428 | 1.00 | 40.50 |
| ATOM | 10315 | NE2 | HIS | 5292 | 47.075 | 14.725 | 30.095 | 1.00 | 40.90 |
| ATOM | 10316 | C | HIS | 5292 | 49.785 | 17.414 | 25.667 | 1.00 | 39.74 |
| ATOM | 10317 | O | HIS | 5292 | 49.063 | 18.081 | 24.936 | 1.00 | 39.97 |
| ATOM | 10318 | N | ILE | 5293 | 51.029 | 17.102 | 25.326 | 1.00 | 38.77 |
| ATOM | 10319 | CA | ILE | 5293 | 51.562 | 17.591 | 24.065 | 1.00 | 38.31 |
| ATOM | 10320 | CB | ILE | 5293 | 52.765 | 18.544 | 24.300 | 1.00 | 37.17 |
| ATOM | 10321 | CG2 | ILE | 5293 | 52.316 | 19.752 | 25.117 | 1.00 | 35.55 |
| ATOM | 10322 | CG1 | ILE | 5293 | 53.907 | 17.801 | 25.012 | 1.00 | 36.13 |
| ATOM | 10323 | CD1 | ILE | 5293 | 55.161 | 18.630 | 25.201 | 1.00 | 33.80 |
| ATOM | 10324 | C | ILE | 5293 | 51.977 | 16.513 | 23.084 | 1.00 | 39.11 |
| ATOM | 10325 | O | ILE | 5293 | 51.998 | 15.328 | 23.412 | 1.00 | 39.35 |
| ATOM | 10326 | N | GLU | 5294 | 52.286 | 16.941 | 21.867 | 1.00 | 40.36 |
| ATOM | 10327 | CA | GLU | 5294 | 52.729 | 16.039 | 20.813 | 1.00 | 41.99 |
| ATOM | 10328 | CB | GLU | 5294 | 51.870 | 16.208 | 19.552 | 1.00 | 40.99 |
| ATOM | 10329 | C | GLU | 5294 | 54.185 | 16.367 | 20.487 | 1.00 | 43.41 |
| ATOM | 10330 | O | GLU | 5294 | 54.501 | 17.472 | 20.039 | 1.00 | 43.12 |
| ATOM | 10331 | N | VAL | 5295 | 55.072 | 15.416 | 20.742 | 1.00 | 45.38 |
| ATOM | 10332 | CA | VAL | 5295 | 56.482 | 15.608 | 20.438 | 1.00 | 47.83 |
| ATOM | 10333 | CB | VAL | 5295 | 57.368 | 15.067 | 21.571 | 1.00 | 47.77 |
| ATOM | 10334 | CG1 | VAL | 5295 | 58.811 | 15.001 | 21.119 | 1.00 | 47.71 |
| ATOM | 10335 | CG2 | VAL | 5295 | 57.243 | 15.975 | 22.781 | 1.00 | 47.62 |
| ATOM | 10336 | C | VAL | 5295 | 56.782 | 14.869 | 19.137 | 1.00 | 49.26 |
| ATOM | 10337 | O | VAL | 5295 | 56.832 | 13.639 | 19.105 | 1.00 | 49.49 |
| ATOM | 10338 | N | ASN | 5296 | 56.973 | 15.625 | 18.063 | 1.00 | 51.19 |
| ATOM | 10339 | CA | ASN | 5296 | 57.221 | 15.022 | 16.759 | 1.00 | 53.69 |
| ATOM | 10340 | CB | ASN | 5296 | 58.476 | 14.144 | 16.787 | 1.00 | 55.05 |
| ATOM | 10341 | CG | ASN | 5296 | 59.733 | 14.932 | 17.095 | 1.00 | 56.48 |
| ATOM | 10342 | OD1 | ASN | 5296 | 59.955 | 16.012 | 16.539 | 1.00 | 57.11 |
| ATOM | 10343 | ND2 | ASN | 5296 | 60.572 | 14.389 | 17.974 | 1.00 | 56.57 |
| ATOM | 10344 | C | ASN | 5296 | 56.015 | 14.167 | 16.361 | 1.00 | 54.68 |
| ATOM | 10345 | O | ASN | 5296 | 56.099 | 12.934 | 16.302 | 1.00 | 54.21 |
| ATOM | 10346 | N | GLY | 5297 | 54.890 | 14.835 | 16.118 | 1.00 | 55.53 |
| ATOM | 10347 | CA | GLY | 5297 | 53.677 | 14.151 | 15.703 | 1.00 | 56.63 |
| ATOM | 10348 | C | GLY | 5297 | 53.045 | 13.148 | 16.653 | 1.00 | 57.60 |
| ATOM | 10349 | O | GLY | 5297 | 51.843 | 12.888 | 16.558 | 1.00 | 57.89 |
| ATOM | 10350 | N | SER | 5298 | 53.826 | 12.581 | 17.567 | 1.00 | 58.23 |
| ATOM | 10351 | CA | SER | 5298 | 53.281 | 11.597 | 18.496 | 1.00 | 58.64 |
| ATOM | 10352 | CB | SER | 5298 | 54.249 | 10.435 | 18.642 | 1.00 | 58.50 |
| ATOM | 10353 | OG | SER | 5298 | 55.514 | 10.919 | 19.031 | 1.00 | 59.49 |
| ATOM | 10354 | C | SER | 5298 | 52.956 | 12.165 | 19.872 | 1.00 | 58.82 |
| ATOM | 10355 | O | SER | 5298 | 53.648 | 13.049 | 20.380 | 1.00 | 58.64 |
| ATOM | 10356 | N | LYS | 5299 | 51.893 | 11.632 | 20.466 | 1.00 | 59.07 |
| ATOM | 10357 | CA | LYS | 5299 | 51.428 | 12.055 | 21.779 | 1.00 | 59.23 |
| ATOM | 10358 | CB | LYS | 5299 | 49.899 | 11.940 | 21.867 | 1.00 | 59.15 |
| ATOM | 10359 | CG | LYS | 5299 | 49.119 | 12.608 | 20.747 | 1.00 | 59.36 |
| ATOM | 10360 | CD | LYS | 5299 | 47.627 | 12.297 | 20.874 | 1.00 | 59.78 |
| ATOM | 10361 | CE | LYS | 5299 | 46.819 | 12.808 | 19.669 | 1.00 | 60.26 |
| ATOM | 10362 | NZ | LYS | 5299 | 45.348 | 12.490 | 19.752 | 1.00 | 59.35 |
| ATOM | 10363 | C | LYS | 5299 | 52.041 | 11.172 | 22.861 | 1.00 | 59.33 |
| ATOM | 10364 | O | LYS | 5299 | 52.012 | 11.513 | 24.045 | 1.00 | 59.23 |
| ATOM | 10365 | N | ILE | 5300 | 52.598 | 10.038 | 22.448 | 1.00 | 59.52 |
| ATOM | 10366 | CA | ILE | 5300 | 53.174 | 9.096 | 23.396 | 1.00 | 59.85 |
| ATOM | 10367 | CB | ILE | 5300 | 52.599 | 7.685 | 23.167 | 1.00 | 59.82 |
| ATOM | 10368 | CG2 | ILE | 5300 | 53.043 | 6.755 | 24.282 | 1.00 | 59.60 |
| ATOM | 10369 | CG1 | ILE | 5300 | 51.072 | 7.747 | 23.116 | 1.00 | 59.87 |
| ATOM | 10370 | CD1 | ILE | 5300 | 50.437 | 8.292 | 24.378 | 1.00 | 60.49 |
| ATOM | 10371 | C | ILE | 5300 | 54.697 | 8.995 | 23.390 | 1.00 | 60.27 |
| ATOM | 10372 | O | ILE | 5300 | 55.323 | 8.829 | 22.344 | 1.00 | 59.86 |
| ATOM | 10373 | N | GLY | 5301 | 55.273 | 9.090 | 24.586 | 1.00 | 60.96 |
| ATOM | 10374 | CA | GLY | 5301 | 56.710 | 8.988 | 24.752 | 1.00 | 61.89 |
| ATOM | 10375 | C | GLY | 5301 | 57.122 | 7.533 | 24.887 | 1.00 | 62.39 |
| ATOM | 10376 | O | GLY | 5301 | 56.284 | 6.682 | 25.190 | 1.00 | 62.69 |
| ATOM | 10377 | N | PRO | 5302 | 58.411 | 7.217 | 24.685 | 1.00 | 62.57 |
| ATOM | 10378 | CD | PRO | 5302 | 59.472 | 8.224 | 24.502 | 1.00 | 62.72 |
| ATOM | 10379 | CA | PRO | 5302 | 58.991 | 5.867 | 24.765 | 1.00 | 62.69 |
| ATOM | 10380 | CB | PRO | 5302 | 60.483 | 6.155 | 24.921 | 1.00 | 62.80 |
| ATOM | 10381 | CG | PRO | 5302 | 60.656 | 7.380 | 24.074 | 1.00 | 62.86 |
| ATOM | 10382 | C | PRO | 5302 | 58.447 | 4.950 | 25.873 | 1.00 | 62.40 |
| ATOM | 10383 | O | PRO | 5302 | 58.164 | 3.776 | 25.637 | 1.00 | 62.11 |
| ATOM | 10384 | N | ASP | 5303 | 58.316 | 5.486 | 27.080 | 1.00 | 62.03 |
| ATOM | 10385 | CA | ASP | 5303 | 57.811 | 4.721 | 28.213 | 1.00 | 61.87 |
| ATOM | 10386 | CB | ASP | 5303 | 58.035 | 5.516 | 29.496 | 1.00 | 62.94 |
| ATOM | 10387 | CG | ASP | 5303 | 57.492 | 6.939 | 29.400 | 1.00 | 63.81 |
| ATOM | 10388 | OD1 | ASP | 5303 | 57.562 | 7.669 | 30.411 | 1.00 | 64.46 |
| ATOM | 10389 | OD2 | ASP | 5303 | 56.999 | 7.327 | 28.315 | 1.00 | 63.83 |
| ATOM | 10390 | C | ASP | 5303 | 56.319 | 4.392 | 28.083 | 1.00 | 61.35 |
| ATOM | 10391 | O | ASP | 5303 | 55.693 | 3.947 | 29.049 | 1.00 | 61.31 |
| ATOM | 10392 | N | ASN | 5304 | 55.761 | 4.609 | 26.892 | 1.00 | 59.99 |
| ATOM | 10393 | CA | ASN | 5304 | 54.343 | 4.361 | 26.622 | 1.00 | 58.57 |
| ATOM | 10394 | CB | ASN | 5304 | 53.974 | 2.900 | 26.938 | 1.00 | 59.52 |
| ATOM | 10395 | CG | ASN | 5304 | 52.581 | 2.511 | 26.419 | 1.00 | 59.85 |
| ATOM | 10396 | OD1 | ASN | 5304 | 52.254 | 2.738 | 25.253 | 1.00 | 60.05 |
| ATOM | 10397 | ND2 | ASN | 5304 | 51.767 | 1.909 | 27.286 | 1.00 | 59.59 |
| ATOM | 10398 | C | ASN | 5304 | 53.468 | 5.323 | 27.434 | 1.00 | 57.22 |
| ATOM | 10399 | O | ASN | 5304 | 52.258 | 5.128 | 27.560 | 1.00 | 56.75 |
| ATOM | 10400 | N | LEU | 5305 | 54.101 | 6.355 | 27.988 | 1.00 | 55.89 |
| ATOM | 10401 | CA | LEU | 5305 | 53.408 | 7.378 | 28.769 | 1.00 | 54.56 |
| ATOM | 10402 | CB | LEU | 5305 | 54.242 | 7.835 | 29.967 | 1.00 | 55.05 |
| ATOM | 10403 | CG | LEU | 5305 | 54.198 | 7.051 | 31.276 | 1.00 | 55.37 |
| ATOM | 10404 | CD1 | LEU | 5305 | 52.760 | 6.937 | 31.740 | 1.00 | 54.99 |
| ATOM | 10405 | CD2 | LEU | 5305 | 54.818 | 5.681 | 31.088 | 1.00 | 55.86 |
| ATOM | 10406 | C | LEU | 5305 | 53.203 | 8.571 | 27.863 | 1.00 | 53.34 |
| ATOM | 10407 | O | LEU | 5305 | 53.934 | 8.745 | 26.891 | 1.00 | 53.64 |
| ATOM | 10408 | N | PRO | 5306 | 52.198 | 9.407 | 28.162 | 1.00 | 52.05 |
| ATOM | 10409 | CD | PRO | 5306 | 51.063 | 9.121 | 29.059 | 1.00 | 51.32 |
| ATOM | 10410 | CA | PRO | 5306 | 51.931 | 10.592 | 27.340 | 1.00 | 50.63 |
| ATOM | 10411 | CB | PRO | 5306 | 50.455 | 10.873 | 27.618 | 1.00 | 50.67 |
| ATOM | 10412 | CG | PRO | 5306 | 50.295 | 10.418 | 29.032 | 1.00 | 50.84 |
| ATOM | 10413 | C | PRO | 5306 | 52.825 | 11.785 | 27.674 | 1.00 | 49.17 |
| ATOM | 10414 | O | PRO | 5306 | 53.087 | 12.074 | 28.844 | 1.00 | 48.51 |
| ATOM | 10415 | N | TYR | 5307 | 53.298 | 12.467 | 26.637 | 1.00 | 48.14 |
| ATOM | 10416 | CA | TYR | 5307 | 54.141 | 13.642 | 26.817 | 1.00 | 47.11 |
| ATOM | 10417 | CB | TYR | 5307 | 54.611 | 14.192 | 25.471 | 1.00 | 47.60 |
| ATOM | 10418 | CG | TYR | 5307 | 55.587 | 13.322 | 24.728 | 1.00 | 49.25 |
| ATOM | 10419 | CD1 | TYR | 5307 | 55.292 | 12.850 | 23.444 | 1.00 | 49.89 |
| ATOM | 10420 | CE1 | TYR | 5307 | 56.208 | 12.076 | 22.735 | 1.00 | 50.56 |
| ATOM | 10421 | CD2 | TYR | 5307 | 56.827 | 12.995 | 25.287 | 1.00 | 49.53 |
| ATOM | 10422 | CE2 | TYR | 5307 | 57.752 | 12.221 | 24.586 | 1.00 | 49.84 |
| ATOM | 10423 | CZ | TYR | 5307 | 57.437 | 11.766 | 23.314 | 1.00 | 50.77 |
| ATOM | 10424 | OH | TYR | 5307 | 58.345 | 10.999 | 22.620 | 1.00 | 51.70 |
| ATOM | 10425 | C | TYR | 5307 | 53.304 | 14.708 | 27.498 | 1.00 | 46.11 |
| ATOM | 10426 | O | TYR | 5307 | 52.194 | 15.003 | 27.043 | 1.00 | 46.08 |
| ATOM | 10427 | N | VAL | 5308 | 53.830 | 15.281 | 28.580 | 1.00 | 45.14 |
| ATOM | 10428 | CA | VAL | 5308 | 53.126 | 16.333 | 29.311 | 1.00 | 43.39 |
| ATOM | 10429 | CB | VAL | 5308 | 52.628 | 15.820 | 30.679 | 1.00 | 42.70 |
| ATOM | 10430 | CG1 | VAL | 5308 | 51.736 | 14.616 | 30.485 | 1.00 | 42.10 |
| ATOM | 10431 | CG2 | VAL | 5308 | 53.809 | 15.462 | 31.558 | 1.00 | 41.79 |
| ATOM | 10432 | C | VAL | 5308 | 54.029 | 17.537 | 29.554 | 1.00 | 42.39 |
| ATOM | 10433 | O | VAL | 5308 | 55.244 | 17.411 | 29.600 | 1.00 | 42.56 |
| ATOM | 10434 | N | GLN | 5309 | 53.423 | 18.704 | 29.709 | 1.00 | 41.50 |
| ATOM | 10435 | CA | GLN | 5309 | 54.166 | 19.928 | 29.972 | 1.00 | 39.83 |
| ATOM | 10436 | CB | GLN | 5309 | 53.954 | 20.927 | 28.831 | 1.00 | 39.25 |
| ATOM | 10437 | CG | GLN | 5309 | 54.891 | 22.122 | 28.877 | 1.00 | 38.16 |
| ATOM | 10438 | CD | GLN | 5309 | 54.402 | 23.307 | 28.059 | 1.00 | 37.21 |
| ATOM | 10439 | OE1 | GLN | 5309 | 53.810 | 23.144 | 27.002 | 1.00 | 37.23 |
| ATOM | 10440 | NE2 | GLN | 5309 | 54.666 | 24.506 | 28.548 | 1.00 | 37.17 |
| ATOM | 10441 | C | GLN | 5309 | 53.607 | 20.510 | 31.272 | 1.00 | 39.26 |
| ATOM | 10442 | O | GLN | 5309 | 52.402 | 20.769 | 31.358 | 1.00 | 39.28 |
| ATOM | 10443 | N | ILE | 5310 | 54.462 | 20.693 | 32.280 | 1.00 | 38.19 |
| ATOM | 10444 | CA | ILE | 5310 | 54.019 | 21.254 | 33.553 | 1.00 | 36.78 |
| ATOM | 10445 | CB | ILE | 5310 | 55.074 | 21.128 | 34.674 | 1.00 | 35.58 |
| ATOM | 10446 | CG2 | ILE | 5310 | 54.447 | 21.505 | 36.009 | 1.00 | 35.16 |
| ATOM | 10447 | CG1 | ILE | 5310 | 55.633 | 19.704 | 34.747 | 1.00 | 35.17 |
| ATOM | 10448 | CD1 | ILE | 5310 | 54.622 | 18.636 | 34.858 | 1.00 | 34.52 |
| ATOM | 10449 | C | ILE | 5310 | 53.767 | 22.733 | 33.326 | 1.00 | 37.12 |
| ATOM | 10450 | O | ILE | 5310 | 54.682 | 23.480 | 32.990 | 1.00 | 37.92 |
| ATOM | 10451 | N | LEU | 5311 | 52.526 | 23.161 | 33.511 | 1.00 | 36.70 |
| ATOM | 10452 | CA | LEU | 5311 | 52.185 | 24.561 | 33.295 | 1.00 | 36.53 |
| ATOM | 10453 | CB | LEU | 5311 | 50.837 | 24.682 | 32.593 | 1.00 | 36.05 |
| ATOM | 10454 | CG | LEU | 5311 | 50.810 | 24.107 | 31.185 | 1.00 | 36.98 |
| ATOM | 10455 | CD1 | LEU | 5311 | 49.416 | 24.251 | 30.587 | 1.00 | 37.07 |
| ATOM | 10456 | CD2 | LEU | 5311 | 51.851 | 24.827 | 30.337 | 1.00 | 36.73 |
| ATOM | 10457 | C | LEU | 5311 | 52.122 | 25.358 | 34.568 | 1.00 | 36.28 |
| ATOM | 10458 | O | LEU | 5311 | 52.248 | 26.571 | 34.535 | 1.00 | 36.46 |
| ATOM | 10459 | N | LYS | 5312 | 51.925 | 24.680 | 35.692 | 1.00 | 36.20 |
| ATOM | 10460 | CA | LYS | 5312 | 51.804 | 25.362 | 36.969 | 1.00 | 35.17 |
| ATOM | 10461 | CB | LYS | 5312 | 50.410 | 25.972 | 37.047 | 1.00 | 34.80 |
| ATOM | 10462 | CG | LYS | 5312 | 50.255 | 27.144 | 37.980 | 1.00 | 34.34 |
| ATOM | 10463 | CD | LYS | 5312 | 48.890 | 27.753 | 37.774 | 1.00 | 33.27 |
| ATOM | 10464 | CE | LYS | 5312 | 48.691 | 28.993 | 38.612 | 1.00 | 33.15 |
| ATOM | 10465 | NZ | LYS | 5312 | 47.376 | 29.638 | 38.310 | 1.00 | 33.22 |
| ATOM | 10466 | C | LYS | 5312 | 52.013 | 24.342 | 38.086 | 1.00 | 35.15 |
| ATOM | 10467 | O | LYS | 5312 | 51.437 | 23.262 | 38.061 | 1.00 | 35.01 |
| ATOM | 10468 | N | THR | 5313 | 52.842 | 24.685 | 39.061 | 1.00 | 34.86 |
| ATOM | 10469 | CA | THR | 5313 | 53.126 | 23.776 | 40.154 | 1.00 | 35.35 |
| ATOM | 10470 | CB | THR | 5313 | 54.515 | 23.137 | 39.988 | 1.00 | 35.34 |
| ATOM | 10471 | OG1 | THR | 5313 | 54.557 | 22.419 | 38.750 | 1.00 | 36.77 |
| ATOM | 10472 | CG2 | THR | 5313 | 54.806 | 22.175 | 41.139 | 1.00 | 34.58 |
| ATOM | 10473 | C | THR | 5313 | 53.075 | 24.472 | 41.507 | 1.00 | 35.10 |
| ATOM | 10474 | O | THR | 5313 | 53.708 | 25.506 | 41.708 | 1.00 | 35.50 |
| ATOM | 10475 | N | ALA | 5314 | 52.322 | 23.893 | 42.436 | 1.00 | 34.64 |
| ATOM | 10476 | CA | ALA | 5314 | 52.198 | 24.457 | 43.776 | 1.00 | 34.20 |
| ATOM | 10477 | CB | ALA | 5314 | 51.149 | 23.696 | 44.580 | 1.00 | 33.71 |
| ATOM | 10478 | C | ALA | 5314 | 53.537 | 24.397 | 44.496 | 1.00 | 33.91 |
| ATOM | 10479 | O | ALA | 5314 | 54.336 | 23.490 | 44.279 | 1.00 | 33.61 |
| ATOM | 10480 | N | GLY | 5315 | 53.768 | 25.378 | 45.357 | 1.00 | 33.75 |
| ATOM | 10481 | CA | GLY | 5315 | 54.999 | 25.439 | 46.113 | 1.00 | 34.07 |
| ATOM | 10482 | C | GLY | 5315 | 55.047 | 26.779 | 46.803 | 1.00 | 34.87 |
| ATOM | 10483 | O | GLY | 5315 | 54.070 | 27.525 | 46.778 | 1.00 | 34.86 |
| ATOM | 10484 | N | VAL | 5316 | 56.175 | 27.115 | 47.409 | 1.00 | 35.44 |
| ATOM | 10485 | CA | VAL | 5316 | 56.252 | 28.394 | 48.089 | 1.00 | 36.36 |
| ATOM | 10486 | CB | VAL | 5316 | 57.536 | 28.547 | 48.883 | 1.00 | 36.51 |
| ATOM | 10487 | CG1 | VAL | 5316 | 57.359 | 29.678 | 49.872 | 1.00 | 38.83 |
| ATOM | 10488 | CG2 | VAL | 5316 | 57.856 | 27.280 | 49.615 | 1.00 | 38.27 |
| ATOM | 10489 | C | VAL | 5316 | 56.145 | 29.586 | 47.153 | 1.00 | 36.55 |
| ATOM | 10490 | O | VAL | 5316 | 55.750 | 30.667 | 47.571 | 1.00 | 36.85 |
| ATOM | 10491 | N | ASN | 5317 | 56.488 | 29.391 | 45.886 | 1.00 | 37.03 |
| ATOM | 10492 | CA | ASN | 5317 | 56.437 | 30.473 | 44.917 | 1.00 | 37.57 |
| ATOM | 10493 | CB | ASN | 5317 | 57.566 | 30.290 | 43.899 | 1.00 | 38.06 |
| ATOM | 10494 | CG | ASN | 5317 | 58.940 | 30.299 | 44.554 | 1.00 | 39.05 |
| ATOM | 10495 | OD1 | ASN | 5317 | 59.710 | 29.345 | 44.417 | 1.00 | 40.19 |
| ATOM | 10496 | ND2 | ASN | 5317 | 59.253 | 31.376 | 45.272 | 1.00 | 38.32 |
| ATOM | 10497 | C | ASN | 5317 | 55.084 | 30.586 | 44.219 | 1.00 | 37.44 |
| ATOM | 10498 | O | ASN | 5317 | 54.761 | 31.610 | 43.624 | 1.00 | 37.63 |
| ATOM | 10499 | N | THR | 5318 | 54.295 | 29.527 | 44.288 | 1.00 | 37.35 |
| ATOM | 10500 | CA | THR | 5318 | 52.973 | 29.538 | 43.685 | 1.00 | 37.60 |
| ATOM | 10501 | CB | THR | 5318 | 52.935 | 28.712 | 42.410 | 1.00 | 37.44 |
| ATOM | 10502 | OG1 | THR | 5318 | 54.147 | 28.933 | 41.670 | 1.00 | 39.08 |
| ATOM | 10503 | CG2 | THR | 5318 | 51.743 | 29.125 | 41.565 | 1.00 | 36.48 |
| ATOM | 10504 | C | THR | 5318 | 52.059 | 28.915 | 44.717 | 1.00 | 37.45 |
| ATOM | 10505 | O | THR | 5318 | 51.798 | 27.713 | 44.684 | 1.00 | 38.01 |
| ATOM | 10506 | N | THR | 5319 | 51.583 | 29.735 | 45.646 | 1.00 | 37.04 |
| ATOM | 10507 | CA | THR | 5319 | 50.740 | 29.213 | 46.701 | 1.00 | 37.61 |
| ATOM | 10508 | CB | THR | 5319 | 50.607 | 30.203 | 47.879 | 1.00 | 37.70 |
| ATOM | 10509 | OG1 | THR | 5319 | 49.967 | 31.411 | 47.451 | 1.00 | 37.73 |
| ATOM | 10510 | CG2 | THR | 5319 | 51.996 | 30.521 | 48.435 | 1.00 | 37.21 |
| ATOM | 10511 | C | THR | 5319 | 49.380 | 28.724 | 46.247 | 1.00 | 38.11 |
| ATOM | 10512 | O | THR | 5319 | 48.949 | 28.964 | 45.109 | 1.00 | 38.17 |
| ATOM | 10513 | N | ASP | 5320 | 48.729 | 27.996 | 47.145 | 1.00 | 38.19 |
| ATOM | 10514 | CA | ASP | 5320 | 47.426 | 27.411 | 46.884 | 1.00 | 39.44 |
| ATOM | 10515 | CB | ASP | 5320 | 46.977 | 26.617 | 48.107 | 1.00 | 38.79 |
| ATOM | 10516 | CG | ASP | 5320 | 47.929 | 25.480 | 48.438 | 1.00 | 39.99 |
| ATOM | 10517 | OD1 | ASP | 5320 | 48.404 | 24.820 | 47.487 | 1.00 | 39.90 |
| ATOM | 10518 | OD2 | ASP | 5320 | 48.202 | 25.235 | 49.637 | 1.00 | 40.11 |
| ATOM | 10519 | C | ASP | 5320 | 46.354 | 28.416 | 46.497 | 1.00 | 40.12 |
| ATOM | 10520 | O | ASP | 5320 | 45.424 | 28.078 | 45.765 | 1.00 | 40.26 |
| ATOM | 10521 | N | LYS | 5321 | 46.486 | 29.650 | 46.983 | 1.00 | 40.77 |
| ATOM | 10522 | CA | LYS | 5321 | 45.507 | 30.698 | 46.686 | 1.00 | 41.52 |
| ATOM | 10523 | CB | LYS | 5321 | 46.017 | 32.115 | 47.038 | 1.00 | 41.56 |
| ATOM | 10524 | CG | LYS | 5321 | 46.784 | 32.335 | 48.334 | 1.00 | 42.74 |
| ATOM | 10525 | CD | LYS | 5321 | 47.179 | 33.831 | 48.437 | 1.00 | 43.43 |
| ATOM | 10526 | CE | LYS | 5321 | 48.194 | 34.141 | 49.554 | 1.00 | 44.17 |
| ATOM | 10527 | NZ | LYS | 5321 | 49.655 | 34.060 | 49.149 | 1.00 | 45.09 |
| ATOM | 10528 | C | LYS | 5321 | 45.187 | 30.735 | 45.196 | 1.00 | 41.41 |
| ATOM | 10529 | O | LYS | 5321 | 44.070 | 31.066 | 44.801 | 1.00 | 41.23 |
| ATOM | 10530 | N | GLU | 5322 | 46.174 | 30.400 | 44.375 | 1.00 | 41.24 |
| ATOM | 10531 | CA | GLU | 5322 | 45.999 | 30.472 | 42.937 | 1.00 | 41.66 |
| ATOM | 10532 | CB | GLU | 5322 | 46.980 | 31.513 | 42.374 | 1.00 | 42.60 |
| ATOM | 10533 | CG | GLU | 5322 | 48.408 | 31.390 | 42.902 | 1.00 | 43.38 |
| ATOM | 10534 | CD | GLU | 5322 | 49.211 | 32.691 | 42.753 | 1.00 | 45.10 |
| ATOM | 10535 | OE1 | GLU | 5322 | 49.546 | 33.078 | 41.604 | 1.00 | 45.81 |
| ATOM | 10536 | OE2 | GLU | 5322 | 49.504 | 33.332 | 43.792 | 1.00 | 44.28 |
| ATOM | 10537 | C | GLU | 5322 | 46.132 | 29.186 | 42.148 | 1.00 | 40.49 |
| ATOM | 10538 | O | GLU | 5322 | 46.142 | 29.222 | 40.921 | 1.00 | 40.81 |
| ATOM | 10539 | N | MSE | 5323 | 46.211 | 28.053 | 42.830 | 1.00 | 39.13 |
| ATOM | 10540 | CA | MSE | 5323 | 46.382 | 26.801 | 42.119 | 1.00 | 37.91 |
| ATOM | 10541 | CB | MSE | 5323 | 47.179 | 25.839 | 42.993 | 1.00 | 35.02 |
| ATOM | 10542 | CG | MSE | 5323 | 48.634 | 26.283 | 43.231 | 1.00 | 30.86 |
| ATOM | 10543 | SE | MSE | 5323 | 49.630 | 26.327 | 41.722 | 1.00 | 24.89 |
| ATOM | 10544 | CE | MSE | 5323 | 49.361 | 24.667 | 41.069 | 1.00 | 24.32 |
| ATOM | 10545 | C | MSE | 5323 | 45.098 | 26.148 | 41.615 | 1.00 | 38.45 |
| ATOM | 10546 | O | MSE | 5323 | 45.150 | 25.223 | 40.813 | 1.00 | 38.73 |
| ATOM | 10547 | N | GLU | 5324 | 43.945 | 26.634 | 42.059 | 1.00 | 38.47 |
| ATOM | 10548 | CA | GLU | 5324 | 42.691 | 26.051 | 41.615 | 1.00 | 39.01 |
| ATOM | 10549 | CB | GLU | 5324 | 41.633 | 26.186 | 42.716 | 1.00 | 38.16 |
| ATOM | 10550 | CG | GLU | 5324 | 42.025 | 25.460 | 44.005 | 1.00 | 38.69 |
| ATOM | 10551 | CD | GLU | 5324 | 40.867 | 25.304 | 45.000 | 1.00 | 39.49 |
| ATOM | 10552 | OE1 | GLU | 5324 | 40.313 | 26.350 | 45.424 | 1.00 | 39.47 |
| ATOM | 10553 | OE2 | GLU | 5324 | 40.521 | 24.139 | 45.354 | 1.00 | 37.80 |
| ATOM | 10554 | C | GLU | 5324 | 42.195 | 26.641 | 40.285 | 1.00 | 39.36 |
| ATOM | 10555 | O | GLU | 5324 | 41.082 | 26.359 | 39.841 | 1.00 | 39.99 |
| ATOM | 10556 | N | VAL | 5325 | 43.031 | 27.436 | 39.630 | 1.00 | 39.05 |
| ATOM | 10557 | CA | VAL | 5325 | 42.634 | 28.022 | 38.363 | 1.00 | 39.21 |
| ATOM | 10558 | CB | VAL | 5325 | 41.984 | 29.413 | 38.607 | 1.00 | 38.69 |
| ATOM | 10559 | CG1 | VAL | 5325 | 42.989 | 30.363 | 39.194 | 1.00 | 38.98 |
| ATOM | 10560 | CG2 | VAL | 5325 | 41.422 | 29.968 | 37.322 | 1.00 | 39.54 |
| ATOM | 10561 | C | VAL | 5325 | 43.829 | 28.137 | 37.415 | 1.00 | 39.16 |
| ATOM | 10562 | O | VAL | 5325 | 44.930 | 28.449 | 37.835 | 1.00 | 39.92 |
| ATOM | 10563 | N | LEU | 5326 | 43.612 | 27.849 | 36.139 | 1.00 | 39.09 |
| ATOM | 10564 | CA | LEU | 5326 | 44.667 | 27.949 | 35.144 | 1.00 | 38.77 |
| ATOM | 10565 | CB | LEU | 5326 | 44.929 | 26.593 | 34.490 | 1.00 | 37.52 |
| ATOM | 10566 | CG | LEU | 5326 | 45.967 | 26.607 | 33.367 | 1.00 | 35.59 |
| ATOM | 10567 | CD1 | LEU | 5326 | 47.339 | 26.893 | 33.944 | 1.00 | 34.49 |
| ATOM | 10568 | CD2 | LEU | 5326 | 45.959 | 25.282 | 32.650 | 1.00 | 34.98 |
| ATOM | 10569 | C | LEU | 5326 | 44.225 | 28.940 | 34.082 | 1.00 | 39.03 |
| ATOM | 10570 | O | LEU | 5326 | 43.193 | 28.752 | 33.448 | 1.00 | 38.82 |
| ATOM | 10571 | N | HIS | 5327 | 45.011 | 29.990 | 33.889 | 1.00 | 39.93 |
| ATOM | 10572 | CA | HIS | 5327 | 44.689 | 31.012 | 32.903 | 1.00 | 40.97 |
| ATOM | 10573 | CB | HIS | 5327 | 44.879 | 32.393 | 33.508 | 1.00 | 39.76 |
| ATOM | 10574 | CG | HIS | 5327 | 44.016 | 32.640 | 34.697 | 1.00 | 39.04 |
| ATOM | 10575 | CD2 | HIS | 5327 | 44.305 | 32.658 | 36.019 | 1.00 | 39.11 |
| ATOM | 10576 | ND1 | HIS | 5327 | 42.659 | 32.851 | 34.596 | 1.00 | 38.85 |
| ATOM | 10577 | CE1 | HIS | 5327 | 42.147 | 32.992 | 35.806 | 1.00 | 39.06 |
| ATOM | 10578 | NE2 | HIS | 5327 | 43.125 | 32.878 | 36.687 | 1.00 | 39.71 |
| ATOM | 10579 | C | HIS | 5327 | 45.508 | 30.902 | 31.630 | 1.00 | 42.10 |
| ATOM | 10580 | O | HIS | 5327 | 46.727 | 30.749 | 31.661 | 1.00 | 42.54 |
| ATOM | 10581 | N | LEU | 5328 | 44.814 | 30.963 | 30.503 | 1.00 | 43.11 |
| ATOM | 10582 | CA | LEU | 5328 | 45.447 | 30.905 | 29.194 | 1.00 | 43.92 |
| ATOM | 10583 | CB | LEU | 5328 | 44.996 | 29.659 | 28.416 | 1.00 | 42.36 |
| ATOM | 10584 | CG | LEU | 5328 | 45.430 | 28.265 | 28.901 | 1.00 | 41.94 |
| ATOM | 10585 | CD1 | LEU | 5328 | 44.775 | 27.218 | 28.013 | 1.00 | 41.34 |
| ATOM | 10586 | CD2 | LEU | 5328 | 46.945 | 28.107 | 28.861 | 1.00 | 39.71 |
| ATOM | 10587 | C | LEU | 5328 | 45.012 | 32.180 | 28.463 | 1.00 | 45.53 |
| ATOM | 10588 | O | LEU | 5328 | 43.813 | 32.444 | 28.296 | 1.00 | 45.69 |
| ATOM | 10589 | N | ARG | 5329 | 45.990 | 32.973 | 28.038 | 1.00 | 46.93 |
| ATOM | 10590 | CA | ARG | 5329 | 45.715 | 34.235 | 27.363 | 1.00 | 48.65 |
| ATOM | 10591 | CB | ARG | 5329 | 46.580 | 35.342 | 27.978 | 1.00 | 50.02 |
| ATOM | 10592 | CG | ARG | 5329 | 46.280 | 36.747 | 27.467 | 1.00 | 52.96 |
| ATOM | 10593 | CD | ARG | 5329 | 47.288 | 37.767 | 27.975 | 1.00 | 54.85 |
| ATOM | 10594 | NE | ARG | 5329 | 46.940 | 39.133 | 27.577 | 1.00 | 57.63 |
| ATOM | 10595 | CZ | ARG | 5329 | 47.744 | 40.188 | 27.704 | 1.00 | 59.28 |
| ATOM | 10596 | NH1 | ARG | 5329 | 48.956 | 40.042 | 28.216 | 1.00 | 59.15 |
| ATOM | 10597 | NH2 | ARG | 5329 | 47.335 | 41.396 | 27.312 | 1.00 | 59.24 |
| ATOM | 10598 | C | ARG | 5329 | 45.966 | 34.165 | 25.864 | 1.00 | 48.65 |
| ATOM | 10599 | O | ARG | 5329 | 46.919 | 33.534 | 25.417 | 1.00 | 48.60 |
| ATOM | 10600 | N | ASN | 5330 | 45.106 | 34.836 | 25.100 | 1.00 | 49.38 |
| ATOM | 10601 | CA | ASN | 5330 | 45.207 | 34.875 | 23.644 | 1.00 | 49.52 |
| ATOM | 10602 | CB | ASN | 5330 | 46.185 | 35.979 | 23.232 | 1.00 | 50.59 |
| ATOM | 10603 | CG | ASN | 5330 | 46.386 | 36.055 | 21.736 | 1.00 | 51.66 |
| ATOM | 10604 | OD1 | ASN | 5330 | 47.326 | 35.465 | 21.195 | 1.00 | 52.16 |
| ATOM | 10605 | ND2 | ASN | 5330 | 45.497 | 36.773 | 21.052 | 1.00 | 51.60 |
| ATOM | 10606 | C | ASN | 5330 | 45.643 | 33.512 | 23.116 | 1.00 | 49.46 |
| ATOM | 10607 | O | ASN | 5330 | 46.764 | 33.328 | 22.645 | 1.00 | 48.80 |
| ATOM | 10608 | N | VAL | 5331 | 44.728 | 32.554 | 23.199 | 1.00 | 49.96 |
| ATOM | 10609 | CA | VAL | 5331 | 45.007 | 31.187 | 22.776 | 1.00 | 49.78 |
| ATOM | 10610 | CB | VAL | 5331 | 43.884 | 30.224 | 23.232 | 1.00 | 49.78 |
| ATOM | 10611 | CG1 | VAL | 5331 | 43.733 | 30.281 | 24.741 | 1.00 | 49.25 |
| ATOM | 10612 | CG2 | VAL | 5331 | 42.576 | 30.594 | 22.555 | 1.00 | 49.72 |
| ATOM | 10613 | C | VAL | 5331 | 45.205 | 31.012 | 21.282 | 1.00 | 49.34 |
| ATOM | 10614 | O | VAL | 5331 | 44.521 | 31.630 | 20.473 | 1.00 | 48.98 |
| ATOM | 10615 | N | SER | 5332 | 46.157 | 30.156 | 20.932 | 1.00 | 49.63 |
| ATOM | 10616 | CA | SER | 5332 | 46.454 | 29.853 | 19.534 | 1.00 | 50.14 |
| ATOM | 10617 | CB | SER | 5332 | 47.964 | 29.865 | 19.276 | 1.00 | 49.77 |
| ATOM | 10618 | OG | SER | 5332 | 48.565 | 28.655 | 19.706 | 1.00 | 48.75 |
| ATOM | 10619 | C | SER | 5332 | 45.923 | 28.453 | 19.260 | 1.00 | 50.29 |
| ATOM | 10620 | O | SER | 5332 | 45.307 | 27.843 | 20.130 | 1.00 | 50.42 |
| ATOM | 10621 | N | PHE | 5333 | 46.156 | 27.943 | 18.055 | 1.00 | 50.47 |
| ATOM | 10622 | CA | PHE | 5333 | 45.700 | 26.596 | 17.717 | 1.00 | 50.89 |
| ATOM | 10623 | CB | PHE | 5333 | 45.786 | 26.332 | 16.210 | 1.00 | 50.04 |
| ATOM | 10624 | CG | PHE | 5333 | 44.717 | 27.012 | 15.405 | 1.00 | 50.14 |
| ATOM | 10625 | CD1 | PHE | 5333 | 45.049 | 27.992 | 14.467 | 1.00 | 50.12 |
| ATOM | 10626 | CD2 | PHE | 5333 | 43.379 | 26.673 | 15.576 | 1.00 | 49.78 |
| ATOM | 10627 | CE1 | PHE | 5333 | 44.064 | 28.621 | 13.712 | 1.00 | 49.70 |
| ATOM | 10628 | CE2 | PHE | 5333 | 42.386 | 27.296 | 14.826 | 1.00 | 50.11 |
| ATOM | 10629 | CZ | PHE | 5333 | 42.729 | 28.273 | 13.892 | 1.00 | 49.91 |
| ATOM | 10630 | C | PHE | 5333 | 46.579 | 25.588 | 18.433 | 1.00 | 50.99 |
| ATOM | 10631 | O | PHE | 5333 | 46.194 | 24.443 | 18.637 | 1.00 | 50.49 |
| ATOM | 10632 | N | GLU | 5334 | 47.770 | 26.029 | 18.806 | 1.00 | 51.82 |
| ATOM | 10633 | CA | GLU | 5334 | 48.715 | 25.175 | 19.496 | 1.00 | 52.41 |
| ATOM | 10634 | CB | GLU | 5334 | 50.091 | 25.838 | 19.472 | 1.00 | 54.73 |
| ATOM | 10635 | CG | GLU | 5334 | 50.456 | 26.297 | 18.059 | 1.00 | 58.68 |
| ATOM | 10636 | CD | GLU | 5334 | 51.555 | 27.355 | 18.015 | 1.00 | 60.96 |
| ATOM | 10637 | OE1 | GLU | 5334 | 51.713 | 27.982 | 16.937 | 1.00 | 61.63 |
| ATOM | 10638 | OE2 | GLU | 5334 | 52.255 | 27.554 | 19.041 | 1.00 | 61.76 |
| ATOM | 10639 | C | GLU | 5334 | 48.236 | 24.921 | 20.921 | 1.00 | 51.27 |
| ATOM | 10640 | O | GLU | 5334 | 48.478 | 23.852 | 21.475 | 1.00 | 51.73 |
| ATOM | 10641 | N | ASP | 5335 | 47.530 | 25.889 | 21.499 | 1.00 | 49.21 |
| ATOM | 10642 | CA | ASP | 5335 | 47.023 | 25.745 | 22.857 | 1.00 | 47.05 |
| ATOM | 10643 | CB | ASP | 5335 | 46.569 | 27.106 | 23.390 | 1.00 | 48.06 |
| ATOM | 10644 | CG | ASP | 5335 | 47.700 | 28.125 | 23.411 | 1.00 | 49.15 |
| ATOM | 10645 | OD1 | ASP | 5335 | 48.787 | 27.801 | 23.949 | 1.00 | 49.02 |
| ATOM | 10646 | OD2 | ASP | 5335 | 47.505 | 29.251 | 22.894 | 1.00 | 50.01 |
| ATOM | 10647 | C | ASP | 5335 | 45.898 | 24.714 | 22.983 | 1.00 | 44.98 |
| ATOM | 10648 | O | ASP | 5335 | 45.627 | 24.221 | 24.075 | 1.00 | 43.93 |
| ATOM | 10649 | N | ALA | 5336 | 45.251 | 24.381 | 21.871 | 1.00 | 42.80 |
| ATOM | 10650 | CA | ALA | 5336 | 44.182 | 23.391 | 21.896 | 1.00 | 41.20 |
| ATOM | 10651 | CB | ALA | 5336 | 43.589 | 23.233 | 20.516 | 1.00 | 41.10 |
| ATOM | 10652 | C | ALA | 5336 | 44.756 | 22.062 | 22.376 | 1.00 | 40.12 |
| ATOM | 10653 | O | ALA | 5336 | 45.948 | 21.800 | 22.212 | 1.00 | 40.22 |
| ATOM | 10654 | N | GLY | 5337 | 43.916 | 21.228 | 22.978 | 1.00 | 38.43 |
| ATOM | 10655 | CA | GLY | 5337 | 44.389 | 19.949 | 23.472 | 1.00 | 37.45 |
| ATOM | 10656 | C | GLY | 5337 | 43.853 | 19.574 | 24.844 | 1.00 | 36.89 |
| ATOM | 10657 | O | GLY | 5337 | 42.906 | 20.176 | 25.339 | 1.00 | 37.17 |
| ATOM | 10658 | N | GLU | 5338 | 44.471 | 18.578 | 25.466 | 1.00 | 35.83 |
| ATOM | 10659 | CA | GLU | 5338 | 44.041 | 18.106 | 26.771 | 1.00 | 34.51 |
| ATOM | 10660 | CB | GLU | 5338 | 44.128 | 16.580 | 26.811 | 1.00 | 35.17 |
| ATOM | 10661 | CG | GLU | 5338 | 43.802 | 15.964 | 28.156 | 1.00 | 36.27 |
| ATOM | 10662 | CD | GLU | 5338 | 43.407 | 14.510 | 28.032 | 1.00 | 37.64 |
| ATOM | 10663 | OE1 | GLU | 5338 | 44.073 | 13.783 | 27.268 | 1.00 | 38.67 |
| ATOM | 10664 | OE2 | GLU | 5338 | 42.434 | 14.085 | 28.697 | 1.00 | 38.34 |
| ATOM | 10665 | C | GLU | 5338 | 44.826 | 18.695 | 27.936 | 1.00 | 33.55 |
| ATOM | 10666 | O | GLU | 5338 | 46.054 | 18.663 | 27.950 | 1.00 | 33.36 |
| ATOM | 10667 | N | TYR | 5339 | 44.095 | 19.225 | 28.915 | 1.00 | 32.07 |
| ATOM | 10668 | CA | TYR | 5339 | 44.679 | 19.822 | 30.111 | 1.00 | 30.50 |
| ATOM | 10669 | CB | TYR | 5339 | 44.175 | 21.248 | 30.296 | 1.00 | 29.26 |
| ATOM | 10670 | CG | TYR | 5339 | 44.729 | 22.197 | 29.270 | 1.00 | 28.72 |
| ATOM | 10671 | CD1 | TYR | 5339 | 44.264 | 22.186 | 27.959 | 1.00 | 27.56 |
| ATOM | 10672 | CE1 | TYR | 5339 | 44.817 | 23.025 | 27.000 | 1.00 | 28.07 |
| ATOM | 10673 | CD2 | TYR | 5339 | 45.765 | 23.076 | 29.597 | 1.00 | 28.30 |
| ATOM | 10674 | CE2 | TYR | 5339 | 46.326 | 23.911 | 28.647 | 1.00 | 27.11 |
| ATOM | 10675 | CZ | TYR | 5339 | 45.853 | 23.886 | 27.354 | 1.00 | 27.63 |
| ATOM | 10676 | OH | TYR | 5339 | 46.409 | 24.726 | 26.417 | 1.00 | 28.44 |
| ATOM | 10677 | C | TYR | 5339 | 44.294 | 18.998 | 31.317 | 1.00 | 30.19 |
| ATOM | 10678 | O | TYR | 5339 | 43.151 | 18.570 | 31.442 | 1.00 | 30.80 |
| ATOM | 10679 | N | THR | 5340 | 45.243 | 18.792 | 32.219 | 1.00 | 29.67 |
| ATOM | 10680 | CA | THR | 5340 | 44.981 | 17.980 | 33.389 | 1.00 | 29.38 |
| ATOM | 10681 | CB | THR | 5340 | 45.785 | 16.661 | 33.319 | 1.00 | 28.59 |
| ATOM | 10682 | OG1 | THR | 5340 | 45.341 | 15.892 | 32.198 | 1.00 | 27.25 |
| ATOM | 10683 | CG2 | THR | 5340 | 45.603 | 15.851 | 34.585 | 1.00 | 27.89 |
| ATOM | 10684 | C | THR | 5340 | 45.312 | 18.657 | 34.698 | 1.00 | 29.54 |
| ATOM | 10685 | O | THR | 5340 | 46.308 | 19.360 | 34.807 | 1.00 | 29.41 |
| ATOM | 10686 | N | CYS | 5341 | 44.456 | 18.438 | 35.690 | 1.00 | 30.31 |
| ATOM | 10687 | CA | CYS | 5341 | 44.678 | 18.965 | 37.031 | 1.00 | 31.02 |
| ATOM | 10688 | CB | CYS | 5341 | 43.423 | 19.612 | 37.608 | 1.00 | 31.34 |
| ATOM | 10689 | SG | CYS | 5341 | 43.643 | 20.158 | 39.319 | 1.00 | 35.38 |
| ATOM | 10690 | C | CYS | 5341 | 45.040 | 17.751 | 37.868 | 1.00 | 31.05 |
| ATOM | 10691 | O | CYS | 5341 | 44.210 | 16.880 | 38.111 | 1.00 | 30.92 |
| ATOM | 10692 | N | LEU | 5342 | 46.291 | 17.699 | 38.297 | 1.00 | 31.18 |
| ATOM | 10693 | CA | LEU | 5342 | 46.786 | 16.584 | 39.085 | 1.00 | 31.35 |
| ATOM | 10694 | CB | LEU | 5342 | 48.126 | 16.138 | 38.497 | 1.00 | 32.96 |
| ATOM | 10695 | CG | LEU | 5342 | 49.015 | 15.131 | 39.227 | 1.00 | 34.97 |
| ATOM | 10696 | CD1 | LEU | 5342 | 48.292 | 13.806 | 39.404 | 1.00 | 35.93 |
| ATOM | 10697 | CD2 | LEU | 5342 | 50.298 | 14.946 | 38.424 | 1.00 | 35.58 |
| ATOM | 10698 | C | LEU | 5342 | 46.933 | 16.978 | 40.548 | 1.00 | 30.60 |
| ATOM | 10699 | O | LEU | 5342 | 47.499 | 18.013 | 40.860 | 1.00 | 31.31 |
| ATOM | 10700 | N | ALA | 5343 | 46.415 | 16.161 | 41.453 | 1.00 | 29.71 |
| ATOM | 10701 | CA | ALA | 5343 | 46.518 | 16.472 | 42.878 | 1.00 | 29.07 |
| ATOM | 10702 | CB | ALA | 5343 | 45.202 | 17.045 | 43.396 | 1.00 | 28.09 |
| ATOM | 10703 | C | ALA | 5343 | 46.890 | 15.232 | 43.675 | 1.00 | 28.65 |
| ATOM | 10704 | O | ALA | 5343 | 46.284 | 14.174 | 43.512 | 1.00 | 29.57 |
| ATOM | 10705 | N | GLY | 5344 | 47.887 | 15.353 | 44.539 | 1.00 | 27.49 |
| ATOM | 10706 | CA | GLY | 5344 | 48.271 | 14.204 | 45.326 | 1.00 | 26.66 |
| ATOM | 10707 | C | GLY | 5344 | 48.711 | 14.525 | 46.736 | 1.00 | 25.98 |
| ATOM | 10708 | O | GLY | 5344 | 49.041 | 15.663 | 47.060 | 1.00 | 25.70 |
| ATOM | 10709 | N | ASN | 5345 | 48.687 | 13.507 | 47.585 | 1.00 | 26.21 |
| ATOM | 10710 | CA | ASN | 5345 | 49.123 | 13.633 | 48.971 | 1.00 | 26.47 |
| ATOM | 10711 | CB | ASN | 5345 | 47.942 | 13.887 | 49.922 | 1.00 | 25.09 |
| ATOM | 10712 | CG | ASN | 5345 | 46.852 | 12.827 | 49.825 | 1.00 | 23.69 |
| ATOM | 10713 | OD1 | ASN | 5345 | 47.089 | 11.705 | 49.378 | 1.00 | 23.06 |
| ATOM | 10714 | ND2 | ASN | 5345 | 45.653 | 13.181 | 50.265 | 1.00 | 22.82 |
| ATOM | 10715 | C | ASN | 5345 | 49.809 | 12.310 | 49.276 | 1.00 | 27.46 |
| ATOM | 10716 | O | ASN | 5345 | 49.868 | 11.437 | 48.401 | 1.00 | 27.69 |
| ATOM | 10717 | N | SER | 5346 | 50.325 | 12.139 | 50.489 | 1.00 | 28.05 |
| ATOM | 10718 | CA | SER | 5346 | 51.042 | 10.906 | 50.814 | 1.00 | 28.97 |
| ATOM | 10719 | CB | SER | 5346 | 51.475 | 10.875 | 52.285 | 1.00 | 29.75 |
| ATOM | 10720 | OG | SER | 5346 | 50.380 | 10.615 | 53.159 | 1.00 | 32.69 |
| ATOM | 10721 | C | SER | 5346 | 50.250 | 9.649 | 50.509 | 1.00 | 28.96 |
| ATOM | 10722 | O | SER | 5346 | 50.824 | 8.634 | 50.126 | 1.00 | 28.66 |
| ATOM | 10723 | N | ILE | 5347 | 48.931 | 9.721 | 50.657 | 1.00 | 29.00 |
| ATOM | 10724 | CA | ILE | 5347 | 48.071 | 8.569 | 50.419 | 1.00 | 27.89 |
| ATOM | 10725 | CB | ILE | 5347 | 46.738 | 8.739 | 51.123 | 1.00 | 27.07 |
| ATOM | 10726 | CG2 | ILE | 5347 | 45.911 | 7.464 | 50.983 | 1.00 | 25.90 |
| ATOM | 10727 | CG1 | ILE | 5347 | 46.976 | 9.091 | 52.589 | 1.00 | 24.45 |
| ATOM | 10728 | CD1 | ILE | 5347 | 45.735 | 9.607 | 53.270 | 1.00 | 23.36 |
| ATOM | 10729 | C | ILE | 5347 | 47.788 | 8.220 | 48.966 | 1.00 | 29.21 |
| ATOM | 10730 | O | ILE | 5347 | 47.604 | 7.042 | 48.651 | 1.00 | 30.36 |
| ATOM | 10731 | N | GLY | 5348 | 47.743 | 9.209 | 48.074 | 1.00 | 29.73 |
| ATOM | 10732 | CA | GLY | 5348 | 47.475 | 8.901 | 46.675 | 1.00 | 29.73 |
| ATOM | 10733 | C | GLY | 5348 | 47.340 | 10.075 | 45.717 | 1.00 | 30.60 |
| ATOM | 10734 | O | GLY | 5348 | 47.395 | 11.240 | 46.117 | 1.00 | 30.60 |
| ATOM | 10735 | N | LEU | 5349 | 47.139 | 9.750 | 44.441 | 1.00 | 30.92 |
| ATOM | 10736 | CA | LEU | 5349 | 47.013 | 10.726 | 43.363 | 1.00 | 30.55 |
| ATOM | 10737 | CB | LEU | 5349 | 48.031 | 10.386 | 42.282 | 1.00 | 31.76 |
| ATOM | 10738 | CG | LEU | 5349 | 48.948 | 11.465 | 41.695 | 1.00 | 33.88 |
| ATOM | 10739 | CD1 | LEU | 5349 | 49.933 | 11.961 | 42.746 | 1.00 | 33.59 |
| ATOM | 10740 | CD2 | LEU | 5349 | 49.709 | 10.869 | 40.507 | 1.00 | 34.91 |
| ATOM | 10741 | C | LEU | 5349 | 45.623 | 10.749 | 42.726 | 1.00 | 30.21 |
| ATOM | 10742 | O | LEU | 5349 | 44.940 | 9.732 | 42.673 | 1.00 | 30.37 |
| ATOM | 10743 | N | SER | 5350 | 45.205 | 11.915 | 42.244 | 1.00 | 29.66 |
| ATOM | 10744 | CA | SER | 5350 | 43.911 | 12.050 | 41.576 | 1.00 | 29.01 |
| ATOM | 10745 | CB | SER | 5350 | 42.830 | 12.532 | 42.533 | 1.00 | 29.18 |
| ATOM | 10746 | OG | SER | 5350 | 42.731 | 11.669 | 43.639 | 1.00 | 31.09 |
| ATOM | 10747 | C | SER | 5350 | 44.069 | 13.065 | 40.469 | 1.00 | 28.41 |
| ATOM | 10748 | O | SER | 5350 | 44.915 | 13.952 | 40.546 | 1.00 | 28.94 |
| ATOM | 10749 | N | HIS | 5351 | 43.259 | 12.941 | 39.431 | 1.00 | 27.34 |
| ATOM | 10750 | CA | HIS | 5351 | 43.347 | 13.880 | 38.339 | 1.00 | 27.39 |
| ATOM | 10751 | CB | HIS | 5351 | 44.525 | 13.526 | 37.427 | 1.00 | 27.17 |
| ATOM | 10752 | CG | HIS | 5351 | 44.294 | 12.310 | 36.584 | 1.00 | 27.78 |
| ATOM | 10753 | CD2 | HIS | 5351 | 43.762 | 12.174 | 35.345 | 1.00 | 28.03 |
| ATOM | 10754 | ND1 | HIS | 5351 | 44.591 | 11.033 | 37.015 | 1.00 | 28.73 |
| ATOM | 10755 | CE1 | HIS | 5351 | 44.252 | 10.166 | 36.077 | 1.00 | 28.75 |
| ATOM | 10756 | NE2 | HIS | 5351 | 43.745 | 10.833 | 35.054 | 1.00 | 28.11 |
| ATOM | 10757 | C | HIS | 5351 | 42.071 | 13.928 | 37.517 | 1.00 | 27.25 |
| ATOM | 10758 | O | HIS | 5351 | 41.343 | 12.951 | 37.427 | 1.00 | 27.38 |
| ATOM | 10759 | N | HIS | 5352 | 41.814 | 15.092 | 36.936 | 1.00 | 27.28 |
| ATOM | 10760 | CA | HIS | 5352 | 40.663 | 15.319 | 36.077 | 1.00 | 27.52 |
| ATOM | 10761 | CB | HIS | 5352 | 39.652 | 16.257 | 36.741 | 1.00 | 27.58 |
| ATOM | 10762 | CG | HIS | 5352 | 38.710 | 15.578 | 37.682 | 1.00 | 29.15 |
| ATOM | 10763 | CD2 | HIS | 5352 | 38.574 | 14.274 | 38.031 | 1.00 | 29.88 |
| ATOM | 10764 | ND1 | HIS | 5352 | 37.729 | 16.262 | 38.371 | 1.00 | 29.34 |
| ATOM | 10765 | CE1 | HIS | 5352 | 37.026 | 15.412 | 39.100 | 1.00 | 29.44 |
| ATOM | 10766 | NE2 | HIS | 5352 | 37.519 | 14.198 | 38.912 | 1.00 | 30.36 |
| ATOM | 10767 | C | HIS | 5352 | 41.236 | 15.982 | 34.834 | 1.00 | 27.51 |
| ATOM | 10768 | O | HIS | 5352 | 42.208 | 16.729 | 34.918 | 1.00 | 27.49 |
| ATOM | 10769 | N | SER | 5353 | 40.643 | 15.697 | 33.683 | 1.00 | 27.68 |
| ATOM | 10770 | CA | SER | 5353 | 41.111 | 16.267 | 32.434 | 1.00 | 27.54 |
| ATOM | 10771 | CB | SER | 5353 | 41.717 | 15.185 | 31.553 | 1.00 | 27.08 |
| ATOM | 10772 | OG | SER | 5353 | 42.889 | 14.667 | 32.143 | 1.00 | 28.50 |
| ATOM | 10773 | C | SER | 5353 | 39.985 | 16.936 | 31.690 | 1.00 | 28.16 |
| ATOM | 10774 | O | SER | 5353 | 38.817 | 16.595 | 31.863 | 1.00 | 27.80 |
| ATOM | 10775 | N | ALA | 5354 | 40.347 | 17.903 | 30.859 | 1.00 | 28.65 |
| ATOM | 10776 | CA | ALA | 5354 | 39.367 | 18.616 | 30.065 | 1.00 | 29.26 |
| ATOM | 10777 | CB | ALA | 5354 | 38.996 | 19.923 | 30.746 | 1.00 | 27.32 |
| ATOM | 10778 | C | ALA | 5354 | 40.007 | 18.880 | 28.716 | 1.00 | 29.71 |
| ATOM | 10779 | O | ALA | 5354 | 41.228 | 18.920 | 28.602 | 1.00 | 28.83 |
| ATOM | 10780 | N | TRP | 5355 | 39.177 | 19.050 | 27.696 | 1.00 | 31.34 |
| ATOM | 10781 | CA | TRP | 5355 | 39.677 | 19.320 | 26.370 | 1.00 | 32.31 |
| ATOM | 10782 | CB | TRP | 5355 | 39.032 | 18.371 | 25.364 | 1.00 | 33.27 |
| ATOM | 10783 | CG | TRP | 5355 | 39.900 | 18.191 | 24.176 | 1.00 | 34.94 |
| ATOM | 10784 | CD2 | TRP | 5355 | 40.813 | 17.113 | 23.955 | 1.00 | 35.22 |
| ATOM | 10785 | CE2 | TRP | 5355 | 41.562 | 17.426 | 22.797 | 1.00 | 35.65 |
| ATOM | 10786 | CE3 | TRP | 5355 | 41.077 | 15.913 | 24.631 | 1.00 | 35.07 |
| ATOM | 10787 | CD1 | TRP | 5355 | 40.116 | 19.094 | 23.156 | 1.00 | 34.85 |
| ATOM | 10788 | NE1 | TRP | 5355 | 41.117 | 18.639 | 22.328 | 1.00 | 35.38 |
| ATOM | 10789 | CZ2 | TRP | 5355 | 42.558 | 16.580 | 22.302 | 1.00 | 35.11 |
| ATOM | 10790 | CZ3 | TRP | 5355 | 42.063 | 15.078 | 24.140 | 1.00 | 33.64 |
| ATOM | 10791 | CH2 | TRP | 5355 | 42.792 | 15.415 | 22.988 | 1.00 | 34.18 |
| ATOM | 10792 | C | TRP | 5355 | 39.393 | 20.763 | 25.974 | 1.00 | 32.87 |
| ATOM | 10793 | O | TRP | 5355 | 38.312 | 21.284 | 26.231 | 1.00 | 32.22 |
| ATOM | 10794 | N | LEU | 5356 | 40.377 | 21.404 | 25.355 | 1.00 | 33.18 |
| ATOM | 10795 | CA | LEU | 5356 | 40.222 | 22.776 | 24.905 | 1.00 | 34.03 |
| ATOM | 10796 | CB | LEU | 5356 | 41.416 | 23.628 | 25.337 | 1.00 | 32.75 |
| ATOM | 10797 | CG | LEU | 5356 | 41.315 | 25.166 | 25.307 | 1.00 | 33.19 |
| ATOM | 10798 | CD1 | LEU | 5356 | 42.486 | 25.726 | 24.521 | 1.00 | 31.83 |
| ATOM | 10799 | CD2 | LEU | 5356 | 39.992 | 25.637 | 24.731 | 1.00 | 31.28 |
| ATOM | 10800 | C | LEU | 5356 | 40.140 | 22.764 | 23.383 | 1.00 | 34.84 |
| ATOM | 10801 | O | LEU | 5356 | 41.059 | 22.304 | 22.701 | 1.00 | 34.42 |
| ATOM | 10802 | N | THR | 5357 | 39.028 | 23.257 | 22.855 | 1.00 | 35.35 |
| ATOM | 10803 | CA | THR | 5357 | 38.827 | 23.321 | 21.415 | 1.00 | 36.20 |
| ATOM | 10804 | CB | THR | 5357 | 37.442 | 22.776 | 21.028 | 1.00 | 35.85 |
| ATOM | 10805 | OG1 | THR | 5357 | 37.380 | 21.377 | 21.337 | 1.00 | 36.38 |
| ATOM | 10806 | CG2 | THR | 5357 | 37.179 | 22.982 | 19.545 | 1.00 | 34.83 |
| ATOM | 10807 | C | THR | 5357 | 38.942 | 24.766 | 20.950 | 1.00 | 36.84 |
| ATOM | 10808 | O | THR | 5357 | 38.228 | 25.633 | 21.439 | 1.00 | 36.48 |
| ATOM | 10809 | N | VAL | 5358 | 39.846 | 25.018 | 20.009 | 1.00 | 37.97 |
| ATOM | 10810 | CA | VAL | 5358 | 40.045 | 26.363 | 19.494 | 1.00 | 39.61 |
| ATOM | 10811 | CB | VAL | 5358 | 41.514 | 26.767 | 19.589 | 1.00 | 39.95 |
| ATOM | 10812 | CG1 | VAL | 5358 | 41.662 | 28.248 | 19.245 | 1.00 | 40.06 |
| ATOM | 10813 | CG2 | VAL | 5358 | 42.038 | 26.461 | 20.992 | 1.00 | 39.25 |
| ATOM | 10814 | C | VAL | 5358 | 39.597 | 26.512 | 18.046 | 1.00 | 40.30 |
| ATOM | 10815 | O | VAL | 5358 | 40.006 | 25.752 | 17.180 | 1.00 | 40.44 |
| ATOM | 10816 | N | LEU | 5359 | 38.754 | 27.502 | 17.787 | 1.00 | 41.63 |
| ATOM | 10817 | CA | LEU | 5359 | 38.269 | 27.751 | 16.434 | 1.00 | 41.67 |
| ATOM | 10818 | CB | LEU | 5359 | 36.741 | 27.681 | 16.406 | 1.00 | 40.73 |
| ATOM | 10819 | CG | LEU | 5359 | 36.108 | 26.480 | 17.115 | 1.00 | 40.18 |
| ATOM | 10820 | CD1 | LEU | 5359 | 34.597 | 26.504 | 16.938 | 1.00 | 39.22 |
| ATOM | 10821 | CD2 | LEU | 5359 | 36.682 | 25.196 | 16.564 | 1.00 | 40.01 |
| ATOM | 10822 | C | LEU | 5359 | 38.741 | 29.126 | 15.966 | 1.00 | 42.37 |
| ATOM | 10823 | O | LEU | 5359 | 38.632 | 29.393 | 14.750 | 1.00 | 43.99 |
| ATOM | 10824 | O | HOH | 6000 | 41.010 | −13.253 | 52.161 | 1.00 | 10.47 |
| ATOM | 10825 | O | HOH | 6001 | 30.281 | 24.620 | 55.046 | 1.00 | 4.40 |
| ATOM | 10826 | O | HOH | 6002 | 18.201 | 21.712 | 42.806 | 1.00 | 15.55 |
| ATOM | 10827 | O | HOH | 6003 | 7.917 | 24.943 | 32.504 | 1.00 | 25.27 |
| ATOM | 10828 | O | HOH | 6004 | 58.961 | 25.219 | 56.650 | 1.00 | 21.60 |
| ATOM | 10829 | O | HOH | 6005 | 5.421 | 18.696 | 22.306 | 1.00 | 25.95 |
| ATOM | 10830 | O | HOH | 6006 | 17.854 | 21.830 | 39.068 | 1.00 | 20.72 |
| ATOM | 10831 | O | HOH | 6007 | 10.847 | 14.908 | 24.899 | 1.00 | 27.69 |
| ATOM | 10832 | O | HOH | 6008 | 18.725 | 15.393 | 11.988 | 1.00 | 25.67 |
| ATOM | 10833 | O | HOH | 6009 | 20.610 | −9.690 | 55.604 | 1.00 | 21.96 |
| ATOM | 10834 | O | HOH | 6010 | 48.787 | 22.614 | 46.276 | 1.00 | 23.92 |
| ATOM | 10835 | O | HOH | 6011 | 7.149 | −8.031 | 2.706 | 1.00 | 38.66 |
| ATOM | 10836 | O | HOH | 6012 | 24.404 | −20.530 | 67.666 | 1.00 | 22.73 |
| ATOM | 10837 | O | HOH | 6013 | 19.332 | −3.585 | 54.974 | 1.00 | 24.69 |
| ATOM | 10838 | O | HOH | 6014 | 47.603 | 19.925 | 56.653 | 1.00 | 25.25 |
| ATOM | 10839 | O | HOH | 6015 | 56.085 | 15.609 | 64.054 | 1.00 | 23.79 |
| ATOM | 10840 | O | HOH | 6016 | 38.389 | 14.678 | 14.371 | 1.00 | 24.18 |
| ATOM | 10841 | O | HOH | 6017 | 26.640 | −13.431 | 69.643 | 1.00 | 30.82 |
| ATOM | 10842 | O | HOH | 6018 | 34.302 | 21.740 | 67.972 | 1.00 | 30.27 |
| ATOM | 10843 | O | HOH | 6019 | 25.230 | 6.654 | 16.151 | 1.00 | 31.32 |
| ATOM | 10844 | O | HOH | 6020 | 29.114 | 18.983 | 48.795 | 1.00 | 18.48 |
| ATOM | 10845 | O | HOH | 6021 | 56.070 | 24.549 | 50.408 | 1.00 | 31.91 |
| ATOM | 10846 | O | HOH | 6022 | 29.477 | 16.898 | 54.810 | 1.00 | 22.80 |
| ATOM | 10847 | O | HOH | 6023 | 22.787 | 11.555 | 7.370 | 1.00 | 30.69 |
| ATOM | 10848 | O | HOH | 6024 | 41.945 | 13.689 | 52.597 | 1.00 | 33.73 |
| ATOM | 10849 | O | HOH | 6025 | 17.054 | 5.525 | 14.250 | 1.00 | 26.83 |
| ATOM | 10850 | O | HOH | 6026 | 63.395 | 28.585 | 68.311 | 1.00 | 20.56 |
| ATOM | 10851 | O | HOH | 6027 | 50.230 | 5.317 | 60.190 | 1.00 | 21.07 |
| ATOM | 10852 | O | HOH | 6028 | 51.038 | 16.487 | 48.964 | 1.00 | 25.10 |
| ATOM | 10853 | O | HOH | 6029 | 10.985 | 11.941 | 29.780 | 1.00 | 33.90 |
| ATOM | 10854 | O | HOH | 6030 | 49.671 | 25.894 | 65.915 | 1.00 | 29.78 |
| ATOM | 10855 | O | HOH | 6031 | 24.930 | 7.181 | 66.296 | 1.00 | 27.29 |
| ATOM | 10856 | O | HOH | 6032 | 27.068 | 13.057 | 71.392 | 1.00 | 26.96 |
| ATOM | 10857 | O | HOH | 6033 | 39.742 | 12.693 | 17.833 | 1.00 | 20.13 |
| ATOM | 10858 | O | HOH | 6034 | 34.266 | 22.639 | 70.388 | 1.00 | 30.04 |
| ATOM | 10859 | O | HOH | 6035 | 25.116 | −12.665 | 36.032 | 1.00 | 26.59 |
| ATOM | 10860 | O | HOH | 6036 | 37.608 | −6.242 | 60.592 | 1.00 | 27.45 |
| ATOM | 10861 | O | HOH | 6037 | 19.811 | 30.238 | 53.158 | 1.00 | 25.23 |
| ATOM | 10862 | O | HOH | 6038 | 50.464 | −3.159 | 69.107 | 1.00 | 18.56 |
| ATOM | 10863 | O | HOH | 6039 | 49.157 | 6.072 | 69.224 | 1.00 | 31.67 |
| ATOM | 10864 | O | HOH | 6040 | 26.110 | 19.612 | 37.487 | 1.00 | 34.79 |
| ATOM | 10865 | O | HOH | 6041 | 23.465 | 18.454 | 30.724 | 1.00 | 28.91 |
| ATOM | 10866 | C1 | SCR | 1 | 18.157 | 11.199 | 8.470 | 1.00 | 65.44 |
| ATOM | 10867 | C2 | SCR | 1 | 17.575 | 12.483 | 7.864 | 1.00 | 65.45 |
| ATOM | 10868 | C3 | SCR | 1 | 18.751 | 13.417 | 7.622 | 1.00 | 64.66 |
| ATOM | 10869 | C4 | SCR | 1 | 19.512 | 13.681 | 8.942 | 1.00 | 64.33 |
| ATOM | 10870 | C5 | SCR | 1 | 20.015 | 12.333 | 9.491 | 1.00 | 63.34 |
| ATOM | 10871 | C6 | SCR | 1 | 20.825 | 12.220 | 10.769 | 1.00 | 61.73 |
| ATOM | 10872 | C11 | SCR | 1 | 17.992 | 8.666 | 6.192 | 1.00 | 69.74 |
| ATOM | 10873 | C12 | SCR | 1 | 18.925 | 9.049 | 7.373 | 1.00 | 68.65 |
| ATOM | 10874 | C13 | SCR | 1 | 20.283 | 8.411 | 7.196 | 1.00 | 69.13 |
| ATOM | 10875 | C14 | SCR | 1 | 20.822 | 8.326 | 8.591 | 1.00 | 69.09 |
| ATOM | 10876 | C15 | SCR | 1 | 19.613 | 7.844 | 9.355 | 1.00 | 68.19 |
| ATOM | 10877 | C16 | SCR | 1 | 19.579 | 8.394 | 10.793 | 1.00 | 66.97 |
| ATOM | 10878 | O1 | SCR | 1 | 18.976 | 10.487 | 7.522 | 1.00 | 66.94 |
| ATOM | 10879 | O2 | SCR | 1 | 16.856 | 12.150 | 6.632 | 1.00 | 66.32 |
| ATOM | 10880 | O22 | SCR | 1 | 15.048 | 12.033 | 5.090 | 1.00 | 67.38 |
| ATOM | 10881 | O23 | SCR | 1 | 14.727 | 13.248 | 7.143 | 1.00 | 66.21 |
| ATOM | 10882 | O24 | SCR | 1 | 14.840 | 10.907 | 7.164 | 1.00 | 66.96 |
| ATOM | 10883 | O3 | SCR | 1 | 18.297 | 14.635 | 7.000 | 1.00 | 64.32 |
| ATOM | 10884 | O32 | SCR | 1 | 17.977 | 14.269 | 4.582 | 1.00 | 63.64 |
| ATOM | 10885 | O33 | SCR | 1 | 18.359 | 16.462 | 5.499 | 1.00 | 63.00 |
| ATOM | 10886 | O34 | SCR | 1 | 20.082 | 14.811 | 5.405 | 1.00 | 64.01 |
| ATOM | 10887 | O4 | SCR | 1 | 20.663 | 14.435 | 8.499 | 1.00 | 65.25 |
| ATOM | 10888 | O42 | SCR | 1 | 22.182 | 16.168 | 8.524 | 1.00 | 65.25 |
| ATOM | 10889 | O43 | SCR | 1 | 21.169 | 15.515 | 10.490 | 1.00 | 65.43 |
| ATOM | 10890 | O44 | SCR | 1 | 19.945 | 16.749 | 8.856 | 1.00 | 65.66 |
| ATOM | 10891 | O5 | SCR | 1 | 18.882 | 11.501 | 9.689 | 1.00 | 64.48 |
| ATOM | 10892 | O6 | SCR | 1 | 20.143 | 12.686 | 11.937 | 1.00 | 60.05 |
| ATOM | 10893 | O62 | SCR | 1 | 21.610 | 13.760 | 13.505 | 1.00 | 59.51 |
| ATOM | 10894 | O63 | SCR | 1 | 21.494 | 11.432 | 13.496 | 1.00 | 58.99 |
| ATOM | 10895 | O64 | SCR | 1 | 19.658 | 12.652 | 14.214 | 1.00 | 59.33 |
| ATOM | 10896 | O10 | SCR | 1 | 18.480 | 8.404 | 8.604 | 1.00 | 68.22 |
| ATOM | 10897 | O51 | SCR | 1 | 18.473 | 7.853 | 11.587 | 1.00 | 65.56 |
| ATOM | 10898 | O52 | SCR | 1 | 16.957 | 9.721 | 11.721 | 1.00 | 64.49 |
| ATOM | 10899 | O53 | SCR | 1 | 18.341 | 9.116 | 13.568 | 1.00 | 65.29 |
| ATOM | 10900 | O54 | SCR | 1 | 16.540 | 7.812 | 12.950 | 1.00 | 64.66 |
| ATOM | 10901 | O71 | SCR | 1 | 22.020 | 7.505 | 8.637 | 1.00 | 69.73 |
| ATOM | 10902 | O72 | SCR | 1 | 24.349 | 7.351 | 9.123 | 1.00 | 70.03 |
| ATOM | 10903 | O73 | SCR | 1 | 22.881 | 8.127 | 10.822 | 1.00 | 70.36 |
| ATOM | 10904 | O74 | SCR | 1 | 23.355 | 9.522 | 9.005 | 1.00 | 70.27 |
| ATOM | 10905 | O81 | SCR | 1 | 17.825 | 7.215 | 6.018 | 1.00 | 71.29 |
| ATOM | 10906 | O82 | SCR | 1 | 15.528 | 7.410 | 5.413 | 1.00 | 72.14 |
| ATOM | 10907 | O83 | SCR | 1 | 16.618 | 5.249 | 5.293 | 1.00 | 72.57 |
| ATOM | 10908 | O84 | SCR | 1 | 17.146 | 6.914 | 3.751 | 1.00 | 71.81 |
| ATOM | 10909 | O91 | SCR | 1 | 21.197 | 9.283 | 6.587 | 1.00 | 70.50 |
| ATOM | 10910 | O92 | SCR | 1 | 22.773 | 9.583 | 4.899 | 1.00 | 71.13 |
| ATOM | 10911 | O93 | SCR | 1 | 20.514 | 9.815 | 4.405 | 1.00 | 70.59 |
| ATOM | 10912 | O94 | SCR | 1 | 21.437 | 7.637 | 4.946 | 1.00 | 70.33 |
| ATOM | 10913 | S2 | SCR | 1 | 15.373 | 12.088 | 6.520 | 1.00 | 67.00 |
| ATOM | 10914 | S3 | SCR | 1 | 18.667 | 15.035 | 5.626 | 1.00 | 63.84 |
| ATOM | 10915 | S4 | SCR | 1 | 20.957 | 15.727 | 9.103 | 1.00 | 65.66 |
| ATOM | 10916 | S6 | SCR | 1 | 20.744 | 12.636 | 13.274 | 1.00 | 59.11 |
| ATOM | 10917 | S11 | SCR | 1 | 16.769 | 6.694 | 5.132 | 1.00 | 72.24 |
| ATOM | 10918 | S12 | SCR | 1 | 21.463 | 9.067 | 5.211 | 1.00 | 71.15 |
| ATOM | 10919 | S13 | SCR | 1 | 23.145 | 8.122 | 9.386 | 1.00 | 70.51 |
| ATOM | 10920 | S14 | SCR | 1 | 17.579 | 8.644 | 12.429 | 1.00 | 64.25 |
| ATOM | 10921 | C1 | SCR | 2 | 34.552 | 0.732 | 83.131 | 1.00 | 83.61 |
| ATOM | 10922 | C2 | SCR | 2 | 34.282 | −0.580 | 83.877 | 1.00 | 83.18 |
| ATOM | 10923 | C3 | SCR | 2 | 35.441 | −1.532 | 83.558 | 1.00 | 82.73 |
| ATOM | 10924 | C4 | SCR | 2 | 35.593 | −1.700 | 82.036 | 1.00 | 82.62 |
| ATOM | 10925 | C5 | SCR | 2 | 35.796 | −0.317 | 81.390 | 1.00 | 82.27 |
| ATOM | 10926 | C6 | SCR | 2 | 35.990 | −0.134 | 79.895 | 1.00 | 81.13 |
| ATOM | 10927 | C11 | SCR | 2 | 35.298 | 3.226 | 85.281 | 1.00 | 88.41 |
| ATOM | 10928 | C12 | SCR | 2 | 35.723 | 2.863 | 83.845 | 1.00 | 87.56 |
| ATOM | 10929 | C13 | SCR | 2 | 37.053 | 3.515 | 83.528 | 1.00 | 88.18 |
| ATOM | 10930 | C14 | SCR | 2 | 37.006 | 3.704 | 82.040 | 1.00 | 87.83 |
| ATOM | 10931 | C15 | SCR | 2 | 35.587 | 4.136 | 81.792 | 1.00 | 87.50 |
| ATOM | 10932 | C16 | SCR | 2 | 35.054 | 3.588 | 80.453 | 1.00 | 87.21 |
| ATOM | 10933 | O1 | SCR | 2 | 35.704 | 1.426 | 83.658 | 1.00 | 85.63 |
| ATOM | 10934 | O2 | SCR | 2 | 34.138 | −0.282 | 85.305 | 1.00 | 83.47 |
| ATOM | 10935 | O22 | SCR | 2 | 32.985 | 0.711 | 87.141 | 1.00 | 83.49 |
| ATOM | 10936 | O23 | SCR | 2 | 32.394 | −1.518 | 86.459 | 1.00 | 83.42 |
| ATOM | 10937 | O24 | SCR | 2 | 31.797 | 0.315 | 85.125 | 1.00 | 83.26 |
| ATOM | 10938 | O3 | SCR | 2 | 35.243 | −2.812 | 84.206 | 1.00 | 82.37 |
| ATOM | 10939 | O32 | SCR | 2 | 35.864 | −2.329 | 86.540 | 1.00 | 81.76 |
| ATOM | 10940 | O33 | SCR | 2 | 35.465 | −4.562 | 85.796 | 1.00 | 81.88 |
| ATOM | 10941 | O34 | SCR | 2 | 37.406 | −3.328 | 85.118 | 1.00 | 81.45 |
| ATOM | 10942 | O4 | SCR | 2 | 36.835 | −2.427 | 81.935 | 1.00 | 83.31 |
| ATOM | 10943 | O42 | SCR | 2 | 38.209 | −4.145 | 81.220 | 1.00 | 83.75 |
| ATOM | 10944 | O43 | SCR | 2 | 36.425 | −3.485 | 79.920 | 1.00 | 84.12 |
| ATOM | 10945 | O44 | SCR | 2 | 36.068 | −4.737 | 81.918 | 1.00 | 84.02 |
| ATOM | 10946 | O5 | SCR | 2 | 34.676 | 0.487 | 81.719 | 1.00 | 82.70 |
| ATOM | 10947 | O6 | SCR | 2 | 34.805 | −0.334 | 79.134 | 1.00 | 79.99 |
| ATOM | 10948 | O62 | SCR | 2 | 35.580 | −1.352 | 77.104 | 1.00 | 79.12 |
| ATOM | 10949 | O63 | SCR | 2 | 35.288 | 0.965 | 77.157 | 1.00 | 79.08 |
| ATOM | 10950 | O64 | SCR | 2 | 33.417 | −0.404 | 77.280 | 1.00 | 79.43 |
| ATOM | 10951 | O10 | SCR | 2 | 34.839 | 3.526 | 82.889 | 1.00 | 87.40 |
| ATOM | 10952 | O51 | SCR | 2 | 33.907 | 4.333 | 79.927 | 1.00 | 87.19 |
| ATOM | 10953 | O52 | SCR | 2 | 32.111 | 3.348 | 81.219 | 1.00 | 87.56 |
| ATOM | 10954 | O53 | SCR | 2 | 32.401 | 2.839 | 78.895 | 1.00 | 86.88 |
| ATOM | 10955 | O54 | SCR | 2 | 31.623 | 4.913 | 79.571 | 1.00 | 87.09 |
| ATOM | 10956 | O71 | SCR | 2 | 38.044 | 4.600 | 81.599 | 1.00 | 87.77 |
| ATOM | 10957 | O72 | SCR | 2 | 39.918 | 4.999 | 80.193 | 1.00 | 87.97 |
| ATOM | 10958 | O73 | SCR | 2 | 38.075 | 3.769 | 79.331 | 1.00 | 88.02 |
| ATOM | 10959 | O74 | SCR | 2 | 39.455 | 2.765 | 80.908 | 1.00 | 88.21 |
| ATOM | 10960 | O81 | SCR | 2 | 34.705 | 4.561 | 85.371 | 1.00 | 90.35 |
| ATOM | 10961 | O82 | SCR | 2 | 33.481 | 4.335 | 87.389 | 1.00 | 91.50 |
| ATOM | 10962 | O83 | SCR | 2 | 34.022 | 6.548 | 86.566 | 1.00 | 91.67 |
| ATOM | 10963 | O84 | SCR | 2 | 35.699 | 5.174 | 87.434 | 1.00 | 91.90 |
| ATOM | 10964 | O91 | SCR | 2 | 38.129 | 2.605 | 83.658 | 1.00 | 89.66 |
| ATOM | 10965 | O92 | SCR | 2 | 40.163 | 1.912 | 84.562 | 1.00 | 90.45 |
| ATOM | 10966 | O93 | SCR | 2 | 38.463 | 2.598 | 85.995 | 1.00 | 90.09 |
| ATOM | 10967 | O94 | SCR | 2 | 39.585 | 4.197 | 84.556 | 1.00 | 90.29 |
| ATOM | 10968 | S2 | SCR | 2 | 32.830 | −0.197 | 85.999 | 1.00 | 83.44 |
| ATOM | 10969 | S3 | SCR | 2 | 35.986 | −3.246 | 85.411 | 1.00 | 81.76 |
| ATOM | 10970 | S4 | SCR | 2 | 36.851 | −3.707 | 81.256 | 1.00 | 83.84 |
| ATOM | 10971 | S6 | SCR | 2 | 34.796 | −0.284 | 77.672 | 1.00 | 79.41 |
| ATOM | 10972 | S11 | SCR | 2 | 34.456 | 5.158 | 86.684 | 1.00 | 91.63 |
| ATOM | 10973 | S12 | SCR | 2 | 39.070 | 2.845 | 84.697 | 1.00 | 90.46 |
| ATOM | 10974 | S13 | SCR | 2 | 38.869 | 4.042 | 80.518 | 1.00 | 87.92 |
| ATOM | 10975 | S14 | SCR | 2 | 32.521 | 3.849 | 79.929 | 1.00 | 87.13 |
| ATOM | 10976 | C1 | SCR | 3 | 30.258 | −0.570 | 4.254 | 1.00 | 100.00 |
| ATOM | 10977 | C2 | SCR | 3 | 30.450 | −1.292 | 2.904 | 1.00 | 99.98 |
| ATOM | 10978 | C3 | SCR | 3 | 29.544 | −2.543 | 2.908 | 1.00 | 100.00 |
| ATOM | 10979 | C4 | SCR | 3 | 29.827 | −3.407 | 4.165 | 1.00 | 100.00 |
| ATOM | 10980 | C5 | SCR | 3 | 29.611 | −2.564 | 5.428 | 1.00 | 99.80 |
| ATOM | 10981 | C6 | SCR | 3 | 29.785 | −3.154 | 6.822 | 1.00 | 99.86 |
| ATOM | 10982 | C11 | SCR | 3 | 28.787 | 2.578 | 4.180 | 1.00 | 100.00 |
| ATOM | 10983 | C12 | SCR | 3 | 28.764 | 1.321 | 5.122 | 1.00 | 100.00 |
| ATOM | 10984 | C13 | SCR | 3 | 27.477 | 1.290 | 5.922 | 1.00 | 100.00 |
| ATOM | 10985 | C14 | SCR | 3 | 27.865 | 0.489 | 7.146 | 1.00 | 100.00 |
| ATOM | 10986 | C15 | SCR | 3 | 29.205 | 1.096 | 7.499 | 1.00 | 100.00 |
| ATOM | 10987 | C16 | SCR | 3 | 30.148 | 0.077 | 8.162 | 1.00 | 99.84 |
| ATOM | 10988 | O1 | SCR | 3 | 28.974 | 0.093 | 4.352 | 1.00 | 100.00 |
| ATOM | 10989 | O2 | SCR | 3 | 30.167 | −0.345 | 1.803 | 1.00 | 100.00 |
| ATOM | 10990 | O22 | SCR | 3 | 30.641 | 1.641 | 0.549 | 1.00 | 99.80 |
| ATOM | 10991 | O23 | SCR | 3 | 31.658 | −0.432 | −0.135 | 1.00 | 100.00 |
| ATOM | 10992 | O24 | SCR | 3 | 32.407 | 0.650 | 1.811 | 1.00 | 100.00 |
| ATOM | 10993 | O3 | SCR | 3 | 29.756 | −3.315 | 1.696 | 1.00 | 100.00 |
| ATOM | 10994 | O32 | SCR | 3 | 27.505 | −4.335 | 1.556 | 1.00 | 100.00 |
| ATOM | 10995 | O33 | SCR | 3 | 28.260 | −2.756 | −0.083 | 1.00 | 100.00 |
| ATOM | 10996 | O34 | SCR | 3 | 29.180 | −4.978 | 0.059 | 1.00 | 100.00 |
| ATOM | 10997 | O4 | SCR | 3 | 28.789 | −4.416 | 4.123 | 1.00 | 100.00 |
| ATOM | 10998 | O42 | SCR | 3 | 27.930 | −6.565 | 4.240 | 1.00 | 100.00 |
| ATOM | 10999 | O43 | SCR | 3 | 29.866 | −6.060 | 5.387 | 1.00 | 99.76 |
| ATOM | 11000 | O44 | SCR | 3 | 29.877 | −6.296 | 3.009 | 1.00 | 100.00 |
| ATOM | 11001 | O5 | SCR | 3 | 30.502 | −1.461 | 5.368 | 1.00 | 99.93 |
| ATOM | 11002 | O6 | SCR | 3 | 31.097 | −2.998 | 7.375 | 1.00 | 99.78 |
| ATOM | 11003 | O62 | SCR | 3 | 31.535 | −5.378 | 7.507 | 1.00 | 100.00 |
| ATOM | 11004 | O63 | SCR | 3 | 33.092 | −3.896 | 8.368 | 1.00 | 100.00 |
| ATOM | 11005 | O64 | SCR | 3 | 32.798 | −3.955 | 6.074 | 1.00 | 100.00 |
| ATOM | 11006 | O10 | SCR | 3 | 29.759 | 1.457 | 6.195 | 1.00 | 100.00 |
| ATOM | 11007 | O51 | SCR | 3 | 29.913 | −0.142 | 9.584 | 1.00 | 100.00 |
| ATOM | 11008 | O52 | SCR | 3 | 31.449 | −2.015 | 9.640 | 1.00 | 99.92 |
| ATOM | 11009 | O53 | SCR | 3 | 29.606 | −1.879 | 11.158 | 1.00 | 100.00 |
| ATOM | 11010 | O54 | SCR | 3 | 31.444 | −0.492 | 11.364 | 1.00 | 100.00 |
| ATOM | 11011 | O71 | SCR | 3 | 26.834 | 0.520 | 8.172 | 1.00 | 100.00 |
| ATOM | 11012 | O72 | SCR | 3 | 25.777 | −0.636 | 9.999 | 1.00 | 99.87 |
| ATOM | 11013 | O73 | SCR | 3 | 27.654 | −1.651 | 8.946 | 1.00 | 100.00 |
| ATOM | 11014 | O74 | SCR | 3 | 25.642 | −1.579 | 7.797 | 1.00 | 100.00 |
| ATOM | 11015 | O81 | SCR | 3 | 29.690 | 3.661 | 4.618 | 1.00 | 100.00 |
| ATOM | 11016 | O82 | SCR | 3 | 31.705 | 3.493 | 5.846 | 1.00 | 100.00 |
| ATOM | 11017 | O83 | SCR | 3 | 31.731 | 4.662 | 3.735 | 1.00 | 100.00 |
| ATOM | 11018 | O84 | SCR | 3 | 31.536 | 2.340 | 3.799 | 1.00 | 99.68 |
| ATOM | 11019 | O91 | SCR | 3 | 26.513 | 0.455 | 5.301 | 1.00 | 100.00 |
| ATOM | 11020 | O92 | SCR | 3 | 24.485 | 0.319 | 4.133 | 1.00 | 100.00 |
| ATOM | 11021 | O93 | SCR | 3 | 26.448 | 0.761 | 2.959 | 1.00 | 100.00 |
| ATOM | 11022 | O94 | SCR | 3 | 25.501 | 2.426 | 4.467 | 1.00 | 100.00 |
| ATOM | 11023 | S2 | SCR | 3 | 31.219 | 0.373 | 1.016 | 1.00 | 100.00 |
| ATOM | 11024 | S3 | SCR | 3 | 28.674 | −3.843 | 0.824 | 1.00 | 100.00 |
| ATOM | 11025 | S4 | SCR | 3 | 29.151 | −5.829 | 4.176 | 1.00 | 100.00 |
| ATOM | 11026 | S6 | SCR | 3 | 32.115 | −4.063 | 7.344 | 1.00 | 99.83 |
| ATOM | 11027 | S11 | SCR | 3 | 31.165 | 3.550 | 4.500 | 1.00 | 100.00 |
| ATOM | 11028 | S12 | SCR | 3 | 25.754 | 1.005 | 4.219 | 1.00 | 100.00 |
| ATOM | 11029 | S13 | SCR | 3 | 26.478 | −0.823 | 8.730 | 1.00 | 100.00 |
| ATOM | 11030 | S14 | SCR | 3 | 30.608 | −1.142 | 10.404 | 1.00 | 100.00 |
| ATOM | 11031 | C1 | SCR | 4 | 46.531 | 12.100 | 82.426 | 1.00 | 99.85 |
| ATOM | 11032 | C2 | SCR | 4 | 46.363 | 13.025 | 83.660 | 1.00 | 99.90 |
| ATOM | 11033 | C3 | SCR | 4 | 46.075 | 14.456 | 83.125 | 1.00 | 99.92 |
| ATOM | 11034 | C4 | SCR | 4 | 47.180 | 14.884 | 82.112 | 1.00 | 99.67 |
| ATOM | 11035 | C5 | SCR | 4 | 47.273 | 13.824 | 81.015 | 1.00 | 99.28 |
| ATOM | 11036 | C6 | SCR | 4 | 48.167 | 13.938 | 79.812 | 1.00 | 98.99 |
| ATOM | 11037 | C11 | SCR | 4 | 43.741 | 9.929 | 81.727 | 1.00 | 100.00 |
| ATOM | 11038 | C12 | SCR | 4 | 44.979 | 10.544 | 81.026 | 1.00 | 100.00 |
| ATOM | 11039 | C13 | SCR | 4 | 44.709 | 10.542 | 79.517 | 1.00 | 100.00 |
| ATOM | 11040 | C14 | SCR | 4 | 46.003 | 10.110 | 78.831 | 1.00 | 100.00 |
| ATOM | 11041 | C15 | SCR | 4 | 46.822 | 9.416 | 79.877 | 1.00 | 100.00 |
| ATOM | 11042 | C16 | SCR | 4 | 48.206 | 10.089 | 79.952 | 1.00 | 100.00 |
| ATOM | 11043 | O1 | SCR | 4 | 45.308 | 11.863 | 81.631 | 1.00 | 100.00 |
| ATOM | 11044 | O2 | SCR | 4 | 45.370 | 12.457 | 84.602 | 1.00 | 100.00 |
| ATOM | 11045 | O22 | SCR | 4 | 44.466 | 11.513 | 86.615 | 1.00 | 99.94 |
| ATOM | 11046 | O23 | SCR | 4 | 46.677 | 12.468 | 86.693 | 1.00 | 100.00 |
| ATOM | 11047 | O24 | SCR | 4 | 46.321 | 10.420 | 85.586 | 1.00 | 100.00 |
| ATOM | 11048 | O3 | SCR | 4 | 45.843 | 15.442 | 84.199 | 1.00 | 100.00 |
| ATOM | 11049 | O32 | SCR | 4 | 48.266 | 15.940 | 84.657 | 1.00 | 100.00 |
| ATOM | 11050 | O33 | SCR | 4 | 46.549 | 17.522 | 85.136 | 1.00 | 99.91 |
| ATOM | 11051 | O34 | SCR | 4 | 46.825 | 15.524 | 86.427 | 1.00 | 100.00 |
| ATOM | 11052 | O4 | SCR | 4 | 46.652 | 16.101 | 81.522 | 1.00 | 100.00 |
| ATOM | 11053 | O42 | SCR | 4 | 47.477 | 17.654 | 79.995 | 1.00 | 99.71 |
| ATOM | 11054 | O43 | SCR | 4 | 48.837 | 16.943 | 81.712 | 1.00 | 99.90 |
| ATOM | 11055 | O44 | SCR | 4 | 47.022 | 18.432 | 82.129 | 1.00 | 99.88 |
| ATOM | 11056 | O5 | SCR | 4 | 47.603 | 12.602 | 81.615 | 1.00 | 99.25 |
| ATOM | 11057 | O6 | SCR | 4 | 47.452 | 13.854 | 78.588 | 1.00 | 98.53 |
| ATOM | 11058 | O62 | SCR | 4 | 49.007 | 15.181 | 77.341 | 1.00 | 98.39 |
| ATOM | 11059 | O63 | SCR | 4 | 46.910 | 16.060 | 77.816 | 1.00 | 98.49 |
| ATOM | 11060 | O64 | SCR | 4 | 47.050 | 14.267 | 76.338 | 1.00 | 98.70 |
| ATOM | 11061 | O10 | SCR | 4 | 46.105 | 9.610 | 81.139 | 1.00 | 100.00 |
| ATOM | 11062 | O51 | SCR | 4 | 49.138 | 9.449 | 80.854 | 1.00 | 100.00 |
| ATOM | 11063 | O52 | SCR | 4 | 48.010 | 9.318 | 83.002 | 1.00 | 99.91 |
| ATOM | 11064 | O53 | SCR | 4 | 49.559 | 11.112 | 82.507 | 1.00 | 100.00 |
| ATOM | 11065 | O54 | SCR | 4 | 50.297 | 8.942 | 82.838 | 1.00 | 100.00 |
| ATOM | 11066 | O71 | SCR | 4 | 45.740 | 9.331 | 77.627 | 1.00 | 100.00 |
| ATOM | 11067 | O72 | SCR | 4 | 46.494 | 8.435 | 75.533 | 1.00 | 100.00 |
| ATOM | 11068 | O73 | SCR | 4 | 48.123 | 9.130 | 77.089 | 1.00 | 99.61 |
| ATOM | 11069 | O74 | SCR | 4 | 46.862 | 10.762 | 75.987 | 1.00 | 100.00 |
| ATOM | 11070 | O81 | SCR | 4 | 44.003 | 9.561 | 83.129 | 1.00 | 100.00 |
| ATOM | 11071 | O82 | SCR | 4 | 43.951 | 8.169 | 85.042 | 1.00 | 100.00 |
| ATOM | 11072 | O83 | SCR | 4 | 44.216 | 7.142 | 82.863 | 1.00 | 100.00 |
| ATOM | 11073 | O84 | SCR | 4 | 42.165 | 8.092 | 83.495 | 1.00 | 100.00 |
| ATOM | 11074 | O91 | SCR | 4 | 44.576 | 11.859 | 79.014 | 1.00 | 100.00 |
| ATOM | 11075 | O92 | SCR | 4 | 43.455 | 13.408 | 77.692 | 1.00 | 100.00 |
| ATOM | 11076 | O93 | SCR | 4 | 42.545 | 12.815 | 79.750 | 1.00 | 100.00 |
| ATOM | 11077 | O94 | SCR | 4 | 42.584 | 11.235 | 77.915 | 1.00 | 100.00 |
| ATOM | 11078 | S2 | SCR | 4 | 45.716 | 11.716 | 85.866 | 1.00 | 100.00 |
| ATOM | 11079 | S3 | SCR | 4 | 46.874 | 16.094 | 85.086 | 1.00 | 100.00 |
| ATOM | 11080 | S4 | SCR | 4 | 47.499 | 17.289 | 81.373 | 1.00 | 100.00 |
| ATOM | 11081 | S6 | SCR | 4 | 47.616 | 14.849 | 77.536 | 1.00 | 98.29 |
| ATOM | 11082 | S11 | SCR | 4 | 43.605 | 8.235 | 83.631 | 1.00 | 100.00 |
| ATOM | 11083 | S12 | SCR | 4 | 43.282 | 12.307 | 78.605 | 1.00 | 100.00 |
| ATOM | 11084 | S13 | SCR | 4 | 46.799 | 9.413 | 76.568 | 1.00 | 100.00 |
| ATOM | 11085 | S14 | SCR | 4 | 49.214 | 9.707 | 82.295 | 1.00 | 100.00 |
| ATOM | 11086 | S | SO4 | 9001 | −0.201 | 33.666 | 31.694 | 1.00 | 70.52 |
| ATOM | 11087 | O1 | SO4 | 9001 | −1.549 | 33.115 | 31.925 | 1.00 | 70.57 |
| ATOM | 11088 | O2 | SO4 | 9001 | −0.211 | 35.121 | 31.935 | 1.00 | 70.60 |
| ATOM | 11089 | O3 | SO4 | 9001 | 0.736 | 33.022 | 32.636 | 1.00 | 70.65 |
| ATOM | 11090 | O4 | SO4 | 9001 | 0.201 | 33.418 | 30.292 | 1.00 | 70.21 |
| ATOM | 11091 | S | SO4 | 9002 | 67.211 | 34.040 | 57.975 | 1.00 | 75.75 |
| ATOM | 11092 | O1 | SO4 | 9002 | 67.310 | 35.125 | 56.981 | 1.00 | 75.62 |
| ATOM | 11093 | O2 | SO4 | 9002 | 68.562 | 33.697 | 58.463 | 1.00 | 75.61 |
| ATOM | 11094 | O3 | SO4 | 9002 | 66.372 | 34.466 | 59.119 | 1.00 | 75.40 |
| ATOM | 11095 | O4 | SO4 | 9002 | 66.614 | 32.866 | 57.313 | 1.00 | 75.52 |
| ATOM | 11096 | S | SO4 | 9003 | 25.939 | −22.811 | 81.082 | 1.00 | 78.81 |
| ATOM | 11097 | O1 | SO4 | 9003 | 26.791 | −23.418 | 80.039 | 1.00 | 79.05 |
| ATOM | 11098 | O2 | SO4 | 9003 | 26.025 | −23.616 | 82.319 | 1.00 | 79.12 |
| ATOM | 11099 | O3 | SO4 | 9003 | 26.394 | −21.432 | 81.355 | 1.00 | 79.12 |
| ATOM | 11100 | O4 | SO4 | 9003 | 24.541 | −22.775 | 80.610 | 1.00 | 79.18 |
| END | | | | | | | | | |

1. An isolated composition comprising a ternary complex of:
(a) an FGF ligand polypeptide;
(b) an FGF receptor polypeptide; and
(c) a heparin agonist or antagonist,
wherein the heparin agonist or antagonist binds to the FGF ligand polypeptide and the FGF receptor polypeptide to form the ternary complex.

2. An isolated composition according to claim 1 in which the FGF ligand polypeptide is an FGF2 polypeptide having the amino acid sequence set forth in SEQ ID NO:1.

3. An isolated composition according to claim 1 in which the FGF receptor polypeptide is an FGFR1 polypeptide comprising residues 142-365 of the amino acid sequence set forth in SEQ ID NO:3.

4. An isolated composition according to claim 1 in which the heparin agonist or antagonist is a compound having the structure: 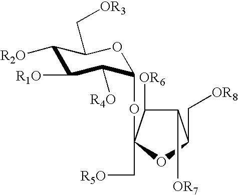
wherein R₁, R₂, R₃, R₄, R₅, R₆, R₇ and R₈ are independently benzyl, trityl, or —SO₃H.

5. An isolated composition according to claim 4 wherein at least one of R₁, R₂, R₃, R₄, R₅, R₆, R₇ and R₈ is a benzyl or trityl.

6. An isolated composition according to claim 4 in which the heparin agonist or antagonist is a heparin agonist.

7. An isolated composition according to claim 6 in which the heparin agonist is sucrose octasulfate (SOS).

8. An isolated composition according to claim 6 in which the heparin agonist is inositol hexasulfate or cyclodextrin.

9. An isolated composition according to claim 4 in which the heparin agonist or antagonist is a heparin antagonist.

10. An isolated composition according to claim 4 in which the heparin antagonist is a compound having the structure: 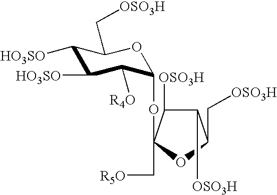
wherein R₄ and R₅ are independently benzyl, trityl, or SO₃H, and
wherein at least one of R₄ and R₅ is benzyl or trityl.

11. An isolated composition according to claim 1 in which the ternary complex is dimerized.

12. An isolated composition according to claim 1 in which the ternary complex is dimer incompetent.

13. An isolated composition according to claim 1 in which molecules of the ternary complex have a crystalline structure.

14. An isolated composition according to claim 13 in which the crystalline structure has structure coordinates as set forth in the Appendix.

15. A method for identifying a compound that is an inhibitor of FGF receptor activity, which method comprises:
(a) designing a test compound, based on crystal structure coordinates for a ternary complex comprising (i) an FGF ligand polypeptide, (ii) an FGF receptor polypeptide, and (iii) a heparin agonist or antagonist that binds to the FGF ligand polypeptide and the FGF receptor polypeptide to form the ternary complex;
(b) synthesizing the designed test compound; and
(c) determining whether the test compound modulates FGF receptor activity.

16. A method according to claim 15 in which:
(a) a first ternary complex and a second ternary complex are dimerized in the crystal structure coordinates; and
(b) the test compound is designed to form hydrogen bonds with the FGF receptor and ligand polypeptides in the first ternary complex, and also to form hydrogen bonds with an FGF receptor in the second ternary complex.

17. A method according to claim 15 in which the FGF receptor activity is a tyrosine kinase activity.

18. A method according to claim 15 in which the FGF receptor activity is an activity selected from the group consisting of mitogenesis and angiogenesis.

19. A method for inhibiting FGF receptor activity in a cell expressing an FGF receptor polypeptide, which method comprises contacting the cell with a compound in the presence of an FGF ligand so that FGF receptor activity in the cell is inhibited,
the compound having the structure: 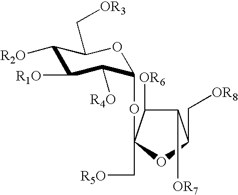
wherein R₁, R₂, R₃, R₄, R₅, R₆, R₇ and R₈ are independently benzyl, trityl, or —SO₃H, and at least one of R₁, R₂, R₃, R₄, R₅, R₆, R₇ and R₈ is benzyl or trityl.

20. A method according to claim 19, wherein the compound has the structure 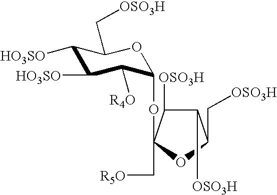
wherein R₄ and R₅ are independently benzyl, trityl or —SO3H, and
wherein at least one of R₄ and R₅ is benzyl or trityl.

21. A method according to claim 19 in which the FGF receptor activity is a tyrosine kinase activity.

22. A method according to claim 19 in which the FGF receptor activity is angiogenesis or mitogenesis.

23. A method for inhibiting dimerization of an FGF receptor polypeptide, which method comprises contacting the FGF receptor polypeptide to an admixture comprising (i) an FGF ligand, and (ii) having the structure: 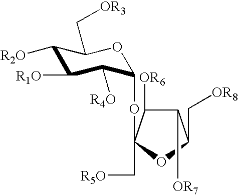
wherein R₁, R₂, R₃, R₄, R₅, R₆, R₇ and R₈ are independently benzyl, trityl, or —SO₃H, and at least one of R₁, R₂, R₃, R₄, R₅, R₆, R₇ and R₈ is benzyl or trityl,
so that dimerization of the FGF receptor polypeptide is inhibited.

24. A method according to claim 19, wherein the compound has the structure 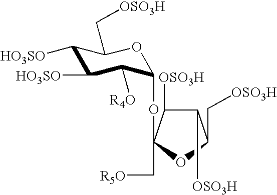
wherein R₄ and R₅ are independently benzyl, trityl or —SO3H, and
wherein at least one of R₄ and R₅ is benzyl or trityl.

25. A pharmaceutical composition comprising:
(a) as compound having the structure: 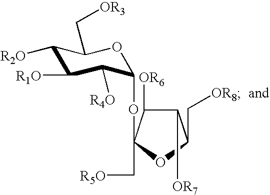
(b) a physiologically acceptable carrier or excipient,
wherein R₁, R₂, R₃, R₄, R₅, R₆, R₇ and R₈ are independently benzyl, trityl, or —SO₃H, and at least one of R₁, R₂, R₃, R₄, R₅, R₆, R₇ and R₈ is benzyl or trityl.

26. A pharmaceutical composition according to claim 25, wherein the compound has the structure: 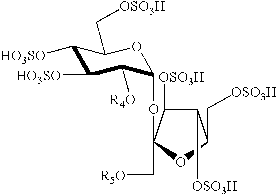
wherein R₄ and R₅ are independently benzyl, trityl or —SO3H, and
wherein at least one of R₄ and R₅ is benzyl or trityl.

27. An isolated composition according to claim 9, wherein the heparin antagonist is suramin.
